(19) United States
(12) Patent Application Publication
(10) Pub. No.: US 2025/0326823 A1
(43) Pub. Date: Oct. 23, 2025

(54) Antigen Binding Polypeptides, Polypeptide Complexes and Methods of Use Thereof

(71) Applicants: ModeX Therapeutics, Inc., Weston, MA (US); The United States of America, as Represented by the Secretary, Dept. of Health and Human Services, Bethesda, MD (US)

(72) Inventors: Juan LI, Waltham, MA (US); Peng HE, Sharon, MA (US); Chih-Jen WEI, Weston, MA (US); Gary J. NABEL, Delray Beach, FL (US); John R. MASCOLA, Rockville, MD (US); Heather Dawn KAMP, Westwood, MA (US); Manjie HUANG, Medford, MA (US); John MISASI, Kensington, MD (US); Amarendra PEGU, Potomac, MD (US); Lingshu WANG, Bethesda, MD (US); Tongqing ZHOU, Bethesda, MD (US); Nicole DORIA-ROSE, Bethesda, MD (US); Eun Sung YANG, Bethesda, MD (US); Wei SHI, Bethesda, MD (US); Qi QIU, Bethesda, MD (US); Qiong ZHOU, Bethesda, MD (US); Peter D. KWONG, Washington, DC (US); Richard A. KOUP, Bethesda, MD (US); Theodore PIERSON, Bethesda, MD (US); Nancy J. SULLIVAN, Boston, MA (US)

(21) Appl. No.: 19/170,000

(22) Filed: Apr. 3, 2025

Related U.S. Application Data
(60) Provisional application No. 63/574,175, filed on Apr. 3, 2024.

(51) Int. Cl. C07K 16/10; C07K 16/30

(52) U.S. Cl. CPC C07K 16/1003; C07K 16/30; C07K 2317/31

(57) ABSTRACT

Disclosed are antigen binding polypeptides and antigen binding polypeptide complexes (e.g., antibodies and antigen binding fragments thereof) having certain structural features. Also disclosed are polynucleotides and vectors encoding such polypeptides and polypeptide complexes; host cells, pharmaceutical compositions and kits containing such polypeptides and polypeptide complexes; and methods of using such polypeptides and polypeptide complexes.

### CROSS-REFERENCE TO RELATED APPLICATION

This application claims priority benefit to U.S. Provisional Appl. No. 63/574,175, filed Apr. 3, 2024, the contents of which are hereby incorporated by reference in its entirety.

### STATEMENT REGARDING FEDERALLY SPONSORED RESEARCH OR DEVELOPMENT

This invention was made in the performance of a Cooperative Research and Development Agreement with the National Institutes of Health, an Agency of the Department of Health and Human Services. The Government has certain rights in this invention.

### REFERENCE TO SEQUENCE LISTING SUBMITTED ELECTRONICALLY

The content of the electronically submitted Sequence Listing XML (Name: 4850_0150001_SequenceListing_ST26.xml; Size: 2,631,424 bytes; and Date of Creation: Apr. 2, 2025), filed with the application, is incorporated herein by reference in its entirety.

### FIELD OF THE INVENTION

The present disclosure relates to anti-severe acute respiratory syndrome coronavirus 2 (anti-SARS-CoV-2) antigen binding polypeptides and polypeptide complexes (e.g., antibodies and antigen binding fragments thereof) having certain structural features. The present disclosure further relates to antigen binding polypeptides and polypeptide complexes (e.g., antibodies and antigen binding fragments thereof), having certain structural features, which have the ability to bind to one or more antigens, such as infectious disease antigens (including viral antigens (including viral antigens that are not sarbecovirus antigens), bacterial antigens, and parasite antigens); tumor-associated antigens; or antigens associated with pathologies other than infectious diseases and cancer/tumor. The present disclosure also relates to polynucleotides and vectors encoding such polypeptides and polypeptide complexes; host cells; pharmaceutical compositions and kits containing such polypeptides and polypeptide complexes; and methods of using such polypeptides and polypeptide complexes.

### BACKGROUND

The outbreak of severe acute respiratory syndrome coronavirus (SARS-CoV-2) has caused over 450 million infections and over 6.9 million deaths worldwide. SARS-CoV-2 is the strain of coronavirus that causes COVID-19 (coronavirus disease 2019). Sequencing and publication of the original Wuhan Hu-1 (WA-1) genome enabled rapid vaccine design and production of spike proteins for therapeutic antibody development. However, persistent circulation of viruses during human pandemics leads to genetic variation that resists containment and eradication. While cellular and humoral immune responses are needed for optimal protection against viral infection, neutralizing antibodies play a major role in prevention and eradication of pathogens.

SARS-CoV-2 spike protein mutants and SARS-CoV-2 variants D614G spike protein mutant (e.g., D614G). Alpha variant (e.g., B.1.1.7). Beta variant (e.g., B.1.351). Gamma variant (e.g., P.1). Delta variant (e.g., B.1.617.2 or AY.1). Kappa variant (e.g., B.1.617.1). Iota variant (e.g., B.1.526). Lambda variant (e.g., C.37). Epsilon variant (e.g., B.1.427), Mu variant (e.g., B.1.621). Theta variant. Zeta variant. R.1 variant. C.1.2 variant, and Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, or KP.3.1.1) have emerged to contain mutations that are resistant to current therapies, have increased pathogenicity, and evade current vaccines. This led to their classification as variants of concern (VOCs).

Therapeutic antibodies have emerged as an important class of agents for treating human diseases and disorders. Therapeutic antibodies have been engineered to have specificity for one or two or more different antigens or epitopes. “Monospecific” antibodies, including engineered monospecific antibodies, are antibodies that have specificity for one antigen or epitope. “Multispecific” antibodies are, for example, bispecific, trispecific, or tetraspecific antibodies, which are antibodies that have been engineered to have specificity for two, three, or four different antigens or epitopes, respectively. Monospecific and multispecific antibodies have been used to form multi-targeting strategies to treat human diseases and disorders, and are an important technological platform for developing neutralizing antibody-based therapeutics for preventing and treating SARS-CoV-2 infection and COVID-19.

There is a need for improved therapies to prevent and treat SARS-CoV-2 infection and COVID-19. There is a need for therapeutic antibodies with broad VOC reactivity and resistance to SARS-CoV-2 escape. There is also a need for therapeutic antibodies that are easier to manufacture and administer than current therapeutic antibody cocktails. There is also a need to treat or prevent SARS-CoV-2 infection and COVID-19 in immunocompromised individuals who cannot mount an effective immune response through vaccinations.

Therapeutic antibodies have emerged as an important class of agents for treating human diseases and disorders. Therapeutic antibodies have been engineered to have specificity for one or two or more different antigens or epitopes. “Monospecific” antibodies, including engineered monospecific antibodies, are antibodies that have specificity for one antigen or epitope. “Multispecific” antibodies are, for example, bispecific, trispecific, or tetraspecific antibodies, which are antibodies that have been engineered to have specificity for two, three, or four different antigens or epitopes, respectively. Monospecific and multispecific antibodies have been used to form multi-targeting strategies to treat human pathologies, and are an important technological platform for developing antibody-based therapeutics for preventing and treating pathologies, such as infectious diseases (such as viral infections (including non-sarbecovirus infections), bacterial infections, and parasite infections): cancers/tumors; and pathologies other than infectious diseases and cancers/tumors.

There is a need for improved therapies to prevent and treat pathologies. There is also a need for therapeutic antibodies that are easier to manufacture and administer than current therapeutic antibody cocktails. There is also a need to treat or prevent pathologies in immunocompromised individuals who cannot mount an effective immune response through vaccinations.

### BRIEF SUMMARY

Provided herein is an antigen binding polypeptide having a structure, from amino-terminus to carboxy-terminus, represented by: VL1-L1-CL-L2-VL2-L3-VH2-L4-VH1-L5-CH1; VL1-L1-CL-L2-VH2-L3-VL2-L4-VH1-L5-CH1; VL1-L1-CH1-L2-VL2-L3-VH2-L4-VH1-L5-CL; VL1-L1-CH1-L2- VH2-L3-VL2-L4-VH1-L5-CL; VH1-L1-CL-L2-VL2-L3-VH2-L4-VL1-L5-CH1; VH1-L1-CL-L2-VH2-L3-VL2-L4-VL1-L5-CH1; VH1-L1-CH1-L2-VL2-L3-VH2-L4-VL1-L5-CL; VH1-L1-CH1-L2-VH2-L3- VL2-L4-VL1-L5-CL; VL2-L1-CL-L2-VL1-L3-VH1-L4-VH2-L5-CH1; VL2-L1-CL-L2-VH1-L3-VL1-L4-VH2-L5-CH1; VL2-L1-CH1-L2-VL1-L3-VH1-L4-VH2-L5-CL; VL2-L1-CH1-L2-VH1-L3-VL1-L4-VH2-L5-CL; VH2-L1-CL-L2-VL1-L3-VH1-L4-VL2-L5-CH1; VH2-L1-CL-L2-VH1-L3-VL1-L4-VL2-L5-CH1; VH2-L1-CH1-L2-VL1-L3-VH1-L4-VL2-L5-CL; or VH2-L1-CH1-L2-VH1-L3-VL1-L4-VL2-L5-CL; wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CL is an immunoglobulin light chain constant region; CH1 is an immunoglobulin heavy chain constant region 1; and L1-L5 are optional amino acid linkers.

Also provided herein is an antigen binding polypeptide having a structure, from amino-terminus to carboxy-terminus, represented by: VL1-CL-L1-VL2-L2-VH2-L3-VH1-CH1; VL1-CL-L1-VH2-L2-VL2-L3-VH1-CH1; VL1-CH1-L1-VL2-L2-VH2-L3-VH1-CL; VL1-CH1-L1-VH2-L2-VL2-L3-VH1-CL; VH1-CL-L1-VL2-L2-VH2-L3-VL1-CH1; VH1-CL-L1-VH2-L2-VL2-L3-VL1-CH1; VH1-CH1-L1-VL2-L2-VH2-L3-VL1-CL; VH1-CH1-L1-VH2-L2-VL2-L3-VL1-CL; VL2-CL-L1-VL1-L2-VH1-L3-VH2-CH1; VL2-CL-L1-VH1-L2-VL1-L3-VH2-CH1; VL2-CH1-L1-VL1-L2-VH1-L3-VH2-CL; VL2-CH1-L1-VH1-L2-VL1-L3-VH2-CL; VH2-CL-L1-VL1-L2-VH1-L3-VL2-CH1; VH2-CL-L1-VH1-L2-VL1-L3-VL2-CH1; VH2-CH1-L1-VL1-L2-VH1-L3-VL2-CL; or VH2-CH1-L1-VH1-L2-VL1-L3-VL2-CL; wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CL is an immunoglobulin light chain constant region; CH1 is an immunoglobulin heavy chain constant region 1; and L1-L3 are amino acid linkers

Also provided herein is an antigen binding polypeptide having a structure, from amino-terminus to carboxy-terminus, represented by: VL1-L1-VH1-L2-VL2-L3-CL-L4-VH2-L5-CH1; VL1-L1-VH1-L2-VL2-L3-CH1-L4-VH2-L5-CL; VL1-L1-VH1-L2-VH2-L3-CL-L4-VL2-L5-CH1; VL1-L1-VH1-L2-VH2-L3-CH1-L4-VL2-L5-CL; VL1-L1-VL2-L2-VH1-L3-CL-L4-VH2-L5-CH1; VL1-L1-VL2-L2-VH1-L3-CH1-L4-VH2-L5-CL; VL1-L1-VH2-L2-VH1-L3-CL-L4-VL2-L5-CH1; VL1-L1-VH2-L2-VH1-L3-CH1-L4-VL2-L5-CL; VH1-L1-VL1-L2-VL2-L3-CL-L4-VH2-L5-CH1; VH1-L1-VL1-L2-VL2-L3-CH1-L4-VH2-L5-CL; VH1-L1-VL1-L2-VH2-L3-CL-L4-VL2-L5-CH1; VH1-L1-VL1-L2-VH2-L3-CH1-L4-VL2-L5-CL; VH1-L1-VL2-L2-VL1-L3-CL-L4-VH2-L5-CH1; VH1-L1-VL2-L2-VL1-L3-CH1-L4-VH2-L5-CL; VH1-L1-VH2-L2-VL1-L3-CL-L4-VL2-L5-CH1; VH1-L1-VH2-L2-VL1-L3-CH1-L4-VL2-L5- CL; VL2-L1-VL1-L2-VH2-L3-CL-L4-VH1-L5-CH1; VL2-L1-VL1-L2-VH2-L3-CH1-L4-VH1-L5-CL; VL2-L1-VH1-L2-VH2-L3-CL-L4-VL1-L5-CH1; VL2-L1-VH1-L2-VH2-L3-CH1-L4-VL1-L5-CL; VL2-L1-VH2-L2-VL1-L3-CL-L4-VH1-L5-CH1; VL2-L1-VH2-L2-VL1-L3-CH1-L4-VH1-L5-CL; VL2-L1-VH2-L2-VH1-L3-CL-L4-VL1-L5-CH1; VL2-L1-VH2-L2-VH1-L3-CH1-L4-VL1-L5-CL; VH2-L1-VL1-L2-VL2-L3-CL-L4-VH1-L5-CH1; VH2-L1-VL1-L2-VL2-L3-CH1-L4-VH1-L5-CL; VH2-L1-VH1-L2-VL2-L3-CL-L4-VL1-L5-CH1; VH2-L1-VH1-L2-VL2-L3-CH1-L4-VL1-L5-CL; VH2-L1-VL2-L2-VL1-L3-CL-L4-VH1-L5-CH1; VH2-L1-VL2-L2-VL1-L3-CH1-L4-VH1-L5-CL; VH2-L1-VL2-L2-VH1-L3-CL-L4-VL1-L5-CH1; or VH2-L1-VL2-L2-VH1-L3-CH1-L4-VL1-L5-CL; wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CL is an immunoglobulin light chain constant region; CH1 is an immunoglobulin heavy chain constant region 1; and L1-L5 are optional amino acid linkers.

Also provided herein is an antigen binding polypeptide having a structure, from amino-terminus to carboxy-terminus, represented by: VL1-L1-CL-L2-VH1-L3-CH1-L4-VL2-L5-VH2; VL1-L1-CL-L2-VH1-L3-CH1-L4-VH2-L5-VL2; VL1-L1-CL-L2-VL2-L3-CH1-L4-VH1-L5-VH2; VL1-L1-CL-L2-VH2-L3-CH1-L4-VH1-L5-VL2; VL1-L1-CH1-L2-VH1-L3-CL-L4-VL2-L5-VH2; VL1-L1-CH1-L2-VH1-L3-CL-L4-VH2-L5-VL2; VL1-L1-CH1-L2-VL2-L3-CL-L4-VH1-L5-VH2; VL1-L1-CH1-L2-VH2-L3-CL- L4-VH1-L5-VL2; VH1-L1-CL-L2-VL1-L3-CH1-L4-VL2-L5-VH2; VH1-L1-CL-L2-VL1-L3-CH1-L4-VH2-L5-VL2; VH1-L1-CL-L2-VL2-L3-CH1-L4-VL1-L5-VH2; VH1-L1-CL-L2-VH2-L3-CH1-L4-VL1-L5-VL2; VH1-L1-CH1-L2-VL1-L3-CL-L4-VL2-L5-VH2; VH1-L1-CH1-L2-VL1-L3-CL-L4-VH2-L5-VL2; VH1-L1-CH1-L2-VL2-L3-CL-L4-VL1-L5-VH2; VH1-L1-CH1-L2-VH2-L3-CL-L4-VL1-L5-VL2; VL2-L1-CL-L2-VL1-L3-CH1-L4-VH2-L5-VH1; VL2-L1-CL-L2-VH1-L3-CH1-L4-VH2-L5-VL1; VL2-L1-CL-L2-VH2-L3-CH1-L4-VL1-L5-VH1; VL2-L1-CL-L2-VH2-L3-CH1-L4-VH1-L5-VL1; VL2-L1-CH1-L2-VL1-L3-CL-L4-VH2-L5-VH1; VL2-L1-CH1-L2-VH1-L3-CL-L4-VH2-L5-VL1; VL2-L1-CH1-L2-VH2-L3-CL-L4-VL1-L5-VH1; VL2-L1-CH1-L2-VH2-L3-CL-L4-VH1-L5-VL1; VH2-L1-CL-L2-VL1-L3-CH1-L4-VL2-L5-VH1; VH2-L1-CL-L2-VH1-L3-CH1-L4-VL2-L5-VL1; VH2-L1-CL-L2-VL2-L3-CH1-L4-VL1-L5-VH1; VH2-L1-CL-L2-VL2-L3-CH1-L4-VH1-L5-VL1; VH2-L1-CH1-L2-VL1-L3-CL-L4-VL2-L5-VH1; VH2-L1-CH1-L2-VH1-L3-CL-L4-VL2-L5-VL1; VH2-L1-CH1-L2-VL2-L3-CL-L4-VL1-L5-VH1; or VH2-L1-CH1-L2-VL2-L3-CL-L4-VH1-L5-VL1; wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CL is an immunoglobulin light chain constant region; CH1 is an immunoglobulin heavy chain constant region 1; and L1-L5 are optional amino acid linkers.

In some aspects and in any one of the formulas depicted herein (a) VL1 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; (b) VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069; (c) VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; and (d) VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects and in any one of the formulas depicted herein (a) VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; (b) VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884; (c) VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; and (d) VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884.

In some aspects and in any one of the formulas shown herein, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 17; (b) VL2 comprises an amino acid sequence that is at least 65%, at least 70%, at least 75%, at least 80%, at least 85%, at least 90%, at least 95%, at least 99%, or 100% identical to SEQ ID NO: 25; (c) VH1 comprises an amino acid sequence that is at least 65%, at least 70%, at least 75%, at least 80%, at least 85%, at least 90%, at least 95%, at least 99%, or 100% identical to SEQ ID NO: 21 or 22; and (d) VH2 comprises an amino acid sequence that is at least 65%, at least 70%, at least 75%, at least 80%, at least 85%, at least 90%, at least 95%, at least 99%, or 100% identical to SEQ ID NO: 29.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 17; (b) VL2 comprises an amino acid sequence that is at least 65%, at least 70%, at least 75%, at least 80%, at least 85%, at least 90%, at least 95%, at least 99%, or 100% identical to SEQ ID NO: 11; (c) VH1 comprises an amino acid sequence that is at least 65%, at least 70%, at least 75%, at least 80%, at least 85%, at least 90%, at least 95%, at least 99%, or 100% identical to SEQ ID NO: 21 or 22; and (d) VH2 comprises an amino acid sequence that is at least 65%, at least 70%, at least 75%, at least 80%, at least 85%, at least 90%, at least 95%, at least 99%, or 100% identical to SEQ ID NO: 14.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 25; (b) VL2 comprises an amino acid sequence that is at least 65%, at least 70%, at least 75%, at least 80%, at least 85%, at least 90%, at least 95%, at least 99%, or 100% identical to SEQ ID NO: 11; (c) VH1 comprises an amino acid sequence that is at least 65%, at least 70%, at least 75%, at least 80%, at least 85%, at least 90%, at least 95%, at least 99%, or 100% identical to SEQ ID NO: 29; and (d) VH2 comprises an amino acid sequence that is at least 65%, at least 70%, at least 75%, at least 80%, at least 85%, at least 90%, at least 95%, at least 99%, or 100% identical to SEQ ID NO: 14.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 25; (b) VL2 comprises an amino acid sequence that is at least 65%, at least 70%, at least 75%, at least 80%, at least 85%, at least 90%, at least 95%, at least 99%, or 100% identical to SEQ ID NO: 4; (c) VH1 comprises an amino acid sequence that is at least 65%, at least 70%, at least 75%, at least 80%, at least 85%, at least 90%, at least 95%, at least 99%, or 100% identical to SEQ ID NO: 29; and (d) VH2 comprises an amino acid sequence that is at least 65%, at least 70%, at least 75%, at least 80%, at least 85%, at least 90%, at least 95%, at least 99%, or 100% identical to SEQ ID NO: 8.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 17; (b) VL2 comprises an amino acid sequence that is at least 65%, at least 70%, at least 75%, at least 80%, at least 85%, at least 90%, at least 95%, at least 99%, or 100% identical to SEQ ID NO: 33 or 39; (c) VH1 comprises an amino acid sequence that is at least 65%, at least 70%, at least 75%, at least 80%, at least 85%, at least 90%, at least 95%, at least 99%, or 100% identical to SEQ ID NO: 21 or 22; and (d) VH2 comprises an amino acid sequence that is at least 65%, at least 70%, at least 75%, at least 80%, at least 85%, at least 90%, at least 95%, at least 99%, or 100% identical to SEQ ID NO: 37.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 11; (b) VL2 comprises an amino acid sequence that is at least 65%, at least 70%, at least 75%, at least 80%, at least 85%, at least 90%, at least 95%, at least 99%, or 100% identical to SEQ ID NO: 67; (c) VH1 comprises an amino acid sequence that is at least 65%, at least 70%, at least 75%, at least 80%, at least 85%, at least 90%, at least 95%, at least 99%, or 100% identical to SEQ ID NO: 14; and (d) VH2 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 147; (b) VL2 comprises an amino acid sequence that is at least 65%, at least 70%, at least 75%, at least 80%, at least 85%, at least 90%, at least 95%, at least 99%, or 100% identical to SEQ ID NO: 11; (c) VH1 comprises an amino acid sequence that is at least 65%, at least 70%, at least 75%, at least 80%, at least 85%, at least 90%, at least 95%, at least 99%, or 100% identical to SEQ ID NO: 151; and (d) VH2 comprises an amino acid sequence that is at least 65%, at least 70%, at least 75%, at least 80%, at least 85%, at least 90%, at least 95%, at least 99%, or 100% identical to SEQ ID NO: 14.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 67; (b) VL2 comprises an amino acid sequence that is at least 65%, at least 70%, at least 75%, at least 80%, at least 85%, at least 90%, at least 95%, at least 99%, or 100% identical to SEQ ID NO: 17; (c) VH1 comprises an amino acid sequence that is at least 65%, at least 70%, at least 75%, at least 80%, at least 85%, at least 90%, at least 95%, at least 99%, or 100% identical to SEQ ID NO: 71 or 626; and (d) VH2 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 67; (b) VL2 comprises an amino acid sequence that is at least 65%, at least 70%, at least 75%, at least 80%, at least 85%, at least 90%, at least 95%, at least 99%, or 100% identical to SEQ ID NO: 154; (c) VH1 comprises an amino acid sequence that is at least 65%, at least 70%, at least 75%, at least 80%, at least 85%, at least 90%, at least 95%, at least 99%, or 100% identical to SEQ ID NO: 71 or 626; and (d) VH2 comprises an amino acid sequence that is at least 65%, at least 70%, at least 75%, at least 80%, at least 85%, at least 90%, at least 95%, at least 99%, or 100% identical to SEQ ID NO: 158.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 17; (b) VL2 comprises an amino acid sequence that is at least 65%, at least 70%, at least 75%, at least 80%, at least 85%, at least 90%, at least 95%, at least 99%, or 100% identical to SEQ ID NO: 67; (c) VH1 comprises an amino acid sequence that is at least 65%, at least 70%, at least 75%, at least 80%, at least 85%, at least 90%, at least 95%, at least 99%, or 100% identical to SEQ ID NO: 21 or 22; and (d) VH2 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 154; (b) VL2 comprises an amino acid sequence that is at least 65%, at least 70%, at least 75%, at least 80%, at least 85%, at least 90%, at least 95%, at least 99%, or 100% identical to SEQ ID NO: 67; (c) VH1 comprises an amino acid sequence that is at least 65%, at least 70%, at least 75%, at least 80%, at least 85%, at least 90%, at least 95%, at least 99%, or 100% identical to SEQ ID NO: 158; and (d) VH2 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to the amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 11; (b) VL2 comprises an amino acid sequence that is at least 65%, at least 70%, at least 75%, at least 80%, at least 85%, at least 90%, at least 95%, at least 99%, or 100% identical to SEQ ID NO: 154; (c) VH1 comprises an amino acid sequence that is at least 65%, at least 70%, at least 75%, at least 80%, at least 85%, at least 90%, at least 95%, at least 99%, or 100% identical to SEQ ID NO: 14; and (d) VH2 comprises an amino acid sequence that is at least 65%, at least 70%, at least 75%, at least 80%, at least 85%, at least 90%, at least 95%, at least 99%, or 100% identical to SEQ ID NO: 158. In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 17; (b) VL2 comprises an amino acid sequence that is at least 65%, at least 70%, at least 75%, at least 80%, at least 85%, at least 90%, at least 95%, at least 99%, or 100% identical to SEQ ID NO: 147; (c) VH1 comprises an amino acid sequence that is at least 65%, at least 70%, at least 75%, at least 80%, at least 85%, at least 90%, at least 95%, at least 99%, or 100% identical to SEQ ID NO: 21 or 22; and (d) VH2 comprises an amino acid sequence that is at least 65%, at least 70%, at least 75%, at least 80%, at least 85%, at least 90%, at least 95%, at least 99%, or 100% identical to SEQ ID NO: 151.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628, or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 67; (b) VL2 comprises an amino acid sequence that is at least 65%, at least 70%, at least 75%, at least 80%, at least 85%, at least 90%, at least 95%, at least 99%, or 100% identical to SEQ ID NO: 147; (c) VH1 comprises an amino acid sequence that is at least 65%, at least 70%, at least 75%, at least 80%, at least 85%, at least 90%, at least 95%, at least 99%, or 100% identical to SEQ ID NO: 71 or 626; and (d) VH2 comprises an amino acid sequence that is at least 65%, at least 70%, at least 75%, at least 80%, at least 85%, at least 90%, at least 95%, at least 99%, or 100% identical to SEQ ID NO: 151.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 147; (b) VL2 comprises an amino acid sequence that is at least 65%, at least 70%, at least 75%, at least 80%, at least 85%, at least 90%, at least 95%, at least 99%, or 100% identical to SEQ ID NO: 154; (c) VH1 comprises an amino acid sequence that is at least 65%, at least 70%, at least 75%, at least 80%, at least 85%, at least 90%, at least 95%, at least 99%, or 100% identical to SEQ ID NO: 151; and (d) VH2 comprises an amino acid sequence that is at least 65%, at least 70%, at least 75%, at least 80%, at least 85%, at least 90%, at least 95%, at least 99%, or 100% identical to SEQ ID NO: 158.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 147; (b) VL2 comprises an amino acid sequence that is at least 65%, at least 70%, at least 75%, at least 80%, at least 85%, at least 90%, at least 95%, at least 99%, or 100% identical to SEQ ID NO: 174; (c) VH1 comprises an amino acid sequence that is at least 65%, at least 70%, at least 75%, at least 80%, at least 85%, at least 90%, at least 95%, at least 99%, or 100% identical to SEQ ID NO: 151; and (d) VH2 comprises an amino acid sequence that is at least 65%, at least 70%, at least 75%, at least 80%, at least 85%, at least 90%, at least 95%, at least 99%, or 100% identical to SEQ ID NO: 178.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 140; (b) VL2 comprises an amino acid sequence that is at least 65%, at least 70%, at least 75%, at least 80%, at least 85%, at least 90%, at least 95%, at least 99%, or 100% identical to SEQ ID NO: 154; (c) VH1 comprises an amino acid sequence that is at least 65%, at least 70%, at least 75%, at least 80%, at least 85%, at least 90%, at least 95%, at least 99%, or 100% identical to SEQ ID NO: 144; and (d) VH2 comprises an amino acid sequence that is at least 65%, at least 70%, at least 75%, at least 80%, at least 85%, at least 90%, at least 95%, at least 99%, or 100% identical to SEQ ID NO: 158.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 147; (b) VL2 comprises an amino acid sequence that is at least 65%, at least 70%, at least 75%, at least 80%, at least 85%, at least 90%, at least 95%, at least 99%, or 100% identical to SEQ ID NO: 147; (c) VH1 comprises an amino acid sequence that is at least 65%, at least 70%, at least 75%, at least 80%, at least 85%, at least 90%, at least 95%, at least 99%, or 100% identical to SEQ ID NO: 151; and (d) VH2 comprises an amino acid sequence that is at least 65%, at least 70%, at least 75%, at least 80%, at least 85%, at least 90%, at least 95%, at least 99%, or 100% identical to SEQ ID NO: 151.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 154; (b) VL2 comprises an amino acid sequence that is at least 65%, at least 70%, at least 75%, at least 80%, at least 85%, at least 90%, at least 95%, at least 99%, or 100% identical to SEQ ID NO: 154; (c) VH1 comprises an amino acid sequence that is at least 65%, at least 70%, at least 75%, at least 80%, at least 85%, at least 90%, at least 95%, at least 99%, or 100% identical to SEQ ID NO: 158; and (d) VH2 comprises an amino acid sequence that is at least 65%, at least 70%, at least 75%, at least 80%, at least 85%, at least 90%, at least 95%, at least 99%, or 100% identical to SEQ ID NO: 158.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 11; (b) VL2 comprises an amino acid sequence that is at least 65%, at least 70%, at least 75%, at least 80%, at least 85%, at least 90%, at least 95%, at least 99%, or 100% identical to SEQ ID NO: 11; (c) VH1 comprises an amino acid sequence that is at least 65%, at least 70%, at least 75%, at least 80%, at least 85%, at least 90%, at least 95%, at least 99%, or 100% identical to SEQ ID NO: 14; and (d) VH2 comprises an amino acid sequence that is at least 65%, at least 70%, at least 75%, at least 80%, at least 85%, at least 90%, at least 95%, at least 99%, or 100% identical to SEQ ID NO: 14.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 25; (b) VL2 comprises an amino acid sequence that is at least 65%, at least 70%, at least 75%, at least 80%, at least 85%, at least 90%, at least 95%, at least 99%, or 100% identical to SEQ ID NO: 17; (c) VH1 comprises an amino acid sequence that is at least 65%, at least 70%, at least 75%, at least 80%, at least 85%, at least 90%, at least 95%, at least 99%, or 100% identical to SEQ ID NO: 29; and (d) VH2 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 11; (b) VL2 comprises an amino acid sequence that is at least 65%, at least 70%, at least 75%, at least 80%, at least 85%, at least 90%, at least 95%, at least 99%, or 100% identical to SEQ ID NO: 17; (c) VH1 comprises an amino acid sequence that is at least 65%, at least 70%, at least 75%, at least 80%, at least 85%, at least 90%, at least 95%, at least 99%, or 100% identical to SEQ ID NO: 14; and (d) VH2 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 11; (b) VL2 comprises an amino acid sequence that is at least 65%, at least 70%, at least 75%, at least 80%, at least 85%, at least 90%, at least 95%, at least 99%, or 100% identical to SEQ ID NO: 25; (c) VH1 comprises an amino acid sequence that is at least 65%, at least 70%, at least 75%, at least 80%, at least 85%, at least 90%, at least 95%, at least 99%, or 100% identical SEQ ID NO: 14; and (d) VH2 comprises an amino acid sequence that is at least 65%, at least 70%, at least 75%, at least 80%, at least 85%, at least 90%, at least 95%, at least 99%, or 100% identical to SEQ ID NO: 29.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 4; (b) VL2 comprises an amino acid sequence that is at least 65%, at least 70%, at least 75%, at least 80%, at least 85%, at least 90%, at least 95%, at least 99%, or 100% identical to SEQ ID NO: 25; (c) VH1 comprises an amino acid sequence that is at least 65%, at least 70%, at least 75%, at least 80%, at least 85%, at least 90%, at least 95%, at least 99%, or 100% identical to SEQ ID NO: 8; and (d) VH2 comprises an amino acid sequence that is at least 65%, at least 70%, at least 75%, at least 80%, at least 85%, at least 90%, at least 95%, at least 99%, or 100% identical to SEQ ID NO: 29.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 33 or 39; (b) VL2 comprises an amino acid sequence that is at least 65%, at least 70%, at least 75%, at least 80%, at least 85%, at least 90%, at least 95%, at least 99%, or 100% identical to SEQ ID NO: 17; (c) VH1 comprises an amino acid sequence that is at least 65%, at least 70%, at least 75%, at least 80%, at least 85%, at least 90%, at least 95%, at least 99%, or 100% identical to SEQ ID NO: 37; and (d) VH2 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 11; (b) VL2 comprises an amino acid sequence that is at least 65%, at least 70%, at least 75%, at least 80%, at least 85%, at least 90%, at least 95%, at least 99%, or 100% identical to SEQ ID NO: 17; (c) VH1 comprises an amino acid sequence that is at least 65%, at least 70%, at least 75%, at least 80%, at least 85%, at least 90%, at least 95%, at least 99%, or 100% identical to SEQ ID NO: 14; and (d) VH2 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 11; (b) VL2 comprises an amino acid sequence that is at least 65%, at least 70%, at least 75%, at least 80%, at least 85%, at least 90%, at least 95%, at least 99%, or 100% identical to SEQ ID NO: 25; (c) VH1 comprises an amino acid sequence that is at least 65%, at least 70%, at least 75%, at least 80%, at least 85%, at least 90%, at least 95%, at least 99%, or 100% identical to SEQ ID NO: 14; and (d) VH2 comprises an amino acid sequence that is at least 65%, at least 70%, at least 75%, at least 80%, at least 85%, at least 90%, at least 95%, at least 99%, or 100% identical to SEQ ID NO: 29.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 4; (b) VL2 comprises an amino acid sequence that is at least 65%, at least 70%, at least 75%, at least 80%, at least 85%, at least 90%, at least 95%, at least 99%, or 100% identical to SEQ ID NO: 25; (c) VH1 comprises an amino acid sequence that is at least 65%, at least 70%, at least 75%, at least 80%, at least 85%, at least 90%, at least 95%, at least 99%, or 100% identical to SEQ ID NO: 8; and (d) VH2 comprises an amino acid sequence that is at least 65%, at least 70%, at least 75%, at least 80%, at least 85%, at least 90%, at least 95%, at least 99%, or 100% identical to SEQ ID NO: 29.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 25; (b) VL2 comprises an amino acid sequence that is at least 65%, at least 70%, at least 75%, at least 80%, at least 85%, at least 90%, at least 95%, at least 99%, or 100% identical to SEQ ID NO: 17; (c) VH1 comprises an amino acid sequence that is at least 65%, at least 70%, at least 75%, at least 80%, at least 85%, at least 90%, at least 95%, at least 99%, or 100% identical to SEQ ID NO: 29; and (d) VH2 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; (b) VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (c) VH1 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40 or 327; and (d) VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 33 or 39; (b) VL2 comprises an amino acid sequence that is at least 65%, at least 70%, at least 75%, at least 80%, at least 85%, at least 90%, at least 95%, at least 99%, or 100% identical to SEQ ID NO: 17; (c) VH1 comprises an amino acid sequence that is at least 65%, at least 70%, at least 75%, at least 80%, at least 85%, at least 90%, at least 95%, at least 99%, or 100% identical to SEQ ID NO: 37; and (d) VH2 comprises an amino acid sequence that is at least 65%, at least 70%, at least 75%, at least 80%, at least 85%, at least 90%, at least 95%, at least 99%, or 100% identical to SEQ ID NO: 21 or 22.

Also provided herein is an antigen binding polypeptide having a structure, from amino-terminus to carboxy-terminus, represented by: VL1-L1-CL-L2-VH1-L3-CH1; VL1-L1-CH1-L2-VH1-L3-CL; VH1-L1-CL-L2-VL1-L3-CH1; or VH1-L1-CH1-L2-VL1-L3-CL; wherein: VL1 is an immunoglobulin light chain variable region that specifically binds to a severe acute respiratory syndrome coronavirus 2 (SARS-CoV-2) protein; VH1 is an immunoglobulin heavy chain variable region that specifically binds to a SARS-CoV-2 protein; CL is an immunoglobulin light chain constant region; CH1 is an immunoglobulin heavy chain constant region 1; and L1-L3 are optional amino acid linkers.

In some aspects, (a) VL1 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; and (b) VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (a) VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; and (b) VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884.

In some aspects, (a) VL1 comprises a CDR1 having an amino acid sequence that is at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence that is at least 65%, at least 70%, at least 75%, at least 80%, at least 85%, at least 90%, at least 95%, at least 99%, or 100% identical to amino acid sequence GAS, and a CDR3 having an amino acid sequence that is at least 65%, at least 70%, at least 75%, at least 80%, at least 85%, at least 90%, at least 95%, at least 99%, or 100% identical to SEQ ID NO: 10; and (b) VH1 comprises a CDR1 having an amino acid sequence that is at least 65%, at least 70%, at least 75%, at least 80%, at least 85%, at least 90%, at least 95%, at least 99%, or 100% identical to SEQ ID NO: 12 or 1057, a CDR2 having an amino acid sequence that is at least 65%, at least 70%, at least 75%, at least 80%, at least 85%, at least 90%, at least 95%, at least 99%, or 100% identical to SEQ ID NO: 6 or 1058, and a CDR3 having an amino acid sequence that is at least 65%, at least 70%, at least 75%, at least 80%, at least 85%, at least 90%, at least 95%, at least 99%, or 100% identical to SEQ ID NO: 13 or 1059.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 11; and (b) VH1 comprises an amino acid sequence that is at least 65%, at least 70%, at least 75%, at least 80%, at least 85%, at least 90%, at least 95%, at least 99%, or 100% identical to SEQ ID NO: 14.

Also provided herein is an antigen binding polypeptide having a structure, from amino-terminus to carboxy-terminus, represented by: VL1-L1-VH1-L2-CL; or VH1-L1-VL1-L2-CL; wherein: VL1 is an immunoglobulin light chain variable region that specifically binds to a severe acute respiratory syndrome coronavirus 2 (SARS-CoV-2) protein; VH1 is an immunoglobulin heavy chain variable region that specifically binds to a SARS-CoV-2 protein; CL is an immunoglobulin light chain constant region; and L1-L2 are optional amino acid linkers.

Also provided herein is an antigen binding polypeptide having a structure, from amino-terminus to carboxy-terminus, represented by: VL1-L1-VH1-L2-CH1; or VH1-L1-VL1-L2-CH1; wherein: VL1 is an immunoglobulin light chain variable region that specifically binds to a severe acute respiratory syndrome coronavirus 2 (SARS-CoV-2) protein; VH1 is an immunoglobulin heavy chain variable region that specifically binds to a SARS-CoV-2 protein; CH1 is an immunoglobulin heavy chain constant region 1; and L1-L2 are optional amino acid linkers.

In some aspects, (a) VL1 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; and (b) VH1 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (a) VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; and (b) VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331.626, 775, 777, 784, 791, 800, 806, 813, 820, 826, 835, 842, 848, 855, 864, 871, 877, and 884.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; and (b) VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 17; and (b) VH1 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; and (b) VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 11; and (b) VH1 comprises an amino acid sequence that is at least 65%, at least 70%, at least 75%, at least 80%, at least 85%, at least 90%, at least 95%, at least 99%, or 100% identical to SEQ ID NO: 14. In some aspects, (a) VL1 comprises a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73; and (b) VH1 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, (a) L1 comprises an amino acid sequence that is at least 65%, at least 70%, at least 75%, at least 80%, at least 85%, at least 90%, at least 95%, at least 99%, or 100% identical to SEQ ID NO: 74; and (b) VH1 comprises an amino acid sequence that is at least 65%, at least 70%, at least 75%, at least 80%, at least 85%, at least 90%, at least 95%, at least 99%, or 100% identical to SEQ ID NO: 78.

Also provided herein is an antigen binding polypeptide having a structure, from amino-terminus to carboxy-terminus, represented by: VL1-L1-VL2-L2-CH1; or VL2-L1-VL1-L2-CH1; wherein VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; CH1 is an immunoglobulin heavy chain constant region 1; and L1-L2 are optional amino acid linkers.

In some aspects, (a) VL1 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; and (b) VL2 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066.

In some aspects, (a) VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; and (b) VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988.

Also provided herein is an antigen binding polypeptide having a structure, from amino-terminus to carboxy-terminus, represented by: VH1-L1-VH2-L2-CL; or VH2-L1-VH1-L2-CL; wherein VH1 is a first immunoglobulin heavy chain variable region that specifically binds to a severe acute respiratory syndrome coronavirus 2 (SARS-CoV-2) protein; VH2 is a second immunoglobulin heavy chain variable region that specifically binds to a SARS-CoV-2 protein; CL is an immunoglobulin light chain constant region; and L1-L2 are optional amino acid linkers.

In some aspects, (a) VH1 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069; and (b) VH2 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (a) VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884; and (b) VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884.

Also provided herein is an antigen binding polypeptide having a structure, from amino-terminus to carboxy-terminus, represented by: VL1-L1-VL2-L2-VL3-L3-CH1; VL1-L1-VL3-L2-VL2-L3-CH1; VL2-L1-VL1-L2-VL3-L3-CH1; VL2-L1-VL3-L2-VL1-L3-CH1; VL3-L1-VL1-L2-VL2-L3-CH1; or VL3-L1-VL2-L2-VL1-L3-CH1; wherein VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; CH1 is an immunoglobulin heavy chain constant region 1; and L1-L3 are optional amino acid linkers.

In some aspects, (a) VL1 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; (b) VL2 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; and (c) VL3 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066.

In some aspects, (a) VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; (b) VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; and (c) VL3 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988.

Also provided herein is an antigen binding polypeptide having a structure, from amino-terminus to carboxy-terminus, represented by: VH1-L1-VH2-L2-VH3-L3-CL; VH1-L1-VH3-L2-VH2-L3-CL; VH2-L1-VH1-L2-VH3-L3-CL; VH2-L1-VH3-L2-VH1-L3-CL; VH3-L1-VH1-L2-VH2-L3-CL; or VH3-L1-VH2-L2-VH1-L3-CL; wherein VH1 is a first immunoglobulin heavy chain variable region that specifically binds to a severe acute respiratory syndrome coronavirus 2 (SARS-CoV-2) protein; VH2 is a second immunoglobulin heavy chain variable region that specifically binds to a SARS-CoV-2 protein; VH3 is a third immunoglobulin heavy chain variable region that specifically binds to a SARS-CoV-2 protein; CL is an immunoglobulin light chain constant region; and L1-L3 are optional amino acid linkers.

In some aspects, (a) VH1 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069; (b) VH2 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069; and (c) VH3 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (a) VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884; (b) VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884; and (c) VH3 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884.

In some aspects, the antigen binding polypeptide further comprises an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380. In some aspects, the Fc region comprises one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise amino acid substitutions: (i) L234A and L235A (LA); (ii) M428L and N434S (LS); (iii) L234F and L235E; (iv) L234F, L235E, and P331S (TM); and/or (v) M252Y, S254T, and T256E (YTE); or equivalent thereof, based on the EU numbering scheme.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VL2-L3-VH2-L4-VH1-L5-CH1; VL1-L1-CL-L2-VH2-L3-VL2-L4-VH1-L5-CH1; VL1-L1-CH1-L2-VL2-L3-VH2-L4-VH1-L5-CL; VL1-L1-CH1-L2-VH2-L3- VL2-L4-VH1-L5-CL; VH1-L1-CL-L2-VL2-L3-VH2-L4-VL1-L5-CH1; VH1-L1-CL-L2-VH2-L3-VL2-L4-VL1-L5-CH1; VH1-L1-CH1-L2-VL2-L3-VH2-L4-VL1-L5-CL; VH1-L1-CH1-L2-VH2-L3-VL2-L4- VL1-L5-CL; VL2-L1-CL-L2-VL1-L3-VH1-L4-VH2-L5-CH1; VL2-L1-CL-L2-VH1-L3-VL1-L4-VH2-L5-CH1; VL2-L1-CH1-L2-VL1-L3-VH1-L4-VH2-L5-CL; VL2-L1-CH1-L2-VH1-L3-VL1-L4-VH2-L5- CL; VH2-L1-CL-L2-VL1-L3-VH1-L4-VL2-L5-CH1; VH2-L1-CL-L2-VH1-L3-VL1-L4-VL2-L5-CH1; VH2-L1-CH1-L2-VL1-L3-VH1-L4-VL2-L5-CL; or VH2-L1-CH1-L2-VH1-L3-VL1-L4-VL2-L5-CL; wherein the second polypeptide has a structure, from amino-terminus to carboxy-terminus, represented by: VL3-L6-CL-L7-VL4-L8-VH4-L9-VH3-L10-CH1; VL3-L6-CL-L7-VH4-L8-VL4-L9-VH3-L10-CH1; VL3-L6- CH1-L7-VL4-L8-VH4-L9-VH3-L10-CL; VL3-L6-CH1-L7-VH4-L8-VL4-L9-VH3-L10-CL; VH3-L6-CL-L7- VL4-L8-VH4-L9-VL3-L10-CH1; VH3-L6-CL-L7-VH4-L8-VL4-L9-VL3-L10-CH1; VH3-L6-CH1-L7-VL4-L8-VH4-L9-VL3-L10-CL; VH3-L6-CH1-L7-VH4-L8-VL4-L9-VL3-L10-CL; VL4-L6-CL-L7-VL3-L8- VH3-L9-VH4-L10-CH1; VL4-L6-CL-L7-VH3-L8-VL3-L9-VH4-L10-CH1; VL4-L6-CH1-L7-VL3-L8-VH3-L9-VH4-L10-CL; VL4-L6-CH1-L7-VH3-L8-VL3-L9-VH4-L10-CL; VH4-L6-CL-L7-VL3-L8-VH3-L9- VL4-L10-CH1; VH4-L6-CL-L7-VH3-L8-VL3-L9-VL4-L10-CH1; VH4-L6-CH1-L7-VL3-L8-VH3-L9-VL4-L10-CL; or VH4-L6-CH1-L7-VH3-L8-VL3-L9-VL4-L10-CL; and wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CL is an immunoglobulin light chain constant region; CH1 is an immunoglobulin heavy chain constant region 1; and L1-L10 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-CL-L1-VL2-L2-VH2-L3-VH1-CH1; VL1-CL-L1-VH2-L2-VL2-L3-VH1-CH1; VL1-CH1-L1-VL2-L2-VH2-L3-VH1-CL; VL1-CH1-L1-VH2-L2-VL2-L3-VH1-CL; VH1-CL-L1-VL2-L2-VH2-L3-VL1-CH1; VH1-CL-L1-VH2-L2-VL2-L3-VL1-CH1; VH1-CH1-L1-VL2-L2-VH2-L3-VL1-CL; VH1-CH1-L1-VH2-L2-VL2-L3-VL1-CL; VL2-CL-L1-VL1-L2-VH1-L3-VH2-CH1; VL2-CL-L1-VH1-L2-VL1-L3-VH2-CH1; VL2-CH1-L1-VL1-L2-VH1-L3-VH2-CL; VL2-CH1-L1-VH1-L2- VL1-L3-VH2-CL; VH2-CL-L1-VL1-L2-VH1-L3-VL2-CH1; VH2-CL-L1-VH1-L2-VL1-L3-VL2-CH1; VH2-CH1-L1-VL1-L2-VH1-L3-VL2-CL; or VH2-CH1-L1-VH1-L2-VL1-L3-VL2-CL; wherein the second polypeptide has a structure, from amino-terminus to carboxy-terminus, represented by: VL3-CL-L4-VL4-L5-VH4-L6-VH3-CH1; VL3-CL-L4-VH4-L5-VL4-L6-VH3-CH1; VL3-CH1-L4-VL4-L5-VH4-L6-VH3-CL; VL3-CH1-L4-VH4-L5-VL4-L6-VH3-CL; VH3-CL-L4-VL4-L5-VH4-L6-VL3-CH1; VH3-CL-L4-VH4-L5-VL4-L6-VL3-CH1; VH3-CH1-L4-VL4-L5-VH4-L6-VL3-CL; VH3-CH1-L4-VH4-L5-VL4-L6-VL3-CL; VL4-CL-L4-VL3-L5-VH3-L6-VH4-CH1; VL4-CL-L4-VH3-L5-VL3-L6-VH4-CH1; VL4-CH1-L4-VL3-L5-VH3-L6-VH4-CL; VL4-CH1-L4-VH3-L5-VL3-L6-VH4-CL; VH4-CL-L4-VL3-L5-VH3- L6-VL4-CH1; VH4-CL-L4-VH3-L5-VL3-L6-VL4-CH1; VH4-CH1-L4-VL3-L5-VH3-L6-VL4-CL; or VH4-CH1-L4-VH3-L5-VL3-L6-VL4-CL; and wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CL is an immunoglobulin light chain constant region; CH1 is an immunoglobulin heavy chain constant region 1; and L1-L6 are amino acid linkers.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 17; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 21 or 22; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 25; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 29; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL2 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL4 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH2 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7; VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH4 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 25; VL2 comprises an amino acid sequence that is at least 65%, at least 70%, at least 75%, at least 80%, at least 85%, at least 90%, at least 95%, at least 99%, or 100% identical to SEQ ID NO: 4; VL3 comprises an amino acid sequence that is at least 65%, at least 70%, at least 75%, at least 80%, at least 85%, at least 90%, at least 95%, at least 99%, or 100% identical to SEQ ID NO: 17; VL4 comprises an amino acid sequence that is at least 65%, at least 70%, at least 75%, at least 80%, at least 85%, at least 90%, at least 95%, at least 99%, or 100% identical to SEQ ID NO: 33 or 39; VH1 comprises an amino acid sequence that is at least 65%, at least 70%, at least 75%, at least 80%, at least 85%, at least 90%, at least 95%, at least 99%, or 100% identical to SEQ ID NO: 29; VH2 comprises an amino acid sequence that is at least 65%, at least 70%, at least 75%, at least 80%, at least 85%, at least 90%, at least 95%, at least 99%, or 100% identical to SEQ ID NO: 8; VH3 comprises an amino acid sequence that is at least 65%, at least 70%, at least 75%, at least 80%, at least 85%, at least 90%, at least 95%, at least 99%, or 100% identical to SEQ ID NO: 21 or 22; and VH4 comprises an amino acid sequence that is at least 65%, at least 70%, at least 75%, at least 80%, at least 85%, at least 90%, at least 95%, at least 99%, or 100% identical to SEQ ID NO: 37.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 71 or 626; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 71 or 626; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 67; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 71 or 626; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 67; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 71 or 626; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 17; VL4 comprises an amino acid sequence that is at least 65%, at least 70%, at least 75%, at least 80%, at least 85%, at least 90%, at least 95%, at least 99%, or 100% identical to SEQ ID NO: 67; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 21 or 22; and VH4 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 154; VL4 comprises an amino acid sequence that is at least 65%, at least 70%, at least 75%, at least 80%, at least 85%, at least 90%, at least 95%, at least 99%, or 100% identical to SEQ ID NO: 67; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 158; and VH4 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VL3 comprises an amino acid sequence that is at least 65%, at least 70%, at least 75%, at least 80%, at least 85%, at least 90%, at least 95%, at least 99%, or 100% identical to SEQ ID NO: 17; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 71 or 626; VH3 comprises an amino acid sequence that is at least 65%, at least 70%, at least 75%, at least 80%, at least 85%, at least 90%, at least 95%, at least 99%, or 100% identical to SEQ ID NO: 21 or 22; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 71 or 626; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 67; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 17; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 71 or 626; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 21 or 22; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 67; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 71 or 626; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 17; VL3 comprises an amino acid sequence that is at least 65%, at least 70%, at least 75%, at least 80%, at least 85%, at least 90%, at least 95%, at least 99%, or 100% identical to SEQ ID NO: 67; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 21 or 22; VH3 comprises an amino acid sequence that is at least 65%, at least 70%, at least 75%, at least 80%, at least 85%, at least 90%, at least 95%, at least 99%, or 100% identical to SEQ ID NO: 71 or 626; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 17; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 67; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 21 or 22; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 17; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 67; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 21 or 22; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 71 or 626; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 17; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 67; VH1 comprises an amino acid sequence that is at least 65%, at least 70%, at least 75%, at least 80%, at least 85%, at least 90%, at least 95%, at least 99%, or 100% identical to SEQ ID NO: 21 or 22; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 147; VL3 comprises an amino acid sequence that is at least 65%, at least 70%, at least 75%, at least 80%, at least 85%, at least 90%, at least 95%, at least 99%, or 100% identical to SEQ ID NO: 67; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 151; VH3 comprises an amino acid sequence that is at least 65%, at least 70%, at least 75%, at least 80%, at least 85%, at least 90%, at least 95%, at least 99%, or 100% identical to SEQ ID NO: 71 or 626; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 147; VL3 comprises an amino acid sequence that is at least 65%, at least 70%, at least 75%, at least 80%, at least 85%, at least 90%, at least 95%, at least 99%, or 100% identical to SEQ ID NO: 17; VL4 comprises an amino acid sequence that is at least 65%, at least 70%, at least 75%, at least 80%, at least 85%, at least 90%, at least 95%, at least 99%, or 100% identical to SEQ ID NO: 67; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 151; VH3 comprises an amino acid sequence that is at least 65%, at least 70%, at least 75%, at least 80%, at least 85%, at least 90%, at least 95%, at least 99%, or 100% identical to SEQ ID NO: 21 or 22; and VH4 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 71 or 626; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 17; VL3 comprises an amino acid sequence that is at least 65%, at least 70%, at least 75%, at least 80%, at least 85%, at least 90%, at least 95%, at least 99%, or 100% identical to SEQ ID NO: 67; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 21 or 22; VH3 comprises an amino acid sequence that is at least 65%, at least 70%, at least 75%, at least 80%, at least 85%, at least 90%, at least 95%, at least 99%, or 100% identical to SEQ ID NO: 71 or 626; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 71 or 626; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 154; VL3 comprises an amino acid sequence that is at least 65%, at least 70%, at least 75%, at least 80%, at least 85%, at least 90%, at least 95%, at least 99%, or 100% identical to SEQ ID NO: 67; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 158; VH3 comprises an amino acid sequence that is at least 65%, at least 70%, at least 75%, at least 80%, at least 85%, at least 90%, at least 95%, at least 99%, or 100% identical to SEQ ID NO: 71 or 626; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 154; VL2 comprises an amino acid sequence that is at least 65%, at least 70%, at least 75%, at least 80%, at least 85%, at least 90%, at least 95%, at least 99%, or 100% identical to SEQ ID NO: 147; VL3 comprises an amino acid sequence that is at least 65%, at least 70%, at least 75%, at least 80%, at least 85%, at least 90%, at least 95%, at least 99%, or 100% identical to SEQ ID NO: 154; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 158; VH2 comprises an amino acid sequence that is at least 65%, at least 70%, at least 75%, at least 80%, at least 85%, at least 90%, at least 95%, at least 99%, or 100% identical to SEQ ID NO: 151; VH3 comprises an amino acid sequence that is at least 65%, at least 70%, at least 75%, at least 80%, at least 85%, at least 90%, at least 95%, at least 99%, or 100% identical to SEQ ID NO: 158; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 147; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 151; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 147; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 151; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 147; VL3 comprises an amino acid sequence that is at least 65%, at least 70%, at least 75%, at least 80%, at least 85%, at least 90%, at least 95%, at least 99%, or 100% identical to SEQ ID NO: 154; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 151; VH3 comprises an amino acid sequence that is at least 65%, at least 70%, at least 75%, at least 80%, at least 85%, at least 90%, at least 95%, at least 99%, or 100% identical to SEQ ID NO: 158; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 154; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 158; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL3 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064; VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH3 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 174; VL2 comprises an amino acid sequence that is at least 65%, at least 70%, at least 75%, at least 80%, at least 85%, at least 90%, at least 95%, at least 99%, or 100% identical to SEQ ID NO: 147; VL3 comprises an amino acid sequence that is at least 65%, at least 70%, at least 75%, at least 80%, at least 85%, at least 90%, at least 95%, at least 99%, or 100% identical to SEQ ID NO: 174; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 178; VH2 comprises an amino acid sequence that is at least 65%, at least 70%, at least 75%, at least 80%, at least 85%, at least 90%, at least 95%, at least 99%, or 100% identical to SEQ ID NO: 151; VH3 comprises an amino acid sequence that is at least 65%, at least 70%, at least 75%, at least 80%, at least 85%, at least 90%, at least 95%, at least 99%, or 100% identical to SEQ ID NO: 178; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064; VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 174; VL2 comprises an amino acid sequence that is at least 65%, at least 70%, at least 75%, at least 80%, at least 85%, at least 90%, at least 95%, at least 99%, or 100% identical to SEQ ID NO: 147; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 178; VH2 comprises an amino acid sequence that is at least 65%, at least 70%, at least 75%, at least 80%, at least 85%, at least 90%, at least 95%, at least 99%, or 100% identical to SEQ ID NO: 151; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064; VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 174; VL3 comprises an amino acid sequence that is at least 65%, at least 70%, at least 75%, at least 80%, at least 85%, at least 90%, at least 95%, at least 99%, or 100% identical to SEQ ID NO: 67; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 178; VH3 comprises an amino acid sequence that is at least 65%, at least 70%, at least 75%, at least 80%, at least 85%, at least 90%, at least 95%, at least 99%, or 100% identical to SEQ ID NO: 71 or 626; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; VL3 comprises a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064; VH3 comprises a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 174; VL3 comprises an amino acid sequence that is at least 65%, at least 70%, at least 75%, at least 80%, at least 85%, at least 90%, at least 95%, at least 99%, or 100% identical to SEQ ID NO: 140; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 178; VH3 comprises an amino acid sequence that is at least 65%, at least 70%, at least 75%, at least 80%, at least 85%, at least 90%, at least 95%, at least 99%, or 100% identical to SEQ ID NO: 144; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 140; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 144; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 147; VL2 comprises an amino acid sequence that is at least 65%, at least 70%, at least 75%, at least 80%, at least 85%, at least 90%, at least 95%, at least 99%, or 100% identical to SEQ ID NO: 147; VL3 comprises an amino acid sequence that is at least 65%, at least 70%, at least 75%, at least 80%, at least 85%, at least 90%, at least 95%, at least 99%, or 100% identical to SEQ ID NO: 147; VL4 comprises an amino acid sequence that is at least 65%, at least 70%, at least 75%, at least 80%, at least 85%, at least 90%, at least 95%, at least 99%, or 100% identical to SEQ ID NO: 147; VH1 comprises an amino acid sequence that is at least 65%, at least 70%, at least 75%, at least 80%, at least 85%, at least 90%, at least 95%, at least 99%, or 100% identical to SEQ ID NO: 151; VH2 comprises an amino acid sequence that is at least 65%, at least 70%, at least 75%, at least 80%, at least 85%, at least 90%, at least 95%, at least 99%, or 100% identical to SEQ ID NO: 151; VH3 comprises an amino acid sequence that is at least 65%, at least 70%, at least 75%, at least 80%, at least 85%, at least 90%, at least 95%, at least 99%, or 100% identical to SEQ ID NO: 151; and VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; VH1 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 154; VL2 comprises an amino acid sequence that is at least 65%, at least 70%, at least 75%, at least 80%, at least 85%, at least 90%, at least 95%, at least 99%, or 100% identical to SEQ ID NO: 154; VL3 comprises an amino acid sequence that is at least 65%, at least 70%, at least 75%, at least 80%, at least 85%, at least 90%, at least 95%, at least 99%, or 100% identical to SEQ ID NO: 154; VL4 comprises an amino acid sequence that is at least 65%, at least 70%, at least 75%, at least 80%, at least 85%, at least 90%, at least 95%, at least 99%, or 100% identical to SEQ ID NO: 154; VH1 comprises an amino acid sequence that is at least 65%, at least 70%, at least 75%, at least 80%, at least 85%, at least 90%, at least 95%, at least 99%, or 100% identical to SEQ ID NO: 158; VH2 comprises an amino acid sequence that is at least 65%, at least 70%, at least 75%, at least 80%, at least 85%, at least 90%, at least 95%, at least 99%, or 100% identical to SEQ ID NO: 158; VH3 comprises an amino acid sequence that is at least 65%, at least 70%, at least 75%, at least 80%, at least 85%, at least 90%, at least 95%, at least 99%, or 100% identical to SEQ ID NO: 158; and VH4 comprises an amino acid sequence that is at least 65%, at least 70%, at least 75%, at least 80%, at least 85%, at least 90%, at least 95%, at least 99%, or 100% identical to SEQ ID NO: 158.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 11; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 14.

Also provided herein is an antigen binding polypeptide complex comprising a first polypeptide and a second polypeptide, wherein the first polypeptide has a structure, from amino-terminus to carboxy-terminus, represented by: VL1-L1-VH1-L2-VL2-L3-CL-L4-VH2-L5-CH1; VL1-L1-VH1-L2-VL2-L3-CH1-L4-VH2-L5-CL; VL1-L1-VH1-L2-VH2-L3-CL-L4-VL2-L5-CH1; VL1-L1-VH1-L2-VH2-L3-CH1-L4-VL2-L5-CL; VL1-L1-VL2-L2-VH1-L3-CL-L4-VH2-L5-CH1; VL1-L1-VL2-L2-VH1-L3-CH1-L4-VH2-L5-CL; VL1-L1-VH2-L2-VH1-L3-CL-L4-VL2-L5-CH1; VL1-L1-VH2-L2-VH1-L3-CH1-L4-VL2-L5-CL; VH1-L1-VL1-L2-VL2-L3-CL-L4-VH2-L5-CH1; VH1-L1-VL1-L2-VL2-L3-CH1-L4-VH2-L5-CL; VH1-L1-VL1-L2-VH2-L3-CL-L4-VL2-L5-CH1; VH1-L1-VL1-L2-VH2-L3-CH1-L4-VL2-L5- CL; VH1-L1-VL2-L2-VL1-L3-CL-L4-VH2-L5-CH1; VH1-L1-VL2-L2-VL1-L3-CH1-L4-VH2-L5-CL; VH1-L1-VH2-L2-VL1-L3-CL-L4-VL2-L5-CH1; VH1-L1-VH2-L2-VL1-L3-CH1-L4-VL2-L5-CL; VL2-L1-VL1-L2-VH2-L3-CL-L4-VH1-L5-CH1; VL2-L1-VL1-L2-VH2-L3-CH1-L4-VH1-L5-CL; VL2-L1-VH1-L2-VH2-L3-CL-L4-VL1-L5-CH1; VL2-L1-VH1-L2-VH2-L3-CH1-L4-VL1-L5-CL; VL2-L1-VH2-L2-VL1-L3-CL-L4-VH1-L5-CH1; VL2-L1-VH2-L2-VL1-L3-CH1-L4-VH1-L5-CL; VL2-L1-VH2-L2-VH1-L3-CL-L4-VL1-L5-CH1; VL2-L1-VH2-L2-VH1-L3-CH1-L4-VL1-L5-CL; VH2-L1-VL1-L2-VL2-L3-CL-L4-VH1-L5-CH1; VH2-L1-VL1-L2-VL2-L3-CH1-L4-VH1-L5-CL; VH2-L1-VH1-L2-VL2-L3-CL-L4-VL1-L5-CH1; VH2-L1-VH1-L2-VL2-L3-CH1-L4-VL1-L5-CL; VH2-L1-VL2-L2-VL1-L3-CL-L4-VH1-L5-CH1; VH2-L1-VL2-L2-VL1-L3-CH1-L4-VH1-L5-CL; VH2-L1-VL2-L2-VH1-L3-CL-L4-VL1-L5-CH1; or VH2-L1-VL2-L2-VH1-L3-CH1-L4-VL1-L5-CL; wherein the second polypeptide has a structure, from amino-terminus to carboxy-terminus, represented by: VL3-L6-VH3-L7-VL4-L8-CL-L9-VH4-L10-CH1; VL3-L6-VH3-L7-VL4-L8-CH1-L9-VH4-L10-CL; VL3-L6-VH3-L7-VH4-L8-CL-L9-VL4-L10-CH1; VL3-L6-VH3-L7-VH4-L8-CH1-L9-VL4-L10-CL; VL3-L6-VL4-L7-VH3-L8-CL-L9-VH4-L10-CH1; VL3-L6-VL4-L7-VH3-L8-CH1-L9-VH4-L10-CL; VL3-L6-VH4-L7-VH3-L8-CL-L9-VL4-L10-CH1; VL3-L6-VH4-L7-VH3-L8-CH1-L9-VL4-L10-CL; VH3-L6-VL3-L7-VL4-L8-CL-L9-VH4-L10-CH1; VH3-L6-VL3-L7-VL4-L8-CH1-L9-VH4-L10-CL; VH3-L6-VL3-L7-VH4-L8-CL-L9-VL4-L10-CH1; VH3-L6-VL3-L7-VH4-L8-CH1-L9-VL4-L10-CL; VH3-L6-VL4-L7-VL3-L8-CL-L9-VH4-L10-CH1; VH3-L6-VL4-L7-VL3-L8-CH1-L9-VH4-L10-CL; VH3-L6-VH4-L7-VL3-L8-CL-L9-VL4-L10-CH1; VH3-L6-VH4-L7-VL3-L8-CH1-L9-VL4-L10-CL; VL4-L6-VL3-L7-VH4-L8-CL-L9-VH3-L10-CH1; VL4-L6-VL3-L7-VH4-L8-CH1-L9-VH3-L10-CL; VL4-L6-VH3-L7-VH4-L8-CL-L9-VL3-L10-CH1; VL4-L6-VH3-L7-VH4-L8-CH1-L9-VL3-L10-CL; VL4-L6-VH4-L7-VL3-L8-CL-L9-VH3-L10-CH1; VL4-L6-VH4-L7-VL3-L8-CH1-L9-VH3-L10-CL; VL4-L6-VH4-L7-VH3-L8-CL-L9-VL3-L10-CH1; VL4-L6-VH4-L7-VH3-L8-CH1-L9-VL3-L10-CL; VH4-L6-VL3-L7-VL4-L8-CL-L9-VH3-L10-CH1; VH4-L6-VL3-L7-VL4-L8-CH1-L9-VH3-L10-CL; VH4-L6-VH3-L7-VL4-L8-CL-L9-VL3-L10-CH1; VH4-L6-VH3-L7-VL4-L8-CH1-L9-VL3-L10-CL; VH4-L6-VL4-L7-VL3-L8-CL-L9-VH3-L10-CH1; VH4-L6-VL4-L7-VL3-L8-CH1-L9-VH3-L10-CL; VH4-L6-VL4-L7-VH3-L8-CL-L9-VL3-L10-CH1; or VH4-L6-VL4-L7-VH3-L8-CH1-L9-VL3-L10-CL; and wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CL is an immunoglobulin light chain constant region; CH1 is an immunoglobulin heavy chain constant region 1; and L1-L10 are optional amino acid linkers.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 17; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 21 or 22; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 25; VL3 comprises an amino acid sequence that is at least 65%, at least 70%, at least 75%, at least 80%, at least 85%, at least 90%, at least 95%, at least 99%, or 100% identical to SEQ ID NO: 17; VL4 comprises an amino acid sequence that is at least 65%, at least 70%, at least 75%, at least 80%, at least 85%, at least 90%, at least 95%, at least 99%, or 100% identical to SEQ ID NO: 11; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 29; VH3 comprises an amino acid sequence that is at least 65%, at least 70%, at least 75%, at least 80%, at least 85%, at least 90%, at least 95%, at least 99%, or 100% identical to SEQ ID NO: 21 or 22; and VH4 comprises an amino acid sequence that is at least 65%, at least 70%, at least 75%, at least 80%, at least 85%, at least 90%, at least 95%, at least 99%, or 100% identical to SEQ ID NO: 14.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL3 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7; VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH3 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 4; VL2 comprises an amino acid sequence that is at least 65%, at least 70%, at least 75%, at least 80%, at least 85%, at least 90%, at least 95%, at least 99%, or 100% identical to SEQ ID NO: 25; VL3 comprises an amino acid sequence that is at least 65%, at least 70%, at least 75%, at least 80%, at least 85%, at least 90%, at least 95%, at least 99%, or 100% identical to SEQ ID NO: 33 or 39; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 8; VH2 comprises an amino acid sequence that is at least 65%, at least 70%, at least 75%, at least 80%, at least 85%, at least 90%, at least 95%, at least 99%, or 100% identical to SEQ ID NO: 29; VH3 comprises an amino acid sequence that is at least 65%, at least 70%, at least 75%, at least 80%, at least 85%, at least 90%, at least 95%, at least 99%, or 100% identical to SEQ ID NO: 37; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VH1-L3-CH1-L4-VL2-L5-VH2; VL1-L1-CL-L2-VH1-L3-CH1-L4-VH2-L5-VL2; VL1-L1-CL-L2-VL2-L3-CH1-L4-VH1-L5-VH2; VL1-L1-CL-L2-VH2-L3-CH1-L4-VH1-L5-VL2; VL1-L1-CH1-L2-VH1-L3-CL-L4-VL2-L5-VH2; VL1-L1-CH1-L2-VH1-L3-CL-L4-VH2-L5-VL2; VL1-L1-CH1-L2-VL2-L3-CL-L4-VH1-L5-VH2; VL1-L1-CH1-L2-VH2-L3-CL-L4-VH1-L5-VL2; VH1-L1-CL-L2-VL1-L3-CH1-L4-VL2-L5-VH2; VH1-L1-CL-L2-VL1-L3-CH1-L4-VH2-L5-VL2; VH1-L1-CL-L2-VL2-L3-CH1-L4-VL1-L5-VH2; VH1-L1-CL-L2-VH2-L3-CH1-L4-VL1-L5-VL2; VH1-L1-CH1-L2-VL1-L3-CL-L4-VL2-L5-VH2; VH1-L1-CH1-L2-VL1-L3-CL-L4-VH2-L5-VL2; VH1-L1-CH1-L2-VL2-L3-CL-L4-VL1-L5-VH2; VH1-L1-CH1-L2-VH2-L3-CL-L4-VL1-L5-VL2; VL2-L1-CL-L2-VL1-L3-CH1-L4-VH2-L5-VH1; VL2-L1-CL-L2-VH1-L3-CH1-L4-VH2-L5-VL1; VL2-L1-CL-L2-VH2-L3-CH1-L4-VL1-L5-VH1; VL2-L1-CL-L2-VH2-L3-CH1-L4-VH1-L5-VL1; VL2-L1-CH1-L2- VL1-L3-CL-L4-VH2-L5-VH1; VL2-L1-CH1-L2-VH1-L3-CL-L4-VH2-L5-VL1; VL2-L1-CH1-L2-VH2-L3-CL-L4-VL1-L5-VH1; VL2-L1-CH1-L2-VH2-L3-CL-L4-VH1-L5-VL1; VH2-L1-CL-L2-VL1-L3-CH1- L4-VL2-L5-VH1; VH2-L1-CL-L2-VH1-L3-CH1-L4-VL2-L5-VL1; VH2-L1-CL-L2-VL2-L3-CH1-L4-VL1-L5-VH1; VH2-L1-CL-L2-VL2-L3-CH1-L4-VH1-L5-VL1; VH2-L1-CH1-L2-VL1-L3-CL-L4-VL2-L5-VH1; VH2-L1-CH1-L2-VH1-L3-CL-L4-VL2-L5-VL1; VH2-L1-CH1-L2-VL2-L3-CL-L4-VL1-L5-VH1; or VH2-L1-CH1-L2-VL2-L3-CL-L4-VH1-L5-VL1; wherein the second polypeptide has a structure, from amino-terminus to carboxy-terminus, represented by: VL3-L6-CL-L7-VH3-L8-CH1-L9-VL4-L10-VH4; VL3-L6-CL-L7-VH3-L8-CH1-L9-VH4-L10-VL4; VL3-L6-CL-L7-VL4-L8-CH1-L9-VH3-L10-VH4; VL3-L6-CL-L7-VH4-L8-CH1-L9-VH3-L10-VL4; VL3-L6-CH1-L7-VH3-L8-CL-L9-VL4-L10-VH4; VL3-L6-CH1-L7-VH3-L8-CL-L9-VH4-L10-VL4; VL3-L6-CH1-L7-VL4-L8-CL-L9-VH3-L10-VH4; VL3-L6-CH1-L7-VH4-L8-CL-L9-VH3-L10-VL4; VH3-L6-CL-L7-VL3-L8-CH1-L9-VL4-L10-VH4; VH3-L6-CL-L7-VL3-L8-CH1-L9-VH4-L10-VL4; VH3-L6-CL-L7-VL4-L8-CH1-L9-VL3-L10-VH4; VH3-L6-CL-L7-VH4-L8-CH1-L9-VL3-L10-VL4; VH3-L6-CH1-L7-VL3-L8-CL-L9-VL4-L10-VH4; VH3-L6-CH1-L7-VL3-L8-CL-L9-VH4-L10-VL4; VH3-L6-CH1-L7-VL4-L8-CL-L9-VL3-L10-VH4; VH3-L6-CH1-L7-VH4-L8-CL-L9-VL3-L10-VL4; VL4-L6-CL-L7-VL3-L8-CH1-L9-VH4-L10-VH3; VL4-L6-CL-L7-VH3-L8-CH1-L9-VH4-L10-VL3; VL4-L6-CL-L7-VH4-L8-CH1-L9-VL3-L10-VH3; VL4-L6-CL-L7-VH4-L8-CH1-L9-VH3-L10-VL3; VL4-L6-CH1-L7-VL3-L8-CL-L9-VH4-L10-VH3; VL4-L6-CH1-L7-VH3-L8-CL-L9-VH4-L10-VL3; VL4-L6-CH1-L7-VH4-L8-CL-L9-VL3-L10-VH3; VL4-L6-CH1-L7-VH4-L8-CL-L9-VH3-L10-VL3; VH4-L6-CL-L7-VL3-L8-CH1-L9-VL4-L10-VH3; VH4-L6-CL-L7-VH3-L8-CH1-L9-VL4-L10-VL3; VH4-L6-CL-L7-VL4-L8-CH1-L9-VL3-L10-VH3; VH4-L6-CL-L7-VL4-L8-CH1-L9-VH3-L10-VL3; VH4-L6-CH1-L7-VL3-L8-CL-L9-VL4-L10-VH3; VH4-L6-CH1-L7-VH3-L8-CL-L9-VL4-L10-VL3; VH4-L6-CH1-L7-VL4-L8-CL-L9-VL3-L10-VH3; or VH4-L6-CH1-L7-VL4-L8-CL-L9-VH3-L10-VL3; and wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CL is an immunoglobulin light chain constant region; CH1 is an immunoglobulin heavy chain constant region 1; and L1-L10 are optional amino acid linkers.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 17; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 21 or 22; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 25; VL3 comprises an amino acid sequence that is at least 65%, at least 70%, at least 75%, at least 80%, at least 85%, at least 90%, at least 95%, at least 99%, or 100% identical to SEQ ID NO: 17; VL4 comprises an amino acid sequence that is at least 65%, at least 70%, at least 75%, at least 80%, at least 85%, at least 90%, at least 95%, at least 99%, or 100% identical to SEQ ID NO: 11; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 29; VH3 comprises an amino acid sequence that is at least 65%, at least 70%, at least 75%, at least 80%, at least 85%, at least 90%, at least 95%, at least 99%, or 100% identical to SEQ ID NO: 21 or 22; and VH4 comprises an amino acid sequence that is at least 65%, at least 70%, at least 75%, at least 80%, at least 85%, at least 90%, at least 95%, at least 99%, or 100% identical to SEQ ID NO: 14.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL3 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7; VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH3 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 4; VL2 comprises an amino acid sequence that is at least 65%, at least 70%, at least 75%, at least 80%, at least 85%, at least 90%, at least 95%, at least 99%, or 100% identical to SEQ ID NO: 25; VL3 comprises an amino acid sequence that is at least 65%, at least 70%, at least 75%, at least 80%, at least 85%, at least 90%, at least 95%, at least 99%, or 100% identical to SEQ ID NO: 33, or 39; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 8; VH2 comprises an amino acid sequence that is at least 65%, at least 70%, at least 75%, at least 80%, at least 85%, at least 90%, at least 95%, at least 99%, or 100% identical to SEQ ID NO: 29; VH3 comprises an amino acid sequence that is at least 65%, at least 70%, at least 75%, at least 80%, at least 85%, at least 90%, at least 95%, at least 99%, or 100% identical to SEQ ID NO: 37; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

Also provided herein is an antigen binding polypeptide complex comprising a first polypeptide and a second polypeptide, wherein the first polypeptide has a structure, from amino-terminus to carboxy-terminus, represented by: VL1-L1-VH1-L2-VL2-L3-CL-L4-VH2-L5-CH1; VL1-L1-VH1-L2-VL2-L3-CH1-L4-VH2-L5-CL; VL1-L1-VH1-L2-VH2-L3-CL-L4-VL2-L5-CH1; VL1-L1-VH1-L2-VH2-L3-CH1-L4-VL2-L5-CL; VL1-L1-VL2-L2-VH1-L3-CL-L4-VH2-L5-CH1; VL1-L1-VL2-L2-VH1-L3-CH1-L4-VH2-L5-CL; VL1-L1-VH2-L2-VH1-L3-CL-L4-VL2-L5-CH1; VL1-L1-VH2-L2-VH1-L3-CH1-L4-VL2-L5-CL; VH1-L1-VL1-L2-VL2-L3-CL-L4-VH2-L5-CH1; VH1-L1-VL1-L2-VL2-L3-CH1-L4-VH2-L5-CL; VH1-L1-VL1-L2-VH2-L3-CL-L4-VL2-L5-CH1; VH1-L1-VL1-L2-VH2-L3-CH1-L4-VL2-L5- CL; VH1-L1-VL2-L2-VL1-L3-CL-L4-VH2-L5-CH1; VH1-L1-VL2-L2-VL1-L3-CH1-L4-VH2-L5-CL; VH1-L1-VH2-L2-VL1-L3-CL-L4-VL2-L5-CH1; VH1-L1-VH2-L2-VL1-L3-CH1-L4-VL2-L5-CL; VL2-L1-VL1-L2-VH2-L3-CL-L4-VH1-L5-CH1; VL2-L1-VL1-L2-VH2-L3-CH1-L4-VH1-L5-CL; VL2-L1-VH1-L2-VH2-L3-CL-L4-VL1-L5-CH1; VL2-L1-VH1-L2-VH2-L3-CH1-L4-VL1-L5-CL; VL2-L1-VH2-L2-VL1-L3-CL-L4-VH1-L5-CH1; VL2-L1-VH2-L2-VL1-L3-CH1-L4-VH1-L5-CL; VL2-L1-VH2-L2-VH1-L3-CL-L4-VL1-L5-CH1; VL2-L1-VH2-L2-VH1-L3-CH1-L4-VL1-L5-CL; VH2-L1-VL1-L2-VL2-L3-CL-L4-VH1-L5-CH1; VH2-L1-VL1-L2-VL2-L3-CH1-L4-VH1-L5-CL; VH2-L1-VH1-L2-VL2-L3-CL-L4-VL1-L5-CH1; VH2-L1-VH1-L2-VL2-L3-CH1-L4-VL1-L5-CL; VH2-L1-VL2-L2-VL1-L3-CL-L4-VH1-L5-CH1; VH2-L1-VL2-L2-VL1-L3-CH1-L4-VH1-L5-CL; VH2-L1-VL2-L2-VH1-L3-CL-L4-VL1-L5-CH1; or VH2-L1-VL2-L2-VH1-L3-CH1-L4-VL1-L5-CL; wherein the second polypeptide has a structure, from amino-terminus to carboxy-terminus, represented by: VL3-L6-CL-L7-VH3-L8-CH1-L9-VL4-L10-VH4; VL3-L6-CL-L7-VH3-L8-CH1-L9-VH4-L10-VL4; VL3-L6-CL-L7-VL4-L8-CH1-L9-VH3-L10-VH4; VL3-L6-CL-L7-VH4-L8-CH1-L9-VH3-L10-VL4; VL3-L6-CH1-L7-VH3-L8-CL-L9-VL4-L10-VH4; VL3-L6-CH1-L7-VH3-L8-CL-L9-VH4-L10-VL4; VL3-L6-CH1-L7-VL4-L8-CL-L9-VH3-L10-VH4; VL3-L6-CH1-L7-VH4-L8-CL-L9-VH3-L10-VL4; VH3-L6-CL-L7-VL3-L8-CH1-L9-VL4-L10-VH4; VH3-L6-CL-L7-VL3-L8-CH1-L9-VH4-L10-VL4; VH3-L6-CL-L7-VL4-L8-CH1-L9-VL3-L10-VH4; VH3-L6-CL-L7-VH4-L8-CH1-L9-VL3-L10-VL4; VH3-L6-CH1-L7-VL3-L8-CL-L9-VL4-L10-VH4; VH3-L6-CH1-L7-VL3-L8-CL-L9-VH4-L10-VL4; VH3-L6-CH1-L7-VL4-L8-CL-L9-VL3-L10-VH4; VH3-L6-CH1-L7-VH4-L8-CL-L9-VL3-L10-VL4; VL4-L6-CL-L7-VL3-L8-CH1-L9-VH4-L10-VH3; VL4-L6-CL-L7-VH3-L8-CH1-L9-VH4-L10-VL3; VL4-L6-CL-L7-VH4-L8-CH1-L9-VL3-L10-VH3; VL4-L6-CL-L7-VH4-L8-CH1-L9-VH3-L10-VL3; VL4-L6-CH1-L7-VL3-L8-CL-L9-VH4-L10-VH3; VL4-L6-CH1-L7-VH3-L8-CL-L9-VH4-L10-VL3; VL4-L6-CH1-L7-VH4-L8-CL-L9-VL3-L10-VH3; VL4-L6-CH1-L7-VH4-L8-CL-L9-VH3-L10-VL3; VH4-L6-CL-L7-VL3-L8-CH1-L9-VL4-L10-VH3; VH4-L6-CL-L7-VH3-L8-CH1-L9-VL4-L10-VL3; VH4-L6-CL-L7-VL4-L8-CH1-L9-VL3-L10-VH3; VH4-L6-CL-L7-VL4-L8-CH1-L9-VH3-L10-VL3; VH4-L6-CH1-L7-VL3-L8-CL-L9-VL4-L10-VH3; VH4-L6-CH1-L7-VH3-L8-CL-L9-VL4-L10-VL3; VH4-L6-CH1-L7-VL4-L8-CL-L9-VL3-L10-VH3; or VH4-L6-CH1-L7-VL4-L8-CL-L9-VH3-L10-VL3; and wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CL is an immunoglobulin light chain constant region; CH1 is an immunoglobulin heavy chain constant region 1; and L1-L10 are optional amino acid linkers.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 17; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 21 or 22; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 25; VL3 comprises an amino acid sequence that is at least 65%, at least 70%, at least 75%, at least 80%, at least 85%, at least 90%, at least 95%, at least 99%, or 100% identical to SEQ ID NO: 17; VL4 comprises an amino acid sequence that is at least 65%, at least 70%, at least 75%, at least 80%, at least 85%, at least 90%, at least 95%, at least 99%, or 100% identical to SEQ ID NO: 11; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 29; VH3 comprises an amino acid sequence that is at least 65%, at least 70%, at least 75%, at least 80%, at least 85%, at least 90%, at least 95%, at least 99%, or 100% identical to SEQ ID NO: 21 or 22; and VH4 comprises an amino acid sequence that is at least 65%, at least 70%, at least 75%, at least 80%, at least 85%, at least 90%, at least 95%, at least 99%, or 100% identical to SEQ ID NO: 14.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL3 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7; VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH3 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 4; VL2 comprises an amino acid sequence that is at least 65%, at least 70%, at least 75%, at least 80%, at least 85%, at least 90%, at least 95%, at least 99%, or 100% identical to SEQ ID NO: 25; VL3 comprises an amino acid sequence that is at least 65%, at least 70%, at least 75%, at least 80%, at least 85%, at least 90%, at least 95%, at least 99%, or 100% identical to SEQ ID NO: 33 or 39; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 8; VH2 comprises an amino acid sequence that is at least 65%, at least 70%, at least 75%, at least 80%, at least 85%, at least 90%, at least 95%, at least 99%, or 100% identical to SEQ ID NO: 29; VH3 comprises an amino acid sequence that is at least 65%, at least 70%, at least 75%, at least 80%, at least 85%, at least 90%, at least 95%, at least 99%, or 100% identical to SEQ ID NO: 37; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VH1-L3-CH1-L4-VL2-L5-VH2; VL1-L1-CL-L2-VH1-L3-CH1-L4-VH2-L5-VL2; VL1-L1-CL-L2-VL2-L3-CH1-L4-VH1-L5-VH2; VL1-L1-CL-L2-VH2-L3-CH1-L4-VH1-L5-VL2; VL1-L1-CH1-L2-VH1-L3-CL-L4-VL2-L5-VH2; VL1-L1-CH1-L2-VH1-L3-CL-L4-VH2-L5-VL2; VL1-L1-CH1-L2-VL2-L3-CL-L4-VH1-L5-VH2; VL1-L1-CH1-L2-VH2-L3-CL-L4-VH1-L5-VL2; VH1-L1-CL-L2-VL1-L3-CH1-L4-VL2-L5-VH2; VH1-L1-CL-L2-VL1-L3-CH1-L4-VH2-L5-VL2; VH1-L1-CL-L2-VL2-L3-CH1-L4-VL1-L5-VH2; VH1-L1-CL-L2-VH2-L3-CH1-L4-VL1-L5-VL2; VH1-L1-CH1-L2-VL1-L3-CL-L4-VL2-L5-VH2; VH1-L1-CH1-L2-VL1-L3-CL-L4-VH2-L5-VL2; VH1-L1-CH1-L2-VL2-L3-CL-L4-VL1-L5-VH2; VH1-L1-CH1-L2-VH2-L3-CL-L4-VL1-L5-VL2; VL2-L1-CL-L2-VL1-L3-CH1-L4-VH2-L5-VH1; VL2-L1-CL-L2-VH1-L3-CH1-L4-VH2-L5-VL1; VL2-L1-CL-L2-VH2-L3-CH1-L4-VL1-L5-VH1; VL2-L1-CL-L2-VH2-L3-CH1-L4-VH1-L5-VL1; VL2-L1-CH1-L2- VL1-L3-CL-L4-VH2-L5-VH1; VL2-L1-CH1-L2-VH1-L3-CL-L4-VH2-L5-VL1; VL2-L1-CH1-L2-VH2-L3-CL-L4-VL1-L5-VH1; VL2-L1-CH1-L2-VH2-L3-CL-L4-VH1-L5-VL1; VH2-L1-CL-L2-VL1-L3-CH1- L4-VL2-L5-VH1; VH2-L1-CL-L2-VH1-L3-CH1-L4-VL2-L5-VL1; VH2-L1-CL-L2-VL2-L3-CH1-L4-VL1-L5-VH1; VH2-L1-CL-L2-VL2-L3-CH1-L4-VH1-L5-VL1; VH2-L1-CH1-L2-VL1-L3-CL-L4-VL2-L5-VH1; VH2-L1-CH1-L2-VH1-L3-CL-L4-VL2-L5-VL1; VH2-L1-CH1-L2-VL2-L3-CL-L4-VL1-L5-VH1; or VH2-L1-CH1-L2-VL2-L3-CL-L4-VH1-L5-VL1; wherein the second polypeptide has a structure, from amino-terminus to carboxy-terminus, represented by: VL3-L6-VH3-L7-VL4-L8-CL-L9-VH4-L10-CH1; VL3-L6-VH3-L7-VL4-L8-CH1-L9-VH4-L10-CL; VL3-L6-VH3-L7-VH4-L8-CL-L9-VL4-L10-CH1; VL3-L6-VH3-L7-VH4-L8-CH1-L9-VL4-L10-CL; VL3-L6-VL4-L7-VH3-L8-CL-L9-VH4-L10-CH1; VL3-L6-VL4-L7-VH3-L8-CH1-L9-VH4-L10-CL; VL3-L6-VH4-L7-VH3-L8-CL-L9-VL4-L10-CH1; VL3-L6-VH4-L7-VH3-L8-CH1-L9-VL4-L10-CL; VH3-L6-VL3-L7-VL4-L8-CL-L9-VH4-L10-CH1; VH3-L6-VL3-L7-VL4-L8-CH1-L9-VH4-L10-CL; VH3-L6-VL3-L7-VH4-L8-CL-L9-VL4-L10-CH1; VH3-L6-VL3-L7-VH4-L8-CH1-L9-VL4-L10-CL; VH3-L6-VL4-L7-VL3-L8-CL-L9-VH4-L10-CH1; VH3-L6-VL4-L7-VL3-L8-CH1-L9-VH4-L10-CL; VH3-L6-VH4-L7-VL3-L8-CL-L9-VL4-L10-CH1; VH3-L6-VH4-L7-VL3-L8-CH1-L9-VL4-L10-CL; VL4-L6-VL3-L7-VH4-L8-CL-L9-VH3-L10-CH1; VL4-L6-VL3-L7-VH4-L8-CH1-L9-VH3-L10-CL; VL4-L6-VH3-L7-VH4-L8-CL-L9-VL3-L10-CH1; VL4-L6-VH3-L7-VH4-L8-CH1-L9-VL3-L10-CL; VL4-L6-VH4-L7-VL3-L8-CL-L9-VH3-L10-CH1; VL4-L6-VH4-L7-VL3-L8-CH1-L9-VH3-L10-CL; VL4-L6-VH4-L7-VH3-L8-CL-L9-VL3-L10-CH1; VL4-L6-VH4-L7-VH3-L8-CH1-L9-VL3-L10-CL; VH4-L6-VL3-L7-VL4-L8-CL-L9-VH3-L10-CH1; VH4-L6-VL3-L7-VL4-L8-CH1-L9-VH3-L10-CL; VH4-L6-VH3-L7-VL4-L8-CL-L9-VL3-L10-CH1; VH4-L6-VH3-L7-VL4-L8-CH1-L9-VL3-L10-CL; VH4-L6-VL4-L7-VL3-L8-CL-L9-VH3-L10-CH1; VH4-L6-VL4-L7-VL3-L8-CH1-L9-VH3-L10-CL; VH4-L6-VL4-L7-VH3-L8-CL-L9-VL3-L10-CH1; or VH4-L6-VL4-L7-VH3-L8-CH1-L9-VL3-L10-CL; and wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CL is an immunoglobulin light chain constant region; CH1 is an immunoglobulin heavy chain constant region 1; and L1-L10 are optional amino acid linkers.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 17; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 21 or 22; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22. In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL3 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7; VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH3 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327; and VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 4; VL2 comprises an amino acid sequence that is at least 65%, at least 70%, at least 75%, at least 80%, at least 85%, at least 90%, at least 95%, at least 99%, or 100% identical to SEQ ID NO: 25; VL3 comprises an amino acid sequence that is at least 65%, at least 70%, at least 75%, at least 80%, at least 85%, at least 90%, at least 95%, at least 99%, or 100% identical to SEQ ID NO: 33 or 39; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 8; VH2 comprises an amino acid sequence that is at least 65%, at least 70%, at least 75%, at least 80%, at least 85%, at least 90%, at least 95%, at least 99%, or 100% identical to SEQ ID NO: 29; VH3 comprises an amino acid sequence that is at least 65%, at least 70%, at least 75%, at least 80%, at least 85%, at least 90%, at least 95%, at least 99%, or 100% identical to SEQ ID NO: 37; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, the first polypeptide and the second polypeptide each further comprise an Fc region at the carboxy-terminus. In some aspects, the Fc region of the first polypeptide and the second polypeptide each comprise a CH2 and/or CH3. In some aspects, the Fc region of the first polypeptide and the second polypeptide each further comprise an immunoglobulin hinge. In some aspects, the Fc region of the first polypeptide and/or the Fc region of the second polypeptide comprise an amino acid sequence that is at least 65%, at least 70%, at least 75%, at least 80%, at least 85%, at least 90%, at least 95%, at least 99%, or 100% identical to any one of SEQ ID NOs: 376-380. In some aspects, the Fc region of the first polypeptide and/or the Fc region of the second polypeptide comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise amino acid substitutions: (i) L234A and L235A (LA); (ii) M428L and N434S (LS); (iii) L234F and L235E; (iv) L234F, L235E, and P331S (TM); and/or (v) M252Y, S254T, and T256E (YTE); or equivalent thereof, based on the EU numbering scheme.

Also provided herein is an antigen binding polypeptide having a structure, from amino-terminus to carboxy-terminus, represented by: VL1-L1-VL2-L2-VH2-L3-VH1-L4-CL-L5-CH1; VL1-L1-VL2-L2-VH2-L3-VH1-L4-CH1-L5-CL; VL1-L1-VH2-L2-VL2-L3-VH1-L4-CL-L5-CH1; VL1-L1-VH2-L2-VL2-L3-VH1-L4-CH1-L5-CL; VH1-L1-VL2-L2-VH2-L3-VL1-L4-CL-L5-CH1; VH1-L1-VL2-L2-VH2-L3-VL1-L4-CH1-L5-CL; VH1-L1-VH2-L2-VL2-L3-VL1-L4-CL-L5-CH1; VH1-L1-VH2-L2-VL2-L3-VL1-L4-CH1-L5-CL; VL2-L1-VL1-L2-VH1-L3-VH2-L4-CL-L5-CH1; VL2-L1-VL1-L2-VH1-L3-VH2-L4-CH1-L5-CL; VL2-L1-VH1-L2-VL1-L3-VH2-L4-CL-L5-CH1; VL2-L1-VH1-L2-VL1-L3-VH2-L4-CH1-L5-CL; VH2-L1-VL1-L2-VH1-L3-VL2-L4-CL-L5-CH1; VH2-L1-VL1-L2-VH1-L3-VL2-L4-CH1-L5-CL; VH2-L1-VH1-L2-VL1-L3-VL2-L4-CL-L5-CH1; or VH2-L1-VH1-L2-VL1-L3-VL2-L4-CH1-L5-CL; wherein: (a) VL1 is a first immunoglobulin light chain variable region that specifically binds to a severe acute respiratory syndrome coronavirus 2 (SARS-CoV-2) protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; or wherein; (b) VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; or wherein: (c) VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH2 is a second immunoglobulin heavy chain variable region that specifically binds to a SARS-CoV-2 protein, comprising; and a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and wherein: CL is an immunoglobulin light chain constant region; CH1 is an immunoglobulin heavy chain constant region 1; and L1-L5 are optional amino acid linkers.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 17; VL2 comprises an amino acid sequence that is at least 65%, at least 70%, at least 75%, at least 80%, at least 85%, at least 90%, at least 95%, at least 99%, or 100% identical to SEQ ID NO: 11; VH1 comprises an amino acid sequence that is at least 65%, at least 70%, at least 75%, at least 80%, at least 85%, at least 90%, at least 95%, at least 99%, or 100% identical to SEQ ID NO: 21 or 22; and VH2 comprises an amino acid sequence that is at least 65%, at least 70%, at least 75%, at least 80%, at least 85%, at least 90%, at least 95%, at least 99%, or 100% identical to SEQ ID NO: 14; (b) VL1 comprises an amino acid sequence that is at least 65%, at least 70%, at least 75%, at least 80%, at least 85%, at least 90%, at least 95%, at least 99%, or 100% identical to SEQ ID NO: 25; VL2 comprises an amino acid sequence that is at least 65%, at least 70%, at least 75%, at least 80%, at least 85%, at least 90%, at least 95%, at least 99%, or 100% identical to SEQ ID NO: 11; VH1 comprises an amino acid sequence that is at least 65%, at least 70%, at least 75%, at least 80%, at least 85%, at least 90%, at least 95%, at least 99%, or 100% identical to SEQ ID NO: 29; and VH2 comprises an amino acid sequence that is at least 65%, at least 70%, at least 75%, at least 80%, at least 85%, at least 90%, at least 95%, at least 99%, or 100% identical to SEQ ID NO: 14; or (c) VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VH1 comprises an amino acid sequence that is at least 65%, at least 70%, at least 75%, at least 80%, at least 85%, at least 90%, at least 95%, at least 99%, or 100% identical to SEQ ID NO: 14; and VH2 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 17; VL2 comprises an amino acid sequence that is at least 65%, at least 70%, at least 75%, at least 80%, at least 85%, at least 90%, at least 95%, at least 99%, or 100% identical to SEQ ID NO: 11; VH1 comprises an amino acid sequence that is at least 65%, at least 70%, at least 75%, at least 80%, at least 85%, at least 90%, at least 95%, at least 99%, or 100% identical to SEQ ID NO: 21 or 22; and VH2 comprises an amino acid sequence that is at least 65%, at least 70%, at least 75%, at least 80%, at least 85%, at least 90%, at least 95%, at least 99%, or 100% identical to SEQ ID NO: 14.

In some aspects, VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 25; VL2 comprises an amino acid sequence that is at least 65%, at least 70%, at least 75%, at least 80%, at least 85%, at least 90%, at least 95%, at least 99%, or 100% identical to SEQ ID NO: 11; VH1 comprises an amino acid sequence that is at least 65%, at least 70%, at least 75%, at least 80%, at least 85%, at least 90%, at least 95%, at least 99%, or 100% identical to SEQ ID NO: 29; and VH2 comprises an amino acid sequence that is at least 65%, at least 70%, at least 75%, at least 80%, at least 85%, at least 90%, at least 95%, at least 99%, or 100% identical to SEQ ID NO: 14.

In some aspects, VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VH1 comprises an amino acid sequence that is at least 65%, at least 70%, at least 75%, at least 80%, at least 85%, at least 90%, at least 95%, at least 99%, or 100% identical to SEQ ID NO: 14; and VH2 comprises an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptide further comprises an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380. In some aspects, Fc region comprises one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise amino acid substitutions: (i) L234A and L235A (LA); (ii) M428L and N434S (LS); (iii) L234F and L235E; (iv) L234F, L235E, and P331S (TM); and/or (v) M252Y, S254T, and T256E (YTE); or equivalent thereof, based on the EU numbering scheme.

Also provided herein is an antigen binding polypeptide complex comprising a first polypeptide and a second polypeptide, wherein the first polypeptide has a structure, from amino-terminus to carboxy-terminus, represented by: VL1-L1-VL2-L2-VH2-L3-VH1-L4-CL-L5-CH1; VL1-L1-VL2-L2-VH2-L3-VH1-L4-CH1-L5-CL; VL1-L1-VH2-L2-VL2-L3-VH1-L4-CL-L5-CH1; VL1-L1-VH2-L2-VL2-L3-VH1-L4-CH1-L5-CL; VH1-L1-VL2-L2-VH2-L3-VL1-L4-CL-L5-CH1; VH1-L1-VL2-L2-VH2-L3-VL1-L4-CH1-L5-CL; VH1-L1-VH2-L2-VL2-L3-VL1-L4-CL-L5-CH1; VH1-L1-VH2-L2-VL2-L3-VL1-L4-CH1-L5-CL; VL2-L1-VL1-L2-VH1-L3-VH2-L4-CL-L5-CH1; VL2-L1-VL1-L2-VH1-L3-VH2-L4-CH1-L5-CL; VL2-L1-VH1-L2-VL1-L3-VH2-L4-CL-L5-CH1; VL2-L1-VH1-L2-VL1-L3-VH2-L4-CH1-L5- CL; VH2-L1-VL1-L2-VH1-L3-VL2-L4-CL-L5-CH1; VH2-L1-VL1-L2-VH1-L3-VL2-L4-CH1-L5-CL; VH2-L1-VH1-L2-VL1-L3-VL2-L4-CL-L5-CH1; or VH2-L1-VH1-L2-VL1-L3-VL2-L4-CH1-L5-CL; wherein the second polypeptide has a structure, from amino-terminus to carboxy-terminus, represented by: VL3-L6-VL4-L7-VH4-L8-VH3-L9-CL-L10-CH1; VL3-L6-VL4-L7-VH4-L8-VH3-L9-CH1-L10-CL; VL3-L6-VH4-L7-VL4-L8-VH3-L9-CL-L10-CH1; VL3-L6-VH4-L7-VL4-L8-VH3-L9-CH1-L10-CL; VH3-L6-VL4-L7-VH4-L8-VL3-L9-CL-L10-CH1; VH3-L6-VL4-L7-VH4-L8-VL3-L9-CH1-L10-CL; VH3-L6-VH4-L7-VL4-L8-VL3-L9-CL-L10-CH1; VH3-L6-VH4-L7-VL4-L8-VL3-L9-CH1-L10-CL; VL4-L6-VL3-L7-VH3-L8-VH4-L9-CL-L10-CH1; VL4-L6-VL3-L7-VH3-L8-VH4-L9-CH1-L10-CL; VL4-L6-VH3-L7-VL3-L8-VH4-L9-CL-L10-CH1; VL4-L6-VH3-L7-VL3-L8-VH4-L9-CH1-L10-CL; VH4-L6-VL3-L7-VH3-L8-VL4-L9-CL-L10-CH1; VH4-L6-VL3-L7-VH3-L8-VL4-L9-CH1-L10-CL; VH4-L6-VH3-L7-VL3-L8-VL4-L9-CL-L10-CH1; or VH4-L6-VH3-L7-VL3-L8-VL4-L9-CH1-L10-CL; and wherein: (a) VL1 is a first immunoglobulin light chain variable region that specifically binds to a severe acute respiratory syndrome coronavirus 2 (SARS-CoV-2) protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 is a third immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 is a fourth immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 is a third immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH4 is a fourth immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; or wherein; (b) VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 is a third immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 is a fourth immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 is a third immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 is a fourth immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; or wherein: (c) VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL3 is a third immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 is a fourth immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH3 is a third immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 is a fourth immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and wherein: CL is an immunoglobulin light chain constant region; CH1 is an immunoglobulin heavy chain constant region 1; and L1-L10 are optional amino acid linkers.

In some aspects, (a) VL1 comprises an amino acid sequence that is at least 65%, at least 70%, at least 75%, at least 80%, at least 85%, at least 90%, at least 95%, at least 99%, or 100% identical to SEQ ID NO: 17; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 21 or 22; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22; or (b) VL1 comprises an amino acid sequence that is at least 65%, at least 70%, at least 75%, at least 80%, at least 85%, at least 90%, at least 95%, at least 99%, or 100% identical to SEQ ID NO: 25; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 29; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22; or (c) VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 71 or 626; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 is a third immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 is a fourth immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 is a third immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH4 is a fourth immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 17; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 21 or 22; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL3 is a third immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 is a fourth immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH3 is a third immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 is a fourth immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 25; VL2 comprises an amino acid sequence that is at least 65%, at least 70%, at least 75%, at least 80%, at least 85%, at least 90%, at least 95%, at least 99%, or 100% identical to SEQ ID NO: 11; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 29; VH2 comprises an amino acid sequence that is at least 65%, at least 70%, at least 75%, at least 80%, at least 85%, at least 90%, at least 95%, at least 99%, or 100% identical to SEQ ID NO: 14; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 is a first immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 is a second immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; VL3 is a third immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL4 is a fourth immunoglobulin light chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 is a first immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 is a second immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; VH3 is a third immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and VH4 is a fourth immunoglobulin heavy chain variable region that specifically binds to a SARS-CoV-2 protein, comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 67; VL3 comprises an amino acid sequence that is at least 65%, at least 70%, at least 75%, at least 80%, at least 85%, at least 90%, at least 95%, at least 99%, or 100% identical to SEQ ID NO: 11; VL4 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 71 or 626; VH3 comprises an amino acid sequence that is at least 65%, at least 70%, at least 75%, at least 80%, at least 85%, at least 90%, at least 95%, at least 99%, or 100% identical to SEQ ID NO: 14; and VH4 comprises an amino acid sequence that is at least 65%, at least 70%, at least 75%, at least 80%, at least 85%, at least 90%, at least 95%, at least 99%, or 100% identical to SEQ ID NO: 21 or 22.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VH1-L3-CH1; VL1-L1-CH1-L2-VH1-L3-CL; VH1-L1-CL-L2-VL1-L3-CH1; or VH1-L1-CH1-L2-VL1-L3-CL; wherein the second polypeptide has a structure, from amino-terminus to carboxy-terminus, represented by: VL2-L4-CL-L5-VL3-L6-VH3-L7-VH2-L8-CH1; VL2-L4-CL-L5-VH3-L6-VL3-L7-VH2-L8-CH1; VL2-L4-CH1-L5-VL3-L6-VH3-L7-VH2-L8- CL; VL2-L4-CH1-L5-VH3-L6-VL3-L7-VH2-L8-CL; VH2-L4-CL-L5-VL3-L6-VH3-L7-VL2-L8-CH1; VH2-L4-CL-L5-VH3-L6-VL3-L7-VL2-L8-CH1; VH2-L4-CH1-L5-VL3-L6-VH3-L7-VL2-L8-CL; VH2-L4-CH1-L5-VH3-L6-VL3-L7-VL2-L8-CL; VL3-L4-CL-L5-VL2-L6-VH2-L7-VH3-L8-CH1; VL3-L4-CL-L5-VH2-L6-VL2-L7-VH3-L8-CH1; VL3-L4-CH1-L5-VL2-L6-VH2-L7-VH3-L8-CL; VL3-L4-CH1-L5- VH2-L6-VL2-L7-VH3-L8-CL; VH3-L4-CL-L5-VL2-L6-VH2-L7-VL3-L8-CH1; VH3-L4-CL-L5-VH2-L6-VL2-L7-VL3-L8-CH1; VH3-L4-CH1-L5-VL2-L6-VH2-L7-VL3-L8-CL; or VH3-L4-CH1-L5-VH2-L6-VL2-L7-VL3-L8-CL; and wherein: VL1 is a first immunoglobulin light chain variable region that specifically binds a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; CL is an immunoglobulin light chain constant region; CH1 is an immunoglobulin heavy chain constant region 1; and L1-L8 are optional amino acid linkers.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL3 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH3 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 17; VL3 comprises an amino acid sequence that is at least 65%, at least 70%, at least 75%, at least 80%, at least 85%, at least 90%, at least 95%, at least 99%, or 100% identical to SEQ ID NO: 25; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 21 or 22; and VH3 comprises an amino acid sequence that is at least 65%, at least 70%, at least 75%, at least 80%, at least 85%, at least 90%, at least 95%, at least 99%, or 100% identical to SEQ ID NO: 29.

Also provided herein is an antigen binding polypeptide complex comprising a first polypeptide and a second polypeptide, herein the first polypeptide has a structure, from amino-terminus to carboxy-terminus, represented by: VL1-L1-CL-L2-VH1-L3-CH1; VL1-L1-CH1-L2-VH1-L3-CL; VH1-L1-CL-L2-VL1-L3-CH1; or VH1-L1-CH1-L2-VL1-L3-CL; wherein the second polypeptide has a structure, from amino-terminus to carboxy-terminus, represented by: VL2-L4-VH2-L5-VL3-L6-CL-L7-VH3-L8-CH1; VL2-L4-VH2-L5-VL3-L6-CH1-L7-VH3-L8-CL; VL2-L4-VH2-L5-VH3-L6-CL-L7-VL3-L8-CH1; VL2-L4-VH2-L5-VH3-L6-CH1-L7-VL3-L8-CL; VL2-L4-VL3-L5-VH2-L6-CL-L7-VH3-L8-CH1; VL2-L4-VL3-L5-VH2-L6-CH1-L7-VH3-L8-CL; VL2-L4-VH3-L5-VH2-L6-CL-L7-VL3-L8-CH1; VL2-L4-VH3-L5-VH2-L6-CH1-L7-VL3-L8-CL; VH2-L4-VL2-L5-VL3-L6-CL-L7-VH3-L8-CH1; VH2-L4-VL2-L5-VL3-L6-CH1-L7-VH3-L8-CL; VH2-L4-VL2-L5-VH3-L6-CL-L7-VL3-L8-CH1; VH2-L4-VL2-L5-VH3-L6-CH1-L7-VL3-L8-CL; VH2-L4-VL3-L5-VL2-L6-CL-L7-VH3-L8-CH1; VH2-L4-VL3-L5-VL2-L6-CH1-L7-VH3-L8-CL; VH2-L4-VH3-L5-VL2-L6-CL-L7-VL3-L8-CH1; VH2-L4-VH3-L5-VL2-L6-CH1-L7-VL3-L8-CL; VL3-L4-VL2-L5-VH3-L6-CL-L7-VH2-L8-CH1; VL3-L4-VL2-L5-VH3-L6-CH1-L7-VH2-L8-CL; VL3-L4-VH2-L5-VH3-L6-CL-L7-VL2-L8-CH1; VL3-L4-VH2-L5-VH3-L6-CH1-L7-VL2-L8-CL; VL3-L4-VH3-L5-VL2-L6-CL-L7-VH2-L8-CH1; VL3-L4-VH3-L5-VL2-L6-CH1-L7-VH2-L8-CL; VL3-L4-VH3-L5-VH2-L6-CL-L7-VL2-L8-CH1; VL3-L4-VH3-L5-VH2-L6-CH1-L7-VL2-L8- CL; VH3-L4-VL2-L5-VL3-L6-CL-L7-VH2-L8-CH1; VH3-L4-VL2-L5-VL3-L6-CH1-L7-VH2-L8-CL; VH3-L4-VH2-L5-VL3-L6-CL-L7-VL2-L8-CH1; VH3-L4-VH2-L5-VL3-L6-CH1-L7-VL2-L8-CL; VH3-L4-VL3-L5-VL2-L6-CL-L7-VH2-L8-CH1; VH3-L4-VL3-L5-VL2-L6-CH1-L7-VH2-L8-CL; VH3-L4-VL3-L5-VH2-L6-CL-L7-VL2-L8-CH1; or VH3-L4-VL3-L5-VH2-L6-CH1-L7-VL2-L8-CL; and wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; CL is an immunoglobulin light chain constant region; CH1 is an immunoglobulin heavy chain constant region 1; and L1-L8 are optional amino acid linkers.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 25; VL3 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 29; and VH3 comprises an amino acid sequence that is at least 65%, at least 70%, at least 75%, at least 80%, at least 85%, at least 90%, at least 95%, at least 99%, or 100% identical to SEQ ID NO: 21 or 22.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VH1-L3-CH1; VL1-L1-CH1-L2-VH1-L3-CL; VH1-L1-CL-L2-VL1-L3-CH1; or VH1-L1-CH1-L2-VL1-L3-CL; wherein the second polypeptide has a structure, from amino-terminus to carboxy-terminus, represented by: VL2-L4-CL-L5-VH2-L6-CH1-L7-VL3-L8-VH3; VL2-L4-CL-L5-VH2-L6-CH1-L7-VH3-L8-VL3; VL2-L4-CL-L5-VL3-L6-CH1-L7-VH2-L8-VH3; VL2-L4-CL-L5-VH3-L6-CH1-L7-VH2-L8-VL3; VL2-L4-CH1-L5-VH2-L6-CL-L7-VL3-L8-VH3; VL2-L4-CH1-L5-VH2-L6-CL-L7-VH3-L8-VL3; VL2-L4-CH1-L5-VL3-L6-CL-L7-VH2-L8-VH3; VL2-L4-CH1-L5-VH3-L6-CL-L7-VH2-L8-VL3; VH2-L4-CL-L5-VL2-L6-CH1-L7-VL3-L8-VH3; VH2-L4-CL-L5-VL2-L6-CH1-L7-VH3-L8-VL3; VH2-L4-CL-L5-VL3-L6-CH1-L7-VL2-L8-VH3; VH2-L4-CL-L5-VH3-L6-CH1-L7-VL2-L8-VL3; VH2-L4-CH1-L5-VL2-L6-CL-L7-VL3-L8-VH3; VH2-L4-CH1-L5- VL2-L6-CL-L7-VH3-L8-VL3; VH2-L4-CH1-L5-VL3-L6-CL-L7-VL2-L8-VH3; VH2-L4-CH1-L5-VH3-L6-CL-L7-VL2-L8-VL3; VL3-L4-CL-L5-VL2-L6-CH1-L7-VH3-L8-VH2; VL3-L4-CL-L5-VH2-L6-CH1- L7-VH3-L8-VL2; VL3-L4-CL-L5-VH3-L6-CH1-L7-VL2-L8-VH2; VL3-L4-CL-L5-VH3-L6-CH1-L7-VH2-L8-VL2; VL3-L4-CH1-L5-VL2-L6-CL-L7-VH3-L8-VH2; VL3-L4-CH1-L5-VH2-L6-CL-L7-VH3-L8-VL2; VL3-L4-CH1-L5-VH3-L6-CL-L7-VL2-L8-VH2; VL3-L4-CH1-L5-VH3-L6-CL-L7-VH2-L8-VL2; VH3-L4-CL-L5-VL2-L6-CH1-L7-VL3-L8-VH2; VH3-L4-CL-L5-VH2-L6-CH1-L7-VL3-L8-VL2; VH3-L4-CL-L5-VL3-L6-CH1-L7-VL2-L8-VH2; VH3-L4-CL-L5-VL3-L6-CH1-L7-VH2-L8-VL2; VH3-L4-CH1-L5-VL2-L6-CL-L7-VL3-L8-VH2; VH3-L4-CH1-L5-VH2-L6-CL-L7-VL3-L8-VL2; VH3-L4-CH1-L5-VL3-L6-CL-L7-VL2-L8-VH2; or VH3-L4-CH1-L5-VL3-L6-CL-L7-VH2-L8-VL2; and wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; CL is an immunoglobulin light chain constant region; CH1 is an immunoglobulin heavy chain constant region 1; and L1-L8 are optional amino acid linkers.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 25; VL3 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 14; VH2 comprises an amino acid sequence that is at least 65%, at least 70%, at least 75%, at least 80%, at least 85%, at least 90%, at least 95%, at least 99%, or 100% identical to SEQ ID NO: 29; and VH3 comprises an amino acid sequence that is at least 65%, at least 70%, at least 75%, at least 80%, at least 85%, at least 90%, at least 95%, at least 99%, or 100% identical to SEQ ID NO: 21 or 22.

Also provided herein is an antigen binding polypeptide complex comprising a first polypeptide and a second polypeptide, wherein the first polypeptide has a structure, from amino-terminus to carboxy-terminus, represented by: VL1-L1-VH1-L2-CL; or VH1-L3-VL1-L4-CL; wherein the second polypeptide has a structure, from amino-terminus to carboxy-terminus, represented by: VL2-L1-VH2-L2-CH1; or VH2-L3-VL2-L4-CH1; wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CL is an immunoglobulin light chain constant region; CH1 is an immunoglobulin heavy chain constant region 1; and L1-L4 are optional amino acid linkers.

In some aspects, (a) VL1 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; (b) VH1 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069; (c) VL2 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; and (d) VH2 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (a) VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; (b) VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884; (c) VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; and (d) VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 17; VL2 comprises an amino acid sequence that is at least 65%, at least 70%, at least 75%, at least 80%, at least 85%, at least 90%, at least 95%, at least 99%, or 100% identical to SEQ ID NO: 17; VH1 comprises an amino acid sequence that is at least 65%, at least 70%, at least 75%, at least 80%, at least 85%, at least 90%, at least 95%, at least 99%, or 100% identical to SEQ ID NO: 21 or 22; and VH2 comprises an amino acid sequence that is at least 65%, at least 70%, at least 75%, at least 80%, at least 85%, at least 90%, at least 95%, at least 99%, or 100% identical to SEQ ID NO: 21 or 22.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 11; VL2 comprises an amino acid sequence that is at least 65%, at least 70%, at least 75%, at least 80%, at least 85%, at least 90%, at least 95%, at least 99%, or 100% identical to SEQ ID NO: 11; VH1 comprises an amino acid sequence that is at least 65%, at least 70%, at least 75%, at least 80%, at least 85%, at least 90%, at least 95%, at least 99%, or 100% identical to SEQ ID NO: 14; and VH2 comprises an amino acid sequence that is at least 65%, at least 70%, at least 75%, at least 80%, at least 85%, at least 90%, at least 95%, at least 99%, or 100% identical to SEQ ID NO: 14.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73; VL2 comprises a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73; VH1 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77; and VH2 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, VL1 comprises an amino acid sequence that is at least 65%, at least 70%, at least 75%, at least 80%, at least 85%, at least 90%, at least 95%, at least 99%, or 100% identical to SEQ ID NO: 74; VL2 comprises an amino acid sequence that is at least 65%, at least 70%, at least 75%, at least 80%, at least 85%, at least 90%, at least 95%, at least 99%, or 100% identical to SEQ ID NO: 74; VH1 comprises an amino acid sequence that is at least 65%, at least 70%, at least 75%, at least 80%, at least 85%, at least 90%, at least 95%, at least 99%, or 100% identical to SEQ ID NO: 78; and VH2 comprises an amino acid sequence that is at least 65%, at least 70%, at least 75%, at least 80%, at least 85%, at least 90%, at least 95%, at least 99%, or 100% identical to SEQ ID NO: 78.

Also provided herein is an antigen binding polypeptide complex comprising a first polypeptide and a second polypeptide, wherein the first polypeptide has a structure, from amino-terminus to carboxy-terminus, represented by: VL1-L1-VL2-L2-CH1; or VL2-L1-VL1-L2-CH1; wherein the second polypeptide has a structure, from amino-terminus to carboxy-terminus, represented by: VH1-L3-VH2-L4-CL; or VH2-L3-VH1-L4-CL; wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CL is an immunoglobulin light chain constant region; CH1 is an immunoglobulin heavy chain constant region 1; and L1-L4 are optional amino acid linkers.

In some aspects, (a) VL1 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; (b) VH1 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069; (c) VL2 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; and (d) VH2 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (a) VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; (b) VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884; (c) VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; and (d) VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884.

Also provided herein is an antigen binding polypeptide complex comprising a first polypeptide and a second polypeptide, wherein the first polypeptide has a structure, from amino-terminus to carboxy-terminus, represented by: VL1-L1-VL2-L2-VL3-L3-CH1; VL1-L1-VL3-L2-VL2-L3-CH1; VL2-L1-VL1-L2-VL3-L3-CH1; VL2-L1-VL3-L2-VL1-L3-CH1; VL3-L1-VL1-L2-VL2-L3-CH1; or VL3-L1-VL2-L2-VL1-L3-CH1; wherein the second polypeptide has a structure, from amino-terminus to carboxy-terminus, represented by: VH1-L4-VH2-L5-VH3-L6-CL; VH1-L4-VH3-L5-VH2-L6-CL; VH2-L4-VH1-L5-VH3-L6-CL; VH2-L4-VH3-L5-VH1-L6-CL; VH3-L4-VH1-L5-VH2-L6-CL; or VH3-L4-VH2-L5-VH1-L6- CL; wherein: VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VL3 is a third immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; CL is an immunoglobulin light chain constant region; CH1 is an immunoglobulin heavy chain constant region 1; and L1-L6 are optional amino acid linkers.

In some aspects, (a) VL1 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; (b) VH1 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069; (c) VL2 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; (d) VH2 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069; (e) VL3 comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; and (f) VH3 comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (a) VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; (b) VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884; (c) VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; (d) VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884; (e) VL3 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; and (f) VH3 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884.

In some aspects, the first polypeptide and the second polypeptide each further comprise an Fc region at the carboxy-terminus. In some aspects, the Fc region of the first polypeptide and the second polypeptide each comprise a CH2 and/or CH3. In some aspects, the Fc region of the first polypeptide and the second polypeptide each further comprise an immunoglobulin hinge. In some aspects, the Fc region of the first polypeptide and/or the Fc region of the second polypeptide comprise an amino acid sequence that is at least 65%, at least 70%, at least 75%, at least 80%, at least 85%, at least 90%, at least 95%, at least 99%, or 100% identical to any one of SEQ ID NOs: 376-380. In some aspects, the Fc region of the first polypeptide and/or the Fc region of the second polypeptide comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise amino acid substitutions: (i) L234A and L235A (LA); (ii) M428L and N434S (LS); (iii) L234F and L235E; (iv) L234F, L235E, and P331S (TM); and/or (v) M252Y, S254T, and T256E (YTE); or equivalent thereof, based on the EU numbering scheme.

Also provided herein is an antigen binding polypeptide complex comprising a first polypeptide and a second polypeptide, wherein the first polypeptide comprises an amino acid sequence having at least 65%, at least 70%, at least 75%, at least 80%, at least 85%, at least 90%, at least 95%, at least 99%, or 100% identity to any one of SEQ ID NOs: 381-502, 632-633, 720-721, 724-725, 728-729, 732-733, 736-737, 740-741, 744-745, 748-749, 752-753, 760-761, 887-926, 972-977, 984-985, 989-990, 993-994, 997-998, 1001-1002, 1005-1006, 1009-1010, 1013-1014, 1017-1018, 1021-1022, 1025-1026, 1029-1030, 1033-1034, 1037-1038, and 1041-1042; and the second polypeptide comprises an amino acid sequence having at least 65%, at least 70%, at least 75%, at least 80%, at least 85%, at least 90%, at least 95%, at least 99%, or 100% identity to any one of SEQ ID NOs: 381-502, 632-633, 720-721, 724-725, 728-729, 732-733, 736-737, 740-741, 744-745, 748-749, 752-753, 760-761, 887-926, 972-977, 984-985, 989-990, 993-994, 997-998, 1001-1002, 1005-1006, 1009-1010, 1013-1014, 1017-1018, 1021-1022, 1025-1026, 1029-1030, 1033-1034, 1037-1038, and 1041-1042.

In some aspects, the SARS-CoV-2 protein is a SARS-CoV-2 spike protein. In some aspects, any one of the optional linkers each independently have a length of from 0 amino acids to about 50 amino acids. In some aspects, any one of the optional linkers each independently have a length of from about 5 amino acids to about 40 amino acids. In some aspects, one or more of the optional linkers each independently are non-immunogenic. In some aspects, one or more of the optional linkers each independently do not contain a consensus T cell epitope. In some aspects, one or more of the optional linkers each independently comprises an amino acid sequence of G or A; or an amino acid sequence at least 65%, at least 70%, at least 75%, at least 80%, at least 85%, at least 90%, at least 95%, at least 99%, or 100% identical to an amino acid sequence of GSS or ASG; or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, one or more of the optional linkers each independently comprises an amino acid sequence at least 65%, at least 70%, at least 75%, at least 80%, at least 85%, at least 90%, at least 95%, at least 99%, or 100% identical to any one of SEQ ID NOs: 333-345 or 967-971. In some aspects, the Fc region comprises at least one knob-into-hole modification or Fc effector function knockout mutation. In some aspects, the Fc region of the first polypeptide and/or the Fc region of the second polypeptide comprise at least one knob-into-hole modification or Fc effector function knockout mutation. In some aspects, the antigen binding polypeptide or the antigen binding polypeptide complex is an IgG1 or IgG4 antibody, or antigen binding fragment thereof, and the knob-into-hole modification comprises: (i) knob substitutions of S354C and T366W; (ii) hole substitutions of Y349C, T366S, L368A and Y407V; or (iii) a combination thereof; or equivalent thereof, based on the EU numbering scheme. In some aspects, the antigen binding polypeptide complex is an IgG1 or IgG4 antibody, or antigen binding fragment thereof, and the Fc effector function knockout mutation is L234A, L235A, P239A, or equivalent thereof, or a combination thereof, based on the EU numbering scheme.

In some aspects, the antigen binding polypeptide or antigen binding polypeptide complex disclosed herein comprises a detectable label. In some aspects, the detectable label is a radioactive label, chemiluminescent label, fluorescent label, enzyme, peptide tag, or a combination thereof. In some aspects, the peptide tag is a poly histidine tag consisting of from about four to about 10 histidine residues. In some aspects, the poly histidine tag consists of about eight histidine residues.

In some aspects, the antigen binding polypeptide or antigen binding polypeptide complex disclosed herein is conjugated to an agent as an antibody-drug conjugate (ADC). In some aspects, the agent is a cytotoxic agent, immunomodulating agent, imaging agent, or therapeutic protein, or a combination thereof.

In some aspects, the antigen binding polypeptide or antigen binding polypeptide complex disclosed herein specifically binds to the SARS-CoV-2 protein with an equilibrium dissociation constant (K_{D}) of from about 10 μM to about 1 pM.

In some aspects, the antigen binding polypeptide complex disclosed herein is a class I antibody or antigen binding fragment thereof; class II antibody or antigen binding fragment thereof; class III antibody or antigen binding fragment thereof; class IV antibody or antigen binding fragment thereof; class I and II antibody or antigen binding fragment thereof; class I and III antibody or antigen binding fragment thereof; class I and IV antibody or antigen binding fragment thereof; class II and III antibody or antigen binding fragment thereof; class II and IV antibody or antigen binding fragment thereof; class III and IV antibody or antigen binding fragment thereof; class I, II and III antibody or antigen binding fragment thereof; class I, II and IV antibody or antigen binding fragment thereof; class I, III and IV antibody or antigen binding fragment thereof; class II, III and IV antibody or antigen binding fragment thereof; or class I, II, III and IV antibody or antigen binding fragment thereof.

In some aspects, the antigen binding polypeptide complex disclosed herein is bispecific, trispecific or tetraspecific and has greater neutralization potency against SARS-CoV-2 virus than a monospecific antigen binding polypeptide complex that specifically binds to one of the same antigens as the bispecific, trispecific or tetraspecific antigen binding polypeptide complex.

In some aspects, the antigen binding polypeptide complex disclosed herein is bispecific, trispecific or tetraspecific and has greater neutralization potency against SARS-CoV-2 virus than a mixture of monospecific antigen binding polypeptide complexes that specifically bind to the same antigens as the bispecific, trispecific or tetraspecific antigen binding polypeptide complex.

In some aspects, the SARS-CoV-2 virus is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an Iota variant (e.g., B.1.526), a Lambda variant (e.g., C.37), an Epsilon variant (e.g., B.1.429. B.1.427 or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1. BA.2.75, BA.2.75.2, BA.4.6, BJ.1, XBB, XBB1.5, BF.7, CH.1.1. BA.2.86, EG.5, JN.1, JD.1, EG.5.1, FL.1.5.1, HK.3, HV.1, JD.1.1, JF.1, XBB.1, XBB.1.16, XBB.1.16.6, or XBB.2.3.2), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof.

In some aspects, the Omicron variant is B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1. HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, KP.3.1.1, or a combination thereof.

In some aspects, one or more of the VH has a mutation at an N-glycosylation site. In some aspects, the mutation is at amino acid N62 of the VH region or equivalent thereof. In some aspects, the N62 mutation is N62Q or N62S, or equivalent thereof.

In some aspects, the antigen binding polypeptide complex disclosed herein has an isoelectric point (pI) of from about 7 to about 9.

Also provided herein is an antibody or antigen binding fragment thereof comprising the antigen binding polypeptide or antigen binding polypeptide complex disclosed herein. In some aspects, the antibody or antigen binding fragment thereof is IgG, IgM, IgE, IgA or IgD. In some aspects, the IgG is IgG1, IgG2, IgG3 or IgG4. In some aspects, the antigen binding fragment is a Fab, scFab, Fab′, F(ab′)₂, Fv or scFv. In some aspects, the antibody or antigen binding fragment thereof is human or humanized.

Also provided herein is a polypeptide having at least 65%, at least 70%, at least 75%, at least 80%, at least 85%, at least 90%, at least 95%, at least 99%, or 100% identity to any one of SEQ ID NOs: 381-502, 632-633, 720-721, 724-725, 728-729, 732-733, 736-737, 740-741, 744-745, 748-749, 752-753, 760-761, 887-926, 972-977, 984-985, 989-990, 993-994, 997-998, 1001-1002, 1005-1006, 1009-1010, 1013-1014, 1017-1018, 1021-1022, 1025-1026, 1029-1030, 1033-1034, 1037-1038, and 1041-1042.

Also provided herein is a polynucleotide encoding the antigen binding polypeptide or antigen binding polypeptide complex, the antibody or antigen binding fragment thereof, or the polypeptide disclosed herein. In some aspects, the polynucleotide comprises a nucleotide sequence having at least 65%, at least 70%, at least 75%, at least 80%, at least 85%, at least 90%, at least 95%, at least 99%, or 100% identity to a nucleotide sequence of any one of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044.

Also provided herein is a polynucleotide having at least 65%, at least 70%, at least 75%, at least 80%, at least 85%, at least 90%, at least 95%, at least 99%, or 100% identity to any one of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044.

Also provided herein is a vector comprising the polynucleotide disclosed herein.

Also provided herein is a host cell comprising the vector disclosed herein.

Also provided herein is an mRNA encoding the antigen binding polypeptide or antigen binding polypeptide complex, the antibody or antigen binding fragment thereof, or the polypeptide disclosed herein. In some aspects, the mRNA comprises a 5′-cap and/or a poly (A) tail.

Also provided herein is a lipid nanoparticle (LNP) comprising the mRNA disclosed herein.

Also provided herein is a chimeric antigen receptor (CAR) comprising the antigen binding polypeptide or antigen binding polypeptide complex disclosed herein.

Also provided herein is an immune cell comprising the CAR disclosed herein.

Also provided herein is a pharmaceutical composition comprising the antigen binding polypeptide or antigen binding polypeptide complex, the antibody or antigen binding fragment thereof, the polypeptide, the polynucleotide, the vector, the host cell, the mRNA, the LNP, the CAR, or the immune cell disclosed herein.

Also provided herein is a pharmaceutical composition comprising (i) the antigen binding polypeptide or antigen binding polypeptide complex, the antibody or antigen binding fragment thereof, the polypeptide, the polynucleotide, the vector, the host cell, the mRNA, the LNP, the CAR, or the immune cell disclosed herein, and (ii) a pharmaceutically acceptable carrier.

Also provided herein is a pharmaceutical composition comprising (i) the antigen binding polypeptide or antigen binding polypeptide complex, the antibody or antigen binding fragment thereof, the polypeptide, the polynucleotide, the vector, the host cell, the mRNA, the LNP, the CAR, or the immune cell disclosed herein, and (ii) an additional pharmaceutical agent.

Also provided herein is a kit comprising (i) the antigen binding polypeptide or antigen binding polypeptide complex of any one, the antibody or antigen binding fragment thereof, the polypeptide, the polynucleotide, the vector, the host cell, the mRNA, the LNP, the CAR, the immune cell, or the pharmaceutical composition disclosed herein, and (ii) instructions for use.

Also provided herein is a method of preventing or treating a SARS-CoV-2 viral infection in a subject in need thereof, comprising administering to the subject a therapeutically effective amount of the antigen binding polypeptide or antigen binding polypeptide complex, the antibody or antigen binding fragment thereof, the polypeptide, the polynucleotide, the vector, the host cell, the mRNA, the LNP, the CAR, the immune cell, or the pharmaceutical composition disclosed herein.

Also provided herein is a method of preventing or treating coronavirus disease 2019 (COVID-19) in a subject in need thereof, comprising administering to the subject a therapeutically effective amount of the antigen binding polypeptide or antigen binding polypeptide complex, the antibody or antigen binding fragment thereof, the polypeptide, the polynucleotide, the vector, the host cell, the mRNA, the, the CAR, the immune cell, or the pharmaceutical composition disclosed herein.

Also provided herein is a method of diagnosing a subject as being infected with a SARS-CoV-2 virus or suspected of being infected with a SARS-CoV-2 virus, comprising (i) contacting a sample obtained from the subject with the antigen binding polypeptide or antigen binding polypeptide complex or the antibody or antigen binding fragment thereof disclosed herein; (ii) detecting the presence or absence of a virus complex which contains the polypeptide or a fragment thereof, polypeptide complex or a fragment thereof, antibody or a fragment thereof, and a SARS-CoV-2 virus, virion or fragment thereof; and (iii) diagnosing the subject as being infected with a SARS-CoV-2 virus or suspected of being infected with a SARS-CoV-2 virus when the presence of the virus complex is detected.

Also provided herein is a method of diagnosing a subject as not being infected with a SARS-CoV-2 virus or not suspected of being infected with a SARS-CoV-2 virus, comprising (i) contacting a sample obtained from the subject with the antigen binding polypeptide or antigen binding polypeptide complex or the antibody or antigen binding fragment thereof disclosed herein; (ii) detecting the presence or absence of a virus complex which contains the polypeptide or a fragment thereof, polypeptide complex or a fragment thereof, antibody or a fragment thereof, and a SARS-CoV-2 virus, virion or fragment thereof; and (iii) diagnosing the subject as not being infected with a SARS-CoV-2 virus or not suspected of being infected with a SARS-CoV-2 virus when the presence of the virus complex is not detected.

Also provided herein is a method of diagnosing a subject as having COVID-19 or suspected of having COVID-19, comprising (i) contacting a sample obtained from the subject with the antigen binding polypeptide or antigen binding polypeptide complex or the antibody or antigen binding fragment thereof disclosed herein; (ii) detecting the presence or absence of a virus complex which contains the polypeptide or a fragment thereof, polypeptide complex or a fragment thereof, antibody or a fragment thereof, and a SARS-CoV-2 virus, virion or fragment thereof; and (iii) diagnosing the subject as having COVID-19 or suspected of having COVID-19 when the presence of the virus complex is detected.

Also provided herein is a method of diagnosing a subject as not having COVID-19 or not suspected of having COVID-19, comprising (i) contacting a sample obtained from the subject with the antigen binding polypeptide or antigen binding polypeptide complex or the antibody or antigen binding fragment thereof disclosed herein; (ii) detecting the presence or absence of a virus complex which contains the polypeptide or a fragment thereof, polypeptide complex or a fragment thereof, antibody or a fragment thereof, and a SARS-CoV-2 virus, virion or fragment thereof; and (iii) diagnosing the subject as not having COVID-19 or not suspected of having COVID-19 when the presence of the virus complex is not detected.

In some aspects, the sample is a nasal swab, tissue sample, saliva, plasma or blood.

In some aspects, detecting the presence or absence of the virus complex comprises an enzyme linked immunosorbent assay (ELISA), immunospot assay, lateral flow assay, flow cytometry, immunohistochemistry or western blot.

Also provided herein is an anti-SARS-CoV-2 virus antibody or an antigen binding fragment thereof, wherein the anti-SARS-CoV-2 virus antibody or an antigen binding fragment thereof comprises: (a) one or more immunoglobulin light chain constant domain (CL) and (b) one or more immunoglobulin heavy chain 1 constant domain (CH1); and wherein the anti-SARS-CoV-2 virus antibody or an antigen binding fragment thereof (i) has an isoelectric point (pI), and after administered to a subject has (ii) a higher peak serum levels, and (iii) a reduced clearance levels, as compared to a same antibody or an antigen binding fragment thereof not comprising one or more immunoglobulin light chain constant domain (CL) and (b) one or more immunoglobulin heavy chain 1 constant domain (CH1).

In some aspects, the anti-SARS-CoV-2 virus antibody or an antigen binding fragment thereof is IgG, IgM, IgE, IgA or IgD. In some aspects, the anti-SARS-CoV-2 virus antibody or an antigen binding fragment thereof is IgG1, IgG2, IgG3 or IgG4. In some aspects, the anti-SARS-CoV-2 virus antibody or an antigen binding fragment thereof is a Fab, scFab, Fab′, F(ab′)₂, Fv or scFv. In some aspects, the anti-SARS-CoV-2 virus antibody or an antigen binding fragment thereof is human or humanized. In some aspects, the anti-SARS-CoV-2 virus antibody or an antigen binding fragment thereof has an isoelectric point (pI) of from about 7 to about 9). In some aspects, the anti-SARS-CoV-2 virus antibody or an antigen binding fragment thereof binds to the SARS-CoV-2 protein with an equilibrium dissociation constant (K_{D})) of from about 10 μM to about 1 pM. In some aspects, the anti-SARS-CoV-2 virus antibody or an antigen binding fragment thereof is a class I antibody or antigen binding fragment thereof; class II antibody or antigen binding fragment thereof; class III antibody or antigen binding fragment thereof; class IV antibody or antigen binding fragment thereof; class I and II antibody or antigen binding fragment thereof; class I and III antibody or antigen binding fragment thereof; class I and IV antibody or antigen binding fragment thereof; class II and III antibody (or antigen binding fragment thereof; class II and IV antibody or antigen binding fragment thereof; class III and IV antibody or antigen binding fragment thereof; class I, II and III antibody or antigen binding fragment thereof; class I, II and IV antibody or antigen binding fragment thereof; class I, III and IV antibody or antigen binding fragment thereof; class II, III and IV antibody or antigen binding fragment thereof; or class I, II, III and IV antibody or antigen binding fragment thereof. In some aspects, the anti-SARS-CoV-2 virus antibody or an antigen binding fragment thereof is bispecific, trispecific or tetraspecific and has greater neutralization potency against SARS-CoV-2 virus than a monospecific anti-SARS-CoV-2 virus antibody or an antigen binding fragment thereof that specifically binds to one of the same antigens as the bispecific, trispecific or tetraspecific anti-SARS-CoV-2 virus antibody or an antigen binding fragment thereof. In some aspects, the anti-SARS-CoV-2 virus antibody or an antigen binding fragment thereof is bispecific, trispecific or tetraspecific and has greater neutralization potency against SARS-CoV-2 virus than a mixture of monospecific anti-SARS-CoV-2 virus antibodies or an antigen binding fragments thereof that specifically binds to one of the same antigens as the bispecific, trispecific or tetraspecific anti-SARS-CoV-2 virus antibody or an antigen binding fragment thereof.

In some aspects, the SARS-CoV-2 virus is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an Iota variant (e.g., B.1.526), a Lambda variant (e.g., C.37), an Epsilon variant (e.g., B.1.429. B.1.427 or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1. BA.2.86, EG.5, JN.1, JD.1, EG.5.1, FL.1.5.1, HK.3, HV.1, JD.1.1, JF.1, XBB.1, XBB.1.16. XBB.1.16.6, XBB.2.3.2, JN.1.13.1, KP.2, JN.1.16, JN.1.16.1, JN.1.13, JN.1.18, KP.2.3, LB.1, KP.3, or KP.3.1.1), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof. In some aspects, the Omicron variant is B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1. BA.2.86, EG.5, JN.1, JD.1, EG.5.1, FL.1.5.1, HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, KP.3.1.1, or a combination or variant thereof.

In some aspects, the anti-SARS-CoV-2 virus antibody or an antigen binding fragment thereof comprises one or more variable heavy chain regions (VH) and wherein at least one of the one or more VH has a mutation at an N-glycosylation site. In some aspects, the mutation is at amino acid N62 of the VH region or equivalent thereof. In some aspects, the N62 mutation is N62Q or N62S, or equivalent thereof.

In some aspects, the anti-SARS-CoV-2 virus antibody or an antigen binding fragment thereof comprises at least one variable heavy chain region (VH) and/or at least one variable light chain region (VL). In some aspects, the (a) the at least one VL comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, NNN, SYN, WAS, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 764, 766, 768, 771, 779, 786, 792, 795, 808, 815, 821, 830, 837, 843, 850, 856, 859, 866, 872, 879, 885, and 1066; and/or (b) the at least one VH comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (a) the at least one VL comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 772, 780, 787, 796, 802, 809, 816, 822, 831, 838, 844, 851, 860, 867, 873, 880, and 988; and/or (b) the at least one VH comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 775, 777, 784, 791, 800, 806, 813, 820, 826, 835, 842, 848, 855, 864, 871, 877, and 884.

In some aspects, the anti-SARS-CoV-2 virus antibody or an antigen binding fragment thereof is encoded by a nucleotide sequence at least 65%, at least 70%, at least 75%, at least 80%, at least 85%, at least 90%, at least 95%, at least 99%, or 100% identical to any one of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044.

In some aspects, the anti-SARS-CoV-2 virus antibody or an antigen binding fragment thereof comprises an amino acid sequence at least 65%, at least 70%, at least 75%, at least 80%, at least 85%, at least 90%, at least 95%, at least 99%, or 100% identical to any one of SEQ ID NOs: 381-502, 632-633, 720-721, 724-725, 728-729, 732-733, 736-737, 740-741, 744-745, 748-749, 752-753, 760-761, 887-926, 972-977, 984-985, 989-990, 993-994, 997-998, 1001-1002, 1005-1006, 1009-1010, 1013-1014, 1017-1018, 1021-1022, 1025-1026, 1029-1030, 1033-1034, 1037-1038, and 1041-1042.

In some aspects, provided herein are antigen binding polypeptide complexes comprising a first polypeptide and a second polypeptide, wherein the first polypeptide has a structure represented by: VL1-L1-VL2-L2-VH2-L3-VH1-L4-Fc; VL1-L1-VH2-L2-VL2-L3-VH1-L4-Fc; VH1-L1-VH2-L2-VL2-L3-VL1-L4-Fc; or VH1-L1-VL2-L2-VH2-L3-VL1-L4-Fc; wherein the second polypeptide has a structure represented by: VL3-L5-VL4-L6-VH4-L7-VH3-L8-Fc; VL3-L5-VH4-L6-VL4-L7-VH3-L8-Fc; VH3-L5-VH4-L6-VL4-L7-VL3-L8-Fc; or VH3-L5-VL4-L6-VH4-L7-VL3-L8-Fc; wherein: VL1 is a first immunoglobulin light chain variable region that specifically binds to a severe acute respiratory syndrome coronavirus 2 (SARS-CoV-2) protein and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; VL2 is a second immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; VL3 is a third immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; VL4 is a fourth immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOS: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; VH1 is a first immunoglobulin heavy chain variable region that specifically binds to a SARS-CoV-2 protein and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; VH2 is a second immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; VH3 is a third immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; VH4 is a fourth immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; Fc is a region comprising an immunoglobulin heavy chain constant region 2 (CH2), an immunoglobulin heavy chain constant region 3 (CH3), and optionally, an immunoglobulin hinge; and L1-L8 are optional amino acid linkers.

In some aspects, provided herein are antigen binding polypeptide complexes comprising a first polypeptide and a second polypeptide; wherein the first polypeptide has a structure represented by: VL1-L1-VL2-L2-VH2-L3-VH1-L4-Fc; VL1-L1-VH2-L2-VL2-L3-VH1-L4-Fc; VH1-L1-VH2-L2-VL2-37-VL1-L4-Fc; or VH1-L1-VL2-L2-VH2-L3-VL1-L4-Fc; wherein the second polypeptide has a structure represented by: VL3-L5-VL4-L6-VH4-L7-VH3-L8-Fc; VL3-L5-VH4-L6-VL4-L7-VH3-L8-Fc; VH3-L5-VH4-L6-VL4-L7-VL3-L8-Fc; or VH3-L5-VL4-L6-VH4-L7-VL3-L8-Fc; wherein: VL1 is a first immunoglobulin light chain variable region that specifically binds to a severe acute respiratory syndrome coronavirus 2 (SARS-CoV-2) protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL2 is a second immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL3 is a third immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL4 is a fourth immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VH1 is a first immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH2 is a second immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH3 is a third immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH4 is a fourth immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; Fc is a region comprising an immunoglobulin heavy chain constant region 2 (CH2), an immunoglobulin heavy chain constant region 3 (CH3), and optionally, an immunoglobulin hinge; and L1-L8 are optional amino acid linkers.

In some aspects, the antigen binding polypeptide complexes comprise a first polypeptide and a second polypeptide; wherein the first polypeptide has a structure represented by: VL1-L1-VL2-L2-VH2-L3-VH1-L4-Fc; or VH1-L1-VH2-L2-VL2-L3-VL1-L4-Fc; wherein the second polypeptide has a structure represented by: VL3-L5-VL4-L6-VH4-L7-VH3-L8-Fc; or VH3-L5-VH4-L6-VL4-L7-VL3-L8-Fc; wherein: VL1 is a first immunoglobulin light chain variable region that specifically binds to a severe acute respiratory syndrome coronavirus 2 (SARS-CoV-2) protein and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL1 is a first immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL2 is a second immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL2 is a second light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL3 is a third immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOS: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL3 is a third immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL4 is a fourth immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL4 is a fourth immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VH1 is a first immunoglobulin heavy chain variable region that specifically binds to a SARS-CoV-2 protein and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH1 is a first immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH2 is a second immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH2 is a second immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH3 is a third immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH3 is a third immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH4 is a fourth immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH4 is a fourth immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; Fc is a region comprising an immunoglobulin heavy chain constant region 2 (CH2), an immunoglobulin heavy chain constant region 3 (CH3), and optionally, an immunoglobulin hinge; and L1-L8 are optional amino acid linkers.

In some aspects, provided herein are antigen binding polypeptide complexes comprising a first polypeptide and a second polypeptide; wherein the first polypeptide has a structure represented by: VL1-L1-VL2-L2-VH2-L3-VH1-L4-CH1-L5-CL-L6-Fc; VL1-L1-VH2-L2-VL2-L3-VH1-L4-CH1-L5-CL-L6-Fc; VH1-L1-VH2-L2-VL2-L3-VL1-L4-CH1-L5-CL-L6-Fc; VH1-L1-VL2-L2-VH2-L3-VL1-L4-CH1-L5-CL-L6-Fc; VL1-L1-VL2-L2-VH2-L3-VH1-L4-CL-L5-CH1-L6-Fc; VL1-L1-VH2-L2-VL2-L3-VH1- L4-CL-L5-CH1-L6-Fc; VH1-L1-VH2-L2-VL2-L3-VL1-L4-CL-L5-CH1-L6-Fc; or VH1-L1-VL2-L2-VH2-L3-VL1-L4-CL-L5-CH1-L6-Fc; wherein the second polypeptide has a structure represented by: VL3-L7-VL4-L8-VH4-L9-VH3-L10-CH1-L11-CL-L12-Fc; VL3-L7-VH4-L8-VL4-L9-VH3-L10-CH1-L11-CL-L12- Fc; VH3-L7-VH4-L8-VL4-L9-VL3-L10-CH1-L11-CL-L12-Fc; VH3-L7-VL4-L8-VH4-L9-VL3-L10-CH1-L11-CL-L12-Fc; VL3-L7-VL4-L8-VH4-L9-VH3-L10-CL-L11-CH1-L12-Fc; VL3-L7-VH4-L8-VL4-L9-VH3-L10-CL-L11-CH1-L12-Fc; VH3-L7-VH4-L8-VL4-L9-VL3-L10-CL-L11-CH1-L12-Fc; or VH3-L7-VL4-L8-VH4-L9-VL3-L10-CL-L11-CH1-L12-Fc; wherein: VL1 is a first immunoglobulin light chain variable region that specifically binds to a severe acute respiratory syndrome coronavirus 2 (SARS-CoV-2) protein and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL1 is a first immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880); VL2 is a second immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL2 is a second light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL3 is a third immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL3 is a third immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL4 is a fourth immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL4 is a fourth immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VH1 is a first immunoglobulin heavy chain variable region that specifically binds to a SARS-CoV-2 protein and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH1 is a first immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH2 is a second immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH2 is a second immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH3 is a third immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOS: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH3 is a third immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH4 is a fourth immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH4 is a fourth immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L12 are optional amino acid linkers.

In some aspects, provided herein are antigen binding polypeptide complexes comprising two antigen binding polypeptides wherein at least one antigen binding polypeptide has a structure represented by: VL1-L1-VL2-L2-VH2-L3-VH1-L4-Fc-L5-Fc; VL1-L1-VH2-L2-VL2-L3-VH1-L4-Fc-L5-Fc; VH1-L1-VH2-L2-VL2-L3-VL1-L4-Fc-L5-Fc; or VH1-L1-VL2-L2-VH2-L3-VL1-L4-Fc-L5-Fc; wherein: VL1 is a first immunoglobulin light chain variable region that specifically binds to a severe acute respiratory syndrome coronavirus 2 (SARS-CoV-2) protein and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL1 is a first immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL2 is a second immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOS: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL2 is a second immunoglobulin light chain variable region that specifically binds a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VH1 is a first immunoglobulin heavy chain variable region that specifically binds to a SARS-CoV-2 protein and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH1 is a first immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH2 is a second immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH2 is a second immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; Fc is a region comprising an immunoglobulin heavy chain constant region 2 (CH2), an immunoglobulin heavy chain constant region 3 (CH3), and optionally, an immunoglobulin hinge; and L1-L5 are optional amino acid linkers.

In some aspects, the antigen binding polypeptides or antigen binding polypeptide complexes comprise a polypeptide having a structure represented by: VL1-L1-VL2-L2-VH2-L3-VH1-L4-Fc-L5-Fc; or VH1-L1-VH2-L2-VL2-L3-VL1-L4-Fc-L5-Fc; wherein: VL1 is a first immunoglobulin light chain variable region that specifically binds to a severe acute respiratory syndrome coronavirus 2 (SARS-CoV-2) protein and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL1 is a first immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL2 is a second immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL2 is a second immunoglobulin light chain variable region that specifically binds a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VH1 is a first immunoglobulin heavy chain variable region that specifically binds to a SARS-CoV-2 protein and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH1 is a first immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH2 is a second immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH2 is a second immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; Fc is a region comprising an immunoglobulin heavy chain constant region 2 (CH2), an immunoglobulin heavy chain constant region 3 (CH3), and optionally, an immunoglobulin hinge; and L1-L5 are optional amino acid linkers.

In some aspects, any one of the optional linkers comprised in the antigen binding polypeptide complexes disclosed herein each independently have a length of from 0 amino acids to about 50 amino acids. In some aspects, any one of the optional linkers not having a length of 0 amino acids each independently comprise an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptide complexes disclosed herein further comprise an Fc region, wherein the Fc region comprises at least one knob-into-hole modification or Fc effector function knockout mutation. In some aspects, the antigen binding polypeptide complexes disclosed herein are IgG1 or IgG4 antibodies, or antigen binding fragments thereof, and the knob-into-hole modification comprises: (i) knob substitutions of S354C and T366W; (ii) hole substitutions of Y349C, T366S, L368A and Y407V; or (iii) a combination thereof; or equivalent thereof, based on the EU numbering scheme. In some aspects, the antigen binding polypeptide complexes disclosed herein are IgG1 or IgG4 antibodies, or an antigen binding fragments thereof, and the Fc effector function knockout mutation is L234A, L235A, P239A, or equivalent thereof, or a combination thereof, based on the EU numbering scheme.

In some aspects, the antigen binding polypeptide complexes disclosed herein specifically bind to a SARS-CoV-2 protein with an equilibrium dissociation constant (K_{D})) of from about 10 μM to about 1 pM. In some aspects, the antigen binding polypeptide complexes disclosed herein are bispecific, trispecific or tetraspecific and have greater neutralization potency against SARS-CoV-2 virus than a monospecific antigen binding polypeptide complex that specifically binds to one of the same antigens as the bispecific, trispecific or tetraspecific antigen binding polypeptide complex.

In some aspects, the antigen binding polypeptide complexes disclosed herein are bispecific, trispecific or tetraspecific and have greater neutralization potency against SARS-CoV-2 virus than a mixture of monospecific antigen binding polypeptide complexes that specifically bind to the same antigens as the bispecific, trispecific or tetraspecific antigen binding polypeptide complex.

In some aspects, the SARS-CoV-2 virus is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an Iota variant (e.g., B.1.526), a Lambda variant (e.g., C.37), an Epsilon variant (e.g., B.1.429. B.1.427, or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1. BA.2.86, EG.5, JN.1, JD.1, EG.5.1, FL.1.5.1. HK.3, HV.1, JD.1.1, JF.1, XBB.1, XBB.1.16. XBB.1.16.6, or XBB.2.3.2), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof.

In some aspects, provided herein are anti-SARS-CoV-2 antibodies, or antigen binding fragments thereof, comprising one or more antigen binding polypeptides or antigen binding polypeptide complexes disclosed herein, wherein the anti-SARS-CoV-2 antibody or antigen binding fragment thereof exhibits improved prevention of immune escape compared to (i) a monovalent anti-SARS-CoV-2 antibody or an antigen binding fragment thereof that binds to one of the same antigens as the anti-SARS-CoV-2 antibody or antigen binding fragment thereof, and/or (ii) a combination of monovalent anti-SARS-CoV-2 antibodies or antigen binding fragments thereof that binds to the same antigens as the anti-SARS-CoV-2 antibody or antigen binding fragment thereof.

In some aspects, provided herein are pharmaceutical compositions comprising an antigen binding polypeptide complex or an anti-SARS-CoV-2 antibody or antigen binding fragment thereof disclosed herein and a pharmaceutically acceptable carrier.

In some aspects, provided herein are kits comprising an antigen binding polypeptide complex, an anti-SARS-CoV-2 antibody or antigen binding fragment thereof, or a pharmaceutical composition disclosed herein.

In some aspects, provided herein are methods of preventing or treating a SARS-CoV-2 viral infection in a subject in need thereof, comprising administering to the subject a therapeutically effective amount of an antigen binding polypeptide complex, an anti-SARS-CoV-2 antibody or antigen binding fragment thereof, or a pharmaceutical composition disclosed herein.

In some aspects, provided herein are methods of preventing or treating coronavirus disease 2019 (COVID-19) in a subject in need thereof, comprising administering to the subject a therapeutically effective amount of an antigen binding polypeptide complex, an anti-SARS-CoV-2 antibody or antigen binding fragment thereof, or a pharmaceutical composition disclosed herein.

In some aspects, provided herein are methods of diagnosing a subject as being infected with a SARS-CoV-2 virus or suspected of being infected with a SARS-CoV-2 virus, comprising (i) contacting a sample obtained from the subject with an antigen binding polypeptide complex or with an anti-SARS-CoV-2 antibody or antigen binding fragment thereof disclosed herein; (ii) detecting the presence or absence of a virus complex which contains the polypeptide complex or a fragment thereof, or the antibody or a fragment thereof, and a SARS-CoV-2 virus, virion or fragment thereof; and (iii) diagnosing the subject as being infected with a SARS-CoV-2 virus or suspected of being infected with a SARS-CoV-2 virus when the presence of the virus complex is detected.

In some aspects, provided herein are methods of diagnosing a subject as not being infected with a SARS-CoV-2 virus or not suspected of being infected with a SARS-CoV-2 virus, comprising (i) contacting a sample obtained from the subject with an antigen binding polypeptide complex or with an anti-SARS-CoV-2 antibody or antigen binding fragment thereof disclosed herein; (ii) detecting the presence or absence of a virus complex which contains the polypeptide complex or a fragment thereof, or the antibody or a fragment thereof and a SARS-CoV-2 virus, virion or fragment thereof; and (iii) diagnosing the subject as not being infected with a SARS-CoV-2 virus or not suspected of being infected with a SARS-CoV-2 virus when the presence of the virus complex is not detected.

In some aspects, provided herein are methods of preventing immune escape of a SARS-CoV-2 virus in a subject infected with the SARS-CoV-2 virus, comprising administering to the subject an effective amount of an antigen binding polypeptide complex or anti-SARS-CoV-2 antibody or antigen binding fragment thereof disclosed herein that exhibits improved prevention of immune escape compared to (i) a monovalent anti-SARS-CoV-2 antibody or antigen binding fragment thereof that binds to one of the same antigens as the polypeptide complex or anti-SARS-CoV-2 antibody or antigen binding fragment thereof, and/or (ii) a combination of monovalent anti-SARS-CoV-2 antibodies or antigen binding fragments thereof that binds to the same antigens as the polypeptide complex or anti-SARS-CoV-2 antibody or antigen binding fragment thereof.

In some aspects, provided herein are methods of increasing neutralization potency against a SARS-CoV-2 virus in a subject infected with the SARS-CoV-2 virus, comprising administering to the subject an effective amount of an antigen binding polypeptide complex or anti-SARS-CoV-2 antibody or antigen binding fragment thereof disclosed herein that exhibits increased neutralization potency compared to (i) a monovalent anti-SARS-CoV-2 antibody or antigen binding fragment thereof that binds to one of the same antigens as the polypeptide complex or anti-SARS-CoV-2 antibody or antigen binding fragment thereof and/or (ii) a combination of monovalent anti-SARS-CoV-2 antibodies or antigen binding fragments thereof that binds to the same antigens as the polypeptide complex or anti-SARS-CoV-2 antibody or antigen binding fragment thereof.

In some aspects, provided herein are an antigen binding polypeptide complex or antibody or antigen binding fragment thereof comprising: (a) an antigen binding polypeptide having a structure represented by: VL1-L1-VH1 or VH1-L1-VL1; wherein: VL1 is a first immunoglobulin light chain variable region that specifically binds to a severe acute respiratory syndrome coronavirus 2 (SARS-CoV-2) protein; VH1 is a first immunoglobulin heavy chain variable region that specifically binds to a SARS-CoV-2 protein; and L1 and L2 are optional amino acid linkers; or an antigen binding polypeptide having a structure represented by: VL1-L1-VL2-L2-VH2-L3-VH1; VL1-L1-VH2-L2-VL2-L3-VH1; VH1-L1-VH2-L2-VL2-L3-VL1; or VH1-L1-VL2-L2-VH2-L3-VL1; wherein: VL1 is a first immunoglobulin light chain variable region that specifically binds to a SARS-CoV-2 protein and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL1 is a first immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL2 is a second immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL2 is a second immunoglobulin light chain variable region that specifically binds a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VH1 is a first immunoglobulin heavy chain variable region that specifically binds to a SARS-CoV-2 protein and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH1 is a first immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH2 is a second immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH2 is a second immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; and L1-L3 are optional amino acid linkers; and wherein (a) is selected from any one of the more specific aspects provided herein for an antigen binding polypeptide in an antigen binding polypeptide complex having no more than three polypeptide chains; (b) an antigen binding polypeptide having a structure represented by: VL1-L1-VH1 or VH1-L1-VL1; wherein: VL1 is a first immunoglobulin light chain variable region that specifically binds to a SARS-CoV-2 protein and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL1 is a first immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VH1 is a first immunoglobulin heavy chain variable region that specifically binds to a SARS-CoV-2 protein and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH1 is a first immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; and L1 and L2 are optional amino acid linkers; or an antigen binding polypeptide having a structure represented by: VL1-L1-VL2-L2-VH2-L3-VH1; VL1-L1-VH2-L2-VL2-L3-VH1; VH1-L1-VH2-L2-VL2-L3-VL1; or VH1-L1-VL2-L2-VH2-L3-VL1; wherein: VL1 is a first immunoglobulin light chain variable region that specifically binds to a SARS-CoV-2 protein and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL1 is a first immunoglobulin light chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VL2 is a second immunoglobulin light chain variable region that specifically binds to a SARS-CoV-2 protein and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and a LCDR3 comprising an amino acid sequence at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066, or VL2 is a second immunoglobulin light chain variable region that specifically binds a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; VH1 is a first immunoglobulin heavy chain variable region that specifically binds to a SARS-CoV-2 protein and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH1 is a first immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; VH2 is a second immunoglobulin heavy chain variable region that specifically binds to a SARS-CoV-2 protein and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; a HCDR2 comprising an amino acid sequence at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and a HCDR3 comprising an amino acid sequence at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069, or VH2 is a second immunoglobulin heavy chain variable region that specifically binds to a SARS-CoV-2 protein comprising an amino acid sequence at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; and L1-L3 are optional amino acid linkers; wherein (b) is selected from any one of the more specific aspects provided herein for an antigen binding polypeptide in an antigen binding polypeptide complex having no more than three polypeptide chains; and wherein the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide, antigen binding polypeptide complex, antibody or antigen binding fragment thereof, polypeptide, polynucleotide, vector, host cell, mRNA, LNP, CAR, immune cell, pharmaceutical composition, kit, or method disclosed herein comprises or encodes:
a VL at least 65%, at least 70%, at least 75%, at least 80%, at least 85%, at least 90%, at least 95%, at least 99%, or 100% identical to SEQ ID NO: 4 and a VH at least 65%, at least 70%, at least 75%, at least 80%, at least 85%, at least 90%, at least 95%, at least 99%, or 100% identical to SEQ ID NO: 8;
a VL at least 65%, at least 70%, at least 75%, at least 80%, at least 85%, at least 90%, at least 95%, at least 99%, or 100% identical to SEQ ID NO: 11 and a VH at least 65%, at least 70%, at least 75%, at least 80%, at least 85%, at least 90%, at least 95%, at least 99%, or 100% identical to SEQ ID NO: 14;
a VL at least 65%, at least 70%, at least 75%, at least 80%, at least 85%, at least 90%, at least 95%, at least 99%, or 100% identical to SEQ ID NO: 17 and a VH at least 65%, at least 70%, at least 75%, at least 80%, at least 85%, at least 90%, at least 95%, at least 99%, or 100% identical to SEQ ID NO: 21;
a VL at least 65%, at least 70%, at least 75%, at least 80%, at least 85%, at least 90%, at least 95%, at least 99%, or 100% identical to SEQ ID NO: 17 and a VH at least 65%, at least 70%, at least 75%, at least 80%, at least 85%, at least 90%, at least 95%, at least 99%, or 100% identical to SEQ ID NO: 22;
a VL at least 65%, at least 70%, at least 75%, at least 80%, at least 85%, at least 90%, at least 95%, at least 99%, or 100% identical to SEQ ID NO: 25 and a VH at least 65%, at least 70%, at least 75%, at least 80%, at least 85%, at least 90%, at least 95%, at least 99%, or 100% identical to SEQ ID NO: 29;
a VL at least 65%, at least 70%, at least 75%, at least 80%, at least 85%, at least 90%, at least 95%, at least 99%, or 100% identical to SEQ ID NO: 33 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 39 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 43 and a VH at least 65%, at least 70%, at least 75%, at least 80%, at least 85%, at least 90%, at least 95%, at least 99%, or 100% identical to SEQ ID NO: 47;
a VL at least 65%, at least 70%, at least 75%, at least 80%, at least 85%, at least 90%, at least 95%, at least 99%, or 100% identical to SEQ ID NO: 988 and a VH at least 65%, at least 70%, at least 75%, at least 80%, at least 85%, at least 90%, at least 95%, at least 99%, or 100% identical to SEQ ID NO: 57;
a VL at least 65%, at least 70%, at least 75%, at least 80%, at least 85%, at least 90%, at least 95%, at least 99%, or 100% identical to SEQ ID NO: 50 and a VH at least 65%, at least 70%, at least 75%, at least 80%, at least 85%, at least 90%, at least 95%, at least 99%, or 100% identical to SEQ ID NO: 54;
a VL at least 65%, at least 70%, at least 75%, at least 80%, at least 85%, at least 90%, at least 95%, at least 99%, or 100% identical to SEQ ID NO: 60 and a VH at least 65%, at least 70%, at least 75%, at least 80%, at least 85%, at least 90%, at least 95%, at least 99%, or 100% identical to SEQ ID NO: 64;
a VL at least 65%, at least 70%, at least 75%, at least 80%, at least 85%, at least 90%, at least 95%, at least 99%, or 100% identical to SEQ ID NO: 67 and a VH at least 65%, at least 70%, at least 75%, at least 80%, at least 85%, at least 90%, at least 95%, at least 99%, or 100% identical to SEQ ID NO: 71;
a VL at least 65%, at least 70%, at least 75%, at least 80%, at least 85%, at least 90%, at least 95%, at least 99%, or 100% identical to SEQ ID NO: 67 and a VH at least 65%, at least 70%, at least 75%, at least 80%, at least 85%, at least 90%, at least 95%, at least 99%, or 100% identical to SEQ ID NO: 626;
a VL at least 65%, at least 70%, at least 75%, at least 80%, at least 85%, at least 90%, at least 95%, at least 99%, or 100% identical to SEQ ID NO: 74 and a VH at least 65%, at least 70%, at least 75%, at least 80%, at least 85%, at least 90%, at least 95%, at least 99%, or 100% identical to SEQ ID NO: 78;
a VL at least 65%, at least 70%, at least 75%, at least 80%, at least 85%, at least 90%, at least 95%, at least 99%, or 100% identical to SEQ ID NO: 81 and a VH at least 65%, at least 70%, at least 75%, at least 80%, at least 85%, at least 90%, at least 95%, at least 99%, or 100% identical to SEQ ID NO: 85;
a VL at least 65%, at least 70%, at least 75%, at least 80%, at least 85%, at least 90%, at least 95%, at least 99%, or 100% identical to SEQ ID NO: 88 and a VH at least 65%, at least 70%, at least 75%, at least 80%, at least 85%, at least 90%, at least 95%, at least 99%, or 100% identical to SEQ ID NO: 92;
a VL at least 65%, at least 70%, at least 75%, at least 80%, at least 85%, at least 90%, at least 95%, at least 99%, or 100% identical to SEQ ID NO: 95 and a VH at least 65%, at least 70%, at least 75%, at least 80%, at least 85%, at least 90%, at least 95%, at least 99%, or 100% identical to SEQ ID NO: 98;
a VL at least 65%, at least 70%, at least 75%, at least 80%, at least 85%, at least 90%, at least 95%, at least 99%, or 100% identical to SEQ ID NO: 101 and a VH at least 65%, at least 70%, at least 75%, at least 80%, at least 85%, at least 90%, at least 95%, at least 99%, or 100% identical to SEQ ID NO: 104;
a VL at least 65%, at least 70%, at least 75%, at least 80%, at least 85%, at least 90%, at least 95%, at least 99%, or 100% identical to SEQ ID NO: 107 and a VH at least 65%, at least 70%, at least 75%, at least 80%, at least 85%, at least 90%, at least 95%, at least 99%, or 100% identical to SEQ ID NO: 111;
a VL at least 65%, at least 70%, at least 75%, at least 80%, at least 85%, at least 90%, at least 95%, at least 99%, or 100% identical to SEQ ID NO: 114 and a VH at least 65%, at least 70%, at least 75%, at least 80%, at least 85%, at least 90%, at least 95%, at least 99%, or 100% identical to SEQ ID NO: 118;
a VL at least 65%, at least 70%, at least 75%, at least 80%, at least 85%, at least 90%, at least 95%, at least 99%, or 100% identical to SEQ ID NO: 121 and a VH at least 65%, at least 70%, at least 75%, at least 80%, at least 85%, at least 90%, at least 95%, at least 99%, or 100% identical to SEQ ID NO: 125;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 137;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 132 and a VH at least 65%, at least 70%, at least 75%, at least 80%, at least 85%, at least 90%, at least 95%, at least 99%, or 100% identical to SEQ ID NO: 136;
a VL at least 65%, at least 70%, at least 75%, at least 80%, at least 85%, at least 90%, at least 95%, at least 99%, or 100% identical to SEQ ID NO: 140 and a VH at least 65%, at least 70%, at least 75%, at least 80%, at least 85%, at least 90%, at least 95%, at least 99%, or 100% identical to SEQ ID NO: 144;
a VL at least 65%, at least 70%, at least 75%, at least 80%, at least 85%, at least 90%, at least 95%, at least 99%, or 100% identical to SEQ ID NO: 147 and a VH at least 65%, at least 70%, at least 75%, at least 80%, at least 85%, at least 90%, at least 95%, at least 99%, or 100% identical to SEQ ID NO: 151;
a VL at least 65%, at least 70%, at least 75%, at least 80%, at least 85%, at least 90%, at least 95%, at least 99%, or 100% identical to SEQ ID NO: 154 and a VH at least 65%, at least 70%, at least 75%, at least 80%, at least 85%, at least 90%, at least 95%, at least 99%, or 100% identical to SEQ ID NO: 158;
a VL at least 65%, at least 70%, at least 75%, at least 80%, at least 85%, at least 90%, at least 95%, at least 99%, or 100% identical to SEQ ID NO: 161 and a VH at least 65%, at least 70%, at least 75%, at least 80%, at least 85%, at least 90%, at least 95%, at least 99%, or 100% identical to SEQ ID NO: 165;
a VL at least 65%, at least 70%, at least 75%, at least 80%, at least 85%, at least 90%, at least 95%, at least 99%, or 100% identical to SEQ ID NO: 168 and a VH at least 65%, at least 70%, at least 75%, at least 80%, at least 85%, at least 90%, at least 95%, at least 99%, or 100% identical to SEQ ID NO: 171;
a VL at least 65%, at least 70%, at least 75%, at least 80%, at least 85%, at least 90%, at least 95%, at least 99%, or 100% identical to SEQ ID NO: 174 and a VH at least 65%, at least 70%, at least 75%, at least 80%, at least 85%, at least 90%, at least 95%, at least 99%, or 100% identical to SEQ ID NO: 178;
a VL at least 65%, at least 70%, at least 75%, at least 80%, at least 85%, at least 90%, at least 95%, at least 99%, or 100% identical to SEQ ID NO: 181 and a VH at least 65%, at least 70%, at least 75%, at least 80%, at least 85%, at least 90%, at least 95%, at least 99%, or 100% identical to SEQ ID NO: 185;
a VL at least 65%, at least 70%, at least 75%, at least 80%, at least 85%, at least 90%, at least 95%, at least 99%, or 100% identical to SEQ ID NO: 188 and a VH at least 65%, at least 70%, at least 75%, at least 80%, at least 85%, at least 90%, at least 95%, at least 99%, or 100% identical to SEQ ID NO: 191;
a VL at least 65%, at least 70%, at least 75%, at least 80%, at least 85%, at least 90%, at least 95%, at least 99%, or 100% identical to SEQ ID NO: 194 and a VH at least 65%, at least 70%, at least 75%, at least 80%, at least 85%, at least 90%, at least 95%, at least 99%, or 100% identical to SEQ ID NO: 198;
a VL at least 65%, at least 70%, at least 75%, at least 80%, at least 85%, at least 90%, at least 95%, at least 99%, or 100% identical to SEQ ID NO: 201 and a VH at least 65%, at least 70%, at least 75%, at least 80%, at least 85%, at least 90%, at least 95%, at least 99%, or 100% identical to SEQ ID NO: 204;
a VL at least 65%, at least 70%, at least 75%, at least 80%, at least 85%, at least 90%, at least 95%, at least 99%, or 100% identical to SEQ ID NO: 207 and a VH at least 65%, at least 70%, at least 75%, at least 80%, at least 85%, at least 90%, at least 95%, at least 99%, or 100% identical to SEQ ID NO: 211;
a VL at least 65%, at least 70%, at least 75%, at least 80%, at least 85%, at least 90%, at least 95%, at least 99%, or 100% identical to SEQ ID NO: 214 and a VH at least 65%, at least 70%, at least 75%, at least 80%, at least 85%, at least 90%, at least 95%, at least 99%, or 100% identical to SEQ ID NO: 218;
a VL at least 65%, at least 70%, at least 75%, at least 80%, at least 85%, at least 90%, at least 95%, at least 99%, or 100% identical to SEQ ID NO: 221 and a VH at least 65%, at least 70%, at least 75%, at least 80%, at least 85%, at least 90%, at least 95%, at least 99%, or 100% identical to SEQ ID NO: 225;
a VL at least 65%, at least 70%, at least 75%, at least 80%, at least 85%, at least 90%, at least 95%, at least 99%, or 100% identical to SEQ ID NO: 228 and a VH at least 65%, at least 70%, at least 75%, at least 80%, at least 85%, at least 90%, at least 95%, at least 99%, or 100% identical to SEQ ID NO: 231;
a VL at least 65%, at least 70%, at least 75%, at least 80%, at least 85%, at least 90%, at least 95%, at least 99%, or 100% identical to SEQ ID NO: 234 and a VH at least 65%, at least 70%, at least 75%, at least 80%, at least 85%, at least 90%, at least 95%, at least 99%, or 100% identical to SEQ ID NO: 238;
a VL at least 65%, at least 70%, at least 75%, at least 80%, at least 85%, at least 90%, at least 95%, at least 99%, or 100% identical to SEQ ID NO: 240 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 243 and a VH at least 65%, at least 70%, at least 75%, at least 80%, at least 85%, at least 90%, at least 95%, at least 99%, or 100% identical to SEQ ID NO: 245;
a VL at least 65%, at least 70%, at least 75%, at least 80%, at least 85%, at least 90%, at least 95%, at least 99%, or 100% identical to SEQ ID NO: 247 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 250 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 251 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 252 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 33 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 255 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 255 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 255 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 258;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 260;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 262;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 262;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 266;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 268;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 270;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 331;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 272;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 275;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 277;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 279;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 280;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 282;
a VL at least 65%, at least 70%, at least 75%, at least 80%, at least 85%, at least 90%, at least 95%, at least 99%, or 100% identical to SEQ ID NO: 284 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 323 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 286 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 289 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 291 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 293 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 295 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 247 and a VH at least 65%, at least 70%, at least 75%, at least 80%, at least 85%, at least 90%, at least 95%, at least 99%, or 100% identical to SEQ ID NO: 282;
a VL at least 65%, at least 70%, at least 75%, at least 80%, at least 85%, at least 90%, at least 95%, at least 99%, or 100% identical to SEQ ID NO: 250 and a VH at least 65%, at least 70%, at least 75%, at least 80%, at least 85%, at least 90%, at least 95%, at least 99%, or 100% identical to SEQ ID NO: 282;
a VL at least 65%, at least 70%, at least 75%, at least 80%, at least 85%, at least 90%, at least 95%, at least 99%, or 100% identical to SEQ ID NO: 39 and a VH at least 65%, at least 70%, at least 75%, at least 80%, at least 85%, at least 90%, at least 95%, at least 99%, or 100% identical to SEQ ID NO: 298;
a VL at least 65%, at least 70%, at least 75%, at least 80%, at least 85%, at least 90%, at least 95%, at least 99%, or 100% identical to SEQ ID NO: 39 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 299 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 300 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 302 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 303 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 305 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 307 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 308 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 310 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 312 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 314 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 315 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 305 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 251 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 255 and a VH at least 65%, at least 70%, at least 75%, at least 80%, at least 85%, at least 90%, at least 95%, at least 99%, or 100% identical to SEQ ID NO: 282;
a VL at least 65%, at least 70%, at least 75%, at least 80%, at least 85%, at least 90%, at least 95%, at least 99%, or 100% identical to SEQ ID NO: 317 and a VH at least 65%, at least 70%, at least 75%, at least 80%, at least 85%, at least 90%, at least 95%, at least 99%, or 100% identical to SEQ ID NO: 321;
a VL at least 65%, at least 70%, at least 75%, at least 80%, at least 85%, at least 90%, at least 95%, at least 99%, or 100% identical to SEQ ID NO: 772 and a VH at least 65%, at least 70%, at least 75%, at least 80%, at least 85%, at least 90%, at least 95%, at least 99%, or 100% identical to SEQ ID NO: 775;
a VL at least 65%, at least 70%, at least 75%, at least 80%, at least 85%, at least 90%, at least 95%, at least 99%, or 100% identical to SEQ ID NO: 43 and a VH at least 65%, at least 70%, at least 75%, at least 80%, at least 85%, at least 90%, at least 95%, at least 99%, or 100% identical to SEQ ID NO: 777;
a VL at least 65%, at least 70%, at least 75%, at least 80%, at least 85%, at least 90%, at least 95%, at least 99%, or 100% identical to SEQ ID NO: 780 and a VH at least 65%, at least 70%, at least 75%, at least 80%, at least 85%, at least 90%, at least 95%, at least 99%, or 100% identical to SEQ ID NO: 784;
a VL at least 65%, at least 70%, at least 75%, at least 80%, at least 85%, at least 90%, at least 95%, at least 99%, or 100% identical to SEQ ID NO: 787 and a VH at least 65%, at least 70%, at least 75%, at least 80%, at least 85%, at least 90%, at least 95%, at least 99%, or 100% identical to SEQ ID NO: 791;
a VL at least 65%, at least 70%, at least 75%, at least 80%, at least 85%, at least 90%, at least 95%, at least 99%, or 100% identical to SEQ ID NO: 796 and a VH at least 65%, at least 70%, at least 75%, at least 80%, at least 85%, at least 90%, at least 95%, at least 99%, or 100% identical to SEQ ID NO: 800;
a VL at least 65%, at least 70%, at least 75%, at least 80%, at least 85%, at least 90%, at least 95%, at least 99%, or 100% identical to SEQ ID NO: 802 and a VH at least 65%, at least 70%, at least 75%, at least 80%, at least 85%, at least 90%, at least 95%, at least 99%, or 100% identical to SEQ ID NO: 806;
a VL at least 65%, at least 70%, at least 75%, at least 80%, at least 85%, at least 90%, at least 95%, at least 99%, or 100% identical to SEQ ID NO: 809 and a VH at least 65%, at least 70%, at least 75%, at least 80%, at least 85%, at least 90%, at least 95%, at least 99%, or 100% identical to SEQ ID NO: 813;
a VL at least 65%, at least 70%, at least 75%, at least 80%, at least 85%, at least 90%, at least 95%, at least 99%, or 100% identical to SEQ ID NO: 816 and a VH at least 65%, at least 70%, at least 75%, at least 80%, at least 85%, at least 90%, at least 95%, at least 99%, or 100% identical to SEQ ID NO: 820;
a VL at least 65%, at least 70%, at least 75%, at least 80%, at least 85%, at least 90%, at least 95%, at least 99%, or 100% identical to SEQ ID NO: 822 and a VH at least 65%, at least 70%, at least 75%, at least 80%, at least 85%, at least 90%, at least 95%, at least 99%, or 100% identical to SEQ ID NO: 826;
a VL at least 65%, at least 70%, at least 75%, at least 80%, at least 85%, at least 90%, at least 95%, at least 99%, or 100% identical to SEQ ID NO: 831 and a VH at least 65%, at least 70%, at least 75%, at least 80%, at least 85%, at least 90%, at least 95%, at least 99%, or 100% identical to SEQ ID NO: 835;
a VL at least 65%, at least 70%, at least 75%, at least 80%, at least 85%, at least 90%, at least 95%, at least 99%, or 100% identical to SEQ ID NO: 838 and a VH at least 65%, at least 70%, at least 75%, at least 80%, at least 85%, at least 90%, at least 95%, at least 99%, or 100% identical to SEQ ID NO: 842;
a VL at least 65%, at least 70%, at least 75%, at least 80%, at least 85%, at least 90%, at least 95%, at least 99%, or 100% identical to SEQ ID NO: 844 and a VH at least 65%, at least 70%, at least 75%, at least 80%, at least 85%, at least 90%, at least 95%, at least 99%, or 100% identical to SEQ ID NO: 848;
a VL at least 65%, at least 70%, at least 75%, at least 80%, at least 85%, at least 90%, at least 95%, at least 99%, or 100% identical to SEQ ID NO: 851 and a VH at least 65%, at least 70%, at least 75%, at least 80%, at least 85%, at least 90%, at least 95%, at least 99%, or 100% identical to SEQ ID NO: 855;
a VL at least 65%, at least 70%, at least 75%, at least 80%, at least 85%, at least 90%, at least 95%, at least 99%, or 100% identical to SEQ ID NO: 860 and a VH at least 65%, at least 70%, at least 75%, at least 80%, at least 85%, at least 90%, at least 95%, at least 99%, or 100% identical to SEQ ID NO: 864;
a VL at least 65%, at least 70%, at least 75%, at least 80%, at least 85%, at least 90%, at least 95%, at least 99%, or 100% identical to SEQ ID NO: 867 and a VH at least 65%, at least 70%, at least 75%, at least 80%, at least 85%, at least 90%, at least 95%, at least 99%, or 100% identical to SEQ ID NO: 871;
a VL at least 65%, at least 70%, at least 75%, at least 80%, at least 85%, at least 90%, at least 95%, at least 99%, or 100% identical to SEQ ID NO: 873 and a VH at least 65%, at least 70%, at least 75%, at least 80%, at least 85%, at least 90%, at least 95%, at least 99%, or 100% identical to SEQ ID NO: 877; or
a VL at least 65%, at least 70%, at least 75%, at least 80%, at least 85%, at least 90%, at least 95%, at least 99%, or 100% identical to SEQ ID NO: 880 and a VH at least 65%, at least 70%, at least 75%, at least 80%, at least 85%, at least 90%, at least 95%, at least 99%, or 100% identical to SEQ ID NO: 884.

In some aspects, in any one of the antigen binding polypeptides or antigen binding polypeptide complexes disclosed herein:
(i) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049;
and/or
(ii) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and/or
(iii) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064.

In some of those aspects, in any one of the antigen binding polypeptides or antigen binding polypeptide complexes disclosed herein:
(i) any one of the VL comprises an amino acid sequence that is at least 65%, at least 70%, at least 75%, at least 80%, at least 85%, at least 90%, at least 95%, at least 99%, or 100% identical to SEQ ID NO: 154; and any one of the VH comprises an amino acid sequence that is at least 65%, at least 70%, at least 75%, at least 80%, at least 85%, at least 90%, at least 95%, at least 99%, or 100% identical to SEQ ID NO: 158; and/or
(ii) any one of the VL comprises an amino acid sequence that is at least 65%, at least 70%, at least 75%, at least 80%, at least 85%, at least 90%, at least 95%, at least 99%, or 100% identical to SEQ ID NO: 147; and any one of the VH comprises an amino acid sequence that is at least 65%, at least 70%, at least 75%, at least 80%, at least 85%, at least 90%, at least 95%, at least 99%, or 100% identical to SEQ ID NO: 151; and/or
(iii) any one of the VL comprises an amino acid sequence that is at least 65%, at least 70%, at least 75%, at least 80%, at least 85%, at least 90%, at least 95%, at least 99%, or 100% identical to SEQ ID NO: 174; and any one of the VH comprises an amino acid sequence that is at least 65%, at least 70%, at least 75%, at least 80%, at least 85%, at least 90%, at least 95%, at least 99%, or 100% identical to SEQ ID NO: 178.

In some aspects, also provided herein are antigen binding polypeptides having a structure, from amino-terminus to carboxy-terminus, represented by:
VL1-L1-VL2-L2-VH2-L3-VH1-L4-CL-L5-CH1;
VL1-L1-VL2-L2-VH2-L3-VH1-L4-CH1-L5-CL;
VL1-L1-VH2-L2-VL2-L3-VH1-L4-CL-L5-CH1;
VL1-L1-VH2-L2-VL2-L3-VH1-L4-CH1-L5-CL;
VH1-L1-VL2-L2-VH2-L3-VL1-L4-CL-L5-CH1;
VH1-L1-VL2-L2-VH2-L3-VL1-L4-CH1-L5-CL;
VH1-L1-VH2-L2-VL2-L3-VL1-L4-CL-L5-CH1;
VH1-L1-VH2-L2-VL2-L3-VL1-L4-CH1-L5-CL;
VL2-L1-VL1-L2-VH1-L3-VH2-L4-CL-L5-CH1;
VL2-L1-VL1-L2-VH1-L3-VH2-L4-CH1-L5-CL;
VL2-L1-VH1-L2-VL1-L3-VH2-L4-CL-L5-CH1;
VL2-L1-VH1-L2-VL1-L3-VH2-L4-CH1-L5-CL;
VH2-L1-VL1-L2-VH1-L3-VL2-L4-CL-L5-CH1;
VH2-L1-VL1-L2-VH1-L3-VL2-L4-CH1-L5-CL;
VH2-L1-VH1-L2-VL1-L3-VL2-L4-CL-L5-CH1; or
VH2-L1-VH1-L2-VL1-L3-VL2-L4-CH1-L5-CL;
wherein:
(i) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049;
and/or
(ii) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and/or
(iii) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064; and
wherein:
CL is an immunoglobulin light chain constant region;
CH1 is an immunoglobulin heavy chain constant region 1; and
L1-L5 are optional amino acid linkers.

In some of those aspects, in those antigen binding polypeptides:
(i) any one of the VL comprises an amino acid sequence that is at least 65%, at least 70%, at least 75%, at least 80%, at least 85%, at least 90%, at least 95%, at least 99%, or 100% identical to SEQ ID NO: 154; and any one of the VH comprises an amino acid sequence that is at least 65%, at least 70%, at least 75%, at least 80%, at least 85%, at least 90%, at least 95%, at least 99%, or 100% identical to SEQ ID NO: 158; and/or
(ii) any one of the VL comprises an amino acid sequence that is at least 65%, at least 70%, at least 75%, at least 80%, at least 85%, at least 90%, at least 95%, at least 99%, or 100% identical to SEQ ID NO: 147; and any one of the VH comprises an amino acid sequence that is at least 65%, at least 70%, at least 75%, at least 80%, at least 85%, at least 90%, at least 95%, at least 99%, or 100% identical to SEQ ID NO: 151; and/or
(iii) any one of the VL comprises an amino acid sequence that is at least 65%, at least 70%, at least 75%, at least 80%, at least 85%, at least 90%, at least 95%, at least 99%, or 100% identical to SEQ ID NO: 174; and any one of the VH comprises an amino acid sequence that is at least 65%, at least 70%, at least 75%, at least 80%, at least 85%, at least 90%, at least 95%, at least 99%, or 100% identical to SEQ ID NO: 178.

In some aspects, also provided herein are antigen binding polypeptides complexes comprising a first polypeptide and a second polypeptide, wherein the first polypeptide has a structure, from amino-terminus to carboxy-terminus, represented by:
VL1-L1-VL2-L2-VH2-L3-VH1-L4-CL-L5-CH1;
VL1-L1-VL2-L2-VH2-L3-VH1-L4-CH1-L5-CL;
VL1-L1-VH2-L2-VL2-L3-VH1-L4-CL-L5-CH1;
VL1-L1-VH2-L2-VL2-L3-VH1-L4-CH1-L5-CL;
VH1-L1-VL2-L2-VH2-L3-VL1-L4-CL-L5-CH1;
VH1-L1-VL2-L2-VH2-L3-VL1-L4-CH1-L5-CL;
VH1-L1-VH2-L2-VL2-L3-VL1-L4-CL-L5-CH1;
VH1-L1-VH2-L2-VL2-L3-VL1-L4-CH1-L5-CL;
VL2-L1-VL1-L2-VH1-L3-VH2-L4-CL-L5-CH1;
VL2-L1-VL1-L2-VH1-L3-VH2-L4-CH1-L5-CL;
VL2-L1-VH1-L2-VL1-L3-VH2-L4-CL-L5-CH1;
VL2-L1-VH1-L2-VL1-L3-VH2-L4-CH1-L5-CL;
VH2-L1-VL1-L2-VH1-L3-VL2-L4-CL-L5-CH1;
VH2-L1-VL1-L2-VH1-L3-VL2-L4-CH1-L5-CL;
VH2-L1-VH1-L2-VL1-L3-VL2-L4-CL-L5-CH1; or
VH2-L1-VH1-L2-VL1-L3-VL2-L4-CH1-L5-CL;
   wherein the second polypeptide has a structure, from amino-terminus to carboxy-terminus, represented by:
VL3-L6-VL4-L7-VH4-L8-VH3-L9-CL-L10-CH1;
VL3-L6-VL4-L7-VH4-L8-VH3-L9-CH1-L10-CL;
VL3-L6-VH4-L7-VL4-L8-VH3-L9-CL-L10-CH1;
VL3-L6-VH4-L7-VL4-L8-VH3-L9-CH1-L10-CL;
VH3-L6-VL4-L7-VH4-L8-VL3-L9-CL-L10-CH1;
VH3-L6-VL4-L7-VH4-L8-VL3-L9-CH1-L10-CL;
VH3-L6-VH4-L7-VL4-L8-VL3-L9-CL-L10-CH1;
VH3-L6-VH4-L7-VL4-L8-VL3-L9-CH1-L10-CL;
VL4-L6-VL3-L7-VH3-L8-VH4-L9-CL-L10-CH1;
VL4-L6-VL3-L7-VH3-L8-VH4-L9-CH1-L10-CL;
VL4-L6-VH3-L7-VL3-L8-VH4-L9-CL-L10-CH1;
VL4-L6-VH3-L7-VL3-L8-VH4-L9-CH1-L10-CL;
VH4-L6-VL3-L7-VH3-L8-VL4-L9-CL-L10-CH1;
VH4-L6-VL3-L7-VH3-L8-VL4-L9-CH1-L10-CL;
VH4-L6-VH3-L7-VL3-L8-VL4-L9-CL-L10-CH1; or
VH4-L6-VH3-L7-VL3-L8-VL4-L9-CH1-L10-CL; and
wherein:
(i) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049;
and/or
(ii) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and/or
(iii) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064; and
wherein:
CL is an immunoglobulin light chain constant region;
CH1 is an immunoglobulin heavy chain constant region 1; and
L1-L10 are optional amino acid linkers.

In some of those aspects, in those antigen binding polypeptides complexes:
(i) any one of the VL comprises an amino acid sequence that is at least 65%, at least 70%, at least 75%, at least 80%, at least 85%, at least 90%, at least 95%, at least 99%, or 100% identical to SEQ ID NO: 154; and any one of the VH comprises an amino acid sequence that is at least 65%, at least 70%, at least 75%, at least 80%, at least 85%, at least 90%, at least 95%, at least 99%, or 100% identical to SEQ ID NO: 158; and/or
(ii) any one of the VL comprises an amino acid sequence that is at least 65%, at least 70%, at least 75%, at least 80%, at least 85%, at least 90%, at least 95%, at least 99%, or 100% identical to SEQ ID NO: 147; and any one of the VH comprises an amino acid sequence that is at least 65%, at least 70%, at least 75%, at least 80%, at least 85%, at least 90%, at least 95%, at least 99%, or 100% identical to SEQ ID NO: 151; and/or
(iii) any one of the VL comprises an amino acid sequence that is at least 65%, at least 70%, at least 75%, at least 80%, at least 85%, at least 90%, at least 95%, at least 99%, or 100% identical to SEQ ID NO: 174; and any one of the VH comprises an amino acid sequence that is at least 65%, at least 70%, at least 75%, at least 80%, at least 85%, at least 90%, at least 95%, at least 99%, or 100% identical to SEQ ID NO: 178.

In some of the aspects above, the antigen binding polypeptides or one or both of the antigen binding polypeptides comprised in the antigen binding polypeptide complexes disclosed herein further comprises an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380. In some aspects, the Fc region comprises one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise amino acid substitutions:
(i) L234A and L235A (LA);
(ii) M428L and N434S (LS);
(iii) L234F and L235E;
(iv) L234F, L235E, and P331S (TM); and/or
(v) M252Y, S254T, and T256E (YTE);
or equivalent thereof, based on the EU numbering scheme.

In some aspects, the Fc region comprises at least one knob-into-hole modification or Fc effector function knockout mutation. In some aspects, the antigen binding polypeptide or the antigen binding polypeptide complex is an IgG1 or IgG4 antibody or antigen binding fragment thereof and the knob-into-hole modification comprises:
(i) knob substitutions of S354C and T366W;
(ii) hole substitutions of Y349C, T366S, L368A and Y407V;
(iii) a combination thereof;
or equivalent thereof, based on the EU numbering scheme.

In some aspects, the antigen binding polypeptide or the antigen binding polypeptide complex is an IgG1 or IgG4 antibody or antigen binding fragment thereof and the Fc effector function knockout mutation is L234A, L235A, P239A, or equivalent thereof, or a combination thereof, based on the EU numbering scheme.

In some aspects, any one of the optional linkers each independently have a length of from 0 amino acids to about 50 amino acids. In some aspects, any one of the optional linkers each independently have a length of from about 5 amino acids to about 40 amino acids. In some aspects, one or more of the optional linkers each independently are non-immunogenic. In some aspects, one or more of the optional linkers each independently do not contain a consensus T cell epitope. In some aspects, one or more of the optional linkers each independently comprises an amino acid sequence of G or A; or an amino acid sequence at least 65%, at least 70%, at least 75%, at least 80%, at least 85%, at least 90%, at least 95%, at least 99%, or 100% identical to an amino acid sequence of GSS or ASG; or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, one or more of the optional linkers each independently comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, any one of the antigen binding polypeptides, antigen binding polypeptide complexes, antibodies or antigen binding fragments thereof, polypeptides, or anti-SARS-CoV-2 virus antibodies or an antigen binding fragments thereof comprise one or more CL comprising an amino acid sequence that is at least 65%, at least 70%, at least 75%, at least 80%, at least 85%, at least 90%, at least 95%, at least 99%, or 100% identical to any one of SEQ ID NOs: 374, 637, or 1092-1095.

In some aspects, any one of the antigen binding polypeptides, antigen binding polypeptide complexes, antibodies or antigen binding fragments thereof, polypeptides, or anti-SARS-CoV-2 virus antibodies or an antigen binding fragments thereof comprise one or more CH1 comprising an amino acid sequence that is at least 65%, at least 70%, at least 75%, at least 80%, at least 85%, at least 90%, at least 95%, at least 99%, or 100% identical to any one of SEQ ID NOs: 375, 634, 1070, 1071, or 1086-1091. In some aspects, any one of the antigen binding polypeptides, antigen binding polypeptide complexes, antibodies or antigen binding fragments thereof, polypeptides, or anti-SARS-CoV-2 virus antibodies or an antigen binding fragments thereof comprise one or more CH2 comprising an amino acid sequence that is at least 65%, at least 70%, at least 75%, at least 80%, at least 85%, at least 90%, at least 95%, at least 99%, or 100% identical to any one of SEQ ID NOs: 1075-1078.

In some aspects, any one of the antigen binding polypeptides, antigen binding polypeptide complexes, antibodies or antigen binding fragments thereof, polypeptides, or anti-SARS-CoV-2 virus antibodies or an antigen binding fragments thereof comprise one or more CH3 comprising an amino acid sequence that is at least 65%, at least 70%, at least 75%, at least 80%, at least 85%, at least 90%, at least 95%, at least 99%, or 100% identical to any one of SEQ ID NOs: 1079-1085.

In some aspects, the antigen binding polypeptide or antigen binding polypeptide complex, the antibody or antigen binding fragment thereof, the polypeptide, the anti-SARS-CoV-2 virus antibody or antigen binding fragment thereof, comprises one or more immunoglobulin hinge, and the one or more immunoglobulin hinge comprises an amino acid sequence that is at least 65%, at least 70%, at least 75%, at least 80%, at least 85%, at least 90%, at least 95%, at least 99%, or 100% identical to any one of SEQ ID NOs: 635, 636, 1072, 1073, or 1074.

Also provided herein is an antigen binding polypeptide having a structure, from amino-terminus to carboxy-terminus, represented by: VL1-L1-CL-L2-VL2-L3-VH2-L4-VH1-L5-CH1; VL1-L1-CL-L2-VH2-L3-VL2-L4-VH1-L5-CH1; VL1-L1-CH1-L2-VL2-L3-VH2-L4-VH1-L5-CL; VL1-L1-CH1-L2- VH2-L3-VL2-L4-VH1-L5-CL; VH1-L1-CL-L2-VL2-L3-VH2-L4-VL1-L5-CH1; VH1-L1-CL-L2-VH2-L3-VL2-L4-VL1-L5-CH1; VH1-L1-CH1-L2-VL2-L3-VH2-L4-VL1-L5-CL; VH1-L1-CH1-L2-VH2-L3- VL2-L4-VL1-L5-CL; VL2-L1-CL-L2-VL1-L3-VH1-L4-VH2-L5-CH1; VL2-L1-CL-L2-VH1-L3-VL1-L4-VH2-L5-CH1; VL2-L1-CH1-L2-VL1-L3-VH1-L4-VH2-L5-CL; VL2-L1-CH1-L2-VH1-L3-VL1-L4-VH2-L5-CL; VH2-L1-CL-L2-VL1-L3-VH1-L4-VL2-L5-CH1; VH2-L1-CL-L2-VH1-L3-VL1-L4-VL2-L5-CH1; VH2-L1-CH1-L2-VL1-L3-VH1-L4-VL2-L5-CL; or VH2-L1-CH1-L2-VH1-L3-VL1-L4-VL2-L5-CL; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L5 are optional amino acid linkers.

Also provided herein is an antigen binding polypeptide having a structure, from amino-terminus to carboxy-terminus, represented by: VL1-CL-L1-VL2-L2-VH2-L3-VH1-CH1; VL1-CL-L1-VH2-L2-VL2-L3-VH1-CH1; VL1-CH1-L1-VL2-L2-VH2-L3-VH1-CL; VL1-CH1-L1-VH2-L2-VL2-L3-VH1-CL; VH1-CL-L1-VL2-L2-VH2-L3-VL1-CH1; VH1-CL-L1-VH2-L2-VL2-L3-VL1-CH1; VH1-CH1-L1-VL2-L2-VH2-L3-VL1-CL; VH1-CH1-L1-VH2-L2-VL2-L3-VL1-CL; VL2-CL-L1-VL1-L2-VH1-L3-VH2-CH1; VL2-CL-L1-VH1-L2-VL1-L3-VH2-CH1; VL2-CH1-L1-VL1-L2-VH1-L3-VH2-CL; VL2-CH1-L1-VH1-L2-VL1-L3-VH2-CL; VH2-CL-L1-VL1-L2-VH1-L3-VL2-CH1; VH2-CL-L1-VH1-L2-VL1-L3-VL2-CH1; VH2-CH1-L1-VL1-L2-VH1-L3-VL2-CL; or VH2-CH1-L1-VH1-L2-VL1-L3-VL2-CL; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L3 are amino acid linkers.

Also provided herein is an antigen binding polypeptide having a structure, from amino-terminus to carboxy-terminus, represented by: VL1-L1-VH1-L2-VL2-L3-CL-L4-VH2-L5-CH1; VL1-L1-VH1-L2-VL2-L3-CH1-L4-VH2-L5-CL; VL1-L1-VH1-L2-VH2-L3-CL-L4-VL2-L5-CH1; VL1-L1-VH1-L2-VH2-L3-CH1-L4-VL2-L5-CL; VL1-L1-VL2-L2-VH1-L3-CL-L4-VH2-L5-CH1; VL1-L1-VL2-L2-VH1-L3-CH1-L4-VH2-L5-CL; VL1-L1-VH2-L2-VH1-L3-CL-L4-VL2-L5-CH1; VL1-L1-VH2-L2-VH1-L3-CH1-L4-VL2-L5-CL; VH1-L1-VL1-L2-VL2-L3-CL-L4-VH2-L5-CH1; VH1-L1-VL1-L2-VL2-L3-CH1-L4-VH2-L5-CL; VH1-L1-VL1-L2-VH2-L3-CL-L4-VL2-L5-CH1; VH1-L1-VL1-L2-VH2-L3-CH1-L4-VL2-L5-CL; VH1-L1-VL2-L2-VL1-L3-CL-L4-VH2-L5-CH1; VH1-L1-VL2-L2-VL1-L3-CH1-L4-VH2-L5-CL; VH1-L1-VH2-L2-VL1-L3-CL-L4-VL2-L5-CH1; VH1-L1-VH2-L2-VL1-L3-CH1-L4-VL2-L5- CL; VL2-L1-VL1-L2-VH2-L3-CL-L4-VH1-L5-CH1; VL2-L1-VL1-L2-VH2-L3-CH1-L4-VH1-L5-CL; VL2-L1-VH1-L2-VH2-L3-CL-L4-VL1-L5-CH1; VL2-L1-VH1-L2-VH2-L3-CH1-L4-VL1-L5-CL; VL2-L1-VH2-L2-VL1-L3-CL-L4-VH1-L5-CH1; VL2-L1-VH2-L2-VL1-L3-CH1-L4-VH1-L5-CL; VL2-L1-VH2-L2-VH1-L3-CL-L4-VL1-L5-CH1; VL2-L1-VH2-L2-VH1-L3-CH1-L4-VL1-L5-CL; VH2-L1-VL1-L2-VL2-L3-CL-L4-VH1-L5-CH1; VH2-L1-VL1-L2-VL2-L3-CH1-L4-VH1-L5-CL; VH2-L1-VH1-L2-VL2-L3-CL-L4-VL1-L5-CH1; VH2-L1-VH1-L2-VL2-L3-CH1-L4-VL1-L5-CL; VH2-L1-VL2-L2-VL1-L3-CL-LA-VH1-L5-CH1; VH2-L1-VL2-L2-VL1-L3-CH1-L4-VH1-L5-CL; VH2-L1-VL2-L2-VH1-L3-CL-L4- VL1-L5-CH1; or VH2-L1-VL2-L2-VH1-L3-CH1-L4-VL1-L5-CL; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to a (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L5 are optional amino acid linkers.

Also provided herein is an antigen binding polypeptide having a structure, from amino-terminus to carboxy-terminus, represented by: VL1-L1-CL-L2-VH1-L3-CH1-L4-VL2-L5-VH2; VL1-L1-CL-L2-VH1-L3-CH1-L4-VH2-L5-VL2; VL1-L1-CL-L2-VL2-L3-CH1-L4-VH1-L5-VH2; VL1-L1-CL-L2-VH2-L3-CH1-L4-VH1-L5-VL2; VL1-L1-CH1-L2-VH1-L3-CL-L4-VL2-L5-VH2; VL1-L1-CH1-L2-VH1-L3-CL-L4-VH2-L5-VL2; VL1-L1-CH1-L2-VL2-L3-CL-L4-VH1-L5-VH2; VL1-L1-CH1-L2-VH2-L3-CL- L4-VH1-L5-VL2; VH1-L1-CL-L2-VL1-L3-CH1-L4-VL2-L5-VH2; VH1-L1-CL-L2-VL1-L3-CH1-L4-VH2-L5-VL2; VH1-L1-CL-L2-VL2-L3-CH1-L4-VL1-L5-VH2; VH1-L1-CL-L2-VH2-L3-CH1-L4-VL1-L5-VL2; VH1-L1-CH1-L2-VL1-L3-CL-L4-VL2-L5-VH2; VH1-L1-CH1-L2-VL1-L3-CL-L4-VH2-L5-VL2; VH1-L1-CH1-L2-VL2-L3-CL-L4-VL1-L5-VH2; VH1-L1-CH1-L2-VH2-L3-CL-L4-VL1-L5-VL2; VL2-L1-CL-L2-VL1-L3-CH1-L4-VH2-L5-VH1; VL2-L1-CL-L2-VH1-L3-CH1-L4-VH2-L5-VL1; VL2-L1-CL-L2-VH2-L3-CH1-L4-VL1-L5-VH1; VL2-L1-CL-L2-VH2-L3-CH1-L4-VH1-L5-VL1; VL2-L1-CH1-L2-VL1-L3-CL-L4-VH2-L5-VH1; VL2-L1-CH1-L2-VH1-L3-CL-L4-VH2-L5-VL1; VL2-L1-CH1-L2-VH2-L3-CL-L4-VL1-L5-VH1; VL2-L1-CH1-L2-VH2-L3-CL-L4-VH1-L5-VL1; VH2-L1-CL-L2-VL1-L3-CH1-L4-VL2-L5-VH1; VH2-L1-CL-L2-VH1-L3-CH1-L4-VL2-L5-VL1; VH2-L1-CL-L2-VL2-L3-CH1-L4-VL1-L5-VH1; VH2-L1-CL-L2-VL2-L3-CH1-L4-VH1-L5-VL1; VH2-L1-CH1-L2-VL1-L3-CL-L4-VL2-L5-VH1; VH2-L1-CH1-L2-VH1-L3-CL-L4-VL2-L5-VL1; VH2-L1-CH1-L2-VL2-L3-CL-L4-VL1-L5-VH1; or VH2-L1-CH1-L2-VL2-L3-CL-L4-VH1-L5-VL1; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L5 are optional amino acid linkers.

Also provided herein is an antigen binding polypeptide having a structure, from amino-terminus to carboxy-terminus, represented by: VL1-L1-CL-L2-VH1-L3-CH1; VL1-L1-CH1-L2-VH1-L3-CL; VH1-L1-CL-L2-VL1-L3-CH1; or VH1-L1-CH1-L2-VL1-L3-CL; wherein: VL1 is an immunoglobulin light chain variable region that specifically binds to (i) a tumor-associated antigen (TAA), (ii) an infectious disease antigen that is not a sarbecovirus antigen, or (iii) an other-pathology antigen; VH1 is an immunoglobulin heavy chain variable region that specifically binds to a (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L3 are optional amino acid linkers.

Also provided herein is an antigen binding polypeptide having a structure, from amino-terminus to carboxy-terminus, represented by: VL1-L1-VH1-L2-CL; or VH1-L1-VL1-L2-CL; wherein: VL1 is an immunoglobulin light chain variable region that specifically binds to (i) a tumor-associated antigen (TAA), (ii) an infectious disease antigen that is not a sarbecovirus antigen, or (iii) an other-pathology antigen; VH1 is an immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; and L1-L2 are optional amino acid linkers.

Also provided herein is an antigen binding polypeptide having a structure, from amino-terminus to carboxy-terminus, represented by: VL1-L1-VH1-L2-CH1; or VH1-L1-VL1-L2-CH1; wherein: VL1 is an immunoglobulin light chain variable region that specifically binds to (i) a tumor-associated antigen (TAA), (ii) an infectious disease antigen that is not a sarbecovirus antigen, or (iii) an other-pathology antigen; VH1 is an immunoglobulin heavy chain variable region that specifically binds to (i) a tumor-associated antigen (TAA), (ii) an infectious disease antigen that is not a sarbecovirus antigen, or (iii) an other-pathology antigen; CH1 is an immunoglobulin heavy chain constant region 1; and L1-L2 are optional amino acid linkers.

Also provided herein is an antigen binding polypeptide having a structure, from amino-terminus to carboxy-terminus, represented by: VL1-L1-VL2-L2-CH1; or VL2-L1-VL1-L2-CH1; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; CH1 is an immunoglobulin heavy chain constant region 1; and L1-L2 are optional amino acid linkers.

Also provided herein is an antigen binding polypeptide having a structure, from amino-terminus to carboxy-terminus, represented by: VH1-L1-VH2-L2-CL; or VH2-L1-VH1-L2-CL; wherein: VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; and L1-L2 are optional amino acid linkers.

Also provided herein is an antigen binding polypeptide having a structure, from amino-terminus to carboxy-terminus, represented by: VL1-L1-VL2-L2-VL3-L3-CH1; VL1-L1-VL3-L2-VL2-L3-CH1; VL2-L1-VL1-L2-VL3-L3-CH1; VL2-L1-VL3-L2-VL1-L3-CH1; VL3-L1-VL1-L2-VL2-L3-CH1; or VL3-L1-VL2-L2-VL1-L3-CH1; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; CH1 is an immunoglobulin heavy chain constant region 1; and L1-L3 are optional amino acid linkers.

Also provided herein is an antigen binding polypeptide having a structure, from amino-terminus to carboxy-terminus, represented by: VH1-L1-VH2-L2-VH3-L3-CL; VH1-L1-VH3-L2-VH2-L3-CL; VH2-L1-VH1-L2-VH3-L3-CL; VH2-L1-VH3-L2-VH1-L3-CL; VH3-L1-VH1-L2-VH2-L3-CL; or VH3-L1-VH2-L2-VH1-L3-CL; wherein: VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; and L1-L3 are optional amino acid linkers.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgen receptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSF1R, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-related antigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10), IL12, IL13, IL13Ra2, IL-13Ra2, IL13Ra1, IL15, IL17, IL-17A, IL-17AF, IL-17F, IL17Rb, IL18, IL1B, IL1F10, IL-1α, IL-1β, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-11Ra, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70)-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL, Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRP1 glycoprotein 75, UPK2, VAP-1, VEGF, VEGFA, VEGFR-1, VEGFR2, vimentin, VISTA, Vstm3, WTI, WUCAM, and XAGE1.

In some aspects, and in any one of the formulas depicted herein, any one or more of (i) VL1 and VH1, (ii) VL2 and VH2, and (iii) VL3 and VH3, comprise a light chain complementarity determining region 1 (LCDR1), LCDR2, and/or LCDR3, and a heavy chain complementarity determining region 1 (HCDR1). HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, cnapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140), rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, cramkafusp alfa, ripertamab, zuberitamab, glofitamab, nofetumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmolcukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxctan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650), tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, clotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, and in any one of the formulas depicted herein, any one or more of (i) VL1 and VH1, (ii) VL2 and VH2, and (iii) VL3 and VH3, comprise a VL and a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, cculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140), rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, cramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirinc, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, cpitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650), tralokinumab, anrukinzumab, brodalumab, ixckizumab, netakimab, perakizumab, secukinumab, vunakizumab, afasevikumab, afasevikumab, bimckizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, and in any one of the formulas depicted herein, (a) any one or more of VL1, VL2, and VL3 comprises: (i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a LCDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777, or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976; and (b) any one or more of VH1, VH2, and VH3 comprises: (i) a HCDR1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a HCDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, and in any one of the formulas depicted herein, (a) any one or more of VL1, VL2, and VL3 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788; and (b) any one or more of VH1, VH2, and VH3 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is: (i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2). Yamagata virus antigen. Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1. HIV envelope glycoprotein (Env). HIV envelope glycoprotein gp160. HIV envelope surface glycoprotein gp120. HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or (ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium. Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, and in any one of the formulas depicted herein, any one or more of (i) VL1 and VH1, (ii) VL2 and VH2, and (iii) VL3 and VH3, comprise a light complementarity determining region 1 (LCDR1). LCDR2, and/or LCDR3, and a heavy complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab. REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab. CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, and in any one of the formulas depicted herein, any one or more of (i) VL1 and VH1, (ii) VL2 and VH2, and (iii) VL3 and VH3, comprise a VL and a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, and in any one of the formulas depicted herein, (a) any one or more of VL1, VL2, and VL3 comprises: (i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a LCDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002, or at least 65%, at least 70%, at least 75%, at least 80%, at least 85%, at least 90%, at least 95%, at least 99%, or 100% identical to any one of amino acid sequences YTS, DTS, GAS, and AAS; and (iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003; and (b) any one or more of VH1, VH2, and VH3 comprises: (i) a HCDR1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a HCDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, and in any one of the formulas depicted herein, (a) any one or more of VL1, VL2, and VL3 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004; and (b) any one or more of VH1, VH2, and VH3 comprises an amino acid sequence that is at least 65%, at least 70%, at least 75%, at least 80%, at least 85%, at least 90%, at least 95%, at least 99%, or 100% identical to any one of SEQ ID NOs: 1797, 1804, 1809, 1816, 1823, 1830, 1984, 1992, 2000, and 2008.

In some aspects, the other-pathology antigen is selected from the group consisting of Amyloid beta, β-amyloid, PCSK9), IFN-α/β receptor, Rhesus factor, nerve growth factor (NGF), DPP4, fibrin II beta chain, ACVR2B, serum amyloid A protein SOST, FGF 23, VWF, tissue factor pathway inhibitor (TFPI), 1-40) and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40) and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, and in any one of the formulas depicted herein, any one or more of (i) VL1 and VH1, (ii) VL2 and VH2, and (iii) VL3 and VH3, comprise a light complementarity determining region 1 (LCDR1). LCDR2, and/or LCDR3, and a heavy complementarity determining region 1 (HCDR1). HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, and in any one of the formulas depicted herein, any one or more of (i) VL1 and VH1, (ii) VL2 and VH2, and (iii) VL3 and VH3, comprise the VL and the VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, and in any one of the formulas depicted herein, (a) any one or more of VL1, VL2, and VL3 comprises: (i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a LCDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970; and (b) any one or more of VH1, VH2, and VH3 comprises: (i) a HCDR1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a HCDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, and in any one of the formulas depicted herein, (a) any one or more of VL1, VL2, and VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971; and (b) any one or more of VH1, VH2, and VH3 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

Also provided herein is an antigen binding polypeptide having a structure, from amino-terminus to carboxy-terminus, represented by: VL1-L1-VL2-L2-VH2-L3-VH1-L4-CL-L5-CH1; VL1-L1-VL2-L2-VH2-L3-VH1-L4-CH1-L5-CL; VL1-L1-VH2-L2-VL2-L3-VH1-L4-CL-L5-CH1; VL1-L1-VH2-L2-VL2-L3-VH1-L4-CH1-L5-CL; VH1-L1-VL2-L2-VH2-L3-VL1-L4-CL-L5-CH1; VH1-L1-VL2-L2-VH2-L3-VL1-L4-CH1-L5-CL; VH1-L1-VH2-L2-VL2-L3-VL1-L4-CL-L5-CH1; VH1-L1-VH2-L2-VL2-L3-VL1-L4-CH1-L5-CL; VL2-L1-VL1-L2-VH1-L3-VH2-L4-CL-L5-CH1; VL2-L1-VL1-L2-VH1-L3-VH2-L4-CH1-L5-CL; VL2-L1-VH1-L2-VL1-L3-VH2-L4-CL-L5-CH1; VL2-L1-VH1-L2-VL1-L3-VH2-L4-CH1-L5-CL; VH2-L1-VL1-L2-VH1-L3-VL2-L4-CL-L5-CH1; VH2-L1-VL1-L2-VH1-L3-VL2-L4-CH1-L5-CL; VH2-L1-VH1-L2-VL1-L3-VL2-L4-CL-L5-CH1; or VH2-L1-VH1-L2-VL1-L3-VL2-L4-CH1-L5-CL; wherein: (a) VL1 is a first immunoglobulin light chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786, a CDR2 having an amino acid sequence at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS or an amino acid sequence at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777, and a CDR3 having an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976; VL2 is a second immunoglobulin light chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786, a CDR2 having an amino acid sequence at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS or an amino acid sequence at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777, and a CDR3 having an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976; VH1 is a first immunoglobulin heavy chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789, a CDR2 having an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790, and a CDR3 having an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and VH2 is a second immunoglobulin heavy chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789, a CDR2 having an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790, and a CDR3 having an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; (b) VL1 is a first immunoglobulin light chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001, a CDR2 having an amino acid sequence at least 65%, at least 70%, at least 75%, at least 80%, at least 85%, at least 90%, at least 95%, at least 99%, or 100% identical to any one of amino acid sequences YTS, DTS, GAS, and AAS or an amino acid sequence at least 65%, at least 70%, at least 75%, at least 80%, at least 85%, at least 90%, at least 95%, at least 99%, or 100% identical to any one of SEQ ID NOs: 1978, 1986, 1994, and 2002, and a CDR3 having an amino acid sequence at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003; VL2 is a second immunoglobulin light chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001, a CDR2 having an amino acid sequence at least 65%, at least 70%, at least 75%, at least 80%, at least 85%, at least 90%, at least 95%, at least 99%, or 100% identical to any one of amino acid sequences YTS, DTS, GAS, and AAS or an amino acid sequence at least 65%, at least 70%, at least 75%, at least 80%, at least 85%, at least 90%, at least 95%, at least 99%, or 100% identical to any one of SEQ ID NOs: 1978, 1986, 1994, and 2002, and a CDR3 having an amino acid sequence at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003; VH1 is a first immunoglobulin heavy chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005, a CDR2 having an amino acid sequence at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006, and a CDR3 having an amino acid sequence at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and VH2 is a second immunoglobulin heavy chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005, a CDR2 having an amino acid sequence at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006, and a CDR3 having an amino acid sequence at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or (c) VL1 is a first immunoglobulin light chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963, a CDR2 having an amino acid sequence at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS, and a CDR3 having an amino acid sequence at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970; VL2 is a second immunoglobulin light chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963, a CDR2 having an amino acid sequence at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS, and a CDR3 having an amino acid sequence at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970; VH1 is a first immunoglobulin heavy chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966, a CDR2 having an amino acid sequence at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972, and a CDR3 having an amino acid sequence at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and VH2 is a second immunoglobulin heavy chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966, a CDR2 having an amino acid sequence at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972, and a CDR3 having an amino acid sequence at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and wherein: CL is an immunoglobulin light chain constant region; CH1 is an immunoglobulin heavy chain constant region 1; and L1-L15 are optional amino acid linkers.

In some aspects, and in any one of the formulas depicted herein, (a) VL1 comprises an amino acid sequence that is at least 65%, at least 70%, at least 75%, at least 80%, at least 85%, at least 90%, at least 95%, at least 99%, or 100% identical to SEQ ID NO: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788; VL2 comprises an amino acid sequence that is at least 65%, at least 70%, at least 75%, at least 80%, at least 85%, at least 90%, at least 95%, at least 99%, or 100% identical to SEQ ID NO: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788; VH1 comprises an amino acid sequence that is at least 65%, at least 70%, at least 75%, at least 80%, at least 85%, at least 90%, at least 95%, at least 99%, or 100% identical to SEQ ID NO: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and VH2 comprises an amino acid sequence that is at least 65%, at least 70%, at least 75%, at least 80%, at least 85%, at least 90%, at least 95%, at least 99%, or 100% identical to SEQ ID NO: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; (b) VL1 comprises an amino acid sequence that is at least 65%, at least 70%, at least 75%, at least 80%, at least 85%, at least 90%, at least 95%, at least 99%, or 100% identical to SEQ ID NO: 1794, 1800, 1806, 1812, 1819, 1826, 1980, 1988, 1996, and 2004; VL2 comprises an amino acid sequence that is at least 65%, at least 70%, at least 75%, at least 80%, at least 85%, at least 90%, at least 95%, at least 99%, or 100% identical to SEQ ID NO: 1794, 1800, 1806, 1812, 1819, 1826, 1980, 1988, 1996, and 2004; VH1 comprises an amino acid sequence that is at least 65%, at least 70%, at least 75%, at least 80%, at least 85%, at least 90%, at least 95%, at least 99%, or 100% identical to SEQ ID NO: 1797, 1804, 1809, 1816, 1823, 1830, 1984, 1992, 2000, and 2008; and VH2 comprises an amino acid sequence that is at least 65%, at least 70%, at least 75%, at least 80%, at least 85%, at least 90%, at least 95%, at least 99%, or 100% identical to SEQ ID NO: 1797, 1804, 1809, 1816, 1823, 1830, 1984, 1992, 2000, and 2008; or (c) VL1 comprises an amino acid sequence that is at least 65%, at least 70%, at least 75%, at least 80%, at least 85%, at least 90%, at least 95%, at least 99%, or 100% identical to SEQ ID NO: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971; VL2 comprises an amino acid sequence that is at least 65%, at least 70%, at least 75%, at least 80%, at least 85%, at least 90%, at least 95%, at least 99%, or 100% identical to SEQ ID NO: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971; VH1 comprises an amino acid sequence that is at least 65%, at least 70%, at least 75%, at least 80%, at least 85%, at least 90%, at least 95%, at least 99%, or 100% identical to SEQ ID NO: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and VH2 comprises an amino acid sequence that is at least 65%, at least 70%, at least 75%, at least 80%, at least 85%, at least 90%, at least 95%, at least 99%, or 100% identical to SEQ ID NO: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, and in any one of the formulas depicted herein, the CL comprises an amino acid sequence that is at least 65%, at least 70%, at least 75%, at least 80%, at least 85%, at least 90%, at least 95%, at least 99%, or 100% identical to any one of SEQ ID NOs: 374, 637, or 1092-1095. In some aspects, and in any one of the formulas depicted herein, the CH1 comprises an amino acid sequence that is at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptide further comprises an immunoglobulin heavy chain constant region 2 (CH2), and wherein the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the antigen binding polypeptide further comprises an immunoglobulin heavy chain constant region 3 (CH3), and wherein the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085.

In some aspects, the antigen binding polypeptide further comprises an Fc region at the carboxy-terminus. In some aspects, the Fc region comprises an immunoglobulin heavy chain constant region 2 (CH2) and/or an immunoglobulin heavy chain constant region 3 (CH3). In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085.

In some aspects, the Fc region further comprises an immunoglobulin hinge. In some aspects, the immunoglobulin hinge comprises an amino acid sequence at least 65%, at least 70%, at least 75%, at least 80%, at least 85%, at least 90%, at least 95%, at least 99%, or 100% identical to any one of SEQ ID NOS: 635.636, 1072, 1073, or 1074.

In some aspects, the Fc region comprises one or more half-life extension amino acid substitutions, one or more knob-into-hole modification, one or more Fc effector function knockout mutation, or any combination thereof. In some aspects, the Fc region is an IgG Fc region. In some aspects, the Fc region is an IgG1 or an IgG4 Fc region. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme.

In some aspects, the one or more half-life extension amino acid substitutions comprise amino acid substitutions: (i) L234A and L235A (LA); (ii) M428L and N434S (LS); (iii) L234F and L235E; (iv) L234F, L235E, and P331S (TM); and/or (v) M252Y, S254T, and T256E (YTE); or equivalent thereof, based on the EU numbering scheme.

In some aspects, the one or more the knob-into-hole modification comprises: (i) knob substitutions of S354C and T366W; (ii) hole substitutions of Y349C, T366S, L368A and Y407V; or (iii) a combination thereof; or equivalent thereof, based on the EU numbering scheme. In some aspects, the one or more Fc effector function knockout mutation is L234A, L235A, P239A, or a combination thereof, or equivalent thereof, based on the EU numbering scheme.

In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VL2-L3-VH2-L4-VH1-L5-CH1; VL1-L1-CL-L2-VH2-L3-VL2-L4-VH1-L5-CH1; VL1-L1-CH1-L2-VL2-L3-VH2-L4-VH1-L5-CL; VL1-L1-CH1-L2-VH2-L3- VL2-L4-VH1-L5-CL; VH1-L1-CL-L2-VL2-L3-VH2-L4-VL1-L5-CH1; VH1-L1-CL-L2-VH2-L3-VL2-L4-VL1-L5-CH1; VH1-L1-CH1-L2-VL2-L3-VH2-L4-VL1-L5-CL; VH1-L1-CH1-L2-VH2-L3-VL2-L4- VL1-L5-CL; VL2-L1-CL-L2-VL1-L3-VH1-L4-VH2-L5-CH1; VL2-L1-CL-L2-VH1-L3-VL1-L4-VH2-L5-CH1; VL2-L1-CH1-L2-VL1-L3-VH1-L4-VH2-L5-CL; VL2-L1-CH1-L2-VH1-L3-VL1-L4-VH2-L5- CL; VH2-L1-CL-L2-VL1-L3-VH1-L4-VL2-L5-CH1; VH2-L1-CL-L2-VH1-L3-VL1-L4-VL2-L5-CH1; VH2-L1-CH1-L2-VL1-L3-VH1-L4-VL2-L5-CL; or VH2-L1-CH1-L2-VH1-L3-VL1-L4-VL2-L5-CL; wherein the second polypeptide has a structure, from amino-terminus to carboxy-terminus, represented by: VL3-L6-CL-L7-VL4-L8-VH4-L9-VH3-L10-CH1; VL3-L6-CL-L7-VH4-L8-VL4-L9-VH3-L10-CH1; VL3-L6- CH1-L7-VL4-L8-VH4-L9-VH3-L10-CL; VL3-L6-CH1-L7-VH4-L8-VL4-L9-VH3-L10-CL; VH3-L6-CL-L7- VL4-L8-VH4-L9-VL3-L10-CH1; VH3-L6-CL-L7-VH4-L8-VL4-L9-VL3-L10-CH1; VH3-L6-CH1-L7-VL4-L8-VH4-L9-VL3-L10-CL; VH3-L6-CH1-L7-VH4-L8-VL4-L9-VL3-L10-CL; VL4-L6-CL-L7-VL3-L8- VH3-L9-VH4-L10-CH1; VL4-L6-CL-L7-VH3-L8-VL3-L9-VH4-L10-CH1; VL4-L6-CH1-L7-VL3-L8-VH3-L9-VH4-L10-CL; VL4-L6-CH1-L7-VH3-L8-VL3-L9-VH4-L10-CL; VH4-L6-CL-L7-VL3-L8-VH3-L9- VL4-L10-CH1; VH4-L6-CL-L7-VH3-L8-VL3-L9-VL4-L10-CH1; VH4-L6-CH1-L7-VL3-L8-VH3-L9-VL4-L10-CL; or VH4-L6-CH1-L7-VH3-L8-VL3-L9-VL4-L10-CL; and wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L10 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-CL-L1-VL2-L2-VH2-L3-VH1-CH1; VL1-CL-L1-VH2-L2-VL2-L3-VH1-CH1; VL1-CH1-L1-VL2-L2-VH2-L3-VH1-CL; VL1-CH1-L1-VH2-L2-VL2-L3-VH1-CL; VH1-CL-L1-VL2-L2-VH2-L3-VL1-CH1; VH1-CL-L1-VH2-L2-VL2-L3-VL1-CH1; VH1-CH1-L1-VL2-L2-VH2-L3-VL1-CL; VH1-CH1-L1-VH2-L2-VL2-L3-VL1-CL; VL2-CL-L1-VL1-L2-VH1-L3-VH2-CH1; VL2-CL-L1-VH1-L2-VL1-L3-VH2-CH1; VL2-CH1-L1-VL1-L2-VH1-L3-VH2-CL; VL2-CH1-L1-VH1-L2- VL1-L3-VH2-CL; VH2-CL-L1-VL1-L2-VH1-L3-VL2-CH1; VH2-CL-L1-VH1-L2-VL1-L3-VL2-CH1; VH2-CH1-L1-VL1-L2-VH1-L3-VL2-CL; or VH2-CH1-L1-VH1-L2-VL1-L3-VL2-CL; wherein the second polypeptide has a structure, from amino-terminus to carboxy-terminus, represented by: VL3-CL-L4-VL4-L5-VH4-L6-VH3-CH1; VL3-CL-L4-VH4-L5-VL4-L6-VH3-CH1; VL3-CH1-L4-VL4-L5-VH4-L6-VH3-CL; VL3-CH1-L4-VH4-L5-VL4-L6-VH3-CL; VH3-CL-L4-VL4-L5-VH4-L6-VL3-CH1; VH3-CL-L4-VH4-L5-VL4-L6-VL3-CH1; VH3-CH1-L4-VL4-L5-VH4-L6-VL3-CL; VH3-CH1-L4-VH4-L5-VL4-L6-VL3-CL; VL4-CL-L4-VL3-L5-VH3-L6-VH4-CH1; VL4-CL-L4-VH3-L5-VL3-L6-VH4-CH1; VL4-CH1-L4-VL3-L5-VH3-L6-VH4-CL; VL4-CH1-L4-VH3-L5-VL3-L6-VH4-CL; VH4-CL-L4-VL3-L5-VH3- L6-VL4-CH1; VH4-CL-L4-VH3-L5-VL3-L6-VL4-CH1; VH4-CH1-L4-VL3-L5-VH3-L6-VL4-CL; or VH4-CH1-L4-VH3-L5-VL3-L6-VL4-CL; and wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L6 are amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-VH1-L2-VL2-L3-CL-L4-VH2-L5-CH1; VL1-L1-VH1-L2-VL2-L3-CH1-L4-VH2-L5-CL; VL1-L1-VH1-L2-VH2-L3-CL-L4-VL2-L5-CH1; VL1-L1-VH1-L2-VH2-L3-CH1-L4-VL2-L5-CL; VL1-L1-VL2-L2-VH1-L3-CL-L4-VH2-L5-CH1; VL1-L1-VL2-L2-VH1-L3-CH1-L4-VH2-L5-CL; VL1-L1-VH2-L2-VH1-L3-CL-L4-VL2-L5-CH1; VL1-L1-VH2-L2-VH1-L3-CH1-L4-VL2-L5-CL; VH1-L1-VL1-L2-VL2-L3-CL-L4-VH2-L5-CH1; VH1-L1-VL1-L2-VL2-L3-CH1-L4-VH2-L5-CL; VH1-L1-VL1-L2-VH2-L3-CL-L4-VL2-L5-CH1; VH1-L1-VL1-L2-VH2-L3-CH1-L4-VL2-L5- CL; VH1-L1-VL2-L2-VL1-L3-CL-L4-VH2-L5-CH1; VH1-L1-VL2-L2-VL1-L3-CH1-L4-VH2-L5-CL; VH1-L1-VH2-L2-VL1-L3-CL-L4-VL2-L5-CH1; VH1-L1-VH2-L2-VL1-L3-CH1-L4-VL2-L5-CL; VL2-L1-VL1-L2-VH2-L3-CL-L4-VH1-L5-CH1; VL2-L1-VL1-L2-VH2-L3-CH1-L4-VH1-L5-CL; VL2-L1-VH1-L2-VH2-L3-CL-L4-VL1-L5-CH1; VL2-L1-VH1-L2-VH2-L3-CH1-L4-VL1-L5-CL; VL2-L1-VH2-L2-VL1-L3-CL-L4-VH1-L5-CH1; VL2-L1-VH2-L2-VL1-L3-CH1-L4-VH1-L5-CL; VL2-L1-VH2-L2-VH1-L3-CL-L4-VL1-L5-CH1; VL2-L1-VH2-L2-VH1-L3-CH1-L4-VL1-L5-CL; VH2-L1-VL1-L2-VL2-L3-CL-L4-VH1-L5-CH1; VH2-L1-VL1-L2-VL2-L3-CH1-L4-VH1-L5-CL; VH2-L1-VH1-L2-VL2-L3-CL-L4-VL1-L5-CH1; VH2-L1-VH1-L2-VL2-L3-CH1-L4-VL1-L5-CL; VH2-L1-VL2-L2-VL1-L3-CL-L4-VH1-L5-CH1; VH2-L1-VL2-L2-VL1-L3-CH1-L4-VH1-L5-CL; VH2-L1-VL2-L2-VH1-L3-CL-L4-VL1-L5-CH1; or VH2-L1-VL2-L2-VH1-L3-CH1-L4-VL1-L5-CL; wherein the second polypeptide has a structure, from amino-terminus to carboxy-terminus, represented by: VL3-L6-VH3-L7-VL4-L8-CL-L9-VH4-L10-CH1; VL3-L6-VH3-L7-VL4-L8-CH1-L9-VH4-L10-CL; VL3-L6-VH3-L7-VH4-L8-CL-L9-VL4-L10-CH1; VL3-L6-VH3-L7-VH4-L8-CH1-L9-VL4-L10-CL; VL3-L6-VL4-L7-VH3-L8-CL-L9-VH4-L10-CH1; VL3-L6-VL4-L7-VH3-L8-CH1-L9-VH4-L10-CL; VL3-L6-VH4-L7-VH3-L8-CL-L9-VL4-L10-CH1; VL3-L6-VH4-L7-VH3-L8-CH1-L9-VL4-L10-CL; VH3-L6-VL3-L7-VL4-L8-CL-L9-VH4-L10-CH1; VH3-L6-VL3-L7-VL4-L8-CH1-L9-VH4-L10-CL; VH3-L6-VL3-L7-VH4-L8-CL-L9-VL4-L10-CH1; VH3-L6-VL3-L7-VH4-L8-CH1-L9-VL4-L10-CL; VH3-L6-VL4-L7-VL3-L8-CL-L9-VH4-L10-CH1; VH3-L6-VL4-L7-VL3-L8-CH1-L9-VH4-L10-CL; VH3-L6-VH4-L7-VL3-L8-CL-L9-VL4-L10-CH1; VH3-L6-VH4-L7-VL3-L8-CH1-L9-VL4-L10-CL; VL4-L6-VL3-L7-VH4-L8-CL-L9-VH3-L10-CH1; VL4-L6-VL3-L7-VH4-L8-CH1-L9-VH3-L10-CL; VL4-L6-VH3-L7-VH4-L8-CL-L9-VL3-L10-CH1; VL4-L6-VH3-L7-VH4-L8-CH1-L9-VL3-L10-CL; VL4-L6-VH4-L7-VL3-L8-CL-L9-VH3-L10-CH1; VL4-L6-VH4-L7-VL3-L8-CH1-L9-VH3-L10-CL; VL4-L6-VH4-L7-VH3-L8-CL-L9-VL3-L10-CH1; VL4-L6-VH4-L7-VH3-L8-CH1-L9-VL3-L10-CL; VH4-L6-VL3-L7-VL4-L8-CL-L9-VH3-L10-CH1; VH4-L6-VL3-L7-VL4-L8-CH1-L9-VH3-L10-CL; VH4-L6-VH3-L7-VL4-L8-CL-L9-VL3-L10-CH1; VH4-L6-VH3-L7-VL4-L8-CH1-L9-VL3-L10-CL; VH4-L6-VL4-L7-VL3-L8-CL-L9-VH3-L10-CH1; VH4-L6-VL4-L7-VL3-L8-CH1-L9-VH3-L10-CL; VH4-L6-VL4-L7-VH3-L8-CL-L9-VL3-L10-CH1; or VH4-L6-VL4-L7-VH3-L8-CH1-L9-VL3-L10-CL; and wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L10 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VH1-L3-CH1-L4-VL2-L5-VH2; VL1-L1-CL-L2-VH1-L3-CH1-L4-VH2-L5-VL2; VL1-L1-CL-L2-VL2-L3-CH1-L4-VH1-L5-VH2; VL1-L1-CL-L2-VH2-L3-CH1-L4-VH1-L5-VL2; VL1-L1-CH1-L2-VH1-L3-CL-L4-VL2-L5-VH2; VL1-L1-CH1-L2-VH1-L3-CL-L4-VH2-L5-VL2; VL1-L1-CH1-L2-VL2-L3-CL-L4-VH1-L5-VH2; VL1-L1-CH1-L2-VH2-L3-CL-L4-VH1-L5-VL2; VH1-L1-CL-L2-VL1-L3-CH1-L4-VL2-L5-VH2; VH1-L1-CL-L2-VL1-L3-CH1-L4-VH2-L5-VL2; VH1-L1-CL-L2-VL2-L3-CH1-L4-VL1-L5-VH2; VH1-L1-CL-L2-VH2-L3-CH1-L4-VL1-L5-VL2; VH1-L1-CH1-L2-VL1-L3-CL-L4-VL2-L5-VH2; VH1-L1-CH1-L2-VL1-L3-CL-L4-VH2-L5-VL2; VH1-L1-CH1-L2-VL2-L3-CL-L4-VL1-L5-VH2; VH1-L1-CH1-L2-VH2-L3-CL-L4-VL1-L5-VL2; VL2-L1-CL-L2-VL1-L3-CH1-L4-VH2-L5-VH1; VL2-L1-CL-L2-VH1-L3-CH1-L4-VH2-L5-VL1; VL2-L1-CL-L2-VH2-L3-CH1-L4-VL1-L5-VH1; VL2-L1-CL-L2-VH2-L3-CH1-L4-VH1-L5-VL1; VL2-L1-CH1-L2- VL1-L3-CL-L4-VH2-L5-VH1; VL2-L1-CH1-L2-VH1-L3-CL-L4-VH2-L5-VL1; VL2-L1-CH1-L2-VH2-L3-CL-L4-VL1-L5-VH1; VL2-L1-CH1-L2-VH2-L3-CL-L4-VH1-L5-VL1; VH2-L1-CL-L2-VL1-L3-CH1- L4-VL2-L5-VH1; VH2-L1-CL-L2-VH1-L3-CH1-L4-VL2-L5-VL1; VH2-L1-CL-L2-VL2-L3-CH1-L4-VL1-L5-VH1; VH2-L1-CL-L2-VL2-L3-CH1-L4-VH1-L5-VL1; VH2-L1-CH1-L2-VL1-L3-CL-L4-VL2-L5-VH1; VH2-L1-CH1-L2-VH1-L3-CL-L4-VL2-L5-VL1; VH2-L1-CH1-L2-VL2-L3-CL-L4-VL1-L5-VH1; or VH2-L1-CH1-L2-VL2-L3-CL-L4-VH1-L5-VL1; wherein the second polypeptide has a structure, from amino-terminus to carboxy-terminus, represented by: VL3-L6-CL-L7-VH3-L8-CH1-L9-VL4-L10-VH4; VL3-L6-CL-L7-VH3-L8-CH1-L9-VH4-L10-VL4; VL3-L6-CL-L7-VL4-L8-CH1-L9-VH3-L10-VH4; VL3-L6-CL-L7-VH4-L8-CH1-L9-VH3-L10-VL4; VL3-L6-CH1-L7-VH3-L8-CL-L9-VL4-L10-VH4; VL3-L6-CH1-L7-VH3-L8-CL-L9-VH4-L10-VL4; VL3-L6-CH1-L7-VL4-L8-CL-L9-VH3-L10-VH4; VL3-L6-CH1-L7-VH4-L8-CL-L9-VH3-L10-VL4; VH3-L6-CL-L7-VL3-L8-CH1-L9-VL4-L10-VH4; VH3-L6-CL-L7-VL3-L8-CH1-L9-VH4-L10-VL4; VH3-L6-CL-L7-VL4-L8-CH1-L9-VL3-L10-VH4; VH3-L6-CL-L7-VH4-L8-CH1-L9-VL3-L10-VL4; VH3-L6-CH1-L7-VL3-L8-CL-L9-VL4-L10-VH4; VH3-L6-CH1-L7-VL3-L8-CL-L9-VH4-L10-VL4; VH3-L6-CH1-L7-VL4-L8-CL-L9-VL3-L10-VH4; VH3-L6-CH1-L7-VH4-L8-CL-L9-VL3-L10-VL4; VL4-L6-CL-L7-VL3-L8-CH1-L9-VH4-L10-VH3; VL4-L6-CL-L7-VH3-L8-CH1-L9-VH4-L10-VL3; VL4-L6-CL-L7-VH4-L8-CH1-L9-VL3-L10-VH3; VL4-L6-CL-L7-VH4-L8-CH1-L9-VH3-L10-VL3; VL4-L6-CH1-L7-VL3-L8-CL-L9-VH4-L10-VH3; VL4-L6-CH1-L7-VH3-L8-CL-L9-VH4-L10-VL3; VL4-L6-CH1-L7-VH4-L8-CL-L9-VL3-L10-VH3; VL4-L6-CH1-L7-VH4-L8-CL-L9-VH3-L10-VL3; VH4-L6-CL-L7-VL3-L8-CH1-L9-VL4-L10-VH3; VH4-L6-CL-L7-VH3-L8-CH1-L9-VL4-L10-VL3; VH4-L6-CL-L7-VL4-L8-CH1-L9-VL3-L10-VH3; VH4-L6-CL-L7-VL4-L8-CH1-L9-VH3-L10-VL3; VH4-L6-CH1-L7-VL3-L8-CL-L9-VL4-L10-VH3; VH4-L6- CH1-L7-VH3-L8-CL-L9-VL4-L10-VL3; VH4-L6-CH1-L7-VL4-L8-CL-L9-VL3-L10-VH3; or VH4-L6-CH1- L7-VL4-L8-CL-L9-VH3-L10-VL3; and wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L10 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-VH1-L2-VL2-L3-CL-L4-VH2-L5-CH1; VL1-L1-VH1-L2-VL2-L3-CH1-L4-VH2-L5-CL; VL1-L1-VH1-L2-VH2-L3-CL-L4-VL2-L5-CH1; VL1-L1-VH1-L2-VH2-L3-CH1-L4-VL2-L5-CL; VL1-L1-VL2-L2-VH1-L3-CL-L4-VH2-L5-CH1; VL1-L1-VL2-L2-VH1-L3-CH1-L4-VH2-L5-CL; VL1-L1-VH2-L2-VH1-L3-CL-L4-VL2-L5-CH1; VL1-L1-VH2-L2-VH1-L3-CH1-L4-VL2-L5-CL; VH1-L1-VL1-L2-VL2-L3-CL-L4-VH2-L5-CH1; VH1-L1-VL1-L2-VL2-L3-CH1-L4-VH2-L5-CL; VH1-L1-VL1-L2-VH2-L3-CL-L4-VL2-L5-CH1; VH1-L1-VL1-L2-VH2-L3-CH1-L4-VL2-L5- CL; VH1-L1-VL2-L2-VL1-L3-CL-L4-VH2-L5-CH1; VH1-L1-VL2-L2-VL1-L3-CH1-L4-VH2-L5-CL; VH1-L1-VH2-L2-VL1-L3-CL-L4-VL2-L5-CH1; VH1-L1-VH2-L2-VL1-L3-CH1-L4-VL2-L5-CL; VL2-L1-VL1-L2-VH2-L3-CL-L4-VH1-L5-CH1; VL2-L1-VL1-L2-VH2-L3-CH1-L4-VH1-L5-CL; VL2-L1-VH1-L2-VH2-L3-CL-L4-VL1-L5-CH1; VL2-L1-VH1-L2-VH2-L3-CH1-L4-VL1-L5-CL; VL2-L1-VH2-L2-VL1-L3-CL-L4-VH1-L5-CH1; VL2-L1-VH2-L2-VL1-L3-CH1-L4-VH1-L5-CL; VL2-L1-VH2-L2-VH1-L3-CL-L4-VL1-L5-CH1; VL2-L1-VH2-L2-VH1-L3-CH1-L4-VL1-L5-CL; VH2-L1-VL1-L2-VL2-L3-CL-L4-VH1-L5-CH1; VH2-L1-VL1-L2-VL2-L3-CH1-L4-VH1-L5-CL; VH2-L1-VH1-L2-VL2-L3-CL-L4-VL1-L5-CH1; VH2-L1-VH1-L2-VL2-L3-CH1-L4-VL1-L5-CL; VH2-L1-VL2-L2-VL1-L3-CL-L4-VH1-L5-CH1; VH2-L1-VL2-L2-VL1-L3-CH1-L4-VH1-L5-CL; VH2-L1-VL2-L2-VH1-L3-CL-L4-VL1-L5-CH1; or VH2-L1-VL2-L2-VH1-L3-CH1-L4-VL1-L5-CL; wherein the second polypeptide has a structure, from amino-terminus to carboxy-terminus, represented by: VL3-L6-CL-L7-VH3-L8-CH1-L9-VL4-L10-VH4; VL3-L6-CL-L7-VH3-L8-CH1-L9-VH4-L10-VL4; VL3-L6-CL-L7-VL4-L8-CH1-L9-VH3-L10-VH4; VL3-L6-CL-L7-VH4-L8-CH1-L9-VH3-L10-VL4; VL3-L6-CH1-L7-VH3-L8-CL-L9-VL4-L10-VH4; VL3-L6-CH1-L7-VH3-L8-CL-L9-VH4-L10-VL4; VL3-L6-CH1-L7-VL4-L8-CL-L9-VH3-L10-VH4; VL3-L6-CH1-L7-VH4-L8-CL-L9-VH3-L10-VL4; VH3-L6-CL-L7-VL3-L8-CH1-L9-VL4-L10-VH4; VH3-L6-CL-L7-VL3-L8-CH1-L9-VH4-L10-VL4; VH3-L6-CL-L7-VL4-L8-CH1-L9-VL3-L10-VH4; VH3-L6-CL-L7-VH4-L8-CH1-L9-VL3-L10-VL4; VH3-L6-CH1-L7-VL3-L8-CL-L9-VL4-L10-VH4; VH3-L6-CH1-L7-VL3-L8-CL-L9-VH4-L10-VL4; VH3-L6-CH1-L7-VL4-L8-CL-L9-VL3-L10-VH4; VH3-L6-CH1-L7-VH4-L8-CL-L9-VL3-L10-VL4; VL4-L6-CL-L7-VL3-L8-CH1-L9-VH4-L10-VH3; VL4-L6-CL-L7-VH3-L8-CH1-L9-VH4-L10-VL3; VL4-L6-CL-L7-VH4-L8-CH1-L9-VL3-L10-VH3; VL4-L6-CL-L7-VH4-L8-CH1-L9-VH3-L10-VL3; VL4-L6-CH1-L7-VL3-L8-CL-L9-VH4-L10-VH3; VL4-L6-CH1-L7-VH3-L8-CL-L9-VH4-L10-VL3; VL4-L6-CH1-L7-VH4-L8-CL-L9-VL3-L10-VH3; VL4-L6-CH1-L7-VH4-L8-CL-L9-VH3-L10-VL3; VH4-L6-CL-L7-VL3-L8-CH1-L9-VL4-L10-VH3; VH4-L6-CL-L7-VH3-L8-CH1-L9-VL4-L10-VL3; VH4-L6-CL-L7-VL4-L8-CH1-L9-VL3-L10-VH3; VH4-L6-CL-L7-VL4-L8-CH1-L9-VH3-L10-VL3; VH4-L6-CH1-L7-VL3-L8-CL-L9-VL4-L10-VH3; VH4-L6-CH1-L7-VH3-L8-CL-L9-VL4-L10-VL3; VH4-L6-CH1-L7-VL4-L8-CL-L9-VL3-L10-VH3; or VH4-L6-CH1-L7-VL4-L8-CL-L9-VH3-L10-VL3; and wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L10 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VH1-L3-CH1-L4-VL2-L5-VH2; VL1-L1-CL-L2-VH1-L3-CH1-L4-VH2-L5-VL2; VL1-L1-CL-L2-VL2-L3-CH1-L4-VH1-L5-VH2; VL1-L1-CL-L2-VH2-L3-CH1-L4-VH1-L5-VL2; VL1-L1-CH1-L2-VH1-L3-CL-L4-VL2-L5-VH2; VL1-L1-CH1-L2-VH1-L3-CL-L4-VH2-L5-VL2; VL1-L1-CH1-L2-VL2-L3-CL-L4-VH1-L5-VH2; VL1-L1-CH1-L2-VH2-L3-CL-L4-VH1-L5-VL2; VH1-L1-CL-L2-VL1-L3-CH1-L4-VL2-L5-VH2; VH1-L1-CL-L2-VL1-L3-CH1-L4-VH2-L5-VL2; VH1-L1-CL-L2-VL2-L3-CH1-L4-VL1-L5-VH2; VH1-L1-CL-L2-VH2-L3-CH1-L4-VL1-L5-VL2; VH1-L1-CH1-L2-VL1-L3-CL-L4-VL2-L5-VH2; VH1-L1-CH1-L2-VL1-L3-CL-L4-VH2-L5-VL2; VH1-L1-CH1-L2-VL2-L3-CL-L4-VL1-L5-VH2; VH1-L1-CH1-L2-VH2-L3-CL-L4-VL1-L5-VL2; VL2-L1-CL-L2-VL1-L3-CH1-L4-VH2-L5-VH1; VL2-L1-CL-L2-VH1-L3-CH1-L4-VH2-L5-VL1; VL2-L1-CL-L2-VH2-L3-CH1-L4-VL1-L5-VH1; VL2-L1-CL-L2-VH2-L3-CH1-L4-VH1-L5-VL1; VL2-L1-CH1-L2- VL1-L3-CL-L4-VH2-L5-VH1; VL2-L1-CH1-L2-VH1-L3-CL-L4-VH2-L5-VL1; VL2-L1-CH1-L2-VH2-L3-CL-L4-VL1-L5-VH1; VL2-L1-CH1-L2-VH2-L3-CL-L4-VH1-L5-VL1; VH2-L1-CL-L2-VL1-L3-CH1- L4-VL2-L5-VH1; VH2-L1-CL-L2-VH1-L3-CH1-L4-VL2-L5-VL1; VH2-L1-CL-L2-VL2-L3-CH1-L4-VL1-L5-VH1; VH2-L1-CL-L2-VL2-L3-CH1-L4-VH1-L5-VL1; VH2-L1-CH1-L2-VL1-L3-CL-L4-VL2-L5-VH1; VH2-L1-CH1-L2-VH1-L3-CL-L4-VL2-L5-VL1; VH2-L1-CH1-L2-VL2-L3-CL-L4-VL1-L5-VH1; or VH2-L1-CH1-L2-VL2-L3-CL-L4-VH1-L5-VL1; wherein the second polypeptide has a structure, from amino-terminus to carboxy-terminus, represented by: VL3-L6-VH3-L7-VL4-L8-CL-L9-VH4-L10-CH1; VL3-L6-VH3-L7-VL4-L8-CH1-L9-VH4-L10-CL; VL3-L6-VH3-L7-VH4-L8-CL-L9-VL4-L10-CH1; VL3-L6-VH3-L7-VH4-L8-CH1-L9-VL4-L10-CL; VL3-L6-VL4-L7-VH3-L8-CL-L9-VH4-L10-CH1; VL3-L6-VL4-L7-VH3-L8-CH1-L9-VH4-L10-CL; VL3-L6-VH4-L7-VH3-L8-CL-L9-VL4-L10-CH1; VL3-L6-VH4-L7-VH3-L8-CH1-L9-VL4-L10-CL; VH3-L6-VL3-L7-VL4-L8-CL-L9-VH4-L10-CH1; VH3-L6-VL3-L7-VL4-L8-CH1-L9-VH4-L10-CL; VH3-L6-VL3-L7-VH4-L8-CL-L9-VL4-L10-CH1; VH3-L6-VL3-L7-VH4-L8-CH1-L9-VL4-L10-CL; VH3-L6-VL4-L7-VL3-L8-CL-L9-VH4-L10-CH1; VH3-L6-VL4-L7-VL3-L8-CH1-L9-VH4-L10-CL; VH3-L6-VH4-L7-VL3-L8-CL-L9-VL4-L10-CH1; VH3-L6-VH4-L7-VL3-L8-CH1-L9-VL4-L10-CL; VL4-L6-VL3-L7-VH4-L8-CL-L9-VH3-L10-CH1; VL4-L6-VL3-L7-VH4-L8-CH1-L9-VH3-L10-CL; VL4-L6-VH3-L7-VH4-L8-CL-L9-VL3-L10-CH1; VL4-L6-VH3-L7-VH4-L8-CH1-L9-VL3-L10-CL; VL4-L6-VH4-L7-VL3-L8-CL-L9-VH3-L10-CH1; VL4-L6-VH4-L7-VL3-L8-CH1-L9-VH3-L10-CL; VL4-L6-VH4-L7-VH3-L8-CL-L9-VL3-L10-CH1; VL4-L6-VH4-L7-VH3-L8-CH1-L9-VL3-L10-CL; VH4-L6-VL3-L7-VL4-L8-CL-L9-VH3-L10-CH1; VH4-L6-VL3-L7-VL4-L8-CH1-L9-VH3-L10-CL; VH4-L6-VH3-L7-VL4-L8-CL-L9-VL3-L10-CH1; VH4-L6-VH3-L7-VL4-L8-CH1-L9-VL3-L10-CL; VH4-L6-VL4-L7-VL3-L8-CL-L9-VH3-L10-CH1; VH4-L6-VL4-L7-VL3-L8-CH1-L9-VH3-L10-CL; VH4-L6-VL4-L7-VH3-L8-CL-L9-VL3-L10-CH1; or VH4-L6-VL4-L7-VH3-L8-CH1-L9-VL3-L10-CL; and wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L10 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VH1-L3-CH1; VL1-L1-CH1-L2-VH1-L3-CL; VH1-L1-CL-L2-VL1-L3-CH1; or VH1-L1-CH1-L2-VL1-L3-CL; wherein the second polypeptide has a structure, from amino-terminus to carboxy-terminus, represented by: VL2-L4-CL-L5-VL3-L6-VH3-L7-VH2-L8-CH1; VL2-L4-CL-L5-VH3-L6-VL3-L7-VH2-L8-CH1; VL2-L4-CH1-L5-VL3-L6-VH3-L7-VH2-L8- CL; VL2-L4-CH1-L5-VH3-L6-VL3-L7-VH2-L8-CL; VH2-L4-CL-L5-VL3-L6-VH3-L7-VL2-L8-CH1; VH2-L4-CL-L5-VH3-L6-VL3-L7-VL2-L8-CH1; VH2-L4-CH1-L5-VL3-L6-VH3-L7-VL2-L8-CL; VH2-L4-CH1-L5-VH3-L6-VL3-L7-VL2-L8-CL; VL3-L4-CL-L5-VL2-L6-VH2-L7-VH3-L8-CH1; VL3-L4-CL-L5-VH2-L6-VL2-L7-VH3-L8-CH1; VL3-L4-CH1-L5-VL2-L6-VH2-L7-VH3-L8-CL; VL3-L4-CH1-L5- VH2-L6-VL2-L7-VH3-L8-CL; VH3-L4-CL-L5-VL2-L6-VH2-L7-VL3-L8-CH1; VH3-L4-CL-L5-VH2-L6-VL2-L7-VL3-L8-CH1; VH3-L4-CH1-L5-VL2-L6-VH2-L7-VL3-L8-CL; or VH3-L4-CH1-L5-VH2-L6-VL2-L7-VL3-L8-CL; and wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L8 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VH1-L3-CH1; VL1-L1-CH1-L2-VH1-L3-CL; VH1-L1-CL-L2-VL1-L3-CH1; or VH1-L1-CH1-L2-VL1-L3-CL; wherein the second polypeptide has a structure, from amino-terminus to carboxy-terminus, represented by: VL2-L4-VH2-L5-VL3-L6-CL-L7-VH3-L8-CH1; VL2-L4-VH2-L5-VL3-L6-CH1-L7-VH3-L8-CL; VL2-L4-VH2-L5-VH3-L6-CL-L7-VL3-L8-CH1; VL2-L4-VH2-L5-VH3-L6-CH1-L7-VL3-L8-CL; VL2-L4-VL3-L5-VH2-L6-CL-L7-VH3-L8-CH1; VL2-L4-VL3-L5-VH2-L6-CH1-L7-VH3-L8-CL; VL2-L4-VH3-L5-VH2-L6-CL-L7-VL3-L8-CH1; VL2-L4-VH3-L5-VH2-L6-CH1-L7-VL3-L8-CL; VH2-L4-VL2-L5-VL3-L6-CL-L7-VH3-L8-CH1; VH2-L4-VL2-L5-VL3-L6-CH1-L7-VH3-L8-CL; VH2-L4-VL2-L5-VH3-L6-CL-L7-VL3-L8-CH1; VH2-L4-VL2-L5-VH3-L6-CH1-L7-VL3-L8-CL; VH2-L4-VL3-L5-VL2-L6-CL-L7-VH3-L8-CH1; VH2-L4-VL3-L5-VL2-L6-CH1-L7-VH3-L8-CL; VH2-L4-VH3-L5-VL2-L6-CL-L7-VL3-L8-CH1; VH2-L4-VH3-L5-VL2-L6-CH1-L7-VL3-L8-CL; VL3-L4-VL2-L5-VH3-L6-CL-L7-VH2-L8-CH1; VL3-L4-VL2-L5-VH3-L6-CH1-L7-VH2-L8-CL; VL3-L4-VH2-L5-VH3-L6-CL-L7-VL2-L8-CH1; VL3-L4-VH2-L5-VH3-L6-CH1-L7-VL2-L8-CL; VL3-L4-VH3-L5-VL2-L6-CL-L7-VH2-L8-CH1; VL3-L4-VH3-L5-VL2-L6-CH1-L7-VH2-L8-CL; VL3-L4-VH3-L5-VH2-L6-CL-L7-VL2-L8-CH1; VL3-L4-VH3-L5-VH2-L6-CH1-L7-VL2-L8- CL; VH3-L4-VL2-L5-VL3-L6-CL-L7-VH2-L8-CH1; VH3-L4-VL2-L5-VL3-L6-CH1-L7-VH2-L8-CL; VH3-L4-VH2-L5-VL3-L6-CL-L7-VL2-L8-CH1; VH3-L4-VH2-L5-VL3-L6-CH1-L7-VL2-L8-CL; VH3-L4-VL3-L5-VL2-L6-CL-L7-VH2-L8-CH1; VH3-L4-VL3-L5-VL2-L6-CH1-L7-VH2-L8-CL; VH3-L4-VL3-L5-VH2-L6-CL-L7-VL2-L8-CH1; or VH3-L4-VL3-L5-VH2-L6-CH1-L7-VL2-L8-CL; and wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L8 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-CL-L2-VH1-L3-CH1; VL1-L1-CH1-L2-VH1-L3-CL; VH1-L1-CL-L2-VL1-L3-CH1; or VH1-L1-CH1-L2-VL1-L3-CL; wherein the second polypeptide has a structure, from amino-terminus to carboxy-terminus, represented by: VL2-L4-CL-L5-VH2-L6-CH1-L7-VL3-L8-VH3; VL2-L4-CL-L5-VH2-L6-CH1-L7-VH3-L8-VL3; VL2-L4-CL-L5-VL3-L6-CH1-L7-VH2-L8-VH3; VL2-L4-CL-L5-VH3-L6-CH1-L7-VH2-L8-VL3; VL2-L4-CH1-L5-VH2-L6-CL-L7-VL3-L8-VH3; VL2-L4-CH1-L5-VH2-L6-CL-L7-VH3-L8-VL3; VL2-L4-CH1-L5-VL3-L6-CL-L7-VH2-L8-VH3; VL2-L4-CH1-L5-VH3-L6-CL-L7-VH2-L8-VL3; VH2-L4-CL-L5-VL2-L6-CH1-L7-VL3-L8-VH3; VH2-L4-CL-L5-VL2-L6-CH1-L7-VH3-L8-VL3; VH2-L4-CL-L5-VL3-L6-CH1-L7-VL2-L8-VH3; VH2-L4-CL-L5-VH3-L6-CH1-L7-VL2-L8-VL3; VH2-L4-CH1-L5-VL2-L6-CL-L7-VL3-L8-VH3; VH2-L4-CH1-L5- VL2-L6-CL-L7-VH3-L8-VL3; VH2-L4-CH1-L5-VL3-L6-CL-L7-VL2-L8-VH3; VH2-L4-CH1-L5-VH3-L6-CL-L7-VL2-L8-VL3; VL3-L4-CL-L5-VL2-L6-CH1-L7-VH3-L8-VH2; VL3-L4-CL-L5-VH2-L6-CH1- L7-VH3-L8-VL2; VL3-L4-CL-L5-VH3-L6-CH1-L7-VL2-L8-VH2; VL3-L4-CL-L5-VH3-L6-CH1-L7-VH2-L8-VL2; VL3-L4-CH1-L5-VL2-L6-CL-L7-VH3-L8-VH2; VL3-L4-CH1-L5-VH2-L6-CL-L7-VH3-L8-VL2; VL3-L4-CH1-L5-VH3-L6-CL-L7-VL2-L8-VH2; VL3-L4-CH1-L5-VH3-L6-CL-L7-VH2-L8-VL2; VH3-L4-CL-L5-VL2-L6-CH1-L7-VL3-L8-VH2; VH3-L4-CL-L5-VH2-L6-CH1-L7-VL3-L8-VL2; VH3-L4-CL-L5-VL3-L6-CH1-L7-VL2-L8-VH2; VH3-L4-CL-L5-VL3-L6-CH1-L7-VH2-L8-VL2; VH3-L4-CH1-L5-VL2-L6-CL-L7-VL3-L8-VH2; VH3-L4-CH1-L5-VH2-L6-CL-L7-VL3-L8-VL2; VH3-L4-CH1-L5-VL3-L6-CL-L7-VL2-L8-VH2; or VH3-L4-CH1-L5-VL3-L6-CL-L7-VH2-L8-VL2; and wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L8 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-VH1-L2-CL; or VH1-L3-VL1-L4-CL; wherein the second polypeptide has a structure, from amino-terminus to carboxy-terminus, represented by: VL2-L1-VH2-L2-CH1; or VH2-L3-VL2-L4-CH1; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L4 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-VL2-L2-CH1; or VL2-L1-VL1-L2-CH1; wherein the second polypeptide has a structure, from amino-terminus to carboxy-terminus, represented by: VH1-L3-VH2-L4-CL; or VH2-L3-VH1-L4-CL; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L4 are optional amino acid linkers.

Also provided herein is an antigen binding polypeptide complex comprising a first polypeptide and a second polypeptide, wherein the first polypeptide has a structure, from amino-terminus to carboxy-terminus, represented by: VL1-L1-VL2-L2-VL3-L3-CH1; VL1-L1-VL3-L2-VL2-L3-CH1; VL2-L1-VL1-L2-VL3-L3-CH1; VL2-L1-VL3-L2-VL1-L3-CH1; VL3-L1-VL1-L2-VL2-L3-CH1; or VL3-L1-VL2-L2-VL1-L3-CH1; wherein the second polypeptide has a structure, from amino-terminus to carboxy-terminus, represented by: VH1-L4-VH2-L5-VH3-L6-CL; VH1-L4-VH3-L5-VH2-L6-CL; VH2-L4-VH1-L5-VH3-L6-CL; VH2-L4-VH3-L5-VH1-L6-CL; VH3-L4-VH1-L5-VH2-L6-CL; or VH3-L4-VH2-L5-VH1-L6- CL; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to a (i) a tumor-associated antigen (TAA), (ii) an infectious disease antigen that is not a sarbecovirus antigen, or (iii) an other-pathology antigen; CL is an immunoglobulin light chain constant region; CH1 is an immunoglobulin heavy chain constant region 1; and L1-L6 are optional amino acid linkers.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgen receptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSF1R, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-related antigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1α, IL-1β, IL2, IL-20, IL22, IL23, IL-23A, IL25, IL2Rbeta, IL-3 receptor, IL-31 receptor A, IL33, IL35, IL4, IL4Ra, IL5, ILSR, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxylesterase, ITGB+, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL, Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRP1 glycoprotein 75, UPK2, VAP-1, VEGF, VEGFA, VEGFR-1, VEGFR2, vimentin, VISTA, Vstm3, WTI, WUCAM, and XAGE1.

In some aspects, and in any one of the formulas depicted herein, any one or more of (i) VL1 and VH1, (ii) VL2 and VH2, (iii) VL3 and VH3, and (iv) VL4 and VH4, comprise a light chain complementarity determining region 1 (LCDR1), LCDR2, and/or LCDR3, and a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140), rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, cramkafusp alfa, ripertamab, zuberitamab, glofitamab, nofetumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirinc, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, sclicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmolcukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, cpitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650), tralokinumab, anrukinzumab, brodalumab, ixckizumab, netakimab, perakizumab, secukinumab, vunakizumab, afasevikumab, afasevikumab, bimckizumab, bimckizumab, remtolumab, bermekimab, lutikizumab, lutikizumab, gevokizumab, cergutuzumab amunalcukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, clsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, cfalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, clotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, ctigilimab, tiragotumab, placulumab, prezalumab, infliximab, nerelimomab, ozoralizumab, adalimumab, afclimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, and in any one of the formulas depicted herein, any one or more of (i) VL1 and VH1, (ii) VL2 and VH2, (iii) VL3 and VH3, and (iv) VL4 and VH4, comprise a VL and a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140), rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, cramkafusp alfa, ripertamab, zuberitamab, glofitamab, nofetumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, cpcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adccatumumab, adecatumumab, oportuzumab monatox, solitomab, tucotuzumab celmolcukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650), tralokinumab, anrukinzumab, brodalumab, ixckizumab, netakimab, perakizumab, sccukinumab, vunakizumab, afasevikumab, afasevikumab, bimekizumab, bimckizumab, remtolumab, bermekimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, and in any one of the formulas depicted herein, (a) any one or more of VL1, VL2, VL3, and VL4 comprises: (i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a LCDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777, or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976; and (b) any one or more of VH1, VH2, VH3, VH4 comprises: (i) a HCDR1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a HCDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, and in any one of the formulas depicted herein, (a) any one or more of VL1, VL2, VL3, VL4 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788; and (b) any one or more of VH1, VH2, VH3, VH4 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is: (i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, HON2). Yamagata virus antigen. Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-HON2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase): parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein. RSV G glycoprotein. F protein of respiratory syncytial virus. RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55). HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50) (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1. HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or (ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin). Corynebacterium ulcerans. Corynebacterium pseudotuberculosis). Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium. Mycoplasma hyopneumoniae. Mycoplasma pneumonia) antigen, a Staphylococcus (e.g., Staphylococcus aureus, coagulase-negative staphylococci (CoNS), Staphylococcus aureus alpha toxin. Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine. Hsp90. CCR5, lipoteichoic acid, clumping factor A).

In some aspects, and in any one of the formulas depicted herein, any one or more of (i) VL1 and VH1, (ii) VL2 and VH2, (iii) VL3 and VH3, and (iv) VL4 and VH4, comprise a light complementarity determining region 1 (LCDR1), LCDR2, and/or LCDR3, and a heavy complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab. REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, and in any one of the formulas depicted herein, any one or more of (i) VL1 and VH1, (ii) VL2 and VH2, (iii) VL3 and VH3, and (iv) VL4 and VH4, comprise a VL and a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, and in any one of the formulas depicted herein, (a) any one or more of VL1, VL2, VL3, and VL4 comprises: (i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a LCDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002, or at least 65%, at least 70%, at least 75%, at least 80%, at least 85%, at least 90%, at least 95%, at least 99%, or 100% identical to any one of amino acid sequences YTS, DTS, GAS, and AAS; and (iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003; and (b) any one or more of VH1, VH2, VH3, and VH4 comprises: (i) a HCDR1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a HCDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, and in any one of the formulas depicted herein, (a) any one or more of VL1, VL2, VL3, and VL4 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004; and (b) any one or more of VH1, VH2, VH3, and VH4 comprises an amino acid sequence that is at least 65%, at least 70%, at least 75%, at least 80%, at least 85%, at least 90%, at least 95%, at least 99%, or 100% identical to any one of SEQ ID NOS: 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/B receptor. Rhesus factor, nerve growth factor (NGF), DPP4, fibrin II beta chain. ACVR2B, scrum amyloid A protein SOST, FGF 23, VWF, tissue factor pathway inhibitor (TFPI), 1-40) and 1-42, selectin P, glucagon receptor (GCGR), amyloid P component, GDF-8, CXCL10) IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region. MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, and in any one of the formulas depicted herein, any one or more of (i) VL1 and VH1, (ii) VL2 and VH2, (iii) VL3 and VH3, and (iv) VL4 and VH4, comprise a light complementarity determining region 1 (LCDR1), LCDR2, and/or LCDR3, and a heavy complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, and in any one of the formulas depicted herein, any one or more of (i) VL1 and VH1, (ii) VL2 and VH2, (iii) VL3 and VH3, and (iv) VL4 and VH4, comprise the VL and the VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, and in any one of the formulas depicted herein, (a) any one or more of VL1, VL2, VL3, and VL4 comprises: (i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a LCDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970; and (b) any one or more of VH1, VH2, VH3, and VH4 comprises: (i) a HCDR1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a HCDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, and in any one of the formulas depicted herein, (a) any one or more of VL1, VL2, and VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971; and (b) any one or more of VH1, VH2, and VH3 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

Also provided herein is an antigen binding polypeptide complex comprising a first polypeptide and a second polypeptide, wherein the first polypeptide has a structure, from amino-terminus to carboxy-terminus, represented by: VL1-L1-VL2-L2-VH2-L3-VH1-L4-CL-L5-CH1; VL1-L1-VL2-L2-VH2-L3-VH1-L4-CH1-L5-CL; VL1-L1-VH2-L2-VL2-L3-VH1-L4-CL-L5-CH1; VL1-L1-VH2-L2-VL2-L3-VH1-L4-CH1-L5-CL; VH1-L1-VL2-L2-VH2-L3-VL1-L4-CL-L5-CH1; VH1-L1-VL2-L2-VH2-L3-VL1-L4-CH1-L5-CL; VH1-L1-VH2-L2-VL2-L3-VL1-L4-CL-L5-CH1; VH1-L1-VH2-L2-VL2-L3-VL1-L4-CH1-L5-CL; VL2-L1-VL1-L2-VH1-L3-VH2-L4-CL-L5-CH1; VL2-L1-VL1-L2-VH1-L3-VH2-L4-CH1-L5-CL; VL2-L1-VH1-L2-VL1-L3-VH2-L4-CL-L5-CH1; VL2-L1-VH1-L2-VL1-L3-VH2-L4-CH1-L5- CL; VH2-L1-VL1-L2-VH1-L3-VL2-L4-CL-L5-CH1; VH2-L1-VL1-L2-VH1-L3-VL2-L4-CH1-L5-CL; VH2-L1-VH1-L2-VL1-L3-VL2-L4-CL-L5-CH1; or VH2-L1-VH1-L2-VL1-L3-VL2-L4-CH1-L5-CL; wherein the second polypeptide has a structure, from amino-terminus to carboxy-terminus, represented by: VL3-L6-VL4-L7-VH4-L8-VH3-L9-CL-L10-CH1; VL3-L6-VL4-L7-VH4-L8-VH3-L9-CH1-L10-CL; VL3-L6-VH4-L7-VL4-L8-VH3-L9-CL-L10-CH1; VL3-L6-VH4-L7-VL4-L8-VH3-L9-CH1-L10-CL; VH3-L6-VL4-L7-VH4-L8-VL3-L9-CL-L10-CH1; VH3-L6-VL4-L7-VH4-L8-VL3-L9-CH1-L10-CL; VH3-L6-VH4-L7-VL4-L8-VL3-L9-CL-L10-CH1; VH3-L6-VH4-L7-VL4-L8-VL3-L9-CH1-L10-CL; VL4-L6-VL3-L7-VH3-L8-VH4-L9-CL-L10-CH1; VL4-L6-VL3-L7-VH3-L8-VH4-L9-CH1-L10-CL; VL4-L6-VH3-L7-VL3-L8-VH4-L9-CL-L10-CH1; VL4-L6-VH3-L7-VL3-L8-VH4-L9-CH1-L10-CL; VH4-L6-VL3-L7-VH3-L8-VL4-L9-CL-L10-CH1; VH4-L6-VL3-L7-VH3-L8-VL4-L9-CH1-L10-CL; VH4-L6-VH3-L7-VL3-L8-VL4-L9-CL-L10-CH1; or VH4-L6-VH3-L7-VL3-L8-VL4-L9-CH1-L10-CL; and wherein: (a) VL1 is a first immunoglobulin light chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786, a CDR2 having an amino acid sequence at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS or an amino acid sequence at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777, and a CDR3 having an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976; VL2 is a second immunoglobulin light chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786, a CDR2 having an amino acid sequence at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS or an amino acid sequence at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777, and a CDR3 having an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976; VL3 is a third immunoglobulin light chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786, a CDR2 having an amino acid sequence at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS or an amino acid sequence at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777, and a CDR3 having an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976; VL4 is a fourth immunoglobulin light chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786, a CDR2 having an amino acid sequence at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS or an amino acid sequence at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777, and a CDR3 having an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976; VH1 is a first immunoglobulin heavy chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789, a CDR2 having an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790, and a CDR3 having an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; VH2 is a second immunoglobulin heavy chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789, a CDR2 having an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790, and a CDR3 having an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; VH3 is a third immunoglobulin heavy chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789, a CDR2 having an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790, and a CDR3 having an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and VH4 is a fourth immunoglobulin heavy chain variable region that specifically binds to a tumor-associated antigen (TAA), comprising a CDR1 having an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789, a CDR2 having an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790, and a CDR3 having an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; (b) VL1 is a first immunoglobulin light chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001, a CDR2 having an amino acid sequence at least 65%, at least 70%, at least 75%, at least 80%, at least 85%, at least 90%, at least 95%, at least 99%, or 100% identical to any one of amino acid sequences YTS, DTS, GAS, and AAS or an amino acid sequence at least 65%, at least 70%, at least 75%, at least 80%, at least 85%, at least 90%, at least 95%, at least 99%, or 100% identical to any one of SEQ ID NOs: 1978, 1986, 1994, and 2002, and a CDR3 having an amino acid sequence at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003; VL2 is a second immunoglobulin light chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001, a CDR2 having an amino acid sequence at least 65%, at least 70%, at least 75%, at least 80%, at least 85%, at least 90%, at least 95%, at least 99%, or 100% identical to any one of amino acid sequences YTS, DTS, GAS, and AAS or an amino acid sequence at least 65%, at least 70%, at least 75%, at least 80%, at least 85%, at least 90%, at least 95%, at least 99%, or 100% identical to any one of SEQ ID NOs: 1978, 1986, 1994, and 2002, and a CDR3 having an amino acid sequence at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003; VL3 is a third immunoglobulin light chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001, a CDR2 having an amino acid sequence at least 65%, at least 70%, at least 75%, at least 80%, at least 85%, at least 90%, at least 95%, at least 99%, or 100% identical to any one of amino acid sequences YTS, DTS, GAS, and AAS or an amino acid sequence at least 65%, at least 70%, at least 75%, at least 80%, at least 85%, at least 90%, at least 95%, at least 99%, or 100% identical to any one of SEQ ID NOs: 1978, 1986, 1994, and 2002, and a CDR3 having an amino acid sequence at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003; VL4 is a fourth immunoglobulin light chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001, a CDR2 having an amino acid sequence at least 65%, at least 70%, at least 75%, at least 80%, at least 85%, at least 90%, at least 95%, at least 99%, or 100% identical to any one of amino acid sequences YTS, DTS, GAS, and AAS or an amino acid sequence at least 65%, at least 70%, at least 75%, at least 80%, at least 85%, at least 90%, at least 95%, at least 99%, or 100% identical to any one of SEQ ID NOs: 1978, 1986, 1994, and 2002, and a CDR3 having an amino acid sequence at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003; VH1 is a first immunoglobulin heavy chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005, a CDR2 having an amino acid sequence at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006, and a CDR3 having an amino acid sequence at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; VH2 is a second immunoglobulin heavy chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005, a CDR2 having an amino acid sequence at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006, and a CDR3 having an amino acid sequence at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; VH3 is a third immunoglobulin heavy chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005, a CDR2 having an amino acid sequence at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006, and a CDR3 having an amino acid sequence at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and VH4 is a fourth immunoglobulin heavy chain variable region that specifically binds to an infectious disease antigen that is not a sarbecovirus antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005, a CDR2 having an amino acid sequence at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006, and a CDR3 having an amino acid sequence at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or (c) VL1 is a first immunoglobulin light chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963, a CDR2 having an amino acid sequence at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS, and a CDR3 having an amino acid sequence at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970; VL2 is a second immunoglobulin light chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963, a CDR2 having an amino acid sequence at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS, and a CDR3 having an amino acid sequence at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970; VL3 is a third immunoglobulin light chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963, a CDR2 having an amino acid sequence at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS, and a CDR3 having an amino acid sequence at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970; VL4 is a fourth immunoglobulin light chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963, a CDR2 having an amino acid sequence at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS, and a CDR3 having an amino acid sequence at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970; VH1 is a first immunoglobulin heavy chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966, a CDR2 having an amino acid sequence at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972, and a CDR3 having an amino acid sequence at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; VH2 is a second immunoglobulin heavy chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966, a CDR2 having an amino acid sequence at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972, and a CDR3 having an amino acid sequence at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; VH3 is a third immunoglobulin heavy chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966, a CDR2 having an amino acid sequence at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972, and a CDR3 having an amino acid sequence at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and VH4 is a fourth immunoglobulin heavy chain variable region that specifically binds to an other-pathology antigen, comprising a CDR1 having an amino acid sequence at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966, a CDR2 having an amino acid sequence at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972, and a CDR3 having an amino acid sequence at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and wherein: CL is an immunoglobulin light chain constant region; CH1 is an immunoglobulin heavy chain constant region 1; and L1-L10 are optional amino acid linkers.

In some aspects, and in any one of the formulas depicted herein, (a) VL1 comprises an amino acid sequence that is at least 65%, at least 70%, at least 75%, at least 80%, at least 85%, at least 90%, at least 95%, at least 99%, or 100% identical to SEQ ID NO: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788; VL2 comprises an amino acid sequence that is at least 65%, at least 70%, at least 75%, at least 80%, at least 85%, at least 90%, at least 95%, at least 99%, or 100% identical to SEQ ID NO: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788; VL3 comprises an amino acid sequence that is at least 65%, at least 70%, at least 75%, at least 80%, at least 85%, at least 90%, at least 95%, at least 99%, or 100% identical to SEQ ID NO: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788; VL4 comprises an amino acid sequence that is at least 65%, at least 70%, at least 75%, at least 80%, at least 85%, at least 90%, at least 95%, at least 99%, or 100% identical to SEQ ID NO: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788; VH1 comprises an amino acid sequence that is at least 65%, at least 70%, at least 75%, at least 80%, at least 85%, at least 90%, at least 95%, at least 99%, or 100% identical to SEQ ID NO: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; VH2 comprises an amino acid sequence that is at least 65%, at least 70%, at least 75%, at least 80%, at least 85%, at least 90%, at least 95%, at least 99%, or 100% identical to SEQ ID NO: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; VH3 comprises an amino acid sequence that is at least 65%, at least 70%, at least 75%, at least 80%, at least 85%, at least 90%, at least 95%, at least 99%, or 100% identical to SEQ ID NO: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and VH4 comprises an amino acid sequence that is at least 65%, at least 70%, at least 75%, at least 80%, at least 85%, at least 90%, at least 95%, at least 99%, or 100% identical to SEQ ID NO: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; (b) VL1 comprises an amino acid sequence that is at least 65%, at least 70%, at least 75%, at least 80%, at least 85%, at least 90%, at least 95%, at least 99%, or 100% identical to SEQ ID NO: 1794, 1800, 1806, 1812, 1819, 1826, 1980, 1988, 1996, and 2004; VL2 comprises an amino acid sequence that is at least 65%, at least 70%, at least 75%, at least 80%, at least 85%, at least 90%, at least 95%, at least 99%, or 100% identical to SEQ ID NO: 1794, 1800, 1806, 1812, 1819, 1826, 1980, 1988, 1996, and 2004; VL3 comprises an amino acid sequence that is at least 65%, at least 70%, at least 75%, at least 80%, at least 85%, at least 90%, at least 95%, at least 99%, or 100% identical to SEQ ID NO: 1794, 1800, 1806, 1812, 1819, 1826, 1980, 1988, 1996, and 2004; VL4 comprises an amino acid sequence that is at least 65%, at least 70%, at least 75%, at least 80%, at least 85%, at least 90%, at least 95%, at least 99%, or 100% identical to SEQ ID NO: 1794, 1800, 1806, 1812, 1819, 1826, 1980, 1988, 1996, and 2004; VH1 comprises an amino acid sequence that is at least 65%, at least 70%, at least 75%, at least 80%, at least 85%, at least 90%, at least 95%, at least 99%, or 100% identical to SEQ ID NO: 1797, 1804, 1809, 1816, 1823, 1830, 1984, 1992, 2000, and 2008; VH2 comprises an amino acid sequence that is at least 65%, at least 70%, at least 75%, at least 80%, at least 85%, at least 90%, at least 95%, at least 99%, or 100% identical to SEQ ID NO: 1797, 1804, 1809, 1816, 1823, 1830, 1984, 1992, 2000, and 2008; VH3 comprises an amino acid sequence that is at least 65%, at least 70%, at least 75%, at least 80%, at least 85%, at least 90%, at least 95%, at least 99%, or 100% identical to SEQ ID NO: 1797, 1804, 1809, 1816, 1823, 1830, 1984, 1992, 2000, and 2008; and VH4 comprises an amino acid sequence that is at least 65%, at least 70%, at least 75%, at least 80%, at least 85%, at least 90%, at least 95%, at least 99%, or 100% identical to SEQ ID NO: 1797, 1804, 1809, 1816, 1823, 1830, 1984, 1992, 2000, and 2008; or (c) VL1 comprises an amino acid sequence that is at least 65%, at least 70%, at least 75%, at least 80%, at least 85%, at least 90%, at least 95%, at least 99%, or 100% identical to SEQ ID NO: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971; VL2 comprises an amino acid sequence that is at least 65%, at least 70%, at least 75%, at least 80%, at least 85%, at least 90%, at least 95%, at least 99%, or 100% identical to SEQ ID NO: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971; VL3 comprises an amino acid sequence that is at least 65%, at least 70%, at least 75%, at least 80%, at least 85%, at least 90%, at least 95%, at least 99%, or 100% identical to SEQ ID NO: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971; VL4 comprises an amino acid sequence that is at least 65%, at least 70%, at least 75%, at least 80%, at least 85%, at least 90%, at least 95%, at least 99%, or 100% identical to SEQ ID NO: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971; VH1 comprises an amino acid sequence that is at least 65%, at least 70%, at least 75%, at least 80%, at least 85%, at least 90%, at least 95%, at least 99%, or 100% identical to SEQ ID NO: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; VH2 comprises an amino acid sequence that is at least 65%, at least 70%, at least 75%, at least 80%, at least 85%, at least 90%, at least 95%, at least 99%, or 100% identical to SEQ ID NO: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; VH3 comprises an amino acid sequence that is at least 65%, at least 70%, at least 75%, at least 80%, at least 85%, at least 90%, at least 95%, at least 99%, or 100% identical to SEQ ID NO: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and VH4 comprises an amino acid sequence that is at least 65%, at least 70%, at least 75%, at least 80%, at least 85%, at least 90%, at least 95%, at least 99%, or 100% identical to SEQ ID NO: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, and in any one of the formulas depicted herein, the one or more CL comprise an amino acid sequence that is at least 65%, at least 70%, at least 75%, at least 80%, at least 85%, at least 90%, at least 95%, at least 99%, or 100% identical to any one of SEQ ID NOs: 374, 637, or 1092-1095. In some aspects, and in any one of the formulas depicted herein, the one or more CH1 comprise an amino acid sequence that is at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptide complex further comprises one or more immunoglobulin heavy chain constant region 2 (CH2), and wherein the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078.

In some aspects, the antigen binding polypeptide complex further comprises one or more immunoglobulin heavy chain constant region 3 (CH3), and wherein the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085.

In some aspects, the first polypeptide and the second polypeptide each further comprise an Fc region at the carboxy-terminus. In some aspects, the Fc region of the first polypeptide and the second polypeptide each comprise an immunoglobulin heavy chain constant region 2 (CH2) and/or an immunoglobulin heavy chain constant region 3 (CH3). In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085.

In some aspects, the Fc region of the first polypeptide and the second polypeptide each further comprise an immunoglobulin hinge. In some aspects, the immunoglobulin hinge comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074.

In some aspects, the Fc region of the first polypeptide and the second polypeptide comprise one or more half-life extension amino acid substitutions, one or more knob-into-hole modification, one or more Fc effector function knockout mutation, or any combination thereof. In some aspects, the Fc region is an IgG Fc region. In some aspects, the Fc region is an IgG1 or an IgG4 Fc region. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise amino acid substitutions: (i) L234A and L235A (LA); (ii) M428L and N434S (LS); (iii) L234F and L235E; (iv) L234F, L235E, and P331S (TM); and/or (v) M252Y, S254T, and T256E (YTE); or equivalent thereof, based on the EU numbering scheme. In some aspects, the one or more the knob-into-hole modification comprises: (i) knob substitutions of S354C and T366W; (ii) hole substitutions of Y349C, T366S, L368A and Y407V; or (iii) a combination thereof; or equivalent thereof, based on the EU numbering scheme. In some aspects, the one or more Fc effector function knockout mutation is L234A, L235A, P239A, or equivalent thereof, or a combination thereof, based on the EU numbering scheme. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, and in any one of the formulas shown herein, any one of the optional amino acid linkers each independently have a length of from 0 amino acids to about 50 amino acids. In some aspects, and in any one of the formulas shown herein, any one of the optional linkers each independently have a length of from about 5 amino acids to about 40 amino acids. In some aspects, and in any one of the formulas shown herein, one or more of the optional linkers each independently are non-immunogenic. In some aspects, and in any one of the formulas shown herein, one or more of the optional linkers each independently do not contain a consensus T cell epitope. In some aspects, and in any one of the formulas shown herein, one or more of the optional linkers each independently comprises an amino acid sequence of G or A; or an amino acid sequence at least 65%, at least 70%, at least 75%, at least 80%, at least 85%, at least 90%, at least 95%, at least 99%, or 100% identical to an amino acid sequence of GSS or ASG; or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, and in any one of the formulas shown herein, one or more of the optional linkers each independently comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, and in any one of the formulas shown herein, one or more of the VH has a mutation at an N-glycosylation site. In some aspects, and in any one of the formulas shown herein, the mutation is at amino acid N62 of the VH region or equivalent thereof. In some aspects, and in any one of the formulas shown herein, the N62 mutation is N62Q or N62S, or equivalent thereof.

In some aspects, the antigen binding polypeptide complex disclosed herein has an isoelectric point (pI) of from about 7 to about 9.

In some aspects, the antigen binding polypeptide or antigen binding polypeptide complex disclosed herein comprises a detectable label. In some aspects, the detectable label is a radioactive label, chemiluminescent label, fluorescent label, enzyme, peptide tag, or a combination thereof. In some aspects, the peptide tag is a poly histidine tag consisting of from about four to about 10 histidine residues. In some aspects, the poly histidine tag consists of about eight histidine residues.

In some aspects, the antigen binding polypeptide or antigen binding polypeptide complex disclosed herein is conjugated to an agent as an antibody-drug conjugate (ADC). In some aspects, the agent is a cytotoxic agent, immunomodulating agent, imaging agent, or therapeutic protein, or a combination thereof.

In some aspects, the antigen binding polypeptide or antigen binding polypeptide complex disclosed herein specifically binds to the (i) tumor-associated antigen (TAA), (ii) infectious disease antigen that is not a sarbecovirus antigen, or (iii) other-pathology antigen with an equilibrium dissociation constant (K_{D}) of from about 10 μM to about 1 pM.

In some aspects, the antigen binding polypeptide complex disclosed herein is bispecific, trispecific or tetraspecific and has greater binding affinity to the (i) tumor-associated antigen (TAA), (ii) infectious disease antigen that is not a sarbecovirus antigen, or (iii) other-pathology antigen than a monospecific antigen binding polypeptide complex that specifically binds to one of the same antigens as the bispecific, trispecific or tetraspecific antigen binding polypeptide complex.

In some aspects, the antigen binding polypeptide complex disclosed herein is bispecific, trispecific or tetraspecific and has greater binding affinity to the (i) tumor-associated antigen (TAA), (ii) infectious disease antigen that is not a sarbecovirus antigen, or (iii) other-pathology antigen than a mixture of monospecific antigen binding polypeptide complexes that specifically bind to the same antigens as the bispecific, trispecific or tetraspecific antigen binding polypeptide complex.

Also provided herein is an antibody or antigen binding fragment thereof comprising an antigen binding polypeptide or antigen binding polypeptide complex disclosed herein.

In some aspects, the antibody or antigen binding fragment thereof is IgG, IgM, IgE, IgA or IgD. In some aspects, the IgG is IgG1, IgG2, IgG3, or IgG4.

In some aspects, the antigen binding fragment is a Fab, scFab, Fab′, F(ab′)₂. Fv or scFv.

In some aspects, the antibody or antigen binding fragment thereof is human or humanized.

Also provided herein is a polynucleotide encoding the antigen binding polypeptide or antigen binding polypeptide complex, or the antibody or antigen binding fragment thereof disclosed herein.

Also provided herein is a vector comprising the polynucleotide disclosed herein.

Also provided herein is a host cell comprising the vector disclosed herein.

Also provided herein is an mRNA encoding the antigen binding polypeptide or antigen binding polypeptide complex, or the antibody or antigen binding fragment thereof disclosed herein. In some aspects, the mRNA comprises a 5′-cap and/or a poly (A) tail.

Also provided herein is a lipid nanoparticle (LNP) comprising the mRNA disclosed herein.

Also provided herein is a chimeric antigen receptor (CAR) comprising the antigen binding polypeptide or antigen binding polypeptide complex disclosed herein.

Also provided herein is an immune cell comprising the CAR disclosed herein.

Also provided herein is a pharmaceutical composition comprising the antigen binding polypeptide or antigen binding polypeptide complex, the antibody or antigen binding fragment thereof, the polynucleotide, the vector, the host cell, the mRNA, the LNP, the CAR, or the immune cell disclosed herein.

Also provided herein is a pharmaceutical composition comprising (i) the antigen binding polypeptide or antigen binding polypeptide complex, the antibody or antigen binding fragment thereof, the polynucleotide, the vector, the host cell, the mRNA, the LNP, the CAR, or the immune cell disclosed herein, and (ii) a pharmaceutically acceptable carrier.

Also provided herein is a pharmaceutical composition comprising (i) the antigen binding polypeptide or antigen binding polypeptide complex, the antibody or antigen binding fragment thereof, the polynucleotide, the vector, the host cell, the mRNA, the LNP, the CAR, or the immune cell disclosed herein, and (ii) an additional pharmaceutical agent.

Also provided herein is a kit comprising (i) the antigen binding polypeptide or antigen binding polypeptide complex, the antibody or antigen binding fragment thereof, the polynucleotide, the vector, the host cell, the mRNA, the LNP, the CAR, the immune cell, or the pharmaceutical composition disclosed herein, and (ii) instructions for use.

Also provided herein is a method of preventing or treating (i) a cancer or a tumor, (ii) an infectious disease that is not a sarbecovirus infectious disease, or (iii) a pathology other than a cancer or a tumor or an infectious disease that is not a sarbecovirus infectious disease, comprising administering to the subject a therapeutically effective amount of the antigen binding polypeptide or antigen binding polypeptide complex, the antibody or antigen binding fragment thereof, the polynucleotide, the vector, the host cell, the mRNA, the LNP, the CAR, the immune cell, or the pharmaceutical composition disclosed herein.

Also provided herein is a method of diagnosing a subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptide or antigen binding polypeptide complex or the antibody or antigen binding fragment thereof disclosed herein; (b) detecting the presence or absence of a complex which contains the polypeptide or a fragment thereof, polypeptide complex or a fragment thereof, or antibody or a fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is detected.

Also provided herein is a method of diagnosing a subject as not having or as not being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptide or antigen binding polypeptide complex or the antibody or antigen binding fragment thereof disclosed herein; (b) detecting the presence or absence of a complex which contains the polypeptide or a fragment thereof, polypeptide complex or a fragment thereof, or antibody or a fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as not having or as not being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is not detected. In some aspects, the sample is a nasal swab, tissue sample, saliva, plasma or blood. In some aspects, detecting the presence or absence of the complex comprises an enzyme linked immunosorbent assay (ELISA), immunospot assay, lateral flow assay, flow cytometry, immunohistochemistry or western blot.

Also provided herein is an antibody or an antigen binding fragment thereof, wherein the antibody or antigen binding fragment thereof binds to (i) a tumor-associated antigen (TAA), (ii) an infectious disease antigen that is not a sarbecovirus antigen, or (iii) an other-pathology antigen, and wherein the antibody or antigen binding fragment thereof comprises: (a) one or more immunoglobulin light chain constant domain (CL) and (b) one or more immunoglobulin heavy chain 1 constant domain (CH1); and wherein the antibody or antigen binding fragment thereof (i) has an isoelectric point (pI), and after administered to a subject has (ii) a higher peak serum levels, and (iii) a reduced clearance levels, as compared to a same antibody or an antigen binding fragment thereof not comprising one or more immunoglobulin light chain constant domain (CL) and (b) one or more immunoglobulin heavy chain 1 constant domain (CH1).

In some aspects, the antibody or antigen binding fragment thereof is IgG, IgM, IgE, IgA or IgD. In some aspects, the antibody or antigen binding fragment thereof is IgG1, IgG2, IgG3, or IgG4. In some aspects, the antibody or antigen binding fragment thereof is a Fab, scFab, Fab′, F(ab′)₂. Fv or scFv. In some aspects, the antibody or antigen binding fragment thereof is human or humanized.

In some aspects, the antibody or antigen binding fragment thereof has an isoelectric point (pI) of from about 7 to about 9.

In some aspects, the antibody or antigen binding fragment thereof binds to the (i) tumor-associated antigen (TAA), (ii) infectious disease antigen that is not a sarbecovirus antigen, or (iii) other-pathology antigen with an equilibrium dissociation constant (K_{D}) of from about 10 μM to about 1 pM.

In some aspects, the antibody or antigen binding fragment thereof is bispecific, trispecific or tetraspecific and has greater binding affinity to the (i) tumor-associated antigen (TAA), (ii) infectious disease antigen that is not a sarbecovirus antigen, or (iii) other-pathology antigen than a monospecific antibody or an antigen binding fragment thereof that specifically binds to one of the same antigens as the bispecific, trispecific or tetraspecific antibody or antigen binding fragment thereof.

In some aspects, the antibody or an antigen binding fragment thereof is bispecific, trispecific or tetraspecific and has greater binding affinity to the (i) tumor-associated antigen (TAA), (ii) infectious disease antigen that is not a sarbecovirus antigen, or (iii) other-pathology antigen than a mixture of monospecific antibodies or antigen binding fragments thereof that specifically binds to one of the same antigens as the bispecific, trispecific or tetraspecific antibody or antigen binding fragment thereof.

In some aspects, the antibody or antigen binding fragment thereof comprises one or more variable heavy chain regions (VH) and wherein at least one of the one or more VH has a mutation at an N-glycosylation site. In some aspects, the mutation is at amino acid N62 of the VH region or equivalent thereof. In some aspects, the N62 mutation is N62Q or N62S, or equivalent thereof.

In some aspects, the antibody or antigen binding fragment thereof comprises at least one variable heavy chain region (VH) and/or at least one variable light chain region (VL).

In some aspects, (a) the at least one VL comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002, or at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003; and/or (b) the at least one VH comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007.

In some aspects, (a) the at least one VL comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; and/or (b) the at least one VH comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008.

Also provided herein is an antigen binding polypeptide complex comprising a first polypeptide and a second polypeptide; wherein the first polypeptide has a structure represented by: VL1-L1-VL2-L2-VH2-L3-VH1-L4-Fc; VL1-L1-VH2-L2-VL2-L3-VH1-L4-Fc; VH1-L1-VH2-L2-VL2-L3-VL1-L4-Fc; or VH1-L1-VL2-L2-VH2-L3-VL1-L4-Fc; wherein the second polypeptide has a structure represented by: VL3-L5-VL4-L6-VH4-L7-VH3-L8-Fc; VL3-L5-VH4-L6-VL4-L7-VH3-L8-Fc; VH3-L5-VH4-L6-VL4-L7-VL3-L8-Fc; or VH3-L5-VL4-L6-VH4-L7-VL3-L8-Fc; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen, and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003; VL2 is a second immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003; VL3 is a third immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007; and VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007; Fc is a region comprising an immunoglobulin heavy chain constant region 2 (CH2), an immunoglobulin heavy chain constant region 3 (CH3), and optionally, an immunoglobulin hinge; and L1-L8 are optional amino acid linkers.

Also provided herein is an antigen binding polypeptide complex comprising a first polypeptide and a second polypeptide; wherein the first polypeptide has a structure represented by: VL1-L1-VL2-L2-VH2-L3-VH1-L4-Fc; VL1-L1-VH2-L2-VL2-L3-VH1-L4-Fc; VH1-L1-VH2-L2-VL2-37-VL1-L4-Fc; or VH1-L1-VL2-L2-VH2-L3-VL1-L4-Fc; wherein the second polypeptide has a structure represented by: VL3-L5-VL4-L6-VH4-L7-VH3-L8-Fc; VL3-L5-VH4-L6-VL4-L7-VH3-L8-Fc; VH3-L5-VH4-L6-VL4-L7-VL3-L8-Fc; or VH3-L5-VL4-L6-VH4-L7-VL3-L8-Fc; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL2 is a second immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL3 is a third immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; and VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; Fc is a region comprising an immunoglobulin heavy chain constant region 2 (CH2), an immunoglobulin heavy chain constant region 3 (CH3), and optionally, an immunoglobulin hinge; and L1-L8 are optional amino acid linkers.

In some aspects, the antigen binding polypeptide complex comprises a first polypeptide and a second polypeptide; wherein the first polypeptide has a structure represented by: VL1-L1-VL2-L2-VH2-L3-VH1-L4-Fc; or VH1-L1-VH2-L2-VL2-L3-VL1-L4-Fc; wherein the second polypeptide has a structure represented by: VL3-L5-VL4-L6-VH4-L7-VH3-L8-Fc; or VH3-L5-VH4-L6-VL4-L7-VL3-L8-Fc; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen, and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL1 is a first immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL2 is a second immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL2 is a second light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL3 is a third immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL3 is a third immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL4 is a fourth immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH3 is a third immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; and VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008. Fc is a region comprising an immunoglobulin heavy chain constant region 2 (CH2), an immunoglobulin heavy chain constant region 3 (CH3), and optionally, an immunoglobulin hinge; and L1-L8 are optional amino acid linkers.

Also provided herein is an antigen binding polypeptide complex comprising a first polypeptide and a second polypeptide; wherein the first polypeptide has a structure represented by: VL1-L1-VL2-L2-VH2-L3-VH1-L4-CH1-L5-CL-L6-Fc; VL1-L1-VH2-L2-VL2-L3-VH1-L4-CH1-L5-CL-L6-Fc; VH1-L1- VH2-L2-VL2-L3-VL1-L4-CH1-L5-CL-L6-Fc; VH1-L1-VL2-L2-VH2-L3-VL1-L4-CH1-L5-CL-L6-Fc; VL1-L1-VL2-L2-VH2-L3-VH1-L4-CL-L5-CH1-L6-Fc; VL1-L1-VH2-L2-VL2-L3-VH1-L4-CL-L5-CH1-L6-Fc; VH1-L1-VH2-L2-VL2-L3-VL1-L4-CL-L5-CH1-L6-Fc; or VH1-L1-VL2-L2-VH2-L3-VL1-L4- CL-L5-CH1-L6-Fc; wherein the second polypeptide has a structure represented by: VL3-L7-VL4-L8-VH4-L9-VH3-L10-CH1-L11-CL-L12-Fc; VL3-L7-VH4-L8-VL4-L9-VH3-L10-CH1-L11-CL-L12-Fc; VH3-L7-VH4-L8-VL4-L9-VL3-L10-CH1-L11-CL-L12-Fc; VH3-L7-VL4-L8-VH4-L9-VL3-L10-CH1-L11-CL- L12-Fc; VL3-L7-VL4-L8-VH4-L9-VH3-L10-CL-L11-CH1-L12-Fc; VL3-L7-VH4-L8-VL4-L9-VH3-L10-CL-L11-CH1-L12-Fc; VH3-L7-VH4-L8-VL4-L9-VL3-L10-CL-L11-CH1-L12-Fc; or VH3-L7-VL4-L8-VH4-L9-VL3-L10-CL-L11-CH1-L12-Fc; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen, and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL1 is a first immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL2 is a second immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL2 is a second light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL3 is a third immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL3 is a third immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL4 is a fourth immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH3 is a third immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; and VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L12 are optional amino acid linkers.

Also provided herein is an antigen binding polypeptide complex comprising two antigen binding polypeptides wherein at least one antigen binding polypeptide has a structure represented by: VL1-L1-VL2-L2-VH2-L3-VH1-L4-Fc-L5-Fc; VL1-L1-VH2-L2-VL2-L3-VH1-L4-Fc-L5-Fc; VH1-L1-VH2-L2- VL2-L3-VL1-L4-Fc-L5-Fc; or VH1-L1-VL2-L2-VH2-L3-VL1-L4-Fc-L5-Fc; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen, and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL1 is a first immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL2 is a second immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL2 is a second immunoglobulin light chain variable region that specifically binds (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; and VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; Fc is a region comprising an immunoglobulin heavy chain constant region 2 (CH2), an immunoglobulin heavy chain constant region 3 (CH3), and optionally, an immunoglobulin hinge; and L1-L5 are optional amino acid linkers.

In some aspects, the antigen binding polypeptide complex comprises a polypeptide having a structure represented by: VL1-L1-VL2-L2-VH2-L3-VH1-L4-Fc-L5-Fc; or VH1-L1-VH2-L2-VL2-L3-VL1-L4-Fc-L5-Fc; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen, and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL1 is a first immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL2 is a second immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001, and 1065; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL2 is a second immunoglobulin light chain variable region that specifically binds (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; and VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; Fc is a region comprising an immunoglobulin heavy chain constant region 2 (CH2), an immunoglobulin heavy chain constant region 3 (CH3), and optionally, an immunoglobulin hinge; and L1-L5 are optional amino acid linkers.

In some aspects, and in any one of the formulas shown herein, any one of the optional linkers each independently have a length of from 0 amino acids to about 50 amino acids.

In some aspects, and in any one of the formulas shown herein, any one of the optional linkers not having a length of 0 amino acids each independently comprise an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the Fc region comprises at least one knob-into-hole modification or Fc effector function knockout mutation. In some aspects, the antigen binding polypeptide complex disclosed herein is an IgG1 or IgG4 antibody or antigen binding fragment thereof and the knob-into-hole modification comprises: (i) knob substitutions of S354C and T366W; (ii) hole substitutions of Y349C, T366S, L368A and Y407V; (iii) a combination thereof; or equivalent thereof, based on the EU numbering scheme. In some aspects, the antigen binding polypeptide complex is an IgG1 or IgG4 antibody or antigen binding fragment thereof and the Fc effector function knockout mutation is L234A, L235A, P239A, or equivalent thereof, or a combination thereof, based on the EU numbering scheme.

In some aspects, the antigen binding polypeptide complex disclosed herein specifically binds to the (i) tumor-associated antigen (TAA), (ii) infectious disease antigen that is not a sarbecovirus antigen, or (iii) other-pathology antigen, with an equilibrium dissociation constant (K_{D})) of from about 10 μM to about 1 pM.

In some aspects, the antigen binding polypeptide complex disclosed herein is bispecific, trispecific or tetraspecific and has greater binding affinity to the (i) tumor-associated antigen (TAA), (ii) infectious disease antigen that is not a sarbecovirus antigen, or (iii) other-pathology antigen than a monospecific antigen binding polypeptide complex that specifically binds to one of the same antigens as the bispecific, trispecific or tetraspecific antigen binding polypeptide complex.

In some aspects, the antigen binding polypeptide complex disclosed herein is bispecific, trispecific or tetraspecific and has greater binding affinity to the (i) tumor-associated antigen (TAA), (ii) infectious disease antigen that is not a sarbecovirus antigen, or (iii) other-pathology antigen than a mixture of monospecific antigen binding polypeptide complexes that specifically bind to the same antigens as the bispecific, trispecific or tetraspecific antigen binding polypeptide complex.

Also provided herein is an antibody or an antigen binding fragment thereof comprising the antigen binding polypeptide or the antigen binding polypeptide complex disclosed herein, wherein the antibody or antigen binding fragment thereof exhibits improved prevention of immune escape compared to (i) a monovalent antibody or antigen binding fragment thereof that binds to one of the same antigens as the antibody or antigen binding fragment thereof, and/or (ii) a combination of monovalent antibodies or antigen binding fragments thereof that binds to the same antigens as the antibody or antigen binding fragment thereof.

Also provided herein is a pharmaceutical composition comprising the antigen binding polypeptide complex or the antibody or antigen binding fragment thereof disclosed herein and a pharmaceutically acceptable carrier.

Also provided herein is a kit comprising the antigen binding polypeptide complex, the antibody or antigen binding fragment thereof, or the pharmaceutical composition disclosed herein.

Also provided herein is a method of preventing or treating (i) a cancer or a tumor, (ii) an infectious disease that is not a sarbecovirus infectious disease, or (iii) a pathology other than a cancer or a tumor or an infectious disease that is not a sarbecovirus infectious disease in a subject in need thereof, comprising administering to the subject a therapeutically effective amount of the antigen binding polypeptide complex, the antibody or antigen binding fragment thereof, or the pharmaceutical composition disclosed herein.

Also provided herein is a method of diagnosing a subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptide complex or with the antibody or antigen binding fragment thereof disclosed herein; (b) detecting the presence or absence of a complex which contains the polypeptide complex or a fragment thereof, or the antibody or a fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is detected.

Also provided herein is a method of diagnosing a subject as not having or as not being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptide complex or with the antibody or antigen binding fragment thereof disclosed herein; (b) detecting the presence or absence of a complex which contains the polypeptide complex or a fragment thereof, or the antibody or a fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is not detected.

Also provided herein is an antibody or antigen binding fragment thereof selected from the group consisting of: (a) an antigen binding polypeptide having a structure represented by: VL1-L1-VH1 or VH1-L1-VL1; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; and L1 and L2 are optional amino acid linkers; or an antigen binding polypeptide having a structure represented by: VL1-L1-VL2-L2-VH2-L3-VH1; VL1-L1-VH2-L2-VL2-L3-VH1; VH1-L1-VH2-L2-VL2-L3-VL1; or VH1-L1-VL2-L2-VH2-L3-VL1; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen, and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL1 is a first immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL2 is a second immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL2 is a second immunoglobulin light chain variable region that specifically binds a (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VH1 is a first immunoglobulin heavy chain variable region that specifically binds to a (i) a tumor-associated antigen (TAA), (ii) an infectious disease antigen that is not a sarbecovirus antigen, or (iii) an other-pathology antigen, and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; and VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; L1-L3 are optional amino acid linkers; and wherein (a) is selected from any one of the more specific embodiments provided herein for an antigen binding polypeptide in an antigen binding polypeptide complex having no more than three polypeptide chains; (b) an antigen binding polypeptide having a structure represented by: VL1-L1-VH1 or VH1-L1-VL1; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen, and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL1 is a first immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; and L1 and L2 are optional amino acid linkers; or an antigen binding polypeptide having a structure represented by: VL1-L1-VL2-L2-VH2-L3-VH1; VL1-L1-VH2-L2-VL2-L3-VH1; VH1-L1-VH2-L2-VL2-L3-VL1; or VH1-L1-VL2-L2-VH2-L3-VL1; wherein: VL1 is a first immunoglobulin light chain variable region that specifically binds to (i) a tumor-associated antigen (TAA), (ii) an infectious disease antigen that is not a sarbecovirus antigen, or (iii) an other-pathology antigen, and which comprises a light chain complementary determining region (LCDR) 1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL1 is a first immunoglobulin light chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VL2 is a second immunoglobulin light chain variable region that specifically binds to (i) a tumor-associated antigen (TAA), (ii) an infectious disease antigen that is not a sarbecovirus antigen, or (iii) an other-pathology antigen, and which comprises a LCDR1 comprising an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001; a LCDR2 comprising an amino acid sequence at least 65%, at least 70%, at least 75%, at least 80%, at least 85%, at least 90%, at least 95%, at least 99%, or 100% identical to any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS, or at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, 1777, 1978, 1986, 1994, and 2002; and a LCDR3 comprising an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003, or VL2 is a second immunoglobulin light chain variable region that specifically binds (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, and which comprises a heavy chain complementary determining region (HCDR) 1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH1 is a first immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; and VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, and which comprises a HCDR1 comprising an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005; a HCDR2 comprising an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006; and a HCDR3 comprising an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007, or VH2 is a second immunoglobulin heavy chain variable region that specifically binds to (i) a tumor-associated antigen (TAA), (ii) an infectious disease antigen that is not a sarbecovirus antigen, or (iii) an other-pathology antigen, comprising an amino acid sequence at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008; and L1-L3 are optional amino acid linkers; wherein (b) is selected from any one of the more specific embodiments provided herein for an antigen binding polypeptide in an antigen binding polypeptide complex having no more than three polypeptide chains; and wherein the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide, antigen binding polypeptide complex, antibody or antigen binding fragment thereof, polynucleotide, vector, host cell, mRNA, LNP, CAR, immune cell, pharmaceutical composition, kit, or method disclosed herein comprises or encodes:
a VL at least 65%, at least 70%, at least 75%, at least 80%, at least 85%, at least 90%, at least 95%, at least 99%, or 100% identical to SEQ ID NO: 1098 and a VH at least 65%, at least 70%, at least 75%, at least 80%, at least 85%, at least 90%, at least 95%, at least 99%, or 100% identical to SEQ ID NO: 1102;
a VL at least 65%, at least 70%, at least 75%, at least 80%, at least 85%, at least 90%, at least 95%, at least 99%, or 100% identical to SEQ ID NO: 1105 and a VH at least 65%, at least 70%, at least 75%, at least 80%, at least 85%, at least 90%, at least 95%, at least 99%, or 100% identical to SEQ ID NO: 1109;
a VL at least 65%, at least 70%, at least 75%, at least 80%, at least 85%, at least 90%, at least 95%, at least 99%, or 100% identical to SEQ ID NO: 1112 and a VH at least 65%, at least 70%, at least 75%, at least 80%, at least 85%, at least 90%, at least 95%, at least 99%, or 100% identical to SEQ ID NO: 1116;
a VL at least 65%, at least 70%, at least 75%, at least 80%, at least 85%, at least 90%, at least 95%, at least 99%, or 100% identical to SEQ ID NO: 1105 and a VH at least 65%, at least 70%, at least 75%, at least 80%, at least 85%, at least 90%, at least 95%, at least 99%, or 100% identical to SEQ ID NO: 1119;
a VL at least 65%, at least 70%, at least 75%, at least 80%, at least 85%, at least 90%, at least 95%, at least 99%, or 100% identical to SEQ ID NO: 1122 and a VH at least 65%, at least 70%, at least 75%, at least 80%, at least 85%, at least 90%, at least 95%, at least 99%, or 100% identical to SEQ ID NO: 1126;
a VL at least 65%, at least 70%, at least 75%, at least 80%, at least 85%, at least 90%, at least 95%, at least 99%, or 100% identical to SEQ ID NO: 1129 and a VH at least 65%, at least 70%, at least 75%, at least 80%, at least 85%, at least 90%, at least 95%, at least 99%, or 100% identical to SEQ ID NO: 1133;
a VL at least 65%, at least 70%, at least 75%, at least 80%, at least 85%, at least 90%, at least 95%, at least 99%, or 100% identical to SEQ ID NO: 1136 and a VH at least 65%, at least 70%, at least 75%, at least 80%, at least 85%, at least 90%, at least 95%, at least 99%, or 100% identical to SEQ ID NO: 1140;
a VL at least 65%, at least 70%, at least 75%, at least 80%, at least 85%, at least 90%, at least 95%, at least 99%, or 100% identical to SEQ ID NO: 1143 and a VH at least 65%, at least 70%, at least 75%, at least 80%, at least 85%, at least 90%, at least 95%, at least 99%, or 100% identical to SEQ ID NO: 1147;
a VL at least 65%, at least 70%, at least 75%, at least 80%, at least 85%, at least 90%, at least 95%, at least 99%, or 100% identical to SEQ ID NO: 1150 and a VH at least 65%, at least 70%, at least 75%, at least 80%, at least 85%, at least 90%, at least 95%, at least 99%, or 100% identical to SEQ ID NO: 1154;
a VL at least 65%, at least 70%, at least 75%, at least 80%, at least 85%, at least 90%, at least 95%, at least 99%, or 100% identical to SEQ ID NO: 1157 and a VH at least 65%, at least 70%, at least 75%, at least 80%, at least 85%, at least 90%, at least 95%, at least 99%, or 100% identical to SEQ ID NO: 1161;
a VL at least 65%, at least 70%, at least 75%, at least 80%, at least 85%, at least 90%, at least 95%, at least 99%, or 100% identical to SEQ ID NO: 1164 and a VH at least 65%, at least 70%, at least 75%, at least 80%, at least 85%, at least 90%, at least 95%, at least 99%, or 100% identical to SEQ ID NO: 1168;
a VL at least 65%, at least 70%, at least 75%, at least 80%, at least 85%, at least 90%, at least 95%, at least 99%, or 100% identical to SEQ ID NO: 1171 and a VH at least 65%, at least 70%, at least 75%, at least 80%, at least 85%, at least 90%, at least 95%, at least 99%, or 100% identical to SEQ ID NO: 1173;
a VL at least 65%, at least 70%, at least 75%, at least 80%, at least 85%, at least 90%, at least 95%, at least 99%, or 100% identical to SEQ ID NO: 1176 and a VH at least 65%, at least 70%, at least 75%, at least 80%, at least 85%, at least 90%, at least 95%, at least 99%, or 100% identical to SEQ ID NO: 1180;
a VL at least 65%, at least 70%, at least 75%, at least 80%, at least 85%, at least 90%, at least 95%, at least 99%, or 100% identical to SEQ ID NO: 1183 and a VH at least 65%, at least 70%, at least 75%, at least 80%, at least 85%, at least 90%, at least 95%, at least 99%, or 100% identical to SEQ ID NO: 1187;
a VL at least 65%, at least 70%, at least 75%, at least 80%, at least 85%, at least 90%, at least 95%, at least 99%, or 100% identical to SEQ ID NO: 1190 and a VH at least 65%, at least 70%, at least 75%, at least 80%, at least 85%, at least 90%, at least 95%, at least 99%, or 100% identical to SEQ ID NO: 1194;
a VL at least 65%, at least 70%, at least 75%, at least 80%, at least 85%, at least 90%, at least 95%, at least 99%, or 100% identical to SEQ ID NO: 1196 and a VH at least 65%, at least 70%, at least 75%, at least 80%, at least 85%, at least 90%, at least 95%, at least 99%, or 100% identical to SEQ ID NO: 1198;
a VL at least 65%, at least 70%, at least 75%, at least 80%, at least 85%, at least 90%, at least 95%, at least 99%, or 100% identical to SEQ ID NO: 1202 and a VH at least 65%, at least 70%, at least 75%, at least 80%, at least 85%, at least 90%, at least 95%, at least 99%, or 100% identical to SEQ ID NO: 1206;
a VL at least 65%, at least 70%, at least 75%, at least 80%, at least 85%, at least 90%, at least 95%, at least 99%, or 100% identical to SEQ ID NO: 1208 and a VH at least 65%, at least 70%, at least 75%, at least 80%, at least 85%, at least 90%, at least 95%, at least 99%, or 100% identical to SEQ ID NO: 1210;
a VL at least 65%, at least 70%, at least 75%, at least 80%, at least 85%, at least 90%, at least 95%, at least 99%, or 100% identical to SEQ ID NO: 1214 and a VH at least 65%, at least 70%, at least 75%, at least 80%, at least 85%, at least 90%, at least 95%, at least 99%, or 100% identical to SEQ ID NO: 1218;
a VL at least 65%, at least 70%, at least 75%, at least 80%, at least 85%, at least 90%, at least 95%, at least 99%, or 100% identical to SEQ ID NO: 1219 and a VH at least 65%, at least 70%, at least 75%, at least 80%, at least 85%, at least 90%, at least 95%, at least 99%, or 100% identical to SEQ ID NO: 1220;
a VL at least 65%, at least 70%, at least 75%, at least 80%, at least 85%, at least 90%, at least 95%, at least 99%, or 100% identical to SEQ ID NO: 1223 and a VH at least 65%, at least 70%, at least 75%, at least 80%, at least 85%, at least 90%, at least 95%, at least 99%, or 100% identical to SEQ ID NO: 1227;
a VL at least 65%, at least 70%, at least 75%, at least 80%, at least 85%, at least 90%, at least 95%, at least 99%, or 100% identical to SEQ ID NO: 1230 and a VH at least 65%, at least 70%, at least 75%, at least 80%, at least 85%, at least 90%, at least 95%, at least 99%, or 100% identical to SEQ ID NO: 1234;
a VL at least 65%, at least 70%, at least 75%, at least 80%, at least 85%, at least 90%, at least 95%, at least 99%, or 100% identical to SEQ ID NO: 1237 and a VH at least 65%, at least 70%, at least 75%, at least 80%, at least 85%, at least 90%, at least 95%, at least 99%, or 100% identical to SEQ ID NO: 1241;
a VL at least 65%, at least 70%, at least 75%, at least 80%, at least 85%, at least 90%, at least 95%, at least 99%, or 100% identical to SEQ ID NO: 1242 and a VH at least 65%, at least 70%, at least 75%, at least 80%, at least 85%, at least 90%, at least 95%, at least 99%, or 100% identical to SEQ ID NO: 1246;
a VL at least 65%, at least 70%, at least 75%, at least 80%, at least 85%, at least 90%, at least 95%, at least 99%, or 100% identical to SEQ ID NO: 1249 and a VH at least 65%, at least 70%, at least 75%, at least 80%, at least 85%, at least 90%, at least 95%, at least 99%, or 100% identical to SEQ ID NO: 1253;
a VL at least 65%, at least 70%, at least 75%, at least 80%, at least 85%, at least 90%, at least 95%, at least 99%, or 100% identical to SEQ ID NO: 1256 and a VH at least 65%, at least 70%, at least 75%, at least 80%, at least 85%, at least 90%, at least 95%, at least 99%, or 100% identical to SEQ ID NO: 1260;
a VL at least 65%, at least 70%, at least 75%, at least 80%, at least 85%, at least 90%, at least 95%, at least 99%, or 100% identical to SEQ ID NO: 1263 and a VH at least 65%, at least 70%, at least 75%, at least 80%, at least 85%, at least 90%, at least 95%, at least 99%, or 100% identical to SEQ ID NO: 1265;
a VL at least 65%, at least 70%, at least 75%, at least 80%, at least 85%, at least 90%, at least 95%, at least 99%, or 100% identical to SEQ ID NO: 1268 and a VH at least 65%, at least 70%, at least 75%, at least 80%, at least 85%, at least 90%, at least 95%, at least 99%, or 100% identical to SEQ ID NO: 1270;
a VL at least 65%, at least 70%, at least 75%, at least 80%, at least 85%, at least 90%, at least 95%, at least 99%, or 100% identical to SEQ ID NO: 1273 and a VH at least 65%, at least 70%, at least 75%, at least 80%, at least 85%, at least 90%, at least 95%, at least 99%, or 100% identical to SEQ ID NO: 1277;
a VL at least 65%, at least 70%, at least 75%, at least 80%, at least 85%, at least 90%, at least 95%, at least 99%, or 100% identical to SEQ ID NO: 1280 and a VH at least 65%, at least 70%, at least 75%, at least 80%, at least 85%, at least 90%, at least 95%, at least 99%, or 100% identical to SEQ ID NO: 1284;
a VL at least 65%, at least 70%, at least 75%, at least 80%, at least 85%, at least 90%, at least 95%, at least 99%, or 100% identical to SEQ ID NO: 1287 and a VH at least 65%, at least 70%, at least 75%, at least 80%, at least 85%, at least 90%, at least 95%, at least 99%, or 100% identical to SEQ ID NO: 1291;
a VL at least 65%, at least 70%, at least 75%, at least 80%, at least 85%, at least 90%, at least 95%, at least 99%, or 100% identical to SEQ ID NO: 1294 and a VH at least 65%, at least 70%, at least 75%, at least 80%, at least 85%, at least 90%, at least 95%, at least 99%, or 100% identical to SEQ ID NO: 1298;
a VL at least 65%, at least 70%, at least 75%, at least 80%, at least 85%, at least 90%, at least 95%, at least 99%, or 100% identical to SEQ ID NO: 1301 and a VH at least 65%, at least 70%, at least 75%, at least 80%, at least 85%, at least 90%, at least 95%, at least 99%, or 100% identical to SEQ ID NO: 1305;
a VL at least 65%, at least 70%, at least 75%, at least 80%, at least 85%, at least 90%, at least 95%, at least 99%, or 100% identical to SEQ ID NO: 1308 and a VH at least 65%, at least 70%, at least 75%, at least 80%, at least 85%, at least 90%, at least 95%, at least 99%, or 100% identical to SEQ ID NO: 1312;
a VL at least 65%, at least 70%, at least 75%, at least 80%, at least 85%, at least 90%, at least 95%, at least 99%, or 100% identical to SEQ ID NO: 1314 and a VH at least 65%, at least 70%, at least 75%, at least 80%, at least 85%, at least 90%, at least 95%, at least 99%, or 100% identical to SEQ ID NO: 1318;
a VL at least 65%, at least 70%, at least 75%, at least 80%, at least 85%, at least 90%, at least 95%, at least 99%, or 100% identical to SEQ ID NO: 1321 and a VH at least 65%, at least 70%, at least 75%, at least 80%, at least 85%, at least 90%, at least 95%, at least 99%, or 100% identical to SEQ ID NO: 1325;
a VL at least 65%, at least 70%, at least 75%, at least 80%, at least 85%, at least 90%, at least 95%, at least 99%, or 100% identical to SEQ ID NO: 1328 and a VH at least 65%, at least 70%, at least 75%, at least 80%, at least 85%, at least 90%, at least 95%, at least 99%, or 100% identical to SEQ ID NO: 1332;
a VL at least 65%, at least 70%, at least 75%, at least 80%, at least 85%, at least 90%, at least 95%, at least 99%, or 100% identical to SEQ ID NO: 1334 and a VH at least 65%, at least 70%, at least 75%, at least 80%, at least 85%, at least 90%, at least 95%, at least 99%, or 100% identical to SEQ ID NO: 1338;
a VL at least 65%, at least 70%, at least 75%, at least 80%, at least 85%, at least 90%, at least 95%, at least 99%, or 100% identical to SEQ ID NO: 1341 and a VH at least 65%, at least 70%, at least 75%, at least 80%, at least 85%, at least 90%, at least 95%, at least 99%, or 100% identical to SEQ ID NO: 1345;
a VL at least 65%, at least 70%, at least 75%, at least 80%, at least 85%, at least 90%, at least 95%, at least 99%, or 100% identical to SEQ ID NO: 1347 and a VH at least 65%, at least 70%, at least 75%, at least 80%, at least 85%, at least 90%, at least 95%, at least 99%, or 100% identical to SEQ ID NO: 1351;
a VL at least 65%, at least 70%, at least 75%, at least 80%, at least 85%, at least 90%, at least 95%, at least 99%, or 100% identical to SEQ ID NO: 1354 and a VH at least 65%, at least 70%, at least 75%, at least 80%, at least 85%, at least 90%, at least 95%, at least 99%, or 100% identical to SEQ ID NO: 1358;
a VL at least 65%, at least 70%, at least 75%, at least 80%, at least 85%, at least 90%, at least 95%, at least 99%, or 100% identical to SEQ ID NO: 1361 and a VH at least 65%, at least 70%, at least 75%, at least 80%, at least 85%, at least 90%, at least 95%, at least 99%, or 100% identical to SEQ ID NO: 1365;
a VL at least 65%, at least 70%, at least 75%, at least 80%, at least 85%, at least 90%, at least 95%, at least 99%, or 100% identical to SEQ ID NO: 1368 and a VH at least 65%, at least 70%, at least 75%, at least 80%, at least 85%, at least 90%, at least 95%, at least 99%, or 100% identical to SEQ ID NO: 1372;
a VL at least 65%, at least 70%, at least 75%, at least 80%, at least 85%, at least 90%, at least 95%, at least 99%, or 100% identical to SEQ ID NO: 1375 and a VH at least 65%, at least 70%, at least 75%, at least 80%, at least 85%, at least 90%, at least 95%, at least 99%, or 100% identical to SEQ ID NO: 1379;
a VL at least 65%, at least 70%, at least 75%, at least 80%, at least 85%, at least 90%, at least 95%, at least 99%, or 100% identical to SEQ ID NO: 1380 and a VH at least 65%, at least 70%, at least 75%, at least 80%, at least 85%, at least 90%, at least 95%, at least 99%, or 100% identical to SEQ ID NO: 1381;
a VL at least 65%, at least 70%, at least 75%, at least 80%, at least 85%, at least 90%, at least 95%, at least 99%, or 100% identical to SEQ ID NO: 1384 and a VH at least 65%, at least 70%, at least 75%, at least 80%, at least 85%, at least 90%, at least 95%, at least 99%, or 100% identical to SEQ ID NO: 1388;
a VL at least 65%, at least 70%, at least 75%, at least 80%, at least 85%, at least 90%, at least 95%, at least 99%, or 100% identical to SEQ ID NO: 1390 and a VH at least 65%, at least 70%, at least 75%, at least 80%, at least 85%, at least 90%, at least 95%, at least 99%, or 100% identical to SEQ ID NO: 1393;
a VL at least 65%, at least 70%, at least 75%, at least 80%, at least 85%, at least 90%, at least 95%, at least 99%, or 100% identical to SEQ ID NO: 1396 and a VH at least 65%, at least 70%, at least 75%, at least 80%, at least 85%, at least 90%, at least 95%, at least 99%, or 100% identical to SEQ ID NO: 1400;
a VL at least 65%, at least 70%, at least 75%, at least 80%, at least 85%, at least 90%, at least 95%, at least 99%, or 100% identical to SEQ ID NO: 1403 and a VH at least 65%, at least 70%, at least 75%, at least 80%, at least 85%, at least 90%, at least 95%, at least 99%, or 100% identical to SEQ ID NO: 1407;
a VL at least 65%, at least 70%, at least 75%, at least 80%, at least 85%, at least 90%, at least 95%, at least 99%, or 100% identical to SEQ ID NO: 1410 and a VH at least 65%, at least 70%, at least 75%, at least 80%, at least 85%, at least 90%, at least 95%, at least 99%, or 100% identical to SEQ ID NO: 1413;
a VL at least 65%, at least 70%, at least 75%, at least 80%, at least 85%, at least 90%, at least 95%, at least 99%, or 100% identical to SEQ ID NO: 1416 and a VH at least 65%, at least 70%, at least 75%, at least 80%, at least 85%, at least 90%, at least 95%, at least 99%, or 100% identical to SEQ ID NO: 1420;
a VL at least 65%, at least 70%, at least 75%, at least 80%, at least 85%, at least 90%, at least 95%, at least 99%, or 100% identical to SEQ ID NO: 1423 and a VH at least 65%, at least 70%, at least 75%, at least 80%, at least 85%, at least 90%, at least 95%, at least 99%, or 100% identical to SEQ ID NO: 1427;
a VL at least 65%, at least 70%, at least 75%, at least 80%, at least 85%, at least 90%, at least 95%, at least 99%, or 100% identical to SEQ ID NO: 1430 and a VH at least 65%, at least 70%, at least 75%, at least 80%, at least 85%, at least 90%, at least 95%, at least 99%, or 100% identical to SEQ ID NO: 1434;
a VL at least 65%, at least 70%, at least 75%, at least 80%, at least 85%, at least 90%, at least 95%, at least 99%, or 100% identical to SEQ ID NO: 1436 and a VH at least 65%, at least 70%, at least 75%, at least 80%, at least 85%, at least 90%, at least 95%, at least 99%, or 100% identical to SEQ ID NO: 1440;
a VL at least 65%, at least 70%, at least 75%, at least 80%, at least 85%, at least 90%, at least 95%, at least 99%, or 100% identical to SEQ ID NO: 1443 and a VH at least 65%, at least 70%, at least 75%, at least 80%, at least 85%, at least 90%, at least 95%, at least 99%, or 100% identical to SEQ ID NO: 1447;
a VL at least 65%, at least 70%, at least 75%, at least 80%, at least 85%, at least 90%, at least 95%, at least 99%, or 100% identical to SEQ ID NO: 1450 and a VH at least 65%, at least 70%, at least 75%, at least 80%, at least 85%, at least 90%, at least 95%, at least 99%, or 100% identical to SEQ ID NO: 1454;
a VL at least 65%, at least 70%, at least 75%, at least 80%, at least 85%, at least 90%, at least 95%, at least 99%, or 100% identical to SEQ ID NO: 1457 and a VH at least 65%, at least 70%, at least 75%, at least 80%, at least 85%, at least 90%, at least 95%, at least 99%, or 100% identical to SEQ ID NO: 1460;
a VL at least 65%, at least 70%, at least 75%, at least 80%, at least 85%, at least 90%, at least 95%, at least 99%, or 100% identical to SEQ ID NO: 1463 and a VH at least 65%, at least 70%, at least 75%, at least 80%, at least 85%, at least 90%, at least 95%, at least 99%, or 100% identical to SEQ ID NO: 1467;
a VL at least 65%, at least 70%, at least 75%, at least 80%, at least 85%, at least 90%, at least 95%, at least 99%, or 100% identical to SEQ ID NO: 1470 and a VH at least 65%, at least 70%, at least 75%, at least 80%, at least 85%, at least 90%, at least 95%, at least 99%, or 100% identical to SEQ ID NO: 1474;
a VL at least 65%, at least 70%, at least 75%, at least 80%, at least 85%, at least 90%, at least 95%, at least 99%, or 100% identical to SEQ ID NO: 1477 and a VH at least 65%, at least 70%, at least 75%, at least 80%, at least 85%, at least 90%, at least 95%, at least 99%, or 100% identical to SEQ ID NO: 1481;
a VL at least 65%, at least 70%, at least 75%, at least 80%, at least 85%, at least 90%, at least 95%, at least 99%, or 100% identical to SEQ ID NO: 1483 and a VH at least 65%, at least 70%, at least 75%, at least 80%, at least 85%, at least 90%, at least 95%, at least 99%, or 100% identical to SEQ ID NO: 1487;
a VL at least 65%, at least 70%, at least 75%, at least 80%, at least 85%, at least 90%, at least 95%, at least 99%, or 100% identical to SEQ ID NO: 1490 and a VH at least 65%, at least 70%, at least 75%, at least 80%, at least 85%, at least 90%, at least 95%, at least 99%, or 100% identical to SEQ ID NO: 1494;
a VL at least 65%, at least 70%, at least 75%, at least 80%, at least 85%, at least 90%, at least 95%, at least 99%, or 100% identical to SEQ ID NO: 1497 and a VH at least 65%, at least 70%, at least 75%, at least 80%, at least 85%, at least 90%, at least 95%, at least 99%, or 100% identical to SEQ ID NO: 1501;
a VL at least 65%, at least 70%, at least 75%, at least 80%, at least 85%, at least 90%, at least 95%, at least 99%, or 100% identical to SEQ ID NO: 1503 and a VH at least 65%, at least 70%, at least 75%, at least 80%, at least 85%, at least 90%, at least 95%, at least 99%, or 100% identical to SEQ ID NO: 1507;
a VL at least 65%, at least 70%, at least 75%, at least 80%, at least 85%, at least 90%, at least 95%, at least 99%, or 100% identical to SEQ ID NO: 1510 and a VH at least 65%, at least 70%, at least 75%, at least 80%, at least 85%, at least 90%, at least 95%, at least 99%, or 100% identical to SEQ ID NO: 1512;
a VL at least 65%, at least 70%, at least 75%, at least 80%, at least 85%, at least 90%, at least 95%, at least 99%, or 100% identical to SEQ ID NO: 1514 and a VH at least 65%, at least 70%, at least 75%, at least 80%, at least 85%, at least 90%, at least 95%, at least 99%, or 100% identical to SEQ ID NO: 1518;
a VL at least 65%, at least 70%, at least 75%, at least 80%, at least 85%, at least 90%, at least 95%, at least 99%, or 100% identical to SEQ ID NO: 1521 and a VH at least 65%, at least 70%, at least 75%, at least 80%, at least 85%, at least 90%, at least 95%, at least 99%, or 100% identical to SEQ ID NO: 1525;
a VL at least 65%, at least 70%, at least 75%, at least 80%, at least 85%, at least 90%, at least 95%, at least 99%, or 100% identical to SEQ ID NO: 1527 and a VH at least 65%, at least 70%, at least 75%, at least 80%, at least 85%, at least 90%, at least 95%, at least 99%, or 100% identical to SEQ ID NO: 1530;
a VL at least 65%, at least 70%, at least 75%, at least 80%, at least 85%, at least 90%, at least 95%, at least 99%, or 100% identical to SEQ ID NO: 1533 and a VH at least 65%, at least 70%, at least 75%, at least 80%, at least 85%, at least 90%, at least 95%, at least 99%, or 100% identical to SEQ ID NO: 1537;
a VL at least 65%, at least 70%, at least 75%, at least 80%, at least 85%, at least 90%, at least 95%, at least 99%, or 100% identical to SEQ ID NO: 1540 and a VH at least 65%, at least 70%, at least 75%, at least 80%, at least 85%, at least 90%, at least 95%, at least 99%, or 100% identical to SEQ ID NO: 1543;
a VL at least 65%, at least 70%, at least 75%, at least 80%, at least 85%, at least 90%, at least 95%, at least 99%, or 100% identical to SEQ ID NO: 1546 and a VH at least 65%, at least 70%, at least 75%, at least 80%, at least 85%, at least 90%, at least 95%, at least 99%, or 100% identical to SEQ ID NO: 1550;
a VL at least 65%, at least 70%, at least 75%, at least 80%, at least 85%, at least 90%, at least 95%, at least 99%, or 100% identical to SEQ ID NO: 1551 and a VH at least 65%, at least 70%, at least 75%, at least 80%, at least 85%, at least 90%, at least 95%, at least 99%, or 100% identical to SEQ ID NO: 1555;
a VL at least 65%, at least 70%, at least 75%, at least 80%, at least 85%, at least 90%, at least 95%, at least 99%, or 100% identical to SEQ ID NO: 1558 and a VH at least 65%, at least 70%, at least 75%, at least 80%, at least 85%, at least 90%, at least 95%, at least 99%, or 100% identical to SEQ ID NO: 1561;
a VL at least 65%, at least 70%, at least 75%, at least 80%, at least 85%, at least 90%, at least 95%, at least 99%, or 100% identical to SEQ ID NO: 1563 and a VH at least 65%, at least 70%, at least 75%, at least 80%, at least 85%, at least 90%, at least 95%, at least 99%, or 100% identical to SEQ ID NO: 1567;
a VL at least 65%, at least 70%, at least 75%, at least 80%, at least 85%, at least 90%, at least 95%, at least 99%, or 100% identical to SEQ ID NO: 1570 and a VH at least 65%, at least 70%, at least 75%, at least 80%, at least 85%, at least 90%, at least 95%, at least 99%, or 100% identical to SEQ ID NO: 1573;
a VL at least 65%, at least 70%, at least 75%, at least 80%, at least 85%, at least 90%, at least 95%, at least 99%, or 100% identical to SEQ ID NO: 1576 and a VH at least 65%, at least 70%, at least 75%, at least 80%, at least 85%, at least 90%, at least 95%, at least 99%, or 100% identical to SEQ ID NO: 1580;
a VL at least 65%, at least 70%, at least 75%, at least 80%, at least 85%, at least 90%, at least 95%, at least 99%, or 100% identical to SEQ ID NO: 1583 and a VH at least 65%, at least 70%, at least 75%, at least 80%, at least 85%, at least 90%, at least 95%, at least 99%, or 100% identical to SEQ ID NO: 1587;
a VL at least 65%, at least 70%, at least 75%, at least 80%, at least 85%, at least 90%, at least 95%, at least 99%, or 100% identical to SEQ ID NO: 1589 and a VH at least 65%, at least 70%, at least 75%, at least 80%, at least 85%, at least 90%, at least 95%, at least 99%, or 100% identical to SEQ ID NO: 1593;
a VL at least 65%, at least 70%, at least 75%, at least 80%, at least 85%, at least 90%, at least 95%, at least 99%, or 100% identical to SEQ ID NO: 1596 and a VH at least 65%, at least 70%, at least 75%, at least 80%, at least 85%, at least 90%, at least 95%, at least 99%, or 100% identical to SEQ ID NO: 1600;
a VL at least 65%, at least 70%, at least 75%, at least 80%, at least 85%, at least 90%, at least 95%, at least 99%, or 100% identical to SEQ ID NO: 1603 and a VH at least 65%, at least 70%, at least 75%, at least 80%, at least 85%, at least 90%, at least 95%, at least 99%, or 100% identical to SEQ ID NO: 1607;
a VL at least 65%, at least 70%, at least 75%, at least 80%, at least 85%, at least 90%, at least 95%, at least 99%, or 100% identical to SEQ ID NO: 1610 and a VH at least 65%, at least 70%, at least 75%, at least 80%, at least 85%, at least 90%, at least 95%, at least 99%, or 100% identical to SEQ ID NO: 1614;
a VL at least 65%, at least 70%, at least 75%, at least 80%, at least 85%, at least 90%, at least 95%, at least 99%, or 100% identical to SEQ ID NO: 1617 and a VH at least 65%, at least 70%, at least 75%, at least 80%, at least 85%, at least 90%, at least 95%, at least 99%, or 100% identical to SEQ ID NO: 1621;
a VL at least 65%, at least 70%, at least 75%, at least 80%, at least 85%, at least 90%, at least 95%, at least 99%, or 100% identical to SEQ ID NO: 1624 and a VH at least 65%, at least 70%, at least 75%, at least 80%, at least 85%, at least 90%, at least 95%, at least 99%, or 100% identical to SEQ ID NO: 1628;
a VL at least 65%, at least 70%, at least 75%, at least 80%, at least 85%, at least 90%, at least 95%, at least 99%, or 100% identical to SEQ ID NO: 1631 and a VH at least 65%, at least 70%, at least 75%, at least 80%, at least 85%, at least 90%, at least 95%, at least 99%, or 100% identical to SEQ ID NO: 1635;
a VL at least 65%, at least 70%, at least 75%, at least 80%, at least 85%, at least 90%, at least 95%, at least 99%, or 100% identical to SEQ ID NO: 1638 and a VH at least 65%, at least 70%, at least 75%, at least 80%, at least 85%, at least 90%, at least 95%, at least 99%, or 100% identical to SEQ ID NO: 1642;
a VL at least 65%, at least 70%, at least 75%, at least 80%, at least 85%, at least 90%, at least 95%, at least 99%, or 100% identical to SEQ ID NO: 1645 and a VH at least 65%, at least 70%, at least 75%, at least 80%, at least 85%, at least 90%, at least 95%, at least 99%, or 100% identical to SEQ ID NO: 1649;
a VL at least 65%, at least 70%, at least 75%, at least 80%, at least 85%, at least 90%, at least 95%, at least 99%, or 100% identical to SEQ ID NO: 1652 and a VH at least 65%, at least 70%, at least 75%, at least 80%, at least 85%, at least 90%, at least 95%, at least 99%, or 100% identical to SEQ ID NO: 1656;
a VL at least 65%, at least 70%, at least 75%, at least 80%, at least 85%, at least 90%, at least 95%, at least 99%, or 100% identical to SEQ ID NO: 1659 and a VH at least 65%, at least 70%, at least 75%, at least 80%, at least 85%, at least 90%, at least 95%, at least 99%, or 100% identical to SEQ ID NO: 1662;
a VL at least 65%, at least 70%, at least 75%, at least 80%, at least 85%, at least 90%, at least 95%, at least 99%, or 100% identical to SEQ ID NO: 1664 and a VH at least 65%, at least 70%, at least 75%, at least 80%, at least 85%, at least 90%, at least 95%, at least 99%, or 100% identical to SEQ ID NO: 1668;
a VL at least 65%, at least 70%, at least 75%, at least 80%, at least 85%, at least 90%, at least 95%, at least 99%, or 100% identical to SEQ ID NO: 1669 and a VH at least 65%, at least 70%, at least 75%, at least 80%, at least 85%, at least 90%, at least 95%, at least 99%, or 100% identical to SEQ ID NO: 1671;
a VL at least 65%, at least 70%, at least 75%, at least 80%, at least 85%, at least 90%, at least 95%, at least 99%, or 100% identical to SEQ ID NO: 1674 and a VH at least 65%, at least 70%, at least 75%, at least 80%, at least 85%, at least 90%, at least 95%, at least 99%, or 100% identical to SEQ ID NO: 1678;
a VL at least 65%, at least 70%, at least 75%, at least 80%, at least 85%, at least 90%, at least 95%, at least 99%, or 100% identical to SEQ ID NO: 1682 and a VH at least 65%, at least 70%, at least 75%, at least 80%, at least 85%, at least 90%, at least 95%, at least 99%, or 100% identical to SEQ ID NO: 1686;
a VL at least 65%, at least 70%, at least 75%, at least 80%, at least 85%, at least 90%, at least 95%, at least 99%, or 100% identical to SEQ ID NO: 1692 and a VH at least 65%, at least 70%, at least 75%, at least 80%, at least 85%, at least 90%, at least 95%, at least 99%, or 100% identical to SEQ ID NO: 1696;
a VL at least 65%, at least 70%, at least 75%, at least 80%, at least 85%, at least 90%, at least 95%, at least 99%, or 100% identical to SEQ ID NO: 1700 and a VH at least 65%, at least 70%, at least 75%, at least 80%, at least 85%, at least 90%, at least 95%, at least 99%, or 100% identical to SEQ ID NO: 1704;
a VL at least 65%, at least 70%, at least 75%, at least 80%, at least 85%, at least 90%, at least 95%, at least 99%, or 100% identical to SEQ ID NO: 1711 and a VH at least 65%, at least 70%, at least 75%, at least 80%, at least 85%, at least 90%, at least 95%, at least 99%, or 100% identical to SEQ ID NO: 1714;
a VL at least 65%, at least 70%, at least 75%, at least 80%, at least 85%, at least 90%, at least 95%, at least 99%, or 100% identical to SEQ ID NO: 1721 and a VH at least 65%, at least 70%, at least 75%, at least 80%, at least 85%, at least 90%, at least 95%, at least 99%, or 100% identical to SEQ ID NO: 1722;
a VL at least 65%, at least 70%, at least 75%, at least 80%, at least 85%, at least 90%, at least 95%, at least 99%, or 100% identical to SEQ ID NO: 1724 and a VH at least 65%, at least 70%, at least 75%, at least 80%, at least 85%, at least 90%, at least 95%, at least 99%, or 100% identical to SEQ ID NO: 1726;
a VL at least 65%, at least 70%, at least 75%, at least 80%, at least 85%, at least 90%, at least 95%, at least 99%, or 100% identical to SEQ ID NO: 1730 and a VH at least 65%, at least 70%, at least 75%, at least 80%, at least 85%, at least 90%, at least 95%, at least 99%, or 100% identical to SEQ ID NO: 1734;
a VL at least 65%, at least 70%, at least 75%, at least 80%, at least 85%, at least 90%, at least 95%, at least 99%, or 100% identical to SEQ ID NO: 1740 and a VH at least 65%, at least 70%, at least 75%, at least 80%, at least 85%, at least 90%, at least 95%, at least 99%, or 100% identical to SEQ ID NO: 1742;
a VL at least 65%, at least 70%, at least 75%, at least 80%, at least 85%, at least 90%, at least 95%, at least 99%, or 100% identical to SEQ ID NO: 1746 and a VH at least 65%, at least 70%, at least 75%, at least 80%, at least 85%, at least 90%, at least 95%, at least 99%, or 100% identical to SEQ ID NO: 1750;
a VL at least 65%, at least 70%, at least 75%, at least 80%, at least 85%, at least 90%, at least 95%, at least 99%, or 100% identical to SEQ ID NO: 1755 and a VH at least 65%, at least 70%, at least 75%, at least 80%, at least 85%, at least 90%, at least 95%, at least 99%, or 100% identical to SEQ ID NO: 1759;
a VL at least 65%, at least 70%, at least 75%, at least 80%, at least 85%, at least 90%, at least 95%, at least 99%, or 100% identical to SEQ ID NO: 1755 and a VH at least 65%, at least 70%, at least 75%, at least 80%, at least 85%, at least 90%, at least 95%, at least 99%, or 100% identical to SEQ ID NO: 1760;
a VL at least 65%, at least 70%, at least 75%, at least 80%, at least 85%, at least 90%, at least 95%, at least 99%, or 100% identical to SEQ ID NO: 1730 and a VH at least 65%, at least 70%, at least 75%, at least 80%, at least 85%, at least 90%, at least 95%, at least 99%, or 100% identical to SEQ ID NO: 1734;
a VL at least 65%, at least 70%, at least 75%, at least 80%, at least 85%, at least 90%, at least 95%, at least 99%, or 100% identical to SEQ ID NO: 1764 and a VH at least 65%, at least 70%, at least 75%, at least 80%, at least 85%, at least 90%, at least 95%, at least 99%, or 100% identical to SEQ ID NO: 1765;
a VL at least 65%, at least 70%, at least 75%, at least 80%, at least 85%, at least 90%, at least 95%, at least 99%, or 100% identical to SEQ ID NO: 1766 and a VH at least 65%, at least 70%, at least 75%, at least 80%, at least 85%, at least 90%, at least 95%, at least 99%, or 100% identical to SEQ ID NO: 1768;
a VL at least 65%, at least 70%, at least 75%, at least 80%, at least 85%, at least 90%, at least 95%, at least 99%, or 100% identical to SEQ ID NO: 1771 and a VH at least 65%, at least 70%, at least 75%, at least 80%, at least 85%, at least 90%, at least 95%, at least 99%, or 100% identical to SEQ ID NO: 1775;
a VL at least 65%, at least 70%, at least 75%, at least 80%, at least 85%, at least 90%, at least 95%, at least 99%, or 100% identical to SEQ ID NO: 1779 and a VH at least 65%, at least 70%, at least 75%, at least 80%, at least 85%, at least 90%, at least 95%, at least 99%, or 100% identical to SEQ ID NO: 1783;
a VL at least 65%, at least 70%, at least 75%, at least 80%, at least 85%, at least 90%, at least 95%, at least 99%, or 100% identical to SEQ ID NO: 1784 and a VH at least 65%, at least 70%, at least 75%, at least 80%, at least 85%, at least 90%, at least 95%, at least 99%, or 100% identical to SEQ ID NO: 1785;
a VL at least 65%, at least 70%, at least 75%, at least 80%, at least 85%, at least 90%, at least 95%, at least 99%, or 100% identical to SEQ ID NO: 1788 and a VH at least 65%, at least 70%, at least 75%, at least 80%, at least 85%, at least 90%, at least 95%, at least 99%, or 100% identical to SEQ ID NO: 1792;
a VL at least 65%, at least 70%, at least 75%, at least 80%, at least 85%, at least 90%, at least 95%, at least 99%, or 100% identical to SEQ ID NO: 1794 and a VH at least 65%, at least 70%, at least 75%, at least 80%, at least 85%, at least 90%, at least 95%, at least 99%, or 100% identical to SEQ ID NO: 1797;
a VL at least 65%, at least 70%, at least 75%, at least 80%, at least 85%, at least 90%, at least 95%, at least 99%, or 100% identical to SEQ ID NO: 1800 and a VH at least 65%, at least 70%, at least 75%, at least 80%, at least 85%, at least 90%, at least 95%, at least 99%, or 100% identical to SEQ ID NO: 1804;
a VL at least 65%, at least 70%, at least 75%, at least 80%, at least 85%, at least 90%, at least 95%, at least 99%, or 100% identical to SEQ ID NO: 1806 and a VH at least 65%, at least 70%, at least 75%, at least 80%, at least 85%, at least 90%, at least 95%, at least 99%, or 100% identical to SEQ ID NO: 1809;
a VL at least 65%, at least 70%, at least 75%, at least 80%, at least 85%, at least 90%, at least 95%, at least 99%, or 100% identical to SEQ ID NO: 1812 and a VH at least 65%, at least 70%, at least 75%, at least 80%, at least 85%, at least 90%, at least 95%, at least 99%, or 100% identical to SEQ ID NO: 1816;
a VL at least 65%, at least 70%, at least 75%, at least 80%, at least 85%, at least 90%, at least 95%, at least 99%, or 100% identical to SEQ ID NO: 1819 and a VH at least 65%, at least 70%, at least 75%, at least 80%, at least 85%, at least 90%, at least 95%, at least 99%, or 100% identical to SEQ ID NO: 1823;
a VL at least 65%, at least 70%, at least 75%, at least 80%, at least 85%, at least 90%, at least 95%, at least 99%, or 100% identical to SEQ ID NO: 1826 and a VH at least 65%, at least 70%, at least 75%, at least 80%, at least 85%, at least 90%, at least 95%, at least 99%, or 100% identical to SEQ ID NO: 1830;
a VL at least 65%, at least 70%, at least 75%, at least 80%, at least 85%, at least 90%, at least 95%, at least 99%, or 100% identical to SEQ ID NO: 1980 and a VH at least 65%, at least 70%, at least 75%, at least 80%, at least 85%, at least 90%, at least 95%, at least 99%, or 100% identical to SEQ ID NO: 1984;
a VL at least 65%, at least 70%, at least 75%, at least 80%, at least 85%, at least 90%, at least 95%, at least 99%, or 100% identical to SEQ ID NO: 1988 and a VH at least 65%, at least 70%, at least 75%, at least 80%, at least 85%, at least 90%, at least 95%, at least 99%, or 100% identical to SEQ ID NO: 1992;
a VL at least 65%, at least 70%, at least 75%, at least 80%, at least 85%, at least 90%, at least 95%, at least 99%, or 100% identical to SEQ ID NO: 1996 and a VH at least 65%, at least 70%, at least 75%, at least 80%, at least 85%, at least 90%, at least 95%, at least 99%, or 100% identical to SEQ ID NO: 2000;
a VL at least 65%, at least 70%, at least 75%, at least 80%, at least 85%, at least 90%, at least 95%, at least 99%, or 100% identical to SEQ ID NO: 2004 and a VH at least 65%, at least 70%, at least 75%, at least 80%, at least 85%, at least 90%, at least 95%, at least 99%, or 100% identical to SEQ ID NO: 2008;
a VL at least 65%, at least 70%, at least 75%, at least 80%, at least 85%, at least 90%, at least 95%, at least 99%, or 100% identical to SEQ ID NO: 1833 and a VH at least 65%, at least 70%, at least 75%, at least 80%, at least 85%, at least 90%, at least 95%, at least 99%, or 100% identical to SEQ ID NO: 1837;
a VL at least 65%, at least 70%, at least 75%, at least 80%, at least 85%, at least 90%, at least 95%, at least 99%, or 100% identical to SEQ ID NO: 1840 and a VH at least 65%, at least 70%, at least 75%, at least 80%, at least 85%, at least 90%, at least 95%, at least 99%, or 100% identical to SEQ ID NO: 1844;
a VL at least 65%, at least 70%, at least 75%, at least 80%, at least 85%, at least 90%, at least 95%, at least 99%, or 100% identical to SEQ ID NO: 1847 and a VH at least 65%, at least 70%, at least 75%, at least 80%, at least 85%, at least 90%, at least 95%, at least 99%, or 100% identical to SEQ ID NO: 1851;
a VL at least 65%, at least 70%, at least 75%, at least 80%, at least 85%, at least 90%, at least 95%, at least 99%, or 100% identical to SEQ ID NO: 1854 and a VH at least 65%, at least 70%, at least 75%, at least 80%, at least 85%, at least 90%, at least 95%, at least 99%, or 100% identical to SEQ ID NO: 1858;
a VL at least 65%, at least 70%, at least 75%, at least 80%, at least 85%, at least 90%, at least 95%, at least 99%, or 100% identical to SEQ ID NO: 1861 and a VH at least 65%, at least 70%, at least 75%, at least 80%, at least 85%, at least 90%, at least 95%, at least 99%, or 100% identical to SEQ ID NO: 1865;
a VL at least 65%, at least 70%, at least 75%, at least 80%, at least 85%, at least 90%, at least 95%, at least 99%, or 100% identical to SEQ ID NO: 1361 and a VH at least 65%, at least 70%, at least 75%, at least 80%, at least 85%, at least 90%, at least 95%, at least 99%, or 100% identical to SEQ ID NO: 1365;
a VL at least 65%, at least 70%, at least 75%, at least 80%, at least 85%, at least 90%, at least 95%, at least 99%, or 100% identical to SEQ ID NO: 1868 and a VH at least 65%, at least 70%, at least 75%, at least 80%, at least 85%, at least 90%, at least 95%, at least 99%, or 100% identical to SEQ ID NO: 1871;
a VL at least 65%, at least 70%, at least 75%, at least 80%, at least 85%, at least 90%, at least 95%, at least 99%, or 100% identical to SEQ ID NO: 1874 and a VH at least 65%, at least 70%, at least 75%, at least 80%, at least 85%, at least 90%, at least 95%, at least 99%, or 100% identical to SEQ ID NO: 1877;
a VL at least 65%, at least 70%, at least 75%, at least 80%, at least 85%, at least 90%, at least 95%, at least 99%, or 100% identical to SEQ ID NO: 1880 and a VH at least 65%, at least 70%, at least 75%, at least 80%, at least 85%, at least 90%, at least 95%, at least 99%, or 100% identical to SEQ ID NO: 1884;
a VL at least 65%, at least 70%, at least 75%, at least 80%, at least 85%, at least 90%, at least 95%, at least 99%, or 100% identical to SEQ ID NO: 1887 and a VH at least 65%, at least 70%, at least 75%, at least 80%, at least 85%, at least 90%, at least 95%, at least 99%, or 100% identical to SEQ ID NO: 1890;
a VL at least 65%, at least 70%, at least 75%, at least 80%, at least 85%, at least 90%, at least 95%, at least 99%, or 100% identical to SEQ ID NO: 1893 and a VH at least 65%, at least 70%, at least 75%, at least 80%, at least 85%, at least 90%, at least 95%, at least 99%, or 100% identical to SEQ ID NO: 1897;
a VL at least 65%, at least 70%, at least 75%, at least 80%, at least 85%, at least 90%, at least 95%, at least 99%, or 100% identical to SEQ ID NO: 1900 and a VH at least 65%, at least 70%, at least 75%, at least 80%, at least 85%, at least 90%, at least 95%, at least 99%, or 100% identical to SEQ ID NO: 1904;
a VL at least 65%, at least 70%, at least 75%, at least 80%, at least 85%, at least 90%, at least 95%, at least 99%, or 100% identical to SEQ ID NO: 1907 and a VH at least 65%, at least 70%, at least 75%, at least 80%, at least 85%, at least 90%, at least 95%, at least 99%, or 100% identical to SEQ ID NO: 1910;
a VL at least 65%, at least 70%, at least 75%, at least 80%, at least 85%, at least 90%, at least 95%, at least 99%, or 100% identical to SEQ ID NO: 1913 and a VH at least 65%, at least 70%, at least 75%, at least 80%, at least 85%, at least 90%, at least 95%, at least 99%, or 100% identical to SEQ ID NO: 1916;
a VL at least 65%, at least 70%, at least 75%, at least 80%, at least 85%, at least 90%, at least 95%, at least 99%, or 100% identical to SEQ ID NO: 1919 and a VH at least 65%, at least 70%, at least 75%, at least 80%, at least 85%, at least 90%, at least 95%, at least 99%, or 100% identical to SEQ ID NO: 1923;
a VL at least 65%, at least 70%, at least 75%, at least 80%, at least 85%, at least 90%, at least 95%, at least 99%, or 100% identical to SEQ ID NO: 1926 and a VH at least 65%, at least 70%, at least 75%, at least 80%, at least 85%, at least 90%, at least 95%, at least 99%, or 100% identical to SEQ ID NO: 1930;
a VL at least 65%, at least 70%, at least 75%, at least 80%, at least 85%, at least 90%, at least 95%, at least 99%, or 100% identical to SEQ ID NO: 1933 and a VH at least 65%, at least 70%, at least 75%, at least 80%, at least 85%, at least 90%, at least 95%, at least 99%, or 100% identical to SEQ ID NO: 1937;
a VL at least 65%, at least 70%, at least 75%, at least 80%, at least 85%, at least 90%, at least 95%, at least 99%, or 100% identical to SEQ ID NO: 1940 and a VH at least 65%, at least 70%, at least 75%, at least 80%, at least 85%, at least 90%, at least 95%, at least 99%, or 100% identical to SEQ ID NO: 1944;
a VL at least 65%, at least 70%, at least 75%, at least 80%, at least 85%, at least 90%, at least 95%, at least 99%, or 100% identical to SEQ ID NO: 1947 and a VH at least 65%, at least 70%, at least 75%, at least 80%, at least 85%, at least 90%, at least 95%, at least 99%, or 100% identical to SEQ ID NO: 1950;
a VL at least 65%, at least 70%, at least 75%, at least 80%, at least 85%, at least 90%, at least 95%, at least 99%, or 100% identical to SEQ ID NO: 1952 and a VH at least 65%, at least 70%, at least 75%, at least 80%, at least 85%, at least 90%, at least 95%, at least 99%, or 100% identical to SEQ ID NO: 1956;
a VL at least 65%, at least 70%, at least 75%, at least 80%, at least 85%, at least 90%, at least 95%, at least 99%, or 100% identical to SEQ ID NO: 1958 and a VH at least 65%, at least 70%, at least 75%, at least 80%, at least 85%, at least 90%, at least 95%, at least 99%, or 100% identical to SEQ ID NO: 1962;
a VL at least 65%, at least 70%, at least 75%, at least 80%, at least 85%, at least 90%, at least 95%, at least 99%, or 100% identical to SEQ ID NO: 1965 and a VH at least 65%, at least 70%, at least 75%, at least 80%, at least 85%, at least 90%, at least 95%, at least 99%, or 100% identical to SEQ ID NO: 1969; or
a VL at least 65%, at least 70%, at least 75%, at least 80%, at least 85%, at least 90%, at least 95%, at least 99%, or 100% identical to SEQ ID NO: 1971 and a VH at least 65%, at least 70%, at least 75%, at least 80%, at least 85%, at least 90%, at least 95%, at least 99%, or 100% identical to SEQ ID NO: 1974.

In some aspects, the antigen binding polypeptide complex or the antibody or antigen binding fragment thereof disclosed herein comprise one or more CL and wherein the one or more CL comprise an amino acid sequence that is at least 65%, at least 70%, at least 75%, at least 80%, at least 85%, at least 90%, at least 95%, at least 99%, or 100% identical to any one of SEQ ID NOs: 374, 637, or 1092-1095. In some aspects, the antigen binding polypeptide complex or the antibody or antigen binding fragment thereof disclosed herein comprise one or more CH1 and wherein the one or more CH1 comprise an amino acid sequence that is at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptide complex or the antibody or antigen binding fragment thereof disclosed herein comprise one or more CH2 and wherein the one or more CH2 comprise an amino acid sequence that is at least 65%, at least 70%, at least 75%, at least 80%, at least 85%, at least 90%, at least 95%, at least 99%, or 100% identical to any one of SEQ ID NO: 1075-1078. In some aspects, the antigen binding polypeptide complex or the antibody or antigen binding fragment thereof disclosed herein comprise one or more CH3 and wherein the one or more CH3 comprise an amino acid sequence that is at least 65%, at least 70%, at least 75%, at least 80%, at least 85%, at least 90%, at least 95%, at least 99%, or 100% identical to any one of SEQ ID NO: 1079-1085.

In some aspects, the antigen binding polypeptide, antigen binding polypeptide complex, polypeptide, antibody or antigen binding fragment thereof disclosed herein have a poly-reactivity score of from 0 to 100. In some aspects, the antigen binding polypeptide, antigen binding polypeptide complex, polypeptide, antibody or antigen binding fragment thereof disclosed herein have a poly-reactivity score of from 0 to 50. In some aspects, the antigen binding polypeptide, antigen binding polypeptide complex, polypeptide, antibody or antigen binding fragment thereof disclosed herein have a poly-reactivity score of from 0 to 20. In some aspects, the antigen binding polypeptide, antigen binding polypeptide complex, polypeptide, antibody or antigen binding fragment thereof disclosed herein have a poly-reactivity score of from 0 to 10. In some aspects, the antigen binding polypeptide, antigen binding polypeptide complex, polypeptide, antibody or antigen binding fragment thereof disclosed herein have a poly-reactivity score of from 0 to 5.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIGS. 1A-1UU show exemplary linear structures of antigen binding polypeptides derived from anti-SARS-CoV-2 antibodies (i.e., B8, E12, E8, A23-58, A19-46, and B1) as indicated. VL: variable light chain; VH variable heavy chain; CL: light chain constant region; CH: heavy chain constant region; and L: optional linker.

FIGS. 2A-2N show exemplary configurations of antigen binding polypeptides derived from anti-SARS-CoV-2 antibodies (i.e., B8, E12, E8, A23-58, A19-46, and B1) as indicated.

FIG. 3 shows a schematic of the targeting of multiple spike protein receptor binding domain (RBD) sites by anti-SARS-CoV-2 neutralizing antibodies from which the VHs and the VLs comprised in the antigen binding polypeptides described herein were derived.

FIG. 4 shows a schematic of the structure of exemplary anti-SARS-CoV-2 antigen-binding polypeptide complexes tested in Example 4 (MX1042, MX1043, MX1069, MX1055, MX1089, and MX1206).

FIG. 5 shows a chart of the pharmacokinetics (PK) determined for each antigen binding polypeptide complex tested in Example 5 (MX1069, MX1043, MX1206, MX1089, and MX1042).

FIG. 6 shows a schematic of the design of the experiments described in Example 6.

FIG. 7 shows a schematic of the structures of MX1069 and MX1089.

FIG. 8 shows a chart of body weight change in animals treated with MX1069 and MX1089.

FIGS. 9A-9B show a chart of Median Tissue Culture Infectious Dose (TCID50) in lungs and nares in animals treated with MX1069 and MX1089.

FIG. 10 shows a schematic of the structures of MX1042 and MX1043.

FIG. 11 shows a schematic of the design of the experiments described in Example 6.

FIG. 12 shows a chart of body weight change in animals treated with MX1042 and MX1043.

FIGS. 13A-13B show a chart of Median Tissue Culture Infectious Dose (TCID50) in lungs and nares in animals treated with MX1042 and MX1043.

FIGS. 14A-14B show a schematic of the structures of additional candidates tested in Example 7 (MX1255, MX1275, MX1413, MX1368, MX1366, and MX1418).

FIG. 15 shows a schematic of the position of the N62 residue in the VH of the E8 monoclonal antibody.

FIG. 16 shows a chart of the pharmacokinetics (PK) of the indicated monoclonal antibodies.

FIG. 17 shows a schematic of the structures of additional candidates and of the control (MX1042) tested in Example 8 (MX1042, MX1275, MX1448, MX1545, MX1069, MX1366, MX1450, and MX1548).

FIGS. 18A-18B show a chart of the pharmacokinetics (PK) values of the tested antigen binding polypeptide complexes as indicated (MX1042, MX1275, MX1448, MX1545, MX1069, MX1366, MX1450, and MX1548).

FIG. 19 shows a gel electrophoresis of restriction enzyme digestion of MD1186.

FIG. 20 shows a gel electrophoresis of MD1186 plasmid linearized with NotI.

FIGS. 21A-21B show a gel electrophoresis of the mRNA in vitro transcribed from MD1186 plasmid linearized with NotI.

FIG. 22 shows a Western Blot of in vitro produced MX828 after protein A purification.

FIG. 23 shows a schematic of the structure of MX828.

FIG. 24 shows a schematic of the structures of additional candidates tested for in vitro production (MX1545, MX1450/MX1453, MX1548, MX1468, and MX1549).

FIG. 25 shows a gel electrophoresis of in vitro transcribed mRNA encoding MX1448/MX1451 and MX1450/MX1453.

FIG. 26 shows a capillary gel electrophoresis by Fragment Analyzer of in vitro transcribed mRNA encoding MX1448/MX1451 and MX1450/MX1453.

FIG. 27 shows a Western Blot analysis of the produced MX1448/MX1451 and MX1450/MX1453.

FIG. 28 shows a schematic of the structures of MX1451 and MX1453.

FIG. 29 shows a schematic of the structures of MX1548, MX1846, and MX1847.

FIGS. 30A-30B show size exclusion chromatography profiles of MX1846 and MX1847.

FIG. 31 shows a schematic of the structures of MX1450, MX1866, and MX1867.

FIGS. 32A-32B show size exclusion chromatography profiles of MX1866 and MX1867.

FIG. 33 shows a schematic of the structures of MX1549, MX1849, and MX1850.

FIG. 34 shows a schematic of the structures of MX1468, MX1868, and MX1869.

FIGS. 35A-35B show size exclusion chromatography profiles of MX1868 and MX1869.

FIGS. 36A-36E show HPLC-analytical SEC profiles of MX1846, MX1847, MX1866, MX1867, and MX1548.

FIGS. 37A-37E show HIC profiles of MX1846, MX1847, MX1866, MX1867, and MX1548.

FIG. 38 shows a schematic of the structures of MX1870, MX1871, MX1872, MX1873, MX1874, MX1875, MX1876, MX1877, MX1878, MX1879, MX1880, and MX1881.

FIGS. 39A-39L show SEC profiles of MX1870, MX1871, MX1872, MX1873, MX1874, MX1875, MX1876, MX1877, MX1878, MX1879, MX1880, and MX1881.

FIG. 40 shows a schematic of the structures of MX1870, MX1871, MX1872, MX1873, MX1874, MX1875, MX1876, MX1877, MX1878, MX1879, MX1880, MX1881, MX1548, MX1846, MX1847, and MX2005. The TM/YTE mutations occur in the hinge region between CH1 and CH2, and in CH2 and CH3, but not in CL or CH1, CL/CH1 are labeled as TM/YTE in the figure for ease of description.

FIG. 41 shows a schematic of the structures of MX1846, MX1846 with B2. MX 1880, and MX1880 with B2.

FIG. 42 shows a schematic of the structures of MX1846 and MX2005. The TM/YTE mutations occur in the hinge region between CH1 and CH2, and in CH2 and CH3, but not in CL or CH1, CL/CH1 are labeled as TM/YTE in the figure for case of description.

FIG. 43 shows a schematic of the structures of MX2005, MX2167, MX2169, MX2241, MX2148, MX2168, MX2170, MX2242, MX2183, MX2179, MX2180, and MX2269. The TM/YTE mutations occur in the hinge region between CH1 and CH2, and in CH2 and CH3, but not in CL or CH1, CL/CH1 are labeled as TM/YTE in the figure for ease of description.

FIGS. 44A-44J show SEC profiles of MX2167, MX2169, MX2168, MX2170, MX2171, MX2172, MX2148, MX2183, MX2179, and MX2180.

FIG. 45 shows a schematic of the structures of MX1846, MX2005, MX2167, MX2169, MX2148, MX2168, MX2170, MX2171, MX2172, MX2179, MX2180, and MX2183. The TM/YTE mutations occur in the hinge region between CH1 and CH2, and in CH2 and CH3, but not in CL or CH1, CL/CH1 are labeled as TM/YTE in the figure for ease of description.

FIGS. 46A-46B show results of a hamster challenge experiment conducted with MX2005, MX1880 and MX2148.

FIG. 47 shows a schematic of the structures of MX1548, MX1846, MX2005, and MX2059.

FIG. 48 shows representative sequence alignments between Fc domain of TM/YTE, LALAPA-LA and LA.

FIGS. 49A-49B show SEC profiles of MX1846 and MX2005.

FIG. 50 show results of an experiment in FcRn-Fc mice conducted with MX1846 and MX2005.

FIGS. 51A-51B show SEC profiles of MX1846 and MX2059.

FIG. 52 shows schematics of the linear structure of the antigen binding polypeptides comprised in MX1545, MX1733, MX1734, and MX1735.

FIG. 53 shows schematics of the structures of MX1545, MX1733, MX1734, and MX1735.

FIG. 54 shows a schematic of the AlphaFold2 prediction model of the linkers comprised in MX1545, MX1733, MX1734, and MX1735.

FIG. 55 shows SEC profiles of MX1545, MX1733, MX1734, and MX1735.

FIG. 56 shows schematics of the linear structure of the antigen binding polypeptides comprised in MX1545, MX1781, MX1782, and MX1783.

FIG. 57 shows schematics of the structures of MX1545, MX1781, MX1782, and MX1783.

FIG. 58 shows SEC profiles of MX1545, MX1781, MX1782, and MX1783.

FIG. 59 shows schematics of the linear structure of the antigen binding polypeptides comprised in MX1546, MX1570, MX1571, and MX1572.

FIG. 60 shows schematics of the structures of MX1546, MX1570, MX1571, and MX1572.

FIG. 61 shows a schematic of the AlphaFold2 prediction model of the linkers comprised in MX1546, MX1570, MX1571, and MX1572.

FIG. 62 shows SEC profiles of MX1546, MX1570, MX1571, and MX1572.

FIG. 63 shows schematics of the linear structure of the antigen binding polypeptides comprised in MX1544, MX1567, MX1568, and MX1569.

FIG. 64 shows schematics of the structures of MX1544, MX1567, MX1568, and MX1569.

FIG. 65 shows a schematic of the AlphaFold2 prediction model of the linkers comprised in MX1567 and MX1569.

FIG. 66 shows SEC profiles of MX1544, MX1567, MX1568, and MX1569.

FIGS. 67A-67B show schematics of the binding epitope of A18-448 on the spike protein receptor binding domain (RBD).

FIG. 68 shows a schematic of the structure of MX2301. The TM/YTE mutations occur in the hinge region between CH1 and CH2, and in CH2 and CH3, but not in CL or CH1, CL/CH1 are labeled as TM/YTE in the figure for ease of description.

FIG. 69 shows schematics of the structures of MX2299 and MX2265, B2 dGly: B2 comprising the HC-N89Q modification. The TM/YTE mutations occur in the hinge region between CH1 and CH2, and in CH2 and CH3, but not in CL or CH1, CL/CH1 are labeled as TM/YTE in the figure for ease of description.

FIG. 70 shows results of an experiment conducted to investigate the mechanism of action of MX2148, MX2148 blocks ACE2 binding on the SARS-CoV-2 spike protein to prevent viral entry.

FIGS. 71A-71H show results of an experiment conducted to measure the Fc-γ function of MX1880 and MX2148. The Fc mutations of MX2148 eliminated its Fc-γ function.

FIG. 72 shows schematics of the structures of monovalent and bivalent antigen binding complexes comprising A18-448 or B2.

FIGS. 73A-73B show the SEC profiles of MX2301 and MX2299, respectively.

FIG. 74 shows the SEC profile of MX2265.

FIG. 75 shows schematics of the structures of MX2242 and MX2241, B2 dGly: B2 comprising the HC-N89Q modification: G11 dGly: G11 comprising the HC-N116Q modification. The TM/YTE mutations occur in the hinge region between CH1 and CH2, and in CH2 and CH3, but not in CL or CH1. CL/CH1 are labeled as TM/YTE in the figure for ease of description.

FIG. 76 shows the SEC profile of MX2242.

FIGS. 77A-77B show the SEC profiles of MX2241 and MX2242, respectively.

FIG. 78 shows a Western Blot analysis of produced MX2241 and MX2242.

FIG. 79 shows a schematic of the structures of MX2263, MX2264, MX2265, and MX2266, B2 dGly: B2 comprising the HC-N89Q modification. The TM/YTE mutations occur in the hinge region between CH1 and CH2, and in CH2 and CH3, but not in CL or CH1, CL/CH1 are labeled as TM/YTE in the figure for ease of description.

FIGS. 80A-80E show SEC profiles of MX2263, MX2264, MX2265, MX2266, and MX2269, respectively.

FIG. 81 shows a Western Blot analysis of produced MX2263, MX2264, MX2265, MX2266, and MX2269.

FIG. 82 shows the spike amino acid differences in KP.2, KP.2.3, LB.1, KP.3, and KP.3.1.1, with respect to WA1.

FIGS. 83A-83D show results of a pK experiment in FcRn-Fc mice conducted with MX2567, MX2301, MX2242, and MX2265.

FIGS. 84A-84C show schematics of the structures of MX2674, MX2673, MX2672, MX2671, and MX2670, B2 dGly: B2 comprising the HC-N89Q modification. The TM/YTE mutations occur in the hinge region between CH1 and CH2, and in CH2 and CH3, but not in CL or CH1, CL/CH1 are labeled as TM/YTE in the figure for ease of description.

FIGS. 85A-85B show a Western Blot analysis of produced MX2676, MX2675, MX2674, MX2673, MX2672, MX2671, and MX2670.

FIGS. 86A-86G show the SEC profiles of MX2676, MX2675, MX2674, MX2673, MX2672, MX2671, and MX2670.

FIGS. 87A-87B show schematics of the structures of MX2464, MX2463, MX2462, MX2477, MX2476, MX2478, MX2479, and MX2475. The TM/YTE mutations occur in the hinge region between CH1 and CH2, and in CH2 and CH3, but not in CL or CH1, CL/CH1 are labeled as TM/YTE in the figure for ease of description.

FIGS. 88A-88F show the SEC profiles of MX2462, MX2463, MX2464, MX2475, MX2477, and MX2478, respectively.

FIG. 89 shows a Western Blot analysis of produced MX2462, MX2463, MX2464, MX2475, MX2477, and MX2478.

FIG. 90 shows schematics of the structures of MX2384, MX2270, and MX1091.

FIG. 91 shows generic schematics of monovalent, bivalent, and tetravalent antigen binding complexes.

FIG. 92 shows the results of a luciferase assay conducted in Raji B cells to measure the Antibody-Dependent Enhancement (ADE) of MX2148. No ADE was observed with MX2148, MW05: positive control (a monoclonal antibody that binds to SARS-CoV-2 spike protein, has neutralizing activity, and also exhibits ADE effector function).

FIG. 93 shows the results of an assay conducted to evaluate the rate of in vitro resistance acquisition of SARS-Cov-2 virus in presence of MX2148, of E12, G11, E8, A18-448, and of a mixture of E12, G11, E8, and A18-448.

FIG. 94 shows results of a pK experiment in FcRn-Fc mice conducted with MX2148.

FIG. 95 shows the experimental design of the Syrian Hamster Challenge described in Example 18.

FIGS. 96A-96B show the results obtained in lung (FIG. 96A) and nare (FIG. 96B) in the Syrian Hamster Challenge shown in FIG. 95.

FIGS. 97A-97D show cryo-EM structure of Fab binding sites of E12 (FIG. 97A), G11 (FIG. 97B), E8 (FIG. 97C), and A18.448 (FIG. 97 D) on the RBD, FIG. 97A: cryo-EM structures of XBB spike protein in complex with Fab E12 (map at 3.4 Å resolution). FIG. 97B: cryo-EM structure of XB.1.5 spike in complex with Fab G11 (map at 3.43 Å resolution). FIG. 97C: cryo-EM structures of XBB spike protein in complex with Fab E8 (map at 3.4 Å resolution). FIG. 97D: cryo-EM structure of EG.5 spike in complex with Fab A18.448 (Map at 3.59 Å resolution).

FIG. 98 shows a schematic of the rcVSV neutralization assay described in Example 18.

FIG. 100 shows a schematic of the rcVSV selection described in Example 18.

FIG. 101 shows the results of the rcVSV selection described in Example 18.

FIG. 102 shows a schematic of the A18-448 Fab binding epitope on the surface of the receptor binding domain of SARS-CoV-2 spike protein. The epitope mapping is based on the structural data from cryo-EM structure of AG.5 spike protein in complex with A18-448 Fab (Map at 3.59 Å resolution). G5 Fab binding epitope on the surface of the receptor binding domain of SARS-CoV-2 spike protein is also shown. The epitope mapping is based on the structural data from cryo-EM structure of SARS-CoV-1 spike protein in complex with G5 Fab. Receptor binding domain (RBD) conservation is calculated based on analysis of SARS-CoV-2 GISAID.

FIG. 103 shows a schematic of the structures of MX2242, MX2265, and MX2301.

FIGS. 104A-104C show the results of a purity assessment by HPLC aSEC for MX2242, MX2265, and MX2301, as indicated.

FIGS. 105A-105C show the results of a purity assessment by DLS for MX2242, MX2265, and MX2301, as indicated.

FIGS. 106A-106C show the results of a surface hydrophobicity analysis by aHIC for MX2242, MX2265, and MX2301, as indicated.

FIGS. 107A-107C show the results of a binding characterization by BLI for MX2242, MX2265, and MX2301, as indicated.

FIGS. 108A-108C show the results of a thermostability analysis by DSC for MX2242, MX2265, and MX2301, as indicated.

FIGS. 109A-109C show the results of a cIEF analysis of MX2242, MX2265, and MX2301, as indicated.

FIGS. 110A-110C show the results of a solubility at high concentration analysis by aSEC for MX2242, MX2265, and MX2301, as indicated.

FIGS. 111A-111C show the results of a solubility at high concentration analysis by DLS for MX2242, MX2265, and MX2301, as indicated.

FIGS. 112A-112C show the results of a HPLC-aSEC analysis under 5 cycles of Freeze and Thaw for MX2242, MX2265, and MX2301, as indicated.

FIGS. 113A-113C show the results of a DLS analysis after 5 cycles of Freeze and Thaw for MX2242, MX2265, and MX2301, as indicated.

FIGS. 114A-114C show the results of aSEC analysis under pH stress (pH 3.5 and pH 9.0) for MX2242, MX2265, and MX2301, as indicated.

FIGS. 115A-115C show the results of a potency analysis after pH 3.5 and pH 9.0 stress for MX2242, MX2265, and MX2301, as indicated.

FIGS. 116A-116C show the results of aSEC analysis after incubation at 40° C., for up to 2 weeks for MX2242, MX2265, and MX2301, as indicated.

FIGS. 117A-117C show the results of a binding response to COVID WT (wild type) RBD by BLI for MX2242, MX2265, and MX2301, as indicated.

FIGS. 118A-118C show the results of aSEC analysis under PBS incubation at 37° C., for up to 7 days for MX2242, MX2265, and MX2301, as indicated.

FIGS. 119A-119C show the results of a potency assay (binding to COVID RBD WT) by BLI after serum (0.8×) or PBS incubation at 37° C., for 0-7 days, for MX2242, MX2265, and MX2301, as indicated.

FIGS. 120A-120B show the results of a CE-SDS analysis of MX2242, MX2265, and MX2301 under different stress conditions: 40° C., for 1 week. 40° C., for 2 weeks, pH 3.5, pH 9.0, freeze and thaw, and high concentration.

FIG. 121 shows the results of a poly-reactivity by ELISA panel assay for MX2242, MX2265, and MX2301, and control molecules.

FIG. 122 shows the results of a self-interaction by AC-SINS (Affinity-Capture Self-Interacting Nanoparticle Spectroscopy) assay for MX2242, MX2265, and MX2301, and control molecules.

FIGS. 123A-123F show the results of a LC/MS analysis of MX2242 (samples treated by PNGase F).

FIGS. 124A-124D show the results of a LC/MS analysis of MX2265 (samples treated by PNGase F and TCEP).

FIGS. 125A-125F show the results of a LC/MS analysis of MX2265 (samples treated by Fabdello enzyme and TCEP).

FIGS. 126A-126D show the results of a LC/MS analysis of MX2265 (untreated samples).

FIGS. 127A-127D show the results of a LC/MS analysis of MX2301 (untreated samples).

FIGS. 128A-128D show the results of a LC/MS analysis of MX2301 (samples treated by Fabdello enzyme).

FIGS. 129A-129E show the results of a LC/MS analysis of MX2301.

FIGS. 130A-130C show the results of a DLS (intensity) analysis for MX2242, MX2265, and MX2301, as indicated.

### DETAILED DESCRIPTION OF THE INVENTION

Provided herein are antigen binding polypeptides and polypeptide complexes having improved features. In some aspects, the antigen binding polypeptides and polypeptide complexes have broad reactivity against SARS-CoV-2 variants of concern (VOCs), high neutralization potency, and/or resistance to SARS-CoV-2 escape. In some aspects, the antigen binding polypeptides and polypeptide complexes provided herein are easier to manufacture and administer than current therapeutic antibodies, and antibody cocktails. In some aspects, the antigen binding polypeptides and polypeptide complexes treat and/or prevent SARS-CoV-2 infection and/or COVID-19. In some aspects, the antigen binding polypeptides and polypeptide complexes treat and/or prevent SARS-CoV-2 infection and/or COVID-19 in immunocompromised individuals who cannot mount an effective immune response through vaccinations.

Various terms relating to aspects of the disclosure are used throughout the specification and claims. Such terms are to be given their ordinary meaning in the art, unless otherwise indicated. Other specifically defined terms are to be construed in a manner consistent with the definitions provided herein.

Provided herein are antigen binding polypeptides and polypeptide complexes having improved features. In some aspects, the antigen binding polypeptides and polypeptide complexes have reactivity against one or more antigens and/or high binding affinity to one or more antigens. In some aspects, the antigens are viral antigens that are not sarbecovirus antigens, bacterial antigens, parasite antigens, tumor-associated antigens, or antigens associated with pathologies other than infectious diseases and cancers/tumors. In some aspects, the antigen binding polypeptides and polypeptide complexes provided herein are easier to manufacture and administer than current therapeutic antibodies, and antibody cocktails. In some aspects, the antigen binding polypeptides and polypeptide complexes treat and/or prevent a pathology. In some aspects, the pathology is an infectious disease, such as a viral infection which is not a sarbecovirus infection, a bacterial infection, or a parasite infection: a cancer/tumor; or a pathology other than an infectious disease and a cancer/tumor. In some aspects, the antigen binding polypeptides and polypeptide complexes treat and/or prevent a pathology in immunocompromised individuals who cannot mount an effective immune response through vaccinations.

Various terms relating to aspects of the disclosure are used throughout the specification and claims. Such terms are to be given their ordinary meaning in the art, unless otherwise indicated. Other specifically defined terms are to be construed in a manner consistent with the definitions provided herein.

### Definitions

As used herein the term “SARS-CoV-2” refers to “severe acute respiratory syndrome coronavirus 2.” SARS-CoV-2 is the strain of coronavirus that causes coronavirus disease 2019 (COVID-19).

As used herein, the term “SARS-CoV-2 virus” includes the SARS-CoV-2 virus, virion or fragment thereof.

As used herein the term “COVID-19” refers to “coronavirus disease 2019.” COVID-19 is caused by the SARS-CoV-2 virus. Symptoms of COVID-19 are variable, but often include fever, cough, headache, fatigue, breathing difficulties, loss of smell, and loss of taste. Symptoms may begin one to fourteen days after exposure to the virus; however, at least a third of people who are infected do not develop noticeable symptoms. Of those people who develop symptoms noticeable enough to be classed as patients, most develop mild to moderate symptoms (up to mild pneumonia), with approximately 15% developing severe symptoms (dyspnea, hypoxia, or more than 50% lung involvement on imaging), and approximately 5% suffering critical symptoms (respiratory failure, shock, or multiorgan dysfunction). Some people continue to experience a range of effects for months after recovery (“long COVID”).

As used herein, the term “SARS-CoV-2 protein” refers to any structural or non-structural protein found in the SARS-CoV-2 virus. Structural proteins of the SARS-CoV-2 virus include, but are not limited to, the spike(S), nucleocapsid (N), membrane (M), and envelope (E) proteins. Non-structural proteins (NSPs) of the SARS-CoV-2 virus include, but are not limited to, accessory and replicase proteins such as NSP1-NSP16. The structural and functional properties of these proteins have been characterized. See, e.g., Satarker et al., Arch. Med. Res. 51 (6): 482-491, 2020; and Kakavandi et al., Cell Commun. Signal. 21:110, 2023.

As used herein, the terms “SARS-CoV-2 spike protein” and “spike protein” refer to the largest of the four major structural proteins found in the SARS-CoV-2 virus. The spike protein assembles into trimers that form large structures, called spikes or peplomers, that project from the surface of the SARS-CoV-2 virion and mediate entry of the virion into host cells. The SARS-CoV-2 spike protein is also called “S protein.” “spike (S) glycoprotein” and “E2.” The structural and functional properties of this protein have been characterized. See, e.g., Huang et al., Acta Pharmacol. Sin. 41: 1141-1149, 2020; Wu et al., Nature 579 (7798): 265-269, 2020; and Gene ID: 43740568.

As used herein, the term “antigen binding polypeptide” refers to a polypeptide having the ability to specifically bind to one or more antigens. An “antigen” is any substance (e.g., a molecule, such as a protein) that is recognized by, interacts with, and/or is capable of binding with a component of the immune system (e.g., antibody or antigen binding fragments thereof) and/or that is recognized by, interacts with, and/or is capable of binding with a non-naturally occurring antigen binding polypeptide. An antigen can be a substance (e.g., a molecule, such as a protein) that is (or is recognized by an organism as being) foreign to an organism. “Infectious disease antigens” can be, for example, viral antigens (e.g., sarbecovirus antigens, such as SARS-CoV-2 spike proteins, or epitopes thereof), bacterial antigens, or parasite antigens, which are antigens associated with or derived from viruses, bacteria, or parasites, respectively. Viral antigens can be sarbecovirus antigens or non-sarbecovirus antigens (i.e., antigens associated with or derived from viruses that are not sarbecoviruses. Antigens can also be “tumor-associated antigens” or “TAA.” TAA are molecules that are found only on cancer/tumor cells and not on non-cancer/tumor cells, or molecules that are found in different amounts on cancer/tumor cells with respect to non-cancer/tumor cells, or molecules that can be targeted by (i.e., recognized by, interact with, and/or bound by) antigen binding polypeptides for treating or preventing a cancer/tumor. In addition to infectious disease and cancer/tumor, antigens can also be associated with other pathologies. Such antigens can be found on cells of an organism affected with the pathology, or can be found in different amounts on cells affected with the pathology with respect to cells non affected with the pathology, or can be targeted by (i.e., recognized by, interact with, and/or bound by) antigen binding polypeptides for treating or preventing the pathology. Antigens that can be associated with pathologies other than infectious disease and cancer/tumor are herein referred to also as “other-pathology antigens.” A “pathology” refers to any deviations from the healthy state, and encompasses a “disease.” a “disorder.” a “syndrome.” a “condition.” or any combination thereof. It is to be understood that the descriptions provided above are not mutually exclusive. For example, a single antigen can be a TAA and also a viral antigen; a single antigen can be a TAA and also a bacterial antigen; a single antigen can be associated with more than one pathology; etc. The antigen binding polypeptides disclosed herein have the ability to specifically bind to one or more antigens, including viral antigens (e.g., sarbecovirus antigens, such as SARS-CoV-2 spike proteins, or epitopes thereof; or viral antigens that are not sarbecovirus antigens), bacterial antigens, parasite antigens, tumor-associated antigens, or antigens associated with pathologies other than infectious disease and cancer/tumor.

As used herein, the term “antigen binding polypeptide complex” refers to an aggregate of antigen binding polypeptides. Such aggregates can comprise two or more (e.g., three, four, five, or six) antigen binding polypeptides. Antigen binding polypeptides can be covalently or non-covalently bound to one another to form an antigen binding polypeptide complex. An antigen binding polypeptide complex, includes, but is not limited to, an antibody or antigen binding fragment thereof.

A number of sequences of polypeptides capable of binding SARS-CoV-2 spike proteins antigens (i.e., SARS-CoV-2 spike proteins, or epitopes thereof) are described in the published literature, for example, in U.S. Appl. No. 63/147,419. These sequences include, for example, a complementarity determining region (CDR), heavy chain variable region (VH), light chain variable region (VL), heavy chain (HC) and light chain (LC) sequences of anti-SARS-CoV-2 antibodies or antigen binding fragments thereof. A number of sequences of polypeptides capable of binding one or more antigens, including viral antigens that are not sarbecovirus antigens, bacterial antigens, parasite antigens, tumor-associated antigens, or antigens associated with pathologies other than infectious disease and cancer/tumor are described in the published literature. These sequences include, for example, a complementarity determining region (CDR), heavy chain variable region (VH), light chain variable region (VL), heavy chain (HC) and light chain (LC) sequences of antibodies or antigen binding fragments thereof.

The term “antibody” includes, without limitation, immunoglobulins which bind specifically to an antigen. An antibody comprises two heavy chains (HC) and two light chains (LC) interconnected by covalent bonds (e.g., disulfide bonds). Each HC comprises a heavy chain variable region (VH) and a heavy chain constant region; the heavy chain constant region comprises three constant domains (CH1, CH2 and CH3); each light chain comprises a light chain variable region (VL) and a light chain constant region or domain (CL). The VH and VL comprise regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The VH and the VL comprise binding domains that interact with the antigen. The constant regions of the antibodies may mediate the binding of the immunoglobulin to host tissues or factors, including various cells of the immune system (e.g., effector cells) and the first component (C1q) of the classical complement system. The region of the antibody comprising the VH and the VL is also referred to as Fv. The region of the antibody comprising the VH, the VL, the CH1, and the CL, is also referred to as Fab. The region of the antibody comprising the CH2 and the CH3 is also referred to as Fc. A heavy chain may have the C-terminal lysine. Unless specified otherwise herein, the amino acids in the variable regions are numbered using the Kabat numbering system and those in the constant regions are numbered using the EU system.

An “antigen binding fragment” of an antibody refers to one or more portions of an antibody that retain the ability to specifically bind to the antigen bound by the whole antibody. It has been shown that the antigen binding function of an antibody can be performed by portions of a full-length antibody. An antigen binding fragment can contain the antigen binding regions of an intact antibody (e.g., the complementarity determining regions (CDRs)). Examples of antigen binding fragments of antibodies include, but are not limited to. Fab, Fab′, F(ab′)₂, and Fv fragments, linear antibodies, and single chain antibodies. An antigen binding fragment of an antibody can be derived from any animal species, such as rodents (e.g., mouse, rat, or hamster) and humans or can be artificially produced.

Furthermore, although the two domains of the Fv fragment. VL and VH, are coded for by separate genes, they can be joined, using recombinant methods (e.g., by a synthetic linker) such that the VL and the VH are comprised in a single amino acid chain (known as single chain Fv (scFv): sec, e.g., Bird et al. (1988) Science 242:423-426; and Huston et al. (1988) Proc. Natl. Acad. Sci. USA 85:5879-5883), scFvs are also intended to be encompassed within the term “antigen-binding fragment”.

Antigen binding fragments are obtained using conventional techniques known to those with skill in the art, and the fragments screened for utility in the same manner as are intact antibodies. Antigen binding fragments can be produced by recombinant DNA techniques, or by enzymatic or chemical cleavage of intact immunoglobulins.

An antibody or an antigen binding fragment can be modified compared to a naturally occurring antibody or antigen binding fragment (e.g., by amino acid deletion, insertion, or substitution, or by conjugation to a non-antibody moiety). For example, an antibody or antigen binding fragment thereof can include one or more variant amino acids (compared to a naturally occurring antibody or antigen binding fragment thereof) which change a property (e.g., a functional property) of the antibody or antigen binding fragment thereof. For example, several such alterations are known in the art which affect, e.g., half-life, effector function, and/or immune responses to the antibody or antigen binding fragment thereof in an organism (e.g., a human patient). The term antibody or antigen binding fragment thereof also includes artificial polypeptide constructs which comprise at least one antibody-derived antigen binding site.

As used herein, the term “variable region” typically refers to a portion of an antibody, generally, a portion of a light or heavy chain, typically about the amino-terminal 110 to 120 amino acids, or 110 to 125 amino acids in the mature heavy chain and about 90 to 115 amino acids in the mature light chain, which differ extensively in sequence among antibodies and determine the binding specificity of a particular antibody (or antigen binding fragment thereof) for a particular antigen. The sequence variability is concentrated the CDRs, while the more conserved regions form the FRs. Without wishing to be bound by any particular mechanism or theory, it is believed that the CDRs of the light and heavy chains are primarily responsible for the interaction and specificity of an antibody (or antigen binding fragment thereof) with antigen. In some aspects, the variable region is a mammalian variable region, e.g., a human, mouse or rabbit variable region. In some aspects, the variable region comprises rodent or murine CDRs and human FRs. In some aspects, the variable region is a primate (e.g., non-human primate) variable region. In some aspects, the variable region comprises rodent or murine CDRs and primate (e.g., non-human primate) FRs.

The terms “complementarity determining region” or “CDR”, as used herein, refer to each of the regions of an antibody variable domain which are hypervariable in sequence and/or form structurally defined loops (hypervariable loops) and/or contain the antigen-contacting residues. Antibodies (or antigen binding fragments thereof) can comprise six CDRs, e.g., three in the VH and three in the VL.

The terms “VL”. “VL region.” and “VL domain” are used herein interchangeably to refer to the light chain variable region of an antigen binding polypeptide, antigen binding polypeptide complex, antibody or antigen binding fragment thereof. In some aspects, a VL region is referred to herein as VL1 to denote a first light chain variable region. VL2 to denote a second light chain variable region. VL3 to denote a third light chain variable region, and VL4 to denote a fourth light chain variable region. An enumerated VL region (e.g., VL1) can have the same or different antigen binding properties and/or the same or different sequence as another enumerated VL region (e.g., VL2).

The terms “VH”. “VH region.” and “VH domain” are used herein interchangeably to refer to the heavy chain variable region of an antigen binding polypeptide, antigen binding polypeptide complex, antibody or antigen binding fragment thereof. In some aspects, a VH region is referred to herein as VH1 to denote a first heavy chain variable region. VH2 to denote a second heavy chain variable region, and VH3 to denote a third heavy chain variable region. An enumerated VH region (e.g., VH1) can have the same or different antigen binding properties and/or the same or different sequence as another enumerated VH region (e.g., VH2).

As used herein. “Kabat numbering” and like terms are recognized in the art and refer to a system of numbering amino acid residues in the heavy and light chain variable regions of an antibody or antigen binding fragment thereof. In some aspects, CDRs can be determined according to the Kabat numbering system (sec, e.g., Kabat E A & Wu T T (1971) Ann NY Acad Sci 190:382-391 and Kabat E A et al., (1991) Sequences of Proteins of Immunological Interest. Fifth Edition. U.S. Department of Health and Human Services. NIH Publication No. 91-3242). Using the Kabat numbering system. CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system. CDRs within an antibody light chain molecule are typically present at amino acid positions 24 to 34 (CDR1), amino acid positions 50 to 56 (CDR2), and amino acid positions 89 to 97 (CDR3).

As used herein, the terms “constant region” or “constant domain” are used interchangeably to refer to a portion of an antigen binding polypeptide, antigen binding polypeptide complex, antibody or antigen binding fragment thereof, e.g., a carboxyl terminal portion of a light and/or heavy chain which is not directly involved in binding of an antibody (or antigen binding fragment thereof) to an antigen but which can exhibit various effector functions. The constant region generally has a more conserved amino acid sequence relative to a variable region. In some aspects, an antigen binding polypeptide, antigen binding polypeptide complex, antibody or antigen binding fragment thereof comprises a constant region or portion thereof that is sufficient for antibody-dependent cell-mediated cytotoxicity (ADCC), antibody-dependent cellular phagocytosis (ADCP), and complement-dependent cytotoxicity (CDC). A constant region includes, but is not limited to, a light chain constant region or domain (CL) or heavy chain constant region or domain (CH1, CH2, CH3).

As used herein, the terms “fragment crystallizable region.” “Fc region.” or “Fc domain” are used interchangeably to refer to the tail region of an antibody that can interacts with cell surface receptors called Fc receptors and some proteins of the complement system. Fc regions typically comprise CH2 and CH3 regions, and, optionally, an immunoglobulin hinge. The immunoglobulin hinge typically connects the CH2 and CH3 regions to the CH1 region.

As used herein, the terms “immunoglobulin hinge.” “hinge.” “hinge domain” or “hinge region” are used interchangeably to refer to a stretch of heavy chains between the Fab and Fc portions of an antigen binding polypeptide, antigen binding polypeptide complex, antibody or antigen binding fragment thereof. A hinge provides structure, position and flexibility, which assist with normal functioning of antibodies (e.g., for crosslinking two antigens or binding two antigenic determinants on the same antigen molecule). An immunoglobulin hinge is divided into upper, middle and lower hinge regions that can be separated based on structural and/or genetic components. An immunoglobulin hinge of the invention can contain one, two or all three of these regions. Structurally, the upper hinge region stretches from the C terminal end of CH1 to the first hinge disulfide bond. The middle hinge region stretches from the first cysteine to the last cysteine in the hinge. The lower hinge region extends from the last cysteine to a glycine of CH2. The cysteines present in the hinge form interchain disulfide bonds that link the immunoglobulin monomers.

As used herein, the term “heavy chain” refers to a portion of an antigen binding polypeptide, antigen binding polypeptide complex, antibody or antigen binding fragment thereof typically composed of a heavy chain variable region (VH), a heavy chain constant region 1 (CH1), a heavy chain constant region 2 (CH2), and a heavy chain constant region 3 (CH3). A typical antibody is composed of two heavy chains and two light chains. When used in reference to an antibody, a heavy chain can refer to any distinct type, e.g., alpha (α), delta (δ), epsilon (ε), gamma (γ), and mu (μ), based on the amino acid sequence of the constant region, which gives rise to IgA, IgD, IgE, IgG, and IgM classes of antibodies, respectively, including subclasses of IgG, e.g., IgG1, IgG2, IgG3, and IgG4. Heavy chain amino acid sequences are known in the art. In some aspects, the heavy chain is a human heavy chain.

As used herein, the term “light chain” refers to a portion of an antigen binding polypeptide, antigen binding polypeptide complex, antibody or antigen binding fragment thereof typically composed of a light chain variable region (VL) and a light chain constant region (CL). A typical antibody is composed of two light chains and two heavy chains. When used in reference to an antibody, a light chain can refer to any distinct type, e.g., kappa (κ) or lambda (λ), based on the amino acid sequence of the constant region. Light chain amino acid sequences are known in the art. In some aspects, the light chain is a human light chain.

The term “antibody” includes, by way of example, monoclonal and polyclonal antibodies: chimeric and humanized antibodies: human or non-human antibodies: wholly synthetic antibodies; and single chain antibodies. A non-human antibody can be humanized by recombinant methods to reduce its immunogenicity in humans. Antigen binding fragments of an antibody can also be monoclonal and polyclonal, chimeric and humanized, human or non-human, wholly synthetic, and single chain. A non-human antigen binding fragments of an antibody can be humanized by recombinant methods to reduce its immunogenicity in humans.

The term “monoclonal antibody.” or antigen binding fragment thereof, as used herein, refers to an antibody or a population of antibodies, or antigen binding fragments thereof, that are produced by a single clone of B-cells and bind to the same epitope. In contrast, the term “polyclonal antibody.” or antigen binding fragment thereof, refers to a population of antibodies, or antigen binding fragment thereof, that are produced by different B-cells and can bind to the same or different epitopes of the same antigen.

The term “chimeric antibody.” or antigen binding fragment thereof, refers to an antibody or antigen binding fragments thereof wherein the amino acid sequence is derived from two or more species. Typically, the variable region of both light and heavy chains corresponds to the variable region of antibodies or antigen binding fragments thereof derived from one species of mammals (e.g., mouse, rat, rabbit, etc.) with the desired specificity, affinity and capability, while the constant regions are homologous to the sequences in antibodies or antigen binding fragments thereof derived from another species of mammals (usually a human) to avoid eliciting an immune response in that species.

The term “humanized antibody.” or antigen binding fragment thereof, refers to antibodies or antigen binding fragments that contain minimal non-human (e.g., murine) sequences. Typically, humanized antibodies or antigen binding fragments thereof are human immunoglobulins in which residues from one or more CDRs are replaced with residues from one or more CDRs of a non-human species (e.g., mouse, rat, rabbit, hamster) that have the desired specificity, affinity, and capability (Jones et al., Nature 321:522-525 (1986): Riechmann et al., Nature 332: 323-327 (1988): Verhoeyen et al., Science 239: 1534-1536 (1988)). In general, a humanized antibody or antigen binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. In some aspects, the FR residues of a human immunoglobulin can be also replaced with the corresponding residues in an antibody or fragment from a non-human species that has the desired specificity, affinity, and capability. The humanized antibody or antigen binding fragment thereof can be further modified by the substitution of additional residues either in the FR and/or within the CDRs to refine and optimize the antibody or antigen-binding fragment thereof specificity, affinity, and/or capability. A humanized antibody or antigen binding fragment thereof can also comprise at least a portion of a constant region, typically that of a human immunoglobulin. Examples of methods used to generate humanized antibodies are known and described, for example, in U.S. Pat. No. 5,225,539; Roguska et al., Proc. Natl. Acad. Sci., USA. 91 (3): 969-973 (1994), and Roguska et al., Protein Eng. 9 (10): 895-904 (1996).

The term “human antibody” or antigen binding fragment thereof, as used herein, means an antibody or antigen binding fragment thereof having an amino acid sequence derived from a human immunoglobulin gene locus, where such antibody or antigen binding fragment is made using recombinant techniques known in the art. This definition of a human antibody or antigen binding fragment thereof includes intact or full-length antibodies and fragments thereof.

A polypeptide, polypeptide complex, antibody, antigen binding fragment thereof, polynucleotide, vector or host cell which is “isolated” is a polypeptide, polypeptide complex, antibody, antigen binding fragment thereof, polynucleotide, vector or host cell which is in a form not found in nature. Isolated polypeptides, polypeptide complexes, antibodies, antigen binding fragments thereof, polynucleotides, vectors or host cells include those which have been purified to a degree that they are no longer in a form in which they are found in nature. In some aspects, a polypeptide, polypeptide complex, antibody, antigen binding fragment thereof, polynucleotide, vector or host cell which is isolated is substantially pure. As used herein. “substantially pure” refers to material which is at least 50% pure (i.e., free from contaminants), at least 90% pure, at least 95% pure, at least 98% pure, or at least 99% pure.

The terms “polypeptide.” “peptide.” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, or antigen binding fragments thereof, in some aspects, the polypeptides can occur as single chains or associated chains.

The use of the alternative (e.g., “or”) should be understood to mean either one, both, or a combination thereof of the alternatives. As used herein, the indefinite articles “a” or “an” should be understood to refer to “one or more” of any recited or enumerated component.

As used herein the term “and/or” is to be taken as specific disclosure of each of the two specified features or components with or without the other. Thus, the term “and/or” as used in a phrase such as “A and/or B” herein is intended to include “A and B.” “A or B.” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising.” “having.” or the like, otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example. “about” can mean within 1 or more than 1 standard deviation per the practice in the art. Alternatively. “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As used herein, the terms “polyreactivity” and “poly-reactivity” are used interchangeably and mean the ability of an antigen binding polypeptide, antigen binding polypeptide complex, polypeptide, antibody or antigen binding fragment thereof (e.g., an anti-SARS-CoV-2 virus antibody or antigen binding fragment thereof) disclosed herein to bind to multiple molecularly distinct antigens. Polyreactivity can be measured, for example, using enzyme-linked immunosorbent assay (ELISA) or surface plasmon resonance (SPR), as described herein.

As described herein, any numerical range, concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology. Juo, Pei-Show. 2nd ed., 2002. CRC Press; The Dictionary of Cell and Molecular Biology. 5th ed., 2013. Academic Press; and the Oxford Dictionary Of Biochemistry And Molecular Biology. 2006. Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined herein are more fully defined by reference to the specification in its entirety.

Various aspects are described in further detail in the following sections.

### Antigen Binding Polypeptides and Polypeptide Complexes-1

Provided herein are antigen binding polypeptides and antigen binding polypeptide complexes having certain structural features.

In some aspects, the antigen binding polypeptides provided herein comprise one or more immunoglobulin light chain variable regions (VL) and/or one or more immunoglobulin heavy chain variable regions (VH). In some aspects, the one or more VL (herein also referred to as, e.g., VL1, VL2, VL3, or VL4) specifically binds to a SARS-CoV-2 antigen. In some aspects, the one or more VL specifically binds to a SARS-CoV-2 protein (e.g., a SARS-CoV-2 spike protein). In some aspects, the one or more VH (herein also referred to as, e.g., VH1, VH2, VH3, or VH4) specifically binds to a SARS-CoV-2 antigen. In some aspects, the one or more VH specifically binds to a SARS-CoV-2 protein (e.g., a SARS-CoV-2 spike protein).

In some aspects, the antigen binding polypeptides provided herein comprise two VLs, each of the two VLs comprises a CDR1, a CDR2, and a CDR3 that are the same. In some aspects, each of the two VLs comprises a CDR1, a CDR2, and a CDR3 that are different between the two VLs.

In some aspects, the antigen binding polypeptides provided herein comprise more than two VLs. In some aspects, each of the more than two VLs comprises a CDR1, a CDR2, and a CDR3 that are the same. In some aspects, each of the more than two VLs comprises a CDR1, a CDR2, and a CDR3 that are different. In some aspects, some of the more than two VLs comprise a CDR1, a CDR2, and a CDR3 that are the same, and some of the more than two VLs comprise a CDR1, a CDR2, and a CDR3 that are different.

In some aspects, the antigen binding polypeptides provided herein comprise two VHs, each of the two VHs comprises a CDR1, a CDR2, and a CDR3 that are the same. In some aspects, each of the two VHs comprises a CDR1, a CDR2, and a CDR3 that are different between the two VHs.

In some aspects, the antigen binding polypeptides provided herein comprise more than two VHs. In some aspects, each of the more than two VHs comprises a CDR1, a CDR2, and a CDR3 that are the same. In some aspects, each of the more than two VHs comprises a CDR1, a CDR2, and a CDR3 that are different. In some aspects, some of the more than two VHs comprises a CDR1, a CDR2, and a CDR3 that are the same, and some of the more than two VHs comprises a CDR1, a CDR2, and a CDR3 that are different.

In some aspects, the antigen binding polypeptides provided herein comprise two VLs. In some aspects, the two VLs are the same. In some aspects, the two VLs are different.

In some aspects, the antigen binding polypeptides provided herein comprise more than two VLs. In some aspects, each of the more than two VLs is the same. In some aspects, each of the more than two VLs is different. In some aspects, some of the more than two VLs are the same, and some of the more than two VLs are different.

In some aspects, the antigen binding polypeptides provided herein comprise two VHs. In some aspects, the two VHs are the same. In some aspects, the two VHs are different.

In some aspects, the antigen binding polypeptides provided herein comprise more than two VHs. In some aspects, each of the more than two VHs is the same. In some aspects, each of the more than two VHs is different. In some aspects, some of the more than two VHs are the same, and some of the more than two VHs are different.

In some aspects, the antigen binding polypeptides provided herein comprise an immunoglobulin heavy chain constant region 1 (CH1). In some aspects, the antigen binding polypeptides provided herein comprise an immunoglobulin light chain constant region or domain (CL).

In some aspects, the antigen binding polypeptides provided herein comprise one or more optional amino acid linkers.

In some aspects, the antigen binding polypeptides provided herein have a structure, from amino-terminus to carboxy-terminus, represented by:
VL1-L1-CL-L2-VH1-L3-CH1;
VL1-L1-CH1-L2-VH1-L3-CL;
VH1-L1-CL-L2-VL1-L3-CH1; or
VH1-L1-CH1-L2-VL1-L3-CL;
wherein:
VL1 is an immunoglobulin light chain variable region that specifically binds to a severe acute respiratory syndrome coronavirus 2 (SARS-CoV-2) protein;
VH1 is an immunoglobulin heavy chain variable region that specifically binds to a SARS-CoV-2 protein;
CL is an immunoglobulin light chain constant region;
CH1 is an immunoglobulin heavy chain constant region 1; and
L1-L3 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, and 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (v) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (vi) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising a CDR1 having an amino acid sequence that is at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence that is at least 65%, at least 70%, at least 75%, at least 80%, at least 85%, at least 90%, at least 95%, at least 99%, or 100% identical to amino acid sequence GAS, and a CDR3 having an amino acid sequence that is at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VH1 comprising a CDR1 having an amino acid sequence that is at least 65%, at least 70%, at least 75%, at least 80%, at least 85%, at least 90%, at least 95%, at least 99%, or 100% identical to SEQ ID NO: 12 or 1057, a CDR2 having an amino acid sequence that is at least 65%, at least 70%, at least 75%, at least 80%, at least 85%, at least 90%, at least 95%, at least 99%, or 100% identical to SEQ ID NO: 6 or 1058, and a CDR3 having an amino acid sequence that is at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise (i) a VL1 comprising a CDR1 having an amino acid sequence that is at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence that is at least 65%, at least 70%, at least 75%, at least 80%, at least 85%, at least 90%, at least 95%, at least 99%, or 100% identical to amino acid sequence GAS, and a CDR3 having an amino acid sequence that is at least 65%, at least 70%, at least 75%, at least 80%, at least 85%, at least 90%, at least 95%, at least 99%, or 100% identical to SEQ ID NO: 10; and (ii) a VH1 comprising a CDR1 having an amino acid sequence that is at least 65%, at least 70%, at least 75%, at least 80%, at least 85%, at least 90%, at least 95%, at least 99%, or 100% identical to SEQ ID NO: 12 or 1057, a CDR2 having an amino acid sequence that is at least 65%, at least 70%, at least 75%, at least 80%, at least 85%, at least 90%, at least 95%, at least 99%, or 100% identical to SEQ ID NO: 6 or 1058, and a CDR3 having an amino acid sequence that is at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 11, and (ii) a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147, and (ii) a VH1 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides disclosed herein, comprise one or more CL region, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides disclosed herein, comprise one or more CH1 region, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional amino acid linkers. The one or more optional amino acid linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VL1-L1-CL-L2-VL2-L3-VH2-L4-VH1-L5-CH1;
VL1-L1-CL-L2-VH2-L3-VL2-L4-VH1-L5-CH1;
VL1-L1-CH1-L2-VL2-L3-VH2-L4-VH1-L5-CL;
VL1-L1-CH1-L2-VH2-L3-VL2-L4-VH1-L5-CL;
VH1-L1-CL-L2-VL2-L3-VH2-L4-VL1-L5-CH1;
VH1-L1-CL-L2-VH2-L3-VL2-L4-VL1-L5-CH1;
VH1-L1-CH1-L2-VL2-L3-VH2-L4-VL1-L5-CL;
VH1-L1-CH1-L2-VH2-L3-VL2-L4-VL1-L5-CL;
VL2-L1-CL-L2-VL1-L3-VH1-L4-VH2-L5-CH1;
VL2-L1-CL-L2-VH1-L3-VL1-L4-VH2-L5-CH1;
VL2-L1-CH1-L2-VL1-L3-VH1-L4-VH2-L5-CL;
VL2-L1-CH1-L2-VH1-L3-VL1-L4-VH2-L5-CL;
VH2-L1-CL-L2-VL1-L3-VH1-L4-VL2-L5-CH1;
VH2-L1-CL-L2-VH1-L3-VL1-L4-VL2-L5-CH1;
VH2-L1-CH1-L2-VL1-L3-VH1-L4-VL2-L5-CL; or
VH2-L1-CH1-L2-VH1-L3-VL1-L4-VL2-L5-CL;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
CL is an immunoglobulin light chain constant region;
CH1 is an immunoglobulin heavy chain constant region 1; and
L1-L5 are optional amino acid linkers.

In some aspects, the antigen binding polypeptides provided herein have a structure, from amino-terminus to carboxy-terminus, represented by:
VL1-CL-L1-VL2-L2-VH2-L3-VH1-CH1;
VL1-CL-L1-VH2-L2-VL2-L3-VH1-CH1;
VL1-CH1-L1-VL2-L2-VH2-L3-VH1-CL;
VL1-CH1-L1-VH2-L2-VL2-L3-VH1-CL;
VH1-CL-L1-VL2-L2-VH2-L3-VL1-CH1;
VH1-CL-L1-VH2-L2-VL2-L3-VL1-CH1;
VH1-CH1-L1-VL2-L2-VH2-L3-VL1-CL;
VH1-CH1-L1-VH2-L2-VL2-L3-VL1-CL;
VL2-CL-L1-VL1-L2-VH1-L3-VH2-CH1;
VL2-CL-L1-VH1-L2-VL1-L3-VH2-CH1;
VL2-CH1-L1-VL1-L2-VH1-L3-VH2-CL;
VL2-CH1-L1-VH1-L2-VL1-L3-VH2-CL;
VH2-CL-L1-VL1-L2-VH1-L3-VL2-CH1;
VH2-CL-L1-VH1-L2-VL1-L3-VL2-CH1;
VH2-CH1-L1-VL1-L2-VH1-L3-VL2-CL; or
VH2-CH1-L1-VH1-L2-VL1-L3-VL2-CL;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
CL is an immunoglobulin light chain constant region;
CH1 is an immunoglobulin heavy chain constant region 1; and
L1-L3 are amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875.882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 25.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 17; (ii) a VL2 comprising an amino acid sequence that is at least 65%, at least 70%, at least 75%, at least 80%, at least 85%, at least 90%, at least 95%, at least 99%, or 100% identical to SEQ ID NO: 25; (iii) a VH1 comprising an amino acid sequence that is at least 65%, at least 70%, at least 75%, at least 80%, at least 85%, at least 90%, at least 95%, at least 99%, or 100% identical to SEQ ID NO: 21 or 22; and (iv) a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 17; (ii) a VL2 comprising an amino acid sequence that is at least 65%, at least 70%, at least 75%, at least 80%, at least 85%, at least 90%, at least 95%, at least 99%, or 100% identical to SEQ ID NO: 11; (iii) a VH1 comprising an amino acid sequence that is at least 65%, at least 70%, at least 75%, at least 80%, at least 85%, at least 90%, at least 95%, at least 99%, or 100% identical to SEQ ID NO: 21 or 22; and (iv) a VH2 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 25.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 25; (ii) a VL2 comprising an amino acid sequence that is at least 65%, at least 70%, at least 75%, at least 80%, at least 85%, at least 90%, at least 95%, at least 99%, or 100% identical to SEQ ID NO: 11; (iii) a VH1 comprising an amino acid sequence that is at least 65%, at least 70%, at least 75%, at least 80%, at least 85%, at least 90%, at least 95%, at least 99%, or 100% identical to SEQ ID NO: 29; and (iv) a VH2 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 25.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 4.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 8.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 25; (ii) a VL2 comprising an amino acid sequence that is at least 65%, at least 70%, at least 75%, at least 80%, at least 85%, at least 90%, at least 95%, at least 99%, or 100% identical to SEQ ID NO: 4; (iii) a VH1 comprising an amino acid sequence that is at least 65%, at least 70%, at least 75%, at least 80%, at least 85%, at least 90%, at least 95%, at least 99%, or 100% identical to SEQ ID NO: 29; and (iv) a VH2 comprising an amino acid sequence that is at least 65%, at least 70%, at least 75%, at least 80%, at least 85%, at least 90%, at least 95%, at least 99%, or 100% identical to SEQ ID NO: 8.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 33 or 39.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 37.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 17; (ii) a VL2 comprising an amino acid sequence that is at least 65%, at least 70%, at least 75%, at least 80%, at least 85%, at least 90%, at least 95%, at least 99%, or 100% identical to SEQ ID NO: 33 or 39; (iii) a VH1 comprising an amino acid sequence that is at least 65%, at least 70%, at least 75%, at least 80%, at least 85%, at least 90%, at least 95%, at least 99%, or 100% identical to SEQ ID NO: 21 or 22; and (iv) a VH2 comprising an amino acid sequence that is at least 65%, at least 70%, at least 75%, at least 80%, at least 85%, at least 90%, at least 95%, at least 99%, or 100% identical to SEQ ID NO: 37.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 67.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 11; (ii) a VL2 comprising an amino acid sequence that is at least 65%, at least 70%, at least 75%, at least 80%, at least 85%, at least 90%, at least 95%, at least 99%, or 100% identical to SEQ ID NO: 67; (iii) a VH1 comprising an amino acid sequence that is at least 65%, at least 70%, at least 75%, at least 80%, at least 85%, at least 90%, at least 95%, at least 99%, or 100% identical to SEQ ID NO: 14; and (iv) a VH2 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 154.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 11; (ii) a VL2 comprising an amino acid sequence that is at least 65%, at least 70%, at least 75%, at least 80%, at least 85%, at least 90%, at least 95%, at least 99%, or 100% identical to SEQ ID NO: 154; (iii) a VH1 comprising an amino acid sequence that is at least 65%, at least 70%, at least 75%, at least 80%, at least 85%, at least 90%, at least 95%, at least 99%, or 100% identical to SEQ ID NO: 14; and (iv) a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VL2 comprising an amino acid sequence that is at least 65%, at least 70%, at least 75%, at least 80%, at least 85%, at least 90%, at least 95%, at least 99%, or 100% identical to SEQ ID NO: 11; (iii) a VH1 comprising an amino acid sequence that is at least 65%, at least 70%, at least 75%, at least 80%, at least 85%, at least 90%, at least 95%, at least 99%, or 100% identical to SEQ ID NO: 151; and (iv) a VH2 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 67.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 67; (ii) a VL2 comprising an amino acid sequence that is at least 65%, at least 70%, at least 75%, at least 80%, at least 85%, at least 90%, at least 95%, at least 99%, or 100% identical to SEQ ID NO: 17; (iii) a VH1 comprising an amino acid sequence that is at least 65%, at least 70%, at least 75%, at least 80%, at least 85%, at least 90%, at least 95%, at least 99%, or 100% identical to SEQ ID NO: 71 or 626; and (iv) a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 67.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 154.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 67; (ii) a VL2 comprising an amino acid sequence that is at least 65%, at least 70%, at least 75%, at least 80%, at least 85%, at least 90%, at least 95%, at least 99%, or 100% identical to SEQ ID NO: 154; (iii) a VH1 comprising an amino acid sequence that is at least 65%, at least 70%, at least 75%, at least 80%, at least 85%, at least 90%, at least 95%, at least 99%, or 100% identical to SEQ ID NO: 71 or 626; and (iv) a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 67.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 17; (ii) a VL2 comprising an amino acid sequence that is at least 65%, at least 70%, at least 75%, at least 80%, at least 85%, at least 90%, at least 95%, at least 99%, or 100% identical to SEQ ID NO: 67; (iii) a VH1 comprising an amino acid sequence that is at least 65%, at least 70%, at least 75%, at least 80%, at least 85%, at least 90%, at least 95%, at least 99%, or 100% identical to SEQ ID NO: 21 or 22; and (iv) a VH2 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 154.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 67.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 154; (ii) a VL2 comprising an amino acid sequence that is at least 65%, at least 70%, at least 75%, at least 80%, at least 85%, at least 90%, at least 95%, at least 99%, or 100% identical to SEQ ID NO: 67; (iii) a VH1 comprising an amino acid sequence that is at least 65%, at least 70%, at least 75%, at least 80%, at least 85%, at least 90%, at least 95%, at least 99%, or 100% identical to SEQ ID NO: 158; and (iv) a VH2 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 17; (ii) a VL2 comprising an amino acid sequence that is at least 65%, at least 70%, at least 75%, at least 80%, at least 85%, at least 90%, at least 95%, at least 99%, or 100% identical to SEQ ID NO: 147; (iii) a VH1 comprising an amino acid sequence that is at least 65%, at least 70%, at least 75%, at least 80%, at least 85%, at least 90%, at least 95%, at least 99%, or 100% identical to SEQ ID NO: 21 or 22; and (iv) a VH2 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 67.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 67; (ii) a VL2 comprising an amino acid sequence that is at least 65%, at least 70%, at least 75%, at least 80%, at least 85%, at least 90%, at least 95%, at least 99%, or 100% identical to SEQ ID NO: 147; (iii) a VH1 comprising an amino acid sequence that is at least 65%, at least 70%, at least 75%, at least 80%, at least 85%, at least 90%, at least 95%, at least 99%, or 100% identical to SEQ ID NO: 71 or 626; and (iv) a VH2 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 154.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VL2 comprising an amino acid sequence that is at least 65%, at least 70%, at least 75%, at least 80%, at least 85%, at least 90%, at least 95%, at least 99%, or 100% identical to SEQ ID NO: 154; (iii) a VH1 comprising an amino acid sequence that is at least 65%, at least 70%, at least 75%, at least 80%, at least 85%, at least 90%, at least 95%, at least 99%, or 100% identical to SEQ ID NO: 151; and (iv) a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 174.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 178.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 174; (ii) a VL2 comprising an amino acid sequence that is at least 65%, at least 70%, at least 75%, at least 80%, at least 85%, at least 90%, at least 95%, at least 99%, or 100% identical to SEQ ID NO: 147; (iii) a VH1 comprising an amino acid sequence that is at least 65%, at least 70%, at least 75%, at least 80%, at least 85%, at least 90%, at least 95%, at least 99%, or 100% identical to SEQ ID NO: 178; and (iv) a VH2 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 140.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 154.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 144.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 140; (ii) a VL2 comprising an amino acid sequence that is at least 65%, at least 70%, at least 75%, at least 80%, at least 85%, at least 90%, at least 95%, at least 99%, or 100% identical to SEQ ID NO: 154; (iii) a VH1 comprising an amino acid sequence that is at least 65%, at least 70%, at least 75%, at least 80%, at least 85%, at least 90%, at least 95%, at least 99%, or 100% identical to SEQ ID NO: 144; and (iv) a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VL2 comprising an amino acid sequence that is at least 65%, at least 70%, at least 75%, at least 80%, at least 85%, at least 90%, at least 95%, at least 99%, or 100% identical to SEQ ID NO: 147; (iii) a VH1 comprising an amino acid sequence that is at least 65%, at least 70%, at least 75%, at least 80%, at least 85%, at least 90%, at least 95%, at least 99%, or 100% identical to SEQ ID NO: 151; and (iv) a VH2 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 154.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 154.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 154; (ii) a VL2 comprising an amino acid sequence that is at least 65%, at least 70%, at least 75%, at least 80%, at least 85%, at least 90%, at least 95%, at least 99%, or 100% identical to SEQ ID NO: 154; (iii) a VH1 comprising an amino acid sequence that is at least 65%, at least 70%, at least 75%, at least 80%, at least 85%, at least 90%, at least 95%, at least 99%, or 100% identical to SEQ ID NO: 158; and (iv) a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 11; (ii) a VL2 comprising an amino acid sequence that is at least 65%, at least 70%, at least 75%, at least 80%, at least 85%, at least 90%, at least 95%, at least 99%, or 100% identical to SEQ ID NO: 11; (iii) a VH1 comprising an amino acid sequence that is at least 65%, at least 70%, at least 75%, at least 80%, at least 85%, at least 90%, at least 95%, at least 99%, or 100% identical to SEQ ID NO: 14; and (iv) a VH2 comprising an amino acid sequence that is at least 65%, at least 70%, at least 75%, at least 80%, at least 85%, at least 90%, at least 95%, at least 99%, or 100% identical to SEQ ID NO: 14.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprised in the antigen binding polypeptides provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147. In some aspects, at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (ix) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (x) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (x) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 9 or 1055; (viii) a CDR2 having an amino acid sequence that is at least 65%, at least 70%, at least 75%, at least 80%, at least 85%, at least 90%, at least 95%, at least 99%, or 100% identical to SEQ ID NO: 1056 or an amino acid sequence that is at least 65%, at least 70%, at least 75%, at least 80%, at least 85%, at least 90%, at least 95%, at least 99%, or 100% identical to amino acid sequence GAS; and (ix) a CDR3 having an amino acid sequence that is at least 65%, at least 70%, at least 75%, at least 80%, at least 85%, at least 90%, at least 95%, at least 99%, or 100% identical to SEQ ID NO: 10; and a VH2 comprising (x) a CDR1 having an amino acid sequence that is at least 65%, at least 70%, at least 75%, at least 80%, at least 85%, at least 90%, at least 95%, at least 99%, or 100% identical to SEQ ID NO: 12 or 1057; (xi) a CDR2 having an amino acid sequence that is at least 65%, at least 70%, at least 75%, at least 80%, at least 85%, at least 90%, at least 95%, at least 99%, or 100% identical to SEQ ID NO: 6 or 1058; and (xii) a CDR3 having an amino acid sequence that is at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to SEQ ID NO: 11; and (iv) a VH2 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 152 or 1045; (viii) a CDR2 having an amino acid sequence that is at least 65%, at least 70%, at least 75%, at least 80%, at least 85%, at least 90%, at least 95%, at least 99%, or 100% identical to SEQ ID NO: 1046 or an amino acid sequence that is at least 65%, at least 70%, at least 75%, at least 80%, at least 85%, at least 90%, at least 95%, at least 99%, or 100% identical to amino acid sequence GAS; and (ix) a CDR3 having an amino acid sequence that is at least 65%, at least 70%, at least 75%, at least 80%, at least 85%, at least 90%, at least 95%, at least 99%, or 100% identical to SEQ ID NO: 153 or 766; and a VH2 comprising (x) a CDR1 having an amino acid sequence that is at least 65%, at least 70%, at least 75%, at least 80%, at least 85%, at least 90%, at least 95%, at least 99%, or 100% identical to SEQ ID NO: 155 or 1047; (xi) a CDR2 having an amino acid sequence that is at least 65%, at least 70%, at least 75%, at least 80%, at least 85%, at least 90%, at least 95%, at least 99%, or 100% identical to SEQ ID NO: 156 or 1048; and (xii) a CDR3 having an amino acid sequence that is at least 65%, at least 70%, at least 75%, at least 80%, at least 85%, at least 90%, at least 95%, at least 99%, or 100% identical to SEQ ID NO: 157, 767, or 1049.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to SEQ ID NO: 154; and (iv) a VH2 comprising an amino acid sequence that is at least 65%, at least 70%, at least 75%, at least 80%, at least 85%, at least 90%, at least 95%, at least 99%, or 100% identical to SEQ ID NO: 158.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 65 or 627; (viii) a CDR2 having an amino acid sequence that is at least 65%, at least 70%, at least 75%, at least 80%, at least 85%, at least 90%, at least 95%, at least 99%, or 100% identical to SEQ ID NO: 628 or an amino acid sequence that is at least 65%, at least 70%, at least 75%, at least 80%, at least 85%, at least 90%, at least 95%, at least 99%, or 100% identical to amino acid sequence SDS; and (ix) a CDR3 having an amino acid sequence that is at least 65%, at least 70%, at least 75%, at least 80%, at least 85%, at least 90%, at least 95%, at least 99%, or 100% identical to SEQ ID NO: 66; and a VH2 comprising (x) a CDR1 having an amino acid sequence that is at least 65%, at least 70%, at least 75%, at least 80%, at least 85%, at least 90%, at least 95%, at least 99%, or 100% identical to SEQ ID NO: 68 or 629; (xi) a CDR2 having an amino acid sequence that is at least 65%, at least 70%, at least 75%, at least 80%, at least 85%, at least 90%, at least 95%, at least 99%, or 100% identical to SEQ ID NO: 69 or 630; and (xii) a CDR3 having an amino acid sequence that is at least 65%, at least 70%, at least 75%, at least 80%, at least 85%, at least 90%, at least 95%, at least 99%, or 100% identical to SEQ ID NO: 70 or 631.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to SEQ ID NO: 67; and (iv) a VH2 comprising an amino acid sequence that is at least 65%, at least 70%, at least 75%, at least 80%, at least 85%, at least 90%, at least 95%, at least 99%, or 100% identical to SEQ ID NO: 71 or 626.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 138 or 1065; (viii) a CDR2 having an amino acid sequence that is at least 65%, at least 70%, at least 75%, at least 80%, at least 85%, at least 90%, at least 95%, at least 99%, or 100% identical to SEQ ID NO: 1061 or an amino acid sequence that is at least 65%, at least 70%, at least 75%, at least 80%, at least 85%, at least 90%, at least 95%, at least 99%, or 100% identical to amino acid sequence DAS; and (ix) a CDR3 having an amino acid sequence that is at least 65%, at least 70%, at least 75%, at least 80%, at least 85%, at least 90%, at least 95%, at least 99%, or 100% identical to SEQ ID NO: 139, 856, or 1066; and a VH2 comprising (x) a CDR1 having an amino acid sequence that is at least 65%, at least 70%, at least 75%, at least 80%, at least 85%, at least 90%, at least 95%, at least 99%, or 100% identical to SEQ ID NO: 141 or 1067; (xi) a CDR2 having an amino acid sequence that is at least 65%, at least 70%, at least 75%, at least 80%, at least 85%, at least 90%, at least 95%, at least 99%, or 100% identical to SEQ ID NO: 142 or 1068; and (xii) a CDR3 having an amino acid sequence that is at least 65%, at least 70%, at least 75%, at least 80%, at least 85%, at least 90%, at least 95%, at least 99%, or 100% identical to SEQ ID NO: 143, 857, or 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to SEQ ID NO: 140; and (iv) a VH2 comprising an amino acid sequence that is at least 65%, at least 70%, at least 75%, at least 80%, at least 85%, at least 90%, at least 95%, at least 99%, or 100% identical to SEQ ID NO: 144.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 172 or 1060; (viii) a CDR2 having an amino acid sequence that is at least 65%, at least 70%, at least 75%, at least 80%, at least 85%, at least 90%, at least 95%, at least 99%, or 100% identical to SEQ ID NO: 1061 or an amino acid sequence that is at least 65%, at least 70%, at least 75%, at least 80%, at least 85%, at least 90%, at least 95%, at least 99%, or 100% identical to amino acid sequence DAS; and (ix) a CDR3 having an amino acid sequence that is at least 65%, at least 70%, at least 75%, at least 80%, at least 85%, at least 90%, at least 95%, at least 99%, or 100% identical to SEQ ID NO: 173 or 792; and a VH2 comprising (x) a CDR1 having an amino acid sequence that is at least 65%, at least 70%, at least 75%, at least 80%, at least 85%, at least 90%, at least 95%, at least 99%, or 100% identical to SEQ ID NO: 175 or 1062; (xi) a CDR2 having an amino acid sequence that is at least 65%, at least 70%, at least 75%, at least 80%, at least 85%, at least 90%, at least 95%, at least 99%, or 100% identical to SEQ ID NO: 176 or 1063; and (xii) a CDR3 having an amino acid sequence that is at least 65%, at least 70%, at least 75%, at least 80%, at least 85%, at least 90%, at least 95%, at least 99%, or 100% identical to SEQ ID NO: 177, 793, or 1064.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to SEQ ID NO: 174; and (iv) a VH2 comprising an amino acid sequence that is at least 65%, at least 70%, at least 75%, at least 80%, at least 85%, at least 90%, at least 95%, at least 99%, or 100% identical to SEQ ID NO: 178.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (viii) a CDR2 having an amino acid sequence that is at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (ix) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and a VH2 comprising (x) a CDR1 having an amino acid sequence that is at least 65%, at least 70%, at least 75%, at least 80%, at least 85%, at least 90%, at least 95%, at least 99%, or 100% identical to SEQ ID NO: 148 or 1052; (xi) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (xii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to SEQ ID NO: 147; and (iv) a VH2 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VL1-L1-VH1-L2-VL2-L3-CL-L4-VH2-L5-CH1;
VL1-L1-VH1-L2-VL2-L3-CH1-L4-VH2-L5-CL;
VL1-L1-VH1-L2-VH2-L3-CL-L4-VL2-L5-CH1;
VL1-L1-VH1-L2-VH2-L3-CH1-L4-VL2-L5-CL;
VL1-L1-VL2-L2-VH1-L3-CL-L4-VH2-L5-CH1;
VL1-L1-VL2-L2-VH1-L3-CH1-L4-VH2-L5-CL;
VL1-L1-VH2-L2-VH1-L3-CL-L4-VL2-L5-CH1;
VL1-L1-VH2-L2-VH1-L3-CH1-L4-VL2-L5-CL;
VH1-L1-VL1-L2-VL2-L3-CL-L4-VH2-L5-CH1;
VH1-L1-VL1-L2-VL2-L3-CH1-L4-VH2-L5-CL;
VH1-L1-VL1-L2-VH2-L3-CL-L4-VL2-L5-CH1;
VH1-L1-VL1-L2-VH2-L3-CH1-L4-VL2-L5-CL;
VH1-L1-VL2-L2-VL1-L3-CL-L4-VH2-L5-CH1;
VH1-L1-VL2-L2-VL1-L3-CH1-L4-VH2-L5-CL;
VH1-L1-VH2-L2-VL1-L3-CL-L4-VL2-L5-CH1;
VH1-L1-VH2-L2-VL1-L3-CH1-L4-VL2-L5-CL;
VL2-L1-VL1-L2-VH2-L3-CL-L4-VH1-L5-CH1;
VL2-L1-VL1-L2-VH2-L3-CH1-L4-VH1-L5-CL;
VL2-L1-VH1-L2-VH2-L3-CL-L4-VL1-L5-CH1;
VL2-L1-VH1-L2-VH2-L3-CH1-L4-VL1-L5-CL;
VL2-L1-VH2-L2-VL1-L3-CL-L4-VH1-L5-CH1;
VL2-L1-VH2-L2-VL1-L3-CH1-L4-VH1-L5-CL;
VL2-L1-VH2-L2-VH1-L3-CL-L4-VL1-L5-CH1;
VL2-L1-VH2-L2-VH1-L3-CH1-L4-VL1-L5-CL;
VH2-L1-VL1-L2-VL2-L3-CL-L4-VH1-L5-CH1;
VH2-L1-VL1-L2-VL2-L3-CH1-L4-VH1-L5-CL;
VH2-L1-VH1-L2-VL2-L3-CL-L4-VL1-L5-CH1;
VH2-L1-VH1-L2-VL2-L3-CH1-L4-VL1-L5-CL;
VH2-L1-VL2-L2-VL1-L3-CL-L4-VH1-L5-CH1;
VH2-L1-VL2-L2-VL1-L3-CH1-L4-VH1-L5-CL;
VH2-L1-VL2-L2-VH1-L3-CL-L4-VL1-L5-CH1; or
VH2-L1-VL2-L2-VH1-L3-CH1-L4-VL1-L5-CL;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
CL is an immunoglobulin light chain constant region;
CH1 is an immunoglobulin heavy chain constant region 1; and
L1-L5 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 25.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 25; (ii) a VL2 comprising an amino acid sequence that is at least 65%, at least 70%, at least 75%, at least 80%, at least 85%, at least 90%, at least 95%, at least 99%, or 100% identical to SEQ ID NO: 17; (iii) a VH1 comprising an amino acid sequence that is at least 65%, at least 70%, at least 75%, at least 80%, at least 85%, at least 90%, at least 95%, at least 99%, or 100% identical to SEQ ID NO: 29; and (iv) a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 11; (ii) a VL2 comprising an amino acid sequence that is at least 65%, at least 70%, at least 75%, at least 80%, at least 85%, at least 90%, at least 95%, at least 99%, or 100% identical to SEQ ID NO: 17; (iii) a VH1 comprising an amino acid sequence that is at least 65%, at least 70%, at least 75%, at least 80%, at least 85%, at least 90%, at least 95%, at least 99%, or 100% identical to SEQ ID NO: 14; and (iv) a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 25.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical SEQ ID NO: 14.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 11; (ii) a VL2 comprising an amino acid sequence that is at least 65%, at least 70%, at least 75%, at least 80%, at least 85%, at least 90%, at least 95%, at least 99%, or 100% identical to SEQ ID NO: 25; (iii) a VH1 comprising an amino acid sequence that is at least 65%, at least 70%, at least 75%, at least 80%, at least 85%, at least 90%, at least 95%, at least 99%, or 100% identical SEQ ID NO: 14; and (iv) a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 4.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 25.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 8.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 4; (ii) a VL2 comprising an amino acid sequence that is at least 65%, at least 70%, at least 75%, at least 80%, at least 85%, at least 90%, at least 95%, at least 99%, or 100% identical to SEQ ID NO: 25; (iii) a VH1 comprising an amino acid sequence that is at least 65%, at least 70%, at least 75%, at least 80%, at least 85%, at least 90%, at least 95%, at least 99%, or 100% identical to SEQ ID NO: 8; and (iv) a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VH1 comprising an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (iii) a VH1 comprising an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 33 or 39.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 37.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprised in the antigen binding polypeptides provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147. In some aspects, at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S. L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by:
VL1-L1-CL-L2-VH1-L3-CH1-L4-VL2-L5-VH2;
VL1-L1-CL-L2-VH1-L3-CH1-L4-VH2-L5-VL2;
VL1-L1-CL-L2-VL2-L3-CH1-L4-VH1-L5-VH2;
VL1-L1-CL-L2-VH2-L3-CH1-L4-VH1-L5-VL2;
VL1-L1-CH1-L2-VH1-L3-CL-L4-VL2-L5-VH2;
VL1-L1-CH1-L2-VH1-L3-CL-L4-VH2-L5-VL2;
VL1-L1-CH1-L2-VL2-L3-CL-L4-VH1-L5-VH2;
VL1-L1-CH1-L2-VH2-L3-CL-L4-VH1-L5-VL2;
VH1-L1-CL-L2-VL1-L3-CH1-L4-VL2-L5-VH2;
VH1-L1-CL-L2-VL1-L3-CH1-L4-VH2-L5-VL2;
VH1-L1-CL-L2-VL2-L3-CH1-L4-VL1-L5-VH2;
VH1-L1-CL-L2-VH2-L3-CH1-L4-VL1-L5-VL2;
VH1-L1-CH1-L2-VL1-L3-CL-L4-VL2-L5-VH2;
VH1-L1-CH1-L2-VL1-L3-CL-L4-VH2-L5-VL2;
VH1-L1-CH1-L2-VL2-L3-CL-L4-VL1-L5-VH2;
VH1-L1-CH1-L2-VH2-L3-CL-L4-VL1-L5-VL2;
VL2-L1-CL-L2-VL1-L3-CH1-L4-VH2-L5-VH1;
VL2-L1-CL-L2-VH1-L3-CH1-L4-VH2-L5-VL1;
VL2-L1-CL-L2-VH2-L3-CH1-L4-VL1-L5-VH1;
VL2-L1-CL-L2-VH2-L3-CH1-L4-VH1-L5-VL1;
VL2-L1-CH1-L2-VL1-L3-CL-L4-VH2-L5-VH1;
VL2-L1-CH1-L2-VH1-L3-CL-L4-VH2-L5-VL1;
VL2-L1-CH1-L2-VH2-L3-CL-L4-VL1-L5-VH1;
VL2-L1-CH1-L2-VH2-L3-CL-L4-VH1-L5-VL1;
VH2-L1-CL-L2-VL1-L3-CH1-L4-VL2-L5-VH1;
VH2-L1-CL-L2-VH1-L3-CH1-L4-VL2-L5-VL1;
VH2-L1-CL-L2-VL2-L3-CH1-L4-VL1-L5-VH1;
VH2-L1-CL-L2-VL2-L3-CH1-L4-VH1-L5-VL1;
VH2-L1-CH1-L2-VL1-L3-CL-L4-VL2-L5-VH1;
VH2-L1-CH1-L2-VH1-L3-CL-L4-VL2-L5-VL1;
VH2-L1-CH1-L2-VL2-L3-CL-L4-VL1-L5-VH1; or
VH2-L1-CH1-L2-VL2-L3-CL-L4-VH1-L5-VL1;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
CL is an immunoglobulin light chain constant region;
CH1 is an immunoglobulin heavy chain constant region 1; and
L1-L5 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a (i) VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 11; (ii) a VL2 comprising an amino acid sequence that is at least 65%, at least 70%, at least 75%, at least 80%, at least 85%, at least 90%, at least 95%, at least 99%, or 100% identical to SEQ ID NO: 17; (iii) a VH1 comprising an amino acid sequence that is at least 65%, at least 70%, at least 75%, at least 80%, at least 85%, at least 90%, at least 95%, at least 99%, or 100% identical to SEQ ID NO: 14; and (iv) a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 25.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 11; (ii) a VL2 comprising an amino acid sequence that is at least 65%, at least 70%, at least 75%, at least 80%, at least 85%, at least 90%, at least 95%, at least 99%, or 100% identical to SEQ ID NO: 25; (iii) a VH1 comprising an amino acid sequence that is at least 65%, at least 70%, at least 75%, at least 80%, at least 85%, at least 90%, at least 95%, at least 99%, or 100% identical to SEQ ID NO: 14; and (iv) a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 4.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 25.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 8.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 4; (ii) a VL2 comprising an amino acid sequence that is at least 65%, at least 70%, at least 75%, at least 80%, at least 85%, at least 90%, at least 95%, at least 99%, or 100% identical to SEQ ID NO: 25; (iii) a VH1 comprising an amino acid sequence that is at least 65%, at least 70%, at least 75%, at least 80%, at least 85%, at least 90%, at least 95%, at least 99%, or 100% identical to SEQ ID NO: 8; and (iv) a VH2 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 25.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 29.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 25; (ii) a VL2 comprising an amino acid sequence that is at least 65%, at least 70%, at least 75%, at least 80%, at least 85%, at least 90%, at least 95%, at least 99%, or 100% identical to SEQ ID NO: 17; (iii) a VH1 comprising an amino acid sequence that is at least 65%, at least 70%, at least 75%, at least 80%, at least 85%, at least 90%, at least 95%, at least 99%, or 100% identical to SEQ ID NO: 29; and (iv) a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326.

In some aspects, the antigen binding polypeptides provided herein comprise a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, the antigen binding polypeptides provided herein comprise a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise (i) a VL1 comprising a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326; (ii) a VL2 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; (iii) a VH1 comprising a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327; and (iv) a VH2 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 33 or 39.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 37.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 33 or 39; (ii) a VL2 comprising an amino acid sequence that is at least 65%, at least 70%, at least 75%, at least 80%, at least 85%, at least 90%, at least 95%, at least 99%, or 100% identical to SEQ ID NO: 17; (iii) a VH1 comprising an amino acid sequence that is at least 65%, at least 70%, at least 75%, at least 80%, at least 85%, at least 90%, at least 95%, at least 99%, or 100% identical to SEQ ID NO: 37; and (iv) a VH2 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprised in the antigen binding polypeptides provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147. In some aspects, at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequences GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by
VL1-L1-VH1-L2-CL; or
VH1-L1-VL1-L2-CL;
wherein:
VL1 is an immunoglobulin light chain variable region that specifically binds to a severe acute respiratory syndrome coronavirus 2 (SARS-CoV-2) protein;
VH1 is an immunoglobulin heavy chain variable region that specifically binds to a SARS-CoV-2 protein;
CL is an immunoglobulin light chain constant region; and
L1-L2 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a (i) VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147, and (ii) a VH1 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequences GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by
VL1-L1-VH1-L2-CH1; or
VH1-L1-VL1-L2-CH1;
wherein:
VL1 is an immunoglobulin light chain variable region that specifically binds to a severe acute respiratory syndrome coronavirus 2 (SARS-CoV-2) protein;
VH1 is an immunoglobulin heavy chain variable region that specifically binds to a SARS-CoV-2 protein;
CH1 is an immunoglobulin heavy chain constant region 1; and
L1-L2 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH1 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16; and a VH1 comprising a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 17.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 17; and a VH1 comprising an amino acid sequence that is at least 65%, at least 70%, at least 75%, at least 80%, at least 85%, at least 90%, at least 95%, at least 99%, or 100% identical to SEQ ID NO: 21 or 22.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10; and a VH1 comprising a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 11; and a VH1 comprising an amino acid sequence that is at least 65%, at least 70%, at least 75%, at least 80%, at least 85%, at least 90%, at least 95%, at least 99%, or 100% identical to SEQ ID NO: 14.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73.

In some aspects, the antigen binding polypeptides provided herein comprise a VH1 comprising a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, the antigen binding polypeptides provided herein comprise a VL1 comprising a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73; and a VH1 comprising a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 74.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 78.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 74; and a VH1 comprising an amino acid sequence that is at least 65%, at least 70%, at least 75%, at least 80%, at least 85%, at least 90%, at least 95%, at least 99%, or 100% identical to SEQ ID NO: 78.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147, and (ii) a VH1 comprising an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by:
VL1-L1-VL2-L2-CH1; or
VL2-L1-VL1-L2-CH1;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
CH1 is an immunoglobulin heavy chain constant region 1; and
L1-L2 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL1 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VL2 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and a VL2 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (v) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (vi) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; and (ii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and a VL2 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (v) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (vi) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; and (ii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptide comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by:
VH1-L1-VH2-L2-CL; or
VH2-L1-VH1-L2-CL;
wherein:
VH1 is a first immunoglobulin heavy chain variable region that specifically binds to a severe acute respiratory syndrome coronavirus 2 (SARS-CoV-2) protein;
VH2 is a second immunoglobulin heavy chain variable region that specifically binds to a SARS-CoV-2 protein
CL is an immunoglobulin light chain constant region; and
L1-L2 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VH1 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH2 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH1 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; and a VH2 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; and a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, at least one of VH1 or VH2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; and a VH2 comprising (iv) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (v) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (vi) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise (i) a VH1 comprising an amino acid sequence that is at least 65%, at least 70%, at least 75%, at least 80%, at least 85%, at least 90%, at least 95%, at least 99%, or 100% identical to SEQ ID NO: 151; and (ii) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; and a VH2 comprising (iv) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (v) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (vi) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise (i) a VH1 comprising an amino acid sequence that is at least 65%, at least 70%, at least 75%, at least 80%, at least 85%, at least 90%, at least 95%, at least 99%, or 100% identical to SEQ ID NO: 151; and (ii) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by:
VL1-L1-VL2-L2-VL3-L3-CH1;
VL1-L1-VL3-L2-VL2-L3-CH1;
VL2-L1-VL1-L2-VL3-L3-CH1;
VL2-L1-VL3-L2-VL1-L3-CH1;
VL3-L1-VL1-L2-VL2-L3-CH1; or
VL3-L1-VL2-L2-VL1-L3-CH1;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VL3 is a third immunoglobulin light chain variable region that specifically binds to a SARS-CoV-2 protein;
CH1 is an immunoglobulin heavy chain constant region 1; and
L1-L3 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT: a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT: a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL3 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT: a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL1 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628.641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT: a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VL2 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT: a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VL3 comprising a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT: a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, at least one of VL1, VL2, or VL3 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VL1, VL2, or VL3 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VL2 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (v) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (vi) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VL3 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iii) a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VL2 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (v) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (vi) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VL3 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iii) a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptide comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by:
VH1-L1-VH2-L2-VH3-L3-CL;
VH1-L1-VH3-L2-VH2-L3-CL;
VH2-L1-VH1-L2-VH3-L3-CL;
VH2-L1-VH3-L2-VH1-L3-CL;
VH3-L1-VH1-L2-VH2-L3-CL; or
VH3-L1-VH2-L2-VH1-L3-CL;
wherein:
VH1 is a first immunoglobulin heavy chain variable region that specifically binds to a severe acute respiratory syndrome coronavirus 2 (SARS-CoV-2) protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
CL is an immunoglobulin light chain constant region; and
L1-L3 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the antigen binding polypeptides provided herein comprise a VH1 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH2 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH3 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH1 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VH2 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; and a VH3 comprising a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; and a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, at least one of VH1, VH2, or VH3 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VH1, VH2, or VH3 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; and a VH2 comprising (iv) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (v) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (vi) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; and a VH3 comprising (vii) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (viii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise (i) a VH1 comprising an amino acid sequence that is at least 65%, at least 70%, at least 75%, at least 80%, at least 85%, at least 90%, at least 95%, at least 99%, or 100% identical to SEQ ID NO: 151; (ii) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884, and (iii) a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; and a VH2 comprising (iv) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (v) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35.69, 76, 149, 156, 630, 142, 176, 1048, 1053, 1058, 1063, and 1068; and (vi) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; and a VH3 comprising (vii) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (viii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (ix) a CDR3 having an amino acid sequence at least 65%, at least 70%, at least 75%, at least 80%, at least 85%, at least 90%, at least 95%, at least 99%, or 100% identical to any one SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise (i) a VH1 comprising an amino acid sequence that is at least 65%, at least 70%, at least 75%, at least 80%, at least 85%, at least 90%, at least 95%, at least 99%, or 100% identical to SEQ ID NO: 151; (ii) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178, and (iii) a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by:
VL1-L1-VL2-L2-VH2-L3-VH1-L4-Fc;
VL1-L1-VH2-L2-VL2-L3-VH1-L4-Fc;
VH1-L1-VH2-L2-VL2-L3-VL1-L4-Fc; or
VH1-L1-VL2-L2-VH2-L3-VL1-L4-Fc;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
L1-L4 are optional amino acid linkers; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34.68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprised in the antigen binding polypeptides provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutations. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprise an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by:
(a)
VL1-L1-VL2-L2-VH2-L3-VH1-L4-CH1-L5-CL;
VL1-L1-VH2-L2-VL2-L3-VH1-L4-CH1-L5-CL;
VH1-L1-VH2-L2-VL2-L3-VL1-L4-CH1-L5-CL;
VH1-L1-VL2-L2-VH2-L3-VL1-L4-CH1-L5-CL;
VL1-L1-VL2-L2-VH2-L3-VH1-L4-CL-L5-CH1;
VL1-L1-VH2-L2-VL2-L3-VH1-L4-CL-L5-CH1;
VH1-L1-VH2-L2-VL2-L3-VL1-L4-CL-L5-CH1; or
VH1-L1-VL2-L2-VH2-L3-VL1-L4-CL-L5-CH1;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
L1-L5 are optional amino acid linkers;
CL is an immunoglobulin light chain constant region; and
CH1 is an immunoglobulin heavy chain constant region 1;
or
(b)
VL1-L1-VL2-L2-VH2-L3-VH1-L4-CH1-L5-CL-L6-Fc;
VL1-L1-VH2-L2-VL2-L3-VH1-L4-CH1-L5-CL-L6-Fc;
VH1-L1-VH2-L2-VL2-L3-VL1-L4-CH1-L5-CL-L6-Fc;
VH1-L1-VL2-L2-VH2-L3-VL1-L4-CH1-L5-CL-L6-Fc;
VL1-L1-VL2-L2-VH2-L3-VH1-L4-CL-L5-CH1-L6-Fc;
VL1-L1-VH2-L2-VL2-L3-VH1-L4-CL-L5-CH1-L6-Fc;
VH1-L1-VH2-L2-VL2-L3-VL1-L4-CL-L5-CH1-L6-Fc; or
VH1-L1-VL2-L2-VH2-L3-VL1-L4-CL-L5-CH1-L6-Fc;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
L1-L6 are optional amino acid linkers;
CL is an immunoglobulin light chain constant region;
CH1 is an immunoglobulin heavy chain constant region 1; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprised in the antigen binding polypeptides provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT.

DAS, DVT, RNS, DVS, EDS, DDS, KDS, VAS, WAS, YTS, EVS, NNN, SYN, and DAT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by:
VL1-L1-VL2-L2-VH2-L3-VH1-L4-Fc-L5-Fc;
VL1-L1-VH2-L2-VL2-L3-VH1-L4-Fc-L5-Fc;
VH1-L1-VH2-L2-VL2-L3-VL1-L4-Fc-L5-Fc; or
VH1-L1-VL2-L2-VH2-L3-VL1-L4-Fc-L5-Fc;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
L1-L5 are optional amino acid linkers; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprised in the antigen binding polypeptides provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147. In some aspects, at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of ID NOs: 6, 19, 27, 35, 69, 76, 149, 156, 630, 142, 176, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptides provided herein have a structure, from amino-terminus to carboxy-terminus, represented by:
VL1-L1-VL2-L2-VH2-L3-VH1;
VL1-L1-VH2-L2-VL2-L3-VH1;
VH1-L1-VH2-L2-VL2-L3-VL1; or
VH1-L1-VL2-L2-VH2-L3-VL1;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein; and
L1-L3 are optional amino acid linkers.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; and a VH2 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprised in the antigen binding polypeptides provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptides provided herein have a structure, from amino-terminus to carboxy-terminus, represented by:
VL1-L1-VH1 or VH1-L1-VL1;
wherein:
VL1 is a first immunoglobulin light chain variable region that specifically binds to a severe acute respiratory syndrome coronavirus 2 (SARS-CoV-2) protein;
VH1 is a first immunoglobulin heavy chain variable region that specifically binds to a SARS-CoV-2 protein; and
L1 is an optional amino acid linker.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34.68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the antigen binding polypeptides provided herein comprise a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; and a VH1 comprising (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the antigen binding polypeptides provided herein comprise (i) a VL1 comprising an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; and (ii) a VH1 comprising an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the VL1 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, the VH1 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the VL1 comprised in the antigen binding polypeptides provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and the VH1 comprised in the antigen binding polypeptides provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the VL1 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, the VH1 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) the VL1 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 147, and (ii) the VH1 comprised in the antigen binding polypeptides provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, an antigen binding polypeptide complex provided herein comprises a first polypeptide, and a second polypeptide; wherein the first polypeptide has a structure represented by VL1-CL-VH1-CH1, VL1-CH1-VH1-CL, VH1-CL-VL1-CH1, or VH1-CH1-VL1-CL; and wherein the second polypeptide has a structure represented by VL2-CL-VL3-VH3-VH2-CH1, VL2-CL-VH3-VL3-VH2-CH1, VL2-CH1-VL3-VH3-VH2-CL, VL2-CH1-VH3-VL3-VH2-CL, VH2-CL-VL3-VH3-VL2-CH1, VH2-CL-VH3-VL3-VL2-CH1, VH2-CH1-VL3-VH3-VL2-CL, VH2-CH1-VH3-VL3-VL2-CL, VL3-CL-VL2-VH2-VH3-CH1, VL3-CL-VH2-VL2-VH3-CH1, VL3-CH1-VL2-VH2-VH3-CL, VL3-CH1-VH2-VL2-VH3-CL, VH3-CL-VL2-VH2-VL3-CH1, VH3-CL-VH2-VL2-VL3-CH1, VH3-CH1-VL2-VH2-VL3-CL, or VH3-CH1-VH2-VL2-VL3-CL.

In some aspects, an antigen binding polypeptide complex provided herein comprises a first polypeptide, and a second polypeptide; wherein the first polypeptide has a structure represented by VL1-L1-CL-L2-VH1-L3-CH1, VL1-L1-CH1-L2-VH1-L3-CL, VH1-L1-CL-L2-VL1-L3-CH1, or VH1-L1- CH1-L2-VL1-L3-CL; and wherein the second polypeptide has a structure represented by VL2-L4-CL-L5-VL3-L6-VH3-L7-VH2-L8-CH1, VL2-L4-CL-L5-VH3-L6-VL3-L7-VH2-L8-CH1, VL2-L4-CH1-L5- VL3-L6-VH3-L7-VH2-L8-CL, VL2-L4-CH1-L5-VH3-L6-VL3-L7-VH2-L8-CL, VH2-L4-CL-L5-VL3-L6-VH3-L7-VL2-L8-CH1, VH2-L4-CL-L5-VH3-L6-VL3-L7-VL2-L8-CH1, VH2-L4-CH1-L5-VL3-L6- VH3-L7-VL2-L8-CL, VH2-L4-CH1-L5-VH3-L6-VL3-L7-VL2-L8-CL, VL3-L4-CL-L5-VL2-L6-VH2-L7-VH3-L8-CH1, VL3-L4-CL-L5-VH2-L6-VL2-L7-VH3-L8-CH1, VL3-L4-CH1-L5-VL2-L6-VH2-L7-VH3-L8-CL, VL3-L4-CH1-L5-VH2-L6-VL2-L7-VH3-L8-CL, VH3-L4-CL-L5-VL2-L6-VH2-L7-VL3-L8-CH1, VH3-L4-CL-L5-VH2-L6-VL2-L7-VL3-L8-CH1, VH3-L4-CH1-L5-VL2-L6-VH2-L7-VL3-L8-CL, or VH3-L4-CH1-L5-VH2-L6-VL2-L7-VL3-L8-CL.

In some aspects, an antigen binding polypeptide complex provided herein comprises a first polypeptide, and a second polypeptide; wherein the first polypeptide has a structure represented by VL1-CL-VH1-CH1-CH2-CH3, VL1-CH1-VH1-CL-CH2-CH3, VH1-CL-VL1-CH1-CH2-CH3, or VH1-CH1-VL1-CL-CH2-CH3; and wherein the second polypeptide has a structure represented by VL2-CL-VL3-VH3-VH2-CH1-CH2-CH3, VL2-CL-VH3-VL3-VH2-CH1-CH2-CH3, VL2-CH1-VL3-VH3-VH2-CL-CH2-CH3, VL2-CH1-VH3-VL3-VH2-CL-CH2-CH3, VH2-CL-VL3-VH3-VL2-CH1-CH2-CH3, VH2-CL-VH3-VL3-VL2-CH1-CH2-CH3, VH2-CH1-VL3-VH3-VL2-CL-CH2-CH3, VH2-CH1-VH3-VL3-VL2-CL-CH2-CH3, VL3-CL-VL2-VH2-VH3-CH1-CH2-CH3, VL3-CL-VH2-VL2-VH3-CH1-CH2-CH3, VL3-CH1-VL2-VH2-VH3-CL-CH2-CH3, VL3-CH1-VH2-VL2-VH3-CL-CH2-CH3, VH3-CL-VL2-VH2-VL3-CH1-CH2-CH3, VH3-CL-VH2-VL2-VL3-CH1-CH2-CH3, VH3-CH1-VL2-VH2-VL3-CL-CH2-CH3, or VH3-CH1-VH2-VL2-VL3-CL-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-CH2-CH3, VL1-L1-CH1-L2-VH1-L3-CL-CH2-CH3, VH1-L1-CL-L2-VL1- L3-CH1-CH2-CH3, or VH1-L1-CH1-L2-VL1-L3-CL-CH2-CH3; and wherein the second polypeptide has a structure represented by VL2-L4-CL-L5-VL3-L6-VH3-L7-VH2-L8-CH1-CH2-CH3, VL2-L4-CL-L5-VH3-L6-VL3-L7-VH2-L8-CH1-CH2-CH3, VL2-L4-CH1-L5-VL3-L6-VH3-L7-VH2-L8-CL-CH2-CH3, VL2-L4-CH1-L5-VH3-L6-VL3-L7-VH2-L8-CL-CH2-CH3, VH2-L4-CL-L5-VL3-L6-VH3-L7-VL2-L8-CH1-CH2-CH3, VH2-L4-CL-L5-VH3-L6-VL3-L7-VL2-L8-CH1-CH2-CH3, VH2-L4-CH1-L5-VL3-L6-VH3-L7-VL2-L8-CL-CH2-CH3, VH2-L4-CH1-L5-VH3-L6-VL3-L7-VL2-L8-CL-CH2-CH3, VL3-L4-CL-L5-VL2-L6-VH2-L7-VH3-L8-CH1-CH2-CH3, VL3-L4-CL-L5-VH2-L6-VL2-L7-VH3-L8-CH1-CH2-CH3, VL3-L4-CH1-L5-VL2-L6-VH2-L7-VH3-L8-CL-CH2-CH3, VL3-L4-CH1-L5-VH2-L6-VL2-L7-VH3-L8-CL-CH2-CH3, VH3-L4-CL-L5-VL2-L6-VH2-L7-VL3-L8-CH1-CH2-CH3, VH3-L4-CL-L5-VH2-L6-VL2-L7-VL3-L8-CH1-CH2-CH3, VH3-L4-CH1-L5-VL2-L6-VH2-L7-VL3-L8-CL-CH2-CH3, or VH3-L4-CH1-L5-VH2-L6-VL2-L7-VL3-L8-CL-CH2-CH3.

In some aspects, an antigen binding polypeptide complex provided herein comprises a first polypeptide, and a second polypeptide; wherein the first polypeptide has a structure represented by VL1-CL-VH1-CH1, VL1-CH1-VH1-CL, VH1-CL-VL1-CH1, or VH1-CH1-VL1-CL; and wherein the second polypeptide has a structure represented by VL2-VH2-VL3-CL-VH3-CH1, VL2-VH2-VL3-CH1-VH3-CL, VL2-VH2-VH3-CL-VL3-CH1, VL2-VH2-VH3-CH1-VL3-CL, VL2-VL3-VH2-CL-VH3-CH1, VL2-VL3-VH2-CH1-VH3-CL, VL2-VH3-VH2-CL-VL3-CH1, VL2-VH3-VH2-CH1-VL3-CL, VH2-VL2-VL3-CL-VH3-CH1, VH2-VL2-VL3-CH1-VH3-CL, VH2-VL2-VH3-CL-VL3-CH1, VH2-VL2-VH3-CH1-VL3-CL, VH2-VL3-VL2-CL-VH3-CH1, VH2-VL3-VL2-CH1-VH3-CL, VH2-VH3-VL2-CL-VL3-CH1, VH2-VH3-VL2-CH1-VL3-CL, VL3-VL2-VH3-CL-VH2-CH1, VL3-VL2-VH3-CH1-VH2-CL, VL3-VH2-VH3-CL-VL2-CH1, VL3-VH2-VH3-CH1-VL2-CL, VL3-VH3-VL2-CL-VH2-CH1, VL3-VH3-VL2-CH1-VH2-CL, VL3-VH3-VH2-CL-VL2-CH1, VL3-VH3-VH2-CH1-VL2-CL, VH3-VL2-VL3-CL-VH2-CH1, VH3-VL2-VL3-CH1-VH2-CL, VH3-VH2-VL3-CL-VL2-CH1, VH3-VH2-VL3-CH1-VL2-CL, VH3-VL3-VL2-CL-VH2-CH1, VH3-VL3-VL2-CH1-VH2-CL, VH3-VL3-VH2-CL-VL2-CH1, or VH3-VL3-VH2-CH1-VL2-CL.

In some aspects, an antigen binding polypeptide complex provided herein comprises a first polypeptide, and a second polypeptide; wherein the first polypeptide has a structure represented by VL1-L1-CL-L2-VH1-L3-CH1, VL1-L1-CH1-L2-VH1-L3-CL, VH1-L1-CL-L2-VL1-L3-CH1, or VH1-L1- CH1-L2-VL1-L3-CL; and wherein the second polypeptide has a structure represented by VL2-L4-VH2-L5-VL3-L6-CL-L7-VH3-L8-CH1, VL2-L4-VH2-L5-VL3-L6-CH1-L7-VH3-L8-CL, VL2-L4-VH2-L5-VH3-L6-CL-L7-VL3-L8-CH1, VL2-L4-VH2-L5-VH3-L6-CH1-L7-VL3-L8-CL, VL2-L4-VL3-L5-VH2-L6-CL-L7-VH3-L8-CH1, VL2-L4-VL3-L5-VH2-L6-CH1-L7-VH3-L8-CL, VL2-L4-VH3-L5-VH2-L6-CL-L7-VL3-L8-CH1, VL2-L4-VH3-L5-VH2-L6-CH1-L7-VL3-L8-CL, VH2-L4-VL2-L5-VL3-L6-CL-L7-VH3-L8-CH1, VH2-L4-VL2-L5-VL3-L6-CH1-L7-VH3-L8-CL, VH2-L4-VL2-L5-VH3-L6-CL-L7-VL3-L8-CH1, VH2-L4-VL2-L5-VH3-L6-CH1-L7-VL3-L8-CL, VH2-L4-VL3-L5-VL2-L6-CL-L7-VH3-L8-CH1, VH2-L4-VL3-L5-VL2-L6-CH1-L7-VH3-L8-CL, VH2-L4-VH3-L5-VL2-L6-CL-L7-VL3-L8-CH1, VH2-L4-VH3-L5-VL2-L6-CH1-L7-VL3-L8-CL, VL3-L4-VL2-L5-VH3-L6-CL-L7-VH2-L8-CH1, VL3-L4-VL2-L5-VH3-L6-CH1-L7-VH2-L8-CL, VL3-L4-VH2-L5-VH3-L6-CL-L7-VL2-L8-CH1, VL3-L4-VH2-L5-VH3-L6-CH1-L7-VL2-L8-CL, VL3-L4-VH3-L5-VL2-L6-CL-L7-VH2-L8-CH1, VL3-L4-VH3-L5-VL2-L6-CH1-L7-VH2-L8-CL, VL3-L4-VH3-L5-VH2-L6-CL-L7-VL2-L8-CH1, VL3-L4-VH3-L5-VH2-L6-CH1-L7-VL2-L8-CL, VH3-L4-VL2-L5-VL3-L6-CL-L7-VH2-L8-CH1, VH3-L4-VL2-L5-VL3-L6-CH1-L7-VH2-L8-CL, VH3-L4-VH2-L5-VL3-L6-CL-L7-VL2-L8-CH1, VH3-L4-VH2-L5-VL3-L6-CH1-L7-VL2-L8-CL, VH3-L4-VL3-L5-VL2-L6-CL-L7-VH2-L8-CH1, VH3-L4-VL3-L5-VL2-L6-CH1-L7-VH2-L8-CL, VH3-L4-VL3-L5-VH2-L6-CL-L7-VL2-L8-CH1, or VH3-L4-VL3-L5-VH2-L6-CH1-L7-VL2-L8-CL.

In some aspects, an antigen binding polypeptide complex provided herein comprises a first polypeptide, and a second polypeptide; wherein the first polypeptide has a structure represented by VL1-CL-VH1-CH1-CH2-CH3, VL1-CH1-VH1-CL-CH2-CH3, VH1-CL-VL1-CH1-CH2-CH3, or VH1-CH1-VL1-CL-CH2-CH3; and wherein the second polypeptide has a structure represented by VL2-VH2-VL3-CL-VH3-CH1-CH2-CH3, VL2-VH2-VL3-CH1-VH3-CL-CH2-CH3, VL2-VH2-VH3-CL-VL3-CH1-CH2-CH3, VL2-VH2-VH3-CH1-VL3-CL-CH2-CH3, VL2-VL3-VH2-CL-VH3-CH1-CH2-CH3, VL2-VL3-VH2-CH1-VH3-CL-CH2-CH3, VL2-VH3-VH2-CL-VL3-CH1-CH2-CH3, VL2-VH3-VH2-CH1-VL3-CL-CH2-CH3, VH2-VL2-VL3-CL-VH3-CH1-CH2-CH3, VH2-VL2-VL3-CH1-VH3-CL-CH2-CH3, VH2-VL2-VH3-CL-VL3-CH1-CH2-CH3, VH2-VL2-VH3-CH1-VL3-CL-CH2-CH3, VH2-VL3-VL2-CL-VH3-CH1-CH2-CH3, VH2-VL3-VL2-CH1-VH3-CL-CH2-CH3, VH2-VH3-VL2-CL-VL3-CH1-CH2-CH3, VH2-VH3-VL2-CH1-VL3-CL-CH2-CH3, VL3-VL2-VH3-CL-VH2-CH1-CH2-CH3, VL3-VL2-VH3-CH1-VH2-CL-CH2-CH3, VL3-VH2-VH3-CL-VL2-CH1-CH2-CH3, VL3-VH2-VH3-CH1-VL2-CL-CH2-CH3, VL3-VH3-VL2-CL-VH2-CH1-CH2-CH3, VL3-VH3-VL2-CH1-VH2-CL-CH2-CH3, VL3-VH3-VH2-CL-VL2-CH1-CH2-CH3, VL3-VH3-VH2-CH1-VL2-CL-CH2-CH3, VH3-VL2-VL3-CL-VH2-CH1-CH2-CH3, VH3-VL2-VL3-CH1-VH2-CL-CH2-CH3, VH3-VH2-VL3-CL-VL2-CH1-CH2-CH3, VH3-VH2-VL3-CH1-VL2-CL-CH2-CH3, VH3-VL3-VL2-CL-VH2-CH1-CH2-CH3, VH3-VL3-VL2-CH1-VH2-CL-CH2-CH3, VH3-VL3-VH2-CL-VL2-CH1-CH2-CH3, or VH3-VL3-VH2-CH1-VL2-CL-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-CH2-CH3, VL1-L1-CH1-L2-VH1-L3-CL-CH2-CH3, VH1-L1-CL-L2-VL1- L3-CH1-CH2-CH3, or VH1-L1-CH1-L2-VL1-L3-CL-CH2-CH3; and wherein the second polypeptide has a structure represented by VL2-L4-VH2-L5-VL3-L6-CL-L7-VH3-L8-CH1-CH2-CH3, VL2-L4-VH2-L5-VL3-L6-CH1-L7-VH3-L8-CL-CH2-CH3, VL2-L4-VH2-L5-VH3-L6-CL-L7-VL3-L8-CH1-CH2-CH3, VL2-L4-VH2-L5-VH3-L6-CH1-L7-VL3-L8-CL-CH2-CH3, VL2-L4-VL3-L5-VH2-L6-CL-L7-VH3-L8-CH1-CH2-CH3, VL2-L4-VL3-L5-VH2-L6-CH1-L7-VH3-L8-CL-CH2-CH3, VL2-L4-VH3-L5-VH2-L6-CL-L7-VL3-L8-CH1-CH2-CH3, VL2-L4-VH3-L5-VH2-L6-CH1-L7-VL3-L8-CL-CH2-CH3, VH2-L4-VL2-L5-VL3-L6-CL-L7-VH3-L8-CH1-CH2-CH3, VH2-L4-VL2-L5-VL3-L6-CH1-L7-VH3-L8- CL-CH2-CH3, VH2-L4-VL2-L5-VH3-L6-CL-L7-VL3-L8-CH1-CH2-CH3, VH2-L4-VL2-L5-VH3-L6-CH1-L7-VL3-L8-CL-CH2-CH3, VH2-L4-VL3-L5-VL2-L6-CL-L7-VH3-L8-CH1-CH2-CH3, VH2-L4-VL3-L5-VL2-L6-CH1-L7-VH3-L8-CL-CH2-CH3, VH2-L4-VH3-L5-VL2-L6-CL-L7-VL3-L8-CH1-CH2-CH3, VH2-L4-VH3-L5-VL2-L6-CH1-L7-VL3-L8-CL-CH2-CH3, VL3-L4-VL2-L5-VH3-L6-CL-L7-VH2-L8-CH1-CH2-CH3, VL3-L4-VL2-L5-VH3-L6-CH1-L7-VH2-L8-CL-CH2-CH3, VL3-L4-VH2-L5-VH3-L6-CL-L7-VL2-L8-CH1-CH2-CH3, VL3-L4-VH2-L5-VH3-L6-CH1-L7-VL2-L8-CL-CH2-CH3, VL3-L4-VH3-L5-VL2-L6-CL-L7-VH2-L8-CH1-CH2-CH3, VL3-L4-VH3-L5-VL2-L6-CH1-L7-VH2-L8- CL-CH2-CH3, VL3-L4-VH3-L5-VH2-L6-CL-L7-VL2-L8-CH1-CH2-CH3, VL3-L4-VH3-L5-VH2-L6-CH1-L7-VL2-L8-CL-CH2-CH3, VH3-L4-VL2-L5-VL3-L6-CL-L7-VH2-L8-CH1-CH2-CH3, VH3-L4-VL2-L5-VL3-L6-CH1-L7-VH2-L8-CL-CH2-CH3, VH3-L4-VH2-L5-VL3-L6-CL-L7-VL2-L8-CH1-CH2-CH3, VH3-L4-VH2-L5-VL3-L6-CH1-L7-VL2-L8-CL-CH2-CH3, VH3-L4-VL3-L5-VL2-L6-CL-L7-VH2-L8-CH1-CH2-CH3, VH3-L4-VL3-L5-VL2-L6-CH1-L7-VH2-L8-CL-CH2-CH3, VH3-L4-VL3-L5-VH2-L6-CL-L7-VL2-L8-CH1-CH2-CH3, or VH3-L4-VL3-L5-VH2-L6-CH1-L7-VL2-L8-CL-CH2-CH3.

In some aspects, an antigen binding polypeptide complex provided herein comprises a first polypeptide, and a second polypeptide; wherein the first polypeptide has a structure represented by VL1-CL-VH1-CH1, VL1-CH1-VH1-CL, VH1-CL-VL1-CH1, or VH1-CH1-VL1-CL; and wherein the second polypeptide has a structure represented by VL2-CL-VH2-CH1-VL3-VH3, VL2-CL-VH2-CH1-VH3-VL3, VL2-CL-VL3-CH1-VH2-VH3, VL2-CL-VH3-CH1-VH2-VL3, VL2-CH1-VH2-CL-VL3-VH3, VL2-CH1-VH2-CL-VH3-VL3, VL2-CH1-VL3-CL-VH2-VH3, VL2-CH1-VH3-CL-VH2-VL3, VH2-CL-VL2-CH1-VL3-VH3, VH2-CL-VL2-CH1-VH3-VL3, VH2-CL-VL3-CH1-VL2-VH3, VH2-CL-VH3-CH1-VL2-VL3, VH2-CH1-VL2-CL-VL3-VH3, VH2-CH1-VL2-CL-VH3-VL3, VH2-CH1-VL3-CL-VL2-VH3, VH2-CH1-VH3-CL-VL2-VL3, VL3-CL-VL2-CH1-VH3-VH2, VL3-CL-VH2-CH1-VH3-VL2, VL3-CL-VH3-CH1-VL2-VH2, VL3-CL-VH3-CH1-VH2-VL2, VL3-CH1-VL2-CL-VH3-VH2, VL3-CH1-VH2-CL-VH3-VL2, VL3-CH1-VH3-CL-VL2-VH2, VL3-CH1-VH3-CL-VH2-VL2, VH3-CL-VL2-CH1-VL3-VH2, VH3-CL-VH2-CH1-VL3-VL2, VH3-CL-VL3-CH1-VL2-VH2, VH3-CL-VL3-CH1-VH2-VL2, VH3-CH1-VL2-CL-VL3-VH2, VH3-CH1-VH2-CL-VL3-VL2, VH3-CH1-VL3-CL-VL2-VH2, or VH3-CH1-VL3-CL-VH2-VL2.

In some aspects, an antigen binding polypeptide complex provided herein comprises a first polypeptide, and a second polypeptide; wherein the first polypeptide has a structure represented by VL1-L1-CL-L2-VH1-L3-CH1, VL1-L1-CH1-L2-VH1-L3-CL, VH1-L1-CL-L2-VL1-L3-CH1, or VH1-L1- CH1-L2-VL1-L3-CL; and wherein the second polypeptide has a structure represented by VL2-L4-CL-L5-VH2-L6-CH1-L7-VL3-L8-VH3, VL2-L4-CL-L5-VH2-L6-CH1-L7-VH3-L8-VL3, VL2-L4-CL-L5-VL3-L6-CH1-L7-VH2-L8-VH3, VL2-L4-CL-L5-VH3-L6-CH1-L7-VH2-L8-VL3, VL2-L4-CH1-L5-VH2-L6-CL-L7-VL3-L8-VH3, VL2-L4-CH1-L5-VH2-L6-CL-L7-VH3-L8-VL3, VL2-L4-CH1-L5-VL3-L6-CL- L7-VH2-L8-VH3, VL2-L4-CH1-L5-VH3-L6-CL-L7-VH2-L8-VL3, VH2-L4-CL-L5-VL2-L6-CH1-L7-VL3-L8-VH3, VH2-L4-CL-L5-VL2-L6-CH1-L7-VH3-L8-VL3, VH2-L4-CL-L5-VL3-L6-CH1-L7-VL2-L8-VH3, VH2-L4-CL-L5-VH3-L6-CH1-L7-VL2-L8-VL3, VH2-L4-CH1-L5-VL2-L6-CL-L7-VL3-L8-VH3, VH2-L4-CH1-L5-VL2-L6-CL-L7-VH3-L8-VL3, VH2-L4-CH1-L5-VL3-L6-CL-L7-VL2-L8-VH3, VH2-L4-CH1-L5-VH3-L6-CL-L7-VL2-L8-VL3, VL3-L4-CL-L5-VL2-L6-CH1-L7-VH3-L8-VH2, VL3-L4-CL-L5-VH2-L6-CH1-L7-VH3-L8-VL2, VL3-L4-CL-L5-VH3-L6-CH1-L7-VL2-L8-VH2, VL3-L4-CL- L5-VH3-L6-CH1-L7-VH2-L8-VL2, VL3-L4-CH1-L5-VL2-L6-CL-L7-VH3-L8-VH2, VL3-L4-CH1-L5-VH2-L6-CL-L7-VH3-L8-VL2, VL3-L4-CH1-L5-VH3-L6-CL-L7-VL2-L8-VH2, VL3-L4-CH1-L5-VH3-L6-CL-L7-VH2-L8-VL2, VH3-L4-CL-L5-VL2-L6-CH1-L7-VL3-L8-VH2, VH3-L4-CL-L5-VH2-L6-CH1- L7-VL3-L8-VL2, VH3-L4-CL-L5-VL3-L6-CH1-L7-VL2-L8-VH2, VH3-L4-CL-L5-VL3-L6-CH1-L7-VH2-L8-VL2, VH3-L4-CH1-L5-VL2-L6-CL-L7-VL3-L8-VH2, VH3-L4-CH1-L5-VH2-L6-CL-L7-VL3-L8-VL2, VH3-L4-CH1-L5-VL3-L6-CL-L7-VL2-L8-VH2, or VH3-L4-CH1-L5-VL3-L6-CL-L7-VH2-L8-VL2.

In some aspects, an antigen binding polypeptide complex provided herein comprises a first polypeptide, and a second polypeptide; wherein the first polypeptide has a structure represented by VL1-CL-VH1-CH1-CH2-CH3, VL1-CH1-VH1-CL-CH2-CH3, VH1-CL-VL1-CH1-CH2-CH3, or VH1-CH1-VL1-CL-CH2-CH3; and wherein the second polypeptide has a structure represented by VL2-CL-VH2-CH1-VL3-VH3-CH2-CH3, VL2-CL-VH2-CH1-VH3-VL3-CH2-CH3, VL2-CL-VL3-CH1-VH2-VH3-CH2-CH3, VL2-CL-VH3-CH1-VH2-VL3-CH2-CH3, VL2-CH1-VH2-CL-VL3-VH3-CH2-CH3, VL2-CH1-VH2-CL-VH3-VL3-CH2-CH3, VL2-CH1-VL3-CL-VH2-VH3-CH2-CH3, VL2-CH1-VH3-CL-VH2-VL3-CH2-CH3, VH2-CL-VL2-CH1-VL3-VH3-CH2-CH3, VH2-CL-VL2-CH1-VH3-VL3-CH2-CH3, VH2-CL-VL3-CH1-VL2-VH3-CH2-CH3, VH2-CL-VH3-CH1-VL2-VL3-CH2-CH3, VH2-CH1-VL2-CL-VL3-VH3-CH2-CH3, VH2-CH1-VL2-CL-VH3-VL3-CH2-CH3, VH2-CH1-VL3-CL-VL2-VH3-CH2-CH3, VH2-CH1-VH3-CL-VL2-VL3-CH2-CH3, VL3-CL-VL2-CH1-VH3-VH2-CH2-CH3, VL3-CL-VH2-CH1-VH3-VL2-CH2-CH3, VL3-CL-VH3-CH1-VL2-VH2-CH2-CH3, VL3-CL-VH3-CH1-VH2-VL2-CH2-CH3, VL3-CH1-VL2-CL-VH3-VH2-CH2-CH3, VL3-CH1-VH2-CL-VH3-VL2-CH2-CH3, VL3-CH1-VH3-CL-VL2-VH2-CH2-CH3, VL3-CH1-VH3-CL-VH2-VL2-CH2-CH3, VH3-CL-VL2-CH1-VL3-VH2-CH2-CH3, VH3-CL-VH2-CH1-VL3-VL2-CH2-CH3, VH3-CL-VL3-CH1-VL2-VH2-CH2-CH3, VH3-CL-VL3-CH1-VH2-VL2-CH2-CH3, VH3-CH1-VL2-CL-VL3-VH2-CH2-CH3, VH3-CH1-VH2-CL-VL3-VL2-CH2-CH3, VH3-CH1-VL3-CL-VL2-VH2-CH2-CH3, or VH3-CH1-VL3-CL-VH2-VL2-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-CH2-CH3, VL1-L1-CH1-L2-VH1-L3-CL-CH2-CH3, VH1-L1-CL-L2-VL1- L3-CH1-CH2-CH3, or VH1-L1-CH1-L2-VL1-L3-CL-CH2-CH3; and wherein the second polypeptide has a structure represented by VL2-L4-CL-L5-VH2-L6-CH1-L7-VL3-L8-VH3-CH2-CH3, VL2-L4-CL-L5-VH2-L6-CH1-L7-VH3-L8-VL3-CH2-CH3, VL2-L4-CL-L5-VL3-L6-CH1-L7-VH2-L8-VH3-CH2-CH3, VL2-L4-CL-L5-VH3-L6-CH1-L7-VH2-L8-VL3-CH2-CH3, VL2-L4-CH1-L5-VH2-L6-CL-L7-VL3-L8-VH3-CH2-CH3, VL2-L4-CH1-L5-VH2-L6-CL-L7-VH3-L8-VL3-CH2-CH3, VL2-L4-CH1-L5-VL3-L6-CL-L7-VH2-L8-VH3-CH2-CH3, VL2-L4-CH1-L5-VH3-L6-CL-L7-VH2-L8-VL3-CH2-CH3, VH2-L4-CL-L5-VL2-L6-CH1-L7-VL3-L8-VH3-CH2-CH3, VH2-L4-CL-L5-VL2-L6-CH1-L7-VH3-L8-VL3-CH2-CH3, VH2-L4-CL-L5-VL3-L6-CH1-L7-VL2-L8-VH3-CH2-CH3, VH2-L4-CL-L5-VH3-L6-CH1-L7-VL2-L8-VL3-CH2-CH3, VH2-L4-CH1-L5-VL2-L6-CL-L7-VL3-L8-VH3-CH2-CH3, VH2-L4-CH1-L5-VL2-L6-CL-L7-VH3-L8-VL3-CH2-CH3, VH2-L4-CH1-L5-VL3-L6-CL-L7-VL2-L8-VH3-CH2-CH3, VH2-L4-CH1-L5-VH3-L6-CL-L7-VL2-L8-VL3-CH2-CH3, VL3-L4-CL-L5-VL2-L6-CH1-L7-VH3-L8-VH2-CH2-CH3, VL3-L4-CL-L5-VH2-L6-CH1-L7-VH3-L8-VL2-CH2-CH3, VL3-L4-CL-L5-VH3-L6-CH1-L7-VL2-L8-VH2-CH2-CH3, VL3-L4-CL-L5-VH3-L6-CH1-L7-VH2-L8-VL2-CH2-CH3, VL3-L4-CH1-L5-VL2-L6-CL-L7-VH3-L8-VH2-CH2-CH3, VL3-L4-CH1-L5-VH2-L6-CL-L7-VH3-L8-VL2-CH2-CH3, VL3-L4-CH1-L5-VH3-L6-CL-L7-VL2-L8-VH2-CH2-CH3, VL3-L4-CH1-L5-VH3-L6-CL-L7-VH2-L8-VL2-CH2-CH3, VH3-L4-CL-L5-VL2-L6-CH1-L7-VL3-L8-VH2-CH2-CH3, VH3-L4-CL-L5-VH2-L6-CH1-L7-VL3-L8-VL2-CH2-CH3, VH3-L4-CL-L5-VL3-L6-CH1-L7-VL2-L8-VH2-CH2-CH3, VH3-L4-CL-L5-VL3-L6-CH1-L7-VH2-L8-VL2-CH2-CH3, VH3-L4-CH1-L5-VL2-L6-CL-L7-VL3-L8-VH2-CH2-CH3, VH3-L4-CH1-L5-VH2-L6-CL-L7-VL3-L8-VL2-CH2-CH3, VH3-L4-CH1-L5-VL3-L6-CL-L7-VL2-L8-VH2-CH2-CH3, or VH3-L4-CH1-L5-VL3-L6-CL-L7-VH2-L8-VL2-CH2-CH3.

In some aspects, VL1 is a first immunoglobulin light chain variable region that specifically binds to a SARS-CoV-2 protein (e.g., a SARS-CoV-2 spike protein); VL2 is a second immunoglobulin light chain variable region that specifically binds to a SARS-CoV-2 protein (e.g., a SARS-CoV-2 spike protein); VL3 is a third immunoglobulin light chain variable region that specifically binds to a SARS-CoV-2 protein (e.g., a SARS-CoV-2 spike protein); VH1 is a first immunoglobulin heavy chain variable region that specifically binds to a SARS-CoV-2 protein (e.g., a SARS-CoV-2 spike protein); VH2 is a second immunoglobulin heavy chain variable region that specifically binds to a SARS-CoV-2 protein (e.g., a SARS-CoV-2 spike protein); VH3 is a third immunoglobulin heavy chain variable region that specifically binds to a SARS-CoV-2 protein (e.g., a SARS-CoV-2 spike protein); CH1 is an immunoglobulin heavy chain constant region 1; CL is an immunoglobulin light chain constant region; and L1-L8 are optional amino acid linkers. In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the VL1 and the VL2 each comprise a CDR1, a CDR2, and a CDR3 that are the same. In some aspects, the VL1 and the VL2 each comprise a CDR1, a CDR2, and a CDR3 that are different. In some aspects, the VL1 and the VL3 each comprise a CDR1, a CDR2, and a CDR3 that are the same. In some aspects, the VL1 and the VL3 each comprise a CDR1, a CDR2, and a CDR3 that are different. In some aspects, the VL2 and the VL3 each comprise a CDR1, a CDR2, and a CDR3 that are the same. In some aspects, the VL2 and the VL3 each comprise a CDR1, a CDR2, and a CDR3 that are different.

In some aspects, the VL1 and the VL2 each comprise an amino acid sequence, wherein the amino acid sequence comprised in VL1 and the amino acid sequence comprised in VL2 are the same. In some aspects, the VL1 and the VL2 each comprise an amino acid sequence, wherein the amino acid sequence comprised in VL1 and the amino acid sequence comprised in VL2 are different. In some aspects, the VL1 and the VL3 each comprise an amino acid sequence, wherein the amino acid sequence comprised in VL1 and the amino acid sequence comprised in VL3 are the same. In some aspects, the VL1 and the VL3 each comprise an amino acid sequence, wherein the amino acid sequence comprised in VL1 and the amino acid sequence comprised in VL3 are different. In some aspects, the VL2 and the VL3 each comprise an amino acid sequence, wherein the amino acid sequence comprised in VL2 and the amino acid sequence comprised in VL3 are the same. In some aspects, the VL2 and the VL3 each comprise an amino acid sequence, wherein the amino acid sequence comprised in VL2 and the amino acid sequence comprised in VL3 are different.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL3 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL3 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL3 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL3 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061 or an amino acid sequence at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL3 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL3 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325. In some aspects, VL3 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL3 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH3 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH3 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH3 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH3 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068. In some aspects, VH3 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH3 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, VH3 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH3 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, at least one of VL1, VL2, or VL3 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1, VH2, or VH3 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1, VL2, or VL3 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1, VH2, or VH3 comprised in the antigen binding polypeptide complexes provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1, VL2, or VL3 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1, VH2, or VH3 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1, VL2, or VL3 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1, VH2, or VH3 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL3 comprising (xiii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (xiv) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (xv) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH3 comprising (xvi) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xvii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xviii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (v) a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851.860, 867, 873, 880, and 988; (vi) and a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; a VL3 comprising (xiii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (xiv) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (xv) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH3 comprising (xvi) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xvii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xviii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (v) a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (vi) and a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL1 comprises an amino acid sequence at least 65%, at least 70%, at least 75%, at least 80%, at least 85%, at least 90%, at least 95%, at least 99%, or 100% identical to SEQ ID NO: 11, and VH1 comprises an amino acid sequence at least 65%, at least 70%, at least 75%, at least 80%, at least 85%, at least 90%, at least 95%, at least 99%, or 100% identical to SEQ ID NO: 14.

In some aspects, VL2 comprises an amino acid sequence at least 65%, at least 70%, at least 75%, at least 80%, at least 85%, at least 90%, at least 95%, at least 99%, or 100% identical to SEQ ID NO: 17, and VH2 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, VL2 comprises an amino acid sequence at least 65%, at least 70%, at least 75%, at least 80%, at least 85%, at least 90%, at least 95%, at least 99%, or 100% identical to SEQ ID NO: 25, and VH2 comprises an amino acid sequence at least 65%, at least 70%, at least 75%, at least 80%, at least 85%, at least 90%, at least 95%, at least 99%, or 100% identical to SEQ ID NO: 29.

In some aspects, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, VL3 comprises an amino acid sequence at least 65%, at least 70%, at least 75%, at least 80%, at least 85%, at least 90%, at least 95%, at least 99%, or 100% identical to SEQ ID NO: 25, and VH3 comprises an amino acid sequence at least 65%, at least 70%, at least 75%, at least 80%, at least 85%, at least 90%, at least 95%, at least 99%, or 100% identical to SEQ ID NO: 29.

In some aspects, the first polypeptide comprises a structure represented by VL1-CL-VH1-CH1, VL1-L1-CL-L2-VH1-L3-CH1, VL1-CL-VH1-CH1-CH2-CH3, or VL1-L1-CL-L2-VH1-L3-CH1-CH2-CH3; and the second polypeptide comprises a structure represented by VL2-CL-VL3-VH3-VH2-CH1, VL2-L4-CL-L5-VL3-L6-VH3-L7-VH2-L8-CH1, VL2-CL-VL3-VH3-VH2-CH1-CH2-CH3, or VL2-L4-CL-L5-VL3-L6-VH3-L7-VH2-L8-CH1-CH2-CH3, wherein VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 17, VH2 comprises an amino acid sequence at least 65%, at least 70%, at least 75%, at least 80%, at least 85%, at least 90%, at least 95%, at least 99%, or 100% identical to SEQ ID NO: 21 or 22, VL3 comprises an amino acid sequence at least 65%, at least 70%, at least 75%, at least 80%, at least 85%, at least 90%, at least 95%, at least 99%, or 100% identical to SEQ ID NO: 25 and VH3 comprises an amino acid sequence at least 65%, at least 70%, at least 75%, at least 80%, at least 85%, at least 90%, at least 95%, at least 99%, or 100% identical to SEQ ID NO: 29.

In some aspects, the first polypeptide comprises a structure represented by VL1-CL-VH1-CH1, VL1-L1-CL-L2-VH1-L3-CH1, VL1-CL-VH1-CH1-CH2-CH3, or VL1-L1-CL-L2-VH1-L3-CH1-CH2-CH3; and the second polypeptide comprises a structure represented by VL2-CL-VL3-VH3-VH2-CH1, VL2-L4-CL-L5-VL3-L6-VH3-L7-VH2-L8-CH1, VL2-CL-VL3-VH3-VH2-CH1-CH2-CH3, or VL2-L4-CL-L5-VL3-L6-VH3-L7-VH2-L8-CH1-CH2-CH3, wherein VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, the first polypeptide comprises a structure represented by VL1-CL-VH1-CH1, VL1-L1-CL-L2-VH1-L3-CH1, VL1-CL-VH1-CH1-CH2-CH3, or VL1-L1-CL-L2-VH1-L3-CH1-CH2-CH3; and the second polypeptide comprises a structure represented by VL2-VH2-VL3-CL-VH3-CH1, VL2-L4-VH2-L5-VL3-L6-CL-L7-VH3-L8-CH1, VL2-VH2-VL3-CL-VH3-CH1-CH2-CH3, or VL2-L4-VH2-L5-VL3-L6-CL-L7-VH3-L8-CH1-CH2-CH3, wherein VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, the first polypeptide comprises a structure represented by VL1-CL-VH1-CH1, VL1-L1-CL-L2-VH1-L3-CH1, VL1-CL-VH1-CH1-CH2-CH3, or VL1-L1-CL-L2-VH1-L3-CH1-CH2-CH3; and the second polypeptide comprises a structure represented by VL3-CL-VH3-CH1-VL4-VH4, VL3-L4-CL-L5-VH3-L6-CH1-L7-VL4-L8-VH4, VL3-CL-VH3-CH1-VL4-VH4-CH2-CH3, or VL3-L4-CL-L5-VH3-L6-CH1-L7-VL4-L8-VH4-CH2-CH3, wherein VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 445 and the second polypeptide comprises an amino acid sequence at least 65%, at least 70%, at least 75%, at least 80%, at least 85%, at least 90%, at least 95%, at least 99%, or 100% identical to SEQ ID NO: 446.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 567 and the second polypeptide is encoded by a nucleotide sequence at least 65%, at least 70%, at least 75%, at least 80%, at least 85%, at least 90%, at least 95%, at least 99%, or 100% identical to SEQ ID NO: 568.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 447 and the second polypeptide comprises an amino acid sequence at least 65%, at least 70%, at least 75%, at least 80%, at least 85%, at least 90%, at least 95%, at least 99%, or 100% identical to SEQ ID NO: 448.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 569 and the second polypeptide is encoded by a nucleotide sequence at least 65%, at least 70%, at least 75%, at least 80%, at least 85%, at least 90%, at least 95%, at least 99%, or 100% identical to SEQ ID NO: 570.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 449 and the second polypeptide comprises an amino acid sequence at least 65%, at least 70%, at least 75%, at least 80%, at least 85%, at least 90%, at least 95%, at least 99%, or 100% identical to SEQ ID NO: 450.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 571 and the second polypeptide is encoded by a nucleotide sequence at least 65%, at least 70%, at least 75%, at least 80%, at least 85%, at least 90%, at least 95%, at least 99%, or 100% identical to SEQ ID NO: 572.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 451 and the second polypeptide comprises an amino acid sequence at least 65%, at least 70%, at least 75%, at least 80%, at least 85%, at least 90%, at least 95%, at least 99%, or 100% identical to SEQ ID NO: 452.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 573 and the second polypeptide is encoded by a nucleotide sequence at least 65%, at least 70%, at least 75%, at least 80%, at least 85%, at least 90%, at least 95%, at least 99%, or 100% identical to SEQ ID NO: 574.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOS: 635.636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOS: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof. In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, an antigen binding polypeptide complex provided herein comprises a first polypeptide, and a second polypeptide; wherein the first polypeptide has a structure represented by VL1-CL-VL2-VH2-VH1-CH1, VL1-CL-VH2-VL2-VH1-CH1, VL1-CH1-VL2-VH2-VH1-CL, VL1-CH1-VH2-VL2-VH1-CL, VH1-CL-VL2-VH2-VL1-CH1, VH1-CL-VH2-VL2-VL1-CH1, VH1-CH1-VL2-VH2-VL1-CL, VH1-CH1-VH2-VL2-VL1-CL, VL2-CL-VL1-VH1-VH2-CH1, VL2-CL-VH1-VL1-VH2-CH1, VL2-CH1-VL1-VH1-VH2-CL, VL2-CH1-VH1-VL1-VH2-CL, VH2-CL-VL1-VH1-VL2-CH1, VH2-CL-VH1-VL1-VL2-CH1, VH2-CH1-VL1-VH1-VL2-CL, or VH2-CH1-VH1-VL1-VL2-CL; and wherein the second polypeptide has a structure represented by VL3-CL-VL4-VH4-VH3-CH1, VL3-CL-VH4-VL4-VH3-CH1, VL3-CH1-VL4-VH4-VH3-CL, VL3-CH1-VH4-VL4-VH3-CL, VH3-CL-VL4-VH4-VL3-CH1, VH3-CL-VH4-VL4-VL3-CH1, VH3-CH1-VL4-VH4-VL3-CL, VH3-CH1-VH4-VL4-VL3-CL, VL4-CL-VL3-VH3-VH4-CH1, VL4-CL-VH3-VL3-VH4-CH1, VL4-CH1-VL3-VH3-VH4-CL, VL4-CH1-VH3-VL3-VH4-CL, VH4-CL-VL3-VH3-VL4-CH1, VH4-CL-VH3-VL3-VL4-CH1, VH4-CH1-VL3-VH3-VL4-CL, or VH4-CH1-VH3-VL3-VL4-CL.

In some aspects, an antigen binding polypeptide complex provided herein comprises a first polypeptide, and a second polypeptide; wherein the first polypeptide has a structure represented by VL1-L1-CL-L2-VL2-L3-VH2-L4-VH1-L5-CH1, VL1-L1-CL-L2-VH2-L3-VL2-L4-VH1-L5-CH1, VL1-L1-CH1-L2-VL2-L3-VH2-L4-VH1-L5-CL, VL1-L1-CH1-L2-VH2-L3-VL2-L4-VH1-L5-CL, VH1-L1-CL-L2-VL2-L3-VH2-L4-VL1-L5-CH1, VH1-L1-CL-L2-VH2-L3-VL2-L4-VL1-L5-CH1, VH1-L1-CH1-L2-VL2-L3-VH2-L4-VL1-L5-CL, VH1-L1-CH1-L2-VH2-L3-VL2-L4-VL1-L5-CL, VL2-L1-CL-L2-VL1-L3-VH1-L4-VH2-L5-CH1, VL2-L1-CL-L2-VH1-L3-VL1-L4-VH2-L5-CH1, VL2-L1-CH1-L2-VL1-L3-VH1-L4-VH2-L5-CL, VL2-L1-CH1-L2-VH1-L3-VL1-L4-VH2-L5-CL, VH2-L1-CL-L2-VL1-L3-VH1-L4-VL2-L5-CH1, VH2-L1-CL-L2-VH1-L3-VL1-L4-VL2-L5-CH1, VH2-L1-CH1-L2-VL1-L3-VH1-L4- VL2-L5-CL, or VH2-L1-CH1-L2-VH1-L3-VL1-L4-VL2-L5-CL; and wherein the second polypeptide has a structure represented by VL3-L6-CL-L7-VL4-L8-VH4-L9-VH3-L10-CH1, VL3-L6-CL-L7-VH4-L8-VL4-L9-VH3-L10-CH1, VL3-L6-CH1-L7-VL4-L8-VH4-L9-VH3-L10-CL, VL3-L6-CH1-L7-VH4-L8-VL4-L9-VH3-L10-CL, VH3-L6-CL-L7-VL4-L8-VH4-L9-VL3-L10-CH1, VH3-L6-CL-L7-VH4-L8-VL4-L9- VL3-L10-CH1, VH3-L6-CH1-L7-VL4-L8-VH4-L9-VL3-L10-CL, VH3-L6-CH1-L7-VH4-L8-VL4-L9-VL3-L10-CL, VL4-L6-CL-L7-VL3-L8-VH3-L9-VH4-L10-CH1, VL4-L6-CL-L7-VH3-L8-VL3-L9-VH4- L10-CH1, VL4-L6-CH1-L7-VL3-L8-VH3-L9-VH4-L10-CL, VL4-L6-CH1-L7-VH3-L8-VL3-L9-VH4-L10-CL, VH4-L6-CL-L7-VL3-L8-VH3-L9-VL4-L10-CH1, VH4-L6-CL-L7-VH3-L8-VL3-L9-VL4-L10- CH1, VH4-L6-CH1-L7-VL3-L8-VH3-L9-VL4-L10-CL, or VH4-L6-CH1-L7-VH3-L8-VL3-L9-VL4-L10-CL.

In some aspects, an antigen binding polypeptide complex provided herein comprises a first polypeptide, and a second polypeptide; wherein the first polypeptide has a structure represented by VL1-CL-L1-VL2-L2-VH2-L3-VH1-CH1; VL1-CL-L1-VH2-L2-VL2-L3-VH1-CH1; VL1-CH1-L1-VL2-L2-VH2-L3-VH1-CL; VL1-CH1-L1-VH2-L2-VL2-L3-VH1-CL; VH1-CL-L1-VL2-L2-VH2-L3-VL1-CH1; VH1-CL-L1-VH2-L2-VL2-L3-VL1-CH1; VH1-CH1-L1-VL2-L2-VH2-L3-VL1-CL; VH1-CH1-L1-VH2-L2-VL2-L3-VL1-CL; VL2-CL-L1-VL1-L2-VH1-L3-VH2-CH1; VL2-CL-L1-VH1-L2-VL1-L3-VH2- CH1; VL2-CH1-L1-VL1-L2-VH1-L3-VH2-CL; VL2-CH1-L1-VH1-L2-VL1-L3-VH2-CL; VH2-CL-L1-VL1-L2-VH1-L3-VL2-CH1; VH2-CL-L1-VH1-L2-VL1-L3-VL2-CH1; VH2-CH1-L1-VL1-L2-VH1-L3-VL2-CL; or VH2-CH1-L1-VH1-L2-VL1-L3-VL2-CL; and wherein the second polypeptide has a structure represented by VL3-CL-L4-VL4-L5-VH4-L6-VH3-CH1; VL3-CL-L4-VH4-L5-VL4-L6-VH3-CH1; VL3-CH1-L4-VL4-L5-VH4-L6-VH3-CL; VL3-CH1-L4-VH4-L5-VL4-L6-VH3-CL; VH3-CL-L4-VL4-L5-VH4-L6-VL3-CH1; VH3-CL-L4-VH4-L5-VL4-L6-VL3-CH1; VH3-CH1-L4-VL4-L5-VH4-L6-VL3-CL; VH3-CH1-L4-VH4-L5-VL4-L6-VL3-CL; VL4-CL-L4-VL3-L5-VH3-L6-VH4-CH1; VL4-CL-L4-VH3-L5-VL3-L6-VH4-CH1; VL4-CH1-L4-VL3-L5-VH3-L6-VH4-CL; VL4-CH1-L4-VH3-L5-VL3-L6-VH4-CL; VH4-CL-L4-VL3-L5-VH3-L6-VL4-CH1; VH4-CL-L4-VH3-L5-VL3-L6-VL4-CH1; VH4-CH1-L4-VL3-L5-VH3-L6-VL4-CL; or VH4-CH1-L4-VH3-L5-VL3-L6-VL4-CL.

In some aspects, an antigen binding polypeptide complex provided herein comprises a first polypeptide, and a second polypeptide; wherein the first polypeptide has a structure represented by VL1-CL-VL2-VH2-VH1-CH1-CH2-CH3, VL1-CL-VH2-VL2-VH1-CH1-CH2-CH3, VL1-CH1-VL2-VH2-VH1-CL-CH2-CH3, VL1-CH1-VH2-VL2-VH1-CL-CH2-CH3, VH1-CL-VL2-VH2-VL1-CH1-CH2-CH3, VH1-CL-VH2-VL2-VL1-CH1-CH2-CH3, VH1-CH1-VL2-VH2-VL1-CL-CH2-CH3, VH1-CH1-VH2-VL2-VL1-CL-CH2-CH3, VL2-CL-VL1-VH1-VH2-CH1-CH2-CH3, VL2-CL-VH1-VL1-VH2-CH1-CH2-CH3, VL2-CH1-VL1-VH1-VH2-CL-CH2-CH3, VL2-CH1-VH1-VL1-VH2-CL-CH2-CH3, VH2-CL-VL1-VH1-VL2-CH1-CH2-CH3, VH2-CL-VH1-VL1-VL2-CH1-CH2-CH3, VH2-CH1-VL1-VH1-VL2-CL-CH2-CH3, or VH2-CH1-VH1-VL1-VL2-CL-CH2-CH3; and wherein the second polypeptide has a structure represented by VL3-CL-VL4-VH4-VH3-CH1-CH2-CH3, VL3-CL-VH4-VL4-VH3-CH1-CH2-CH3, VL3-CH1-VL4-VH4-VH3-CL-CH2-CH3, VL3-CH1-VH4-VL4-VH3-CL-CH2-CH3, VH3-CL-VL4-VH4-VL3-CH1-CH2-CH3, VH3-CL-VH4-VL4-VL3-CH1-CH2-CH3, VH3-CH1-VL4-VH4-VL3-CL-CH2-CH3, VH3-CH1-VH4-VL4-VL3-CL-CH2-CH3, VL4-CL-VL3-VH3-VH4-CH1-CH2-CH3, VL4-CL-VH3-VL3-VH4-CH1-CH2-CH3, VL4-CH1-VL3-VH3-VH4-CL-CH2-CH3, VL4-CH1-VH3-VL3-VH4-CL-CH2-CH3, VH4-CL-VL3-VH3-VL4-CH1-CH2-CH3, VH4-CL-VH3-VL3-VL4-CH1-CH2-CH3, VH4-CH1-VL3-VH3-VL4-CL-CH2-CH3, or VH4-CH1-VH3-VL3-VL4-CL-CH2-CH3.

In some aspects, an antigen binding polypeptide complex provided herein comprises a first polypeptide, and a second polypeptide; wherein the first polypeptide has a structure represented by VL1-L1-CL-L2-VL2-L3-VH2-L4-VH1-L5-CH1-CH2-CH3, VL1-L1-CL-L2-VH2-L3-VL2-L4-VH1-L5-CH1-CH2-CH3, VL1-L1-CH1-L2-VL2-L3-VH2-L4-VH1-L5-CL-CH2-CH3, VL1-L1-CH1-L2-VH2-L3-VL2-L4-VH1-L5-CL-CH2-CH3, VH1-L1-CL-L2-VL2-L3-VH2-L4-VL1-L5-CH1-CH2-CH3, VH1-L1-CL- L2-VH2-L3-VL2-L4-VL1-L5-CH1-CH2-CH3, VH1-L1-CH1-L2-VL2-L3-VH2-L4-VL1-L5-CL-CH2-CH3, VH1-L1-CH1-L2-VH2-L3-VL2-L4-VL1-L5-CL-CH2-CH3, VL2-L1-CL-L2-VL1-L3-VH1-L4-VH2-L5-CH1-CH2-CH3, VL2-L1-CL-L2-VH1-L3-VL1-L4-VH2-L5-CH1-CH2-CH3, VL2-L1-CH1-L2-VL1-L3-VH1-L4-VH2-L5-CL-CH2-CH3, VL2-L1-CH1-L2-VH1-L3-VL1-L4-VH2-L5-CL-CH2-CH3, VH2-L1-CL-L2-VL1-L3-VH1-L4-VL2-L5-CH1-CH2-CH3, VH2-L1-CL-L2-VH1-L3-VL1-L4-VL2-L5-CH1-CH2-CH3, VH2-L1-CH1-L2-VL1-L3-VH1-L4-VL2-L5-CL-CH2-CH3, or VH2-L1-CH1-L2-VH1-L3-VL1-L4-VL2-L5-CL-CH2-CH3; and wherein the second polypeptide has a structure represented by VL3-L6-CL-L7-VL4-L8-VH4-L9-VH3-L10-CH1-CH2-CH3, VL3-L6-CL-L7-VH4-L8-VL4-L9-VH3-L10-CH1-CH2-CH3, VL3-L6-CH1-L7-VL4-L8-VH4-L9-VH3-L10-CL-CH2-CH3, VL3-L6-CH1-L7-VH4-L8-VL4-L9- VH3-L10-CL-CH2-CH3, VH3-L6-CL-L7-VL4-L8-VH4-L9-VL3-L10-CH1-CH2-CH3, VH3-L6-CL-L7-VH4-L8-VL4-L9-VL3-L10-CH1-CH2-CH3, VH3-L6-CH1-L7-VL4-L8-VH4-L9-VL3-L10-CL-CH2-CH3, VH3-L6-CH1-L7-VH4-L8-VL4-L9-VL3-L10-CL-CH2-CH3, VL4-L6-CL-L7-VL3-L8-VH3-L9-VH4-L10-CH1-CH2-CH3, VL4-L6-CL-L7-VH3-L8-VL3-L9-VH4-L10-CH1-CH2-CH3, VL4-L6-CH1-L7-VL3-L8-VH3-L9-VH4-L10-CL-CH2-CH3, VL4-L6-CH1-L7-VH3-L8-VL3-L9-VH4-L10-CL-CH2-CH3, VH4-L6-CL- L7-VL3-L8-VH3-L9-VL4-L10-CH1-CH2-CH3, VH4-L6-CL-L7-VH3-L8-VL3-L9-VL4-L10-CH1- CH2-CH3, VH4-L6-CH1-L7-VL3-L8-VH3-L9-VL4-L10-CL-CH2-CH3, or VH4-L6-CH1-L7-VH3-L8-VL3-L9-VL4-L10-CL-CH2-CH3.

In some aspects, an antigen binding polypeptide complex provided herein comprises a first polypeptide, and a second polypeptide; wherein the first polypeptide has a structure represented by VL1-CL-L1-VL2-L2-VH2-L3-VH1-CH1-CH2-CH3; VL1-CL-L1-VH2-L2-VL2-L3-VH1-CH1-CH2-CH3; VL1-CH1-L1-VL2-L2-VH2-L3-VH1-CL-CH2-CH3; VL1-CH1-L1-VH2-L2-VL2-L3-VH1-CL-CH2-CH3; VH1-CL-L1-VL2-L2-VH2-L3-VL1-CH1-CH2-CH3; VH1-CL-L1-VH2-L2-VL2-L3-VL1-CH1-CH2-CH3; VH1-CH1-L1-VL2-L2-VH2-L3-VL1-CL-CH2-CH3; VH1-CH1-L1-VH2-L2-VL2-L3-VL1-CL-CH2-CH3; VL2-CL-L1-VL1-L2-VH1-L3-VH2-CH1-CH2-CH3; VL2-CL-L1-VH1-L2-VL1-L3-VH2-CH1-CH2-CH3; VL2-CH1-L1-VL1-L2-VH1-L3-VH2-CL-CH2-CH3; VL2-CH1-L1-VH1-L2-VL1-L3-VH2-CL-CH2-CH3; VH2-CL-L1-VL1-L2-VH1-L3-VL2-CH1-CH2-CH3; VH2-CL-L1-VH1-L2-VL1-L3-VL2-CH1-CH2-CH3; VH2-CH1-L1-VL1-L2-VH1-L3-VL2-CL-CH2-CH3; or VH2-CH1-L1-VH1-L2-VL1-L3-VL2-CL-CH2-CH3; and wherein the second polypeptide has a structure represented by VL3-CL-L4-VL4-L5-VH4-L6-VH3-CH1-CH2-CH3; VL3-CL-L4-VH4-L5-VL4-L6-VH3-CH1-CH2-CH3; VL3-CH1-L4-VL4-L5-VH4-L6-VH3-CL-CH2-CH3; VL3-CH1-L4-VH4-L5-VL4-L6-VH3-CL-CH2-CH3; VH3-CL-L4-VL4-L5-VH4-L6-VL3-CH1-CH2-CH3; VH3-CL-L4-VH4-L5-VL4-L6-VL3-CH1-CH2-CH3; VH3-CH1-L4-VL4-L5-VH4-L6-VL3-CL-CH2-CH3; VH3-CH1-L4-VH4-L5-VL4-L6-VL3-CL-CH2-CH3; VL4-CL-L4-VL3-L5-VH3-L6-VH4-CH1-CH2-CH3; VL4-CL-L4-VH3-L5-VL3-L6-VH4-CH1-CH2-CH3; VL4-CH1-L4-VL3-L5-VH3-L6-VH4-CL-CH2-CH3; VL4-CH1-L4-VH3-L5-VL3-L6-VH4-CL-CH2-CH3; VH4-CL-L4-VL3-L5-VH3-L6-VL4-CH1-CH2-CH3; VH4-CL-L4-VH3-L5-VL3-L6-VL4-CH1-CH2-CH3; VH4-CH1-L4-VL3-L5-VH3-L6-VL4-CL-CH2-CH3; or VH4-CH1-L4-VH3-L5-VL3-L6-VL4-CL-CH2-CH3.

In some aspects, an antigen binding polypeptide complex provided herein comprises a first polypeptide, and a second polypeptide; wherein the first polypeptide has a structure represented by VL1-VH1-VL2-CL-VH2-CH1, VL1-VH1-VL2-CH1-VH2-CL, VL1-VH1-VH2-CL-VL2-CH1, VL1-VH1-VH2-CH1-VL2-CL, VL1-VL2-VH1-CL-VH2-CH1, VL1-VL2-VH1-CH1-VH2-CL, VL1-VH2-VH1-CL-VL2-CH1, VL1-VH2-VH1-CH1-VL2-CL, VH1-VL1-VL2-CL-VH2-CH1, VH1-VL1-VL2-CH1-VH2-CL, VH1-VL1-VH2-CL-VL2-CH1, VH1-VL1-VH2-CH1-VL2-CL, VH1-VL2-VL1-CL-VH2-CH1, VH1-VL2-VL1-CH1-VH2-CL, VH1-VH2-VL1-CL-VL2-CH1, VH1-VH2-VL1-CH1-VL2-CL, VL2-VL1-VH2-CL-VH1-CH1, VL2-VL1-VH2-CH1-VH1-CL, VL2-VH1-VH2-CL-VL1-CH1, VL2-VH1-VH2-CH1-VL1-CL, VL2-VH2-VL1-CL-VH1-CH1, VL2-VH2-VL1-CH1-VH1-CL, VL2-VH2-VH1-CL-VL1-CH1, VL2-VH2-VH1-CH1-VL1-CL, VH2-VL1-VL2-CL-VH1-CH1, VH2-VL1-VL2-CH1-VH1-CL, VH2-VH1-VL2-CL-VL1-CH1, VH2-VH1-VL2-CH1-VL1-CL, VH2-VL2-VL1-CL-VH1-CH1, VH2-VL2-VL1-CH1-VH1-CL, VH2-VL2-VH1-CL-VL1-CH1, or VH2-VL2-VH1-CH1-VL1-CL; and wherein the second polypeptide has a structure represented by VL3-VH3-VL4-CL-VH4-CH1, VL3-VH3-VL4-CH1-VH4-CL, VL3-VH3-VH4-CL-VL4-CH1, VL3-VH3-VH4-CH1-VL4-CL, VL3-VL4-VH3-CL-VH4-CH1, VL3-VL4-VH3-CH1-VH4-CL, VL3-VH4-VH3-CL-VL4-CH1, VL3-VH4-VH3-CH1-VL4-CL, VH3-VL3-VL4-CL-VH4-CH1, VH3-VL3-VL4-CH1-VH4-CL, VH3-VL3-VH4-CL-VL4-CH1, VH3-VL3-VH4-CH1-VL4-CL, VH3-VL4-VL3-CL-VH4-CH1, VH3-VL4-VL3-CH1-VH4-CL, VH3-VH4-VL3-CL-VL4-CH1, VH3-VH4-VL3-CH1-VL4-CL, VL4-VL3-VH4-CL-VH3-CH1, VL4-VL3-VH4-CH1-VH3-CL, VL4-VH3-VH4-CL-VL3-CH1, VL4-VH3-VH4-CH1-VL3-CL, VL4-VH4-VL3-CL-VH3-CH1, VL4-VH4-VL3-CH1-VH3-CL, VL4-VH4-VH3-CL-VL3-CH1, VL4-VH4-VH3-CH1-VL3-CL, VH4-VL3-VL4-CL-VH3-CH1, VH4-VL3-VL4-CH1-VH3-CL, VH4-VH3-VL4-CL-VL3-CH1, VH4-VH3-VL4-CH1-VL3-CL, VH4-VL4-VL3-CL-VH3-CH1, VH4-VL4-VL3-CH1-VH3-CL, VH4-VL4-VH3-CL-VL3-CH1, or VH4-VL4-VH3-CH1-VL3-CL.

In some aspects, an antigen binding polypeptide complex provided herein comprises a first polypeptide, and a second polypeptide; wherein the first polypeptide has a structure represented by VL1-L1-VH1-L2-VL2-L3-CL-L4-VH2-L5-CH1, VL1-L1-VH1-L2-VL2-L3-CH1-L4-VH2-L5-CL, VL1-L1-VH1-L2-VH2-L3-CL-L4-VL2-L5-CH1, VL1-L1-VH1-L2-VH2-L3-CH1-L4-VL2-L5-CL, VL1-L1-VL2-L2-VH1-L3-CL-L4-VH2-L5-CH1, VL1-L1-VL2-L2-VH1-L3-CH1-L4-VH2-L5-CL, VL1-L1-VH2-L2-VH1-L3-CL-L4-VL2-L5-CH1, VL1-L1-VH2-L2-VH1-L3-CH1-L4-VL2-L5-CL, VH1-L1-VL1-L2-VL2-L3-CL-L4-VH2-L5-CH1, VH1-L1-VL1-L2-VL2-L3-CH1-L4-VH2-L5-CL, VH1-L1-VL1-L2-VH2-L3-CL-L4-VL2-L5-CH1, VH1-L1-VL1-L2-VH2-L3-CH1-L4-VL2-L5-CL, VH1-L1-VL2-L2-VL1-L3-CL-L4-VH2-L5-CH1, VH1-L1-VL2-L2-VL1-L3-CH1-L4-VH2-L5-CL, VH1-L1-VH2-L2-VL1-L3-CL-L4-VL2-L5-CH1, VH1-L1-VH2-L2-VL1-L3-CH1-L4-VL2-L5-CL, VL2-L1-VL1-L2-VH2-L3-CL-L4-VH1-L5-CH1, VL2-L1-VL1-L2-VH2-L3-CH1-L4-VH1-L5-CL, VL2-L1-VH1-L2-VH2-L3-CL-L4-VL1-L5-CH1, VL2-L1-VH1-L2-VH2-L3-CH1-L4-VL1-L5-CL, VL2-L1-VH2-L2-VL1-L3-CL-L4-VH1-L5-CH1, VL2-L1-VH2-L2-VL1-L3-CH1-L4-VH1-L5-CL, VL2-L1-VH2-L2-VH1-L3-CL-L4-VL1-L5-CH1, VL2-L1-VH2-L2-VH1-L3-CH1-L4-VL1-L5-CL, VH2-L1-VL1-L2-VL2-L3-CL-L4-VH1-L5-CH1, VH2-L1-VL1-L2-VL2-L3-CH1-L4-VH1-L5-CL, VH2-L1-VH1-L2-VL2-L3-CL-L4-VL1-L5-CH1, VH2-L1-VH1-L2-VL2-L3-CH1-L4-VL1-L5-CL, VH2-L1-VL2-L2-VL1-L3-CL-L4-VH1-L5-CH1, VH2-L1-VL2-L2-VL1-L3-CH1-L4-VH1-L5-CL, VH2-L1-VL2-L2-VH1-L3-CL-L4-VL1-L5-CH1, or VH2-L1-VL2-L2-VH1-L3-CH1-L4-VL1-L5-CL; and wherein the second polypeptide has a structure represented by VL3-L6-VH3-L7-VL4-L8-CL-L9-VH4-L10-CH1, VL3-L6-VH3-L7-VL4-L8-CH1-L9-VH4-L10-CL, VL3-L6-VH3-L7-VH4-L8-CL-L9-VL4-L10-CH1, VL3-L6-VH3-L7-VH4-L8-CH1-L9-VL4-L10-CL, VL3-L6-VL4-L7-VH3-L8-CL-L9-VH4-L10-CH1, VL3-L6-VL4-L7-VH3-L8-CH1-L9-VH4-L10-CL, VL3-L6-VH4-L7-VH3-L8-CL-L9-VL4-L10-CH1, VL3-L6-VH4-L7-VH3-L8-CH1-L9-VL4-L10-CL, VH3-L6-VL3-L7-VL4-L8-CL-L9-VH4-L10-CH1, VH3-L6-VL3-L7-VL4-L8-CH1-L9-VH4-L10-CL, VH3-L6-VL3-L7-VH4-L8-CL-L9-VL4-L10-CH1, VH3-L6-VL3-L7-VH4-L8-CH1-L9-VL4-L10-CL, VH3-L6-VL4-L7-VL3-L8-CL-L9-VH4-L10-CH1, VH3-L6-VL4-L7-VL3-L8-CH1-L9-VH4-L10-CL, VH3-L6-VH4-L7-VL3-L8-CL-L9-VL4-L10-CH1, VH3-L6-VH4-L7-VL3-L8-CH1-L9-VL4-L10-CL, VL4-L6-VL3-L7-VH4-L8-CL-L9-VH3-L10-CH1, VL4-L6-VL3-L7-VH4-L8-CH1-L9-VH3-L10-CL, VL4-L6-VH3-L7-VH4-L8-CL-L9-VL3-L10-CH1, VL4-L6-VH3-L7-VH4-L8-CH1-L9-VL3-L10-CL, VL4-L6-VH4-L7-VL3-L8-CL-L9-VH3-L10-CH1, VL4-L6-VH4-L7-VL3-L8-CH1-L9-VH3-L10-CL, VL4-L6-VH4-L7-VH3-L8-CL- L9-VL3-L10-CH1, VL4-L6-VH4-L7-VH3-L8-CH1-L9-VL3-L10-CL, VH4-L6-VL3-L7-VL4-L8-CL-L9-VH3-L10-CH1, VH4-L6-VL3-L7-VL4-L8-CH1-L9-VH3-L10-CL, VH4-L6-VH3-L7-VL4-L8-CL-L9-VL3-L10-CH1, VH4-L6-VH3-L7-VL4-L8-CH1-L9-VL3-L10-CL, VH4-L6-VL4-L7-VL3-L8-CL-L9-VH3-L10-CH1, VH4-L6-VL4-L7-VL3-L8-CH1-L9-VH3-L10-CL, VH4-L6-VL4-L7-VH3-L8-CL-L9-VL3-L10-CH1, or VH4-L6-VL4-L7-VH3-L8-CH1-L9-VL3-L10-CL.

In some aspects, an antigen binding polypeptide complex provided herein comprises a first polypeptide, and a second polypeptide; wherein the first polypeptide has a structure represented by VL1-VH1-VL2-CL-VH2-CH1-CH2-CH3, VL1-VH1-VL2-CH1-VH2-CL-CH2-CH3, VL1-VH1-VH2-CL-VL2-CH1-CH2-CH3, VL1-VH1-VH2-CH1-VL2-CL-CH2-CH3, VL1-VL2-VH1-CL-VH2-CH1-CH2-CH3, VL1-VL2-VH1-CH1-VH2-CL-CH2-CH3, VL1-VH2-VH1-CL-VL2-CH1-CH2-CH3, VL1-VH2-VH1-CH1-VL2-CL-CH2-CH3, VH1-VL1-VL2-CL-VH2-CH1-CH2-CH3, VH1-VL1-VL2-CH1-VH2-CL-CH2-CH3, VH1-VL1-VH2-CL-VL2-CH1-CH2-CH3, VH1-VL1-VH2-CH1-VL2-CL-CH2-CH3, VH1-VL2-VL1-CL-VH2-CH1-CH2-CH3, VH1-VL2-VL1-CH1-VH2-CL-CH2-CH3, VH1-VH2-VL1-CL-VL2-CH1-CH2-CH3, VH1-VH2-VL1-CH1-VL2-CL-CH2-CH3, VL2-VL1-VH2-CL-VH1-CH1-CH2-CH3, VL2-VL1-VH2-CH1-VH1-CL-CH2-CH3, VL2-VH1-VH2-CL-VL1-CH1-CH2-CH3, VL2-VH1-VH2-CH1-VL1-CL-CH2-CH3, VL2-VH2-VL1-CL-VH1-CH1-CH2-CH3, VL2-VH2-VL1-CH1-VH1-CL-CH2-CH3, VL2-VH2-VH1-CL-VL1-CH1-CH2-CH3, VL2-VH2-VH1-CH1-VL1-CL-CH2-CH3, VH2-VL1-VL2-CL-VH1-CH1-CH2-CH3, VH2-VL1-VL2-CH1-VH1-CL-CH2-CH3, VH2-VH1-VL2-CL-VL1-CH1-CH2-CH3, VH2-VH1-VL2-CH1-VL1-CL-CH2-CH3, VH2-VL2-VL1-CL-VH1-CH1-CH2-CH3, VH2-VL2-VL1-CH1-VH1-CL-CH2-CH3, VH2-VL2-VH1-CL-VL1-CH1-CH2-CH3, or VH2-VL2-VH1-CH1-VL1-CL-CH2-CH3; and wherein the second polypeptide has a structure represented by VL3-VH3-VL4-CL-VH4-CH1-CH2-CH3, VL3-VH3-VL4-CH1-VH4-CL-CH2-CH3, VL3-VH3-VH4-CL-VL4-CH1-CH2-CH3, VL3-VH3-VH4-CH1-VL4-CL-CH2-CH3, VL3-VL4-VH3-CL-VH4-CH1-CH2-CH3, VL3-VL4-VH3-CH1-VH4-CL-CH2-CH3, VL3-VH4-VH3-CL-VL4-CH1-CH2-CH3, VL3-VH4-VH3-CH1-VL4-CL-CH2-CH3, VH3-VL3-VL4-CL-VH4-CH1-CH2-CH3, VH3-VL3-VL4-CH1-VH4-CL-CH2-CH3, VH3-VL3-VH4-CL-VL4-CH1-CH2-CH3, VH3-VL3-VH4-CH1-VL4-CL-CH2-CH3, VH3-VL4-VL3-CL-VH4-CH1-CH2-CH3, VH3-VL4-VL3-CH1-VH4-CL-CH2-CH3, VH3-VH4-VL3-CL-VL4-CH1-CH2-CH3, VH3-VH4-VL3-CH1-VL4-CL-CH2-CH3, VL4-VL3-VH4-CL-VH3-CH1-CH2-CH3, VL4-VL3-VH4-CH1-VH3-CL-CH2-CH3, VL4-VH3-VH4-CL-VL3-CH1-CH2-CH3, VL4-VH3-VH4-CH1-VL3-CL-CH2-CH3, VL4-VH4-VL3-CL-VH3-CH1-CH2-CH3, VL4-VH4-VL3-CH1-VH3-CL-CH2-CH3, VL4-VH4-VH3-CL-VL3-CH1-CH2-CH3, VL4-VH4-VH3-CH1-VL3-CL-CH2-CH3, VH4-VL3-VL4-CL-VH3-CH1-CH2-CH3, VH4-VL3-VL4-CH1-VH3-CL-CH2-CH3, VH4-VH3-VL4-CL-VL3-CH1-CH2-CH3, VH4-VH3-VL4-CH1-VL3-CL-CH2-CH3, VH4-VL4-VL3-CL-VH3-CH1-CH2-CH3, VH4-VL4-VL3-CH1-VH3-CL-CH2-CH3, VH4-VL4-VH3-CL-VL3-CH1-CH2-CH3, or VH4-VL4-VH3-CH1-VL3-CL-CH2-CH3.

In some aspects, an antigen binding polypeptide complex provided herein comprises a first polypeptide, and a second polypeptide; wherein the first polypeptide has a structure represented by VL1-L1-VH1-L2-VL2-L3-CL-L4-VH2-L5-CH1-CH2-CH3, VL1-L1-VH1-L2-VL2-L3-CH1-L4-VH2- L5-CL-CH2-CH3, VL1-L1-VH1-L2-VH2-L3-CL-L4-VL2-L5-CH1-CH2-CH3, VL1-L1-VH1-L2-VH2-L3-CH1-L4-VL2-L5-CL-CH2-CH3, VL1-L1-VL2-L2-VH1-L3-CL-L4-VH2-L5-CH1-CH2-CH3, VL1-L1-VL2-L2-VH1-L3-CH1-L4-VH2-L5-CL-CH2-CH3, VL1-L1-VH2-L2-VH1-L3-CL-L4-VL2-L5-CH1-CH2-CH3, VL1-L1-VH2-L2-VH1-L3-CH1-L4-VL2-L5-CL-CH2-CH3, VH1-L1-VL1-L2-VL2-L3-CL-L4-VH2-L5-CH1-CH2-CH3, VH1-L1-VL1-L2-VL2-L3-CH1-L4-VH2-L5-CL-CH2-CH3, VH1-L1-VL1-L2-VH2-L3-CL-L4-VL2-L5-CH1-CH2-CH3, VH1-L1-VL1-L2-VH2-L3-CH1-L4-VL2-L5-CL-CH2-CH3, VH1-L1-VL2-L2-VL1-L3-CL-L4-VH2-L5-CH1-CH2-CH3, VH1-L1-VL2-L2-VL1-L3-CH1-L4-VH2-L5- CL-CH2-CH3, VH1-L1-VH2-L2-VL1-L3-CL-L4-VL2-L5-CH1-CH2-CH3, VH1-L1-VH2-L2-VL1-L3-CH1-L4-VL2-L5-CL-CH2-CH3, VL2-L1-VL1-L2-VH2-L3-CL-L4-VH1-L5-CH1-CH2-CH3, VL2-L1-VL1-L2-VH2-L3-CH1-L4-VH1-L5-CL-CH2-CH3, VL2-L1-VH1-L2-VH2-L3-CL-L4-VL1-L5-CH1-CH2-CH3, VL2-L1-VH1-L2-VH2-L3-CH1-L4-VL1-L5-CL-CH2-CH3, VL2-L1-VH2-L2-VL1-L3-CL-L4-VH1-L5-CH1-CH2-CH3, VL2-L1-VH2-L2-VL1-L3-CH1-L4-VH1-L5-CL-CH2-CH3, VL2-L1-VH2-L2-VH1-L3-CL-L4-VL1-L5-CH1-CH2-CH3, VL2-L1-VH2-L2-VH1-L3-CH1-L4-VL1-L5-CL-CH2-CH3, VH2-L1-VL1-L2-VL2-L3-CL-L4-VH1-L5-CH1-CH2-CH3, VH2-L1-VL1-L2-VL2-L3-CH1-L4-VH1-L5- CL-CH2-CH3, VH2-L1-VH1-L2-VL2-L3-CL-L4-VL1-L5-CH1-CH2-CH3, VH2-L1-VH1-L2-VL2-L3-CH1-L4-VL1-L5-CL-CH2-CH3, VH2-L1-VL2-L2-VL1-L3-CL-L4-VH1-L5-CH1-CH2-CH3, VH2-L1-VL2-L2-VL1-L3-CH1-L4-VH1-L5-CL-CH2-CH3, VH2-L1-VL2-L2-VH1-L3-CL-L4-VL1-L5-CH1-CH2-CH3, or VH2-L1-VL2-L2-VH1-L3-CH1-L4-VL1-L5-CL-CH2-CH3; and wherein the second polypeptide has a structure represented by VL3-L6-VH3-L7-VL4-L8-CL-L9-VH4-L10-CH1-CH2-CH3, VL3-L6-VH3-L7-VL4-L8-CH1-L9-VH4-L10-CL-CH2-CH3, VL3-L6-VH3-L7-VH4-L8-CL-L9-VL4-L10-CH1-CH2-CH3, VL3-L6-VH3-L7-VH4-L8-CH1-L9-VL4-L10-CL-CH2-CH3, VL3-L6-VL4-L7-VH3-L8-CL-L9-VH4-L10-CH1-CH2-CH3, VL3-L6-VL4-L7-VH3-L8-CH1-L9-VH4-L10-CL-CH2-CH3, VL3-L6-VH4-L7-VH3-L8-CL-L9-VL4-L10-CH1-CH2-CH3, VL3-L6-VH4-L7-VH3-L8-CH1-L9-VL4-L10-CL-CH2-CH3, VH3- L6-VL3-L7-VL4-L8-CL-L9-VH4-L10-CH1-CH2-CH3, VH3-L6-VL3-L7-VL4-L8-CH1-L9-VH4-L10-CL-CH2-CH3, VH3-L6-VL3-L7-VH4-L8-CL-L9-VL4-L10-CH1-CH2-CH3, VH3-L6-VL3-L7-VH4-L8- CH1-L9-VL4-L10-CL-CH2-CH3, VH3-L6-VL4-L7-VL3-L8-CL-L9-VH4-L10-CH1-CH2-CH3, VH3-L6- VL4-L7-VL3-L8-CH1-L9-VH4-L10-CL-CH2-CH3, VH3-L6-VH4-L7-VL3-L8-CL-L9-VL4-L10-CH1-CH2-CH3, VH3-L6-VH4-L7-VL3-L8-CH1-L9-VL4-L10-CL-CH2-CH3, VL4-L6-VL3-L7-VH4-L8-CL-L9-VH3-L10-CH1-CH2-CH3, VL4-L6-VL3-L7-VH4-L8-CH1-L9-VH3-L10-CL-CH2-CH3, VL4-L6-VH3-L7-VH4-L8-CL-L9-VL3-L10-CH1-CH2-CH3, VL4-L6-VH3-L7-VH4-L8-CH1-L9-VL3-L10-CL-CH2-CH3, VL4-L6-VH4-L7-VL3-L8-CL-L9-VH3-L10-CH1-CH2-CH3, VL4-L6-VH4-L7-VL3-L8-CH1-L9-VH3-L10-CL-CH2-CH3, VL4-L6-VH4-L7-VH3-L8-CL-L9-VL3-L10-CH1-CH2-CH3, VL4-L6-VH4- L7-VH3-L8-CH1-L9-VL3-L10-CL-CH2-CH3, VH4-L6-VL3-L7-VL4-L8-CL-L9-VH3-L10-CH1-CH2-CH3, VH4-L6-VL3-L7-VL4-L8-CH1-L9-VH3-L10-CL-CH2-CH3, VH4-L6-VH3-L7-VL4-L8-CL-L9-VL3-L10-CH1-CH2-CH3, VH4-L6-VH3-L7-VL4-L8-CH1-L9-VL3-L10-CL-CH2-CH3, VH4-L6-VL4-L7-VL3-L8-CL-L9-VH3-L10-CH1-CH2-CH3, VH4-L6-VL4-L7-VL3-L8-CH1-L9-VH3-L10-CL-CH2-CH3, VH4- L6-VL4-L7-VH3-L8-CL-L9-VL3-L10-CH1-CH2-CH3, or VH4-L6-VL4-L7-VH3-L8-CH1-L9-VL3-L10-CL-CH2-CH3.

In some aspects, an antigen binding polypeptide complex provided herein comprises a first polypeptide, and a second polypeptide; wherein the first polypeptide has a structure represented by VL1-CL-VH1-CH1-VL2-VH2, VL1-CL-VH1-CH1-VH2-VL2, VL1-CL-VL2-CH1-VH1-VH2, VL1-CL-VH2-CH1-VH1-VL2, VL1-CH1-VH1-CL-VL2-VH2, VL1-CH1-VH1-CL-VH2-VL2, VL1-CH1-VL2-CL-VH1-VH2, VL1-CH1-VH2-CL-VH1-VL2, VH1-CL-VL1-CH1-VL2-VH2, VH1-CL-VL1-CH1-VH2-VL2, VH1-CL-VL2-CH1-VL1-VH2, VH1-CL-VH2-CH1-VL1-VL2, VH1-CH1-VL1-CL-VL2-VH2, VH1-CH1-VL1-CL-VH2-VL2, VH1-CH1-VL2-CL-VL1-VH2, VH1-CH1-VH2-CL-VL1-VL2, VL2-CL-VL1-CH1-VH2-VH1, VL2-CL-VH1-CH1-VH2-VL1, VL2-CL-VH2-CH1-VL1-VH1, VL2-CL-VH2-CH1-VH1-VL1, VL2-CH1-VL1-CL-VH2-VH1, VL2-CH1-VH1-CL-VH2-VL1, VL2-CH1-VH2-CL-VL1-VH1, VL2-CH1-VH2-CL-VH1-VL1, VH2-CL-VL1-CH1-VL2-VH1, VH2-CL-VH1-CH1-VL2-VL1, VH2-CL-VL2-CH1-VL1-VH1, VH2-CL-VL2-CH1-VH1-VL1, VH2-CH1-VL1-CL-VL2-VH1, VH2-CH1-VH1-CL-VL2-VL1, VH2-CH1-VL2-CL-VL1-VH1, or VH2-CH1-VL2-CL-VH1-VL1; and wherein the second polypeptide has a structure represented by VL3-CL-VH3-CH1-VL4-VH4, VL3-CL-VH3-CH1-VH4-VL4, VL3-CL-VL4-CH1-VH3-VH4, VL3-CL-VH4-CH1-VH3-VL4, VL3-CH1-VH3-CL-VL4-VH4, VL3-CH1-VH3-CL-VH4-VL4, VL3-CH1-VL4-CL-VH3-VH4, VL3-CH1-VH4-CL-VH3-VL4, VH3-CL-VL3-CH1-VL4-VH4, VH3-CL-VL3-CH1-VH4-VL4, VH3-CL-VL4-CH1-VL3-VH4, VH3-CL-VH4-CH1-VL3-VL4, VH3-CH1-VL3-CL-VL4-VH4, VH3-CH1-VL3-CL-VH4-VL4, VH3-CH1-VL4-CL-VL3-VH4, VH3-CH1-VH4-CL-VL3-VL4, VL4-CL-VL3-CH1-VH4-VH3, VL4-CL-VH3-CH1-VH4-VL3, VL4-CL-VH4-CH1-VL3-VH3, VL4-CL-VH4-CH1-VH3-VL3, VL4-CH1-VL3-CL-VH4-VH3, VL4-CH1-VH3-CL-VH4-VL3, VL4-CH1-VH4-CL-VL3-VH3, VL4-CH1-VH4-CL-VH3-VL3, VH4-CL-VL3-CH1-VL4-VH3, VH4-CL-VH3-CH1-VL4-VL3, VH4-CL-VL4-CH1-VL3-VH3, VH4-CL-VL4-CH1-VH3-VL3, VH4-CH1-VL3-CL-VL4-VH3, VH4-CH1-VH3-CL-VL4-VL3, VH4-CH1-VL4-CL-VL3-VH3, or VH4-CH1-VL4-CL-VH3-VL3.

In some aspects, an antigen binding polypeptide complex provided herein comprises a first polypeptide, and a second polypeptide; wherein the first polypeptide has a structure represented by VL1-L1-CL-L2-VH1-L3-CH1-L4-VL2-L5-VH2, VL1-L1-CL-L2-VH1-L3-CH1-L4-VH2-L5-VL2, VL1-L1-CL-L2-VL2-L3-CH1-L4-VH1-L5-VH2, VL1-L1-CL-L2-VH2-L3-CH1-L4-VH1-L5-VL2, VL1-L1-CH1-L2-VH1-L3-CL-L4-VL2-L5-VH2, VL1-L1-CH1-L2-VH1-L3-CL-L4-VH2-L5-VL2, VL1-L1-CH1-L2-VL2-L3-CL-L4-VH1-L5-VH2, VL1-L1-CH1-L2-VH2-L3-CL-L4-VH1-L5-VL2, VH1-L1-CL-L2-VL1-L3-CH1-L4-VL2-L5-VH2, VH1-L1-CL-L2-VL1-L3-CH1-L4-VH2-L5-VL2, VH1-L1-CL-L2-VL2-L3-CH1-L4-VL1-L5-VH2, VH1-L1-CL-L2-VH2-L3-CH1-L4-VL1-L5-VL2, VH1-L1-CH1-L2-VL1-L3- CL-L4-VL2-L5-VH2, VH1-L1-CH1-L2-VL1-L3-CL-L4-VH2-L5-VL2, VH1-L1-CH1-L2-VL2-L3-CL-L4-VL1-L5-VH2, VH1-L1-CH1-L2-VH2-L3-CL-L4-VL1-L5-VL2, VL2-L1-CL-L2-VL1-L3-CH1-L4-VH2-L5-VH1, VL2-L1-CL-L2-VH1-L3-CH1-L4-VH2-L5-VL1, VL2-L1-CL-L2-VH2-L3-CH1-L4-VL1-L5-VH1, VL2-L1-CL-L2-VH2-L3-CH1-L4-VH1-L5-VL1, VL2-L1-CH1-L2-VL1-L3-CL-L4-VH2-L5-VH1, VL2-L1-CH1-L2-VH1-L3-CL-L4-VH2-L5-VL1, VL2-L1-CH1-L2-VH2-L3-CL-L4-VL1-L5-VH1, VL2-L1-CH1-L2-VH2-L3-CL-L4-VH1-L5-VL1, VH2-L1-CL-L2-VL1-L3-CH1-L4-VL2-L5-VH1, VH2-L1-CL-L2-VH1-L3-CH1-L4-VL2-L5-VL1, VH2-L1-CL-L2-VL2-L3-CH1-L4-VL1-L5-VH1, VH2-L1-CL- L2-VL2-L3-CH1-L4-VH1-L5-VL1, VH2-L1-CH1-L2-VL1-L3-CL-L4-VL2-L5-VH1, VH2-L1-CH1-L2-VH1-L3-CL-L4-VL2-L5-VL1, VH2-L1-CH1-L2-VL2-L3-CL-L4-VL1-L5-VH1, or VH2-L1-CH1-L2-VL2-L3-CL-L4-VH1-L5-VL1; and wherein the second polypeptide has a structure represented by VL3-L6-CL-L7-VH3-L8-CH1-L9-VL4-L10-VH4, VL3-L6-CL-L7-VH3-L8-CH1-L9-VH4-L10-VL4, VL3-L6-CL-L7-VL4-L8-CH1-L9-VH3-L10-VH4, VL3-L6-CL-L7-VH4-L8-CH1-L9-VH3-L10-VL4, VL3-L6-CH1-L7-VH3-L8-CL-L9-VL4-L10-VH4, VL3-L6-CH1-L7-VH3-L8-CL-L9-VH4-L10-VL4, VL3-L6-CH1-L7-VL4-L8-CL-L9-VH3-L10-VH4, VL3-L6-CH1-L7-VH4-L8-CL-L9-VH3-L10-VL4, VH3-L6-CL-L7- VL3-L8-CH1-L9-VL4-L10-VH4, VH3-L6-CL-L7-VL3-L8-CH1-L9-VH4-L10-VL4, VH3-L6-CL-L7-VL4-L8-CH1-L9-VL3-L10-VH4, VH3-L6-CL-L7-VH4-L8-CH1-L9-VL3-L10-VL4, VH3-L6-CH1-L7-VL3-L8-CL-L9-VL4-L10-VH4, VH3-L6-CH1-L7-VL3-L8-CL-L9-VH4-L10-VL4, VH3-L6-CH1-L7-VL4-L8-CL-L9-VL3-L10-VH4, VH3-L6-CH1-L7-VH4-L8-CL-L9-VL3-L10-VL4, VL4-L6-CL-L7-VL3-L8-CH1- L9-VH4-L10-VH3, VL4-L6-CL-L7-VH3-L8-CH1-L9-VH4-L10-VL3, VL4-L6-CL-L7-VH4-L8-CH1-L9-VL3-L10-VH3, VL4-L6-CL-L7-VH4-L8-CH1-L9-VH3-L10-VL3, VL4-L6-CH1-L7-VL3-L8-CL-L9-VH4-L10-VH3, VL4-L6-CH1-L7-VH3-L8-CL-L9-VH4-L10-VL3, VL4-L6-CH1-L7-VH4-L8-CL-L9-VL3-L10-VH3, VL4-L6-CH1-L7-VH4-L8-CL-L9-VH3-L10-VL3, VH4-L6-CL-L7-VL3-L8-CH1-L9-VL4-L10-VH3, VH4-L6-CL-L7-VH3-L8-CH1-L9-VL4-L10-VL3, VH4-L6-CL-L7-VL4-L8-CH1-L9-VL3-L10-VH3, VH4-L6-CL-L7-VL4-L8-CH1-L9-VH3-L10-VL3, VH4-L6-CH1-L7-VL3-L8-CL-L9-VL4-L10-VH3, VH4-L6-CH1-L7-VH3-L8-CL-L9-VL4-L10-VL3, VH4-L6-CH1-L7-VL4-L8-CL-L9-VL3-L10-VH3, or VH4-L6-CH1-L7-VL4-L8-CL-L9-VH3-L10-VL3.

In some aspects, an antigen binding polypeptide complex provided herein comprises a first polypeptide, and a second polypeptide; wherein the first polypeptide has a structure represented by VL1-CL-VH1-CH1-VL2-VH2-CH2-CH3, VL1-CL-VH1-CH1-VH2-VL2-CH2-CH3, VL1-CL-VL2-CH1-VH1-VH2-CH2-CH3, VL1-CL-VH2-CH1-VH1-VL2-CH2-CH3, VL1-CH1-VH1-CL-VL2-VH2-CH2-CH3, VL1-CH1-VH1-CL-VH2-VL2-CH2-CH3, VL1-CH1-VL2-CL-VH1-VH2-CH2-CH3, VL1-CH1-VH2-CL-VH1-VL2-CH2-CH3, VH1-CL-VL1-CH1-VL2-VH2-CH2-CH3, VH1-CL-VL1-CH1-VH2-VL2-CH2-CH3, VH1-CL-VL2-CH1-VL1-VH2-CH2-CH3, VH1-CL-VH2-CH1-VL1-VL2-CH2-CH3, VH1-CH1-VL1-CL-VL2-VH2-CH2-CH3, VH1-CH1-VL1-CL-VH2-VL2-CH2-CH3, VH1-CH1-VL2-CL-VL1-VH2-CH2-CH3, VH1-CH1-VH2-CL-VL1-VL2-CH2-CH3, VL2-CL-VL1-CH1-VH2-VH1-CH2-CH3, VL2-CL-VH1-CH1-VH2-VL1-CH2-CH3, VL2-CL-VH2-CH1-VL1-VH1-CH2-CH3, VL2-CL-VH2-CH1-VH1-VL1-CH2-CH3, VL2-CH1-VL1-CL-VH2-VH1-CH2-CH3, VL2-CH1-VH1-CL-VH2-VL1-CH2-CH3, VL2-CH1-VH2-CL-VL1-VH1-CH2-CH3, VL2-CH1-VH2-CL-VH1-VL1-CH2-CH3, VH2-CL-VL1-CH1-VL2-VH1-CH2-CH3, VH2-CL-VH1-CH1-VL2-VL1-CH2-CH3, VH2-CL-VL2-CH1-VL1-VH1-CH2-CH3, VH2-CL-VL2-CH1-VH1-VL1-CH2-CH3, VH2-CH1-VL1-CL-VL2-VH1-CH2-CH3, VH2-CH1-VH1-CL-VL2-VL1-CH2-CH3, VH2-CH1-VL2-CL-VL1-VH1-CH2-CH3, or VH2-CH1-VL2-CL-VH1-VL1-CH2-CH3; and wherein the second polypeptide has a structure represented by VL3-CL-VH3-CH1-VL4-VH4-CH2-CH3, VL3-CL-VH3-CH1-VH4-VL4-CH2-CH3, VL3-CL-VL4-CH1-VH3-VH4-CH2-CH3, VL3-CL-VH4-CH1-VH3-VL4-CH2-CH3, VL3-CH1-VH3-CL-VL4-VH4-CH2-CH3, VL3-CH1-VH3-CL-VH4-VL4-CH2-CH3, VL3-CH1-VL4-CL-VH3-VH4-CH2-CH3, VL3-CH1-VH4-CL-VH3-VL4-CH2-CH3, VH3-CL-VL3-CH1-VL4-VH4-CH2-CH3, VH3-CL-VL3-CH1-VH4-VL4-CH2-CH3, VH3-CL-VL4-CH1-VL3-VH4-CH2-CH3, VH3-CL-VH4-CH1-VL3-VL4-CH2-CH3, VH3-CH1-VL3-CL-VL4-VH4-CH2-CH3, VH3-CH1-VL3-CL-VH4-VL4-CH2-CH3, VH3-CH1-VL4-CL-VL3-VH4-CH2-CH3, VH3-CH1-VH4-CL-VL3-VL4-CH2-CH3, VL4-CL-VL3-CH1-VH4-VH3-CH2-CH3, VL4-CL-VH3-CH1-VH4-VL3-CH2-CH3, VL4-CL-VH4-CH1-VL3-VH3-CH2-CH3, VL4-CL-VH4-CH1-VH3-VL3-CH2-CH3, VL4-CH1-VL3-CL-VH4-VH3-CH2-CH3, VL4-CH1-VH3-CL-VH4-VL3-CH2-CH3, VL4-CH1-VH4-CL-VL3-VH3-CH2-CH3, VL4-CH1-VH4-CL-VH3-VL3-CH2-CH3, VH4-CL-VL3-CH1-VL4-VH3-CH2-CH3, VH4-CL-VH3-CH1-VL4-VL3-CH2-CH3, VH4-CL-VL4-CH1-VL3-VH3-CH2-CH3, VH4-CL-VL4-CH1-VH3-VL3-CH2-CH3, VH4-CH1-VL3-CL-VL4-VH3-CH2-CH3, VH4-CH1-VH3-CL-VL4-VL3-CH2-CH3, VH4-CH1-VL4-CL-VL3-VH3-CH2-CH3, or VH4-CH1-VL4-CL-VH3-VL3-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-L4-VL2-L5-VH2-CH2-CH3, VL1-L1-CL-L2-VH1-L3-CH1-L4-VH2-L5-VL2-CH2-CH3, VL1-L1-CL-L2-VL2-L3-CH1-L4-VH1-L5-VH2-CH2-CH3, VL1-L1-CL-L2-VH2-L3-CH1-L4-VH1-L5-VL2-CH2-CH3, VL1-L1-CH1-L2-VH1-L3-CL-L4-VL2-L5-VH2-CH2-CH3, VL1-L1-CH1-L2-VH1-L3-CL-L4-VH2-L5-VL2-CH2-CH3, VL1-L1-CH1-L2-VL2-L3-CL-L4-VH1-L5-VH2-CH2-CH3, VL1-L1-CH1-L2-VH2-L3-CL-L4-VH1-L5-VL2-CH2-CH3, VH1-L1-CL-L2-VL1-L3-CH1-L4-VL2-L5-VH2-CH2-CH3, VH1-L1-CL-L2-VL1-L3-CH1-L4-VH2-L5-VL2-CH2-CH3, VH1-L1-CL-L2-VL2-L3-CH1-L4-VL1-L5-VH2-CH2-CH3, VH1-L1-CL-L2-VH2-L3-CH1-L4-VL1-L5-VL2-CH2-CH3, VH1-L1-CH1-L2-VL1-L3-CL-L4-VL2-L5-VH2-CH2-CH3, VH1-L1-CH1-L2-VL1-L3-CL-L4-VH2-L5-VL2-CH2-CH3, VH1-L1-CH1-L2-VL2-L3-CL-L4-VL1-L5-VH2-CH2-CH3, VH1-L1-CH1-L2-VH2-L3-CL-L4-VL1-L5-VL2-CH2-CH3, VL2-L1-CL-L2-VL1-L3-CH1-L4-VH2-L5-VH1-CH2-CH3, VL2-L1-CL-L2-VH1-L3-CH1-L4-VH2-L5-VL1-CH2-CH3, VL2-L1-CL-L2-VH2-L3-CH1-L4-VL1-L5-VH1-CH2-CH3, VL2-L1-CL-L2-VH2-L3-CH1-L4-VH1-L5-VL1-CH2-CH3, VL2-L1-CH1-L2-VL1-L3-CL-L4-VH2-L5-VH1-CH2-CH3, VL2-L1-CH1-L2-VH1-L3-CL-L4-VH2-L5-VL1-CH2-CH3, VL2-L1-CH1-L2-VH2-L3-CL-L4-VL1-L5-VH1-CH2-CH3, VL2-L1-CH1-L2-VH2-L3-CL-L4-VH1-L5-VL1-CH2-CH3, VH2-L1-CL-L2-VL1-L3-CH1-L4-VL2-L5-VH1-CH2-CH3, VH2-L1-CL-L2-VH1-L3-CH1-L4-VL2-L5-VL1-CH2-CH3, VH2-L1-CL-L2-VL2-L3-CH1-L4-VL1-L5-VH1-CH2-CH3, VH2-L1-CL-L2-VL2-L3-CH1-L4-VH1-L5-VL1-CH2-CH3, VH2-L1-CH1-L2-VL1-L3-CL-L4-VL2-L5-VH1-CH2-CH3, VH2-L1-CH1-L2-VH1-L3-CL-L4-VL2-L5-VL1-CH2-CH3, VH2-L1-CH1-L2-VL2-L3-CL-L4-VL1-L5-VH1-CH2-CH3, or VH2-L1-CH1-L2-VL2-L3-CL-L4-VH1-L5-VL1-CH2-CH3; and wherein the second polypeptide has a structure represented by VL3-L6-CL-L7-VH3-L8-CH1-L9-VL4-L10-VH4-CH2-CH3, VL3-L6-CL-L7-VH3-L8-CH1-L9-VH4-L10-VL4-CH2-CH3, VL3-L6-CL-L7-VL4-L8-CH1-L9-VH3-L10-VH4-CH2-CH3, VL3-L6-CL-L7- VH4-L8-CH1-L9-VH3-L10-VL4-CH2-CH3, VL3-L6-CH1-L7-VH3-L8-CL-L9-VL4-L10-VH4-CH2-CH3, VL3-L6-CH1-L7-VH3-L8-CL-L9-VH4-L10-VL4-CH2-CH3, VL3-L6-CH1-L7-VL4-L8-CL-L9-VH3-L10-VH4-CH2-CH3, VL3-L6-CH1-L7-VH4-L8-CL-L9-VH3-L10-VL4-CH2-CH3, VH3-L6-CL-L7-VL3-L8-CH1-L9-VL4-L10-VH4-CH2-CH3, VH3-L6-CL-L7-VL3-L8-CH1-L9-VH4-L10-VL4-CH2-CH3, VH3-L6-CL-L7-VL4-L8-CH1-L9-VL3-L10-VH4-CH2-CH3, VH3-L6-CL-L7-VH4-L8-CH1-L9-VL3-L10-VL4- CH2-CH3, VH3-L6-CH1-L7-VL3-L8-CL-L9-VL4-L10-VH4-CH2-CH3, VH3-L6-CH1-L7-VL3-L8-CL-L9-VH4-L10-VL4-CH2-CH3, VH3-L6-CH1-L7-VL4-L8-CL-L9-VL3-L10-VH4-CH2-CH3, VH3-L6-CH1-L7-VH4-L8-CL-L9-VL3-L10-VL4-CH2-CH3, VL4-L6-CL-L7-VL3-L8-CH1-L9-VH4-L10-VH3-CH2-CH3, VL4-L6-CL-L7-VH3-L8-CH1-L9-VH4-L10-VL3-CH2-CH3, VL4-L6-CL-L7-VH4-L8-CH1-L9-VL3-L10-VH3-CH2-CH3, VL4-L6-CL-L7-VH4-L8-CH1-L9-VH3-L10-VL3-CH2-CH3, VL4-L6-CH1-L7-VL3-L8-CL-L9-VH4-L10-VH3-CH2-CH3, VL4-L6-CH1-L7-VH3-L8-CL-L9-VH4-L10-VL3-CH2-CH3, VL4-L6-CH1-L7-VH4-L8-CL-L9-VL3-L10-VH3-CH2-CH3, VL4-L6-CH1-L7-VH4-L8-CL-L9-VH3-L10-VL3-CH2-CH3, VH4-L6-CL-L7-VL3-L8-CH1-L9-VL4-L10-VH3-CH2-CH3, VH4-L6-CL-L7-VH3-L8-CH1-L9-VL4-L10-VL3-CH2-CH3, VH4-L6-CL-L7-VL4-L8-CH1-L9-VL3-L10-VH3-CH2-CH3, VH4-L6-CL-L7-VL4-L8-CH1-L9-VH3-L10-VL3-CH2-CH3, VH4-L6-CH1-L7-VL3-L8-CL-L9-VL4-L10-VH3-CH2-CH3, VH4-L6-CH1-L7-VH3-L8-CL-L9-VL4-L10-VL3-CH2-CH3, VH4-L6-CH1-L7-VL4-L8-CL-L9-VL3-L10-VH3-CH2-CH3, or VH4-L6-CH1-L7-VL4-L8-CL-L9-VH3-L10-VL3-CH2-CH3.

In some aspects, an antigen binding polypeptide complex provided herein comprises a first polypeptide, and a second polypeptide; wherein the first polypeptide has a structure represented by VL1-VL2-VH2-VH1-CL-CH1, VL1-VL2-VH2-VH1-CH1-CL, VL1-VH2-VL2-VH1-CL-CH1, VL1-VH2-VL2-VH1-CH1-CL, VH1-VL2-VH2-VL1-CL-CH1, VH1-VL2-VH2-VL1-CH1-CL, VH1-VH2-VL2-VL1-CL-CH1, VH1-VH2-VL2-VL1-CH1-CL, VL2-VL1-VH1-VH2-CL-CH1, VL2-VL1-VH1-VH2-CH1-CL, VL2-VH1-VL1-VH2-CL-CH1, VL2-VH1-VL1-VH2-CH1-CL, VH2-VL1-VH1-VL2-CL-CH1, VH2-VL1-VH1-VL2-CH1-CL, VH2-VH1-VL1-VL2-CL-CH1, or VH2-VH1-VL1-VL2-CH1-CL; and wherein the second polypeptide has a structure represented by VL3-VL4-VH4-VH3-CL-CH1, VL3-VL4-VH4-VH3-CH1-CL, VL3-VH4-VL4-VH3-CL-CH1, VL3-VH4-VL4-VH3-CH1-CL, VH3-VL4-VH4-VL3-CL-CH1, VH3-VL4-VH4-VL3-CH1-CL, VH3-VH4-VL4-VL3-CL-CH1, VH3-VH4-VL4-VL3-CH1-CL, VL4-VL3-VH3-VH4-CL-CH1, VL4-VL3-VH3-VH4-CH1-CL, VL4-VH3-VL3-VH4-CL-CH1, VL4-VH3-VL3-VH4-CH1-CL, VH4-VL3-VH3-VL4-CL-CH1, VH4-VL3-VH3-VL4-CH1-CL, VH4-VH3-VL3-VL4-CL-CH1, or VH4-VH3-VL3-VL4-CH1-CL.

In some aspects, an antigen binding polypeptide complex provided herein comprises a first polypeptide, and a second polypeptide; wherein the first polypeptide has a structure represented by VL1-L1-VL2-L2-VH2-L3-VH1-L4-CL-L5-CH1, VL1-L1-VL2-L2-VH2-L3-VH1-L4-CH1-L5-CL, VL1-L1-VH2-L2-VL2-L3-VH1-L4-CL-L5-CH1, VL1-L1-VH2-L2-VL2-L3-VH1-L4-CH1-L5-CL, VH1-L1-VL2-L2-VH2-L3-VL1-L4-CL-L5-CH1, VH1-L1-VL2-L2-VH2-L3-VL1-L4-CH1-L5-CL, VH1-L1-VH2-L2-VL2-L3-VL1-L4-CL-L5-CH1, VH1-L1-VH2-L2-VL2-L3-VL1-L4-CH1-L5-CL, VL2-L1-VL1-L2-VH1-L3-VH2-L4-CL-L5-CH1, VL2-L1-VL1-L2-VH1-L3-VH2-L4-CH1-L5-CL, VL2-L1-VH1-L2-VL1-L3-VH2-L4-CL-L5-CH1, VL2-L1-VH1-L2-VL1-L3-VH2-L4-CH1-L5-CL, VH2-L1-VL1-L2-VH1-L3-VL2-L4-CL-L5-CH1, VH2-L1-VL1-L2-VH1-L3-VL2-L4-CH1-L5-CL, VH2-L1-VH1-L2-VL1-L3-VL2-L4-CL-L5-CH1, or VH2-L1-VH1-L2-VL1-L3-VL2-L4-CH1-L5-CL; and wherein the second polypeptide has a structure represented by VL3-L6-VL4-L7-VH4-L8-VH3-L9-CL-L10-CH1, VL3-L6-VL4-L7-VH4-L8-VH3-L9-CH1-L10-CL, VL3-L6-VH4-L7-VL4-L8-VH3-L9-CL-L10-CH1, VL3-L6-VH4-L7-VL4-L8-VH3-L9-CH1-L10-CL, VH3-L6-VL4-L7-VH4-L8-VL3-L9-CL-L10-CH1, VH3-L6-VL4-L7-VH4-L8-VL3-L9-CH1-L10-CL, VH3-L6-VH4-L7-VL4-L8-VL3-L9-CL-L10-CH1, VH3-L6-VH4-L7-VL4-L8-VL3-L9-CH1-L10-CL, VL4-L6-VL3-L7-VH3-L8-VH4-L9-CL-L10-CH1, VL4-L6-VL3-L7-VH3-L8-VH4-L9-CH1-L10-CL, VL4-L6-VH3-L7-VL3-L8-VH4-L9-CL-L10-CH1, VL4-L6-VH3-L7-VL3-L8-VH4-L9-CH1-L10-CL, VH4-L6-VL3-L7-VH3-L8-VL4-L9-CL-L10-CH1, VH4-L6-VL3-L7-VH3-L8-VL4-L9-CH1-L10-CL, VH4-L6-VH3-L7-VL3-L8-VL4-L9-CL-L10-CH1, or VH4-L6-VH3-L7-VL3- L8-VL4-L9-CH1-L10-CL.

In some aspects, an antigen binding polypeptide complex provided herein comprises a first polypeptide, and a second polypeptide; wherein the first polypeptide has a structure represented by VL1-VL2-VH2-VH1-CL-CH1-CH2-CH3, VL1-VL2-VH2-VH1-CH1-CL-CH2-CH3, VL1-VH2-VL2-VH1-CL-CH1-CH2-CH3, VL1-VH2-VL2-VH1-CH1-CL-CH2-CH3, VH1-VL2-VH2-VL1-CL-CH1-CH2-CH3, VH1-VL2-VH2-VL1-CH1-CL-CH2-CH3, VH1-VH2-VL2-VL1-CL-CH1-CH2-CH3, VH1-VH2-VL2-VL1-CH1-CL-CH2-CH3, VL2-VL1-VH1-VH2-CL-CH1-CH2-CH3, VL2-VL1-VH1-VH2-CH1-CL-CH2-CH3, VL2-VH1-VL1-VH2-CL-CH1-CH2-CH3, VL2-VH1-VL1-VH2-CH1-CL-CH2-CH3, VH2-VL1-VH1-VL2-CL-CH1-CH2-CH3, VH2-VL1-VH1-VL2-CH1-CL-CH2-CH3, VH2-VH1-VL1-VL2-CL-CH1-CH2-CH3, or VH2-VH1-VL1-VL2-CH1-CL-CH2-CH3; and wherein the second polypeptide has a structure represented by VL3-VL4-VH4-VH3-CL-CH1-CH2-CH3, VL3-VL4-VH4-VH3-CH1-CL-CH2-CH3, VL3-VH4-VL4-VH3-CL-CH1-CH2-CH3, VL3-VH4-VL4-VH3-CH1-CL-CH2-CH3, VH3-VL4-VH4-VL3-CL-CH1-CH2-CH3, VH3-VL4-VH4-VL3-CH1-CL-CH2-CH3, VH3-VH4-VL4-VL3-CL-CH1-CH2-CH3, VH3-VH4-VL4-VL3-CH1-CL-CH2-CH3, VL4-VL3-VH3-VH4-CL-CH1-CH2-CH3, VL4-VL3-VH3-VH4-CH1-CL-CH2-CH3, VL4-VH3-VL3-VH4-CL-CH1-CH2-CH3, VL4-VH3-VL3-VH4-CH1-CL-CH2-CH3, VH4-VL3-VH3-VL4-CL-CH1-CH2-CH3, VH4-VL3-VH3-VL4-CH1-CL-CH2-CH3, VH4-VH3-VL3-VL4-CL-CH1-CH2-CH3, or VH4-VH3-VL3-VL4-CH1-CL-CH2-CH3.

In some aspects, an antigen binding polypeptide complex provided herein comprises a first polypeptide, and a second polypeptide; wherein the first polypeptide has a structure represented by VL1-L1-VL2-L2-VH2-L3-VH1-L4-CL-L5-CH1-CH2-CH3, VL1-L1-VL2-L2-VH2-L3-VH1-L4-CH1- L5-CL-CH2-CH3, VL1-L1-VH2-L2-VL2-L3-VH1-L4-CL-L5-CH1-CH2-CH3, VL1-L1-VH2-L2-VL2-L3-VH1-L4-CH1-L5-CL-CH2-CH3, VH1-L1-VL2-L2-VH2-L3-VL1-L4-CL-L5-CH1-CH2-CH3, VH1-L1-VL2-L2-VH2-L3-VL1-L4-CH1-L5-CL-CH2-CH3, VH1-L1-VH2-L2-VL2-L3-VL1-L4-CL-L5-CH1-CH2-CH3, VH1-L1-VH2-L2-VL2-L3-VL1-L4-CH1-L5-CL-CH2-CH3, VL2-L1-VL1-L2-VH1-L3-VH2-L4- CL-L5-CH1-CH2-CH3, VL2-L1-VL1-L2-VH1-L3-VH2-L4-CH1-L5-CL-CH2-CH3, VL2-L1-VH1-L2-VL1-L3-VH2-L4-CL-L5-CH1-CH2-CH3, VL2-L1-VH1-L2-VL1-L3-VH2-L4-CH1-L5-CL-CH2-CH3, VH2-L1-VL1-L2-VH1-L3-VL2-L4-CL-L5-CH1-CH2-CH3, VH2-L1-VL1-L2-VH1-L3-VL2-L4-CH1-L5- CL-CH2-CH3, VH2-L1-VH1-L2-VL1-L3-VL2-L4-CL-L5-CH1-CH2-CH3, or VH2-L1-VH1-L2-VL1-L3-VL2-L4-CH1-L5-CL-CH2-CH3; and wherein the second polypeptide has a structure represented by VL3-L6-VL4-L7-VH4-L8-VH3-L9-CL-L10-CH1-CH2-CH3, VL3-L6-VL4-L7-VH4-L8-VH3-L9-CH1-L10-CL-CH2-CH3, VL3-L6-VH4-L7-VL4-L8-VH3-L9-CL-L10-CH1-CH2-CH3, VL3-L6-VH4-L7-VL4-L8-VH3-L9-CH1-L10-CL-CH2-CH3, VH3-L6-VL4-L7-VH4-L8-VL3-L9-CL-L10-CH1-CH2-CH3, VH3-L6-VL4- L7-VH4-L8-VL3-L9-CH1-L10-CL-CH2-CH3, VH3-L6-VH4-L7-VL4-L8-VL3-L9-CL-L10-CH1-CH2-CH3, VH3-L6-VH4-L7-VL4-L8-VL3-L9-CH1-L10-CL-CH2-CH3, VL4-L6-VL3-L7-VH3-L8-VH4-L9- CL-L10-CH1-CH2-CH3, VL4-L6-VL3-L7-VH3-L8-VH4-L9-CH1-L10-CL-CH2-CH3, VL4-L6-VH3-L7-VL3-L8-VH4-L9-CL-L10-CH1-CH2-CH3, VL4-L6-VH3-L7-VL3-L8-VH4-L9-CH1-L10-CL-CH2-CH3, VH4- L6-VL3-L7-VH3-L8-VL4-L9-CL-L10-CH1-CH2-CH3, VH4-L6-VL3-L7-VH3-L8-VL4-L9-CH1-L10-CL-CH2-CH3, VH4-L6-VH3-L7-VL3-L8-VL4-L9-CL-L10-CH1-CH2-CH3, or VH4-L6-VH3-L7-VL3-L8-VL4-L9-CH1-L10-CL-CH2-CH3.

In some aspects, an antigen binding polypeptide complex provided herein comprises a first polypeptide, and a second polypeptide; wherein the first polypeptide has a structure represented by VL1-VH1-VL2-CL-VH2-CH1, VL1-VH1-VL2-CH1-VH2-CL, VL1-VH1-VH2-CL-VL2-CH1, VL1-VH1-VH2-CH1-VL2-CL, VL1-VL2-VH1-CL-VH2-CH1, VL1-VL2-VH1-CH1-VH2-CL, VL1-VH2-VH1-CL-VL2-CH1, VL1-VH2-VH1-CH1-VL2-CL, VH1-VL1-VL2-CL-VH2-CH1, VH1-VL1-VL2-CH1-VH2-CL, VH1-VL1-VH2-CL-VL2-CH1, VH1-VL1-VH2-CH1-VL2-CL, VH1-VL2-VL1-CL-VH2-CH1, VH1-VL2-VL1-CH1-VH2-CL, VH1-VH2-VL1-CL-VL2-CH1, VH1-VH2-VL1-CH1-VL2-CL, VL2-VL1-VH2-CL-VH1-CH1, VL2-VL1-VH2-CH1-VH1-CL, VL2-VH1-VH2-CL-VL1-CH1, VL2-VH1-VH2-CH1-VL1-CL, VL2-VH2-VL1-CL-VH1-CH1, VL2-VH2-VL1-CH1-VH1-CL, VL2-VH2-VH1-CL-VL1-CH1, VL2-VH2-VH1-CH1-VL1-CL, VH2-VL1-VL2-CL-VH1-CH1, VH2-VL1-VL2-CH1-VH1-CL, VH2-VH1-VL2-CL-VL1-CH1, VH2-VH1-VL2-CH1-VL1-CL, VH2-VL2-VL1-CL-VH1-CH1, VH2-VL2-VL1-CH1-VH1-CL, VH2-VL2-VH1-CL-VL1-CH1, or VH2-VL2-VH1-CH1-VL1-CL; and wherein the second polypeptide has a structure represented by VL3-CL-VH3-CH1-VL4-VH4, VL3-CL-VH3-CH1-VH4-VL4, VL3-CL-VL4-CH1-VH3-VH4, VL3-CL-VH4-CH1-VH3-VL4, VL3-CH1-VH3-CL-VL4-VH4, VL3-CH1-VH3-CL-VH4-VL4, VL3-CH1-VL4-CL-VH3-VH4, VL3-CH1-VH4-CL-VH3-VL4, VH3-CL-VL3-CH1-VL4-VH4, VH3-CL-VL3-CH1-VH4-VL4, VH3-CL-VL4-CH1-VL3-VH4, VH3-CL-VH4-CH1-VL3-VL4, VH3-CH1-VL3-CL-VL4-VH4, VH3-CH1-VL3-CL-VH4-VL4, VH3-CH1-VL4-CL-VL3-VH4, VH3-CH1-VH4-CL-VL3-VL4, VL4-CL-VL3-CH1-VH4-VH3, VL4-CL-VH3-CH1-VH4-VL3, VL4-CL-VH4-CH1-VL3-VH3, VL4-CL-VH4-CH1-VH3-VL3, VL4-CH1-VL3-CL-VH4-VH3, VL4-CH1-VH3-CL-VH4-VL3, VL4-CH1-VH4-CL-VL3-VH3, VL4-CH1-VH4-CL-VH3-VL3, VH4-CL-VL3-CH1-VL4-VH3, VH4-CL-VH3-CH1-VL4-VL3, VH4-CL-VL4-CH1-VL3-VH3, VH4-CL-VL4-CH1-VH3-VL3, VH4-CH1-VL3-CL-VL4-VH3, VH4-CH1-VH3-CL-VL4-VL3, VH4-CH1-VL4-CL-VL3-VH3, or VH4-CH1-VL4-CL-VH3-VL3.

In some aspects, an antigen binding polypeptide complex provided herein comprises a first polypeptide, and a second polypeptide; wherein the first polypeptide has a structure represented by VL1-L1-VH1-L2-VL2-L3-CL-L4-VH2-L5-CH1, VL1-L1-VH1-L2-VL2-L3-CH1-L4-VH2-L5-CL, VL1-L1-VH1-L2-VH2-L3-CL-L4-VL2-L5-CH1, VL1-L1-VH1-L2-VH2-L3-CH1-L4-VL2-L5-CL, VL1-L1-VL2-L2-VH1-L3-CL-L4-VH2-L5-CH1, VL1-L1-VL2-L2-VH1-L3-CH1-L4-VH2-L5-CL, VL1-L1-VH2-L2-VH1-L3-CL-L4-VL2-L5-CH1, VL1-L1-VH2-L2-VH1-L3-CH1-L4-VL2-L5-CL, VH1-L1-VL1-L2-VL2-L3-CL-L4-VH2-L5-CH1, VH1-L1-VL1-L2-VL2-L3-CH1-L4-VH2-L5-CL, VH1-L1-VL1-L2-VH2-L3-CL-L4-VL2-L5-CH1, VH1-L1-VL1-L2-VH2-L3-CH1-L4-VL2-L5-CL, VH1-L1-VL2-L2-VL1-L3-CL-L4-VH2-L5-CH1, VH1-L1-VL2-L2-VL1-L3-CH1-L4-VH2-L5-CL, VH1-L1-VH2-L2-VL1-L3-CL-L4-VL2-L5-CH1, VH1-L1-VH2-L2-VL1-L3-CH1-L4-VL2-L5-CL, VL2-L1-VL1-L2-VH2-L3-CL-L4-VH1-L5-CH1, VL2-L1-VL1-L2-VH2-L3-CH1-L4-VH1-L5-CL, VL2-L1-VH1-L2-VH2-L3-CL-L4-VL1-L5-CH1, VL2-L1-VH1-L2-VH2-L3-CH1-L4-VL1-L5-CL, VL2-L1-VH2-L2-VL1-L3-CL-L4-VH1-L5-CH1, VL2-L1-VH2-L2-VL1-L3-CH1-L4-VH1-L5-CL, VL2-L1-VH2-L2-VH1-L3-CL-L4-VL1-L5-CH1, VL2-L1-VH2-L2-VH1-L3-CH1-L4-VL1-L5-CL, VH2-L1-VL1-L2-VL2-L3-CL-L4-VH1-L5-CH1, VH2-L1-VL1-L2-VL2-L3-CH1-L4-VH1-L5-CL, VH2-L1-VH1-L2-VL2-L3-CL-L4-VL1-L5-CH1, VH2-L1-VH1-L2-VL2-L3-CH1-L4-VL1-L5-CL, VH2-L1-VL2-L2-VL1-L3-CL-L4-VH1-L5-CH1, VH2-L1-VL2-L2-VL1-L3-CH1-L4-VH1-L5-CL, VH2-L1-VL2-L2-VH1-L3-CL-L4-VL1-L5-CH1, or VH2-L1-VL2-L2-VH1-L3-CH1-L4-VL1-L5-CL; and wherein the second polypeptide has a structure represented by VL3-L6-CL-L7-VH3-L8-CH1-L9-VL4-L10-VH4, VL3-L6-CL-L7-VH3-L8-CH1-L9-VH4-L10-VL4, VL3-L6-CL-L7-VL4-L8-CH1-L9-VH3-L10-VH4, VL3-L6-CL-L7-VH4-L8-CH1-L9-VH3-L10-VL4, VL3-L6-CH1-L7-VH3-L8-CL-L9-VL4-L10-VH4, VL3-L6-CH1-L7-VH3-L8-CL-L9-VH4-L10-VL4, VL3-L6-CH1-L7-VL4-L8-CL-L9-VH3-L10-VH4, VL3-L6-CH1-L7-VH4-L8-CL-L9-VH3-L10-VL4, VH3-L6-CL-L7- VL3-L8-CH1-L9-VL4-L10-VH4, VH3-L6-CL-L7-VL3-L8-CH1-L9-VH4-L10-VL4, VH3-L6-CL-L7-VL4-L8-CH1-L9-VL3-L10-VH4, VH3-L6-CL-L7-VH4-L8-CH1-L9-VL3-L10-VL4, VH3-L6-CH1-L7-VL3-L8-CL-L9-VL4-L10-VH4, VH3-L6-CH1-L7-VL3-L8-CL-L9-VH4-L10-VL4, VH3-L6-CH1-L7-VL4-L8-CL-L9-VL3-L10-VH4, VH3-L6-CH1-L7-VH4-L8-CL-L9-VL3-L10-VL4, VL4-L6-CL-L7-VL3-L8-CH1- L9-VH4-L10-VH3, VL4-L6-CL-L7-VH3-L8-CH1-L9-VH4-L10-VL3, VL4-L6-CL-L7-VH4-L8-CH1-L9-VL3-L10-VH3, VL4-L6-CL-L7-VH4-L8-CH1-L9-VH3-L10-VL3, VL4-L6-CH1-L7-VL3-L8-CL-L9-VH4-L10-VH3, VL4-L6-CH1-L7-VH3-L8-CL-L9-VH4-L10-VL3, VL4-L6-CH1-L7-VH4-L8-CL-L9-VL3-L10-VH3, VL4-L6-CH1-L7-VH4-L8-CL-L9-VH3-L10-VL3, VH4-L6-CL-L7-VL3-L8-CH1-L9-VL4-L10-VH3, VH4-L6-CL-L7-VH3-L8-CH1-L9-VL4-L10-VL3, VH4-L6-CL-L7-VL4-L8-CH1-L9-VL3-L10-VH3, VH4-L6-CL-L7-VL4-L8-CH1-L9-VH3-L10-VL3, VH4-L6-CH1-L7-VL3-L8-CL-L9-VL4-L10-VH3, VH4-L6-CH1-L7-VH3-L8-CL-L9-VL4-L10-VL3, VH4-L6-CH1-L7-VL4-L8-CL-L9-VL3-L10-VH3, or VH4-L6-CH1-L7-VL4-L8-CL-L9-VH3-L10-VL3.

In some aspects, an antigen binding polypeptide complex provided herein comprises a first polypeptide, and a second polypeptide; wherein the first polypeptide has a structure represented by VL1-VH1-VL2-CL-VH2-CH1-CH2-CH3, VL1-VH1-VL2-CH1-VH2-CL-CH2-CH3, VL1-VH1-VH2-CL-VL2-CH1-CH2-CH3, VL1-VH1-VH2-CH1-VL2-CL-CH2-CH3, VL1-VL2-VH1-CL-VH2-CH1-CH2-CH3, VL1-VL2-VH1-CH1-VH2-CL-CH2-CH3, VL1-VH2-VH1-CL-VL2-CH1-CH2-CH3, VL1-VH2-VH1-CH1-VL2-CL-CH2-CH3, VH1-VL1-VL2-CL-VH2-CH1-CH2-CH3, VH1-VL1-VL2-CH1-VH2-CL-CH2-CH3, VH1-VL1-VH2-CL-VL2-CH1-CH2-CH3, VH1-VL1-VH2-CH1-VL2-CL-CH2-CH3, VH1-VL2-VL1-CL-VH2-CH1-CH2-CH3, VH1-VL2-VL1-CH1-VH2-CL-CH2-CH3, VH1-VH2-VL1-CL-VL2-CH1-CH2-CH3, VH1-VH2-VL1-CH1-VL2-CL-CH2-CH3, VL2-VL1-VH2-CL-VH1-CH1-CH2-CH3, VL2-VL1-VH2-CH1-VH1-CL-CH2-CH3, VL2-VH1-VH2-CL-VL1-CH1-CH2-CH3, VL2-VH1-VH2-CH1-VL1-CL-CH2-CH3, VL2-VH2-VL1-CL-VH1-CH1-CH2-CH3, VL2-VH2-VL1-CH1-VH1-CL-CH2-CH3, VL2-VH2-VH1-CL-VL1-CH1-CH2-CH3, VL2-VH2-VH1-CH1-VL1-CL-CH2-CH3, VH2-VL1-VL2-CL-VH1-CH1-CH2-CH3, VH2-VL1-VL2-CH1-VH1-CL-CH2-CH3, VH2-VH1-VL2-CL-VL1-CH1-CH2-CH3, VH2-VH1-VL2-CH1-VL1-CL-CH2-CH3, VH2-VL2-VL1-CL-VH1-CH1-CH2-CH3, VH2-VL2-VL1-CH1-VH1-CL-CH2-CH3, VH2-VL2-VH1-CL-VL1-CH1-CH2-CH3, or VH2-VL2-VH1-CH1-VL1-CL-CH2-CH3; and wherein the second polypeptide has a structure represented by VL3-CL-VH3-CH1-VL4-VH4-CH2-CH3, VL3-CL-VH3-CH1-VH4-VL4-CH2-CH3, VL3-CL-VL4-CH1-VH3-VH4-CH2-CH3, VL3-CL-VH4-CH1-VH3-VL4-CH2-CH3, VL3-CH1-VH3-CL-VL4-VH4-CH2-CH3, VL3-CH1-VH3-CL-VH4-VL4-CH2-CH3, VL3-CH1-VL4-CL-VH3-VH4-CH2-CH3, VL3-CH1-VH4-CL-VH3-VL4-CH2-CH3, VH3-CL-VL3-CH1-VL4-VH4-CH2-CH3, VH3-CL-VL3-CH1-VH4-VL4-CH2-CH3, VH3-CL-VL4-CH1-VL3-VH4-CH2-CH3, VH3-CL-VH4-CH1-VL3-VL4-CH2-CH3, VH3-CH1-VL3-CL-VL4-VH4-CH2-CH3, VH3-CH1-VL3-CL-VH4-VL4-CH2-CH3, VH3-CH1-VL4-CL-VL3-VH4-CH2-CH3, VH3-CH1-VH4-CL-VL3-VL4-CH2-CH3, VL4-CL-VL3-CH1-VH4-VH3-CH2-CH3, VL4-CL-VH3-CH1-VH4-VL3-CH2-CH3, VL4-CL-VH4-CH1-VL3-VH3-CH2-CH3, VL4-CL-VH4-CH1-VH3-VL3-CH2-CH3, VL4-CH1-VL3-CL-VH4-VH3-CH2-CH3, VL4-CH1-VH3-CL-VH4-VL3-CH2-CH3, VL4-CH1-VH4-CL-VL3-VH3-CH2-CH3, VL4-CH1-VH4-CL-VH3-VL3-CH2-CH3, VH4-CL-VL3-CH1-VL4-VH3-CH2-CH3, VH4-CL-VH3-CH1-VL4-VL3-CH2-CH3, VH4-CL-VL4-CH1-VL3-VH3-CH2-CH3, VH4-CL-VL4-CH1-VH3-VL3-CH2-CH3, VH4-CH1-VL3-CL-VL4-VH3-CH2-CH3, VH4-CH1-VH3-CL-VL4-VL3-CH2-CH3, VH4-CH1-VL4-CL-VL3-VH3-CH2-CH3, or VH4-CH1-VL4-CL-VH3-VL3-CH2-CH3.

In some aspects, an antigen binding polypeptide complex provided herein comprises a first polypeptide, and a second polypeptide; wherein the first polypeptide has a structure represented by VL1-L1-VH1-L2-VL2-L3-CL-L4-VH2-L5-CH1-CH2-CH3, VL1-L1-VH1-L2-VL2-L3-CH1-L4-VH2- L5-CL-CH2-CH3, VL1-L1-VH1-L2-VH2-L3-CL-L4-VL2-L5-CH1-CH2-CH3, VL1-L1-VH1-L2-VH2-L3-CH1-L4-VL2-L5-CL-CH2-CH3, VL1-L1-VL2-L2-VH1-L3-CL-L4-VH2-L5-CH1-CH2-CH3, VL1-L1-VL2-L2-VH1-L3-CH1-L4-VH2-L5-CL-CH2-CH3, VL1-L1-VH2-L2-VH1-L3-CL-L4-VL2-L5-CH1-CH2-CH3, VL1-L1-VH2-L2-VH1-L3-CH1-L4-VL2-L5-CL-CH2-CH3, VH1-L1-VL1-L2-VL2-L3-CL-L4-VH2-L5-CH1-CH2-CH3, VH1-L1-VL1-L2-VL2-L3-CH1-L4-VH2-L5-CL-CH2-CH3, VH1-L1-VL1-L2-VH2-L3-CL-L4-VL2-L5-CH1-CH2-CH3, VH1-L1-VL1-L2-VH2-L3-CH1-L4-VL2-L5-CL-CH2-CH3, VH1-L1-VL2-L2-VL1-L3-CL-L4-VH2-L5-CH1-CH2-CH3, VH1-L1-VL2-L2-VL1-L3-CH1-L4-VH2-L5- CL-CH2-CH3, VH1-L1-VH2-L2-VL1-L3-CL-L4-VL2-L5-CH1-CH2-CH3, VH1-L1-VH2-L2-VL1-L3-CH1-L4-VL2-L5-CL-CH2-CH3, VL2-L1-VL1-L2-VH2-L3-CL-L4-VH1-L5-CH1-CH2-CH3, VL2-L1-VL1-L2-VH2-L3-CH1-L4-VH1-L5-CL-CH2-CH3, VL2-L1-VH1-L2-VH2-L3-CL-L4-VL1-L5-CH1-CH2-CH3, VL2-L1-VH1-L2-VH2-L3-CH1-L4-VL1-L5-CL-CH2-CH3, VL2-L1-VH2-L2-VL1-L3-CL-L4-VH1-L5-CH1-CH2-CH3, VL2-L1-VH2-L2-VL1-L3-CH1-L4-VH1-L5-CL-CH2-CH3, VL2-L1-VH2-L2-VH1-L3-CL-L4-VL1-L5-CH1-CH2-CH3, VL2-L1-VH2-L2-VH1-L3-CH1-L4-VL1-L5-CL-CH2-CH3, VH2-L1-VL1-L2-VL2-L3-CL-L4-VH1-L5-CH1-CH2-CH3, VH2-L1-VL1-L2-VL2-L3-CH1-L4-VH1-L5- CL-CH2-CH3, VH2-L1-VH1-L2-VL2-L3-CL-L4-VL1-L5-CH1-CH2-CH3, VH2-L1-VH1-L2-VL2-L3-CH1-L4-VL1-L5-CL-CH2-CH3, VH2-L1-VL2-L2-VL1-L3-CL-L4-VH1-L5-CH1-CH2-CH3, VH2-L1-VL2-L2-VL1-L3-CH1-L4-VH1-L5-CL-CH2-CH3, VH2-L1-VL2-L2-VH1-L3-CL-L4-VL1-L5-CH1-CH2-CH3, or VH2-L1-VL2-L2-VH1-L3-CH1-L4-VL1-L5-CL-CH2-CH3; and wherein the second polypeptide has a structure represented by VL3-L6-CL-L7-VH3-L8-CH1-L9-VL4-L10-VH4-CH2-CH3, VL3-L6-CL-L7-VH3-L8-CH1-L9-VH4-L10-VL4-CH2-CH3, VL3-L6-CL-L7-VL4-L8-CH1-L9-VH3-L10-VH4-CH2-CH3, VL3-L6-CL-L7-VH4-L8-CH1-L9-VH3-L10-VL4-CH2-CH3, VL3-L6-CH1-L7-VH3-L8-CL-L9-VL4-L10-VH4-CH2-CH3, VL3-L6-CH1-L7-VH3-L8-CL-L9-VH4-L10-VL4-CH2-CH3, VL3-L6-CH1-L7-VL4-L8-CL-L9-VH3-L10-VH4-CH2-CH3, VL3-L6-CH1-L7-VH4-L8-CL-L9-VH3-L10-VL4-CH2-CH3, VH3- L6-CL-L7-VL3-L8-CH1-L9-VL4-L10-VH4-CH2-CH3, VH3-L6-CL-L7-VL3-L8-CH1-L9-VH4-L10-VL4-CH2-CH3, VH3-L6-CL-L7-VL4-L8-CH1-L9-VL3-L10-VH4-CH2-CH3, VH3-L6-CL-L7-VH4-L8-CH1-L9-VL3-L10-VL4-CH2-CH3, VH3-L6-CH1-L7-VL3-L8-CL-L9-VL4-L10-VH4-CH2-CH3, VH3-L6-CH1-L7-VL3-L8-CL-L9-VH4-L10-VL4-CH2-CH3, VH3-L6-CH1-L7-VL4-L8-CL-L9-VL3-L10-VH4-CH2-CH3, VH3-L6-CH1-L7-VH4-L8-CL-L9-VL3-L10-VL4-CH2-CH3, VL4-L6-CL-L7-VL3-L8-CH1-L9-VH4-L10-VH3-CH2-CH3, VL4-L6-CL-L7-VH3-L8-CH1-L9-VH4-L10-VL3-CH2-CH3, VL4-L6-CL- L7-VH4-L8-CH1-L9-VL3-L10-VH3-CH2-CH3, VL4-L6-CL-L7-VH4-L8-CH1-L9-VH3-L10-VL3-CH2-CH3, VL4-L6-CH1-L7-VL3-L8-CL-L9-VH4-L10-VH3-CH2-CH3, VL4-L6-CH1-L7-VH3-L8-CL-L9-VH4-L10-VL3-CH2-CH3, VL4-L6-CH1-L7-VH4-L8-CL-L9-VL3-L10-VH3-CH2-CH3, VL4-L6-CH1-L7-VH4-L8-CL-L9-VH3-L10-VL3-CH2-CH3, VH4-L6-CL-L7-VL3-L8-CH1-L9-VL4-L10-VH3-CH2-CH3, VH4- L6-CL-L7-VH3-L8-CH1-L9-VL4-L10-VL3-CH2-CH3, VH4-L6-CL-L7-VL4-L8-CH1-L9-VL3-L10-VH3-CH2-CH3, VH4-L6-CL-L7-VL4-L8-CH1-L9-VH3-L10-VL3-CH2-CH3, VH4-L6-CH1-L7-VL3-L8-CL-L9-VL4-L10-VH3-CH2-CH3, VH4-L6-CH1-L7-VH3-L8-CL-L9-VL4-L10-VL3-CH2-CH3, VH4-L6-CH1-L7-VL4-L8-CL-L9-VL3-L10-VH3-CH2-CH3, or VH4-L6-CH1-L7-VL4-L8-CL-L9-VH3-L10- VL3-CH2-CH3.

In some aspects, an antigen binding polypeptide complex provided herein comprises a first polypeptide, and a second polypeptide; wherein the first polypeptide has a structure represented by VL1-CL-VH1-CH1-VL2-VH2, VL1-CL-VH1-CH1-VH2-VL2, VL1-CL-VL2-CH1-VH1-VH2, VL1-CL-VH2-CH1-VH1-VL2, VL1-CH1-VH1-CL-VL2-VH2, VL1-CH1-VH1-CL-VH2-VL2, VL1-CH1-VL2-CL-VH1-VH2, VL1-CH1-VH2-CL-VH1-VL2, VH1-CL-VL1-CH1-VL2-VH2, VH1-CL-VL1-CH1-VH2-VL2, VH1-CL-VL2-CH1-VL1-VH2, VH1-CL-VH2-CH1-VL1-VL2, VH1-CH1-VL1-CL-VL2-VH2, VH1-CH1-VL1-CL-VH2-VL2, VH1-CH1-VL2-CL-VL1-VH2, VH1-CH1-VH2-CL-VL1-VL2, VL2-CL-VL1-CH1-VH2-VH1, VL2-CL-VH1-CH1-VH2-VL1, VL2-CL-VH2-CH1-VL1-VH1, VL2-CL-VH2-CH1-VH1-VL1, VL2-CH1-VL1-CL-VH2-VH1, VL2-CH1-VH1-CL-VH2-VL1, VL2-CH1-VH2-CL-VL1-VH1, VL2-CH1-VH2-CL-VH1-VL1, VH2-CL-VL1-CH1-VL2-VH1, VH2-CL-VH1-CH1-VL2-VL1, VH2-CL-VL2-CH1-VL1-VH1, VH2-CL-VL2-CH1-VH1-VL1, VH2-CH1-VL1-CL-VL2-VH1, VH2-CH1-VH1-CL-VL2-VL1, VH2-CH1-VL2-CL-VL1-VH1, or VH2-CH1-VL2-CL-VH1-VL1; and wherein the second polypeptide has a structure represented by VL3-VH3-VL4-CL-VH4-CH1, VL3-VH3-VL4-CH1-VH4-CL, VL3-VH3-VH4-CL-VL4-CH1, VL3-VH3-VH4-CH1-VL4-CL, VL3-VL4-VH3-CL-VH4-CH1, VL3-VL4-VH3-CH1-VH4-CL, VL3-VH4-VH3-CL-VL4-CH1, VL3-VH4-VH3-CH1-VL4-CL, VH3-VL3-VL4-CL-VH4-CH1, VH3-VL3-VL4-CH1-VH4-CL, VH3-VL3-VH4-CL-VL4-CH1, VH3-VL3-VH4-CH1-VL4-CL, VH3-VL4-VL3-CL-VH4-CH1, VH3-VL4-VL3-CH1-VH4-CL, VH3-VH4-VL3-CL-VL4-CH1, VH3-VH4-VL3-CH1-VL4-CL, VL4-VL3-VH4-CL-VH3-CH1, VL4-VL3-VH4-CH1-VH3-CL, VL4-VH3-VH4-CL-VL3-CH1, VL4-VH3-VH4-CH1-VL3-CL, VL4-VH4-VL3-CL-VH3-CH1, VL4-VH4-VL3-CH1-VH3-CL, VL4-VH4-VH3-CL-VL3-CH1, VL4-VH4-VH3-CH1-VL3-CL, VH4-VL3-VL4-CL-VH3-CH1, VH4-VL3-VL4-CH1-VH3-CL, VH4-VH3-VL4-CL-VL3-CH1, VH4-VH3-VL4-CH1-VL3-CL, VH4-VL4-VL3-CL-VH3-CH1, VH4-VL4-VL3-CH1-VH3-CL, VH4-VL4-VH3-CL-VL3-CH1, or VH4-VL4-VH3-CH1-VL3-CL.

In some aspects, an antigen binding polypeptide complex provided herein comprises a first polypeptide, and a second polypeptide; wherein the first polypeptide has a structure represented by VL1-L1-CL-L2-VH1-L3-CH1-L4-VL2-L5-VH2, VL1-L1-CL-L2-VH1-L3-CH1-L4-VH2-L5-VL2, VL1-L1-CL-L2-VL2-L3-CH1-L4-VH1-L5-VH2, VL1-L1-CL-L2-VH2-L3-CH1-L4-VH1-L5-VL2, VL1-L1-CH1-L2-VH1-L3-CL-L4-VL2-L5-VH2, VL1-L1-CH1-L2-VH1-L3-CL-L4-VH2-L5-VL2, VL1-L1-CH1-L2-VL2-L3-CL-L4-VH1-L5-VH2, VL1-L1-CH1-L2-VH2-L3-CL-L4-VH1-L5-VL2, VH1-L1-CL-L2-VL1-L3-CH1-L4-VL2-L5-VH2, VH1-L1-CL-L2-VL1-L3-CH1-L4-VH2-L5-VL2, VH1-L1-CL-L2-VL2-L3-CH1-L4-VL1-L5-VH2, VH1-L1-CL-L2-VH2-L3-CH1-L4-VL1-L5-VL2, VH1-L1-CH1-L2-VL1-L3- CL-L4-VL2-L5-VH2, VH1-L1-CH1-L2-VL1-L3-CL-L4-VH2-L5-VL2, VH1-L1-CH1-L2-VL2-L3-CL-L4-VL1-L5-VH2, VH1-L1-CH1-L2-VH2-L3-CL-L4-VL1-L5-VL2, VL2-L1-CL-L2-VL1-L3-CH1-L4-VH2-L5-VH1, VL2-L1-CL-L2-VH1-L3-CH1-L4-VH2-L5-VL1, VL2-L1-CL-L2-VH2-L3-CH1-L4-VL1-L5-VH1, VL2-L1-CL-L2-VH2-L3-CH1-L4-VH1-L5-VL1, VL2-L1-CH1-L2-VL1-L3-CL-L4-VH2-L5-VH1, VL2-L1-CH1-L2-VH1-L3-CL-L4-VH2-L5-VL1, VL2-L1-CH1-L2-VH2-L3-CL-L4-VL1-L5-VH1, VL2-L1-CH1-L2-VH2-L3-CL-L4-VH1-L5-VL1, VH2-L1-CL-L2-VL1-L3-CH1-L4-VL2-L5-VH1, VH2-L1-CL-L2-VH1-L3-CH1-L4-VL2-L5-VL1, VH2-L1-CL-L2-VL2-L3-CH1-L4-VL1-L5-VH1, VH2-L1-CL- L2-VL2-L3-CH1-L4-VH1-L5-VL1, VH2-L1-CH1-L2-VL1-L3-CL-L4-VL2-L5-VH1, VH2-L1-CH1-L2-VH1-L3-CL-L4-VL2-L5-VL1, VH2-L1-CH1-L2-VL2-L3-CL-L4-VL1-L5-VH1, or VH2-L1-CH1-L2-VL2-L3-CL-L4-VH1-L5-VL1; and wherein the second polypeptide has a structure represented by VL3-L6-VH3-L7-VL4-L8-CL-L9-VH4-L10-CH1, VL3-L6-VH3-L7-VL4-L8-CH1-L9-VH4-L10-CL, VL3-L6-VH3-L7-VH4-L8-CL-L9-VL4-L10-CH1, VL3-L6-VH3-L7-VH4-L8-CH1-L9-VL4-L10-CL, VL3-L6-VL4-L7-VH3-L8-CL-L9-VH4-L10-CH1, VL3-L6-VL4-L7-VH3-L8-CH1-L9-VH4-L10-CL, VL3-L6-VH4-L7-VH3-L8-CL-L9-VL4-L10-CH1, VL3-L6-VH4-L7-VH3-L8-CH1-L9-VL4-L10-CL, VH3-L6-VL3-L7-VL4-L8-CL-L9-VH4-L10-CH1, VH3-L6-VL3-L7-VL4-L8-CH1-L9-VH4-L10-CL, VH3-L6-VL3-L7-VH4-L8-CL-L9-VL4-L10-CH1, VH3-L6-VL3-L7-VH4-L8-CH1-L9-VL4-L10-CL, VH3-L6-VL4-L7-VL3-L8-CL-L9-VH4-L10-CH1, VH3-L6-VL4-L7-VL3-L8-CH1-L9-VH4-L10-CL, VH3-L6-VH4-L7-VL3-L8-CL-L9-VL4-L10-CH1, VH3-L6-VH4-L7-VL3-L8-CH1-L9-VL4-L10-CL, VL4-L6-VL3-L7-VH4-L8-CL-L9-VH3-L10-CH1, VL4-L6-VL3-L7-VH4-L8-CH1-L9-VH3-L10-CL, VL4-L6-VH3-L7-VH4-L8-CL-L9-VL3-L10-CH1, VL4-L6-VH3-L7-VH4-L8-CH1-L9-VL3-L10-CL, VL4-L6-VH4-L7-VL3-L8-CL-L9-VH3-L10-CH1, VL4-L6-VH4-L7-VL3-L8-CH1-L9-VH3-L10-CL, VL4-L6-VH4-L7-VH3-L8-CL- L9-VL3-L10-CH1, VL4-L6-VH4-L7-VH3-L8-CH1-L9-VL3-L10-CL, VH4-L6-VL3-L7-VL4-L8-CL-L9-VH3-L10-CH1, VH4-L6-VL3-L7-VL4-L8-CH1-L9-VH3-L10-CL, VH4-L6-VH3-L7-VL4-L8-CL-L9-VL3-L10-CH1, VH4-L6-VH3-L7-VL4-L8-CH1-L9-VL3-L10-CL, VH4-L6-VL4-L7-VL3-L8-CL-L9-VH3-L10-CH1, VH4-L6-VL4-L7-VL3-L8-CH1-L9-VH3-L10-CL, VH4-L6-VL4-L7-VH3-L8-CL-L9-VL3-L10-CH1, or VH4-L6-VL4-L7-VH3-L8-CH1-L9-VL3-L10-CL.

In some aspects, an antigen binding polypeptide complex provided herein comprises a first polypeptide, and a second polypeptide; wherein the first polypeptide has a structure represented by VL1-CL-VH1-CH1-VL2-VH2-CH2-CH3, VL1-CL-VH1-CH1-VH2-VL2-CH2-CH3, VL1-CL-VL2-CH1-VH1-VH2-CH2-CH3, VL1-CL-VH2-CH1-VH1-VL2-CH2-CH3, VL1-CH1-VH1-CL-VL2-VH2-CH2-CH3, VL1-CH1-VH1-CL-VH2-VL2-CH2-CH3, VL1-CH1-VL2-CL-VH1-VH2-CH2-CH3, VL1-CH1-VH2-CL-VH1-VL2-CH2-CH3, VH1-CL-VL1-CH1-VL2-VH2-CH2-CH3, VH1-CL-VL1-CH1-VH2-VL2-CH2-CH3, VH1-CL-VL2-CH1-VL1-VH2-CH2-CH3, VH1-CL-VH2-CH1-VL1-VL2-CH2-CH3, VH1-CH1-VL1-CL-VL2-VH2-CH2-CH3, VH1-CH1-VL1-CL-VH2-VL2-CH2-CH3, VH1-CH1-VL2-CL-VL1-VH2-CH2-CH3, VH1-CH1-VH2-CL-VL1-VL2-CH2-CH3, VL2-CL-VL1-CH1-VH2-VH1-CH2-CH3, VL2-CL-VH1-CH1-VH2-VL1-CH2-CH3, VL2-CL-VH2-CH1-VL1-VH1-CH2-CH3, VL2-CL-VH2-CH1-VH1-VL1-CH2-CH3, VL2-CH1-VL1-CL-VH2-VH1-CH2-CH3, VL2-CH1-VH1-CL-VH2-VL1-CH2-CH3, VL2-CH1-VH2-CL-VL1-VH1-CH2-CH3, VL2-CH1-VH2-CL-VH1-VL1-CH2-CH3, VH2-CL-VL1-CH1-VL2-VH1-CH2-CH3, VH2-CL-VH1-CH1-VL2-VL1-CH2-CH3, VH2-CL-VL2-CH1-VL1-VH1-CH2-CH3, VH2-CL-VL2-CH1-VH1-VL1-CH2-CH3, VH2-CH1-VL1-CL-VL2-VH1-CH2-CH3, VH2-CH1-VH1-CL-VL2-VL1-CH2-CH3, VH2-CH1-VL2-CL-VL1-VH1-CH2-CH3, or VH2-CH1-VL2-CL-VH1-VL1-CH2-CH3; and wherein the second polypeptide has a structure represented by VL3-VH3-VL4-CL-VH4-CH1-CH2-CH3, VL3-VH3-VL4-CH1-VH4-CL-CH2-CH3, VL3-VH3-VH4-CL-VL4-CH1-CH2-CH3, VL3-VH3-VH4-CH1-VL4-CL-CH2-CH3, VL3-VL4-VH3-CL-VH4-CH1-CH2-CH3, VL3-VL4-VH3-CH1-VH4-CL-CH2-CH3, VL3-VH4-VH3-CL-VL4-CH1-CH2-CH3, VL3-VH4-VH3-CH1-VL4-CL-CH2-CH3, VH3-VL3-VL4-CL-VH4-CH1-CH2-CH3, VH3-VL3-VL4-CH1-VH4-CL-CH2-CH3, VH3-VL3-VH4-CL-VL4-CH1-CH2-CH3, VH3-VL3-VH4-CH1-VL4-CL-CH2-CH3, VH3-VL4-VL3-CL-VH4-CH1-CH2-CH3, VH3-VL4-VL3-CH1-VH4-CL-CH2-CH3, VH3-VH4-VL3-CL-VL4-CH1-CH2-CH3, VH3-VH4-VL3-CH1-VL4-CL-CH2-CH3, VL4-VL3-VH4-CL-VH3-CH1-CH2-CH3, VL4-VL3-VH4-CH1-VH3-CL-CH2-CH3, VL4-VH3-VH4-CL-VL3-CH1-CH2-CH3, VL4-VH3-VH4-CH1-VL3-CL-CH2-CH3, VL4-VH4-VL3-CL-VH3-CH1-CH2-CH3, VL4-VH4-VL3-CH1-VH3-CL-CH2-CH3, VL4-VH4-VH3-CL-VL3-CH1-CH2-CH3, VL4-VH4-VH3-CH1-VL3-CL-CH2-CH3, VH4-VL3-VL4-CL-VH3-CH1-CH2-CH3, VH4-VL3-VL4-CH1-VH3-CL-CH2-CH3, VH4-VH3-VL4-CL-VL3-CH1-CH2-CH3, VH4-VH3-VL4-CH1-VL3-CL-CH2-CH3, VH4-VL4-VL3-CL-VH3-CH1-CH2-CH3, VH4-VL4-VL3-CH1-VH3-CL-CH2-CH3, VH4-VL4-VH3-CL-VL3-CH1-CH2-CH3, or VH4-VL4-VH3-CH1-VL3-CL-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-L4-VL2-L5-VH2-CH2-CH3, VL1-L1-CL-L2-VH1-L3-CH1-L4-VH2-L5-VL2-CH2-CH3, VL1-L1-CL-L2-VL2-L3-CH1-L4-VH1-L5-VH2-CH2-CH3, VL1-L1-CL-L2-VH2-L3-CH1-L4-VH1-L5-VL2-CH2-CH3, VL1-L1-CH1-L2-VH1-L3-CL-L4-VL2-L5-VH2-CH2-CH3, VL1-L1-CH1-L2-VH1-L3-CL-L4-VH2-L5-VL2-CH2-CH3, VL1-L1-CH1-L2-VL2-L3-CL-L4-VH1-L5-VH2-CH2-CH3, VL1-L1-CH1-L2-VH2-L3-CL-L4-VH1-L5-VL2-CH2-CH3, VH1-L1-CL-L2-VL1-L3-CH1-L4-VL2-L5-VH2-CH2-CH3, VH1-L1-CL-L2-VL1-L3-CH1-L4-VH2-L5-VL2-CH2-CH3, VH1-L1-CL-L2-VL2-L3-CH1-L4-VL1-L5-VH2-CH2-CH3, VH1-L1-CL-L2-VH2-L3-CH1-L4-VL1-L5-VL2-CH2-CH3, VH1-L1-CH1-L2-VL1-L3-CL-L4-VL2-L5-VH2-CH2-CH3, VH1-L1-CH1-L2-VL1-L3-CL-L4-VH2-L5-VL2-CH2-CH3, VH1-L1-CH1-L2-VL2-L3-CL-L4-VL1-L5-VH2-CH2-CH3, VH1-L1-CH1-L2-VH2-L3-CL-L4-VL1-L5-VL2-CH2-CH3, VL2-L1-CL-L2-VL1-L3-CH1-L4-VH2-L5-VH1-CH2-CH3, VL2-L1-CL-L2-VH1-L3-CH1-L4-VH2-L5-VL1-CH2-CH3, VL2-L1-CL-L2-VH2-L3-CH1-L4-VL1-L5-VH1-CH2-CH3, VL2-L1-CL-L2-VH2-L3-CH1-L4-VH1-L5-VL1-CH2-CH3, VL2-L1-CH1-L2-VL1-L3-CL-L4-VH2-L5-VH1-CH2-CH3, VL2-L1-CH1-L2-VH1-L3-CL-L4-VH2-L5-VL1-CH2-CH3, VL2-L1-CH1-L2-VH2-L3-CL-L4-VL1-L5-VH1-CH2-CH3, VL2-L1-CH1-L2-VH2-L3-CL-L4-VH1-L5-VL1-CH2-CH3, VH2-L1-CL-L2-VL1-L3-CH1-L4-VL2-L5-VH1-CH2-CH3, VH2-L1-CL-L2-VH1-L3-CH1-L4-VL2-L5-VL1-CH2-CH3, VH2-L1-CL-L2-VL2-L3-CH1-L4-VL1-L5-VH1-CH2-CH3, VH2-L1-CL-L2-VL2-L3-CH1-L4-VH1-L5-VL1-CH2-CH3, VH2-L1-CH1-L2-VL1-L3-CL-L4-VL2-L5-VH1-CH2-CH3, VH2-L1-CH1-L2-VH1-L3-CL-L4-VL2-L5-VL1-CH2-CH3, VH2-L1-CH1-L2-VL2-L3-CL-L4-VL1-L5-VH1-CH2-CH3, or VH2-L1-CH1-L2-VL2-L3-CL-L4-VH1-L5-VL1-CH2-CH3; and wherein the second polypeptide has a structure represented by VL3-L6-VH3-L7-VL4-L8-CL-L9-VH4-L10-CH1-CH2-CH3, VL3-L6-VH3-L7-VL4-L8-CH1-L9-VH4-L10-CL-CH2-CH3, VL3-L6-VH3-L7-VH4-L8-CL-L9-VL4-L10-CH1-CH2-CH3, VL3-L6-VH3-L7- VH4-L8-CH1-L9-VL4-L10-CL-CH2-CH3, VL3-L6-VL4-L7-VH3-L8-CL-L9-VH4-L10-CH1-CH2-CH3, VL3-L6-VL4-L7-VH3-L8-CH1-L9-VH4-L10-CL-CH2-CH3, VL3-L6-VH4-L7-VH3-L8-CL-L9-VL4-L10-CH1-CH2-CH3, VL3-L6-VH4-L7-VH3-L8-CH1-L9-VL4-L10-CL-CH2-CH3, VH3-L6-VL3-L7-VL4-L8-CL-L9-VH4-L10-CH1-CH2-CH3, VH3-L6-VL3-L7-VL4-L8-CH1-L9-VH4-L10-CL-CH2-CH3, VH3-L6-VL3-L7-VH4-L8-CL-L9-VL4-L10-CH1-CH2-CH3, VH3-L6-VL3-L7-VH4-L8-CH1-L9-VL4-L10-CL-CH2-CH3, VH3-L6-VL4-L7-VL3-L8-CL-L9-VH4-L10-CH1-CH2-CH3, VH3-L6-VL4-L7-VL3-L8- CH1-L9-VH4-L10-CL-CH2-CH3, VH3-L6-VH4-L7-VL3-L8-CL-L9-VL4-L10-CH1-CH2-CH3, VH3-L6- VH4-L7-VL3-L8-CH1-L9-VL4-L10-CL-CH2-CH3, VL4-L6-VL3-L7-VH4-L8-CL-L9-VH3-L10-CH1-CH2-CH3, VL4-L6-VL3-L7-VH4-L8-CH1-L9-VH3-L10-CL-CH2-CH3, VL4-L6-VH3-L7-VH4-L8-CL-L9-VL3-L10-CH1-CH2-CH3, VL4-L6-VH3-L7-VH4-L8-CH1-L9-VL3-L10-CL-CH2-CH3, VL4-L6-VH4-L7-VL3-L8-CL-L9-VH3-L10-CH1-CH2-CH3, VL4-L6-VH4-L7-VL3-L8-CH1-L9-VH3-L10-CL-CH2-CH3, VL4-L6-VH4-L7-VH3-L8-CL-L9-VL3-L10-CH1-CH2-CH3, VL4-L6-VH4-L7-VH3-L8-CH1-L9- VL3-L10-CL-CH2-CH3, VH4-L6-VL3-L7-VL4-L8-CL-L9-VH3-L10-CH1-CH2-CH3, VH4-L6-VL3-L7- VL4-L8-CH1-L9-VH3-L10-CL-CH2-CH3, VH4-L6-VH3-L7-VL4-L8-CL-L9-VL3-L10-CH1-CH2-CH3, VH4-L6-VH3-L7-VL4-L8-CH1-L9-VL3-L10-CL-CH2-CH3, VH4-L6-VL4-L7-VL3-L8-CL-L9-VH3-L10-CH1-CH2-CH3, VH4-L6-VL4-L7-VL3-L8-CH1-L9-VH3-L10-CL-CH2-CH3, VH4-L6-VL4-L7-VH3-L8-CL-L9-VL3-L10-CH1-CH2-CH3, or VH4-L6-VL4-L7-VH3-L8-CH1-L9-VL3-L10-CL-CH2-CH3.

In some aspects, VL1 is a first immunoglobulin light chain variable region that specifically binds to a SARS-CoV-2 protein (e.g., a SARS-CoV-2 spike protein); VL2 is a second immunoglobulin light chain variable region that specifically binds to a SARS-CoV-2 protein (e.g., a SARS-CoV-2 spike protein); VL3 is a third immunoglobulin light chain variable region that specifically binds to a SARS-CoV-2 protein (e.g., a SARS-CoV-2 spike protein); VL4 is a fourth immunoglobulin light chain variable region that specifically binds to a SARS-CoV-2 protein (e.g., a SARS-CoV-2 spike protein); VH1 is a first immunoglobulin heavy chain variable region that specifically binds to a SARS-CoV-2 protein (e.g., a SARS-CoV-2 spike protein); VH2 is a second immunoglobulin heavy chain variable region that specifically binds to a SARS-CoV-2 protein (e.g., a SARS-CoV-2 spike protein); VH3 is a third immunoglobulin heavy chain variable region that specifically binds to a SARS-CoV-2 protein (e.g., a SARS-CoV-2 spike protein); VH4 is a fourth immunoglobulin heavy chain variable region that specifically binds to a SARS-CoV-2 protein (e.g., a SARS-CoV-2 spike protein); CH1 is an immunoglobulin heavy chain constant region 1; CL is an immunoglobulin light chain constant region; and L1-L10 are optional amino acid linkers. In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the VL1 and the VL2 each comprise a CDR1, a CDR2, and a CDR3 that are the same. In some aspects, the VL1 and the VL2 each comprise a CDR1, a CDR2, and a CDR3 that are different. In some aspects, the VL1 and the VL3 each comprise a CDR1, a CDR2, and a CDR3 that are the same. In some aspects, the VL1 and the VL3 each comprise a CDR1, a CDR2, and a CDR3 that are different. In some aspects, the VL1 and the VL4 each comprise a CDR1, a CDR2, and a CDR3 that are the same. In some aspects, the VL1 and the VL4 each comprise a CDR1, a CDR2, and a CDR3 that are different. In some aspects, the VL2 and the VL3 each comprise a CDR1, a CDR2, and a CDR3 that are the same. In some aspects, the VL2 and the VL3 each comprise a CDR1, a CDR2, and a CDR3 that are different. In some aspects, the VL2 and the VL4 each comprise a CDR1, a CDR2, and a CDR3 that are the same. In some aspects, the VL2 and the VL4 each comprise a CDR1, a CDR2, and a CDR3 that are different. In some aspects, the VL3 and the VL4 each comprise a CDR1, a CDR2, and a CDR3 that are the same. In some aspects, the VL3 and the VL4 each comprise a CDR1, a CDR2, and a CDR3 that are different.

In some aspects, the VL1 and the VL2 each comprise an amino acid sequence, wherein the amino acid sequence comprised in VL1 and the amino acid sequence comprised in VL2 are the same. In some aspects, the VL1 and the VL2 each comprise an amino acid sequence, wherein the amino acid sequence comprised in VL1 and the amino acid sequence comprised in VL2 are different. In some aspects, the VL1 and the VL3 each comprise an amino acid sequence, wherein the amino acid sequence comprised in VL1 and the amino acid sequence comprised in VL3 are the same. In some aspects, the VL1 and the VL3 each comprise an amino acid sequence, wherein the amino acid sequence comprised in VL1 and the amino acid sequence comprised in VL3 are different. In some aspects, the VL1 and the VL4 each comprise an amino acid sequence, wherein the amino acid sequence comprised in VL1 and the amino acid sequence comprised in VL4 are the same. In some aspects, the VL1 and the VL4 each comprise an amino acid sequence, wherein the amino acid sequence comprised in VL1 and the amino acid sequence comprised in VL4 are different. In some aspects, the VL2 and the VL3 each comprise an amino acid sequence, wherein the amino acid sequence comprised in VL2 and the amino acid sequence comprised in VL3 are the same. In some aspects, the VL2 and the VL3 each comprise an amino acid sequence, wherein the amino acid sequence comprised in VL2 and the amino acid sequence comprised in VL3 are different. In some aspects, the VL2 and the VL4 each comprise an amino acid sequence, wherein the amino acid sequence comprised in VL2 and the amino acid sequence comprised in VL4 are the same. In some aspects, the VL2 and the VL4 each comprise an amino acid sequence, wherein the amino acid sequence comprised in VL2 and the amino acid sequence comprised in VL4 are different. In some aspects, the VL3 and the VL4 each comprise an amino acid sequence, wherein the amino acid sequence comprised in VL3 and the amino acid sequence comprised in VL4 are the same. In some aspects, the VL3 and the VL4 each comprise an amino acid sequence, wherein the amino acid sequence comprised in VL3 and the amino acid sequence comprised in VL4 are different.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 95% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 95% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 95% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765 767, 143, 857, 177, 793, 1049, 1054, 1059, 1064, and 1069.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069. In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL3 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL3 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL3 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL3 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL3 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 71, 764, 766, 768, 771, 779, 786, 792, 795, 808, 815, 821, 830, 837, 843, 850, 856, 859, 866, 872, 879, and 885.

In some aspects, VL3 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066.

In some aspects, VL3 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792.

In some aspects, VL3 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL3 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH3 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH3 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH3 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH3 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH3 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH3 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069.

In some aspects, VH3 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064.

In some aspects, VH3 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH3 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL4 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL4 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL4 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL4 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL4 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL4 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066.

In some aspects, VL4 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792.

In some aspects, VL4 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL4 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH4 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH4 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH4 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH4 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH4 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH4 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054. In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857 or 1069.

In some aspects, VH4 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064.

In some aspects, VH4 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH4 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, at least one of VL1, VL2, VL3, or VL4 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1, VH2, VH3, or VH4 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1, VL2, VL3, or VL4 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1, VH2, VH3, or VH4 comprised in the antigen binding polypeptide complexes provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1, VL2, VL3, or VL4 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1, VH2, VH3, or VH4 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1, VL2, VL3, or VL4 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1, VH2, VH3, or VH4 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL3 comprising (xiii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (xiv) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (xv) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH3 comprising (xvi) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xvii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xviii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL4 comprising (xix) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (xx) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (xxi) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH4 comprising (xxii) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xxiii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (xxiv) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (v) a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (vi) a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (vii) a VL4 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (viii) a VH4 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; a VL3 comprising (xiii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (xiv) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (xv) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; a VH3 comprising (xvi) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xvii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xviii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; a VL4 comprising (xix) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (xx) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (xxi) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH4 comprising (xxii) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xxiii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (xxiv) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (v) a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (vi) a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (vii) a VL4 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (viii) a VH4 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 138, 172, 1060 or 1065; (viii) a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS; and (ix) a CDR3 having an amino acid sequence at least 65%, at least 70%, at least 75%, at least 80%, at least 85%, at least 90%, at least 95%, at least 99%, or 100% identical to SEQ ID NO: 139, 856, 173, 792, or 1066; a VH2 comprising (x) a CDR1 having an amino acid sequence at least 65%, at least 70%, at least 75%, at least 80%, at least 85%, at least 90%, at least 95%, at least 99%, or 100% identical to SEQ ID NO: 141, 175 1062, or 1067; (xi) a CDR2 having an amino acid sequence at least 65%, at least 70%, at least 75%, at least 80%, at least 85%, at least 90%, at least 95%, at least 99%, or 100% identical to SEQ ID NO: 142, 176, 1063, or 1068; and (xii) a CDR3 having an amino acid sequence at least 65%, at least 70%, at least 75%, at least 80%, at least 85%, at least 90%, at least 95%, at least 99%, or 100% identical to SEQ ID NO: 143, 857, 177, 793, 1064, or 1069; a VL3 comprising (xiii) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 138, 172, 1060 or 1065; (xiv) a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS; and (xv) a CDR3 having an amino acid sequence at least 65%, at least 70%, at least 75%, at least 80%, at least 85%, at least 90%, at least 95%, at least 99%, or 100% identical to SEQ ID NO: 139, 856, 173, 792, or 1066; a VH3 comprising (xvi) a CDR1 having an amino acid sequence at least 65%, at least 70%, at least 75%, at least 80%, at least 85%, at least 90%, at least 95%, at least 99%, or 100% identical to SEQ ID NO: 141, 175 1062, or 1067; (xvii) a CDR2 having an amino acid sequence at least 65%, at least 70%, at least 75%, at least 80%, at least 85%, at least 90%, at least 95%, at least 99%, or 100% identical to SEQ ID NO: 142, 176, 1063, or 1068; and (xviii) a CDR3 having an amino acid sequence at least 65%, at least 70%, at least 75%, at least 80%, at least 85%, at least 90%, at least 95%, at least 99%, or 100% identical to SEQ ID NO: 143, 857, 177, 793, 1064, or 1069; a VL4 comprising (xix) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 138, 172, 1060 or 1065; (xx) a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS; and; and (xxi) a CDR3 having an amino acid sequence at least 65%, at least 70%, at least 75%, at least 80%, at least 85%, at least 90%, at least 95%, at least 99%, or 100% identical to SEQ ID NO: 139, 856, 173, 792, or 1066; a VH2 comprising; and a VH4 comprising (xxii) a CDR1 having an amino acid sequence at least 65%, at least 70%, at least 75%, at least 80%, at least 85%, at least 90%, at least 95%, at least 99%, or 100% identical to SEQ ID NO: 141, 175 1062, or 1067; (xxiii) a CDR2 having an amino acid sequence at least 65%, at least 70%, at least 75%, at least 80%, at least 85%, at least 90%, at least 95%, at least 99%, or 100% identical to SEQ ID NO: 142, 176, 1063, or 1068; and (xxiv) a CDR3 having an amino acid sequence at least 65%, at least 70%, at least 75%, at least 80%, at least 85%, at least 90%, at least 95%, at least 99%, or 100% identical to SEQ ID NO: 143, 857, 177, 793, 1064, or 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to SEQ ID NO: 140 or 174; (iv) a VH2 comprising an amino acid sequence that is at least 65%, at least 70%, at least 75%, at least 80%, at least 85%, at least 90%, at least 95%, at least 99%, or 100% identical to SEQ ID NO: 144 or 178; (v) a VL3 comprising an amino acid sequence that is at least 65%, at least 70%, at least 75%, at least 80%, at least 85%, at least 90%, at least 95%, at least 99%, or 100% identical to SEQ ID NO: 140 or 174; (vi) a VH3 comprising an amino acid sequence that is at least 65%, at least 70%, at least 75%, at least 80%, at least 85%, at least 90%, at least 95%, at least 99%, or 100% identical to SEQ ID NO: 144 or 178; (vii) a VL4 comprising an amino acid sequence that is at least 65%, at least 70%, at least 75%, at least 80%, at least 85%, at least 90%, at least 95%, at least 99%, or 100% identical to SEQ ID NO: 140 or 174; and (viii) a VH4 comprising an amino acid sequence that is at least 65%, at least 70%, at least 75%, at least 80%, at least 85%, at least 90%, at least 95%, at least 99%, or 100% identical to SEQ ID NO: 144 or 178.

In some aspects, VL1 comprises an amino acid sequence at least 65%, at least 70%, at least 75%, at least 80%, at least 85%, at least 90%, at least 95%, at least 99%, or 100% identical to SEQ ID NO: 4; and VH1 comprises an amino acid sequence at least 65%, at least 70%, at least 75%, at least 80%, at least 85%, at least 90%, at least 95%, at least 99%, or 100% identical to SEQ ID NO: 8.

In some aspects, VL1 comprises an amino acid sequence at least 65%, at least 70%, at least 75%, at least 80%, at least 85%, at least 90%, at least 95%, at least 99%, or 100% identical to SEQ ID NO: 11; and VH1 comprises an amino acid sequence at least 65%, at least 70%, at least 75%, at least 80%, at least 85%, at least 90%, at least 95%, at least 99%, or 100% identical to SEQ ID NO: 14.

In some aspects, VL1 comprises an amino acid sequence at least 65%, at least 70%, at least 75%, at least 80%, at least 85%, at least 90%, at least 95%, at least 99%, or 100% identical to SEQ ID NO: 17; and VH1 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, VL1 comprises an amino acid sequence at least 65%, at least 70%, at least 75%, at least 80%, at least 85%, at least 90%, at least 95%, at least 99%, or 100% identical to SEQ ID NO: 25; and VH1 comprises an amino acid sequence at least 65%, at least 70%, at least 75%, at least 80%, at least 85%, at least 90%, at least 95%, at least 99%, or 100% identical to SEQ ID NO: 29.

In some aspects, VL1 comprises an amino acid sequence at least 65%, at least 70%, at least 75%, at least 80%, at least 85%, at least 90%, at least 95%, at least 99%, or 100% identical to SEQ ID NO: 33 or 39; and VH1 comprises an amino acid sequence at least 65%, at least 70%, at least 75%, at least 80%, at least 85%, at least 90%, at least 95%, at least 99%, or 100% identical to SEQ ID NO: 37.

In some aspects, VL1 comprises an amino acid sequence at least 65%, at least 70%, at least 75%, at least 80%, at least 85%, at least 90%, at least 95%, at least 99%, or 100% identical to SEQ ID NO: 67; and VH1 comprises an amino acid sequence at least 65%, at least 70%, at least 75%, at least 80%, at least 85%, at least 90%, at least 95%, at least 99%, or 100% identical to SEQ ID NO: 71 or 626.

In some aspects, VL1 comprises an amino acid sequence at least 65%, at least 70%, at least 75%, at least 80%, at least 85%, at least 90%, at least 95%, at least 99%, or 100% identical to SEQ ID NO: 74; and VH1 comprises an amino acid sequence at least 65%, at least 70%, at least 75%, at least 80%, at least 85%, at least 90%, at least 95%, at least 99%, or 100% identical to SEQ ID NO: 78.

In some aspects, VL1 comprises an amino acid sequence at least 65%, at least 70%, at least 75%, at least 80%, at least 85%, at least 90%, at least 95%, at least 99%, or 100% identical to SEQ ID NO: 147; and VH1 comprises an amino acid sequence at least 65%, at least 70%, at least 75%, at least 80%, at least 85%, at least 90%, at least 95%, at least 99%, or 100% identical to SEQ ID NO: 151.

In some aspects, VL1 comprises an amino acid sequence at least 65%, at least 70%, at least 75%, at least 80%, at least 85%, at least 90%, at least 95%, at least 99%, or 100% identical to SEQ ID NO: 154; and VH1 comprises an amino acid sequence at least 65%, at least 70%, at least 75%, at least 80%, at least 85%, at least 90%, at least 95%, at least 99%, or 100% identical to SEQ ID NO: 158.

In some aspects, VL1 comprises an amino acid sequence at least 65%, at least 70%, at least 75%, at least 80%, at least 85%, at least 90%, at least 95%, at least 99%, or 100% identical to SEQ ID NO: 140; and VH1 comprises an amino acid sequence at least 65%, at least 70%, at least 75%, at least 80%, at least 85%, at least 90%, at least 95%, at least 99%, or 100% identical to SEQ ID NO: 144.

In some aspects, VL1 comprises an amino acid sequence at least 65%, at least 70%, at least 75%, at least 80%, at least 85%, at least 90%, at least 95%, at least 99%, or 100% identical to SEQ ID NO: 174; and VH1 comprises an amino acid sequence at least 65%, at least 70%, at least 75%, at least 80%, at least 85%, at least 90%, at least 95%, at least 99%, or 100% identical to SEQ ID NO: 178.

In some aspects, VL2 comprises an amino acid sequence at least 65%, at least 70%, at least 75%, at least 80%, at least 85%, at least 90%, at least 95%, at least 99%, or 100% identical to SEQ ID NO: 4; and VH2 comprises an amino acid sequence at least 65%, at least 70%, at least 75%, at least 80%, at least 85%, at least 90%, at least 95%, at least 99%, or 100% identical to SEQ ID NO: 8.

In some aspects, VL2 comprises an amino acid sequence at least 65%, at least 70%, at least 75%, at least 80%, at least 85%, at least 90%, at least 95%, at least 99%, or 100% identical to SEQ ID NO: 11; and VH2 comprises an amino acid sequence at least 65%, at least 70%, at least 75%, at least 80%, at least 85%, at least 90%, at least 95%, at least 99%, or 100% identical to SEQ ID NO: 14.

In some aspects, VL2 comprises an amino acid sequence at least 65%, at least 70%, at least 75%, at least 80%, at least 85%, at least 90%, at least 95%, at least 99%, or 100% identical to SEQ ID NO: 17; and VH2 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, VL2 comprises an amino acid sequence at least 65%, at least 70%, at least 75%, at least 80%, at least 85%, at least 90%, at least 95%, at least 99%, or 100% identical to SEQ ID NO: 25; and VH2 comprises an amino acid sequence at least 65%, at least 70%, at least 75%, at least 80%, at least 85%, at least 90%, at least 95%, at least 99%, or 100% identical to SEQ ID NO: 29.

In some aspects, VL2 comprises an amino acid sequence at least 65%, at least 70%, at least 75%, at least 80%, at least 85%, at least 90%, at least 95%, at least 99%, or 100% identical to SEQ ID NO: 33 or 39; and VH2 comprises an amino acid sequence at least 65%, at least 70%, at least 75%, at least 80%, at least 85%, at least 90%, at least 95%, at least 99%, or 100% identical to SEQ ID NO: 37.

In some aspects, VL2 comprises an amino acid sequence at least 65%, at least 70%, at least 75%, at least 80%, at least 85%, at least 90%, at least 95%, at least 99%, or 100% identical to SEQ ID NO: 67; and VH2 comprises an amino acid sequence at least 65%, at least 70%, at least 75%, at least 80%, at least 85%, at least 90%, at least 95%, at least 99%, or 100% identical to SEQ ID NO: 71 or 626.

In some aspects, VL2 comprises an amino acid sequence at least 65%, at least 70%, at least 75%, at least 80%, at least 85%, at least 90%, at least 95%, at least 99%, or 100% identical to SEQ ID NO: 74; and VH2 comprises an amino acid sequence at least 65%, at least 70%, at least 75%, at least 80%, at least 85%, at least 90%, at least 95%, at least 99%, or 100% identical to SEQ ID NO: 78.

In some aspects, VL2 comprises an amino acid sequence at least 65%, at least 70%, at least 75%, at least 80%, at least 85%, at least 90%, at least 95%, at least 99%, or 100% identical to SEQ ID NO: 147; and VH2 comprises an amino acid sequence at least 65%, at least 70%, at least 75%, at least 80%, at least 85%, at least 90%, at least 95%, at least 99%, or 100% identical to SEQ ID NO: 151.

In some aspects, VL2 comprises an amino acid sequence at least 65%, at least 70%, at least 75%, at least 80%, at least 85%, at least 90%, at least 95%, at least 99%, or 100% identical to SEQ ID NO: 154; and VH2 comprises an amino acid sequence at least 65%, at least 70%, at least 75%, at least 80%, at least 85%, at least 90%, at least 95%, at least 99%, or 100% identical to SEQ ID NO: 158.

In some aspects, VL2 comprises an amino acid sequence at least 65%, at least 70%, at least 75%, at least 80%, at least 85%, at least 90%, at least 95%, at least 99%, or 100% identical to SEQ ID NO: 140; and VH2 comprises an amino acid sequence at least 65%, at least 70%, at least 75%, at least 80%, at least 85%, at least 90%, at least 95%, at least 99%, or 100% identical to SEQ ID NO: 144.

In some aspects, VL2 comprises an amino acid sequence at least 65%, at least 70%, at least 75%, at least 80%, at least 85%, at least 90%, at least 95%, at least 99%, or 100% identical to SEQ ID NO: 174; and VH2 comprises an amino acid sequence at least 65%, at least 70%, at least 75%, at least 80%, at least 85%, at least 90%, at least 95%, at least 99%, or 100% identical to SEQ ID NO: 178.

In some aspects, VL3 comprises an amino acid sequence at least 65%, at least 70%, at least 75%, at least 80%, at least 85%, at least 90%, at least 95%, at least 99%, or 100% identical to SEQ ID NO: 4; and VH3 comprises an amino acid sequence at least 65%, at least 70%, at least 75%, at least 80%, at least 85%, at least 90%, at least 95%, at least 99%, or 100% identical to SEQ ID NO: 8.

In some aspects, VL3 comprises an amino acid sequence at least 65%, at least 70%, at least 75%, at least 80%, at least 85%, at least 90%, at least 95%, at least 99%, or 100% identical to SEQ ID NO: 11; and VH3 comprises an amino acid sequence at least 65%, at least 70%, at least 75%, at least 80%, at least 85%, at least 90%, at least 95%, at least 99%, or 100% identical to SEQ ID NO: 14.

In some aspects, VL3 comprises an amino acid sequence at least 65%, at least 70%, at least 75%, at least 80%, at least 85%, at least 90%, at least 95%, at least 99%, or 100% identical to SEQ ID NO: 17; and VH3 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, VL3 comprises an amino acid sequence at least 65%, at least 70%, at least 75%, at least 80%, at least 85%, at least 90%, at least 95%, at least 99%, or 100% identical to SEQ ID NO: 25; and VH3 comprises an amino acid sequence at least 65%, at least 70%, at least 75%, at least 80%, at least 85%, at least 90%, at least 95%, at least 99%, or 100% identical to SEQ ID NO: 29.

In some aspects, VL3 comprises an amino acid sequence at least 65%, at least 70%, at least 75%, at least 80%, at least 85%, at least 90%, at least 95%, at least 99%, or 100% identical to SEQ ID NO: 33 or 39; and VH3 comprises an amino acid sequence at least 65%, at least 70%, at least 75%, at least 80%, at least 85%, at least 90%, at least 95%, at least 99%, or 100% identical to SEQ ID NO: 37.

In some aspects, VL3 comprises an amino acid sequence at least 65%, at least 70%, at least 75%, at least 80%, at least 85%, at least 90%, at least 95%, at least 99%, or 100% identical to SEQ ID NO: 67; and VH3 comprises an amino acid sequence at least 65%, at least 70%, at least 75%, at least 80%, at least 85%, at least 90%, at least 95%, at least 99%, or 100% identical to SEQ ID NO: 71 or 626.

In some aspects, VL3 comprises an amino acid sequence at least 65%, at least 70%, at least 75%, at least 80%, at least 85%, at least 90%, at least 95%, at least 99%, or 100% identical to SEQ ID NO: 74; and VH3 comprises an amino acid sequence at least 65%, at least 70%, at least 75%, at least 80%, at least 85%, at least 90%, at least 95%, at least 99%, or 100% identical to SEQ ID NO: 78.

In some aspects, VL3 comprises an amino acid sequence at least 65%, at least 70%, at least 75%, at least 80%, at least 85%, at least 90%, at least 95%, at least 99%, or 100% identical to SEQ ID NO: 147; and VH3 comprises an amino acid sequence at least 65%, at least 70%, at least 75%, at least 80%, at least 85%, at least 90%, at least 95%, at least 99%, or 100% identical to SEQ ID NO: 151.

In some aspects, VL3 comprises an amino acid sequence at least 65%, at least 70%, at least 75%, at least 80%, at least 85%, at least 90%, at least 95%, at least 99%, or 100% identical to SEQ ID NO: 154; and VH3 comprises an amino acid sequence at least 65%, at least 70%, at least 75%, at least 80%, at least 85%, at least 90%, at least 95%, at least 99%, or 100% identical to SEQ ID NO: 158.

In some aspects, VL3 comprises an amino acid sequence at least 65%, at least 70%, at least 75%, at least 80%, at least 85%, at least 90%, at least 95%, at least 99%, or 100% identical to SEQ ID NO: 140; and VH3 comprises an amino acid sequence at least 65%, at least 70%, at least 75%, at least 80%, at least 85%, at least 90%, at least 95%, at least 99%, or 100% identical to SEQ ID NO: 144.

In some aspects, VL3 comprises an amino acid sequence at least 65%, at least 70%, at least 75%, at least 80%, at least 85%, at least 90%, at least 95%, at least 99%, or 100% identical to SEQ ID NO: 174; and VH3 comprises an amino acid sequence at least 65%, at least 70%, at least 75%, at least 80%, at least 85%, at least 90%, at least 95%, at least 99%, or 100% identical to SEQ ID NO: 178.

In some aspects, VL4 comprises an amino acid sequence at least 65%, at least 70%, at least 75%, at least 80%, at least 85%, at least 90%, at least 95%, at least 99%, or 100% identical to SEQ ID NO: 4; and VH4 comprises an amino acid sequence at least 65%, at least 70%, at least 75%, at least 80%, at least 85%, at least 90%, at least 95%, at least 99%, or 100% identical to SEQ ID NO: 8.

In some aspects, VL4 comprises an amino acid sequence at least 65%, at least 70%, at least 75%, at least 80%, at least 85%, at least 90%, at least 95%, at least 99%, or 100% identical to SEQ ID NO: 11; and VH4 comprises an amino acid sequence at least 65%, at least 70%, at least 75%, at least 80%, at least 85%, at least 90%, at least 95%, at least 99%, or 100% identical to SEQ ID NO: 14.

In some aspects, VL4 comprises an amino acid sequence at least 65%, at least 70%, at least 75%, at least 80%, at least 85%, at least 90%, at least 95%, at least 99%, or 100% identical to SEQ ID NO: 17; and VH4 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, VL4 comprises an amino acid sequence at least 65%, at least 70%, at least 75%, at least 80%, at least 85%, at least 90%, at least 95%, at least 99%, or 100% identical to SEQ ID NO: 25; and VH4 comprises an amino acid sequence at least 65%, at least 70%, at least 75%, at least 80%, at least 85%, at least 90%, at least 95%, at least 99%, or 100% identical to SEQ ID NO: 29.

In some aspects, VL4 comprises an amino acid sequence at least 65%, at least 70%, at least 75%, at least 80%, at least 85%, at least 90%, at least 95%, at least 99%, or 100% identical to SEQ ID NO: 33 or 39; and VH4 comprises an amino acid sequence at least 65%, at least 70%, at least 75%, at least 80%, at least 85%, at least 90%, at least 95%, at least 99%, or 100% identical to SEQ ID NO: 37.

In some aspects, VL4 comprises an amino acid sequence at least 65%, at least 70%, at least 75%, at least 80%, at least 85%, at least 90%, at least 95%, at least 99%, or 100% identical to SEQ ID NO: 67; and VH4 comprises an amino acid sequence at least 65%, at least 70%, at least 75%, at least 80%, at least 85%, at least 90%, at least 95%, at least 99%, or 100% identical to SEQ ID NO: 71 or 626.

In some aspects, VL4 comprises an amino acid sequence at least 65%, at least 70%, at least 75%, at least 80%, at least 85%, at least 90%, at least 95%, at least 99%, or 100% identical to SEQ ID NO: 74; and VH4 comprises an amino acid sequence at least 65%, at least 70%, at least 75%, at least 80%, at least 85%, at least 90%, at least 95%, at least 99%, or 100% identical to SEQ ID NO: 78.

In some aspects, VL4 comprises an amino acid sequence at least 65%, at least 70%, at least 75%, at least 80%, at least 85%, at least 90%, at least 95%, at least 99%, or 100% identical to SEQ ID NO: 147; and VH4 comprises an amino acid sequence at least 65%, at least 70%, at least 75%, at least 80%, at least 85%, at least 90%, at least 95%, at least 99%, or 100% identical to SEQ ID NO: 151.

In some aspects, VL4 comprises an amino acid sequence at least 65%, at least 70%, at least 75%, at least 80%, at least 85%, at least 90%, at least 95%, at least 99%, or 100% identical to SEQ ID NO: 154; and VH4 comprises an amino acid sequence at least 65%, at least 70%, at least 75%, at least 80%, at least 85%, at least 90%, at least 95%, at least 99%, or 100% identical to SEQ ID NO: 158.

In some aspects, VL4 comprises an amino acid sequence at least 65%, at least 70%, at least 75%, at least 80%, at least 85%, at least 90%, at least 95%, at least 99%, or 100% identical to SEQ ID NO: 140; and VH4 comprises an amino acid sequence at least 65%, at least 70%, at least 75%, at least 80%, at least 85%, at least 90%, at least 95%, at least 99%, or 100% identical to SEQ ID NO: 144.

In some aspects, VL4 comprises an amino acid sequence at least 65%, at least 70%, at least 75%, at least 80%, at least 85%, at least 90%, at least 95%, at least 99%, or 100% identical to SEQ ID NO: 174; and VH4 comprises an amino acid sequence at least 65%, at least 70%, at least 75%, at least 80%, at least 85%, at least 90%, at least 95%, at least 99%, or 100% identical to SEQ ID NO: 178.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, an antigen binding polypeptide complex provided herein comprises a first polypeptide, and a second polypeptide; wherein the first polypeptide has a structure represented by VL1-L1-VL2-L2-CH1 or VL2-L1-VL1-L2-CH1; and wherein the second polypeptide has a structure represented by VH1-L3-VH2-L4-CL or VH2-L3-VH1-L4-CL.

In some aspects, an antigen binding polypeptide complex provided herein comprises a first polypeptide, and a second polypeptide; wherein the first polypeptide has a structure represented by VL1-VL2-CH1-CH2-CH3 or VL2-VL1-CH1-CH2-CH3; and wherein the second polypeptide has a structure represented by VH1-VH2-CL-CH2-CH3 or VH2-VH1-CL-CH2-CH3.

In some aspects, an antigen binding polypeptide complex provided herein comprises a first polypeptide, and a second polypeptide; wherein the first polypeptide has a structure represented by VL1-L1-VL2-L2-CH1-CH2-CH3 or VL2-L1-VL1-L2-CH1-CH2-CH3; and wherein the second polypeptide has a structure represented by VH1-L3-VH2-L4-CL-CH2-CH3 or VH2-L3-VH1-L4-CL-CH2-CH3.

In some aspects, VL1 is a first immunoglobulin light chain variable region that specifically binds to a SARS-CoV-2 protein (e.g., a SARS-CoV-2 spike protein); VL2 is a second immunoglobulin light chain variable region that specifically binds to a SARS-CoV-2 protein (e.g., a SARS-CoV-2 spike protein); VH1 is a first immunoglobulin heavy chain variable region that specifically binds to a SARS-CoV-2 protein (e.g., a SARS-CoV-2 spike protein); and VH2 is a second immunoglobulin heavy chain variable region that specifically binds to a SARS-CoV-2 protein (e.g., a SARS-CoV-2 spike protein); CH1 is an immunoglobulin heavy chain constant region 1; CL is an immunoglobulin light chain constant region; and L1-L4 are optional amino acid linkers. In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the VL1 and the VL2 each comprise a CDR1, a CDR2, and a CDR3 that are the same. In some aspects, the VL1 and the VL2 each comprise a CDR1, a CDR2, and a CDR3 that are different.

In some aspects, the VL1 and the VL2 each comprise an amino acid sequence, wherein the amino acid sequence comprised in VL1 and the amino acid sequence comprised in VL2 are the same. In some aspects, the VL1 and the VL2 each comprise an amino acid sequence, wherein the amino acid sequence comprised in VL1 and the amino acid sequence comprised in VL2 are different.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, at least one of VL1 or VL2 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprised in the antigen binding polypeptide complexes provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1 or VH2 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the second antigen binding polypeptide comprised in the antigen binding polypeptide complexes disclosed herein, comprises one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the first antigen binding polypeptide comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NO: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, an antigen binding polypeptide complex provided herein comprises a first polypeptide, and a second polypeptide; wherein the first polypeptide has a structure represented by VL1-VH1-CL or VH1-VL1-CL; and wherein the second polypeptide has a structure represented by VL2-VH2-CH1 or VH2-VL2-CH1.

In some aspects, an antigen binding polypeptide complex provided herein comprises a first polypeptide, and a second polypeptide; wherein the first polypeptide has a structure represented by VL1-L1-VH1-L2-CL or VH1-L1-VL1-L2-CL; and wherein the second polypeptide has a structure represented by VL2-L3-VH2-L4-CH1 or VH2-L3-VL2-L4-CH1.

In some aspects, an antigen binding polypeptide complex provided herein comprises a first polypeptide, and a second polypeptide; wherein the first polypeptide has a structure represented by VL1-VH1-CL-CH2-CH3 or VH1-VL1-CL-CH2-CH3; and wherein the second polypeptide has a structure represented by VL2-VH2-CH1-CH2-CH3 or VH2-VL2-CH1-CH2-CH3.

In some aspects, an antigen binding polypeptide complex provided herein comprises a first polypeptide, and a second polypeptide; wherein the first polypeptide has a structure represented by VL1-L1-VH1-L2-CL-CH2-CH3 or VH1-L1-VL1-L2-CL-CH2-CH3; and wherein the second polypeptide has a structure represented by VL2-L3-VH2-L4-CH1-CH2-CH3 or VH2-L3-VL2-L4-CH1-CH2-CH3.

In some aspects, VL1 is a first immunoglobulin light chain variable region that specifically binds to a SARS-CoV-2 protein (e.g., a SARS-CoV-2 spike protein); VL2 is a second immunoglobulin light chain variable region that specifically binds to a SARS-CoV-2 protein (e.g., a SARS-CoV-2 spike protein); VH1 is a first immunoglobulin heavy chain variable region that specifically binds to a SARS-CoV-2 protein (e.g., a SARS-CoV-2 spike protein); VH2 is a second immunoglobulin heavy chain variable region that specifically binds to a SARS-CoV-2 protein (e.g., a SARS-CoV-2 spike protein); CH1 is an immunoglobulin heavy chain constant region 1; CL is an immunoglobulin light chain constant region; and L1-L4 are optional amino acid linkers. In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the VL1 and the VL2 each comprise a CDR1, a CDR2, and a CDR3 that are the same. In some aspects, the VL1 and the VL2 each comprise a CDR1, a CDR2, and a CDR3 that are different.

In some aspects, the VL1 and the VL2 each comprise an amino acid sequence, wherein the amino acid sequence comprised in VL1 and the amino acid sequence comprised in VL2 are the same. In some aspects, the VL1 and the VL2 each comprise an amino acid sequence, wherein the amino acid sequence comprised in VL1 and the amino acid sequence comprised in VL2 are different.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325 326, 764, and 766.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, VL1 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VH1 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325 326, 764, and 766.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10.

In some aspects, VL2 comprises a CDR1 having an amino acid sequence at least 65%, at least 70%, at least 75%, at least 80%, at least 85%, at least 90%, at least 95%, at least 99%, or 100% identical to SEQ ID NO: 72, a CDR2 having an amino acid sequence at least 65%, at least 70%, at least 75%, at least 80%, at least 85%, at least 90%, at least 95%, at least 99%, or 100% identical to amino acid sequence DNT, and a CDR3 having an amino acid sequence at least 65%, at least 70%, at least 75%, at least 80%, at least 85%, at least 90%, at least 95%, at least 99%, or 100% identical to SEQ ID NO: 73.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059.

In some aspects, VH2 comprises a CDR1 having an amino acid sequence at least 65%, at least 70%, at least 75%, at least 80%, at least 85%, at least 90%, at least 95%, at least 99%, or 100% identical to SEQ ID NO: 75, a CDR2 having an amino acid sequence at least 65%, at least 70%, at least 75%, at least 80%, at least 85%, at least 90%, at least 95%, at least 99%, or 100% identical to SEQ ID NO: 76, and a CDR3 having an amino acid sequence at least 65%, at least 70%, at least 75%, at least 80%, at least 85%, at least 90%, at least 95%, at least 99%, or 100% identical to SEQ ID NO: 77.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, at least one of VL1 or VL2 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprised in the antigen binding polypeptide complexes provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1 or VH2 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884. In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the first antigen binding polypeptide comprised in the antigen binding polypeptide complexes disclosed herein, comprises one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the second antigen binding polypeptide comprised in the antigen binding polypeptide complexes disclosed herein, comprises one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOS: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, an antigen binding polypeptide complex provided herein comprises a first polypeptide, and a second polypeptide; wherein the first polypeptide has a structure represented by VL1-VL2-VL3-CH1, VL1-VL3-VL2-CH1, VL2-VL1-VL3-CH1, VL2-VL3-VL1-CH1, VL3-VL1-VL2-CH1, or VL3-VL2-VL1-CH1; and wherein the second polypeptide has a structure represented by VH1-VH2-VH3-CL, VH1-VH3-VH2-CL, VH2-VH1-VH3-CL, VH2-VH3-VH1-CL, VH3-VH1-VH2-CL, or VH3-VH2-VH1-CL.

In some aspects, an antigen binding polypeptide complex provided herein comprises a first polypeptide, and a second polypeptide; wherein the first polypeptide has a structure represented by VL1-L1-VL2-L2-VL3-L3-CH1, VL1-L1-VL3-L2-VL2-L3-CH1, VL2-L1-VL1-L2-VL3-L3-CH1, VL2-L1-VL3-L2-VL1-L3-CH1, VL3-L1-VL1-L2-VL2-L3-CH1, or VL3-L1-VL2-L2-VL1-L3-CH1; and wherein the second polypeptide has a structure represented by VH1-L4-VH2-L5-VH3-L6-CL, VH1-L4-VH3-L5-VH2-L6-CL, VH2-L4-VH1-L5-VH3-L6-CL, VH2-L4-VH3-L5-VH1-L6-CL, VH3-L4-VH1-L5-VH2-L6-CL, or VH3-L4-VH2-L5-VH1-L6-CL.

In some aspects, an antigen binding polypeptide complex provided herein comprises a first polypeptide, and a second polypeptide; wherein the first polypeptide has a structure represented by VL1-VL2-VL3-CH1-CH2-CH3, VL1-VL3-VL2-CH1-CH2-CH3, VL2-VL1-VL3-CH1-CH2-CH3, VL2-VL3-VL1-CH1-CH2-CH3, VL3-VL1-VL2-CH1-CH2-CH3, or VL3-VL2-VL1-CH1-CH2-CH3; and wherein the second polypeptide has a structure represented by VH1-VH2-VH3-CL-CH2-CH3, VH1-VH3-VH2-CL-CH2-CH3, VH2-VH1-VH3-CL-CH2-CH3, VH2-VH3-VH1-CL-CH2-CH3, VH3-VH1-VH2-CL-CH2-CH3, or VH3-VH2-VH1-CL-CH2-CH3.

In some aspects, an antigen binding polypeptide complex provided herein comprises a first polypeptide, and a second polypeptide; wherein the first polypeptide has a structure represented by VL1-L1-VL2-L2-VL3-L3-CH1-CH2-CH3, VL1-L1-VL3-L2-VL2-L3-CH1-CH2-CH3, VL2-L1-VL1- L2-VL3-L3-CH1-CH2-CH3, VL2-L1-VL3-L2-VL1-L3-CH1-CH2-CH3, VL3-L1-VL1-L2-VL2-L3-CH1- CH2-CH3, or VL3-L1-VL2-L2-VL1-L3-CH1-CH2-CH3; and wherein the second polypeptide has a structure represented by VH1-L1-VH2-L2-VH3-L3-CL-CH2-CH3, VH1-L1-VH3-L2-VH2-L3-CL-CH2-CH3, VH2-L1-VH1-L2-VH3-L3-CL-CH2-CH3, VH2-L1-VH3-L2-VH1-L3-CL-CH2-CH3, VH3-L1-VH1-L2-VH2-L3-CL-CH2-CH3, or VH3-L1-VH2-L2-VH1-L3-CL-CH2-CH3.

In some aspects, VL1 is a first immunoglobulin light chain variable region that specifically binds to a SARS-CoV-2 protein (e.g., a SARS-CoV-2 spike protein); VL2 is a second immunoglobulin light chain variable region that specifically binds to a SARS-CoV-2 protein (e.g., a SARS-CoV-2 spike protein); VL3 is a third immunoglobulin light chain variable region that specifically binds to a SARS-CoV-2 protein (e.g., a SARS-CoV-2 spike protein); VH1 is a first immunoglobulin heavy chain variable region that specifically binds to a SARS-CoV-2 protein (e.g., a SARS-CoV-2 spike protein); VH2 is a second immunoglobulin heavy chain variable region that specifically binds to a SARS-CoV-2 protein (e.g., a SARS-CoV-2 spike protein); VH3 is a third immunoglobulin heavy chain variable region that specifically binds to a SARS-CoV-2 protein (e.g., a SARS-CoV-2 spike protein); CH1 is an immunoglobulin heavy chain constant region 1; CL is an immunoglobulin light chain constant region; and L1-L6 are optional amino acid linkers. In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the VL1 and the VL2 each comprise a CDR1, a CDR2, and a CDR3 that are the same. In some aspects, the VL1 and the VL2 each comprise a CDR1, a CDR2, and a CDR3 that are different. In some aspects, the VL1 and the VL3 each comprise a CDR1, a CDR2, and a CDR3 that are the same. In some aspects, the VL1 and the VL3 each comprise a CDR1, a CDR2, and a CDR3 that are different. In some aspects, the VL2 and the VL3 each comprise a CDR1, a CDR2, and a CDR3 that are the same. In some aspects, the VL2 and the VL3 each comprise a CDR1, a CDR2, and a CDR3 that are different.

In some aspects, the VL1 and the VL2 each comprise an amino acid sequence, wherein the amino acid sequence comprised in VL1 and the amino acid sequence comprised in VL2 are the same. In some aspects, the VL1 and the VL2 each comprise an amino acid sequence, wherein the amino acid sequence comprised in VL1 and the amino acid sequence comprised in VL2 are different. In some aspects, the VL1 and the VL3 each comprise an amino acid sequence, wherein the amino acid sequence comprised in VL1 and the amino acid sequence comprised in VL3 are the same. In some aspects, the VL1 and the VL3 each comprise an amino acid sequence, wherein the amino acid sequence comprised in VL1 and the amino acid sequence comprised in VL3 are different. In some aspects, the VL2 and the VL3 each comprise an amino acid sequence, wherein the amino acid sequence comprised in VL2 and the amino acid sequence comprised in VL3 are the same. In some aspects, the VL2 and the VL3 each comprise an amino acid sequence, wherein the amino acid sequence comprised in VL2 and the amino acid sequence comprised in VL3 are different.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL1 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL1 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL1 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL1 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH1 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH1 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH1 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH1 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL2 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL2 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL2 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL2 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH2 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH2 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH2 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH2 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, VL3 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065.

In some aspects, VL3 comprises a CDR1 having an amino acid sequence at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065.

In some aspects, VL3 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT.

In some aspects, VL3 comprises a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT.

In some aspects, VL3 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, VL3 comprises a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, VL3 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, VL3 comprises an amino acid sequence at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, VH3 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067.

In some aspects, VH3 comprises a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067.

In some aspects, VH3 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068.

In some aspects, VH3 comprises a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068. In some aspects, VH3 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, VH3 comprises a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 150, 157, 327, 332, 631, 765, 767, 143, 857, 177, 793, 1049, 1054, 1059, 1064, and 1069.

In some aspects, VH3 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, VH3 comprises an amino acid sequence at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, at least one of VL1, VL2, or VL3 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1, VH2, or VH3 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1, VL2, or VL3 comprised in the antigen binding polypeptide complexes provided herein comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1, VH2, or VH3 comprised in the antigen binding polypeptide complexes provided herein comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1, VL2, or VL3 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1, VH2, or VH3 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1, VL2, or VL3 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1, VH2, or VH3 comprised in the antigen binding polypeptide complexes provided herein comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; a VL3 comprising (xiii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (xiv) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (xv) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and a VH3 comprising (xvi) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xvii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; (xviii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (v) a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (vi) a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; a VH1 comprising (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; a VL2 comprising (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; a VH2 comprising (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; a VL3 comprising (xiii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (xiv) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (xv) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and a VH3 comprising (xvi) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xvii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xviii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the antigen binding polypeptide complexes provided herein comprise (i) a VL1 comprising an amino acid sequence that is at least 65%, at least 70%, at least 75%, at least 80%, at least 85%, at least 90%, at least 95%, at least 99%, or 100% identical to SEQ ID NO: 147; (ii) a VH1 comprising an amino acid sequence that is at least 65%, at least 70%, at least 75%, at least 80%, at least 85%, at least 90%, at least 95%, at least 99%, or 100% identical to SEQ ID NO: 151; (iii) a VL2 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (iv) a VH2 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (v) a VL3 comprising an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (vi) a VH3 comprising an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the second antigen binding polypeptide comprised in the antigen binding polypeptide complexes disclosed herein, comprises one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the first antigen binding polypeptide comprised in the antigen binding polypeptide complexes disclosed herein, comprises one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to SEQ ID Ns: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the first polypeptide comprises a structure represented by VL1-CL-VL2-VH2-VH1-CH1, VL1-L1-CL-L2-VL2-L3-VH2-L4-VH1-L5-CH1, VL1-CL-L1-VL2-L2-VH2-L3-VH1-CH1, VL1-CL-VL2-VH2-VH1-CH1-CH2-CH3, VL1-L1-CL-L2-VL2-L3-VH2-L4-VH1-L5-CH1-CH2-CH3, or VL1-CL-L1-VL2-L2-VH2-L3-VH1-CH1-CH2-CH3, and the second polypeptide comprises a structure represented by VL3-CL-VL4-VH4-VH3-CH1, VL3-L6-CL-L7-VL4-L8-VH4-L9-VH3-L10-CH1, VL3-CL-L4-VL4-L5-VH4-L6-VH3-CH1, VL3-CL-VL4-VH4-VH3-CH1-CH2-CH3, VL3-L6-CL-L7-VL4-L8-VH4-L9-VH3-L10-CH1-CH2-CH3, or VL3-CL-L4-VL4-L5-VH4-L6-VH3-CH1-CH2-CH3, wherein:
(a) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059, or wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 17, VH1 and VH3 each comprise an amino acid sequence at least 65%, at least 70%, at least 75%, at least 80%, at least 85%, at least 90%, at least 95%, at least 99%, or 100% identical to SEQ ID NO: 21 or 22, VL2 and VL4 each comprise an amino acid sequence at least 65%, at least 70%, at least 75%, at least 80%, at least 85%, at least 90%, at least 95%, at least 99%, or 100% identical to SEQ ID NO: 11, and VH2 and VH4 each comprise an amino acid sequence at least 65%, at least 70%, at least 75%, at least 80%, at least 85%, at least 90%, at least 95%, at least 99%, or 100% identical to SEQ ID NO: 14; or
(b) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 17, and VH2 and VH4 each comprise an amino acid sequence at least 65%, at least 70%, at least 75%, at least 80%, at least 85%, at least 90%, at least 95%, at least 99%, or 100% identical to SEQ ID NO: 21 or 22; or
(c) VL1 comprises the same light chain variable region as VL4, VH1 comprises the same heavy chain variable region as VH4, VL2 comprises the same light chain variable region as VL3, VH2 comprises the same heavy chain variable region as VH3, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
wherein:
VL1 and VL4 each comprise an amino acid sequence at least 65%, at least 70%, at least 75%, at least 80%, at least 85%, at least 90%, at least 95%, at least 99%, or 100% identical SEQ ID NO: 17, VH1 and VH4 each comprise an amino acid sequence at least 65%, at least 70%, at least 75%, at least 80%, at least 85%, at least 90%, at least 95%, at least 99%, or 100% identical to SEQ ID NO: 21 or 22, VL2 and VL3 each comprise an amino acid sequence at least 65%, at least 70%, at least 75%, at least 80%, at least 85%, at least 90%, at least 95%, at least 99%, or 100% identical to SEQ ID NO: 11, and VH2 and VH3 each comprise an amino acid sequence at least 65%, at least 70%, at least 75%, at least 80%, at least 85%, at least 90%, at least 95%, at least 99%, or 100% identical to SEQ ID NO: 14; or
(d) VL1 comprises the same light chain variable region as VL4, VH1 comprises the same heavy chain variable region as VH4, VL2 comprises the same light chain variable region as VL3, VH2 comprises the same heavy chain variable region as VH3, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059, or
wherein:
VL1 and VL4 each comprise an amino acid sequence at least 65%, at least 70%, at least 75%, at least 80%, at least 85%, at least 90%, at least 95%, at least 99%, or 100% identical to SEQ ID NO: 11, VH1 and VH4 each comprise an amino acid sequence at least 65%, at least 70%, at least 75%, at least 80%, at least 85%, at least 90%, at least 95%, at least 99%, or 100% identical to SEQ ID NO: 14, VL2 and VL3 each comprise an amino acid sequence at least 65%, at least 70%, at least 75%, at least 80%, at least 85%, at least 90%, at least 95%, at least 99%, or 100% identical to SEQ ID NO: 17, and VH2 and VH3 each comprise an amino acid sequence at least 65%, at least 70%, at least 75%, at least 80%, at least 85%, at least 90%, at least 95%, at least 99%, or 100% identical to SEQ ID NO: 21 or 22; or
(e) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059, or
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 17, and VH2 and VH4 each comprise an amino acid sequence at least 65%, at least 70%, at least 75%, at least 80%, at least 85%, at least 90%, at least 95%, at least 99%, or 100% identical to SEQ ID NO: 21 or 22; or
(f) VL2 comprises the same light chain variable region as VL3, VH2 comprises the same heavy chain variable region as VH3, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325,
VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
wherein:
VL1 comprises an amino acid sequence at least 65%, at least 70%, at least 75%, at least 80%, at least 85%, at least 90%, at least 95%, at least 99%, or 100% identical to SEQ ID NO: 25, VH1 comprises an amino acid sequence at least 65%, at least 70%, at least 75%, at least 80%, at least 85%, at least 90%, at least 95%, at least 99%, or 100% identical to SEQ ID NO: 29, VL2 and VL3 each comprise an amino acid sequence at least 65%, at least 70%, at least 75%, at least 80%, at least 85%, at least 90%, at least 95%, at least 99%, or 100% identical to SEQ ID NO: 11, VH2 and VH3 each comprise an amino acid sequence at least 65%, at least 70%, at least 75%, at least 80%, at least 85%, at least 90%, at least 95%, at least 99%, or 100% identical to SEQ ID NO: 14, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(g) VL2 comprises the same light chain variable region as VL4, VH2 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325,
VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059, or
wherein:
VL1 comprises an amino acid sequence at least 65%, at least 70%, at least 75%, at least 80%, at least 85%, at least 90%, at least 95%, at least 99%, or 100% identical to SEQ ID NO: 25, VH1 comprises an amino acid sequence at least 65%, at least 70%, at least 75%, at least 80%, at least 85%, at least 90%, at least 95%, at least 99%, or 100% identical to SEQ ID NO: 29, VL2 and VL4 each comprise an amino acid sequence at least 65%, at least 70%, at least 75%, at least 80%, at least 85%, at least 90%, at least 95%, at least 99%, or 100% identical to SEQ ID NO: 11, VH2 and VH4 each comprise an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22; or
(h) VL1 comprises the same light chain variable region as VL3, VH1 comprises the same heavy chain variable region as VH3, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325,
VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332,
VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(i) VL1 comprises the same light chain variable region as VL4, VH1 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325,
VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059, or
wherein:
VL1 and VL4 each comprise an amino acid sequence at least 65%, at least 70%, at least 75%, at least 80%, at least 85%, at least 90%, at least 95%, at least 99%, or 100% identical to SEQ ID NO: 11, VH1 and VH4 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22; or
(j)
VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325,
VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332,
VL2 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3,
VH2 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327, or
VL1 comprises an amino acid sequence at least 65%, at least 70%, at least 75%, at least 80%, at least 85%, at least 90%, at least 95%, at least 99%, or 100% identical to SEQ ID NO: 25, VH1 comprises an amino acid sequence at least 65%, at least 70%, at least 75%, at least 80%, at least 85%, at least 90%, at least 95%, at least 99%, or 100% identical to SEQ ID NO: 29, VL2 comprises an amino acid sequence at least 65%, at least 70%, at least 75%, at least 80%, at least 85%, at least 90%, at least 95%, at least 99%, or 100% identical to SEQ ID NO: 4, VH2 comprises an amino acid sequence at least 65%, at least 70%, at least 75%, at least 80%, at least 85%, at least 90%, at least 95%, at least 99%, or 100% identical to SEQ ID NO: 8, VL3 comprises an amino acid sequence at least 65%, at least 70%, at least 75%, at least 80%, at least 85%, at least 90%, at least 95%, at least 99%, or 100% identical to SEQ ID NO: 17, VH3 comprises an amino acid sequence at least 65%, at least 70%, at least 75%, at least 80%, at least 85%, at least 90%, at least 95%, at least 99%, or 100% identical to SEQ ID NO: 21 or 22, VL4 comprises an amino acid sequence at least 65%, at least 70%, at least 75%, at least 80%, at least 85%, at least 90%, at least 95%, at least 99%, or 100% identical to SEQ ID NO: 33 or 39, and VH4 comprises an amino acid sequence at least 65%, at least 70%, at least 75%, at least 80%, at least 85%, at least 90%, at least 95%, at least 99%, or 100% identical to SEQ ID NO: 37; or
(k)
VL1 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325,
VH1 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332,
VL2 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3,
VH2 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7,
VL3 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326,
VH3 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
VL1 comprises an amino acid sequence at least 65%, at least 70%, at least 75%, at least 80%, at least 85%, at least 90%, at least 95%, at least 99%, or 100% identical to SEQ ID NO: 25, VH1 comprises an amino acid sequence at least 65%, at least 70%, at least 75%, at least 80%, at least 85%, at least 90%, at least 95%, at least 99%, or 100% identical to SEQ ID NO: 29, VL2 comprises an amino acid sequence at least 65%, at least 70%, at least 75%, at least 80%, at least 85%, at least 90%, at least 95%, at least 99%, or 100% identical to SEQ ID NO: 4, VH2 comprises an amino acid sequence at least 65%, at least 70%, at least 75%, at least 80%, at least 85%, at least 90%, at least 95%, at least 99%, or 100% identical to SEQ ID NO: 8, VL3 comprises an amino acid sequence at least 65%, at least 70%, at least 75%, at least 80%, at least 85%, at least 90%, at least 95%, at least 99%, or 100% identical to SEQ ID NO: 33 or 39, VH3 comprises an amino acid sequence at least 65%, at least 70%, at least 75%, at least 80%, at least 85%, at least 90%, at least 95%, at least 99%, or 100% identical to SEQ ID NO: 37, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(l)
VL1 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3,
VH1 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7,
VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325,
VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327, or
VL1 comprises an amino acid sequence at least 65%, at least 70%, at least 75%, at least 80%, at least 85%, at least 90%, at least 95%, at least 99%, or 100% identical to SEQ ID NO: 4, VH1 comprises an amino acid sequence at least 65%, at least 70%, at least 75%, at least 80%, at least 85%, at least 90%, at least 95%, at least 99%, or 100% identical to SEQ ID NO: 8, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 17, VH3 comprises an amino acid sequence at least 65%, at least 70%, at least 75%, at least 80%, at least 85%, at least 90%, at least 95%, at least 99%, or 100% identical to SEQ ID NO: 21 or 22, VL4 comprises an amino acid sequence at least 65%, at least 70%, at least 75%, at least 80%, at least 85%, at least 90%, at least 95%, at least 99%, or 100% identical to SEQ ID NO: 33, or 39, and VH4 comprises an amino acid sequence at least 65%, at least 70%, at least 75%, at least 80%, at least 85%, at least 90%, at least 95%, at least 99%, or 100% identical to SEQ ID NO: 37; or
(m)
VL1 comprises a CDR1 having an amino acid sequence at least 65%, at least 70%, at least 75%, at least 80%, at least 85%, at least 90%, at least 95%, at least 99%, or 100% identical to SEQ ID NO: 1 or 324, a CDR2 having an amino acid sequence at least 65%, at least 70%, at least 75%, at least 80%, at least 85%, at least 90%, at least 95%, at least 99%, or 100% identical to SEQ ID NO: 2 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 3,
VH1 comprises a CDR1 having an amino acid sequence at least 65%, at least 70%, at least 75%, at least 80%, at least 85%, at least 90%, at least 95%, at least 99%, or 100% identical to SEQ ID NO: 5, a CDR2 having an amino acid sequence at least 65%, at least 70%, at least 75%, at least 80%, at least 85%, at least 90%, at least 95%, at least 99%, or 100% identical to SEQ ID NO: 6, and a CDR3 having an amino acid sequence at least 65%, at least 70%, at least 75%, at least 80%, at least 85%, at least 90%, at least 95%, at least 99%, or 100% identical to SEQ ID NO: 7,
VL2 comprises a CDR1 having an amino acid sequence at least 65%, at least 70%, at least 75%, at least 80%, at least 85%, at least 90%, at least 95%, at least 99%, or 100% identical to SEQ ID NO: 23, a CDR2 having an amino acid sequence at least 65%, at least 70%, at least 75%, at least 80%, at least 85%, at least 90%, at least 95%, at least 99%, or 100% identical to amino acid sequence GTN, and a CDR3 having an amino acid sequence at least 65%, at least 70%, at least 75%, at least 80%, at least 85%, at least 90%, at least 95%, at least 99%, or 100% identical to SEQ ID NO: 24 or 325,
VH2 comprises a CDR1 having an amino acid sequence at least 65%, at least 70%, at least 75%, at least 80%, at least 85%, at least 90%, at least 95%, at least 99%, or 100% identical to SEQ ID NO: 26, a CDR2 having an amino acid sequence at least 65%, at least 70%, at least 75%, at least 80%, at least 85%, at least 90%, at least 95%, at least 99%, or 100% identical to SEQ ID NO: 27, and a CDR3 having an amino acid sequence at least 65%, at least 70%, at least 75%, at least 80%, at least 85%, at least 90%, at least 95%, at least 99%, or 100% identical to SEQ ID NO: 28 or 332,
VL3 comprises a CDR1 having an amino acid sequence at least 65%, at least 70%, at least 75%, at least 80%, at least 85%, at least 90%, at least 95%, at least 99%, or 100% identical to SEQ ID NO: 30, a CDR2 having an amino acid sequence at least 65%, at least 70%, at least 75%, at least 80%, at least 85%, at least 90%, at least 95%, at least 99%, or 100% identical to SEQ ID NO: 31 or an amino acid sequence at least 65%, at least 70%, at least 75%, at least 80%, at least 85%, at least 90%, at least 95%, at least 99%, or 100% identical to amino acid sequence SAS, and a CDR3 having an amino acid sequence at least 65%, at least 70%, at least 75%, at least 80%, at least 85%, at least 90%, at least 95%, at least 99%, or 100% identical to SEQ ID NO: 32, 38, or 326,
VH3 comprises a CDR1 having an amino acid sequence at least 65%, at least 70%, at least 75%, at least 80%, at least 85%, at least 90%, at least 95%, at least 99%, or 100% identical to SEQ ID NO: 34, a CDR2 having an amino acid sequence at least 65%, at least 70%, at least 75%, at least 80%, at least 85%, at least 90%, at least 95%, at least 99%, or 100% identical to SEQ ID NO: 35, and a CDR3 having an amino acid sequence at least 65%, at least 70%, at least 75%, at least 80%, at least 85%, at least 90%, at least 95%, at least 99%, or 100% identical to SEQ ID NO: 36, 40, or 327,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
VL1 comprises an amino acid sequence at least 65%, at least 70%, at least 75%, at least 80%, at least 85%, at least 90%, at least 95%, at least 99%, or 100% identical to SEQ ID NO: 4, VH1 comprises an amino acid sequence at least 65%, at least 70%, at least 75%, at least 80%, at least 85%, at least 90%, at least 95%, at least 99%, or 100% identical to SEQ ID NO: 8, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 33 or 39, VH3 comprises an amino acid sequence at least 65%, at least 70%, at least 75%, at least 80%, at least 85%, at least 90%, at least 95%, at least 99%, or 100% identical to SEQ ID NO: 37, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(n)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 67, VH2 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(o)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 67, VH2 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(p)
VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
VL1 comprises an amino acid sequence at least 65%, at least 70%, at least 75%, at least 80%, at least 85%, at least 90%, at least 95%, at least 99%, or 100% identical to SEQ ID NO: 147, VH1 comprises an amino acid sequence at least 65%, at least 70%, at least 75%, at least 80%, at least 85%, at least 90%, at least 95%, at least 99%, or 100% identical to SEQ ID NO: 151, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 67, VH3 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(q)
VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049, or
VL1 comprises an amino acid sequence at least 65%, at least 70%, at least 75%, at least 80%, at least 85%, at least 90%, at least 95%, at least 99%, or 100% identical to SEQ ID NO: 147, VH1 comprises an amino acid sequence at least 65%, at least 70%, at least 75%, at least 80%, at least 85%, at least 90%, at least 95%, at least 99%, or 100% identical to SEQ ID NO: 151, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 67, VH3 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 154, and VH4 comprises an amino acid sequence at least 65%, at least 70%, at least 75%, at least 80%, at least 85%, at least 90%, at least 95%, at least 99%, or 100% identical to SEQ ID NO: 158; or
(r)
VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631, or VL1 comprises an amino acid sequence at least 65%, at least 70%, at least 75%, at least 80%, at least 85%, at least 90%, at least 95%, at least 99%, or 100% identical to SEQ ID NO: 147, VH1 comprises an amino acid sequence at least 65%, at least 70%, at least 75%, at least 80%, at least 85%, at least 90%, at least 95%, at least 99%, or 100% identical to SEQ ID NO: 151, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 17, VH3 comprises an amino acid sequence at least 65%, at least 70%, at least 75%, at least 80%, at least 85%, at least 90%, at least 95%, at least 99%, or 100% identical to SEQ ID NO: 21 or 22, VL4 comprises an amino acid sequence at least 65%, at least 70%, at least 75%, at least 80%, at least 85%, at least 90%, at least 95%, at least 99%, or 100% identical to SEQ ID NO: 67, and VH4 comprises an amino acid sequence at least 65%, at least 70%, at least 75%, at least 80%, at least 85%, at least 90%, at least 95%, at least 99%, or 100% identical to SEQ ID NO: 71 or 626; or(s) VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766,
VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049,
VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631, or
VL1 comprises an amino acid sequence at least 65%, at least 70%, at least 75%, at least 80%, at least 85%, at least 90%, at least 95%, at least 99%, or 100% identical to SEQ ID NO: 147, VH1 comprises an amino acid sequence at least 65%, at least 70%, at least 75%, at least 80%, at least 85%, at least 90%, at least 95%, at least 99%, or 100% identical to SEQ ID NO: 151, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 154, VH3 comprises an amino acid sequence at least 65%, at least 70%, at least 75%, at least 80%, at least 85%, at least 90%, at least 95%, at least 99%, or 100% identical to SEQ ID NO: 158, VL4 comprises an amino acid sequence at least 65%, at least 70%, at least 75%, at least 80%, at least 85%, at least 90%, at least 95%, at least 99%, or 100% identical to SEQ ID NO: 67, and VH4 comprises an amino acid sequence at least 65%, at least 70%, at least 75%, at least 80%, at least 85%, at least 90%, at least 95%, at least 99%, or 100% identical to SEQ ID NO: 71 or 626; or
(t)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 67, VH2 comprises an amino acid sequence at least 65%, at least 70%, at least 75%, at least 80%, at least 85%, at least 90%, at least 95%, at least 99%, or 100% identical to SEQ ID NO: 71 or 626, VL3 comprises an amino acid sequence at least 65%, at least 70%, at least 75%, at least 80%, at least 85%, at least 90%, at least 95%, at least 99%, or 100% identical to SEQ ID NO: 17, VH3 comprises an amino acid sequence at least 65%, at least 70%, at least 75%, at least 80%, at least 85%, at least 90%, at least 95%, at least 99%, or 100% identical to SEQ ID NO: 21 or 22, VL4 comprises an amino acid sequence at least 65%, at least 70%, at least 75%, at least 80%, at least 85%, at least 90%, at least 95%, at least 99%, or 100% identical to SEQ ID NO: 147, and VH4 comprises an amino acid sequence at least 65%, at least 70%, at least 75%, at least 80%, at least 85%, at least 90%, at least 95%, at least 99%, or 100% identical to SEQ ID NO: 151; or
(u)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 67, VH2 comprises an amino acid sequence at least 65%, at least 70%, at least 75%, at least 80%, at least 85%, at least 90%, at least 95%, at least 99%, or 100% identical to SEQ ID NO: 71 or 626, VL3 comprises an amino acid sequence at least 65%, at least 70%, at least 75%, at least 80%, at least 85%, at least 90%, at least 95%, at least 99%, or 100% identical to SEQ ID NO: 147, VH3 comprises an amino acid sequence at least 65%, at least 70%, at least 75%, at least 80%, at least 85%, at least 90%, at least 95%, at least 99%, or 100% identical to SEQ ID NO: 151, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(v)
VL1 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH1 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
VL1 comprises an amino acid sequence at least 65%, at least 70%, at least 75%, at least 80%, at least 85%, at least 90%, at least 95%, at least 99%, or 100% identical to SEQ ID NO: 67, VH1 comprises an amino acid sequence at least 65%, at least 70%, at least 75%, at least 80%, at least 85%, at least 90%, at least 95%, at least 99%, or 100% identical to SEQ ID NO: 71 or 626, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 17, VH3 comprises an amino acid sequence at least 65%, at least 70%, at least 75%, at least 80%, at least 85%, at least 90%, at least 95%, at least 99%, or 100% identical to SEQ ID NO: 21 or 22, VL4 comprises an amino acid sequence at least 65%, at least 70%, at least 75%, at least 80%, at least 85%, at least 90%, at least 95%, at least 99%, or 100% identical to SEQ ID NO: 147, and VH4 comprises an amino acid sequence at least 65%, at least 70%, at least 75%, at least 80%, at least 85%, at least 90%, at least 95%, at least 99%, or 100% identical to SEQ ID NO: 151; or
(w)
VL1 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH1 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
VL1 comprises an amino acid sequence at least 65%, at least 70%, at least 75%, at least 80%, at least 85%, at least 90%, at least 95%, at least 99%, or 100% identical to SEQ ID NO: 67, VH1 comprises an amino acid sequence at least 65%, at least 70%, at least 75%, at least 80%, at least 85%, at least 90%, at least 95%, at least 99%, or 100% identical to SEQ ID NO: 71 or 626, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 147, VH3 comprises an amino acid sequence at least 65%, at least 70%, at least 75%, at least 80%, at least 85%, at least 90%, at least 95%, at least 99%, or 100% identical to SEQ ID NO: 151, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(x)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 17, VH2 comprises an amino acid sequence at least 65%, at least 70%, at least 75%, at least 80%, at least 85%, at least 90%, at least 95%, at least 99%, or 100% identical to SEQ ID NO: 21 or 22, VL3 comprises an amino acid sequence at least 65%, at least 70%, at least 75%, at least 80%, at least 85%, at least 90%, at least 95%, at least 99%, or 100% identical to SEQ ID NO: 67, VH3 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 147, and VH4 comprises an amino acid sequence at least 65%, at least 70%, at least 75%, at least 80%, at least 85%, at least 90%, at least 95%, at least 99%, or 100% identical to SEQ ID NO: 151; or
(y)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 17, VH2 comprises an amino acid sequence at least 65%, at least 70%, at least 75%, at least 80%, at least 85%, at least 90%, at least 95%, at least 99%, or 100% identical to SEQ ID NO: 21 or 22, VL3 comprises an amino acid sequence at least 65%, at least 70%, at least 75%, at least 80%, at least 85%, at least 90%, at least 95%, at least 99%, or 100% identical to SEQ ID NO: 147, VH3 comprises an amino acid sequence at least 65%, at least 70%, at least 75%, at least 80%, at least 85%, at least 90%, at least 95%, at least 99%, or 100% identical to SEQ ID NO: 151, VL4 comprises an amino acid sequence at least 65%, at least 70%, at least 75%, at least 80%, at least 85%, at least 90%, at least 95%, at least 99%, or 100% identical to SEQ ID NO: 67, and VH4 comprises an amino acid sequence at least 65%, at least 70%, at least 75%, at least 80%, at least 85%, at least 90%, at least 95%, at least 99%, or 100% identical to SEQ ID NO: 71 or 626; or
(z)
VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
VL1 comprises an amino acid sequence at least 65%, at least 70%, at least 75%, at least 80%, at least 85%, at least 90%, at least 95%, at least 99%, or 100% identical to SEQ ID NO: 17, VH1 comprises an amino acid sequence at least 65%, at least 70%, at least 75%, at least 80%, at least 85%, at least 90%, at least 95%, at least 99%, or 100% identical to SEQ ID NO: 21 or 22, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 67, VH3 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 147, and VH4 comprises an amino acid sequence at least 65%, at least 70%, at least 75%, at least 80%, at least 85%, at least 90%, at least 95%, at least 99%, or 100% identical to SEQ ID NO: 151; or
(aa)
VL1 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH1 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631, or
VL1 comprises an amino acid sequence at least 65%, at least 70%, at least 75%, at least 80%, at least 85%, at least 90%, at least 95%, at least 99%, or 100% identical to SEQ ID NO: 17, VH1 comprises an amino acid sequence at least 65%, at least 70%, at least 75%, at least 80%, at least 85%, at least 90%, at least 95%, at least 99%, or 100% identical to SEQ ID NO: 21 or 22, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 147, VH3 comprises an amino acid sequence at least 65%, at least 70%, at least 75%, at least 80%, at least 85%, at least 90%, at least 95%, at least 99%, or 100% identical to SEQ ID NO: 151, VL4 comprises an amino acid sequence at least 65%, at least 70%, at least 75%, at least 80%, at least 85%, at least 90%, at least 95%, at least 99%, or 100% identical to SEQ ID NO: 67, and VH4 comprises an amino acid sequence at least 65%, at least 70%, at least 75%, at least 80%, at least 85%, at least 90%, at least 95%, at least 99%, or 100% identical to SEQ ID NO: 71 or 626; or
(bb)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 147, VH2 comprises an amino acid sequence at least 65%, at least 70%, at least 75%, at least 80%, at least 85%, at least 90%, at least 95%, at least 99%, or 100% identical to SEQ ID NO: 151, VL3 comprises an amino acid sequence at least 65%, at least 70%, at least 75%, at least 80%, at least 85%, at least 90%, at least 95%, at least 99%, or 100% identical to SEQ ID NO: 67, VH3 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(cc)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL3 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH3 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL4 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 147, VH2 comprises an amino acid sequence at least 65%, at least 70%, at least 75%, at least 80%, at least 85%, at least 90%, at least 95%, at least 99%, or 100% identical to SEQ ID NO: 151, VL3 comprises an amino acid sequence at least 65%, at least 70%, at least 75%, at least 80%, at least 85%, at least 90%, at least 95%, at least 99%, or 100% identical to SEQ ID NO: 17, VH3 comprises an amino acid sequence at least 65%, at least 70%, at least 75%, at least 80%, at least 85%, at least 90%, at least 95%, at least 99%, or 100% identical to SEQ ID NO: 21 or 22, VL4 comprises an amino acid sequence at least 65%, at least 70%, at least 75%, at least 80%, at least 85%, at least 90%, at least 95%, at least 99%, or 100% identical to SEQ ID NO: 67, and VH4 comprises an amino acid sequence at least 65%, at least 70%, at least 75%, at least 80%, at least 85%, at least 90%, at least 95%, at least 99%, or 100% identical to SEQ ID NO: 71 or 626; or
(dd)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 67, VH2 comprises an amino acid sequence at least 65%, at least 70%, at least 75%, at least 80%, at least 85%, at least 90%, at least 95%, at least 99%, or 100% identical to SEQ ID NO: 71 or 626, VL3 comprises an amino acid sequence at least 65%, at least 70%, at least 75%, at least 80%, at least 85%, at least 90%, at least 95%, at least 99%, or 100% identical to SEQ ID NO: 147, VH3 comprises an amino acid sequence at least 65%, at least 70%, at least 75%, at least 80%, at least 85%, at least 90%, at least 95%, at least 99%, or 100% identical to SEQ ID NO: 151, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(ee)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 15, a CDR2 having an amino acid sequence at least 65%, at least 70%, at least 75%, at least 80%, at least 85%, at least 90%, at least 95%, at least 99%, or 100% identical to amino acid sequence LGS, and a CDR3 having an amino acid sequence at least 65%, at least 70%, at least 75%, at least 80%, at least 85%, at least 90%, at least 95%, at least 99%, or 100% identical to SEQ ID NO: 16,
VH2 comprises a CDR1 having an amino acid sequence at least 65%, at least 70%, at least 75%, at least 80%, at least 85%, at least 90%, at least 95%, at least 99%, or 100% identical to SEQ ID NO: 18, a CDR2 having an amino acid sequence at least 65%, at least 70%, at least 75%, at least 80%, at least 85%, at least 90%, at least 95%, at least 99%, or 100% identical to SEQ ID NO: 19, and a CDR3 having an amino acid sequence at least 65%, at least 70%, at least 75%, at least 80%, at least 85%, at least 90%, at least 95%, at least 99%, or 100% identical to SEQ ID NO: 20,
VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 17, VH2 comprises an amino acid sequence at least 65%, at least 70%, at least 75%, at least 80%, at least 85%, at least 90%, at least 95%, at least 99%, or 100% identical to SEQ ID NO: 21 or 22, VL3 comprises an amino acid sequence at least 65%, at least 70%, at least 75%, at least 80%, at least 85%, at least 90%, at least 95%, at least 99%, or 100% identical to SEQ ID NO: 67, VH3 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 147, and VH4 comprises an amino acid sequence at least 65%, at least 70%, at least 75%, at least 80%, at least 85%, at least 90%, at least 95%, at least 99%, or 100% identical to SEQ ID NO: 151; or
(ff)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH2 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 67, VH2 comprises an amino acid sequence at least 65%, at least 70%, at least 75%, at least 80%, at least 85%, at least 90%, at least 95%, at least 99%, or 100% identical to SEQ ID NO: 71 or 626, VL3 comprises an amino acid sequence at least 65%, at least 70%, at least 75%, at least 80%, at least 85%, at least 90%, at least 95%, at least 99%, or 100% identical to SEQ ID NO: 147, VH3 comprises an amino acid sequence at least 65%, at least 70%, at least 75%, at least 80%, at least 85%, at least 90%, at least 95%, at least 99%, or 100% identical to SEQ ID NO: 151, VL4 comprises an amino acid sequence at least 65%, at least 70%, at least 75%, at least 80%, at least 85%, at least 90%, at least 95%, at least 99%, or 100% identical to SEQ ID NO: 154, and VH4 comprises an amino acid sequence at least 65%, at least 70%, at least 75%, at least 80%, at least 85%, at least 90%, at least 95%, at least 99%, or 100% identical to SEQ ID NO: 158; or
(gg)
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766,
VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049,
VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 154, VH2 comprises an amino acid sequence at least 65%, at least 70%, at least 75%, at least 80%, at least 85%, at least 90%, at least 95%, at least 99%, or 100% identical to SEQ ID NO: 158, VL3 comprises an amino acid sequence at least 65%, at least 70%, at least 75%, at least 80%, at least 85%, at least 90%, at least 95%, at least 99%, or 100% identical to SEQ ID NO: 67, VH3 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 147, and VH4 comprises an amino acid sequence at least 65%, at least 70%, at least 75%, at least 80%, at least 85%, at least 90%, at least 95%, at least 99%, or 100% identical to SEQ ID NO: 151; or
(hh)
VL1 comprises the same light chain variable region as VL3, VH1 comprises the same heavy chain variable region as VH3, VL2 comprises the same light chain variable region as VL4, and VH2 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766,
VH1 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049,
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766,
VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 154, VH1 and VH3 each comprise an amino acid sequence at least 65%, at least 70%, at least 75%, at least 80%, at least 85%, at least 90%, at least 95%, at least 99%, or 100% identical to SEQ ID NO: 158, VL2 and VL4 each comprise an amino acid sequence at least 65%, at least 70%, at least 75%, at least 80%, at least 85%, at least 90%, at least 95%, at least 99%, or 100% identical to SEQ ID NO: 147, and VH2 and VH4 each comprise an amino acid sequence at least 65%, at least 70%, at least 75%, at least 80%, at least 85%, at least 90%, at least 95%, at least 99%, or 100% identical to SEQ ID NO: 151; or
(ii)
VL1 comprises the same light chain variable region as VL3, VH1 comprises the same heavy chain variable region as VH3, VL2 comprises the same light chain variable region as VL4, and VH2 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 147, and VH2 and VH4 each comprise an amino acid sequence at least 65%, at least 70%, at least 75%, at least 80%, at least 85%, at least 90%, at least 95%, at least 99%, or 100% identical to SEQ ID NO: 151; or
(jj)
VL2 comprises the same light chain variable region as VL3, and VH2 comprises the same heavy chain variable region as VH3, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049, or
wherein:
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 and VL3 each comprise an amino acid sequence at least 65%, at least 70%, at least 75%, at least 80%, at least 85%, at least 90%, at least 95%, at least 99%, or 100% identical to SEQ ID NO: 147, VH2 and VH3 each comprise an amino acid sequence at least 65%, at least 70%, at least 75%, at least 80%, at least 85%, at least 90%, at least 95%, at least 99%, or 100% identical to SEQ ID NO: 151, VL4 comprises an amino acid sequence at least 65%, at least 70%, at least 75%, at least 80%, at least 85%, at least 90%, at least 95%, at least 99%, or 100% identical to SEQ ID NO: 154, and VH4 comprises an amino acid sequence at least 65%, at least 70%, at least 75%, at least 80%, at least 85%, at least 90%, at least 95%, at least 99%, or 100% identical to SEQ ID NO: 158; or
(kk)
VL2 comprises the same light chain variable region as VL4, and VH2 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766,
VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
wherein:
VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 147, VH2 and VH4 each comprise an amino acid sequence at least 65%, at least 70%, at least 75%, at least 80%, at least 85%, at least 90%, at least 95%, at least 99%, or 100% identical to SEQ ID NO: 151, VL3 comprises an amino acid sequence at least 65%, at least 70%, at least 75%, at least 80%, at least 85%, at least 90%, at least 95%, at least 99%, or 100% identical to SEQ ID NO: 154, and VH3 comprises an amino acid sequence at least 65%, at least 70%, at least 75%, at least 80%, at least 85%, at least 90%, at least 95%, at least 99%, or 100% identical to SEQ ID NO: 158; or
(ll)
VL1 comprises the same light chain variable region as VL3, and VH1 comprises the same heavy chain variable region as VH3, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049, or
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 147, VH1 and VH3 each comprise an amino acid sequence at least 65%, at least 70%, at least 75%, at least 80%, at least 85%, at least 90%, at least 95%, at least 99%, or 100% identical to SEQ ID NO: 151, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL4 comprises an amino acid sequence at least 65%, at least 70%, at least 75%, at least 80%, at least 85%, at least 90%, at least 95%, at least 99%, or 100% identical to SEQ ID NO: 154, and VH4 comprises an amino acid sequence at least 65%, at least 70%, at least 75%, at least 80%, at least 85%, at least 90%, at least 95%, at least 99%, or 100% identical to SEQ ID NO: 158; or
(mm)
VL1 comprises the same light chain variable region as VL4, and VH1 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766,
VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049.
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
wherein:
VL1 and VL4 each comprise an amino acid sequence at least 65%, at least 70%, at least 75%, at least 80%, at least 85%, at least 90%, at least 95%, at least 99%, or 100% identical to SEQ ID NO: 147, VH1 and VH4 each comprise an amino acid sequence at least 65%, at least 70%, at least 75%, at least 80%, at least 85%, at least 90%, at least 95%, at least 99%, or 100% identical to SEQ ID NO: 151, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 154, and VH3 comprises an amino acid sequence at least 65%, at least 70%, at least 75%, at least 80%, at least 85%, at least 90%, at least 95%, at least 99%, or 100% identical to SEQ ID NO: 158; or
(nn)
VL1 comprises the same light chain variable region as VL3, VH1 comprises the same heavy chain variable region as VH3, VL2 comprises the same light chain variable region as VL4, and VH2 comprises the same heavy chain variable region as VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792,
VH1 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064,
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL3 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792,
VH3 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 174, VH1 and VH3 each comprise an amino acid sequence at least 65%, at least 70%, at least 75%, at least 80%, at least 85%, at least 90%, at least 95%, at least 99%, or 100% identical to SEQ ID NO: 178, VL2 and VL4 each comprise an amino acid sequence at least 65%, at least 70%, at least 75%, at least 80%, at least 85%, at least 90%, at least 95%, at least 99%, or 100% identical to SEQ ID NO: 147, and VH2 and VH4 each comprise an amino acid sequence at least 65%, at least 70%, at least 75%, at least 80%, at least 85%, at least 90%, at least 95%, at least 99%, or 100% identical to SEQ ID NO: 151; or
(oo)
VL2 comprises the same light chain variable region as VL3, and VH2 comprises the same heavy chain variable region as VH3, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792,
VH1 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064.
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049, or
wherein:
VL1 comprises an amino acid sequence at least 65%, at least 70%, at least 75%, at least 80%, at least 85%, at least 90%, at least 95%, at least 99%, or 100% identical to SEQ ID NO: 174, VH1 comprises an amino acid sequence at least 65%, at least 70%, at least 75%, at least 80%, at least 85%, at least 90%, at least 95%, at least 99%, or 100% identical to SEQ ID NO: 178, VL2 and VL3 each comprise an amino acid sequence at least 65%, at least 70%, at least 75%, at least 80%, at least 85%, at least 90%, at least 95%, at least 99%, or 100% identical to SEQ ID NO: 147, VH2 and VH3 each comprise an amino acid sequence at least 65%, at least 70%, at least 75%, at least 80%, at least 85%, at least 90%, at least 95%, at least 99%, or 100% identical to SEQ ID NO: 151, VL4 comprises an amino acid sequence at least 65%, at least 70%, at least 75%, at least 80%, at least 85%, at least 90%, at least 95%, at least 99%, or 100% identical to SEQ ID NO: 154, and VH4 comprises an amino acid sequence at least 65%, at least 70%, at least 75%, at least 80%, at least 85%, at least 90%, at least 95%, at least 99%, or 100% identical to SEQ ID NO: 158; or
(pp)
VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792,
VH2 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064,
VL3 comprises a CDR1 having an amino acid sequence at least 65%, at least 70%, at least 75%, at least 80%, at least 85%, at least 90%, at least 95%, at least 99%, or 100% identical to SEQ ID NO: 65 or 627, a CDR2 having an amino acid sequence at least 65%, at least 70%, at least 75%, at least 80%, at least 85%, at least 90%, at least 95%, at least 99%, or 100% identical to SEQ ID NO: 628 or an amino acid sequence at least 65%, at least 70%, at least 75%, at least 80%, at least 85%, at least 90%, at least 95%, at least 99%, or 100% identical to amino acid sequence SDS, and a CDR3 having an amino acid sequence at least 65%, at least 70%, at least 75%, at least 80%, at least 85%, at least 90%, at least 95%, at least 99%, or 100% identical to SEQ ID NO: 66,
VH3 comprises a CDR1 having an amino acid sequence at least 65%, at least 70%, at least 75%, at least 80%, at least 85%, at least 90%, at least 95%, at least 99%, or 100% identical to SEQ ID NO: 68 or 629, a CDR2 having an amino acid sequence at least 65%, at least 70%, at least 75%, at least 80%, at least 85%, at least 90%, at least 95%, at least 99%, or 100% identical to SEQ ID NO: 69 or 630, and a CDR3 having an amino acid sequence at least 65%, at least 70%, at least 75%, at least 80%, at least 85%, at least 90%, at least 95%, at least 99%, or 100% identical to SEQ ID NO: 70 or 631,
VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049, or
VL1 comprises an amino acid sequence at least 65%, at least 70%, at least 75%, at least 80%, at least 85%, at least 90%, at least 95%, at least 99%, or 100% identical to SEQ ID NO: 147, VH1 comprises an amino acid sequence at least 65%, at least 70%, at least 75%, at least 80%, at least 85%, at least 90%, at least 95%, at least 99%, or 100% identical to SEQ ID NO: 151, VL2 comprises an amino acid sequence at least 65%, at least 70%, at least 75%, at least 80%, at least 85%, at least 90%, at least 95%, at least 99%, or 100% identical to SEQ ID NO: 174, VH2 comprises an amino acid sequence at least 65%, at least 70%, at least 75%, at least 80%, at least 85%, at least 90%, at least 95%, at least 99%, or 100% identical to SEQ ID NO: 178, VL3 comprises an amino acid sequence at least 65%, at least 70%, at least 75%, at least 80%, at least 85%, at least 90%, at least 95%, at least 99%, or 100% identical to SEQ ID NO: 67, VH3 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 154, and VH4 comprises an amino acid sequence at least 65%, at least 70%, at least 75%, at least 80%, at least 85%, at least 90%, at least 95%, at least 99%, or 100% identical to SEQ ID NO: 158; or
(qq)
VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792,
VH2 comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064,
VL3 comprises a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066,
VH3 comprises a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069,
VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049, or
VL1 comprises an amino acid sequence at least 65%, at least 70%, at least 75%, at least 80%, at least 85%, at least 90%, at least 95%, at least 99%, or 100% identical to SEQ ID NO: 147, VH1 comprises an amino acid sequence at least 65%, at least 70%, at least 75%, at least 80%, at least 85%, at least 90%, at least 95%, at least 99%, or 100% identical to SEQ ID NO: 151, VL2 comprises an amino acid sequence at least 65%, at least 70%, at least 75%, at least 80%, at least 85%, at least 90%, at least 95%, at least 99%, or 100% identical to SEQ ID NO: 174, VH2 comprises an amino acid sequence at least 65%, at least 70%, at least 75%, at least 80%, at least 85%, at least 90%, at least 95%, at least 99%, or 100% identical to SEQ ID NO: 178, VL3 comprises an amino acid sequence at least 65%, at least 70%, at least 75%, at least 80%, at least 85%, at least 90%, at least 95%, at least 99%, or 100% identical to SEQ ID NO: 140, VH3 comprises an amino acid sequence at least 65%, at least 70%, at least 75%, at least 80%, at least 85%, at least 90%, at least 95%, at least 99%, or 100% identical to SEQ ID NO: 144, VL4 comprises an amino acid sequence at least 65%, at least 70%, at least 75%, at least 80%, at least 85%, at least 90%, at least 95%, at least 99%, or 100% identical to SEQ ID NO: 154, and VH4 comprises an amino acid sequence at least 65%, at least 70%, at least 75%, at least 80%, at least 85%, at least 90%, at least 95%, at least 99%, or 100% identical to SEQ ID NO: 158; or
(rr)
VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 138 or 1065, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 139, 856, or 1066,
VH3 comprises a CDR1 having an amino acid sequence at least 65%, at least 70%, at least 75%, at least 80%, at least 85%, at least 90%, at least 95%, at least 99%, or 100% identical to SEQ ID NO: 141 or 1067, a CDR2 having an amino acid sequence at least 65%, at least 70%, at least 75%, at least 80%, at least 85%, at least 90%, at least 95%, at least 99%, or 100% identical to SEQ ID NO: 142 or 1068, and a CDR3 having an amino acid sequence at least 65%, at least 70%, at least 75%, at least 80%, at least 85%, at least 90%, at least 95%, at least 99%, or 100% identical to SEQ ID NO: 143, 857, or 1069,
VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049, or
VL1 comprises an amino acid sequence at least 65%, at least 70%, at least 75%, at least 80%, at least 85%, at least 90%, at least 95%, at least 99%, or 100% identical to SEQ ID NO: 147, VH1 comprises an amino acid sequence at least 65%, at least 70%, at least 75%, at least 80%, at least 85%, at least 90%, at least 95%, at least 99%, or 100% identical to SEQ ID NO: 151, VL2 comprises an amino acid sequence at least 65%, at least 70%, at least 75%, at least 80%, at least 85%, at least 90%, at least 95%, at least 99%, or 100% identical to SEQ ID NO: 11, VH2 comprises an amino acid sequence at least 65%, at least 70%, at least 75%, at least 80%, at least 85%, at least 90%, at least 95%, at least 99%, or 100% identical to SEQ ID NO: 14, VL3 comprises an amino acid sequence at least 65%, at least 70%, at least 75%, at least 80%, at least 85%, at least 90%, at least 95%, at least 99%, or 100% identical to SEQ ID NO: 140, VH3 comprises an amino acid sequence at least 65%, at least 70%, at least 75%, at least 80%, at least 85%, at least 90%, at least 95%, at least 99%, or 100% identical to SEQ ID NO: 144, VL4 comprises an amino acid sequence at least 65%, at least 70%, at least 75%, at least 80%, at least 85%, at least 90%, at least 95%, at least 99%, or 100% identical to SEQ ID NO: 154, and VH4 comprises an amino acid sequence at least 65%, at least 70%, at least 75%, at least 80%, at least 85%, at least 90%, at least 95%, at least 99%, or 100% identical to SEQ ID NO: 158, or
(ss)
VL1 comprises the same light chain variable region as VL2, VL3, and VL4, and VH1 comprises the same heavy chain variable region as VH2, VH3, and VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH1 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL3 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764,
VH3 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054,
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054, or
wherein:
VL1, VL2, VL3, and VL4 each comprise an amino acid sequence at least 65%, at least 70%, at least 75%, at least 80%, at least 85%, at least 90%, at least 95%, at least 99%, or 100% identical to SEQ ID NO: 147, and VH1, VH2, VH3, and VH4 each comprise an amino acid sequence at least 65%, at least 70%, at least 75%, at least 80%, at least 85%, at least 90%, at least 95%, at least 99%, or 100% identical to SEQ ID NO: 151; or
(tt)
VL1 comprises the same light chain variable region as VL2, VL3, and VL4, and VH1 comprises the same heavy chain variable region as VH2, VH3, and VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766,
VH1 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049,
VL2 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766,
VH2 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049,
VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766,
VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049,
VL4 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049, or
wherein:
VL1, VL2, VL3, and VL4 each comprise an amino acid sequence at least 65%, at least 70%, at least 75%, at least 80%, at least 85%, at least 90%, at least 95%, at least 99%, or 100% identical to SEQ ID NO: 154, and VH1, VH2, VH3, and VH4 each comprise an amino acid sequence at least 65%, at least 70%, at least 75%, at least 80%, at least 85%, at least 90%, at least 95%, at least 99%, or 100% identical to SEQ ID NO: 158; or
(uu)
VL1 comprises the same light chain variable region as VL2, VL3, and VL4, and VH1 comprises the same heavy chain variable region as VH2, VH3, and VH4, and
wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH1 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL2 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH2 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL3 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10,
VH3 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059,
VL4 comprises a CDR1 having an amino acid sequence at least 65%, at least 70%, at least 75%, at least 80%, at least 85%, at least 90%, at least 95%, at least 99%, or 100% identical to SEQ ID NO: 9 or 1055, a CDR2 having an amino acid sequence at least 65%, at least 70%, at least 75%, at least 80%, at least 85%, at least 90%, at least 95%, at least 99%, or 100% identical to SEQ ID NO: 1056 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 10, and
VH4 comprises a CDR1 having an amino acid sequence at least 65%, at least 70%, at least 75%, at least 80%, at least 85%, at least 90%, at least 95%, at least 99%, or 100% identical to SEQ ID NO: 12 or 1057, a CDR2 having an amino acid sequence at least 65%, at least 70%, at least 75%, at least 80%, at least 85%, at least 90%, at least 95%, at least 99%, or 100% identical to SEQ ID NO: 6 or 1058, and a CDR3 having an amino acid sequence at least 65%, at least 70%, at least 75%, at least 80%, at least 85%, at least 90%, at least 95%, at least 99%, or 100% identical to SEQ ID NO: 13 or 1059, or
wherein:
VL1, VL2, VL3, and VL4 each comprise an amino acid sequence at least 65%, at least 70%, at least 75%, at least 80%, at least 85%, at least 90%, at least 95%, at least 99%, or 100% identical to SEQ ID NO: 11, and VH1, VH2, VH3, and VH4 each comprise an amino acid sequence at least 65%, at least 70%, at least 75%, at least 80%, at least 85%, at least 90%, at least 95%, at least 99%, or 100% identical to SEQ ID NO: 14.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOS: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof. (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the first polypeptide comprises a structure represented by VL1-VH1-VL2-CL-VH2-CH1, VL1-L1-VH1-L2-VL2-L3-CL-L4-VH2-L5-CH1, VL1-VH1-VL2-CL-VH2-CH1-CH2-CH3, or VL1-L1-VH1-L2-VL2-L3-CL-L4-VH2-L5-CH1-CH2-CH3 and the second polypeptide comprises a structure represented by VL3-VH3-VL4-CL-VH4-CH1, VL3-L6-VH3-L7-VL4-L8-CL-L9-VH4-L10-CH1, VL3-VH3-VL4-CL-VH4-CH1-CH2-CH3, or VL3-L6-VH3-L7-VL4-L8-CL-L9-VH4-L10-CH1-CH2-CH3
wherein:
(a) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 17, and VH2 and VH4 each comprise an amino acid sequence at least 65%, at least 70%, at least 75%, at least 80%, at least 85%, at least 90%, at least 95%, at least 99%, or 100% identical to SEQ ID NO: 21 or 22; or
(b) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 17, VH1 and VH3 each comprise an amino acid sequence at least 65%, at least 70%, at least 75%, at least 80%, at least 85%, at least 90%, at least 95%, at least 99%, or 100% identical to SEQ ID NO: 21 or 22, VL2 and VL4 each comprise an amino acid sequence at least 65%, at least 70%, at least 75%, at least 80%, at least 85%, at least 90%, at least 95%, at least 99%, or 100% identical to SEQ ID NO: 11, and VH2 and VH4 each comprise an amino acid sequence at least 65%, at least 70%, at least 75%, at least 80%, at least 85%, at least 90%, at least 95%, at least 99%, or 100% identical to SEQ ID NO: 14; or
(c) VL1 comprises the same light chain variable region as VL4, VH1 comprises the same heavy chain variable region as VH4, and
wherein:
VL1 and VL4 each comprise an amino acid sequence at least 65%, at least 70%, at least 75%, at least 80%, at least 85%, at least 90%, at least 95%, at least 99%, or 100% identical to SEQ ID NO: 11, VH1 and VH4 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22; or
(d) VL1 comprises an amino acid sequence at least 65%, at least 70%, at least 75%, at least 80%, at least 85%, at least 90%, at least 95%, at least 99%, or 100% identical to SEQ ID NO: 4, VH1 comprises an amino acid sequence at least 65%, at least 70%, at least 75%, at least 80%, at least 85%, at least 90%, at least 95%, at least 99%, or 100% identical to SEQ ID NO: 8, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 33 or 39, VH3 comprises an amino acid sequence at least 65%, at least 70%, at least 75%, at least 80%, at least 85%, at least 90%, at least 95%, at least 99%, or 100% identical to SEQ ID NO: 37, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOS: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the first polypeptide comprises a structure represented by VL1-CL-VH1-CH1-VL2-VH2, VL1-L1-CL-L2-VH1-L3-CH1-L4-VL2-L5-VH2, VL1-CL-VH1-CH1-VL2-VH2-CH2-CH3, or VL1-L1-CL-L2-VH1-L3-CH1-L4-VL2-L5-VH2-CH2-CH3 and the second polypeptide comprises a structure represented by VL3-CL-VH3-CH1-VL4-VH4, VL3-L6-CL-L7-VH3-L8-CH1-L9-VL4-L10-VH4, VL3-CL-VH3-CH1-VL4-VH4-CH2-CH3, or VL3-L6-CL-L7-VH3-L8-CH1-L9-VL4-L10-VH4-CH2-CH3,
wherein:
(a) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 17, and VH2 and VH4 each comprise an amino acid sequence at least 65%, at least 70%, at least 75%, at least 80%, at least 85%, at least 90%, at least 95%, at least 99%, or 100% identical to SEQ ID NO: 21 or 22; or
(b) VL1 comprises the same light chain variable region as VL4, VH1 comprises the same heavy chain variable region as VH4, and
wherein:
VL1 and VL4 each comprise an amino acid sequence at least 65%, at least 70%, at least 75%, at least 80%, at least 85%, at least 90%, at least 95%, at least 99%, or 100% identical to SEQ ID NO: 11, VH1 and VH4 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22; or
(c) VL1 comprises an amino acid sequence at least 65%, at least 70%, at least 75%, at least 80%, at least 85%, at least 90%, at least 95%, at least 99%, or 100% identical to SEQ ID NO: 4, VH1 comprises an amino acid sequence at least 65%, at least 70%, at least 75%, at least 80%, at least 85%, at least 90%, at least 95%, at least 99%, or 100% identical to SEQ ID NO: 8, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 33 or 39, VH3 comprises an amino acid sequence at least 65%, at least 70%, at least 75%, at least 80%, at least 85%, at least 90%, at least 95%, at least 99%, or 100% identical to SEQ ID NO: 37, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, the first polypeptide comprises a structure represented by VL1-VL2-VH2-VH1-CL-CH1, VL1-L1-VL2-L2-VH2-L3-VH1-L4-CL-L5-CH1, VL1-VL2-VH2-VH1-CL-CH1-CH2-CH3, or VL1-L1-VL2-L2-VH2-L3-VH1-L4-CL-L5-CH1-CH2-CH3 and the second polypeptide comprises a structure represented by VL3-VL4-VH4-VH3-CL-CH1, VL3-L6-VL4-L7-VH4-L8-VH3-L9-CL-L10-CH1, VL3-VL4-VH4-VH3-CL-CH1-CH2-CH3, or VL3-L6-VL4-L7-VH4-L8-VH3-L9-CL-L10-CH1-CH2-CH3,
wherein:
(a) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 17, and VH2 and VH4 each comprise an amino acid sequence at least 65%, at least 70%, at least 75%, at least 80%, at least 85%, at least 90%, at least 95%, at least 99%, or 100% identical to SEQ ID NO: 21 or 22; or
(b) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 17, and VH2 and VH4 each comprise an amino acid sequence at least 65%, at least 70%, at least 75%, at least 80%, at least 85%, at least 90%, at least 95%, at least 99%, or 100% identical to SEQ ID NO: 21 or 22; or
(c) VL1 comprises the same light chain variable region as VL4, VH1 comprises the same heavy chain variable region as VH4, VL2 comprises the same light chain variable region as VL3, VH2 comprises the same heavy chain variable region as VH3, and
wherein:
VL1 and VL4 each comprise an amino acid sequence at least 65%, at least 70%, at least 75%, at least 80%, at least 85%, at least 90%, at least 95%, at least 99%, or 100% identical to SEQ ID NO: 17, VH1 and VH4 each comprise an amino acid sequence at least 65%, at least 70%, at least 75%, at least 80%, at least 85%, at least 90%, at least 95%, at least 99%, or 100% identical to SEQ ID NO: 21 or 22, VL2 and VL3 each comprise an amino acid sequence at least 65%, at least 70%, at least 75%, at least 80%, at least 85%, at least 90%, at least 95%, at least 99%, or 100% identical to SEQ ID NO: 11, and VH2 and VH3 each comprise an amino acid sequence at least 65%, at least 70%, at least 75%, at least 80%, at least 85%, at least 90%, at least 95%, at least 99%, or 100% identical to SEQ ID NO: 14; or
(d) VL1 comprises the same light chain variable region as VL4, VH1 comprises the same heavy chain variable region as VH4, VL2 comprises the same light chain variable region as VL3, VH2 comprises the same heavy chain variable region as VH3, and
wherein:
VL1 and VL4 each comprise an amino acid sequence at least 65%, at least 70%, at least 75%, at least 80%, at least 85%, at least 90%, at least 95%, at least 99%, or 100% identical to SEQ ID NO: 11, VH1 and VH4 each comprise an amino acid sequence at least 65%, at least 70%, at least 75%, at least 80%, at least 85%, at least 90%, at least 95%, at least 99%, or 100% identical to SEQ ID NO: 14, VL2 and VL3 each comprise an amino acid sequence at least 65%, at least 70%, at least 75%, at least 80%, at least 85%, at least 90%, at least 95%, at least 99%, or 100% identical to SEQ ID NO: 17, and VH2 and VH3 each comprise an amino acid sequence at least 65%, at least 70%, at least 75%, at least 80%, at least 85%, at least 90%, at least 95%, at least 99%, or 100% identical to SEQ ID NO: 21 or 22; or
(e) VL2 comprises the same light chain variable region as VL3, VH2 comprises the same heavy chain variable region as VH3, and
wherein:
VL1 comprises an amino acid sequence at least 65%, at least 70%, at least 75%, at least 80%, at least 85%, at least 90%, at least 95%, at least 99%, or 100% identical to SEQ ID NO: 25, VH1 comprises an amino acid sequence at least 65%, at least 70%, at least 75%, at least 80%, at least 85%, at least 90%, at least 95%, at least 99%, or 100% identical to SEQ ID NO: 29, VL2 and VL3 each comprise an amino acid sequence at least 65%, at least 70%, at least 75%, at least 80%, at least 85%, at least 90%, at least 95%, at least 99%, or 100% identical to SEQ ID NO: 11, VH2 and VH3 each comprise an amino acid sequence at least 65%, at least 70%, at least 75%, at least 80%, at least 85%, at least 90%, at least 95%, at least 99%, or 100% identical to SEQ ID NO: 14, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(f) VL1 comprises the same light chain variable region as VL3, VH1 comprises the same heavy chain variable region as VH3, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22; or
(g) VL1 comprises the same light chain variable region as VL4, VH1 comprises the same heavy chain variable region as VH4, and
wherein:
VL1 and VL4 each comprise an amino acid sequence at least 65%, at least 70%, at least 75%, at least 80%, at least 85%, at least 90%, at least 95%, at least 99%, or 100% identical to SEQ ID NO: 11, VH1 and VH4 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22; or
(h) VL1 comprises the same light chain variable region as VL3, VH1 comprises the same heavy chain variable region as VH3, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 67, VH2 comprises an amino acid sequence at least 65%, at least 70%, at least 75%, at least 80%, at least 85%, at least 90%, at least 95%, at least 99%, or 100% identical to SEQ ID NO: 71 or 626, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOS: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOS: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the first polypeptide comprises a structure represented by VL1-VH1-VL2-CL-VH2-CH1, VL1-L1-VH1-L2-VL2-L3-CL-L4-VH2-L5-CH1, VL1-VH1-VL2-CL-VH2-CH1-CH2-CH3, or VL1-L1-VH1-L2-VL2-L3-CL-L4-VH2-L5-CH1-CH2-CH3 and the second polypeptide comprises a structure represented by VL3-CL-VH3-CH1-VL4-VH4, VL3-L6-CL-L7-VH3-L8-CH1-L9-VL4-L10-VH4, VL3-CL-VH3-CH1-VL4-VH4-CH2-CH3, or VL3-L6-CL-L7-VH3-L8-CH1-L9-VL4-L10-VH4-CH2-CH3,
wherein:
(a) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 17, and VH2 and VH4 each comprise an amino acid sequence at least 65%, at least 70%, at least 75%, at least 80%, at least 85%, at least 90%, at least 95%, at least 99%, or 100% identical to SEQ ID NO: 21 or 22; or
(b) VL1 comprises the same light chain variable region as VL4, VH1 comprises the same heavy chain variable region as VH4, and
wherein:
VL1 and VL4 each comprise an amino acid sequence at least 65%, at least 70%, at least 75%, at least 80%, at least 85%, at least 90%, at least 95%, at least 99%, or 100% identical to SEQ ID NO: 11, VH1 and VH4 each comprise an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 17, and VH3 comprises an amino acid sequence at least 65%, at least 70%, at least 75%, at least 80%, at least 85%, at least 90%, at least 95%, at least 99%, or 100% identical to SEQ ID NO: 21 or 22; or
(c) VL1 comprises an amino acid sequence at least 65%, at least 70%, at least 75%, at least 80%, at least 85%, at least 90%, at least 95%, at least 99%, or 100% identical to SEQ ID NO: 4, VH1 comprises an amino acid sequence at least 65%, at least 70%, at least 75%, at least 80%, at least 85%, at least 90%, at least 95%, at least 99%, or 100% identical to SEQ ID NO: 8, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 33, 34 or 39, VH3 comprises an amino acid sequence at least 65%, at least 70%, at least 75%, at least 80%, at least 85%, at least 90%, at least 95%, at least 99%, or 100% identical to SEQ ID NO: 37, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, the first polypeptide comprises a structure represented by VL1-CL-VH1-CH1-VL2-VH2, VL1-L1-CL-L2-VH1-L3-CH1-L4-VL2-L5-VH2, VL1-CL-VH1-CH1-VL2-VH2-CH2-CH3, or VL1-L1-CL-L2-VH1-L3-CH1-L4-VL2-L5-VH2-CH2-CH3 and the second polypeptide comprises a structure represented by VL3-VH3-VL4-CL-VH4-CH1, VL3-L6-VH3-L7-VL4-L8-CL-L9-VH4-L10-CH1, VL3-VH3-VL4-CL-VH4-CH1-CH2-CH3, or VL3-L6-VH3-L7-VL4-L8-CL-L9-VH4-L10-CH1-CH2-CH3.
wherein:
(a) VL1 comprises the same light chain variable region as VL3, VH1 comprises the same heavy chain variable region as VH3, VL2 comprises the same light chain variable region as VL4, VH2 comprises the same heavy chain variable region as VH4, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 11, VH1 and VH3 each comprise an amino acid sequence at least 65%, at least 70%, at least 75%, at least 80%, at least 85%, at least 90%, at least 95%, at least 99%, or 100% identical to SEQ ID NO: 14, VL2 and VL4 each comprise an amino acid sequence at least 65%, at least 70%, at least 75%, at least 80%, at least 85%, at least 90%, at least 95%, at least 99%, or 100% identical to SEQ ID NO: 17, and VH2 and VH4 each comprise an amino acid sequence at least 65%, at least 70%, at least 75%, at least 80%, at least 85%, at least 90%, at least 95%, at least 99%, or 100% identical to SEQ ID NO: 21 or 22; or
(b) VL1 comprises the same light chain variable region as VL3, VH1 comprises the same heavy chain variable region as VH3, and
wherein:
VL1 and VL3 each comprise an amino acid sequence at least 65%, at least 70%, at least 75%, at least 80%, at least 85%, at least 90%, at least 95%, at least 99%, or 100% identical to SEQ ID NO: 17, VH1 and VH3 each comprise an amino acid sequence at least 65%, at least 70%, at least 75%, at least 80%, at least 85%, at least 90%, at least 95%, at least 99%, or 100% identical to SEQ ID NO: 21 or 22, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL4 comprises an amino acid sequence at least 65%, at least 70%, at least 75%, at least 80%, at least 85%, at least 90%, at least 95%, at least 99%, or 100% identical to SEQ ID NO: 11, and VH4 comprises an amino acid sequence at least 65%, at least 70%, at least 75%, at least 80%, at least 85%, at least 90%, at least 95%, at least 99%, or 100% identical to SEQ ID NO: 14; or
(c) VL1 comprises an amino acid sequence at least 65%, at least 70%, at least 75%, at least 80%, at least 85%, at least 90%, at least 95%, at least 99%, or 100% identical to SEQ ID NO: 4, VH1 comprises an amino acid sequence at least 65%, at least 70%, at least 75%, at least 80%, at least 85%, at least 90%, at least 95%, at least 99%, or 100% identical to SEQ ID NO: 8, VL2 comprises an amino acid sequence at least 65%, at least 70%, at least 75%, at least 80%, at least 85%, at least 90%, at least 95%, at least 99%, or 100% identical to SEQ ID NO: 25, VH2 comprises an amino acid sequence at least 65%, at least 70%, at least 75%, at least 80%, at least 85%, at least 90%, at least 95%, at least 99%, or 100% identical to SEQ ID NO: 29, VL3 comprises an amino acid sequence at least 65%, at least 70%, at least 75%, at least 80%, at least 85%, at least 90%, at least 95%, at least 99%, or 100% identical to SEQ ID NO: 33 or 39, VH3 comprises an amino acid sequence at least 65%, at least 70%, at least 75%, at least 80%, at least 85%, at least 90%, at least 95%, at least 99%, or 100% identical to SEQ ID NO: 37, VL4 comprises an amino acid sequence at least 65%, at least 70%, at least 75%, at least 80%, at least 85%, at least 90%, at least 95%, at least 99%, or 100% identical to SEQ ID NO: 17, and VH4 comprises an amino acid sequence at least 65%, at least 70%, at least 75%, at least 80%, at least 85%, at least 90%, at least 95%, at least 99%, or 100% identical to SEQ ID NO: 21 or 22.

In some aspects, the first polypeptide comprises a structure represented by VL1-VL2-CH1; VL1-L1-VL2-L2-CH1; VL1-VL2-CH1-CH2-CH3; or VL1-L1-VL2-L2-CH1-CH2-CH3 and the second polypeptide comprises a structure represented by VH1-VH2-CL; VH1-L3-VH2-L4-CL; VH1-VH2-CL-CH2-CH3; or VH1-L3-VH2-L4-CL-CH2-CH3.

In some aspects, the first polypeptide comprises a structure represented by VL1-VL2-VL3-CH1; VL1-L1-VL2-L2-VL3-L3-CH1; VL1-VL2-VL3-CH1-CH2-CH3; or VL1-L1-VL2-L2-VL3-L3-CH1-CH2-CH3 and the second polypeptide comprises a structure represented by VH1-VH2-VH3-CL; VH1-L4-VH2-L5-VH3-L6-CL; or VH1-L4-VH2-L5-VH3-L6-CL-CH2-CH3.

In some aspects, the first polypeptide comprises a structure represented by VL1-VH1-CL, VL1-L1-VH1-L2-CL; VL1-VH1-CL-CH2-CH3; VL1-L1-VH1-L2-CL-CH2-CH3 and the second polypeptide comprises a structure represented by VL2-VH2-CH1; VL2-L3-VH2-L4-CH1; VL2-VH2-CH1-CH2-CH3; VL2-L3-VH2-L4-CH1-CH2-CH3;
wherein:
(a) VL1 comprises an amino acid sequence at least 65%, at least 70%, at least 75%, at least 80%, at least 85%, at least 90%, at least 95%, at least 99%, or 100% identical to SEQ ID NO: 17, VH1 comprises an amino acid sequence at least 65%, at least 70%, at least 75%, at least 80%, at least 85%, at least 90%, at least 95%, at least 99%, or 100% identical to SEQ ID NO: 21 or 22, VL2 comprises an amino acid sequence at least 65%, at least 70%, at least 75%, at least 80%, at least 85%, at least 90%, at least 95%, at least 99%, or 100% identical to SEQ ID NO: 17, and VH2 comprises an amino acid sequence at least 65%, at least 70%, at least 75%, at least 80%, at least 85%, at least 90%, at least 95%, at least 99%, or 100% identical to SEQ ID NO: 21 or 22; or
(b) VL1 comprises an amino acid sequence at least 65%, at least 70%, at least 75%, at least 80%, at least 85%, at least 90%, at least 95%, at least 99%, or 100% identical to SEQ ID NO: 11, VH1 comprises an amino acid sequence at least 65%, at least 70%, at least 75%, at least 80%, at least 85%, at least 90%, at least 95%, at least 99%, or 100% identical to SEQ ID NO: 14, VL2 comprises an amino acid sequence at least 65%, at least 70%, at least 75%, at least 80%, at least 85%, at least 90%, at least 95%, at least 99%, or 100% identical to SEQ ID NO: 11, and VH2 comprises an amino acid sequence at least 65%, at least 70%, at least 75%, at least 80%, at least 85%, at least 90%, at least 95%, at least 99%, or 100% identical to SEQ ID NO: 14; or
(c) VL1 comprises an amino acid sequence at least 65%, at least 70%, at least 75%, at least 80%, at least 85%, at least 90%, at least 95%, at least 99%, or 100% identical to SEQ ID NO: 74, VH1 comprises an amino acid sequence at least 65%, at least 70%, at least 75%, at least 80%, at least 85%, at least 90%, at least 95%, at least 99%, or 100% identical to SEQ ID NO: 78, VL2 comprises an amino acid sequence at least 65%, at least 70%, at least 75%, at least 80%, at least 85%, at least 90%, at least 95%, at least 99%, or 100% identical to SEQ ID NO: 74, and VH2 comprises an amino acid sequence at least 65%, at least 70%, at least 75%, at least 80%, at least 85%, at least 90%, at least 95%, at least 99%, or 100% identical to SEQ ID NO: 78.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOS: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, an antigen binding polypeptide complex provided herein comprises a first polypeptide, and a second polypeptide; wherein the first polypeptide has a structure represented by:
VL1-L1-VL2-L2-VH2-L3-VH1-L4-Fc;
VL1-L1-VH2-L2-VL2-L3-VH1-L4-Fc;
VH1-L1-VH2-L2-VL2-L3-VL1-L4-Fc; or
VH1-L1-VL2-L2-VH2-L3-VL1-L4-Fc;
   and wherein the second polypeptide has a structure represented by:
VL3-L5-VL4-L6-VH4-L7-VH3-L8-Fc;
VL3-L5-VH4-L6-VL4-L7-VH3-L8-Fc;
VH3-L5-VH4-L6-VL4-L7-VL3-L8-Fc; or
VH3-L5-VL4-L6-VH4-L7-VL3-L8-Fc;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VL3 is a third immunoglobulin light chain variable region that specifically binds to a SARS-CoV-2 protein;
VL4 is a fourth immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
VH4 is a fourth immunoglobulin heavy chain variable region that specifically binds to a SARS-CoV-2 protein;
L1-L8 are optional amino acid linkers; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In some aspects, an antigen binding polypeptide complex provided herein comprises a first polypeptide, and a second polypeptide; wherein the first polypeptide has a structure represented by:
VL1-L1-VL2-L2-VH2-L3-VH1-L4-CH1-L5-CL-L6-Fc;
VL1-L1-VH2-L2-VL2-L3-VH1-L4-CH1-L5-CL-L6-Fc;
VH1-L1-VH2-L2-VL2-L3-VL1-L4-CH1-L5-CL-L6-Fc;
VH1-L1-VL2-L2-VH2-L3-VL1-L4-CH1-L5-CL-L6-Fc;
VL1-L1-VL2-L2-VH2-L3-VH1-L4-CL-L5-CH1-L6-Fc;
VL1-L1-VH2-L2-VL2-L3-VH1-L4-CL-L5-CH1-L6-Fc;
VH1-L1-VH2-L2-VL2-L3-VL1-L4-CL-L5-CH1-L6-Fc; or
VH1-L1-VL2-L2-VH2-L3-VL1-L4-CL-L5-CH1-L6-Fc;
   and wherein the second polypeptide has a structure represented by:
VL3-L7-VL4-L8-VH4-L9-VH3-L10-CH1-L11-CL-L12-Fc;
VL3-L7-VH4-L8-VL4-L9-VH3-L10-CH1-L11-CL-L12-Fc;
VH3-L7-VH4-L8-VL4-L9-VL3-L10-CH1-L11-CL-L12-Fc;
VH3-L7-VL4-L8-VH4-L9-VL3-L10-CH1-L11-CL-L12-Fc;
VL3-L7-VL4-L8-VH4-L9-VH3-L10-CL-L11-CH1-L12-Fc;
VL3-L7-VH4-L8-VL4-L9-VH3-L10-CL-L11-CH1-L12-Fc;
VH3-L7-VH4-L8-VL4-L9-VL3-L10-CL-L11-CH1-L12-Fc; or
VH3-L7-VL4-L8-VH4-L9-VL3-L10-CL-L11-CH1-L12-Fc;
   wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VL3 is a third immunoglobulin light chain variable region that specifically binds to a SARS-CoV-2 protein;
VL4 is a fourth immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
VH4 is a fourth immunoglobulin heavy chain variable region that specifically binds to a SARS-CoV-2 protein;
L1-L12 are optional amino acid linkers;
CH1 is an immunoglobulin heavy chain constant region 1;
CL is an immunoglobulin light chain constant region; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VH3 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VH4 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; the VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; the VH3 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; the VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and the VH4 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL3 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the VH3 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL4 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the VH4 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (v) the VL3 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (vi) the VH3 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (vii) the VL4 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (viii) the VH4 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VH3 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VH4 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; the VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; the VH3 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; the VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31.628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and the VH4 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the VL3 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the VH3 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the VL4 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the VH4 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (v) the VL3 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (vi) the VH3 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178. (vii) the VL4 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (viii) the VH4 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VH3 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VH4 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; the VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; the VH3 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; the VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; and the VH4 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the VL3 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the VH3 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the VL4 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the VH4 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; (v) the VL3 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; (vi) the VH3 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; (vii) the VL4 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; and (viii) the VH4 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, at least one of VL1, VL2, VL3, or VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1, VH2, VH3, or VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1, VL2, VL3, or VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1, VH2, VH3, or VH4 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1, VL2, VL3, or VL4 comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1, VH2, VH3, or VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1, VL2, VL3, or VL4 comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1, VH2, VH3, or VH4 comprises an amino acid sequence that is at least 65%, at least 70%, at least 75%, at least 80%, at least 85%, at least 90%, at least 95%, at least 99%, or 100% identical to SEQ ID NO: 151.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; the VH1 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; the VL2 comprises (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; the VH2 comprises (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; the VL3 comprises (xiii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (xiv) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (xv) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; the VH3 comprises (xvi) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xvii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; (xviii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; the VL4 comprises (xix) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (xx) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (xxi) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and the VH4 comprises (xxii) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xxiii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; (xxiv) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (i) the VL1 comprises an amino acid sequence that is at least 65%, at least 70%, at least 75%, at least 80%, at least 85%, at least 90%, at least 95%, at least 99%, or 100% identical to SEQ ID NO: 147; (ii) the VH1 comprises an amino acid sequence that is at least 65%, at least 70%, at least 75%, at least 80%, at least 85%, at least 90%, at least 95%, at least 99%, or 100% identical to SEQ ID NO: 151; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (v) the VL3 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (vi) the VH3 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (vii) the VL4 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (viii) the VH4 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; the VH1 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; the VL2 comprises (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; the VH2 comprises (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; the VL3 comprises (xiii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (xiv) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (xv) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; the VH3 comprises (xvi) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xvii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xviii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; the VL4 comprises (xix) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (xx) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (xxi) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and the VH4 comprises (xxii) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xxiii) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xxiv) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, (i) the VL1 comprises an amino acid sequence that is at least 65%, at least 70%, at least 75%, at least 80%, at least 85%, at least 90%, at least 95%, at least 99%, or 100% identical to SEQ ID NO: 147; (ii) the VH1 comprises an amino acid sequence that is at least 65%, at least 70%, at least 75%, at least 80%, at least 85%, at least 90%, at least 95%, at least 99%, or 100% identical to SEQ ID NO: 151; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (v) the VL3 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (vi) the VH3 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (vii) the VL4 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (viii) the VH4 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof. In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, an antigen binding polypeptide complex provided herein comprises two antigen binding polypeptides wherein at least one antigen binding polypeptide has a structure represented by:
VL1-L1-VL2-L2-VH2-L3-VH1-L4-Fc-L5-Fc;
VL1-L1-VH2-L2-VL2-L3-VH1-L4-Fc-L5-Fc;
VH1-L1-VH2-L2-VL2-L3-VL1-L4-Fc-L5-Fc; or
VH1-L1-VL2-L2-VH2-L3-VL1-L4-Fc-L5-Fc;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
L1-L5 are optional amino acid linkers; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865.878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324.627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; and the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, at least one of VL1 or VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2, comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1 or VH2 comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprises an amino acid sequence that is at least 65%, at least 70%, at least 75%, at least 80%, at least 85%, at least 90%, at least 95%, at least 99%, or 100% identical to SEQ ID NO: 151.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; the VH1 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; the VL2 comprises (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and the VH2 comprises (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (i) the VL1 comprises an amino acid sequence that is at least 65%, at least 70%, at least 75%, at least 80%, at least 85%, at least 90%, at least 95%, at least 99%, or 100% identical to SEQ ID NO: 147; (ii) the VH1 comprises an amino acid sequence that is at least 65%, at least 70%, at least 75%, at least 80%, at least 85%, at least 90%, at least 95%, at least 99%, or 100% identical to SEQ ID NO: 151; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; the VH1 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; the VL2 comprises (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and the VH2 comprises (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, (i) the VL1 comprises an amino acid sequence that is at least 65%, at least 70%, at least 75%, at least 80%, at least 85%, at least 90%, at least 95%, at least 99%, or 100% identical to SEQ ID NO: 147; (ii) the VH1 comprises an amino acid sequence that is at least 65%, at least 70%, at least 75%, at least 80%, at least 85%, at least 90%, at least 95%, at least 99%, or 100% identical to SEQ ID NO: 151; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOS: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635.636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOS: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the first polypeptide and the second polypeptide comprise a same amino acid sequence.

In some aspects, the first polypeptide and the second polypeptide comprise a different amino acid sequence.

In some aspects, the first polypeptide and the second polypeptide are encoded by the same nucleotide sequence.

In some aspects, the first polypeptide and the second polypeptide are encoded by a different nucleotide sequence.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 381 and the second polypeptide comprises an amino acid sequence at least 65%, at least 70%, at least 75%, at least 80%, at least 85%, at least 90%, at least 95%, at least 99%, or 100% identical to SEQ ID NO: 381.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 382 and the second polypeptide comprises an amino acid sequence at least 65%, at least 70%, at least 75%, at least 80%, at least 85%, at least 90%, at least 95%, at least 99%, or 100% identical to SEQ ID NO: 382.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 383 and the second polypeptide comprises an amino acid sequence at least 65%, at least 70%, at least 75%, at least 80%, at least 85%, at least 90%, at least 95%, at least 99%, or 100% identical to SEQ ID NO: 383.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 384 and the second polypeptide comprises an amino acid sequence at least 65%, at least 70%, at least 75%, at least 80%, at least 85%, at least 90%, at least 95%, at least 99%, or 100% identical to SEQ ID NO: 385.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 386 and the second polypeptide comprises an amino acid sequence at least 65%, at least 70%, at least 75%, at least 80%, at least 85%, at least 90%, at least 95%, at least 99%, or 100% identical to SEQ ID NO: 387.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 388 and the second polypeptide comprises an amino acid sequence at least 65%, at least 70%, at least 75%, at least 80%, at least 85%, at least 90%, at least 95%, at least 99%, or 100% identical to SEQ ID NO: 388.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 389 and the second polypeptide comprises an amino acid sequence at least 65%, at least 70%, at least 75%, at least 80%, at least 85%, at least 90%, at least 95%, at least 99%, or 100% identical to SEQ ID NO: 389.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 390 and the second polypeptide comprises an amino acid sequence at least 65%, at least 70%, at least 75%, at least 80%, at least 85%, at least 90%, at least 95%, at least 99%, or 100% identical to SEQ ID NO: 390.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 391 and the second polypeptide comprises an amino acid sequence at least 65%, at least 70%, at least 75%, at least 80%, at least 85%, at least 90%, at least 95%, at least 99%, or 100% identical to SEQ ID NO: 391.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 392 and the second polypeptide comprises an amino acid sequence at least 65%, at least 70%, at least 75%, at least 80%, at least 85%, at least 90%, at least 95%, at least 99%, or 100% identical to SEQ ID NO: 393.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 394 and the second polypeptide comprises an amino acid sequence at least 65%, at least 70%, at least 75%, at least 80%, at least 85%, at least 90%, at least 95%, at least 99%, or 100% identical to SEQ ID NO: 395.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 396 and the second polypeptide comprises an amino acid sequence at least 65%, at least 70%, at least 75%, at least 80%, at least 85%, at least 90%, at least 95%, at least 99%, or 100% identical to SEQ ID NO: 397.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 398 and the second polypeptide comprises an amino acid sequence at least 65%, at least 70%, at least 75%, at least 80%, at least 85%, at least 90%, at least 95%, at least 99%, or 100% identical to SEQ ID NO: 399.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 400 and the second polypeptide comprises an amino acid sequence at least 65%, at least 70%, at least 75%, at least 80%, at least 85%, at least 90%, at least 95%, at least 99%, or 100% identical to SEQ ID NO: 400.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 401 and the second polypeptide comprises an amino acid sequence at least 65%, at least 70%, at least 75%, at least 80%, at least 85%, at least 90%, at least 95%, at least 99%, or 100% identical to SEQ ID NO: 401.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 402 and the second polypeptide comprises an amino acid sequence at least 65%, at least 70%, at least 75%, at least 80%, at least 85%, at least 90%, at least 95%, at least 99%, or 100% identical to SEQ ID NO: 402.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 403 and the second polypeptide comprises an amino acid sequence at least 65%, at least 70%, at least 75%, at least 80%, at least 85%, at least 90%, at least 95%, at least 99%, or 100% identical to SEQ ID NO: 403.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 404 and the second polypeptide comprises an amino acid sequence at least 65%, at least 70%, at least 75%, at least 80%, at least 85%, at least 90%, at least 95%, at least 99%, or 100% identical to SEQ ID NO: 404.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 405 and the second polypeptide comprises an amino acid sequence at least 65%, at least 70%, at least 75%, at least 80%, at least 85%, at least 90%, at least 95%, at least 99%, or 100% identical to SEQ ID NO: 406.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 407 and the second polypeptide comprises an amino acid sequence at least 65%, at least 70%, at least 75%, at least 80%, at least 85%, at least 90%, at least 95%, at least 99%, or 100% identical to SEQ ID NO: 408.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 409 and the second polypeptide comprises an amino acid sequence at least 65%, at least 70%, at least 75%, at least 80%, at least 85%, at least 90%, at least 95%, at least 99%, or 100% identical to SEQ ID NO: 410.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 411 and the second polypeptide comprises an amino acid sequence at least 65%, at least 70%, at least 75%, at least 80%, at least 85%, at least 90%, at least 95%, at least 99%, or 100% identical to SEQ ID NO: 412.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 413 and the second polypeptide comprises an amino acid sequence at least 65%, at least 70%, at least 75%, at least 80%, at least 85%, at least 90%, at least 95%, at least 99%, or 100% identical to SEQ ID NO: 414.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 415 and the second polypeptide comprises an amino acid sequence at least 65%, at least 70%, at least 75%, at least 80%, at least 85%, at least 90%, at least 95%, at least 99%, or 100% identical to SEQ ID NO: 416.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 417 and the second polypeptide comprises an amino acid sequence at least 65%, at least 70%, at least 75%, at least 80%, at least 85%, at least 90%, at least 95%, at least 99%, or 100% identical to SEQ ID NO: 418.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 419 and the second polypeptide comprises an amino acid sequence at least 65%, at least 70%, at least 75%, at least 80%, at least 85%, at least 90%, at least 95%, at least 99%, or 100% identical to SEQ ID NO: 420.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 421 and the second polypeptide comprises an amino acid sequence at least 65%, at least 70%, at least 75%, at least 80%, at least 85%, at least 90%, at least 95%, at least 99%, or 100% identical to SEQ ID NO: 422.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 423 and the second polypeptide comprises an amino acid sequence at least 65%, at least 70%, at least 75%, at least 80%, at least 85%, at least 90%, at least 95%, at least 99%, or 100% identical to SEQ ID NO: 424.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 425 and the second polypeptide comprises an amino acid sequence at least 65%, at least 70%, at least 75%, at least 80%, at least 85%, at least 90%, at least 95%, at least 99%, or 100% identical to SEQ ID NO: 426.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 427 and the second polypeptide comprises an amino acid sequence at least 65%, at least 70%, at least 75%, at least 80%, at least 85%, at least 90%, at least 95%, at least 99%, or 100% identical to SEQ ID NO: 428.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 429 and the second polypeptide comprises an amino acid sequence at least 65%, at least 70%, at least 75%, at least 80%, at least 85%, at least 90%, at least 95%, at least 99%, or 100% identical to SEQ ID NO: 430.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 431 and the second polypeptide comprises an amino acid sequence at least 65%, at least 70%, at least 75%, at least 80%, at least 85%, at least 90%, at least 95%, at least 99%, or 100% identical to SEQ ID NO: 432.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 433 and the second polypeptide comprises an amino acid sequence at least 65%, at least 70%, at least 75%, at least 80%, at least 85%, at least 90%, at least 95%, at least 99%, or 100% identical to SEQ ID NO: 434.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 435 and the second polypeptide comprises an amino acid sequence at least 65%, at least 70%, at least 75%, at least 80%, at least 85%, at least 90%, at least 95%, at least 99%, or 100% identical to SEQ ID NO: 436.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 437 and the second polypeptide comprises an amino acid sequence at least 65%, at least 70%, at least 75%, at least 80%, at least 85%, at least 90%, at least 95%, at least 99%, or 100% identical to SEQ ID NO: 438.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 439 and the second polypeptide comprises an amino acid sequence at least 65%, at least 70%, at least 75%, at least 80%, at least 85%, at least 90%, at least 95%, at least 99%, or 100% identical to SEQ ID NO: 440.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 441 and the second polypeptide comprises an amino acid sequence at least 65%, at least 70%, at least 75%, at least 80%, at least 85%, at least 90%, at least 95%, at least 99%, or 100% identical to SEQ ID NO: 442.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 443 and the second polypeptide comprises an amino acid sequence at least 65%, at least 70%, at least 75%, at least 80%, at least 85%, at least 90%, at least 95%, at least 99%, or 100% identical to SEQ ID NO: 444.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 445 and the second polypeptide comprises an amino acid sequence at least 65%, at least 70%, at least 75%, at least 80%, at least 85%, at least 90%, at least 95%, at least 99%, or 100% identical to SEQ ID NO: 446.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 447 and the second polypeptide comprises an amino acid sequence at least 65%, at least 70%, at least 75%, at least 80%, at least 85%, at least 90%, at least 95%, at least 99%, or 100% identical to SEQ ID NO: 448.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 449 and the second polypeptide comprises an amino acid sequence at least 65%, at least 70%, at least 75%, at least 80%, at least 85%, at least 90%, at least 95%, at least 99%, or 100% identical to SEQ ID NO: 450.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 451 and the second polypeptide comprises an amino acid sequence at least 65%, at least 70%, at least 75%, at least 80%, at least 85%, at least 90%, at least 95%, at least 99%, or 100% identical to SEQ ID NO: 452.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 453 and the second polypeptide comprises an amino acid sequence at least 65%, at least 70%, at least 75%, at least 80%, at least 85%, at least 90%, at least 95%, at least 99%, or 100% identical to SEQ ID NO: 454.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 455 and the second polypeptide comprises an amino acid sequence at least 65%, at least 70%, at least 75%, at least 80%, at least 85%, at least 90%, at least 95%, at least 99%, or 100% identical to SEQ ID NO: 456.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 457 and the second polypeptide comprises an amino acid sequence at least 65%, at least 70%, at least 75%, at least 80%, at least 85%, at least 90%, at least 95%, at least 99%, or 100% identical to SEQ ID NO: 458.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 459 and the second polypeptide comprises an amino acid sequence at least 65%, at least 70%, at least 75%, at least 80%, at least 85%, at least 90%, at least 95%, at least 99%, or 100% identical to SEQ ID NO: 460.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 461 and the second polypeptide comprises an amino acid sequence at least 65%, at least 70%, at least 75%, at least 80%, at least 85%, at least 90%, at least 95%, at least 99%, or 100% identical to SEQ ID NO: 462.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 463 and the second polypeptide comprises an amino acid sequence at least 65%, at least 70%, at least 75%, at least 80%, at least 85%, at least 90%, at least 95%, at least 99%, or 100% identical to SEQ ID NO: 464.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 465 and the second polypeptide comprises an amino acid sequence at least 65%, at least 70%, at least 75%, at least 80%, at least 85%, at least 90%, at least 95%, at least 99%, or 100% identical to SEQ ID NO: 466.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 467 and the second polypeptide comprises an amino acid sequence at least 65%, at least 70%, at least 75%, at least 80%, at least 85%, at least 90%, at least 95%, at least 99%, or 100% identical to SEQ ID NO: 468.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 469 and the second polypeptide comprises an amino acid sequence at least 65%, at least 70%, at least 75%, at least 80%, at least 85%, at least 90%, at least 95%, at least 99%, or 100% identical to SEQ ID NO: 469.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 470 and the second polypeptide comprises an amino acid sequence at least 65%, at least 70%, at least 75%, at least 80%, at least 85%, at least 90%, at least 95%, at least 99%, or 100% identical to SEQ ID NO: 470.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 471 and the second polypeptide comprises an amino acid sequence at least 65%, at least 70%, at least 75%, at least 80%, at least 85%, at least 90%, at least 95%, at least 99%, or 100% identical to SEQ ID NO: 471.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 472 and the second polypeptide comprises an amino acid sequence at least 65%, at least 70%, at least 75%, at least 80%, at least 85%, at least 90%, at least 95%, at least 99%, or 100% identical to SEQ ID NO: 473.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 474 and the second polypeptide comprises an amino acid sequence at least 65%, at least 70%, at least 75%, at least 80%, at least 85%, at least 90%, at least 95%, at least 99%, or 100% identical to SEQ ID NO: 475.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 476 and the second polypeptide comprises an amino acid sequence at least 65%, at least 70%, at least 75%, at least 80%, at least 85%, at least 90%, at least 95%, at least 99%, or 100% identical to SEQ ID NO: 477.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 478 and the second polypeptide comprises an amino acid sequence at least 65%, at least 70%, at least 75%, at least 80%, at least 85%, at least 90%, at least 95%, at least 99%, or 100% identical to SEQ ID NO: 478.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 479 and the second polypeptide comprises an amino acid sequence at least 65%, at least 70%, at least 75%, at least 80%, at least 85%, at least 90%, at least 95%, at least 99%, or 100% identical to SEQ ID NO: 479.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 480 and the second polypeptide comprises an amino acid sequence at least 65%, at least 70%, at least 75%, at least 80%, at least 85%, at least 90%, at least 95%, at least 99%, or 100% identical to SEQ ID NO: 480.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 481 and the second polypeptide comprises an amino acid sequence at least 65%, at least 70%, at least 75%, at least 80%, at least 85%, at least 90%, at least 95%, at least 99%, or 100% identical to SEQ ID NO: 481.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 482 and the second polypeptide comprises an amino acid sequence at least 65%, at least 70%, at least 75%, at least 80%, at least 85%, at least 90%, at least 95%, at least 99%, or 100% identical to SEQ ID NO: 482.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 483 and the second polypeptide comprises an amino acid sequence at least 65%, at least 70%, at least 75%, at least 80%, at least 85%, at least 90%, at least 95%, at least 99%, or 100% identical to SEQ ID NO: 483.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 484 and the second polypeptide comprises an amino acid sequence at least 65%, at least 70%, at least 75%, at least 80%, at least 85%, at least 90%, at least 95%, at least 99%, or 100% identical to SEQ ID NO: 484.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 485 and the second polypeptide comprises an amino acid sequence at least 65%, at least 70%, at least 75%, at least 80%, at least 85%, at least 90%, at least 95%, at least 99%, or 100% identical to SEQ ID NO: 486.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 487 and the second polypeptide comprises an amino acid sequence at least 65%, at least 70%, at least 75%, at least 80%, at least 85%, at least 90%, at least 95%, at least 99%, or 100% identical to SEQ ID NO: 488.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 489 and the second polypeptide comprises an amino acid sequence at least 65%, at least 70%, at least 75%, at least 80%, at least 85%, at least 90%, at least 95%, at least 99%, or 100% identical to SEQ ID NO: 490.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 491 and the second polypeptide comprises an amino acid sequence at least 65%, at least 70%, at least 75%, at least 80%, at least 85%, at least 90%, at least 95%, at least 99%, or 100% identical to SEQ ID NO: 492.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 493 and the second polypeptide comprises an amino acid sequence at least 65%, at least 70%, at least 75%, at least 80%, at least 85%, at least 90%, at least 95%, at least 99%, or 100% identical to SEQ ID NO: 494.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 495 and the second polypeptide comprises an amino acid sequence at least 65%, at least 70%, at least 75%, at least 80%, at least 85%, at least 90%, at least 95%, at least 99%, or 100% identical to SEQ ID NO: 496.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 497 and the second polypeptide comprises an amino acid sequence at least 65%, at least 70%, at least 75%, at least 80%, at least 85%, at least 90%, at least 95%, at least 99%, or 100% identical to SEQ ID NO: 498.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 499 and the second polypeptide comprises an amino acid sequence at least 65%, at least 70%, at least 75%, at least 80%, at least 85%, at least 90%, at least 95%, at least 99%, or 100% identical to SEQ ID NO: 500.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 501 and the second polypeptide comprises an amino acid sequence at least 65%, at least 70%, at least 75%, at least 80%, at least 85%, at least 90%, at least 95%, at least 99%, or 100% identical to SEQ ID NO: 502.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 632 and the second polypeptide comprises an amino acid sequence at least 65%, at least 70%, at least 75%, at least 80%, at least 85%, at least 90%, at least 95%, at least 99%, or 100% identical to SEQ ID NO: 633.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 720 and the second polypeptide comprises an amino acid sequence at least 65%, at least 70%, at least 75%, at least 80%, at least 85%, at least 90%, at least 95%, at least 99%, or 100% identical to SEQ ID NO: 721.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 724 and the second polypeptide comprises an amino acid sequence at least 65%, at least 70%, at least 75%, at least 80%, at least 85%, at least 90%, at least 95%, at least 99%, or 100% identical to SEQ ID NO: 725.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 728 and the second polypeptide comprises an amino acid sequence at least 65%, at least 70%, at least 75%, at least 80%, at least 85%, at least 90%, at least 95%, at least 99%, or 100% identical to SEQ ID NO: 729.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 732 and the second polypeptide comprises an amino acid sequence at least 65%, at least 70%, at least 75%, at least 80%, at least 85%, at least 90%, at least 95%, at least 99%, or 100% identical to SEQ ID NO: 733.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 736 and the second polypeptide comprises an amino acid sequence at least 65%, at least 70%, at least 75%, at least 80%, at least 85%, at least 90%, at least 95%, at least 99%, or 100% identical to SEQ ID NO: 737.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 740 and the second polypeptide comprises an amino acid sequence at least 65%, at least 70%, at least 75%, at least 80%, at least 85%, at least 90%, at least 95%, at least 99%, or 100% identical to SEQ ID NO: 741.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 744 and the second polypeptide comprises an amino acid sequence at least 65%, at least 70%, at least 75%, at least 80%, at least 85%, at least 90%, at least 95%, at least 99%, or 100% identical to SEQ ID NO: 745.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 748 and the second polypeptide comprises an amino acid sequence at least 65%, at least 70%, at least 75%, at least 80%, at least 85%, at least 90%, at least 95%, at least 99%, or 100% identical to SEQ ID NO: 749.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 752 and the second polypeptide comprises an amino acid sequence at least 65%, at least 70%, at least 75%, at least 80%, at least 85%, at least 90%, at least 95%, at least 99%, or 100% identical to SEQ ID NO: 753.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 760 and the second polypeptide comprises an amino acid sequence at least 65%, at least 70%, at least 75%, at least 80%, at least 85%, at least 90%, at least 95%, at least 99%, or 100% identical to SEQ ID NO: 761.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 887 and the second polypeptide comprises an amino acid sequence at least 65%, at least 70%, at least 75%, at least 80%, at least 85%, at least 90%, at least 95%, at least 99%, or 100% identical to SEQ ID NO: 888.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 889 and the second polypeptide comprises an amino acid sequence at least 65%, at least 70%, at least 75%, at least 80%, at least 85%, at least 90%, at least 95%, at least 99%, or 100% identical to SEQ ID NO: 900.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 891 and the second polypeptide comprises an amino acid sequence at least 65%, at least 70%, at least 75%, at least 80%, at least 85%, at least 90%, at least 95%, at least 99%, or 100% identical to SEQ ID NO: 892.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 893 and the second polypeptide comprises an amino acid sequence at least 65%, at least 70%, at least 75%, at least 80%, at least 85%, at least 90%, at least 95%, at least 99%, or 100% identical to SEQ ID NO: 894.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 895 and the second polypeptide comprises an amino acid sequence at least 65%, at least 70%, at least 75%, at least 80%, at least 85%, at least 90%, at least 95%, at least 99%, or 100% identical to SEQ ID NO: 896.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 897 and the second polypeptide comprises an amino acid sequence at least 65%, at least 70%, at least 75%, at least 80%, at least 85%, at least 90%, at least 95%, at least 99%, or 100% identical to SEQ ID NO: 898.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 899 and the second polypeptide comprises an amino acid sequence at least 65%, at least 70%, at least 75%, at least 80%, at least 85%, at least 90%, at least 95%, at least 99%, or 100% identical to SEQ ID NO: 900.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 901 and the second polypeptide comprises an amino acid sequence at least 65%, at least 70%, at least 75%, at least 80%, at least 85%, at least 90%, at least 95%, at least 99%, or 100% identical to SEQ ID NO: 902.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 903 and the second polypeptide comprises an amino acid sequence at least 65%, at least 70%, at least 75%, at least 80%, at least 85%, at least 90%, at least 95%, at least 99%, or 100% identical to SEQ ID NO: 904.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 905 and the second polypeptide comprises an amino acid sequence at least 65%, at least 70%, at least 75%, at least 80%, at least 85%, at least 90%, at least 95%, at least 99%, or 100% identical to SEQ ID NO: 906.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 907 and the second polypeptide comprises an amino acid sequence at least 65%, at least 70%, at least 75%, at least 80%, at least 85%, at least 90%, at least 95%, at least 99%, or 100% identical to SEQ ID NO: 908.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 909 and the second polypeptide comprises an amino acid sequence at least 65%, at least 70%, at least 75%, at least 80%, at least 85%, at least 90%, at least 95%, at least 99%, or 100% identical to SEQ ID NO: 910.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 911 and the second polypeptide comprises an amino acid sequence at least 65%, at least 70%, at least 75%, at least 80%, at least 85%, at least 90%, at least 95%, at least 99%, or 100% identical to SEQ ID NO: 912.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 913 and the second polypeptide comprises an amino acid sequence at least 65%, at least 70%, at least 75%, at least 80%, at least 85%, at least 90%, at least 95%, at least 99%, or 100% identical to SEQ ID NO: 914.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 915 and the second polypeptide comprises an amino acid sequence at least 65%, at least 70%, at least 75%, at least 80%, at least 85%, at least 90%, at least 95%, at least 99%, or 100% identical to SEQ ID NO: 916.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 917 and the second polypeptide comprises an amino acid sequence at least 65%, at least 70%, at least 75%, at least 80%, at least 85%, at least 90%, at least 95%, at least 99%, or 100% identical to SEQ ID NO: 918.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 919 and the second polypeptide comprises an amino acid sequence at least 65%, at least 70%, at least 75%, at least 80%, at least 85%, at least 90%, at least 95%, at least 99%, or 100% identical to SEQ ID NO: 920.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 921 and the second polypeptide comprises an amino acid sequence at least 65%, at least 70%, at least 75%, at least 80%, at least 85%, at least 90%, at least 95%, at least 99%, or 100% identical to SEQ ID NO: 922.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 923 and the second polypeptide comprises an amino acid sequence at least 65%, at least 70%, at least 75%, at least 80%, at least 85%, at least 90%, at least 95%, at least 99%, or 100% identical to SEQ ID NO: 924.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 925 and the second polypeptide comprises an amino acid sequence at least 65%, at least 70%, at least 75%, at least 80%, at least 85%, at least 90%, at least 95%, at least 99%, or 100% identical to SEQ ID NO: 926.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 972 and the second polypeptide comprises an amino acid sequence at least 65%, at least 70%, at least 75%, at least 80%, at least 85%, at least 90%, at least 95%, at least 99%, or 100% identical to SEQ ID NO: 972.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 973 and the second polypeptide comprises an amino acid sequence at least 65%, at least 70%, at least 75%, at least 80%, at least 85%, at least 90%, at least 95%, at least 99%, or 100% identical to SEQ ID NO: 973.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 974 and the second polypeptide comprises an amino acid sequence at least 65%, at least 70%, at least 75%, at least 80%, at least 85%, at least 90%, at least 95%, at least 99%, or 100% identical to SEQ ID NO: 974.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 975 and the second polypeptide comprises an amino acid sequence at least 65%, at least 70%, at least 75%, at least 80%, at least 85%, at least 90%, at least 95%, at least 99%, or 100% identical to SEQ ID NO: 975.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 976 and the second polypeptide comprises an amino acid sequence at least 65%, at least 70%, at least 75%, at least 80%, at least 85%, at least 90%, at least 95%, at least 99%, or 100% identical to SEQ ID NO: 976.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 977 and the second polypeptide comprises an amino acid sequence at least 65%, at least 70%, at least 75%, at least 80%, at least 85%, at least 90%, at least 95%, at least 99%, or 100% identical to SEQ ID NO: 977.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 984 and the second polypeptide comprises an amino acid sequence at least 65%, at least 70%, at least 75%, at least 80%, at least 85%, at least 90%, at least 95%, at least 99%, or 100% identical to SEQ ID NO: 985.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 989 and the second polypeptide comprises an amino acid sequence at least 65%, at least 70%, at least 75%, at least 80%, at least 85%, at least 90%, at least 95%, at least 99%, or 100% identical to SEQ ID NO: 990.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 993 and the second polypeptide comprises an amino acid sequence at least 65%, at least 70%, at least 75%, at least 80%, at least 85%, at least 90%, at least 95%, at least 99%, or 100% identical to SEQ ID NO: 994.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 997 and the second polypeptide comprises an amino acid sequence at least 65%, at least 70%, at least 75%, at least 80%, at least 85%, at least 90%, at least 95%, at least 99%, or 100% identical to SEQ ID NO: 998.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1001 and the second polypeptide comprises an amino acid sequence at least 65%, at least 70%, at least 75%, at least 80%, at least 85%, at least 90%, at least 95%, at least 99%, or 100% identical to SEQ ID NO: 1002.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1005 and the second polypeptide comprises an amino acid sequence at least 65%, at least 70%, at least 75%, at least 80%, at least 85%, at least 90%, at least 95%, at least 99%, or 100% identical to SEQ ID NO: 1006.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1009 and the second polypeptide comprises an amino acid sequence at least 65%, at least 70%, at least 75%, at least 80%, at least 85%, at least 90%, at least 95%, at least 99%, or 100% identical to SEQ ID NO: 1010.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1013 and the second polypeptide comprises an amino acid sequence at least 65%, at least 70%, at least 75%, at least 80%, at least 85%, at least 90%, at least 95%, at least 99%, or 100% identical to SEQ ID NO: 1014.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1017 and the second polypeptide comprises an amino acid sequence at least 65%, at least 70%, at least 75%, at least 80%, at least 85%, at least 90%, at least 95%, at least 99%, or 100% identical to SEQ ID NO: 1018.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1021 and the second polypeptide comprises an amino acid sequence at least 65%, at least 70%, at least 75%, at least 80%, at least 85%, at least 90%, at least 95%, at least 99%, or 100% identical to SEQ ID NO: 1022.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1025 and the second polypeptide comprises an amino acid sequence at least 65%, at least 70%, at least 75%, at least 80%, at least 85%, at least 90%, at least 95%, at least 99%, or 100% identical to SEQ ID NO: 1026.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1029 and the second polypeptide comprises an amino acid sequence at least 65%, at least 70%, at least 75%, at least 80%, at least 85%, at least 90%, at least 95%, at least 99%, or 100% identical to SEQ ID NO: 1030.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1033 and the second polypeptide comprises an amino acid sequence at least 65%, at least 70%, at least 75%, at least 80%, at least 85%, at least 90%, at least 95%, at least 99%, or 100% identical to SEQ ID NO: 1034.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1037 and the second polypeptide comprises an amino acid sequence at least 65%, at least 70%, at least 75%, at least 80%, at least 85%, at least 90%, at least 95%, at least 99%, or 100% identical to SEQ ID NO: 1038.

In some aspects, the first polypeptide comprises an amino acid sequence at least 65%, at least 70%, at least 75%, at least 80%, at least 85%, at least 90%, at least 95%, at least 99%, or 100% identical to SEQ ID NO: 1041 and the second polypeptide comprises an amino acid sequence at least 65%, at least 70%, at least 75%, at least 80%, at least 85%, at least 90%, at least 95%, at least 99%, or 100% identical to SEQ ID NO: 1042.

In some aspects, the antigen binding polypeptides or the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides or the antigen binding polypeptide complexes disclosed herein. For example, if an antigen binding polypeptide disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3.

In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides or the antigen binding polypeptide complexes disclosed herein, comprise one or more CL, as indicated in the formulas representing the antigen binding polypeptides or the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides or the antigen binding polypeptide complexes disclosed herein, comprise one or more CH1, as indicated in the formulas representing the antigen binding polypeptides or the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides or the antigen binding polypeptide complexes disclosed herein, comprise one or more hinge regions, wherein a hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074.

In some aspects, the antigen binding polypeptides or the antigen binding polypeptide complexes disclosed herein, comprise one or more Fc region. In some aspects, the Fc region is at the carboxy-terminus of the antigen binding polypeptides disclosed herein. In some aspects, the Fc region is at the carboxy-terminus of at least one of the antigen binding polypeptides comprised in the antigen binding polypeptide complexes disclosed herein. In some aspects, the Fc region comprises at least one knob-into-hole modification or Fc effector function knockout mutation. In some aspects, the Fc region comprises at least one knob-into-hole modification or Fc effector function knockout mutation. In some aspects, the Fc region comprises an immunoglobulin heavy chain constant region 2 (CH2) and/or an immunoglobulin heavy chain constant region 3 (CH3), as indicated in the formulas representing the antigen binding polypeptides or the antigen binding polypeptide complexes disclosed herein. In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise one or more of the following amino acid substitutions:
M428L and N434A (LA);
M428L and N434S (LS);
L234F and L235E;
L234F, L235E, and P331S (TM); and/or
M252Y, S254T, and T256E (YTE);
or equivalent thereof, based on the EU numbering scheme.

In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence at least 65%, at least 70%, at least 75%, at least 80%, at least 85%, at least 90%, at least 95%, at least 99%, or 100% identical to any one of SEQ ID NOs: 376-380.

In some aspects, the Fc region comprises at least one knob-into-hole modification or Fc effector function knockout mutation. In some aspects, the antigen binding polypeptide or the antigen binding polypeptide complex disclosed herein is an IgG1 or IgG4 antibody or antigen binding fragment thereof and the knob-into-hole modification comprises:
(i) knob substitutions of S354C and T366W;
(ii) hole substitutions Y349C, T366S, L368A and Y407V;
(iii) a combination thereof;
or equivalent thereof, based on the EU numbering scheme.

In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptide or the antigen binding polypeptide complex disclosed herein is an IgG1 or IgG4 antibody or antigen binding fragment thereof and the Fc effector function knockout mutation is L234A, L235A, P239A, or equivalent thereof, or a combination thereof, based on the EU numbering scheme.

In some aspects, the antigen binding polypeptide or the antigen binding polypeptide complex disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 503 and the second polypeptide is encoded by a nucleotide sequence at least 65%, at least 70%, at least 75%, at least 80%, at least 85%, at least 90%, at least 95%, at least 99%, or 100% identical to SEQ ID NO: 503.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 504 and the second polypeptide is encoded by a nucleotide sequence at least 65%, at least 70%, at least 75%, at least 80%, at least 85%, at least 90%, at least 95%, at least 99%, or 100% identical to SEQ ID NO: 504.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 505 and the second polypeptide is encoded by a nucleotide sequence at least 65%, at least 70%, at least 75%, at least 80%, at least 85%, at least 90%, at least 95%, at least 99%, or 100% identical to SEQ ID NO: 505.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 506 and the second polypeptide is encoded by a nucleotide sequence at least 65%, at least 70%, at least 75%, at least 80%, at least 85%, at least 90%, at least 95%, at least 99%, or 100% identical to SEQ ID NO: 507.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 508 and the second polypeptide is encoded by a nucleotide sequence at least 65%, at least 70%, at least 75%, at least 80%, at least 85%, at least 90%, at least 95%, at least 99%, or 100% identical to SEQ ID NO: 509.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 510 and the second polypeptide is encoded by a nucleotide sequence at least 65%, at least 70%, at least 75%, at least 80%, at least 85%, at least 90%, at least 95%, at least 99%, or 100% identical to SEQ ID NO: 510.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 511 and the second polypeptide is encoded by a nucleotide sequence at least 65%, at least 70%, at least 75%, at least 80%, at least 85%, at least 90%, at least 95%, at least 99%, or 100% identical to SEQ ID NO: 511.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 512 and the second polypeptide is encoded by a nucleotide sequence at least 65%, at least 70%, at least 75%, at least 80%, at least 85%, at least 90%, at least 95%, at least 99%, or 100% identical to SEQ ID NO: 512.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 513 and the second polypeptide is encoded by a nucleotide sequence at least 65%, at least 70%, at least 75%, at least 80%, at least 85%, at least 90%, at least 95%, at least 99%, or 100% identical to SEQ ID NO: 513.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 514 and the second polypeptide is encoded by a nucleotide sequence at least 65%, at least 70%, at least 75%, at least 80%, at least 85%, at least 90%, at least 95%, at least 99%, or 100% identical to SEQ ID NO: 515.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 516 and the second polypeptide is encoded by a nucleotide sequence at least 65%, at least 70%, at least 75%, at least 80%, at least 85%, at least 90%, at least 95%, at least 99%, or 100% identical to SEQ ID NO: 517.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 518 and the second polypeptide is encoded by a nucleotide sequence at least 65%, at least 70%, at least 75%, at least 80%, at least 85%, at least 90%, at least 95%, at least 99%, or 100% identical to SEQ ID NO: 519.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 520 and the second polypeptide is encoded by a nucleotide sequence at least 65%, at least 70%, at least 75%, at least 80%, at least 85%, at least 90%, at least 95%, at least 99%, or 100% identical to SEQ ID NO: 521.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 522 and the second polypeptide is encoded by a nucleotide sequence at least 65%, at least 70%, at least 75%, at least 80%, at least 85%, at least 90%, at least 95%, at least 99%, or 100% identical to SEQ ID NO: 522.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 523 and the second polypeptide is encoded by a nucleotide sequence at least 65%, at least 70%, at least 75%, at least 80%, at least 85%, at least 90%, at least 95%, at least 99%, or 100% identical to SEQ ID NO: 523.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 524 and the second polypeptide is encoded by a nucleotide sequence at least 65%, at least 70%, at least 75%, at least 80%, at least 85%, at least 90%, at least 95%, at least 99%, or 100% identical to SEQ ID NO: 524.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 525 and the second polypeptide is encoded by a nucleotide sequence at least 65%, at least 70%, at least 75%, at least 80%, at least 85%, at least 90%, at least 95%, at least 99%, or 100% identical to SEQ ID NO: 525.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 526 and the second polypeptide is encoded by a nucleotide sequence at least 65%, at least 70%, at least 75%, at least 80%, at least 85%, at least 90%, at least 95%, at least 99%, or 100% identical to SEQ ID NO: 526.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 527 and the second polypeptide is encoded by a nucleotide sequence at least 65%, at least 70%, at least 75%, at least 80%, at least 85%, at least 90%, at least 95%, at least 99%, or 100% identical to SEQ ID NO: 528.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 529 and the second polypeptide is encoded by a nucleotide sequence at least 65%, at least 70%, at least 75%, at least 80%, at least 85%, at least 90%, at least 95%, at least 99%, or 100% identical to SEQ ID NO: 530.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 531 and the second polypeptide is encoded by a nucleotide sequence at least 65%, at least 70%, at least 75%, at least 80%, at least 85%, at least 90%, at least 95%, at least 99%, or 100% identical to SEQ ID NO: 532.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 533 and the second polypeptide is encoded by a nucleotide sequence at least 65%, at least 70%, at least 75%, at least 80%, at least 85%, at least 90%, at least 95%, at least 99%, or 100% identical to SEQ ID NO: 534.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 535 and the second polypeptide is encoded by a nucleotide sequence at least 65%, at least 70%, at least 75%, at least 80%, at least 85%, at least 90%, at least 95%, at least 99%, or 100% identical to SEQ ID NO: 536.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 537 and the second polypeptide is encoded by a nucleotide sequence at least 65%, at least 70%, at least 75%, at least 80%, at least 85%, at least 90%, at least 95%, at least 99%, or 100% identical to SEQ ID NO: 538.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 539 and the second polypeptide is encoded by a nucleotide sequence at least 65%, at least 70%, at least 75%, at least 80%, at least 85%, at least 90%, at least 95%, at least 99%, or 100% identical to SEQ ID NO: 540.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 541 and the second polypeptide is encoded by a nucleotide sequence at least 65%, at least 70%, at least 75%, at least 80%, at least 85%, at least 90%, at least 95%, at least 99%, or 100% identical to SEQ ID NO: 542.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 543 and the second polypeptide is encoded by a nucleotide sequence at least 65%, at least 70%, at least 75%, at least 80%, at least 85%, at least 90%, at least 95%, at least 99%, or 100% identical to SEQ ID NO: 544.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 545 and the second polypeptide is encoded by a nucleotide sequence at least 65%, at least 70%, at least 75%, at least 80%, at least 85%, at least 90%, at least 95%, at least 99%, or 100% identical to SEQ ID NO: 546.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 547 and the second polypeptide is encoded by a nucleotide sequence at least 65%, at least 70%, at least 75%, at least 80%, at least 85%, at least 90%, at least 95%, at least 99%, or 100% identical to SEQ ID NO: 548.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 549 and the second polypeptide is encoded by a nucleotide sequence at least 65%, at least 70%, at least 75%, at least 80%, at least 85%, at least 90%, at least 95%, at least 99%, or 100% identical to SEQ ID NO: 550.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 551 and the second polypeptide is encoded by a nucleotide sequence at least 65%, at least 70%, at least 75%, at least 80%, at least 85%, at least 90%, at least 95%, at least 99%, or 100% identical to SEQ ID NO: 552.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 553 and the second polypeptide is encoded by a nucleotide sequence at least 65%, at least 70%, at least 75%, at least 80%, at least 85%, at least 90%, at least 95%, at least 99%, or 100% identical to SEQ ID NO: 554.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 555 and the second polypeptide is encoded by a nucleotide sequence at least 65%, at least 70%, at least 75%, at least 80%, at least 85%, at least 90%, at least 95%, at least 99%, or 100% identical to SEQ ID NO: 556.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 557 and the second polypeptide is encoded by a nucleotide sequence at least 65%, at least 70%, at least 75%, at least 80%, at least 85%, at least 90%, at least 95%, at least 99%, or 100% identical to SEQ ID NO: 558.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 559 and the second polypeptide is encoded by a nucleotide sequence at least 65%, at least 70%, at least 75%, at least 80%, at least 85%, at least 90%, at least 95%, at least 99%, or 100% identical to SEQ ID NO: 560.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 561 and the second polypeptide is encoded by a nucleotide sequence at least 65%, at least 70%, at least 75%, at least 80%, at least 85%, at least 90%, at least 95%, at least 99%, or 100% identical to SEQ ID NO: 562.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 563 and the second polypeptide is encoded by a nucleotide sequence at least 65%, at least 70%, at least 75%, at least 80%, at least 85%, at least 90%, at least 95%, at least 99%, or 100% identical to SEQ ID NO: 564.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 565 and the second polypeptide is encoded by a nucleotide sequence at least 65%, at least 70%, at least 75%, at least 80%, at least 85%, at least 90%, at least 95%, at least 99%, or 100% identical to SEQ ID NO: 566.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 567 and the second polypeptide is encoded by a nucleotide sequence at least 65%, at least 70%, at least 75%, at least 80%, at least 85%, at least 90%, at least 95%, at least 99%, or 100% identical to SEQ ID NO: 568.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 569 and the second polypeptide is encoded by a nucleotide sequence at least 65%, at least 70%, at least 75%, at least 80%, at least 85%, at least 90%, at least 95%, at least 99%, or 100% identical to SEQ ID NO: 570.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 571 and the second polypeptide is encoded by a nucleotide sequence at least 65%, at least 70%, at least 75%, at least 80%, at least 85%, at least 90%, at least 95%, at least 99%, or 100% identical to SEQ ID NO: 572.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 573 and the second polypeptide is encoded by a nucleotide sequence at least 65%, at least 70%, at least 75%, at least 80%, at least 85%, at least 90%, at least 95%, at least 99%, or 100% identical to SEQ ID NO: 574.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 575 and the second polypeptide is encoded by a nucleotide sequence at least 65%, at least 70%, at least 75%, at least 80%, at least 85%, at least 90%, at least 95%, at least 99%, or 100% identical to SEQ ID NO: 576.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 577 and the second polypeptide is encoded by a nucleotide sequence at least 65%, at least 70%, at least 75%, at least 80%, at least 85%, at least 90%, at least 95%, at least 99%, or 100% identical to SEQ ID NO: 578.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 579 and the second polypeptide is encoded by a nucleotide sequence at least 65%, at least 70%, at least 75%, at least 80%, at least 85%, at least 90%, at least 95%, at least 99%, or 100% identical to SEQ ID NO: 580.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 581 and the second polypeptide is encoded by a nucleotide sequence at least 65%, at least 70%, at least 75%, at least 80%, at least 85%, at least 90%, at least 95%, at least 99%, or 100% identical to SEQ ID NO: 582.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 583 and the second polypeptide is encoded by a nucleotide sequence at least 65%, at least 70%, at least 75%, at least 80%, at least 85%, at least 90%, at least 95%, at least 99%, or 100% identical to SEQ ID NO: 584.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 585 and the second polypeptide is encoded by a nucleotide sequence at least 65%, at least 70%, at least 75%, at least 80%, at least 85%, at least 90%, at least 95%, at least 99%, or 100% identical to SEQ ID NO: 586.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 587 and the second polypeptide is encoded by a nucleotide sequence at least 65%, at least 70%, at least 75%, at least 80%, at least 85%, at least 90%, at least 95%, at least 99%, or 100% identical to SEQ ID NO: 588.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 589 and the second polypeptide is encoded by a nucleotide sequence at least 65%, at least 70%, at least 75%, at least 80%, at least 85%, at least 90%, at least 95%, at least 99%, or 100% identical to SEQ ID NO: 590.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 591 and the second polypeptide is encoded by a nucleotide sequence at least 65%, at least 70%, at least 75%, at least 80%, at least 85%, at least 90%, at least 95%, at least 99%, or 100% identical to SEQ ID NO: 591.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 592 and the second polypeptide is encoded by a nucleotide sequence at least 65%, at least 70%, at least 75%, at least 80%, at least 85%, at least 90%, at least 95%, at least 99%, or 100% identical to SEQ ID NO: 592.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 593 and the second polypeptide is encoded by a nucleotide sequence at least 65%, at least 70%, at least 75%, at least 80%, at least 85%, at least 90%, at least 95%, at least 99%, or 100% identical to SEQ ID NO: 593.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 594 and the second polypeptide is encoded by a nucleotide sequence at least 65%, at least 70%, at least 75%, at least 80%, at least 85%, at least 90%, at least 95%, at least 99%, or 100% identical to SEQ ID NO: 595.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 596 and the second polypeptide is encoded by a nucleotide sequence at least 65%, at least 70%, at least 75%, at least 80%, at least 85%, at least 90%, at least 95%, at least 99%, or 100% identical to SEQ ID NO: 597.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 598 and the second polypeptide is encoded by a nucleotide sequence at least 65%, at least 70%, at least 75%, at least 80%, at least 85%, at least 90%, at least 95%, at least 99%, or 100% identical to SEQ ID NO: 599.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 600 and the second polypeptide is encoded by a nucleotide sequence at least 65%, at least 70%, at least 75%, at least 80%, at least 85%, at least 90%, at least 95%, at least 99%, or 100% identical to SEQ ID NO: 600.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 601 and the second polypeptide is encoded by a nucleotide sequence at least 65%, at least 70%, at least 75%, at least 80%, at least 85%, at least 90%, at least 95%, at least 99%, or 100% identical to SEQ ID NO: 601.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 602 and the second polypeptide is encoded by a nucleotide sequence at least 65%, at least 70%, at least 75%, at least 80%, at least 85%, at least 90%, at least 95%, at least 99%, or 100% identical to SEQ ID NO: 602.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 603 and the second polypeptide is encoded by a nucleotide sequence at least 65%, at least 70%, at least 75%, at least 80%, at least 85%, at least 90%, at least 95%, at least 99%, or 100% identical to SEQ ID NO: 603.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 604 and the second polypeptide is encoded by a nucleotide sequence at least 65%, at least 70%, at least 75%, at least 80%, at least 85%, at least 90%, at least 95%, at least 99%, or 100% identical to SEQ ID NO: 604.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 605 and the second polypeptide is encoded by a nucleotide sequence at least 65%, at least 70%, at least 75%, at least 80%, at least 85%, at least 90%, at least 95%, at least 99%, or 100% identical to SEQ ID NO: 605.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 606 and the second polypeptide is encoded by a nucleotide sequence at least 65%, at least 70%, at least 75%, at least 80%, at least 85%, at least 90%, at least 95%, at least 99%, or 100% identical to SEQ ID NO: 606.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 607 and the second polypeptide is encoded by a nucleotide sequence at least 65%, at least 70%, at least 75%, at least 80%, at least 85%, at least 90%, at least 95%, at least 99%, or 100% identical to SEQ ID NO: 608.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 609 and the second polypeptide is encoded by a nucleotide sequence at least 65%, at least 70%, at least 75%, at least 80%, at least 85%, at least 90%, at least 95%, at least 99%, or 100% identical to SEQ ID NO: 610.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 611 and the second polypeptide is encoded by a nucleotide sequence at least 65%, at least 70%, at least 75%, at least 80%, at least 85%, at least 90%, at least 95%, at least 99%, or 100% identical to SEQ ID NO: 612.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 613 and the second polypeptide is encoded by a nucleotide sequence at least 65%, at least 70%, at least 75%, at least 80%, at least 85%, at least 90%, at least 95%, at least 99%, or 100% identical to SEQ ID NO: 614.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 615 and the second polypeptide is encoded by a nucleotide sequence at least 65%, at least 70%, at least 75%, at least 80%, at least 85%, at least 90%, at least 95%, at least 99%, or 100% identical to SEQ ID NO: 616.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 617 and the second polypeptide is encoded by a nucleotide sequence at least 65%, at least 70%, at least 75%, at least 80%, at least 85%, at least 90%, at least 95%, at least 99%, or 100% identical to SEQ ID NO: 618.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 619 and the second polypeptide is encoded by a nucleotide sequence at least 65%, at least 70%, at least 75%, at least 80%, at least 85%, at least 90%, at least 95%, at least 99%, or 100% identical to SEQ ID NO: 620.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 621 and the second polypeptide is encoded by a nucleotide sequence at least 65%, at least 70%, at least 75%, at least 80%, at least 85%, at least 90%, at least 95%, at least 99%, or 100% identical to SEQ ID NO: 622.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 623 and the second polypeptide is encoded by a nucleotide sequence at least 65%, at least 70%, at least 75%, at least 80%, at least 85%, at least 90%, at least 95%, at least 99%, or 100% identical to SEQ ID NO: 624.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 718 and the second polypeptide is encoded by a nucleotide sequence at least 65%, at least 70%, at least 75%, at least 80%, at least 85%, at least 90%, at least 95%, at least 99%, or 100% identical to SEQ ID NO: 719.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 722 and the second polypeptide is encoded by a nucleotide sequence at least 65%, at least 70%, at least 75%, at least 80%, at least 85%, at least 90%, at least 95%, at least 99%, or 100% identical to SEQ ID NO: 723.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 726 and the second polypeptide is encoded by a nucleotide sequence at least 65%, at least 70%, at least 75%, at least 80%, at least 85%, at least 90%, at least 95%, at least 99%, or 100% identical to SEQ ID NO: 727.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 730 and the second polypeptide is encoded by a nucleotide sequence at least 65%, at least 70%, at least 75%, at least 80%, at least 85%, at least 90%, at least 95%, at least 99%, or 100% identical to SEQ ID NO: 731.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 734 and the second polypeptide is encoded by a nucleotide sequence at least 65%, at least 70%, at least 75%, at least 80%, at least 85%, at least 90%, at least 95%, at least 99%, or 100% identical to SEQ ID NO: 735.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 738 and the second polypeptide is encoded by a nucleotide sequence at least 65%, at least 70%, at least 75%, at least 80%, at least 85%, at least 90%, at least 95%, at least 99%, or 100% identical to SEQ ID NO: 739.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 742 and the second polypeptide is encoded by a nucleotide sequence at least 65%, at least 70%, at least 75%, at least 80%, at least 85%, at least 90%, at least 95%, at least 99%, or 100% identical to SEQ ID NO: 743.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 746 and the second polypeptide is encoded by a nucleotide sequence at least 65%, at least 70%, at least 75%, at least 80%, at least 85%, at least 90%, at least 95%, at least 99%, or 100% identical to SEQ ID NO: 747.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 750 and the second polypeptide is encoded by a nucleotide sequence at least 65%, at least 70%, at least 75%, at least 80%, at least 85%, at least 90%, at least 95%, at least 99%, or 100% identical to SEQ ID NO: 751.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 754 and the second polypeptide is encoded by a nucleotide sequence at least 65%, at least 70%, at least 75%, at least 80%, at least 85%, at least 90%, at least 95%, at least 99%, or 100% identical to SEQ ID NO: 755.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 762 and the second polypeptide is encoded by a nucleotide sequence at least 65%, at least 70%, at least 75%, at least 80%, at least 85%, at least 90%, at least 95%, at least 99%, or 100% identical to SEQ ID NO: 763.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 927 and the second polypeptide is encoded by a nucleotide sequence at least 65%, at least 70%, at least 75%, at least 80%, at least 85%, at least 90%, at least 95%, at least 99%, or 100% identical to SEQ ID NO: 928.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 929 and the second polypeptide is encoded by a nucleotide sequence at least 65%, at least 70%, at least 75%, at least 80%, at least 85%, at least 90%, at least 95%, at least 99%, or 100% identical to SEQ ID NO: 930.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 931 and the second polypeptide is encoded by a nucleotide sequence at least 65%, at least 70%, at least 75%, at least 80%, at least 85%, at least 90%, at least 95%, at least 99%, or 100% identical to SEQ ID NO: 932.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 933 and the second polypeptide is encoded by a nucleotide sequence at least 65%, at least 70%, at least 75%, at least 80%, at least 85%, at least 90%, at least 95%, at least 99%, or 100% identical to SEQ ID NO: 934.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 935 and the second polypeptide is encoded by a nucleotide sequence at least 65%, at least 70%, at least 75%, at least 80%, at least 85%, at least 90%, at least 95%, at least 99%, or 100% identical to SEQ ID NO: 936.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 937 and the second polypeptide is encoded by a nucleotide sequence at least 65%, at least 70%, at least 75%, at least 80%, at least 85%, at least 90%, at least 95%, at least 99%, or 100% identical to SEQ ID NO: 938.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 939 and the second polypeptide is encoded by a nucleotide sequence at least 65%, at least 70%, at least 75%, at least 80%, at least 85%, at least 90%, at least 95%, at least 99%, or 100% identical to SEQ ID NO: 940.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 941 and the second polypeptide is encoded by a nucleotide sequence at least 65%, at least 70%, at least 75%, at least 80%, at least 85%, at least 90%, at least 95%, at least 99%, or 100% identical to SEQ ID NO: 942.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 943 and the second polypeptide is encoded by a nucleotide sequence at least 65%, at least 70%, at least 75%, at least 80%, at least 85%, at least 90%, at least 95%, at least 99%, or 100% identical to SEQ ID NO: 944.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 945 and the second polypeptide is encoded by a nucleotide sequence at least 65%, at least 70%, at least 75%, at least 80%, at least 85%, at least 90%, at least 95%, at least 99%, or 100% identical to SEQ ID NO: 946.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 947 and the second polypeptide is encoded by a nucleotide sequence at least 65%, at least 70%, at least 75%, at least 80%, at least 85%, at least 90%, at least 95%, at least 99%, or 100% identical to SEQ ID NO: 948.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 949 and the second polypeptide is encoded by a nucleotide sequence at least 65%, at least 70%, at least 75%, at least 80%, at least 85%, at least 90%, at least 95%, at least 99%, or 100% identical to SEQ ID NO: 950.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 951 and the second polypeptide is encoded by a nucleotide sequence at least 65%, at least 70%, at least 75%, at least 80%, at least 85%, at least 90%, at least 95%, at least 99%, or 100% identical to SEQ ID NO: 952.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 953 and the second polypeptide is encoded by a nucleotide sequence at least 65%, at least 70%, at least 75%, at least 80%, at least 85%, at least 90%, at least 95%, at least 99%, or 100% identical to SEQ ID NO: 954.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 955 and the second polypeptide is encoded by a nucleotide sequence at least 65%, at least 70%, at least 75%, at least 80%, at least 85%, at least 90%, at least 95%, at least 99%, or 100% identical to SEQ ID NO: 956.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 957 and the second polypeptide is encoded by a nucleotide sequence at least 65%, at least 70%, at least 75%, at least 80%, at least 85%, at least 90%, at least 95%, at least 99%, or 100% identical to SEQ ID NO: 958.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 959 and the second polypeptide is encoded by a nucleotide sequence at least 65%, at least 70%, at least 75%, at least 80%, at least 85%, at least 90%, at least 95%, at least 99%, or 100% identical to SEQ ID NO: 960.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 961 and the second polypeptide is encoded by a nucleotide sequence at least 65%, at least 70%, at least 75%, at least 80%, at least 85%, at least 90%, at least 95%, at least 99%, or 100% identical to SEQ ID NO: 962.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 963 and the second polypeptide is encoded by a nucleotide sequence at least 65%, at least 70%, at least 75%, at least 80%, at least 85%, at least 90%, at least 95%, at least 99%, or 100% identical to SEQ ID NO: 964.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 965 and the second polypeptide is encoded by a nucleotide sequence at least 65%, at least 70%, at least 75%, at least 80%, at least 85%, at least 90%, at least 95%, at least 99%, or 100% identical to SEQ ID NO: 966.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 978 and the second polypeptide is encoded by a nucleotide sequence at least 65%, at least 70%, at least 75%, at least 80%, at least 85%, at least 90%, at least 95%, at least 99%, or 100% identical to SEQ ID NO: 978.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 979 and the second polypeptide is encoded by a nucleotide sequence at least 65%, at least 70%, at least 75%, at least 80%, at least 85%, at least 90%, at least 95%, at least 99%, or 100% identical to SEQ ID NO: 979.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 980 and the second polypeptide is encoded by a nucleotide sequence at least 65%, at least 70%, at least 75%, at least 80%, at least 85%, at least 90%, at least 95%, at least 99%, or 100% identical to SEQ ID NO: 980.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 981 and the second polypeptide is encoded by a nucleotide sequence at least 65%, at least 70%, at least 75%, at least 80%, at least 85%, at least 90%, at least 95%, at least 99%, or 100% identical to SEQ ID NO: 981.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 982 and the second polypeptide is encoded by a nucleotide sequence at least 65%, at least 70%, at least 75%, at least 80%, at least 85%, at least 90%, at least 95%, at least 99%, or 100% identical to SEQ ID NO: 982.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 983 and the second polypeptide is encoded by a nucleotide sequence at least 65%, at least 70%, at least 75%, at least 80%, at least 85%, at least 90%, at least 95%, at least 99%, or 100% identical to SEQ ID NO: 983.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 986 and the second polypeptide is encoded by a nucleotide sequence at least 65%, at least 70%, at least 75%, at least 80%, at least 85%, at least 90%, at least 95%, at least 99%, or 100% identical to SEQ ID NO: 987.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 625 and the second polypeptide is encoded by a nucleotide sequence at least 65%, at least 70%, at least 75%, at least 80%, at least 85%, at least 90%, at least 95%, at least 99%, or 100% identical to SEQ ID NO: 625.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 991 and the second polypeptide is encoded by a nucleotide sequence at least 65%, at least 70%, at least 75%, at least 80%, at least 85%, at least 90%, at least 95%, at least 99%, or 100% identical to SEQ ID NO: 992.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 995 and the second polypeptide is encoded by a nucleotide sequence at least 65%, at least 70%, at least 75%, at least 80%, at least 85%, at least 90%, at least 95%, at least 99%, or 100% identical to SEQ ID NO: 996.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 999 and the second polypeptide is encoded by a nucleotide sequence at least 65%, at least 70%, at least 75%, at least 80%, at least 85%, at least 90%, at least 95%, at least 99%, or 100% identical to SEQ ID NO: 1000.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1003 and the second polypeptide is encoded by a nucleotide sequence at least 65%, at least 70%, at least 75%, at least 80%, at least 85%, at least 90%, at least 95%, at least 99%, or 100% identical to SEQ ID NO: 1004.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1007 and the second polypeptide is encoded by a nucleotide sequence at least 65%, at least 70%, at least 75%, at least 80%, at least 85%, at least 90%, at least 95%, at least 99%, or 100% identical to SEQ ID NO: 1008.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1011 and the second polypeptide is encoded by a nucleotide sequence at least 65%, at least 70%, at least 75%, at least 80%, at least 85%, at least 90%, at least 95%, at least 99%, or 100% identical to SEQ ID NO: 1012.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1015 and the second polypeptide is encoded by a nucleotide sequence at least 65%, at least 70%, at least 75%, at least 80%, at least 85%, at least 90%, at least 95%, at least 99%, or 100% identical to SEQ ID NO: 1016.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1019 and the second polypeptide is encoded by a nucleotide sequence at least 65%, at least 70%, at least 75%, at least 80%, at least 85%, at least 90%, at least 95%, at least 99%, or 100% identical to SEQ ID NO: 1020.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1023 and the second polypeptide is encoded by a nucleotide sequence at least 65%, at least 70%, at least 75%, at least 80%, at least 85%, at least 90%, at least 95%, at least 99%, or 100% identical to SEQ ID NO: 1024.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1027 and the second polypeptide is encoded by a nucleotide sequence at least 65%, at least 70%, at least 75%, at least 80%, at least 85%, at least 90%, at least 95%, at least 99%, or 100% identical to SEQ ID NO: 1028.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1031 and the second polypeptide is encoded by a nucleotide sequence at least 65%, at least 70%, at least 75%, at least 80%, at least 85%, at least 90%, at least 95%, at least 99%, or 100% identical to SEQ ID NO: 1032.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1035 and the second polypeptide is encoded by a nucleotide sequence at least 65%, at least 70%, at least 75%, at least 80%, at least 85%, at least 90%, at least 95%, at least 99%, or 100% identical to SEQ ID NO: 1036.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1039 and the second polypeptide is encoded by a nucleotide sequence at least 65%, at least 70%, at least 75%, at least 80%, at least 85%, at least 90%, at least 95%, at least 99%, or 100% identical to SEQ ID NO: 1040.

In some aspects, the first polypeptide is encoded by a nucleotide sequence at least 65%, at least 70%, at least 75%, at least 80%, at least 85%, at least 90%, at least 95%, at least 99%, or 100% identical to SEQ ID NO: 1043 and the second polypeptide is encoded by a nucleotide sequence at least 65%, at least 70%, at least 75%, at least 80%, at least 85%, at least 90%, at least 95%, at least 99%, or 100% identical to SEQ ID NO: 1044.

In some aspects, an antigen binding polypeptide or antigen binding polypeptide complex disclosed herein comprises a VL comprising a CDR1, a CDR2, and a CDR3 of any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, an antigen binding polypeptide or antigen binding polypeptide complex disclosed herein comprises a VH comprising a CDR1, a CDR2, and a CDR3 of any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, an antigen binding polypeptide or antigen binding polypeptide complex disclosed herein comprises a VL comprising a CDR1, a CDR2, and a CDR3 of any one of Bebtelovimab, Nirsevimab, Cilgavimab, Tixagevimab, Casirivimab, Imdevimab, Tocilizumab, AZD3152, MEDI8852, Bamlanivimab, Etesevimab, Sotrovimab, Regdanvimab, TY027, BRII-196+BRII-198, BI 767551 (DZIF-10c), SCTA01, ADG20, MAD0004J08, MW33, DXP593, COVI-AMG (STI-2020), LY-CoV1404, LY-CoV555, LY-CoV016, XVR011, 47D11, ADM03820, DXP604, C144-LS, C-135-LS, BGB-DXP593, AZD7442, and AZD5156.

In some aspects, an antigen binding polypeptide or antigen binding polypeptide complex disclosed herein comprises a VH comprising a CDR1, a CDR2, and a CDR3 of any one of Bebtelovimab, Nirsevimab, Cilgavimab, Tixagevimab, Casirivimab, Imdevimab, Tocilizumab, AZD3152, MEDI8852, Bamlanivimab, Etesevimab, Sotrovimab, Regdanvimab, TY027, BRII-196+BRII-198, BI 767551 (DZIF-10c), SCTA01, ADG20, MAD0004J08, MW33, DXP593, COVI-AMG (STI-2020), LY-CoV1404, LY-CoV555, LY-CoV016, XVR011, 47D11, ADM03820, DXP604, C144-LS, C-135-LS, BGB-DXP593, AZD7442, and AZD5156.

In some aspects, an antigen binding polypeptide or antigen binding polypeptide complex disclosed herein comprise a detectable label. In some aspects, the detectable label is a radioactive label, chemiluminescent label, fluorescent label, enzyme, peptide tag, or a combination thereof. In some aspects, the peptide tag is a poly histidine tag consisting of from about four to about 10 histidine residues. In some aspects, the poly histidine tag consists of about eight histidine residues.

In some aspects, an antigen binding polypeptide or antigen binding polypeptide complex disclosed herein is conjugated to an agent as an antibody-drug conjugate (ADC). In some aspects, the agent is a cytotoxic agent, immunomodulating agent, imaging agent, or therapeutic protein, or a combination thereof.

In some aspects, an antigen binding polypeptide or antigen binding polypeptide complex disclosed herein specifically binds to the SARS-CoV-2 protein with an equilibrium dissociation constant (K_{D}) of from about 10 μM to about 1 pM.

In some aspects, an antigen binding polypeptide, antigen binding polypeptide complex, or antibody or antigen binding fragment thereof disclosed herein is or comprises a class I antibody or antigen binding fragment thereof; class II antibody or antigen binding fragment thereof; class III antibody or antigen binding fragment thereof; class IV antibody or antigen binding fragment thereof; class I and II antibody or antigen binding fragment thereof; class I and III antibody or antigen binding fragment thereof; class I and IV antibody or antigen binding fragment thereof; class II and III antibody or antigen binding fragment thereof; class II and IV antibody or antigen binding fragment thereof; class III and IV antibody or antigen binding fragment thereof; class I, II and III antibody or antigen binding fragment thereof; class I, II and IV antibody or antigen binding fragment thereof; class I, III and IV antibody or antigen binding fragment thereof; class II, III and IV antibody or antigen binding fragment thereof; or class I, II, III and IV antibody or antigen binding fragment thereof. According to the published literature, anti-SARS-CoV-2 antibodies are generally divided into four main classes depending on the epitopes they target in the RBD (receptor binding domain) of the S (spike) protein. Several different classification systems have been proposed, and the proposed classification systems are not rigid, with antibodies displaying characteristics and properties of more than one antibody classes. An exemplary classification, and the most commonly referenced, is that proposed by Barnes et al. (Barnes C O, et al., SARS-CoV-2 neutralizing antibody structures inform therapeutic strategies. Nature. 2020 December; 588(7839):682-687). Class 1 antibodies (or antigen binding fragments thereof) compete with ACE2 for binding site and only recognize “up” RBD. Class 2 antibodies (or antigen binding fragments thereof) are capable of binding to RBD in both “up” and “down” conformations. As with Class 1, they also compete with ACE2 for RBD binding. Class 3 antibodies (or antigen binding fragments thereof) bind to the RBD on the opposite side of Class 1 and Class 2 binding epitope close to an N-glycan attached to residue N343. Class 4 antibodies (or antigen binding fragments thereof) target a cryptic epitope that faces the interior of the S protein on “up” RBDs. Some anti-SARS-CoV-2 neutralizing antibodies (or antigen binding fragments thereof) can display properties of more than one Classes of anti-SARS-CoV-2 antibodies. For example, an antibody (e.g., A18-448, or antigen binding fragments thereof) can bind to an epitope to which antibodies (or antigen binding fragments thereof) belonging to one of the four anti-SARS-CoV-2 antibody Classes bind and can compete for binding with one or more antibodies (or antigen binding fragments thereof) belonging to one or more different Classes of anti-SARS-CoV-2 antibodies. For example, A18-448 (or antigen binding fragments thereof) binds to an epitope located in a region of RBD to which Class IV anti-SARS-CoV-2 antibodies (or antigen binding fragments thereof) bind, but can compete for binding to RBD with anti-SARS-CoV-2 antibodies (or antigen binding fragments thereof) belonging to Class I and Class III. Thus, A18-448 can be described, for example, as a Class I/IV anti-SARS-CoV-2 antibody (or antigen binding fragment thereof).

According to the published literature, anti-SARS-CoV-2 antibodies can also be divided into a different number of classes, for example, into five main classes (see, for example, Starr. T. N., Czudnochowski, N., Liu. Z, et al, SARS-CoV-2 RBD antibodies that maximize breadth and resistance to escape. Nature 597, 97-102 (2021). Thus, in some aspects, an antigen binding polypeptide, antigen binding polypeptide complex, or antibody or antigen binding fragment thereof disclosed herein can be or can comprise a class V antibody (or antigen binding fragments thereof), or an antibody (or antigen binding fragments thereof) having a combination of classes including class V and one or more other classes described herein (e.g., class I, class II, class III, and/or class IV).

In some aspects, an antigen binding polypeptide complex disclosed herein is bispecific, trispecific or tetraspecific and has greater neutralization potency against SARS-CoV-2 virus than a monospecific antigen binding polypeptide complex that specifically binds to one of the same antigens as the bispecific, trispecific or tetraspecific antigen binding polypeptide complex.

In some aspects, an antigen binding polypeptide complex disclosed herein is bispecific, trispecific or tetraspecific and has greater neutralization potency against SARS-CoV-2 virus than a mixture of monospecific antigen binding polypeptide complexes that specifically bind to the same antigens as the bispecific, trispecific or tetraspecific antigen binding polypeptide complex.

In some aspects, the antigen binding polypeptides or the antigen binding polypeptide complexes disclosed herein bind to a SARS-CoV-2 virus. In some aspects, the SARS-CoV-2 virus is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an lota variant (e.g., B.1.526), a Lambda variant (e.g., C.37), an Epsilon variant (e.g., B.1.429, B.1.427 or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, or XBB.2.3.2), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof. In some aspects, the Omicron variant is B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, KP.3.1.1, or a combination or variant thereof.

In some aspects, the antigen binding polypeptides and polypeptide complexes disclosed herein have broad reactivity against a viral antigen selected from the group consisting of influenza virus neuraminidase, influenza virus hemagglutinin, human respiratory syncytial virus (RSV)-viral proteins, RSV F glycoprotein, RSV G glycoprotein, herpes simplex virus (HSV) viral proteins, herpes simplex virus glycoproteins gB, gC, gD, and gE, chlamydia MOMP and PorB antigens, core protein, matrix protein or other protein of Dengue virus, measles virus hemagglutinin, herpes simplex virus type 2 glycoprotein gB, poliovirus 1 VP1, envelope glycoproteins of HIV 1, hepatitis B surface antigen, diptheria toxin, streptococcus 24M epitope, gonococcal pilin, pseudorabies virus g50) (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55. Newcastle disease virus hemagglutinin-neuraminidase, swine flu hemagglutinin, swine flu neuraminidase, foot and mouth disease virus, hog colera virus, swine influenza virus. African swine fever virus. Mycoplasma liyopneutiioniae, infectious bovine rhinotracheitis virus, infectious bovine rhinotracheitis virus glycoprotein E, glycoprotein G, infectious laryngotracheitis virus, infectious laryngotracheitis virus glycoprotein G or glycoprotein I, a glycoprotein of La Crosse virus, neonatal calf diarrhoea virus. Venezuelan equine encephalomyelitis virus, punta toro virus, murine leukemia virus, mouse mammary tumor virus, hepatitis B virus core protein and hepatitis B virus surface antigen or a fragment or derivative thereof, antigen of equine influenza virus or equine herpes virus, including equine influenza virus type A/Alaska 91 neuraminidase, equine influenza virus typeA/Miami 63 neuraminidase, equine influenza virus type A/Kentucky 81 neuraminidase equine herpes virus type 1 glycoprotein B, and equine herpes virus type 1 glycoprotein D, antigen of bovine respiratory syncytial virus or bovine parainfluenza virus, bovine respiratory syncytial virus attachment protein (BRSV G), bovine respiratory syncytial virus fusion protein (BRSV F), bovine respiratory syncytial virus nucleocapsid protein (BRSVN), bovine parainfluenza virus type 3 fusion protein, bovine parainfluenza virus type 3 hemagglutinin neuraminidase, bovine E viral diarrhoea virus glycoprotein 48 and glycoprotein 53, glycoprotein E of Dengue virus, and glycoprotein E1E2 of human hepatitis C virus.

In some aspects, the antigen binding polypeptides and polypeptide complexes disclosed herein bind to at least one epitope on at least one antigen selected from the group consisting of influenza virus neuraminidase, influenza virus hemagglutinin, human respiratory syncytial virus (RSV)-viral proteins, RSV F glycoprotein, RSV G glycoprotein, herpes simplex virus (HSV) viral proteins, herpes simplex virus glycoproteins gB, gC, gD, and gE, chlamydia MOMP and PorB antigens, core protein, matrix protein or other protein of Dengue virus, measles virus hemagglutinin, herpes simplex virus type 2 glycoprotein gB, poliovirus 1 VP1, envelope glycoproteins of HIV 1, hepatitis B surface antigen, diptheria toxin, streptococcus 24M epitope, gonococcal pilin, pseudorabies virus g50) (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein. Serpulina hydodysenteriae protective antigen, bovine viral diarrhea glycoprotein 55. Newcastle disease virus hemagglutinin-neuraminidase, swine flu hemagglutinin, swine flu neuraminidase, foot and mouth disease virus, hog colera virus, swine influenza virus. African swine fever virus, Mycoplasma liyopneutiioniae, infectious bovine rhinotracheitis virus, infectious bovine rhinotracheitis virus glycoprotein E, glycoprotein G, infectious laryngotracheitis virus, infectious laryngotracheitis virus glycoprotein G or glycoprotein I, a glycoprotein of La Crosse virus, neonatal calf diarrhoea virus. Venezuelan equine encephalomyelitis virus, punta toro virus, murine leukemia virus, mouse mammary tumor virus, hepatitis B virus core protein and hepatitis B virus surface antigen or a fragment or derivative thereof, antigen of equine influenza virus or equine herpes virus, including equine influenza virus type A/Alaska 91 neuraminidase, equine influenza virus typeA/Miami 63 neuraminidase, equine influenza virus type A/Kentucky 81 neuraminidase equine herpes virus type 1 glycoprotein B, and equine herpes virus type 1 glycoprotein D, antigen of bovine respiratory syncytial virus or bovine parainfluenza virus, bovine respiratory syncytial virus attachment protein (BRSV G), bovine respiratory syncytial virus fusion protein (BRSV F), bovine respiratory syncytial virus nucleocapsid protein (BRSVN), bovine parainfluenza virus type 3 fusion protein, bovine parainfluenza virus type 3 hemagglutinin neuraminidase, bovine E viral diarrhoea virus glycoprotein 48 and glycoprotein 53, glycoprotein E of Dengue virus, and glycoprotein E1E2 of human hepatitis C virus.

In some aspects, the antigen binding polypeptides and polypeptide complexes disclosed herein specifically bind to at least one epitope on at least one antigen selected from the group consisting of A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25 CCR3, CCR4, CD3, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL1O, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, and WUCAM.

In some aspects, the antigen binding polypeptides or the antigen binding polypeptide complexes disclosed herein comprise a VH comprising a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S.

In some aspects, the antigen binding polypeptides or the antigen binding polypeptide complexes disclosed herein have an isoelectric point (pI) of from about 7 to about 9.

In some aspects, provided herein are antibodies or antigen binding fragments thereof comprising the antigen binding polypeptides or antigen binding polypeptide complexes disclosed herein. In some aspects, an antibody or antigen binding fragment thereof comprising the antigen binding polypeptides or antigen binding polypeptide complexes disclosed herein is IgG, IgM, IgE, IgA or IgD. In some aspects, the IgG is IgG1, IgG2, IgG3 or IgG4. In some aspects, an antibody or antigen binding fragment thereof comprising the antigen binding polypeptides or antigen binding polypeptide complexes disclosed herein is a Fab, scFab, Fab′, F(ab′)₂, Fv or scFv. In some aspects, an antibody or antigen binding fragment thereof comprising the antigen binding polypeptides or antigen binding polypeptide complexes disclosed herein is human or humanized.

In some aspects, provided herein are polynucleotides encoding the antigen binding polypeptides or antigen binding polypeptide complexes disclosed herein, or the antibodies or antigen binding fragments thereof comprising the antigen binding polypeptides or antigen binding polypeptide complexes disclosed herein. In some aspects, the polynucleotides disclosed herein comprise a nucleotide sequence having at least 65%, at least 70%, at least 75%, at least 80%, at least 85%, at least 90%, at least 95%, at least 99%, or 100% identity to a nucleotide sequence of any one of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044.

In some aspects, provided herein are vectors comprising the polynucleotides disclosed herein. In some aspects, provided herein are host cells comprising the vectors disclosed herein.

In some aspects, provided herein are mRNAs encoding the antigen binding polypeptides, antigen binding polypeptide complexes, or the antibodies or antigen binding fragments thereof provided herein. In some aspects, the mRNAs provided herein comprise a 5′-cap and/or a poly(A) tail.

In some aspects, provided herein are lipid nanoparticles (LNP) comprising the mRNAs provided herein.

In some aspects, provided herein are chimeric antigen receptor (CAR) comprising the antigen binding polypeptide, antigen binding polypeptide complexes, or antibodies or antigen binding fragments thereof disclosed herein. In some aspects, provided herein are immune cells comprising the CARs disclosed herein.

In some aspects, provided herein are pharmaceutical compositions comprising the antigen binding polypeptides, the antigen binding polypeptide complexes, the antibodies or antigen binding fragments thereof, the polynucleotides, the vectors, the host cells, the mRNAs, the LNPs, the CARs, the immune cells disclosed herein, or any combination thereof. In some aspects, the pharmaceutical compositions disclosed herein further comprise a pharmaceutically acceptable carrier, an additional pharmaceutical agent, or a combination thereof.

In some aspects, provided herein are kits comprising the antigen binding polypeptides, the antigen binding polypeptide complexes, the antibodies or antigen binding fragments thereof, the polynucleotides, the vectors, the host cells, the mRNAs, the LNPs, the CARs, the immune cells, the pharmaceutical compositions disclosed herein, or any combination thereof.

In some aspects, provided herein are methods of preventing or treating a SARS-CoV-2 viral infection in a subject in need thereof, comprising administering to the subject a therapeutically effective amount of the antigen binding polypeptides, the antigen binding polypeptide complexes, the antibodies or antigen binding fragments thereof, the polynucleotides, the vectors, the host cells, the mRNAs, the LNPs, the CARs, the immune cells, the pharmaceutical compositions disclosed herein, or any combination thereof.

In some aspects, provided herein are methods of preventing or treating coronavirus disease 2019 (COVID-19) in a subject in need thereof, comprising administering to the subject a therapeutically effective amount of the antigen binding polypeptides, the antigen binding polypeptide complexes, the antibodies or antigen binding fragments thereof, the polynucleotides, the vectors, the host cells, the mRNAs, the LNPs, the CARs, the immune cells, the pharmaceutical compositions disclosed herein, or any combination thereof.

In some aspects, provided herein are methods of diagnosing a subject as being infected with a SARS-CoV-2 virus or suspected of being infected with a SARS-CoV-2 virus, comprising (i) contacting a sample obtained from the subject with the antigen binding polypeptides, the antigen binding polypeptide complexes, the antibodies or antigen binding fragments thereof disclosed herein, or any combination thereof; (ii) detecting the presence or absence of a virus complex which contains the polypeptide or a fragment thereof, polypeptide complex or a fragment thereof, antibody or a fragment thereof and a SARS-CoV-2 virus, virion or fragment thereof; and (iii) diagnosing the subject as being infected with a SARS-CoV-2 virus or suspected of being infected with a SARS-CoV-2 virus when the presence of the virus complex is detected.

In some aspects, provided herein are methods of diagnosing a subject as not being infected with a SARS-CoV-2 virus or not suspected of being infected with a SARS-CoV-2 virus, comprising (i) contacting a sample obtained from the subject with the antigen binding polypeptides, the antigen binding polypeptide complexes, the antibodies or antigen binding fragments thereof disclosed herein, or any combination thereof; (ii) detecting the presence or absence of a virus complex which contains the polypeptide or a fragment thereof, polypeptide complex or a fragment thereof, antibody or a fragment thereof and a SARS-CoV-2 virus, virion or fragment thereof; and (iii) diagnosing the subject as not being infected with a SARS-CoV-2 virus or not suspected of being infected with a SARS-CoV-2 virus when the presence of the virus complex is not detected.

In some aspects, provided herein are methods of diagnosing a subject as having COVID-19 or suspected of having COVID-19, comprising (i) contacting a sample obtained from the subject with the antigen binding polypeptides, the antigen binding polypeptide complexes, the antibodies or antigen binding fragments thereof disclosed herein, or any combination thereof; (ii) detecting the presence or absence of a virus complex which contains the polypeptide or a fragment thereof, polypeptide complex or a fragment thereof, antibody or a fragment thereof and a SARS-CoV-2 virus, virion or fragment thereof; and (iii) diagnosing the subject as having COVID-19 or suspected of having COVID-19 when the presence of the virus complex is detected.

In some aspects, provided herein are methods of diagnosing a subject as not being infected with a SARS-CoV-2 virus or not suspected of being infected with a SARS-CoV-2 virus, comprising (i) contacting a sample obtained from the subject with the antigen binding polypeptides, the antigen binding polypeptide complexes, the antibodies or antigen binding fragments thereof disclosed herein, or any combination thereof; (ii) detecting the presence or absence of a virus complex which contains the polypeptide or a fragment thereof, polypeptide complex or a fragment thereof, antibody or a fragment thereof and a SARS-CoV-2 virus, virion or fragment thereof; and (iii) diagnosing the subject as not being infected with a SARS-CoV-2 virus or not suspected of being infected with a SARS-CoV-2 virus when the presence of the virus complex is not detected.

In some aspects, provided herein are methods of diagnosing a subject as having COVID-19 or suspected of having COVID-19, comprising (i) contacting a sample obtained from the subject with the antigen binding polypeptides, the antigen binding polypeptide complexes, the antibodies or antigen binding fragments thereof disclosed herein, or any combination thereof; (ii) detecting the presence or absence of a virus complex which contains the polypeptide or a fragment thereof, polypeptide complex or a fragment thereof, antibody or a fragment thereof and a SARS-CoV-2 virus, virion or fragment thereof; and (iii) diagnosing the subject as having COVID-19 or suspected of having COVID-19 when the presence of the virus complex is detected.

In some aspects, provided herein are of diagnosing a subject as not having COVID-19 or not suspected of having COVID-19, comprising (i) contacting a sample obtained from the subject with the antigen binding polypeptides, the antigen binding polypeptide complexes, the antibodies or antigen binding fragments thereof disclosed herein, or any combination thereof; (ii) detecting the presence or absence of a virus complex which contains the polypeptide or a fragment thereof, polypeptide complex or a fragment thereof, antibody or a fragment thereof and a SARS-CoV-2 virus, virion or fragment thereof; and (iii) diagnosing the subject as not having COVID-19 or not suspected of having COVID-19 when the presence of the virus complex is not detected.

In some aspects, the samples use in the methods provided herein are a nasal swab, tissue sample, saliva, plasma or blood. In some aspects, the methods provided herein comprise detecting the presence or absence of the virus complex comprises an enzyme linked immunosorbent assay (ELISA), immunospot assay, lateral flow assay, flow cytometry, immunohistochemistry or western blot.

In some aspects, provided herein are anti-SARS-CoV-2 virus antibodies or an antigen binding fragments thereof, wherein the anti-SARS-CoV-2 virus antibodies or an antigen binding fragments thereof comprise: (a) one or more immunoglobulin light chain constant domain (CL) and (b) one or more immunoglobulin heavy chain 1 constant domain (CH1); and wherein the anti-SARS-CoV-2 virus antibodies or an antigen binding fragments thereof (i) have an isoelectric point (pI), and after administered to a subject has (ii) a higher peak serum levels, and (iii) a reduced clearance levels, as compared to a same antibody or antigen binding fragments thereof not comprising one or more immunoglobulin light chain constant domain (CL) and (b) one or more immunoglobulin heavy chain 1 constant domain (CH1).

It is to be understood that “a same antibody not comprising a CL and a CH1” refers to an antibody (or antigen binding fragment thereof) having a same amino acid sequence as the reference antibody (or antigen binding fragment thereof) exception made for the CL and the CH1 being absent from the “same antibody not comprising a CL and a CH1.” For example, a reference antibody (or antigen binding fragment thereof) (antibody R, or antigen binding fragment thereof) may comprise an amino acid sequence represented by the formula VL1-CL-VL2-VH2-VH1-CH1. “a same antibody not comprising a CL and a CH1” (antibody R′, or antigen binding fragment thereof) it is to be understood as comprising an amino acid sequence that is represented by the formula VL1-VL2-VH2-VH1, wherein the VL1, the VL2, the VH1, and the VH1 in the antibody R (or antigen binding fragment thereof) comprise an amino acid sequence identical to the amino acid sequence comprised in the VL1, the VL2, the VH1, and the VH1 comprised in the antibody R′ (or antigen binding fragment thereof).

In some aspects, an anti-SARS-CoV-2 virus antibody or an antigen binding fragment thereof disclosed herein is an IgG, IgM, IgE, IgA or an IgD antibody or an antigen binding fragment thereof. In some aspects, an anti-SARS-CoV-2 virus antibody or an antigen binding fragment thereof disclosed herein is IgG1, IgG2, IgG3 or IgG4. In some aspects, an anti-SARS-CoV-2 virus antibody or an antigen binding fragment thereof disclosed herein is a Fab, scFab, Fab′, F(ab′)₂, Fv or scFv. In some aspects, an anti-SARS-CoV-2 virus antibody or an antigen binding fragment thereof disclosed herein is human or humanized. In some aspects, an anti-SARS-CoV-2 virus antibody or an antigen binding fragment thereof disclosed herein has an isoelectric point (pI) of from about 7 to about 9. In some aspects, an anti-SARS-CoV-2 virus antibody or an antigen binding fragment thereof disclosed herein binds to the SARS-CoV-2 protein with an equilibrium dissociation constant (K_{D}) of from about 10 μM to about 1 pM. In some aspects, an anti-SARS-CoV-2 virus antibody or an antigen binding fragment thereof disclosed herein is a class I antibody or antigen binding fragment thereof; class II antibody or antigen binding fragment thereof; class III antibody or antigen binding fragment thereof; class IV antibody or antigen binding fragment thereof; class I and II antibody or antigen binding fragment thereof; class I and III antibody or antigen binding fragment thereof; class I and IV antibody or antigen binding fragment thereof; class II and III antibody or antigen binding fragment thereof; class II and IV antibody or antigen binding fragment thereof; class III and IV antibody or antigen binding fragment thereof; class I, II and III antibody or antigen binding fragment thereof; class I, II and IV antibody or antigen binding fragment thereof; class I, III and IV antibody or antigen binding fragment thereof; class II, III and IV antibody or antigen binding fragment thereof; or class I, II, III and IV antibody or antigen binding fragment thereof. In some aspects, an anti-SARS-CoV-2 neutralizing antibody or antigen binding fragment thereof disclosed herein can display properties of more than one Classes of anti-SARS-CoV-2 antibodies or antigen binding fragments thereof. For example, an antibody (or antigen binding fragment thereof, e.g., A18-448 or antigen binding fragment thereof) can bind to an epitope to which antibodies (or antigen binding fragments thereof) belonging to one of the four anti-SARS-CoV-2 antibody Classes bind and can compete for binding with one or more antibodies (or antigen binding fragments thereof) belonging to one or more different Classes of anti-SARS-CoV-2 antibodies. For example, A18-448 (or antigen binding fragments thereof) binds to an epitope located in a region of RBD to which Class IV anti-SARS-CoV-2 antibodies (or antigen binding fragments thereof) bind, but can compete for binding to RBD with anti-SARS-CoV-2 antibodies (or antigen binding fragments thereof) belonging to Class I and Class III. Thus, A18-448 antibody or antigen binding fragments thereof can be described, for example, as a Class I/IV anti-SARS-CoV-2 antibody or antigen binding fragments thereof.

In some aspects, an anti-SARS-CoV-2 virus antibody or an antigen binding fragment thereof disclosed herein is bispecific, trispecific or tetraspecific and has greater neutralization potency against SARS-CoV-2 virus than a monospecific anti-SARS-CoV-2 virus antibody or an antigen binding fragment thereof that specifically binds to one of the same antigens as the bispecific, trispecific or tetraspecific anti-SARS-CoV-2 virus antibody or an antigen binding fragment thereof. In some aspects, an anti-SARS-CoV-2 virus antibody or an antigen binding fragment thereof disclosed herein is bispecific, trispecific or tetraspecific and has greater neutralization potency against SARS-CoV-2 virus than a mixture of monospecific anti-SARS-CoV-2 virus antibodies or an antigen binding fragments thereof that specifically binds to one of the same antigens as the bispecific, trispecific or tetraspecific anti-SARS-CoV-2 virus antibody or an antigen binding fragment thereof.

In some aspects, an anti-SARS-CoV-2 virus antibody or an antigen binding fragment thereof disclosed herein binds to a SARS-CoV-2 virus, and the SARS-CoV-2 virus is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an lota variant (e.g., B.1.526), a Lambda variant (e.g., C.37), an Epsilon variant (e.g., B.1.429, B.1.427, or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, or XBB.2.3.2), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof. In some aspects, Omicron variant is B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, KP.3.1.1, or a combination or variant thereof.

In some aspects, an anti-SARS-CoV-2 virus antibody or an antigen binding fragment thereof disclosed herein comprises one or more variable heavy chain regions (VH) and at least one of the one or more VH has a mutation at an N-glycosylation site. In some aspects, the mutation is at amino acid N62 of the VH region or equivalent thereof. In some aspects, the N62 mutation is N62Q or N62S, or equivalent thereof.

In some aspects, an anti-SARS-CoV-2 virus antibody or an antigen binding fragment thereof disclosed herein comprises at least one variable heavy chain region (VH) and/or at least one variable light chain region (VL).

In some aspects, an anti-SARS-CoV-2 virus antibody or an antigen binding fragment thereof disclosed herein comprises at least one variable light chain region (VL). In some aspects, the at least one VL comprises: (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, an anti-SARS-CoV-2 virus antibody or an antigen binding fragment thereof disclosed herein comprises at least one variable heavy chain region (VH). In some aspects, the at least one VH comprises: (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, an anti-SARS-CoV-2 virus antibody or an antigen binding fragment thereof disclosed herein comprises at least one variable light chain region (VL). In some aspects, the at least one VL comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, an anti-SARS-CoV-2 virus antibody or an antigen binding fragment thereof disclosed herein comprises at least one variable heavy chain region (VH). In some aspects, the at least one VH comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, an anti-SARS-CoV-2 virus antibody or an antigen binding fragment thereof disclosed herein is encoded by a nucleotide sequence at least 65%, at least 70%, at least 75%, at least 80%, at least 85%, at least 90%, at least 95%, at least 99%, or 100% identical to any one of SEQ ID NO: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044.

In some aspects, an anti-SARS-CoV-2 virus antibody or an antigen binding fragment thereof disclosed herein comprises an amino acid sequence at least 65%, at least 70%, at least 75%, at least 80%, at least 85%, at least 90%, at least 95%, at least 99%, or 100% identical to any one of SEQ ID NO: 381-502, 632-633, 720-721, 724-725, 728-729, 732-733, 736-737, 740-741, 744-745, 748-749, 752-753, 760-761, 887-926, 972-977, 984-985, 989-990, 993-994, 997-998, 1001-1002, 1005-1006, 1009-1010, 1013-1014, 1017-1018, 1021-1022, 1025-1026, 1029-1030, 1033-1034, 1037-1038, and 1041-1042.

Molecular biology and recombinant DNA methods for making, screening and engineering an antigen binding polypeptide or antigen binding polypeptide complex (e.g., an antibody or antigen binding fragment thereof) containing such sequences are well known and described, for example, in Adair et al. Human Antibodies, 5(1-2):41-47, 1994; Kostelny et al., J Immunol, 148(5):1547-1553, 1992. Shiraiwa et al., Methods, 154:10-20, 2019; and Zola. “Monoclonal Antibodies: A Manual of Techniques.” 1987, 1st Ed., CRC Press; and Steinitz. Human Antibodies, 18(1-2):1-10, 2009.

As used herein, an antigen binding polypeptide or antigen binding polypeptide complex (e.g., an antibody or antigen binding fragment thereof), or region or domain thereof that “specifically binds” refers to its association with an epitope by its antigen binding domain, and that the binding entails some complementarity between the antigen binding domain and the epitope. Specific binding to an epitope occurs where there is binding to that epitope via its antigen binding domain more readily than there would be binding to a random, unrelated epitope.

As used herein, an “epitope” refers to a localized region of an antigen to which an antigen binding polypeptide or antigen binding polypeptide complex (e.g., antibody or antigen binding fragment thereof) can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy. X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). See, e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303; Meth Enzymol (1985) volumes 114 & 115, eds Wyckoff H W et al., U.S. Pub. No. 2004/0014194). Bricogne G (1993) Acta Crystallogr D Biol Crystallogr 49 (Pt 1): 37-60, Bricogne G (1997) Meth Enzymol 276A: 361-423, ed Carter C W, and Roversi et al., (2000) Acta Crystallogr D Biol Crystallogr 56 (Pt 10): 1316-1323 (X-ray diffraction crystallography studies); and Champe et al., (1995) J Biol Chem 270:1388-1394 and Cunningham B C & Wells J A (1989) Science 244:1081-1085 (mutagenesis mapping).

Specific binding can be represented by a “binding affinity.” Binding affinity refers to an intrinsic binding affinity which reflects a 1:1 interaction between members of a binding pair (e.g., an antigen binding polypeptide complex and an antigen). Binding affinity can be measured and/or expressed in several ways known in the art, including, but not limited to, equilibrium dissociation constant (K_{D}), K_{D} is calculated from the quotient of k_{off}/kₒₙ, where kₒₙ refers to the association rate constant of, e.g., an antigen binding polypeptide complex to an antigen, and k_{off} refers to the dissociation of, e.g., an antigen binding polypeptide complex from an antigen. The kₒₙ and k_{off} can be determined by techniques known to one of ordinary skill in the art, such as Octet® BLI, BIAcore® or KinExA.

Accordingly, in some aspects, an antigen binding polypeptide complex provided herein is an antibody or antigen binding fragment thereof. In some aspects, an antigen binding polypeptide provided herein is part of an antibody or antigen binding fragment thereof.

In one aspect, the antibody or antigen binding fragment thereof specifically binds to an antigen with an equilibrium dissociation constant (K_{D})) of from about 10 μM to about 1 pM. In another aspect, the antibody or antigen binding fragment thereof is IgG, IgM, IgE, IgA or IgD. In another aspect, the IgG is IgG1, IgG2, IgG3 or IgG4. In another aspect, the antigen binding fragment is a Fab, scFab, Fab′, F(ab′)₂, Fv or scFv. In yet another aspect, the antibody or antigen binding fragment thereof is human or humanized.

In another aspect, an antigen binding polypeptide or an antigen binding polypeptide complex provided herein (e.g., an antibody or antigen binding fragment thereof) is trivalent or tetravalent.

In another aspect, an antigen binding polypeptide or an antigen binding polypeptide complex provided herein (e.g., an antibody or antigen binding fragment thereof) is monospecific, bispecific, trispecific, or tetraspecific.

In another aspect, an antigen binding polypeptide or antigen binding polypeptide complex provided herein potently inhibits one or more SARS-CoV-2 virus variants.

In some aspects, provided herein are antibodies or antigen binding fragments thereof (e.g., monospecific or multispecific antibodies or antigen binding fragments thereof) comprising an antigen binding polypeptide having a structure represented by:
VL1-L1-VL2-L2-VH2-L3-VH1;
VL1-L1-VH2-L2-VL2-L3-VH1;
VH1-L1-VH2-L2-VL2-L3-VL1; or
VH1-L1-VL2-L2-VH2-L3-VL1;
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein; and
L1-L3 are optional amino acid linkers.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069; the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069; the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; and the VH2 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, at least one of VL1 or VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, at least one of VH1 or VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2, comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and at least one of VH1 or VH2 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, at least one of VL1 or VL2 comprises an amino acid sequence that is at least 65%, at least 70%, at least 75%, at least 80%, at least 85%, at least 90%, at least 95%, at least 99%, or 100% identical to SEQ ID NO: 147.

In some aspects, at least one of VH1 or VH2 comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) at least one of VL1 or VL2 comprises an amino acid sequence that is at least 65%, at least 70%, at least 75%, at least 80%, at least 85%, at least 90%, at least 95%, at least 99%, or 100% identical to SEQ ID NO: 147, and (ii) at least one of VH1 or VH2 comprises an amino acid sequence that is at least 65%, at least 70%, at least 75%, at least 80%, at least 85%, at least 90%, at least 95%, at least 99%, or 100% identical to SEQ ID NO: 151.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; the VH1 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; the VL2 comprises (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and the VH2 comprises (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, (i) the VL1 comprises an amino acid sequence that is at least 65%, at least 70%, at least 75%, at least 80%, at least 85%, at least 90%, at least 95%, at least 99%, or 100% identical to SEQ ID NO: 147; (ii) the VH1 comprises an amino acid sequence that is at least 65%, at least 70%, at least 75%, at least 80%, at least 85%, at least 90%, at least 95%, at least 99%, or 100% identical to SEQ ID NO: 151; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; the VH1 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054; the VL2 comprises (vii) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (viii) a CDR2 having an amino acid sequence at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (ix) a CDR3 having an amino acid sequence at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and the VH2 comprises (x) a CDR1 having an amino acid sequence at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (xi) a CDR2 having an amino acid sequence at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; (xii) a CDR3 having an amino acid sequence at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, (i) the VL1 comprises an amino acid sequence that is at least 65%, at least 70%, at least 75%, at least 80%, at least 85%, at least 90%, at least 95%, at least 99%, or 100% identical to SEQ ID NO: 147; (ii) the VH1 comprises an amino acid sequence that is at least 65%, at least 70%, at least 75%, at least 80%, at least 85%, at least 90%, at least 95%, at least 99%, or 100% identical to SEQ ID NO: 151; (iii) the VL2 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (iv) the VH2 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the antigen binding polypeptides comprised in the antibodies or antigen binding fragments thereof (e.g., monospecific or multispecific antibodies or antigen binding fragments thereof) provided herein further comprise an Fc region. In some aspects, the antigen binding polypeptides comprised in the antibodies or antigen binding fragments thereof (e.g., monospecific or multispecific antibodies or antigen binding fragments thereof) provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides are IgG1 or IgG4 antibodies or antigen binding fragments thereof.

In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides comprised in the antibodies or antigen binding fragments thereof (e.g., monospecific or multispecific antibodies or antigen binding fragments thereof) disclosed herein comprise one or more Fc effector function knockout mutation. In some aspects, the antigen binding polypeptides comprised in the antibodies or antigen binding fragments thereof (e.g., monospecific or multispecific antibodies or antigen binding fragments thereof) disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides comprised in the antibodies or antigen binding fragments thereof (e.g., monospecific or multispecific antibodies or antigen binding fragments thereof) disclosed herein comprise a mutation at a glycosylation site. In some aspects, the mutation at a glycosylation site is a mutation at an N-glycosylation site. In some aspects, the antigen binding polypeptides comprised in the antibodies or antigen binding fragments thereof (e.g., monospecific or multispecific antibodies or antigen binding fragments thereof) disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides comprised in the antibodies or antigen binding fragments thereof (e.g., monospecific or multispecific antibodies or antigen binding fragments thereof) disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between, L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptides comprised in the antibodies or antigen binding fragments thereof (e.g., monospecific or multispecific antibodies or antigen binding fragments thereof) disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides comprised in the antibodies or antigen binding fragments thereof (e.g., monospecific or multispecific antibodies or antigen binding fragments thereof) disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides comprised in the antibodies or antigen binding fragments thereof (e.g., monospecific or multispecific antibodies or antigen binding fragments thereof) provided herein further comprise an Fc region. In some aspects, the antigen binding polypeptides comprised in the antibodies or antigen binding fragments thereof (e.g., monospecific or multispecific antibodies or antigen binding fragments thereof) provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, provided herein are antibodies or antigen binding fragments thereof (e.g., monospecific or multispecific antibodies or antigen binding fragments thereof) comprising an antigen binding polypeptide having a structure represented by:
VL1-L1-VH1 or VH1-L1-VL1;
wherein:
VL1 is a first immunoglobulin light chain variable region that specifically binds to a severe acute respiratory syndrome coronavirus 2 (SARS-CoV-2) protein;
VH1 is a first immunoglobulin heavy chain variable region that specifically binds to a SARS-CoV-2 protein; and
L1 is an optional amino acid linker.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, 878, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324.627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 65, 72, 145, 152, 324, 627, 138, 172, 1045, 1050, 1055, 1060, and 1065; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 1046, 1051, 1056, and 1061, or at least 65%, at least 70%, at least 75%, at least 80%, at least 85%, at least 90%, at least 95%, at least 99%, or 100% identical to any one of amino acid sequences GAS, LGS, GTN, SAS, SDS, AAS, DAS, and DNT; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 66, 73, 146, 153, 325, 326, 764, 766, 139, 856, 173, 792, and 1066; and the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 68, 75, 148, 155, 629, 141, 175, 1047, 1052, 1057, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 19, 27, 35, 69, 76, 149, 156, 630, 142, 176, 1048, 1053, 1058, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 70, 77, 327, 150, 157, 332, 631, 765, 767, 143, 857, 177, 793, 1049, 1054, 1059, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 67, 74, 147, 154, 140, and 174; and (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 71, 78, 151, 158, 626, 144, and 178.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066.

In some aspects, the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 41, 48, 138, 145, 152, 159, 166, 172, 192, 770, 778, 785, 794, 801, 807, 814, 827, 836, 849, 858, 865, 878, 1045, 1050, 1060, and 1065; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, GAS, DAS, NNN, SDS, SYN, WAS, and DAT, or at least 65%, at least 70%, at least 75%, at least 80%, at least 85%, at least 90%, at least 95%, at least 99%, or 100% identical to any one of SEQ ID NOs: 1046, 1051, and 1061; (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 42, 139, 146, 153, 160, 167, 173, 764, 766, 768, 771, 779, 786, 792, 795, 808, 815, 821, 830, 837, 843, 850, 856, 859, 866, 872, 879, 885, and 1066; and the VH1 comprises (i) a CDR1 having an amino acid sequence that is at least 65%, at least 70%, at least 75%, at least 80%, at least 85%, at least 90%, at least 95%, at least 99%, or 100% identical to any one of SEQ ID NOs: 44, 133, 141, 148, 155, 162, 175, 773, 781, 788, 797, 803, 810, 817, 823, 832, 839, 845, 852, 861, 868, 874, 881, 1047, 1052, 1062, and 1067; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 6, 142, 149, 156, 163, 169, 176, 776, 782, 789, 798, 804, 811, 818, 824, 833, 840, 846, 853, 862, 869, 875, 882, 1048, 1053, 1063, and 1068; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 46, 143, 150, 157, 164, 170, 177, 765, 767, 769, 774, 783, 790, 793, 799, 805, 812, 819, 825, 834, 841, 847, 854, 857, 863, 870, 876, 883, 886, 1049, 1054, 1064, and 1069.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, (i) the VL1 comprises an amino acid sequence that is at least 65%, at least 70%, at least 75%, at least 80%, at least 85%, at least 90%, at least 95%, at least 99%, or 100% identical to any one of SEQ ID NOs: 43, 140, 147, 154, 161, 168, 174, 772, 780, 787, 796, 802, 809, 816, 822, 831, 838, 844, 851, 860, 867, 873, and 880; and (ii) the VH1 comprises an amino acid sequence that is at least 65%, at least 70%, at least 75%, at least 80%, at least 85%, at least 90%, at least 95%, at least 99%, or 100% identical to any one of SEQ ID NOs: 144, 151, 158, 165, 171, 178, 775, 777, 784, 791, 800, 806, 813, 820, 826, 835, 842, 848, 855, 864, 871, 877, and 884.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (ii) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (iii) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 145 or 1050; (ii) a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence that is at least 65%, at least 70%, at least 75%, at least 80%, at least 85%, at least 90%, at least 95%, at least 99%, or 100% identical to amino acid sequence AAS; and (iii) a CDR3 having an amino acid sequence that is at least 65%, at least 70%, at least 75%, at least 80%, at least 85%, at least 90%, at least 95%, at least 99%, or 100% identical to SEQ ID NO: 146 or 764; and the VH1 comprises (iv) a complementarity determining region (CDR) 1 having an amino acid sequence that is at least 65%, at least 70%, at least 75%, at least 80%, at least 85%, at least 90%, at least 95%, at least 99%, or 100% identical to SEQ ID NO: 148 or 1052; (v) a CDR2 having an amino acid sequence that is at least 65%, at least 70%, at least 75%, at least 80%, at least 85%, at least 90%, at least 95%, at least 99%, or 100% identical to SEQ ID NO: 149 or 1053; and (vi) a CDR3 having an amino acid sequence that is at least 65%, at least 70%, at least 75%, at least 80%, at least 85%, at least 90%, at least 95%, at least 99%, or 100% identical to SEQ ID NO: 150, 765, or 1054.

In some aspects, the VL1 comprises an amino acid sequence that is at least 65%, at least 70%, at least 75%, at least 80%, at least 85%, at least 90%, at least 95%, at least 99%, or 100% identical to SEQ ID NO: 147.

In some aspects, the VH1 comprises an amino acid sequence that is at least 65%, at least 70%, at least 75%, at least 80%, at least 85%, at least 90%, at least 95%, at least 99%, or 100% identical to SEQ ID NO: 151.

In some aspects, (i) the VL1 comprises an amino acid sequence that is at least 65%, at least 70%, at least 75%, at least 80%, at least 85%, at least 90%, at least 95%, at least 99%, or 100% identical to SEQ ID NO: 147, and (ii) the VH1 comprises an amino acid sequence that is at least 65%, at least 70%, at least 75%, at least 80%, at least 85%, at least 90%, at least 95%, at least 99%, or 100% identical to SEQ ID NO: 151.

In some aspects, the antigen binding polypeptides comprised in the antibodies or antigen binding fragments thereof (e.g., monospecific or multispecific antibodies or antigen binding fragments thereof) provided herein further comprise an Fc region. In some aspects, the antigen binding polypeptides comprised in the antibodies or antigen binding fragments thereof (e.g., monospecific or multispecific antibodies or antigen binding fragments thereof) provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 80%, at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides comprised in the antibodies or antigen binding fragments thereof (e.g., monospecific or multispecific antibodies or antigen binding fragments thereof) disclosed herein comprise one or more Fc effector function knockout mutation. In some aspects, the antigen binding polypeptides comprised in the antibodies or antigen binding fragments thereof (e.g., monospecific or multispecific antibodies or antigen binding fragments thereof) disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides comprised in the antibodies or antigen binding fragments thereof (e.g., monospecific or multispecific antibodies or antigen binding fragments thereof) disclosed herein comprise a mutation at a glycosylation site. In some aspects, the mutation at a glycosylation site is a mutation at an N-glycosylation site. In some aspects, the antigen binding polypeptides comprised in the antibodies or antigen binding fragments thereof (e.g., monospecific or multispecific antibodies or antigen binding fragments thereof) disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides comprised in the antibodies or antigen binding fragments thereof (e.g., monospecific or multispecific antibodies or antigen binding fragments thereof) disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptides comprised in the antibodies or antigen binding fragments thereof (e.g., monospecific or multispecific antibodies or antigen binding fragments thereof) disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides comprised in the antibodies or antigen binding fragments thereof (e.g., monospecific or multispecific antibodies or antigen binding fragments thereof) disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides comprised in the antibodies or antigen binding fragments thereof (e.g., monospecific or multispecific antibodies or antigen binding fragments thereof) provided herein further comprise an Fc region. In some aspects, the antigen binding polypeptides comprised in the antibodies or antigen binding fragments thereof (e.g., monospecific or multispecific antibodies or antigen binding fragments thereof) provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, any of the antigen binding polypeptides, antigen binding polypeptide complexes, or antibodies or antigen binding fragments thereof disclosed herein specifically binds to a sarbecovirus antigen. In some aspects, any of the antigen binding polypeptides, antigen binding polypeptide complexes, or antibodies or antigen binding fragments thereof disclosed herein specifically binds to a sarbecovirus protein. In some aspects, the sarbecovirus is a SARS-CoV-2 virus, a SARS-CoV-1 virus, a bat virus, or a pangolin virus. In some aspects, the sarbecovirus is a clade 1a sarbecovirus. In some aspects, the sarbecovirus is a clade 1b sarbecovirus. In some aspects, the sarbecovirus is a clade 2 sarbecovirus. In some aspects, the sarbecovirus is a clade 3 sarbecovirus. In some aspects, the sarbecovirus is a pangolin sarbecovirus (e.g., a Pangolin-GD virus, a Pangolin_GX-P5L virus, or a Pangolin_GX-P2V virus). In some aspects, the sarbecovirus is a bat sarbecovirus (e.g., a RaTG13-d21aa virus, a SHC014 virus, or a WIV1 virus). In some aspects, the sarbecovirus is Bat Cov W1V1, Bat Cov LYRall, Bat Cov Rs4231, or Bat Cov RSSHC014. In some aspects, the sarbecovirus is a SARS-CoV-1 sarbecovirus. In some aspects, the sarbecovirus is a SARS-CoV-2 sarbecovirus. In some aspects, the SARS-CoV-2 virus is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an Iota variant (e.g., B.1.526), a Lambda variant (e.g., C.37), an Epsilon variant (e.g., B.1.429, B.1.427, or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, or XBB.2.3.2), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof.

In some aspects, any of the antigen binding polypeptides, antigen binding polypeptide complexes, or antibodies or antigen binding fragments thereof disclosed herein comprises:
a VL at least 65%, at least 70%, at least 75%, at least 80%, at least 85%, at least 90%, at least 95%, at least 99%, or 100% identical to SEQ ID NO: 4 and a VH at least 65%, at least 70%, at least 75%, at least 80%, at least 85%, at least 90%, at least 95%, at least 99%, or 100% identical to SEQ ID NO: 8;
a VL at least 65%, at least 70%, at least 75%, at least 80%, at least 85%, at least 90%, at least 95%, at least 99%, or 100% identical to SEQ ID NO: 11 and a VH at least 65%, at least 70%, at least 75%, at least 80%, at least 85%, at least 90%, at least 95%, at least 99%, or 100% identical to SEQ ID NO: 14;
a VL at least 65%, at least 70%, at least 75%, at least 80%, at least 85%, at least 90%, at least 95%, at least 99%, or 100% identical to SEQ ID NO: 17 and a VH at least 65%, at least 70%, at least 75%, at least 80%, at least 85%, at least 90%, at least 95%, at least 99%, or 100% identical to SEQ ID NO: 21;
a VL at least 65%, at least 70%, at least 75%, at least 80%, at least 85%, at least 90%, at least 95%, at least 99%, or 100% identical to SEQ ID NO: 17 and a VH at least 65%, at least 70%, at least 75%, at least 80%, at least 85%, at least 90%, at least 95%, at least 99%, or 100% identical to SEQ ID NO: 22;
a VL at least 65%, at least 70%, at least 75%, at least 80%, at least 85%, at least 90%, at least 95%, at least 99%, or 100% identical to SEQ ID NO: 25 and a VH at least 65%, at least 70%, at least 75%, at least 80%, at least 85%, at least 90%, at least 95%, at least 99%, or 100% identical to SEQ ID NO: 29;
a VL at least 65%, at least 70%, at least 75%, at least 80%, at least 85%, at least 90%, at least 95%, at least 99%, or 100% identical to SEQ ID NO: 33 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 39 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 43 and a VH at least 65%, at least 70%, at least 75%, at least 80%, at least 85%, at least 90%, at least 95%, at least 99%, or 100% identical to SEQ ID NO: 47;
a VL at least 65%, at least 70%, at least 75%, at least 80%, at least 85%, at least 90%, at least 95%, at least 99%, or 100% identical to SEQ ID NO: 988 and a VH at least 65%, at least 70%, at least 75%, at least 80%, at least 85%, at least 90%, at least 95%, at least 99%, or 100% identical to SEQ ID NO: 57;
a VL at least 65%, at least 70%, at least 75%, at least 80%, at least 85%, at least 90%, at least 95%, at least 99%, or 100% identical to SEQ ID NO: 50 and a VH at least 65%, at least 70%, at least 75%, at least 80%, at least 85%, at least 90%, at least 95%, at least 99%, or 100% identical to SEQ ID NO: 54;
a VL at least 65%, at least 70%, at least 75%, at least 80%, at least 85%, at least 90%, at least 95%, at least 99%, or 100% identical to SEQ ID NO: 60 and a VH at least 65%, at least 70%, at least 75%, at least 80%, at least 85%, at least 90%, at least 95%, at least 99%, or 100% identical to SEQ ID NO: 64;
a VL at least 65%, at least 70%, at least 75%, at least 80%, at least 85%, at least 90%, at least 95%, at least 99%, or 100% identical to SEQ ID NO: 67 and a VH at least 65%, at least 70%, at least 75%, at least 80%, at least 85%, at least 90%, at least 95%, at least 99%, or 100% identical to SEQ ID NO: 71;
a VL at least 65%, at least 70%, at least 75%, at least 80%, at least 85%, at least 90%, at least 95%, at least 99%, or 100% identical to SEQ ID NO: 67 and a VH at least 65%, at least 70%, at least 75%, at least 80%, at least 85%, at least 90%, at least 95%, at least 99%, or 100% identical to SEQ ID NO: 626;
a VL at least 65%, at least 70%, at least 75%, at least 80%, at least 85%, at least 90%, at least 95%, at least 99%, or 100% identical to SEQ ID NO: 74 and a VH at least 65%, at least 70%, at least 75%, at least 80%, at least 85%, at least 90%, at least 95%, at least 99%, or 100% identical to SEQ ID NO: 78;
a VL at least 65%, at least 70%, at least 75%, at least 80%, at least 85%, at least 90%, at least 95%, at least 99%, or 100% identical to SEQ ID NO: 81 and a VH at least 65%, at least 70%, at least 75%, at least 80%, at least 85%, at least 90%, at least 95%, at least 99%, or 100% identical to SEQ ID NO: 85;
a VL at least 65%, at least 70%, at least 75%, at least 80%, at least 85%, at least 90%, at least 95%, at least 99%, or 100% identical to SEQ ID NO: 88 and a VH at least 65%, at least 70%, at least 75%, at least 80%, at least 85%, at least 90%, at least 95%, at least 99%, or 100% identical to SEQ ID NO: 92;
a VL at least 65%, at least 70%, at least 75%, at least 80%, at least 85%, at least 90%, at least 95%, at least 99%, or 100% identical to SEQ ID NO: 95 and a VH at least 65%, at least 70%, at least 75%, at least 80%, at least 85%, at least 90%, at least 95%, at least 99%, or 100% identical to SEQ ID NO: 98;
a VL at least 65%, at least 70%, at least 75%, at least 80%, at least 85%, at least 90%, at least 95%, at least 99%, or 100% identical to SEQ ID NO: 101 and a VH at least 65%, at least 70%, at least 75%, at least 80%, at least 85%, at least 90%, at least 95%, at least 99%, or 100% identical to SEQ ID NO: 104;
a VL at least 65%, at least 70%, at least 75%, at least 80%, at least 85%, at least 90%, at least 95%, at least 99%, or 100% identical to SEQ ID NO: 107 and a VH at least 65%, at least 70%, at least 75%, at least 80%, at least 85%, at least 90%, at least 95%, at least 99%, or 100% identical to SEQ ID NO: 111;
a VL at least 65%, at least 70%, at least 75%, at least 80%, at least 85%, at least 90%, at least 95%, at least 99%, or 100% identical to SEQ ID NO: 114 and a VH at least 65%, at least 70%, at least 75%, at least 80%, at least 85%, at least 90%, at least 95%, at least 99%, or 100% identical to SEQ ID NO: 118;
a VL at least 65%, at least 70%, at least 75%, at least 80%, at least 85%, at least 90%, at least 95%, at least 99%, or 100% identical to SEQ ID NO: 121 and a VH at least 65%, at least 70%, at least 75%, at least 80%, at least 85%, at least 90%, at least 95%, at least 99%, or 100% identical to SEQ ID NO: 125;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 137;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 132 and a VH at least 65%, at least 70%, at least 75%, at least 80%, at least 85%, at least 90%, at least 95%, at least 99%, or 100% identical to SEQ ID NO: 136;
a VL at least 65%, at least 70%, at least 75%, at least 80%, at least 85%, at least 90%, at least 95%, at least 99%, or 100% identical to SEQ ID NO: 140 and a VH at least 65%, at least 70%, at least 75%, at least 80%, at least 85%, at least 90%, at least 95%, at least 99%, or 100% identical to SEQ ID NO: 144;
a VL at least 65%, at least 70%, at least 75%, at least 80%, at least 85%, at least 90%, at least 95%, at least 99%, or 100% identical to SEQ ID NO: 147 and a VH at least 65%, at least 70%, at least 75%, at least 80%, at least 85%, at least 90%, at least 95%, at least 99%, or 100% identical to SEQ ID NO: 151;
a VL at least 65%, at least 70%, at least 75%, at least 80%, at least 85%, at least 90%, at least 95%, at least 99%, or 100% identical to SEQ ID NO: 154 and a VH at least 65%, at least 70%, at least 75%, at least 80%, at least 85%, at least 90%, at least 95%, at least 99%, or 100% identical to SEQ ID NO: 158;
a VL at least 65%, at least 70%, at least 75%, at least 80%, at least 85%, at least 90%, at least 95%, at least 99%, or 100% identical to SEQ ID NO: 161 and a VH at least 65%, at least 70%, at least 75%, at least 80%, at least 85%, at least 90%, at least 95%, at least 99%, or 100% identical to SEQ ID NO: 165;
a VL at least 65%, at least 70%, at least 75%, at least 80%, at least 85%, at least 90%, at least 95%, at least 99%, or 100% identical to SEQ ID NO: 168 and a VH at least 65%, at least 70%, at least 75%, at least 80%, at least 85%, at least 90%, at least 95%, at least 99%, or 100% identical to SEQ ID NO: 171;
a VL at least 65%, at least 70%, at least 75%, at least 80%, at least 85%, at least 90%, at least 95%, at least 99%, or 100% identical to SEQ ID NO: 174 and a VH at least 65%, at least 70%, at least 75%, at least 80%, at least 85%, at least 90%, at least 95%, at least 99%, or 100% identical to SEQ ID NO: 178;
a VL at least 65%, at least 70%, at least 75%, at least 80%, at least 85%, at least 90%, at least 95%, at least 99%, or 100% identical to SEQ ID NO: 181 and a VH at least 65%, at least 70%, at least 75%, at least 80%, at least 85%, at least 90%, at least 95%, at least 99%, or 100% identical to SEQ ID NO: 185;
a VL at least 65%, at least 70%, at least 75%, at least 80%, at least 85%, at least 90%, at least 95%, at least 99%, or 100% identical to SEQ ID NO: 188 and a VH at least 65%, at least 70%, at least 75%, at least 80%, at least 85%, at least 90%, at least 95%, at least 99%, or 100% identical to SEQ ID NO: 191;
a VL at least 65%, at least 70%, at least 75%, at least 80%, at least 85%, at least 90%, at least 95%, at least 99%, or 100% identical to SEQ ID NO: 194 and a VH at least 65%, at least 70%, at least 75%, at least 80%, at least 85%, at least 90%, at least 95%, at least 99%, or 100% identical to SEQ ID NO: 198;
a VL at least 65%, at least 70%, at least 75%, at least 80%, at least 85%, at least 90%, at least 95%, at least 99%, or 100% identical to SEQ ID NO: 201 and a VH at least 65%, at least 70%, at least 75%, at least 80%, at least 85%, at least 90%, at least 95%, at least 99%, or 100% identical to SEQ ID NO: 204;
a VL at least 65%, at least 70%, at least 75%, at least 80%, at least 85%, at least 90%, at least 95%, at least 99%, or 100% identical to SEQ ID NO: 207 and a VH at least 65%, at least 70%, at least 75%, at least 80%, at least 85%, at least 90%, at least 95%, at least 99%, or 100% identical to SEQ ID NO: 211;
a VL at least 65%, at least 70%, at least 75%, at least 80%, at least 85%, at least 90%, at least 95%, at least 99%, or 100% identical to SEQ ID NO: 214 and a VH at least 65%, at least 70%, at least 75%, at least 80%, at least 85%, at least 90%, at least 95%, at least 99%, or 100% identical to SEQ ID NO: 218;
a VL at least 65%, at least 70%, at least 75%, at least 80%, at least 85%, at least 90%, at least 95%, at least 99%, or 100% identical to SEQ ID NO: 221 and a VH at least 65%, at least 70%, at least 75%, at least 80%, at least 85%, at least 90%, at least 95%, at least 99%, or 100% identical to SEQ ID NO: 225;
a VL at least 65%, at least 70%, at least 75%, at least 80%, at least 85%, at least 90%, at least 95%, at least 99%, or 100% identical to SEQ ID NO: 228 and a VH at least 65%, at least 70%, at least 75%, at least 80%, at least 85%, at least 90%, at least 95%, at least 99%, or 100% identical to SEQ ID NO: 231;
a VL at least 65%, at least 70%, at least 75%, at least 80%, at least 85%, at least 90%, at least 95%, at least 99%, or 100% identical to SEQ ID NO: 234 and a VH at least 65%, at least 70%, at least 75%, at least 80%, at least 85%, at least 90%, at least 95%, at least 99%, or 100% identical to SEQ ID NO: 238;
a VL at least 65%, at least 70%, at least 75%, at least 80%, at least 85%, at least 90%, at least 95%, at least 99%, or 100% identical to SEQ ID NO: 240 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 243 and a VH at least 65%, at least 70%, at least 75%, at least 80%, at least 85%, at least 90%, at least 95%, at least 99%, or 100% identical to SEQ ID NO: 245;
a VL at least 65%, at least 70%, at least 75%, at least 80%, at least 85%, at least 90%, at least 95%, at least 99%, or 100% identical to SEQ ID NO: 247 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 250 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 251 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 252 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 33 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 255 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 255 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 255 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 258;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 260;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 262;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 262;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 266;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 268;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 270;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 331;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 272;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 275;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 277;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 279;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 280;
a VL at least 65%, at least 70%, at least 75%, at least 80%, at least 85%, at least 90%, at least 95%, at least 99%, or 100% identical to SEQ ID NO: 127 and a VH at least 65%, at least 70%, at least 75%, at least 80%, at least 85%, at least 90%, at least 95%, at least 99%, or 100% identical to SEQ ID NO: 282;
a VL at least 65%, at least 70%, at least 75%, at least 80%, at least 85%, at least 90%, at least 95%, at least 99%, or 100% identical to SEQ ID NO: 284 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 323 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 286 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 289 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 291 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 293 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 295 and a VH at least 65%, at least 70%, at least 75%, at least 80%, at least 85%, at least 90%, at least 95%, at least 99%, or 100% identical to SEQ ID NO: 129;
a VL at least 65%, at least 70%, at least 75%, at least 80%, at least 85%, at least 90%, at least 95%, at least 99%, or 100% identical to SEQ ID NO: 247 and a VH at least 65%, at least 70%, at least 75%, at least 80%, at least 85%, at least 90%, at least 95%, at least 99%, or 100% identical to SEQ ID NO: 282;
a VL at least 65%, at least 70%, at least 75%, at least 80%, at least 85%, at least 90%, at least 95%, at least 99%, or 100% identical to SEQ ID NO: 250 and a VH at least 65%, at least 70%, at least 75%, at least 80%, at least 85%, at least 90%, at least 95%, at least 99%, or 100% identical to SEQ ID NO: 282;
a VL at least 65%, at least 70%, at least 75%, at least 80%, at least 85%, at least 90%, at least 95%, at least 99%, or 100% identical to SEQ ID NO: 39 and a VH at least 65%, at least 70%, at least 75%, at least 80%, at least 85%, at least 90%, at least 95%, at least 99%, or 100% identical to SEQ ID NO: 298;
a VL at least 65%, at least 70%, at least 75%, at least 80%, at least 85%, at least 90%, at least 95%, at least 99%, or 100% identical to SEQ ID NO: 39 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 299 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 300 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 302 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 303 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 305 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 307 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 308 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 310 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 312 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 314 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 315 and a VH at least 65%, at least 70%, at least 75%, at least 80%, at least 85%, at least 90%, at least 95%, at least 99%, or 100% identical to SEQ ID NO: 37;
a VL at least 65%, at least 70%, at least 75%, at least 80%, at least 85%, at least 90%, at least 95%, at least 99%, or 100% identical to SEQ ID NO: 305 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 251 and a VH at least 65%, at least 70%, at least 75%, at least 80%, at least 85%, at least 90%, at least 95%, at least 99%, or 100% identical to SEQ ID NO: 254;
a VL at least 65%, at least 70%, at least 75%, at least 80%, at least 85%, at least 90%, at least 95%, at least 99%, or 100% identical to SEQ ID NO: 255 and a VH at least 65%, at least 70%, at least 75%, at least 80%, at least 85%, at least 90%, at least 95%, at least 99%, or 100% identical to SEQ ID NO: 282;
a VL at least 65%, at least 70%, at least 75%, at least 80%, at least 85%, at least 90%, at least 95%, at least 99%, or 100% identical to SEQ ID NO: 317 and a VH at least 65%, at least 70%, at least 75%, at least 80%, at least 85%, at least 90%, at least 95%, at least 99%, or 100% identical to SEQ ID NO: 321;
a VL at least 65%, at least 70%, at least 75%, at least 80%, at least 85%, at least 90%, at least 95%, at least 99%, or 100% identical to SEQ ID NO: 772 and a VH at least 65%, at least 70%, at least 75%, at least 80%, at least 85%, at least 90%, at least 95%, at least 99%, or 100% identical to SEQ ID NO: 775;
a VL at least 65%, at least 70%, at least 75%, at least 80%, at least 85%, at least 90%, at least 95%, at least 99%, or 100% identical to SEQ ID NO: 43 and a VH at least 65%, at least 70%, at least 75%, at least 80%, at least 85%, at least 90%, at least 95%, at least 99%, or 100% identical to SEQ ID NO: 777;
a VL at least 65%, at least 70%, at least 75%, at least 80%, at least 85%, at least 90%, at least 95%, at least 99%, or 100% identical to SEQ ID NO: 780 and a VH at least 65%, at least 70%, at least 75%, at least 80%, at least 85%, at least 90%, at least 95%, at least 99%, or 100% identical to SEQ ID NO: 784;
a VL at least 65%, at least 70%, at least 75%, at least 80%, at least 85%, at least 90%, at least 95%, at least 99%, or 100% identical to SEQ ID NO: 787 and a VH at least 65%, at least 70%, at least 75%, at least 80%, at least 85%, at least 90%, at least 95%, at least 99%, or 100% identical to SEQ ID NO: 791;
a VL at least 65%, at least 70%, at least 75%, at least 80%, at least 85%, at least 90%, at least 95%, at least 99%, or 100% identical to SEQ ID NO: 796 and a VH at least 65%, at least 70%, at least 75%, at least 80%, at least 85%, at least 90%, at least 95%, at least 99%, or 100% identical to SEQ ID NO: 800;
a VL at least 65%, at least 70%, at least 75%, at least 80%, at least 85%, at least 90%, at least 95%, at least 99%, or 100% identical to SEQ ID NO: 802 and a VH at least 65%, at least 70%, at least 75%, at least 80%, at least 85%, at least 90%, at least 95%, at least 99%, or 100% identical to SEQ ID NO: 806;
a VL at least 65%, at least 70%, at least 75%, at least 80%, at least 85%, at least 90%, at least 95%, at least 99%, or 100% identical to SEQ ID NO: 809 and a VH at least 65%, at least 70%, at least 75%, at least 80%, at least 85%, at least 90%, at least 95%, at least 99%, or 100% identical to SEQ ID NO: 813;
a VL at least 65%, at least 70%, at least 75%, at least 80%, at least 85%, at least 90%, at least 95%, at least 99%, or 100% identical to SEQ ID NO: 816 and a VH at least 65%, at least 70%, at least 75%, at least 80%, at least 85%, at least 90%, at least 95%, at least 99%, or 100% identical to SEQ ID NO: 820;
a VL at least 65%, at least 70%, at least 75%, at least 80%, at least 85%, at least 90%, at least 95%, at least 99%, or 100% identical to SEQ ID NO: 822 and a VH at least 65%, at least 70%, at least 75%, at least 80%, at least 85%, at least 90%, at least 95%, at least 99%, or 100% identical to SEQ ID NO: 826;
a VL at least 65%, at least 70%, at least 75%, at least 80%, at least 85%, at least 90%, at least 95%, at least 99%, or 100% identical to SEQ ID NO: 831 and a VH at least 65%, at least 70%, at least 75%, at least 80%, at least 85%, at least 90%, at least 95%, at least 99%, or 100% identical to SEQ ID NO: 835;
a VL at least 65%, at least 70%, at least 75%, at least 80%, at least 85%, at least 90%, at least 95%, at least 99%, or 100% identical to SEQ ID NO: 838 and a VH at least 65%, at least 70%, at least 75%, at least 80%, at least 85%, at least 90%, at least 95%, at least 99%, or 100% identical to SEQ ID NO: 842;
a VL at least 65%, at least 70%, at least 75%, at least 80%, at least 85%, at least 90%, at least 95%, at least 99%, or 100% identical to SEQ ID NO: 844 and a VH at least 65%, at least 70%, at least 75%, at least 80%, at least 85%, at least 90%, at least 95%, at least 99%, or 100% identical to SEQ ID NO: 848;
a VL at least 65%, at least 70%, at least 75%, at least 80%, at least 85%, at least 90%, at least 95%, at least 99%, or 100% identical to SEQ ID NO: 851 and a VH at least 65%, at least 70%, at least 75%, at least 80%, at least 85%, at least 90%, at least 95%, at least 99%, or 100% identical to SEQ ID NO: 855;
a VL at least 65%, at least 70%, at least 75%, at least 80%, at least 85%, at least 90%, at least 95%, at least 99%, or 100% identical to SEQ ID NO: 860 and a VH at least 65%, at least 70%, at least 75%, at least 80%, at least 85%, at least 90%, at least 95%, at least 99%, or 100% identical to SEQ ID NO: 864;
a VL at least 65%, at least 70%, at least 75%, at least 80%, at least 85%, at least 90%, at least 95%, at least 99%, or 100% identical to SEQ ID NO: 867 and a VH at least 65%, at least 70%, at least 75%, at least 80%, at least 85%, at least 90%, at least 95%, at least 99%, or 100% identical to SEQ ID NO: 871;
a VL at least 65%, at least 70%, at least 75%, at least 80%, at least 85%, at least 90%, at least 95%, at least 99%, or 100% identical to SEQ ID NO: 873 and a VH at least 65%, at least 70%, at least 75%, at least 80%, at least 85%, at least 90%, at least 95%, at least 99%, or 100% identical to SEQ ID NO: 877; or
a VL at least 65%, at least 70%, at least 75%, at least 80%, at least 85%, at least 90%, at least 95%, at least 99%, or 100% identical to SEQ ID NO: 880 and a VH at least 65%, at least 70%, at least 75%, at least 80%, at least 85%, at least 90%, at least 95%, at least 99%, or 100% identical to SEQ ID NO: 884.

In some aspects, in any one of the antigen binding polypeptides or antigen binding polypeptide complexes disclosed herein:
(i) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049;
and/or
(ii) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054; and/or
(iii) any one of the VL comprises a CDR1 having an amino acid sequence at least 65%, at least 70%, at least 75%, at least 80%, at least 85%, at least 90%, at least 95%, at least 99%, or 100% identical to SEQ ID NO: 172 or 1060, a CDR2 having an amino acid sequence at least 65%, at least 70%, at least 75%, at least 80%, at least 85%, at least 90%, at least 95%, at least 99%, or 100% identical to SEQ ID NO: 1061 or an amino acid sequence at least 65%, at least 70%, at least 75%, at least 80%, at least 85%, at least 90%, at least 95%, at least 99%, or 100% identical to amino acid sequence DAS, and a CDR3 having an amino acid sequence at least 65%, at least 70%, at least 75%, at least 80%, at least 85%, at least 90%, at least 95%, at least 99%, or 100% identical to SEQ ID NO: 173 or 792; and any one of the VH comprises a CDR1 having an amino acid sequence at least 65%, at least 70%, at least 75%, at least 80%, at least 85%, at least 90%, at least 95%, at least 99%, or 100% identical to SEQ ID NO: 175 or 1062, a CDR2 having an amino acid sequence at least 65%, at least 70%, at least 75%, at least 80%, at least 85%, at least 90%, at least 95%, at least 99%, or 100% identical to SEQ ID NO: 176 or 1063, and a CDR3 having an amino acid sequence at least 65%, at least 70%, at least 75%, at least 80%, at least 85%, at least 90%, at least 95%, at least 99%, or 100% identical to SEQ ID NO: 177, 793, or 1064.

For example, an antigen binding polypeptide may comprise one VL/VH pair, wherein the VL/VH pair comprises one of (i), (ii), or (iii); or an antigen binding polypeptide may comprise two VL/VH pairs, wherein the first VL/VH pair comprises one of (i), (ii), or (iii) and the second VL/VH pair comprises one of (i), (ii), or (iii), in any combinations (e.g., the first VL/VH pair comprise (i) and the second VL/VH pair also comprises (i); or the first VL/VH pair comprise (i) and the second VL/VH pair comprises (ii); or the first VL/VH pair comprise (iii) and the second VL/VH pair comprises (ii); etc.).

For example, an antigen binding polypeptide complex may comprise three VL/VH pairs, wherein the first VL/VH pair comprises one of (i), (ii), or (iii), the second VL/VH pair comprises one of (i), (ii), or (iii), and the third VL/VH pair comprises one of (i), (ii), or (iii), in any combination (e.g., the first VL/VH pair comprises (i), the second VL/VH pair also comprises (i), and the third VL/VH pair comprises (ii); or the first VL/VH pair comprises (ii), the second VL/VH pair comprises (i), and the third VL/VH pair comprises (iii); or the first VL/VH pair comprises (iii), the second VL/VH pair comprises (ii), and the third VL/VH pair also comprises (ii); etc.); or an antigen binding polypeptide complex may comprise four VL/VH pairs, wherein the first VL/VH pair comprises one of (i), (ii), or (iii), the second VL/VH pair comprises one of (i), (ii), or (iii), the third VL/VH pair comprises one of (i), (ii), or (iii), and the fourth VL/VH pair comprises one of (i), (ii), or (iii), in any combination (e.g., the first VL/VH pair comprises (i), the second VL/VH pair also comprises (i), the third VL/VH pair comprises (ii), and the fourth VL/VH pair also comprises (ii); or the first VL/VH pair comprises (i), the second VL/VH pair comprises (ii), the third VL/VH pair comprises (iii), and the fourth VL/VH pair also comprises (iii); or the first VL/VH pair comprises (iii), the second VL/VH pair comprises (ii), the third VL/VH pair comprises (i), and the fourth VL/VH pair comprises (iii); etc.).

In some of those aspects, in any one of the antigen binding polypeptides or antigen binding polypeptide complexes disclosed herein:
(i) any one of the VL comprises an amino acid sequence that is at least 65%, at least 70%, at least 75%, at least 80%, at least 85%, at least 90%, at least 95%, at least 99%, or 100% identical to SEQ ID NO: 154; and any one of the VH comprises an amino acid sequence that is at least 65%, at least 70%, at least 75%, at least 80%, at least 85%, at least 90%, at least 95%, at least 99%, or 100% identical to SEQ ID NO: 158; and/or
(ii) any one of the VL comprises an amino acid sequence that is at least 65%, at least 70%, at least 75%, at least 80%, at least 85%, at least 90%, at least 95%, at least 99%, or 100% identical to SEQ ID NO: 147; and any one of the VH comprises an amino acid sequence that is at least 65%, at least 70%, at least 75%, at least 80%, at least 85%, at least 90%, at least 95%, at least 99%, or 100% identical to SEQ ID NO: 151; and/or
(iii) any one of the VL comprises an amino acid sequence that is at least 65%, at least 70%, at least 75%, at least 80%, at least 85%, at least 90%, at least 95%, at least 99%, or 100% identical to SEQ ID NO: 174; and any one of the VH comprises an amino acid sequence that is at least 65%, at least 70%, at least 75%, at least 80%, at least 85%, at least 90%, at least 95%, at least 99%, or 100% identical to SEQ ID NO: 178.

For example, an antigen binding polypeptide may comprise one VL/VH pair, wherein the VL/VH pair comprises one of (i), (ii), or (iii); or an antigen binding polypeptide may comprise two VL/VH pairs, wherein the first VL/VH pair comprises one of (i), (ii), or (iii) and the second VL/VH pair comprises one of (i), (ii), or (iii), in any combinations (e.g., the first VL/VH pair comprise (i) and the second VL/VH pair also comprises (i); or the first VL/VH pair comprise (i) and the second VL/VH pair comprises (ii); or the first VL/VH pair comprise (iii) and the second VL/VH pair comprises (ii); etc.).

For example, an antigen binding polypeptide complex may comprise three VL/VH pairs, wherein the first VL/VH pair comprises one of (i), (ii), or (iii), the second VL/VH pair comprises one of (i), (ii), or (iii), and the third VL/VH pair comprises one of (i), (ii), or (iii), in any combination (e.g., the first VL/VH pair comprises (i), the second VL/VH pair also comprises (i), and the third VL/VH pair comprises (ii); or the first VL/VH pair comprises (ii), the second VL/VH pair comprises (i), and the third VL/VH pair comprises (iii); or the first VL/VH pair comprises (iii), the second VL/VH pair comprises (ii), and the third VL/VH pair also comprises (ii); etc.); or an antigen binding polypeptide complex may comprise four VL/VH pairs, wherein the first VL/VH pair comprises one of (i), (ii), or (iii), the second VL/VH pair comprises one of (i), (ii), or (iii), the third VL/VH pair comprises one of (i), (ii), or (iii), and the fourth VL/VH pair comprises one of (i), (ii), or (iii), in any combination (e.g., the first VL/VH pair comprises (i), the second VL/VH pair also comprises (i), the third VL/VH pair comprises (ii), and the fourth VL/VH pair also comprises (ii); or the first VL/VH pair comprises (i), the second VL/VH pair comprises (ii), the third VL/VH pair comprises (iii), and the fourth VL/VH pair also comprises (iii); or the first VL/VH pair comprises (iii), the second VL/VH pair comprises (ii), the third VL/VH pair comprises (i), and the fourth VL/VH pair comprises (iii); etc.).

In some aspects, any one of the antigen binding polypeptides, antigen binding polypeptide complexes, polypeptides, antibodies or antigen binding fragments thereof (e.g., anti-SARS-CoV-2 virus antibodies or antigen binding fragments thereof) disclosed herein have a poly-reactivity score of from 0 to about 100, from 0 to about 90, from 0 to about 80, from 0 to about 70, from 0 to about 60, from 0 to about 50, from 0 to about 40, from 0 to about 30, from 0 to about 25, from 0 to about 20, from 0 to about 15, from 0 to about 10, or from 0 to about 5.

In some aspects, any one of the antigen binding polypeptides, antigen binding polypeptide complexes, polypeptides, antibodies or antigen binding fragments thereof (e.g., anti-SARS-CoV-2 virus antibodies or antigen binding fragments thereof) disclosed herein have a poly-reactivity score of from 0 to about 50, from 0 to about 40, from 0 to about 30, from 0 to about 25, from 0 to about 20, from 0 to about 15, from 0 to about 10, or from 0 to about 5.

In some aspects, any one of the antigen binding polypeptides, antigen binding polypeptide complexes, polypeptides, antibodies or antigen binding fragments thereof (e.g., anti-SARS-CoV-2 virus antibodies or antigen binding fragments thereof) disclosed herein have a poly-reactivity score of from 0 to about 10, or from 0 to about 5.

In some aspects, any one of the antigen binding polypeptides, antigen binding polypeptide complexes, polypeptides, antibodies or antigen binding fragments thereof (e.g., anti-SARS-CoV-2 virus antibodies or antigen binding fragments thereof) disclosed herein have a poly-reactivity score of about 100, 95, 90, 85, 80, 75, 70, 65, 60, 55, 50, 45, 40, 35, 30, 25, 20, 19, 18, 17, 16, 15, 14, 13, 12, 10, 9, 8, 7, 6, 5, 4, 3, 2, 1, or 0.

In some aspects, any one of the antigen binding polypeptides, antigen binding polypeptide complexes, polypeptides, antibodies or antigen binding fragments thereof (e.g., anti-SARS-CoV-2 virus antibodies or antigen binding fragments thereof) disclosed herein have a poly-reactivity score of about 20, 19, 18, 17, 16, 15, 14, 13, 12, 10, 9, 8, 7, 6, 5, 4, 3, 2, 1, or 0).

In some aspects, any one of the antigen binding polypeptides, antigen binding polypeptide complexes, polypeptides, antibodies or antigen binding fragments thereof (e.g., anti-SARS-CoV-2 virus antibodies or antigen binding fragments thereof) disclosed herein have a poly-reactivity score of about 10, 9, 8, 7, 6, 5, 4, 3, 2, 1, or 0).

### Amino Acid Linkers

In some aspects, an antigen binding polypeptide or polypeptide complex (e.g., an antibody or antigen binding fragment thereof) provided herein comprises one or more optional amino acid linkers between two or more regions of the antigen binding polypeptide or polypeptide complex.

As used herein, an “amino acid linker” can be a single amino acid or short amino acid sequence that is capable of joining two regions of an antigen binding polypeptide or polypeptide complex provided herein in a stable manner that maintains or promotes a function associated with the regions. However, as used herein, an “amino acid linker” can also include where an amino acid linker is not present between two regions (referred to herein as an amino acid linker having 0 amino acids). In some aspects, an amino acid linker is represented herein in a structure of an antigen binding polypeptide or polypeptide complex by the abbreviation “1” or “L” and a number (e.g., L1 to denote a first linker, L2 to denote a second linker, L3 to denote a third linker. L4 to denote a fourth linker. L5 to denote a fifth linker. L6 to denote a sixth linker. L7 to denote a seventh linker, and L8 to denote an eighth linker. L9 to denote a ninth linker. L10 to denote a tenth linker). In some aspects, such enumerated amino acid linkers (e.g., L1) can have the same or different sequence as any other enumerated amino acid linker (e.g., L2, etc.). The terms “amino acid linker” and “linker” are used interchangeably herein.

In one aspect, an amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) has a length of from 0 amino acids (i.e., an amino acid linker is not present) to about 50 amino acids. In another aspect, the amino acid linker has a length of from 0 amino acids to about 45 amino acids, 0 amino acids to about 40 amino acids, 0 amino acids to about 35 amino acids, 0 amino acids to about 30 amino acids, 0 amino acids to about 25 amino acids, 0 amino acids to about 20 amino acids, 0 amino acids to about 15 amino acids, 0 amino acids to about 10 amino acids, 0 amino acids to about 5 amino acids, about 1 amino acid to about 45 amino acids, about 1 amino acid to about 40 amino acids, about 1 amino acid to about 35 amino acids, about 1 amino acid to about 30 amino acids, about 1 amino acid to about 25 amino acids, about 1 amino acid to about 20 amino acids, 1 amino acid to about 15 amino acids, about 1 amino acid to about 10 amino acids, about 1 amino acid to about 5 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 40 amino acids to about 50 amino acids, about 40 amino acids to about 45 amino acids, or about 45 amino acids to about 50 amino acids

In another aspect, the amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) has 0 amino acids (i.e., an amino acid linker is not present) or about 1, about 2, about 3, about 4, about 5, about 6, about 7, about 8, about 9), about 10), about 11, about 12, about 13, about 14, about 15, about 16, about 17, about 18, about 19, about 20, about 25, about 30, about 35, about 40), about 45, or about 50 amino acids.

In one aspect, the amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) consists of one or more amino acid residues. In one aspect, the amino acid residues are selected from the group consisting of glycine, alanine, serine, threonine, cysteine, asparagine, glutamine, leucine, isoleucine, valine, proline, histidine, aspartic acid, glutamic acid, lysine, arginine, methionine, phenylalanine, tryptophan, and tyrosine.

In one aspect, an amino acid linker is non-immunogenic. In one aspect, a non-immunogenic linker consists of serine, glycine and/or alanine residues, or consists of serine and/or glycine residues. In another aspect, an amino acid linker does not contain a T cell epitope or consensus T cell epitope.

In one aspect, an amino acid linker consists of one or more residues of alanine, cysteine, glycine, isoleucine, leucine, methionine, phenylalanine, proline, tryptophan, tyrosine, or valine (e.g., one or more of L1, L2, L3, L4, L5, L6, L7, L8, L9, or L10 of a first, second, third and/or fourth polypeptide of an antigen binding polypeptide or polypeptide complex structure provided herein).

Amino acid linker sequences that can be used with the antigen binding polypeptides or polypeptide complexes (e.g., an antibody or antigen binding fragment thereof) provided herein are well known and can be incorporated into the antigen binding polypeptides and polypeptide complexes provided herein using routine molecular biology and recombinant DNA techniques. See, e.g., Chen et al., Adv Drug Deliv Rev., 65(10):1357-1369, 2013; and Chichili et al., Protein Sci., 22(2):153-167, 2013.

In one aspect, an amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In one aspect, an amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971.

In some aspects, L1 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L1 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L1 comprises 0 amino acids (i.e., L1 is not present).

In some aspects, L2 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L2 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L2 comprises 0 amino acids (i.e., L2 is not present).

In some aspects, L3 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L3 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L3 comprises 0 amino acids (i.e., L3 is not present).

In some aspects, L4 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L4 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L4 comprises 0 amino acids (i.e., L4 is not present).

In some aspects, L5 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L5 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L5 comprises 0 amino acids (i.e., L5 is not present).

In some aspects, L6 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L6 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L6 comprises 0 amino acids (i.e., L6 is not present).

In some aspects, L7 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L7 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L7 comprises 0 amino acids (i.e., L7 is not present).

In some aspects, L8 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L8 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L8 comprises 0 amino acids (i.e., L8 is not present).

In some aspects, L9 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L9 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L9 comprises 0 amino acids (i.e., L9 is not present).

In some aspects, L10 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L10 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L10 comprises 0 amino acids (i.e., L10 is not present).

In some aspects, L11 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L10 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L10 comprises 0 amino acids (i.e., L11 is not present).

In some aspects, L12 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L10 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L10 comprises 0 amino acids (i.e., L12 is not present).

In some aspects, the SARS-CoV-2 variant of the SARS-CoV-2 protein (e.g., SARS-CoV-2 spike protein) to which an antigen binding polypeptide or polypeptide complex provided herein specifically binds is a variant of concern (VOC). In some aspects, the SARS-CoV-2 variant of the SARS-CoV-2 protein (e.g., SARS-CoV-2 spike protein) to which an antigen binding polypeptide or polypeptide complex provided herein specifically binds is a variant under monitoring (e.g., variant with genetic changes suspected to affect virus characteristics and some indication of posing a future risk, but further studies are needed).

In some aspects, the SARS-CoV-2 variant of the SARS-CoV-2 protein (e.g., SARS-CoV-2 spike protein) to which an antigen binding polypeptide or polypeptide complex provided herein specifically binds is a the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an Iota variant (e.g., B.1.526), a Lambda variant (e.g., C.37), an Epsilon variant (e.g., B.1.429, B.1.427 or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, or XBB.2.3.2), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof.

In some aspects, the Omicron variant is B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, KP.3.1.1, or a combination or variant thereof.

In some aspects, the SARS-CoV-2 variant of the SARS-CoV-2 protein (e.g., SARS-CoV-2 spike protein) to which an antigen binding polypeptide or polypeptide complex provided herein specifically binds is a AZ.5, C.1.2, B.1.617.1, B.1.526, B.1.525, B.1.630, B.1.640, AV.1, AT.1, R.1, B.1.466.2, B.1.1.519, C.36.3, B.1.214.2, B.1.427, B.1.429, B.1.1.523, B.1.619, B.1.620, B.1.1.207, B.1.1.317, B.1.616, B.1.618, or B.1.640.2 variant, or a combination thereof.

In other aspects, the SARS-CoV-2 variant of the SARS-CoV-2 protein (e.g., SARS-CoV-2 spike protein) to which an antigen binding polypeptide or polypeptide complex provided herein specifically binds is an Alpha (B.1.1.7), Beta (B.1.351), Gamma (P.1), Delta (B.1.617.2), or Omicron (B.1.1.529) variant, or a combination thereof.

In some aspects, the SARS-CoV-2 spike protein has an amino acid mutation of one or more of the following: F2L, L5F, L5I, V6F, L7V, P9L, S12C, S13I, Q14H, C15F, N17K, L18F, T20I, T22N, T22A, T22I, Q23K, P25S, A27S, A27V, T29I, F32L, R34C, H49Y, S50L, T51I, Q52L, Q52H, L54F, L54W, F55I, P57L, H69Y, S71F, G72V, T73I, G75V, T76I, F79L, D80N, D80Y, N87Y, D88N, D88E, D88Y, D88A, V90F, T95A, T95I, E96D, K97T, S98F, R102I, I105L, D111N, K113R, L118F, V130A, E132D, C136R, D138H, L141-, L141F, G142V, G142-, V143F, V143-, Y144-, Y144V, Y145H, H146Y, K147E, N148S, S151I, W152C, M153T, M153V, M153I, E154V, F157L, R158S, L176F, M177I, D178N, G181V, L189F, R190K, I203M, I210-, R214L, D215Y, D215G, L216F, Q218L, F220L, S221L, A222V, A222P, D228H, L229F, Q239R, T240I, L242F, A243S, A243V, H245R, H245Y, R246K, D253Y, D253G, S254F, S256L, W258L, G261V, G261R, A262S, Y265C, V267L, R273S, E281Q, A288S, L293V, D294E, P295S, E298G, T307I, V308L, E309Q, Q314K, Q314L, Q314H, T315I, Q321L, T323I, P330S, A344S, T345S, A348T, A348S, N354K, R357K, V367F, V382L, P384L, V395I, R403K, V407I, A411S, G413R, K417T, N439K, N440K, L441I, G446V, R457K, K458Q, G476S, S477N, P479L, V483A, E484Q, E484K, Q493L, S494P, L452R, Y453F, Y508H, N501Y, H519Q, A520S, A522V, K529E, G545S, T547I, L552F, T553I, E554D, K558N, A570V, T572I, D574Y, E583D, I584V, S596I, I598V, N603H, Q613H, D614G, V615F, T618A, P621S, V622F, V622I, V622A, A623S, H625Y, A626V, P631S, W633R, G639V, S640F, A647S, A647V, E654Z, E654K, H655Y, N658Y, A668S, A672V, Q675R, Q675H, T676S, T676I, Q677H, Q677R, T678I, P681L, P681R, P681H, R682W, A684S, A684T, V687L, A688V, A688S, S689I, S691F, S698L, N703D, S704L, V705F, A706V, I714M, T716I, I720V, T724A, M731I, T732A, T732I, G744V, D745G, N751D, L754F, R765S, R765H, T768I, G769A, A771S, T778I, Q779H, E780Q, A783S, D808V, D808G, P809S, I818V, L822F, D830H, D830Y, Q836P, Q836L, G838D, A845S, A845D, A845V, A845D, A845S, A846V, R847I, K854R, N856S, T859I, D867N, A879V, A879S, F888L, A893E, A893V, E918V, L922F, A924S, A924V, S929I, D936H, D936Y, L938F, S939F, T941I, G946V, A958S, N969S, L981F, T1006I, V1008T, T1009I, A1016S, A1020V, F1052L, P1053T, L1063F, V1065L, A1070V, Q1071H, E1072V, K1073N, A1078V, A1078S, G1085R, K1086N, R1091L, H1101Y, V1104L, P1112L, D1118Y, T1120I, V1122L, S1123P, G1124V, G1124C, V1129A, I1130M, T1136I, D1139H, L1141F, D1146H, S1147L, D1153Y, P1162Q, P1162S, P1162Q, P1162S, D1163Y, G1167V, D1168H, V1176F, N1187Y, K1191N, N1192T, E1195Q, L1203F, K1205N, E1207A, I219V, G1219S, I1221T, V1228L, M1229I, V1230L, T1231I, T1231A, C1235F, M1237I, M1237V, M1237T, T1238I, K1245N, C1247F, G1251R, D1259H, S1261F, P1263L, V1264L, a deletion of residues 69 and 70, a deletion of residues 246-252 and D253N, or a combination thereof.

### Modifications

In some aspects, an antigen binding polypeptide or polypeptide complex (e.g., an antibody or antigen binding fragment thereof) provided herein comprises an effector function mutation or half-life extension mutation.

Effector functions are an important part of the humoral immune response and form a link between innate and adaptive immunity. Most effector functions are induced via the Fc region of an antibody, which can interact with complement proteins and specialized Fc receptors. As used herein, an “effector function mutation” or “Fc effector function mutation” refer to a change in the amino acid sequence, typically in the Fc region, which can increase or decrease effector function, for example, increase binding affinity of Fc for specific Fc receptors, or increase antibody-dependent cellular cytotoxicity (ADCC) activity.

“Half-life” of a pharmaceutically active substance is the time it takes for the amount of the substance, once administered to the body, to reduce by half. A “half-life extension mutation” of an antigen binding polypeptide or polypeptide complex provided herein refers to a change in the amino acid sequence, typically in the Fc region, which increases the half-life of the antigen binding polypeptide or polypeptide complex (e.g., by increasing Fc receptor binding affinity, slowing off-rate for Fc and Fc receptors, and/or increased sialylation).

Examples of Fc effector function mutations that decrease or knockout function include, but are not limited to, the following substitutions in the Fc region, based on the EU numbering scheme: (LA) L234A, L235A, (LS) M428L, N434S, P239A, N297A, or equivalent thereof, or a combination thereof.

Examples of Fc effector function mutations that increase function include, but are not limited to, the following substitutions in the Fc region, based on the EU numbering scheme: S298A/E333A/K334A, S239D/I332E, S239D/A330L/I332E, G236A/S239D/I332E, or equivalent thereof, or a combination thereof.

Additional examples of effector function mutations, half-life extension mutations and methods for incorporating the same into an amino acid sequence are known and described, for example, in Saunders, “Conceptual Approaches to Modulating Antibody Effector Functions and Circulation Half-Life.” Front. Immunol. Jun. 7, 2019.

In another aspect, an antigen binding polypeptide or polypeptide complex (e.g., an antibody or antigen binding fragment thereof) provided herein comprises one or more knob-into-hole modifications.

The term “knob-into-hole modification.” as used herein, refers to a genetic modification that directs the pairing of two polypeptides to promote heterodimerization. In one aspect, the modification introduces a protuberance (knob) into one polypeptide and a cavity (hole) into the other polypeptide at an interface in which the two polypeptides interact. In another aspect, a knob-into-hole modification can be created by introducing only a hole modification, for example, by replacing an amino acid residue with a smaller side chain than the original amino acid residue (e.g., a substitution of one or more serine, threonine, valine or alanine residues, or a combination thereof). In yet another aspect, a knob-into-hole modification can be created by introducing only a knob modification, for example, by replacing an amino acid residue with a larger side chain than the original amino acid residue (e.g., a substitution of one or more tryptophan or tyrosine residues, or a combination thereof).

In one aspect, the knob-into-hole modification is in the binding interface of two Fc regions, the binding interface of two CH2 regions, the binding interface of two CH3 regions, the binding interface of a CL region and a CH1 region, or the binding interface of a VH region and a VL region. Sec, e.g., U.S. Pub. No. 2007/0178552. Int'l Pub. No, WO 96/027011. Int'l Pub. No, WO 98/050431 and Zhu et al., Protein Science 6:781-788, 1987.

In one aspect, the antigen binding polypeptide or polypeptide complex comprises one, two, three, four, five, six, seven, eight, nine, ten, or more knob-into-hole modifications.

Knob-into-hole modifications are well known and can be incorporated into antigen binding polypeptides and polypeptide complexes provided herein using routine molecular biology and recombinant DNA techniques. See, e.g., U.S. Pub. No. 2003/0078385; Int'l Pub. No, WO 96/027011; Ridgway et al., Protein Eng., 9:617-621, 1996; and Merchant et al., Nat. Biotechnol., 16:677-681, 1998.

In one aspect, the knob-into-hole modification is an amino acid substitution. As used herein, such a substitution is described based on the EU numbering scheme of Kabat, which corresponds to the numbering in the Protein Data Bank (PDB).

In one aspect, the knob-into-hole modification is a knob substitution of S354C, T366W, or equivalent thereof, or a combination thereof based on the EU numbering scheme.

In one aspect, the knob-into-hole modification is a hole substitution of Y349C, T366S, L368A, Y407V, or equivalent thereof, or a combination thereof, based on the EU numbering scheme.

In another aspect, the knob-into-hole modifications are knob substitutions of S354C, T366W, or equivalent thereof, based on the EU numbering scheme.

In another aspect, the knob-into-hole modifications are hole substitutions of Y349C, T366S, L368A and Y407V, or equivalent thereof, based on the EU numbering scheme.

In another aspect, an antigen binding polypeptide complex is an IgG1 or IgG4 antibody or antigen binding fragment thereof and the knob-into-hole modifications are knob substitutions of S354C and T366W, or equivalent thereof, and/or hole substitutions of Y349C, T366S, L368A and Y407V, or equivalent thereof.

In one aspect, the antigen binding polypeptide complex is an IgG1 or IgG4 antibody or antigen binding fragment thereof and the knob-into-hole modifications are knob substitutions of S354C and T366W, or equivalent thereof.

In one aspect, the antigen binding polypeptide complex is an IgG1 or IgG4 antibody or antigen binding fragment thereof and the knob-into-hole modifications are hole substitutions of Y349C, T366S, L368A and Y407V, or equivalent thereof.

In one aspect, the antigen binding polypeptide complex is an IgG1 or IgG4 antibody or antigen binding fragment thereof and the knob-into-hole modifications are knob substitutions of S354C and T366W, or equivalent thereof, and hole substitutions of Y349C, T366S, L368A and Y407V, or equivalent thereof.

In one aspect, the antigen binding polypeptide complex is an IgG1 or IgG4 antibody or antigen binding fragment thereof and the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In one aspect, the antigen binding polypeptide complex is an IgG1 or IgG4 antibody or antigen binding fragment thereof and the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In one aspect, the antigen binding polypeptide complex is an IgG1 or IgG4 antibody or antigen binding fragment thereof and the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

### Detectable Labels

In some aspects, an antigen binding polypeptide or polypeptide complex (e.g., an antibody or antigen binding fragment thereof) provided herein comprises one or more detectable labels. An antigen binding polypeptide or polypeptide complex (e.g., an antibody or antigen binding fragment thereof) containing a detectable label is useful in therapeutic, diagnostic, imaging (e.g., radioimaging), or basic research applications.

In one aspect, the detectable label is a radioactive label. Examples of a radioactive label include, but are not limited to, the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc, ¹⁸⁶Re and ⁸⁹Zr.

In another aspect, the detectable label is a chemiluminescent label, fluorescent label, enzyme, biotin, or a combination thereof.

In another aspect, the detectable label is a peptide tag. In one aspect, the peptide tag is located at or near the N-terminus of the polypeptide or polypeptide complex. In another aspect, the peptide tag is located or near at the C-terminus of the polypeptide or polypeptide complex. In another aspect, the peptide tag is an affinity tag or fusion tag.

In another aspect, the detectable label is a polyhistidine tag, polyarginine tag, glutathione-S-transferase (GST), maltose binding protein (MBP), chitin binding protein (CBP), Strep-tag, thioredoxin (TRX), poly (NANP), FLAG tag, ALFA-tag, V5-tag. Myc-tag, hemagglutinin (HA) tag, Spot tag, T7 tag, NE tag, or green fluorescence protein (GFP), or a combination thereof. In one aspect, the polyhistidine tag consists of from about 4 to about 10 histidine residues. In one aspect, the poly histidine tag consists of about 4, about 5, about 6, about 7, about 8, about 9, or about 10 histidine residues.

Additional examples of detectable labels and methods for introducing detectable labels into a polypeptide are known and include routine chemical, molecular biology and recombinant DNA techniques. Sec, e.g., Hnatowich et al., Science, 220(4597):613-615, 1983; Yao et al., Int. J. Mol. Sci., 17(2):194, 2016; Kimple et al., Curr. Protoc. Protein Sci., 73; Unit 9.9, 2013; Sambrook J. Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1):DOI: 10/1080/23312025.2019.1665406.

### Polypeptides, Polynucleotides, Vectors, Cells, and Protein Production Methods

In some aspects, a polynucleotide encoding an antigen binding polypeptide or antigen polypeptide complex (e.g., an antibody or antigen binding fragment thereof) is provided herein.

In other aspects, a polypeptide provided herein comprises one or more amino acid sequences having at least 65%, at least 70%, at least 75%, at least 80%, at least 85%, at least 90%, at least 95%, at least 99%, or 100% identity to one or more of SEQ ID NOs: 381-502, 632-633, 720-721, 724-725, 728-729, 732-733, 736-737, 740-741, 744-745, 748-749, 752-753, 760-761, 887-926, 972-977, 984-985, 989-990, 993-994, 997-998, 1001-1002, 1005-1006, 1009-1010, 1013-1014, 1017-1018, 1021-1022, 1025-1026, 1029-1030, 1033-1034, 1037-1038, and 1041-1042.

In other aspects, a polypeptide provided herein comprises one or more amino acid sequences encoded by one or more polynucleotides having at least 65%, at least 70%, at least 75%, at least 80%, at least 85%, at least 90%, at least 95%, at least 99%, or 100% identity to one or more of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044.

In some aspects, the polypeptides provided herein comprise a CL and a CH1. In some aspects, a polypeptide provided herein has an improved isoelectric point compared to a same polypeptide not comprising a CL and a CH1. It is to be understood that “a same polypeptide not comprising a CL and a CH1” refers to a polypeptide having a same amino acid sequence as the reference polypeptide exception made for the CL and the CH1 being absent from the “same polypeptide not comprising a CL and a CH1.” For example, a reference polypeptide (polypeptide R) may comprise an amino acid sequence represented by the formula VL1-CL-VL2-VH2-VH1-CH1. “a same polypeptide not comprising a CL and a CH1” (polypeptide R′) it is to be understood as comprising an amino acid sequence that is represented by the formula VL1-VL2-VH2-VH1, wherein the VL1, the VL2, the VH1, and the VH1 in the polypeptide R comprise an amino acid sequence identical to the amino acid sequence comprised in the VL1, the VL2, the VH1, and the VH1 comprised in the polypeptide R′.

Without wishing to be bound to any particular theory, it is believed that the isoelectric point (pI) corresponds to the pH at which the antibody has no electrical charge. If the pH of the surrounding environment is below the antibody's pI, then the antibody molecule carries a net positive charge. If the pH of the surrounding environment is above the antibody's pI, then the antibody molecule carries a net negative charge. The pI of an antibody can influence its pharmacokinetics properties. The surface of most cells is negatively charged, thus antibodies (or antigen binding fragments thereof) having a net positive charge may possess advantageous pharmacokinetics properties. Increased net positive charge can also result in increased blood clearance and increased tissue retention with shorter half-life, whereas increased net negative charge can results in decreased tissue uptake and longer half-life. For an antibody to be positively charged the environmental pH needs to be below the pI of the antibody, and for an antibody to be negatively charged the environmental pH needs to be above the pI of the antibody. Physiological pH is of about 7-7.5 (generally between 7.35 and 7.45).

In other aspects, provided herein is a vector comprising a polynucleotide provided herein.

In yet other aspects, provided herein is a host cell comprising a polynucleotide or vector provided herein.

As used herein, the term “host cell” can be any type of cell, e.g., a primary cell, a cell in culture, a cell from a cell line, or a cell comprised in an organism. In some aspects, the term “host cell” refers to a cell containing a foreign nucleic acid molecule (e.g., a cell transformed, transfected, or transduced with a polynucleotide (e.g., DNA or mRNA)) as well as the progeny or potential progeny of such a cell. Progeny of such a cell may not be identical to the parent cell, e.g., due to mutations or environmental influences that may occur in succeeding generations or integration of the nucleic acid molecule into the host cell genome.

Methods which are well known to those skilled in the art can be used to construct vectors encoding antigen binding polypeptides and polypeptide complexes (e.g., CDR, VH, VL, heavy chain and/or light chain coding sequences and appropriate transcriptional and translational control signals). These methods include, for example, in vitro recombinant DNA techniques, synthetic techniques, and in vivo genetic engineering (e.g., transgenesis).

A vector can be transformed, transferred, or transduced into a host cell by conventional techniques and the resulting cell can then be cultured by conventional techniques to produce an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain). Thus, provided herein are host cells containing a polynucleotide encoding an antigen binding polypeptide or polypeptide complex comprising, e.g., comprising six CDRs, VH, VL, VH and VL, heavy chain, light chain, or heavy and light chain, or a domain thereof (e.g., one or more CDRs, VH, VL, VH and VL, heavy chain, or light chain), operably linked to a promoter for expression of such sequences in the host cell.

In some aspects, vectors encoding both heavy and light chains, or a domain thereof, individually, can be co-expressed in the host cell for expression. In some aspects, a host cell contains a vector comprising a polynucleotide encoding both a heavy chain and light chain, or a domain thereof. In some aspects, a host cell contains two different vectors, a first vector comprising a polynucleotide encoding a heavy chain or a domain thereof, and a second vector comprising a polynucleotide encoding a light chain or a domain thereof. In some aspects, a first host cell comprises a first vector comprising a polynucleotide encoding a heavy chain or a domain thereof, and a second host cell comprises a second vector comprising a polynucleotide encoding a light chain or a domain thereof. In some aspects, provided herein is a population of host cells comprising such a first host cell and such a second host cell.

In some aspects, provided herein is a population of vectors comprising a first vector comprising a polynucleotide encoding a light chain or domain thereof, and a second vector comprising a polynucleotide encoding a heavy chain or domain thereof. Alternatively, a single vector can be used which encodes, and is capable of expressing, both heavy and light chain polypeptides or a domain thereof.

A variety of host-vector systems can be utilized to express the polypeptides and polypeptide complexes provided herein. Such host-vector systems represent vehicles by which the coding sequences of interest can be produced and subsequently purified, but also represent cells which can, when transformed or transfected with the appropriate nucleotide coding sequences, express a polypeptide or polypeptide complex provided herein in situ. These include but are not limited to microorganisms such as bacteria (e.g., E. coli and B. subtilis) transformed with recombinant bacteriophage DNA, plasmid DNA or cosmid DNA expression vectors containing antibody or antigen binding fragment thereof coding sequences; yeast (e.g., Saccharomyces pichia) transformed with recombinant yeast expression vectors containing antibody or antigen binding fragment thereof coding sequences; insect cell systems infected with recombinant virus expression vectors (e.g., baculovirus) containing antibody or antigen binding fragment thereof coding sequences; plant cell systems (e.g., green algae such as Chlamydomonas reinhardtii) infected with recombinant virus expression vectors (e.g., cauliflower mosaic virus. CaMV; tobacco mosaic virus, TMV) or transformed with recombinant plasmid expression vectors (e.g., Ti plasmid) containing antibody or antigen binding fragment thereof coding sequences; or mammalian cell systems (e.g., COS (e.g., COSI or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40), YB/20, and BMT10) cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing polypeptide or polypeptide complexes provided herein are CHO cells, for example CHO cells from the CHO GS System™ (Lonza). In some aspects, cells for expressing the polypeptides or polypeptide complexes provided herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells) are used for the expression of recombinant polypeptides. For example, mammalian cells such as Chinese hamster ovary (CHO) cells in conjunction with a vector such as the major intermediate early gene promoter element from human cytomegalovirus is an effective expression system for polypeptides (Foecking M K & Hofstetter H (1986) Gene 45:101-105; and Cockett M I et al., (1990) Biotechnology 8:662-667). In some aspects, the polypeptides or polypeptide complexes provided herein are produced by HEK-293T cells. In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK. Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COSI or COS), PER.C6, VERO, HsS78Bst, HEK-293T, HepG2, SP210, R1.1, B-W, L-M, BSC1, BSC40, YB/20, BMT10) and HsS78Bst cells.

Once a polypeptide or polypeptide complex provided herein has been produced by recombinant expression, it can be purified by any method known in the art for purification of a protein or immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the polypeptides or polypeptide complexes provided herein can be fused to heterologous polypeptide sequences provided herein (e.g., peptide tags) or otherwise known in the art to facilitate purification.

In some aspects, a polypeptide or polypeptide complex provided herein is isolated or purified. Generally, an isolated polypeptide or polypeptide complex is one that is substantially free of other polypeptides or polypeptide complexes with different antigenic specificities. For example, in some aspects, a preparation of a polypeptide or polypeptide complex provided herein is substantially free of cellular material and/or chemical precursors.

A vector can be transformed, transferred, or transduced into a host cell comprised in an organism by conventional techniques. The resulting host cell comprised in an organism can produce an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain).

Additionally, the resulting host cell comprised in an organism, as well as a host cell not comprised in an organism (e.g., a cell maintained in culture in vitro), can transmit copies of the nucleotide sequence comprised in the vector to its progeny. A nucleic acid sequence comprised in the vector can integrate in the genome of the host cell, replicate with the genome of the host cell, and thus be transmitted to the progeny of the host cell. A nucleic acid sequence comprised in the vector can also not integrate into the genome of the host cell, but instead can be maintained extrachromosomally in the host cell. In this case the vector may further comprise sequence elements that allow the nucleotide sequence comprised in the vector to self replicate. Self-replicated copies of the nucleotide sequence comprised in the vector can then be transmitted to the progeny of the host cell.

A vector disclosed herein can be a DNA vector, an RNA vector (e.g., mRNA), or a combination thereof, DNA and/or RNA vectors can be introduced into isolated host cells (e.g., cell in culture in vitro) or into host cells comprised in an organism.

In some aspects, the vectors provided herein are RNA vectors (e.g., mRNA vectors).

In some aspects, the present invention comprises a nucleic acid encoding an antigen binding polypeptide or antigen binding polypeptide complex for in vivo administration. In some aspects, the nucleic acid encoding an antigen binding polypeptide or antigen binding polypeptide complex is a stabilized nucleic acid selected from a stabilized mRNA.

In some aspects, the present disclosure relates to one or more mRNA that encodes an antigen binding polypeptide or antigen binding polypeptide complex (e.g., antibody or antigen binding fragment thereof). Examples of such mRNA include, but are not limited to, a sequence at least 65%, at least 70%, at least 75%, at least 80%, at least 85%, at least 90%, at least 95%, at least 99%, or 100% identical to any one of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044, wherein in any one of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044, wherein “T” is substituted with “U.”

In some aspects, a nucleic acid encoding an antigen binding polypeptide or antigen binding polypeptide complex comprises said nucleic acid or mRNA and a carrier to form a pharmaceutical composition.

The term “mRNA.” as used herein, refers to a single stranded RNA that encodes the amino acid sequence of one or more polypeptides (e.g., an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain)). The term “mRNA.” as used herein includes in vitro transcribed RNA (IVT RNA) or synthetic RNA. An mRNA molecule may also contain a 5′ untranslated region (5′-UTR), and/or a 3′ untranslated region (3′-UTR). In some aspects, the RNA is produced by in vitro transcription or chemical synthesis. In one aspect, the mRNA is produced by in vitro transcription using a DNA template where DNA refers to a nucleic acid that contains deoxyribonucleotides.

As used herein, the term “transcription” relates to a process, wherein the genetic code in a DNA sequence is transcribed into RNA. Subsequently, the RNA may be translated into peptide or protein. According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts, and/or isolated nucleotides, enzymes, co-enzymes, and co-factors in the appropriate reaction conditions (e.g., controlled temperature, controlled pH). Preferably, cloning vectors are applied for the generation of transcripts. These cloning vectors are generally designated as transcription vectors and are according to the present invention encompassed by the term “vector”. According to the present invention, the mRNA used in the present invention preferably is in vitro transcribed mRNA (IVT-mRNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid (e.g., cDNA) and introducing it into an appropriate vector for in vitro transcription. The nucleic acid cloned and inserted into the appropriate vector may be obtained by reverse transcription of RNA, directly isolated from the source organism, amplified by PCR, synthesized in vitro, or any combination thereof, or obtained and/or manipulated by any means known in the art. In certain aspects of the present disclosure, the RNA is “replicon RNA” or simply a “replicon”, in particular “self-replicating RNA” or “self-amplifying RNA”.

In some aspects, the mRNAs provided herein contain structural elements optimized for maximal efficacy of the mRNA with respect to stability and translational efficiency (5′-cap, 5′-UTR, 3′-UTR, poly(A) sequence). In some aspects, the messenger RNA (mRNA) molecule provided herein further comprises (i) at least one 5′ untranslated region (5′-UTR), and/or (ii) at least one 3′ untranslated region (3′-UTR), operably linked to the open reading frame (ORF). As used herein, the term “5′ untranslated region”, or “5′ UTR” refers to a region of a messenger RNA (mRNA) that is directly upstream from the initiation codon, and it is important for the regulation of translation of a transcript. The 5′ UTR may comprise a 5′-cap. In some aspects, the mRNA according to the present disclosure comprises a 5′-cap. In some aspects, the mRNA of the present disclosure does not have uncapped 5′-triphosphates. In some aspects, the mRNA may be modified by a 5′-cap analog. The term “5′-cap” refers to a structure found on the 5′-end of an mRNA molecule and generally consists of a guanosine nucleotide connected to the mRNA via a 5′- to 5′-triphosphate linkage. In one aspect, this guanosine is methylated at the 7-position. Providing an mRNA with a 5′-cap or 5′-cap analog may be achieved by in vitro transcription, in which the 5′-cap is co-transcriptionally expressed into the mRNA strand, or may be attached to mRNA post-transcriptionally using capping enzymes. As used herein, the term “3′ untranslated region”, or “3′-UTR”, refers to a region of messenger RNA (mRNA) that immediately follows the translation termination codon. The 3′-UTR often contains regulatory regions that post-transcriptionally influence gene expression. Regulatory regions within the 3′-untranslated region can influence polyadenylation, translation efficiency, localization, and stability of the mRNA. The 3′ UTR may comprise 3′ poly(A) tail. As used herein, the term “3′ poly(A) tail”, or “poly(A) sequence”, or “poly-A tail” refers to an uninterrupted or interrupted sequence of adenylate residues which is typically located at the 3′-end of an mRNA molecule. Poly(A) sequences are known to those of skill in the art and may follow the 3′-UTR in the mRNAs described herein. An uninterrupted poly(A) sequence is characterized by consecutive adenylate residues. In nature, an uninterrupted poly(A) sequence is typical, mRNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

In some aspects, the invention features messenger RNA (mRNA) molecule comprising an open reading frame (ORF) of (i.e., encoding) an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain), wherein the antigen binding polypeptide comprises an amino acid sequence at least 65%, at least 70%, at least 75%, at least 80%, at least 85%, at least 90%, at least 95%, at least 99%, or 100% identical to any one of SEQ ID NOs: 381-502, 632-633, 720-721, 724-725, 728-729, 732-733, 736-737, 740-741, 744-745, 748-749, 752-753, 760-761, 887-926, 972-977, 984-985, 989-990, 993-994, 997-998, 1001-1002, 1005-1006, 1009-1010, 1013-1014, 1017-1018, 1021-1022, 1025-1026, 1029-1030, 1033-1034, 1037-1038, and 1041-1042. In some aspects, the invention features messenger RNA (mRNA) molecule comprising a nucleotide sequence at least 65%, at least 70%, at least 75%, at least 80%, at least 85%, at least 90%, at least 95%, at least 99%, or 100% identical to any one of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044, wherein in any one of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044, wherein “T” is substituted with “U”.

### Pharmaceutical Compositions and Kits

In some aspects, provided herein is a pharmaceutical composition comprising an antigen binding polypeptide, antigen binding polypeptide complex (e.g., antibody or antigen binding fragment thereof), polypeptide, polynucleotide, vector, or host cell provided herein.

In one aspect, a pharmaceutical composition provided herein comprises (1) an antigen binding polypeptide, antigen binding polypeptide complex (e.g., antibody or antigen binding fragment thereof), polypeptide, polynucleotide, vector, or host cell provided herein, and (2) a pharmaceutically acceptable carrier. The term “pharmaceutically acceptable carrier” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is known in the art. Except insofar as any conventional media or agent is incompatible with the active ingredient, its use in the therapeutic formulations is contemplated. Supplementary active ingredients can also be incorporated into the pharmaceutical compositions provided herein. In addition, various excipients, such as are commonly used in the art, can be included. These and other such compounds are described in the literature, e.g., in the Merck Index. Merck & Company. Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies. In some aspects, the pharmaceutical composition is for parenteral, intravenous or subcutaneous administration.

In some aspects, a pharmaceutical composition provided herein comprises a polynucleotide or a vector disclosed herein (e.g., an mRNA disclosed herein) complexed, or packaged in a liposome, a nanoliposome, a lipid nanoparticle, a lipoplex, a micell, a nanomicell, a nanoemulsion, an oil-in-water emulsions, a PEG-conjugated lipid nanoparticle, a polymeric nanoparticle, a lipid-polymer hybrid nanoparticle, a polysaccharidic nanocarrier, an RNA-peptide nanoparticle, an RNA-peptide nanocomplex, a biomimetic nanovesicle, a lipidoid-RNA complex, a virus-like particle, dendrimer nanoparticle, a nanogel, a metallic nanoparticle, a gold nanoparticle (AuPNs), a magnetic nanoparticle, a theranostic nanoparticle, or any combination thereof.

In some aspects, a pharmaceutical composition provided herein comprises a lipid nanoparticle composition. As used herein, a “lipid nanoparticle composition.” “lipid nanoparticle formulation.” “LNP composition.” or “LNP formulation” is a composition comprising one or more lipids. The lipids in a lipid nanoparticle composition are typically sized on the order of micrometers or smaller. In some aspects, the lipids in a lipid nanoparticle composition have an average size of less than 1 micrometer.

In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid.

As used herein, a “cationic and/or ionizable lipid” is a lipid that has a positive or partial positive charge at physiological pH. Examples of a cationic and/or ionizable lipid include, but are not limited to, a lipid including a cyclic amine group, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)). (2S)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), and mixtures thereof.

As used herein, a “structural lipid” is a fat synthesized from mixtures of long-chain and medium-chain fatty acids. A structural lipid is typically a triacylglycerol restructured or modified to change the fatty acid composition and/or positional distribution. Examples of a structural lipid include, but are not limited to, cholesterol, fecosterol, sitosterol, ergosterol, campesterol, stigmasterol, brassicasterol, tomatidine, tomatine, ursolic acid, alpha-tocopherol, and mixtures thereof.

As used herein, a “PEGylated lipid” is a lipid that has been modified to include polyethylene glycol or “PEG.” Examples of a PEGylated lipid include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, PEG-c-DOMG, PEG-DMG, PEG-DLPE, PEG-DMPE, PEG-DPPC, a PEG-DSPE lipid, and mixtures thereof.

As used herein, a “phospholipid” is a class of lipids whose molecule has a hydrophilic “head” containing a phosphate group and two hydrophobic “tails” derived from fatty acids, joined by an alcohol residue (usually a glycerol molecule). Examples of a phospholipid include, but are not limited to, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, the phospholipid is DSPC. In some aspects, the phospholipid is DOPE. In some aspects, the phospholipid is DSPC and DOPE.

Exemplary lipid nanoparticle compositions and methods for making them are described, for example, herein and in U.S. Pat. Nos. 11,524,023; 11,485,972; 10,898,574; 10,703,789; 10,702,600; 10,577,403; 10,442,756; 10,266,485; 10,064,959; 9,868,692; Int'l Pub. No, WO 2019/046809; Int'l Pub. No, WO 2020/160397; U.S. Pub. No. 2020/0306191; Xu et al., Adv. Nanobio. Res. 2:2, 2022; Cullis et al., Mol. Ther. 25(7):1467-1475, 2017; Fan et al., J. Pharm. Biomed. Anal. 192:113642, 2020; Paunovska et al., Nature Rev., 23:265-280, 2022; Buck et al., ACS Nano. 13:3754-3782, 2019; Pardi et al., Nature Comm. 8:14630, 2017; and Pardi et al., J. Control Release, 217:345-351, 2015, which are incorporated by reference herein. Such methods include, but are not limited to, back translating DNA coding for an antigen binding polypeptide or antigen binding polypeptide complex provided herein (e.g., an antibody or antigen binding fragment thereof) into an in vitro transcription (IVT) plasmid according to such published methods.

Exemplary compositions and methods related to mRNA preparation and delivery are described, for example, herein and in Int'l Pub. No, WO 2018/081638, Int'l Pub. No, WO 2017/182524, U.S. Pat. No. 9,012,219, Int'l Pub. No, WO 2016/176330, U.S. Pat. No. 9,371,511, U.S. Appl. Pub. No. 2018/0028645, U.S. Appl. Pub. No. 23018/0344838, U.S. Pub. No. 2018/0265848, U.S. Appl. Pub. No. 2017/0043037, U.S. Appl. Pub. No. 2017/0327842, U.S. Pat. No. 10,006,007, U.S. Appl. Pub. No. 2013/0261172, U.S. Appl. Pub. No. 2013/0197068, U.S. Appl. Pub. No. 2015/0038558, U.S. Appl. Pub. No. 2016/0032316, U.S. Appl. Pub. No. 2013/0111615, U.S. Appl. Pub. No. 2009/0286852, Cullis et al., Mol. Ther. 25(7):1467-1475, 2017; Pardi et al., Nature Comm. 8:14630, 2017; Pardi et al., J. Control Release, 217:345-351, 2015; and Grier et al., Mol. Ther. Nucleic Acids, 5:e306 (2016), which are incorporated by reference herein. Such methods include, but are not limited to, in vitro transcription (e.g., using PCR products or a linearized plasmid); IVT vectors; and synthesizing mRNA using, e.g., HiScribe T7 High Yield RNA Synthesis Kit (New England Biolabs), incorporating, e.g., Pseudouridine-5′-Triphosphate (TriLink BioTechnologies). In some aspects, 5′ capped mRNA can also be generated during this process using, e.g., CleanCap Reagent AG (TriLink BioTechnologies) to form, e.g., 100 bp or 120 bp poly(A) tails.

In some aspects, the pharmaceutical composition comprises (1) one or more polynucleotides encoding an antigen binding polypeptide or antigen binding polypeptide complex, and (2) a carrier. In some aspects, the pharmaceutical composition comprises (1) one or more polynucleotides encoding an antigen binding polypeptide or antigen binding polypeptide complex (e.g., antibody or antigen binding fragment thereof) provided herein, and (2) a carrier selected from the group consisting of a lipid or a lipid nanoparticle. In some aspects, the one or more polynucleotides encoding an antigen binding polypeptide or antigen binding polypeptide complex is a stabilized polynucleotide resistant to degradation or decomposition in vivo. In some aspects, the stabilized polynucleotide is capped at one end (5′ cap) and has a long polyadenylated (poly A) tail at the other end to form a stabilized mRNA having 5′ and 3′ UTRs. In some aspects, the poly A tail is about 50 bp, about 100 bp, about 120 bp, or about 150 bp. In some aspects, modified nucleosides may be incorporated into the mRNA to increase translation and/or to lower potential immunogenicity.

Also provided herein is a pharmaceutical composition comprising an antigen binding polypeptide, antigen binding polypeptide complex (e.g., antibody or antigen binding fragment thereof), polypeptide, polynucleotide, vector, or host cell provided herein, and an additional pharmaceutical agent.

In some aspects, the additional pharmaceutical agent is 25-hydroxyvitamin D, an agent that potentiates vitamin D action, an anti-viral agent, an anti-malarial agent, an antibiotic, or a combination thereof.

In some aspects, the additional pharmaceutical agent is 25-hydroxyvitamin D.

In some aspects, the agent that potentiates vitamin D action is a CYP24 inhibitor, 1,25-dihydroxyvitamin D compound, or a combination thereof.

In some aspects, the anti-viral agent is an anti-retroviral agent, an antibody against SARS-CoV-2 virus or antigen binding fragment thereof, an inhibitor of reverse transcriptase, or a combination thereof.

In some aspects, the anti-viral agent is maraviroc, enfuvirtide, amantadine, lamivudine, nevirapine, efavirenz, dolutegravir, elvitegravir, raltegravir, acyclovir and any nucleoside analog of aciclovir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, sofobuvir, bebtelovimab (LY1404, LY-CoV1404, LY3853133), or a combination thereof.

In some aspects, the anti-viral agent is bebtelovimab.

In some aspects, the anti-viral agent is Retrovir®; (3′-azido-3′-deoxypyrimidine, zidovudine), 3′-azido-3′-deoxy thymidine (AZT), HMDR; (2′,3′-dideoxycytidine, zalcitabine), VidexEC® (2′,3′dideoxyinosine, didanosine), Epivir® (lamivudine), Zerit® (stavudine), Viread® (tenofovir DF), Ziagen® (abacavir), Emtriva® (emtricitabine, FTC), Rescriptor® (delavirdine), Sustiva® (efavirenz), Viramune® (nevirapine, 11-cyclopropyl-4-methyl-5,11-dihydro-6H-dipyrido[3,2-b:2′,3′-e][1,4]diazepin-6-one), trisodium phosphonoformate, ammonium-21-tungstenato-9-antimonate, 1-β-D-ribofuranoxyl-1,2,4-triazole-3-carboxamide, Aganerase® (amprenavir), Reyataz® (atazanavir), Lexiva® (fosamprenavir). Crixivan® (indinavir), Viracept® (nelfinavir), Norvir® (ritonavir), Fortovase® or Invirase® (saquinavir), lasinavir (5(S)-(tert-butoxycarbonylamino)-4(S)-hydroxy-6-phenyl-2(R) (2,3,4-trimethoxyphenylmethyl)-hexanoyl-(L)-valyl-N-(2-methoxy-ethyl)-amide), adriamycin, KVX-478, VX-478, 141W94, AG-1343, KNI-272, U-96988, BILA-2011 BS (palinavir), polymannoacetate, Fuzeon® (enfuvirtide, T-20), Epzicom® (abacavir and lamivudine), Trizivir® (abacavir, lamivudine and zidovudine). Truvada® (emtricitabine and tenofir DF). Combivir® (lamivudine and zidovudine), Kaletra® (lopinavir and ritonavir), bebtelovimab, or a combination thereof.

In some aspects, provided herein is a kit comprising an antigen binding polypeptide, antigen binding polypeptide complex (e.g., antibody or antigen binding fragment thereof), polypeptide, polynucleotide, vector, host cell, or pharmaceutical composition provided herein, or a combination thereof. Once a pharmaceutical composition has been formulated, it can be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. In some aspects, provided herein is a kit for producing a single-dose administration unit. In one aspect, the kit contains a first container having a dried antigen binding polypeptide or polypeptide complex provided herein (e.g., antibody or antigen binding fragment thereof) and a second container having an aqueous formulation. In some aspects, provided herein is a kit containing an antigen binding polypeptide or polypeptide complex provided herein (e.g., antibody or antigen binding fragment thereof) in a single and/or multi-chambered pre-filled syringe (e.g., liquid syringe and/or lyosyringe). In some aspects, the kit further contains components for intravenous or subcutaneous administration.

In some aspects, a kit provided herein comprises one or more antigen binding polypeptides or antigen binding polypeptide complexes (e.g., antibodies or antigen binding fragments thereof) provided herein (e.g., 1, 2, 3, 4, 5 or more). In some aspects, a kit provided herein comprises an antigen binding polypeptide or antigen binding polypeptide complexes (e.g., antibodies or antigen binding fragments thereof) provided herein.

In some aspects, a kit provided herein comprises one or more pharmaceutical compositions comprising an antigen binding polypeptide complex (e.g., antibody or antigen binding fragment thereof) provided herein (e.g., 1, 2, 3, 4, 5 or more). In some aspects, a kit provided herein comprises a pharmaceutical compositions comprising an antigen binding polypeptide complexes (e.g., antibody or antigen binding fragment thereof) provided herein. In some aspects, the pharmaceutical composition further comprises a pharmaceutically acceptable carrier.

In some aspects, a kit provided herein comprises (i) one or more antigen binding polypeptide complexes (e.g., antibodies or antigen binding fragments thereof) provided herein (e.g., 1, 2, 3, 4, 5 or more), and (ii) one or more additional pharmaceutical agents (e.g., 1, 2, 3, 4, 5 or more). In some aspects, a kit provided herein comprises (i) an antigen binding polypeptide complex (e.g., antibody or antigen binding fragment thereof) provided herein, and (ii) an additional pharmaceutical agent.

In some aspects, a kit provided herein comprises (i) one or more pharmaceutical compositions comprising an antigen binding polypeptide complex (e.g., antibody or antigen binding fragment thereof) provided herein (e.g., 1, 2, 3, 4, 5 or more), and (ii) one or more additional pharmaceutical agents (e.g., 1, 2, 3, 4, 5 or more). In some aspects, a kit provided herein comprises (i) a pharmaceutical composition comprising an antigen binding polypeptide complex (e.g., antibody or antigen binding fragment thereof) provided herein, and (ii) an additional pharmaceutical agent. In some aspects, the pharmaceutical composition further comprises a pharmaceutically acceptable carrier.

In some aspects, the kit further comprises instructions for use, for example, for any of the methods of use described herein (e.g., treating or preventing a SARS-CoV-2 infection).

### Methods of Use

In some aspects, provided herein are certain methods of use of an antigen binding polypeptide, antigen binding polypeptide complex (e.g., an antibody or antigen binding fragment thereof), polypeptide, polynucleotide, vector, host cell, or pharmaceutical composition provided herein, or a combination thereof.

In some aspects, provided herein is a method of preventing or treating a SARS-CoV-2 infection, comprising administering an antigen binding polypeptide, antigen binding polypeptide complex (e.g., an antibody or antigen binding fragment thereof), polypeptide, polynucleotide, vector, host cell or pharmaceutical composition provided herein, or a combination thereof.

In some aspects, provided herein is a method of preventing or treating a SARS-CoV-2 infection in a subject in need thereof, comprising administering to the subject a therapeutically effective amount of an antigen binding polypeptide, antigen binding polypeptide complex (e.g., an antibody or antigen binding fragment thereof), polypeptide, polynucleotide, vector, host cell or pharmaceutical composition provided herein, or a combination thereof.

In some aspects, provided herein is a method of preventing or treating coronavirus disease 2019 (COVID-19), comprising administering an antigen binding polypeptide, antigen binding polypeptide complex (e.g., an antibody or antigen binding fragment thereof), polypeptide, polynucleotide, vector, host cell or pharmaceutical composition, or a combination thereof.

In some aspects, provided herein is a method of preventing or treating COVID-19 in a subject in need thereof, comprising administering to the subject a therapeutically effective amount of an antigen binding polypeptide, antigen binding polypeptide complex (e.g., an antibody or antigen binding fragment thereof), polypeptide, polynucleotide, vector, host cell or pharmaceutical composition, or a combination thereof.

In some aspects, provided herein is a method of preventing immune escape of a SARS-CoV-2 variant, comprising administering an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, provided herein is a method of preventing immune escape of a SARS-CoV-2 variant in a subject in need thereof, comprising administering to the subject a therapeutically effective amount of an antigen binding polypeptide, antigen binding polypeptide complex (e.g., an antibody or antigen binding fragment thereof), polypeptide, polynucleotide, vector, host cell or pharmaceutical composition provided herein, or a combination thereof.

In some aspects, the variant is the SARS-CoV-2 virus is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an Iota variant (e.g., B.1.526), a Lambda variant (e.g., C.37), an Epsilon variant (e.g., B.1.429, B.1.427, or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, or XBB.2.3.2), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof. In some aspects, the variant is an Omicron variant. In some aspects, the Omicron variant is B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, KP.3.1.1, or a combination or variant thereof. In some aspects, the Omicron variant is BA1.1.529, BA.1, BA.2, BA.2.12.1, BA.4/5, or a combination or variant thereof.

In some aspects, provided herein is a method of increasing neutralization potency against a SARS-CoV-2 variant, comprising administering an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, provided herein is a method of increasing neutralization potency against a SARS-CoV-2 variant in a subject in need thereof, comprising administering to the subject a therapeutically effective amount of an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, the increased neutralization potency is relative to a monospecific antigen binding polypeptide complex that specifically binds to one of the same antigens as the antigen binding polypeptide complex. In some aspects, the increased neutralization potency is relative to a mixture of monospecific antigen binding polypeptide complexes that specifically binds to the same antigens as the antigen binding polypeptide complex.

In some aspects, the variant is the SARS-CoV-2 virus is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an Iota variant (e.g., B.1.526), a Lambda variant (e.g., C.37), an Epsilon variant (e.g., B.1.429, B.1.427, or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, or XBB.2.3.2), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof. In some aspects, the variant is an Omicron variant. In some aspects, the Omicron variant is B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, KP.3.1.1, or a combination or variant thereof. In some aspects, the Omicron variant is BA1.1.529, BA.1, BA.2, BA.2.12.1, BA.4/5, or a combination or variant thereof.

In some aspects, provided herein is a method of increasing neutralization potency against multiple SARS-CoV-2 variants, comprising administering an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, provided herein is a method of increasing neutralization potency against multiple SARS-CoV-2 variants in a subject in need thereof, comprising administering to the subject a therapeutically effective amount of an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, the increased neutralization potency is relative to a monospecific antigen binding polypeptide complex that specifically binds to one of the same antigens as the antigen binding polypeptide complex. In some aspects, the increased neutralization potency is relative to a mixture of monospecific antigen binding polypeptide complexes that specifically bind to the same antigens as the antigen binding polypeptide complex.

In some aspects, the variant is the SARS-CoV-2 virus is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an Iota variant (e.g., B.1.526), a Lambda variant (e.g., C.37), an Epsilon variant (e.g., B.1.429, B.1.427, or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1 EG.5.1, FL.1.5.1, HK.3, HV.1, JD.1.1, JF.1, XBB.1, XBB.1.16, XBB.1.16.6, or XBB.2.3.2), or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof. In some aspects, the variant is an Omicron variant. In some aspects, the Omicron variant is B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, KP.3.1.1, or a combination or variant thereof. In some aspects, the Omicron variant is BA1.1.529, BA.1, BA.2, BA.2.12.1, BA.4/5, or a combination or variant thereof. In some aspects, provided herein is a method of preventing or treating a viral infection (i.e., an infection caused by a virus) in a subject comprising administering to the subject an antigen binding polypeptide, antigen binding polypeptide complex (e.g., an antibody or antigen binding fragment thereof), polypeptide, polynucleotide, vector, host cell or pharmaceutical composition provided herein, or a combination thereof, wherein the virus is selected from the group consisting of influenza virus, respiratory syncytial virus (RSV), chlamydia, adenovirdiae, mastadeno virus, aviadenovirus, herpesviridae, herpes simplex virus 1, herpes simplex virus 2, herpes simplex virus 5, herpes simplex virus 6, leviviridae, levivirus, enterobacteria phase MS2, allolevirus, poxviridae, chordopoxvirinae, parapoxvirus, avipoxvirus, capripoxvirus, leporiipoxvirus, suipoxvirus, molluscipoxvirus, entomopoxvirinae, papovaviridae, polyomavirus, papillomavirus, paramyxoviridae, paramyxovirus, parainfluenza virus 1, mobillivirus, measles virus, rubulavirus, mumps virus, pneumonovirinae, pneumovirus, me tapneumo virus, avian pneumovirus, human metapneumovirus, picornaviridae, enterovirus, rhinovirus, hepatovirus, human hepatitis A virus, cardiovirus, andaptho virus, reoviridae, orthoreovirus, orbivirus, rotavirus, cypovirus, fijivirus, phytoreovirus, oryzavirus, retroviridae, mammalian type B retroviruses, mammalian type C retroviruses, avian type C retroviruses, type D retrovirus group, BLV-HTLV retroviruses, lentivirus, human immunodeficiency virus 1, human immunodeficiency virus 2, HTLV-I and -II viruses, herpes simplex E virus. Epstein Barr virus, cytomegalovirus, hepatitis virus (HCV, HAV, HBV, HDV, HEV), Toxoplasma gondii virus, Treponema pallidium virus, human T-lymphotrophic virus, encephalitis virus. West Nile virus. Dengue virus. Varicella Zoster Virus, rubcola, mumps, rubella, spumavirus, flaviviridae, hepatitis C virus, hepadnaviridae, hepatitis B virus, togaviridae, alphavirus sindbis virus, rubivirus, rubella virus, rhabdoviridae, vesiculovirus, lyssavirus, ephemerovirus, cytorhabdo virus, necleorhabdo virus, arenaviridae, arenavirus, lymphocytic choriomeningitis virus. Ippy virus, lassa virus, coronaviridae, coronavirus, and torovirus.

In these aspects, the antigen binding polypeptide or antigen binding polypeptide complex (e.g., an antibody or antigen binding fragment thereof) is to be understood to specifically bind to at least one epitope on at least one antigen of the virus causing the viral infection. Non-limiting examples of viral antigens are influenza virus neuraminidase, influenza virus hemagglutinin, human respiratory syncytial virus (RSV)-viral proteins, RSV F glycoprotein, RSV G glycoprotein, herpes simplex virus (HSV) viral proteins, herpes simplex virus glycoproteins gB, gC, gD, and gE, chlamydia MOMP and PorB antigens, core protein, matrix protein or other protein of Dengue virus, measles virus hemagglutinin, herpes simplex virus type 2 glycoprotein gB, poliovirus 1 VP1, envelope glycoproteins of HIV 1, hepatitis B surface antigen, diptheria toxin, streptococcus 24M epitope, gonococcal pilin, pseudorabies virus g50) (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein. Serpulina hydodysenteriae protective antigen, bovine viral diarrhea glycoprotein 55. Newcastle disease virus hemagglutinin-neuraminidase, swine flu hemagglutinin, swine flu neuraminidase, foot and mouth disease virus, hog colera virus, swine influenza virus. African swine fever virus, Mycoplasma liyopneutiioniae, infectious bovine rhinotracheitis virus, infectious bovine rhinotracheitis virus glycoprotein E, glycoprotein G, infectious laryngotracheitis virus, infectious laryngotracheitis virus glycoprotein G or glycoprotein I, a glycoprotein of La Crosse virus, neonatal calf diarrhoea virus. Venezuelan equine encephalomyelitis virus, punta toro virus, murine leukemia virus, mouse mammary tumor virus, hepatitis B virus core protein and hepatitis B virus surface antigen or a fragment or derivative thereof, antigen of equine influenza virus or equine herpes virus, including equine influenza virus type A/Alaska 91 neuraminidase, equine influenza virus typeA/Miami 63 neuraminidase, equine influenza virus type A/Kentucky 81 neuraminidase equine herpes virus type 1 glycoprotein B, and equine herpes virus type 1 glycoprotein D, antigen of bovine respiratory syncytial virus or bovine parainfluenza virus, bovine respiratory syncytial virus attachment protein (BRSV G), bovine respiratory syncytial virus fusion protein (BRSV F), bovine respiratory syncytial virus nucleocapsid protein (BRSVN), bovine parainfluenza virus type 3 fusion protein, bovine parainfluenza virus type 3 hemagglutinin neuraminidase, bovine E viral diarrhoea virus glycoprotein 48 and glycoprotein 53, glycoprotein E of Dengue virus, and glycoprotein E1E2 of human hepatitis C virus.

In some aspects, provided herein is a method of preventing or treating a cancer (e.g., a solid tumor or a blood cancer) in a subject comprising administering to the subject an antigen binding polypeptide, antigen binding polypeptide complex (e.g., an antibody or antigen binding fragment thereof), polypeptide, polynucleotide, vector, host cell or pharmaceutical composition provided herein.

In these aspects, the antigen binding polypeptide or antigen binding polypeptide complex (e.g., an antibody or antigen binding fragment thereof) is to be understood to specifically bind to at least one epitope on at least one antigen associated with cancer (e.g., a tumor-associated antigens (TAAs), tumor-specific antigens (TSAs), or neoantigens), or to at least one epitope on at least one antigen selected from the group consisting of, e.g., A2AR, APRIL, ATPDase, BAFF, BAFFR, BCMA, BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25 CCR3, CCR4, CD3, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL1O, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, and WUCAM.

Delivery of the antigen binding polypeptide, antigen binding polypeptide complex (e.g., antibody or antigen binding fragment thereof), polypeptide, or antibody or antigen binding fragment thereof provided herein can be by direct administration of the antigen binding polypeptide, antigen binding polypeptide complex (e.g., antibody or antigen binding fragment thereof), polypeptide, or antibody or antigen binding fragment thereof provided herein or by an indirect method that is inclusive of delivery of a modified mRNA composition that is administered to the patient in need of treatment thereof by an antigen binding polypeptide, an antigen binding polypeptide complex (e.g., antibody or antigen binding fragment thereof), a polypeptide, or an antibody or antigen binding fragment thereof provided herein translated from the modified mRNA that codes for the antigen binding polypeptide, antigen binding polypeptide complex (e.g., antibody or antigen binding fragment thereof), polypeptide, or antibody or antigen binding fragment thereof provided herein.

As used herein, the terms “prevent” or “preventing” refer to the prevention of the onset, recurrence or spread, in whole or in part, of a disease or condition provided herein, or a symptom thereof. As used herein, the terms “treat” or “treating” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treat” can also mean prolonging survival as compared to expected survival if not receiving treatment.

As used herein. “administering” is meant a method of giving a dosage of an antigen binding polypeptide or polypeptide complex (e.g., antibody or antigen binding fragment thereof) to a subject in need thereof (e.g., a patient). Administering can be by any suitable means, including parenteral, intrapulmonary or intranasal. Parenteral infusions include, for example, intramuscular, intravenous, intraarterial, intraperitoneal or subcutaneous administration. Dosing can be by any suitable route, e.g., by injection, such as intravenous or subcutaneous injection. Various dosing schedules including, but not limited to, single or multiple administrations over various time-points, bolus administration, and pulse infusion are contemplated herein.

As used herein, a “therapeutically effective amount” is an amount of an antigen binding polypeptide or antigen binding polypeptide complex (e.g., an antibody or antigen binding fragment thereof) that is sufficient to achieve the desired effect and can vary according to the nature and severity of the condition, and the potency of the polypeptide or polypeptide complex. In one aspect, an antigen binding polypeptide or polypeptide complex can be delivered by administering a polynucleotide, vector, or host cell that encodes the antigen binding polypeptide or polypeptide complex. In another aspect, an antigen binding polypeptide or polypeptide complex thereof can be delivered by administering a pharmaceutical composition containing the polypeptide or polypeptide complex. A therapeutic effect is the relief, to at least some extent, of one or more symptoms of the disease or disorder, and can include curing a disease or disorder. “Curing” means that the symptoms of active disease are eliminated. However, certain long-term or permanent effects of a disease or disorder can exist even after a cure is obtained.

As used herein, the term “subject” means a human or a non-human mammal, e.g., a dog, cat, mouse, rat, cow, sheep, pig, goat, non-human primate or bird, e.g., chicken, as well as any other vertebrate or invertebrate. In one aspect, the subject is a human. In another aspect, the subject is a veterinary animal. In one aspect, the subject is a mammal.

In some aspects, provided herein is a method of diagnosing a subject as being infected with a SARS-CoV-2 virus or suspected of being infected with a SARS-CoV-2 virus, comprising (i) contacting a sample obtained from the subject with the antigen binding polypeptide or polypeptide complex (e.g., an antibody or antigen binding fragment thereof) provided herein; (ii) detecting the presence or absence of a virus complex which contains the polypeptide or a fragment thereof, or polypeptide complex or a fragment thereof, and a SARS-CoV-2 virus, virion or fragment thereof; and (iii) diagnosing the subject as being infected with a SARS-CoV-2 virus or suspected of being infected with a SARS-CoV-2 virus when the presence of the virus complex is detected.

In some aspects, provided herein is a method of diagnosing a subject as not being infected with a SARS-CoV-2 virus or not suspected of being infected with a SARS-CoV-2 virus, comprising (i) contacting a sample obtained from the subject with the antigen binding polypeptide or polypeptide complex (e.g., an antibody or antigen binding fragment thereof) provided herein; (ii) detecting the presence or absence of a virus complex which contains the polypeptide or a fragment thereof, or polypeptide complex or a fragment thereof, and a SARS-CoV-2 virus, virion or fragment thereof; and (iii) diagnosing the subject as not being infected with a SARS-CoV-2 virus or not being suspected of being infected with a SARS-CoV-2 virus when the presence of the virus complex is not detected.

In some aspects, provided herein is a method of diagnosing a subject as having COVID-19 or suspected of having COVID-19, comprising (i) contacting a sample obtained from the subject with an antigen binding polypeptide or polypeptide complex (e.g., an antibody or antigen binding fragment thereof) provided herein; (ii) detecting the presence or absence of a virus complex which contains the polypeptide or a fragment thereof, or polypeptide complex or a fragment thereof, and a SARS-CoV-2 virus, virion or fragment thereof; and (iii) diagnosing the subject as having COVID-19 or suspected or having COVID-19 when the presence of the virus complex is detected.

In some aspects, provided herein is a method of diagnosing a subject as not having COVID-19 or not suspected of having COVID-19, comprising (i) contacting a sample obtained from the subject with an antigen binding polypeptide or polypeptide complex (e.g., an antibody or antigen binding fragment thereof) provided herein; (ii) detecting the presence or absence of a virus complex which contains the polypeptide or a fragment thereof, or polypeptide complex or a fragment thereof, and a SARS-CoV-2 virus, virion or fragment thereof; and (iii) diagnosing the subject as not having COVID-19 or not suspected of having COVID-19 when the presence of the virus complex is not detected.

The term “sample.” as used herein, refers to a composition that is obtained or derived from a subject that contains a cellular and/or other molecular entity that is to be characterized and/or identified, for example, based on physical, biochemical, chemical, and/or physiological characteristics. A sample includes, but is not limited to, nasal fluid or discharge (e.g., from a nasal swab), tissue, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, tissue culture medium, tissue extracts such as homogenized tissue, cellular extracts, and combinations thereof.

Methods for collecting, processing and storing a sample are known, and described, for example, in Vaught et al., IARC Sci. Pub., Unit 2. Chapter 3, pp. 23-42, 2011; CDC Interim Guidelines for Collecting and Handling of Clinical Specimens for COVID-19 Testing. Oct. 25, 2021; and Minghetti et al., Recommendations for collection, transport and storage of COVID-19 biological samples, ISS working group on Translational Research COVID-19. Istituto Superiore di Sanita. Apr. 15, 2020.

Methods for detecting the binding of an antigen binding polypeptide or polypeptide complex (e.g., antibody or antigen binding fragment thereof) provided herein and a SARS-CoV-2 virus, virion or fragment thereof are also known. Such methods include, but are not limited to, immunosorbent assay (ELISA), sodium dodecyl sulfate-polyacry lamide gel electrophoresis (SDS-PAGE), immunospot assay, lateral flow assay, flow cytometry, immunohistochemistry or western blot. See, e.g., Kumar et al., VirusDisease, 31:97-105, 2020; Gong et al., Front. Mol. Biosci. Jul. 23, 2021; and Espejo et al., Am. J. Clin. Pathol., 154(3)293-304, 2020.

In some aspects, the SARS-CoV-2 variant of the SARS-CoV-2 protein (e.g., SARS-CoV-2 spike protein) to which an antigen binding polypeptide or polypeptide complex provided herein specifically binds is a variant of concern (VOC). In some aspects, the SARS-CoV-2 variant of the SARS-CoV-2 protein (e.g., SARS-CoV-2 spike protein) to which an antigen binding polypeptide or polypeptide complex provided herein specifically binds is a variant under monitoring (e.g., variant with genetic changes suspected to affect virus characteristics and some indication of posing a future risk, but further studies are needed).

In some aspects, the SARS-CoV-2 of the SARS-CoV-2 protein (e.g., SARS-CoV-2 spike protein) to which an antigen binding polypeptide or polypeptide complex provided herein specifically binds is the original Wuhan strain (WA1), a D614G spike protein mutant (e.g., D614G), an Alpha variant (e.g., B.1.1.7), a Beta variant (e.g., B.1.351), a Gamma variant (e.g., P.1), a Delta variant (e.g., B.1.617.2 or AY.1), a Kappa variant (e.g., B.1.617.1), an Iota variant (e.g., B.1.526), a Lambda variant (e.g., C.37), an Epsilon variant (e.g., B.1.429, B.1.427, or CAL.20C), a Mu variant (e.g., B.1.621), a Theta variant (e.g., P.3), a Zeta variant (e.g., P.2), an Eta variant (e.g., B.1.525), a R.1 variant, a C.1.2 variant, or an Omicron variant (e.g., B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, or XBB.2.3.2) or a combination or variant thereof. In some aspects, the SARS-CoV-2 virus is JN.1.13.1, KP.2, JN.1.16, JN.1.16.1, JN.1.13, JN.1.18, KP.2.3, LB.1, KP.3, KP.3.1.1, or a combination or variant thereof. In other aspects, the SARS-CoV-2 of SARS-CoV-2 protein (e.g., the SARS-CoV-2 spike protein) to which an antigen binding polypeptide or polypeptide complex provided herein specifically binds is an Alpha (e.g., B.1.1.7). Beta (e.g., B.1.351). Gamma (e.g., P.1), Delta (e.g., B.1.617.2), or Omicron (e.g., B.1.1.529) variant, or a combination or variant thereof. In some aspects, the variant is an Omicron variant. In some aspects, the Omicron variant is B.1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, BA.2.86, EG.5, JN.1, JD.1, EG.5.1, FL.1.5.1, HK.3, HV.1, JD.1.1, JF.1, XBB.1, XBB.1.16, XBB.1.16.6, XBB.2.3.2, JN.1.13.1, KP.2, JN.1.16, JN.1.16.1, JN.1.13, JN.1.18, KP.2.3, LB.1, KP.3, KP.3.1.1, or a combination or variant thereof. In some aspects, the Omicron variant is BA1.1.529, BA.1, BA.2, BA.2.12.1, BA.4/5, or a combination or variant thereof.

In some aspects, the spike protein has an amino acid mutation of one or more of the following: F2L, L5F, L5I, V6F, L7V, POL, S12C, S13I, Q14H, C15F, N17K, L18F, T20I, T22N, T22A, T221, Q23K, P25S, A27S, A27V, T29I, F32L, R34C, H49Y, S50L, T51I, Q52L, Q52H, L54F, L54W, F55I, P57L, H69Y, S71F, G72V, T73I, G75V, T76I, F79L, D80N, D80Y, N87Y, D88N, D88E, D88Y, D88A, V90F, T95A, T95I, E96D, K97T, S98F, R102I, I105L, D111N, K113R, L118F, V130A, E132D, C136R, D138H, L141-, L141F, G142V, G142-, V143F, V143-, Y144-, Y144V, Y145H, H146Y, K147E, N148S, S151I, W152C, M153T, M153V, M153I, E154V, F157L, R158S, L176F, M177I, D178N, G181V, L189F, R190K, I203M, I210-, R214L, D215Y, D215G, L216F, Q218L, F220L, S221L, A222V, A222P, D228H, L229F, Q239R, T240I, L242F, A243S, A243V, H245R, H245Y, R246K, D253Y, D253G, S254F, S256L, W258L, G261V, G261R, A262S, Y265C, V267L, R273S, E281Q, A288S, L293V, D294E, P295S, E298G, T307I, V308L, E309Q, Q314K, Q314L, Q314H, T315I, Q321L, T323I, P330S, A344S, T345S, A348T, A348S, N354K, R357K, V367F, V382L, P384L, V395I, R403K, V407I, A411S, G413R, K417T, N439K, N440K, L441I, G446V, R457K, K458Q, G476S, S477N, P479L, V483A, E484Q, E484K, Q493L, S494P, L452R, Y453F, Y508H, N501Y, H519Q, A520S, A522V, K529E, G545S, T547I, L552F, T553I, E554D, K558N, A570V, T572I, D574Y, E583D, I584V, S596I, I598V, N603H, Q613H, D614G, V615F, T618A, P621S, V622F, V622I, V622A, A623S, H625Y, A626V, P631S, W633R, G639V, S640F, A647S, A647V, E654Z, E654K, H655Y, N658Y, A668S, A672V, Q675R, Q675H, T676S, T676I, Q677H, Q677R, T678I, P681L, P681R, P681H, R682W, A684S, A684T, V687L, A688V, A688S, S689I, S691F, S698L, N703D, S704L, V705F, A706V, I714M, T716I, I720V, T724A, M731I, T732A, T732I, G744V, D745G, N751D, L754F, R765S, R765H, T768I, G769A, A771S, T778I, Q779H, E780Q, A783S, D808V, D808G, P809S, I818V, L822F, D830H, D830Y, Q836P, Q836L, G838D, A845S, A845D, A845V, A845D, A845S, A846V, R847I, K854R, N856S, T859I, D867N, A879V, A879S, F888L, A893E, A893V, E918V, L922F, A924S, A924V, S929I, D936H, D936Y, L938F, S939F, T941I, G946V, A958S, N969S, L981F, T1006I, V1008T, T1009I, A1016S, A1020V, F1052L, P1053T, L1063F, V1065L, A1070V, Q1071H, E1072V, K1073N, A1078V, A1078S, G1085R, K1086N, R1091L, H1101Y, V1104L, P1112L, D1118Y, T1120I, V1122L, S1123P, G1124V, G1124C, V1129A, I1130M, T1136I, D1139H, L1141F, D1146H, S1147L, D1153Y, P1162Q, P1162S, P1162Q, P1162S, D1163Y, G1167V, D1168H, V1176F, N1187Y, K1191N, N1192T, E1195Q, L1203F, K1205N, E1207A, I219V, G1219S, I1221T, V1228L, M1229I, V1230L, T1231I, T1231A, C1235F, M1237I, M1237V, M1237T, T1238I, K1245N, C1247F, G1251R, D1259H, S1261F, P1263L, V1264L, a deletion of residues 69 and 70, a deletion of residues 246-252 and D253N, or a combination thereof.

In another aspect, the antigen binding polypeptides and antigen binding polypeptide complexes provided herein potently inhibit one or more SARS-CoV-2 virus variants.

In some aspects, the antigen binding polypeptides disclosed herein have a structure, from amino-terminus to carboxy-terminus, represented by:
(a)
VL1-L1-CL-L2-VL2-L3-VH2-L4-VH1-L5-CH1;
VL1-L1-CL-L2-VH2-L3-VL2-L4-VH1-L5-CH1;
VL1-L1-CH1-L2-VL2-L3-VH2-L4-VH1-L5-CL;
VL1-L1-CH1-L2-VH2-L3-VL2-L4-VH1-L5-CL;
VH1-L1-CL-L2-VL2-L3-VH2-L4-VL1-L5-CH1;
VH1-L1-CL-L2-VH2-L3-VL2-L4-VL1-L5-CH1;
VH1-L1-CH1-L2-VL2-L3-VH2-L4-VL1-L5-CL;
VH1-L1-CH1-L2-VH2-L3-VL2-L4-VL1-L5-CL;
VL2-L1-CL-L2-VL1-L3-VH1-L4-VH2-L5-CH1;
VL2-L1-CL-L2-VH1-L3-VL1-L4-VH2-L5-CH1;
VL2-L1-CH1-L2-VL1-L3-VH1-L4-VH2-L5-CL;
VL2-L1-CH1-L2-VH1-L3-VL1-L4-VH2-L5-CL;
VH2-L1-CL-L2-VL1-L3-VH1-L4-VL2-L5-CH1;
VH2-L1-CL-L2-VH1-L3-VL1-L4-VL2-L5-CH1;
VH2-L1-CH1-L2-VL1-L3-VH1-L4-VL2-L5-CL; or
VH2-L1-CH1-L2-VH1-L3-VL1-L4-VL2-L5-CL; or
(b)
VL1-CL-L1-VL2-L2-VH2-L3-VH1-CH1;
VL1-CL-L1-VH2-L2-VL2-L3-VH1-CH1;
VL1-CH1-L1-VL2-L2-VH2-L3-VH1-CL;
VL1-CH1-L1-VH2-L2-VL2-L3-VH1-CL;
VH1-CL-L1-VL2-L2-VH2-L3-VL1-CH1;
VH1-CL-L1-VH2-L2-VL2-L3-VL1-CH1;
VH1-CH1-L1-VL2-L2-VH2-L3-VL1-CL;
VH1-CH1-L1-VH2-L2-VL2-L3-VL1-CL;
VL2-CL-L1-VL1-L2-VH1-L3-VH2-CH1;
VL2-CL-L1-VH1-L2-VL1-L3-VH2-CH1;
VL2-CH1-L1-VL1-L2-VH1-L3-VH2-CL;
VL2-CH1-L1-VH1-L2-VL1-L3-VH2-CL;
VH2-CL-L1-VL1-L2-VH1-L3-VL2-CH1;
VH2-CL-L1-VH1-L2-VL1-L3-VL2-CH1;
VH2-CH1-L1-VL1-L2-VH1-L3-VL2-CL; or
VH2-CH1-L1-VH1-L2-VL1-L3-VL2-CL; or
(c)
VL1-L1-VH1-L2-VL2-L3-CL-L4-VH2-L5-CH1;
VL1-L1-VH1-L2-VL2-L3-CH1-L4-VH2-L5-CL;
VL1-L1-VH1-L2-VH2-L3-CL-L4-VL2-L5-CH1;
VL1-L1-VH1-L2-VH2-L3-CH1-L4-VL2-L5-CL;
VL1-L1-VL2-L2-VH1-L3-CL-L4-VH2-L5-CH1;
VL1-L1-VL2-L2-VH1-L3-CH1-L4-VH2-L5-CL;
VL1-L1-VH2-L2-VH1-L3-CL-L4-VL2-L5-CH1;
VL1-L1-VH2-L2-VH1-L3-CH1-L4-VL2-L5-CL;
VH1-L1-VL1-L2-VL2-L3-CL-L4-VH2-L5-CH1;
VH1-L1-VL1-L2-VL2-L3-CH1-L4-VH2-L5-CL;
VH1-L1-VL1-L2-VH2-L3-CL-L4-VL2-L5-CH1;
VH1-L1-VL1-L2-VH2-L3-CH1-L4-VL2-L5-CL;
VH1-L1-VL2-L2-VL1-L3-CL-L4-VH2-L5-CH1;
VH1-L1-VL2-L2-VL1-L3-CH1-L4-VH2-L5-CL;
VH1-L1-VH2-L2-VL1-L3-CL-L4-VL2-L5-CH1;
VH1-L1-VH2-L2-VL1-L3-CH1-L4-VL2-L5-CL;
VL2-L1-VL1-L2-VH2-L3-CL-L4-VH1-L5-CH1;
VL2-L1-VL1-L2-VH2-L3-CH1-L4-VH1-L5-CL;
VL2-L1-VH1-L2-VH2-L3-CL-L4-VL1-L5-CH1;
VL2-L1-VH1-L2-VH2-L3-CH1-L4-VL1-L5-CL;
VL2-L1-VH2-L2-VL1-L3-CL-L4-VH1-L5-CH1;
VL2-L1-VH2-L2-VL1-L3-CH1-L4-VH1-L5-CL;
VL2-L1-VH2-L2-VH1-L3-CL-L4-VL1-L5-CH1;
VL2-L1-VH2-L2-VH1-L3-CH1-L4-VL1-L5-CL;
VH2-L1-VL1-L2-VL2-L3-CL-L4-VH1-L5-CH1;
VH2-L1-VL1-L2-VL2-L3-CH1-L4-VH1-L5-CL;
VH2-L1-VH1-L2-VL2-L3-CL-L4-VL1-L5-CH1;
VH2-L1-VH1-L2-VL2-L3-CH1-L4-VL1-L5-CL;
VH2-L1-VL2-L2-VL1-L3-CL-L4-VH1-L5-CH1;
VH2-L1-VL2-L2-VL1-L3-CH1-L4-VH1-L5-CL;
VH2-L1-VL2-L2-VH1-L3-CL-L4-VL1-L5-CH1; or
VH2-L1-VL2-L2-VH1-L3-CH1-L4-VL1-L5-CL; or
(d)
VL1-L1-CL-L2-VH1-L3-CH1-L4-VL2-L5-VH2;
VL1-L1-CL-L2-VH1-L3-CH1-L4-VH2-L5-VL2;
VL1-L1-CL-L2-VL2-L3-CH1-L4-VH1-L5-VH2;
VL1-L1-CL-L2-VH2-L3-CH1-L4-VH1-L5-VL2;
VL1-L1-CH1-L2-VH1-L3-CL-L4-VL2-L5-VH2;
VL1-L1-CH1-L2-VH1-L3-CL-L4-VH2-L5-VL2;
VL1-L1-CH1-L2-VL2-L3-CL-L4-VH1-L5-VH2;
VL1-L1-CH1-L2-VH2-L3-CL-L4-VH1-L5-VL2;
VH1-L1-CL-L2-VL1-L3-CH1-L4-VL2-L5-VH2;
VH1-L1-CL-L2-VL1-L3-CH1-L4-VH2-L5-VL2;
VH1-L1-CL-L2-VL2-L3-CH1-L4-VL1-L5-VH2;
VH1-L1-CL-L2-VH2-L3-CH1-L4-VL1-L5-VL2;
VH1-L1-CH1-L2-VL1-L3-CL-L4-VL2-L5-VH2;
VH1-L1-CH1-L2-VL1-L3-CL-L4-VH2-L5-VL2;
VH1-L1-CH1-L2-VL2-L3-CL-L4-VL1-L5-VH2;
VH1-L1-CH1-L2-VH2-L3-CL-L4-VL1-L5-VL2;
VL2-L1-CL-L2-VL1-L3-CH1-L4-VH2-L5-VH1;
VL2-L1-CL-L2-VH1-L3-CH1-L4-VH2-L5-VL1;
VL2-L1-CL-L2-VH2-L3-CH1-L4-VL1-L5-VH1;
VL2-L1-CL-L2-VH2-L3-CH1-L4-VH1-L5-VL1;
VL2-L1-CH1-L2-VL1-L3-CL-L4-VH2-L5-VH1;
VL2-L1-CH1-L2-VH1-L3-CL-L4-VH2-L5-VL1;
VL2-L1-CH1-L2-VH2-L3-CL-L4-VL1-L5-VH1;
VL2-L1-CH1-L2-VH2-L3-CL-L4-VH1-L5-VL1;
VH2-L1-CL-L2-VL1-L3-CH1-L4-VL2-L5-VH1;
VH2-L1-CL-L2-VH1-L3-CH1-L4-VL2-L5-VL1;
VH2-L1-CL-L2-VL2-L3-CH1-L4-VL1-L5-VH1;
VH2-L1-CL-L2-VL2-L3-CH1-L4-VH1-L5-VL1;
VH2-L1-CH1-L2-VL1-L3-CL-L4-VL2-L5-VH1;
VH2-L1-CH1-L2-VH1-L3-CL-L4-VL2-L5-VL1;
VH2-L1-CH1-L2-VL2-L3-CL-L4-VL1-L5-VH1; or
VH2-L1-CH1-L2-VL2-L3-CL-L4-VH1-L5-VL1; or
(e)
VL1-L1-CL-L2-VH1-L3-CH1;
VL1-L1-CH1-L2-VH1-L3-CL;
VH1-L1-CL-L2-VL1-L3-CH1; or
VH1-L1-CH1-L2-VL1-L3-CL; or
(f)
VL1-L1-VH1-L2-CL; or
VH1-L1-VL1-L2-CL; or
(g)
VL1-L1-VH1-L2-CH1; or
VH1-L1-VL1-L2-CH1; or
(h)
VL1-L1-VL2-L2-CH1; or
VL2-L1-VL1-L2-CH1; or
(i)
VH1-L1-VH2-L2-CL; or
VH2-L1-VH1-L2-CL; or
(j)
VL1-L1-VL2-L2-VL3-L3-CH1;
VL1-L1-VL3-L2-VL2-L3-CH1;
VL2-L1-VL1-L2-VL3-L3-CH1;
VL2-L1-VL3-L2-VL1-L3-CH1;
VL3-L1-VL1-L2-VL2-L3-CH1; or
VL3-L1-VL2-L2-VL1-L3-CH1; or
(k)
VH1-L1-VH2-L2-VH3-L3-CL;
VH1-L1-VH3-L2-VH2-L3-CL;
VH2-L1-VH1-L2-VH3-L3-CL;
VH2-L1-VH3-L2-VH1-L3-CL;
VH3-L1-VH1-L2-VH2-L3-CL; or
VH3-L1-VH2-L2-VH1-L3-CL; or
(1)
VL1-L1-VL2-L2-VH2-L3-VH1-L4-CL-L5-CH1;
VL1-L1-VL2-L2-VH2-L3-VH1-L4-CH1-L5-CL;
VL1-L1-VH2-L2-VL2-L3-VH1-L4-CL-L5-CH1;
VL1-L1-VH2-L2-VL2-L3-VH1-L4-CH1-L5-CL;
VH1-L1-VL2-L2-VH2-L3-VL1-L4-CL-L5-CH1;
VH1-L1-VL2-L2-VH2-L3-VL1-L4-CH1-L5-CL;
VH1-L1-VH2-L2-VL2-L3-VL1-L4-CL-L5-CH1;
VH1-L1-VH2-L2-VL2-L3-VL1-L4-CH1-L5-CL;
VL2-L1-VL1-L2-VH1-L3-VH2-L4-CL-L5-CH1;
VL2-L1-VL1-L2-VH1-L3-VH2-L4-CH1-L5-CL;
VL2-L1-VH1-L2-VL1-L3-VH2-L4-CL-L5-CH1;
VL2-L1-VH1-L2-VL1-L3-VH2-L4-CH1-L5-CL;
VH2-L1-VL1-L2-VH1-L3-VL2-L4-CL-L5-CH1;
VH2-L1-VL1-L2-VH1-L3-VL2-L4-CH1-L5-CL;
VH2-L1-VH1-L2-VL1-L3-VL2-L4-CL-L5-CH1; or
VH2-L1-VH1-L2-VL1-L3-VL2-L4-CH1-L5-CL;
wherein:
   CL is an immunoglobulin light chain constant region;
   CH1 is an immunoglobulin heavy chain constant region 1; and
   Ln (e.g., L1, L2, etc.) are optional amino acid linkers;
   and
   any one or more of the VL (VL1 and/or VL2) comprises:
   (i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, and 878, 1045, 1050, 1055, 1060, and 1065;
   (ii) a LCDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and
   (iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and/or
   any one or more of the VH (VH1 and/or VH2) comprises:
   (i) a HCDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067;
   (ii) a HCDR2 having an amino acid sequence that is at least 65%, at least 70%, at least 75%, at least 80%, at least 85%, at least 90%, at least 95%, at least 99%, or 100% identical to SEQ ID NO: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and
   (iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; and/or
   any one or more of the VL (VL1 and/or VL2) comprises:
   an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and/or
   any one or more of the VH (VH1 and/or VH2) comprises:
   an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptide complexes disclosed herein comprise a first polypeptide and a second polypeptide, wherein:
(a)
the first polypeptide has a structure, from amino-terminus to carboxy-terminus, represented by:
VL1-L1-CL-L2-VL2-L3-VH2-L4-VH1-L5-CH1;
VL1-L1-CL-L2-VH2-L3-VL2-L4-VH1-L5-CH1;
VL1-L1-CH1-L2-VL2-L3-VH2-L4-VH1-L5-CL;
VL1-L1-CH1-L2-VH2-L3-VL2-L4-VH1-L5-CL;
VH1-L1-CL-L2-VL2-L3-VH2-L4-VL1-L5-CH1;
VH1-L1-CL-L2-VH2-L3-VL2-L4-VL1-L5-CH1;
VH1-L1-CH1-L2-VL2-L3-VH2-L4-VL1-L5-CL;
VH1-L1-CH1-L2-VH2-L3-VL2-L4-VL1-L5-CL;
VL2-L1-CL-L2-VL1-L3-VH1-L4-VH2-L5-CH1;
VL2-L1-CL-L2-VH1-L3-VL1-L4-VH2-L5-CH1;
VL2-L1-CH1-L2-VL1-L3-VH1-L4-VH2-L5-CL;
VL2-L1-CH1-L2-VH1-L3-VL1-L4-VH2-L5-CL;
VH2-L1-CL-L2-VL1-L3-VH1-L4-VL2-L5-CH1;
VH2-L1-CL-L2-VH1-L3-VL1-L4-VL2-L5-CH1;
VH2-L1-CH1-L2-VL1-L3-VH1-L4-VL2-L5-CL; or
VH2-L1-CH1-L2-VH1-L3-VL1-L4-VL2-L5-CL;
the second polypeptide has a structure, from amino-terminus to carboxy-terminus, represented by:
VL3-L6-CL-L7-VL4-L8-VH4-L9-VH3-L10-CH1;
VL3-L6-CL-L7-VH4-L8-VL4-L9-VH3-L10-CH1;
VL3-L6-CH1-L7-VL4-L8-VH4-L9-VH3-L10-CL;
VL3-L6-CH1-L7-VH4-L8-VL4-L9-VH3-L10-CL;
VH3-L6-CL-L7-VL4-L8-VH4-L9-VL3-L10-CH1;
VH3-L6-CL-L7-VH4-L8-VL4-L9-VL3-L10-CH1;
VH3-L6-CH1-L7-VL4-L8-VH4-L9-VL3-L10-CL;
VH3-L6-CH1-L7-VH4-L8-VL4-L9-VL3-L10-CL;
VL4-L6-CL-L7-VL3-L8-VH3-L9-VH4-L10-CH1;
VL4-L6-CL-L7-VH3-L8-VL3-L9-VH4-L10-CH1;
VL4-L6-CH1-L7-VL3-L8-VH3-L9-VH4-L10-CL;
VL4-L6-CH1-L7-VH3-L8-VL3-L9-VH4-L10-CL;
VH4-L6-CL-L7-VL3-L8-VH3-L9-VL4-L10-CH1;
VH4-L6-CL-L7-VH3-L8-VL3-L9-VL4-L10-CH1;
VH4-L6-CH1-L7-VL3-L8-VH3-L9-VL4-L10-CL; or
VH4-L6-CH1-L7-VH3-L8-VL3-L9-VL4-L10-CL; or
(b)
the first polypeptide has a structure, from amino-terminus to carboxy-terminus, represented by:
VL1-CL-L1-VL2-L2-VH2-L3-VH1-CH1;
VL1-CL-L1-VH2-L2-VL2-L3-VH1-CH1;
VL1-CH1-L1-VL2-L2-VH2-L3-VH1-CL;
VL1-CH1-L1-VH2-L2-VL2-L3-VH1-CL;
VH1-CL-L1-VL2-L2-VH2-L3-VL1-CH1;
VH1-CL-L1-VH2-L2-VL2-L3-VL1-CH1;
VH1-CH1-L1-VL2-L2-VH2-L3-VL1-CL;
VH1-CH1-L1-VH2-L2-VL2-L3-VL1-CL;
VL2-CL-L1-VL1-L2-VH1-L3-VH2-CH1;
VL2-CL-L1-VH1-L2-VL1-L3-VH2-CH1;
VL2-CH1-L1-VL1-L2-VH1-L3-VH2-CL;
VL2-CH1-L1-VH1-L2-VL1-L3-VH2-CL;
VH2-CL-L1-VL1-L2-VH1-L3-VL2-CH1;
VH2-CL-L1-VH1-L2-VL1-L3-VL2-CH1;
VH2-CH1-L1-VL1-L2-VH1-L3-VL2-CL; or
VH2-CH1-L1-VH1-L2-VL1-L3-VL2-CL;
the second polypeptide has a structure, from amino-terminus to carboxy-terminus, represented by:
VL3-CL-L4-VL4-L5-VH4-L6-VH3-CH1;
VL3-CL-L4-VH4-L5-VL4-L6-VH3-CH1;
VL3-CH1-L4-VL4-L5-VH4-L6-VH3-CL;
VL3-CH1-L4-VH4-L5-VL4-L6-VH3-CL;
VH3-CL-L4-VL4-L5-VH4-L6-VL3-CH1;
VH3-CL-L4-VH4-L5-VL4-L6-VL3-CH1;
VH3-CH1-L4-VL4-L5-VH4-L6-VL3-CL;
VH3-CH1-L4-VH4-L5-VL4-L6-VL3-CL;
VL4-CL-L4-VL3-L5-VH3-L6-VH4-CH1;
VL4-CL-L4-VH3-L5-VL3-L6-VH4-CH1;
VL4-CH1-L4-VL3-L5-VH3-L6-VH4-CL;
VL4-CH1-L4-VH3-L5-VL3-L6-VH4-CL;
VH4-CL-L4-VL3-L5-VH3-L6-VL4-CH1;
VH4-CL-L4-VH3-L5-VL3-L6-VL4-CH1;
VH4-CH1-L4-VL3-L5-VH3-L6-VL4-CL; or
VH4-CH1-L4-VH3-L5-VL3-L6-VL4-CL; or
(c)
the first polypeptide has a structure, from amino-terminus to carboxy-terminus, represented by:
VL1-L1-VH1-L2-VL2-L3-CL-L4-VH2-L5-CH1;
VL1-L1-VH1-L2-VL2-L3-CH1-L4-VH2-L5-CL;
VL1-L1-VH1-L2-VH2-L3-CL-L4-VL2-L5-CH1;
VL1-L1-VH1-L2-VH2-L3-CH1-L4-VL2-L5-CL;
VL1-L1-VL2-L2-VH1-L3-CL-L4-VH2-L5-CH1;
VL1-L1-VL2-L2-VH1-L3-CH1-L4-VH2-L5-CL;
VL1-L1-VH2-L2-VH1-L3-CL-L4-VL2-L5-CH1;
VL1-L1-VH2-L2-VH1-L3-CH1-L4-VL2-L5-CL;
VH1-L1-VL1-L2-VL2-L3-CL-L4-VH2-L5-CH1;
VH1-L1-VL1-L2-VL2-L3-CH1-L4-VH2-L5-CL;
VH1-L1-VL1-L2-VH2-L3-CL-L4-VL2-L5-CH1;
VH1-L1-VL1-L2-VH2-L3-CH1-L4-VL2-L5-CL;
VH1-L1-VL2-L2-VL1-L3-CL-L4-VH2-L5-CH1;
VH1-L1-VL2-L2-VL1-L3-CH1-L4-VH2-L5-CL;
VH1-L1-VH2-L2-VL1-L3-CL-L4-VL2-L5-CH1;
VH1-L1-VH2-L2-VL1-L3-CH1-L4-VL2-L5-CL;
VL2-L1-VL1-L2-VH2-L3-CL-L4-VH1-L5-CH1;
VL2-L1-VL1-L2-VH2-L3-CH1-L4-VH1-L5-CL;
VL2-L1-VH1-L2-VH2-L3-CL-L4-VL1-L5-CH1;
VL2-L1-VH1-L2-VH2-L3-CH1-L4-VL1-L5-CL;
VL2-L1-VH2-L2-VL1-L3-CL-L4-VH1-L5-CH1;
VL2-L1-VH2-L2-VL1-L3-CH1-L4-VH1-L5-CL;
VL2-L1-VH2-L2-VH1-L3-CL-L4-VL1-L5-CH1;
VL2-L1-VH2-L2-VH1-L3-CH1-L4-VL1-L5-CL;
VH2-L1-VL1-L2-VL2-L3-CL-L4-VH1-L5-CH1;
VH2-L1-VL1-L2-VL2-L3-CH1-L4-VH1-L5-CL;
VH2-L1-VH1-L2-VL2-L3-CL-L4-VL1-L5-CH1;
VH2-L1-VH1-L2-VL2-L3-CH1-L4-VL1-L5-CL;
VH2-L1-VL2-L2-VL1-L3-CL-L4-VH1-L5-CH1;
VH2-L1-VL2-L2-VL1-L3-CH1-L4-VH1-L5-CL;
VH2-L1-VL2-L2-VH1-L3-CL-L4-VL1-L5-CH1; or
VH2-L1-VL2-L2-VH1-L3-CH1-L4-VL1-L5-CL;
the second polypeptide has a structure, from amino-terminus to carboxy-terminus, represented by:
VL3-L6-VH3-L7-VL4-L8-CL-L9-VH4-L10-CH1;
VL3-L6-VH3-L7-VL4-L8-CH1-L9-VH4-L10-CL;
VL3-L6-VH3-L7-VH4-L8-CL-L9-VL4-L10-CH1;
VL3-L6-VH3-L7-VH4-L8-CH1-L9-VL4-L10-CL;
VL3-L6-VL4-L7-VH3-L8-CL-L9-VH4-L10-CH1;
VL3-L6-VL4-L7-VH3-L8-CH1-L9-VH4-L10-CL;
VL3-L6-VH4-L7-VH3-L8-CL-L9-VL4-L10-CH1;
VL3-L6-VH4-L7-VH3-L8-CH1-L9-VL4-L10-CL;
VH3-L6-VL3-L7-VL4-L8-CL-L9-VH4-L10-CH1;
VH3-L6-VL3-L7-VL4-L8-CH1-L9-VH4-L10-CL;
VH3-L6-VL3-L7-VH4-L8-CL-L9-VL4-L10-CH1;
VH3-L6-VL3-L7-VH4-L8-CH1-L9-VL4-L10-CL;
VH3-L6-VL4-L7-VL3-L8-CL-L9-VH4-L10-CH1;
VH3-L6-VL4-L7-VL3-L8-CH1-L9-VH4-L10-CL;
VH3-L6-VH4-L7-VL3-L8-CL-L9-VL4-L10-CH1;
VH3-L6-VH4-L7-VL3-L8-CH1-L9-VL4-L10-CL;
VL4-L6-VL3-L7-VH4-L8-CL-L9-VH3-L10-CH1;
VL4-L6-VL3-L7-VH4-L8-CH1-L9-VH3-L10-CL;
VL4-L6-VH3-L7-VH4-L8-CL-L9-VL3-L10-CH1;
VL4-L6-VH3-L7-VH4-L8-CH1-L9-VL3-L10-CL;
VL4-L6-VH4-L7-VL3-L8-CL-L9-VH3-L10-CH1;
VL4-L6-VH4-L7-VL3-L8-CH1-L9-VH3-L10-CL;
VL4-L6-VH4-L7-VH3-L8-CL-L9-VL3-L10-CH1;
VL4-L6-VH4-L7-VH3-L8-CH1-L9-VL3-L10-CL;
VH4-L6-VL3-L7-VL4-L8-CL-L9-VH3-L10-CH1;
VH4-L6-VL3-L7-VL4-L8-CH1-L9-VH3-L10-CL;
VH4-L6-VH3-L7-VL4-L8-CL-L9-VL3-L10-CH1;
VH4-L6-VH3-L7-VL4-L8-CH1-L9-VL3-L10-CL;
VH4-L6-VL4-L7-VL3-L8-CL-L9-VH3-L10-CH1;
VH4-L6-VL4-L7-VL3-L8-CH1-L9-VH3-L10-CL;
VH4-L6-VL4-L7-VH3-L8-CL-L9-VL3-L10-CH1; or
VH4-L6-VL4-L7-VH3-L8-CH1-L9-VL3-L10-CL; or
(d)
the first polypeptide has a structure, from amino-terminus to carboxy-terminus, represented by:
VL1-L1-CL-L2-VH1-L3-CH1-L4-VL2-L5-VH2;
VL1-L1-CL-L2-VH1-L3-CH1-L4-VH2-L5-VL2;
VL1-L1-CL-L2-VL2-L3-CH1-L4-VH1-L5-VH2;
VL1-L1-CL-L2-VH2-L3-CH1-L4-VH1-L5-VL2;
VL1-L1-CH1-L2-VH1-L3-CL-L4-VL2-L5-VH2;
VL1-L1-CH1-L2-VH1-L3-CL-L4-VH2-L5-VL2;
VL1-L1-CH1-L2-VL2-L3-CL-L4-VH1-L5-VH2;
VL1-L1-CH1-L2-VH2-L3-CL-L4-VH1-L5-VL2;
VH1-L1-CL-L2-VL1-L3-CH1-L4-VL2-L5-VH2;
VH1-L1-CL-L2-VL1-L3-CH1-L4-VH2-L5-VL2;
VH1-L1-CL-L2-VL2-L3-CH1-L4-VL1-L5-VH2;
VH1-L1-CL-L2-VH2-L3-CH1-L4-VL1-L5-VL2;
VH1-L1-CH1-L2-VL1-L3-CL-L4-VL2-L5-VH2;
VH1-L1-CH1-L2-VL1-L3-CL-L4-VH2-L5-VL2;
VH1-L1-CH1-L2-VL2-L3-CL-L4-VL1-L5-VH2;
VH1-L1-CH1-L2-VH2-L3-CL-L4-VL1-L5-VL2;
VL2-L1-CL-L2-VL1-L3-CH1-L4-VH2-L5-VH1;
VL2-L1-CL-L2-VH1-L3-CH1-L4-VH2-L5-VL1;
VL2-L1-CL-L2-VH2-L3-CH1-L4-VL1-L5-VH1;
VL2-L1-CL-L2-VH2-L3-CH1-L4-VH1-L5-VL1;
VL2-L1-CH1-L2-VL1-L3-CL-L4-VH2-L5-VH1;
VL2-L1-CH1-L2-VH1-L3-CL-L4-VH2-L5-VL1;
VL2-L1-CH1-L2-VH2-L3-CL-L4-VL1-L5-VH1;
VL2-L1-CH1-L2-VH2-L3-CL-L4-VH1-L5-VL1;
VH2-L1-CL-L2-VL1-L3-CH1-L4-VL2-L5-VH1;
VH2-L1-CL-L2-VH1-L3-CH1-L4-VL2-L5-VL1;
VH2-L1-CL-L2-VL2-L3-CH1-L4-VL1-L5-VH1;
VH2-L1-CL-L2-VL2-L3-CH1-L4-VH1-L5-VL1;
VH2-L1-CH1-L2-VL1-L3-CL-L4-VL2-L5-VH1;
VH2-L1-CH1-L2-VH1-L3-CL-L4-VL2-L5-VL1;
VH2-L1-CH1-L2-VL2-L3-CL-L4-VL1-L5-VH1; or
VH2-L1-CH1-L2-VL2-L3-CL-L4-VH1-L5-VL1;
the second polypeptide has a structure, from amino-terminus to carboxy-terminus, represented by:
VL3-L6-CL-L7-VH3-L8-CH1-L9-VL4-L10-VH4;
VL3-L6-CL-L7-VH3-L8-CH1-L9-VH4-L10-VL4;
VL3-L6-CL-L7-VL4-L8-CH1-L9-VH3-L10-VH4;
VL3-L6-CL-L7-VH4-L8-CH1-L9-VH3-L10-VL4;
VL3-L6-CH1-L7-VH3-L8-CL-L9-VL4-L10-VH4;
VL3-L6-CH1-L7-VH3-L8-CL-L9-VH4-L10-VL4;
VL3-L6-CH1-L7-VL4-L8-CL-L9-VH3-L10-VH4;
VL3-L6-CH1-L7-VH4-L8-CL-L9-VH3-L10-VL4;
VH3-L6-CL-L7-VL3-L8-CH1-L9-VL4-L10-VH4;
VH3-L6-CL-L7-VL3-L8-CH1-L9-VH4-L10-VL4;
VH3-L6-CL-L7-VL4-L8-CH1-L9-VL3-L10-VH4;
VH3-L6-CL-L7-VH4-L8-CH1-L9-VL3-L10-VL4;
VH3-L6-CH1-L7-VL3-L8-CL-L9-VL4-L10-VH4;
VH3-L6-CH1-L7-VL3-L8-CL-L9-VH4-L10-VL4;
VH3-L6-CH1-L7-VL4-L8-CL-L9-VL3-L10-VH4;
VH3-L6-CH1-L7-VH4-L8-CL-L9-VL3-L10-VL4;
VL4-L6-CL-L7-VL3-L8-CH1-L9-VH4-L10-VH3;
VL4-L6-CL-L7-VH3-L8-CH1-L9-VH4-L10-VL3;
VL4-L6-CL-L7-VH4-L8-CH1-L9-VL3-L10-VH3;
VL4-L6-CL-L7-VH4-L8-CH1-L9-VH3-L10-VL3;
VL4-L6-CH1-L7-VL3-L8-CL-L9-VH4-L10-VH3;
VL4-L6-CH1-L7-VH3-L8-CL-L9-VH4-L10-VL3;
VL4-L6-CH1-L7-VH4-L8-CL-L9-VL3-L10-VH3;
VL4-L6-CH1-L7-VH4-L8-CL-L9-VH3-L10-VL3;
VH4-L6-CL-L7-VL3-L8-CH1-L9-VL4-L10-VH3;
VH4-L6-CL-L7-VH3-L8-CH1-L9-VL4-L10-VL3;
VH4-L6-CL-L7-VL4-L8-CH1-L9-VL3-L10-VH3;
VH4-L6-CL-L7-VL4-L8-CH1-L9-VH3-L10-VL3;
VH4-L6-CH1-L7-VL3-L8-CL-L9-VL4-L10-VH3;
VH4-L6-CH1-L7-VH3-L8-CL-L9-VL4-L10-VL3;
VH4-L6-CH1-L7-VL4-L8-CL-L9-VL3-L10-VH3; or
VH4-L6-CH1-L7-VL4-L8-CL-L9-VH3-L10-VL3; or
(e)
the first polypeptide has a structure, from amino-terminus to carboxy-terminus, represented by:
VL1-L1-VH1-L2-VL2-L3-CL-L4-VH2-L5-CH1;
VL1-L1-VH1-L2-VL2-L3-CH1-L4-VH2-L5-CL;
VL1-L1-VH1-L2-VH2-L3-CL-L4-VL2-L5-CH1;
VL1-L1-VH1-L2-VH2-L3-CH1-L4-VL2-L5-CL;
VL1-L1-VL2-L2-VH1-L3-CL-L4-VH2-L5-CH1;
VL1-L1-VL2-L2-VH1-L3-CH1-L4-VH2-L5-CL;
VL1-L1-VH2-L2-VH1-L3-CL-L4-VL2-L5-CH1;
VL1-L1-VH2-L2-VH1-L3-CH1-L4-VL2-L5-CL;
VH1-L1-VL1-L2-VL2-L3-CL-L4-VH2-L5-CH1;
VH1-L1-VL1-L2-VL2-L3-CH1-L4-VH2-L5-CL;
VH1-L1-VL1-L2-VH2-L3-CL-L4-VL2-L5-CH1;
VH1-L1-VL1-L2-VH2-L3-CH1-L4-VL2-L5-CL;
VH1-L1-VL2-L2-VL1-L3-CL-L4-VH2-L5-CH1;
VH1-L1-VL2-L2-VL1-L3-CH1-L4-VH2-L5-CL;
VH1-L1-VH2-L2-VL1-L3-CL-L4-VL2-L5-CH1;
VH1-L1-VH2-L2-VL1-L3-CH1-L4-VL2-L5-CL;
VL2-L1-VL1-L2-VH2-L3-CL-L4-VH1-L5-CH1;
VL2-L1-VL1-L2-VH2-L3-CH1-L4-VH1-L5-CL;
VL2-L1-VH1-L2-VH2-L3-CL-L4-VL1-L5-CH1;
VL2-L1-VH1-L2-VH2-L3-CH1-L4-VL1-L5-CL;
VL2-L1-VH2-L2-VL1-L3-CL-L4-VH1-L5-CH1;
VL2-L1-VH2-L2-VL1-L3-CH1-L4-VH1-L5-CL;
VL2-L1-VH2-L2-VH1-L3-CL-L4-VL1-L5-CH1;
VL2-L1-VH2-L2-VH1-L3-CH1-L4-VL1-L5-CL;
VH2-L1-VL1-L2-VL2-L3-CL-L4-VH1-L5-CH1;
VH2-L1-VL1-L2-VL2-L3-CH1-L4-VH1-L5-CL;
VH2-L1-VH1-L2-VL2-L3-CL-L4-VL1-L5-CH1;
VH2-L1-VH1-L2-VL2-L3-CH1-L4-VL1-L5-CL;
VH2-L1-VL2-L2-VL1-L3-CL-L4-VH1-L5-CH1;
VH2-L1-VL2-L2-VL1-L3-CH1-L4-VH1-L5-CL;
VH2-L1-VL2-L2-VH1-L3-CL-L4-VL1-L5-CH1; or
VH2-L1-VL2-L2-VH1-L3-CH1-L4-VL1-L5-CL;
the second polypeptide has a structure, from amino-terminus to carboxy-terminus, represented by:
VL3-L6-CL-L7-VH3-L8-CH1-L9-VL4-L10-VH4;
VL3-L6-CL-L7-VH3-L8-CH1-L9-VH4-L10-VL4;
VL3-L6-CL-L7-VL4-L8-CH1-L9-VH3-L10-VH4;
VL3-L6-CL-L7-VH4-L8-CH1-L9-VH3-L10-VL4;
VL3-L6-CH1-L7-VH3-L8-CL-L9-VL4-L10-VH4;
VL3-L6-CH1-L7-VH3-L8-CL-L9-VH4-L10-VL4;
VL3-L6-CH1-L7-VL4-L8-CL-L9-VH3-L10-VH4;
VL3-L6-CH1-L7-VH4-L8-CL-L9-VH3-L10-VL4;
VH3-L6-CL-L7-VL3-L8-CH1-L9-VL4-L10-VH4;
VH3-L6-CL-L7-VL3-L8-CH1-L9-VH4-L10-VL4;
VH3-L6-CL-L7-VL4-L8-CH1-L9-VL3-L10-VH4;
VH3-L6-CL-L7-VH4-L8-CH1-L9-VL3-L10-VL4;
VH3-L6-CH1-L7-VL3-L8-CL-L9-VL4-L10-VH4;
VH3-L6-CH1-L7-VL3-L8-CL-L9-VH4-L10-VL4;
VH3-L6-CH1-L7-VL4-L8-CL-L9-VL3-L10-VH4;
VH3-L6-CH1-L7-VH4-L8-CL-L9-VL3-L10-VL4;
VL4-L6-CL-L7-VL3-L8-CH1-L9-VH4-L10-VH3;
VL4-L6-CL-L7-VH3-L8-CH1-L9-VH4-L10-VL3;
VL4-L6-CL-L7-VH4-L8-CH1-L9-VL3-L10-VH3;
VL4-L6-CL-L7-VH4-L8-CH1-L9-VH3-L10-VL3;
VL4-L6-CH1-L7-VL3-L8-CL-L9-VH4-L10-VH3;
VL4-L6-CH1-L7-VH3-L8-CL-L9-VH4-L10-VL3;
VL4-L6-CH1-L7-VH4-L8-CL-L9-VL3-L10-VH3;
VL4-L6-CH1-L7-VH4-L8-CL-L9-VH3-L10-VL3;
VH4-L6-CL-L7-VL3-L8-CH1-L9-VL4-L10-VH3;
VH4-L6-CL-L7-VH3-L8-CH1-L9-VL4-L10-VL3;
VH4-L6-CL-L7-VL4-L8-CH1-L9-VL3-L10-VH3;
VH4-L6-CL-L7-VL4-L8-CH1-L9-VH3-L10-VL3;
VH4-L6-CH1-L7-VL3-L8-CL-L9-VL4-L10-VH3;
VH4-L6-CH1-L7-VH3-L8-CL-L9-VL4-L10-VL3;
VH4-L6-CH1-L7-VL4-L8-CL-L9-VL3-L10-VH3; or
VH4-L6-CH1-L7-VL4-L8-CL-L9-VH3-L10-VL3; or
(f)
the first polypeptide has a structure, from amino-terminus to carboxy-terminus, represented by:
VL1-L1-CL-L2-VH1-L3-CH1-L4-VL2-L5-VH2;
VL1-L1-CL-L2-VH1-L3-CH1-L4-VH2-L5-VL2;
VL1-L1-CL-L2-VL2-L3-CH1-L4-VH1-L5-VH2;
VL1-L1-CL-L2-VH2-L3-CH1-L4-VH1-L5-VL2;
VL1-L1-CH1-L2-VH1-L3-CL-L4-VL2-L5-VH2;
VL1-L1-CH1-L2-VH1-L3-CL-L4-VH2-L5-VL2;
VL1-L1-CH1-L2-VL2-L3-CL-L4-VH1-L5-VH2;
VL1-L1-CH1-L2-VH2-L3-CL-L4-VH1-L5-VL2;
VH1-L1-CL-L2-VL1-L3-CH1-L4-VL2-L5-VH2;
VH1-L1-CL-L2-VL1-L3-CH1-L4-VH2-L5-VL2;
VH1-L1-CL-L2-VL2-L3-CH1-L4-VL1-L5-VH2;
VH1-L1-CL-L2-VH2-L3-CH1-L4-VL1-L5-VL2;
VH1-L1-CH1-L2-VL1-L3-CL-L4-VL2-L5-VH2;
VH1-L1-CH1-L2-VL1-L3-CL-L4-VH2-L5-VL2;
VH1-L1-CH1-L2-VL2-L3-CL-L4-VL1-L5-VH2;
VH1-L1-CH1-L2-VH2-L3-CL-L4-VL1-L5-VL2;
VL2-L1-CL-L2-VL1-L3-CH1-L4-VH2-L5-VH1;
VL2-L1-CL-L2-VH1-L3-CH1-L4-VH2-L5-VL1;
VL2-L1-CL-L2-VH2-L3-CH1-L4-VL1-L5-VH1;
VL2-L1-CL-L2-VH2-L3-CH1-L4-VH1-L5-VL1;
VL2-L1-CH1-L2-VL1-L3-CL-L4-VH2-L5-VH1;
VL2-L1-CH1-L2-VH1-L3-CL-L4-VH2-L5-VL1;
VL2-L1-CH1-L2-VH2-L3-CL-L4-VL1-L5-VH1;
VL2-L1-CH1-L2-VH2-L3-CL-L4-VH1-L5-VL1;
VH2-L1-CL-L2-VL1-L3-CH1-L4-VL2-L5-VH1;
VH2-L1-CL-L2-VH1-L3-CH1-L4-VL2-L5-VL1;
VH2-L1-CL-L2-VL2-L3-CH1-L4-VL1-L5-VH1;
VH2-L1-CL-L2-VL2-L3-CH1-L4-VH1-L5-VL1;
VH2-L1-CH1-L2-VL1-L3-CL-L4-VL2-L5-VH1;
VH2-L1-CH1-L2-VH1-L3-CL-L4-VL2-L5-VL1;
VH2-L1-CH1-L2-VL2-L3-CL-L4-VL1-L5-VH1; or
VH2-L1-CH1-L2-VL2-L3-CL-L4-VH1-L5-VL1;
the second polypeptide has a structure, from amino-terminus to carboxy-terminus, represented by:
VL3-L6-VH3-L7-VL4-L8-CL-L9-VH4-L10-CH1;
VL3-L6-VH3-L7-VL4-L8-CH1-L9-VH4-L10-CL;
VL3-L6-VH3-L7-VH4-L8-CL-L9-VL4-L10-CH1;
VL3-L6-VH3-L7-VH4-L8-CH1-L9-VL4-L10-CL;
VL3-L6-VL4-L7-VH3-L8-CL-L9-VH4-L10-CH1;
VL3-L6-VL4-L7-VH3-L8-CH1-L9-VH4-L10-CL;
VL3-L6-VH4-L7-VH3-L8-CL-L9-VL4-L10-CH1;
VL3-L6-VH4-L7-VH3-L8-CH1-L9-VL4-L10-CL;
VH3-L6-VL3-L7-VL4-L8-CL-L9-VH4-L10-CH1;
VH3-L6-VL3-L7-VL4-L8-CH1-L9-VH4-L10-CL;
VH3-L6-VL3-L7-VH4-L8-CL-L9-VL4-L10-CH1;
VH3-L6-VL3-L7-VH4-L8-CH1-L9-VL4-L10-CL;
VH3-L6-VL4-L7-VL3-L8-CL-L9-VH4-L10-CH1;
VH3-L6-VL4-L7-VL3-L8-CH1-L9-VH4-L10-CL;
VH3-L6-VH4-L7-VL3-L8-CL-L9-VL4-L10-CH1;
VH3-L6-VH4-L7-VL3-L8-CH1-L9-VL4-L10-CL;
VL4-L6-VL3-L7-VH4-L8-CL-L9-VH3-L10-CH1;
VL4-L6-VL3-L7-VH4-L8-CH1-L9-VH3-L10-CL;
VL4-L6-VH3-L7-VH4-L8-CL-L9-VL3-L10-CH1;
VL4-L6-VH3-L7-VH4-L8-CH1-L9-VL3-L10-CL;
VL4-L6-VH4-L7-VL3-L8-CL-L9-VH3-L10-CH1;
VL4-L6-VH4-L7-VL3-L8-CH1-L9-VH3-L10-CL;
VL4-L6-VH4-L7-VH3-L8-CL-L9-VL3-L10-CH1;
VL4-L6-VH4-L7-VH3-L8-CH1-L9-VL3-L10-CL;
VH4-L6-VL3-L7-VL4-L8-CL-L9-VH3-L10-CH1;
VH4-L6-VL3-L7-VL4-L8-CH1-L9-VH3-L10-CL;
VH4-L6-VH3-L7-VL4-L8-CL-L9-VL3-L10-CH1;
VH4-L6-VH3-L7-VL4-L8-CH1-L9-VL3-L10-CL;
VH4-L6-VL4-L7-VL3-L8-CL-L9-VH3-L10-CH1;
VH4-L6-VL4-L7-VL3-L8-CH1-L9-VH3-L10-CL;
VH4-L6-VL4-L7-VH3-L8-CL-L9-VL3-L10-CH1; or
VH4-L6-VL4-L7-VH3-L8-CH1-L9-VL3-L10-CL; or
(g)
the first polypeptide has a structure, from amino-terminus to carboxy-terminus, represented by:
VL1-L1-VL2-L2-VH2-L3-VH1-L4-CL-L5-CH1;
VL1-L1-VL2-L2-VH2-L3-VH1-L4-CH1-L5-CL;
VL1-L1-VH2-L2-VL2-L3-VH1-L4-CL-L5-CH1;
VL1-L1-VH2-L2-VL2-L3-VH1-L4-CH1-L5-CL;
VH1-L1-VL2-L2-VH2-L3-VL1-L4-CL-L5-CH1;
VH1-L1-VL2-L2-VH2-L3-VL1-L4-CH1-L5-CL;
VH1-L1-VH2-L2-VL2-L3-VL1-L4-CL-L5-CH1;
VH1-L1-VH2-L2-VL2-L3-VL1-L4-CH1-L5-CL;
VL2-L1-VL1-L2-VH1-L3-VH2-L4-CL-L5-CH1;
VL2-L1-VL1-L2-VH1-L3-VH2-L4-CH1-L5-CL;
VL2-L1-VH1-L2-VL1-L3-VH2-L4-CL-L5-CH1;
VL2-L1-VH1-L2-VL1-L3-VH2-L4-CH1-L5-CL;
VH2-L1-VL1-L2-VH1-L3-VL2-L4-CL-L5-CH1;
VH2-L1-VL1-L2-VH1-L3-VL2-L4-CH1-L5-CL;
VH2-L1-VH1-L2-VL1-L3-VL2-L4-CL-L5-CH1; or
VH2-L1-VH1-L2-VL1-L3-VL2-L4-CH1-L5-CL;
the second polypeptide has a structure, from amino-terminus to carboxy-terminus, represented by:
VL3-L6-VL4-L7-VH4-L8-VH3-L9-CL-L10-CH1;
VL3-L6-VL4-L7-VH4-L8-VH3-L9-CH1-L10-CL;
VL3-L6-VH4-L7-VL4-L8-VH3-L9-CL-L10-CH1;
VL3-L6-VH4-L7-VL4-L8-VH3-L9-CH1-L10-CL;
VH3-L6-VL4-L7-VH4-L8-VL3-L9-CL-L10-CH1;
VH3-L6-VL4-L7-VH4-L8-VL3-L9-CH1-L10-CL;
VH3-L6-VH4-L7-VL4-L8-VL3-L9-CL-L10-CH1;
VH3-L6-VH4-L7-VL4-L8-VL3-L9-CH1-L10-CL;
VL4-L6-VL3-L7-VH3-L8-VH4-L9-CL-L10-CH1;
VL4-L6-VL3-L7-VH3-L8-VH4-L9-CH1-L10-CL;
VL4-L6-VH3-L7-VL3-L8-VH4-L9-CL-L10-CH1;
VL4-L6-VH3-L7-VL3-L8-VH4-L9-CH1-L10-CL;
VH4-L6-VL3-L7-VH3-L8-VL4-L9-CL-L10-CH1;
VH4-L6-VL3-L7-VH3-L8-VL4-L9-CH1-L10-CL;
VH4-L6-VH3-L7-VL3-L8-VL4-L9-CL-L10-CH1; or
VH4-L6-VH3-L7-VL3-L8-VL4-L9-CH1-L10-CL; or
(h)
the first polypeptide has a structure, from amino-terminus to carboxy-terminus, represented by:
VL1-L1-CL-L2-VH1-L3-CH1;
VL1-L1-CH1-L2-VH1-L3-CL;
VH1-L1-CL-L2-VL1-L3-CH1; or
VH1-L1-CH1-L2-VL1-L3-CL;
the second polypeptide has a structure, from amino-terminus to carboxy-terminus, represented by:
VL2-L4-CL-L5-VL3-L6-VH3-L7-VH2-L8-CH1;
VL2-L4-CL-L5-VH3-L6-VL3-L7-VH2-L8-CH1;
VL2-L4-CH1-L5-VL3-L6-VH3-L7-VH2-L8-CL;
VL2-L4-CH1-L5-VH3-L6-VL3-L7-VH2-L8-CL;
VH2-L4-CL-L5-VL3-L6-VH3-L7-VL2-L8-CH1;
VH2-L4-CL-L5-VH3-L6-VL3-L7-VL2-L8-CH1;
VH2-L4-CH1-L5-VL3-L6-VH3-L7-VL2-L8-CL;
VH2-L4-CH1-L5-VH3-L6-VL3-L7-VL2-L8-CL;
VL3-L4-CL-L5-VL2-L6-VH2-L7-VH3-L8-CH1;
VL3-L4-CL-L5-VH2-L6-VL2-L7-VH3-L8-CH1;
VL3-L4-CH1-L5-VL2-L6-VH2-L7-VH3-L8-CL;
VL3-L4-CH1-L5-VH2-L6-VL2-L7-VH3-L8-CL;
VH3-L4-CL-L5-VL2-L6-VH2-L7-VL3-L8-CH1;
VH3-L4-CL-L5-VH2-L6-VL2-L7-VL3-L8-CH1;
VH3-L4-CH1-L5-VL2-L6-VH2-L7-VL3-L8-CL; or
VH3-L4-CH1-L5-VH2-L6-VL2-L7-VL3-L8-CL; or
(i)
the first polypeptide has a structure, from amino-terminus to carboxy-terminus, represented by:
VL1-L1-CL-L2-VH1-L3-CH1;
VL1-L1-CH1-L2-VH1-L3-CL;
VH1-L1-CL-L2-VL1-L3-CH1; or
VH1-L1-CH1-L2-VL1-L3-CL;
the second polypeptide has a structure, from amino-terminus to carboxy-terminus, represented by:
VL2-L4-VH2-L5-VL3-L6-CL-L7-VH3-L8-CH1;
VL2-L4-VH2-L5-VL3-L6-CH1-L7-VH3-L8-CL;
VL2-L4-VH2-L5-VH3-L6-CL-L7-VL3-L8-CH1;
VL2-L4-VH2-L5-VH3-L6-CH1-L7-VL3-L8-CL;
VL2-L4-VL3-L5-VH2-L6-CL-L7-VH3-L8-CH1;
VL2-L4-VL3-L5-VH2-L6-CH1-L7-VH3-L8-CL;
VL2-L4-VH3-L5-VH2-L6-CL-L7-VL3-L8-CH1;
VL2-L4-VH3-L5-VH2-L6-CH1-L7-VL3-L8-CL;
VH2-L4-VL2-L5-VL3-L6-CL-L7-VH3-L8-CH1;
VH2-L4-VL2-L5-VL3-L6-CH1-L7-VH3-L8-CL;
VH2-L4-VL2-L5-VH3-L6-CL-L7-VL3-L8-CH1;
VH2-L4-VL2-L5-VH3-L6-CH1-L7-VL3-L8-CL;
VH2-L4-VL3-L5-VL2-L6-CL-L7-VH3-L8-CH1;
VH2-L4-VL3-L5-VL2-L6-CH1-L7-VH3-L8-CL;
VH2-L4-VH3-L5-VL2-L6-CL-L7-VL3-L8-CH1;
VH2-L4-VH3-L5-VL2-L6-CH1-L7-VL3-L8-CL;
VL3-L4-VL2-L5-VH3-L6-CL-L7-VH2-L8-CH1;
VL3-L4-VL2-L5-VH3-L6-CH1-L7-VH2-L8-CL;
VL3-L4-VH2-L5-VH3-L6-CL-L7-VL2-L8-CH1;
VL3-L4-VH2-L5-VH3-L6-CH1-L7-VL2-L8-CL;
VL3-L4-VH3-L5-VL2-L6-CL-L7-VH2-L8-CH1;
VL3-L4-VH3-L5-VL2-L6-CH1-L7-VH2-L8-CL;
VL3-L4-VH3-L5-VH2-L6-CL-L7-VL2-L8-CH1;
VL3-L4-VH3-L5-VH2-L6-CH1-L7-VL2-L8-CL;
VH3-L4-VL2-L5-VL3-L6-CL-L7-VH2-L8-CH1;
VH3-L4-VL2-L5-VL3-L6-CH1-L7-VH2-L8-CL;
VH3-L4-VH2-L5-VL3-L6-CL-L7-VL2-L8-CH1;
VH3-L4-VH2-L5-VL3-L6-CH1-L7-VL2-L8-CL;
VH3-L4-VL3-L5-VL2-L6-CL-L7-VH2-L8-CH1;
VH3-L4-VL3-L5-VL2-L6-CH1-L7-VH2-L8-CL;
VH3-L4-VL3-L5-VH2-L6-CL-L7-VL2-L8-CH1; or
VH3-L4-VL3-L5-VH2-L6-CH1-L7-VL2-L8-CL; or
(j)
the first polypeptide has a structure, from amino-terminus to carboxy-terminus, represented by:
VL1-L1-CL-L2-VH1-L3-CH1;
VL1-L1-CH1-L2-VH1-L3-CL;
VH1-L1-CL-L2-VL1-L3-CH1; or
VH1-L1-CH1-L2-VL1-L3-CL;
the second polypeptide has a structure, from amino-terminus to carboxy-terminus, represented by:
VL2-L4-CL-L5-VH2-L6-CH1-L7-VL3-L8-VH3;
VL2-L4-CL-L5-VH2-L6-CH1-L7-VH3-L8-VL3;
VL2-L4-CL-L5-VL3-L6-CH1-L7-VH2-L8-VH3;
VL2-L4-CL-L5-VH3-L6-CH1-L7-VH2-L8-VL3;
VL2-L4-CH1-L5-VH2-L6-CL-L7-VL3-L8-VH3;
VL2-L4-CH1-L5-VH2-L6-CL-L7-VH3-L8-VL3;
VL2-L4-CH1-L5-VL3-L6-CL-L7-VH2-L8-VH3;
VL2-L4-CH1-L5-VH3-L6-CL-L7-VH2-L8-VL3;
VH2-L4-CL-L5-VL2-L6-CH1-L7-VL3-L8-VH3;
VH2-L4-CL-L5-VL2-L6-CH1-L7-VH3-L8-VL3;
VH2-L4-CL-L5-VL3-L6-CH1-L7-VL2-L8-VH3;
VH2-L4-CL-L5-VH3-L6-CH1-L7-VL2-L8-VL3;
VH2-L4-CH1-L5-VL2-L6-CL-L7-VL3-L8-VH3;
VH2-L4-CH1-L5-VL2-L6-CL-L7-VH3-L8-VL3;
VH2-L4-CH1-L5-VL3-L6-CL-L7-VL2-L8-VH3;
VH2-L4-CH1-L5-VH3-L6-CL-L7-VL2-L8-VL3;
VL3-L4-CL-L5-VL2-L6-CH1-L7-VH3-L8-VH2;
VL3-L4-CL-L5-VH2-L6-CH1-L7-VH3-L8-VL2;
VL3-L4-CL-L5-VH3-L6-CH1-L7-VL2-L8-VH2;
VL3-L4-CL-L5-VH3-L6-CH1-L7-VH2-L8-VL2;
VL3-L4-CH1-L5-VL2-L6-CL-L7-VH3-L8-VH2;
VL3-L4-CH1-L5-VH2-L6-CL-L7-VH3-L8-VL2;
VL3-L4-CH1-L5-VH3-L6-CL-L7-VL2-L8-VH2;
VL3-L4-CH1-L5-VH3-L6-CL-L7-VH2-L8-VL2;
VH3-L4-CL-L5-VL2-L6-CH1-L7-VL3-L8-VH2;
VH3-L4-CL-L5-VH2-L6-CH1-L7-VL3-L8-VL2;
VH3-L4-CL-L5-VL3-L6-CH1-L7-VL2-L8-VH2;
VH3-L4-CL-L5-VL3-L6-CH1-L7-VH2-L8-VL2;
VH3-L4-CH1-L5-VL2-L6-CL-L7-VL3-L8-VH2;
VH3-L4-CH1-L5-VH2-L6-CL-L7-VL3-L8-VL2;
VH3-L4-CH1-L5-VL3-L6-CL-L7-VL2-L8-VH2; or
VH3-L4-CH1-L5-VL3-L6-CL-L7-VH2-L8-VL2; or
(k)
the first polypeptide has a structure, from amino-terminus to carboxy-terminus, represented by:
VL1-L1-VH1-L2-CL; or
VH1-L3-VL1-L4-CL;
the second polypeptide has a structure, from amino-terminus to carboxy-terminus, represented by:
VL2-L1-VH2-L2-CH1; or
VH2-L3-VL2-L4-CH1; or
(1)
the first polypeptide has a structure, from amino-terminus to carboxy-terminus, represented by:
VL1-L1-VL2-L2-CH1; or
VL2-L1-VL1-L2-CH1;
the second polypeptide has a structure, from amino-terminus to carboxy-terminus, represented by:
VH1-L3-VH2-L4-CL; or
VH2-L3-VH1-L4-CL; or
(m)
the first polypeptide has a structure, from amino-terminus to carboxy-terminus, represented by:
VL1-L1-VL2-L2-VL3-L3-CH1;
VL1-L1-VL3-L2-VL2-L3-CH1;
VL2-L1-VL1-L2-VL3-L3-CH1;
VL2-L1-VL3-L2-VL1-L3-CH1;
VL3-L1-VL1-L2-VL2-L3-CH1; or
VL3-L1-VL2-L2-VL1-L3-CH1;
the second polypeptide has a structure, from amino-terminus to carboxy-terminus, represented by:
VH1-L4-VH2-L5-VH3-L6-CL;
VH1-L4-VH3-L5-VH2-L6-CL;
VH2-L4-VH1-L5-VH3-L6-CL;
VH2-L4-VH3-L5-VH1-L6-CL;
VH3-L4-VH1-L5-VH2-L6-CL; or
VH3-L4-VH2-L5-VH1-L6-CL; or
(n)
the first polypeptide has a structure represented by:
VL1-L1-VL2-L2-VH2-L3-VH1-L4-Fc;
VL1-L1-VH2-L2-VL2-L3-VH1-L4-Fc;
VH1-L1-VH2-L2-VL2-L3-VL1-L4-Fc; or
VH1-L1-VL2-L2-VH2-L3-VL1-L4-Fc;
the second polypeptide has a structure represented by:
VL3-L5-VL4-L6-VH4-L7-VH3-L8-Fc;
VL3-L5-VH4-L6-VL4-L7-VH3-L8-Fc;
VH3-L5-VH4-L6-VL4-L7-VL3-L8-Fc; or
VH3-L5-VL4-L6-VH4-L7-VL3-L8-Fc; or
(0)
the first polypeptide has a structure represented by:
VL1-L1-VL2-L2-VH2-L3-VH1-L4-Fc;
VL1-L1-VH2-L2-VL2-L3-VH1-L4-Fc;
VH1-L1-VH2-L2-VL2-37-VL1-L4-Fc; or
VH1-L1-VL2-L2-VH2-L3-VL1-L4-Fc;
the second polypeptide has a structure represented by:
VL3-L5-VL4-L6-VH4-L7-VH3-L8-Fc;
VL3-L5-VH4-L6-VL4-L7-VH3-L8-Fc;
VH3-L5-VH4-L6-VL4-L7-VL3-L8-Fc; or
VH3-L5-VL4-L6-VH4-L7-VL3-L8-Fc; or
(p)
the first polypeptide has a structure represented by:
VL1-L1-VL2-L2-VH2-L3-VH1-L4-Fc; or
VH1-L1-VH2-L2-VL2-L3-VL1-L4-Fc;
the second polypeptide has a structure represented by:
VL3-L5-VL4-L6-VH4-L7-VH3-L8-Fc; or
VH3-L5-VH4-L6-VL4-L7-VL3-L8-Fc; or
(q)
the first polypeptide has a structure represented by:
VL1-L1-VL2-L2-VH2-L3-VH1-L4-CH1-L5-CL-L6-Fc;
VL1-L1-VH2-L2-VL2-L3-VH1-L4-CH1-L5-CL-L6-Fc;
VH1-L1-VH2-L2-VL2-L3-VL1-L4-CH1-L5-CL-L6-Fc;
VH1-L1-VL2-L2-VH2-L3-VL1-L4-CH1-L5-CL-L6-Fc;
VL1-L1-VL2-L2-VH2-L3-VH1-L4-CL-L5-CH1-L6-Fc;
VL1-L1-VH2-L2-VL2-L3-VH1-L4-CL-L5-CH1-L6-Fc;
VH1-L1-VH2-L2-VL2-L3-VL1-L4-CL-L5-CH1-L6-Fc; or
VH1-L1-VL2-L2-VH2-L3-VL1-L4-CL-L5-CH1-L6-Fc;
wherein the second polypeptide has a structure represented by:
VL3-L7-VL4-L8-VH4-L9-VH3-L10-CH1-L11-CL-L12-Fc;
VL3-L7-VH4-L8-VL4-L9-VH3-L10-CH1-L11-CL-L12-Fc;
VH3-L7-VH4-L8-VL4-L9-VL3-L10-CH1-L11-CL-L12-Fc;
VH3-L7-VL4-L8-VH4-L9-VL3-L10-CH1-L11-CL-L12-Fc;
VL3-L7-VL4-L8-VH4-L9-VH3-L10-CL-L11-CH1-L12-Fc;
VL3-L7-VH4-L8-VL4-L9-VH3-L10-CL-L11-CH1-L12-Fc;
VH3-L7-VH4-L8-VL4-L9-VL3-L10-CL-L11-CH1-L12-Fc; or
VH3-L7-VL4-L8-VH4-L9-VL3-L10-CL-L11-CH1-L12-Fc;
wherein:
CL is an immunoglobulin light chain constant region;
CH1 is an immunoglobulin heavy chain constant region 1;
Fc is a region comprising an immunoglobulin heavy chain constant region 2 (CH2), an immunoglobulin heavy chain constant region 3 (CH3), and optionally, an immunoglobulin hinge;
Ln (e.g., L1, L2, etc.) are optional amino acid linkers;
and
any one or more of the VL (VL1, VL2, VL3 and/or VL4) comprises:
(i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, and 878, 1045, 1050, 1055, 1060, and 1065;
(ii) a LCDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and
(iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and/or
any one or more of the VH (VH1, VH2, VH3 and/or VH4) comprises:
(i) a HCDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067;
(ii) a HCDR2 having an amino acid sequence that is at least 65%, at least 70%, at least 75%, at least 80%, at least 85%, at least 90%, at least 95%, at least 99%, or 100% identical to SEQ ID NO: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and
(iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; and/or
any one or more of the VL (VL1, VL2, VL3 and/or VL4) comprises:
an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and/or
any one or more of the VH (VH1, VH2, VH3 and/or VH4) comprises:
an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptide complexes disclosed herein comprise a first polypeptide and a second polypeptide, wherein:
(a)
at least one antigen binding polypeptide has a structure represented by:
VL1-L1-VL2-L2-VH2-L3-VH1-L4-Fc-L5-Fc; VL1-L1-VH2-L2-VL2-L3-VH1-L4-Fc-L5-Fc; VH1-L1-VH2-L2-VL2-L3-VL1-L4-Fc-L5-Fc; or VH1-L1-VL2-L2-VH2-L3-VL1-L4-Fc-L5-Fc;
wherein:

Fc is a region comprising an immunoglobulin heavy chain constant region 2 (CH2), an immunoglobulin heavy chain constant region 3 (CH3), and optionally, an immunoglobulin hinge;
Ln (e.g., L1, L2, etc.) are optional amino acid linkers;
and
any one or more of the VL (VL1 and/or VL2) comprises:
(i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, and 878, 1045, 1050, 1055, 1060, and 1065;
(ii) a LCDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and
(iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and/or
any one or more of the VH (VH1 and/or VH2) comprises:
(i) a HCDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067;

(ii) a HCDR2 having an amino acid sequence that is at least 65%, at least 70%, at least 75%, at least 80%, at least 85%, at least 90%, at least 95%, at least 99%, or 100% identical to SEQ ID NO: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and
(iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; and/or
any one or more of the VL (VL1 and/or VL2) comprises:
an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and/or
any one or more of the VH (VH1 and/or VH2) comprises:
an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, the antigen binding polypeptide complexes disclosed herein comprise a polypeptide having a structure represented by:
(a)
VL1-L1-VL2-L2-VH2-L3-VH1-L4-Fc-L5-Fc; or
VH1-L1-VH2-L2-VL2-L3-VL1-L4-Fc-L5-Fc;
Fc is a region comprising an immunoglobulin heavy chain constant region 2 (CH2), an immunoglobulin heavy chain constant region 3 (CH3), and optionally, an immunoglobulin hinge;
Ln (e.g., L1, L2, etc.) are optional amino acid linkers;
and
any one or more of the VL (VL1 and/or VL2) comprises:
(i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, and 878, 1045, 1050, 1055, 1060, and 1065;
(ii) a LCDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and
(iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and/or
any one or more of the VH (VH1 and/or VH2) comprises:
(i) a HCDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067;
(ii) a HCDR2 having an amino acid sequence that is at least 65%, at least 70%, at least 75%, at least 80%, at least 85%, at least 90%, at least 95%, at least 99%, or 100% identical to SEQ ID NO: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and
(iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; and/or
any one or more of the VL (VL1 and/or VL2) comprises:
an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and/or
any one or more of the VH (VH1 and/or VH2) comprises:
an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

In some aspects, an anti-SARS-CoV-2 antibody or antigen binding fragment thereof disclosed herein comprises any one or more of the antigen binding polypeptides disclosed herein. In some aspects, an anti-SARS-CoV-2 antibody or antigen binding fragment thereof disclosed herein comprises any one of the antigen binding polypeptide complexes disclosed herein.

In some aspects, an antibody (e.g., anti-SARS-CoV-2 antibody) or antigen binding fragment thereof disclosed herein is selected from the group consisting of:
(a) an antigen binding polypeptide having a structure represented by:
VL1-L1-VH1 or VH1-L1-VL1; or
an antigen binding polypeptide having a structure represented by:
VL1-L1-VL2-L2-VH2-L3-VH1;
VL1-L1-VH2-L2-VL2-L3-VH1;
VH1-L1-VH2-L2-VL2-L3-VL1; or
VH1-L1-VL2-L2-VH2-L3-VL1;
(b) an antigen binding polypeptide having a structure represented by:
VL1-L1-VH1 or VH1-L1-VL1; or
an antigen binding polypeptide having a structure represented by:
VL1-L1-VL2-L2-VH2-L3-VH1;
VL1-L1-VH2-L2-VL2-L3-VH1;
VH1-L1-VH2-L2-VL2-L3-VL1; or
VH1-L1-VL2-L2-VH2-L3-VL1;
wherein:
Ln (e.g., L1, L2, etc.) are optional amino acid linkers;
and
any one or more of the VL (VL1 and/or VL2) comprises:
(i) a LCDR1 having an amino acid sequence that is at least 65%, at least 70%, at least 75%, at least 80%, at least 85%, at least 90%, at least 95%, at least 99%, or 100% identical to any one of SEQ ID NOs: 1, 9, 15, 23, 30, 41, 48, 55, 58, 65, 72, 79, 86, 93, 99, 105, 112, 119, 130, 138, 145, 152, 159, 166, 172, 179, 186, 192, 199, 205, 212, 219, 226, 232, 324, 627, 640, 650, 659, 676, 687, 695, 703, 712, 770, 778, 785, 794, 801, 807, 814, 827, 836, 849, 858, 865, and 878, 1045, 1050, 1055, 1060, and 1065;
(ii) a LCDR2 having an amino acid sequence that is at least 65%, at least 70%, at least 75%, at least 80%, at least 85%, at least 90%, at least 95%, at least 99%, or 100% identical to any one of SEQ ID NOs: 2, 31, 628, 641, 677, 1046, 1051, 1056, and 1061; or at least 65%, at least 70%, at least 75%, at least 80%, at least 85%, at least 90%, at least 95%, at least 99%, or 100% identical to any one of amino acid sequences AAS, GAS, LGS, GTN, GTN, SAS, SDS, DNT, EVT, DAS, DVT, RNS, DVS, EDS, DDS, KDS, VAS, WAS, YTS, EVS, NNN, SYN, and DAT; and
(iii) a LCDR3 having an amino acid sequence that is at least 65%, at least 70%, at least 75%, at least 80%, at least 85%, at least 90%, at least 95%, at least 99%, or 100% identical to any one of SEQ ID NOs: 3, 10, 16, 24, 32, 38, 42, 49, 59, 66, 73, 80, 87, 94, 100, 106, 113, 120, 126, 131, 139, 146, 153, 160, 167, 173, 180, 187, 193, 200, 206, 213, 220, 227, 233, 239, 242, 246, 249, 256, 283, 285, 287, 288, 290, 292, 294, 296, 301, 304, 306, 309, 311, 313, 316, 325, 326, 329, 642, 651, 660, 668, 678, 651, 696, 704, 713, 764, 766, 768, 771, 779, 786, 792, 795, 808, 815, 821, 830, 837, 843, 850, 856, 859, 866, 872, 879, 885, and 1066; and/or
any one or more of the VH (VH1 and/or VH2) comprises:
(i) a HCDR1 having an amino acid sequence that is at least 65%, at least 70%, at least 75%, at least 80%, at least 85%, at least 90%, at least 95%, at least 99%, or 100% identical to any one of SEQ ID NOs: 5, 12, 18, 26, 34, 44, 51, 61, 68, 75, 82, 89, 96, 102, 108, 115, 122, 133, 141, 148, 155, 162, 175, 182, 189, 195, 202, 208, 215, 222, 229, 235, 318, 629, 645, 654, 663, 671, 682, 690, 699, 707, 716, 773, 781, 788, 797, 803, 810, 817, 823, 832, 839, 845, 852, 861, 868, 874, 881, 1047, 1052, 1057, 1062, and 1067;
(ii) a HCDR2 having an amino acid sequence that is at least 65%, at least 70%, at least 75%, at least 80%, at least 85%, at least 90%, at least 95%, at least 99%, or 100% identical to SEQ ID NO: 6, 19, 27, 35, 45, 52, 56, 62, 69, 76, 83, 90, 109, 116, 123, 134, 142, 149, 156, 163, 169, 176, 183, 196, 209, 216, 223, 236, 273, 319, 328, 630, 646, 655, 664, 672, 683, 691, 708, 776, 782, 789, 798, 804, 811, 818, 824, 833, 840, 846, 853, 862, 869, 875, 882, 1048, 1053, 1058, 1063, and 1068; and
(iii) a HCDR3 having an amino acid sequence that is at least 65%, at least 70%, at least 75%, at least 80%, at least 85%, at least 90%, at least 95%, at least 99%, or 100% identical to any one of SEQ ID NOs: 7, 13, 20, 28, 36, 40, 46, 53, 63, 70, 77, 84, 91, 97, 103, 110, 117, 124, 128, 135, 143, 150, 157, 164, 170, 177, 184, 190, 197, 203, 210, 217, 224, 230, 237, 241, 244, 248, 253, 257, 259, 261, 263, 265, 267, 269, 271, 274, 276, 278, 281, 297, 320, 322, 327, 330, 332, 631, 647, 656, 665, 673, 684, 692, 700, 709, 717, 765, 767, 769, 774, 783, 790, 793, 799, 805, 812, 819, 825, 834, 841, 847, 854, 857, 863, 870, 876, 883, 886, 1049, 1054, 1059, 1064, and 1069; and/or
any one or more of the VL (VL1 and/or VL2) comprises:
an amino acid sequence that is at least 65%, at least 70%, at least 75%, at least 80%, at least 85%, at least 90%, at least 95%, at least 99%, or 100% identical to any one of SEQ ID NOs: 4, 11, 17, 25, 33, 39, 43, 50, 60, 67, 74, 81, 88, 95, 101, 107, 114, 121, 127, 132, 140, 147, 154, 161, 168, 174, 181, 188, 194, 201, 207, 214, 221, 228, 234, 240, 243, 247, 250, 251, 252, 255, 284, 286, 289, 291, 293, 295, 299, 300, 302, 303, 305, 307, 308, 310, 312, 314, 315, 317, 323, 639, 649, 658, 667, 675, 686, 694, 702, 711, 772, 780, 787, 796, 802, 809, 816, 822, 831, 838, 844, 851, 860, 867, 873, 880, and 988; and/or
any one or more of the VH (VH1 and/or VH2) comprises:
an amino acid sequence that is at least 65%, at least 70%, at least 75%, at least 80%, at least 85%, at least 90%, at least 95%, at least 99%, or 100% identical to any one of SEQ ID NOs: 8, 14, 21, 22, 29, 37, 47, 54, 57, 64, 71, 78, 85, 92, 98, 104, 111, 118, 125, 129, 136, 137, 144, 151, 158, 165, 171, 178, 185, 191, 198, 204, 211, 218, 225, 231, 238, 245, 254, 258, 260, 262, 264, 266, 268, 270, 272, 275, 277, 279, 280, 282, 298, 321, 331, 626, 644, 653, 662, 670, 681, 689, 698, 706, 715, 775, 777, 784, 791, 800, 806, 813, 820, 826, 835, 842, 848, 855, 864, 871, 877, and 884.

The following sequences are included as part of the present disclosure.

### Antigen Binding Polypeptides and Polypeptide Complexes-2

Provided herein are antigen binding polypeptides and antigen binding polypeptide complexes having certain structural features.

In some aspects, the antigen binding polypeptides provided herein comprise one or more immunoglobulin light chain variable regions (VL) and/or one or more immunoglobulin heavy chain variable regions (VH). In some aspects, the one or more VL (herein also referred to as, e.g., VL1, VL2, VL3, or VL4) specifically binds to an antigen, including an infectious disease antigen (including a viral antigen that is not a sarbecovirus antigen, a bacterial antigen, and a parasite antigen), a tumor-associated antigen, or an antigen associated with a pathology other than an infectious disease and a cancer/tumor. In some aspects, the one or more VL specifically binds to an infectious disease antigen. In some aspects, the one or more VL specifically binds to a viral antigen that is not a sarbecovirus antigen. In some aspects, the one or more VL specifically binds to a bacterial antigen. In some aspects, the one or more VL specifically binds to a parasite antigen. In some aspects, the one or more VL specifically binds to a tumor-associated antigen. In some aspects, the one or more VL specifically binds to an antigen associated with a pathology other than an infectious disease and a cancer/tumor. In some aspects, the one or more VH (herein also referred to as, e.g., VH1, VH2, VH3, or VH4) specifically binds to an antigen, including an infectious disease antigen (including a viral antigen that is not a sarbecovirus antigen, a bacterial antigen, and a parasite antigen), a tumor-associated antigen, or an antigen associated with a pathology other than an infectious disease and a cancer/tumor. In some aspects, the one or more VH specifically binds to an infectious disease antigen. In some aspects, the one or more VH specifically binds to a viral antigen that is not a sarbecovirus antigen. In some aspects, the one or more VH specifically binds to a bacterial antigen. In some aspects, the one or more VH specifically binds to a parasite antigen. In some aspects, the one or more VH specifically binds to a tumor-associated antigen. In some aspects, the one or more VH specifically binds to an antigen associated with a pathology other than an infectious disease and a cancer/tumor.

In some aspects, the antigen binding polypeptides provided herein comprise two VLs, each of the two VLs comprises a CDR1, a CDR2, and a CDR3 that are the same. In some aspects, each of the two VLs comprises a CDR1, a CDR2, and a CDR3 that are different between the two VLs.

In some aspects, the antigen binding polypeptides provided herein comprise more than two VLs. In some aspects, each of the more than two VLs comprises a CDR1, a CDR2, and a CDR3 that are the same. In some aspects, each of the more than two VLs comprises a CDR1, a CDR2, and a CDR3 that are different. In some aspects, some of the more than two VLs comprise a CDR1, a CDR2, and a CDR3 that are the same, and some of the more than two VLs comprise a CDR1, a CDR2, and a CDR3 that are different.

In some aspects, the antigen binding polypeptides provided herein comprise two VHs, each of the two VHs comprises a CDR1, a CDR2, and a CDR3 that are the same. In some aspects, each of the two VHs comprises a CDR1, a CDR2, and a CDR3 that are different between the two VHs.

In some aspects, the antigen binding polypeptides provided herein comprise more than two VHs. In some aspects, each of the more than two VHs comprises a CDR1, a CDR2, and a CDR3 that are the same. In some aspects, each of the more than two VHs comprises a CDR1, a CDR2, and a CDR3 that are different. In some aspects, some of the more than two VHs comprises a CDR1, a CDR2, and a CDR3 that are the same, and some of the more than two VHs comprises a CDR1, a CDR2, and a CDR3 that are different.

In some aspects, the antigen binding polypeptides provided herein comprise two VLs. In some aspects, the two VLs are the same. In some aspects, the two VLs are different.

In some aspects, the antigen binding polypeptides provided herein comprise more than two VLs. In some aspects, each of the more than two VLs is the same. In some aspects, each of the more than two VLs is different. In some aspects, some of the more than two VLs are the same, and some of the more than two VLs are different.

In some aspects, the antigen binding polypeptides provided herein comprise two VHs. In some aspects, the two VHs are the same. In some aspects, the two VHs are different.

In some aspects, the antigen binding polypeptides provided herein comprise more than two VHs. In some aspects, each of the more than two VHs is the same. In some aspects, each of the more than two VHs is different. In some aspects, some of the more than two VHs are the same, and some of the more than two VHs are different.

In some aspects, the antigen binding polypeptides provided herein comprise an immunoglobulin heavy chain constant region 1 (CH1). In some aspects, the antigen binding polypeptides provided herein comprise an immunoglobulin light chain constant region or domain (CL).

In some aspects, the antigen binding polypeptides provided herein comprise one or more optional amino acid linkers.

In some aspects, the antigen binding polypeptides provided herein have a structure, from amino-terminus to carboxy-terminus, represented by:
VL1-L1-CL-L2-VH1-L3-CH1;
VL1-L1-CH1-L2-VH1-L3-CL;
VH1-L1-CL-L2-VL1-L3-CH1; or
VH1-L1-CH1-L2-VL1-L3-CL;
wherein:
VL1 is an immunoglobulin light chain variable region that specifically binds to (i) a tumor-associated antigen (TAA), (ii) an infectious disease antigen that is not a sarbecovirus antigen, or (iii) an other-pathology antigen;
VH1 is an immunoglobulin heavy chain variable region that specifically binds to (i) a tumor-associated antigen (TAA), (ii) an infectious disease antigen that is not a sarbecovirus antigen, or (iii) an other-pathology antigen;
CL is an immunoglobulin light chain constant region;
CH1 is an immunoglobulin heavy chain constant region 1; and
L1-L3 are optional amino acid linkers.

In some aspects, the antigen binding polypeptides provided herein have a structure, from amino-terminus to carboxy-terminus, represented by
VL1-L1-VH1-L2-CL; or
VH1-L1-VL1-L2-CL;
wherein:
VL1 is an immunoglobulin light chain variable region that specifically binds to (i) a tumor-associated antigen (TAA), (ii) an infectious disease antigen that is not a sarbecovirus antigen, or (iii) an other-pathology antigen;
VH1 is an immunoglobulin heavy chain variable region that specifically binds to (i) a tumor-associated antigen (TAA), (ii) an infectious disease antigen that is not a sarbecovirus antigen, or (iii) an other-pathology antigen;
CL is an immunoglobulin light chain constant region; and
L1-L2 are optional amino acid linkers.

In some aspects, the antigen binding polypeptides provided herein have a structure, from amino-terminus to carboxy-terminus, represented by
VL1-L1-VH1-L2-CH1; or
VH1-L1-VL1-L2-CH1;
wherein:
VL1 is an immunoglobulin light chain variable region that specifically binds to (i) a tumor-associated antigen (TAA), (ii) an infectious disease antigen that is not a sarbecovirus antigen, or (iii) an other-pathology antigen;
VH1 is an immunoglobulin heavy chain variable region that specifically binds to (i) a tumor-associated antigen (TAA), (ii) an infectious disease antigen that is not a sarbecovirus antigen, or (iii) an other-pathology antigen;
CH1 is an immunoglobulin heavy chain constant region 1; and
L1-L2 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgen receptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD3ε, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70)-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL, Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLc, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEMI/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA. Vstm3, WT1, WUCAM, and XAGE1.

In some aspects, the antigen binding polypeptides provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, secukinumab, vunakizumab, afascvikumab, afascvikumab, bimckizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, ctrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afclimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxctan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, cmactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising a VL of an antibody selected from the group consisting of losatuxizumab vedotin, utomilumab, urclumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxctan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, secukinumab, vunakizumab, afasevikumab, afasevikumab, bimckizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, ctrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein. F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae. Streptococcus pyogenes, Streptococcus agalactiae. Streptococcus mutans, Streptococcus viridans, Streptococcus anginosus, Streptococcus intermedius. Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin). Corynebacterium ulcerans. Corynebacterium pseudotuberculosis). Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae. Mycoplasma pneumonia) antigen, a Staphylococcus (e.g., Staphylococcus aureus, coagulase-negative staphylococci (CoNS). Staphylococcus aureus alpha toxin, Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising a VL of an antibody selected from the group consisting of libivirumab, rmab, rivabazumab pegol, sctoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, cfungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor, Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanczumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides disclosed herein, comprise one or more CL region, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides disclosed herein, comprise one or more CH1 region, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional amino acid linkers. The one or more optional amino acid linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VL1-L1-CL-L2-VL2-L3-VH2-L4-VH1-L5-CH1;
VL1-L1-CL-L2-VH2-L3-VL2-L4-VH1-L5-CH1;
VL1-L1-CH1-L2-VL2-L3-VH2-L4-VH1-L5-CL;
VL1-L1-CH1-L2-VH2-L3-VL2-L4-VH1-L5-CL;
VH1-L1-CL-L2-VL2-L3-VH2-L4-VL1-L5-CH1;
VH1-L1-CL-L2-VH2-L3-VL2-L4-VL1-L5-CH1;
VH1-L1-CH1-L2-VL2-L3-VH2-L4-VL1-L5-CL;
VH1-L1-CH1-L2-VH2-L3-VL2-L4-VL1-L5-CL;
VL2-L1-CL-L2-VL1-L3-VH1-L4-VH2-L5-CH1;
VL2-L1-CL-L2-VH1-L3-VL1-L4-VH2-L5-CH1;
VL2-L1-CH1-L2-VL1-L3-VH1-L4-VH2-L5-CL;
VL2-L1-CH1-L2-VH1-L3-VL1-L4-VH2-L5-CL;
VH2-L1-CL-L2-VL1-L3-VH1-L4-VL2-L5-CH1;
VH2-L1-CL-L2-VH1-L3-VL1-L4-VL2-L5-CH1;
VH2-L1-CH1-L2-VL1-L3-VH1-L4-VL2-L5-CL; or
VH2-L1-CH1-L2-VH1-L3-VL1-L4-VL2-L5-CL;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
CL is an immunoglobulin light chain constant region;
CH1 is an immunoglobulin heavy chain constant region 1; and
L1-L5 are optional amino acid linkers.

In some aspects, the antigen binding polypeptides provided herein have a structure, from amino-terminus to carboxy-terminus, represented by:
VL1-CL-L1-VL2-L2-VH2-L3-VH1-CH1;
VL1-CL-L1-VH2-L2-VL2-L3-VH1-CH1;
VL1-CH1-L1-VL2-L2-VH2-L3-VH1-CL;
VL1-CH1-L1-VH2-L2-VL2-L3-VH1-CL;
VH1-CL-L1-VL2-L2-VH2-L3-VL1-CH1;
VH1-CL-L1-VH2-L2-VL2-L3-VL1-CH1;
VH1-CH1-L1-VL2-L2-VH2-L3-VL1-CL;
VH1-CH1-L1-VH2-L2-VL2-L3-VL1-CL;
VL2-CL-L1-VL1-L2-VH1-L3-VH2-CH1;
VL2-CL-L1-VH1-L2-VL1-L3-VH2-CH1;
VL2-CH1-L1-VL1-L2-VH1-L3-VH2-CL;
VL2-CH1-L1-VH1-L2-VL1-L3-VH2-CL;
VH2-CL-L1-VL1-L2-VH1-L3-VL2-CH1;
VH2-CL-L1-VH1-L2-VL1-L3-VL2-CH1;
VH2-CH1-L1-VL1-L2-VH1-L3-VL2-CL; or
VH2-CH1-L1-VH1-L2-VL1-L3-VL2-CL;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
CL is an immunoglobulin light chain constant region;
CH1 is an immunoglobulin heavy chain constant region 1; and
L1-L3 are amino acid linkers.

In some aspects, the antigen binding polypeptides provided herein have a structure, from amino-terminus to carboxy-terminus, represented by:
VL1-L1-VH1-L2-VL2-L3-CL-L4-VH2-L5-CH1;
VL1-L1-VH1-L2-VL2-L3-CH1-L4-VH2-L5-CL;
VL1-L1-VH1-L2-VH2-L3-CL-L4-VL2-L5-CH1;
VL1-L1-VH1-L2-VH2-L3-CH1-L4-VL2-L5-CL;
VL1-L1-VL2-L2-VH1-L3-CL-L4-VH2-L5-CH1;
VL1-L1-VL2-L2-VH1-L3-CH1-L4-VH2-L5-CL;
VL1-L1-VH2-L2-VH1-L3-CL-L4-VL2-L5-CH1;
VL1-L1-VH2-L2-VH1-L3-CH1-L4-VL2-L5-CL;
VH1-L1-VL1-L2-VL2-L3-CL-L4-VH2-L5-CH1;
VH1-L1-VL1-L2-VL2-L3-CH1-L4-VH2-L5-CL;
VH1-L1-VL1-L2-VH2-L3-CL-L4-VL2-L5-CH1;
VH1-L1-VL1-L2-VH2-L3-CH1-L4-VL2-L5-CL;
VH1-L1-VL2-L2-VL1-L3-CL-L4-VH2-L5-CH1;
VH1-L1-VL2-L2-VL1-L3-CH1-L4-VH2-L5-CL;
VH1-L1-VH2-L2-VL1-L3-CL-L4-VL2-L5-CH1;
VH1-L1-VH2-L2-VL1-L3-CH1-L4-VL2-L5-CL;
VL2-L1-VL1-L2-VH2-L3-CL-L4-VH1-L5-CH1;
VL2-L1-VL1-L2-VH2-L3-CH1-L4-VH1-L5-CL;
VL2-L1-VH1-L2-VH2-L3-CL-L4-VL1-L5-CH1;
VL2-L1-VH1-L2-VH2-L3-CH1-L4-VL1-L5-CL;
VL2-L1-VH2-L2-VL1-L3-CL-L4-VH1-L5-CH1;
VL2-L1-VH2-L2-VL1-L3-CH1-L4-VH1-L5-CL;
VL2-L1-VH2-L2-VH1-L3-CL-L4-VL1-L5-CH1;
VL2-L1-VH2-L2-VH1-L3-CH1-L4-VL1-L5-CL;
VH2-L1-VL1-L2-VL2-L3-CL-L4-VH1-L5-CH1;
VH2-L1-VL1-L2-VL2-L3-CH1-L4-VH1-L5-CL;
VH2-L1-VH1-L2-VL2-L3-CL-L4-VL1-L5-CH1;
VH2-L1-VH1-L2-VL2-L3-CH1-L4-VL1-L5-CL;
VH2-L1-VL2-L2-VL1-L3-CL-L4-VH1-L5-CH1;
VH2-L1-VL2-L2-VL1-L3-CH1-L4-VH1-L5-CL;
VH2-L1-VL2-L2-VH1-L3-CL-L4-VL1-L5-CH1; or
VH2-L1-VL2-L2-VH1-L3-CH1-L4-VL1-L5-CL;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
CL is an immunoglobulin light chain constant region;
CH1 is an immunoglobulin heavy chain constant region 1; and
L1-L5 are optional amino acid linkers.

In some aspects, the antigen binding polypeptides provided herein have a structure, from amino-terminus to carboxy-terminus, represented by:

VL1-L1-CL-L2-VH1-L3-CH1-L4-VL2-L5-VH2;
VL1-L1-CL-L2-VH1-L3-CH1-L4-VH2-L5-VL2;
VL1-L1-CL-L2-VL2-L3-CH1-L4-VH1-L5-VH2;
VL1-L1-CL-L2-VH2-L3-CH1-L4-VH1-L5-VL2;
VL1-L1-CH1-L2-VH1-L3-CL-L4-VL2-L5-VH2;
VL1-L1-CH1-L2-VH1-L3-CL-L4-VH2-L5-VL2;
VL1-L1-CH1-L2-VL2-L3-CL-L4-VH1-L5-VH2;
VL1-L1-CH1-L2-VH2-L3-CL-L4-VH1-L5-VL2;
VH1-L1-CL-L2-VL1-L3-CH1-L4-VL2-L5-VH2;
VH1-L1-CL-L2-VL1-L3-CH1-L4-VH2-L5-VL2;
VH1-L1-CL-L2-VL2-L3-CH1-L4-VL1-L5-VH2;
VH1-L1-CL-L2-VH2-L3-CH1-L4-VL1-L5-VL2;
VH1-L1-CH1-L2-VL1-L3-CL-L4-VL2-L5-VH2;
VH1-L1-CH1-L2-VL1-L3-CL-L4-VH2-L5-VL2;
VH1-L1-CH1-L2-VL2-L3-CL-L4-VL1-L5-VH2;
VH1-L1-CH1-L2-VH2-L3-CL-L4-VL1-L5-VL2;
VL2-L1-CL-L2-VL1-L3-CH1-L4-VH2-L5-VH1;
VL2-L1-CL-L2-VH1-L3-CH1-L4-VH2-L5-VL1;
VL2-L1-CL-L2-VH2-L3-CH1-L4-VL1-L5-VH1;
VL2-L1-CL-L2-VH2-L3-CH1-L4-VH1-L5-VL1;
VL2-L1-CH1-L2-VL1-L3-CL-L4-VH2-L5-VH1;
VL2-L1-CH1-L2-VH1-L3-CL-L4-VH2-L5-VL1;
VL2-L1-CH1-L2-VH2-L3-CL-L4-VL1-L5-VH1;
VL2-L1-CH1-L2-VH2-L3-CL-L4-VH1-L5-VL1;
VH2-L1-CL-L2-VL1-L3-CH1-L4-VL2-L5-VH1;
VH2-L1-CL-L2-VH1-L3-CH1-L4-VL2-L5-VL1;
VH2-L1-CL-L2-VL2-L3-CH1-L4-VL1-L5-VH1;
VH2-L1-CL-L2-VL2-L3-CH1-L4-VH1-L5-VL1;
VH2-L1-CH1-L2-VL1-L3-CL-L4-VL2-L5-VH1;
VH2-L1-CH1-L2-VH1-L3-CL-L4-VL2-L5-VL1;
VH2-L1-CH1-L2-VL2-L3-CL-L4-VL1-L5-VH1; or
VH2-L1-CH1-L2-VL2-L3-CL-L4-VH1-L5-VL1;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
CL is an immunoglobulin light chain constant region;
CH1 is an immunoglobulin heavy chain constant region 1; and
L1-L5 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgen receptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL, Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEMI/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptides provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxctan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansinc, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, secukinumab, vunakizumab, afasevikumab, afasevikumab, bimckizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, blesclumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adccatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, ctrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulcsomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afclimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, sccukinumab, vunakizumab, afasevikumab, afasevikumab, bimckizumab, bimckizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxctan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afclimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a VH of an antibody selected from the group consisting of losatuxizumab vedotin, utomilumab, urclumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansinc, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratclimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein. F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g, p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum. Plasmodium vivax. Plasmodium malariae, Plasmodium ovale, Plasmodium knowlesi) antigen, a Streptococcus (e.g., Streptococcus pneumoniae, Streptococcus pyogenes, Streptococcus agalactiae. Streptococcus mutans. Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin). Corynebacterium ulcerans, Corynebacterium pseudotuberculosis). Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS). Staphylococcus aureus alpha toxin. Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a VL of an antibody selected from the group consisting of libivirumab, rmab, rivabazumab pegol, sctoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, cdobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a VH of an antibody selected from the group consisting of libivirumab, rmab, rivabazumab pegol, sctoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor, Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P. Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A. Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanczumab, stamulumab, suptavumab, sutimlimab, tanezumab, crovalimab, and lokivetmab.

In some aspects, the antigen binding polypeptides provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VL1-L1-VL2-L2-CH1; or
VL2-L1-VL1-L2-CH1;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
CH1 is an immunoglobulin heavy chain constant region 1; and
L1-L2 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgen receptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9), CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1. MelanA/MART1, mesothelin, MHC classII, MIF, ML-IAP, MS4A1, MST1R (RON), MUC1, MUC-1, MUC16, MUC-16, MUC5AC, muthsp70)-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLc, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEMI/CD248, TEM7R, tenascin C, TGFBETA, TGF-β1, TGF-β2, TGS5, the extracellular portion of the APRIL protein. Tie2, TIGIT, TIM3, TLR, TLR2, TLR4, TLR5, TLR9, TMEF1. TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptides provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxctan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxctan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, sclicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, blesclumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adccatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, ctrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulcsomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afclimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a VL of an antibody selected from the group consisting of losatuxizumab vedotin, utomilumab, urclumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, sccukinumab, vunakizumab, afasevikumab, afasevikumab, bimckizumab, bimckizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxctan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein. F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae. Streptococcus pyogenes, Streptococcus agalactiae. Streptococcus mutans, Streptococcus viridans, Streptococcus anginosus, Streptococcus intermedius. Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin). Corynebacterium ulcerans, Corynebacterium pseudotuberculosis). Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium. Mycoplasma hyopneumoniae, Mycoplasma pneumonia) antigen, a Staphylococcus (e.g., Staphylococcus aureus, coagulase-negative staphylococci (CoNS). Staphylococcus aureus alpha toxin. Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising a VL of an antibody selected from the group consisting of libivirumab, rmab, rivabazumab pegol, sctoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a VL of an antibody selected from the group consisting of libivirumab, rmab, rivabazumab pegol, sctoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the other-pathology antigen is selected from the group consisting of Amyloid beta, β-amyloid, PCSK9, IFN-α/β receptor, Rhesus factor, nerve growth factor (NGF), DPP4, fibrin II beta chain, ACVR2B, scrum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P. Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A. Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenczumab, crizanlizumab, crotedumab, dezamizumab, domagrozumab, donanemab, eldelumab, clezanumab, emapalumab, emicizumab, eptinezumab, erenumab, evinacumab, faralimomab, fasinumab, fontolizumab, fremanezumab, frunevetmab, fulranumab, galcanczumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanczumab, stamulumab, suptavumab, sutimlimab, tanezumab, crovalimab, and lokivetmab.

In some aspects, the antigen binding polypeptides provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VH1-L1-VH2-L2-CL; or
VH2-L1-VH1-L2-CL;
wherein:
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen
CL is an immunoglobulin light chain constant region; and
L1-L2 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgen receptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxylesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1. MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEMI/CD248, TEM7R, tenascin C, TGFBETA, TGF-β1, TGF-β2, TGS5, the extracellular portion of the APRIL protein. Tie2, TIGIT, TIM3, TLR, TLR2, TLR4, TLR5, TLR9, TMEF1. TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA. Vstm3, WT1, WUCAM, and XAGE1.

In some aspects, the antigen binding polypeptides provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxctan, tetulomab, daratumumab, isatuximab, foralumab, mosunctuzumab, mosunetuzumab, cedelizumab, ibalizumab, keliximab, priliximab, tregalizumab, zanolimumab, blesclumab, iscalimab, lucatumumab, ravagalimab, sclicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansinc, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxctan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, sclicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, secukinumab, vunakizumab, afasevikumab, afascvikumab, bimekizumab, bimckizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, ctrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin modotuximab, carotuximab, adecatumumab, adccatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, sccukinumab, vunakizumab, afasevikumab, afasevikumab, bimekizumab, bimckizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, clsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, ctrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, cnoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansinc, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, sclicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, ctrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein. F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50) (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae. Streptococcus pyogenes, Streptococcus agalactiae. Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin). Corynebacterium ulcerans, Corynebacterium pseudotuberculosis). Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae. Mycoplasma pneumonia) antigen, a Staphylococcus (e.g., Staphylococcus aureus, coagulase-negative staphylococci (CoNS). Staphylococcus aureus alpha toxin. Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9), IFN-α/β receptor, Rhesus factor, nerve growth factor (NGF), DPP4, fibrin II beta chain, ACVR2B, serum amyloid A protein SOST, FGF 23, VWF, tissue factor pathway inhibitor (TFPI), 1-40) and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40) and 1-42, tau protein, dabigatran, cardiac myosin, selectin P. Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A. Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects, Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VL1-L1-VL2-L2-VL3-L3-CH1;
VL1-L1-VL3-L2-VL2-L3-CH1;
VL2-L1-VL1-L2-VL3-L3-CH1;
VL2-L1-VL3-L2-VL1-L3-CH1;
VL3-L1-VL1-L2-VL2-L3-CH1; or
VL3-L1-VL2-L2-VL1-L3-CH1;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin heavy chain variable region that specifically binds to (i) a tumor-associated antigen (TAA), (ii) an infectious disease antigen that is not a sarbecovirus antigen, or (iii) an other-pathology antigen;
VL3 is a third immunoglobulin heavy chain variable region that specifically binds to (i) a tumor-associated antigen (TAA), (ii) an infectious disease antigen that is not a sarbecovirus antigen, or (iii) an other-pathology antigen;
CH1 is an immunoglobulin heavy chain constant region 1; and
L1-L3 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgen receptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH. Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1. MelanA/MART1, mesothelin, MHC classII, MIF, ML-IAP, MS4A1, MST1R (RON), MUC1, MUC-1, MUC16, MUC-16, MUC5AC, muthsp70)-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEMI/CD248, TEM7R, tenascin C, TGFBETA, TGF-β1, TGF-β2, TGS5, the extracellular portion of the APRIL protein. Tie2, TIGIT, TIM3, TLR, TLR2, TLR4, TLR5, TLR9), TMEF1. TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptides provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, cnoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, secukinumab, vunakizumab, afasevikumab, afasevikumab, bimckizumab, bimckizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afclimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL3 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansinc, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixckizumab, netakimab, perakizumab, secukinumab, vunakizumab, afasevikumab, afasevikumab, bimckizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afclimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxctan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, cmactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofctumomab merpentan, samalizumab, imvotamab, odronextamab, plamotamab, bectumomab, inotuzumab ozogamicin, moxctumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, blesclumab, iscalimab, lucatumumab, ravagalimab, selicrelumab, tencliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL3 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9), anti-N1, anti-N2, anti-N9, anti-H1N1, anti-H3N2, anti-H5N1, anti-H7N9), anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50) (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae. Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans. Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL3 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL3 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, scrum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL3 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL3 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VH1-L1-VH2-L2-VH3-L3-CL;
VH1-L1-VH3-L2-VH2-L3-CL;
VH2-L1-VH1-L2-VH3-L3-CL;
VH2-L1-VH3-L2-VH1-L3-CL;
VH3-L1-VH1-L2-VH2-L3-CL; or
VH3-L1-VH2-L2-VH1-L3-CL;
wherein:
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
VH3 is a third immunoglobulin heavy chain variable region that specifically binds to (i) a tumor-associated antigen (TAA), (ii) an infectious disease antigen that is not a sarbecovirus antigen, or (iii) an other-pathology antigen;
CL is an immunoglobulin light chain constant region; and
L1-L3 are optional amino acid linkers.

In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90 granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40, OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLc, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptides provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, cpitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexclizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH3 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, cnoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezckimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, cnoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH3 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptides provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae. Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH3 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH3 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptides provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, clezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH3 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, devamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH3 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VL1-L1-VL2-L2-VH2-L3-VH1-L4-Fc;
VL1-L1-VH2-L2-VL2-L3-VH1-L4-Fc;
VH1-L1-VH2-L2-VL2-L3-VL1-L4-Fc; or
VH1-L1-VL2-L2-VH2-L3-VL1-L4-Fc;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
L1-L4 are optional amino acid linkers; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxylesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptides provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratclimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, aveclumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratclimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, eecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulcsomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratclimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae. Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans. Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenczumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VL1-L1-VL2-L2-VH2-L3-VH1-L4-CH1-L5-CL;
VL1-L1-VH2-L2-VL2-L3-VH1-L4-CH1-L5-CL;
VH1-L1-VH2-L2-VL2-L3-VL1-L4-CH1-L5-CL;
VH1-L1-VL2-L2-VH2-L3-VL1-L4-CH1-L5-CL;
VL1-L1-VL2-L2-VH2-L3-VH1-L4-CL-L5-CH1;
VL1-L1-VH2-L2-VL2-L3-VH1-L4-CL-L5-CH1;
VH1-L1-VH2-L2-VL2-L3-VL1-L4-CL-L5-CH1; or
VH1-L1-VL2-L2-VH2-L3-VL1-L4-CL-L5-CH1;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
L1-L5 are optional amino acid linkers;
CL is an immunoglobulin light chain constant region; and
CH1 is an immunoglobulin heavy chain constant region 1.

In some aspects, the antigen binding polypeptides provided herein have a structure, from amino-terminus to carboxy-terminus, represented by:
VL1-L1-VL2-L2-VH2-L3-VH1-L4-CH1-L5-CL-L6-Fc;
VL1-L1-VH2-L2-VL2-L3-VH1-L4-CH1-L5-CL-L6-Fc;
VH1-L1-VH2-L2-VL2-L3-VL1-L4-CH1-L5-CL-L6-Fc;
VH1-L1-VL2-L2-VH2-L3-VL1-L4-CH1-L5-CL-L6-Fc;
VL1-L1-VL2-L2-VH2-L3-VH1-L4-CL-L5-CH1-L6-Fc;
VL1-L1-VH2-L2-VL2-L3-VH1-L4-CL-L5-CH1-L6-Fc;
VH1-L1-VH2-L2-VL2-L3-VL1-L4-CL-L5-CH1-L6-Fc; or
VH1-L1-VL2-L2-VH2-L3-VL1-L4-CL-L5-CH1-L6-Fc;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
L1-L6 are optional amino acid linkers;
CL is an immunoglobulin light chain constant region;
CH1 is an immunoglobulin heavy chain constant region 1; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90 granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40, OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLc, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptides provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, cnoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezckimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, cpitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, crtumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunctuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50) (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides disclosed herein, comprise one or more CL, as indicated in the formulas representing the antigen binding polypeptides disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides disclosed herein, comprise one or more CH1, as indicated in the formulas representing the antigen binding polypeptides disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380).

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VL1-L1-VL2-L2-VH2-L3-VH1-L4-Fc-L5-Fc;
VL1-L1-VH2-L2-VL2-L3-VH1-L4-Fc-L5-Fc;
VH1-L1-VH2-L2-VL2-L3-VL1-L4-Fc-L5-Fc; or
VH1-L1-VL2-L2-VH2-L3-VL1-L4-Fc-L5-Fc;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
L1-L5 are optional amino acid linkers; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, ILSR, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70)-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLc, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptides provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulcsomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans. Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides provided herein have a structure, from amino-terminus to carboxy-terminus, represented by:
VL1-L1-VL2-L2-VH2-L3-VH1;
VL1-L1-VH2-L2-VL2-L3-VH1;
VH1-L1-VH2-L2-VL2-L3-VL1; or
VH1-L1-VL2-L2-VH2-L3-VL1;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; and
L1-L3 are optional amino acid linkers.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptides provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunctuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, crtumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansinc, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulcsomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratclimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g, p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans. Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL2 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenczumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenczumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanczumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptides provided herein have a structure, from amino-terminus to carboxy-terminus, represented by:
VL1-L1-VH1 or VH1-L1-VL1;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; and
L1 is an optional amino acid linker.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, ILSR, IL6, IL7, IL7Ra, IL8, IL9, IL9R, IL1A, IL-llRa, integrin α4, integrin α4 β7, integrin α5β1, integrin αIIbβ3, integrin αvβ3, integrin 7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70)-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptides provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae. Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans. Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptides provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptides provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanczumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptides provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptides provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptides provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptides provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides provided herein further comprise an Fc region. In some aspects, the antigen binding polypeptides provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, and T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides disclosed herein are comprised in IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides disclosed herein comprise one or more Fc effector function knockout mutation. In some aspects, the antigen binding polypeptides disclosed herein are comprised in IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides disclosed herein comprise a mutation at a glycosylation site. In some aspects, the mutation at a glycosylation site is a mutation at an N-glycosylation site. In some aspects, the antigen binding polypeptides comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, the antigen binding polypeptides disclosed herein, comprise one or more optional linkers. The one or more optional linkers are indicated herein as Ln, wherein n is an integer, and the integer next to each L indicates each L comprised in the antigen binding polypeptides disclosed herein. For example, if an antigen binding polypeptide disclosed herein comprises two linkers, the first linker from N-terminus to C-terminus is herein indicated as L1, and the second linker from N-terminus to C-terminus is herein indicated as L2. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, an antigen binding polypeptide complex provided herein comprises a first polypeptide, and a second polypeptide; wherein the first polypeptide has a structure represented by VL1-CL-VH1-CH1, VL1-CH1-VH1-CL, VH1-CL-VL1-CH1, or VH1-CH1-VL1-CL; and wherein the second polypeptide has a structure represented by VL2-CL-VL3-VH3-VH2-CH1, VL2-CL-VH3-VL3-VH2-CH1, VL2-CH1-VL3-VH3-VH2-CL, VL2-CH1-VH3-VL3-VH2-CL, VH2-CL-VL3-VH3-VL2-CH1, VH2-CL-VH3-VL3-VL2-CH1, VH2-CH1-VL3-VH3-VL2-CL, VH2-CH1-VH3-VL3-VL2-CL, VL3-CL-VL2-VH2-VH3-CH1, VL3-CL-VH2-VL2-VH3-CH1, VL3-CH1-VL2-VH2-VH3-CL, VL3-CH1-VH2-VL2-VH3-CL, VH3-CL-VL2-VH2-VL3-CH1, VH3-CL-VH2-VL2-VL3-CH1, VH3-CH1-VL2-VH2-VL3-CL, or VH3-CH1-VH2-VL2-VL3-CL.

In some aspects, an antigen binding polypeptide complex provided herein comprises a first polypeptide, and a second polypeptide; wherein the first polypeptide has a structure represented by VL1-L1-CL-L2-VH1-L3-CH1, VL1-L1-CH1-L2-VH1-L3-CL, VH1-L1-CL-L2-VL1-L3-CH1, or VH1-L1- CH1-L2-VL1-L3-CL; and wherein the second polypeptide has a structure represented by VL2-L4-CL-L5-VL3-L6-VH3-L7-VH2-L8-CH1, VL2-L4-CL-L5-VH3-L6-VL3-L7-VH2-L8-CH1, VL2-L4-CH1-L5- VL3-L6-VH3-L7-VH2-L8-CL, VL2-L4-CH1-L5-VH3-L6-VL3-L7-VH2-L8-CL, VH2-L4-CL-L5-VL3-L6-VH3-L7-VL2-L8-CH1, VH2-L4-CL-L5-VH3-L6-VL3-L7-VL2-L8-CH1, VH2-L4-CH1-L5-VL3-L6- VH3-L7-VL2-L8-CL, VH2-L4-CH1-L5-VH3-L6-VL3-L7-VL2-L8-CL, VL3-L4-CL-L5-VL2-L6-VH2-L7-VH3-L8-CH1, VL3-L4-CL-L5-VH2-L6-VL2-L7-VH3-L8-CH1, VL3-L4-CH1-L5-VL2-L6-VH2-L7-VH3-L8-CL, VL3-L4-CH1-L5-VH2-L6-VL2-L7-VH3-L8-CL, VH3-L4-CL-L5-VL2-L6-VH2-L7-VL3-L8-CH1, VH3-L4-CL-L5-VH2-L6-VL2-L7-VL3-L8-CH1, VH3-L4-CH1-L5-VL2-L6-VH2-L7-VL3-L8-CL, or VH3-L4-CH1-L5-VH2-L6-VL2-L7-VL3-L8-CL.

In some aspects, an antigen binding polypeptide complex provided herein comprises a first polypeptide, and a second polypeptide; wherein the first polypeptide has a structure represented by VL1-CL-VH1-CH1-CH2-CH3, VL1-CH1-VH1-CL-CH2-CH3, VH1-CL-VL1-CH1-CH2-CH3, or VH1-CH1-VL1-CL-CH2-CH3; and wherein the second polypeptide has a structure represented by VL2-CL-VL3-VH3-VH2-CH1-CH2-CH3, VL2-CL-VH3-VL3-VH2-CH1-CH2-CH3, VL2-CH1-VL3-VH3-VH2-CL-CH2-CH3, VL2-CH1-VH3-VL3-VH2-CL-CH2-CH3, VH2-CL-VL3-VH3-VL2-CH1-CH2-CH3, VH2-CL-VH3-VL3-VL2-CH1-CH2-CH3, VH2-CH1-VL3-VH3-VL2-CL-CH2-CH3, VH2-CH1-VH3-VL3-VL2-CL-CH2-CH3, VL3-CL-VL2-VH2-VH3-CH1-CH2-CH3, VL3-CL-VH2-VL2-VH3-CH1-CH2-CH3, VL3-CH1-VL2-VH2-VH3-CL-CH2-CH3, VL3-CH1-VH2-VL2-VH3-CL-CH2-CH3, VH3-CL-VL2-VH2-VL3-CH1-CH2-CH3, VH3-CL-VH2-VL2-VL3-CH1-CH2-CH3, VH3-CH1-VL2-VH2-VL3-CL-CH2-CH3, or VH3-CH1-VH2-VL2-VL3-CL-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-CH2-CH3, VL1-L1-CH1-L2-VH1-L3-CL-CH2-CH3, VH1-L1-CL-L2-VL1- L3-CH1-CH2-CH3, or VH1-L1-CH1-L2-VL1-L3-CL-CH2-CH3; and wherein the second polypeptide has a structure represented by VL2-L4-CL-L5-VL3-L6-VH3-L7-VH2-L8-CH1-CH2-CH3, VL2-L4-CL-L5-VH3-L6-VL3-L7-VH2-L8-CH1-CH2-CH3, VL2-L4-CH1-L5-VL3-L6-VH3-L7-VH2-L8-CL-CH2-CH3, VL2-L4-CH1-L5-VH3-L6-VL3-L7-VH2-L8-CL-CH2-CH3, VH2-L4-CL-L5-VL3-L6-VH3-L7-VL2-L8-CH1-CH2-CH3, VH2-L4-CL-L5-VH3-L6-VL3-L7-VL2-L8-CH1-CH2-CH3, VH2-L4-CH1-L5-VL3-L6-VH3-L7-VL2-L8-CL-CH2-CH3, VH2-L4-CH1-L5-VH3-L6-VL3-L7-VL2-L8-CL-CH2-CH3, VL3-L4-CL-L5-VL2-L6-VH2-L7-VH3-L8-CH1-CH2-CH3, VL3-L4-CL-L5-VH2-L6-VL2-L7-VH3-L8-CH1-CH2-CH3, VL3-L4-CH1-L5-VL2-L6-VH2-L7-VH3-L8-CL-CH2-CH3, VL3-L4-CH1-L5-VH2-L6-VL2-L7-VH3-L8-CL-CH2-CH3, VH3-L4-CL-L5-VL2-L6-VH2-L7-VL3-L8-CH1-CH2-CH3, VH3-L4-CL-L5-VH2-L6-VL2-L7-VL3-L8-CH1-CH2-CH3, VH3-L4-CH1-L5-VL2-L6-VH2-L7-VL3-L8-CL-CH2-CH3, or VH3-L4-CH1-L5-VH2-L6-VL2-L7-VL3-L8-CL-CH2-CH3.

In some aspects, an antigen binding polypeptide complex provided herein comprises a first polypeptide, and a second polypeptide; wherein the first polypeptide has a structure represented by VL1-CL-VH1-CH1, VL1-CH1-VH1-CL, VH1-CL-VL1-CH1, or VH1-CH1-VL1-CL; and wherein the second polypeptide has a structure represented by VL2-VH2-VL3-CL-VH3-CH1, VL2-VH2-VL3-CH1-VH3-CL, VL2-VH2-VH3-CL-VL3-CH1, VL2-VH2-VH3-CH1-VL3-CL, VL2-VL3-VH2-CL-VH3-CH1, VL2-VL3-VH2-CH1-VH3-CL, VL2-VH3-VH2-CL-VL3-CH1, VL2-VH3-VH2-CH1-VL3-CL, VH2-VL2-VL3-CL-VH3-CH1, VH2-VL2-VL3-CH1-VH3-CL, VH2-VL2-VH3-CL-VL3-CH1, VH2-VL2-VH3-CH1-VL3-CL, VH2-VL3-VL2-CL-VH3-CH1, VH2-VL3-VL2-CH1-VH3-CL, VH2-VH3-VL2-CL-VL3-CH1, VH2-VH3-VL2-CH1-VL3-CL, VL3-VL2-VH3-CL-VH2-CH1, VL3-VL2-VH3-CH1-VH2-CL, VL3-VH2-VH3-CL-VL2-CH1, VL3-VH2-VH3-CH1-VL2-CL, VL3-VH3-VL2-CL-VH2-CH1, VL3-VH3-VL2-CH1-VH2-CL, VL3-VH3-VH2-CL-VL2-CH1, VL3-VH3-VH2-CH1-VL2-CL, VH3-VL2-VL3-CL-VH2-CH1, VH3-VL2-VL3-CH1-VH2-CL, VH3-VH2-VL3-CL-VL2-CH1, VH3-VH2-VL3-CH1-VL2-CL, VH3-VL3-VL2-CL-VH2-CH1, VH3-VL3-VL2-CH1-VH2-CL, VH3-VL3-VH2-CL-VL2-CH1, or VH3-VL3-VH2-CH1-VL2-CL.

In some aspects, an antigen binding polypeptide complex provided herein comprises a first polypeptide, and a second polypeptide; wherein the first polypeptide has a structure represented by VL1-L1-CL-L2-VH1-L3-CH1, VL1-L1-CH1-L2-VH1-L3-CL, VH1-L1-CL-L2-VL1-L3-CH1, or VH1-L1- CH1-L2-VL1-L3-CL; and wherein the second polypeptide has a structure represented by VL2-L4-VH2-L5-VL3-L6-CL-L7-VH3-L8-CH1, VL2-L4-VH2-L5-VL3-L6-CH1-L7-VH3-L8-CL, VL2-L4-VH2-L5-VH3-L6-CL-L7-VL3-L8-CH1, VL2-L4-VH2-L5-VH3-L6-CH1-L7-VL3-L8-CL, VL2-L4-VL3-L5-VH2-L6-CL-L7-VH3-L8-CH1, VL2-L4-VL3-L5-VH2-L6-CH1-L7-VH3-L8-CL, VL2-L4-VH3-L5-VH2-L6-CL-L7-VL3-L8-CH1, VL2-L4-VH3-L5-VH2-L6-CH1-L7-VL3-L8-CL, VH2-L4-VL2-L5-VL3-L6-CL-L7-VH3-L8-CH1, VH2-L4-VL2-L5-VL3-L6-CH1-L7-VH3-L8-CL, VH2-L4-VL2-L5-VH3-L6-CL-L7-VL3-L8-CH1, VH2-L4-VL2-L5-VH3-L6-CH1-L7-VL3-L8-CL, VH2-L4-VL3-L5-VL2-L6-CL-L7-VH3-L8-CH1, VH2-L4-VL3-L5-VL2-L6-CH1-L7-VH3-L8-CL, VH2-L4-VH3-L5-VL2-L6-CL-L7-VL3-L8-CH1, VH2-L4-VH3-L5-VL2-L6-CH1-L7-VL3-L8-CL, VL3-L4-VL2-L5-VH3-L6-CL-L7-VH2-L8-CH1, VL3-L4-VL2-L5-VH3-L6-CH1-L7-VH2-L8-CL, VL3-L4-VH2-L5-VH3-L6-CL-L7-VL2-L8-CH1, VL3-L4-VH2-L5-VH3-L6-CH1-L7-VL2-L8-CL, VL3-L4-VH3-L5-VL2-L6-CL-L7-VH2-L8-CH1, VL3-L4-VH3-L5-VL2-L6-CH1-L7-VH2-L8-CL, VL3-L4-VH3-L5-VH2-L6-CL-L7-VL2-L8-CH1, VL3-L4-VH3-L5-VH2-L6-CH1-L7-VL2-L8-CL, VH3-L4-VL2-L5-VL3-L6-CL-L7-VH2-L8-CH1, VH3-L4-VL2-L5-VL3-L6-CH1-L7-VH2-L8-CL, VH3-L4-VH2-L5-VL3-L6-CL-L7-VL2-L8-CH1, VH3-L4-VH2-L5-VL3-L6-CH1-L7-VL2-L8-CL, VH3-L4-VL3-L5-VL2-L6-CL-L7-VH2-L8-CH1, VH3-L4-VL3-L5-VL2-L6-CH1-L7-VH2-L8-CL, VH3-L4-VL3-L5-VH2-L6-CL-L7-VL2-L8-CH1, or VH3-L4-VL3-L5-VH2-L6-CH1-L7-VL2-L8-CL.

In some aspects, an antigen binding polypeptide complex provided herein comprises a first polypeptide, and a second polypeptide; wherein the first polypeptide has a structure represented by VL1-CL-VH1-CH1-CH2-CH3, VL1-CH1-VH1-CL-CH2-CH3, VH1-CL-VL1-CH1-CH2-CH3, or VH1-CH1-VL1-CL-CH2-CH3; and wherein the second polypeptide has a structure represented by VL2-VH2-VL3-CL-VH3-CH1-CH2-CH3, VL2-VH2-VL3-CH1-VH3-CL-CH2-CH3, VL2-VH2-VH3-CL-VL3-CH1-CH2-CH3, VL2-VH2-VH3-CH1-VL3-CL-CH2-CH3, VL2-VL3-VH2-CL-VH3-CH1-CH2-CH3, VL2-VL3-VH2-CH1-VH3-CL-CH2-CH3, VL2-VH3-VH2-CL-VL3-CH1-CH2-CH3, VL2-VH3-VH2-CH1-VL3-CL-CH2-CH3, VH2-VL2-VL3-CL-VH3-CH1-CH2-CH3, VH2-VL2-VL3-CH1-VH3-CL-CH2-CH3, VH2-VL2-VH3-CL-VL3-CH1-CH2-CH3, VH2-VL2-VH3-CH1-VL3-CL-CH2-CH3, VH2-VL3-VL2-CL-VH3-CH1-CH2-CH3, VH2-VL3-VL2-CH1-VH3-CL-CH2-CH3, VH2-VH3-VL2-CL-VL3-CH1-CH2-CH3, VH2-VH3-VL2-CH1-VL3-CL-CH2-CH3, VL3-VL2-VH3-CL-VH2-CH1-CH2-CH3, VL3-VL2-VH3-CH1-VH2-CL-CH2-CH3, VL3-VH2-VH3-CL-VL2-CH1-CH2-CH3, VL3-VH2-VH3-CH1-VL2-CL-CH2-CH3, VL3-VH3-VL2-CL-VH2-CH1-CH2-CH3, VL3-VH3-VL2-CH1-VH2-CL-CH2-CH3, VL3-VH3-VH2-CL-VL2-CH1-CH2-CH3, VL3-VH3-VH2-CH1-VL2-CL-CH2-CH3, VH3-VL2-VL3-CL-VH2-CH1-CH2-CH3, VH3-VL2-VL3-CH1-VH2-CL-CH2-CH3, VH3-VH2-VL3-CL-VL2-CH1-CH2-CH3, VH3-VH2-VL3-CH1-VL2-CL-CH2-CH3, VH3-VL3-VL2-CL-VH2-CH1-CH2-CH3, VH3-VL3-VL2-CH1-VH2-CL-CH2-CH3, VH3-VL3-VH2-CL-VL2-CH1-CH2-CH3, or VH3-VL3-VH2-CH1-VL2-CL-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-CH2-CH3, VL1-L1-CH1-L2-VH1-L3-CL-CH2-CH3, VH1-L1-CL-L2-VL1- L3-CH1-CH2-CH3, or VH1-L1-CH1-L2-VL1-L3-CL-CH2-CH3; and wherein the second polypeptide has a structure represented by VL2-L4-VH2-L5-VL3-L6-CL-L7-VH3-L8-CH1-CH2-CH3, VL2-L4-VH2-L5-VL3-L6-CH1-L7-VH3-L8-CL-CH2-CH3, VL2-L4-VH2-L5-VH3-L6-CL-L7-VL3-L8-CH1-CH2-CH3, VL2-L4-VH2-L5-VH3-L6-CH1-L7-VL3-L8-CL-CH2-CH3, VL2-L4-VL3-L5-VH2-L6-CL-L7-VH3-L8-CH1-CH2-CH3, VL2-L4-VL3-L5-VH2-L6-CH1-L7-VH3-L8-CL-CH2-CH3, VL2-L4-VH3-L5-VH2-L6-CL-L7-VL3-L8-CH1-CH2-CH3, VL2-L4-VH3-L5-VH2-L6-CH1-L7-VL3-L8-CL-CH2-CH3, VH2-L4-VL2-L5-VL3-L6-CL-L7-VH3-L8-CH1-CH2-CH3, VH2-L4-VL2-L5-VL3-L6-CH1-L7-VH3-L8- CL-CH2-CH3, VH2-L4-VL2-L5-VH3-L6-CL-L7-VL3-L8-CH1-CH2-CH3, VH2-L4-VL2-L5-VH3-L6-CH1-L7-VL3-L8-CL-CH2-CH3, VH2-L4-VL3-L5-VL2-L6-CL-L7-VH3-L8-CH1-CH2-CH3, VH2-L4-VL3-L5-VL2-L6-CH1-L7-VH3-L8-CL-CH2-CH3, VH2-L4-VH3-L5-VL2-L6-CL-L7-VL3-L8-CH1-CH2-CH3, VH2-L4-VH3-L5-VL2-L6-CH1-L7-VL3-L8-CL-CH2-CH3, VL3-L4-VL2-L5-VH3-L6-CL-L7-VH2-L8-CH1-CH2-CH3, VL3-L4-VL2-L5-VH3-L6-CH1-L7-VH2-L8-CL-CH2-CH3, VL3-L4-VH2-L5-VH3-L6-CL-L7-VL2-L8-CH1-CH2-CH3, VL3-L4-VH2-L5-VH3-L6-CH1-L7-VL2-L8-CL-CH2-CH3, VL3-L4-VH3-L5-VL2-L6-CL-L7-VH2-L8-CH1-CH2-CH3, VL3-L4-VH3-L5-VL2-L6-CH1-L7-VH2-L8- CL-CH2-CH3, VL3-L4-VH3-L5-VH2-L6-CL-L7-VL2-L8-CH1-CH2-CH3, VL3-L4-VH3-L5-VH2-L6-CH1-L7-VL2-L8-CL-CH2-CH3, VH3-L4-VL2-L5-VL3-L6-CL-L7-VH2-L8-CH1-CH2-CH3, VH3-L4-VL2-L5-VL3-L6-CH1-L7-VH2-L8-CL-CH2-CH3, VH3-L4-VH2-L5-VL3-L6-CL-L7-VL2-L8-CH1-CH2-CH3, VH3-L4-VH2-L5-VL3-L6-CH1-L7-VL2-L8-CL-CH2-CH3, VH3-L4-VL3-L5-VL2-L6-CL-L7-VH2-L8-CH1-CH2-CH3, VH3-L4-VL3-L5-VL2-L6-CH1-L7-VH2-L8-CL-CH2-CH3, VH3-L4-VL3-L5-VH2-L6-CL-L7-VL2-L8-CH1-CH2-CH3, or VH3-L4-VL3-L5-VH2-L6-CH1-L7-VL2-L8-CL-CH2-CH3.

In some aspects, an antigen binding polypeptide complex provided herein comprises a first polypeptide, and a second polypeptide; wherein the first polypeptide has a structure represented by VL1-CL-VH1-CH1, VL1-CH1-VH1-CL, VH1-CL-VL1-CH1, or VH1-CH1-VL1-CL; and wherein the second polypeptide has a structure represented by VL2-CL-VH2-CH1-VL3-VH3, VL2-CL-VH2-CH1-VH3-VL3, VL2-CL-VL3-CH1-VH2-VH3, VL2-CL-VH3-CH1-VH2-VL3, VL2-CH1-VH2-CL-VL3-VH3, VL2-CH1-VH2-CL-VH3-VL3, VL2-CH1-VL3-CL-VH2-VH3, VL2-CH1-VH3-CL-VH2-VL3, VH2-CL-VL2-CH1-VL3-VH3, VH2-CL-VL2-CH1-VH3-VL3, VH2-CL-VL3-CH1-VL2-VH3, VH2-CL-VH3-CH1-VL2-VL3, VH2-CH1-VL2-CL-VL3-VH3, VH2-CH1-VL2-CL-VH3-VL3, VH2-CH1-VL3-CL-VL2-VH3, VH2-CH1-VH3-CL-VL2-VL3, VL3-CL-VL2-CH1-VH3-VH2, VL3-CL-VH2-CH1-VH3-VL2, VL3-CL-VH3-CH1-VL2-VH2, VL3-CL-VH3-CH1-VH2-VL2, VL3-CH1-VL2-CL-VH3-VH2, VL3-CH1-VH2-CL-VH3-VL2, VL3-CH1-VH3-CL-VL2-VH2, VL3-CH1-VH3-CL-VH2-VL2, VH3-CL-VL2-CH1-VL3-VH2, VH3-CL-VH2-CH1-VL3-VL2, VH3-CL-VL3-CH1-VL2-VH2, VH3-CL-VL3-CH1-VH2-VL2, VH3-CH1-VL2-CL-VL3-VH2, VH3-CH1-VH2-CL-VL3-VL2, VH3-CH1-VL3-CL-VL2-VH2, or VH3-CH1-VL3-CL-VH2-VL2.

In some aspects, an antigen binding polypeptide complex provided herein comprises a first polypeptide, and a second polypeptide; wherein the first polypeptide has a structure represented by VL1-L1-CL-L2-VH1-L3-CH1, VL1-L1-CH1-L2-VH1-L3-CL, VH1-L1-CL-L2-VL1-L3-CH1, or VH1-L1- CH1-L2-VL1-L3-CL; and wherein the second polypeptide has a structure represented by VL2-L4-CL-L5-VH2-L6-CH1-L7-VL3-L8-VH3, VL2-L4-CL-L5-VH2-L6-CH1-L7-VH3-L8-VL3, VL2-L4-CL-L5-VL3-L6-CH1-L7-VH2-L8-VH3, VL2-L4-CL-L5-VH3-L6-CH1-L7-VH2-L8-VL3, VL2-L4-CH1-L5-VH2-L6-CL-L7-VL3-L8-VH3, VL2-L4-CH1-L5-VH2-L6-CL-L7-VH3-L8-VL3, VL2-L4-CH1-L5-VL3-L6-CL- L7-VH2-L8-VH3, VL2-L4-CH1-L5-VH3-L6-CL-L7-VH2-L8-VL3, VH2-L4-CL-L5-VL2-L6-CH1-L7-VL3-L8-VH3, VH2-L4-CL-L5-VL2-L6-CH1-L7-VH3-L8-VL3, VH2-L4-CL-L5-VL3-L6-CH1-L7-VL2-L8-VH3, VH2-L4-CL-L5-VH3-L6-CH1-L7-VL2-L8-VL3, VH2-L4-CH1-L5-VL2-L6-CL-L7-VL3-L8-VH3, VH2-L4-CH1-L5-VL2-L6-CL-L7-VH3-L8-VL3, VH2-L4-CH1-L5-VL3-L6-CL-L7-VL2-L8-VH3, VH2-L4-CH1-L5-VH3-L6-CL-L7-VL2-L8-VL3, VL3-L4-CL-L5-VL2-L6-CH1-L7-VH3-L8-VH2, VL3-L4-CL-L5-VH2-L6-CH1-L7-VH3-L8-VL2, VL3-L4-CL-L5-VH3-L6-CH1-L7-VL2-L8-VH2, VL3-L4-CL- L5-VH3-L6-CH1-L7-VH2-L8-VL2, VL3-L4-CH1-L5-VL2-L6-CL-L7-VH3-L8-VH2, VL3-L4-CH1-L5-VH2-L6-CL-L7-VH3-L8-VL2, VL3-L4-CH1-L5-VH3-L6-CL-L7-VL2-L8-VH2, VL3-L4-CH1-L5-VH3-L6-CL-L7-VH2-L8-VL2, VH3-L4-CL-L5-VL2-L6-CH1-L7-VL3-L8-VH2, VH3-L4-CL-L5-VH2-L6-CH1- L7-VL3-L8-VL2, VH3-L4-CL-L5-VL3-L6-CH1-L7-VL2-L8-VH2, VH3-L4-CL-L5-VL3-L6-CH1-L7-VH2-L8-VL2, VH3-L4-CH1-L5-VL2-L6-CL-L7-VL3-L8-VH2, VH3-L4-CH1-L5-VH2-L6-CL-L7-VL3-L8-VL2, VH3-L4-CH1-L5-VL3-L6-CL-L7-VL2-L8-VH2, or VH3-L4-CH1-L5-VL3-L6-CL-L7-VH2-L8-VL2.

In some aspects, an antigen binding polypeptide complex provided herein comprises a first polypeptide, and a second polypeptide; wherein the first polypeptide has a structure represented by VL1-CL-VH1-CH1-CH2-CH3, VL1-CH1-VH1-CL-CH2-CH3, VH1-CL-VL1-CH1-CH2-CH3, or VH1-CH1-VL1-CL-CH2-CH3; and wherein the second polypeptide has a structure represented by VL2-CL-VH2-CH1-VL3-VH3-CH2-CH3, VL2-CL-VH2-CH1-VH3-VL3-CH2-CH3, VL2-CL-VL3-CH1-VH2-VH3-CH2-CH3, VL2-CL-VH3-CH1-VH2-VL3-CH2-CH3, VL2-CH1-VH2-CL-VL3-VH3-CH2-CH3, VL2-CH1-VH2-CL-VH3-VL3-CH2-CH3, VL2-CH1-VL3-CL-VH2-VH3-CH2-CH3, VL2-CH1-VH3-CL-VH2-VL3-CH2-CH3, VH2-CL-VL2-CH1-VL3-VH3-CH2-CH3, VH2-CL-VL2-CH1-VH3-VL3-CH2-CH3, VH2-CL-VL3-CH1-VL2-VH3-CH2-CH3, VH2-CL-VH3-CH1-VL2-VL3-CH2-CH3, VH2-CH1-VL2-CL-VL3-VH3-CH2-CH3, VH2-CH1-VL2-CL-VH3-VL3-CH2-CH3, VH2-CH1-VL3-CL-VL2-VH3-CH2-CH3, VH2-CH1-VH3-CL-VL2-VL3-CH2-CH3, VL3-CL-VL2-CH1-VH3-VH2-CH2-CH3, VL3-CL-VH2-CH1-VH3-VL2-CH2-CH3, VL3-CL-VH3-CH1-VL2-VH2-CH2-CH3, VL3-CL-VH3-CH1-VH2-VL2-CH2-CH3, VL3-CH1-VL2-CL-VH3-VH2-CH2-CH3, VL3-CH1-VH2-CL-VH3-VL2-CH2-CH3, VL3-CH1-VH3-CL-VL2-VH2-CH2-CH3, VL3-CH1-VH3-CL-VH2-VL2-CH2-CH3, VH3-CL-VL2-CH1-VL3-VH2-CH2-CH3, VH3-CL-VH2-CH1-VL3-VL2-CH2-CH3, VH3-CL-VL3-CH1-VL2-VH2-CH2-CH3, VH3-CL-VL3-CH1-VH2-VL2-CH2-CH3, VH3-CH1-VL2-CL-VL3-VH2-CH2-CH3, VH3-CH1-VH2-CL-VL3-VL2-CH2-CH3, VH3-CH1-VL3-CL-VL2-VH2-CH2-CH3, or VH3-CH1-VL3-CL-VH2-VL2-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-CH2-CH3, VL1-L1-CH1-L2-VH1-L3-CL-CH2-CH3, VH1-L1-CL-L2-VL1- L3-CH1-CH2-CH3, or VH1-L1-CH1-L2-VL1-L3-CL-CH2-CH3; and wherein the second polypeptide has a structure represented by VL2-L4-CL-L5-VH2-L6-CH1-L7-VL3-L8-VH3-CH2-CH3, VL2-L4-CL-L5-VH2-L6-CH1-L7-VH3-L8-VL3-CH2-CH3, VL2-L4-CL-L5-VL3-L6-CH1-L7-VH2-L8-VH3-CH2-CH3, VL2-L4-CL-L5-VH3-L6-CH1-L7-VH2-L8-VL3-CH2-CH3, VL2-L4-CH1-L5-VH2-L6-CL-L7-VL3-L8-VH3-CH2-CH3, VL2-L4-CH1-L5-VH2-L6-CL-L7-VH3-L8-VL3-CH2-CH3, VL2-L4-CH1-L5-VL3-L6-CL-L7-VH2-L8-VH3-CH2-CH3, VL2-L4-CH1-L5-VH3-L6-CL-L7-VH2-L8-VL3-CH2-CH3, VH2-L4-CL-L5-VL2-L6-CH1-L7-VL3-L8-VH3-CH2-CH3, VH2-L4-CL-L5-VL2-L6-CH1-L7-VH3-L8-VL3-CH2-CH3, VH2-L4-CL-L5-VL3-L6-CH1-L7-VL2-L8-VH3-CH2-CH3, VH2-L4-CL-L5-VH3-L6-CH1-L7-VL2-L8-VL3-CH2-CH3, VH2-L4-CH1-L5-VL2-L6-CL-L7-VL3-L8-VH3-CH2-CH3, VH2-L4-CH1-L5-VL2-L6-CL-L7-VH3-L8-VL3-CH2-CH3, VH2-L4-CH1-L5-VL3-L6-CL-L7-VL2-L8-VH3-CH2-CH3, VH2-L4-CH1-L5-VH3-L6-CL-L7-VL2-L8-VL3-CH2-CH3, VL3-L4-CL-L5-VL2-L6-CH1-L7-VH3-L8-VH2-CH2-CH3, VL3-L4-CL-L5-VH2-L6-CH1-L7-VH3-L8-VL2-CH2-CH3, VL3-L4-CL-L5-VH3-L6-CH1-L7-VL2-L8-VH2-CH2-CH3, VL3-L4-CL-L5-VH3-L6-CH1-L7-VH2-L8-VL2-CH2-CH3, VL3-L4-CH1-L5-VL2-L6-CL-L7-VH3-L8-VH2-CH2-CH3, VL3-L4-CH1-L5-VH2-L6-CL-L7-VH3-L8-VL2-CH2-CH3, VL3-L4-CH1-L5-VH3-L6-CL-L7-VL2-L8-VH2-CH2-CH3, VL3-L4-CH1-L5-VH3-L6-CL-L7-VH2-L8-VL2-CH2-CH3, VH3-L4-CL-L5-VL2-L6-CH1-L7-VL3-L8-VH2-CH2-CH3, VH3-L4-CL-L5-VH2-L6-CH1-L7-VL3-L8-VL2-CH2-CH3, VH3-L4-CL-L5-VL3-L6-CH1-L7-VL2-L8-VH2-CH2-CH3, VH3-L4-CL-L5-VL3-L6-CH1-L7-VH2-L8-VL2-CH2-CH3, VH3-L4-CH1-L5-VL2-L6-CL-L7-VL3-L8-VH2-CH2-CH3, VH3-L4-CH1-L5-VH2-L6-CL-L7-VL3-L8-VL2-CH2-CH3, VH3-L4-CH1-L5-VL3-L6-CL-L7-VL2-L8-VH2-CH2-CH3, or VH3-L4-CH1-L5-VL3-L6-CL-L7-VH2-L8-VL2-CH2-CH3.

In some aspects, VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; CH1 is an immunoglobulin heavy chain constant region 1; CL is an immunoglobulin light chain constant region; and L1-L8 are optional amino acid linkers. In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the VL1 and the VL2 each comprise a CDR1, a CDR2, and a CDR3 that are the same. In some aspects, the VL1 and the VL2 each comprise a CDR1, a CDR2, and a CDR3 that are different. In some aspects, the VL1 and the VL3 each comprise a CDR1, a CDR2, and a CDR3 that are the same. In some aspects, the VL1 and the VL3 each comprise a CDR1, a CDR2, and a CDR3 that are different. In some aspects, the VL2 and the VL3 each comprise a CDR1, a CDR2, and a CDR3 that are the same. In some aspects, the VL2 and the VL3 each comprise a CDR1, a CDR2, and a CDR3 that are different.

In some aspects, the VL1 and the VL2 each comprise an amino acid sequence, wherein the amino acid sequence comprised in VL1 and the amino acid sequence comprised in VL2 are the same. In some aspects, the VL1 and the VL2 each comprise an amino acid sequence, wherein the amino acid sequence comprised in VL1 and the amino acid sequence comprised in VL2 are different. In some aspects, the VL1 and the VL3 each comprise an amino acid sequence, wherein the amino acid sequence comprised in VL1 and the amino acid sequence comprised in VL3 are the same. In some aspects, the VL1 and the VL3 each comprise an amino acid sequence, wherein the amino acid sequence comprised in VL1 and the amino acid sequence comprised in VL3 are different. In some aspects, the VL2 and the VL3 each comprise an amino acid sequence, wherein the amino acid sequence comprised in VL2 and the amino acid sequence comprised in VL3 are the same. In some aspects, the VL2 and the VL3 each comprise an amino acid sequence, wherein the amino acid sequence comprised in VL2 and the amino acid sequence comprised in VL3 are different.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, ILSR, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90 granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptide complexes provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL3 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, crtumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH3 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL3 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezckimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH3 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozclimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptide complexes provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or

(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptide complexes provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL3 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH3 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL2 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL3 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH3 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOS: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOS: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptide complexes provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptide complexes provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL3 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, clezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH3 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elevanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL3 comprising a VL of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH3 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptide complexes provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, an antigen binding polypeptide complex provided herein comprises a first polypeptide, and a second polypeptide; wherein the first polypeptide has a structure represented by VL1-CL-VL2-VH2-VH1-CH1, VL1-CL-VH2-VL2-VH1-CH1, VL1-CH1-VL2-VH2-VH1-CL, VL1-CH1-VH2-VL2-VH1-CL, VH1-CL-VL2-VH2-VL1-CH1, VH1-CL-VH2-VL2-VL1-CH1, VH1-CH1-VL2-VH2-VL1-CL, VH1-CH1-VH2-VL2-VL1-CL, VL2-CL-VL1-VH1-VH2-CH1, VL2-CL-VH1-VL1-VH2-CH1, VL2-CH1-VL1-VH1-VH2-CL, VL2-CH1-VH1-VL1-VH2-CL, VH2-CL-VL1-VH1-VL2-CH1, VH2-CL-VH1-VL1-VL2-CH1, VH2-CH1-VL1-VH1-VL2-CL, or VH2-CH1-VH1-VL1-VL2-CL; and wherein the second polypeptide has a structure represented by VL3-CL-VL4-VH4-VH3-CH1, VL3-CL-VH4-VL4-VH3-CH1, VL3-CH1-VL4-VH4-VH3-CL, VL3-CH1-VH4-VL4-VH3-CL, VH3-CL-VL4-VH4-VL3-CH1, VH3-CL-VH4-VL4-VL3-CH1, VH3-CH1-VL4-VH4-VL3-CL, VH3-CH1-VH4-VL4-VL3-CL, VL4-CL-VL3-VH3-VH4-CH1, VL4-CL-VH3-VL3-VH4-CH1, VL4-CH1-VL3-VH3-VH4-CL, VL4-CH1-VH3-VL3-VH4-CL, VH4-CL-VL3-VH3-VL4-CH1, VH4-CL-VH3-VL3-VL4-CH1, VH4-CH1-VL3-VH3-VL4-CL, or VH4-CH1-VH3-VL3-VL4-CL.

In some aspects, an antigen binding polypeptide complex provided herein comprises a first polypeptide, and a second polypeptide; wherein the first polypeptide has a structure represented by VL1-L1-CL-L2-VL2-L3-VH2-L4-VH1-L5-CH1, VL1-L1-CL-L2-VH2-L3-VL2-L4-VH1-L5-CH1, VL1-L1-CH1-L2-VL2-L3-VH2-L4-VH1-L5-CL, VL1-L1-CH1-L2-VH2-L3-VL2-L4-VH1-L5-CL, VH1-L1-CL-L2-VL2-L3-VH2-L4-VL1-L5-CH1, VH1-L1-CL-L2-VH2-L3-VL2-L4-VL1-L5-CH1, VH1-L1-CH1-L2-VL2-L3-VH2-L4-VL1-L5-CL, VH1-L1-CH1-L2-VH2-L3-VL2-L4-VL1-L5-CL, VL2-L1-CL-L2-VL1-L3-VH1-L4-VH2-L5-CH1, VL2-L1-CL-L2-VH1-L3-VL1-L4-VH2-L5-CH1, VL2-L1-CH1-L2-VL1-L3-VH1-L4-VH2-L5-CL, VL2-L1-CH1-L2-VH1-L3-VL1-L4-VH2-L5-CL, VH2-L1-CL-L2-VL1-L3-VH1-L4-VL2-L5-CH1, VH2-L1-CL-L2-VH1-L3-VL1-L4-VL2-L5-CH1, VH2-L1-CH1-L2-VL1-L3-VH1-L4- VL2-L5-CL, or VH2-L1-CH1-L2-VH1-L3-VL1-L4-VL2-L5-CL; and wherein the second polypeptide has a structure represented by VL3-L6-CL-L7-VL4-L8-VH4-L9-VH3-L10-CH1, VL3-L6-CL-L7-VH4-L8-VL4-L9-VH3-L10-CH1, VL3-L6-CH1-L7-VL4-L8-VH4-L9-VH3-L10-CL, VL3-L6-CH1-L7-VH4-L8-VL4-L9-VH3-L10-CL, VH3-L6-CL-L7-VL4-L8-VH4-L9-VL3-L10-CH1, VH3-L6-CL-L7-VH4-L8-VL4-L9- VL3-L10-CH1, VH3-L6-CH1-L7-VL4-L8-VH4-L9-VL3-L10-CL, VH3-L6-CH1-L7-VH4-L8-VL4-L9-VL3-L10-CL, VL4-L6-CL-L7-VL3-L8-VH3-L9-VH4-L10-CH1, VL4-L6-CL-L7-VH3-L8-VL3-L9-VH4- L10-CH1, VL4-L6-CH1-L7-VL3-L8-VH3-L9-VH4-L10-CL, VL4-L6-CH1-L7-VH3-L8-VL3-L9-VH4-L10-CL, VH4-L6-CL-L7-VL3-L8-VH3-L9-VL4-L10-CH1, VH4-L6-CL-L7-VH3-L8-VL3-L9-VL4-L10- CH1, VH4-L6-CH1-L7-VL3-L8-VH3-L9-VL4-L10-CL, or VH4-L6-CH1-L7-VH3-L8-VL3-L9-VL4-L10-CL.

In some aspects, an antigen binding polypeptide complex provided herein comprises a first polypeptide, and a second polypeptide; wherein the first polypeptide has a structure represented by VL1-CL-L1-VL2-L2-VH2-L3-VH1-CH1; VL1-CL-L1-VH2-L2-VL2-L3-VH1-CH1; VL1-CH1-L1-VL2-L2-VH2-L3-VH1-CL; VL1-CH1-L1-VH2-L2-VL2-L3-VH1-CL; VH1-CL-L1-VL2-L2-VH2-L3-VL1-CH1; VH1-CL-L1-VH2-L2-VL2-L3-VL1-CH1; VH1-CH1-L1-VL2-L2-VH2-L3-VL1-CL; VH1-CH1-L1-VH2-L2-VL2-L3-VL1-CL; VL2-CL-L1-VL1-L2-VH1-L3-VH2-CH1; VL2-CL-L1-VH1-L2-VL1-L3-VH2- CH1; VL2-CH1-L1-VL1-L2-VH1-L3-VH2-CL; VL2-CH1-L1-VH1-L2-VL1-L3-VH2-CL; VH2-CL-L1-VL1-L2-VH1-L3-VL2-CH1; VH2-CL-L1-VH1-L2-VL1-L3-VL2-CH1; VH2-CH1-L1-VL1-L2-VH1-L3-VL2-CL; or VH2-CH1-L1-VH1-L2-VL1-L3-VL2-CL; and wherein the second polypeptide has a structure represented by VL3-CL-L4-VL4-L5-VH4-L6-VH3-CH1; VL3-CL-L4-VH4-L5-VL4-L6-VH3-CH1; VL3-CH1-L4-VL4-L5-VH4-L6-VH3-CL; VL3-CH1-L4-VH4-L5-VL4-L6-VH3-CL; VH3-CL-L4-VL4-L5-VH4-L6-VL3-CH1; VH3-CL-L4-VH4-L5-VL4-L6-VL3-CH1; VH3-CH1-L4-VL4-L5-VH4-L6-VL3-CL; VH3-CH1-L4-VH4-L5-VL4-L6-VL3-CL; VL4-CL-L4-VL3-L5-VH3-L6-VH4-CH1; VL4-CL-L4-VH3-L5-VL3-L6-VH4-CH1; VL4-CH1-L4-VL3-L5-VH3-L6-VH4-CL; VL4-CH1-L4-VH3-L5-VL3-L6-VH4-CL; VH4-CL-L4-VL3-L5-VH3-L6-VL4-CH1; VH4-CL-L4-VH3-L5-VL3-L6-VL4-CH1; VH4-CH1-L4-VL3-L5-VH3-L6-VL4-CL; or VH4-CH1-L4-VH3-L5-VL3-L6-VL4-CL.

In some aspects, an antigen binding polypeptide complex provided herein comprises a first polypeptide, and a second polypeptide; wherein the first polypeptide has a structure represented by VL1-CL-VL2-VH2-VH1-CH1-CH2-CH3, VL1-CL-VH2-VL2-VH1-CH1-CH2-CH3, VL1-CH1-VL2-VH2-VH1-CL-CH2-CH3, VL1-CH1-VH2-VL2-VH1-CL-CH2-CH3, VH1-CL-VL2-VH2-VL1-CH1-CH2-CH3, VH1-CL-VH2-VL2-VL1-CH1-CH2-CH3, VH1-CH1-VL2-VH2-VL1-CL-CH2-CH3, VH1-CH1-VH2-VL2-VL1-CL-CH2-CH3, VL2-CL-VL1-VH1-VH2-CH1-CH2-CH3, VL2-CL-VH1-VL1-VH2-CH1-CH2-CH3, VL2-CH1-VL1-VH1-VH2-CL-CH2-CH3, VL2-CH1-VH1-VL1-VH2-CL-CH2-CH3, VH2-CL-VL1-VH1-VL2-CH1-CH2-CH3, VH2-CL-VH1-VL1-VL2-CH1-CH2-CH3, VH2-CH1-VL1-VH1-VL2-CL-CH2-CH3, or VH2-CH1-VH1-VL1-VL2-CL-CH2-CH3; and wherein the second polypeptide has a structure represented by VL3-CL-VL4-VH4-VH3-CH1-CH2-CH3, VL3-CL-VH4-VL4-VH3-CH1-CH2-CH3, VL3-CH1-VL4-VH4-VH3-CL-CH2-CH3, VL3-CH1-VH4-VL4-VH3-CL-CH2-CH3, VH3-CL-VL4-VH4-VL3-CH1-CH2-CH3, VH3-CL-VH4-VL4-VL3-CH1-CH2-CH3, VH3-CH1-VL4-VH4-VL3-CL-CH2-CH3, VH3-CH1-VH4-VL4-VL3-CL-CH2-CH3, VL4-CL-VL3-VH3-VH4-CH1-CH2-CH3, VL4-CL-VH3-VL3-VH4-CH1-CH2-CH3, VL4-CH1-VL3-VH3-VH4-CL-CH2-CH3, VL4-CH1-VH3-VL3-VH4-CL-CH2-CH3, VH4-CL-VL3-VH3-VL4-CH1-CH2-CH3, VH4-CL-VH3-VL3-VL4-CH1-CH2-CH3, VH4-CH1-VL3-VH3-VL4-CL-CH2-CH3, or VH4-CH1-VH3-VL3-VL4-CL-CH2-CH3.

In some aspects, an antigen binding polypeptide complex provided herein comprises a first polypeptide, and a second polypeptide; wherein the first polypeptide has a structure represented by VL1-L1-CL-L2-VL2-L3-VH2-L4-VH1-L5-CH1-CH2-CH3, VL1-L1-CL-L2-VH2-L3-VL2-L4-VH1-L5-CH1-CH2-CH3, VL1-L1-CH1-L2-VL2-L3-VH2-L4-VH1-L5-CL-CH2-CH3, VL1-L1-CH1-L2-VH2-L3-VL2-L4-VH1-L5-CL-CH2-CH3, VH1-L1-CL-L2-VL2-L3-VH2-L4-VL1-L5-CH1-CH2-CH3, VH1-L1-CL- L2-VH2-L3-VL2-L4-VL1-L5-CH1-CH2-CH3, VH1-L1-CH1-L2-VL2-L3-VH2-L4-VL1-L5-CL-CH2-CH3, VH1-L1-CH1-L2-VH2-L3-VL2-L4-VL1-L5-CL-CH2-CH3, VL2-L1-CL-L2-VL1-L3-VH1-L4-VH2-L5-CH1-CH2-CH3, VL2-L1-CL-L2-VH1-L3-VL1-L4-VH2-L5-CH1-CH2-CH3, VL2-L1-CH1-L2-VL1-L3-VH1-L4-VH2-L5-CL-CH2-CH3, VL2-L1-CH1-L2-VH1-L3-VL1-L4-VH2-L5-CL-CH2-CH3, VH2-L1-CL-L2-VL1-L3-VH1-L4-VL2-L5-CH1-CH2-CH3, VH2-L1-CL-L2-VH1-L3-VL1-L4-VL2-L5-CH1-CH2-CH3, VH2-L1-CH1-L2-VL1-L3-VH1-L4-VL2-L5-CL-CH2-CH3, or VH2-L1-CH1-L2-VH1-L3-VL1-L4-VL2-L5-CL-CH2-CH3; and wherein the second polypeptide has a structure represented by VL3-L6-CL-L7-VL4-L8-VH4-L9-VH3-L10-CH1-CH2-CH3, VL3-L6-CL-L7-VH4-L8-VL4-L9-VH3-L10-CH1-CH2-CH3, VL3-L6-CH1-L7-VL4-L8-VH4-L9-VH3-L10-CL-CH2-CH3, VL3-L6-CH1-L7-VH4-L8-VL4-L9- VH3-L10-CL-CH2-CH3, VH3-L6-CL-L7-VL4-L8-VH4-L9-VL3-L10-CH1-CH2-CH3, VH3-L6-CL-L7-VH4-L8-VL4-L9-VL3-L10-CH1-CH2-CH3, VH3-L6-CH1-L7-VL4-L8-VH4-L9-VL3-L10-CL-CH2-CH3, VH3-L6-CH1-L7-VH4-L8-VL4-L9-VL3-L10-CL-CH2-CH3, VL4-L6-CL-L7-VL3-L8-VH3-L9-VH4-L10-CH1-CH2-CH3, VL4-L6-CL-L7-VH3-L8-VL3-L9-VH4-L10-CH1-CH2-CH3, VL4-L6-CH1-L7-VL3-L8-VH3-L9-VH4-L10-CL-CH2-CH3, VL4-L6-CH1-L7-VH3-L8-VL3-L9-VH4-L10-CL-CH2-CH3, VH4-L6-CL- L7-VL3-L8-VH3-L9-VL4-L10-CH1-CH2-CH3, VH4-L6-CL-L7-VH3-L8-VL3-L9-VL4-L10-CH1- CH2-CH3, VH4-L6-CH1-L7-VL3-L8-VH3-L9-VL4-L10-CL-CH2-CH3, or VH4-L6-CH1-L7-VH3-L8-VL3-L9-VL4-L10-CL-CH2-CH3.

In some aspects, an antigen binding polypeptide complex provided herein comprises a first polypeptide, and a second polypeptide; wherein the first polypeptide has a structure represented by VL1-CL-L1-VL2-L2-VH2-L3-VH1-CH1-CH2-CH3; VL1-CL-L1-VH2-L2-VL2-L3-VH1-CH1-CH2-CH3; VL1-CH1-L1-VL2-L2-VH2-L3-VH1-CL-CH2-CH3; VL1-CH1-L1-VH2-L2-VL2-L3-VH1-CL-CH2-CH3; VH1-CL-L1-VL2-L2-VH2-L3-VL1-CH1-CH2-CH3; VH1-CL-L1-VH2-L2-VL2-L3-VL1-CH1-CH2-CH3; VH1-CH1-L1-VL2-L2-VH2-L3-VL1-CL-CH2-CH3; VH1-CH1-L1-VH2-L2-VL2-L3-VL1-CL-CH2-CH3; VL2-CL-L1-VL1-L2-VH1-L3-VH2-CH1-CH2-CH3; VL2-CL-L1-VH1-L2-VL1-L3-VH2-CH1-CH2-CH3; VL2-CH1-L1-VL1-L2-VH1-L3-VH2-CL-CH2-CH3; VL2-CH1-L1-VH1-L2-VL1-L3-VH2-CL-CH2-CH3; VH2-CL-L1-VL1-L2-VH1-L3-VL2-CH1-CH2-CH3; VH2-CL-L1-VH1-L2-VL1-L3-VL2-CH1-CH2-CH3; VH2-CH1-L1-VL1-L2-VH1-L3-VL2-CL-CH2-CH3; or VH2-CH1-L1-VH1-L2-VL1-L3-VL2-CL-CH2-CH3; and wherein the second polypeptide has a structure represented by VL3-CL-L4-VL4-L5-VH4-L6-VH3-CH1-CH2-CH3; VL3-CL-L4-VH4-L5-VL4-L6-VH3-CH1-CH2-CH3; VL3-CH1-L4-VL4-L5-VH4-L6-VH3-CL-CH2-CH3; VL3-CH1-L4-VH4-L5-VL4-L6-VH3-CL-CH2-CH3; VH3-CL-L4-VL4-L5-VH4-L6-VL3-CH1-CH2-CH3; VH3-CL-L4-VH4-L5-VL4-L6-VL3-CH1-CH2-CH3; VH3-CH1-L4-VL4-L5-VH4-L6-VL3-CL-CH2-CH3; VH3-CH1-L4-VH4-L5-VL4-L6-VL3-CL-CH2-CH3; VL4-CL-L4-VL3-L5-VH3-L6-VH4-CH1-CH2-CH3; VL4-CL-L4-VH3-L5-VL3-L6-VH4-CH1-CH2-CH3; VL4-CH1-L4-VL3-L5-VH3-L6-VH4-CL-CH2-CH3; VL4-CH1-L4-VH3-L5-VL3-L6-VH4-CL-CH2-CH3; VH4-CL-L4-VL3-L5-VH3-L6-VL4-CH1-CH2-CH3; VH4-CL-L4-VH3-L5-VL3-L6-VL4-CH1-CH2-CH3; VH4-CH1-L4-VL3-L5-VH3-L6-VL4-CL-CH2-CH3; or VH4-CH1-L4-VH3-L5-VL3-L6-VL4-CL-CH2-CH3.

In some aspects, an antigen binding polypeptide complex provided herein comprises a first polypeptide, and a second polypeptide; wherein the first polypeptide has a structure represented by VL1-VH1-VL2-CL-VH2-CH1, VL1-VH1-VL2-CH1-VH2-CL, VL1-VH1-VH2-CL-VL2-CH1, VL1-VH1-VH2-CH1-VL2-CL, VL1-VL2-VH1-CL-VH2-CH1, VL1-VL2-VH1-CH1-VH2-CL, VL1-VH2-VH1-CL-VL2-CH1, VL1-VH2-VH1-CH1-VL2-CL, VH1-VL1-VL2-CL-VH2-CH1, VH1-VL1-VL2-CH1-VH2-CL, VH1-VL1-VH2-CL-VL2-CH1, VH1-VL1-VH2-CH1-VL2-CL, VH1-VL2-VL1-CL-VH2-CH1, VH1-VL2-VL1-CH1-VH2-CL, VH1-VH2-VL1-CL-VL2-CH1, VH1-VH2-VL1-CH1-VL2-CL, VL2-VL1-VH2-CL-VH1-CH1, VL2-VL1-VH2-CH1-VH1-CL, VL2-VH1-VH2-CL-VL1-CH1, VL2-VH1-VH2-CH1-VL1-CL, VL2-VH2-VL1-CL-VH1-CH1, VL2-VH2-VL1-CH1-VH1-CL, VL2-VH2-VH1-CL-VL1-CH1, VL2-VH2-VH1-CH1-VL1-CL, VH2-VL1-VL2-CL-VH1-CH1, VH2-VL1-VL2-CH1-VH1-CL, VH2-VH1-VL2-CL-VL1-CH1, VH2-VH1-VL2-CH1-VL1-CL, VH2-VL2-VL1-CL-VH1-CH1, VH2-VL2-VL1-CH1-VH1-CL, VH2-VL2-VH1-CL-VL1-CH1, or VH2-VL2-VH1-CH1-VL1-CL; and wherein the second polypeptide has a structure represented by VL3-VH3-VL4-CL-VH4-CH1, VL3-VH3-VL4-CH1-VH4-CL, VL3-VH3-VH4-CL-VL4-CH1, VL3-VH3-VH4-CH1-VL4-CL, VL3-VL4-VH3-CL-VH4-CH1, VL3-VL4-VH3-CH1-VH4-CL, VL3-VH4-VH3-CL-VL4-CH1, VL3-VH4-VH3-CH1-VL4-CL, VH3-VL3-VL4-CL-VH4-CH1, VH3-VL3-VL4-CH1-VH4-CL, VH3-VL3-VH4-CL-VL4-CH1, VH3-VL3-VH4-CH1-VL4-CL, VH3-VL4-VL3-CL-VH4-CH1, VH3-VL4-VL3-CH1-VH4-CL, VH3-VH4-VL3-CL-VL4-CH1, VH3-VH4-VL3-CH1-VL4-CL, VL4-VL3-VH4-CL-VH3-CH1, VL4-VL3-VH4-CH1-VH3-CL, VL4-VH3-VH4-CL-VL3-CH1, VL4-VH3-VH4-CH1-VL3-CL, VL4-VH4-VL3-CL-VH3-CH1, VL4-VH4-VL3-CH1-VH3-CL, VL4-VH4-VH3-CL-VL3-CH1, VL4-VH4-VH3-CH1-VL3-CL, VH4-VL3-VL4-CL-VH3-CH1, VH4-VL3-VL4-CH1-VH3-CL, VH4-VH3-VL4-CL-VL3-CH1, VH4-VH3-VL4-CH1-VL3-CL, VH4-VL4-VL3-CL-VH3-CH1, VH4-VL4-VL3-CH1-VH3-CL, VH4-VL4-VH3-CL-VL3-CH1, or VH4-VL4-VH3-CH1-VL3-CL.

In some aspects, an antigen binding polypeptide complex provided herein comprises a first polypeptide, and a second polypeptide; wherein the first polypeptide has a structure represented by VL1-L1-VH1-L2-VL2-L3-CL-L4-VH2-L5-CH1, VL1-L1-VH1-L2-VL2-L3-CH1-L4-VH2-L5-CL, VL1-L1-VH1-L2-VH2-L3-CL-L4-VL2-L5-CH1, VL1-L1-VH1-L2-VH2-L3-CH1-L4-VL2-L5-CL, VL1-L1-VL2-L2-VH1-L3-CL-L4-VH2-L5-CH1, VL1-L1-VL2-L2-VH1-L3-CH1-L4-VH2-L5-CL, VL1-L1-VH2-L2-VH1-L3-CL-L4-VL2-L5-CH1, VL1-L1-VH2-L2-VH1-L3-CH1-L4-VL2-L5-CL, VH1-L1-VL1-L2-VL2-L3-CL-L4-VH2-L5-CH1, VH1-L1-VL1-L2-VL2-L3-CH1-L4-VH2-L5-CL, VH1-L1-VL1-L2-VH2-L3-CL-L4-VL2-L5-CH1, VH1-L1-VL1-L2-VH2-L3-CH1-L4-VL2-L5-CL, VH1-L1-VL2-L2-VL1-L3-CL-L4-VH2-L5-CH1, VH1-L1-VL2-L2-VL1-L3-CH1-L4-VH2-L5-CL, VH1-L1-VH2-L2-VL1-L3-CL-L4-VL2-L5-CH1, VH1-L1-VH2-L2-VL1-L3-CH1-L4-VL2-L5-CL, VL2-L1-VL1-L2-VH2-L3-CL-L4-VH1-L5-CH1, VL2-L1-VL1-L2-VH2-L3-CH1-L4-VH1-L5-CL, VL2-L1-VH1-L2-VH2-L3-CL-L4-VL1-L5-CH1, VL2-L1-VH1-L2-VH2-L3-CH1-L4-VL1-L5-CL, VL2-L1-VH2-L2-VL1-L3-CL-L4-VH1-L5-CH1, VL2-L1-VH2-L2-VL1-L3-CH1-L4-VH1-L5-CL, VL2-L1-VH2-L2-VH1-L3-CL-L4-VL1-L5-CH1, VL2-L1-VH2-L2-VH1-L3-CH1-L4-VL1-L5-CL, VH2-L1-VL1-L2-VL2-L3-CL-L4-VH1-L5-CH1, VH2-L1-VL1-L2-VL2-L3-CH1-L4-VH1-L5-CL, VH2-L1-VH1-L2-VL2-L3-CL-L4-VL1-L5-CH1, VH2-L1-VH1-L2-VL2-L3-CH1-L4-VL1-L5-CL, VH2-L1-VL2-L2-VL1-L3-CL-L4-VH1-L5-CH1, VH2-L1-VL2-L2-VL1-L3-CH1-L4-VH1-L5-CL, VH2-L1-VL2-L2-VH1-L3-CL-L4-VL1-L5-CH1, or VH2-L1-VL2-L2-VH1-L3-CH1-L4-VL1-L5-CL; and wherein the second polypeptide has a structure represented by VL3-L6-VH3-L7-VL4-L8-CL-L9-VH4-L10-CH1, VL3-L6-VH3-L7-VL4-L8-CH1-L9-VH4-L10-CL, VL3-L6-VH3-L7-VH4-L8-CL-L9-VL4-L10-CH1, VL3-L6-VH3-L7-VH4-L8-CH1-L9-VL4-L10-CL, VL3-L6-VL4-L7-VH3-L8-CL-L9-VH4-L10-CH1, VL3-L6-VL4-L7-VH3-L8-CH1-L9-VH4-L10-CL, VL3-L6-VH4-L7-VH3-L8-CL-L9-VL4-L10-CH1, VL3-L6-VH4-L7-VH3-L8-CH1-L9-VL4-L10-CL, VH3-L6-VL3-L7-VL4-L8-CL-L9-VH4-L10-CH1, VH3-L6-VL3-L7-VL4-L8-CH1-L9-VH4-L10-CL, VH3-L6-VL3-L7-VH4-L8-CL-L9-VL4-L10-CH1, VH3-L6-VL3-L7-VH4-L8-CH1-L9-VL4-L10-CL, VH3-L6-VL4-L7-VL3-L8-CL-L9-VH4-L10-CH1, VH3-L6-VL4-L7-VL3-L8-CH1-L9-VH4-L10-CL, VH3-L6-VH4-L7-VL3-L8-CL-L9-VL4-L10-CH1, VH3-L6-VH4-L7-VL3-L8-CH1-L9-VL4-L10-CL, VL4-L6-VL3-L7-VH4-L8-CL-L9-VH3-L10-CH1, VL4-L6-VL3-L7-VH4-L8-CH1-L9-VH3-L10-CL, VL4-L6-VH3-L7-VH4-L8-CL-L9-VL3-L10-CH1, VL4-L6-VH3-L7-VH4-L8-CH1-L9-VL3-L10-CL, VL4-L6-VH4-L7-VL3-L8-CL-L9-VH3-L10-CH1, VL4-L6-VH4-L7-VL3-L8-CH1-L9-VH3-L10-CL, VL4-L6-VH4-L7-VH3-L8-CL- L9-VL3-L10-CH1, VL4-L6-VH4-L7-VH3-L8-CH1-L9-VL3-L10-CL, VH4-L6-VL3-L7-VL4-L8-CL-L9-VH3-L10-CH1, VH4-L6-VL3-L7-VL4-L8-CH1-L9-VH3-L10-CL, VH4-L6-VH3-L7-VL4-L8-CL-L9-VL3-L10-CH1, VH4-L6-VH3-L7-VL4-L8-CH1-L9-VL3-L10-CL, VH4-L6-VL4-L7-VL3-L8-CL-L9-VH3-L10-CH1, VH4-L6-VL4-L7-VL3-L8-CH1-L9-VH3-L10-CL, VH4-L6-VL4-L7-VH3-L8-CL-L9-VL3-L10-CH1, or VH4-L6-VL4-L7-VH3-L8-CH1-L9-VL3-L10-CL.

In some aspects, an antigen binding polypeptide complex provided herein comprises a first polypeptide, and a second polypeptide; wherein the first polypeptide has a structure represented by VL1-VH1-VL2-CL-VH2-CH1-CH2-CH3, VL1-VH1-VL2-CH1-VH2-CL-CH2-CH3, VL1-VH1-VH2-CL-VL2-CH1-CH2-CH3, VL1-VH1-VH2-CH1-VL2-CL-CH2-CH3, VL1-VL2-VH1-CL-VH2-CH1-CH2-CH3, VL1-VL2-VH1-CH1-VH2-CL-CH2-CH3, VL1-VH2-VH1-CL-VL2-CH1-CH2-CH3, VL1-VH2-VH1-CH1-VL2-CL-CH2-CH3, VH1-VL1-VL2-CL-VH2-CH1-CH2-CH3, VH1-VL1-VL2-CH1-VH2-CL-CH2-CH3, VH1-VL1-VH2-CL-VL2-CH1-CH2-CH3, VH1-VL1-VH2-CH1-VL2-CL-CH2-CH3, VH1-VL2-VL1-CL-VH2-CH1-CH2-CH3, VH1-VL2-VL1-CH1-VH2-CL-CH2-CH3, VH1-VH2-VL1-CL-VL2-CH1-CH2-CH3, VH1-VH2-VL1-CH1-VL2-CL-CH2-CH3, VL2-VL1-VH2-CL-VH1-CH1-CH2-CH3, VL2-VL1-VH2-CH1-VH1-CL-CH2-CH3, VL2-VH1-VH2-CL-VL1-CH1-CH2-CH3, VL2-VH1-VH2-CH1-VL1-CL-CH2-CH3, VL2-VH2-VL1-CL-VH1-CH1-CH2-CH3, VL2-VH2-VL1-CH1-VH1-CL-CH2-CH3, VL2-VH2-VH1-CL-VL1-CH1-CH2-CH3, VL2-VH2-VH1-CH1-VL1-CL-CH2-CH3, VH2-VL1-VL2-CL-VH1-CH1-CH2-CH3, VH2-VL1-VL2-CH1-VH1-CL-CH2-CH3, VH2-VH1-VL2-CL-VL1-CH1-CH2-CH3, VH2-VH1-VL2-CH1-VL1-CL-CH2-CH3, VH2-VL2-VL1-CL-VH1-CH1-CH2-CH3, VH2-VL2-VL1-CH1-VH1-CL-CH2-CH3, VH2-VL2-VH1-CL-VL1-CH1-CH2-CH3, or VH2-VL2-VH1-CH1-VL1-CL-CH2-CH3; and wherein the second polypeptide has a structure represented by VL3-VH3-VL4-CL-VH4-CH1-CH2-CH3, VL3-VH3-VL4-CH1-VH4-CL-CH2-CH3, VL3-VH3-VH4-CL-VL4-CH1-CH2-CH3, VL3-VH3-VH4-CH1-VL4-CL-CH2-CH3, VL3-VL4-VH3-CL-VH4-CH1-CH2-CH3, VL3-VL4-VH3-CH1-VH4-CL-CH2-CH3, VL3-VH4-VH3-CL-VL4-CH1-CH2-CH3, VL3-VH4-VH3-CH1-VL4-CL-CH2-CH3, VH3-VL3-VL4-CL-VH4-CH1-CH2-CH3, VH3-VL3-VL4-CH1-VH4-CL-CH2-CH3, VH3-VL3-VH4-CL-VL4-CH1-CH2-CH3, VH3-VL3-VH4-CH1-VL4-CL-CH2-CH3, VH3-VL4-VL3-CL-VH4-CH1-CH2-CH3, VH3-VL4-VL3-CH1-VH4-CL-CH2-CH3, VH3-VH4-VL3-CL-VL4-CH1-CH2-CH3, VH3-VH4-VL3-CH1-VL4-CL-CH2-CH3, VL4-VL3-VH4-CL-VH3-CH1-CH2-CH3, VL4-VL3-VH4-CH1-VH3-CL-CH2-CH3, VL4-VH3-VH4-CL-VL3-CH1-CH2-CH3, VL4-VH3-VH4-CH1-VL3-CL-CH2-CH3, VL4-VH4-VL3-CL-VH3-CH1-CH2-CH3, VL4-VH4-VL3-CH1-VH3-CL-CH2-CH3, VL4-VH4-VH3-CL-VL3-CH1-CH2-CH3, VL4-VH4-VH3-CH1-VL3-CL-CH2-CH3, VH4-VL3-VL4-CL-VH3-CH1-CH2-CH3, VH4-VL3-VL4-CH1-VH3-CL-CH2-CH3, VH4-VH3-VL4-CL-VL3-CH1-CH2-CH3, VH4-VH3-VL4-CH1-VL3-CL-CH2-CH3, VH4-VL4-VL3-CL-VH3-CH1-CH2-CH3, VH4-VL4-VL3-CH1-VH3-CL-CH2-CH3, VH4-VL4-VH3-CL-VL3-CH1-CH2-CH3, or VH4-VL4-VH3-CH1-VL3-CL-CH2-CH3.

In some aspects, an antigen binding polypeptide complex provided herein comprises a first polypeptide, and a second polypeptide; wherein the first polypeptide has a structure represented by VL1-L1-VH1-L2-VL2-L3-CL-L4-VH2-L5-CH1-CH2-CH3, VL1-L1-VH1-L2-VL2-L3-CH1-L4-VH2- L5-CL-CH2-CH3, VL1-L1-VH1-L2-VH2-L3-CL-L4-VL2-L5-CH1-CH2-CH3, VL1-L1-VH1-L2-VH2-L3-CH1-L4-VL2-L5-CL-CH2-CH3, VL1-L1-VL2-L2-VH1-L3-CL-L4-VH2-L5-CH1-CH2-CH3, VL1-L1-VL2-L2-VH1-L3-CH1-L4-VH2-L5-CL-CH2-CH3, VL1-L1-VH2-L2-VH1-L3-CL-L4-VL2-L5-CH1-CH2-CH3, VL1-L1-VH2-L2-VH1-L3-CH1-L4-VL2-L5-CL-CH2-CH3, VH1-L1-VL1-L2-VL2-L3-CL-L4-VH2-L5-CH1-CH2-CH3, VH1-L1-VL1-L2-VL2-L3-CH1-L4-VH2-L5-CL-CH2-CH3, VH1-L1-VL1-L2-VH2-L3-CL-L4-VL2-L5-CH1-CH2-CH3, VH1-L1-VL1-L2-VH2-L3-CH1-L4-VL2-L5-CL-CH2-CH3, VH1-L1-VL2-L2-VL1-L3-CL-L4-VH2-L5-CH1-CH2-CH3, VH1-L1-VL2-L2-VL1-L3-CH1-L4-VH2-L5- CL-CH2-CH3, VH1-L1-VH2-L2-VL1-L3-CL-L4-VL2-L5-CH1-CH2-CH3, VH1-L1-VH2-L2-VL1-L3-CH1-L4-VL2-L5-CL-CH2-CH3, VL2-L1-VL1-L2-VH2-L3-CL-L4-VH1-L5-CH1-CH2-CH3, VL2-L1-VL1-L2-VH2-L3-CH1-L4-VH1-L5-CL-CH2-CH3, VL2-L1-VH1-L2-VH2-L3-CL-L4-VL1-L5-CH1-CH2-CH3, VL2-L1-VH1-L2-VH2-L3-CH1-L4-VL1-L5-CL-CH2-CH3, VL2-L1-VH2-L2-VL1-L3-CL-L4-VH1-L5-CH1-CH2-CH3, VL2-L1-VH2-L2-VL1-L3-CH1-L4-VH1-L5-CL-CH2-CH3, VL2-L1-VH2-L2-VH1-L3-CL-L4-VL1-L5-CH1-CH2-CH3, VL2-L1-VH2-L2-VH1-L3-CH1-L4-VL1-L5-CL-CH2-CH3, VH2-L1-VL1-L2-VL2-L3-CL-L4-VH1-L5-CH1-CH2-CH3, VH2-L1-VL1-L2-VL2-L3-CH1-L4-VH1-L5- CL-CH2-CH3, VH2-L1-VH1-L2-VL2-L3-CL-L4-VL1-L5-CH1-CH2-CH3, VH2-L1-VH1-L2-VL2-L3-CH1-L4-VL1-L5-CL-CH2-CH3, VH2-L1-VL2-L2-VL1-L3-CL-L4-VH1-L5-CH1-CH2-CH3, VH2-L1-VL2-L2-VL1-L3-CH1-L4-VH1-L5-CL-CH2-CH3, VH2-L1-VL2-L2-VH1-L3-CL-L4-VL1-L5-CH1-CH2-CH3, or VH2-L1-VL2-L2-VH1-L3-CH1-L4-VL1-L5-CL-CH2-CH3; and wherein the second polypeptide has a structure represented by VL3-L6-VH3-L7-VL4-L8-CL-L9-VH4-L10-CH1-CH2-CH3, VL3-L6-VH3-L7-VL4-L8-CH1-L9-VH4-L10-CL-CH2-CH3, VL3-L6-VH3-L7-VH4-L8-CL-L9-VL4-L10-CH1-CH2-CH3, VL3-L6-VH3-L7-VH4-L8-CH1-L9-VL4-L10-CL-CH2-CH3, VL3-L6-VL4-L7-VH3-L8-CL-L9-VH4-L10-CH1-CH2-CH3, VL3-L6-VL4-L7-VH3-L8-CH1-L9-VH4-L10-CL-CH2-CH3, VL3-L6-VH4-L7-VH3-L8-CL-L9-VL4-L10-CH1-CH2-CH3, VL3-L6-VH4-L7-VH3-L8-CH1-L9-VL4-L10-CL-CH2-CH3, VH3- L6-VL3-L7-VL4-L8-CL-L9-VH4-L10-CH1-CH2-CH3, VH3-L6-VL3-L7-VL4-L8-CH1-L9-VH4-L10-CL-CH2-CH3, VH3-L6-VL3-L7-VH4-L8-CL-L9-VL4-L10-CH1-CH2-CH3, VH3-L6-VL3-L7-VH4-L8- CH1-L9-VL4-L10-CL-CH2-CH3, VH3-L6-VL4-L7-VL3-L8-CL-L9-VH4-L10-CH1-CH2-CH3, VH3-L6- VL4-L7-VL3-L8-CH1-L9-VH4-L10-CL-CH2-CH3, VH3-L6-VH4-L7-VL3-L8-CL-L9-VL4-L10-CH1-CH2-CH3, VH3-L6-VH4-L7-VL3-L8-CH1-L9-VL4-L10-CL-CH2-CH3, VL4-L6-VL3-L7-VH4-L8-CL-L9-VH3-L10-CH1-CH2-CH3, VL4-L6-VL3-L7-VH4-L8-CH1-L9-VH3-L10-CL-CH2-CH3, VL4-L6-VH3-L7-VH4-L8-CL-L9-VL3-L10-CH1-CH2-CH3, VL4-L6-VH3-L7-VH4-L8-CH1-L9-VL3-L10-CL-CH2-CH3, VL4-L6-VH4-L7-VL3-L8-CL-L9-VH3-L10-CH1-CH2-CH3, VL4-L6-VH4-L7-VL3-L8-CH1-L9-VH3-L10-CL-CH2-CH3, VL4-L6-VH4-L7-VH3-L8-CL-L9-VL3-L10-CH1-CH2-CH3, VL4-L6-VH4- L7-VH3-L8-CH1-L9-VL3-L10-CL-CH2-CH3, VH4-L6-VL3-L7-VL4-L8-CL-L9-VH3-L10-CH1-CH2-CH3, VH4-L6-VL3-L7-VL4-L8-CH1-L9-VH3-L10-CL-CH2-CH3, VH4-L6-VH3-L7-VL4-L8-CL-L9-VL3-L10-CH1-CH2-CH3, VH4-L6-VH3-L7-VL4-L8-CH1-L9-VL3-L10-CL-CH2-CH3, VH4-L6-VL4-L7-VL3-L8-CL-L9-VH3-L10-CH1-CH2-CH3, VH4-L6-VL4-L7-VL3-L8-CH1-L9-VH3-L10-CL-CH2-CH3, VH4- L6-VL4-L7-VH3-L8-CL-L9-VL3-L10-CH1-CH2-CH3, or VH4-L6-VL4-L7-VH3-L8-CH1-L9-VL3-L10-CL-CH2-CH3.

In some aspects, an antigen binding polypeptide complex provided herein comprises a first polypeptide, and a second polypeptide; wherein the first polypeptide has a structure represented by VL1-CL-VH1-CH1-VL2-VH2, VL1-CL-VH1-CH1-VH2-VL2, VL1-CL-VL2-CH1-VH1-VH2, VL1-CL-VH2-CH1-VH1-VL2, VL1-CH1-VH1-CL-VL2-VH2, VL1-CH1-VH1-CL-VH2-VL2, VL1-CH1-VL2-CL-VH1-VH2, VL1-CH1-VH2-CL-VH1-VL2, VH1-CL-VL1-CH1-VL2-VH2, VH1-CL-VL1-CH1-VH2-VL2, VH1-CL-VL2-CH1-VL1-VH2, VH1-CL-VH2-CH1-VL1-VL2, VH1-CH1-VL1-CL-VL2-VH2, VH1-CH1-VL1-CL-VH2-VL2, VH1-CH1-VL2-CL-VL1-VH2, VH1-CH1-VH2-CL-VL1-VL2, VL2-CL-VL1-CH1-VH2-VH1, VL2-CL-VH1-CH1-VH2-VL1, VL2-CL-VH2-CH1-VL1-VH1, VL2-CL-VH2-CH1-VH1-VL1, VL2-CH1-VL1-CL-VH2-VH1, VL2-CH1-VH1-CL-VH2-VL1, VL2-CH1-VH2-CL-VL1-VH1, VL2-CH1-VH2-CL-VH1-VL1, VH2-CL-VL1-CH1-VL2-VH1, VH2-CL-VH1-CH1-VL2-VL1, VH2-CL-VL2-CH1-VL1-VH1, VH2-CL-VL2-CH1-VH1-VL1, VH2-CH1-VL1-CL-VL2-VH1, VH2-CH1-VH1-CL-VL2-VL1, VH2-CH1-VL2-CL-VL1-VH1, or VH2-CH1-VL2-CL-VH1-VL1; and wherein the second polypeptide has a structure represented by VL3-CL-VH3-CH1-VL4-VH4, VL3-CL-VH3-CH1-VH4-VL4, VL3-CL-VL4-CH1-VH3-VH4, VL3-CL-VH4-CH1-VH3-VL4, VL3-CH1-VH3-CL-VL4-VH4, VL3-CH1-VH3-CL-VH4-VL4, VL3-CH1-VL4-CL-VH3-VH4, VL3-CH1-VH4-CL-VH3-VL4, VH3-CL-VL3-CH1-VL4-VH4, VH3-CL-VL3-CH1-VH4-VL4, VH3-CL-VL4-CH1-VL3-VH4, VH3-CL-VH4-CH1-VL3-VL4, VH3-CH1-VL3-CL-VL4-VH4, VH3-CH1-VL3-CL-VH4-VL4, VH3-CH1-VL4-CL-VL3-VH4, VH3-CH1-VH4-CL-VL3-VL4, VL4-CL-VL3-CH1-VH4-VH3, VL4-CL-VH3-CH1-VH4-VL3, VL4-CL-VH4-CH1-VL3-VH3, VL4-CL-VH4-CH1-VH3-VL3, VL4-CH1-VL3-CL-VH4-VH3, VL4-CH1-VH3-CL-VH4-VL3, VL4-CH1-VH4-CL-VL3-VH3, VL4-CH1-VH4-CL-VH3-VL3, VH4-CL-VL3-CH1-VL4-VH3, VH4-CL-VH3-CH1-VL4-VL3, VH4-CL-VL4-CH1-VL3-VH3, VH4-CL-VL4-CH1-VH3-VL3, VH4-CH1-VL3-CL-VL4-VH3, VH4-CH1-VH3-CL-VL4-VL3, VH4-CH1-VL4-CL-VL3-VH3, or VH4-CH1-VL4-CL-VH3-VL3.

In some aspects, an antigen binding polypeptide complex provided herein comprises a first polypeptide, and a second polypeptide; wherein the first polypeptide has a structure represented by VL1-L1-CL-L2-VH1-L3-CH1-L4-VL2-L5-VH2, VL1-L1-CL-L2-VH1-L3-CH1-L4-VH2-L5-VL2, VL1-L1-CL-L2-VL2-L3-CH1-L4-VH1-L5-VH2, VL1-L1-CL-L2-VH2-L3-CH1-L4-VH1-L5-VL2, VL1-L1-CH1-L2-VH1-L3-CL-L4-VL2-L5-VH2, VL1-L1-CH1-L2-VH1-L3-CL-L4-VH2-L5-VL2, VL1-L1-CH1-L2-VL2-L3-CL-L4-VH1-L5-VH2, VL1-L1-CH1-L2-VH2-L3-CL-L4-VH1-L5-VL2, VH1-L1-CL-L2-VL1-L3-CH1-L4-VL2-L5-VH2, VH1-L1-CL-L2-VL1-L3-CH1-L4-VH2-L5-VL2, VH1-L1-CL-L2-VL2-L3-CH1-L4-VL1-L5-VH2, VH1-L1-CL-L2-VH2-L3-CH1-L4-VL1-L5-VL2, VH1-L1-CH1-L2-VL1-L3- CL-L4-VL2-L5-VH2, VH1-L1-CH1-L2-VL1-L3-CL-L4-VH2-L5-VL2, VH1-L1-CH1-L2-VL2-L3-CL-L4-VL1-L5-VH2, VH1-L1-CH1-L2-VH2-L3-CL-L4-VL1-L5-VL2, VL2-L1-CL-L2-VL1-L3-CH1-L4-VH2-L5-VH1, VL2-L1-CL-L2-VH1-L3-CH1-L4-VH2-L5-VL1, VL2-L1-CL-L2-VH2-L3-CH1-L4-VL1-L5-VH1, VL2-L1-CL-L2-VH2-L3-CH1-L4-VH1-L5-VL1, VL2-L1-CH1-L2-VL1-L3-CL-L4-VH2-L5-VH1, VL2-L1-CH1-L2-VH1-L3-CL-L4-VH2-L5-VL1, VL2-L1-CH1-L2-VH2-L3-CL-L4-VL1-L5-VH1, VL2-L1-CH1-L2-VH2-L3-CL-L4-VH1-L5-VL1, VH2-L1-CL-L2-VL1-L3-CH1-L4-VL2-L5-VH1, VH2-L1-CL-L2-VH1-L3-CH1-L4-VL2-L5-VL1, VH2-L1-CL-L2-VL2-L3-CH1-L4-VL1-L5-VH1, VH2-L1-CL- L2-VL2-L3-CH1-L4-VH1-L5-VL1, VH2-L1-CH1-L2-VL1-L3-CL-L4-VL2-L5-VH1, VH2-L1-CH1-L2-VH1-L3-CL-L4-VL2-L5-VL1, VH2-L1-CH1-L2-VL2-L3-CL-L4-VL1-L5-VH1, or VH2-L1-CH1-L2-VL2-L3-CL-L4-VH1-L5-VL1; and wherein the second polypeptide has a structure represented by VL3-L6-CL-L7-VH3-L8-CH1-L9-VL4-L10-VH4, VL3-L6-CL-L7-VH3-L8-CH1-L9-VH4-L10-VL4, VL3-L6-CL-L7-VL4-L8-CH1-L9-VH3-L10-VH4, VL3-L6-CL-L7-VH4-L8-CH1-L9-VH3-L10-VL4, VL3-L6-CH1-L7-VH3-L8-CL-L9-VL4-L10-VH4, VL3-L6-CH1-L7-VH3-L8-CL-L9-VH4-L10-VL4, VL3-L6-CH1-L7-VL4-L8-CL-L9-VH3-L10-VH4, VL3-L6-CH1-L7-VH4-L8-CL-L9-VH3-L10-VL4, VH3-L6-CL-L7- VL3-L8-CH1-L9-VL4-L10-VH4, VH3-L6-CL-L7-VL3-L8-CH1-L9-VH4-L10-VL4, VH3-L6-CL-L7-VL4-L8-CH1-L9-VL3-L10-VH4, VH3-L6-CL-L7-VH4-L8-CH1-L9-VL3-L10-VL4, VH3-L6-CH1-L7-VL3-L8-CL-L9-VL4-L10-VH4, VH3-L6-CH1-L7-VL3-L8-CL-L9-VH4-L10-VL4, VH3-L6-CH1-L7-VL4-L8-CL-L9-VL3-L10-VH4, VH3-L6-CH1-L7-VH4-L8-CL-L9-VL3-L10-VL4, VL4-L6-CL-L7-VL3-L8-CH1- L9-VH4-L10-VH3, VL4-L6-CL-L7-VH3-L8-CH1-L9-VH4-L10-VL3, VL4-L6-CL-L7-VH4-L8-CH1-L9-VL3-L10-VH3, VL4-L6-CL-L7-VH4-L8-CH1-L9-VH3-L10-VL3, VL4-L6-CH1-L7-VL3-L8-CL-L9-VH4-L10-VH3, VL4-L6-CH1-L7-VH3-L8-CL-L9-VH4-L10-VL3, VL4-L6-CH1-L7-VH4-L8-CL-L9-VL3-L10-VH3, VL4-L6-CH1-L7-VH4-L8-CL-L9-VH3-L10-VL3, VH4-L6-CL-L7-VL3-L8-CH1-L9-VL4-L10-VH3, VH4-L6-CL-L7-VH3-L8-CH1-L9-VL4-L10-VL3, VH4-L6-CL-L7-VL4-L8-CH1-L9-VL3-L10-VH3, VH4-L6-CL-L7-VL4-L8-CH1-L9-VH3-L10-VL3, VH4-L6-CH1-L7-VL3-L8-CL-L9-VL4-L10-VH3, VH4-L6-CH1-L7-VH3-L8-CL-L9-VL4-L10-VL3, VH4-L6-CH1-L7-VL4-L8-CL-L9-VL3-L10-VH3, or VH4-L6-CH1-L7-VL4-L8-CL-L9-VH3-L10-VL3.

In some aspects, an antigen binding polypeptide complex provided herein comprises a first polypeptide, and a second polypeptide; wherein the first polypeptide has a structure represented by VL1-CL-VH1-CH1-VL2-VH2-CH2-CH3, VL1-CL-VH1-CH1-VH2-VL2-CH2-CH3, VL1-CL-VL2-CH1-VH1-VH2-CH2-CH3, VL1-CL-VH2-CH1-VH1-VL2-CH2-CH3, VL1-CH1-VH1-CL-VL2-VH2-CH2-CH3, VL1-CH1-VH1-CL-VH2-VL2-CH2-CH3, VL1-CH1-VL2-CL-VH1-VH2-CH2-CH3, VL1-CH1-VH2-CL-VH1-VL2-CH2-CH3, VH1-CL-VL1-CH1-VL2-VH2-CH2-CH3, VH1-CL-VL1-CH1-VH2-VL2-CH2-CH3, VH1-CL-VL2-CH1-VL1-VH2-CH2-CH3, VH1-CL-VH2-CH1-VL1-VL2-CH2-CH3, VH1-CH1-VL1-CL-VL2-VH2-CH2-CH3, VH1-CH1-VL1-CL-VH2-VL2-CH2-CH3, VH1-CH1-VL2-CL-VL1-VH2-CH2-CH3, VH1-CH1-VH2-CL-VL1-VL2-CH2-CH3, VL2-CL-VL1-CH1-VH2-VH1-CH2-CH3, VL2-CL-VH1-CH1-VH2-VL1-CH2-CH3, VL2-CL-VH2-CH1-VL1-VH1-CH2-CH3, VL2-CL-VH2-CH1-VH1-VL1-CH2-CH3, VL2-CH1-VL1-CL-VH2-VH1-CH2-CH3, VL2-CH1-VH1-CL-VH2-VL1-CH2-CH3, VL2-CH1-VH2-CL-VL1-VH1-CH2-CH3, VL2-CH1-VH2-CL-VH1-VL1-CH2-CH3, VH2-CL-VL1-CH1-VL2-VH1-CH2-CH3, VH2-CL-VH1-CH1-VL2-VL1-CH2-CH3, VH2-CL-VL2-CH1-VL1-VH1-CH2-CH3, VH2-CL-VL2-CH1-VH1-VL1-CH2-CH3, VH2-CH1-VL1-CL-VL2-VH1-CH2-CH3, VH2-CH1-VH1-CL-VL2-VL1-CH2-CH3, VH2-CH1-VL2-CL-VL1-VH1-CH2-CH3, or VH2-CH1-VL2-CL-VH1-VL1-CH2-CH3; and wherein the second polypeptide has a structure represented by VL3-CL-VH3-CH1-VL4-VH4-CH2-CH3, VL3-CL-VH3-CH1-VH4-VL4-CH2-CH3, VL3-CL-VL4-CH1-VH3-VH4-CH2-CH3, VL3-CL-VH4-CH1-VH3-VL4-CH2-CH3, VL3-CH1-VH3-CL-VL4-VH4-CH2-CH3, VL3-CH1-VH3-CL-VH4-VL4-CH2-CH3, VL3-CH1-VL4-CL-VH3-VH4-CH2-CH3, VL3-CH1-VH4-CL-VH3-VL4-CH2-CH3, VH3-CL-VL3-CH1-VL4-VH4-CH2-CH3, VH3-CL-VL3-CH1-VH4-VL4-CH2-CH3, VH3-CL-VL4-CH1-VL3-VH4-CH2-CH3, VH3-CL-VH4-CH1-VL3-VL4-CH2-CH3, VH3-CH1-VL3-CL-VL4-VH4-CH2-CH3, VH3-CH1-VL3-CL-VH4-VL4-CH2-CH3, VH3-CH1-VL4-CL-VL3-VH4-CH2-CH3, VH3-CH1-VH4-CL-VL3-VL4-CH2-CH3, VL4-CL-VL3-CH1-VH4-VH3-CH2-CH3, VL4-CL-VH3-CH1-VH4-VL3-CH2-CH3, VL4-CL-VH4-CH1-VL3-VH3-CH2-CH3, VL4-CL-VH4-CH1-VH3-VL3-CH2-CH3, VL4-CH1-VL3-CL-VH4-VH3-CH2-CH3, VL4-CH1-VH3-CL-VH4-VL3-CH2-CH3, VL4-CH1-VH4-CL-VL3-VH3-CH2-CH3, VL4-CH1-VH4-CL-VH3-VL3-CH2-CH3, VH4-CL-VL3-CH1-VL4-VH3-CH2-CH3, VH4-CL-VH3-CH1-VL4-VL3-CH2-CH3, VH4-CL-VL4-CH1-VL3-VH3-CH2-CH3, VH4-CL-VL4-CH1-VH3-VL3-CH2-CH3, VH4-CH1-VL3-CL-VL4-VH3-CH2-CH3, VH4-CH1-VH3-CL-VL4-VL3-CH2-CH3, VH4-CH1-VL4-CL-VL3-VH3-CH2-CH3, or VH4-CH1-VL4-CL-VH3-VL3-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-L4-VL2-L5-VH2-CH2-CH3, VL1-L1-CL-L2-VH1-L3-CH1-L4-VH2-L5-VL2-CH2-CH3, VL1-L1-CL-L2-VL2-L3-CH1-L4-VH1-L5-VH2-CH2-CH3, VL1-L1-CL-L2-VH2-L3-CH1-L4-VH1-L5-VL2-CH2-CH3, VL1-L1-CH1-L2-VH1-L3-CL-L4-VL2-L5-VH2-CH2-CH3, VL1-L1-CH1-L2-VH1-L3-CL-L4-VH2-L5-VL2-CH2-CH3, VL1-L1-CH1-L2-VL2-L3-CL-L4-VH1-L5-VH2-CH2-CH3, VL1-L1-CH1-L2-VH2-L3-CL-L4-VH1-L5-VL2-CH2-CH3, VH1-L1-CL-L2-VL1-L3-CH1-L4-VL2-L5-VH2-CH2-CH3, VH1-L1-CL-L2-VL1-L3-CH1-L4-VH2-L5-VL2-CH2-CH3, VH1-L1-CL-L2-VL2-L3-CH1-L4-VL1-L5-VH2-CH2-CH3, VH1-L1-CL-L2-VH2-L3-CH1-L4-VL1-L5-VL2-CH2-CH3, VH1-L1-CH1-L2-VL1-L3-CL-L4-VL2-L5-VH2-CH2-CH3, VH1-L1-CH1-L2-VL1-L3-CL-L4-VH2-L5-VL2-CH2-CH3, VH1-L1-CH1-L2-VL2-L3-CL-L4-VL1-L5-VH2-CH2-CH3, VH1-L1-CH1-L2-VH2-L3-CL-L4-VL1-L5-VL2-CH2-CH3, VL2-L1-CL-L2-VL1-L3-CH1-L4-VH2-L5-VH1-CH2-CH3, VL2-L1-CL-L2-VH1-L3-CH1-L4-VH2-L5-VL1-CH2-CH3, VL2-L1-CL-L2-VH2-L3-CH1-L4-VL1-L5-VH1-CH2-CH3, VL2-L1-CL-L2-VH2-L3-CH1-L4-VH1-L5-VL1-CH2-CH3, VL2-L1-CH1-L2-VL1-L3-CL-L4-VH2-L5-VH1-CH2-CH3, VL2-L1-CH1-L2-VH1-L3-CL-L4-VH2-L5-VL1-CH2-CH3, VL2-L1-CH1-L2-VH2-L3-CL-L4-VL1-L5-VH1-CH2-CH3, VL2-L1-CH1-L2-VH2-L3-CL-L4-VH1-L5-VL1-CH2-CH3, VH2-L1-CL-L2-VL1-L3-CH1-L4-VL2-L5-VH1-CH2-CH3, VH2-L1-CL-L2-VH1-L3-CH1-L4-VL2-L5-VL1-CH2-CH3, VH2-L1-CL-L2-VL2-L3-CH1-L4-VL1-L5-VH1-CH2-CH3, VH2-L1-CL-L2-VL2-L3-CH1-L4-VH1-L5-VL1-CH2-CH3, VH2-L1-CH1-L2-VL1-L3-CL-L4-VL2-L5-VH1-CH2-CH3, VH2-L1-CH1-L2-VH1-L3-CL-L4-VL2-L5-VL1-CH2-CH3, VH2-L1-CH1-L2-VL2-L3-CL-L4-VL1-L5-VH1-CH2-CH3, or VH2-L1-CH1-L2-VL2-L3-CL-L4-VH1-L5-VL1-CH2-CH3; and wherein the second polypeptide has a structure represented by VL3-L6-CL-L7-VH3-L8-CH1-L9-VL4-L10-VH4-CH2-CH3, VL3-L6-CL-L7-VH3-L8-CH1-L9-VH4-L10-VL4-CH2-CH3, VL3-L6-CL-L7-VL4-L8-CH1-L9-VH3-L10-VH4-CH2-CH3, VL3-L6-CL-L7- VH4-L8-CH1-L9-VH3-L10-VL4-CH2-CH3, VL3-L6-CH1-L7-VH3-L8-CL-L9-VL4-L10-VH4-CH2-CH3, VL3-L6-CH1-L7-VH3-L8-CL-L9-VH4-L10-VL4-CH2-CH3, VL3-L6-CH1-L7-VL4-L8-CL-L9-VH3-L10-VH4-CH2-CH3, VL3-L6-CH1-L7-VH4-L8-CL-L9-VH3-L10-VL4-CH2-CH3, VH3-L6-CL-L7-VL3-L8-CH1-L9-VL4-L10-VH4-CH2-CH3, VH3-L6-CL-L7-VL3-L8-CH1-L9-VH4-L10-VL4-CH2-CH3, VH3-L6-CL-L7-VL4-L8-CH1-L9-VL3-L10-VH4-CH2-CH3, VH3-L6-CL-L7-VH4-L8-CH1-L9-VL3-L10-VL4- CH2-CH3, VH3-L6-CH1-L7-VL3-L8-CL-L9-VL4-L10-VH4-CH2-CH3, VH3-L6-CH1-L7-VL3-L8-CL-L9-VH4-L10-VL4-CH2-CH3, VH3-L6-CH1-L7-VL4-L8-CL-L9-VL3-L10-VH4-CH2-CH3, VH3-L6-CH1-L7-VH4-L8-CL-L9-VL3-L10-VL4-CH2-CH3, VL4-L6-CL-L7-VL3-L8-CH1-L9-VH4-L10-VH3-CH2-CH3, VL4-L6-CL-L7-VH3-L8-CH1-L9-VH4-L10-VL3-CH2-CH3, VL4-L6-CL-L7-VH4-L8-CH1-L9-VL3-L10-VH3-CH2-CH3, VL4-L6-CL-L7-VH4-L8-CH1-L9-VH3-L10-VL3-CH2-CH3, VL4-L6-CH1-L7-VL3-L8-CL-L9-VH4-L10-VH3-CH2-CH3, VL4-L6-CH1-L7-VH3-L8-CL-L9-VH4-L10-VL3-CH2-CH3, VL4-L6-CH1-L7-VH4-L8-CL-L9-VL3-L10-VH3-CH2-CH3, VL4-L6-CH1-L7-VH4-L8-CL-L9-VH3-L10-VL3-CH2-CH3, VH4-L6-CL-L7-VL3-L8-CH1-L9-VL4-L10-VH3-CH2-CH3, VH4-L6-CL-L7-VH3-L8-CH1-L9-VL4-L10-VL3-CH2-CH3, VH4-L6-CL-L7-VL4-L8-CH1-L9-VL3-L10-VH3-CH2-CH3, VH4-L6-CL-L7-VL4-L8-CH1-L9-VH3-L10-VL3-CH2-CH3, VH4-L6-CH1-L7-VL3-L8-CL-L9-VL4-L10-VH3-CH2-CH3, VH4-L6-CH1-L7-VH3-L8-CL-L9-VL4-L10-VL3-CH2-CH3, VH4-L6-CH1-L7-VL4-L8-CL-L9-VL3-L10-VH3-CH2-CH3, or VH4-L6-CH1-L7-VL4-L8-CL-L9-VH3-L10-VL3-CH2-CH3.

In some aspects, an antigen binding polypeptide complex provided herein comprises a first polypeptide, and a second polypeptide; wherein the first polypeptide has a structure represented by VL1-VL2-VH2-VH1-CL-CH1, VL1-VL2-VH2-VH1-CH1-CL, VL1-VH2-VL2-VH1-CL-CH1, VL1-VH2-VL2-VH1-CH1-CL, VH1-VL2-VH2-VL1-CL-CH1, VH1-VL2-VH2-VL1-CH1-CL, VH1-VH2-VL2-VL1-CL-CH1, VH1-VH2-VL2-VL1-CH1-CL, VL2-VL1-VH1-VH2-CL-CH1, VL2-VL1-VH1-VH2-CH1-CL, VL2-VH1-VL1-VH2-CL-CH1, VL2-VH1-VL1-VH2-CH1-CL, VH2-VL1-VH1-VL2-CL-CH1, VH2-VL1-VH1-VL2-CH1-CL, VH2-VH1-VL1-VL2-CL-CH1, or VH2-VH1-VL1-VL2-CH1-CL; and wherein the second polypeptide has a structure represented by VL3-VL4-VH4-VH3-CL-CH1, VL3-VL4-VH4-VH3-CH1-CL, VL3-VH4-VL4-VH3-CL-CH1, VL3-VH4-VL4-VH3-CH1-CL, VH3-VL4-VH4-VL3-CL-CH1, VH3-VL4-VH4-VL3-CH1-CL, VH3-VH4-VL4-VL3-CL-CH1, VH3-VH4-VL4-VL3-CH1-CL, VL4-VL3-VH3-VH4-CL-CH1, VL4-VL3-VH3-VH4-CH1-CL, VL4-VH3-VL3-VH4-CL-CH1, VL4-VH3-VL3-VH4-CH1-CL, VH4-VL3-VH3-VL4-CL-CH1, VH4-VL3-VH3-VL4-CH1-CL, VH4-VH3-VL3-VL4-CL-CH1, or VH4-VH3-VL3-VL4-CH1-CL.

In some aspects, an antigen binding polypeptide complex provided herein comprises a first polypeptide, and a second polypeptide; wherein the first polypeptide has a structure represented by VL1-L1-VL2-L2-VH2-L3-VH1-L4-CL-L5-CH1, VL1-L1-VL2-L2-VH2-L3-VH1-L4-CH1-L5-CL, VL1-L1-VH2-L2-VL2-L3-VH1-L4-CL-L5-CH1, VL1-L1-VH2-L2-VL2-L3-VH1-L4-CH1-L5-CL, VH1-L1-VL2-L2-VH2-L3-VL1-L4-CL-L5-CH1, VH1-L1-VL2-L2-VH2-L3-VL1-L4-CH1-L5-CL, VH1-L1-VH2-L2-VL2-L3-VL1-L4-CL-L5-CH1, VH1-L1-VH2-L2-VL2-L3-VL1-L4-CH1-L5-CL, VL2-L1-VL1-L2-VH1-L3-VH2-L4-CL-L5-CH1, VL2-L1-VL1-L2-VH1-L3-VH2-L4-CH1-L5-CL, VL2-L1-VH1-L2-VL1-L3-VH2-L4-CL-L5-CH1, VL2-L1-VH1-L2-VL1-L3-VH2-L4-CH1-L5-CL, VH2-L1-VL1-L2-VH1-L3-VL2-L4-CL-L5-CH1, VH2-L1-VL1-L2-VH1-L3-VL2-L4-CH1-L5-CL, VH2-L1-VH1-L2-VL1-L3-VL2-L4-CL-L5-CH1, or VH2-L1-VH1-L2-VL1-L3-VL2-L4-CH1-L5-CL; and wherein the second polypeptide has a structure represented by VL3-L6-VL4-L7-VH4-L8-VH3-L9-CL-L10-CH1, VL3-L6-VL4-L7-VH4-L8-VH3-L9-CH1-L10-CL, VL3-L6-VH4-L7-VL4-L8-VH3-L9-CL-L10-CH1, VL3-L6-VH4-L7-VL4-L8-VH3-L9-CH1-L10-CL, VH3-L6-VL4-L7-VH4-L8-VL3-L9-CL-L10-CH1, VH3-L6-VL4-L7-VH4-L8-VL3-L9-CH1-L10-CL, VH3-L6-VH4-L7-VL4-L8-VL3-L9-CL-L10-CH1, VH3-L6-VH4-L7-VL4-L8-VL3-L9-CH1-L10-CL, VL4-L6-VL3-L7-VH3-L8-VH4-L9-CL-L10-CH1, VL4-L6-VL3-L7-VH3-L8-VH4-L9-CH1-L10-CL, VL4-L6-VH3-L7-VL3-L8-VH4-L9-CL-L10-CH1, VL4-L6-VH3-L7-VL3-L8-VH4-L9-CH1-L10-CL, VH4-L6-VL3-L7-VH3-L8-VL4-L9-CL-L10-CH1, VH4-L6-VL3-L7-VH3-L8-VL4-L9-CH1-L10-CL, VH4-L6-VH3-L7-VL3-L8-VL4-L9-CL-L10-CH1, or VH4-L6-VH3-L7-VL3- L8-VL4-L9-CH1-L10-CL.

In some aspects, an antigen binding polypeptide complex provided herein comprises a first polypeptide, and a second polypeptide; wherein the first polypeptide has a structure represented by VL1-VL2-VH2-VH1-CL-CH1-CH2-CH3, VL1-VL2-VH2-VH1-CH1-CL-CH2-CH3, VL1-VH2-VL2-VH1-CL-CH1-CH2-CH3, VL1-VH2-VL2-VH1-CH1-CL-CH2-CH3, VH1-VL2-VH2-VL1-CL-CH1-CH2-CH3, VH1-VL2-VH2-VL1-CH1-CL-CH2-CH3, VH1-VH2-VL2-VL1-CL-CH1-CH2-CH3, VH1-VH2-VL2-VL1-CH1-CL-CH2-CH3, VL2-VL1-VH1-VH2-CL-CH1-CH2-CH3, VL2-VL1-VH1-VH2-CH1-CL-CH2-CH3, VL2-VH1-VL1-VH2-CL-CH1-CH2-CH3, VL2-VH1-VL1-VH2-CH1-CL-CH2-CH3, VH2-VL1-VH1-VL2-CL-CH1-CH2-CH3, VH2-VL1-VH1-VL2-CH1-CL-CH2-CH3, VH2-VH1-VL1-VL2-CL-CH1-CH2-CH3, or VH2-VH1-VL1-VL2-CH1-CL-CH2-CH3; and wherein the second polypeptide has a structure represented by VL3-VL4-VH4-VH3-CL-CH1-CH2-CH3, VL3-VL4-VH4-VH3-CH1-CL-CH2-CH3, VL3-VH4-VL4-VH3-CL-CH1-CH2-CH3, VL3-VH4-VL4-VH3-CH1-CL-CH2-CH3, VH3-VL4-VH4-VL3-CL-CH1-CH2-CH3, VH3-VL4-VH4-VL3-CH1-CL-CH2-CH3, VH3-VH4-VL4-VL3-CL-CH1-CH2-CH3, VH3-VH4-VL4-VL3-CH1-CL-CH2-CH3, VL4-VL3-VH3-VH4-CL-CH1-CH2-CH3, VL4-VL3-VH3-VH4-CH1-CL-CH2-CH3, VL4-VH3-VL3-VH4-CL-CH1-CH2-CH3, VL4-VH3-VL3-VH4-CH1-CL-CH2-CH3, VH4-VL3-VH3-VL4-CL-CH1-CH2-CH3, VH4-VL3-VH3-VL4-CH1-CL-CH2-CH3, VH4-VH3-VL3-VL4-CL-CH1-CH2-CH3, or VH4-VH3-VL3-VL4-CH1-CL-CH2-CH3.

In some aspects, an antigen binding polypeptide complex provided herein comprises a first polypeptide, and a second polypeptide; wherein the first polypeptide has a structure represented by VL1-L1-VL2-L2-VH2-L3-VH1-L4-CL-L5-CH1-CH2-CH3, VL1-L1-VL2-L2-VH2-L3-VH1-L4-CH1- L5-CL-CH2-CH3, VL1-L1-VH2-L2-VL2-L3-VH1-L4-CL-L5-CH1-CH2-CH3, VL1-L1-VH2-L2-VL2-L3-VH1-L4-CH1-L5-CL-CH2-CH3, VH1-L1-VL2-L2-VH2-L3-VL1-L4-CL-L5-CH1-CH2-CH3, VH1-L1-VL2-L2-VH2-L3-VL1-L4-CH1-L5-CL-CH2-CH3, VH1-L1-VH2-L2-VL2-L3-VL1-L4-CL-L5-CH1-CH2-CH3, VH1-L1-VH2-L2-VL2-L3-VL1-L4-CH1-L5-CL-CH2-CH3, VL2-L1-VL1-L2-VH1-L3-VH2-L4- CL-L5-CH1-CH2-CH3, VL2-L1-VL1-L2-VH1-L3-VH2-L4-CH1-L5-CL-CH2-CH3, VL2-L1-VH1-L2-VL1-L3-VH2-L4-CL-L5-CH1-CH2-CH3, VL2-L1-VH1-L2-VL1-L3-VH2-L4-CH1-L5-CL-CH2-CH3, VH2-L1-VL1-L2-VH1-L3-VL2-L4-CL-L5-CH1-CH2-CH3, VH2-L1-VL1-L2-VH1-L3-VL2-L4-CH1-L5- CL-CH2-CH3, VH2-L1-VH1-L2-VL1-L3-VL2-L4-CL-L5-CH1-CH2-CH3, or VH2-L1-VH1-L2-VL1-L3-VL2-L4-CH1-L5-CL-CH2-CH3; and wherein the second polypeptide has a structure represented by VL3-L6-VL4-L7-VH4-L8-VH3-L9-CL-L10-CH1-CH2-CH3, VL3-L6-VL4-L7-VH4-L8-VH3-L9-CH1-L10-CL-CH2-CH3, VL3-L6-VH4-L7-VL4-L8-VH3-L9-CL-L10-CH1-CH2-CH3, VL3-L6-VH4-L7-VL4-L8-VH3-L9-CH1-L10-CL-CH2-CH3, VH3-L6-VL4-L7-VH4-L8-VL3-L9-CL-L10-CH1-CH2-CH3, VH3-L6-VL4- L7-VH4-L8-VL3-L9-CH1-L10-CL-CH2-CH3, VH3-L6-VH4-L7-VL4-L8-VL3-L9-CL-L10-CH1-CH2-CH3, VH3-L6-VH4-L7-VL4-L8-VL3-L9-CH1-L10-CL-CH2-CH3, VL4-L6-VL3-L7-VH3-L8-VH4-L9- CL-L10-CH1-CH2-CH3, VL4-L6-VL3-L7-VH3-L8-VH4-L9-CH1-L10-CL-CH2-CH3, VL4-L6-VH3-L7-VL3-L8-VH4-L9-CL-L10-CH1-CH2-CH3, VL4-L6-VH3-L7-VL3-L8-VH4-L9-CH1-L10-CL-CH2-CH3, VH4- L6-VL3-L7-VH3-L8-VL4-L9-CL-L10-CH1-CH2-CH3, VH4-L6-VL3-L7-VH3-L8-VL4-L9-CH1-L10-CL-CH2-CH3, VH4-L6-VH3-L7-VL3-L8-VL4-L9-CL-L10-CH1-CH2-CH3, or VH4-L6-VH3-L7-VL3-L8-VL4-L9-CH1-L10-CL-CH2-CH3.

In some aspects, an antigen binding polypeptide complex provided herein comprises a first polypeptide, and a second polypeptide; wherein the first polypeptide has a structure represented by VL1-VH1-VL2-CL-VH2-CH1, VL1-VH1-VL2-CH1-VH2-CL, VL1-VH1-VH2-CL-VL2-CH1, VL1-VH1-VH2-CH1-VL2-CL, VL1-VL2-VH1-CL-VH2-CH1, VL1-VL2-VH1-CH1-VH2-CL, VL1-VH2-VH1-CL-VL2-CH1, VL1-VH2-VH1-CH1-VL2-CL, VH1-VL1-VL2-CL-VH2-CH1, VH1-VL1-VL2-CH1-VH2-CL, VH1-VL1-VH2-CL-VL2-CH1, VH1-VL1-VH2-CH1-VL2-CL, VH1-VL2-VL1-CL-VH2-CH1, VH1-VL2-VL1-CH1-VH2-CL, VH1-VH2-VL1-CL-VL2-CH1, VH1-VH2-VL1-CH1-VL2-CL, VL2-VL1-VH2-CL-VH1-CH1, VL2-VL1-VH2-CH1-VH1-CL, VL2-VH1-VH2-CL-VL1-CH1, VL2-VH1-VH2-CH1-VL1-CL, VL2-VH2-VL1-CL-VH1-CH1, VL2-VH2-VL1-CH1-VH1-CL, VL2-VH2-VH1-CL-VL1-CH1, VL2-VH2-VH1-CH1-VL1-CL, VH2-VL1-VL2-CL-VH1-CH1, VH2-VL1-VL2-CH1-VH1-CL, VH2-VH1-VL2-CL-VL1-CH1, VH2-VH1-VL2-CH1-VL1-CL, VH2-VL2-VL1-CL-VH1-CH1, VH2-VL2-VL1-CH1-VH1-CL, VH2-VL2-VH1-CL-VL1-CH1, or VH2-VL2-VH1-CH1-VL1-CL; and wherein the second polypeptide has a structure represented by VL3-CL-VH3-CH1-VL4-VH4, VL3-CL-VH3-CH1-VH4-VL4, VL3-CL-VL4-CH1-VH3-VH4, VL3-CL-VH4-CH1-VH3-VL4, VL3-CH1-VH3-CL-VL4-VH4, VL3-CH1-VH3-CL-VH4-VL4, VL3-CH1-VL4-CL-VH3-VH4, VL3-CH1-VH4-CL-VH3-VL4, VH3-CL-VL3-CH1-VL4-VH4, VH3-CL-VL3-CH1-VH4-VL4, VH3-CL-VL4-CH1-VL3-VH4, VH3-CL-VH4-CH1-VL3-VL4, VH3-CH1-VL3-CL-VL4-VH4, VH3-CH1-VL3-CL-VH4-VL4, VH3-CH1-VL4-CL-VL3-VH4, VH3-CH1-VH4-CL-VL3-VL4, VL4-CL-VL3-CH1-VH4-VH3, VL4-CL-VH3-CH1-VH4-VL3, VL4-CL-VH4-CH1-VL3-VH3, VL4-CL-VH4-CH1-VH3-VL3, VL4-CH1-VL3-CL-VH4-VH3, VL4-CH1-VH3-CL-VH4-VL3, VL4-CH1-VH4-CL-VL3-VH3, VL4-CH1-VH4-CL-VH3-VL3, VH4-CL-VL3-CH1-VL4-VH3, VH4-CL-VH3-CH1-VL4-VL3, VH4-CL-VL4-CH1-VL3-VH3, VH4-CL-VL4-CH1-VH3-VL3, VH4-CH1-VL3-CL-VL4-VH3, VH4-CH1-VH3-CL-VL4-VL3, VH4-CH1-VL4-CL-VL3-VH3, or VH4-CH1-VL4-CL-VH3-VL3.

In some aspects, an antigen binding polypeptide complex provided herein comprises a first polypeptide, and a second polypeptide; wherein the first polypeptide has a structure represented by VL1-L1-VH1-L2-VL2-L3-CL-L4-VH2-L5-CH1, VL1-L1-VH1-L2-VL2-L3-CH1-L4-VH2-L5-CL, VL1-L1-VH1-L2-VH2-L3-CL-L4-VL2-L5-CH1, VL1-L1-VH1-L2-VH2-L3-CH1-L4-VL2-L5-CL, VL1-L1-VL2-L2-VH1-L3-CL-L4-VH2-L5-CH1, VL1-L1-VL2-L2-VH1-L3-CH1-L4-VH2-L5-CL, VL1-L1-VH2-L2-VH1-L3-CL-L4-VL2-L5-CH1, VL1-L1-VH2-L2-VH1-L3-CH1-L4-VL2-L5-CL, VH1-L1-VL1-L2-VL2-L3-CL-L4-VH2-L5-CH1, VH1-L1-VL1-L2-VL2-L3-CH1-L4-VH2-L5-CL, VH1-L1-VL1-L2-VH2-L3-CL-L4-VL2-L5-CH1, VH1-L1-VL1-L2-VH2-L3-CH1-L4-VL2-L5-CL, VH1-L1-VL2-L2-VL1-L3-CL-L4-VH2-L5-CH1, VH1-L1-VL2-L2-VL1-L3-CH1-L4-VH2-L5-CL, VH1-L1-VH2-L2-VL1-L3-CL-L4-VL2-L5-CH1, VH1-L1-VH2-L2-VL1-L3-CH1-L4-VL2-L5-CL, VL2-L1-VL1-L2-VH2-L3-CL-L4-VH1-L5-CH1, VL2-L1-VL1-L2-VH2-L3-CH1-L4-VH1-L5-CL, VL2-L1-VH1-L2-VH2-L3-CL-L4-VL1-L5-CH1, VL2-L1-VH1-L2-VH2-L3-CH1-L4-VL1-L5-CL, VL2-L1-VH2-L2-VL1-L3-CL-L4-VH1-L5-CH1, VL2-L1-VH2-L2-VL1-L3-CH1-L4-VH1-L5-CL, VL2-L1-VH2-L2-VH1-L3-CL-L4-VL1-L5-CH1, VL2-L1-VH2-L2-VH1-L3-CH1-L4-VL1-L5-CL, VH2-L1-VL1-L2-VL2-L3-CL-L4-VH1-L5-CH1, VH2-L1-VL1-L2-VL2-L3-CH1-L4-VH1-L5-CL, VH2-L1-VH1-L2-VL2-L3-CL-L4-VL1-L5-CH1, VH2-L1-VH1-L2-VL2-L3-CH1-L4-VL1-L5-CL, VH2-L1-VL2-L2-VL1-L3-CL-L4-VH1-L5-CH1, VH2-L1-VL2-L2-VL1-L3-CH1-L4-VH1-L5-CL, VH2-L1-VL2-L2-VH1-L3-CL-L4-VL1-L5-CH1, or VH2-L1-VL2-L2-VH1-L3-CH1-L4-VL1-L5-CL; and wherein the second polypeptide has a structure represented by VL3-L6-CL-L7-VH3-L8-CH1-L9-VL4-L10-VH4, VL3-L6-CL-L7-VH3-L8-CH1-L9-VH4-L10-VL4, VL3-L6-CL-L7-VL4-L8-CH1-L9-VH3-L10-VH4, VL3-L6-CL-L7-VH4-L8-CH1-L9-VH3-L10-VL4, VL3-L6-CH1-L7-VH3-L8-CL-L9-VL4-L10-VH4, VL3-L6-CH1-L7-VH3-L8-CL-L9-VH4-L10-VL4, VL3-L6-CH1-L7-VL4-L8-CL-L9-VH3-L10-VH4, VL3-L6-CH1-L7-VH4-L8-CL-L9-VH3-L10-VL4, VH3-L6-CL-L7- VL3-L8-CH1-L9-VL4-L10-VH4, VH3-L6-CL-L7-VL3-L8-CH1-L9-VH4-L10-VL4, VH3-L6-CL-L7-VL4-L8-CH1-L9-VL3-L10-VH4, VH3-L6-CL-L7-VH4-L8-CH1-L9-VL3-L10-VL4, VH3-L6-CH1-L7-VL3-L8-CL-L9-VL4-L10-VH4, VH3-L6-CH1-L7-VL3-L8-CL-L9-VH4-L10-VL4, VH3-L6-CH1-L7-VL4-L8-CL-L9-VL3-L10-VH4, VH3-L6-CH1-L7-VH4-L8-CL-L9-VL3-L10-VL4, VL4-L6-CL-L7-VL3-L8-CH1- L9-VH4-L10-VH3, VL4-L6-CL-L7-VH3-L8-CH1-L9-VH4-L10-VL3, VL4-L6-CL-L7-VH4-L8-CH1-L9-VL3-L10-VH3, VL4-L6-CL-L7-VH4-L8-CH1-L9-VH3-L10-VL3, VL4-L6-CH1-L7-VL3-L8-CL-L9-VH4-L10-VH3, VL4-L6-CH1-L7-VH3-L8-CL-L9-VH4-L10-VL3, VL4-L6-CH1-L7-VH4-L8-CL-L9-VL3-L10-VH3, VL4-L6-CH1-L7-VH4-L8-CL-L9-VH3-L10-VL3, VH4-L6-CL-L7-VL3-L8-CH1-L9-VL4-L10-VH3, VH4-L6-CL-L7-VH3-L8-CH1-L9-VL4-L10-VL3, VH4-L6-CL-L7-VL4-L8-CH1-L9-VL3-L10-VH3, VH4-L6-CL-L7-VL4-L8-CH1-L9-VH3-L10-VL3, VH4-L6-CH1-L7-VL3-L8-CL-L9-VL4-L10-VH3, VH4-L6-CH1-L7-VH3-L8-CL-L9-VL4-L10-VL3, VH4-L6-CH1-L7-VL4-L8-CL-L9-VL3-L10-VH3, or VH4-L6-CH1-L7-VL4-L8-CL-L9-VH3-L10-VL3.

In some aspects, an antigen binding polypeptide complex provided herein comprises a first polypeptide, and a second polypeptide; wherein the first polypeptide has a structure represented by VL1-VH1-VL2-CL-VH2-CH1-CH2-CH3, VL1-VH1-VL2-CH1-VH2-CL-CH2-CH3, VL1-VH1-VH2-CL-VL2-CH1-CH2-CH3, VL1-VH1-VH2-CH1-VL2-CL-CH2-CH3, VL1-VL2-VH1-CL-VH2-CH1-CH2-CH3, VL1-VL2-VH1-CH1-VH2-CL-CH2-CH3, VL1-VH2-VH1-CL-VL2-CH1-CH2-CH3, VL1-VH2-VH1-CH1-VL2-CL-CH2-CH3, VH1-VL1-VL2-CL-VH2-CH1-CH2-CH3, VH1-VL1-VL2-CH1-VH2-CL-CH2-CH3, VH1-VL1-VH2-CL-VL2-CH1-CH2-CH3, VH1-VL1-VH2-CH1-VL2-CL-CH2-CH3, VH1-VL2-VL1-CL-VH2-CH1-CH2-CH3, VH1-VL2-VL1-CH1-VH2-CL-CH2-CH3, VH1-VH2-VL1-CL-VL2-CH1-CH2-CH3, VH1-VH2-VL1-CH1-VL2-CL-CH2-CH3, VL2-VL1-VH2-CL-VH1-CH1-CH2-CH3, VL2-VL1-VH2-CH1-VH1-CL-CH2-CH3, VL2-VH1-VH2-CL-VL1-CH1-CH2-CH3, VL2-VH1-VH2-CH1-VL1-CL-CH2-CH3, VL2-VH2-VL1-CL-VH1-CH1-CH2-CH3, VL2-VH2-VL1-CH1-VH1-CL-CH2-CH3, VL2-VH2-VH1-CL-VL1-CH1-CH2-CH3, VL2-VH2-VH1-CH1-VL1-CL-CH2-CH3, VH2-VL1-VL2-CL-VH1-CH1-CH2-CH3, VH2-VL1-VL2-CH1-VH1-CL-CH2-CH3, VH2-VH1-VL2-CL-VL1-CH1-CH2-CH3, VH2-VH1-VL2-CH1-VL1-CL-CH2-CH3, VH2-VL2-VL1-CL-VH1-CH1-CH2-CH3, VH2-VL2-VL1-CH1-VH1-CL-CH2-CH3, VH2-VL2-VH1-CL-VL1-CH1-CH2-CH3, or VH2-VL2-VH1-CH1-VL1-CL-CH2-CH3; and wherein the second polypeptide has a structure represented by VL3-CL-VH3-CH1-VL4-VH4-CH2-CH3, VL3-CL-VH3-CH1-VH4-VL4-CH2-CH3, VL3-CL-VL4-CH1-VH3-VH4-CH2-CH3, VL3-CL-VH4-CH1-VH3-VL4-CH2-CH3, VL3-CH1-VH3-CL-VL4-VH4-CH2-CH3, VL3-CH1-VH3-CL-VH4-VL4-CH2-CH3, VL3-CH1-VL4-CL-VH3-VH4-CH2-CH3, VL3-CH1-VH4-CL-VH3-VL4-CH2-CH3, VH3-CL-VL3-CH1-VL4-VH4-CH2-CH3, VH3-CL-VL3-CH1-VH4-VL4-CH2-CH3, VH3-CL-VL4-CH1-VL3-VH4-CH2-CH3, VH3-CL-VH4-CH1-VL3-VL4-CH2-CH3, VH3-CH1-VL3-CL-VL4-VH4-CH2-CH3, VH3-CH1-VL3-CL-VH4-VL4-CH2-CH3, VH3-CH1-VL4-CL-VL3-VH4-CH2-CH3, VH3-CH1-VH4-CL-VL3-VL4-CH2-CH3, VL4-CL-VL3-CH1-VH4-VH3-CH2-CH3, VL4-CL-VH3-CH1-VH4-VL3-CH2-CH3, VL4-CL-VH4-CH1-VL3-VH3-CH2-CH3, VL4-CL-VH4-CH1-VH3-VL3-CH2-CH3, VL4-CH1-VL3-CL-VH4-VH3-CH2-CH3, VL4-CH1-VH3-CL-VH4-VL3-CH2-CH3, VL4-CH1-VH4-CL-VL3-VH3-CH2-CH3, VL4-CH1-VH4-CL-VH3-VL3-CH2-CH3, VH4-CL-VL3-CH1-VL4-VH3-CH2-CH3, VH4-CL-VH3-CH1-VL4-VL3-CH2-CH3, VH4-CL-VL4-CH1-VL3-VH3-CH2-CH3, VH4-CL-VL4-CH1-VH3-VL3-CH2-CH3, VH4-CH1-VL3-CL-VL4-VH3-CH2-CH3, VH4-CH1-VH3-CL-VL4-VL3-CH2-CH3, VH4-CH1-VL4-CL-VL3-VH3-CH2-CH3, or VH4-CH1-VL4-CL-VH3-VL3-CH2-CH3.

In some aspects, an antigen binding polypeptide complex provided herein comprises a first polypeptide, and a second polypeptide; wherein the first polypeptide has a structure represented by VL1-L1-VH1-L2-VL2-L3-CL-L4-VH2-L5-CH1-CH2-CH3, VL1-L1-VH1-L2-VL2-L3-CH1-L4-VH2- L5-CL-CH2-CH3, VL1-L1-VH1-L2-VH2-L3-CL-L4-VL2-L5-CH1-CH2-CH3, VL1-L1-VH1-L2-VH2-L3-CH1-L4-VL2-L5-CL-CH2-CH3, VL1-L1-VL2-L2-VH1-L3-CL-L4-VH2-L5-CH1-CH2-CH3, VL1-L1-VL2-L2-VH1-L3-CH1-L4-VH2-L5-CL-CH2-CH3, VL1-L1-VH2-L2-VH1-L3-CL-L4-VL2-L5-CH1-CH2-CH3, VL1-L1-VH2-L2-VH1-L3-CH1-L4-VL2-L5-CL-CH2-CH3, VH1-L1-VL1-L2-VL2-L3-CL-L4-VH2-L5-CH1-CH2-CH3, VH1-L1-VL1-L2-VL2-L3-CH1-L4-VH2-L5-CL-CH2-CH3, VH1-L1-VL1-L2-VH2-L3-CL-L4-VL2-L5-CH1-CH2-CH3, VH1-L1-VL1-L2-VH2-L3-CH1-L4-VL2-L5-CL-CH2-CH3, VH1-L1-VL2-L2-VL1-L3-CL-L4-VH2-L5-CH1-CH2-CH3, VH1-L1-VL2-L2-VL1-L3-CH1-L4-VH2-L5- CL-CH2-CH3, VH1-L1-VH2-L2-VL1-L3-CL-L4-VL2-L5-CH1-CH2-CH3, VH1-L1-VH2-L2-VL1-L3-CH1-L4-VL2-L5-CL-CH2-CH3, VL2-L1-VL1-L2-VH2-L3-CL-L4-VH1-L5-CH1-CH2-CH3, VL2-L1-VL1-L2-VH2-L3-CH1-L4-VH1-L5-CL-CH2-CH3, VL2-L1-VH1-L2-VH2-L3-CL-L4-VL1-L5-CH1-CH2-CH3, VL2-L1-VH1-L2-VH2-L3-CH1-L4-VL1-L5-CL-CH2-CH3, VL2-L1-VH2-L2-VL1-L3-CL-L4-VH1-L5-CH1-CH2-CH3, VL2-L1-VH2-L2-VL1-L3-CH1-L4-VH1-L5-CL-CH2-CH3, VL2-L1-VH2-L2-VH1-L3-CL-L4-VL1-L5-CH1-CH2-CH3, VL2-L1-VH2-L2-VH1-L3-CH1-L4-VL1-L5-CL-CH2-CH3, VH2-L1-VL1-L2-VL2-L3-CL-L4-VH1-L5-CH1-CH2-CH3, VH2-L1-VL1-L2-VL2-L3-CH1-L4-VH1-L5- CL-CH2-CH3, VH2-L1-VH1-L2-VL2-L3-CL-L4-VL1-L5-CH1-CH2-CH3, VH2-L1-VH1-L2-VL2-L3-CH1-L4-VL1-L5-CL-CH2-CH3, VH2-L1-VL2-L2-VL1-L3-CL-L4-VH1-L5-CH1-CH2-CH3, VH2-L1-VL2-L2-VL1-L3-CH1-L4-VH1-L5-CL-CH2-CH3, VH2-L1-VL2-L2-VH1-L3-CL-L4-VL1-L5-CH1-CH2-CH3, or VH2-L1-VL2-L2-VH1-L3-CH1-L4-VL1-L5-CL-CH2-CH3; and wherein the second polypeptide has a structure represented by VL3-L6-CL-L7-VH3-L8-CH1-L9-VL4-L10-VH4-CH2-CH3, VL3-L6-CL-L7-VH3-L8-CH1-L9-VH4-L10-VL4-CH2-CH3, VL3-L6-CL-L7-VL4-L8-CH1-L9-VH3-L10-VH4-CH2-CH3, VL3-L6-CL-L7-VH4-L8-CH1-L9-VH3-L10-VL4-CH2-CH3, VL3-L6-CH1-L7-VH3-L8-CL-L9-VL4-L10-VH4-CH2-CH3, VL3-L6-CH1-L7-VH3-L8-CL-L9-VH4-L10-VL4-CH2-CH3, VL3-L6-CH1-L7-VL4-L8-CL-L9-VH3-L10-VH4-CH2-CH3, VL3-L6-CH1-L7-VH4-L8-CL-L9-VH3-L10-VL4-CH2-CH3, VH3- L6-CL-L7-VL3-L8-CH1-L9-VL4-L10-VH4-CH2-CH3, VH3-L6-CL-L7-VL3-L8-CH1-L9-VH4-L10-VL4-CH2-CH3, VH3-L6-CL-L7-VL4-L8-CH1-L9-VL3-L10-VH4-CH2-CH3, VH3-L6-CL-L7-VH4-L8-CH1-L9-VL3-L10-VL4-CH2-CH3, VH3-L6-CH1-L7-VL3-L8-CL-L9-VL4-L10-VH4-CH2-CH3, VH3-L6-CH1-L7-VL3-L8-CL-L9-VH4-L10-VL4-CH2-CH3, VH3-L6-CH1-L7-VL4-L8-CL-L9-VL3-L10-VH4-CH2-CH3, VH3-L6-CH1-L7-VH4-L8-CL-L9-VL3-L10-VL4-CH2-CH3, VL4-L6-CL-L7-VL3-L8-CH1-L9-VH4-L10-VH3-CH2-CH3, VL4-L6-CL-L7-VH3-L8-CH1-L9-VH4-L10-VL3-CH2-CH3, VL4-L6-CL- L7-VH4-L8-CH1-L9-VL3-L10-VH3-CH2-CH3, VL4-L6-CL-L7-VH4-L8-CH1-L9-VH3-L10-VL3-CH2-CH3, VL4-L6-CH1-L7-VL3-L8-CL-L9-VH4-L10-VH3-CH2-CH3, VL4-L6-CH1-L7-VH3-L8-CL-L9-VH4-L10-VL3-CH2-CH3, VL4-L6-CH1-L7-VH4-L8-CL-L9-VL3-L10-VH3-CH2-CH3, VL4-L6-CH1-L7-VH4-L8-CL-L9-VH3-L10-VL3-CH2-CH3, VH4-L6-CL-L7-VL3-L8-CH1-L9-VL4-L10-VH3-CH2-CH3, VH4- L6-CL-L7-VH3-L8-CH1-L9-VL4-L10-VL3-CH2-CH3, VH4-L6-CL-L7-VL4-L8-CH1-L9-VL3-L10-VH3-CH2-CH3, VH4-L6-CL-L7-VL4-L8-CH1-L9-VH3-L10-VL3-CH2-CH3, VH4-L6-CH1-L7-VL3-L8-CL-L9-VL4-L10-VH3-CH2-CH3, VH4-L6-CH1-L7-VH3-L8-CL-L9-VL4-L10-VL3-CH2-CH3, VH4-L6-CH1-L7-VL4-L8-CL-L9-VL3-L10-VH3-CH2-CH3, or VH4-L6-CH1-L7-VL4-L8-CL-L9-VH3-L10- VL3-CH2-CH3.

In some aspects, an antigen binding polypeptide complex provided herein comprises a first polypeptide, and a second polypeptide; wherein the first polypeptide has a structure represented by VL1-CL-VH1-CH1-VL2-VH2, VL1-CL-VH1-CH1-VH2-VL2, VL1-CL-VL2-CH1-VH1-VH2, VL1-CL-VH2-CH1-VH1-VL2, VL1-CH1-VH1-CL-VL2-VH2, VL1-CH1-VH1-CL-VH2-VL2, VL1-CH1-VL2-CL-VH1-VH2, VL1-CH1-VH2-CL-VH1-VL2, VH1-CL-VL1-CH1-VL2-VH2, VH1-CL-VL1-CH1-VH2-VL2, VH1-CL-VL2-CH1-VL1-VH2, VH1-CL-VH2-CH1-VL1-VL2, VH1-CH1-VL1-CL-VL2-VH2, VH1-CH1-VL1-CL-VH2-VL2, VH1-CH1-VL2-CL-VL1-VH2, VH1-CH1-VH2-CL-VL1-VL2, VL2-CL-VL1-CH1-VH2-VH1, VL2-CL-VH1-CH1-VH2-VL1, VL2-CL-VH2-CH1-VL1-VH1, VL2-CL-VH2-CH1-VH1-VL1, VL2-CH1-VL1-CL-VH2-VH1, VL2-CH1-VH1-CL-VH2-VL1, VL2-CH1-VH2-CL-VL1-VH1, VL2-CH1-VH2-CL-VH1-VL1, VH2-CL-VL1-CH1-VL2-VH1, VH2-CL-VH1-CH1-VL2-VL1, VH2-CL-VL2-CH1-VL1-VH1, VH2-CL-VL2-CH1-VH1-VL1, VH2-CH1-VL1-CL-VL2-VH1, VH2-CH1-VH1-CL-VL2-VL1, VH2-CH1-VL2-CL-VL1-VH1, or VH2-CH1-VL2-CL-VH1-VL1; and wherein the second polypeptide has a structure represented by VL3-VH3-VL4-CL-VH4-CH1, VL3-VH3-VL4-CH1-VH4-CL, VL3-VH3-VH4-CL-VL4-CH1, VL3-VH3-VH4-CH1-VL4-CL, VL3-VL4-VH3-CL-VH4-CH1, VL3-VL4-VH3-CH1-VH4-CL, VL3-VH4-VH3-CL-VL4-CH1, VL3-VH4-VH3-CH1-VL4-CL, VH3-VL3-VL4-CL-VH4-CH1, VH3-VL3-VL4-CH1-VH4-CL, VH3-VL3-VH4-CL-VL4-CH1, VH3-VL3-VH4-CH1-VL4-CL, VH3-VL4-VL3-CL-VH4-CH1, VH3-VL4-VL3-CH1-VH4-CL, VH3-VH4-VL3-CL-VL4-CH1, VH3-VH4-VL3-CH1-VL4-CL, VL4-VL3-VH4-CL-VH3-CH1, VL4-VL3-VH4-CH1-VH3-CL, VL4-VH3-VH4-CL-VL3-CH1, VL4-VH3-VH4-CH1-VL3-CL, VL4-VH4-VL3-CL-VH3-CH1, VL4-VH4-VL3-CH1-VH3-CL, VL4-VH4-VH3-CL-VL3-CH1, VL4-VH4-VH3-CH1-VL3-CL, VH4-VL3-VL4-CL-VH3-CH1, VH4-VL3-VL4-CH1-VH3-CL, VH4-VH3-VL4-CL-VL3-CH1, VH4-VH3-VL4-CH1-VL3-CL, VH4-VL4-VL3-CL-VH3-CH1, VH4-VL4-VL3-CH1-VH3-CL, VH4-VL4-VH3-CL-VL3-CH1, or VH4-VL4-VH3-CH1-VL3-CL.

In some aspects, an antigen binding polypeptide complex provided herein comprises a first polypeptide, and a second polypeptide; wherein the first polypeptide has a structure represented by VL1-L1-CL-L2-VH1-L3-CH1-L4-VL2-L5-VH2, VL1-L1-CL-L2-VH1-L3-CH1-L4-VH2-L5-VL2, VL1-L1-CL-L2-VL2-L3-CH1-L4-VH1-L5-VH2, VL1-L1-CL-L2-VH2-L3-CH1-L4-VH1-L5-VL2, VL1-L1-CH1-L2-VH1-L3-CL-L4-VL2-L5-VH2, VL1-L1-CH1-L2-VH1-L3-CL-L4-VH2-L5-VL2, VL1-L1-CH1-L2-VL2-L3-CL-L4-VH1-L5-VH2, VL1-L1-CH1-L2-VH2-L3-CL-L4-VH1-L5-VL2, VH1-L1-CL-L2-VL1-L3-CH1-L4-VL2-L5-VH2, VH1-L1-CL-L2-VL1-L3-CH1-L4-VH2-L5-VL2, VH1-L1-CL-L2-VL2-L3-CH1-L4-VL1-L5-VH2, VH1-L1-CL-L2-VH2-L3-CH1-L4-VL1-L5-VL2, VH1-L1-CH1-L2-VL1-L3- CL-L4-VL2-L5-VH2, VH1-L1-CH1-L2-VL1-L3-CL-L4-VH2-L5-VL2, VH1-L1-CH1-L2-VL2-L3-CL-L4-VL1-L5-VH2, VH1-L1-CH1-L2-VH2-L3-CL-L4-VL1-L5-VL2, VL2-L1-CL-L2-VL1-L3-CH1-L4-VH2-L5-VH1, VL2-L1-CL-L2-VH1-L3-CH1-L4-VH2-L5-VL1, VL2-L1-CL-L2-VH2-L3-CH1-L4-VL1-L5-VH1, VL2-L1-CL-L2-VH2-L3-CH1-L4-VH1-L5-VL1, VL2-L1-CH1-L2-VL1-L3-CL-L4-VH2-L5-VH1, VL2-L1-CH1-L2-VH1-L3-CL-L4-VH2-L5-VL1, VL2-L1-CH1-L2-VH2-L3-CL-L4-VL1-L5-VH1, VL2-L1-CH1-L2-VH2-L3-CL-L4-VH1-L5-VL1, VH2-L1-CL-L2-VL1-L3-CH1-L4-VL2-L5-VH1, VH2-L1-CL-L2-VH1-L3-CH1-L4-VL2-L5-VL1, VH2-L1-CL-L2-VL2-L3-CH1-L4-VL1-L5-VH1, VH2-L1-CL- L2-VL2-L3-CH1-L4-VH1-L5-VL1, VH2-L1-CH1-L2-VL1-L3-CL-L4-VL2-L5-VH1, VH2-L1-CH1-L2-VH1-L3-CL-L4-VL2-L5-VL1, VH2-L1-CH1-L2-VL2-L3-CL-L4-VL1-L5-VH1, or VH2-L1-CH1-L2-VL2-L3-CL-L4-VH1-L5-VL1; and wherein the second polypeptide has a structure represented by VL3-L6-VH3-L7-VL4-L8-CL-L9-VH4-L10-CH1, VL3-L6-VH3-L7-VL4-L8-CH1-L9-VH4-L10-CL, VL3-L6-VH3-L7-VH4-L8-CL-L9-VL4-L10-CH1, VL3-L6-VH3-L7-VH4-L8-CH1-L9-VL4-L10-CL, VL3-L6-VL4-L7-VH3-L8-CL-L9-VH4-L10-CH1, VL3-L6-VL4-L7-VH3-L8-CH1-L9-VH4-L10-CL, VL3-L6-VH4-L7-VH3-L8-CL-L9-VL4-L10-CH1, VL3-L6-VH4-L7-VH3-L8-CH1-L9-VL4-L10-CL, VH3-L6-VL3-L7-VL4-L8-CL-L9-VH4-L10-CH1, VH3-L6-VL3-L7-VL4-L8-CH1-L9-VH4-L10-CL, VH3-L6-VL3-L7-VH4-L8-CL-L9-VL4-L10-CH1, VH3-L6-VL3-L7-VH4-L8-CH1-L9-VL4-L10-CL, VH3-L6-VL4-L7-VL3-L8-CL-L9-VH4-L10-CH1, VH3-L6-VL4-L7-VL3-L8-CH1-L9-VH4-L10-CL, VH3-L6-VH4-L7-VL3-L8-CL-L9-VL4-L10-CH1, VH3-L6-VH4-L7-VL3-L8-CH1-L9-VL4-L10-CL, VL4-L6-VL3-L7-VH4-L8-CL-L9-VH3-L10-CH1, VL4-L6-VL3-L7-VH4-L8-CH1-L9-VH3-L10-CL, VL4-L6-VH3-L7-VH4-L8-CL-L9-VL3-L10-CH1, VL4-L6-VH3-L7-VH4-L8-CH1-L9-VL3-L10-CL, VL4-L6-VH4-L7-VL3-L8-CL-L9-VH3-L10-CH1, VL4-L6-VH4-L7-VL3-L8-CH1-L9-VH3-L10-CL, VL4-L6-VH4-L7-VH3-L8-CL- L9-VL3-L10-CH1, VL4-L6-VH4-L7-VH3-L8-CH1-L9-VL3-L10-CL, VH4-L6-VL3-L7-VL4-L8-CL-L9-VH3-L10-CH1, VH4-L6-VL3-L7-VL4-L8-CH1-L9-VH3-L10-CL, VH4-L6-VH3-L7-VL4-L8-CL-L9-VL3-L10-CH1, VH4-L6-VH3-L7-VL4-L8-CH1-L9-VL3-L10-CL, VH4-L6-VL4-L7-VL3-L8-CL-L9-VH3-L10-CH1, VH4-L6-VL4-L7-VL3-L8-CH1-L9-VH3-L10-CL, VH4-L6-VL4-L7-VH3-L8-CL-L9-VL3-L10-CH1, or VH4-L6-VL4-L7-VH3-L8-CH1-L9-VL3-L10-CL.

In some aspects, an antigen binding polypeptide complex provided herein comprises a first polypeptide, and a second polypeptide; wherein the first polypeptide has a structure represented by VL1-CL-VH1-CH1-VL2-VH2-CH2-CH3, VL1-CL-VH1-CH1-VH2-VL2-CH2-CH3, VL1-CL-VL2-CH1-VH1-VH2-CH2-CH3, VL1-CL-VH2-CH1-VH1-VL2-CH2-CH3, VL1-CH1-VH1-CL-VL2-VH2-CH2-CH3, VL1-CH1-VH1-CL-VH2-VL2-CH2-CH3, VL1-CH1-VL2-CL-VH1-VH2-CH2-CH3, VL1-CH1-VH2-CL-VH1-VL2-CH2-CH3, VH1-CL-VL1-CH1-VL2-VH2-CH2-CH3, VH1-CL-VL1-CH1-VH2-VL2-CH2-CH3, VH1-CL-VL2-CH1-VL1-VH2-CH2-CH3, VH1-CL-VH2-CH1-VL1-VL2-CH2-CH3, VH1-CH1-VL1-CL-VL2-VH2-CH2-CH3, VH1-CH1-VL1-CL-VH2-VL2-CH2-CH3, VH1-CH1-VL2-CL-VL1-VH2-CH2-CH3, VH1-CH1-VH2-CL-VL1-VL2-CH2-CH3, VL2-CL-VL1-CH1-VH2-VH1-CH2-CH3, VL2-CL-VH1-CH1-VH2-VL1-CH2-CH3, VL2-CL-VH2-CH1-VL1-VH1-CH2-CH3, VL2-CL-VH2-CH1-VH1-VL1-CH2-CH3, VL2-CH1-VL1-CL-VH2-VH1-CH2-CH3, VL2-CH1-VH1-CL-VH2-VL1-CH2-CH3, VL2-CH1-VH2-CL-VL1-VH1-CH2-CH3, VL2-CH1-VH2-CL-VH1-VL1-CH2-CH3, VH2-CL-VL1-CH1-VL2-VH1-CH2-CH3, VH2-CL-VH1-CH1-VL2-VL1-CH2-CH3, VH2-CL-VL2-CH1-VL1-VH1-CH2-CH3, VH2-CL-VL2-CH1-VH1-VL1-CH2-CH3, VH2-CH1-VL1-CL-VL2-VH1-CH2-CH3, VH2-CH1-VH1-CL-VL2-VL1-CH2-CH3, VH2-CH1-VL2-CL-VL1-VH1-CH2-CH3, or VH2-CH1-VL2-CL-VH1-VL1-CH2-CH3; and wherein the second polypeptide has a structure represented by VL3-VH3-VL4-CL-VH4-CH1-CH2-CH3, VL3-VH3-VL4-CH1-VH4-CL-CH2-CH3, VL3-VH3-VH4-CL-VL4-CH1-CH2-CH3, VL3-VH3-VH4-CH1-VL4-CL-CH2-CH3, VL3-VL4-VH3-CL-VH4-CH1-CH2-CH3, VL3-VL4-VH3-CH1-VH4-CL-CH2-CH3, VL3-VH4-VH3-CL-VL4-CH1-CH2-CH3, VL3-VH4-VH3-CH1-VL4-CL-CH2-CH3, VH3-VL3-VL4-CL-VH4-CH1-CH2-CH3, VH3-VL3-VL4-CH1-VH4-CL-CH2-CH3, VH3-VL3-VH4-CL-VL4-CH1-CH2-CH3, VH3-VL3-VH4-CH1-VL4-CL-CH2-CH3, VH3-VL4-VL3-CL-VH4-CH1-CH2-CH3, VH3-VL4-VL3-CH1-VH4-CL-CH2-CH3, VH3-VH4-VL3-CL-VL4-CH1-CH2-CH3, VH3-VH4-VL3-CH1-VL4-CL-CH2-CH3, VL4-VL3-VH4-CL-VH3-CH1-CH2-CH3, VL4-VL3-VH4-CH1-VH3-CL-CH2-CH3, VL4-VH3-VH4-CL-VL3-CH1-CH2-CH3, VL4-VH3-VH4-CH1-VL3-CL-CH2-CH3, VL4-VH4-VL3-CL-VH3-CH1-CH2-CH3, VL4-VH4-VL3-CH1-VH3-CL-CH2-CH3, VL4-VH4-VH3-CL-VL3-CH1-CH2-CH3, VL4-VH4-VH3-CH1-VL3-CL-CH2-CH3, VH4-VL3-VL4-CL-VH3-CH1-CH2-CH3, VH4-VL3-VL4-CH1-VH3-CL-CH2-CH3, VH4-VH3-VL4-CL-VL3-CH1-CH2-CH3, VH4-VH3-VL4-CH1-VL3-CL-CH2-CH3, VH4-VL4-VL3-CL-VH3-CH1-CH2-CH3, VH4-VL4-VL3-CH1-VH3-CL-CH2-CH3, VH4-VL4-VH3-CL-VL3-CH1-CH2-CH3, or VH4-VL4-VH3-CH1-VL3-CL-CH2-CH3.

In some aspects, an antigen binding polypeptide complex provided herein comprises a first polypeptide, and a second polypeptide; wherein the first polypeptide has a structure represented by VL1-L1-CL-L2-VH1-L3-CH1-L4-VL2-L5-VH2-CH2-CH3, VL1-L1-CL-L2-VH1-L3-CH1-L4-VH2-L5-VL2-CH2-CH3, VL1-L1-CL-L2-VL2-L3-CH1-L4-VH1-L5-VH2-CH2-CH3, VL1-L1-CL-L2-VH2-L3-CH1-L4-VH1-L5-VL2-CH2-CH3, VL1-L1-CH1-L2-VH1-L3-CL-L4-VL2-L5-VH2-CH2-CH3, VL1-L1-CH1-L2-VH1-L3-CL-L4-VH2-L5-VL2-CH2-CH3, VL1-L1-CH1-L2-VL2-L3-CL-L4-VH1-L5-VH2-CH2-CH3, VL1-L1-CH1-L2-VH2-L3-CL-L4-VH1-L5-VL2-CH2-CH3, VH1-L1-CL-L2-VL1-L3-CH1-L4-VL2-L5-VH2-CH2-CH3, VH1-L1-CL-L2-VL1-L3-CH1-L4-VH2-L5-VL2-CH2-CH3, VH1-L1-CL-L2-VL2-L3-CH1-L4-VL1-L5-VH2-CH2-CH3, VH1-L1-CL-L2-VH2-L3-CH1-L4-VL1-L5-VL2-CH2-CH3, VH1-L1-CH1-L2-VL1-L3-CL-L4-VL2-L5-VH2-CH2-CH3, VH1-L1-CH1-L2-VL1-L3-CL-L4-VH2-L5-VL2-CH2-CH3, VH1-L1-CH1-L2-VL2-L3-CL-L4-VL1-L5-VH2-CH2-CH3, VH1-L1-CH1-L2-VH2-L3-CL-L4-VL1-L5-VL2-CH2-CH3, VL2-L1-CL-L2-VL1-L3-CH1-L4-VH2-L5-VH1-CH2-CH3, VL2-L1-CL-L2-VH1-L3-CH1-L4-VH2-L5-VL1-CH2-CH3, VL2-L1-CL-L2-VH2-L3-CH1-L4-VL1-L5-VH1-CH2-CH3, VL2-L1-CL-L2-VH2-L3-CH1-L4-VH1-L5-VL1-CH2-CH3, VL2-L1-CH1-L2-VL1-L3-CL-L4-VH2-L5-VH1-CH2-CH3, VL2-L1-CH1-L2-VH1-L3-CL-L4-VH2-L5-VL1-CH2-CH3, VL2-L1-CH1-L2-VH2-L3-CL-L4-VL1-L5-VH1-CH2-CH3, VL2-L1-CH1-L2-VH2-L3-CL-L4-VH1-L5-VL1-CH2-CH3, VH2-L1-CL-L2-VL1-L3-CH1-L4-VL2-L5-VH1-CH2-CH3, VH2-L1-CL-L2-VH1-L3-CH1-L4-VL2-L5-VL1-CH2-CH3, VH2-L1-CL-L2-VL2-L3-CH1-L4-VL1-L5-VH1-CH2-CH3, VH2-L1-CL-L2-VL2-L3-CH1-L4-VH1-L5-VL1-CH2-CH3, VH2-L1-CH1-L2-VL1-L3-CL-L4-VL2-L5-VH1-CH2-CH3, VH2-L1-CH1-L2-VH1-L3-CL-L4-VL2-L5-VL1-CH2-CH3, VH2-L1-CH1-L2-VL2-L3-CL-L4-VL1-L5-VH1-CH2-CH3, or VH2-L1-CH1-L2-VL2-L3-CL-L4-VH1-L5-VL1-CH2-CH3; and wherein the second polypeptide has a structure represented by VL3-L6-VH3-L7-VL4-L8-CL-L9-VH4-L10-CH1-CH2-CH3, VL3-L6-VH3-L7-VL4-L8-CH1-L9-VH4-L10-CL-CH2-CH3, VL3-L6-VH3-L7-VH4-L8-CL-L9-VL4-L10-CH1-CH2-CH3, VL3-L6-VH3-L7- VH4-L8-CH1-L9-VL4-L10-CL-CH2-CH3, VL3-L6-VL4-L7-VH3-L8-CL-L9-VH4-L10-CH1-CH2-CH3, VL3-L6-VL4-L7-VH3-L8-CH1-L9-VH4-L10-CL-CH2-CH3, VL3-L6-VH4-L7-VH3-L8-CL-L9-VL4-L10-CH1-CH2-CH3, VL3-L6-VH4-L7-VH3-L8-CH1-L9-VL4-L10-CL-CH2-CH3, VH3-L6-VL3-L7-VL4-L8-CL-L9-VH4-L10-CH1-CH2-CH3, VH3-L6-VL3-L7-VL4-L8-CH1-L9-VH4-L10-CL-CH2-CH3, VH3-L6-VL3-L7-VH4-L8-CL-L9-VL4-L10-CH1-CH2-CH3, VH3-L6-VL3-L7-VH4-L8-CH1-L9-VL4-L10-CL-CH2-CH3, VH3-L6-VL4-L7-VL3-L8-CL-L9-VH4-L10-CH1-CH2-CH3, VH3-L6-VL4-L7-VL3-L8- CH1-L9-VH4-L10-CL-CH2-CH3, VH3-L6-VH4-L7-VL3-L8-CL-L9-VL4-L10-CH1-CH2-CH3, VH3-L6- VH4-L7-VL3-L8-CH1-L9-VL4-L10-CL-CH2-CH3, VL4-L6-VL3-L7-VH4-L8-CL-L9-VH3-L10-CH1-CH2-CH3, VL4-L6-VL3-L7-VH4-L8-CH1-L9-VH3-L10-CL-CH2-CH3, VL4-L6-VH3-L7-VH4-L8-CL-L9-VL3-L10-CH1-CH2-CH3, VL4-L6-VH3-L7-VH4-L8-CH1-L9-VL3-L10-CL-CH2-CH3, VL4-L6-VH4-L7-VL3-L8-CL-L9-VH3-L10-CH1-CH2-CH3, VL4-L6-VH4-L7-VL3-L8-CH1-L9-VH3-L10-CL-CH2-CH3, VL4-L6-VH4-L7-VH3-L8-CL-L9-VL3-L10-CH1-CH2-CH3, VL4-L6-VH4-L7-VH3-L8-CH1-L9- VL3-L10-CL-CH2-CH3, VH4-L6-VL3-L7-VL4-L8-CL-L9-VH3-L10-CH1-CH2-CH3, VH4-L6-VL3-L7- VL4-L8-CH1-L9-VH3-L10-CL-CH2-CH3, VH4-L6-VH3-L7-VL4-L8-CL-L9-VL3-L10-CH1-CH2-CH3, VH4-L6-VH3-L7-VL4-L8-CH1-L9-VL3-L10-CL-CH2-CH3, VH4-L6-VL4-L7-VL3-L8-CL-L9-VH3-L10-CH1-CH2-CH3, VH4-L6-VL4-L7-VL3-L8-CH1-L9-VH3-L10-CL-CH2-CH3, VH4-L6-VL4-L7-VH3-L8-CL-L9-VL3-L10-CH1-CH2-CH3, or VH4-L6-VL4-L7-VH3-L8-CH1-L9-VL3-L10-CL-CH2-CH3.

In some aspects, VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VL4 is a fourth immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen; CH1 is an immunoglobulin heavy chain constant region 1; CL is an immunoglobulin light chain constant region; and L1-L10 are optional amino acid linkers. In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the VL1 and the VL2 each comprise a CDR1, a CDR2, and a CDR3 that are the same. In some aspects, the VL1 and the VL2 each comprise a CDR1, a CDR2, and a CDR3 that are different. In some aspects, the VL1 and the VL3 each comprise a CDR1, a CDR2, and a CDR3 that are the same. In some aspects, the VL1 and the VL3 each comprise a CDR1, a CDR2, and a CDR3 that are different. In some aspects, the VL1 and the VL4 each comprise a CDR1, a CDR2, and a CDR3 that are the same. In some aspects, the VL1 and the VL4 each comprise a CDR1, a CDR2, and a CDR3 that are different. In some aspects, the VL2 and the VL3 each comprise a CDR1, a CDR2, and a CDR3 that are the same. In some aspects, the VL2 and the VL3 each comprise a CDR1, a CDR2, and a CDR3 that are different. In some aspects, the VL2 and the VL4 each comprise a CDR1, a CDR2, and a CDR3 that are the same. In some aspects, the VL2 and the VL4 each comprise a CDR1, a CDR2, and a CDR3 that are different. In some aspects, the VL3 and the VL4 each comprise a CDR1, a CDR2, and a CDR3 that are the same. In some aspects, the VL3 and the VL4 each comprise a CDR1, a CDR2, and a CDR3 that are different.

In some aspects, the VL1 and the VL2 each comprise an amino acid sequence, wherein the amino acid sequence comprised in VL1 and the amino acid sequence comprised in VL2 are the same. In some aspects, the VL1 and the VL2 each comprise an amino acid sequence, wherein the amino acid sequence comprised in VL1 and the amino acid sequence comprised in VL2 are different. In some aspects, the VL1 and the VL3 each comprise an amino acid sequence, wherein the amino acid sequence comprised in VL1 and the amino acid sequence comprised in VL3 are the same. In some aspects, the VL1 and the VL3 each comprise an amino acid sequence, wherein the amino acid sequence comprised in VL1 and the amino acid sequence comprised in VL3 are different. In some aspects, the VL1 and the VL4 each comprise an amino acid sequence, wherein the amino acid sequence comprised in VL1 and the amino acid sequence comprised in VL4 are the same. In some aspects, the VL1 and the VL4 each comprise an amino acid sequence, wherein the amino acid sequence comprised in VL1 and the amino acid sequence comprised in VL4 are different. In some aspects, the VL2 and the VL3 each comprise an amino acid sequence, wherein the amino acid sequence comprised in VL2 and the amino acid sequence comprised in VL3 are the same. In some aspects, the VL2 and the VL3 each comprise an amino acid sequence, wherein the amino acid sequence comprised in VL2 and the amino acid sequence comprised in VL3 are different. In some aspects, the VL2 and the VL4 each comprise an amino acid sequence, wherein the amino acid sequence comprised in VL2 and the amino acid sequence comprised in VL4 are the same. In some aspects, the VL2 and the VL4 each comprise an amino acid sequence, wherein the amino acid sequence comprised in VL2 and the amino acid sequence comprised in VL4 are different. In some aspects, the VL3 and the VL4 each comprise an amino acid sequence, wherein the amino acid sequence comprised in VL3 and the amino acid sequence comprised in VL4 are the same. In some aspects, the VL3 and the VL4 each comprise an amino acid sequence, wherein the amino acid sequence comprised in VL3 and the amino acid sequence comprised in VL4 are different.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CDIla, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxylesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptide complexes provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, eecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustckinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL3 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL4 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunctuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunctuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH3 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunctuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH4 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, crtumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansinc, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL3 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL4 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH3 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezckimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH4 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VL4 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VH4 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VL4 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptide complexes provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptide complexes provided herein comprise a VH4 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans. Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium. Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptide complexes provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL3 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL4 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH3 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH4 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL2 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL3 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL4 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH3 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH4 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOS: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOS: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VL4 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VH4 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VL4 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptide complexes provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptide complexes provided herein comprise a VH4 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptide complexes provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL3 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL4 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, cleanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH3 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH4 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL3 comprising a VL of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL4 comprising a VL of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH3 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, clezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH4 comprising a VH of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VL4 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VH4 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VL4 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptide complexes provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptide complexes provided herein comprise a VH4 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, an antigen binding polypeptide complex provided herein comprises a first polypeptide, and a second polypeptide; wherein the first polypeptide has a structure represented by VL1-L1-VL2-L2-CH1 or VL2-L1-VL1-L2-CH1; and wherein the second polypeptide has a structure represented by VH1-L3-VH2-L4-CL or VH2-L3-VH1-L4-CL.

In some aspects, an antigen binding polypeptide complex provided herein comprises a first polypeptide, and a second polypeptide; wherein the first polypeptide has a structure represented by VL1-VL2-CH1-CH2-CH3 or VL2-VL1-CH1-CH2-CH3; and wherein the second polypeptide has a structure represented by VH1-VH2-CL-CH2-CH3 or VH2-VH1-CL-CH2-CH3.

In some aspects, an antigen binding polypeptide complex provided herein comprises a first polypeptide, and a second polypeptide; wherein the first polypeptide has a structure represented by VL1-L1-VL2-L2-CH1-CH2-CH3 or VL2-L1-VL1-L2-CH1-CH2-CH3; and wherein the second polypeptide has a structure represented by VH1-L3-VH2-L4-CL-CH2-CH3 or VH2-L3-VH1-L4-CL-CH2-CH3.

In some aspects, an antigen binding polypeptide complex provided herein comprises a first polypeptide, and a second polypeptide; wherein the first polypeptide has a structure represented by VL1-VH1-CL or VH1-VL1-CL; and wherein the second polypeptide has a structure represented by VL2-VH2-CH1 or VH2-VL2-CH1.

In some aspects, an antigen binding polypeptide complex provided herein comprises a first polypeptide, and a second polypeptide; wherein the first polypeptide has a structure represented by VL1-L1-VH1-L2-CL or VH1-L1-VL1-L2-CL; and wherein the second polypeptide has a structure represented by VL2-L3-VH2-L4-CH1 or VH2-L3-VL2-L4-CH1.

In some aspects, an antigen binding polypeptide complex provided herein comprises a first polypeptide, and a second polypeptide; wherein the first polypeptide has a structure represented by VL1-VH1-CL-CH2-CH3 or VH1-VL1-CL-CH2-CH3; and wherein the second polypeptide has a structure represented by VL2-VH2-CH1-CH2-CH3 or VH2-VL2-CH1-CH2-CH3.

In some aspects, an antigen binding polypeptide complex provided herein comprises a first polypeptide, and a second polypeptide; wherein the first polypeptide has a structure represented by VL1-L1-VH1-L2-CL-CH2-CH3 or VH1-L1-VL1-L2-CL-CH2-CH3; and wherein the second polypeptide has a structure represented by VL2-L3-VH2-L4-CH1-CH2-CH3 or VH2-L3-VL2-L4-CH1-CH2-CH3. In some aspects, VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; and VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; CH1 is an immunoglobulin heavy chain constant region 1; CL is an immunoglobulin light chain constant region; and L1-L4 are optional amino acid linkers. In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the VL1 and the VL2 each comprise a CDR1, a CDR2, and a CDR3 that are the same. In some aspects, the VL1 and the VL2 each comprise a CDR1, a CDR2, and a CDR3 that are different.

In some aspects, the VL1 and the VL2 each comprise an amino acid sequence, wherein the amino acid sequence comprised in VL1 and the amino acid sequence comprised in VL2 are the same. In some aspects, the VL1 and the VL2 each comprise an amino acid sequence, wherein the amino acid sequence comprised in VL1 and the amino acid sequence comprised in VL2 are different.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90 granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40, OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLc, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptide complexes provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, cpitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexclizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, cnoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, cpitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae. Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium. Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptide complexes provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL2 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOS: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOS: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10) IP-10, repulsive guidance molecule A RGMA, IFN-γ, activated F9)/F10, calcitonin gene-related peptide (CGRP), angiopoietin 3, IFN receptor, IFN-γ, calcitonin, β-amyloid 1-40) and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptide complexes provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, clezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponczumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elevanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, clezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOS: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, an antigen binding polypeptide complex provided herein comprises a first polypeptide, and a second polypeptide; wherein the first polypeptide has a structure represented by VL1-VL2-VL3-CH1, VL1-VL3-VL2-CH1, VL2-VL1-VL3-CH1, VL2-VL3-VL1-CH1, VL3-VL1-VL2-CH1, or VL3-VL2-VL1-CH1; and wherein the second polypeptide has a structure represented by VH1-VH2-VH3-CL, VH1-VH3-VH2-CL, VH2-VH1-VH3-CL, VH2-VH3-VH1-CL, VH3-VH1-VH2-CL, or VH3-VH2-VH1-CL.

In some aspects, an antigen binding polypeptide complex provided herein comprises a first polypeptide, and a second polypeptide; wherein the first polypeptide has a structure represented by VL1-L1-VL2-L2-VL3-L3-CH1, VL1-L1-VL3-L2-VL2-L3-CH1, VL2-L1-VL1-L2-VL3-L3-CH1, VL2-L1-VL3-L2-VL1-L3-CH1, VL3-L1-VL1-L2-VL2-L3-CH1, or VL3-L1-VL2-L2-VL1-L3-CH1; and wherein the second polypeptide has a structure represented by VH1-L4-VH2-L5-VH3-L6-CL, VH1-L4-VH3-L5-VH2-L6-CL, VH2-L4-VH1-L5-VH3-L6-CL, VH2-L4-VH3-L5-VH1-L6-CL, VH3-L4-VH1-L5-VH2-L6-CL, or VH3-L4-VH2-L5-VH1-L6-CL.

In some aspects, an antigen binding polypeptide complex provided herein comprises a first polypeptide, and a second polypeptide; wherein the first polypeptide has a structure represented by VL1-VL2-VL3-CH1-CH2-CH3, VL1-VL3-VL2-CH1-CH2-CH3, VL2-VL1-VL3-CH1-CH2-CH3, VL2-VL3-VL1-CH1-CH2-CH3, VL3-VL1-VL2-CH1-CH2-CH3, or VL3-VL2-VL1-CH1-CH2-CH3; and wherein the second polypeptide has a structure represented by VH1-VH2-VH3-CL-CH2-CH3, VH1-VH3-VH2-CL-CH2-CH3, VH2-VH1-VH3-CL-CH2-CH3, VH2-VH3-VH1-CL-CH2-CH3, VH3-VH1-VH2-CL-CH2-CH3, or VH3-VH2-VH1-CL-CH2-CH3.

In some aspects, an antigen binding polypeptide complex provided herein comprises a first polypeptide, and a second polypeptide; wherein the first polypeptide has a structure represented by VL1-L1-VL2-L2-VL3-L3-CH1-CH2-CH3, VL1-L1-VL3-L2-VL2-L3-CH1-CH2-CH3, VL2-L1-VL1- L2-VL3-L3-CH1-CH2-CH3, VL2-L1-VL3-L2-VL1-L3-CH1-CH2-CH3, VL3-L1-VL1-L2-VL2-L3-CH1- CH2-CH3, or VL3-L1-VL2-L2-VL1-L3-CH1-CH2-CH3; and wherein the second polypeptide has a structure represented by VH1-L1-VH2-L2-VH3-L3-CL-CH2-CH3, VH1-L1-VH3-L2-VH2-L3-CL-CH2-CH3, VH2-L1-VH1-L2-VH3-L3-CL-CH2-CH3, VH2-L1-VH3-L2-VH1-L3-CL-CH2-CH3, VH3-L1-VH1-L2-VH2-L3-CL-CH2-CH3, or VH3-L1-VH2-L2-VH1-L3-CL-CH2-CH3.

In some aspects, VL1 is a first immunoglobulin light chain variable region that specifically binds to (i) a tumor-associated antigen (TAA), (ii) an infectious disease antigen that is not a sarbecovirus antigen, or (iii) an other-pathology antigen; VL2 is a second immunoglobulin light chain variable region that specifically binds to (i) a tumor-associated antigen (TAA), (ii) an infectious disease antigen that is not a sarbecovirus antigen, or (iii) an other-pathology antigen; VL3 is a third immunoglobulin light chain variable region that specifically binds to (i) a tumor-associated antigen (TAA), (ii) an infectious disease antigen that is not a sarbecovirus antigen, or (iii) an other-pathology antigen; VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; VH3 is a third immunoglobulin heavy chain variable region that specifically binds to (i) a tumor-associated antigen (TAA), (ii) an infectious disease antigen that is not a sarbecovirus antigen, or (iii) an other-pathology antigen; CH1 is an immunoglobulin heavy chain constant region 1; CL is an immunoglobulin light chain constant region; and L1-L6 are optional amino acid linkers. In any aspect shown herein as a structure from amino terminus to carboxy terminus, and with binding pairs selected from VL and/or VH or other structural regions such as CH1, CL, CH2, CH3, when shown without a specific linker such as L1, L2, etc., such linkers are optionally present in such structures between each designated subsequence VL, VH, etc.

In some aspects, the VL1 and the VL2 each comprise a CDR1, a CDR2, and a CDR3 that are the same. In some aspects, the VL1 and the VL2 each comprise a CDR1, a CDR2, and a CDR3 that are different. In some aspects, the VL1 and the VL3 each comprise a CDR1, a CDR2, and a CDR3 that are the same. In some aspects, the VL1 and the VL3 each comprise a CDR1, a CDR2, and a CDR3 that are different. In some aspects, the VL2 and the VL3 each comprise a CDR1, a CDR2, and a CDR3 that are the same. In some aspects, the VL2 and the VL3 each comprise a CDR1, a CDR2, and a CDR3 that are different.

In some aspects, the VL1 and the VL2 each comprise an amino acid sequence, wherein the amino acid sequence comprised in VL1 and the amino acid sequence comprised in VL2 are the same. In some aspects, the VL1 and the VL2 each comprise an amino acid sequence, wherein the amino acid sequence comprised in VL1 and the amino acid sequence comprised in VL2 are different. In some aspects, the VL1 and the VL3 each comprise an amino acid sequence, wherein the amino acid sequence comprised in VL1 and the amino acid sequence comprised in VL3 are the same. In some aspects, the VL1 and the VL3 each comprise an amino acid sequence, wherein the amino acid sequence comprised in VL1 and the amino acid sequence comprised in VL3 are different. In some aspects, the VL2 and the VL3 each comprise an amino acid sequence, wherein the amino acid sequence comprised in VL2 and the amino acid sequence comprised in VL3 are the same. In some aspects, the VL2 and the VL3 each comprise an amino acid sequence, wherein the amino acid sequence comprised in VL2 and the amino acid sequence comprised in VL3 are different.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, ILSR, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70)-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40), OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialic acid, PROM1, prostase, prostein, PRSS21, PSCA, PSMA, PTK7, RAGE-1, RANKL. Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRPI glycoprotein 75, UPK2, VAP-1, VEGF, VEGFA, VEGFR-1, VEGFR2, vimentin, VISTA, Vstm3, WT1, WUCAM, and XAGE1.

In some aspects, the antigen binding polypeptide complexes provided herein comprise a VL1 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL2 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobclimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL3 comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH3 comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL1 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL2 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zcripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, cpitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL3 comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulcsomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH1 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozclimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH2 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, bclimumab, tabalumab, tibulizumab, belantamab mafodotin, teclistamab, teclistamab, detumomab, nacolomab tafenatox, eculizumab, lendalizumab, pexelizumab, pozclimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zubcritamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VH3 comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the antigen binding polypeptide complexes provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein, F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2, E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine, Hsp90, CCR5, lipoteichoic acid, clumping factor A).

In some aspects, the antigen binding polypeptide complexes provided herein comprise a VL1 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL2 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL3 comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH3 comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL1 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL2 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL3 comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH1 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH2 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VH3 comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOS: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the antigen binding polypeptide complexes provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor. Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the antigen binding polypeptide complexes provided herein comprise a VL1 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL2 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL3 comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, clezanumab, emapalumab, emicizumab, eptinezumab, erenumab, evinacumab, faralimomab, fasinumab, fontolizumab, fremanezumab, frunevetmab, fulranumab, galcanczumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH1 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH2 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH3 comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, cleanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL1 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL2 comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL3 comprising a VL of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanezumab, stamulumab, suptavumab, sutimlimab, tanezumab, crovalimab, and lokivetmab.

In some aspects, the antigen binding polypeptide complexes provided herein comprise a VH1 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanezumab, stamulumab, suptavumab, sutimlimab, tanezumab, crovalimab, and lokivetmab.

In some aspects, the antigen binding polypeptide complexes provided herein comprise a VH2 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VH3 comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the antigen binding polypeptide complexes provided herein comprise a VL1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VL2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VL3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the antigen binding polypeptide complexes provided herein comprise a VH1 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VH2 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VH3 comprising (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the antigen binding polypeptide complexes provided herein comprise a VL1 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VL2 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VL3 comprising an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the antigen binding polypeptide complexes provided herein comprise a VH1 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptide complexes provided herein comprise a VH2 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptide complexes provided herein comprise a VH3 comprising an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971. In some aspects, the first polypeptide comprises a structure represented by VL1-CL-VL2-VH2-VH1-CH1, VL1-L1-CL-L2-VL2-L3-VH2-L4-VH1-L5-CH1, VL1-CL-L1-VL2-L2-VH2-L3-VH1-CH1, VL1-CL-VL2-VH2-VH1-CH1-CH2-CH3, VL1-L1-CL-L2-VL2-L3-VH2-L4-VH1-L5-CH1-CH2-CH3, or VL1-CL-L1-VL2-L2-VH2-L3- VH1-CH1-CH2-CH3, and the second polypeptide comprises a structure represented by VL3-CL-VL4-VH4-VH3-CH1, VL3-L6-CL-L7-VL4-L8-VH4-L9-VH3-L10-CH1, VL3-CL-L4-VL4-L5-VH4-L6-VH3-CH1, VL3-CL-VL4-VH4-VH3-CH1-CH2-CH3, VL3-L6-CL-L7-VL4-L8-VH4-L9-VH3-L10-CH1-CH2-CH3, or VL3-CL-L4-VL4-L5-VH4-L6-VH3-CH1-CH2-CH3,
wherein:
(a)
(i)
VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezckimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zcripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulcsomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zcripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL3 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL4 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH3 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b)
(i)
VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL3 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL4 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VH1 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH2 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH3 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; and/or
VH4 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008; or
(c)
(i)
VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanezumab, stamulumab, suptavumab, sutimlimab, tanezumab, crovalimab, and lokivetmab;
VH3 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH4 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanczumab, stamulumab, suptavumab, sutimlimab, tanezumab, crovalimab, and lokivetmab; or
(ii)
VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a VH of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanezumab, stamulumab, suptavumab, sutimlimab, tanezumab, crovalimab, and lokivetmab;
VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH4 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanezumab, stamulumab, suptavumab, sutimlimab, tanezumab, crovalimab, and lokivetmab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL4 comprises an amino acid sequence that is at least 65%, at least 70%, at least 75%, at least 80%, at least 85%, at least 90%, at least 95%, at least 99%, or 100% identical to any one of SEQ ID NOS: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH3 comprises an amino acid sequence that is at least 65%, at least 70%, at least 75%, at least 80%, at least 85%, at least 90%, at least 95%, at least 99%, or 100% identical to any one of SEQ ID NOS: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOS: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOS: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the first polypeptide comprises a structure represented by VL1-VH1-VL2-CL-VH2-CH1, VL1-L1-VH1-L2-VL2-L3-CL-L4-VH2-L5-CH1, VL1-VH1-VL2-CL-VH2-CH1-CH2-CH3, or VL1-L1-VH1-L2-VL2-L3-CL-L4-VH2-L5-CH1-CH2-CH3 and the second polypeptide comprises a structure represented by VL3-VH3-VL4-CL-VH4-CH1, VL3-L6-VH3-L7-VL4-L8-CL-L9-VH4-L10-CH1, VL3-VH3-VL4-CL-VH4-CH1-CH2-CH3, or VL3-L6-VH3-L7-VL4-L8-CL-L9-VH4-L10-CH1-CH2-CH3,
wherein:
(a)
(i)
VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezckimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zubcritamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fczakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozclimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zubcritamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexclizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, gusclkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zcripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, crtumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansinc, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL3 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL4 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH3 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b)
(i)
VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOS: 1794, 1800, 1806, 1812, 1819, 1826, 1980, 1988, 1996, and 2004;
VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL3 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL4 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VH1 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008;
VH2 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH3 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; and/or
VH4 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; or
(c)
(i)
VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, devamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, devamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH4 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, clezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(ii)
VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanezumab, stamulumab, suptavumab, sutimlimab, tanezumab, crovalimab, and lokivetmab;
VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH4 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL4 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VH1 comprises an amino acid sequence that is at least 65%, at least 70%, at least 75%, at least 80%, at least 85%, at least 90%, at least 95%, at least 99%, or 100% identical to any one of SEQ ID NOS: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH3 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635.636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOS: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the first polypeptide comprises a structure represented by VL1-CL-VH1-CH1-VL2-VH2, VL1-L1-CL-L2-VH1-L3-CH1-L4-VL2-L5-VH2, VL1-CL-VH1-CH1-VL2-VH2-CH2-CH3, or VL1-L1-CL-L2-VH1-L3-CH1-L4-VL2-L5-VH2-CH2-CH3 and the second polypeptide comprises a structure represented by VL3-CL-VH3-CH1-VL4-VH4, VL3-L6-CL-L7-VH3-L8-CH1-L9-VL4-L10-VH4, VL3-CL-VH3-CH1-VL4-VH4-CH2-CH3, or VL3-L6-CL-L7-VH3-L8-CH1-L9-VL4-L10-VH4-CH2-CH3,
wherein:
(a)
(i)
VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezckimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulcsomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zcripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, bclimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulcsomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zcripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL3 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL4 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH3 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b)
(i)
VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL2 comprises an amino acid sequence that is at least 65%, at least 70%, at least 75%, at least 80%, at least 85%, at least 90%, at least 95%, at least 99%, or 100% identical to any one of SEQ ID NOS: 1794, 1800, 1806, 1812, 1819, 1826, 1980, 1988, 1996, and 2004;
VL3 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL4 comprises an amino acid sequence that is at least 65%, at least 70%, at least 75%, at least 80%, at least 85%, at least 90%, at least 95%, at least 99%, or 100% identical to any one of SEQ ID NOS: 1794, 1800, 1806, 1812, 1819, 1826, 1980, 1988, 1996, and 2004;
VH1 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH2 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008;
VH3 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; and/or
VH4 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; or
(c)
(i)
VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD00) 1, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanezumab, stamulumab, suptavumab, sutimlimab, tanezumab, crovalimab, and lokivetmab;
VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapincuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanezumab, stamulumab, suptavumab, sutimlimab, tanezumab, crovalimab, and lokivetmab; and/or
VH4 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(ii)
VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanczumab, stamulumab, suptavumab, sutimlimab, tanezumab, crovalimab, and lokivetmab;
VL4 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, sctrusumab, SHP647, solanezumab, stamulumab, suptavumab, sutimlimab, tanezumab, crovalimab, and lokivetmab; and/or
VH4 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL4 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH3 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and/or
VH4 comprises an amino acid sequence that is at least 65%, at least 70%, at least 75%, at least 80%, at least 85%, at least 90%, at least 95%, at least 99%, or 100% identical to any one of SEQ ID NOS: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOS: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOS: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the first polypeptide comprises a structure represented by VL1-VL2-VH2-VH1-CL-CH1, VL1-L1-VL2-L2-VH2-L3-VH1-L4-CL-L5-CH1, VL1-VL2-VH2-VH1-CL-CH1-CH2-CH3, or VL1-L1-VL2-L2-VH2-L3-VH1-L4-CL-L5-CH1-CH2-CH3 and the second polypeptide comprises a structure represented by VL3-VL4-VH4-VH3-CL-CH1, VL3-L6-VL4-L7-VH4-L8-VH3-L9-CL-L10-CH1, VL3-VL4-VH4-VH3-CL-CH1-CH2-CH3, or VL3-L6-VL4-L7-VH4-L8-VH3-L9-CL-L10-CH1-CH2-CH3,
wherein:
(a)
(i)
VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, crtumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anctumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansinc, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunctuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibctuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, crlizumab, lirilumab, relatlimab, cBR96-doxorubicinimmunoconjugate, odulimomab, efalizumab, ladiratuzumab vedotin, simtuzumab, samrotamab vedotin, asclizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsctuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezckimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermckimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbctuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, scribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL3 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL4 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH3 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b)
(i)
VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL3 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL4 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VH1 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH2 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH3 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; and/or
VH4 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008; or
(c)
(i)
VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, devamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH4 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(ii)
VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH4 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL4 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH3 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the first polypeptide comprises a structure represented by VL1-VH1-VL2-CL-VH2-CH1, VL1-L1-VH1-L2-VL2-L3-CL-L4-VH2-L5-CH1, VL1-VH1-VL2-CL-VH2-CH1-CH2-CH3, or VL1-L1-VH1-L2-VL2-L3-CL-L4-VH2-L5-CH1-CH2-CH3 and the second polypeptide comprises a structure represented by VL3-CL-VH3-CH1-VL4-VH4, VL3-L6-CL-L7-VH3-L8-CH1-L9-VL4-L10-VH4, VL3-CL-VH3-CH1-VL4-VH4-CH2-CH3, or VL3-L6-CL-L7-VH3-L8-CH1-L9-VL4-L10-VH4-CH2-CH3,
wherein:
(a)
(i)
VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL3 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL4 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH3 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b)
(i)
VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL3 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL4 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VH1 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH2 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH3 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; and/or
VH4 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; or
(c)
(i)
VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH4 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(ii)
VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH4 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL4 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH3 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the first polypeptide comprises a structure represented by VL1-CL-VH1-CH1-VL2-VH2, VL1-L1-CL-L2-VH1-L3-CH1-L4-VL2-L5-VH2, VL1-CL-VH1-CH1-VL2-VH2-CH2-CH3, or VL1-L1-CL-L2-VH1-L3-CH1-L4-VL2-L5-VH2-CH2-CH3 and the second polypeptide comprises a structure represented by VL3-VH3-VL4-CL-VH4-CH1, VL3-L6-VH3-L7-VL4-L8-CL-L9-VH4-L10-CH1, VL3-VH3-VL4-CL-VH4-CH1-CH2-CH3, or VL3-L6-VH3-L7-VL4-L8-CL-L9-VH4-L10-CH1-CH2-CH3,
wherein:
(a)
(i)
VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexclimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL4 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH3 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH4 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL3 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL4 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH3 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b)
(i)
VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL4 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH3 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH4 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL3 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL4 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VH1 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH2 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH3 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; and/or
VH4 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; or
(c)
(i)
VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH4 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(ii)
VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL4 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH3 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or VH4 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and/or
VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL4 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH3 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and/or
VH4 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the first polypeptide comprises a structure represented by VL1-VL2-CH1; VL1-L1-VL2-L2-CH1; VL1-VL2-CH1-CH2-CH3; or VL1-L1-VL2-L2-CH1-CH2-CH3 and the second polypeptide comprises a structure represented by VH1-VH2-CL; VH1-L3-VH2-L4-CL; VH1-VH2-CL-CH2-CH3; or VH1-L3-VH2-L4-CL-CH2-CH3,
wherein:
(a)
(i)
VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and/or
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and/or
VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b)
(i)
VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and/or
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VH1 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; and/or
VH2 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; or
(c)
(i)
VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(ii)
VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and/or
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and/or
VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the first polypeptide comprises a structure represented by VL1-VL2-VL3-CH1; VL1-L1-VL2-L2-VL3-L3-CH1; VL1-VL2-VL3-CH1-CH2-CH3; or VL1-L1-VL2-L2-VL3-L3-CH1-CH2-CH3 and the second polypeptide comprises a structure represented by VH1-VH2-VH3-CL; VH1-L4-VH2-L5-VH3-L6-CL; or VH1-L4-VH2-L5-VH3-L6-CL-CH2-CH3,
wherein:
(a)
(i)
VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH3 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL3 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH3 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and/or
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL3 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792;
VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and/or
VH3 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b)
(i)
VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH3 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL3 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH3 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and/or
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL3 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VH1 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008;
VH2 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; and/or
VH3 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; or
(c)
(i)
VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH3 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(ii)
VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL3 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH3 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973;
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and/or
VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974;
VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and/or
VH3 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the first polypeptide comprises a structure represented by VL1-VH1-CL, VL1-L1-VH1-L2-CL; VL1-VH1-CL-CH2-CH3; VL1-L1-VH1-L2-CL-CH2-CH3 and the second polypeptide comprises a structure represented by VL2-VH2-CH1; VL2-L3-VH2-L4-CH1; VL2-VH2-CH1-CH2-CH3; VL2-L3-VH2-L4-CH1-CH2-CH3,
wherein:
(a)
(i)
VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab;
VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; and/or
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788;
VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; and/or
VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b)
(i)
VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab;
VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; and/or
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004;
VH1 comprises an amino acid sequence that is at least 65%, at least 70%, at least 75%, at least 80%, at least 85%, at least 90%, at least 95%, at least 99%, or 100% identical to any one of SEQ ID NOS 1797, 1804, 1809, 1816, 1823, 1830, 1984, 1992, 2000, and 2008; and/or
VH2 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008; or
(c)
(i)
VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD00) 1, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or
VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(ii)
VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab;
VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(iii)
VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970;
VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; and/or
VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973; or
(iv)
VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971;
VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974; and/or
VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635.636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, an antigen binding polypeptide complex provided herein comprises a first polypeptide, and a second polypeptide; wherein the first polypeptide has a structure represented by:
VL1-L1-VL2-L2-VH2-L3-VH1-L4-Fc;
VL1-L1-VH2-L2-VL2-L3-VH1-L4-Fc;
VH1-L1-VH2-L2-VL2-L3-VL1-L4-Fc; or
VH1-L1-VL2-L2-VH2-L3-VL1-L4-Fc;
   and wherein the second polypeptide has a structure represented by:
VL3-L5-VL4-L6-VH4-L7-VH3-L8-Fc;
VL3-L5-VH4-L6-VL4-L7-VH3-L8-Fc;
VH3-L5-VH4-L6-VL4-L7-VL3-L8-Fc; or
VH3-L5-VL4-L6-VH4-L7-VL3-L8-Fc;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VL3 is a third immunoglobulin light chain variable region that specifically binds to (i) a tumor-associated antigen (TAA), (ii) an infectious disease antigen that is not a sarbecovirus antigen, or (iii) an other-pathology antigen;
VL4 is a fourth immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
VH3 is a third immunoglobulin heavy chain variable region that specifically binds to (i) a tumor-associated antigen (TAA), (ii) an infectious disease antigen that is not a sarbecovirus antigen, or (iii) an other-pathology antigen;
VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen;
L1-L8 are optional amino acid linkers; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In some aspects, an antigen binding polypeptide complex provided herein comprises a first polypeptide, and a second polypeptide; wherein the first polypeptide has a structure represented by:
VL1-L1-VL2-L2-VH2-L3-VH1-L4-CH1-L5-CL-L6-Fc;
VL1-L1-VH2-L2-VL2-L3-VH1-L4-CH1-L5-CL-L6-Fc;
VH1-L1-VH2-L2-VL2-L3-VL1-L4-CH1-L5-CL-L6-Fc;
VH1-L1-VL2-L2-VH2-L3-VL1-L4-CH1-L5-CL-L6-Fc;
VL1-L1-VL2-L2-VH2-L3-VH1-L4-CL-L5-CH1-L6-Fc;
VL1-L1-VH2-L2-VL2-L3-VH1-L4-CL-L5-CH1-L6-Fc;
VH1-L1-VH2-L2-VL2-L3-VL1-L4-CL-L5-CH1-L6-Fc; or
VH1-L1-VL2-L2-VH2-L3-VL1-L4-CL-L5-CH1-L6-Fc;
   and wherein the second polypeptide has a structure represented by:
VL3-L7-VL4-L8-VH4-L9-VH3-L10-CH1-L11-CL-L12-Fc;
VL3-L7-VH4-L8-VL4-L9-VH3-L10-CH1-L11-CL-L12-Fc;
VH3-L7-VH4-L8-VL4-L9-VL3-L10-CH1-L11-CL-L12-Fc;
VH3-L7-VL4-L8-VH4-L9-VL3-L10-CH1-L11-CL-L12-Fc;
VL3-L7-VL4-L8-VH4-L9-VH3-L10-CL-L11-CH1-L12-Fc;
VL3-L7-VH4-L8-VL4-L9-VH3-L10-CL-L11-CH1-L12-Fc;
VH3-L7-VH4-L8-VL4-L9-VL3-L10-CL-L11-CH1-L12-Fc; or
VH3-L7-VL4-L8-VH4-L9-VL3-L10-CL-L11-CH1-L12-Fc;
wherein:
VL1 is a first immunoglobulin light chain variable region that specifically binds to a (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VL3 is a third immunoglobulin light chain variable region that specifically binds to (i) a tumor-associated antigen (TAA), (ii) an infectious disease antigen that is not a sarbecovirus antigen, or (iii) an other-pathology antigen;
VL4 is a fourth immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
VH3 is a third immunoglobulin heavy chain variable region that specifically binds to (i) a tumor-associated antigen (TAA), (ii) an infectious disease antigen that is not a sarbecovirus antigen, or (iii) an other-pathology antigen;
VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen;
L1-L12 are optional amino acid linkers;
CH1 is an immunoglobulin heavy chain constant region 1;
CL is an immunoglobulin light chain constant region; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, ILSR, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxylesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40, OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL, Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein, Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRP1 glycoprotein 75, UPK2, VAP-1, VEGF, VEGFA, VEGFR-1, VEGFR2, vimentin, VISTA, Vstm3, WT1, WUCAM, and XAGE1.

In some aspects, the VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL3 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL4 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH3 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH4 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL3 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL4 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH3 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH4 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the VL3 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the VL4 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the VH3 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the VH4 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein. F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine. Hsp90), CCR5, lipoteichoic acid, clumping factor A).

In some aspects, the VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL3 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL4 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH3 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH4 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL3 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL4 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH3 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH4 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the VL3 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the VL4 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the VH3 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the VH4 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9), IFN-α/β receptor, Rhesus factor, nerve growth factor (NGF), DPP4, fibrin II beta chain, ACVR2B, serum amyloid A protein SOST, FGF 23, VWF, tissue factor pathway inhibitor (TFPI), 1-40) and 1-42, selectin P, glucagon receptor (GCGR), amyloid P component, GDF-8, CXCL10) IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40) and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A. Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL3 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL4 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH3 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH4 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL3 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL4 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH3 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH4 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the VL3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the VL4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the VH3 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the VH4 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the VL3 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the VL4 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the VH3 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the VH4 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, an antigen binding polypeptide complex provided herein comprises two antigen binding polypeptides wherein at least one antigen binding polypeptide has a structure represented by:
VL1-L1-VL2-L2-VH2-L3-VH1-L4-Fc-L5-Fc;
VL1-L1-VH2-L2-VL2-L3-VH1-L4-Fc-L5-Fc;
VH1-L1-VH2-L2-VL2-L3-VL1-L4-Fc-L5-Fc; or
VH1-L1-VL2-L2-VH2-L3-VL1-L4-Fc-L5-Fc;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
L1-L5 are optional amino acid linkers; and
Fc is an immunoglobulin fragment crystallizable region comprising an immunoglobulin heavy chain constant region 2 (CH2), an immunoglobulin heavy chain constant region 3 (CH3), and optionally, an immunoglobulin hinge.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD4+v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90 granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40, OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL, Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein. Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRP1 glycoprotein 75, UPK2, VAP-1, VEGF, VEGFA, VEGFR-1, VEGFR2, vimentin, VISTA, Vstm3, WT1, WUCAM, and XAGE1.

In some aspects, the VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein. F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50) (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine. Hsp90, CCR5, lipoteichoic acid, clumping factor A).

In some aspects, the VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor, Rhesus factor, nerve growth factor (NGF), DPP4, fibrin II beta chain, ACVR2B, scrum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A, Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 and 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 and 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, one or both of the antigen binding polypeptides comprised in the antigen binding polypeptide complexes disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, one or both of the antigen binding polypeptides comprised in the antigen binding polypeptide complexes disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, one or both of the antigen binding polypeptides comprised in the antigen binding polypeptide complexes provided herein further comprise an Fc region. In some aspects, one or both of the antigen binding polypeptides comprised in the antigen binding polypeptide complexes provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635.636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides complexes disclosed herein are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof, or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A, and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A, and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, one or both of the antigen binding polypeptides comprised in the antigen binding polypeptide complexes disclosed herein comprise one or more Fc effector function knockout mutation. In some aspects, one or both of the antigen binding polypeptides comprised in the antigen binding polypeptide complexes disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, one or both of the antigen binding polypeptides comprised in the antigen binding polypeptide complexes disclosed herein comprise a mutation at a glycosylation site. In some aspects, the mutation at a glycosylation site is a mutation at an N-glycosylation site. In some aspects, one or both of the antigen binding polypeptides comprised in the antigen binding polypeptide complexes disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62, or equivalent thereof, of the VH region or equivalent thereof. In some aspects, the mutation at an N-glycosylation site at amino acid N62 is N62Q or N62S, or equivalent thereof.

In some aspects, one or both of the antigen binding polypeptides comprised in the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the first polypeptide and the second polypeptide comprise a same amino acid sequence.

In some aspects, the first polypeptide and the second polypeptide comprise a different amino acid sequence.

In some aspects, the first polypeptide and the second polypeptide are encoded by the same nucleotide sequence.

In some aspects, the first polypeptide and the second polypeptide are encoded by a different nucleotide sequence.

In some aspects, the antigen binding polypeptides or the antigen binding polypeptide complexes disclosed herein, comprise one or more optional linkers. The one or more optional linkers are indicated herein as Ln, wherein n is an integer, and the integer next to each L indicates each L comprised in the antigen binding polypeptides or the antigen binding polypeptide complexes disclosed herein. For example, if an antigen binding polypeptide disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3.

In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptides or the antigen binding polypeptide complexes disclosed herein, comprise one or more CL, as indicated in the formulas representing the antigen binding polypeptides or the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides or the antigen binding polypeptide complexes disclosed herein, comprise one or more CH1, as indicated in the formulas representing the antigen binding polypeptides or the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides or the antigen binding polypeptide complexes disclosed herein, comprise one or more hinge regions, wherein a hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074.

In some aspects, the antigen binding polypeptides or the antigen binding polypeptide complexes disclosed herein, comprise one or more Fc region. In some aspects, the Fc region is at the carboxy-terminus of the antigen binding polypeptides disclosed herein. In some aspects, the Fc region is at the carboxy-terminus of at least one of the antigen binding polypeptides comprised in the antigen binding polypeptide complexes disclosed herein. In some aspects, the Fc region comprises at least one knob-into-hole modification or Fc effector function knockout mutation. In some aspects, the Fc region comprises at least one knob-into-hole modification or Fc effector function knockout mutation. In some aspects, the Fc region comprises an immunoglobulin heavy chain constant region 2 (CH2) and/or an immunoglobulin heavy chain constant region 3 (CH3), as indicated in the formulas representing the antigen binding polypeptides or the antigen binding polypeptide complexes disclosed herein. In some aspects, the Fc region further comprises an immunoglobulin hinge (i.e., a hinge region). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise one or more of the following amino acid substitutions:
M428L and N434A (LA), or equivalent thereof;
M428L and N434S (LS), or equivalent thereof;
L234F and L235E, or equivalent thereof;
L234F, L235E, and P331S (TM), or equivalent thereof; and/or
M252Y, S254T, and T256E (YTE), or equivalent thereof;
based on the EU numbering scheme.

In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence at least 65%, at least 70%, at least 75%, at least 80%, at least 85%, at least 90%, at least 95%, at least 99%, or 100% identical to any one of SEQ ID NOs: 376-380.

In some aspects, the Fc region comprises at least one knob-into-hole modification or Fc effector function knockout mutation. In some aspects, the antigen binding polypeptide or the antigen binding polypeptide complex disclosed herein is an IgG1 or IgG4 antibody or antigen binding fragment thereof and the knob-into-hole modification comprises:
(i) knob substitutions of S354C and T366W, or equivalent thereof;
(ii) hole substitutions Y349C, T366S, L368A and Y407V, or equivalent thereof;
(iii) a combination thereof;
based on the EU numbering scheme.

In some aspects, the knob-into-hole modification comprises one or more of knob substitutions of S354C and T366W, or equivalent thereof, and/or one or more of hole substitutions of Y349C, T366S, L368A, Y407V, or equivalent thereof; or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptide or the antigen binding polypeptide complex disclosed herein is an IgG1 or IgG4 antibody or antigen binding fragment thereof and the Fc effector function knockout mutation is L234A, L235A, P239A, or equivalent thereof, or a combination thereof, based on the EU numbering scheme.

In some aspects, an antigen binding polypeptide or antigen binding polypeptide complex disclosed herein comprises a VL comprising a CDR1, a CDR2, and a CDR3 of any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004.

In some aspects, an antigen binding polypeptide or antigen binding polypeptide complex disclosed herein comprises a VH comprising a CDR1, a CDR2, and a CDR3 of any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008.

In some aspects, an antigen binding polypeptide or antigen binding polypeptide complex disclosed herein comprises LCDR1 of any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, 1786, 1793, 1798, 1810, 1817, 1824, 1831, 1838, 1845, 1852, 1859, 1866, 1872, 1878, 1885, 1891, 1898, 1905, 1911, 1917, 1924, 1931, 1938, 1945, 1963, 1977, 1985, 1993, and 2001.

In some aspects, an antigen binding polypeptide or antigen binding polypeptide complex disclosed herein comprises LCDR2 of any one of SEQ ID NOs: 1200, 1212, 1680, 1690, 1698, 1709, 1728, 1739, 1744, 1753, 1770, 1777, 1978, 1986, 1994, and 2002; or any one of amino acid sequences AAS, APS, AT, ATS, AVS, DAS, DDG, DDS, DNN, DT, DTS, DVS, EAS, EDN, EVS, FTS, GAF, GAN, GAS, GAT, GIS, GKN, GNG, GNS, HTS, KAS, KVS, LAS, LGS, LVS, NAK, NTD, NTN, QMS, RMS, RTS, SAS, STS, TTS, WAS, YAS, YAT, YGS, YRS, and YTS.

In some aspects, an antigen binding polypeptide or antigen binding polypeptide complex disclosed herein comprises LCDR3 of any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, 1799, 1805, 1811, 1818, 1825, 1832, 1839, 1846, 1853, 1860, 1867, 1873, 1879, 1886, 1892, 1899, 1906, 1912, 1918, 1925, 1932, 1939, 1946, 1951, 1957, 1964, 1970, 1976, 1979, 1987, 1995, and 2003.

In some aspects, an antigen binding polypeptide or antigen binding polypeptide complex disclosed herein comprises HCDR1 of any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, 1789, 1795, 1801, 1813, 1820, 1827, 1834, 1841, 1848, 1855, 1862, 1881, 1894, 1901, 1920, 1927, 1934, 1941, 1953, 1959, 1966, 1981, 1989, 1997, and 2005.

In some aspects, an antigen binding polypeptide or antigen binding polypeptide complex disclosed herein comprises HCDR2 of any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, 1790, 1802, 1807, 1814, 1821, 1828, 1835, 1842, 1849, 1856, 1863, 1869, 1875, 1882, 1888, 1895, 1902, 1908, 1914, 1921, 1928, 1935, 1942, 1948, 1954, 1960, 1967, 1972, 1982, 1990, 1998, and 2006.

In some aspects, an antigen binding polypeptide or antigen binding polypeptide complex disclosed herein comprises HCDR3 of any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, 1796, 1803, 1808, 1815, 1822, 1829, 1836, 1843, 1850, 1857, 1864, 1870, 1876, 1883, 1889, 1896, 1903, 1909, 1915, 1922, 1929, 1936, 1943, 1949, 1955, 1961, 1968, 1973, 1975, 1983, 1991, 1999, and 2007.

In some aspects, an antigen binding polypeptide or antigen binding polypeptide complex disclosed herein comprises a VL of any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, 1788, 1794, 1800, 1806, 1812, 1819, 1826, 1833, 1840, 1847, 1854, 1861, 1868, 1874, 1880, 1887, 1893, 1900, 1907, 1913, 1919, 1926, 1933, 1940, 1947, 1952, 1958, 1965, 1971, 1980, 1988, 1996, and 2004.

In some aspects, an antigen binding polypeptide or antigen binding polypeptide complex disclosed herein comprises a VH of any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, 1792, 1797, 1804, 1809, 1816, 1823, 1830, 1837, 1844, 1851, 1858, 1865, 1871, 1877, 1884, 1890, 1897, 1904, 1910, 1916, 1923, 1930, 1937, 1944, 1950, 1956, 1962, 1969, 1974, 1984, 1992, 2000, and 2008.

In some aspects, an antigen binding polypeptide or antigen binding polypeptide complex disclosed herein comprise a detectable label. In some aspects, the detectable label is a radioactive label, chemiluminescent label, fluorescent label, enzyme, peptide tag, or a combination thereof. In some aspects, the peptide tag is a polyhistidine tag consisting of from about four to about 10 histidine residues. In some aspects, the poly histidine tag consists of about eight histidine residues.

In some aspects, an antigen binding polypeptide or antigen binding polypeptide complex disclosed herein is conjugated to an agent as an antibody-drug conjugate (ADC). In some aspects, the agent is a cytotoxic agent, immunomodulating agent, imaging agent, or therapeutic protein, or a combination thereof.

In some aspects, an antigen binding polypeptide or antigen binding polypeptide complex disclosed herein specifically binds to the (i) tumor-associated antigen (TAA), (ii) infectious disease antigen that is not a sarbecovirus antigen, or (iii) other-pathology antigen with an equilibrium dissociation constant (K_{D}) of from about 10 μM to about 1 pM.

In some aspects, an antigen binding polypeptide complex disclosed herein is bispecific, trispecific or tetraspecific and has greater binding affinity to the (i) tumor-associated antigen (TAA), (ii) infectious disease antigen that is not a sarbecovirus antigen, or (iii) other-pathology antigen than a monospecific antigen binding polypeptide complex that specifically binds to one of the same antigens as the bispecific, trispecific or tetraspecific antigen binding polypeptide complex.

In some aspects, an antigen binding polypeptide complex disclosed herein is bispecific, trispecific or tetraspecific and has greater binding affinity to the (i) tumor-associated antigen (TAA), (ii) infectious disease antigen that is not a sarbecovirus antigen, or (iii) other-pathology antigen than a mixture of monospecific antigen binding polypeptide complexes that specifically bind to the same antigens as the bispecific, trispecific or tetraspecific antigen binding polypeptide complex.

In some aspects, the antigen binding polypeptides and polypeptide complexes disclosed herein have broad reactivity against a viral antigen selected from the group consisting of influenza virus neuraminidase, influenza virus hemagglutinin, human respiratory syncytial virus (RSV)-viral proteins, RSV F glycoprotein, RSV G glycoprotein, herpes simplex virus (HSV) viral proteins, herpes simplex virus glycoproteins gB, gC, gD, and gE, chlamydia MOMP and PorB antigens, core protein, matrix protein or other protein of Dengue virus, measles virus hemagglutinin, herpes simplex virus type 2 glycoprotein gB, poliovirus 1 VP1, envelope glycoproteins of HIV 1, hepatitis B surface antigen, diptheria toxin. streptococcus 24M epitope, gonococcal pilin, pseudorabies virus g50) (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein. Serpulina hydodysenteriae protective antigen, bovine viral diarrhea glycoprotein 55. Newcastle disease virus hemagglutinin-neuraminidase, swine flu hemagglutinin, swine flu neuraminidase, foot and mouth disease virus, hog colera virus, swine influenza virus. African swine fever virus, Mycoplasma hyopneumoniae, infectious bovine rhinotracheitis virus, infectious bovine rhinotracheitis virus glycoprotein E, glycoprotein G, infectious laryngotracheitis virus, infectious laryngotracheitis virus glycoprotein G or glycoprotein I, a glycoprotein of La Crosse virus, neonatal calf diarrhoea virus. Venezuelan equine encephalomyelitis virus, punta toro virus, murine leukemia virus, mouse mammary tumor virus, hepatitis B virus core protein and hepatitis B virus surface antigen or a fragment or derivative thereof, antigen of equine influenza virus or equine herpes virus, including equine influenza virus type A/Alaska 91 neuraminidase, equine influenza virus typeA/Miami 63 neuraminidase, equine influenza virus type A/Kentucky 81 neuraminidase equine herpes virus type 1 glycoprotein B, and equine herpes virus type 1 glycoprotein D, antigen of bovine respiratory syncytial virus or bovine parainfluenza virus, bovine respiratory syncytial virus attachment protein (BRSV G), bovine respiratory syncytial virus fusion protein (BRSV F), bovine respiratory syncytial virus nucleocapsid protein (BRSVN), bovine parainfluenza virus type 3 fusion protein, bovine parainfluenza virus type 3 hemagglutinin neuraminidase, bovine E viral diarrhoea virus glycoprotein 48 and glycoprotein 53, glycoprotein E of Dengue virus, and glycoprotein E1E2 of human hepatitis C virus.

In some aspects, the antigen binding polypeptides and polypeptide complexes disclosed herein bind to at least one epitope on at least one antigen selected from the group consisting of influenza virus neuraminidase, influenza virus hemagglutinin, human respiratory syncytial virus (RSV)-viral proteins, RSV F glycoprotein, RSV G glycoprotein, herpes simplex virus (HSV) viral proteins, herpes simplex virus glycoproteins gB, gC, gD, and gE, chlamydia MOMP and PorB antigens, core protein, matrix protein or other protein of Dengue virus, measles virus hemagglutinin, herpes simplex virus type 2 glycoprotein gB, poliovirus 1 VP1, envelope glycoproteins of HIV 1, hepatitis B surface antigen, diptheria toxin, streptococcus 24M epitope, gonococcal pilin, pseudorabies virus g50) (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein. Serpulina hydodysenteriae protective antigen, bovine viral diarrhea glycoprotein 55. Newcastle disease virus hemagglutinin-neuraminidase, swine flu hemagglutinin, swine flu neuraminidase, foot and mouth disease virus, hog colera virus, swine influenza virus. African swine fever virus, Mycoplasma hyopneumoniae, infectious bovine rhinotracheitis virus, infectious bovine rhinotracheitis virus glycoprotein E, glycoprotein G, infectious laryngotracheitis virus, infectious laryngotracheitis virus glycoprotein G or glycoprotein I, a glycoprotein of La Crosse virus, neonatal calf diarrhoea virus. Venezuelan equine encephalomyelitis virus, punta toro virus, murine leukemia virus, mouse mammary tumor virus, hepatitis B virus core protein and hepatitis B virus surface antigen or a fragment or derivative thereof, antigen of equine influenza virus or equine herpes virus, including equine influenza virus type A/Alaska 91 neuraminidase, equine influenza virus typeA/Miami 63 neuraminidase, equine influenza virus type A/Kentucky 81 neuraminidase equine herpes virus type 1 glycoprotein B, and equine herpes virus type 1 glycoprotein D, antigen of bovine respiratory syncytial virus or bovine parainfluenza virus, bovine respiratory syncytial virus attachment protein (BRSV G), bovine respiratory syncytial virus fusion protein (BRSV F), bovine respiratory syncytial virus nucleocapsid protein (BRSVN), bovine parainfluenza virus type 3 fusion protein, bovine parainfluenza virus type 3 hemagglutinin neuraminidase, bovine E viral diarrhoea virus glycoprotein 48 and glycoprotein 53, glycoprotein E of Dengue virus, and glycoprotein E1E2 of human hepatitis C virus.

In some aspects, the antigen binding polypeptides and polypeptide complexes disclosed herein specifically bind to at least one epitope on at least one antigen selected from the group consisting of A2AR, APRIL, ATPDase, BAFF, BAFFR, BCMA. BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25 CCR3, CCR4, CD3, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, IL1A, IL1B, IL1F10, IL2, IL4, IL4Ra, IL5, ILSR, IL6, IL7, IL7Ra, IL8, IL9, IL9R, IL10, rhIL1O, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, and WUCAM.

In some aspects, the antigen binding polypeptides or the antigen binding polypeptide complexes disclosed herein comprise a VH comprising a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides or the antigen binding polypeptide complexes disclosed herein have an isoelectric point (pI) of from about 7 to about 9.

In some aspects, provided herein are antibodies or antigen binding fragments thereof comprising the antigen binding polypeptides or antigen binding polypeptide complexes disclosed herein. In some aspects, an antibody or antigen binding fragment thereof comprising the antigen binding polypeptides or antigen binding polypeptide complexes disclosed herein is IgG, IgM, IgE, IgA or IgD. In some aspects, the IgG is IgG1, IgG2, IgG3 or IgG4. In some aspects, an antibody or antigen binding fragment thereof comprising the antigen binding polypeptides or antigen binding polypeptide complexes disclosed herein is a Fab, scFab, Fab′, F(ab′)₂, Fv or scFv. In some aspects, an antibody or antigen binding fragment thereof comprising the antigen binding polypeptides or antigen binding polypeptide complexes disclosed herein is human or humanized.

In some aspects, provided herein are polynucleotides encoding the antigen binding polypeptides or antigen binding polypeptide complexes disclosed herein, or the antibodies or antigen binding fragments thereof comprising the antigen binding polypeptides or antigen binding polypeptide complexes disclosed herein.

In some aspects, provided herein are vectors comprising the polynucleotides disclosed herein. In some aspects, provided herein are host cells comprising the vectors disclosed herein.

In some aspects, provided herein are mRNAs encoding the antigen binding polypeptides, antigen binding polypeptide complexes, or the antibodies or antigen binding fragments thereof provided herein. In some aspects, the mRNAs provided herein comprise a 5′-cap and/or a poly(A) tail.

In some aspects, provided herein are lipid nanoparticles (LNP) comprising the mRNAs provided herein.

In some aspects, provided herein are chimeric antigen receptor (CAR) comprising the antigen binding polypeptide, antigen binding polypeptide complexes, or antibodies or antigen binding fragments thereof disclosed herein. In some aspects, provided herein are immune cells comprising the CARs disclosed herein.

In some aspects, provided herein are pharmaceutical compositions comprising the antigen binding polypeptides, the antigen binding polypeptide complexes, the antibodies or antigen binding fragments thereof, the polynucleotides, the vectors, the host cells, the mRNAs, the LNPs, the CARs, the immune cells disclosed herein, or any combination thereof. In some aspects, the pharmaceutical compositions disclosed herein further comprise a pharmaceutically acceptable carrier, an additional pharmaceutical agent, or a combination thereof.

In some aspects, provided herein are kits comprising the antigen binding polypeptides, the antigen binding polypeptide complexes, the antibodies or antigen binding fragments thereof, the polynucleotides, the vectors, the host cells, the mRNAs, the LNPs, the CARs, the immune cells, the pharmaceutical compositions disclosed herein, or any combination thereof.

In some aspects, provided herein are methods of preventing or treating (i) a cancer or a tumor, (ii) an infectious disease that is not a sarbecovirus infectious disease, or (iii) a pathology other than a cancer or a tumor or an infectious disease that is not a sarbecovirus infectious disease, comprising administering to the subject a therapeutically effective amount of the antigen binding polypeptides or antigen binding polypeptide complexes, the antibodies or antigen binding fragments thereof, the polynucleotides, the vectors, the host cells, the mRNAs, the LNPs, the CARs, the immune cells, or the pharmaceutical compositions disclosed herein, or any combination thereof.

In some aspects, provided herein are methods of diagnosing a subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptides or antigen binding polypeptide complexes or the antibodies or antigen binding fragments thereof disclosed herein; (b) detecting the presence or absence of a complex which contains the polypeptide or a fragment thereof, polypeptide complex or a fragment thereof, or antibody or antigen binding a fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is detected.

In some aspects, provided herein are methods of diagnosing a subject as not having or as not being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptides or antigen binding polypeptide complexes or the antibodies or antigen binding fragments thereof disclosed herein; (b) detecting the presence or absence of a complex which contains the polypeptide or a fragment thereof, polypeptide complex or a fragment thereof, or antibody or antigen bindinga fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as not having or as not being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is not detected.

In some aspects, the samples use in the methods provided herein are a nasal swab, tissue sample, saliva, plasma or blood. In some aspects, the methods provided herein comprise detecting the presence or absence of the virus complex comprises an enzyme linked immunosorbent assay (ELISA), immunospot assay, lateral flow assay, flow cytometry, immunohistochemistry or western blot.

In some aspects, provided herein are antibodies or an antigen binding fragments thereof, wherein the antibody or antigen binding fragment thereof binds to (i) a tumor-associated antigen (TAA), (ii) an infectious disease antigen that is not a sarbecovirus antigen, or (iii) an other-pathology antigen, and wherein the antibodies or an antigen binding fragments thereof comprise: (a) one or more immunoglobulin light chain constant domain (CL) and (b) one or more immunoglobulin heavy chain 1 constant domain (CH1); and wherein the antibodies or an antigen binding fragments thereof (i) have an isoelectric point (pI), and after administered to a subject has (ii) a higher peak serum levels, and (iii) a reduced clearance levels, as compared to a same antibody or antigen binding fragment thereof not comprising one or more immunoglobulin light chain constant domain (CL) and (b) one or more immunoglobulin heavy chain 1 constant domain (CH1).

It is to be understood that “a same antibody not comprising a CL and a CH1” refers to an antibody (or antigen binding fragment thereof) having a same amino acid sequence as the reference antibody (or antigen binding fragment thereof) exception made for the CL and the CH1 being absent from the “same antibody not comprising a CL and a CH1.” For example, a reference antibody (or antigen binding fragment thereof) (antibody R, or antigen binding fragment thereof) may comprise an amino acid sequence represented by the formula VL1-CL-VL2-VH2-VH1-CH1, “a same antibody not comprising a CL and a CH1” (antibody R′, or antigen binding fragment thereof) it is to be understood as comprising an amino acid sequence that is represented by the formula VL1-VL2-VH2-VH1, wherein the VL1, the VL2, the VH1, and the VH1 in the antibody R (or antigen binding fragment thereof) comprise an amino acid sequence identical to the amino acid sequence comprised in the VL1, the VL2, the VH1, and the VH1 comprised in the antibody R′ (or antigen binding fragment thereof).

In some aspects, an antibody or an antigen binding fragment thereof disclosed herein is an IgG, IgM, IgE, IgA or an IgD antibody or an antigen binding fragment thereof. In some aspects, an antibody or an antigen binding fragment thereof disclosed herein is IgG1, IgG2, IgG3 or IgG4. In some aspects, an antibody or an antigen binding fragment thereof disclosed herein is a Fab, scFab, Fab′, F(ab′)₂, Fv or scFv. In some aspects, an antibody or an antigen binding fragment thereof disclosed herein is human or humanized. In some aspects, an antibody or an antigen binding fragment thereof disclosed herein has an isoelectric point (pI) of from about 7 to about 9). In some aspects, an antibody or an antigen binding fragment thereof disclosed herein binds to the (i) tumor-associated antigen (TAA), (ii) infectious disease antigen that is not a sarbecovirus antigen, or (iii) other-pathology antigen with an equilibrium dissociation constant (K_{D})) of from about 10 μM to about 1 pM.

In some aspects, an antibody or an antigen binding fragment thereof disclosed herein is bispecific, trispecific or tetraspecific and has greater binding affinity to the (i) tumor-associated antigen (TAA), (ii) infectious disease antigen that is not a sarbecovirus antigen, or (iii) other-pathology antigen than a monospecific antibody or an antigen binding fragment thereof that specifically binds to one of the same antigens as the bispecific, trispecific or tetraspecific antibody or an antigen binding fragment thereof. In some aspects, an antibody or an antigen binding fragment thereof disclosed herein is bispecific, trispecific or tetraspecific and has greater binding affinity to the (i) tumor-associated antigen (TAA), (ii) infectious disease antigen that is not a sarbecovirus antigen, or (iii) other-pathology antigen than a mixture of monospecific antibodies or an antigen binding fragments thereof that specifically binds to one of the same antigens as the bispecific, trispecific or tetraspecific antibody or an antigen binding fragment thereof.

In some aspects, an antibody or an antigen binding fragment thereof disclosed herein comprises one or more variable heavy chain regions (VH) and at least one of the one or more VH has a mutation at an N-glycosylation site. In some aspects, the mutation is at amino acid N62 of the VH region or equivalent thereof. In some aspects, the N62 mutation is N62Q or N62S, or equivalent thereof.

In some aspects, an antibody or an antigen binding fragment thereof disclosed herein comprises at least one variable heavy chain region (VH) and/or at least one variable light chain region (VL).

In some aspects, an antibody or an antigen binding fragment thereof disclosed herein comprises:
(a)
(i)
a VL comprising a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
a VH comprising a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(ii)
VL comprising a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; and/or
a VH comprising a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab; or
(b)
(i)
a VL comprising a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
a VH comprising a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(ii)
a VL comprising a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; and/or
a VH comprising a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab; or
(c)
(i)
a VL comprising a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or a VH comprising a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; or
(ii)
a VL comprising a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab; and/or a VH comprising a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, an antibody or an antigen binding fragment thereof disclosed herein comprises at least one variable light chain region (VL). In some aspects, the at least one VL comprises:
(a)
(i) a complementarity determining region (CDR) 1 having an amino acid sequence at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786;
(ii) a CDR2 having an amino acid sequence at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS or an amino acid sequence at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; and
(iii) a CDR3 having an amino acid sequence at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976; or
(b)
(i) a CDR1 having an amino acid sequence at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001;
(ii) a CDR2 having an amino acid sequence at least 65%, at least 70%, at least 75%, at least 80%, at least 85%, at least 90%, at least 95%, at least 99%, or 100% identical to any one of amino acid sequences YTS, DTS, GAS, and AAS or an amino acid sequence at least 65%, at least 70%, at least 75%, at least 80%, at least 85%, at least 90%, at least 95%, at least 99%, or 100% identical to any one of SEQ ID NOs: 1978, 1986, 1994, and 2002; and
(iii) a CDR3 having an amino acid sequence at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003; or
(c)
(i) a CDR1 having an amino acid sequence at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963;
(ii) a CDR2 having an amino acid sequence at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and
(iii) a CDR3 having an amino acid sequence at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, an antibody or an antigen binding fragment thereof disclosed herein comprises at least one variable heavy chain region (VH). In some aspects, the at least one VH comprises:
(a)
(i) a CDR1 having an amino acid sequence at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789;
(ii) a CDR2 having an amino acid sequence at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and
(iii) a CDR3 having an amino acid sequence at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975; or
(b)
(i) a CDR1 having an amino acid sequence at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005;
(ii) a CDR2 having an amino acid sequence at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and
(iii) a CDR3 having an amino acid sequence at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007; or
(c)
(i) a CDR1 having an amino acid sequence at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966;
(ii) a CDR2 having an amino acid sequence at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and
(iii) a CDR3 having an amino acid sequence at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, an antibody or an antigen binding fragment thereof disclosed herein comprises at least one variable light chain region (VL). In some aspects, the at least one VL comprises an amino acid sequence:
(a) at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788; or
(b) at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004; or
(c) at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, an antibody or an antigen binding fragment thereof disclosed herein comprises at least one variable heavy chain region (VH). In some aspects, the at least one VH comprises an amino acid sequence:
(a) at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792; or
(b) at least 65%, at least 70%, at least 75%, at least 80%, at least 85%, at least 90%, at least 95%, at least 99%, or 100% identical to any one of SEQ ID NOs: 1797, 1804, 1809, 1816, 1823, 1830, 1984, 1992, 2000, and 2008; or
(c) at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

Molecular biology and recombinant DNA methods for making, screening and engineering an antigen binding polypeptide or antigen binding polypeptide complex (e.g., an antibody or antigen binding fragment thereof) containing such sequences are well known and described, for example, in Adair et al. Human Antibodies, 5(1-2):41-47, 1994; Kostelny et al., J Immunol. 148(5):1547-1553, 1992, Shiraiwa et al., Methods, 154:10-20, 2019; and Zola, “Monoclonal Antibodies: A Manual of Techniques.” 1987, 1st Ed., CRC Press; and Steinitz, Human Antibodies. 18(1-2):1-10, 2009.

As used herein, an antigen binding polypeptide or antigen binding polypeptide complex (e.g., an antibody or antigen binding fragment thereof), or region or domain thereof that “specifically binds” refers to its association with an epitope by its antigen binding domain, and that the binding entails some complementarity between the antigen binding domain and the epitope. Specific binding to an epitope occurs where there is binding to that epitope via its antigen binding domain more readily than there would be binding to a random, unrelated epitope.

As used herein, an “epitope” refers to a localized region of an antigen to which an antigen binding polypeptide or antigen binding polypeptide complex (e.g., antibody or antigen binding fragment thereof) can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy. X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). See, e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303; Meth Enzymol (1985) volumes 114 & 115, eds Wyckoff H W et al., U.S. Pub. No. 2004/0014194), Bricogne G (1993) Acta Crystallogr D Biol Crystallogr 49 (Pt 1): 37-60. Bricogne G (1997) Meth Enzymol 276A: 361-423, ed Carter C W, and Roversi et al., (2000) Acta Crystallogr D Biol Crystallogr 56 (Pt 10): 1316-1323 (X-ray diffraction crystallography studies); and Champe et al., (1995) J Biol Chem 270:1388-1394 and Cunningham B C & Wells J A (1989) Science 244:1081-1085 (mutagenesis mapping).

Specific binding can be represented by a “binding affinity.” Binding affinity refers to an intrinsic binding affinity which reflects a 1:1 interaction between members of a binding pair (e.g., an antigen binding polypeptide complex and an antigen). Binding affinity can be measured and/or expressed in several ways known in the art, including, but not limited to, equilibrium dissociation constant (K_{D}), K_{D} is calculated from the quotient of k_{off}/kₒₙ, where kₒₙ refers to the association rate constant of, e.g., an antigen binding polypeptide complex to an antigen, and k_{off} refers to the dissociation of, e.g., an antigen binding polypeptide complex from an antigen. The kₒₙ and k_{off} can be determined by techniques known to one of ordinary skill in the art, such as Octet®; BLI, BIAcore® or KinExA.

Accordingly, in some aspects, an antigen binding polypeptide complex provided herein is an antibody or antigen binding fragment thereof. In some aspects, an antigen binding polypeptide provided herein is part of an antibody or antigen binding fragment thereof.

In one aspect, the antibody or antigen binding fragment thereof specifically binds to an antigen with an equilibrium dissociation constant (K_{D}) of from about 10 μM to about 1 pM. In another aspect, the antibody or antigen binding fragment thereof is IgG, IgM, IgE, IgA or IgD. In another aspect, the IgG is IgG1, IgG2, IgG3 or IgG4. In another aspect, the antigen binding fragment is a Fab, scFab, Fab′, F(ab′)₂, Fv or scFv. In yet another aspect, the antibody or antigen binding fragment thereof is human or humanized.

In another aspect, an antigen binding polypeptide complex provided herein (e.g., an antibody or antigen binding fragment thereof) is trivalent or tetravalent.

In another aspect, an antigen binding polypeptide complex provided herein (e.g., an antibody or antigen binding fragment thereof) is monospecific, bispecific, trispecific, or tetraspecific.

In another aspect, an antigen binding polypeptide or antigen binding polypeptide complex, or antibody or antigen binding fragment thereof provided herein potently inhibits one or more pathogen.

In some aspects, provided herein are antibodies or antigen binding fragment thereof (e.g., monospecific or multispecific antibodies or antigen binding fragments thereof) comprising an antigen binding polypeptide having a structure represented by:
VL1-L1-VL2-L2-VH2-L3-VH1;
VL1-L1-VH2-L2-VL2-L3-VH1;
VH1-L1-VH2-L2-VL2-L3-VL1; or
VH1-L1-VL2-L2-VH2-L3-VL1;
wherein:
VL1 is a first immunoglobulin light chain variable region that specifically binds to a (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen; and
L1-L3 are optional amino acid linkers.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, ILSR, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxylesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40, OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL, Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein. Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRP1 glycoprotein 75, UPK2, VAP-1, VEGF, VEGFA, VEGFR-1, VEGFR2, vimentin, VISTA, Vstm3, WT1, WUCAM, and XAGE1.

In some aspects, the VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL2 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, blantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH2 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL2 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH2 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein. F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum, Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens, Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium. Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine. Hsp90, CCR5, lipoteichoic acid, clumping factor A).

In some aspects, the VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL2 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH2 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL2 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH2 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor, Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A. Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL2 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH2 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL2 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH2 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the VL2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the VH2 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the VL2 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the VH2 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides comprised in the antibodies or antigen binding fragments thereof (e.g., monospecific or multispecific antibodies or antigen binding fragments thereof) provided herein further comprise an Fc region. In some aspects, the antigen binding polypeptides comprised in the antibodies or antigen binding fragments thereof (e.g., monospecific or multispecific antibodies or antigen binding fragments thereof) provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof; or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides comprised in the antibodies or antigen binding fragments thereof (e.g., monospecific or multispecific antibodies or antigen binding fragments thereof) disclosed herein comprise one or more Fc effector function knockout mutation. In some aspects, the antigen binding polypeptides comprised in the antibodies or antigen binding fragments thereof (e.g., monospecific or multispecific antibodies or antigen binding fragments thereof) disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides comprised in the antibodies or antigen binding fragments thereof (e.g., monospecific or multispecific antibodies or antigen binding fragments thereof) disclosed herein comprise a mutation at a glycosylation site. In some aspects, the mutation at a glycosylation site is a mutation at an N-glycosylation site. In some aspects, the antigen binding polypeptides comprised in the antibodies or antigen binding fragments thereof (e.g., monospecific or multispecific antibodies or antigen binding fragments thereof) disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides comprised in the antibodies or antigen binding fragments thereof (e.g., monospecific or multispecific antibodies or antigen binding fragments thereof) disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between, L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptides comprised in the antibodies or antigen binding fragments thereof (e.g., monospecific or multispecific antibodies or antigen binding fragments thereof) disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides comprised in the antibodies or antigen binding fragments thereof (e.g., monospecific or multispecific antibodies or antigen binding fragments thereof) disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides comprised in the antibodies or antigen binding fragments thereof (e.g., monospecific or multispecific antibodies or antigen binding fragments thereof) provided herein further comprise an Fc region. In some aspects, the antigen binding polypeptides comprised in the antibodies or antigen binding fragments thereof (e.g., monospecific or multispecific antibodies or antigen binding fragments thereof) provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, provided herein are antibodies or antigen binding fragments thereof (e.g., monospecific or multispecific antibodies or antigen binding fragments thereof) comprising an antigen binding polypeptide having a structure represented by:
VL1-L1-VH1 or VH1-L1-VL1;
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen; and
L1 is an optional amino acid linker.

In any aspect shown herein as a structure from amino terminus to carboxy terminus, and with binding pairs selected from VL and/or VH or other structural regions such as CH2, CH3, when shown without a specific linker such as L1, L2, etc., such linkers are optionally present in such structures between each designated subsequence VL, VH, etc.

In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl, BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9, CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor, Fucosyl GM1, GD2, GD3, gelatinase B, Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, IL5R, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxy lesterase, ITGB4, ITK, KIR, KIR2D, KIT, LAG3, LAGE-1a, LAIR1, LAMP1, LCK, legumain, leptin. LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUC5AC, muthsp70-2, MYCN, NA17, NCA-90 granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40, OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL, Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein. Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRP1 glycoprotein 75, UPK2, VAP-1, VEGF, VEGFA, VEGFR-1, VEGFR2, vimentin, VISTA, Vstm3, WT1, WUCAM, and XAGE1.

In some aspects, the VL1 comprises a light chain complementarity determining region 1 (LCDR1), LCDR2, and/or LCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH1 comprises a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL1 comprises a VL of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VH1 comprises a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1096, 1103, 1110, 1120, 1127, 1134, 1141, 1148, 1155, 1162, 1169, 1174, 1181, 1188, 1199, 1211, 1221, 1228, 1235, 1247, 1254, 1261, 1266, 1271, 1278, 1285, 1292, 1299, 1306, 1313, 1319, 1326, 1339, 1352, 1359, 1366, 1373, 1382, 1394, 1401, 1408, 1414, 1421, 1428, 1441, 1448, 1455, 1461, 1468, 1475, 1488, 1495, 1508, 1519, 1526, 1531, 1538, 1544, 1556, 1568, 1574, 1581, 1594, 1601, 1608, 1615, 1622, 1629, 1636, 1643, 1650, 1657, 1672, 1679, 1689, 1697, 1708, 1715, 1718, 1723, 1727, 1738, 1743, 1752, 1761, 1763, 1769, 1776, and 1786; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1200, 1212, 1680, 1690, 1698, 1709, 1728, 1739, 1744, 1753, 1770, and 1777; or at least 65%, at least 70%, at least 75%, at least 80%, at least 85%, at least 90%, at least 95%, at least 99%, or 100% identical to any one of amino acid sequences AAS, APS, AT, ATS, DAS, DDG, DDS, DT, DTS, DVS, EAS, FTS, GAF, GAN, GAS, GAT, GIS, GKN, GNS, HTS, KVS, LAS, LGS, LVS, NAK, NTN, QMS, RMS, RTS, SAS, TTS, WAS, YAS, YAT, YGS, YRS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1097, 1104, 1111, 1121, 1128, 1135, 1142, 1149, 1156, 1163, 1170, 1175, 1182, 1189, 1195, 1201, 1207, 1213, 1222, 1229, 1236, 2009, 1248, 1255, 1262, 1267, 1272, 1279, 1286, 1293, 1300, 1307, 1320, 1327, 1333, 1340, 1346, 1353, 1360, 1367, 1374, 1383, 1389, 1395, 1402, 1409, 1415, 1422, 1429, 1435, 1442, 1449, 1456, 1462, 1469, 1476, 1482, 1489, 1496, 1502, 1509, 1513, 1520, 1532, 1539, 1545, 1557, 1562, 1569, 1575, 1582, 1588, 1595, 1602, 1609, 1616, 1623, 1630, 1637, 1644, 1651, 1658, 1663, 1673, 1681, 1691, 1699, 1710, 1729, 1735, 1745, 1751, 1754, 1778, 1787, and 1976.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1099, 1106, 1113, 1117, 1123, 1130, 1137, 1144, 1151, 1158, 1165, 1177, 1184, 1191, 1203, 1215, 1224, 1231, 1238, 1243, 1250, 1257, 1264, 1274, 1281, 1288, 1295, 1302, 1309, 1315, 1322, 1329, 1335, 1342, 1348, 1355, 1362, 1369, 1376, 1385, 1397, 1404, 1417, 1424, 1431, 1437, 1444, 1451, 1458, 1464, 1471, 1478, 1484, 1491, 1498, 1504, 1511, 1515, 1522, 1534, 1547, 1552, 1564, 1577, 1584, 1590, 1597, 1604, 1611, 1618, 1625, 1632, 1639, 1646, 1653, 1660, 1665, 1675, 1683, 1693, 1701, 1705, 1716, 1719, 1731, 1736, 1747, 1756, 1772, 1780, and 1789; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1100, 1107, 1114, 1118, 1124, 1131, 1138, 1145, 1152, 1159, 1166, 1178, 1185, 1192, 1204, 1216, 1225, 1232, 1239, 1244, 1251, 1258, 1275, 1282, 1289, 1296, 1303, 1310, 1316, 1323, 1330, 1336, 1343, 1349, 1356, 1363, 1370, 1377, 1386, 1391, 1398, 1405, 1411, 1418, 1425, 1432, 1438, 1445, 1452, 1465, 1472, 1479, 1485, 1492, 1499, 1505, 1516, 1523, 1528, 1535, 1541, 1548, 1553, 1559, 1565, 1571, 1578, 1585, 1591, 1598, 1605, 1612, 1619, 1626, 1633, 1640, 1647, 1654, 1666, 1676, 1684, 1687, 1694, 1702, 1706, 1712, 1717, 1720, 1725, 1732, 1737, 1741, 1748, 1757, 1767, 1773, 1781, and 1790; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1101, 1108, 1115, 1125, 1132, 1139, 1146, 1153, 1160, 1167, 1172, 1179, 1186, 1193, 1197, 1205, 1209, 1217, 1226, 1233, 1240, 1245, 1252, 1259, 1269, 1276, 1283, 1290, 1297, 1304, 1311, 1317, 1324, 1331, 1337, 1344, 1350, 1357, 1364, 1371, 1378, 1387, 1392, 1399, 1406, 1412, 1419, 1426, 1433, 1439, 1446, 1453, 1459, 1466, 1473, 1480, 1486, 1493, 1500, 1506, 1517, 1524, 1529, 1536, 1542, 1549, 1554, 1560, 1566, 1572, 1579, 1586, 1592, 1599, 1606, 1613, 1620, 1627, 1634, 1641, 1648, 1655, 1661, 1667, 1670, 1677, 1685, 1688, 1695, 1703, 1707, 1713, 1733, 1749, 1758, 1762, 1774, 1782, 1791, and 1975.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1098, 1105, 1112, 1122, 1129, 1136, 1143, 1150, 1157, 1164, 1171, 1176, 1183, 1190, 1196, 1202, 1208, 1214, 1219, 1223, 1230, 1237, 1242, 1249, 1256, 1263, 1268, 1273, 1280, 1287, 1294, 1301, 1308, 1314, 1321, 1328, 1334, 1341, 1347, 1354, 1361, 1368, 1375, 1380, 1384, 1390, 1396, 1403, 1410, 1416, 1423, 1430, 1436, 1443, 1450, 1457, 1463, 1470, 1477, 1483, 1490, 1497, 1503, 1510, 1514, 1521, 1527, 1533, 1540, 1546, 1551, 1558, 1563, 1570, 1576, 1583, 1589, 1596, 1603, 1610, 1617, 1624, 1631, 1638, 1645, 1652, 1659, 1664, 1669, 1674, 1682, 1692, 1700, 1711, 1721, 1724, 1730, 1740, 1746, 1755, 1764, 1766, 1771, 1779, 1784, and 1788.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 1102, 1109, 1116, 1119, 1126, 1133, 1140, 1147, 1154, 1161, 1168, 1173, 1180, 1187, 1194, 1198, 1206, 1210, 1218, 1220, 1227, 1234, 1241, 1246, 1253, 1260, 1265, 1270, 1277, 1284, 1291, 1298, 1305, 1312, 1318, 1325, 1332, 1338, 1345, 1351, 1358, 1365, 1372, 1379, 1381, 1388, 1393, 1400, 1407, 1413, 1420, 1427, 1434, 1440, 1447, 1454, 1460, 1467, 1474, 1481, 1487, 1494, 1501, 1507, 1512, 1518, 1525, 1530, 1537, 1543, 1550, 1555, 1561, 1567, 1573, 1580, 1587, 1593, 1600, 1607, 1614, 1621, 1628, 1635, 1642, 1649, 1656, 1662, 1668, 1671, 1678, 1686, 1696, 1704, 1714, 1722, 1726, 1734, 1742, 1750, 1759, 1760, 1765, 1768, 1775, 1783, 1785, and 1792.

In some aspects, the infectious disease antigen that is not a sarbecovirus antigen is:
(i) a viral antigen elected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein. F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g. p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50 (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein); or
(ii) a bacterial or parasite antigen selected from the group consisting of a Plasmodium (e.g., Plasmodium falciparum. Plasmodium vivax. Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans. Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Custoisospora (e.g., Cystoisospora belli) antigen, and another antigen (e.g., endotoxins, lymphotoxins (e.g., lymphotoxin alpha LTA), phosphatidylserine. Hsp90, CCR5, lipoteichoic acid, clumping factor A).

In some aspects, the VL1 comprises a light chain complementarity determining region 1 (LCDR1), LCDR2, and/or LCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH1 comprises a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL1 comprises a VL of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VH1 comprises a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1428, 1793, 1798, 1810, 1817, 1824, 1977, 1985, 1993 and 2001; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1978, 1986, 1994, and 2002; or at least 65%, at least 70%, at least 75%, at least 80%, at least 85%, at least 90%, at least 95%, at least 99%, or 100% identical to any one of amino acid sequences YTS, DTS, GAS, and AAS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1229, 1799, 1805, 1811, 1818, 1825, 1979, 1987, 1995, and 2003.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1577, 1795, 1801, 1813, 1820, 1827, 1981, 1989, 1997, and 2005; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1159, 1802, 1807, 1814, 1821, 1828, 1982, 1990, 1998, and 2006; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1796, 1803, 1808, 1815, 1822, 1829, 1983, 1991, 1999, and 2007.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1794, 1800, 1806, 1812, 1819, 1826, 1980, 1988, 1996, and 2004.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs 1797, 1804, 1809, 1816, 1823, 1830, 1984, 1992, 2000, and 2008.

In some aspects, the other-pathology antigen is selected from the group consisting of Amyloid beta, β-amyloid, PCSK9, IFN-α/β receptor, Rhesus factor, nerve growth factor (NGF), DPP4, fibrin II beta chain, ACVR2B, scrum amyloid A protein SOST, FGF 23, VWF, tissue factor pathway inhibitor (TFPI), 1-40 and 1-42, selectin P, glucagon receptor (GCGR), amyloid P component, GDF-8, CXCL10 IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A. Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

In some aspects, the VL1 comprises a light chain complementarity determining region 1 (LCDR1), LCDR2, and/or LCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH1 comprises a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL1 comprises a VL of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VH1 comprises a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

In some aspects, the VL1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1120, 1228, 1359, 1519, 1831, 1838, 1845, 1852, 1859, 1866, 1872, 1878, 1885, 1891, 1898, 1905, 1911, 1917, 1924, 1931, 1938, 1945, and 1963; (ii) a CDR2 having an amino acid sequence that is at least 65%, at least 70%, at least 75%, at least 80%, at least 85%, at least 90%, at least 95%, at least 99%, or 100% identical to any one of amino acid sequences AAS, AVS, DAS, DNN, EDN, EVS, GAS, GNG, KAS, KVS, LVS, NTD, STS, WAS, and YTS; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1360, 1832, 1839, 1846, 1853, 1860, 1867, 1873, 1879, 1886, 1892, 1899, 1906, 1912, 1918, 1925, 1932, 1939, 1946, 1951, 1957, 1964, and 1970.

In some aspects, the VH1 comprises (i) a complementarity determining region (CDR) 1 having an amino acid sequence that is at least 65%, at least 70%, at least 75%, at least 80%, at least 85%, at least 90%, at least 95%, at least 99%, or 100% identical to any one of SEQ ID NOs: 1309, 1362, 1437, 1552, 1618, 1639, 1834, 1841, 1848, 1855, 1862, 1881, 1894, 1901, 1920, 1927, 1934, 1941, 1953, 1959, and 1966; (ii) a CDR2 having an amino acid sequence that is at least 65%, at least 70%, at least 75%, at least 80%, at least 85%, at least 90%, at least 95%, at least 99%, or 100% identical to any one of SEQ ID NOs: 1363, 1835, 1842, 1849, 1856, 1863, 1869, 1875, 1882, 1888, 1895, 1902, 1908, 1914, 1921, 1928, 1935, 1942, 1948, 1954, 1960, 1967, and 1972; and (iii) a CDR3 having an amino acid sequence that is at least 65%, at least 70%, at least 75%, at least 80%, at least 85%, at least 90%, at least 95%, at least 99%, or 100% identical to any one of SEQ ID NOs: 1364, 1836, 1843, 1850, 1857, 1864, 1870, 1876, 1883, 1889, 1896, 1903, 1909, 1915, 1922, 1929, 1936, 1943, 1949, 1955, 1961, 1968, and 1973.

In some aspects, the VL1 comprises an amino acid sequence that is at least 65%, at least 70%, at least 75%, at least 80%, at least 85%, at least 90%, at least 95%, at least 99%, or 100% identical to any one of SEQ ID NOs: 1361, 1833, 1840, 1847, 1854, 1861, 1868, 1874, 1880, 1887, 1893, 1900, 1907, 1913, 1919, 1926, 1933, 1940, 1947, 1952, 1958, 1965, and 1971.

In some aspects, the VH1 comprises an amino acid sequence that is at least 65%, at least 70%, at least 75%, at least 80%, at least 85%, at least 90%, at least 95%, at least 99%, or 100% identical to any one of SEQ ID NOs: 1365, 1837, 1844, 1851, 1858, 1865, 1871, 1877, 1884, 1890, 1897, 1904, 1910, 1916, 1923, 1930, 1937, 1944, 1950, 1956, 1962, 1969, and 1974.

In some aspects, the antigen binding polypeptides comprised in the antibodies or antigen binding fragments thereof (e.g., monospecific or multispecific antibodies or antigen binding fragments thereof) provided herein further comprise an Fc region. In some aspects, the antigen binding polypeptides comprised in the antibodies or antigen binding fragments thereof (e.g., monospecific or multispecific antibodies or antigen binding fragments thereof) provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 80%, at least 65%, at least 70%, at least 75%, at least 80%, at least 85%, at least 90%, at least 95%, at least 99%, or 100% identical to any one of SEQ ID NOs: 376-380.

In some aspects, the Fc region comprises one or more amino acid substitutions. In some aspects, the one or more amino acid substitutions comprise one or more half-life extension amino acid substitutions. In some aspects, the one or more half-life extension amino acid substitutions comprise one or more of amino acid substitutions M428L, N434A, N434S, L234F, L235E, P331S, M252Y, S254T, or T256E, or equivalent thereof, based on the EU numbering scheme. In some aspects, the one or more half-life extension amino acid substitutions comprise a LA (M428L and N434A, or equivalent thereof, based on the EU numbering scheme) amino acid substitution. In some aspects, the one or more half-life extension amino acid substitutions comprise a LS (M428L and N434S, or equivalent thereof, based on the EU numbering scheme) amino acid substitution. In some aspects, the one or more half-life extension amino acid substitutions comprise L234F and L235E, or equivalent thereof (based on the EU numbering scheme) amino acid substitutions. In some aspects, the one or more half-life extension amino acid substitutions comprise a TM (L234F, L235E, and P331S, or equivalent thereof, based on the EU numbering scheme) amino acid substitution. In some aspects, the one or more half-life extension amino acid substitutions comprise a YTE (M252Y, S254T, and T256E, or equivalent thereof, based on the EU numbering scheme) amino acid substitution.

In some aspects, the Fc region comprises at least one knob-into-hole modification. In some aspects, the antigen binding polypeptides are IgG1 or IgG4 antibodies or antigen binding fragments thereof. In some aspects, the knob-into-hole modification comprises one or more of knob substitutions of S354C and T366W, or equivalent thereof, and/or one or more of hole substitutions of Y349C, T366S, L368A, Y407V, or equivalent thereof; or a combination thereof, based on the EU numbering scheme. In some aspects, the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, or equivalent thereof, and T256E, based on the EU numbering scheme) amino acid substitution, or any combination thereof.

In some aspects, the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In some aspects, the antigen binding polypeptides comprised in the antibodies or antigen binding fragments thereof (e.g., monospecific or multispecific antibodies or antigen binding fragments thereof) disclosed herein comprise one or more Fc effector function knockout mutation. In some aspects, the antigen binding polypeptides comprised in the antibodies or antigen binding fragments thereof (e.g., monospecific or multispecific antibodies or antigen binding fragments thereof) disclosed herein are IgG1 or IgG4 antibodies or antigen binding fragments thereof. In some aspects, the Fc effector function knockout mutation is L234A, L235A, P239A, or equivalent thereof, or a combination thereof, based on the EU numbering scheme.

In some aspects, the antigen binding polypeptides comprised in the antibodies or antigen binding fragments thereof (e.g., monospecific or multispecific antibodies or antigen binding fragments thereof) disclosed herein comprise a mutation at a glycosylation site. In some aspects, the mutation at a glycosylation site is a mutation at an N-glycosylation site. In some aspects, the antigen binding polypeptides comprised in the antibodies or antigen binding fragments thereof (e.g., monospecific or multispecific antibodies or antigen binding fragments thereof) disclosed herein comprise a VH comprising a mutation at a glycosylation site. In some aspects, the mutation at a glycosylation site is a mutation at an N-glycosylation site. In some aspects, the mutation at an N-glycosylation site is at amino acid N62 of the VH region or equivalent thereof. In some aspects, the mutation at an N-glycosylation site at amino acid N62 is N62Q or N62S, or equivalent thereof.

In some aspects, the antigen binding polypeptides comprised in the antibodies or antigen binding fragments thereof (e.g., monospecific or multispecific antibodies or antigen binding fragments thereof) disclosed herein, comprise one or more optional linkers. The one or more optional linkers are indicated herein as Ln, wherein n is an integer, and the integer next to each L indicates each L comprised in the antigen binding polypeptides comprised in the antigen binding polypeptide complexes disclosed herein. For example, if an antigen binding polypeptide comprised in the antigen binding polypeptide complexes disclosed herein comprises two linkers, the first linker from N-terminus to C-terminus is herein indicated as L1, and the second linker from N-terminus to C-terminus is herein indicated as L2. If an antigen binding polypeptide complex disclosed herein comprises a first antigen binding polypeptide comprising two linkers, indicated herein as L1 and L2, as explained above, the linkers comprised in the second antigen binding polypeptide are indicated with the following integer, in this example, the first linker from N-terminus to C-terminus of the second antigen binding polypeptide is herein indicated as L3, the second linker from N-terminus to C-terminus of the second antigen binding polypeptide is herein indicated as L3. In some aspects, any one of the optional linkers each independently have a length of from 0 amino acids to about 50 amino acids. In some aspects, any one of the optional linkers each independently have a length of from 5 amino acids to about 40 amino acids.

In some aspects, any one of the optional linkers are non-immunogenic. In some aspects, any one of the optional linkers do not contain a consensus T cell epitope.

In some aspects, any of Ln comprises an amino acid sequence of G or A, or an amino acid sequence at least 65%, at least 70%, at least 75%, at least 80%, at least 85%, at least 90%, at least 95%, at least 99%, or 100% identical to amino acid sequence GSS or ASG, or an amino acid sequence at least 65%, at least 70%, at least 75%, at least 80%, at least 85%, at least 90%, at least 95%, at least 99%, or 100% identical to any one of SEQ ID NOs: 333-373 or 967-971. In some aspects. Ln comprises an amino acid sequence at least 65%, at least 70%, at least 75%, at least 80%, at least 85%, at least 90%, at least 95%, at least 99%, or 100% identical to any one of SEQ ID NOs: 333-345 or 967-971.

In some aspects, the antigen binding polypeptide is selected from any one of the more specific aspects provided herein for an antigen binding polypeptide in an antigen binding polypeptide complex having no more than three polypeptide chains.

In some aspects, the antigen binding polypeptide further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptide is selected from any one of the more specific aspects provided herein for an antigen binding polypeptide in an antigen binding polypeptide complex having no more than three polypeptide chains, and further comprises one or more immunoglobulin heavy chain constant region 1 (CH1) and/or one or more immunoglobulin light chain constant region (CL) between any one of the variable regions and/or optional amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between. L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

In some aspects, the antigen binding polypeptides comprised in the antibodies or antigen binding fragments thereof (e.g., monospecific or multispecific antibodies or antigen binding fragments thereof) disclosed herein, comprise one or more CL, as indicated in the formulas representing the antigen binding polypeptide complexes disclosed herein. In some aspects, the CL comprises an amino acid sequence at least 65%, at least 70%, at least 75%, at least 80%, at least 85%, at least 90%, at least 95%, at least 99%, or 100% identical to any one of SEQ ID NOs: 374, 637, or 1092-1095.

In some aspects, the antigen binding polypeptides comprised in the antibodies or antigen binding fragments thereof (e.g., monospecific or multispecific antibodies or antigen binding fragments thereof) disclosed herein, comprise one or more CH1, as indicated in the formulas representing the antigen binding polypeptide complexes disclosed herein. In some aspects, the CH1 comprises an amino acid sequence at least 65%, at least 70%, at least 75%, at least 80%, at least 85%, at least 90%, at least 95%, at least 99%, or 100% identical to any one of SEQ ID NOs: 375, 634, 1070, 1071, or 1086-1091.

In some aspects, the antigen binding polypeptides comprised in the antibodies or antigen binding fragments thereof (e.g., monospecific or multispecific antibodies or antigen binding fragments thereof) provided herein further comprise an Fc region. In some aspects, the antigen binding polypeptides comprised in the antibodies or antigen binding fragments thereof (e.g., monospecific or multispecific antibodies or antigen binding fragments thereof) provided herein further comprise an Fc region at the carboxy-terminus. In some aspects, the Fc region comprises an immunoglobulin heavy chain constant region 2 (CH2) and/or an immunoglobulin heavy chain constant region 3 (CH3). In some aspects, the Fc region further comprises an immunoglobulin hinge (i.e., a hinge region). In some aspects, the hinge region is interposed between a CH1 and a CH2 (i.e., a hinge region is localized between the carboxy terminal residue of a CH1 and the N-terminal residue of a CH2). In some aspects, the hinge region comprises an amino acid sequence at least 65%, at least 70%, at least 75%, at least 80%, at least 85%, at least 90%, at least 95%, at least 99%, or 100% identical to any one of SEQ ID NOs: 635, 636, 1072, 1073, or 1074. In some aspects, the Fc region comprises a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085. In some aspects, the Fc region comprises an amino acid sequence that is at least 65%, at least 70%, at least 75%, at least 80%, at least 85%, at least 90%, at least 95%, at least 99%, or 100% identical to any one of SEQ ID NOs: 376-380.

In some aspects, any of the antigen binding polypeptides, antigen binding polypeptide complexes, or antibodies or antigen binding fragments thereof disclosed herein comprises:
a LCDR1 at least 65%, at least 70%, at least 75%, at least 80%, at least 85%, at least 90%, at least 95%, at least 99%, or 100% identical to SEQ ID NO: 1096, a LCDR2 at least 65%, at least 70%, at least 75%, at least 80%, at least 85%, at least 90%, at least 95%, at least 99%, or 100% identical to amino acid sequence GKN, a LCDR3 at least 65%, at least 70%, at least 75%, at least 80%, at least 85%, at least 90%, at least 95%, at least 99%, or 100% identical to SEQ ID NO: 1097, a HCDR1 at least 65%, at least 70%, at least 75%, at least 80%, at least 85%, at least 90%, at least 95%, at least 99%, or 100% identical to SEQ ID NO: 1099, a HCDR2 at least 65%, at least 70%, at least 75%, at least 80%, at least 85%, at least 90%, at least 95%, at least 99%, or 100% identical to SEQ ID NO: 1100, and a HCDR3 at least 65%, at least 70%, at least 75%, at least 80%, at least 85%, at least 90%, at least 95%, at least 99%, or 100% identical to SEQ ID NO: 1101;
a LCDR1 at least 65%, at least 70%, at least 75%, at least 80%, at least 85%, at least 90%, at least 95%, at least 99%, or 100% identical to SEQ ID NO: 1103, a LCDR2 at least 65%, at least 70%, at least 75%, at least 80%, at least 85%, at least 90%, at least 95%, at least 99%, or 100% identical to amino acid sequence GAT, a LCDR3 at least 65%, at least 70%, at least 75%, at least 80%, at least 85%, at least 90%, at least 95%, at least 99%, or 100% identical to SEQ ID NO: 1104, a HCDR1 at least 65%, at least 70%, at least 75%, at least 80%, at least 85%, at least 90%, at least 95%, at least 99%, or 100% identical to SEQ ID NO: 1106, a HCDR2 at least 65%, at least 70%, at least 75%, at least 80%, at least 85%, at least 90%, at least 95%, at least 99%, or 100% identical to SEQ ID NO: 1107, and a HCDR3 at least 65%, at least 70%, at least 75%, at least 80%, at least 85%, at least 90%, at least 95%, at least 99%, or 100% identical to SEQ ID NO: 1108;
a LCDR1 at least 65%, at least 70%, at least 75%, at least 80%, at least 85%, at least 90%, at least 95%, at least 99%, or 100% identical to SEQ ID NO: 1110, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1111, a HCDR1 at least 65%, at least 70%, at least 75%, at least 80%, at least 85%, at least 90%, at least 95%, at least 99%, or 100% identical to SEQ ID NO: 1113, a HCDR2 at least 65%, at least 70%, at least 75%, at least 80%, at least 85%, at least 90%, at least 95%, at least 99%, or 100% identical to SEQ ID NO: 1114, and a HCDR3 at least 65%, at least 70%, at least 75%, at least 80%, at least 85%, at least 90%, at least 95%, at least 99%, or 100% identical to SEQ ID NO: 1115;
a LCDR1 at least 65%, at least 70%, at least 75%, at least 80%, at least 85%, at least 90%, at least 95%, at least 99%, or 100% identical to SEQ ID NO: 1103, a LCDR2 at least 65%, at least 70%, at least 75%, at least 80%, at least 85%, at least 90%, at least 95%, at least 99%, or 100% identical to amino acid sequence GAT, a LCDR3 at least 65%, at least 70%, at least 75%, at least 80%, at least 85%, at least 90%, at least 95%, at least 99%, or 100% identical to SEQ ID NO: 1104, a HCDR1 at least 65%, at least 70%, at least 75%, at least 80%, at least 85%, at least 90%, at least 95%, at least 99%, or 100% identical to SEQ ID NO: 1117, a HCDR2 at least 65%, at least 70%, at least 75%, at least 80%, at least 85%, at least 90%, at least 95%, at least 99%, or 100% identical to SEQ ID NO: 1118, and a HCDR3 at least 65%, at least 70%, at least 75%, at least 80%, at least 85%, at least 90%, at least 95%, at least 99%, or 100% identical to SEQ ID NO: 1108;
a LCDR1 at least 65%, at least 70%, at least 75%, at least 80%, at least 85%, at least 90%, at least 95%, at least 99%, or 100% identical to SEQ ID NO: 1120, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1121, a HCDR1 at least 65%, at least 70%, at least 75%, at least 80%, at least 85%, at least 90%, at least 95%, at least 99%, or 100% identical to SEQ ID NO: 1123, a HCDR2 at least 65%, at least 70%, at least 75%, at least 80%, at least 85%, at least 90%, at least 95%, at least 99%, or 100% identical to SEQ ID NO: 1124, and a HCDR3 at least 65%, at least 70%, at least 75%, at least 80%, at least 85%, at least 90%, at least 95%, at least 99%, or 100% identical to SEQ ID NO: 1125;
a LCDR1 at least 65%, at least 70%, at least 75%, at least 80%, at least 85%, at least 90%, at least 95%, at least 99%, or 100% identical to SEQ ID NO: 1127, a LCDR2 at least 65%, at least 70%, at least 75%, at least 80%, at least 85%, at least 90%, at least 95%, at least 99%, or 100% identical to amino acid sequence KVS, a LCDR3 at least 65%, at least 70%, at least 75%, at least 80%, at least 85%, at least 90%, at least 95%, at least 99%, or 100% identical to SEQ ID NO: 1128, a HCDR1 at least 65%, at least 70%, at least 75%, at least 80%, at least 85%, at least 90%, at least 95%, at least 99%, or 100% identical to SEQ ID NO: 1130, a HCDR2 at least 65%, at least 70%, at least 75%, at least 80%, at least 85%, at least 90%, at least 95%, at least 99%, or 100% identical to SEQ ID NO: 1131, and a HCDR3 at least 65%, at least 70%, at least 75%, at least 80%, at least 85%, at least 90%, at least 95%, at least 99%, or 100% identical to SEQ ID NO: 1132;
a LCDR1 at least 65%, at least 70%, at least 75%, at least 80%, at least 85%, at least 90%, at least 95%, at least 99%, or 100% identical to SEQ ID NO: 1134, a LCDR2 at least 65%, at least 70%, at least 75%, at least 80%, at least 85%, at least 90%, at least 95%, at least 99%, or 100% identical to amino acid sequence LAS, a LCDR3 at least 65%, at least 70%, at least 75%, at least 80%, at least 85%, at least 90%, at least 95%, at least 99%, or 100% identical to SEQ ID NO: 1135, a HCDR1 at least 65%, at least 70%, at least 75%, at least 80%, at least 85%, at least 90%, at least 95%, at least 99%, or 100% identical to SEQ ID NO: 1137, a HCDR2 at least 65%, at least 70%, at least 75%, at least 80%, at least 85%, at least 90%, at least 95%, at least 99%, or 100% identical to SEQ ID NO: 1138, and a HCDR3 at least 65%, at least 70%, at least 75%, at least 80%, at least 85%, at least 90%, at least 95%, at least 99%, or 100% identical to SEQ ID NO: 1139;
a LCDR1 at least 65%, at least 70%, at least 75%, at least 80%, at least 85%, at least 90%, at least 95%, at least 99%, or 100% identical to SEQ ID NO: 1141, a LCDR2 at least 65%, at least 70%, at least 75%, at least 80%, at least 85%, at least 90%, at least 95%, at least 99%, or 100% identical to amino acid sequence HTS, a LCDR3 at least 65%, at least 70%, at least 75%, at least 80%, at least 85%, at least 90%, at least 95%, at least 99%, or 100% identical to SEQ ID NO: 1142, a HCDR1 at least 65%, at least 70%, at least 75%, at least 80%, at least 85%, at least 90%, at least 95%, at least 99%, or 100% identical to SEQ ID NO: 1144, a HCDR2 at least 65%, at least 70%, at least 75%, at least 80%, at least 85%, at least 90%, at least 95%, at least 99%, or 100% identical to SEQ ID NO: 1145, and a HCDR3 at least 65%, at least 70%, at least 75%, at least 80%, at least 85%, at least 90%, at least 95%, at least 99%, or 100% identical to SEQ ID NO: 1146;
a LCDR1 at least 65%, at least 70%, at least 75%, at least 80%, at least 85%, at least 90%, at least 95%, at least 99%, or 100% identical to SEQ ID NO: 1148, a LCDR2 at least 65%, at least 70%, at least 75%, at least 80%, at least 85%, at least 90%, at least 95%, at least 99%, or 100% identical to amino acid sequence YAS, a LCDR3 at least 65%, at least 70%, at least 75%, at least 80%, at least 85%, at least 90%, at least 95%, at least 99%, or 100% identical to SEQ ID NO: 1149, a HCDR1 at least 65%, at least 70%, at least 75%, at least 80%, at least 85%, at least 90%, at least 95%, at least 99%, or 100% identical to SEQ ID NO: 1151, a HCDR2 at least 65%, at least 70%, at least 75%, at least 80%, at least 85%, at least 90%, at least 95%, at least 99%, or 100% identical to SEQ ID NO: 1152, and a HCDR3 at least 65%, at least 70%, at least 75%, at least 80%, at least 85%, at least 90%, at least 95%, at least 99%, or 100% identical to SEQ ID NO: 1153;
a LCDR1 at least 65%, at least 70%, at least 75%, at least 80%, at least 85%, at least 90%, at least 95%, at least 99%, or 100% identical to SEQ ID NO: 1155, a LCDR2 at least 65%, at least 70%, at least 75%, at least 80%, at least 85%, at least 90%, at least 95%, at least 99%, or 100% identical to amino acid sequence EAS, a LCDR3 at least 65%, at least 70%, at least 75%, at least 80%, at least 85%, at least 90%, at least 95%, at least 99%, or 100% identical to SEQ ID NO: 1156, a HCDR1 at least 65%, at least 70%, at least 75%, at least 80%, at least 85%, at least 90%, at least 95%, at least 99%, or 100% identical to SEQ ID NO: 1158, a HCDR2 at least 65%, at least 70%, at least 75%, at least 80%, at least 85%, at least 90%, at least 95%, at least 99%, or 100% identical to SEQ ID NO: 1159, and a HCDR3 at least 65%, at least 70%, at least 75%, at least 80%, at least 85%, at least 90%, at least 95%, at least 99%, or 100% identical to SEQ ID NO: 1160;
a LCDR1 at least 65%, at least 70%, at least 75%, at least 80%, at least 85%, at least 90%, at least 95%, at least 99%, or 100% identical to SEQ ID NO: 1162, a LCDR2 at least 65%, at least 70%, at least 75%, at least 80%, at least 85%, at least 90%, at least 95%, at least 99%, or 100% identical to amino acid sequence DTS, a LCDR3 at least 65%, at least 70%, at least 75%, at least 80%, at least 85%, at least 90%, at least 95%, at least 99%, or 100% identical to SEQ ID NO: 1163, a HCDR1 at least 65%, at least 70%, at least 75%, at least 80%, at least 85%, at least 90%, at least 95%, at least 99%, or 100% identical to SEQ ID NO: 1165, a HCDR2 at least 65%, at least 70%, at least 75%, at least 80%, at least 85%, at least 90%, at least 95%, at least 99%, or 100% identical to SEQ ID NO: 1166, and a HCDR3 at least 65%, at least 70%, at least 75%, at least 80%, at least 85%, at least 90%, at least 95%, at least 99%, or 100% identical to SEQ ID NO: 1167;
a LCDR1 at least 65%, at least 70%, at least 75%, at least 80%, at least 85%, at least 90%, at least 95%, at least 99%, or 100% identical to SEQ ID NO: 1169, a LCDR2 at least 65%, at least 70%, at least 75%, at least 80%, at least 85%, at least 90%, at least 95%, at least 99%, or 100% identical to amino acid sequence RMS, a LCDR3 at least 65%, at least 70%, at least 75%, at least 80%, at least 85%, at least 90%, at least 95%, at least 99%, or 100% identical to SEQ ID NO: 1170, a HCDR1 at least 65%, at least 70%, at least 75%, at least 80%, at least 85%, at least 90%, at least 95%, at least 99%, or 100% identical to SEQ ID NO: 1144, a HCDR2 at least 65%, at least 70%, at least 75%, at least 80%, at least 85%, at least 90%, at least 95%, at least 99%, or 100% identical to SEQ ID NO: 1145, and a HCDR3 at least 65%, at least 70%, at least 75%, at least 80%, at least 85%, at least 90%, at least 95%, at least 99%, or 100% identical to SEQ ID NO: 1172;
a LCDR1 at least 65%, at least 70%, at least 75%, at least 80%, at least 85%, at least 90%, at least 95%, at least 99%, or 100% identical to SEQ ID NO: 1174, a LCDR2 at least 65%, at least 70%, at least 75%, at least 80%, at least 85%, at least 90%, at least 95%, at least 99%, or 100% identical to amino acid sequence QMS, a LCDR3 at least 65%, at least 70%, at least 75%, at least 80%, at least 85%, at least 90%, at least 95%, at least 99%, or 100% identical to SEQ ID NO: 1175, a HCDR1 at least 65%, at least 70%, at least 75%, at least 80%, at least 85%, at least 90%, at least 95%, at least 99%, or 100% identical to SEQ ID NO: 1177, a HCDR2 at least 65%, at least 70%, at least 75%, at least 80%, at least 85%, at least 90%, at least 95%, at least 99%, or 100% identical to SEQ ID NO: 1178, and a HCDR3 at least 65%, at least 70%, at least 75%, at least 80%, at least 85%, at least 90%, at least 95%, at least 99%, or 100% identical to SEQ ID NO: 1179;
a LCDR1 at least 65%, at least 70%, at least 75%, at least 80%, at least 85%, at least 90%, at least 95%, at least 99%, or 100% identical to SEQ ID NO: 1181, a LCDR2 at least 65%, at least 70%, at least 75%, at least 80%, at least 85%, at least 90%, at least 95%, at least 99%, or 100% identical to amino acid sequence APS, a LCDR3 at least 65%, at least 70%, at least 75%, at least 80%, at least 85%, at least 90%, at least 95%, at least 99%, or 100% identical to SEQ ID NO: 1182, a HCDR1 at least 65%, at least 70%, at least 75%, at least 80%, at least 85%, at least 90%, at least 95%, at least 99%, or 100% identical to SEQ ID NO: 1184, a HCDR2 at least 65%, at least 70%, at least 75%, at least 80%, at least 85%, at least 90%, at least 95%, at least 99%, or 100% identical to SEQ ID NO: 1185, and a HCDR3 at least 65%, at least 70%, at least 75%, at least 80%, at least 85%, at least 90%, at least 95%, at least 99%, or 100% identical to SEQ ID NO: 1186;
a LCDR1 at least 65%, at least 70%, at least 75%, at least 80%, at least 85%, at least 90%, at least 95%, at least 99%, or 100% identical to SEQ ID NO: 1188, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1189, a HCDR1 at least 65%, at least 70%, at least 75%, at least 80%, at least 85%, at least 90%, at least 95%, at least 99%, or 100% identical to SEQ ID NO: 1191, a HCDR2 at least 65%, at least 70%, at least 75%, at least 80%, at least 85%, at least 90%, at least 95%, at least 99%, or 100% identical to SEQ ID NO: 1192, and a HCDR3 at least 65%, at least 70%, at least 75%, at least 80%, at least 85%, at least 90%, at least 95%, at least 99%, or 100% identical to SEQ ID NO: 1193;
a LCDR1 at least 65%, at least 70%, at least 75%, at least 80%, at least 85%, at least 90%, at least 95%, at least 99%, or 100% identical to SEQ ID NO: 1181, a LCDR2 at least 65%, at least 70%, at least 75%, at least 80%, at least 85%, at least 90%, at least 95%, at least 99%, or 100% identical to amino acid sequence ATS, a LCDR3 at least 65%, at least 70%, at least 75%, at least 80%, at least 85%, at least 90%, at least 95%, at least 99%, or 100% identical to SEQ ID NO: 1195, a HCDR1 at least 65%, at least 70%, at least 75%, at least 80%, at least 85%, at least 90%, at least 95%, at least 99%, or 100% identical to SEQ ID NO: 1184, a HCDR2 at least 65%, at least 70%, at least 75%, at least 80%, at least 85%, at least 90%, at least 95%, at least 99%, or 100% identical to SEQ ID NO: 1185, and a HCDR3 at least 65%, at least 70%, at least 75%, at least 80%, at least 85%, at least 90%, at least 95%, at least 99%, or 100% identical to SEQ ID NO: 1197;
a LCDR1 at least 65%, at least 70%, at least 75%, at least 80%, at least 85%, at least 90%, at least 95%, at least 99%, or 100% identical to SEQ ID NO: 1199, a LCDR2 at least 65%, at least 70%, at least 75%, at least 80%, at least 85%, at least 90%, at least 95%, at least 99%, or 100% identical to SEQ ID NO: 1200, a LCDR3 at least 65%, at least 70%, at least 75%, at least 80%, at least 85%, at least 90%, at least 95%, at least 99%, or 100% identical to SEQ ID NO: 1201, a HCDR1 at least 65%, at least 70%, at least 75%, at least 80%, at least 85%, at least 90%, at least 95%, at least 99%, or 100% identical to SEQ ID NO: 1203, a HCDR2 at least 65%, at least 70%, at least 75%, at least 80%, at least 85%, at least 90%, at least 95%, at least 99%, or 100% identical to SEQ ID NO: 1204, and a HCDR3 at least 65%, at least 70%, at least 75%, at least 80%, at least 85%, at least 90%, at least 95%, at least 99%, or 100% identical to SEQ ID NO: 1205;
a LCDR1 at least 65%, at least 70%, at least 75%, at least 80%, at least 85%, at least 90%, at least 95%, at least 99%, or 100% identical to SEQ ID NO: 1181, a LCDR2 at least 65%, at least 70%, at least 75%, at least 80%, at least 85%, at least 90%, at least 95%, at least 99%, or 100% identical to amino acid sequence ATS, a LCDR3 at least 65%, at least 70%, at least 75%, at least 80%, at least 85%, at least 90%, at least 95%, at least 99%, or 100% identical to SEQ ID NO: 1207, a HCDR1 at least 65%, at least 70%, at least 75%, at least 80%, at least 85%, at least 90%, at least 95%, at least 99%, or 100% identical to SEQ ID NO: 1184, a HCDR2 at least 65%, at least 70%, at least 75%, at least 80%, at least 85%, at least 90%, at least 95%, at least 99%, or 100% identical to SEQ ID NO: 1185, and a HCDR3 at least 65%, at least 70%, at least 75%, at least 80%, at least 85%, at least 90%, at least 95%, at least 99%, or 100% identical to SEQ ID NO: 1209;
a LCDR1 at least 65%, at least 70%, at least 75%, at least 80%, at least 85%, at least 90%, at least 95%, at least 99%, or 100% identical to SEQ ID NO: 1211, a LCDR2 at least 65%, at least 70%, at least 75%, at least 80%, at least 85%, at least 90%, at least 95%, at least 99%, or 100% identical to SEQ ID NO: 1212, a LCDR3 at least 65%, at least 70%, at least 75%, at least 80%, at least 85%, at least 90%, at least 95%, at least 99%, or 100% identical to SEQ ID NO: 1213, a HCDR1 at least 65%, at least 70%, at least 75%, at least 80%, at least 85%, at least 90%, at least 95%, at least 99%, or 100% identical to SEQ ID NO: 1215, a HCDR2 at least 65%, at least 70%, at least 75%, at least 80%, at least 85%, at least 90%, at least 95%, at least 99%, or 100% identical to SEQ ID NO: 1216, and a HCDR3 at least 65%, at least 70%, at least 75%, at least 80%, at least 85%, at least 90%, at least 95%, at least 99%, or 100% identical to SEQ ID NO: 1217;
a LCDR1 at least 65%, at least 70%, at least 75%, at least 80%, at least 85%, at least 90%, at least 95%, at least 99%, or 100% identical to SEQ ID NO: 1181, a LCDR2 at least 65%, at least 70%, at least 75%, at least 80%, at least 85%, at least 90%, at least 95%, at least 99%, or 100% identical to amino acid sequence ATS, a LCDR3 at least 65%, at least 70%, at least 75%, at least 80%, at least 85%, at least 90%, at least 95%, at least 99%, or 100% identical to SEQ ID NO: 1195, a HCDR1 at least 65%, at least 70%, at least 75%, at least 80%, at least 85%, at least 90%, at least 95%, at least 99%, or 100% identical to SEQ ID NO: 1184, a HCDR2 at least 65%, at least 70%, at least 75%, at least 80%, at least 85%, at least 90%, at least 95%, at least 99%, or 100% identical to SEQ ID NO: 1185, and a HCDR3 at least 65%, at least 70%, at least 75%, at least 80%, at least 85%, at least 90%, at least 95%, at least 99%, or 100% identical to SEQ ID NO: 1197;
a LCDR1 at least 65%, at least 70%, at least 75%, at least 80%, at least 85%, at least 90%, at least 95%, at least 99%, or 100% identical to SEQ ID NO: 1221, a LCDR2 at least 65%, at least 70%, at least 75%, at least 80%, at least 85%, at least 90%, at least 95%, at least 99%, or 100% identical to amino acid sequence GIS, a LCDR3 at least 65%, at least 70%, at least 75%, at least 80%, at least 85%, at least 90%, at least 95%, at least 99%, or 100% identical to SEQ ID NO: 1222, a HCDR1 at least 65%, at least 70%, at least 75%, at least 80%, at least 85%, at least 90%, at least 95%, at least 99%, or 100% identical to SEQ ID NO: 1224, a HCDR2 at least 65%, at least 70%, at least 75%, at least 80%, at least 85%, at least 90%, at least 95%, at least 99%, or 100% identical to SEQ ID NO: 1225, and a HCDR3 at least 65%, at least 70%, at least 75%, at least 80%, at least 85%, at least 90%, at least 95%, at least 99%, or 100% identical to SEQ ID NO: 1226;
a LCDR1 at least 65%, at least 70%, at least 75%, at least 80%, at least 85%, at least 90%, at least 95%, at least 99%, or 100% identical to SEQ ID NO: 1228, a LCDR2 at least 65%, at least 70%, at least 75%, at least 80%, at least 85%, at least 90%, at least 95%, at least 99%, or 100% identical to amino acid sequence YTS, a LCDR3 at least 65%, at least 70%, at least 75%, at least 80%, at least 85%, at least 90%, at least 95%, at least 99%, or 100% identical to SEQ ID NO: 1229, a HCDR1 at least 65%, at least 70%, at least 75%, at least 80%, at least 85%, at least 90%, at least 95%, at least 99%, or 100% identical to SEQ ID NO: 1231, a HCDR2 at least 65%, at least 70%, at least 75%, at least 80%, at least 85%, at least 90%, at least 95%, at least 99%, or 100% identical to SEQ ID NO: 1232, and a HCDR3 at least 65%, at least 70%, at least 75%, at least 80%, at least 85%, at least 90%, at least 95%, at least 99%, or 100% identical to SEQ ID NO: 1233;
a LCDR1 at least 65%, at least 70%, at least 75%, at least 80%, at least 85%, at least 90%, at least 95%, at least 99%, or 100% identical to SEQ ID NO: 1235, a LCDR2 at least 65%, at least 70%, at least 75%, at least 80%, at least 85%, at least 90%, at least 95%, at least 99%, or 100% identical to amino acid sequence TTS, a LCDR3 at least 65%, at least 70%, at least 75%, at least 80%, at least 85%, at least 90%, at least 95%, at least 99%, or 100% identical to SEQ ID NO: 1236, a HCDR1 at least 65%, at least 70%, at least 75%, at least 80%, at least 85%, at least 90%, at least 95%, at least 99%, or 100% identical to SEQ ID NO: 1238, a HCDR2 at least 65%, at least 70%, at least 75%, at least 80%, at least 85%, at least 90%, at least 95%, at least 99%, or 100% identical to SEQ ID NO: 1239, and a HCDR3 at least 65%, at least 70%, at least 75%, at least 80%, at least 85%, at least 90%, at least 95%, at least 99%, or 100% identical to SEQ ID NO: 1240;
a LCDR1 at least 65%, at least 70%, at least 75%, at least 80%, at least 85%, at least 90%, at least 95%, at least 99%, or 100% identical to SEQ ID NO: 1181, a LCDR2 at least 65%, at least 70%, at least 75%, at least 80%, at least 85%, at least 90%, at least 95%, at least 99%, or 100% identical to amino acid sequence DTS, a LCDR3 at least 65%, at least 70%, at least 75%, at least 80%, at least 85%, at least 90%, at least 95%, at least 99%, or 100% identical to SEQ ID NO: 2009, a HCDR1 at least 65%, at least 70%, at least 75%, at least 80%, at least 85%, at least 90%, at least 95%, at least 99%, or 100% identical to SEQ ID NO: 1243, a HCDR2 at least 65%, at least 70%, at least 75%, at least 80%, at least 85%, at least 90%, at least 95%, at least 99%, or 100% identical to SEQ ID NO: 1244, and a HCDR3 at least 65%, at least 70%, at least 75%, at least 80%, at least 85%, at least 90%, at least 95%, at least 99%, or 100% identical to SEQ ID NO: 1245;
a LCDR1 at least 65%, at least 70%, at least 75%, at least 80%, at least 85%, at least 90%, at least 95%, at least 99%, or 100% identical to SEQ ID NO: 1247, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1248, a HCDR1 at least 65%, at least 70%, at least 75%, at least 80%, at least 85%, at least 90%, at least 95%, at least 99%, or 100% identical to SEQ ID NO: 1250, a HCDR2 at least 65%, at least 70%, at least 75%, at least 80%, at least 85%, at least 90%, at least 95%, at least 99%, or 100% identical to SEQ ID NO: 1251, and a HCDR3 at least 65%, at least 70%, at least 75%, at least 80%, at least 85%, at least 90%, at least 95%, at least 99%, or 100% identical to SEQ ID NO: 1252;
a LCDR1 at least 65%, at least 70%, at least 75%, at least 80%, at least 85%, at least 90%, at least 95%, at least 99%, or 100% identical to SEQ ID NO: 1254, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1255, a HCDR1 at least 65%, at least 70%, at least 75%, at least 80%, at least 85%, at least 90%, at least 95%, at least 99%, or 100% identical to SEQ ID NO: 1257, a HCDR2 at least 65%, at least 70%, at least 75%, at least 80%, at least 85%, at least 90%, at least 95%, at least 99%, or 100% identical to SEQ ID NO: 1258, and a HCDR3 at least 65%, at least 70%, at least 75%, at least 80%, at least 85%, at least 90%, at least 95%, at least 99%, or 100% identical to SEQ ID NO: 1259;
a LCDR1 at least 65%, at least 70%, at least 75%, at least 80%, at least 85%, at least 90%, at least 95%, at least 99%, or 100% identical to SEQ ID NO: 1261, a LCDR2 at least 65%, at least 70%, at least 75%, at least 80%, at least 85%, at least 90%, at least 95%, at least 99%, or 100% identical to amino acid sequence SAS, a LCDR3 at least 65%, at least 70%, at least 75%, at least 80%, at least 85%, at least 90%, at least 95%, at least 99%, or 100% identical to SEQ ID NO: 1262, a HCDR1 at least 65%, at least 70%, at least 75%, at least 80%, at least 85%, at least 90%, at least 95%, at least 99%, or 100% identical to SEQ ID NO: 1264, a HCDR2 at least 65%, at least 70%, at least 75%, at least 80%, at least 85%, at least 90%, at least 95%, at least 99%, or 100% identical to SEQ ID NO: 1159, and a HCDR3 at least 65%, at least 70%, at least 75%, at least 80%, at least 85%, at least 90%, at least 95%, at least 99%, or 100% identical to SEQ ID NO: 1975;
a LCDR1 at least 65%, at least 70%, at least 75%, at least 80%, at least 85%, at least 90%, at least 95%, at least 99%, or 100% identical to SEQ ID NO: 1266, a LCDR2 at least 65%, at least 70%, at least 75%, at least 80%, at least 85%, at least 90%, at least 95%, at least 99%, or 100% identical to amino acid sequence WAS, a LCDR3 at least 65%, at least 70%, at least 75%, at least 80%, at least 85%, at least 90%, at least 95%, at least 99%, or 100% identical to SEQ ID NO: 1267, a HCDR1 at least 65%, at least 70%, at least 75%, at least 80%, at least 85%, at least 90%, at least 95%, at least 99%, or 100% identical to SEQ ID NO: 1144, a HCDR2 at least 65%, at least 70%, at least 75%, at least 80%, at least 85%, at least 90%, at least 95%, at least 99%, or 100% identical to SEQ ID NO: 1145, and a HCDR3 at least 65%, at least 70%, at least 75%, at least 80%, at least 85%, at least 90%, at least 95%, at least 99%, or 100% identical to SEQ ID NO: 1269;
a LCDR1 at least 65%, at least 70%, at least 75%, at least 80%, at least 85%, at least 90%, at least 95%, at least 99%, or 100% identical to SEQ ID NO: 1271, a LCDR2 at least 65%, at least 70%, at least 75%, at least 80%, at least 85%, at least 90%, at least 95%, at least 99%, or 100% identical to amino acid sequence NTN, a LCDR3 at least 65%, at least 70%, at least 75%, at least 80%, at least 85%, at least 90%, at least 95%, at least 99%, or 100% identical to SEQ ID NO: 1272, a HCDR1 at least 65%, at least 70%, at least 75%, at least 80%, at least 85%, at least 90%, at least 95%, at least 99%, or 100% identical to SEQ ID NO: 1274, a HCDR2 at least 65%, at least 70%, at least 75%, at least 80%, at least 85%, at least 90%, at least 95%, at least 99%, or 100% identical to SEQ ID NO: 1275, and a HCDR3 at least 65%, at least 70%, at least 75%, at least 80%, at least 85%, at least 90%, at least 95%, at least 99%, or 100% identical to SEQ ID NO: 1276;
a LCDR1 at least 65%, at least 70%, at least 75%, at least 80%, at least 85%, at least 90%, at least 95%, at least 99%, or 100% identical to SEQ ID NO: 1278, a LCDR2 at least 65%, at least 70%, at least 75%, at least 80%, at least 85%, at least 90%, at least 95%, at least 99%, or 100% identical to amino acid sequence YAT, a LCDR3 at least 65%, at least 70%, at least 75%, at least 80%, at least 85%, at least 90%, at least 95%, at least 99%, or 100% identical to SEQ ID NO: 1279, a HCDR1 at least 65%, at least 70%, at least 75%, at least 80%, at least 85%, at least 90%, at least 95%, at least 99%, or 100% identical to SEQ ID NO: 1281, a HCDR2 at least 65%, at least 70%, at least 75%, at least 80%, at least 85%, at least 90%, at least 95%, at least 99%, or 100% identical to SEQ ID NO: 1282, and a HCDR3 at least 65%, at least 70%, at least 75%, at least 80%, at least 85%, at least 90%, at least 95%, at least 99%, or 100% identical to SEQ ID NO: 1283;
a LCDR1 at least 65%, at least 70%, at least 75%, at least 80%, at least 85%, at least 90%, at least 95%, at least 99%, or 100% identical to SEQ ID NO: 1285, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1286, a HCDR1 at least 65%, at least 70%, at least 75%, at least 80%, at least 85%, at least 90%, at least 95%, at least 99%, or 100% identical to SEQ ID NO: 1288, a HCDR2 at least 65%, at least 70%, at least 75%, at least 80%, at least 85%, at least 90%, at least 95%, at least 99%, or 100% identical to SEQ ID NO: 1289, and a HCDR3 at least 65%, at least 70%, at least 75%, at least 80%, at least 85%, at least 90%, at least 95%, at least 99%, or 100% identical to SEQ ID NO: 1290;
a LCDR1 at least 65%, at least 70%, at least 75%, at least 80%, at least 85%, at least 90%, at least 95%, at least 99%, or 100% identical to SEQ ID NO: 1292, a LCDR2 at least 65%, at least 70%, at least 75%, at least 80%, at least 85%, at least 90%, at least 95%, at least 99%, or 100% identical to amino acid sequence WAS, a LCDR3 at least 65%, at least 70%, at least 75%, at least 80%, at least 85%, at least 90%, at least 95%, at least 99%, or 100% identical to SEQ ID NO: 1293, a HCDR1 at least 65%, at least 70%, at least 75%, at least 80%, at least 85%, at least 90%, at least 95%, at least 99%, or 100% identical to SEQ ID NO: 1295, a HCDR2 at least 65%, at least 70%, at least 75%, at least 80%, at least 85%, at least 90%, at least 95%, at least 99%, or 100% identical to SEQ ID NO: 1296, and a HCDR3 at least 65%, at least 70%, at least 75%, at least 80%, at least 85%, at least 90%, at least 95%, at least 99%, or 100% identical to SEQ ID NO: 1297;
a LCDR1 at least 65%, at least 70%, at least 75%, at least 80%, at least 85%, at least 90%, at least 95%, at least 99%, or 100% identical to SEQ ID NO: 1299, a LCDR2 at least 65%, at least 70%, at least 75%, at least 80%, at least 85%, at least 90%, at least 95%, at least 99%, or 100% identical to amino acid sequence SAS, a LCDR3 at least 65%, at least 70%, at least 75%, at least 80%, at least 85%, at least 90%, at least 95%, at least 99%, or 100% identical to SEQ ID NO: 1300, a HCDR1 at least 65%, at least 70%, at least 75%, at least 80%, at least 85%, at least 90%, at least 95%, at least 99%, or 100% identical to SEQ ID NO: 1302, a HCDR2 at least 65%, at least 70%, at least 75%, at least 80%, at least 85%, at least 90%, at least 95%, at least 99%, or 100% identical to SEQ ID NO: 1303, and a HCDR3 at least 65%, at least 70%, at least 75%, at least 80%, at least 85%, at least 90%, at least 95%, at least 99%, or 100% identical to SEQ ID NO: 1304;
a LCDR1 at least 65%, at least 70%, at least 75%, at least 80%, at least 85%, at least 90%, at least 95%, at least 99%, or 100% identical to SEQ ID NO: 1306, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1307, a HCDR1 at least 65%, at least 70%, at least 75%, at least 80%, at least 85%, at least 90%, at least 95%, at least 99%, or 100% identical to SEQ ID NO: 1309, a HCDR2 at least 65%, at least 70%, at least 75%, at least 80%, at least 85%, at least 90%, at least 95%, at least 99%, or 100% identical to SEQ ID NO: 1310, and a HCDR3 at least 65%, at least 70%, at least 75%, at least 80%, at least 85%, at least 90%, at least 95%, at least 99%, or 100% identical to SEQ ID NO: 1311;
a LCDR1 at least 65%, at least 70%, at least 75%, at least 80%, at least 85%, at least 90%, at least 95%, at least 99%, or 100% identical to SEQ ID NO: 1313, a LCDR2 at least 65%, at least 70%, at least 75%, at least 80%, at least 85%, at least 90%, at least 95%, at least 99%, or 100% identical to amino acid sequence YAS, a LCDR3 at least 65%, at least 70%, at least 75%, at least 80%, at least 85%, at least 90%, at least 95%, at least 99%, or 100% identical to SEQ ID NO: 1976, a HCDR1 at least 65%, at least 70%, at least 75%, at least 80%, at least 85%, at least 90%, at least 95%, at least 99%, or 100% identical to SEQ ID NO: 1315, a HCDR2 at least 65%, at least 70%, at least 75%, at least 80%, at least 85%, at least 90%, at least 95%, at least 99%, or 100% identical to SEQ ID NO: 1316, and a HCDR3 at least 65%, at least 70%, at least 75%, at least 80%, at least 85%, at least 90%, at least 95%, at least 99%, or 100% identical to SEQ ID NO: 1317;
a LCDR1 at least 65%, at least 70%, at least 75%, at least 80%, at least 85%, at least 90%, at least 95%, at least 99%, or 100% identical to SEQ ID NO: 1319, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1320, a HCDR1 at least 65%, at least 70%, at least 75%, at least 80%, at least 85%, at least 90%, at least 95%, at least 99%, or 100% identical to SEQ ID NO: 1322, a HCDR2 at least 65%, at least 70%, at least 75%, at least 80%, at least 85%, at least 90%, at least 95%, at least 99%, or 100% identical to SEQ ID NO: 1323, and a HCDR3 at least 65%, at least 70%, at least 75%, at least 80%, at least 85%, at least 90%, at least 95%, at least 99%, or 100% identical to SEQ ID NO: 1324;
a LCDR1 at least 65%, at least 70%, at least 75%, at least 80%, at least 85%, at least 90%, at least 95%, at least 99%, or 100% identical to SEQ ID NO: 1326, a LCDR2 at least 65%, at least 70%, at least 75%, at least 80%, at least 85%, at least 90%, at least 95%, at least 99%, or 100% identical to amino acid sequence GAF, a LCDR3 at least 65%, at least 70%, at least 75%, at least 80%, at least 85%, at least 90%, at least 95%, at least 99%, or 100% identical to SEQ ID NO: 1327, a HCDR1 at least 65%, at least 70%, at least 75%, at least 80%, at least 85%, at least 90%, at least 95%, at least 99%, or 100% identical to SEQ ID NO: 1329, a HCDR2 at least 65%, at least 70%, at least 75%, at least 80%, at least 85%, at least 90%, at least 95%, at least 99%, or 100% identical to SEQ ID NO: 1330, and a HCDR3 at least 65%, at least 70%, at least 75%, at least 80%, at least 85%, at least 90%, at least 95%, at least 99%, or 100% identical to SEQ ID NO: 1331;
a LCDR1 at least 65%, at least 70%, at least 75%, at least 80%, at least 85%, at least 90%, at least 95%, at least 99%, or 100% identical to SEQ ID NO: 1228, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1333, a HCDR1 at least 65%, at least 70%, at least 75%, at least 80%, at least 85%, at least 90%, at least 95%, at least 99%, or 100% identical to SEQ ID NO: 1335, a HCDR2 at least 65%, at least 70%, at least 75%, at least 80%, at least 85%, at least 90%, at least 95%, at least 99%, or 100% identical to SEQ ID NO: 1336, and a HCDR3 at least 65%, at least 70%, at least 75%, at least 80%, at least 85%, at least 90%, at least 95%, at least 99%, or 100% identical to SEQ ID NO: 1337;
a LCDR1 at least 65%, at least 70%, at least 75%, at least 80%, at least 85%, at least 90%, at least 95%, at least 99%, or 100% identical to SEQ ID NO: 1339, a LCDR2 at least 65%, at least 70%, at least 75%, at least 80%, at least 85%, at least 90%, at least 95%, at least 99%, or 100% identical to amino acid sequence YAS, a LCDR3 at least 65%, at least 70%, at least 75%, at least 80%, at least 85%, at least 90%, at least 95%, at least 99%, or 100% identical to SEQ ID NO: 1340, a HCDR1 at least 65%, at least 70%, at least 75%, at least 80%, at least 85%, at least 90%, at least 95%, at least 99%, or 100% identical to SEQ ID NO: 1342, a HCDR2 at least 65%, at least 70%, at least 75%, at least 80%, at least 85%, at least 90%, at least 95%, at least 99%, or 100% identical to SEQ ID NO: 1343, and a HCDR3 at least 65%, at least 70%, at least 75%, at least 80%, at least 85%, at least 90%, at least 95%, at least 99%, or 100% identical to SEQ ID NO: 1344;
a LCDR1 at least 65%, at least 70%, at least 75%, at least 80%, at least 85%, at least 90%, at least 95%, at least 99%, or 100% identical to SEQ ID NO: 1188, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1346, a HCDR1 at least 65%, at least 70%, at least 75%, at least 80%, at least 85%, at least 90%, at least 95%, at least 99%, or 100% identical to SEQ ID NO: 1348, a HCDR2 at least 65%, at least 70%, at least 75%, at least 80%, at least 85%, at least 90%, at least 95%, at least 99%, or 100% identical to SEQ ID NO: 1349, and a HCDR3 at least 65%, at least 70%, at least 75%, at least 80%, at least 85%, at least 90%, at least 95%, at least 99%, or 100% identical to SEQ ID NO: 1350;
a LCDR1 at least 65%, at least 70%, at least 75%, at least 80%, at least 85%, at least 90%, at least 95%, at least 99%, or 100% identical to SEQ ID NO: 1352, a LCDR2 at least 65%, at least 70%, at least 75%, at least 80%, at least 85%, at least 90%, at least 95%, at least 99%, or 100% identical to amino acid sequence KVS, a LCDR3 at least 65%, at least 70%, at least 75%, at least 80%, at least 85%, at least 90%, at least 95%, at least 99%, or 100% identical to SEQ ID NO: 1353, a HCDR1 at least 65%, at least 70%, at least 75%, at least 80%, at least 85%, at least 90%, at least 95%, at least 99%, or 100% identical to SEQ ID NO: 1355, a HCDR2 at least 65%, at least 70%, at least 75%, at least 80%, at least 85%, at least 90%, at least 95%, at least 99%, or 100% identical to SEQ ID NO: 1356, and a HCDR3 at least 65%, at least 70%, at least 75%, at least 80%, at least 85%, at least 90%, at least 95%, at least 99%, or 100% identical to SEQ ID NO: 1357;
a LCDR1 at least 65%, at least 70%, at least 75%, at least 80%, at least 85%, at least 90%, at least 95%, at least 99%, or 100% identical to SEQ ID NO: 1359, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1360, a HCDR1 at least 65%, at least 70%, at least 75%, at least 80%, at least 85%, at least 90%, at least 95%, at least 99%, or 100% identical to SEQ ID NO: 1362, a HCDR2 at least 65%, at least 70%, at least 75%, at least 80%, at least 85%, at least 90%, at least 95%, at least 99%, or 100% identical to SEQ ID NO: 1363, and a HCDR3 at least 65%, at least 70%, at least 75%, at least 80%, at least 85%, at least 90%, at least 95%, at least 99%, or 100% identical to SEQ ID NO: 1364;
a LCDR1 at least 65%, at least 70%, at least 75%, at least 80%, at least 85%, at least 90%, at least 95%, at least 99%, or 100% identical to SEQ ID NO: 1366, a LCDR2 at least 65%, at least 70%, at least 75%, at least 80%, at least 85%, at least 90%, at least 95%, at least 99%, or 100% identical to amino acid sequence KVS, a LCDR3 at least 65%, at least 70%, at least 75%, at least 80%, at least 85%, at least 90%, at least 95%, at least 99%, or 100% identical to SEQ ID NO: 1367, a HCDR1 at least 65%, at least 70%, at least 75%, at least 80%, at least 85%, at least 90%, at least 95%, at least 99%, or 100% identical to SEQ ID NO: 1369, a HCDR2 at least 65%, at least 70%, at least 75%, at least 80%, at least 85%, at least 90%, at least 95%, at least 99%, or 100% identical to SEQ ID NO: 1370, and a HCDR3 at least 65%, at least 70%, at least 75%, at least 80%, at least 85%, at least 90%, at least 95%, at least 99%, or 100% identical to SEQ ID NO: 1371;
a LCDR1 at least 65%, at least 70%, at least 75%, at least 80%, at least 85%, at least 90%, at least 95%, at least 99%, or 100% identical to SEQ ID NO: 1366, a LCDR2 at least 65%, at least 70%, at least 75%, at least 80%, at least 85%, at least 90%, at least 95%, at least 99%, or 100% identical to amino acid sequence KVS, a LCDR3 at least 65%, at least 70%, at least 75%, at least 80%, at least 85%, at least 90%, at least 95%, at least 99%, or 100% identical to SEQ ID NO: 1367, a HCDR1 at least 65%, at least 70%, at least 75%, at least 80%, at least 85%, at least 90%, at least 95%, at least 99%, or 100% identical to SEQ ID NO: 1369, a HCDR2 at least 65%, at least 70%, at least 75%, at least 80%, at least 85%, at least 90%, at least 95%, at least 99%, or 100% identical to SEQ ID NO: 1370, and a HCDR3 at least 65%, at least 70%, at least 75%, at least 80%, at least 85%, at least 90%, at least 95%, at least 99%, or 100% identical to SEQ ID NO: 1371;
a LCDR1 at least 65%, at least 70%, at least 75%, at least 80%, at least 85%, at least 90%, at least 95%, at least 99%, or 100% identical to SEQ ID NO: 1373, a LCDR2 at least 65%, at least 70%, at least 75%, at least 80%, at least 85%, at least 90%, at least 95%, at least 99%, or 100% identical to amino acid sequence SAS, a LCDR3 at least 65%, at least 70%, at least 75%, at least 80%, at least 85%, at least 90%, at least 95%, at least 99%, or 100% identical to SEQ ID NO: 1374, a HCDR1 at least 65%, at least 70%, at least 75%, at least 80%, at least 85%, at least 90%, at least 95%, at least 99%, or 100% identical to SEQ ID NO: 1376, a HCDR2 at least 65%, at least 70%, at least 75%, at least 80%, at least 85%, at least 90%, at least 95%, at least 99%, or 100% identical to SEQ ID NO: 1377, and a HCDR3 at least 65%, at least 70%, at least 75%, at least 80%, at least 85%, at least 90%, at least 95%, at least 99%, or 100% identical to SEQ ID NO: 1378;
a LCDR1 at least 65%, at least 70%, at least 75%, at least 80%, at least 85%, at least 90%, at least 95%, at least 99%, or 100% identical to SEQ ID NO: 1373, a LCDR2 at least 65%, at least 70%, at least 75%, at least 80%, at least 85%, at least 90%, at least 95%, at least 99%, or 100% identical to amino acid sequence SAS, a LCDR3 at least 65%, at least 70%, at least 75%, at least 80%, at least 85%, at least 90%, at least 95%, at least 99%, or 100% identical to SEQ ID NO: 1374, a HCDR1 at least 65%, at least 70%, at least 75%, at least 80%, at least 85%, at least 90%, at least 95%, at least 99%, or 100% identical to SEQ ID NO: 1376, a HCDR2 at least 65%, at least 70%, at least 75%, at least 80%, at least 85%, at least 90%, at least 95%, at least 99%, or 100% identical to SEQ ID NO: 1377, and a HCDR3 at least 65%, at least 70%, at least 75%, at least 80%, at least 85%, at least 90%, at least 95%, at least 99%, or 100% identical to SEQ ID NO: 1378;
a LCDR1 at least 65%, at least 70%, at least 75%, at least 80%, at least 85%, at least 90%, at least 95%, at least 99%, or 100% identical to SEQ ID NO: 1373, a LCDR2 at least 65%, at least 70%, at least 75%, at least 80%, at least 85%, at least 90%, at least 95%, at least 99%, or 100% identical to amino acid sequence SAS, a LCDR3 at least 65%, at least 70%, at least 75%, at least 80%, at least 85%, at least 90%, at least 95%, at least 99%, or 100% identical to SEQ ID NO: 1374, a HCDR1 at least 65%, at least 70%, at least 75%, at least 80%, at least 85%, at least 90%, at least 95%, at least 99%, or 100% identical to SEQ ID NO: 1376, a HCDR2 at least 65%, at least 70%, at least 75%, at least 80%, at least 85%, at least 90%, at least 95%, at least 99%, or 100% identical to SEQ ID NO: 1377, and a HCDR3 at least 65%, at least 70%, at least 75%, at least 80%, at least 85%, at least 90%, at least 95%, at least 99%, or 100% identical to SEQ ID NO: 1378;
a LCDR1 at least 65%, at least 70%, at least 75%, at least 80%, at least 85%, at least 90%, at least 95%, at least 99%, or 100% identical to SEQ ID NO: 1373, a LCDR2 at least 65%, at least 70%, at least 75%, at least 80%, at least 85%, at least 90%, at least 95%, at least 99%, or 100% identical to amino acid sequence SAS, a LCDR3 at least 65%, at least 70%, at least 75%, at least 80%, at least 85%, at least 90%, at least 95%, at least 99%, or 100% identical to SEQ ID NO: 1374, a HCDR1 at least 65%, at least 70%, at least 75%, at least 80%, at least 85%, at least 90%, at least 95%, at least 99%, or 100% identical to SEQ ID NO: 1376, a HCDR2 at least 65%, at least 70%, at least 75%, at least 80%, at least 85%, at least 90%, at least 95%, at least 99%, or 100% identical to SEQ ID NO: 1377, and a HCDR3 at least 65%, at least 70%, at least 75%, at least 80%, at least 85%, at least 90%, at least 95%, at least 99%, or 100% identical to SEQ ID NO: 1378;
a LCDR1 at least 65%, at least 70%, at least 75%, at least 80%, at least 85%, at least 90%, at least 95%, at least 99%, or 100% identical to SEQ ID NO: 1382, a LCDR2 at least 65%, at least 70%, at least 75%, at least 80%, at least 85%, at least 90%, at least 95%, at least 99%, or 100% identical to amino acid sequence SAS, a LCDR3 at least 65%, at least 70%, at least 75%, at least 80%, at least 85%, at least 90%, at least 95%, at least 99%, or 100% identical to SEQ ID NO: 1383, a HCDR1 at least 65%, at least 70%, at least 75%, at least 80%, at least 85%, at least 90%, at least 95%, at least 99%, or 100% identical to SEQ ID NO: 1385, a HCDR2 at least 65%, at least 70%, at least 75%, at least 80%, at least 85%, at least 90%, at least 95%, at least 99%, or 100% identical to SEQ ID NO: 1386, and a HCDR3 at least 65%, at least 70%, at least 75%, at least 80%, at least 85%, at least 90%, at least 95%, at least 99%, or 100% identical to SEQ ID NO: 1387;
a LCDR1 at least 65%, at least 70%, at least 75%, at least 80%, at least 85%, at least 90%, at least 95%, at least 99%, or 100% identical to SEQ ID NO: 1188, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1389, a HCDR1 at least 65%, at least 70%, at least 75%, at least 80%, at least 85%, at least 90%, at least 95%, at least 99%, or 100% identical to SEQ ID NO: 1322, a HCDR2 at least 65%, at least 70%, at least 75%, at least 80%, at least 85%, at least 90%, at least 95%, at least 99%, or 100% identical to SEQ ID NO: 1391, and a HCDR3 at least 65%, at least 70%, at least 75%, at least 80%, at least 85%, at least 90%, at least 95%, at least 99%, or 100% identical to SEQ ID NO: 1392;
a LCDR1 at least 65%, at least 70%, at least 75%, at least 80%, at least 85%, at least 90%, at least 95%, at least 99%, or 100% identical to SEQ ID NO: 1394, a LCDR2 at least 65%, at least 70%, at least 75%, at least 80%, at least 85%, at least 90%, at least 95%, at least 99%, or 100% identical to amino acid sequence YAS, a LCDR3 at least 65%, at least 70%, at least 75%, at least 80%, at least 85%, at least 90%, at least 95%, at least 99%, or 100% identical to SEQ ID NO: 1395, a HCDR1 at least 65%, at least 70%, at least 75%, at least 80%, at least 85%, at least 90%, at least 95%, at least 99%, or 100% identical to SEQ ID NO: 1397, a HCDR2 at least 65%, at least 70%, at least 75%, at least 80%, at least 85%, at least 90%, at least 95%, at least 99%, or 100% identical to SEQ ID NO: 1398, and a HCDR3 at least 65%, at least 70%, at least 75%, at least 80%, at least 85%, at least 90%, at least 95%, at least 99%, or 100% identical to SEQ ID NO: 1399;
a LCDR1 at least 65%, at least 70%, at least 75%, at least 80%, at least 85%, at least 90%, at least 95%, at least 99%, or 100% identical to SEQ ID NO: 1401, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1402, a HCDR1 at least 65%, at least 70%, at least 75%, at least 80%, at least 85%, at least 90%, at least 95%, at least 99%, or 100% identical to SEQ ID NO: 1404, a HCDR2 at least 65%, at least 70%, at least 75%, at least 80%, at least 85%, at least 90%, at least 95%, at least 99%, or 100% identical to SEQ ID NO: 1405, and a HCDR3 at least 65%, at least 70%, at least 75%, at least 80%, at least 85%, at least 90%, at least 95%, at least 99%, or 100% identical to SEQ ID NO: 1406;
a LCDR1 at least 65%, at least 70%, at least 75%, at least 80%, at least 85%, at least 90%, at least 95%, at least 99%, or 100% identical to SEQ ID NO: 1408, a LCDR2 at least 65%, at least 70%, at least 75%, at least 80%, at least 85%, at least 90%, at least 95%, at least 99%, or 100% identical to amino acid sequence DDG, a LCDR3 at least 65%, at least 70%, at least 75%, at least 80%, at least 85%, at least 90%, at least 95%, at least 99%, or 100% identical to SEQ ID NO: 1409, a HCDR1 at least 65%, at least 70%, at least 75%, at least 80%, at least 85%, at least 90%, at least 95%, at least 99%, or 100% identical to SEQ ID NO: 1203, a HCDR2 at least 65%, at least 70%, at least 75%, at least 80%, at least 85%, at least 90%, at least 95%, at least 99%, or 100% identical to SEQ ID NO: 1411, and a HCDR3 at least 65%, at least 70%, at least 75%, at least 80%, at least 85%, at least 90%, at least 95%, at least 99%, or 100% identical to SEQ ID NO: 1412;
a LCDR1 at least 65%, at least 70%, at least 75%, at least 80%, at least 85%, at least 90%, at least 95%, at least 99%, or 100% identical to SEQ ID NO: 1414, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1415, a HCDR1 at least 65%, at least 70%, at least 75%, at least 80%, at least 85%, at least 90%, at least 95%, at least 99%, or 100% identical to SEQ ID NO: 1417, a HCDR2 at least 65%, at least 70%, at least 75%, at least 80%, at least 85%, at least 90%, at least 95%, at least 99%, or 100% identical to SEQ ID NO: 1418, and a HCDR3 at least 65%, at least 70%, at least 75%, at least 80%, at least 85%, at least 90%, at least 95%, at least 99%, or 100% identical to SEQ ID NO: 1419;
a LCDR1 at least 65%, at least 70%, at least 75%, at least 80%, at least 85%, at least 90%, at least 95%, at least 99%, or 100% identical to SEQ ID NO: 1421, a LCDR2 at least 65%, at least 70%, at least 75%, at least 80%, at least 85%, at least 90%, at least 95%, at least 99%, or 100% identical to amino acid sequence KVS, a LCDR3 at least 65%, at least 70%, at least 75%, at least 80%, at least 85%, at least 90%, at least 95%, at least 99%, or 100% identical to SEQ ID NO: 1422, a HCDR1 at least 65%, at least 70%, at least 75%, at least 80%, at least 85%, at least 90%, at least 95%, at least 99%, or 100% identical to SEQ ID NO: 1424, a HCDR2 at least 65%, at least 70%, at least 75%, at least 80%, at least 85%, at least 90%, at least 95%, at least 99%, or 100% identical to SEQ ID NO: 1425, and a HCDR3 at least 65%, at least 70%, at least 75%, at least 80%, at least 85%, at least 90%, at least 95%, at least 99%, or 100% identical to SEQ ID NO: 1426;
a LCDR1 at least 65%, at least 70%, at least 75%, at least 80%, at least 85%, at least 90%, at least 95%, at least 99%, or 100% identical to SEQ ID NO: 1428, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1429, a HCDR1 at least 65%, at least 70%, at least 75%, at least 80%, at least 85%, at least 90%, at least 95%, at least 99%, or 100% identical to SEQ ID NO: 1431, a HCDR2 at least 65%, at least 70%, at least 75%, at least 80%, at least 85%, at least 90%, at least 95%, at least 99%, or 100% identical to SEQ ID NO: 1432, and a HCDR3 at least 65%, at least 70%, at least 75%, at least 80%, at least 85%, at least 90%, at least 95%, at least 99%, or 100% identical to SEQ ID NO: 1433;
a LCDR1 at least 65%, at least 70%, at least 75%, at least 80%, at least 85%, at least 90%, at least 95%, at least 99%, or 100% identical to SEQ ID NO: 1428, a LCDR2 at least 65%, at least 70%, at least 75%, at least 80%, at least 85%, at least 90%, at least 95%, at least 99%, or 100% identical to amino acid sequence GAS, a LCDR3 at least 65%, at least 70%, at least 75%, at least 80%, at least 85%, at least 90%, at least 95%, at least 99%, or 100% identical to SEQ ID NO: 1435, a HCDR1 at least 65%, at least 70%, at least 75%, at least 80%, at least 85%, at least 90%, at least 95%, at least 99%, or 100% identical to SEQ ID NO: 1437, a HCDR2 at least 65%, at least 70%, at least 75%, at least 80%, at least 85%, at least 90%, at least 95%, at least 99%, or 100% identical to SEQ ID NO: 1438, and a HCDR3 at least 65%, at least 70%, at least 75%, at least 80%, at least 85%, at least 90%, at least 95%, at least 99%, or 100% identical to SEQ ID NO: 1439;
a LCDR1 at least 65%, at least 70%, at least 75%, at least 80%, at least 85%, at least 90%, at least 95%, at least 99%, or 100% identical to SEQ ID NO: 1441, a LCDR2 at least 65%, at least 70%, at least 75%, at least 80%, at least 85%, at least 90%, at least 95%, at least 99%, or 100% identical to amino acid sequence LVS, a LCDR3 at least 65%, at least 70%, at least 75%, at least 80%, at least 85%, at least 90%, at least 95%, at least 99%, or 100% identical to SEQ ID NO: 1442, a HCDR1 at least 65%, at least 70%, at least 75%, at least 80%, at least 85%, at least 90%, at least 95%, at least 99%, or 100% identical to SEQ ID NO: 1444, a HCDR2 at least 65%, at least 70%, at least 75%, at least 80%, at least 85%, at least 90%, at least 95%, at least 99%, or 100% identical to SEQ ID NO: 1445, and a HCDR3 at least 65%, at least 70%, at least 75%, at least 80%, at least 85%, at least 90%, at least 95%, at least 99%, or 100% identical to SEQ ID NO: 1446;
a LCDR1 at least 65%, at least 70%, at least 75%, at least 80%, at least 85%, at least 90%, at least 95%, at least 99%, or 100% identical to SEQ ID NO: 1448, a LCDR2 at least 65%, at least 70%, at least 75%, at least 80%, at least 85%, at least 90%, at least 95%, at least 99%, or 100% identical to amino acid sequence GNS, a LCDR3 at least 65%, at least 70%, at least 75%, at least 80%, at least 85%, at least 90%, at least 95%, at least 99%, or 100% identical to SEQ ID NO: 1449, a HCDR1 at least 65%, at least 70%, at least 75%, at least 80%, at least 85%, at least 90%, at least 95%, at least 99%, or 100% identical to SEQ ID NO: 1451, a HCDR2 at least 65%, at least 70%, at least 75%, at least 80%, at least 85%, at least 90%, at least 95%, at least 99%, or 100% identical to SEQ ID NO: 1452, and a HCDR3 at least 65%, at least 70%, at least 75%, at least 80%, at least 85%, at least 90%, at least 95%, at least 99%, or 100% identical to SEQ ID NO: 1453;
a LCDR1 at least 65%, at least 70%, at least 75%, at least 80%, at least 85%, at least 90%, at least 95%, at least 99%, or 100% identical to SEQ ID NO: 1455, a LCDR2 at least 65%, at least 70%, at least 75%, at least 80%, at least 85%, at least 90%, at least 95%, at least 99%, or 100% identical to amino acid sequence GAT, a LCDR3 at least 65%, at least 70%, at least 75%, at least 80%, at least 85%, at least 90%, at least 95%, at least 99%, or 100% identical to SEQ ID NO: 1456, a HCDR1 at least 65%, at least 70%, at least 75%, at least 80%, at least 85%, at least 90%, at least 95%, at least 99%, or 100% identical to SEQ ID NO: 1458, a HCDR2 at least 65%, at least 70%, at least 75%, at least 80%, at least 85%, at least 90%, at least 95%, at least 99%, or 100% identical to SEQ ID NO: 1145, and a HCDR3 at least 65%, at least 70%, at least 75%, at least 80%, at least 85%, at least 90%, at least 95%, at least 99%, or 100% identical to SEQ ID NO: 1459;
a LCDR1 at least 65%, at least 70%, at least 75%, at least 80%, at least 85%, at least 90%, at least 95%, at least 99%, or 100% identical to SEQ ID NO: 1461, a LCDR2 at least 65%, at least 70%, at least 75%, at least 80%, at least 85%, at least 90%, at least 95%, at least 99%, or 100% identical to amino acid sequence NAK, a LCDR3 at least 65%, at least 70%, at least 75%, at least 80%, at least 85%, at least 90%, at least 95%, at least 99%, or 100% identical to SEQ ID NO: 1462, a HCDR1 at least 65%, at least 70%, at least 75%, at least 80%, at least 85%, at least 90%, at least 95%, at least 99%, or 100% identical to SEQ ID NO: 1464, a HCDR2 at least 65%, at least 70%, at least 75%, at least 80%, at least 85%, at least 90%, at least 95%, at least 99%, or 100% identical to SEQ ID NO: 1465, and a HCDR3 at least 65%, at least 70%, at least 75%, at least 80%, at least 85%, at least 90%, at least 95%, at least 99%, or 100% identical to SEQ ID NO: 1466;
a LCDR1 at least 65%, at least 70%, at least 75%, at least 80%, at least 85%, at least 90%, at least 95%, at least 99%, or 100% identical to SEQ ID NO: 1468, a LCDR2 at least 65%, at least 70%, at least 75%, at least 80%, at least 85%, at least 90%, at least 95%, at least 99%, or 100% identical to amino acid sequence WAS, a LCDR3 at least 65%, at least 70%, at least 75%, at least 80%, at least 85%, at least 90%, at least 95%, at least 99%, or 100% identical to SEQ ID NO: 1469, a HCDR1 at least 65%, at least 70%, at least 75%, at least 80%, at least 85%, at least 90%, at least 95%, at least 99%, or 100% identical to SEQ ID NO: 1471, a HCDR2 at least 65%, at least 70%, at least 75%, at least 80%, at least 85%, at least 90%, at least 95%, at least 99%, or 100% identical to SEQ ID NO: 1472, and a HCDR3 at least 65%, at least 70%, at least 75%, at least 80%, at least 85%, at least 90%, at least 95%, at least 99%, or 100% identical to SEQ ID NO: 1473;
a LCDR1 at least 65%, at least 70%, at least 75%, at least 80%, at least 85%, at least 90%, at least 95%, at least 99%, or 100% identical to SEQ ID NO: 1475, a LCDR2 at least 65%, at least 70%, at least 75%, at least 80%, at least 85%, at least 90%, at least 95%, at least 99%, or 100% identical to amino acid sequence LGS, a LCDR3 at least 65%, at least 70%, at least 75%, at least 80%, at least 85%, at least 90%, at least 95%, at least 99%, or 100% identical to SEQ ID NO: 1476, a HCDR1 at least 65%, at least 70%, at least 75%, at least 80%, at least 85%, at least 90%, at least 95%, at least 99%, or 100% identical to SEQ ID NO: 1478, a HCDR2 at least 65%, at least 70%, at least 75%, at least 80%, at least 85%, at least 90%, at least 95%, at least 99%, or 100% identical to SEQ ID NO: 1479, and a HCDR3 at least 65%, at least 70%, at least 75%, at least 80%, at least 85%, at least 90%, at least 95%, at least 99%, or 100% identical to SEQ ID NO: 1480;
a LCDR1 at least 65%, at least 70%, at least 75%, at least 80%, at least 85%, at least 90%, at least 95%, at least 99%, or 100% identical to SEQ ID NO: 1292, a LCDR2 at least 65%, at least 70%, at least 75%, at least 80%, at least 85%, at least 90%, at least 95%, at least 99%, or 100% identical to amino acid sequence GAS, a LCDR3 at least 65%, at least 70%, at least 75%, at least 80%, at least 85%, at least 90%, at least 95%, at least 99%, or 100% identical to SEQ ID NO: 1482, a HCDR1 at least 65%, at least 70%, at least 75%, at least 80%, at least 85%, at least 90%, at least 95%, at least 99%, or 100% identical to SEQ ID NO: 1484, a HCDR2 at least 65%, at least 70%, at least 75%, at least 80%, at least 85%, at least 90%, at least 95%, at least 99%, or 100% identical to SEQ ID NO: 1485, and a HCDR3 at least 65%, at least 70%, at least 75%, at least 80%, at least 85%, at least 90%, at least 95%, at least 99%, or 100% identical to SEQ ID NO: 1486;
a LCDR1 at least 65%, at least 70%, at least 75%, at least 80%, at least 85%, at least 90%, at least 95%, at least 99%, or 100% identical to SEQ ID NO: 1488, a LCDR2 at least 65%, at least 70%, at least 75%, at least 80%, at least 85%, at least 90%, at least 95%, at least 99%, or 100% identical to amino acid sequence GAN, a LCDR3 at least 65%, at least 70%, at least 75%, at least 80%, at least 85%, at least 90%, at least 95%, at least 99%, or 100% identical to SEQ ID NO: 1489, a HCDR1 at least 65%, at least 70%, at least 75%, at least 80%, at least 85%, at least 90%, at least 95%, at least 99%, or 100% identical to SEQ ID NO: 1491, a HCDR2 at least 65%, at least 70%, at least 75%, at least 80%, at least 85%, at least 90%, at least 95%, at least 99%, or 100% identical to SEQ ID NO: 1492, and a HCDR3 at least 65%, at least 70%, at least 75%, at least 80%, at least 85%, at least 90%, at least 95%, at least 99%, or 100% identical to SEQ ID NO: 1493;
a LCDR1 at least 65%, at least 70%, at least 75%, at least 80%, at least 85%, at least 90%, at least 95%, at least 99%, or 100% identical to SEQ ID NO: 1495, a LCDR2 at least 65%, at least 70%, at least 75%, at least 80%, at least 85%, at least 90%, at least 95%, at least 99%, or 100% identical to amino acid sequence YTS, a LCDR3 at least 65%, at least 70%, at least 75%, at least 80%, at least 85%, at least 90%, at least 95%, at least 99%, or 100% identical to SEQ ID NO: 1496, a HCDR1 at least 65%, at least 70%, at least 75%, at least 80%, at least 85%, at least 90%, at least 95%, at least 99%, or 100% identical to SEQ ID NO: 1498, a HCDR2 at least 65%, at least 70%, at least 75%, at least 80%, at least 85%, at least 90%, at least 95%, at least 99%, or 100% identical to SEQ ID NO: 1499, and a HCDR3 at least 65%, at least 70%, at least 75%, at least 80%, at least 85%, at least 90%, at least 95%, at least 99%, or 100% identical to SEQ ID NO: 1500;
a LCDR1 at least 65%, at least 70%, at least 75%, at least 80%, at least 85%, at least 90%, at least 95%, at least 99%, or 100% identical to SEQ ID NO: 1401, a LCDR2 at least 65%, at least 70%, at least 75%, at least 80%, at least 85%, at least 90%, at least 95%, at least 99%, or 100% identical to amino acid sequence GAS, a LCDR3 at least 65%, at least 70%, at least 75%, at least 80%, at least 85%, at least 90%, at least 95%, at least 99%, or 100% identical to SEQ ID NO: 1502, a HCDR1 at least 65%, at least 70%, at least 75%, at least 80%, at least 85%, at least 90%, at least 95%, at least 99%, or 100% identical to SEQ ID NO: 1504, a HCDR2 at least 65%, at least 70%, at least 75%, at least 80%, at least 85%, at least 90%, at least 95%, at least 99%, or 100% identical to SEQ ID NO: 1505, and a HCDR3 at least 65%, at least 70%, at least 75%, at least 80%, at least 85%, at least 90%, at least 95%, at least 99%, or 100% identical to SEQ ID NO: 1506;
a LCDR1 at least 65%, at least 70%, at least 75%, at least 80%, at least 85%, at least 90%, at least 95%, at least 99%, or 100% identical to SEQ ID NO: 1508, a LCDR2 at least 65%, at least 70%, at least 75%, at least 80%, at least 85%, at least 90%, at least 95%, at least 99%, or 100% identical to amino acid sequence YGS, a LCDR3 at least 65%, at least 70%, at least 75%, at least 80%, at least 85%, at least 90%, at least 95%, at least 99%, or 100% identical to SEQ ID NO: 1509, a HCDR1 at least 65%, at least 70%, at least 75%, at least 80%, at least 85%, at least 90%, at least 95%, at least 99%, or 100% identical to SEQ ID NO: 1511, a HCDR2 at least 65%, at least 70%, at least 75%, at least 80%, at least 85%, at least 90%, at least 95%, at least 99%, or 100% identical to SEQ ID NO: 1499, and a HCDR3 at least 65%, at least 70%, at least 75%, at least 80%, at least 85%, at least 90%, at least 95%, at least 99%, or 100% identical to SEQ ID NO: 1500;
a LCDR1 at least 65%, at least 70%, at least 75%, at least 80%, at least 85%, at least 90%, at least 95%, at least 99%, or 100% identical to SEQ ID NO: 1181, a LCDR2 at least 65%, at least 70%, at least 75%, at least 80%, at least 85%, at least 90%, at least 95%, at least 99%, or 100% identical to amino acid sequence DTS, a LCDR3 at least 65%, at least 70%, at least 75%, at least 80%, at least 85%, at least 90%, at least 95%, at least 99%, or 100% identical to SEQ ID NO: 1513, a HCDR1 at least 65%, at least 70%, at least 75%, at least 80%, at least 85%, at least 90%, at least 95%, at least 99%, or 100% identical to SEQ ID NO: 1515, a HCDR2 at least 65%, at least 70%, at least 75%, at least 80%, at least 85%, at least 90%, at least 95%, at least 99%, or 100% identical to SEQ ID NO: 1516, and a HCDR3 at least 65%, at least 70%, at least 75%, at least 80%, at least 85%, at least 90%, at least 95%, at least 99%, or 100% identical to SEQ ID NO: 1517;
a LCDR1 at least 65%, at least 70%, at least 75%, at least 80%, at least 85%, at least 90%, at least 95%, at least 99%, or 100% identical to SEQ ID NO: 1519, a LCDR2 at least 65%, at least 70%, at least 75%, at least 80%, at least 85%, at least 90%, at least 95%, at least 99%, or 100% identical to amino acid sequence RTS, a LCDR3 at least 65%, at least 70%, at least 75%, at least 80%, at least 85%, at least 90%, at least 95%, at least 99%, or 100% identical to SEQ ID NO: 1520, a HCDR1 at least 65%, at least 70%, at least 75%, at least 80%, at least 85%, at least 90%, at least 95%, at least 99%, or 100% identical to SEQ ID NO: 1522, a HCDR2 at least 65%, at least 70%, at least 75%, at least 80%, at least 85%, at least 90%, at least 95%, at least 99%, or 100% identical to SEQ ID NO: 1523, and a HCDR3 at least 65%, at least 70%, at least 75%, at least 80%, at least 85%, at least 90%, at least 95%, at least 99%, or 100% identical to SEQ ID NO: 1524;
a LCDR1 at least 65%, at least 70%, at least 75%, at least 80%, at least 85%, at least 90%, at least 95%, at least 99%, or 100% identical to SEQ ID NO: 1526, a LCDR2 at least 65%, at least 70%, at least 75%, at least 80%, at least 85%, at least 90%, at least 95%, at least 99%, or 100% identical to amino acid sequence GIS, a LCDR3 at least 65%, at least 70%, at least 75%, at least 80%, at least 85%, at least 90%, at least 95%, at least 99%, or 100% identical to SEQ ID NO: 1222, a HCDR1 at least 65%, at least 70%, at least 75%, at least 80%, at least 85%, at least 90%, at least 95%, at least 99%, or 100% identical to SEQ ID NO: 1295, a HCDR2 at least 65%, at least 70%, at least 75%, at least 80%, at least 85%, at least 90%, at least 95%, at least 99%, or 100% identical to SEQ ID NO: 1528, and a HCDR3 at least 65%, at least 70%, at least 75%, at least 80%, at least 85%, at least 90%, at least 95%, at least 99%, or 100% identical to SEQ ID NO: 1529;
a LCDR1 at least 65%, at least 70%, at least 75%, at least 80%, at least 85%, at least 90%, at least 95%, at least 99%, or 100% identical to SEQ ID NO: 1531, a LCDR2 at least 65%, at least 70%, at least 75%, at least 80%, at least 85%, at least 90%, at least 95%, at least 99%, or 100% identical to amino acid sequence YRS, a LCDR3 at least 65%, at least 70%, at least 75%, at least 80%, at least 85%, at least 90%, at least 95%, at least 99%, or 100% identical to SEQ ID NO: 1532, a HCDR1 at least 65%, at least 70%, at least 75%, at least 80%, at least 85%, at least 90%, at least 95%, at least 99%, or 100% identical to SEQ ID NO: 1534, a HCDR2 at least 65%, at least 70%, at least 75%, at least 80%, at least 85%, at least 90%, at least 95%, at least 99%, or 100% identical to SEQ ID NO: 1535, and a HCDR3 at least 65%, at least 70%, at least 75%, at least 80%, at least 85%, at least 90%, at least 95%, at least 99%, or 100% identical to SEQ ID NO: 1536;
a LCDR1 at least 65%, at least 70%, at least 75%, at least 80%, at least 85%, at least 90%, at least 95%, at least 99%, or 100% identical to SEQ ID NO: 1538, a LCDR2 at least 65%, at least 70%, at least 75%, at least 80%, at least 85%, at least 90%, at least 95%, at least 99%, or 100% identical to amino acid sequence YTS, a LCDR3 at least 65%, at least 70%, at least 75%, at least 80%, at least 85%, at least 90%, at least 95%, at least 99%, or 100% identical to SEQ ID NO: 1539, a HCDR1 at least 65%, at least 70%, at least 75%, at least 80%, at least 85%, at least 90%, at least 95%, at least 99%, or 100% identical to SEQ ID NO: 1376, a HCDR2 at least 65%, at least 70%, at least 75%, at least 80%, at least 85%, at least 90%, at least 95%, at least 99%, or 100% identical to SEQ ID NO: 1541, and a HCDR3 at least 65%, at least 70%, at least 75%, at least 80%, at least 85%, at least 90%, at least 95%, at least 99%, or 100% identical to SEQ ID NO: 1542;
a LCDR1 at least 65%, at least 70%, at least 75%, at least 80%, at least 85%, at least 90%, at least 95%, at least 99%, or 100% identical to SEQ ID NO: 1544, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1545, a HCDR1 at least 65%, at least 70%, at least 75%, at least 80%, at least 85%, at least 90%, at least 95%, at least 99%, or 100% identical to SEQ ID NO: 1547, a HCDR2 at least 65%, at least 70%, at least 75%, at least 80%, at least 85%, at least 90%, at least 95%, at least 99%, or 100% identical to SEQ ID NO: 1548, and a HCDR3 at least 65%, at least 70%, at least 75%, at least 80%, at least 85%, at least 90%, at least 95%, at least 99%, or 100% identical to SEQ ID NO: 1549;
a LCDR1 at least 65%, at least 70%, at least 75%, at least 80%, at least 85%, at least 90%, at least 95%, at least 99%, or 100% identical to SEQ ID NO: 1228, a LCDR2 at least 65%, at least 70%, at least 75%, at least 80%, at least 85%, at least 90%, at least 95%, at least 99%, or 100% identical to amino acid sequence YTS, a LCDR3 at least 65%, at least 70%, at least 75%, at least 80%, at least 85%, at least 90%, at least 95%, at least 99%, or 100% identical to SEQ ID NO: 1229, a HCDR1 at least 65%, at least 70%, at least 75%, at least 80%, at least 85%, at least 90%, at least 95%, at least 99%, or 100% identical to SEQ ID NO: 1552, a HCDR2 at least 65%, at least 70%, at least 75%, at least 80%, at least 85%, at least 90%, at least 95%, at least 99%, or 100% identical to SEQ ID NO: 1553, and a HCDR3 at least 65%, at least 70%, at least 75%, at least 80%, at least 85%, at least 90%, at least 95%, at least 99%, or 100% identical to SEQ ID NO: 1554;
a LCDR1 at least 65%, at least 70%, at least 75%, at least 80%, at least 85%, at least 90%, at least 95%, at least 99%, or 100% identical to SEQ ID NO: 1556, a LCDR2 at least 65%, at least 70%, at least 75%, at least 80%, at least 85%, at least 90%, at least 95%, at least 99%, or 100% identical to amino acid sequence WAS, a LCDR3 at least 65%, at least 70%, at least 75%, at least 80%, at least 85%, at least 90%, at least 95%, at least 99%, or 100% identical to SEQ ID NO: 1557, a HCDR1 at least 65%, at least 70%, at least 75%, at least 80%, at least 85%, at least 90%, at least 95%, at least 99%, or 100% identical to SEQ ID NO: 1534, a HCDR2 at least 65%, at least 70%, at least 75%, at least 80%, at least 85%, at least 90%, at least 95%, at least 99%, or 100% identical to SEQ ID NO: 1559, and a HCDR3 at least 65%, at least 70%, at least 75%, at least 80%, at least 85%, at least 90%, at least 95%, at least 99%, or 100% identical to SEQ ID NO: 1560;
a LCDR1 at least 65%, at least 70%, at least 75%, at least 80%, at least 85%, at least 90%, at least 95%, at least 99%, or 100% identical to SEQ ID NO: 1188, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1562, a HCDR1 at least 65%, at least 70%, at least 75%, at least 80%, at least 85%, at least 90%, at least 95%, at least 99%, or 100% identical to SEQ ID NO: 1564, a HCDR2 at least 65%, at least 70%, at least 75%, at least 80%, at least 85%, at least 90%, at least 95%, at least 99%, or 100% identical to SEQ ID NO: 1565, and a HCDR3 at least 65%, at least 70%, at least 75%, at least 80%, at least 85%, at least 90%, at least 95%, at least 99%, or 100% identical to SEQ ID NO: 1566;
a LCDR1 at least 65%, at least 70%, at least 75%, at least 80%, at least 85%, at least 90%, at least 95%, at least 99%, or 100% identical to SEQ ID NO: 1568, a LCDR2 at least 65%, at least 70%, at least 75%, at least 80%, at least 85%, at least 90%, at least 95%, at least 99%, or 100% identical to amino acid sequence LAS, a LCDR3 at least 65%, at least 70%, at least 75%, at least 80%, at least 85%, at least 90%, at least 95%, at least 99%, or 100% identical to SEQ ID NO: 1569, a HCDR1 at least 65%, at least 70%, at least 75%, at least 80%, at least 85%, at least 90%, at least 95%, at least 99%, or 100% identical to SEQ ID NO: 1355, a HCDR2 at least 65%, at least 70%, at least 75%, at least 80%, at least 85%, at least 90%, at least 95%, at least 99%, or 100% identical to SEQ ID NO: 1571, and a HCDR3 at least 65%, at least 70%, at least 75%, at least 80%, at least 85%, at least 90%, at least 95%, at least 99%, or 100% identical to SEQ ID NO: 1572;
a LCDR1 at least 65%, at least 70%, at least 75%, at least 80%, at least 85%, at least 90%, at least 95%, at least 99%, or 100% identical to SEQ ID NO: 1574, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1575, a HCDR1 at least 65%, at least 70%, at least 75%, at least 80%, at least 85%, at least 90%, at least 95%, at least 99%, or 100% identical to SEQ ID NO: 1577, a HCDR2 at least 65%, at least 70%, at least 75%, at least 80%, at least 85%, at least 90%, at least 95%, at least 99%, or 100% identical to SEQ ID NO: 1578, and a HCDR3 at least 65%, at least 70%, at least 75%, at least 80%, at least 85%, at least 90%, at least 95%, at least 99%, or 100% identical to SEQ ID NO: 1579;
a LCDR1 at least 65%, at least 70%, at least 75%, at least 80%, at least 85%, at least 90%, at least 95%, at least 99%, or 100% identical to SEQ ID NO: 1581, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1582, a HCDR1 at least 65%, at least 70%, at least 75%, at least 80%, at least 85%, at least 90%, at least 95%, at least 99%, or 100% identical to SEQ ID NO: 1584, a HCDR2 at least 65%, at least 70%, at least 75%, at least 80%, at least 85%, at least 90%, at least 95%, at least 99%, or 100% identical to SEQ ID NO: 1585, and a HCDR3 at least 65%, at least 70%, at least 75%, at least 80%, at least 85%, at least 90%, at least 95%, at least 99%, or 100% identical to SEQ ID NO: 1586;
a LCDR1 at least 65%, at least 70%, at least 75%, at least 80%, at least 85%, at least 90%, at least 95%, at least 99%, or 100% identical to SEQ ID NO: 1188, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1588, a HCDR1 at least 65%, at least 70%, at least 75%, at least 80%, at least 85%, at least 90%, at least 95%, at least 99%, or 100% identical to SEQ ID NO: 1590, a HCDR2 at least 65%, at least 70%, at least 75%, at least 80%, at least 85%, at least 90%, at least 95%, at least 99%, or 100% identical to SEQ ID NO: 1591, and a HCDR3 at least 65%, at least 70%, at least 75%, at least 80%, at least 85%, at least 90%, at least 95%, at least 99%, or 100% identical to SEQ ID NO: 1592;
a LCDR1 at least 65%, at least 70%, at least 75%, at least 80%, at least 85%, at least 90%, at least 95%, at least 99%, or 100% identical to SEQ ID NO: 1594, a LCDR2 at least 65%, at least 70%, at least 75%, at least 80%, at least 85%, at least 90%, at least 95%, at least 99%, or 100% identical to amino acid sequence SAS, a LCDR3 at least 65%, at least 70%, at least 75%, at least 80%, at least 85%, at least 90%, at least 95%, at least 99%, or 100% identical to SEQ ID NO: 1595, a HCDR1 at least 65%, at least 70%, at least 75%, at least 80%, at least 85%, at least 90%, at least 95%, at least 99%, or 100% identical to SEQ ID NO: 1597, a HCDR2 at least 65%, at least 70%, at least 75%, at least 80%, at least 85%, at least 90%, at least 95%, at least 99%, or 100% identical to SEQ ID NO: 1598, and a HCDR3 at least 65%, at least 70%, at least 75%, at least 80%, at least 85%, at least 90%, at least 95%, at least 99%, or 100% identical to SEQ ID NO: 1599;
a LCDR1 at least 65%, at least 70%, at least 75%, at least 80%, at least 85%, at least 90%, at least 95%, at least 99%, or 100% identical to SEQ ID NO: 1601, a LCDR2 at least 65%, at least 70%, at least 75%, at least 80%, at least 85%, at least 90%, at least 95%, at least 99%, or 100% identical to amino acid sequence DVS, a LCDR3 at least 65%, at least 70%, at least 75%, at least 80%, at least 85%, at least 90%, at least 95%, at least 99%, or 100% identical to SEQ ID NO: 1602, a HCDR1 at least 65%, at least 70%, at least 75%, at least 80%, at least 85%, at least 90%, at least 95%, at least 99%, or 100% identical to SEQ ID NO: 1604, a HCDR2 at least 65%, at least 70%, at least 75%, at least 80%, at least 85%, at least 90%, at least 95%, at least 99%, or 100% identical to SEQ ID NO: 1605, and a HCDR3 at least 65%, at least 70%, at least 75%, at least 80%, at least 85%, at least 90%, at least 95%, at least 99%, or 100% identical to SEQ ID NO: 1606;
a LCDR1 at least 65%, at least 70%, at least 75%, at least 80%, at least 85%, at least 90%, at least 95%, at least 99%, or 100% identical to SEQ ID NO: 1608, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1609, a HCDR1 at least 65%, at least 70%, at least 75%, at least 80%, at least 85%, at least 90%, at least 95%, at least 99%, or 100% identical to SEQ ID NO: 1611, a HCDR2 at least 65%, at least 70%, at least 75%, at least 80%, at least 85%, at least 90%, at least 95%, at least 99%, or 100% identical to SEQ ID NO: 1612, and a HCDR3 at least 65%, at least 70%, at least 75%, at least 80%, at least 85%, at least 90%, at least 95%, at least 99%, or 100% identical to SEQ ID NO: 1613;
a LCDR1 at least 65%, at least 70%, at least 75%, at least 80%, at least 85%, at least 90%, at least 95%, at least 99%, or 100% identical to SEQ ID NO: 1615, a LCDR2 at least 65%, at least 70%, at least 75%, at least 80%, at least 85%, at least 90%, at least 95%, at least 99%, or 100% identical to amino acid sequence GAS, a LCDR3 at least 65%, at least 70%, at least 75%, at least 80%, at least 85%, at least 90%, at least 95%, at least 99%, or 100% identical to SEQ ID NO: 1616, a HCDR1 at least 65%, at least 70%, at least 75%, at least 80%, at least 85%, at least 90%, at least 95%, at least 99%, or 100% identical to SEQ ID NO: 1618, a HCDR2 at least 65%, at least 70%, at least 75%, at least 80%, at least 85%, at least 90%, at least 95%, at least 99%, or 100% identical to SEQ ID NO: 1619, and a HCDR3 at least 65%, at least 70%, at least 75%, at least 80%, at least 85%, at least 90%, at least 95%, at least 99%, or 100% identical to SEQ ID NO: 1620;
a LCDR1 at least 65%, at least 70%, at least 75%, at least 80%, at least 85%, at least 90%, at least 95%, at least 99%, or 100% identical to SEQ ID NO: 1622, a LCDR2 at least 65%, at least 70%, at least 75%, at least 80%, at least 85%, at least 90%, at least 95%, at least 99%, or 100% identical to amino acid sequence WAS, a LCDR3 at least 65%, at least 70%, at least 75%, at least 80%, at least 85%, at least 90%, at least 95%, at least 99%, or 100% identical to SEQ ID NO: 1623, a HCDR1 at least 65%, at least 70%, at least 75%, at least 80%, at least 85%, at least 90%, at least 95%, at least 99%, or 100% identical to SEQ ID NO: 1625, a HCDR2 at least 65%, at least 70%, at least 75%, at least 80%, at least 85%, at least 90%, at least 95%, at least 99%, or 100% identical to SEQ ID NO: 1626, and a HCDR3 at least 65%, at least 70%, at least 75%, at least 80%, at least 85%, at least 90%, at least 95%, at least 99%, or 100% identical to SEQ ID NO: 1627;
a LCDR1 at least 65%, at least 70%, at least 75%, at least 80%, at least 85%, at least 90%, at least 95%, at least 99%, or 100% identical to SEQ ID NO: 1629, a LCDR2 at least 65%, at least 70%, at least 75%, at least 80%, at least 85%, at least 90%, at least 95%, at least 99%, or 100% identical to amino acid sequence YAS, a LCDR3 at least 65%, at least 70%, at least 75%, at least 80%, at least 85%, at least 90%, at least 95%, at least 99%, or 100% identical to SEQ ID NO: 1630, a HCDR1 at least 65%, at least 70%, at least 75%, at least 80%, at least 85%, at least 90%, at least 95%, at least 99%, or 100% identical to SEQ ID NO: 1632, a HCDR2 at least 65%, at least 70%, at least 75%, at least 80%, at least 85%, at least 90%, at least 95%, at least 99%, or 100% identical to SEQ ID NO: 1633, and a HCDR3 at least 65%, at least 70%, at least 75%, at least 80%, at least 85%, at least 90%, at least 95%, at least 99%, or 100% identical to SEQ ID NO: 1634;
a LCDR1 at least 65%, at least 70%, at least 75%, at least 80%, at least 85%, at least 90%, at least 95%, at least 99%, or 100% identical to SEQ ID NO: 1636, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1637, a HCDR1 at least 65%, at least 70%, at least 75%, at least 80%, at least 85%, at least 90%, at least 95%, at least 99%, or 100% identical to SEQ ID NO: 1639, a HCDR2 at least 65%, at least 70%, at least 75%, at least 80%, at least 85%, at least 90%, at least 95%, at least 99%, or 100% identical to SEQ ID NO: 1640, and a HCDR3 at least 65%, at least 70%, at least 75%, at least 80%, at least 85%, at least 90%, at least 95%, at least 99%, or 100% identical to SEQ ID NO: 1641;
a LCDR1 at least 65%, at least 70%, at least 75%, at least 80%, at least 85%, at least 90%, at least 95%, at least 99%, or 100% identical to SEQ ID NO: 1643, a LCDR2 at least 65%, at least 70%, at least 75%, at least 80%, at least 85%, at least 90%, at least 95%, at least 99%, or 100% identical to amino acid sequence SAS, a LCDR3 at least 65%, at least 70%, at least 75%, at least 80%, at least 85%, at least 90%, at least 95%, at least 99%, or 100% identical to SEQ ID NO: 1644, a HCDR1 at least 65%, at least 70%, at least 75%, at least 80%, at least 85%, at least 90%, at least 95%, at least 99%, or 100% identical to SEQ ID NO: 1646, a HCDR2 at least 65%, at least 70%, at least 75%, at least 80%, at least 85%, at least 90%, at least 95%, at least 99%, or 100% identical to SEQ ID NO: 1647, and a HCDR3 at least 65%, at least 70%, at least 75%, at least 80%, at least 85%, at least 90%, at least 95%, at least 99%, or 100% identical to SEQ ID NO: 1648;
a LCDR1 at least 65%, at least 70%, at least 75%, at least 80%, at least 85%, at least 90%, at least 95%, at least 99%, or 100% identical to SEQ ID NO: 1650, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1651, a HCDR1 at least 65%, at least 70%, at least 75%, at least 80%, at least 85%, at least 90%, at least 95%, at least 99%, or 100% identical to SEQ ID NO: 1653, a HCDR2 at least 65%, at least 70%, at least 75%, at least 80%, at least 85%, at least 90%, at least 95%, at least 99%, or 100% identical to SEQ ID NO: 1654, and a HCDR3 at least 65%, at least 70%, at least 75%, at least 80%, at least 85%, at least 90%, at least 95%, at least 99%, or 100% identical to SEQ ID NO: 1655;
a LCDR1 at least 65%, at least 70%, at least 75%, at least 80%, at least 85%, at least 90%, at least 95%, at least 99%, or 100% identical to SEQ ID NO: 1657, a LCDR2 at least 65%, at least 70%, at least 75%, at least 80%, at least 85%, at least 90%, at least 95%, at least 99%, or 100% identical to amino acid sequence DDS, a LCDR3 at least 65%, at least 70%, at least 75%, at least 80%, at least 85%, at least 90%, at least 95%, at least 99%, or 100% identical to SEQ ID NO: 1658, a HCDR1 at least 65%, at least 70%, at least 75%, at least 80%, at least 85%, at least 90%, at least 95%, at least 99%, or 100% identical to SEQ ID NO: 1660, a HCDR2 at least 65%, at least 70%, at least 75%, at least 80%, at least 85%, at least 90%, at least 95%, at least 99%, or 100% identical to SEQ ID NO: 1323, and a HCDR3 at least 65%, at least 70%, at least 75%, at least 80%, at least 85%, at least 90%, at least 95%, at least 99%, or 100% identical to SEQ ID NO: 1661;
a LCDR1 at least 65%, at least 70%, at least 75%, at least 80%, at least 85%, at least 90%, at least 95%, at least 99%, or 100% identical to SEQ ID NO: 1228, a LCDR2 at least 65%, at least 70%, at least 75%, at least 80%, at least 85%, at least 90%, at least 95%, at least 99%, or 100% identical to amino acid sequence FTS, a LCDR3 at least 65%, at least 70%, at least 75%, at least 80%, at least 85%, at least 90%, at least 95%, at least 99%, or 100% identical to SEQ ID NO: 1663, a HCDR1 at least 65%, at least 70%, at least 75%, at least 80%, at least 85%, at least 90%, at least 95%, at least 99%, or 100% identical to SEQ ID NO: 1665, a HCDR2 at least 65%, at least 70%, at least 75%, at least 80%, at least 85%, at least 90%, at least 95%, at least 99%, or 100% identical to SEQ ID NO: 1666, and a HCDR3 at least 65%, at least 70%, at least 75%, at least 80%, at least 85%, at least 90%, at least 95%, at least 99%, or 100% identical to SEQ ID NO: 1667;
a LCDR1 at least 65%, at least 70%, at least 75%, at least 80%, at least 85%, at least 90%, at least 95%, at least 99%, or 100% identical to SEQ ID NO: 1228, a LCDR2 at least 65%, at least 70%, at least 75%, at least 80%, at least 85%, at least 90%, at least 95%, at least 99%, or 100% identical to amino acid sequence FTS, a LCDR3 at least 65%, at least 70%, at least 75%, at least 80%, at least 85%, at least 90%, at least 95%, at least 99%, or 100% identical to SEQ ID NO: 1663, a HCDR1 at least 65%, at least 70%, at least 75%, at least 80%, at least 85%, at least 90%, at least 95%, at least 99%, or 100% identical to SEQ ID NO: 1203, a HCDR2 at least 65%, at least 70%, at least 75%, at least 80%, at least 85%, at least 90%, at least 95%, at least 99%, or 100% identical to SEQ ID NO: 1666, and a HCDR3 at least 65%, at least 70%, at least 75%, at least 80%, at least 85%, at least 90%, at least 95%, at least 99%, or 100% identical to SEQ ID NO: 1670;
a LCDR1 at least 65%, at least 70%, at least 75%, at least 80%, at least 85%, at least 90%, at least 95%, at least 99%, or 100% identical to SEQ ID NO: 1672, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1673, a HCDR1 at least 65%, at least 70%, at least 75%, at least 80%, at least 85%, at least 90%, at least 95%, at least 99%, or 100% identical to SEQ ID NO: 1675, a HCDR2 at least 65%, at least 70%, at least 75%, at least 80%, at least 85%, at least 90%, at least 95%, at least 99%, or 100% identical to SEQ ID NO: 1676, and a HCDR3 at least 65%, at least 70%, at least 75%, at least 80%, at least 85%, at least 90%, at least 95%, at least 99%, or 100% identical to SEQ ID NO: 1677;
a LCDR1 at least 65%, at least 70%, at least 75%, at least 80%, at least 85%, at least 90%, at least 95%, at least 99%, or 100% identical to SEQ ID NO: 1679, a LCDR2 at least 65%, at least 70%, at least 75%, at least 80%, at least 85%, at least 90%, at least 95%, at least 99%, or 100% identical to SEQ ID NO: 1680, a LCDR3 at least 65%, at least 70%, at least 75%, at least 80%, at least 85%, at least 90%, at least 95%, at least 99%, or 100% identical to SEQ ID NO: 1681, a HCDR1 at least 65%, at least 70%, at least 75%, at least 80%, at least 85%, at least 90%, at least 95%, at least 99%, or 100% identical to SEQ ID NO: 1683, a HCDR2 at least 65%, at least 70%, at least 75%, at least 80%, at least 85%, at least 90%, at least 95%, at least 99%, or 100% identical to SEQ ID NO: 1684, and a HCDR3 at least 65%, at least 70%, at least 75%, at least 80%, at least 85%, at least 90%, at least 95%, at least 99%, or 100% identical to SEQ ID NO: 1685;
a LCDR1 at least 65%, at least 70%, at least 75%, at least 80%, at least 85%, at least 90%, at least 95%, at least 99%, or 100% identical to SEQ ID NO: 1679, a LCDR2 at least 65%, at least 70%, at least 75%, at least 80%, at least 85%, at least 90%, at least 95%, at least 99%, or 100% identical to SEQ ID NO: 1680, a LCDR3 at least 65%, at least 70%, at least 75%, at least 80%, at least 85%, at least 90%, at least 95%, at least 99%, or 100% identical to SEQ ID NO: 1681, a HCDR1 at least 65%, at least 70%, at least 75%, at least 80%, at least 85%, at least 90%, at least 95%, at least 99%, or 100% identical to SEQ ID NO: 1295, a HCDR2 at least 65%, at least 70%, at least 75%, at least 80%, at least 85%, at least 90%, at least 95%, at least 99%, or 100% identical to SEQ ID NO: 1687, and a HCDR3 at least 65%, at least 70%, at least 75%, at least 80%, at least 85%, at least 90%, at least 95%, at least 99%, or 100% identical to SEQ ID NO: 1688;
a LCDR1 at least 65%, at least 70%, at least 75%, at least 80%, at least 85%, at least 90%, at least 95%, at least 99%, or 100% identical to SEQ ID NO: 1689, a LCDR2 at least 65%, at least 70%, at least 75%, at least 80%, at least 85%, at least 90%, at least 95%, at least 99%, or 100% identical to SEQ ID NO: 1690, a LCDR3 at least 65%, at least 70%, at least 75%, at least 80%, at least 85%, at least 90%, at least 95%, at least 99%, or 100% identical to SEQ ID NO: 1691, a HCDR1 at least 65%, at least 70%, at least 75%, at least 80%, at least 85%, at least 90%, at least 95%, at least 99%, or 100% identical to SEQ ID NO: 1693, a HCDR2 at least 65%, at least 70%, at least 75%, at least 80%, at least 85%, at least 90%, at least 95%, at least 99%, or 100% identical to SEQ ID NO: 1694, and a HCDR3 at least 65%, at least 70%, at least 75%, at least 80%, at least 85%, at least 90%, at least 95%, at least 99%, or 100% identical to SEQ ID NO: 1695;
a LCDR1 at least 65%, at least 70%, at least 75%, at least 80%, at least 85%, at least 90%, at least 95%, at least 99%, or 100% identical to SEQ ID NO: 1697, a LCDR2 at least 65%, at least 70%, at least 75%, at least 80%, at least 85%, at least 90%, at least 95%, at least 99%, or 100% identical to SEQ ID NO: 1698, a LCDR3 at least 65%, at least 70%, at least 75%, at least 80%, at least 85%, at least 90%, at least 95%, at least 99%, or 100% identical to SEQ ID NO: 1699, a HCDR1 at least 65%, at least 70%, at least 75%, at least 80%, at least 85%, at least 90%, at least 95%, at least 99%, or 100% identical to SEQ ID NO: 1701, a HCDR2 at least 65%, at least 70%, at least 75%, at least 80%, at least 85%, at least 90%, at least 95%, at least 99%, or 100% identical to SEQ ID NO: 1702, and a HCDR3 at least 65%, at least 70%, at least 75%, at least 80%, at least 85%, at least 90%, at least 95%, at least 99%, or 100% identical to SEQ ID NO: 1703;
a LCDR1 at least 65%, at least 70%, at least 75%, at least 80%, at least 85%, at least 90%, at least 95%, at least 99%, or 100% identical to SEQ ID NO: 1538, a LCDR2 at least 65%, at least 70%, at least 75%, at least 80%, at least 85%, at least 90%, at least 95%, at least 99%, or 100% identical to amino acid sequence YTS, a LCDR3 at least 65%, at least 70%, at least 75%, at least 80%, at least 85%, at least 90%, at least 95%, at least 99%, or 100% identical to SEQ ID NO: 1691, a HCDR1 at least 65%, at least 70%, at least 75%, at least 80%, at least 85%, at least 90%, at least 95%, at least 99%, or 100% identical to SEQ ID NO: 1705, a HCDR2 at least 65%, at least 70%, at least 75%, at least 80%, at least 85%, at least 90%, at least 95%, at least 99%, or 100% identical to SEQ ID NO: 1706, and a HCDR3 at least 65%, at least 70%, at least 75%, at least 80%, at least 85%, at least 90%, at least 95%, at least 99%, or 100% identical to SEQ ID NO: 1707;
a LCDR1 at least 65%, at least 70%, at least 75%, at least 80%, at least 85%, at least 90%, at least 95%, at least 99%, or 100% identical to SEQ ID NO: 1708, a LCDR2 at least 65%, at least 70%, at least 75%, at least 80%, at least 85%, at least 90%, at least 95%, at least 99%, or 100% identical to SEQ ID NO: 1709, a LCDR3 at least 65%, at least 70%, at least 75%, at least 80%, at least 85%, at least 90%, at least 95%, at least 99%, or 100% identical to SEQ ID NO: 1710, a HCDR1 at least 65%, at least 70%, at least 75%, at least 80%, at least 85%, at least 90%, at least 95%, at least 99%, or 100% identical to SEQ ID NO: 1683, a HCDR2 at least 65%, at least 70%, at least 75%, at least 80%, at least 85%, at least 90%, at least 95%, at least 99%, or 100% identical to SEQ ID NO: 1712, and a HCDR3 at least 65%, at least 70%, at least 75%, at least 80%, at least 85%, at least 90%, at least 95%, at least 99%, or 100% identical to SEQ ID NO: 1713;
a LCDR1 at least 65%, at least 70%, at least 75%, at least 80%, at least 85%, at least 90%, at least 95%, at least 99%, or 100% identical to SEQ ID NO: 1715, a LCDR2 at least 65%, at least 70%, at least 75%, at least 80%, at least 85%, at least 90%, at least 95%, at least 99%, or 100% identical to SEQ ID NO: 1709, a LCDR3 at least 65%, at least 70%, at least 75%, at least 80%, at least 85%, at least 90%, at least 95%, at least 99%, or 100% identical to SEQ ID NO: 1710, a HCDR1 at least 65%, at least 70%, at least 75%, at least 80%, at least 85%, at least 90%, at least 95%, at least 99%, or 100% identical to SEQ ID NO: 1716, a HCDR2 at least 65%, at least 70%, at least 75%, at least 80%, at least 85%, at least 90%, at least 95%, at least 99%, or 100% identical to SEQ ID NO: 1717, and a HCDR3 at least 65%, at least 70%, at least 75%, at least 80%, at least 85%, at least 90%, at least 95%, at least 99%, or 100% identical to SEQ ID NO: 1713;
a LCDR1 at least 65%, at least 70%, at least 75%, at least 80%, at least 85%, at least 90%, at least 95%, at least 99%, or 100% identical to SEQ ID NO: 1718, a LCDR2 at least 65%, at least 70%, at least 75%, at least 80%, at least 85%, at least 90%, at least 95%, at least 99%, or 100% identical to SEQ ID NO: 1709, a LCDR3 at least 65%, at least 70%, at least 75%, at least 80%, at least 85%, at least 90%, at least 95%, at least 99%, or 100% identical to SEQ ID NO: 1710, a HCDR1 at least 65%, at least 70%, at least 75%, at least 80%, at least 85%, at least 90%, at least 95%, at least 99%, or 100% identical to SEQ ID NO: 1719, a HCDR2 at least 65%, at least 70%, at least 75%, at least 80%, at least 85%, at least 90%, at least 95%, at least 99%, or 100% identical to SEQ ID NO: 1720, and a HCDR3 at least 65%, at least 70%, at least 75%, at least 80%, at least 85%, at least 90%, at least 95%, at least 99%, or 100% identical to SEQ ID NO: 1713;
a LCDR1 at least 65%, at least 70%, at least 75%, at least 80%, at least 85%, at least 90%, at least 95%, at least 99%, or 100% identical to SEQ ID NO: 1718, a LCDR2 at least 65%, at least 70%, at least 75%, at least 80%, at least 85%, at least 90%, at least 95%, at least 99%, or 100% identical to SEQ ID NO: 1709, a LCDR3 at least 65%, at least 70%, at least 75%, at least 80%, at least 85%, at least 90%, at least 95%, at least 99%, or 100% identical to SEQ ID NO: 1710, a HCDR1 at least 65%, at least 70%, at least 75%, at least 80%, at least 85%, at least 90%, at least 95%, at least 99%, or 100% identical to SEQ ID NO: 1719, a HCDR2 at least 65%, at least 70%, at least 75%, at least 80%, at least 85%, at least 90%, at least 95%, at least 99%, or 100% identical to SEQ ID NO: 1720, and a HCDR3 at least 65%, at least 70%, at least 75%, at least 80%, at least 85%, at least 90%, at least 95%, at least 99%, or 100% identical to SEQ ID NO: 1713;
a LCDR1 at least 65%, at least 70%, at least 75%, at least 80%, at least 85%, at least 90%, at least 95%, at least 99%, or 100% identical to SEQ ID NO: 1723, a LCDR2 at least 65%, at least 70%, at least 75%, at least 80%, at least 85%, at least 90%, at least 95%, at least 99%, or 100% identical to SEQ ID NO: 1709, a LCDR3 at least 65%, at least 70%, at least 75%, at least 80%, at least 85%, at least 90%, at least 95%, at least 99%, or 100% identical to SEQ ID NO: 1710, a HCDR1 at least 65%, at least 70%, at least 75%, at least 80%, at least 85%, at least 90%, at least 95%, at least 99%, or 100% identical to SEQ ID NO: 1716, a HCDR2 at least 65%, at least 70%, at least 75%, at least 80%, at least 85%, at least 90%, at least 95%, at least 99%, or 100% identical to SEQ ID NO: 1725, and a HCDR3 at least 65%, at least 70%, at least 75%, at least 80%, at least 85%, at least 90%, at least 95%, at least 99%, or 100% identical to SEQ ID NO: 1713;
a LCDR1 at least 65%, at least 70%, at least 75%, at least 80%, at least 85%, at least 90%, at least 95%, at least 99%, or 100% identical to SEQ ID NO: 1727, a LCDR2 at least 65%, at least 70%, at least 75%, at least 80%, at least 85%, at least 90%, at least 95%, at least 99%, or 100% identical to SEQ ID NO: 1728, a LCDR3 at least 65%, at least 70%, at least 75%, at least 80%, at least 85%, at least 90%, at least 95%, at least 99%, or 100% identical to SEQ ID NO: 1729, a HCDR1 at least 65%, at least 70%, at least 75%, at least 80%, at least 85%, at least 90%, at least 95%, at least 99%, or 100% identical to SEQ ID NO: 1731, a HCDR2 at least 65%, at least 70%, at least 75%, at least 80%, at least 85%, at least 90%, at least 95%, at least 99%, or 100% identical to SEQ ID NO: 1732, and a HCDR3 at least 65%, at least 70%, at least 75%, at least 80%, at least 85%, at least 90%, at least 95%, at least 99%, or 100% identical to SEQ ID NO: 1733;
a LCDR1 at least 65%, at least 70%, at least 75%, at least 80%, at least 85%, at least 90%, at least 95%, at least 99%, or 100% identical to SEQ ID NO: 1727, a LCDR2 at least 65%, at least 70%, at least 75%, at least 80%, at least 85%, at least 90%, at least 95%, at least 99%, or 100% identical to SEQ ID NO: 1728, a LCDR3 at least 65%, at least 70%, at least 75%, at least 80%, at least 85%, at least 90%, at least 95%, at least 99%, or 100% identical to SEQ ID NO: 1735, a HCDR1 at least 65%, at least 70%, at least 75%, at least 80%, at least 85%, at least 90%, at least 95%, at least 99%, or 100% identical to SEQ ID NO: 1736, a HCDR2 at least 65%, at least 70%, at least 75%, at least 80%, at least 85%, at least 90%, at least 95%, at least 99%, or 100% identical to SEQ ID NO: 1737, and a HCDR3 at least 65%, at least 70%, at least 75%, at least 80%, at least 85%, at least 90%, at least 95%, at least 99%, or 100% identical to SEQ ID NO: 1733;
a LCDR1 at least 65%, at least 70%, at least 75%, at least 80%, at least 85%, at least 90%, at least 95%, at least 99%, or 100% identical to SEQ ID NO: 1738, a LCDR2 at least 65%, at least 70%, at least 75%, at least 80%, at least 85%, at least 90%, at least 95%, at least 99%, or 100% identical to SEQ ID NO: 1739, a LCDR3 at least 65%, at least 70%, at least 75%, at least 80%, at least 85%, at least 90%, at least 95%, at least 99%, or 100% identical to SEQ ID NO: 1729, a HCDR1 at least 65%, at least 70%, at least 75%, at least 80%, at least 85%, at least 90%, at least 95%, at least 99%, or 100% identical to SEQ ID NO: 1736, a HCDR2 at least 65%, at least 70%, at least 75%, at least 80%, at least 85%, at least 90%, at least 95%, at least 99%, or 100% identical to SEQ ID NO: 1741, and a HCDR3 at least 65%, at least 70%, at least 75%, at least 80%, at least 85%, at least 90%, at least 95%, at least 99%, or 100% identical to SEQ ID NO: 1733;
a LCDR1 at least 65%, at least 70%, at least 75%, at least 80%, at least 85%, at least 90%, at least 95%, at least 99%, or 100% identical to SEQ ID NO: 1743, a LCDR2 at least 65%, at least 70%, at least 75%, at least 80%, at least 85%, at least 90%, at least 95%, at least 99%, or 100% identical to SEQ ID NO: 1744, a LCDR3 at least 65%, at least 70%, at least 75%, at least 80%, at least 85%, at least 90%, at least 95%, at least 99%, or 100% identical to SEQ ID NO: 1745, a HCDR1 at least 65%, at least 70%, at least 75%, at least 80%, at least 85%, at least 90%, at least 95%, at least 99%, or 100% identical to SEQ ID NO: 1747, a HCDR2 at least 65%, at least 70%, at least 75%, at least 80%, at least 85%, at least 90%, at least 95%, at least 99%, or 100% identical to SEQ ID NO: 1748, and a HCDR3 at least 65%, at least 70%, at least 75%, at least 80%, at least 85%, at least 90%, at least 95%, at least 99%, or 100% identical to SEQ ID NO: 1749;
a LCDR1 at least 65%, at least 70%, at least 75%, at least 80%, at least 85%, at least 90%, at least 95%, at least 99%, or 100% identical to SEQ ID NO: 1743, a LCDR2 at least 65%, at least 70%, at least 75%, at least 80%, at least 85%, at least 90%, at least 95%, at least 99%, or 100% identical to SEQ ID NO: 1744, a LCDR3 at least 65%, at least 70%, at least 75%, at least 80%, at least 85%, at least 90%, at least 95%, at least 99%, or 100% identical to SEQ ID NO: 1751, a HCDR1 at least 65%, at least 70%, at least 75%, at least 80%, at least 85%, at least 90%, at least 95%, at least 99%, or 100% identical to SEQ ID NO: 1747, a HCDR2 at least 65%, at least 70%, at least 75%, at least 80%, at least 85%, at least 90%, at least 95%, at least 99%, or 100% identical to SEQ ID NO: 1748, and a HCDR3 at least 65%, at least 70%, at least 75%, at least 80%, at least 85%, at least 90%, at least 95%, at least 99%, or 100% identical to SEQ ID NO: 1749;
a LCDR1 at least 65%, at least 70%, at least 75%, at least 80%, at least 85%, at least 90%, at least 95%, at least 99%, or 100% identical to SEQ ID NO: 1743, a LCDR2 at least 65%, at least 70%, at least 75%, at least 80%, at least 85%, at least 90%, at least 95%, at least 99%, or 100% identical to SEQ ID NO: 1744, a LCDR3 at least 65%, at least 70%, at least 75%, at least 80%, at least 85%, at least 90%, at least 95%, at least 99%, or 100% identical to SEQ ID NO: 1751, a HCDR1 at least 65%, at least 70%, at least 75%, at least 80%, at least 85%, at least 90%, at least 95%, at least 99%, or 100% identical to SEQ ID NO: 1747, a HCDR2 at least 65%, at least 70%, at least 75%, at least 80%, at least 85%, at least 90%, at least 95%, at least 99%, or 100% identical to SEQ ID NO: 1748, and a HCDR3 at least 65%, at least 70%, at least 75%, at least 80%, at least 85%, at least 90%, at least 95%, at least 99%, or 100% identical to SEQ ID NO: 1749;
a LCDR1 at least 65%, at least 70%, at least 75%, at least 80%, at least 85%, at least 90%, at least 95%, at least 99%, or 100% identical to SEQ ID NO: 1752, a LCDR2 at least 65%, at least 70%, at least 75%, at least 80%, at least 85%, at least 90%, at least 95%, at least 99%, or 100% identical to SEQ ID NO: 1753, a LCDR3 at least 65%, at least 70%, at least 75%, at least 80%, at least 85%, at least 90%, at least 95%, at least 99%, or 100% identical to SEQ ID NO: 1754, a HCDR1 at least 65%, at least 70%, at least 75%, at least 80%, at least 85%, at least 90%, at least 95%, at least 99%, or 100% identical to SEQ ID NO: 1756, a HCDR2 at least 65%, at least 70%, at least 75%, at least 80%, at least 85%, at least 90%, at least 95%, at least 99%, or 100% identical to SEQ ID NO: 1757, and a HCDR3 at least 65%, at least 70%, at least 75%, at least 80%, at least 85%, at least 90%, at least 95%, at least 99%, or 100% identical to SEQ ID NO: 1758;
a LCDR1 at least 65%, at least 70%, at least 75%, at least 80%, at least 85%, at least 90%, at least 95%, at least 99%, or 100% identical to SEQ ID NO: 1752, a LCDR2 at least 65%, at least 70%, at least 75%, at least 80%, at least 85%, at least 90%, at least 95%, at least 99%, or 100% identical to SEQ ID NO: 1753, a LCDR3 at least 65%, at least 70%, at least 75%, at least 80%, at least 85%, at least 90%, at least 95%, at least 99%, or 100% identical to SEQ ID NO: 1754, a HCDR1 at least 65%, at least 70%, at least 75%, at least 80%, at least 85%, at least 90%, at least 95%, at least 99%, or 100% identical to SEQ ID NO: 1756, a HCDR2 at least 65%, at least 70%, at least 75%, at least 80%, at least 85%, at least 90%, at least 95%, at least 99%, or 100% identical to SEQ ID NO: 1757, and a HCDR3 at least 65%, at least 70%, at least 75%, at least 80%, at least 85%, at least 90%, at least 95%, at least 99%, or 100% identical to SEQ ID NO: 1758;
a LCDR1 at least 65%, at least 70%, at least 75%, at least 80%, at least 85%, at least 90%, at least 95%, at least 99%, or 100% identical to SEQ ID NO: 1761, a LCDR2 at least 65%, at least 70%, at least 75%, at least 80%, at least 85%, at least 90%, at least 95%, at least 99%, or 100% identical to amino acid sequence DT, a LCDR3 at least 65%, at least 70%, at least 75%, at least 80%, at least 85%, at least 90%, at least 95%, at least 99%, or 100% identical to SEQ ID NO: 1729, a HCDR1 at least 65%, at least 70%, at least 75%, at least 80%, at least 85%, at least 90%, at least 95%, at least 99%, or 100% identical to SEQ ID NO: 1243, a HCDR2 at least 65%, at least 70%, at least 75%, at least 80%, at least 85%, at least 90%, at least 95%, at least 99%, or 100% identical to SEQ ID NO: 1244, and a HCDR3 at least 65%, at least 70%, at least 75%, at least 80%, at least 85%, at least 90%, at least 95%, at least 99%, or 100% identical to SEQ ID NO: 1762;
a LCDR1 at least 65%, at least 70%, at least 75%, at least 80%, at least 85%, at least 90%, at least 95%, at least 99%, or 100% identical to SEQ ID NO: 1763, a LCDR2 at least 65%, at least 70%, at least 75%, at least 80%, at least 85%, at least 90%, at least 95%, at least 99%, or 100% identical to amino acid sequence AT, a LCDR3 at least 65%, at least 70%, at least 75%, at least 80%, at least 85%, at least 90%, at least 95%, at least 99%, or 100% identical to SEQ ID NO: 1195, a HCDR1 at least 65%, at least 70%, at least 75%, at least 80%, at least 85%, at least 90%, at least 95%, at least 99%, or 100% identical to SEQ ID NO: 1184, a HCDR2 at least 65%, at least 70%, at least 75%, at least 80%, at least 85%, at least 90%, at least 95%, at least 99%, or 100% identical to SEQ ID NO: 1185, and a HCDR3 at least 65%, at least 70%, at least 75%, at least 80%, at least 85%, at least 90%, at least 95%, at least 99%, or 100% identical to SEQ ID NO: 1197;
a LCDR1 at least 65%, at least 70%, at least 75%, at least 80%, at least 85%, at least 90%, at least 95%, at least 99%, or 100% identical to SEQ ID NO: 1763, a LCDR2 at least 65%, at least 70%, at least 75%, at least 80%, at least 85%, at least 90%, at least 95%, at least 99%, or 100% identical to amino acid sequence AT, a LCDR3 at least 65%, at least 70%, at least 75%, at least 80%, at least 85%, at least 90%, at least 95%, at least 99%, or 100% identical to SEQ ID NO: 1195, a HCDR1 at least 65%, at least 70%, at least 75%, at least 80%, at least 85%, at least 90%, at least 95%, at least 99%, or 100% identical to SEQ ID NO: 1184, a HCDR2 at least 65%, at least 70%, at least 75%, at least 80%, at least 85%, at least 90%, at least 95%, at least 99%, or 100% identical to SEQ ID NO: 1767, and a HCDR3 at least 65%, at least 70%, at least 75%, at least 80%, at least 85%, at least 90%, at least 95%, at least 99%, or 100% identical to SEQ ID NO: 1197;
a LCDR1 at least 65%, at least 70%, at least 75%, at least 80%, at least 85%, at least 90%, at least 95%, at least 99%, or 100% identical to SEQ ID NO: 1769, a LCDR2 at least 65%, at least 70%, at least 75%, at least 80%, at least 85%, at least 90%, at least 95%, at least 99%, or 100% identical to SEQ ID NO: 1770, a LCDR3 at least 65%, at least 70%, at least 75%, at least 80%, at least 85%, at least 90%, at least 95%, at least 99%, or 100% identical to SEQ ID NO: 1182, a HCDR1 at least 65%, at least 70%, at least 75%, at least 80%, at least 85%, at least 90%, at least 95%, at least 99%, or 100% identical to SEQ ID NO: 1772, a HCDR2 at least 65%, at least 70%, at least 75%, at least 80%, at least 85%, at least 90%, at least 95%, at least 99%, or 100% identical to SEQ ID NO: 1773, and a HCDR3 at least 65%, at least 70%, at least 75%, at least 80%, at least 85%, at least 90%, at least 95%, at least 99%, or 100% identical to SEQ ID NO: 1774;
a LCDR1 at least 65%, at least 70%, at least 75%, at least 80%, at least 85%, at least 90%, at least 95%, at least 99%, or 100% identical to SEQ ID NO: 1776, a LCDR2 at least 65%, at least 70%, at least 75%, at least 80%, at least 85%, at least 90%, at least 95%, at least 99%, or 100% identical to SEQ ID NO: 1777, a LCDR3 at least 65%, at least 70%, at least 75%, at least 80%, at least 85%, at least 90%, at least 95%, at least 99%, or 100% identical to SEQ ID NO: 1778, a HCDR1 at least 65%, at least 70%, at least 75%, at least 80%, at least 85%, at least 90%, at least 95%, at least 99%, or 100% identical to SEQ ID NO: 1780, a HCDR2 at least 65%, at least 70%, at least 75%, at least 80%, at least 85%, at least 90%, at least 95%, at least 99%, or 100% identical to SEQ ID NO: 1781, and a HCDR3 at least 65%, at least 70%, at least 75%, at least 80%, at least 85%, at least 90%, at least 95%, at least 99%, or 100% identical to SEQ ID NO: 1782;
a LCDR1 at least 65%, at least 70%, at least 75%, at least 80%, at least 85%, at least 90%, at least 95%, at least 99%, or 100% identical to SEQ ID NO: 1776, a LCDR2 at least 65%, at least 70%, at least 75%, at least 80%, at least 85%, at least 90%, at least 95%, at least 99%, or 100% identical to SEQ ID NO: 1777, a LCDR3 at least 65%, at least 70%, at least 75%, at least 80%, at least 85%, at least 90%, at least 95%, at least 99%, or 100% identical to SEQ ID NO: 1778, a HCDR1 at least 65%, at least 70%, at least 75%, at least 80%, at least 85%, at least 90%, at least 95%, at least 99%, or 100% identical to SEQ ID NO: 1780, a HCDR2 at least 65%, at least 70%, at least 75%, at least 80%, at least 85%, at least 90%, at least 95%, at least 99%, or 100% identical to SEQ ID NO: 1781, and a HCDR3 at least 65%, at least 70%, at least 75%, at least 80%, at least 85%, at least 90%, at least 95%, at least 99%, or 100% identical to SEQ ID NO: 1782;
a LCDR1 at least 65%, at least 70%, at least 75%, at least 80%, at least 85%, at least 90%, at least 95%, at least 99%, or 100% identical to SEQ ID NO: 1786, a LCDR2 at least 65%, at least 70%, at least 75%, at least 80%, at least 85%, at least 90%, at least 95%, at least 99%, or 100% identical to SEQ ID NO: 1777, a LCDR3 at least 65%, at least 70%, at least 75%, at least 80%, at least 85%, at least 90%, at least 95%, at least 99%, or 100% identical to SEQ ID NO: 1787, a HCDR1 at least 65%, at least 70%, at least 75%, at least 80%, at least 85%, at least 90%, at least 95%, at least 99%, or 100% identical to SEQ ID NO: 1789, a HCDR2 at least 65%, at least 70%, at least 75%, at least 80%, at least 85%, at least 90%, at least 95%, at least 99%, or 100% identical to SEQ ID NO: 1790, and a HCDR3 at least 65%, at least 70%, at least 75%, at least 80%, at least 85%, at least 90%, at least 95%, at least 99%, or 100% identical to SEQ ID NO: 1791;
a LCDR1 at least 65%, at least 70%, at least 75%, at least 80%, at least 85%, at least 90%, at least 95%, at least 99%, or 100% identical to SEQ ID NO: 1793, a LCDR2 at least 65%, at least 70%, at least 75%, at least 80%, at least 85%, at least 90%, at least 95%, at least 99%, or 100% identical to amino acid sequence YTS, a LCDR3 at least 65%, at least 70%, at least 75%, at least 80%, at least 85%, at least 90%, at least 95%, at least 99%, or 100% identical to SEQ ID NO: 1229, a HCDR1 at least 65%, at least 70%, at least 75%, at least 80%, at least 85%, at least 90%, at least 95%, at least 99%, or 100% identical to SEQ ID NO: 1795, a HCDR2 at least 65%, at least 70%, at least 75%, at least 80%, at least 85%, at least 90%, at least 95%, at least 99%, or 100% identical to SEQ ID NO: 1159, and a HCDR3 at least 65%, at least 70%, at least 75%, at least 80%, at least 85%, at least 90%, at least 95%, at least 99%, or 100% identical to SEQ ID NO: 1796;
a LCDR1 at least 65%, at least 70%, at least 75%, at least 80%, at least 85%, at least 90%, at least 95%, at least 99%, or 100% identical to SEQ ID NO: 1798, a LCDR2 at least 65%, at least 70%, at least 75%, at least 80%, at least 85%, at least 90%, at least 95%, at least 99%, or 100% identical to amino acid sequence DTS, a LCDR3 at least 65%, at least 70%, at least 75%, at least 80%, at least 85%, at least 90%, at least 95%, at least 99%, or 100% identical to SEQ ID NO: 1799, a HCDR1 at least 65%, at least 70%, at least 75%, at least 80%, at least 85%, at least 90%, at least 95%, at least 99%, or 100% identical to SEQ ID NO: 1801, a HCDR2 at least 65%, at least 70%, at least 75%, at least 80%, at least 85%, at least 90%, at least 95%, at least 99%, or 100% identical to SEQ ID NO: 1802, and a HCDR3 at least 65%, at least 70%, at least 75%, at least 80%, at least 85%, at least 90%, at least 95%, at least 99%, or 100% identical to SEQ ID NO: 1803;
a LCDR1 at least 65%, at least 70%, at least 75%, at least 80%, at least 85%, at least 90%, at least 95%, at least 99%, or 100% identical to SEQ ID NO: 1428, a LCDR2 at least 65%, at least 70%, at least 75%, at least 80%, at least 85%, at least 90%, at least 95%, at least 99%, or 100% identical to amino acid sequence GAS, a LCDR3 at least 65%, at least 70%, at least 75%, at least 80%, at least 85%, at least 90%, at least 95%, at least 99%, or 100% identical to SEQ ID NO: 1805, a HCDR1 at least 65%, at least 70%, at least 75%, at least 80%, at least 85%, at least 90%, at least 95%, at least 99%, or 100% identical to SEQ ID NO: 1577, a HCDR2 at least 65%, at least 70%, at least 75%, at least 80%, at least 85%, at least 90%, at least 95%, at least 99%, or 100% identical to SEQ ID NO: 1807, and a HCDR3 at least 65%, at least 70%, at least 75%, at least 80%, at least 85%, at least 90%, at least 95%, at least 99%, or 100% identical to SEQ ID NO: 1808;
a LCDR1 at least 65%, at least 70%, at least 75%, at least 80%, at least 85%, at least 90%, at least 95%, at least 99%, or 100% identical to SEQ ID NO: 1810, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1811, a HCDR1 at least 65%, at least 70%, at least 75%, at least 80%, at least 85%, at least 90%, at least 95%, at least 99%, or 100% identical to SEQ ID NO: 1813, a HCDR2 at least 65%, at least 70%, at least 75%, at least 80%, at least 85%, at least 90%, at least 95%, at least 99%, or 100% identical to SEQ ID NO: 1814, and a HCDR3 at least 65%, at least 70%, at least 75%, at least 80%, at least 85%, at least 90%, at least 95%, at least 99%, or 100% identical to SEQ ID NO: 1815;
a LCDR1 at least 65%, at least 70%, at least 75%, at least 80%, at least 85%, at least 90%, at least 95%, at least 99%, or 100% identical to SEQ ID NO: 1817, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1818, a HCDR1 at least 65%, at least 70%, at least 75%, at least 80%, at least 85%, at least 90%, at least 95%, at least 99%, or 100% identical to SEQ ID NO: 1820, a HCDR2 at least 65%, at least 70%, at least 75%, at least 80%, at least 85%, at least 90%, at least 95%, at least 99%, or 100% identical to SEQ ID NO: 1821, and a HCDR3 at least 65%, at least 70%, at least 75%, at least 80%, at least 85%, at least 90%, at least 95%, at least 99%, or 100% identical to SEQ ID NO: 1822;
a LCDR1 at least 65%, at least 70%, at least 75%, at least 80%, at least 85%, at least 90%, at least 95%, at least 99%, or 100% identical to SEQ ID NO: 1824, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1825, a HCDR1 at least 65%, at least 70%, at least 75%, at least 80%, at least 85%, at least 90%, at least 95%, at least 99%, or 100% identical to SEQ ID NO: 1827, a HCDR2 at least 65%, at least 70%, at least 75%, at least 80%, at least 85%, at least 90%, at least 95%, at least 99%, or 100% identical to SEQ ID NO: 1828, and a HCDR3 at least 65%, at least 70%, at least 75%, at least 80%, at least 85%, at least 90%, at least 95%, at least 99%, or 100% identical to SEQ ID NO: 1829;
a LCDR1 at least 65%, at least 70%, at least 75%, at least 80%, at least 85%, at least 90%, at least 95%, at least 99%, or 100% identical to SEQ ID NO: 1977, a LCDR2 at least 65%, at least 70%, at least 75%, at least 80%, at least 85%, at least 90%, at least 95%, at least 99%, or 100% identical to SEQ ID NO: 1978, a LCDR3 at least 65%, at least 70%, at least 75%, at least 80%, at least 85%, at least 90%, at least 95%, at least 99%, or 100% identical to SEQ ID NO: 1979, a HCDR1 at least 65%, at least 70%, at least 75%, at least 80%, at least 85%, at least 90%, at least 95%, at least 99%, or 100% identical to SEQ ID NO: 1981, a HCDR2 at least 65%, at least 70%, at least 75%, at least 80%, at least 85%, at least 90%, at least 95%, at least 99%, or 100% identical to SEQ ID NO: 1982, and a HCDR3 at least 65%, at least 70%, at least 75%, at least 80%, at least 85%, at least 90%, at least 95%, at least 99%, or 100% identical to SEQ ID NO: 1983;
a LCDR1 at least 65%, at least 70%, at least 75%, at least 80%, at least 85%, at least 90%, at least 95%, at least 99%, or 100% identical to SEQ ID NO: 1985, a LCDR2 at least 65%, at least 70%, at least 75%, at least 80%, at least 85%, at least 90%, at least 95%, at least 99%, or 100% identical to SEQ ID NO: 1986, a LCDR3 at least 65%, at least 70%, at least 75%, at least 80%, at least 85%, at least 90%, at least 95%, at least 99%, or 100% identical to SEQ ID NO: 1987, a HCDR1 at least 65%, at least 70%, at least 75%, at least 80%, at least 85%, at least 90%, at least 95%, at least 99%, or 100% identical to SEQ ID NO: 1989, a HCDR2 at least 65%, at least 70%, at least 75%, at least 80%, at least 85%, at least 90%, at least 95%, at least 99%, or 100% identical to SEQ ID NO: 1990, and a HCDR3 at least 65%, at least 70%, at least 75%, at least 80%, at least 85%, at least 90%, at least 95%, at least 99%, or 100% identical to SEQ ID NO: 1991;
a LCDR1 at least 65%, at least 70%, at least 75%, at least 80%, at least 85%, at least 90%, at least 95%, at least 99%, or 100% identical to SEQ ID NO: 1993, a LCDR2 at least 65%, at least 70%, at least 75%, at least 80%, at least 85%, at least 90%, at least 95%, at least 99%, or 100% identical to SEQ ID NO: 1994, a LCDR3 at least 65%, at least 70%, at least 75%, at least 80%, at least 85%, at least 90%, at least 95%, at least 99%, or 100% identical to SEQ ID NO: 1995, a HCDR1 at least 65%, at least 70%, at least 75%, at least 80%, at least 85%, at least 90%, at least 95%, at least 99%, or 100% identical to SEQ ID NO: 1997, a HCDR2 at least 65%, at least 70%, at least 75%, at least 80%, at least 85%, at least 90%, at least 95%, at least 99%, or 100% identical to SEQ ID NO: 1998, and a HCDR3 at least 65%, at least 70%, at least 75%, at least 80%, at least 85%, at least 90%, at least 95%, at least 99%, or 100% identical to SEQ ID NO: 1999;
a LCDR1 at least 65%, at least 70%, at least 75%, at least 80%, at least 85%, at least 90%, at least 95%, at least 99%, or 100% identical to SEQ ID NO: 2001, a LCDR2 at least 65%, at least 70%, at least 75%, at least 80%, at least 85%, at least 90%, at least 95%, at least 99%, or 100% identical to SEQ ID NO: 2002, a LCDR3 at least 65%, at least 70%, at least 75%, at least 80%, at least 85%, at least 90%, at least 95%, at least 99%, or 100% identical to SEQ ID NO: 2003, a HCDR1 at least 65%, at least 70%, at least 75%, at least 80%, at least 85%, at least 90%, at least 95%, at least 99%, or 100% identical to SEQ ID NO: 2005, a HCDR2 at least 65%, at least 70%, at least 75%, at least 80%, at least 85%, at least 90%, at least 95%, at least 99%, or 100% identical to SEQ ID NO: 2006, and a HCDR3 at least 65%, at least 70%, at least 75%, at least 80%, at least 85%, at least 90%, at least 95%, at least 99%, or 100% identical to SEQ ID NO: 2007;
a LCDR1 at least 65%, at least 70%, at least 75%, at least 80%, at least 85%, at least 90%, at least 95%, at least 99%, or 100% identical to SEQ ID NO: 1831, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1832, a HCDR1 at least 65%, at least 70%, at least 75%, at least 80%, at least 85%, at least 90%, at least 95%, at least 99%, or 100% identical to SEQ ID NO: 1834, a HCDR2 at least 65%, at least 70%, at least 75%, at least 80%, at least 85%, at least 90%, at least 95%, at least 99%, or 100% identical to SEQ ID NO: 1835, and a HCDR3 at least 65%, at least 70%, at least 75%, at least 80%, at least 85%, at least 90%, at least 95%, at least 99%, or 100% identical to SEQ ID NO: 1836;
a LCDR1 at least 65%, at least 70%, at least 75%, at least 80%, at least 85%, at least 90%, at least 95%, at least 99%, or 100% identical to SEQ ID NO: 1838, a LCDR2 at least 65%, at least 70%, at least 75%, at least 80%, at least 85%, at least 90%, at least 95%, at least 99%, or 100% identical to amino acid sequence WAS, a LCDR3 at least 65%, at least 70%, at least 75%, at least 80%, at least 85%, at least 90%, at least 95%, at least 99%, or 100% identical to SEQ ID NO: 1839, a HCDR1 at least 65%, at least 70%, at least 75%, at least 80%, at least 85%, at least 90%, at least 95%, at least 99%, or 100% identical to SEQ ID NO: 1841, a HCDR2 at least 65%, at least 70%, at least 75%, at least 80%, at least 85%, at least 90%, at least 95%, at least 99%, or 100% identical to SEQ ID NO: 1842, and a HCDR3 at least 65%, at least 70%, at least 75%, at least 80%, at least 85%, at least 90%, at least 95%, at least 99%, or 100% identical to SEQ ID NO: 1843;
a LCDR1 at least 65%, at least 70%, at least 75%, at least 80%, at least 85%, at least 90%, at least 95%, at least 99%, or 100% identical to SEQ ID NO: 1845, a LCDR2 at least 65%, at least 70%, at least 75%, at least 80%, at least 85%, at least 90%, at least 95%, at least 99%, or 100% identical to amino acid sequence EVS, a LCDR3 at least 65%, at least 70%, at least 75%, at least 80%, at least 85%, at least 90%, at least 95%, at least 99%, or 100% identical to SEQ ID NO: 1846, a HCDR1 at least 65%, at least 70%, at least 75%, at least 80%, at least 85%, at least 90%, at least 95%, at least 99%, or 100% identical to SEQ ID NO: 1848, a HCDR2 at least 65%, at least 70%, at least 75%, at least 80%, at least 85%, at least 90%, at least 95%, at least 99%, or 100% identical to SEQ ID NO: 1849, and a HCDR3 at least 65%, at least 70%, at least 75%, at least 80%, at least 85%, at least 90%, at least 95%, at least 99%, or 100% identical to SEQ ID NO: 1850;
a LCDR1 at least 65%, at least 70%, at least 75%, at least 80%, at least 85%, at least 90%, at least 95%, at least 99%, or 100% identical to SEQ ID NO: 1852, a LCDR2 at least 65%, at least 70%, at least 75%, at least 80%, at least 85%, at least 90%, at least 95%, at least 99%, or 100% identical to amino acid sequence GAS, a LCDR3 at least 65%, at least 70%, at least 75%, at least 80%, at least 85%, at least 90%, at least 95%, at least 99%, or 100% identical to SEQ ID NO: 1853, a HCDR1 at least 65%, at least 70%, at least 75%, at least 80%, at least 85%, at least 90%, at least 95%, at least 99%, or 100% identical to SEQ ID NO: 1855, a HCDR2 at least 65%, at least 70%, at least 75%, at least 80%, at least 85%, at least 90%, at least 95%, at least 99%, or 100% identical to SEQ ID NO: 1856, and a HCDR3 at least 65%, at least 70%, at least 75%, at least 80%, at least 85%, at least 90%, at least 95%, at least 99%, or 100% identical to SEQ ID NO: 1857;
a LCDR1 at least 65%, at least 70%, at least 75%, at least 80%, at least 85%, at least 90%, at least 95%, at least 99%, or 100% identical to SEQ ID NO: 1859, a LCDR2 at least 65%, at least 70%, at least 75%, at least 80%, at least 85%, at least 90%, at least 95%, at least 99%, or 100% identical to amino acid sequence GNG, a LCDR3 at least 65%, at least 70%, at least 75%, at least 80%, at least 85%, at least 90%, at least 95%, at least 99%, or 100% identical to SEQ ID NO: 1860, a HCDR1 at least 65%, at least 70%, at least 75%, at least 80%, at least 85%, at least 90%, at least 95%, at least 99%, or 100% identical to SEQ ID NO: 1862, a HCDR2 at least 65%, at least 70%, at least 75%, at least 80%, at least 85%, at least 90%, at least 95%, at least 99%, or 100% identical to SEQ ID NO: 1863, and a HCDR3 at least 65%, at least 70%, at least 75%, at least 80%, at least 85%, at least 90%, at least 95%, at least 99%, or 100% identical to SEQ ID NO: 1864;
a LCDR1 at least 65%, at least 70%, at least 75%, at least 80%, at least 85%, at least 90%, at least 95%, at least 99%, or 100% identical to SEQ ID NO: 1359, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1360, a HCDR1 at least 65%, at least 70%, at least 75%, at least 80%, at least 85%, at least 90%, at least 95%, at least 99%, or 100% identical to SEQ ID NO: 1362, a HCDR2 at least 65%, at least 70%, at least 75%, at least 80%, at least 85%, at least 90%, at least 95%, at least 99%, or 100% identical to SEQ ID NO: 1363, and a HCDR3 at least 65%, at least 70%, at least 75%, at least 80%, at least 85%, at least 90%, at least 95%, at least 99%, or 100% identical to SEQ ID NO: 1364;
a LCDR1 at least 65%, at least 70%, at least 75%, at least 80%, at least 85%, at least 90%, at least 95%, at least 99%, or 100% identical to SEQ ID NO: 1866, a LCDR2 at least 65%, at least 70%, at least 75%, at least 80%, at least 85%, at least 90%, at least 95%, at least 99%, or 100% identical to amino acid sequence LVS, a LCDR3 at least 65%, at least 70%, at least 75%, at least 80%, at least 85%, at least 90%, at least 95%, at least 99%, or 100% identical to SEQ ID NO: 1867, a HCDR1 at least 65%, at least 70%, at least 75%, at least 80%, at least 85%, at least 90%, at least 95%, at least 99%, or 100% identical to SEQ ID NO: 1309, a HCDR2 at least 65%, at least 70%, at least 75%, at least 80%, at least 85%, at least 90%, at least 95%, at least 99%, or 100% identical to SEQ ID NO: 1869, and a HCDR3 at least 65%, at least 70%, at least 75%, at least 80%, at least 85%, at least 90%, at least 95%, at least 99%, or 100% identical to SEQ ID NO: 1870;
a LCDR1 at least 65%, at least 70%, at least 75%, at least 80%, at least 85%, at least 90%, at least 95%, at least 99%, or 100% identical to SEQ ID NO: 1872, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1873, a HCDR1 at least 65%, at least 70%, at least 75%, at least 80%, at least 85%, at least 90%, at least 95%, at least 99%, or 100% identical to SEQ ID NO: 1552, a HCDR2 at least 65%, at least 70%, at least 75%, at least 80%, at least 85%, at least 90%, at least 95%, at least 99%, or 100% identical to SEQ ID NO: 1875, and a HCDR3 at least 65%, at least 70%, at least 75%, at least 80%, at least 85%, at least 90%, at least 95%, at least 99%, or 100% identical to SEQ ID NO: 1876;
a LCDR1 at least 65%, at least 70%, at least 75%, at least 80%, at least 85%, at least 90%, at least 95%, at least 99%, or 100% identical to SEQ ID NO: 1878, a LCDR2 at least 65%, at least 70%, at least 75%, at least 80%, at least 85%, at least 90%, at least 95%, at least 99%, or 100% identical to amino acid sequence AVS, a LCDR3 at least 65%, at least 70%, at least 75%, at least 80%, at least 85%, at least 90%, at least 95%, at least 99%, or 100% identical to SEQ ID NO: 1879, a HCDR1 at least 65%, at least 70%, at least 75%, at least 80%, at least 85%, at least 90%, at least 95%, at least 99%, or 100% identical to SEQ ID NO: 1881, a HCDR2 at least 65%, at least 70%, at least 75%, at least 80%, at least 85%, at least 90%, at least 95%, at least 99%, or 100% identical to SEQ ID NO: 1882, and a HCDR3 at least 65%, at least 70%, at least 75%, at least 80%, at least 85%, at least 90%, at least 95%, at least 99%, or 100% identical to SEQ ID NO: 1883;
a LCDR1 at least 65%, at least 70%, at least 75%, at least 80%, at least 85%, at least 90%, at least 95%, at least 99%, or 100% identical to SEQ ID NO: 1885, a LCDR2 at least 65%, at least 70%, at least 75%, at least 80%, at least 85%, at least 90%, at least 95%, at least 99%, or 100% identical to amino acid sequence EDN, a LCDR3 at least 65%, at least 70%, at least 75%, at least 80%, at least 85%, at least 90%, at least 95%, at least 99%, or 100% identical to SEQ ID NO: 1886, a HCDR1 at least 65%, at least 70%, at least 75%, at least 80%, at least 85%, at least 90%, at least 95%, at least 99%, or 100% identical to SEQ ID NO: 1618, a HCDR2 at least 65%, at least 70%, at least 75%, at least 80%, at least 85%, at least 90%, at least 95%, at least 99%, or 100% identical to SEQ ID NO: 1888, and a HCDR3 at least 65%, at least 70%, at least 75%, at least 80%, at least 85%, at least 90%, at least 95%, at least 99%, or 100% identical to SEQ ID NO: 1889;
a LCDR1 at least 65%, at least 70%, at least 75%, at least 80%, at least 85%, at least 90%, at least 95%, at least 99%, or 100% identical to SEQ ID NO: 1891, a LCDR2 at least 65%, at least 70%, at least 75%, at least 80%, at least 85%, at least 90%, at least 95%, at least 99%, or 100% identical to amino acid sequence DAS, a LCDR3 at least 65%, at least 70%, at least 75%, at least 80%, at least 85%, at least 90%, at least 95%, at least 99%, or 100% identical to SEQ ID NO: 1892, a HCDR1 at least 65%, at least 70%, at least 75%, at least 80%, at least 85%, at least 90%, at least 95%, at least 99%, or 100% identical to SEQ ID NO: 1894, a HCDR2 at least 65%, at least 70%, at least 75%, at least 80%, at least 85%, at least 90%, at least 95%, at least 99%, or 100% identical to SEQ ID NO: 1895, and a HCDR3 at least 65%, at least 70%, at least 75%, at least 80%, at least 85%, at least 90%, at least 95%, at least 99%, or 100% identical to SEQ ID NO: 1896;
a LCDR1 at least 65%, at least 70%, at least 75%, at least 80%, at least 85%, at least 90%, at least 95%, at least 99%, or 100% identical to SEQ ID NO: 1898, a LCDR2 at least 65%, at least 70%, at least 75%, at least 80%, at least 85%, at least 90%, at least 95%, at least 99%, or 100% identical to amino acid sequence DNN, a LCDR3 at least 65%, at least 70%, at least 75%, at least 80%, at least 85%, at least 90%, at least 95%, at least 99%, or 100% identical to SEQ ID NO: 1899, a HCDR1 at least 65%, at least 70%, at least 75%, at least 80%, at least 85%, at least 90%, at least 95%, at least 99%, or 100% identical to SEQ ID NO: 1901, a HCDR2 at least 65%, at least 70%, at least 75%, at least 80%, at least 85%, at least 90%, at least 95%, at least 99%, or 100% identical to SEQ ID NO: 1902, and a HCDR3 at least 65%, at least 70%, at least 75%, at least 80%, at least 85%, at least 90%, at least 95%, at least 99%, or 100% identical to SEQ ID NO: 1903;
a LCDR1 at least 65%, at least 70%, at least 75%, at least 80%, at least 85%, at least 90%, at least 95%, at least 99%, or 100% identical to SEQ ID NO: 1905, a LCDR2 at least 65%, at least 70%, at least 75%, at least 80%, at least 85%, at least 90%, at least 95%, at least 99%, or 100% identical to amino acid sequence KAS, a LCDR3 at least 65%, at least 70%, at least 75%, at least 80%, at least 85%, at least 90%, at least 95%, at least 99%, or 100% identical to SEQ ID NO: 1906, a HCDR1 at least 65%, at least 70%, at least 75%, at least 80%, at least 85%, at least 90%, at least 95%, at least 99%, or 100% identical to SEQ ID NO: 1639, a HCDR2 at least 65%, at least 70%, at least 75%, at least 80%, at least 85%, at least 90%, at least 95%, at least 99%, or 100% identical to SEQ ID NO: 1908, and a HCDR3 at least 65%, at least 70%, at least 75%, at least 80%, at least 85%, at least 90%, at least 95%, at least 99%, or 100% identical to SEQ ID NO: 1909;
a LCDR1 at least 65%, at least 70%, at least 75%, at least 80%, at least 85%, at least 90%, at least 95%, at least 99%, or 100% identical to SEQ ID NO: 1911, a LCDR2 at least 65%, at least 70%, at least 75%, at least 80%, at least 85%, at least 90%, at least 95%, at least 99%, or 100% identical to amino acid sequence GAS, a LCDR3 at least 65%, at least 70%, at least 75%, at least 80%, at least 85%, at least 90%, at least 95%, at least 99%, or 100% identical to SEQ ID NO: 1912, a HCDR1 at least 65%, at least 70%, at least 75%, at least 80%, at least 85%, at least 90%, at least 95%, at least 99%, or 100% identical to SEQ ID NO: 1437, a HCDR2 at least 65%, at least 70%, at least 75%, at least 80%, at least 85%, at least 90%, at least 95%, at least 99%, or 100% identical to SEQ ID NO: 1914, and a HCDR3 at least 65%, at least 70%, at least 75%, at least 80%, at least 85%, at least 90%, at least 95%, at least 99%, or 100% identical to SEQ ID NO: 1915;
a LCDR1 at least 65%, at least 70%, at least 75%, at least 80%, at least 85%, at least 90%, at least 95%, at least 99%, or 100% identical to SEQ ID NO: 1917, a LCDR2 at least 65%, at least 70%, at least 75%, at least 80%, at least 85%, at least 90%, at least 95%, at least 99%, or 100% identical to amino acid sequence NTD, a LCDR3 at least 65%, at least 70%, at least 75%, at least 80%, at least 85%, at least 90%, at least 95%, at least 99%, or 100% identical to SEQ ID NO: 1918, a HCDR1 at least 65%, at least 70%, at least 75%, at least 80%, at least 85%, at least 90%, at least 95%, at least 99%, or 100% identical to SEQ ID NO: 1920, a HCDR2 at least 65%, at least 70%, at least 75%, at least 80%, at least 85%, at least 90%, at least 95%, at least 99%, or 100% identical to SEQ ID NO: 1921, and a HCDR3 at least 65%, at least 70%, at least 75%, at least 80%, at least 85%, at least 90%, at least 95%, at least 99%, or 100% identical to SEQ ID NO: 1922;
a LCDR1 at least 65%, at least 70%, at least 75%, at least 80%, at least 85%, at least 90%, at least 95%, at least 99%, or 100% identical to SEQ ID NO: 1924, a LCDR2 at least 65%, at least 70%, at least 75%, at least 80%, at least 85%, at least 90%, at least 95%, at least 99%, or 100% identical to amino acid sequence YTS, a LCDR3 at least 65%, at least 70%, at least 75%, at least 80%, at least 85%, at least 90%, at least 95%, at least 99%, or 100% identical to SEQ ID NO: 1925, a HCDR1 at least 65%, at least 70%, at least 75%, at least 80%, at least 85%, at least 90%, at least 95%, at least 99%, or 100% identical to SEQ ID NO: 1927, a HCDR2 at least 65%, at least 70%, at least 75%, at least 80%, at least 85%, at least 90%, at least 95%, at least 99%, or 100% identical to SEQ ID NO: 1928, and a HCDR3 at least 65%, at least 70%, at least 75%, at least 80%, at least 85%, at least 90%, at least 95%, at least 99%, or 100% identical to SEQ ID NO: 1929;
a LCDR1 at least 65%, at least 70%, at least 75%, at least 80%, at least 85%, at least 90%, at least 95%, at least 99%, or 100% identical to SEQ ID NO: 1931, a LCDR2 at least 65%, at least 70%, at least 75%, at least 80%, at least 85%, at least 90%, at least 95%, at least 99%, or 100% identical to amino acid sequence LVS, a LCDR3 at least 65%, at least 70%, at least 75%, at least 80%, at least 85%, at least 90%, at least 95%, at least 99%, or 100% identical to SEQ ID NO: 1932, a HCDR1 at least 65%, at least 70%, at least 75%, at least 80%, at least 85%, at least 90%, at least 95%, at least 99%, or 100% identical to SEQ ID NO: 1934, a HCDR2 at least 65%, at least 70%, at least 75%, at least 80%, at least 85%, at least 90%, at least 95%, at least 99%, or 100% identical to SEQ ID NO: 1935, and a HCDR3 at least 65%, at least 70%, at least 75%, at least 80%, at least 85%, at least 90%, at least 95%, at least 99%, or 100% identical to SEQ ID NO: 1936;
a LCDR1 at least 65%, at least 70%, at least 75%, at least 80%, at least 85%, at least 90%, at least 95%, at least 99%, or 100% identical to SEQ ID NO: 1938, a LCDR2 at least 65%, at least 70%, at least 75%, at least 80%, at least 85%, at least 90%, at least 95%, at least 99%, or 100% identical to amino acid sequence KAS, a LCDR3 at least 65%, at least 70%, at least 75%, at least 80%, at least 85%, at least 90%, at least 95%, at least 99%, or 100% identical to SEQ ID NO: 1939, a HCDR1 at least 65%, at least 70%, at least 75%, at least 80%, at least 85%, at least 90%, at least 95%, at least 99%, or 100% identical to SEQ ID NO: 1941, a HCDR2 at least 65%, at least 70%, at least 75%, at least 80%, at least 85%, at least 90%, at least 95%, at least 99%, or 100% identical to SEQ ID NO: 1942, and a HCDR3 at least 65%, at least 70%, at least 75%, at least 80%, at least 85%, at least 90%, at least 95%, at least 99%, or 100% identical to SEQ ID NO: 1943;
a LCDR1 at least 65%, at least 70%, at least 75%, at least 80%, at least 85%, at least 90%, at least 95%, at least 99%, or 100% identical to SEQ ID NO: 1945, a LCDR2 at least 65%, at least 70%, at least 75%, at least 80%, at least 85%, at least 90%, at least 95%, at least 99%, or 100% identical to amino acid sequence KVS, a LCDR3 at least 65%, at least 70%, at least 75%, at least 80%, at least 85%, at least 90%, at least 95%, at least 99%, or 100% identical to SEQ ID NO: 1946, a HCDR1 at least 65%, at least 70%, at least 75%, at least 80%, at least 85%, at least 90%, at least 95%, at least 99%, or 100% identical to SEQ ID NO: 1901, a HCDR2 at least 65%, at least 70%, at least 75%, at least 80%, at least 85%, at least 90%, at least 95%, at least 99%, or 100% identical to SEQ ID NO: 1948, and a HCDR3 at least 65%, at least 70%, at least 75%, at least 80%, at least 85%, at least 90%, at least 95%, at least 99%, or 100% identical to SEQ ID NO: 1949;
a LCDR1 at least 65%, at least 70%, at least 75%, at least 80%, at least 85%, at least 90%, at least 95%, at least 99%, or 100% identical to SEQ ID NO: 1120, a LCDR2 at least 65%, at least 70%, at least 75%, at least 80%, at least 85%, at least 90%, at least 95%, at least 99%, or 100% identical to amino acid sequence AAS, a LCDR3 at least 65%, at least 70%, at least 75%, at least 80%, at least 85%, at least 90%, at least 95%, at least 99%, or 100% identical to SEQ ID NO: 1951, a HCDR1 at least 65%, at least 70%, at least 75%, at least 80%, at least 85%, at least 90%, at least 95%, at least 99%, or 100% identical to SEQ ID NO: 1953, a HCDR2 at least 65%, at least 70%, at least 75%, at least 80%, at least 85%, at least 90%, at least 95%, at least 99%, or 100% identical to SEQ ID NO: 1954, and a HCDR3 at least 65%, at least 70%, at least 75%, at least 80%, at least 85%, at least 90%, at least 95%, at least 99%, or 100% identical to SEQ ID NO: 1955;
a LCDR1 at least 65%, at least 70%, at least 75%, at least 80%, at least 85%, at least 90%, at least 95%, at least 99%, or 100% identical to SEQ ID NO: 1228, a LCDR2 at least 65%, at least 70%, at least 75%, at least 80%, at least 85%, at least 90%, at least 95%, at least 99%, or 100% identical to amino acid sequence YTS, a LCDR3 at least 65%, at least 70%, at least 75%, at least 80%, at least 85%, at least 90%, at least 95%, at least 99%, or 100% identical to SEQ ID NO: 1957, a HCDR1 at least 65%, at least 70%, at least 75%, at least 80%, at least 85%, at least 90%, at least 95%, at least 99%, or 100% identical to SEQ ID NO: 1959, a HCDR2 at least 65%, at least 70%, at least 75%, at least 80%, at least 85%, at least 90%, at least 95%, at least 99%, or 100% identical to SEQ ID NO: 1960, and a HCDR3 at least 65%, at least 70%, at least 75%, at least 80%, at least 85%, at least 90%, at least 95%, at least 99%, or 100% identical to SEQ ID NO: 1961;
a LCDR1 at least 65%, at least 70%, at least 75%, at least 80%, at least 85%, at least 90%, at least 95%, at least 99%, or 100% identical to SEQ ID NO: 1963, a LCDR2 at least 65%, at least 70%, at least 75%, at least 80%, at least 85%, at least 90%, at least 95%, at least 99%, or 100% identical to amino acid sequence LVS, a LCDR3 at least 65%, at least 70%, at least 75%, at least 80%, at least 85%, at least 90%, at least 95%, at least 99%, or 100% identical to SEQ ID NO: 1964, a HCDR1 at least 65%, at least 70%, at least 75%, at least 80%, at least 85%, at least 90%, at least 95%, at least 99%, or 100% identical to SEQ ID NO: 1966, a HCDR2 at least 65%, at least 70%, at least 75%, at least 80%, at least 85%, at least 90%, at least 95%, at least 99%, or 100% identical to SEQ ID NO: 1967, and a HCDR3 at least 65%, at least 70%, at least 75%, at least 80%, at least 85%, at least 90%, at least 95%, at least 99%, or 100% identical to SEQ ID NO: 1968; or
a LCDR1 at least 65%, at least 70%, at least 75%, at least 80%, at least 85%, at least 90%, at least 95%, at least 99%, or 100% identical to SEQ ID NO: 1519, a LCDR2 at least 65%, at least 70%, at least 75%, at least 80%, at least 85%, at least 90%, at least 95%, at least 99%, or 100% identical to amino acid sequence STS, a LCDR3 at least 65%, at least 70%, at least 75%, at least 80%, at least 85%, at least 90%, at least 95%, at least 99%, or 100% identical to SEQ ID NO: 1970, a HCDR1 at least 65%, at least 70%, at least 75%, at least 80%, at least 85%, at least 90%, at least 95%, at least 99%, or 100% identical to SEQ ID NO: 1309, a HCDR2 at least 65%, at least 70%, at least 75%, at least 80%, at least 85%, at least 90%, at least 95%, at least 99%, or 100% identical to SEQ ID NO: 1972, and a HCDR3 at least 65%, at least 70%, at least 75%, at least 80%, at least 85%, at least 90%, at least 95%, at least 99%, or 100% identical to SEQ ID NO: 1973.

In some aspects, any of the antigen binding polypeptides, antigen binding polypeptide complexes, or antibodies or antigen binding fragments thereof disclosed herein comprises:
a VL at least 65%, at least 70%, at least 75%, at least 80%, at least 85%, at least 90%, at least 95%, at least 99%, or 100% identical to SEQ ID NO: 1098 and a VH at least 65%, at least 70%, at least 75%, at least 80%, at least 85%, at least 90%, at least 95%, at least 99%, or 100% identical to SEQ ID NO: 1102;
a VL at least 65%, at least 70%, at least 75%, at least 80%, at least 85%, at least 90%, at least 95%, at least 99%, or 100% identical to SEQ ID NO: 1105 and a VH at least 65%, at least 70%, at least 75%, at least 80%, at least 85%, at least 90%, at least 95%, at least 99%, or 100% identical to SEQ ID NO: 1109;
a VL at least 65%, at least 70%, at least 75%, at least 80%, at least 85%, at least 90%, at least 95%, at least 99%, or 100% identical to SEQ ID NO: 1112 and a VH at least 65%, at least 70%, at least 75%, at least 80%, at least 85%, at least 90%, at least 95%, at least 99%, or 100% identical to SEQ ID NO: 1116;
a VL at least 65%, at least 70%, at least 75%, at least 80%, at least 85%, at least 90%, at least 95%, at least 99%, or 100% identical to SEQ ID NO: 1105 and a VH at least 65%, at least 70%, at least 75%, at least 80%, at least 85%, at least 90%, at least 95%, at least 99%, or 100% identical to SEQ ID NO: 1119;
a VL at least 65%, at least 70%, at least 75%, at least 80%, at least 85%, at least 90%, at least 95%, at least 99%, or 100% identical to SEQ ID NO: 1122 and a VH at least 65%, at least 70%, at least 75%, at least 80%, at least 85%, at least 90%, at least 95%, at least 99%, or 100% identical to SEQ ID NO: 1126;
a VL at least 65%, at least 70%, at least 75%, at least 80%, at least 85%, at least 90%, at least 95%, at least 99%, or 100% identical to SEQ ID NO: 1129 and a VH at least 65%, at least 70%, at least 75%, at least 80%, at least 85%, at least 90%, at least 95%, at least 99%, or 100% identical to SEQ ID NO: 1133;
a VL at least 65%, at least 70%, at least 75%, at least 80%, at least 85%, at least 90%, at least 95%, at least 99%, or 100% identical to SEQ ID NO: 1136 and a VH at least 65%, at least 70%, at least 75%, at least 80%, at least 85%, at least 90%, at least 95%, at least 99%, or 100% identical to SEQ ID NO: 1140;
a VL at least 65%, at least 70%, at least 75%, at least 80%, at least 85%, at least 90%, at least 95%, at least 99%, or 100% identical to SEQ ID NO: 1143 and a VH at least 65%, at least 70%, at least 75%, at least 80%, at least 85%, at least 90%, at least 95%, at least 99%, or 100% identical to SEQ ID NO: 1147;
a VL at least 65%, at least 70%, at least 75%, at least 80%, at least 85%, at least 90%, at least 95%, at least 99%, or 100% identical to SEQ ID NO: 1150 and a VH at least 65%, at least 70%, at least 75%, at least 80%, at least 85%, at least 90%, at least 95%, at least 99%, or 100% identical to SEQ ID NO: 1154;
a VL at least 65%, at least 70%, at least 75%, at least 80%, at least 85%, at least 90%, at least 95%, at least 99%, or 100% identical to SEQ ID NO: 1157 and a VH at least 65%, at least 70%, at least 75%, at least 80%, at least 85%, at least 90%, at least 95%, at least 99%, or 100% identical to SEQ ID NO: 1161;
a VL at least 65%, at least 70%, at least 75%, at least 80%, at least 85%, at least 90%, at least 95%, at least 99%, or 100% identical to SEQ ID NO: 1164 and a VH at least 65%, at least 70%, at least 75%, at least 80%, at least 85%, at least 90%, at least 95%, at least 99%, or 100% identical to SEQ ID NO: 1168;
a VL at least 65%, at least 70%, at least 75%, at least 80%, at least 85%, at least 90%, at least 95%, at least 99%, or 100% identical to SEQ ID NO: 1171 and a VH at least 65%, at least 70%, at least 75%, at least 80%, at least 85%, at least 90%, at least 95%, at least 99%, or 100% identical to SEQ ID NO: 1173;
a VL at least 65%, at least 70%, at least 75%, at least 80%, at least 85%, at least 90%, at least 95%, at least 99%, or 100% identical to SEQ ID NO: 1176 and a VH at least 65%, at least 70%, at least 75%, at least 80%, at least 85%, at least 90%, at least 95%, at least 99%, or 100% identical to SEQ ID NO: 1180;
a VL at least 65%, at least 70%, at least 75%, at least 80%, at least 85%, at least 90%, at least 95%, at least 99%, or 100% identical to SEQ ID NO: 1183 and a VH at least 65%, at least 70%, at least 75%, at least 80%, at least 85%, at least 90%, at least 95%, at least 99%, or 100% identical to SEQ ID NO: 1187;
a VL at least 65%, at least 70%, at least 75%, at least 80%, at least 85%, at least 90%, at least 95%, at least 99%, or 100% identical to SEQ ID NO: 1190 and a VH at least 65%, at least 70%, at least 75%, at least 80%, at least 85%, at least 90%, at least 95%, at least 99%, or 100% identical to SEQ ID NO: 1194;
a VL at least 65%, at least 70%, at least 75%, at least 80%, at least 85%, at least 90%, at least 95%, at least 99%, or 100% identical to SEQ ID NO: 1196 and a VH at least 65%, at least 70%, at least 75%, at least 80%, at least 85%, at least 90%, at least 95%, at least 99%, or 100% identical to SEQ ID NO: 1198;
a VL at least 65%, at least 70%, at least 75%, at least 80%, at least 85%, at least 90%, at least 95%, at least 99%, or 100% identical to SEQ ID NO: 1202 and a VH at least 65%, at least 70%, at least 75%, at least 80%, at least 85%, at least 90%, at least 95%, at least 99%, or 100% identical to SEQ ID NO: 1206;
a VL at least 65%, at least 70%, at least 75%, at least 80%, at least 85%, at least 90%, at least 95%, at least 99%, or 100% identical to SEQ ID NO: 1208 and a VH at least 65%, at least 70%, at least 75%, at least 80%, at least 85%, at least 90%, at least 95%, at least 99%, or 100% identical to SEQ ID NO: 1210;
a VL at least 65%, at least 70%, at least 75%, at least 80%, at least 85%, at least 90%, at least 95%, at least 99%, or 100% identical to SEQ ID NO: 1214 and a VH at least 65%, at least 70%, at least 75%, at least 80%, at least 85%, at least 90%, at least 95%, at least 99%, or 100% identical to SEQ ID NO: 1218;
a VL at least 65%, at least 70%, at least 75%, at least 80%, at least 85%, at least 90%, at least 95%, at least 99%, or 100% identical to SEQ ID NO: 1219 and a VH at least 65%, at least 70%, at least 75%, at least 80%, at least 85%, at least 90%, at least 95%, at least 99%, or 100% identical to SEQ ID NO: 1220;
a VL at least 65%, at least 70%, at least 75%, at least 80%, at least 85%, at least 90%, at least 95%, at least 99%, or 100% identical to SEQ ID NO: 1223 and a VH at least 65%, at least 70%, at least 75%, at least 80%, at least 85%, at least 90%, at least 95%, at least 99%, or 100% identical to SEQ ID NO: 1227;
a VL at least 65%, at least 70%, at least 75%, at least 80%, at least 85%, at least 90%, at least 95%, at least 99%, or 100% identical to SEQ ID NO: 1230 and a VH at least 65%, at least 70%, at least 75%, at least 80%, at least 85%, at least 90%, at least 95%, at least 99%, or 100% identical to SEQ ID NO: 1234;
a VL at least 65%, at least 70%, at least 75%, at least 80%, at least 85%, at least 90%, at least 95%, at least 99%, or 100% identical to SEQ ID NO: 1237 and a VH at least 65%, at least 70%, at least 75%, at least 80%, at least 85%, at least 90%, at least 95%, at least 99%, or 100% identical to SEQ ID NO: 1241;
a VL at least 65%, at least 70%, at least 75%, at least 80%, at least 85%, at least 90%, at least 95%, at least 99%, or 100% identical to SEQ ID NO: 1242 and a VH at least 65%, at least 70%, at least 75%, at least 80%, at least 85%, at least 90%, at least 95%, at least 99%, or 100% identical to SEQ ID NO: 1246;
a VL at least 65%, at least 70%, at least 75%, at least 80%, at least 85%, at least 90%, at least 95%, at least 99%, or 100% identical to SEQ ID NO: 1249 and a VH at least 65%, at least 70%, at least 75%, at least 80%, at least 85%, at least 90%, at least 95%, at least 99%, or 100% identical to SEQ ID NO: 1253;
a VL at least 65%, at least 70%, at least 75%, at least 80%, at least 85%, at least 90%, at least 95%, at least 99%, or 100% identical to SEQ ID NO: 1256 and a VH at least 65%, at least 70%, at least 75%, at least 80%, at least 85%, at least 90%, at least 95%, at least 99%, or 100% identical to SEQ ID NO: 1260;
a VL at least 65%, at least 70%, at least 75%, at least 80%, at least 85%, at least 90%, at least 95%, at least 99%, or 100% identical to SEQ ID NO: 1263 and a VH at least 65%, at least 70%, at least 75%, at least 80%, at least 85%, at least 90%, at least 95%, at least 99%, or 100% identical to SEQ ID NO: 1265;
a VL at least 65%, at least 70%, at least 75%, at least 80%, at least 85%, at least 90%, at least 95%, at least 99%, or 100% identical to SEQ ID NO: 1268 and a VH at least 65%, at least 70%, at least 75%, at least 80%, at least 85%, at least 90%, at least 95%, at least 99%, or 100% identical to SEQ ID NO: 1270;
a VL at least 65%, at least 70%, at least 75%, at least 80%, at least 85%, at least 90%, at least 95%, at least 99%, or 100% identical to SEQ ID NO: 1273 and a VH at least 65%, at least 70%, at least 75%, at least 80%, at least 85%, at least 90%, at least 95%, at least 99%, or 100% identical to SEQ ID NO: 1277;
a VL at least 65%, at least 70%, at least 75%, at least 80%, at least 85%, at least 90%, at least 95%, at least 99%, or 100% identical to SEQ ID NO: 1280 and a VH at least 65%, at least 70%, at least 75%, at least 80%, at least 85%, at least 90%, at least 95%, at least 99%, or 100% identical to SEQ ID NO: 1284;
a VL at least 65%, at least 70%, at least 75%, at least 80%, at least 85%, at least 90%, at least 95%, at least 99%, or 100% identical to SEQ ID NO: 1287 and a VH at least 65%, at least 70%, at least 75%, at least 80%, at least 85%, at least 90%, at least 95%, at least 99%, or 100% identical to SEQ ID NO: 1291;
a VL at least 65%, at least 70%, at least 75%, at least 80%, at least 85%, at least 90%, at least 95%, at least 99%, or 100% identical to SEQ ID NO: 1294 and a VH at least 65%, at least 70%, at least 75%, at least 80%, at least 85%, at least 90%, at least 95%, at least 99%, or 100% identical to SEQ ID NO: 1298;
a VL at least 65%, at least 70%, at least 75%, at least 80%, at least 85%, at least 90%, at least 95%, at least 99%, or 100% identical to SEQ ID NO: 1301 and a VH at least 65%, at least 70%, at least 75%, at least 80%, at least 85%, at least 90%, at least 95%, at least 99%, or 100% identical to SEQ ID NO: 1305;
a VL at least 65%, at least 70%, at least 75%, at least 80%, at least 85%, at least 90%, at least 95%, at least 99%, or 100% identical to SEQ ID NO: 1308 and a VH at least 65%, at least 70%, at least 75%, at least 80%, at least 85%, at least 90%, at least 95%, at least 99%, or 100% identical to SEQ ID NO: 1312;
a VL at least 65%, at least 70%, at least 75%, at least 80%, at least 85%, at least 90%, at least 95%, at least 99%, or 100% identical to SEQ ID NO: 1314 and a VH at least 65%, at least 70%, at least 75%, at least 80%, at least 85%, at least 90%, at least 95%, at least 99%, or 100% identical to SEQ ID NO: 1318;
a VL at least 65%, at least 70%, at least 75%, at least 80%, at least 85%, at least 90%, at least 95%, at least 99%, or 100% identical to SEQ ID NO: 1321 and a VH at least 65%, at least 70%, at least 75%, at least 80%, at least 85%, at least 90%, at least 95%, at least 99%, or 100% identical to SEQ ID NO: 1325;
a VL at least 65%, at least 70%, at least 75%, at least 80%, at least 85%, at least 90%, at least 95%, at least 99%, or 100% identical to SEQ ID NO: 1328 and a VH at least 65%, at least 70%, at least 75%, at least 80%, at least 85%, at least 90%, at least 95%, at least 99%, or 100% identical to SEQ ID NO: 1332;
a VL at least 65%, at least 70%, at least 75%, at least 80%, at least 85%, at least 90%, at least 95%, at least 99%, or 100% identical to SEQ ID NO: 1334 and a VH at least 65%, at least 70%, at least 75%, at least 80%, at least 85%, at least 90%, at least 95%, at least 99%, or 100% identical to SEQ ID NO: 1338;
a VL at least 65%, at least 70%, at least 75%, at least 80%, at least 85%, at least 90%, at least 95%, at least 99%, or 100% identical to SEQ ID NO: 1341 and a VH at least 65%, at least 70%, at least 75%, at least 80%, at least 85%, at least 90%, at least 95%, at least 99%, or 100% identical to SEQ ID NO: 1345;
a VL at least 65%, at least 70%, at least 75%, at least 80%, at least 85%, at least 90%, at least 95%, at least 99%, or 100% identical to SEQ ID NO: 1347 and a VH at least 65%, at least 70%, at least 75%, at least 80%, at least 85%, at least 90%, at least 95%, at least 99%, or 100% identical to SEQ ID NO: 1351;
a VL at least 65%, at least 70%, at least 75%, at least 80%, at least 85%, at least 90%, at least 95%, at least 99%, or 100% identical to SEQ ID NO: 1354 and a VH at least 65%, at least 70%, at least 75%, at least 80%, at least 85%, at least 90%, at least 95%, at least 99%, or 100% identical to SEQ ID NO: 1358;
a VL at least 65%, at least 70%, at least 75%, at least 80%, at least 85%, at least 90%, at least 95%, at least 99%, or 100% identical to SEQ ID NO: 1361 and a VH at least 65%, at least 70%, at least 75%, at least 80%, at least 85%, at least 90%, at least 95%, at least 99%, or 100% identical to SEQ ID NO: 1365;
a VL at least 65%, at least 70%, at least 75%, at least 80%, at least 85%, at least 90%, at least 95%, at least 99%, or 100% identical to SEQ ID NO: 1368 and a VH at least 65%, at least 70%, at least 75%, at least 80%, at least 85%, at least 90%, at least 95%, at least 99%, or 100% identical to SEQ ID NO: 1372;
a VL at least 65%, at least 70%, at least 75%, at least 80%, at least 85%, at least 90%, at least 95%, at least 99%, or 100% identical to SEQ ID NO: 1375 and a VH at least 65%, at least 70%, at least 75%, at least 80%, at least 85%, at least 90%, at least 95%, at least 99%, or 100% identical to SEQ ID NO: 1379;
a VL at least 65%, at least 70%, at least 75%, at least 80%, at least 85%, at least 90%, at least 95%, at least 99%, or 100% identical to SEQ ID NO: 1380 and a VH at least 65%, at least 70%, at least 75%, at least 80%, at least 85%, at least 90%, at least 95%, at least 99%, or 100% identical to SEQ ID NO: 1381;
a VL at least 65%, at least 70%, at least 75%, at least 80%, at least 85%, at least 90%, at least 95%, at least 99%, or 100% identical to SEQ ID NO: 1384 and a VH at least 65%, at least 70%, at least 75%, at least 80%, at least 85%, at least 90%, at least 95%, at least 99%, or 100% identical to SEQ ID NO: 1388;
a VL at least 65%, at least 70%, at least 75%, at least 80%, at least 85%, at least 90%, at least 95%, at least 99%, or 100% identical to SEQ ID NO: 1390 and a VH at least 65%, at least 70%, at least 75%, at least 80%, at least 85%, at least 90%, at least 95%, at least 99%, or 100% identical to SEQ ID NO: 1393;
a VL at least 65%, at least 70%, at least 75%, at least 80%, at least 85%, at least 90%, at least 95%, at least 99%, or 100% identical to SEQ ID NO: 1396 and a VH at least 65%, at least 70%, at least 75%, at least 80%, at least 85%, at least 90%, at least 95%, at least 99%, or 100% identical to SEQ ID NO: 1400;
a VL at least 65%, at least 70%, at least 75%, at least 80%, at least 85%, at least 90%, at least 95%, at least 99%, or 100% identical to SEQ ID NO: 1403 and a VH at least 65%, at least 70%, at least 75%, at least 80%, at least 85%, at least 90%, at least 95%, at least 99%, or 100% identical to SEQ ID NO: 1407;
a VL at least 65%, at least 70%, at least 75%, at least 80%, at least 85%, at least 90%, at least 95%, at least 99%, or 100% identical to SEQ ID NO: 1410 and a VH at least 65%, at least 70%, at least 75%, at least 80%, at least 85%, at least 90%, at least 95%, at least 99%, or 100% identical to SEQ ID NO: 1413;
a VL at least 65%, at least 70%, at least 75%, at least 80%, at least 85%, at least 90%, at least 95%, at least 99%, or 100% identical to SEQ ID NO: 1416 and a VH at least 65%, at least 70%, at least 75%, at least 80%, at least 85%, at least 90%, at least 95%, at least 99%, or 100% identical to SEQ ID NO: 1420;
a VL at least 65%, at least 70%, at least 75%, at least 80%, at least 85%, at least 90%, at least 95%, at least 99%, or 100% identical to SEQ ID NO: 1423 and a VH at least 65%, at least 70%, at least 75%, at least 80%, at least 85%, at least 90%, at least 95%, at least 99%, or 100% identical to SEQ ID NO: 1427;
a VL at least 65%, at least 70%, at least 75%, at least 80%, at least 85%, at least 90%, at least 95%, at least 99%, or 100% identical to SEQ ID NO: 1430 and a VH at least 65%, at least 70%, at least 75%, at least 80%, at least 85%, at least 90%, at least 95%, at least 99%, or 100% identical to SEQ ID NO: 1434;
a VL at least 65%, at least 70%, at least 75%, at least 80%, at least 85%, at least 90%, at least 95%, at least 99%, or 100% identical to SEQ ID NO: 1436 and a VH at least 65%, at least 70%, at least 75%, at least 80%, at least 85%, at least 90%, at least 95%, at least 99%, or 100% identical to SEQ ID NO: 1440;
a VL at least 65%, at least 70%, at least 75%, at least 80%, at least 85%, at least 90%, at least 95%, at least 99%, or 100% identical to SEQ ID NO: 1443 and a VH at least 65%, at least 70%, at least 75%, at least 80%, at least 85%, at least 90%, at least 95%, at least 99%, or 100% identical to SEQ ID NO: 1447;
a VL at least 65%, at least 70%, at least 75%, at least 80%, at least 85%, at least 90%, at least 95%, at least 99%, or 100% identical to SEQ ID NO: 1450 and a VH at least 65%, at least 70%, at least 75%, at least 80%, at least 85%, at least 90%, at least 95%, at least 99%, or 100% identical to SEQ ID NO: 1454;
a VL at least 65%, at least 70%, at least 75%, at least 80%, at least 85%, at least 90%, at least 95%, at least 99%, or 100% identical to SEQ ID NO: 1457 and a VH at least 65%, at least 70%, at least 75%, at least 80%, at least 85%, at least 90%, at least 95%, at least 99%, or 100% identical to SEQ ID NO: 1460;
a VL at least 65%, at least 70%, at least 75%, at least 80%, at least 85%, at least 90%, at least 95%, at least 99%, or 100% identical to SEQ ID NO: 1463 and a VH at least 65%, at least 70%, at least 75%, at least 80%, at least 85%, at least 90%, at least 95%, at least 99%, or 100% identical to SEQ ID NO: 1467;
a VL at least 65%, at least 70%, at least 75%, at least 80%, at least 85%, at least 90%, at least 95%, at least 99%, or 100% identical to SEQ ID NO: 1470 and a VH at least 65%, at least 70%, at least 75%, at least 80%, at least 85%, at least 90%, at least 95%, at least 99%, or 100% identical to SEQ ID NO: 1474;
a VL at least 65%, at least 70%, at least 75%, at least 80%, at least 85%, at least 90%, at least 95%, at least 99%, or 100% identical to SEQ ID NO: 1477 and a VH at least 65%, at least 70%, at least 75%, at least 80%, at least 85%, at least 90%, at least 95%, at least 99%, or 100% identical to SEQ ID NO: 1481;
a VL at least 65%, at least 70%, at least 75%, at least 80%, at least 85%, at least 90%, at least 95%, at least 99%, or 100% identical to SEQ ID NO: 1483 and a VH at least 65%, at least 70%, at least 75%, at least 80%, at least 85%, at least 90%, at least 95%, at least 99%, or 100% identical to SEQ ID NO: 1487;
a VL at least 65%, at least 70%, at least 75%, at least 80%, at least 85%, at least 90%, at least 95%, at least 99%, or 100% identical to SEQ ID NO: 1490 and a VH at least 65%, at least 70%, at least 75%, at least 80%, at least 85%, at least 90%, at least 95%, at least 99%, or 100% identical to SEQ ID NO: 1494;
a VL at least 65%, at least 70%, at least 75%, at least 80%, at least 85%, at least 90%, at least 95%, at least 99%, or 100% identical to SEQ ID NO: 1497 and a VH at least 65%, at least 70%, at least 75%, at least 80%, at least 85%, at least 90%, at least 95%, at least 99%, or 100% identical to SEQ ID NO: 1501;
a VL at least 65%, at least 70%, at least 75%, at least 80%, at least 85%, at least 90%, at least 95%, at least 99%, or 100% identical to SEQ ID NO: 1503 and a VH at least 65%, at least 70%, at least 75%, at least 80%, at least 85%, at least 90%, at least 95%, at least 99%, or 100% identical to SEQ ID NO: 1507;
a VL at least 65%, at least 70%, at least 75%, at least 80%, at least 85%, at least 90%, at least 95%, at least 99%, or 100% identical to SEQ ID NO: 1510 and a VH at least 65%, at least 70%, at least 75%, at least 80%, at least 85%, at least 90%, at least 95%, at least 99%, or 100% identical to SEQ ID NO: 1512;
a VL at least 65%, at least 70%, at least 75%, at least 80%, at least 85%, at least 90%, at least 95%, at least 99%, or 100% identical to SEQ ID NO: 1514 and a VH at least 65%, at least 70%, at least 75%, at least 80%, at least 85%, at least 90%, at least 95%, at least 99%, or 100% identical to SEQ ID NO: 1518;
a VL at least 65%, at least 70%, at least 75%, at least 80%, at least 85%, at least 90%, at least 95%, at least 99%, or 100% identical to SEQ ID NO: 1521 and a VH at least 65%, at least 70%, at least 75%, at least 80%, at least 85%, at least 90%, at least 95%, at least 99%, or 100% identical to SEQ ID NO: 1525;
a VL at least 65%, at least 70%, at least 75%, at least 80%, at least 85%, at least 90%, at least 95%, at least 99%, or 100% identical to SEQ ID NO: 1527 and a VH at least 65%, at least 70%, at least 75%, at least 80%, at least 85%, at least 90%, at least 95%, at least 99%, or 100% identical to SEQ ID NO: 1530;
a VL at least 65%, at least 70%, at least 75%, at least 80%, at least 85%, at least 90%, at least 95%, at least 99%, or 100% identical to SEQ ID NO: 1533 and a VH at least 65%, at least 70%, at least 75%, at least 80%, at least 85%, at least 90%, at least 95%, at least 99%, or 100% identical to SEQ ID NO: 1537;
a VL at least 65%, at least 70%, at least 75%, at least 80%, at least 85%, at least 90%, at least 95%, at least 99%, or 100% identical to SEQ ID NO: 1540 and a VH at least 65%, at least 70%, at least 75%, at least 80%, at least 85%, at least 90%, at least 95%, at least 99%, or 100% identical to SEQ ID NO: 1543;
a VL at least 65%, at least 70%, at least 75%, at least 80%, at least 85%, at least 90%, at least 95%, at least 99%, or 100% identical to SEQ ID NO: 1546 and a VH at least 65%, at least 70%, at least 75%, at least 80%, at least 85%, at least 90%, at least 95%, at least 99%, or 100% identical to SEQ ID NO: 1550;
a VL at least 65%, at least 70%, at least 75%, at least 80%, at least 85%, at least 90%, at least 95%, at least 99%, or 100% identical to SEQ ID NO: 1551 and a VH at least 65%, at least 70%, at least 75%, at least 80%, at least 85%, at least 90%, at least 95%, at least 99%, or 100% identical to SEQ ID NO: 1555;
a VL at least 65%, at least 70%, at least 75%, at least 80%, at least 85%, at least 90%, at least 95%, at least 99%, or 100% identical to SEQ ID NO: 1558 and a VH at least 65%, at least 70%, at least 75%, at least 80%, at least 85%, at least 90%, at least 95%, at least 99%, or 100% identical to SEQ ID NO: 1561;
a VL at least 65%, at least 70%, at least 75%, at least 80%, at least 85%, at least 90%, at least 95%, at least 99%, or 100% identical to SEQ ID NO: 1563 and a VH at least 65%, at least 70%, at least 75%, at least 80%, at least 85%, at least 90%, at least 95%, at least 99%, or 100% identical to SEQ ID NO: 1567;
a VL at least 65%, at least 70%, at least 75%, at least 80%, at least 85%, at least 90%, at least 95%, at least 99%, or 100% identical to SEQ ID NO: 1570 and a VH at least 65%, at least 70%, at least 75%, at least 80%, at least 85%, at least 90%, at least 95%, at least 99%, or 100% identical to SEQ ID NO: 1573;
a VL at least 65%, at least 70%, at least 75%, at least 80%, at least 85%, at least 90%, at least 95%, at least 99%, or 100% identical to SEQ ID NO: 1576 and a VH at least 65%, at least 70%, at least 75%, at least 80%, at least 85%, at least 90%, at least 95%, at least 99%, or 100% identical to SEQ ID NO: 1580;
a VL at least 65%, at least 70%, at least 75%, at least 80%, at least 85%, at least 90%, at least 95%, at least 99%, or 100% identical to SEQ ID NO: 1583 and a VH at least 65%, at least 70%, at least 75%, at least 80%, at least 85%, at least 90%, at least 95%, at least 99%, or 100% identical to SEQ ID NO: 1587;
a VL at least 65%, at least 70%, at least 75%, at least 80%, at least 85%, at least 90%, at least 95%, at least 99%, or 100% identical to SEQ ID NO: 1589 and a VH at least 65%, at least 70%, at least 75%, at least 80%, at least 85%, at least 90%, at least 95%, at least 99%, or 100% identical to SEQ ID NO: 1593;
a VL at least 65%, at least 70%, at least 75%, at least 80%, at least 85%, at least 90%, at least 95%, at least 99%, or 100% identical to SEQ ID NO: 1596 and a VH at least 65%, at least 70%, at least 75%, at least 80%, at least 85%, at least 90%, at least 95%, at least 99%, or 100% identical to SEQ ID NO: 1600;
a VL at least 65%, at least 70%, at least 75%, at least 80%, at least 85%, at least 90%, at least 95%, at least 99%, or 100% identical to SEQ ID NO: 1603 and a VH at least 65%, at least 70%, at least 75%, at least 80%, at least 85%, at least 90%, at least 95%, at least 99%, or 100% identical to SEQ ID NO: 1607;
a VL at least 65%, at least 70%, at least 75%, at least 80%, at least 85%, at least 90%, at least 95%, at least 99%, or 100% identical to SEQ ID NO: 1610 and a VH at least 65%, at least 70%, at least 75%, at least 80%, at least 85%, at least 90%, at least 95%, at least 99%, or 100% identical to SEQ ID NO: 1614;
a VL at least 65%, at least 70%, at least 75%, at least 80%, at least 85%, at least 90%, at least 95%, at least 99%, or 100% identical to SEQ ID NO: 1617 and a VH at least 65%, at least 70%, at least 75%, at least 80%, at least 85%, at least 90%, at least 95%, at least 99%, or 100% identical to SEQ ID NO: 1621;
a VL at least 65%, at least 70%, at least 75%, at least 80%, at least 85%, at least 90%, at least 95%, at least 99%, or 100% identical to SEQ ID NO: 1624 and a VH at least 65%, at least 70%, at least 75%, at least 80%, at least 85%, at least 90%, at least 95%, at least 99%, or 100% identical to SEQ ID NO: 1628;
a VL at least 65%, at least 70%, at least 75%, at least 80%, at least 85%, at least 90%, at least 95%, at least 99%, or 100% identical to SEQ ID NO: 1631 and a VH at least 65%, at least 70%, at least 75%, at least 80%, at least 85%, at least 90%, at least 95%, at least 99%, or 100% identical to SEQ ID NO: 1635;
a VL at least 65%, at least 70%, at least 75%, at least 80%, at least 85%, at least 90%, at least 95%, at least 99%, or 100% identical to SEQ ID NO: 1638 and a VH at least 65%, at least 70%, at least 75%, at least 80%, at least 85%, at least 90%, at least 95%, at least 99%, or 100% identical to SEQ ID NO: 1642;
a VL at least 65%, at least 70%, at least 75%, at least 80%, at least 85%, at least 90%, at least 95%, at least 99%, or 100% identical to SEQ ID NO: 1645 and a VH at least 65%, at least 70%, at least 75%, at least 80%, at least 85%, at least 90%, at least 95%, at least 99%, or 100% identical to SEQ ID NO: 1649;
a VL at least 65%, at least 70%, at least 75%, at least 80%, at least 85%, at least 90%, at least 95%, at least 99%, or 100% identical to SEQ ID NO: 1652 and a VH at least 65%, at least 70%, at least 75%, at least 80%, at least 85%, at least 90%, at least 95%, at least 99%, or 100% identical to SEQ ID NO: 1656;
a VL at least 65%, at least 70%, at least 75%, at least 80%, at least 85%, at least 90%, at least 95%, at least 99%, or 100% identical to SEQ ID NO: 1659 and a VH at least 65%, at least 70%, at least 75%, at least 80%, at least 85%, at least 90%, at least 95%, at least 99%, or 100% identical to SEQ ID NO: 1662;
a VL at least 65%, at least 70%, at least 75%, at least 80%, at least 85%, at least 90%, at least 95%, at least 99%, or 100% identical to SEQ ID NO: 1664 and a VH at least 65%, at least 70%, at least 75%, at least 80%, at least 85%, at least 90%, at least 95%, at least 99%, or 100% identical to SEQ ID NO: 1668;
a VL at least 65%, at least 70%, at least 75%, at least 80%, at least 85%, at least 90%, at least 95%, at least 99%, or 100% identical to SEQ ID NO: 1669 and a VH at least 65%, at least 70%, at least 75%, at least 80%, at least 85%, at least 90%, at least 95%, at least 99%, or 100% identical to SEQ ID NO: 1671;
a VL at least 65%, at least 70%, at least 75%, at least 80%, at least 85%, at least 90%, at least 95%, at least 99%, or 100% identical to SEQ ID NO: 1674 and a VH at least 65%, at least 70%, at least 75%, at least 80%, at least 85%, at least 90%, at least 95%, at least 99%, or 100% identical to SEQ ID NO: 1678;
a VL at least 65%, at least 70%, at least 75%, at least 80%, at least 85%, at least 90%, at least 95%, at least 99%, or 100% identical to SEQ ID NO: 1682 and a VH at least 65%, at least 70%, at least 75%, at least 80%, at least 85%, at least 90%, at least 95%, at least 99%, or 100% identical to SEQ ID NO: 1686;
a VL at least 65%, at least 70%, at least 75%, at least 80%, at least 85%, at least 90%, at least 95%, at least 99%, or 100% identical to SEQ ID NO: 1692 and a VH at least 65%, at least 70%, at least 75%, at least 80%, at least 85%, at least 90%, at least 95%, at least 99%, or 100% identical to SEQ ID NO: 1696;
a VL at least 65%, at least 70%, at least 75%, at least 80%, at least 85%, at least 90%, at least 95%, at least 99%, or 100% identical to SEQ ID NO: 1700 and a VH at least 65%, at least 70%, at least 75%, at least 80%, at least 85%, at least 90%, at least 95%, at least 99%, or 100% identical to SEQ ID NO: 1704;
a VL at least 65%, at least 70%, at least 75%, at least 80%, at least 85%, at least 90%, at least 95%, at least 99%, or 100% identical to SEQ ID NO: 1711 and a VH at least 65%, at least 70%, at least 75%, at least 80%, at least 85%, at least 90%, at least 95%, at least 99%, or 100% identical to SEQ ID NO: 1714;
a VL at least 65%, at least 70%, at least 75%, at least 80%, at least 85%, at least 90%, at least 95%, at least 99%, or 100% identical to SEQ ID NO: 1721 and a VH at least 65%, at least 70%, at least 75%, at least 80%, at least 85%, at least 90%, at least 95%, at least 99%, or 100% identical to SEQ ID NO: 1722;
a VL at least 65%, at least 70%, at least 75%, at least 80%, at least 85%, at least 90%, at least 95%, at least 99%, or 100% identical to SEQ ID NO: 1724 and a VH at least 65%, at least 70%, at least 75%, at least 80%, at least 85%, at least 90%, at least 95%, at least 99%, or 100% identical to SEQ ID NO: 1726;
a VL at least 65%, at least 70%, at least 75%, at least 80%, at least 85%, at least 90%, at least 95%, at least 99%, or 100% identical to SEQ ID NO: 1730 and a VH at least 65%, at least 70%, at least 75%, at least 80%, at least 85%, at least 90%, at least 95%, at least 99%, or 100% identical to SEQ ID NO: 1734;
a VL at least 65%, at least 70%, at least 75%, at least 80%, at least 85%, at least 90%, at least 95%, at least 99%, or 100% identical to SEQ ID NO: 1740 and a VH at least 65%, at least 70%, at least 75%, at least 80%, at least 85%, at least 90%, at least 95%, at least 99%, or 100% identical to SEQ ID NO: 1742;
a VL at least 65%, at least 70%, at least 75%, at least 80%, at least 85%, at least 90%, at least 95%, at least 99%, or 100% identical to SEQ ID NO: 1746 and a VH at least 65%, at least 70%, at least 75%, at least 80%, at least 85%, at least 90%, at least 95%, at least 99%, or 100% identical to SEQ ID NO: 1750;
a VL at least 65%, at least 70%, at least 75%, at least 80%, at least 85%, at least 90%, at least 95%, at least 99%, or 100% identical to SEQ ID NO: 1755 and a VH at least 65%, at least 70%, at least 75%, at least 80%, at least 85%, at least 90%, at least 95%, at least 99%, or 100% identical to SEQ ID NO: 1759;
a VL at least 65%, at least 70%, at least 75%, at least 80%, at least 85%, at least 90%, at least 95%, at least 99%, or 100% identical to SEQ ID NO: 1755 and a VH at least 65%, at least 70%, at least 75%, at least 80%, at least 85%, at least 90%, at least 95%, at least 99%, or 100% identical to SEQ ID NO: 1760;
a VL at least 65%, at least 70%, at least 75%, at least 80%, at least 85%, at least 90%, at least 95%, at least 99%, or 100% identical to SEQ ID NO: 1730 and a VH at least 65%, at least 70%, at least 75%, at least 80%, at least 85%, at least 90%, at least 95%, at least 99%, or 100% identical to SEQ ID NO: 1734;
a VL at least 65%, at least 70%, at least 75%, at least 80%, at least 85%, at least 90%, at least 95%, at least 99%, or 100% identical to SEQ ID NO: 1764 and a VH at least 65%, at least 70%, at least 75%, at least 80%, at least 85%, at least 90%, at least 95%, at least 99%, or 100% identical to SEQ ID NO: 1765;
a VL at least 65%, at least 70%, at least 75%, at least 80%, at least 85%, at least 90%, at least 95%, at least 99%, or 100% identical to SEQ ID NO: 1766 and a VH at least 65%, at least 70%, at least 75%, at least 80%, at least 85%, at least 90%, at least 95%, at least 99%, or 100% identical to SEQ ID NO: 1768;
a VL at least 65%, at least 70%, at least 75%, at least 80%, at least 85%, at least 90%, at least 95%, at least 99%, or 100% identical to SEQ ID NO: 1771 and a VH at least 65%, at least 70%, at least 75%, at least 80%, at least 85%, at least 90%, at least 95%, at least 99%, or 100% identical to SEQ ID NO: 1775;
a VL at least 65%, at least 70%, at least 75%, at least 80%, at least 85%, at least 90%, at least 95%, at least 99%, or 100% identical to SEQ ID NO: 1779 and a VH at least 65%, at least 70%, at least 75%, at least 80%, at least 85%, at least 90%, at least 95%, at least 99%, or 100% identical to SEQ ID NO: 1783;
a VL at least 65%, at least 70%, at least 75%, at least 80%, at least 85%, at least 90%, at least 95%, at least 99%, or 100% identical to SEQ ID NO: 1784 and a VH at least 65%, at least 70%, at least 75%, at least 80%, at least 85%, at least 90%, at least 95%, at least 99%, or 100% identical to SEQ ID NO: 1785;
a VL at least 65%, at least 70%, at least 75%, at least 80%, at least 85%, at least 90%, at least 95%, at least 99%, or 100% identical to SEQ ID NO: 1788 and a VH at least 65%, at least 70%, at least 75%, at least 80%, at least 85%, at least 90%, at least 95%, at least 99%, or 100% identical to SEQ ID NO: 1792;
a VL at least 65%, at least 70%, at least 75%, at least 80%, at least 85%, at least 90%, at least 95%, at least 99%, or 100% identical to SEQ ID NO: 1794 and a VH at least 65%, at least 70%, at least 75%, at least 80%, at least 85%, at least 90%, at least 95%, at least 99%, or 100% identical to SEQ ID NO: 1797;
a VL at least 65%, at least 70%, at least 75%, at least 80%, at least 85%, at least 90%, at least 95%, at least 99%, or 100% identical to SEQ ID NO: 1800 and a VH at least 65%, at least 70%, at least 75%, at least 80%, at least 85%, at least 90%, at least 95%, at least 99%, or 100% identical to SEQ ID NO: 1804;
a VL at least 65%, at least 70%, at least 75%, at least 80%, at least 85%, at least 90%, at least 95%, at least 99%, or 100% identical to SEQ ID NO: 1806 and a VH at least 65%, at least 70%, at least 75%, at least 80%, at least 85%, at least 90%, at least 95%, at least 99%, or 100% identical to SEQ ID NO: 1809;
a VL at least 65%, at least 70%, at least 75%, at least 80%, at least 85%, at least 90%, at least 95%, at least 99%, or 100% identical to SEQ ID NO: 1812 and a VH at least 65%, at least 70%, at least 75%, at least 80%, at least 85%, at least 90%, at least 95%, at least 99%, or 100% identical to SEQ ID NO: 1816;
a VL at least 65%, at least 70%, at least 75%, at least 80%, at least 85%, at least 90%, at least 95%, at least 99%, or 100% identical to SEQ ID NO: 1819 and a VH at least 65%, at least 70%, at least 75%, at least 80%, at least 85%, at least 90%, at least 95%, at least 99%, or 100% identical to SEQ ID NO: 1823;
a VL at least 65%, at least 70%, at least 75%, at least 80%, at least 85%, at least 90%, at least 95%, at least 99%, or 100% identical to SEQ ID NO: 1826 and a VH at least 65%, at least 70%, at least 75%, at least 80%, at least 85%, at least 90%, at least 95%, at least 99%, or 100% identical to SEQ ID NO: 1830;
a VL at least 65%, at least 70%, at least 75%, at least 80%, at least 85%, at least 90%, at least 95%, at least 99%, or 100% identical to SEQ ID NO: 1980 and a VH at least 65%, at least 70%, at least 75%, at least 80%, at least 85%, at least 90%, at least 95%, at least 99%, or 100% identical to SEQ ID NO: 1984;
a VL at least 65%, at least 70%, at least 75%, at least 80%, at least 85%, at least 90%, at least 95%, at least 99%, or 100% identical to SEQ ID NO: 1988 and a VH at least 65%, at least 70%, at least 75%, at least 80%, at least 85%, at least 90%, at least 95%, at least 99%, or 100% identical to SEQ ID NO: 1992;
a VL at least 65%, at least 70%, at least 75%, at least 80%, at least 85%, at least 90%, at least 95%, at least 99%, or 100% identical to SEQ ID NO: 1996 and a VH at least 65%, at least 70%, at least 75%, at least 80%, at least 85%, at least 90%, at least 95%, at least 99%, or 100% identical to SEQ ID NO: 2000;
a VL at least 65%, at least 70%, at least 75%, at least 80%, at least 85%, at least 90%, at least 95%, at least 99%, or 100% identical to SEQ ID NO: 2004 and a VH at least 65%, at least 70%, at least 75%, at least 80%, at least 85%, at least 90%, at least 95%, at least 99%, or 100% identical to SEQ ID NO: 2008;
a VL at least 65%, at least 70%, at least 75%, at least 80%, at least 85%, at least 90%, at least 95%, at least 99%, or 100% identical to SEQ ID NO: 1833 and a VH at least 65%, at least 70%, at least 75%, at least 80%, at least 85%, at least 90%, at least 95%, at least 99%, or 100% identical to SEQ ID NO: 1837;
a VL at least 65%, at least 70%, at least 75%, at least 80%, at least 85%, at least 90%, at least 95%, at least 99%, or 100% identical to SEQ ID NO: 1840 and a VH at least 65%, at least 70%, at least 75%, at least 80%, at least 85%, at least 90%, at least 95%, at least 99%, or 100% identical to SEQ ID NO: 1844;
a VL at least 65%, at least 70%, at least 75%, at least 80%, at least 85%, at least 90%, at least 95%, at least 99%, or 100% identical to SEQ ID NO: 1847 and a VH at least 65%, at least 70%, at least 75%, at least 80%, at least 85%, at least 90%, at least 95%, at least 99%, or 100% identical to SEQ ID NO: 1851;
a VL at least 65%, at least 70%, at least 75%, at least 80%, at least 85%, at least 90%, at least 95%, at least 99%, or 100% identical to SEQ ID NO: 1854 and a VH at least 65%, at least 70%, at least 75%, at least 80%, at least 85%, at least 90%, at least 95%, at least 99%, or 100% identical to SEQ ID NO: 1858;
a VL at least 65%, at least 70%, at least 75%, at least 80%, at least 85%, at least 90%, at least 95%, at least 99%, or 100% identical to SEQ ID NO: 1861 and a VH at least 65%, at least 70%, at least 75%, at least 80%, at least 85%, at least 90%, at least 95%, at least 99%, or 100% identical to SEQ ID NO: 1865;
a VL at least 65%, at least 70%, at least 75%, at least 80%, at least 85%, at least 90%, at least 95%, at least 99%, or 100% identical to SEQ ID NO: 1361 and a VH at least 65%, at least 70%, at least 75%, at least 80%, at least 85%, at least 90%, at least 95%, at least 99%, or 100% identical to SEQ ID NO: 1365;
a VL at least 65%, at least 70%, at least 75%, at least 80%, at least 85%, at least 90%, at least 95%, at least 99%, or 100% identical to SEQ ID NO: 1868 and a VH at least 65%, at least 70%, at least 75%, at least 80%, at least 85%, at least 90%, at least 95%, at least 99%, or 100% identical to SEQ ID NO: 1871;
a VL at least 65%, at least 70%, at least 75%, at least 80%, at least 85%, at least 90%, at least 95%, at least 99%, or 100% identical to SEQ ID NO: 1874 and a VH at least 65%, at least 70%, at least 75%, at least 80%, at least 85%, at least 90%, at least 95%, at least 99%, or 100% identical to SEQ ID NO: 1877;
a VL at least 65%, at least 70%, at least 75%, at least 80%, at least 85%, at least 90%, at least 95%, at least 99%, or 100% identical to SEQ ID NO: 1880 and a VH at least 65%, at least 70%, at least 75%, at least 80%, at least 85%, at least 90%, at least 95%, at least 99%, or 100% identical to SEQ ID NO: 1884;
a VL at least 65%, at least 70%, at least 75%, at least 80%, at least 85%, at least 90%, at least 95%, at least 99%, or 100% identical to SEQ ID NO: 1887 and a VH at least 65%, at least 70%, at least 75%, at least 80%, at least 85%, at least 90%, at least 95%, at least 99%, or 100% identical to SEQ ID NO: 1890;
a VL at least 65%, at least 70%, at least 75%, at least 80%, at least 85%, at least 90%, at least 95%, at least 99%, or 100% identical to SEQ ID NO: 1893 and a VH at least 65%, at least 70%, at least 75%, at least 80%, at least 85%, at least 90%, at least 95%, at least 99%, or 100% identical to SEQ ID NO: 1897;
a VL at least 65%, at least 70%, at least 75%, at least 80%, at least 85%, at least 90%, at least 95%, at least 99%, or 100% identical to SEQ ID NO: 1900 and a VH at least 65%, at least 70%, at least 75%, at least 80%, at least 85%, at least 90%, at least 95%, at least 99%, or 100% identical to SEQ ID NO: 1904;
a VL at least 65%, at least 70%, at least 75%, at least 80%, at least 85%, at least 90%, at least 95%, at least 99%, or 100% identical to SEQ ID NO: 1907 and a VH at least 65%, at least 70%, at least 75%, at least 80%, at least 85%, at least 90%, at least 95%, at least 99%, or 100% identical to SEQ ID NO: 1910;
a VL at least 65%, at least 70%, at least 75%, at least 80%, at least 85%, at least 90%, at least 95%, at least 99%, or 100% identical to SEQ ID NO: 1913 and a VH at least 65%, at least 70%, at least 75%, at least 80%, at least 85%, at least 90%, at least 95%, at least 99%, or 100% identical to SEQ ID NO: 1916;
a VL at least 65%, at least 70%, at least 75%, at least 80%, at least 85%, at least 90%, at least 95%, at least 99%, or 100% identical to SEQ ID NO: 1919 and a VH at least 65%, at least 70%, at least 75%, at least 80%, at least 85%, at least 90%, at least 95%, at least 99%, or 100% identical to SEQ ID NO: 1923;
a VL at least 65%, at least 70%, at least 75%, at least 80%, at least 85%, at least 90%, at least 95%, at least 99%, or 100% identical to SEQ ID NO: 1926 and a VH at least 65%, at least 70%, at least 75%, at least 80%, at least 85%, at least 90%, at least 95%, at least 99%, or 100% identical to SEQ ID NO: 1930;
a VL at least 65%, at least 70%, at least 75%, at least 80%, at least 85%, at least 90%, at least 95%, at least 99%, or 100% identical to SEQ ID NO: 1933 and a VH at least 65%, at least 70%, at least 75%, at least 80%, at least 85%, at least 90%, at least 95%, at least 99%, or 100% identical to SEQ ID NO: 1937;
a VL at least 65%, at least 70%, at least 75%, at least 80%, at least 85%, at least 90%, at least 95%, at least 99%, or 100% identical to SEQ ID NO: 1940 and a VH at least 65%, at least 70%, at least 75%, at least 80%, at least 85%, at least 90%, at least 95%, at least 99%, or 100% identical to SEQ ID NO: 1944;
a VL at least 65%, at least 70%, at least 75%, at least 80%, at least 85%, at least 90%, at least 95%, at least 99%, or 100% identical to SEQ ID NO: 1947 and a VH at least 65%, at least 70%, at least 75%, at least 80%, at least 85%, at least 90%, at least 95%, at least 99%, or 100% identical to SEQ ID NO: 1950;
a VL at least 65%, at least 70%, at least 75%, at least 80%, at least 85%, at least 90%, at least 95%, at least 99%, or 100% identical to SEQ ID NO: 1952 and a VH at least 65%, at least 70%, at least 75%, at least 80%, at least 85%, at least 90%, at least 95%, at least 99%, or 100% identical to SEQ ID NO: 1956;
a VL at least 65%, at least 70%, at least 75%, at least 80%, at least 85%, at least 90%, at least 95%, at least 99%, or 100% identical to SEQ ID NO: 1958 and a VH at least 65%, at least 70%, at least 75%, at least 80%, at least 85%, at least 90%, at least 95%, at least 99%, or 100% identical to SEQ ID NO: 1962;
a VL at least 65%, at least 70%, at least 75%, at least 80%, at least 85%, at least 90%, at least 95%, at least 99%, or 100% identical to SEQ ID NO: 1965 and a VH at least 65%, at least 70%, at least 75%, at least 80%, at least 85%, at least 90%, at least 95%, at least 99%, or 100% identical to SEQ ID NO: 1969; or
a VL at least 65%, at least 70%, at least 75%, at least 80%, at least 85%, at least 90%, at least 95%, at least 99%, or 100% identical to SEQ ID NO: 1971 and a VH at least 65%, at least 70%, at least 75%, at least 80%, at least 85%, at least 90%, at least 95%, at least 99%, or 100% identical to SEQ ID NO: 1974.

In some aspects, any one of the antigen binding polypeptides, antigen binding polypeptide complexes, polypeptides, antibodies or antigen binding fragments thereof disclosed herein have a poly-reactivity score of from 0) to about 100, from 0 to about 90, from 0 to about 80, from 0 to about 70, from 0) to about 60, from 0) to about 50, from 0 to about 40, from 0 to about 30, from 0 to about 25, from 0) to about 20, from 0 to about 15, from 0 to about 10, or from 0 to about 5.

In some aspects, any one of the antigen binding polypeptides, antigen binding polypeptide complexes, polypeptides, antibodies or antigen binding fragments thereof disclosed herein have a poly-reactivity score of from 0 to about 50, from 0 to about 40, from 0 to about 30, from 0 to about 25, from 0 to about 20, from 0 to about 15, from 0 to about 10, or from 0 to about 5.

In some aspects, any one of the antigen binding polypeptides, antigen binding polypeptide complexes, polypeptides, antibodies or antigen binding fragments thereof disclosed herein have a poly-reactivity score of from 0 to about 10, or from 0 to about 5.

In some aspects, any one of the antigen binding polypeptides, antigen binding polypeptide complexes, polypeptides, antibodies or antigen binding fragments thereof disclosed herein have a poly-reactivity score of about 100, 95, 90, 85, 80, 75, 70, 65, 60, 55, 50, 45, 40, 35, 30, 25, 20, 19, 18, 17, 16, 15, 14, 13, 12, 10, 9, 8, 7, 6, 5, 4, 3, 2, 1, or 0.

In some aspects, any one of the antigen binding polypeptides, antigen binding polypeptide complexes, polypeptides, antibodies or antigen binding fragments thereof disclosed herein have a poly-reactivity score of about 20, 19, 18, 17, 16, 15, 14, 13, 12, 10, 9, 8, 7, 6, 5, 4, 3, 2, 1, or 0.

In some aspects, any one of the antigen binding polypeptides, antigen binding polypeptide complexes, polypeptides, antibodies or antigen binding fragments thereof disclosed herein have a poly-reactivity score of about 10, 9, 8, 7, 6, 5, 4, 3, 2, 1, or 0.

### Amino Acid Linkers

In some aspects, an antigen binding polypeptide or polypeptide complex (e.g., an antibody or antigen binding fragment thereof) provided herein comprises one or more optional amino acid linkers between two or more regions of the antigen binding polypeptide or polypeptide complex.

As used herein, an “amino acid linker” can be a single amino acid or short amino acid sequence that is capable of joining two regions of an antigen binding polypeptide or polypeptide complex provided herein in a stable manner that maintains or promotes a function associated with the regions. However, as used herein, an “amino acid linker” can also include where an amino acid linker is not present between two regions (referred to herein as an amino acid linker having 0 amino acids). In some aspects, an amino acid linker is represented herein in a structure of an antigen binding polypeptide or polypeptide complex by the abbreviation “1” or “L” and a number (e.g., L1 to denote a first linker. L2 to denote a second linker. L3 to denote a third linker. L4 to denote a fourth linker. L5 to denote a fifth linker. L6 to denote a sixth linker. L7 to denote a seventh linker, and L8 to denote an eighth linker. L9 to denote a ninth linker. L10) to denote a tenth linker). In some aspects, such enumerated amino acid linkers (e.g., L1) can have the same or different sequence as any other enumerated amino acid linker (e.g., L2, etc.).

In one aspect, an amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) has a length of from 0 amino acids (i.e., an amino acid linker is not present) to about 50 amino acids. In another aspect, the amino acid linker has a length of from 0 amino acids to about 45 amino acids. 0 amino acids to about 40 amino acids. 0 amino acids to about 35 amino acids. 0 amino acids to about 30 amino acids. 0 amino acids to about 25 amino acids. 0 amino acids to about 20 amino acids. 0 amino acids to about 15 amino acids. 0 amino acids to about 10 amino acids. 0 amino acids to about 5 amino acids, about 1 amino acid to about 45 amino acids, about 1 amino acid to about 40) amino acids, about 1 amino acid to about 35 amino acids, about 1 amino acid to about 30 amino acids, about 1 amino acid to about 25 amino acids, about 1 amino acid to about 20 amino acids. 1 amino acid to about 15 amino acids, about 1 amino acid to about 10 amino acids, about 1 amino acid to about 5 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10) amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20) amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30) amino acids to about 45 amino acids, about 30 amino acids to about 40 amino acids, about 30 amino acids to about 35 amino acids, about 40 amino acids to about 50 amino acids, about 40 amino acids to about 45 amino acids, or about 45 amino acids to about 50 amino acids

In another aspect, the amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) has 0 amino acids (i.e., an amino acid linker is not present) or about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40), about 45, or about 50 amino acids.

In one aspect, the amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) consists of one or more amino acid residues. In one aspect, the amino acid residues are selected from the group consisting of glycine, alanine, serine, threonine, cysteine, asparagine, glutamine, leucine, isoleucine, valine, proline, histidine, aspartic acid, glutamic acid, lysine, arginine, methionine, phenylalanine, tryptophan, and tyrosine.

In one aspect, an amino acid linker is non-immunogenic. In one aspect, a non-immunogenic linker consists of serine, glycine and/or alanine residues, or consists of serine and/or glycine residues. In another aspect, an amino acid linker does not contain a T cell epitope or consensus T cell epitope.

In one aspect, an amino acid linker consists of one or more residues of alanine, cysteine, glycine, isoleucine, leucine, methionine, phenylalanine, proline, tryptophan, tyrosine, or valine (e.g., one or more of L1, L2, L3, L4, L5, L6, L7, L8, L9, or L10 of a first, second, third and/or fourth polypeptide of an antigen binding polypeptide or polypeptide complex structure provided herein).

Amino acid linker sequences that can be used with the antigen binding polypeptides or polypeptide complexes (e.g., an antibody or antigen binding fragment thereof) provided herein are well known and can be incorporated into the antigen binding polypeptides and polypeptide complexes provided herein using routine molecular biology and recombinant DNA techniques. Sec, e.g., Chen et al., Adv Drug Deliv Rev., 65(10):1357-1369, 2013; and Chichili et al., Protein Sci., 22(2):153-167, 2013.

In one aspect, an amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In one aspect, an amino acid linker (e.g., one or more of L1, L2, L3, L4, L5, L6, L7, L8, L9, or L10 of a first, second, third and/or fourth polypeptide of an antigen binding polypeptide or polypeptide complex structure provided herein) comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971.

In some aspects, L1 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects, L1 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects, L1 comprises 0 amino acids (i.e., L1 is not present).

In some aspects. L2 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L2 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L2 comprises 0 amino acids (i.e., L2 is not present).

In some aspects. L3 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L3 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L3 comprises 0 amino acids (i.e., L3 is not present).

In some aspects. L4 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L4 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L4 comprises 0 amino acids (i.e., L4 is not present).

In some aspects. L5 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L5 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L5 comprises 0 amino acids (i.e., L5 is not present).

In some aspects. L6 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L6 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L6 comprises 0 amino acids (i.e., L6 is not present).

In some aspects. L7 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L7 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L7 comprises 0 amino acids (i.e., L7 is not present).

In some aspects. L8 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L8 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L8 comprises 0 amino acids (i.e., L8 is not present).

In some aspects. L9 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L9 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L9 comprises 0 amino acids (i.e., L9 is not present).

In some aspects. L10 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L10 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L10 comprises 0 amino acids (i.e., L10 is not present).

In some aspects. L11 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L10 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L10 comprises 0 amino acids (i.e., L11 is not present).

In some aspects. L12 comprises an amino acid sequence of G, or A, or an amino acid sequence having at least 65%, at least 70%, at least 75%, at least 80%, at least 85%, at least 90%, at least 95%, at least 99%, or 100% identity to amino acid sequence GSS or ASG, or an amino acid sequence having at least 65%, at least 70%, at least 75%, at least 80%, at least 85%, at least 90%, at least 95%, at least 99%, or 100% identity to any one of SEQ ID NOs: 333-373 or 967-971. In some aspects. L10 comprises an amino acid sequence having at least 65%, at least 70%, at least 75%, at least 80%, at least 85%, at least 90%, at least 95%, at least 99%, or 100% identity to any one of SEQ ID NOs: 333-345 or 967-971. In some aspects. L10 comprises 0 amino acids (i.e., L12 is not present).

### Modifications

In some aspects, an antigen binding polypeptide or polypeptide complex (e.g., an antibody or antigen binding fragment thereof) provided herein comprises an effector function mutation or half-life extension mutation.

Effector functions are an important part of the humoral immune response and form a link between innate and adaptive immunity. Most effector functions are induced via the Fc region of an antibody, which can interact with complement proteins and specialized Fc receptors. As used herein, an “effector function mutation” or “Fc effector function mutation” refer to a change in the amino acid sequence, typically in the Fc region, which can increase or decrease effector function, for example, increase binding affinity of Fc for specific Fc receptors, or increase antibody-dependent cellular cytotoxicity (ADCC) activity.

“Half-life” of a pharmaceutically active substance is the time it takes for the amount of the substance, once administered to the body, to reduce by half. A “half-life extension mutation” of an antigen binding polypeptide or polypeptide complex provided herein refers to a change in the amino acid sequence, typically in the Fc region, which increases the half-life of the antigen binding polypeptide or polypeptide complex (e.g., by increasing Fc receptor binding affinity, slowing off-rate for Fc and Fc receptors, and/or increased sialylation).

Examples of Fc effector function mutations that decrease or knockout function include, but are not limited to, the following substitutions in the Fc region, based on the EU numbering scheme; (LA) L234A, L235A, (LS) M428L, N434S, P239A, N297A, or equivalent thereof, or a combination thereof.

Examples of Fc effector function mutations that increase function include, but are not limited to, the following substitutions in the Fc region, based on the EU numbering scheme: S298A/E333A/K334A, S239D/1332E, S239D/A330L/1332E, G236A/S239D/1332E, or equivalent thereof, or a combination thereof.

Additional examples of effector function mutations, half-life extension mutations and methods for incorporating the same into an amino acid sequence are known and described, for example, in Saunders. “Conceptual Approaches to Modulating Antibody Effector Functions and Circulation Half-Life.” Front. Immunol. Jun. 7, 2019.

In another aspect, an antigen binding polypeptide or polypeptide complex (e.g., an antibody or antigen binding fragment thereof) provided herein comprises one or more knob-into-hole modifications.

The term “knob-into-hole modification.” as used herein, refers to a genetic modification that directs the pairing of two polypeptides to promote heterodimerization. In one aspect, the modification introduces a protuberance (knob) into one polypeptide and a cavity (hole) into the other polypeptide at an interface in which the two polypeptides interact. In another aspect, a knob-into-hole modification can be created by introducing only a hole modification, for example, by replacing an amino acid residue with a smaller side chain than the original amino acid residue (e.g., a substitution of one or more serine, threonine, valine or alanine residues, or a combination thereof). In yet another aspect, a knob-into-hole modification can be created by introducing only a knob modification, for example, by replacing an amino acid residue with a larger side chain than the original amino acid residue (e.g., a substitution of one or more tryptophan or tyrosine residues, or a combination thereof).

In one aspect, the knob-into-hole modification is in the binding interface of two Fc regions, the binding interface of two CH2 regions, the binding interface of two CH3 regions, the binding interface of a CL region and a CH1 region, or the binding interface of a VH region and a VL region. Sec, e.g., U.S. Pub. No. 2007/0178552, Int'l Pub. No, WO 96/027011, Int'l Pub. No, WO 98/050431 and Zhu et al., Protein Science 6:781-788, 1987.

In one aspect, the antigen binding polypeptide or polypeptide complex comprises one, two, three, four, five, six, seven, eight, nine, ten, or more knob-into-hole modifications.

Knob-into-hole modifications are well known and can be incorporated into antigen binding polypeptides and polypeptide complexes provided herein using routine molecular biology and recombinant DNA techniques. See, e.g., U.S. Pub. No. 2003/0078385; Int'l Pub. No, WO 96/027011; Ridgway et al., Protein Eng., 9:617-621, 1996; and Merchant et al., Nat. Biotechnol., 16:677-681, 1998.

In one aspect, the knob-into-hole modification is an amino acid substitution. As used herein, such a substitution is described based on the EU numbering scheme of Kabat, which corresponds to the numbering in the Protein Data Bank (PDB).

In one aspect, the knob-into-hole modification is a knob substitution of S354C, T366W, or equivalent thereof, or a combination thereof based on the EU numbering scheme.

In one aspect, the knob-into-hole modification is a hole substitution of Y349C, T366S, L368A, Y407V, or equivalent thereof, or a combination thereof, based on the EU numbering scheme.

In another aspect, the knob-into-hole modifications are knob substitutions of S354C, T366W, or equivalent thereof, based on the EU numbering scheme.

In another aspect, the knob-into-hole modifications are hole substitutions of Y349C, T366S, L368A and Y407V, or equivalent thereof, based on the EU numbering scheme.

In another aspect, an antigen binding polypeptide complex is an IgG1 or IgG4 antibody or antigen binding fragment thereof and the knob-into-hole modifications are knob substitutions of S354C and T366W and/or hole substitutions of Y349C, T366S, L368A and Y407V, or equivalent thereof.

In one aspect, the antigen binding polypeptide complex is an IgG1 or IgG4 antibody or antigen binding fragment thereof and the knob-into-hole modifications are knob substitutions of S354C and T366W, or equivalent thereof.

In one aspect, the antigen binding polypeptide complex is an IgG1 or IgG4 antibody or antigen binding fragment thereof and the knob-into-hole modifications are hole substitutions of Y349C, T366S, L368A and Y407V, or equivalent thereof.

In one aspect, the antigen binding polypeptide complex is an IgG1 or IgG4 antibody or antigen binding fragment thereof and the knob-into-hole modifications are knob substitutions of S354C and T366W and hole substitutions of Y349C, T366S, L368A and Y407V, or equivalent thereof.

In one aspect, the antigen binding polypeptide complex is an IgG1 or IgG4 antibody or antigen binding fragment thereof and the Fc region comprises a knob-into-hole modification comprising knob substitutions of S354C and T366W, or equivalent thereof, and/or hole substitutions of Y349C, T366S, L368A and Y407V, or equivalent thereof, and/or a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution, L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In one aspect, the antigen binding polypeptide complex is an IgG1 or IgG4 antibody or antigen binding fragment thereof and the Fc region comprises knob substitutions of S354C and T366W, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

In one aspect, the antigen binding polypeptide complex is an IgG1 or IgG4 antibody or antigen binding fragment thereof and the Fc region comprises hole substitutions of Y349C, T366S, L368A and Y407V, or equivalent thereof, and a half-life extension amino acid substitution comprising: a LA (M428L and N434A, or equivalent thereof, based on the EU numbering scheme) amino acid substitution, a LS (M428L and N434S, or equivalent thereof, based on the EU numbering scheme) amino acid substitution. L234F and L235E, or equivalent thereof (based on the EU numbering scheme) amino acid substitutions, a TM (L234F, L235E, and P331S, or equivalent thereof, based on the EU numbering scheme) amino acid substitution, a YTE (M252Y, S254T, and T256E, or equivalent thereof, based on the EU numbering scheme) amino acid substitution, or any combination thereof.

### Detectable Labels

In some aspects, an antigen binding polypeptide or polypeptide complex (e.g., an antibody or antigen binding fragment thereof) provided herein comprises one or more detectable labels. An antigen binding polypeptide or polypeptide complex (e.g., an antibody or antigen binding fragment thereof) containing a detectable label is useful in therapeutic, diagnostic, imaging (e.g., radioimaging), or basic research applications.

In one aspect, the detectable label is a radioactive label. Examples of a radioactive label include, but are not limited to, the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc, ¹⁸⁶Re and ⁸⁹Zr.

In another aspect, the detectable label is a chemiluminescent label, fluorescent label, enzyme, biotin, or a combination thereof.

In another aspect, the detectable label is a peptide tag. In one aspect, the peptide tag is located at or near the N-terminus of the polypeptide or polypeptide complex. In another aspect, the peptide tag is located or near at the C-terminus of the polypeptide or polypeptide complex. In another aspect, the peptide tag is an affinity tag or fusion tag.

In another aspect, the detectable label is a polyhistidine tag, polyarginine tag, glutathione-S-transferase (GST), maltose binding protein (MBP), chitin binding protein (CBP), Strep-tag, thioredoxin (TRX), poly (NANP), FLAG tag, ALFA-tag, V5-tag, Myc-tag, hemagglutinin (HA) tag, Spot tag, T7 tag, NE tag, or green fluorescence protein (GFP), or a combination thereof. In one aspect, the polyhistidine tag consists of from about 4 to about 10 histidine residues. In one aspect, the polyhistidine tag consists of about 4, about 5, about 6, about 7, about 8, about 9, or about 10 histidine residues.

Additional examples of detectable labels and methods for introducing detectable labels into a polypeptide are known and include routine chemical, molecular biology and recombinant DNA techniques. See, e.g., Hnatowich et al., Science. 220 (4597): 613-615, 1983; Yao et al., Int. J. Mol. Sci., 17 (2): 194, 2016; Kimple et al., Curr. Protoc. Protein Sci., 73; Unit 9.9, 2013; Sambrook J. Fritsch E F. Molecular Cloning: A Laboratory Manual. Cold Spring Harbor Laboratory Press: Cold Spring Harbor, N.Y.: 1989; Molecular Cell Biology. 4^{th} edition. Section 3.5. Purifying. Detecting and Characterizing Proteins; and Mahmoodi et al., Cogent Biology. 5 (1):DOI: 10/1080/23312025.2019.1665406.

### Polypeptides, Polynucleotides, Vectors, Cells, and Protein Production Methods

In some aspects, a polynucleotide encoding an antigen binding polypeptide or antigen polypeptide complex (e.g., an antibody or antigen binding fragment thereof) is provided herein.

In some aspects, the polypeptides provided herein comprise a CL and a CH1. In some aspects, a polypeptide provided herein has an improved isoelectric point compared to a same polypeptide not comprising a CL and a CH1. It is to be understood that “a same polypeptide not comprising a CL and a CH1” refers to a polypeptide having a same amino acid sequence as the reference polypeptide exception made for the CL and the CH1 being absent from the “same polypeptide not comprising a CL and a CH1.” For example, a reference polypeptide (polypeptide R) may comprise an amino acid sequence represented by the formula VL1-CL-VL2-VH2-VH1-CH1. “a same polypeptide not comprising a CL and a CH1” (polypeptide R′) it is to be understood as comprising an amino acid sequence that is represented by the formula VL1-VL2-VH2-VH1, wherein the VL1, the VL2, the VH1, and the VH1 in the polypeptide R comprise an amino acid sequence identical to the amino acid sequence comprised in the VL1, the VL2, the VH1, and the VH1 comprised in the polypeptide R′.

Without wishing to be bound to any particular theory, it is believed that the isoelectric point (pI) corresponds to the pH at which the antibody has no electrical charge. If the pH of the surrounding environment is below the antibody's pI, then the antibody molecule carries a net positive charge. If the pH of the surrounding environment is above the antibody's pI, then the antibody molecule carries a net negative charge. The pI of an antibody can influence its pharmacokinetics properties. The surface of most cells is negatively charged, thus antibodies (or antigen binding fragments thereof) having a net positive charge may possess advantageous pharmacokinetics properties. Increased net positive charge can also result in increased blood clearance and increased tissue retention with shorter half-life, whereas increased net negative charge can results in decreased tissue uptake and longer half-life. For an antibody to be positively charged the environmental pH needs to be below the pI of the antibody, and for an antibody to be negatively charged the environmental pH needs to be above the pI of the antibody. Physiological pH is of about 7-7.5 (generally between 7.35 and 7.45).

In other aspects, provided herein is a vector comprising a polynucleotide provided herein.

In yet other aspects, provided herein is a host cell comprising a polynucleotide or vector provided herein.

As used herein, the term “host cell” can be any type of cell, e.g., a primary cell, a cell in culture, a cell from a cell line, or a cell comprised in an organism. In some aspects, the term “host cell” refers to a cell containing a foreign nucleic acid molecule (e.g., a cell transformed, transfected, or transduced with a polynucleotide (e.g., DNA or mRNA)) as well as the progeny or potential progeny of such a cell. Progeny of such a cell may not be identical to the parent cell, e.g., due to mutations or environmental influences that may occur in succeeding generations or integration of the nucleic acid molecule into the host cell genome. Methods which are well known to those skilled in the art can be used to construct vectors encoding antigen binding polypeptides and polypeptide complexes (e.g., CDR, VH, VL, heavy chain and/or light chain coding sequences and appropriate transcriptional and translational control signals). These methods include, for example, in vitro recombinant DNA techniques, synthetic techniques, and in vivo genetic engineering (e.g., transgenesis).

A vector can be transformed, transferred, or transduced into a host cell by conventional techniques and the resulting cell can then be cultured by conventional techniques to produce an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain). Thus, provided herein are host cells containing a polynucleotide encoding an antigen binding polypeptide or polypeptide complex comprising, e.g., comprising six CDRs, VH, VL, VH and VL, heavy chain, light chain, or heavy and light chain, or a domain thereof (e.g., one or more CDRs, VH, VL, VH and VL, heavy chain, or light chain), operably linked to a promoter for expression of such sequences in the host cell.

In some aspects, vectors encoding both heavy and light chains, or a domain thereof, individually, can be co-expressed in the host cell for expression. In some aspects, a host cell contains a vector comprising a polynucleotide encoding both a heavy chain and light chain, or a domain thereof. In some aspects, a host cell contains two different vectors, a first vector comprising a polynucleotide encoding a heavy chain or a domain thereof, and a second vector comprising a polynucleotide encoding a light chain or a domain thereof. In some aspects, a first host cell comprises a first vector comprising a polynucleotide encoding a heavy chain or a domain thereof, and a second host cell comprises a second vector comprising a polynucleotide encoding a light chain or a domain thereof. In some aspects, provided herein is a population of host cells comprising such a first host cell and such a second host cell.

In some aspects, provided herein is a population of vectors comprising a first vector comprising a polynucleotide encoding a light chain or domain thereof, and a second vector comprising a polynucleotide encoding a heavy chain or domain thereof. Alternatively, a single vector can be used which encodes, and is capable of expressing, both heavy and light chain polypeptides or a domain thereof.

A variety of host-vector systems can be utilized to express the polypeptides and polypeptide complexes provided herein. Such host-vector systems represent vehicles by which the coding sequences of interest can be produced and subsequently purified, but also represent cells which can, when transformed or transfected with the appropriate nucleotide coding sequences, express a polypeptide or polypeptide complex provided herein in situ. These include but are not limited to microorganisms such as bacteria (e.g., E. coli and B. subtilis) transformed with recombinant bacteriophage DNA, plasmid DNA or cosmid DNA expression vectors containing antibody or antigen binding fragment thereof coding sequences; yeast (e.g., Saccharomyces pichia) transformed with recombinant yeast expression vectors containing antibody or antigen binding fragment thereof coding sequences; insect cell systems infected with recombinant virus expression vectors (e.g., baculovirus) containing antibody or antigen binding fragment thereof coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody or antigen binding fragment thereof coding sequences; or mammalian cell systems (e.g., COS (e.g., COSI or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40), YB/20, and BMT10 cells) harboring recombinant expression constructs containing promoters derived from the genome of mammalian cells (e.g., metallothionein promoter) or from mammalian viruses (e.g., the adenovirus late promoter: the vaccinia virus 7.5K promoter). In some aspects, cells for expressing polypeptide or polypeptide complexes provided herein are CHO cells, for example CHO cells from the CHO GS System™ (Lonza). In some aspects, cells for expressing the polypeptides or polypeptide complexes provided herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells) are used for the expression of recombinant polypeptides. For example, mammalian cells such as Chinese hamster ovary (CHO) cells in conjunction with a vector such as the major intermediate early gene promoter element from human cytomegalovirus is an effective expression system for polypeptides (Foecking M K & Hofstetter H (1986) Gene 45:101-105; and Cockett M I et al., (1990) Biotechnology 8:662-667). In some aspects, the polypeptides or polypeptide complexes provided herein are produced by HEK-293T cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK. Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COSI or COS), PER.C6, VERO. HsS78Bst, HEK-293T, HepG2, SP210, R1.1, B-W. L-M, BSC1, BSC40), YB/20, BMT10) and HsS78Bst cells.

Once a polypeptide or polypeptide complex provided herein has been produced by recombinant expression, it can be purified by any method known in the art for purification of a protein or immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the polypeptides or polypeptide complexes provided herein can be fused to heterologous polypeptide sequences provided herein (e.g., peptide tags) or otherwise known in the art to facilitate purification.

In some aspects, a polypeptide or polypeptide complex provided herein is isolated or purified. Generally, an isolated polypeptide or polypeptide complex is one that is substantially free of other polypeptides or polypeptide complexes with different antigenic specificities. For example, in some aspects, a preparation of a polypeptide or polypeptide complex provided herein is substantially free of cellular material and/or chemical precursors.

A vector can be transformed, transferred, or transduced into a host cell comprised in an organism by conventional techniques. The resulting host cell comprised in an organism can produce an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain).

Additionally, the resulting host cell comprised in an organism, as well as a host cell not comprised in an organism (e.g., a cell maintained in culture in vitro), can transmit copies of the nucleotide sequence comprised in the vector to its progeny. A nucleic acid sequence comprised in the vector can integrate in the genome of the host cell, replicate with the genome of the host cell, and thus be transmitted to the progeny of the host cell. A nucleic acid sequence comprised in the vector can also not integrate into the genome of the host cell, but instead can be maintained extrachromosomally in the host cell. In this case the vector may further comprise sequence elements that allow the nucleotide sequence comprised in the vector to self replicate. Self-replicated copies of the nucleotide sequence comprised in the vector can then be transmitted to the progeny of the host cell.

A vector disclosed herein can be a DNA vector, an RNA vector (e.g., mRNA), or a combination thereof, DNA and/or RNA vectors can be introduced into isolated host cells (e.g., cell in culture in vitro) or into host cells comprised in an organism.

In some aspects, the vectors provided herein are RNA vectors (e.g., mRNA vectors).

In some aspects, the present invention comprises a nucleic acid encoding an antigen binding polypeptide or antigen binding polypeptide complex for in vivo administration. In some aspects, the nucleic acid encoding an antigen binding polypeptide or antigen binding polypeptide complex is a stabilized nucleic acid selected from a stabilized mRNA.

In some aspects, the present disclosure relates to one or more mRNA that encodes an antigen binding polypeptide or antigen binding polypeptide complex (e.g., antibody or antigen binding fragment thereof).

In some aspects, a nucleic acid encoding an antigen binding polypeptide or antigen binding polypeptide complex comprises said nucleic acid or mRNA and a carrier to form a pharmaceutical composition.

The term “mRNA.” as used herein, refers to a single stranded RNA that encodes the amino acid sequence of one or more polypeptides (e.g., an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain)). The term “mRNA.” as used herein includes in vitro transcribed RNA (IVT RNA) or synthetic RNA. An mRNA molecule may also contain a 5′ untranslated region (5′-UTR), and/or a 3′ untranslated region (3′-UTR). In some aspects, the RNA is produced by in vitro transcription or chemical synthesis. In one aspect, the mRNA is produced by in vitro transcription using a DNA template where DNA refers to a nucleic acid that contains deoxyribonucleotides.

As used herein the term “transcription” relates to a process, wherein the genetic code in a DNA sequence is transcribed into RNA. Subsequently, the RNA may be translated into peptide or protein. According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts, and/or isolated nucleotides, enzymes, co-enzymes, and co-factors in the appropriate reaction conditions (e.g., controlled temperature, controlled pH). Preferably, cloning vectors are applied for the generation of transcripts. These cloning vectors are generally designated as transcription vectors and are according to the present invention encompassed by the term “vector”. According to the present invention, the mRNA used in the present invention preferably is in vitro transcribed mRNA (IVT-mRNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid (e.g., cDNA) and introducing it into an appropriate vector for in vitro transcription. The nucleic acid cloned and inserted into the appropriate vector may be obtained by reverse transcription of RNA, directly isolated from the source organism, amplified by PCR, synthesized in vitro, or any combination thereof, or obtained and/or manipulated by any means known in the art. In certain aspects of the present disclosure, the RNA is “replicon RNA” or simply a “replicon”, in particular “self-replicating RNA” or “self-amplifying RNA”.

In some aspects, the mRNAs provided herein contain structural elements optimized for maximal efficacy of the mRNA with respect to stability and translational efficiency (5′-cap. 5′-UTR. 3′-UTR, poly(A) sequence). In some aspects, the messenger RNA (mRNA) molecule provided herein further comprises (i) at least one 5′ untranslated region (5′-UTR), and/or (ii) at least one 3′ untranslated region (3′-UTR), operably linked to the open reading frame (ORF). As used herein, the term “5′ untranslated region”, or “5′ UTR” refers to a region of a messenger RNA (mRNA) that is directly upstream from the initiation codon, and it is important for the regulation of translation of a transcript. The 5′ UTR may comprise a 5′-cap. In some aspects, the mRNA according to the present disclosure comprises a 5′-cap. In some aspects, the mRNA of the present disclosure does not have uncapped 5′-triphosphates. In some aspects, the mRNA may be modified by a 5′-cap analog. The term “5′-cap” refers to a structure found on 5′-end of an mRNA molecule and generally consists of a guanosine nucleotide connected to the mRNA via a 5′- to 5′-triphosphate linkage. In one aspect, this guanosine is methylated at the 7-position. Providing an mRNA with a 5′-cap or 5′-cap analog may be achieved by in vitro transcription, in which 5′-cap is co-transcriptionally expressed into the mRNA strand, or may be attached to mRNA post-transcriptionally using capping enzymes. As used herein, the term “3′ untranslated region”, or “3′-UTR”, refers to a region of messenger RNA (mRNA) that immediately follows the translation termination codon. The 3′-UTR often contains regulatory regions that post-transcriptionally influence gene expression. Regulatory regions within the 3′-untranslated region can influence polyadenylation, translation efficiency, localization, and stability of the mRNA. The 3′ UTR may comprise 3′ poly(A) tail. As used herein, the term “3′ poly(A) tail”, or “poly(A) sequence”, or “poly-A tail” refers to an uninterrupted or interrupted sequence of adenylate residues which is typically located at 3′-end of an mRNA molecule. Poly(A) sequences are known to those of skill in the art and may follow 3′-UTR in the mRNAs described herein. An uninterrupted poly(A) sequence is characterized by consecutive adenylate residues. In nature, an uninterrupted poly(A) sequence is typical, mRNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

In some aspects, the invention features messenger RNA (mRNA) molecule comprising an open reading frame (ORF) of (i.e., encoding) an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain).

### Pharmaceutical Compositions and Kits

In some aspects, provided herein is a pharmaceutical composition comprising an antigen binding polypeptide, antigen binding polypeptide complex (e.g., antibody or antigen binding fragment thereof), polypeptide, polynucleotide, vector, or host cell provided herein.

In one aspect, a pharmaceutical composition provided herein comprises (1) an antigen binding polypeptide, antigen binding polypeptide complex (e.g., antibody or antigen binding fragment thereof), polypeptide, polynucleotide, vector, or host cell provided herein, and (2) a pharmaceutically acceptable carrier. The term “pharmaceutically acceptable carrier” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is known in the art. Except insofar as any conventional media or agent is incompatible with the active ingredient, its use in the therapeutic formulations is contemplated. Supplementary active ingredients can also be incorporated into the pharmaceutical compositions provided herein. In addition, various excipients, such as are commonly used in the art, can be included. These and other such compounds are described in the literature, e.g., in the Merck Index. Merck & Company. Rahway, NJ. Considerations for the inclusion of various components in pharmaceutical compositions are described, e.g., in Gilman et al. (Eds.) (2010); Goodman and Gilman's: The Pharmacological Basis of Therapeutics. 12th Ed., The McGraw-Hill Companies. In some aspects, the pharmaceutical composition is for parenteral, intravenous or subcutaneous administration.

In some aspects, a pharmaceutical composition provided herein comprises a polynucleotide or a vector disclosed herein (e.g., an mRNA disclosed herein) complexed, or packaged in a liposome, a nanoliposome, a lipid nanoparticle, a lipoplex, a micell, a nanomicell, a nanoemulsion, an oil-in-water emulsions, a PEG-conjugated lipid nanoparticle, a polymeric nanoparticle, a lipid-polymer hybrid nanoparticle, a polysaccharidic nanocarrier, an RNA-peptide nanoparticle, an RNA-peptide nanocomplex, a biomimetic nanovesicle, a lipidoid-RNA complex, a virus-like particle, dendrimer nanoparticle, a nanogel, a metallic nanoparticle, a gold nanoparticle (AuPNs), a magnetic nanoparticle, a theranostic nanoparticle, or any combination thereof.

In some aspects, a pharmaceutical composition provided herein comprises a lipid nanoparticle composition. As used herein, a “lipid nanoparticle composition.” “lipid nanoparticle formulation.” “LNP composition.” or “LNP formulation” is a composition comprising one or more lipids. The lipids in a lipid nanoparticle composition are typically sized on the order of micrometers or smaller. In some aspects, the lipids in a lipid nanoparticle composition have an average size of less than 1 micrometer.

In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid.

As used herein, a “cationic and/or ionizable lipid” is a lipid that has a positive or partial positive charge at physiological pH. Examples of a cationic and/or ionizable lipid include, but are not limited to, a lipid including a cyclic amine group, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2S)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), and mixtures thereof.

As used herein, a “structural lipid” is a fat synthesized from mixtures of long-chain and medium-chain fatty acids. A structural lipid is typically a triacylglycerol restructured or modified to change the fatty acid composition and/or positional distribution. Examples of a structural lipid include, but are not limited to, cholesterol, fecosterol, sitosterol, ergosterol, campesterol, stigmasterol, brassicasterol, tomatidine, tomatine, ursolic acid, alpha-tocopherol, and mixtures thereof.

As used herein, a “PEGylated lipid” is a lipid that has been modified to include polyethylene glycol or “PEG.” Examples of a PEGylated lipid include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, PEG-c-DOMG, PEG-DMG, PEG-DLPE, PEG-DMPE, PEG-DPPC, a PEG-DSPE lipid, and mixtures thereof.

As used herein, a “phospholipid” is a class of lipids whose molecule has a hydrophilic “head” containing a phosphate group and two hydrophobic “tails” derived from fatty acids, joined by an alcohol residue (usually a glycerol molecule). Examples of a phospholipid include, but are not limited to, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholestery lhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, the phospholipid is DSPC, In some aspects, the phospholipid is DOPE, In some aspects, the phospholipid is DSPC and DOPE.

Exemplary lipid nanoparticle compositions and methods for making them are described, for example, herein and in U.S. Pat. Nos. 11,524,023; 11,485,972; 10,898,574; 10,703,789; 10,702,600; 10,577,403; 10,442,756; 10,266,485; 10,064,959; 9,868,692; Int'l Pub. No, WO 2019/046809; Int'l Pub. No, WO 2020/160397; U.S. Pub. No. 2020/0306191; Xu et al., Adv. Nanobio. Res. 2:2, 2022; Cullis et al., Mol. Ther. 25(7):1467-1475, 2017; Fan et al., J. Pharm. Biomed. Anal. 192:113642, 2020; Paunovska et al., Nature Rev., 23:265-280, 2022; Buck et al., ACS Nano. 13:3754-3782, 2019; Pardi et al., Nature Comm. 8:14630, 2017; and Pardi et al., J. Control Release, 217:345-351, 2015, which are incorporated by reference herein. Such methods include, but are not limited to, back translating DNA coding for an antigen binding polypeptide or antigen binding polypeptide complex provided herein (e.g., an antibody or antigen binding fragment thereof) into an in vitro transcription (IVT) plasmid according to such published methods.

Exemplary compositions and methods related to mRNA preparation and delivery are described, for example, herein and in Int'l Pub. No, WO 2018/081638, Int'l Pub. No, WO 2017/182524, U.S. Pat. No. 9,012,219, Int'l Pub. No, WO 2016/176330, U.S. Pat. No. 9,371,511, U.S. Appl. Pub. No. 2018/0028645, U.S. Appl. Pub. No. 23018/0344838, U.S. Pub. No. 2018/0265848, U.S. Appl. Pub. No. 2017/0043037, U.S. Appl. Pub. No. 2017/0327842, U.S. Pat. No. 10,006,007, U.S. Appl. Pub. No. 2013/0261172, U.S. Appl. Pub. No. 2013/0197068, U.S. Appl. Pub. No. 2015/0038558, U.S. Appl. Pub. No. 2016/0032316, U.S. Appl. Pub. No. 2013/0111615, U.S. Appl. Pub. No. 2009/0286852, Cullis et al., Mol. Ther. 25(7):1467-1475, 2017; Pardi et al., Nature Comm. 8:14630, 2017; Pardi et al., J. Control Release, 217:345-351, 2015; and Grier et al., Mol. Ther. Nucleic Acids, 5:e306 (2016), which are incorporated by reference herein. Such methods include, but are not limited to, in vitro transcription (e.g., using PCR products or a linearized plasmid); IVT vectors; and synthesizing mRNA using, e.g., HiScribe T7 High Yield RNA Synthesis Kit (New England Biolabs), incorporating, e.g., Pseudouridine-5′-Triphosphate (TriLink BioTechnologies). In some aspects. 5′ capped mRNA can also be generated during this process using, e.g., CleanCap Reagent AG (TriLink BioTechnologies) to form, e.g., 100 bp or 120 bp poly(A) tails.

In some aspects, the pharmaceutical composition comprises (1) one or more polynucleotides encoding an antigen binding polypeptide or antigen binding polypeptide complex, and (2) a carrier. In some aspects, the pharmaceutical composition comprises (1) one or more polynucleotides encoding an antigen binding polypeptide or antigen binding polypeptide complex (e.g., antibody or antigen binding fragment thereof) provided herein, and (2) a carrier selected from the group consisting of a lipid or a lipid nanoparticle. In some aspects, the one or more polynucleotides encoding an antigen binding polypeptide or antigen binding polypeptide complex is a stabilized polynucleotide resistant to degradation or decomposition in vivo. In some aspects, the stabilized polynucleotide is capped at one end (5′ cap) and has a long polyadenylated (poly A) tail at the other end to form a stabilized mRNA having 5′ and 3′ UTRs. In some aspects, the poly A tail is about 50 bp, about 100 bp, about 120 bp, or about 150 bp. In some aspects, modified nucleosides may be incorporated into the mRNA to increase translation and/or to lower potential immunogenicity.

Also provided herein is a pharmaceutical composition comprising an antigen binding polypeptide, antigen binding polypeptide complex (e.g., antibody or antigen binding fragment thereof), polypeptide, polynucleotide, vector, or host cell provided herein, and an additional pharmaceutical agent.

In some aspects, the additional pharmaceutical agent is 25-hydroxyvitamin D, an agent that potentiates vitamin D action, an anti-viral agent, an anti-malarial agent, an antibiotic, or a combination thereof.

In some aspects, the additional pharmaceutical agent is 25-hydroxyvitamin D.

In some aspects, the agent that potentiates vitamin D action is a CYP24 inhibitor, 1,25-dihydroxyvitamin D compound, or a combination thereof.

In some aspects, the anti-viral agent is an anti-retroviral agent, an antibody or antigen binding fragment thereof, an inhibitor of reverse transcriptase, or a combination thereof.

In some aspects, the anti-viral agent is maraviroc, enfuvirtide, amantadine, lamivudine, nevirapine, efavirenz, dolutegravir, elvitegravir, raltegravir, acyclovir and any nucleoside analog of aciclovir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, sofobuvir, bebtelovimab (LY1404, LY-CoV1404, LY3853133), or a combination thereof.

In some aspects, the anti-viral agent is bebtelovimab.

In some aspects, the anti-viral agent is Retrovir® (3′-azido-3′-deoxypyrimidine, zidovudine), 3′-azido-3′-deoxythymidine (AZT), HMDR) (2′,3′-dideoxycytidine, zalcitabine), VidexEC® (2′,3′dideoxyinosine, didanosine), Epivir® (lamivudine), Zerit® (stavudine), Viread® (tenofovir DF), Ziagen® (abacavir), Emtriva® (emtricitabine, FTC), Rescriptor® (delavirdine), Sustiva® (efavirenz), Viramune® (nevirapine, 11-cyclopropyl-4-methyl-5,11-dihydro-6H-dipyrido [3,2-b:2′,3′-e][1,4]diazepin-6-one), trisodium phosphonoformate, ammonium-21-tungstenato-9-antimonate, 1-β-D-ribofuranoxyl-1,2,4-triazole-3-carboxamide, Aganerase® (amprenavir), Reyataz® (atazanavir), Lexiva® (fosamprenavir), Crixivan® (indinavir), Viracept® (nelfinavir), Norvir® (ritonavir), Fortovase® or Invirase® (saquinavir), lasinavir (5(S)-(tert-butoxycarbonylamino)-4(S)-hydroxy-6-phenyl-2(R) (2,3,4-trimethoxyphenylmethyl)-hexanoyl-(L)-valyl-N-(2-methoxy-ethyl)-amide), adriamycin, KVX-478, VX-478, 141W94, AG-1343, KNI-272, U-96988, BILA-2011 BS (palinavir), polymannoacetate, Fuzcon® (enfuvirtide, T-20), Epzicom® (abacavir and lamivudine), Trizivir® (abacavir, lamivudine and zidovudine), Truvada® (emtricitabine and tenofir DF), Combivir® (lamivudine and zidovudine), Kaletra® (lopinavir and ritonavir), bebtelovimab, or a combination thereof.

In some aspects, provided herein is a kit comprising an antigen binding polypeptide, antigen binding polypeptide complex (e.g., antibody or antigen binding fragment thereof), polypeptide, polynucleotide, vector, host cell, or pharmaceutical composition provided herein, or a combination thereof. Once a pharmaceutical composition has been formulated, it can be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. In some aspects, provided herein is a kit for producing a single-dose administration unit. In one aspect, the kit contains a first container having a dried antigen binding polypeptide or polypeptide complex provided herein (e.g., antibody or antigen binding fragment thereof) and a second container having an aqueous formulation. In some aspects, provided herein is a kit containing an antigen binding polypeptide or polypeptide complex provided herein (e.g., antibody or antigen binding fragment thereof) in a single and/or multi-chambered pre-filled syringe (e.g., liquid syringe and/or lyosyringe). In some aspects, the kit further contains components for intravenous or subcutaneous administration.

In some aspects, a kit provided herein comprises one or more antigen binding polypeptides or antigen binding polypeptide complexes (e.g., antibodies or antigen binding fragments thereof) provided herein (e.g., 1, 2, 3, 4, 5 or more). In some aspects, a kit provided herein comprises an antigen binding polypeptide or antigen binding polypeptide complexes (e.g., antibodies or antigen binding fragments thereof) provided herein.

In some aspects, a kit provided herein comprises one or more pharmaceutical compositions comprising an antigen binding polypeptide complex (e.g., antibody or antigen binding fragment thereof) provided herein (e.g., 1, 2, 3, 4, 5 or more). In some aspects, a kit provided herein comprises a pharmaceutical compositions comprising an antigen binding polypeptide complexes (e.g., antibody or antigen binding fragment thereof) provided herein. In some aspects, the pharmaceutical composition further comprises a pharmaceutically acceptable carrier.

In some aspects, a kit provided herein comprises (i) one or more antigen binding polypeptide complexes (e.g., antibodies or antigen binding fragments thereof) provided herein (e.g., 1, 2, 3, 4, 5 or more), and (ii) one or more additional pharmaceutical agents (e.g., 1, 2, 3, 4, 5 or more). In some aspects, a kit provided herein comprises (i) an antigen binding polypeptide complex (e.g., antibody or antigen binding fragment thereof) provided herein, and (ii) an additional pharmaceutical agent.

In some aspects, a kit provided herein comprises (i) one or more pharmaceutical compositions comprising an antigen binding polypeptide complex (e.g., antibody or antigen binding fragment thereof) provided herein (e.g., 1, 2, 3, 4, 5 or more), and (ii) one or more additional pharmaceutical agents (e.g., 1, 2, 3, 4, 5 or more). In some aspects, a kit provided herein comprises (i) a pharmaceutical composition comprising an antigen binding polypeptide complex (e.g., antibody or antigen binding fragment thereof) provided herein, and (ii) an additional pharmaceutical agent. In some aspects, the pharmaceutical composition further comprises a pharmaceutically acceptable carrier.

In some aspects, the kit further comprises instructions for use, for example, for any of the methods of use described herein (e.g., treating or preventing (i) a cancer or a tumor, (ii) an infectious disease that is not a sarbecovirus infectious disease, or (iii) a pathology other than a cancer or a tumor or an infectious disease that is not a sarbecovirus infectious disease).

### Methods of Use

In some aspects, provided herein are certain methods of use of an antigen binding polypeptide, antigen binding polypeptide complex (e.g., an antibody or antigen binding fragment thereof), polypeptide, polynucleotide, vector, host cell, or pharmaceutical composition provided herein, or a combination thereof. In some aspects, provided herein is a method of preventing or treating (i) a cancer or a tumor, (ii) an infectious disease that is not a sarbecovirus infectious disease, or (iii) a pathology other than a cancer or a tumor or an infectious disease that is not a sarbecovirus infectious disease, comprising administering an antigen binding polypeptide, antigen binding polypeptide complex (e.g., an antibody or antigen binding fragment thereof), polypeptide, polynucleotide, vector, host cell or pharmaceutical composition provided herein, or a combination thereof.

In some aspects, provided herein is a method of preventing or treating (i) a cancer or a tumor, (ii) an infectious disease that is not a sarbecovirus infectious disease, or (iii) a pathology other than a cancer or a tumor or an infectious disease that is not a sarbecovirus infectious disease in a subject in need thereof, comprising administering to the subject a therapeutically effective amount of an antigen binding polypeptide, antigen binding polypeptide complex (e.g., an antibody or antigen binding fragment thereof), polypeptide, polynucleotide, vector, host cell or pharmaceutical composition provided herein, or a combination thereof.

In some aspects, provided herein is a method of preventing or treating a viral infection (i.e., an infection caused by a virus) in a subject comprising administering to the subject an antigen binding polypeptide, antigen binding polypeptide complex (e.g., an antibody or antigen binding fragment thereof), polypeptide, polynucleotide, vector, host cell or pharmaceutical composition provided herein, or a combination thereof, wherein the virus is selected from the group consisting of influenza virus, respiratory syncytial virus (RSV), chlamydia, adenovirdiae, mastadeno virus, aviadenovirus, herpesviridae, herpes simplex virus 1, herpes simplex virus 2, herpes simplex virus 5, herpes simplex virus 6, leviviridae, levivirus, enterobacteria phase MS2, allolevirus, poxviridae, chordopoxvirinae, parapoxvirus, avipoxvirus, capripoxvirus, leporiipoxvirus, suipoxvirus, molluscipoxvirus, entomopoxvirinae, papovaviridae, polyomavirus, papillomavirus, paramyxoviridae, paramyxovirus, parainfluenza virus 1, mobillivirus, measles virus, rubulavirus, mumps virus, pneumonovirinae, pneumovirus, me tapneumo virus, avian pneumovirus, human metapneumovirus, picornaviridae, enterovirus, rhinovirus, hepatovirus, human hepatitis A virus, cardiovirus, andaptho virus, reoviridae, orthoreovirus, orbivirus, rotavirus, cypovirus, fijivirus, phytoreovirus, oryzavirus, retroviridae, mammalian type B retroviruses, mammalian type C retroviruses, avian type C retroviruses, type D retrovirus group, BLV-HTLV retroviruses, lentivirus, human immunodeficiency virus 1, human immunodeficiency virus 2, HTLV-I and -II viruses, herpes simplex E virus. Epstein Barr virus, cytomegalovirus, hepatitis virus (HCV, HAV, HBV, HDV, HEV), Toxoplasma gondii virus, Treponema pallidium virus, human T-lymphotrophic virus, encephalitis virus. West Nile virus. Dengue virus. Varicella Zoster Virus, rubeola, mumps, rubella, spumavirus, flaviviridae, hepatitis C virus, hepadnaviridae, hepatitis B virus, togaviridae, alphavirus sindbis virus, rubivirus, rubella virus, rhabdoviridae, vesiculovirus, lyssavirus, ephemerovirus, cytorhabdo virus, necleorhabdo virus, arenaviridae, arenavirus, lymphocytic choriomeningitis virus. Ippy virus, lassa virus, coronaviridae, coronavirus, and torovirus.

In these aspects, the antigen binding polypeptide or antigen binding polypeptide complex (e.g., an antibody or antigen binding fragment thereof) is to be understood to specifically bind to at least one epitope on at least one antigen of the virus causing the viral infection. Non-limiting examples of viral antigens are influenza virus neuraminidase, influenza virus hemagglutinin, human respiratory syncytial virus (RSV)-viral proteins, RSV F glycoprotein, RSV G glycoprotein, herpes simplex virus (HSV) viral proteins, herpes simplex virus glycoproteins gB, gC, gD, and gE, chlamydia MOMP and PorB antigens, core protein, matrix protein or other protein of Dengue virus, measles virus hemagglutinin, herpes simplex virus type 2 glycoprotein gB, poliovirus 1 VP1, envelope glycoproteins of HIV 1, hepatitis B surface antigen, diptheria toxin, streptococcus 24M epitope, gonococcal pilin, pseudorabies virus g50) (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein. Serpulina hydodysenteriae protective antigen, bovine viral diarrhea glycoprotein 55. Newcastle disease virus hemagglutinin-neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus, infectious bovine rhinotracheitis virus glycoprotein E, glycoprotein G, infectious laryngotracheitis virus, infectious laryngotracheitis virus glycoprotein G or glycoprotein I, a glycoprotein of La Crosse virus, neonatal calf diarrhoea virus. Venezuelan equine encephalomyelitis virus, punta toro virus, murine leukemia virus, mouse mammary tumor virus, hepatitis B virus core protein and hepatitis B virus surface antigen or a fragment or derivative thereof, antigen of equine influenza virus or equine herpes virus, including equine influenza virus type A/Alaska 91 neuraminidase, equine influenza virus typeA/Miami 63 neuraminidase, equine influenza virus type A/Kentucky 81 neuraminidase equine herpes virus type 1 glycoprotein B, and equine herpes virus type 1 glycoprotein D, antigen of bovine respiratory syncytial virus or bovine parainfluenza virus, bovine respiratory syncytial virus attachment protein (BRSV G), bovine respiratory syncytial virus fusion protein (BRSV F), bovine respiratory syncytial virus nucleocapsid protein (BRSVN), bovine parainfluenza virus type 3 fusion protein, bovine parainfluenza virus type 3 hemagglutinin neuraminidase, bovine E viral diarrhoea virus glycoprotein 48 and glycoprotein 53, glycoprotein E of Dengue virus, and glycoprotein E1E2 of human hepatitis C virus.

In some aspects, provided herein is a method of preventing or treating a viral infection (i.e., an infection caused by a virus, a viral infectious disease) that is not a sarbecovirus infectious disease in a subject comprising administering to the subject an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, the antigen binding polypeptide or antigen binding polypeptide complex (e.g., an antibody or antigen binding fragment thereof) is to be understood to specifically bind to at least one epitope on at least one antigen of a virus causing a viral infection (i.e., an infection caused by a virus, a viral infectious disease). In some aspects, the viral infectious disease antigen that is not a sarbecovirus antigen is a viral antigen selected from the group consisting of an influenza virus (A, B, C) antigen (e.g., influenza virus envelope proteins, for example, influenza virus neuraminidase (NA, N1, N2, N3, N4, N5, N6, N7, N8, N9, N10, N11), influenza virus hemagglutinin (H1, H2, H3, H4, H5, H6, H7, H8, H9, H10, H11, H12, H13, H14, H15, H16, H17, H18) (e.g., H1N1, H3N2, H5N1, H7N9, H9N2), Yamagata virus antigen, Victoria virus antigen, influenza virus structural proteins (e.g., M1, M2, capsid protein), influenza virus functional proteins (e.g., ribonucleoprotein (NP), primary interferon antagonist (NS1), nuclear export protein (NEP)) influenza virus accessory proteins (e.g., PB2, PB1, or PA), for example, anti-HA, anti-NA, anti-H1, anti-H3, anti-H5, anti-H7, anti-H9, anti-N1, anti-N2, anti-N9, anti-H1N1, anti-H3N2, anti-H5N1, anti-H7N9, anti-H9N2, anti-A protein, anti-B protein, anti-stem, anti-M1, anti-M2, anti-capsid, anti-NP, anti-NS1, anti-NEP, anti-PB1, anti-PB2, and anti-PA); a swine influenza virus antigen (e.g., swine flu hemagglutinin, swine flu neuraminidase); a classical swine fever (CSF) (hog colera) virus antigen; an equine influenza virus antigen (e.g., equine influenza virus typeA/Miami 63 neuraminidase, equine influenza virus type A/Kentucky 81 neuraminidase, equine influenza virus type A/Alaska 91 neuraminidase); parainfluenza viruses (e.g., bovine parainfluenza virus, bovine parainfluenza virus type 3 fusion protein, bovine parainfluenza virus type 3 hemagglutinin neuraminidase); a (human) respiratory syncytial virus (RSV)-viral antigen (e.g., RSV F glycoprotein, RSV G glycoprotein. F protein of respiratory syncytial virus, RSV fusion glycoprotein); a herpes simplex virus (HSV) antigen (e.g., herpes simplex virus glycoproteins gB, gC, gD, and gE, herpes simplex virus type 2 glycoprotein gB); a Dengue virus antigen (e.g., core protein, matrix protein, glycoprotein E of Dengue virus, or other protein of Dengue virus); a measles virus antigen (e.g., hemagglutinin); a poliovirus 1 antigen (e.g., poliovirus 1 proteins (VP1)), a HIV-1 antigen and HIV-2 antigen (e.g., HIV envelope proteins (e.g., envelope glycoproteins of HIV 1, HIV envelope glycoprotein (Env), HIV envelope glycoprotein gp160, HIV envelope surface glycoprotein gp120, HIV transmembrane envelope protein gp41), HIV structural proteins (e.g., p17, p24, p7, p55), HIV functional proteins (e.g., p66, HIV-1 protease (PR), p31), HIV accessory proteins (e.g., Nef, Tat, Rev, Vif, Vpr, Vpu)); a hepatitis virus (HAV, HBV, HCV, HDV, HEV) antigen (e.g., hepatitis B virus antigen (e.g., surface antigen, core protein), hepatitis C virus antigen (e.g., glycoprotein E1E2)); a rabies virus (Rabies lyssavirus) antigen (e.g., rabies virus glycoprotein, e.g., G glycoprotein); a pseudorabies virus antigen (e.g., pseudorabies virus g50) (gpD), pseudorabies virus II (gpB), pseudorabies virus III (gpC), pseudorabies virus glycoprotein H, pseudorabies virus glycoprotein E); a papillomavirus (HPV) antigen; an Epstein-Barr virus (EBV) (Gamma herpes virus 4) antigen; a Herpes simplex virus (HSV, HSV-1, HSV-2) antigen (e.g., human herpes virus 8, equine herpes virus antigen (e.g., type 1 glycoprotein B, type 1 glycoprotein D)); a Herpes zoster antigen; a Cytomegalovirus antigen (e.g., cytomegalovirus glycoprotein B); a Zika virus antigen; a Transmissible gastroenteritis virus (TGEV) antigen (e.g., glycoprotein 195, matrix protein); a rotavirus antigen (e.g., swine rotavirus glycoprotein 38); a parvovirus antigen (e.g., swine parvovirus capsid protein); a filoviruses (e.g., Marburg and Ebola) antigen; a bovine viral diarrhea virus antigen (e.g., glycoprotein 55); a Newcastle disease virus antigen (hemagglutinin, neuraminidase); an orthopoxvirus antigen; a coxsackievirus antigen and aphthovirus (foot and mouth disease virus) antigen; a yellow fever virus antigen; a Chikunga virus antigen; a Nipah virus antigen; an African swine fever virus antigen; a rhinotracheitis virus antigen (e.g., infectious bovine rhinotracheitis virus glycoprotein E, glycoprotein G); a laryngotracheitis virus antigen (e.g., infectious laryngotracheitis virus glycoprotein G or glycoprotein I); a La Crosse virus antigen (e.g., La Crosse virus glycoprotein); a neonatal calf diarrhoea virus antigen; a Venezuelan equine encephalomyelitis virus antigen; a punta toro virus antigen; a murine leukemia virus antigen; a mouse mammary tumor virus antigen; a bovine respiratory syncytial virus antigen (e.g., bovine respiratory syncytial virus attachment protein (BRSV G), bovine respiratory syncytial virus fusion protein (BRSV F), bovine respiratory syncytial virus nucleocapsid protein (BRSVN)); a bovine E viral diarrhoea virus antigen (e.g., glycoprotein 48 and glycoprotein 53); a metapneumovirus (HMPV) antigen; and an Ebola virus antigen (e.g., ebolavirus glycoprotein, e.g., Zaire ebolavirus glycoprotein).

In some aspects, provided herein is a method of preventing or treating a bacterial or parasite infection (i.e., an infection caused by a bacterium or parasite, a bacterial or parasite infectious disease) in a subject comprising administering to the subject an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, the antigen binding polypeptide or antigen binding polypeptide complex (e.g., an antibody or antigen binding fragment thereof) is to be understood to specifically bind to at least one epitope on at least one antigen of a bacterium or parasite causing a bacterial or parasite infection (i.e., an infection caused by a bacterium or parasite, a bacterial or parasite infectious disease). In some aspects, the bacterial or parasite infectious disease antigen is a bacterial or parasite antigen selected from the group consisting of a Plasmodium (e.g., Plasmodium falciparum. Plasmodium vivax, Plasmodium malariae, Plasmodium ovale, Plasmodium knowlesi) antigen, a Streptococcus (e.g., Streptococcus pneumoniae, Streptococcus pyogenes, Streptococcus agalactiae, Streptococcus mutans, Streptococcus viridans, Streptococcus anginosus, Streptococcus intermedius, Streptococcus constellatus) antigen (e.g., 24M epitope), a Neisseria gonorrhoeae antigen (e.g., gonococcal pilin), a Corynebacterium antigen (e.g., Corynebacterium diphtheria (diptheria toxin), Corynebacterium ulcerans, Corynebacterium pseudotuberculosis), Mycobacterium tuberculosis antigens. Brachyspira hyodysenteriae (Serpulina hydodysenteriae) antigen (e.g., Serpulina hydodysenteriae protective antigen), a Chlamydia antigen (e.g., Chlamydia trachomatis) (e.g., MOMP and PorB), a Mycoplasma sp. (e.g., Mycoplasma genitalium, Mycoplasma hyopneumoniae, Mycoplasma pneumonia) antigen, a Staphylococcus (e.g., Staphylococcus aureus, coagulase-negative staphylococci (CoNS), Staphylococcus aureus alpha toxin, Staphylococcus aureus bi-component leucocidin) antigen, a Klebsiella sp. antigen, an Escherichia coli antigen (e.g., E. coli shiga toxin type-2. E. coli shiga toxin type-1), a Bacillus sp. (e.g., Bacillus anthracis, e.g., Bacillus anthracis anthrax) antigen, a Pseudomonas aeruginosa antigen (e.g., Pseudomonas aeruginosa type III secretion system), an Enterococcus sp. antigen, an Enterobacter sp. antigen, a Proteus sp. antigen, an Actinobacter sp. antigen, a Mycobacterium avium (Mycobacterium avium complex (MAC)) antigen, a Salmonella bacteria antigen, a Clostridium difficile antigen, a Toxoplasma (e.g., Toxoplasma gondii) antigen, a Histoplasma antigen, a Candida antigen, a Coccidioides antigen, a Cryptococcus antigen, a Cryptosporidium antigen, and a Cystoisospora (e.g., Cystoisospora belli) antigen, and another antigen (e.g., endotoxins, lymphotoxins (e.g., lymphotoxin alpha LTA), phosphatidylserine. Hsp90, CCR5, lipoteichoic acid, clumping factor A).

For example, in some aspects any one or more of the VL and/or VH comprised in antigen binding polypeptides or antigen binding polypeptide complexes (e.g., an antibodies or antigen binding fragments thereof) disclosed herein comprise a light chain complementarity determining region 1 (LCDR1), LCDR2, and/or LCDR3, and a heavy chain complementarity determining region 1 (HCDR1), HCDR2, and/or HCDR3 of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

For example, in some aspects any one or more of the VL and/or VH comprised in antigen binding polypeptides or antigen binding polypeptide complexes (e.g., an antibodies or antigen binding fragments thereof) disclosed herein comprise a VL and/or a VH of an antibody selected from the group consisting of libivirumab, rmab, rivabazumab pegol, setoxaximab, suvizumab, suvratoxumab, sevirumab, pritoxaximab, porgaviximab, REGN-EB3, obiltoxaximab, gedivumab, foravirumab, larcaviximab, motavizumab, palivizumab, rafivirumab, regavirumab, tuvirumab, cosfroviximab, felvizumab, atidortoxumab, edobacomab, nebacumab, pateclizumab, raxibacumab, urtoxazumab, tosatoxumab, berlimatoxumab, CR6261, diridavumab, firivumab, lesofavumab, navivumab, exbivirumab, lenvervimab, bavituximab, bezlotoxumab, efungumab, leronlimab, nirsevimab, pagibaximab, panobacumab, tefibazumab, ansuvimab, atoltivimab, maftivimab, and odesivimab.

In some aspects, provided herein is a method of preventing or treating a cancer (e.g., a solid tumor or a blood cancer) in a subject comprising administering to the subject an antigen binding polypeptide, antigen binding polypeptide complex (e.g., an antibody or antigen binding fragment thereof), polypeptide, polynucleotide, vector, host cell or pharmaceutical composition provided herein.

In these aspects, the antigen binding polypeptide or antigen binding polypeptide complex (e.g., an antibody or antigen binding fragment thereof) is to be understood to specifically bind to at least one epitope on at least one antigen associated with cancer (e.g., a tumor-associated antigens (TAAs), tumor-specific antigens (TSAs), or neoantigens), or to at least one epitope on at least one antigen selected from the group consisting of, e.g., A2AR, APRIL, ATPDase, BAFF, BAFFR, BCMA. BlyS, BTK, BTLA, B7DC, B7H1, B7H2, B7H3, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL25 CCR3, CCR4, CD3, CD16A, CD19, CD20, CD24, CD27, CD28, CD30, CD38, CD39, CD40, CD40L, CD47, CD52, CD70, CD80, CD86, CD123, CD133, CD137, CD137L, CD160, CD272, CEACAM5, CLEC9, CLEC91, CRTH2, CSF-1, CSF-2, CSF-3, CXCL1, CXCL2, CXCL4, CXCL12, CXCL13, CXCR3, cMet, CTLA4, DLL3, DLL4, DNGR-1, E-cadherin, EGFR, ENTPD1, EpCAM, FCER1, FCER1A, FCER2, FGFR, FLAP, FOLH1, Gi24, GITR, GITRL, GPR5, GP100, GPRC5D, HER2, HER3, ICOSL, ICOS, HHLA2, HMGB1, HVEM, IDO, IFNa, IgE, IGF1R, IL2Rbeta, IL1, IL1A, IL1B, IL1F10, IL2, IL4, IL4Ra, IL5, ILSR, IL6, IL7, IL7Ra, IL8, IL9, IL9R, IL10, rhIL1O, IL12, IL13, IL13Ra1, IL13Ra2, IL15, IL17, IL17Rb, IL18, IL22, IL23, IL25, IL7, IL33, IL35, ITGB4, ITK, KIR, LAG3, LAMP1, leptin, LPFS2, MHC class II, MUC-1, MUC-16, NCR3LG1, NKG2D, NKp46, NTPDase-1, OX40, OX40L, PD-1, PD-L1, PD-L2, PROM1, S152, SIRPalpha, SISP1, SLC, SPG64, ST2, STEAP1, STEAP2, Syk kinase, STEAP1, TROP2, TACI, TDO, TGFBETA, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, and WUCAM.

In some aspects, provided herein is a method of preventing or treating a cancer or a tumor in a subject comprising administering to the subject an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, the antigen binding polypeptide or antigen binding polypeptide complex (e.g., an antibody or antigen binding fragment thereof) is to be understood to specifically bind to at least one epitope on at least one tumor-associated antigen (TAA) associated with the cancer or tumor. In some aspects, the TAA is selected from the group consisting of 5′-nucleotidase, 5T4, A2AR, activin receptor-like kinase 1, adenocarcinoma antigen, ADRB3, AFP, AKAP-4, ALK, alpha-fetoprotein, androgenreceptor, angiopoietin 2, AOC3, APRIL, ATPDase, AXL, B7-4, B7DC, B7H1, B7H2, B7H3, B7H4, B7H5, B7H6, B7H7, B7RP1, BAFF, BAFFR, BCMA, bcr-abl. BlyS, BORIS, BST2, BTK, BTLA, C3, C5, C5a, C5a receptor, CA-125, CAIX, CanAg (a glycoform of MUC1), CCL11, CCL15, CCL17, CCL19, CCL2, CCL20, CCL21, CCL25, CCL3, CCL4, CCL5, CCL7, CCL8, CCR2, CCR3, CCR4, CCR5, CD11, CD11a, CD123, CD125, CD133, CD134, CD137, CD137L, CD147, CD15, CD154, CD160, CD16A, CD171, CD179a, CD18, CD184, CD19, CD2, CD20, CD200, CD22, CD221, CD23, CD24, CD242, CD25, CD27, CD272, CD276, CD278, CD279, CD28, CD3, CD30, CD300LF, CD319, CD33, CD37, CD38, CD39, CD38, CD4, CD40, CD40L, CD41, CD44v6, CD45, CD47, CD49B, CD5, CD51, CD52, CD54, CD56, CD6, CD62L, CD7, CD70, CD72, CD74, CD79a, CD79b, CD80, CD86, CD97, CEA, CEACAM5, claudin 18 isoform 2, CLDN6, CLEC12A, CLEC9), CLEC91, CLL-1, cMet, coagulation factor III, CRTH2, CS1, CSF-1, CSFIR, CSF-2, CSF-3, CTGF, CTLA4, CXCL1, CXCL12, CXCL13, CXCL2, CXCL4, CXCR3, CXORF61, CyclinB1, CYP1B1, dendritic cell-associated lectin 2, DLL3, DLL4, DNGR-1, DR5, E7, E-cadherin, EGFL7, EGFR, EGFRVIII, ELF2M, EMR2, endoglin, ENTPD1, EpCAM, EphA2, EPHA3, ephrin receptor A3, EphrinB2, episialin, ERBB2 (Her2/neu), ERBB3, ERG (TMPRSS2-ETS fusion gene), ETV6-AML, FAP, FCAR, FCER1, FCER1A, FCER2, FCRL5, FGF 23, FGFR, FGFR2, fibronectin extra domain-B, FLAP, FLT3, folate hydrolase, folate receptor 1, folate receptor alpha, folate receptor beta, FOLH1, Fos-relatedantigen1, Frizzled receptor. Fucosyl GM1, GD2, GD3, gelatinase B. Gi24, GITR, GITRL, GloboH, Glutamate carboxypeptidase II, glypican 3, GM3, GP100, GPC1, GPC2, GPC3, gplOO, GPNMB, GPR20, GPR5, GPRC5D, growth differentiation factor 8, GUCY2C, HAVCR1, HER1, HER2, HER3, HGF, HGFR, HHLA2, histone complex, HLA-DR, HMGB1, HMWMAA, HPVE6, hTERT, human telomerase reverse transcriptase, HVEM, ICAM-1, ICOS, ICOSL, IDO, IFNa, IgE, IGF-1, IGF1R, IGF-2, IGF-I receptor, IGLL1, IL1, IL10, IL12, IL13, IL13Ra2, IL-13Ra2, IL13Ra1, IL15, IL17, IL-17A, IL-17AF, IL-17F, IL17Rb, IL18, IL1B, IL1F10, IL-1a, IL-1B, IL2, IL-20, IL22, IL23, IL-23A, IL25, IL2Rbeta, IL-3 receptor, IL-31 receptor A, IL33, IL35, IL4, IL4Ra, IL5, ILSR, IL6, IL7, IL7Ra, IL8, IL9, IL9R, IL1A, IL-llRa, integrin α4, integrin α4 β7, integrin α5β1, integrin αIIbβ3, integrin αvβ3, integrin β7, interleukin 36 receptor IL1RAP, interleukin 36 receptor IL1RL2, intestinal carboxylesterase, ITGB4, ITK, KIR, KIR2D, KIT, LAG3, LAGE-1a, LAIR1, LAMP1, LCK, legumain, leptin, LewisY, LILRA2, LIV-1, LMP2, LOXL2, LPFS2, LRRC15, LY6K, LY75, LYPD3, MAD-CT-1, MAD-CT-2, MAGEA1, MAGE-A1, MCAM, MCP-1, MelanA/MART1, mesothelin, MHC classII, MIF, ML-IAP, MS4A1, MST1R (RON), MUC1, MUC-1, MUC16, MUC-16, MUCSAC, muthsp70-2, MYCN, NA17, NCA-90) granulocyte antigen, NCAM, NCR3LG1, nectin-4, NGNA ganglioside, NKG2A, NKG2D, NKp46, Notch 1, Notch receptor, NRP1, NTPDase-1, NY-BR-1, NY-ESO-1, o-acetyl-GD2, OR51E2, OX40, OX40L, OY-TES1, p53, p53mutant, PANX3, PAP, PAX3, PAX5, PCDC1, PCDP1, PCTA-1/Galectin8, PD-1, PDGFRA, PDGFR-beta, PD-L1, PD-L2, phosphate-sodium co-transporter, phosphatidylserine, PLAC1, polysialicacid, PROM1, prostase, prostein, PRSS21, PSCA, PSMA, PTK7, RAGE-1, RANKL, Ras mutant, rhIL1O, RhoC, root plate-specific spondin 3, ROR1, ROR2, RTN4, RU1, RU2, S152, sarcoma translocation breakpoints, SART3, scatter factor receptor kinase, SDC1, SIRPalpha, SISP1, SLAMF7, SLC, sLe, SLITRK6, spermprotein17, SPG64, sphingosine-1-phosphate, SSEA-4, SSX2, ST2, STEAP1, STEAP2, surviving and telomerase, Syk kinase, T14, TACI, TAG72, TARP, T-cell receptor, TCRβ, TDO, TEM1/CD248, TEM7R, tenascin C, TGFBETA, TGF-β1, TGF-β2, TGS5, the extracellular portion of the APRIL protein. Tie2, TIGIT, TIM3, TLR, TLR2, TLR4, TLR5, TLR9, TMEF1, TnAg, TNF, TNFa, TNFR superfamily member 4, TNFRSF7, Tp55, TRAC, TRAIL-R1, TRAIL-R2, TRAP, TREM1, TROP2, TRP-2, TSHR, TSLP, TSLPR, tumor antigen CTAA16.88, TWEAK, tyrosinase, TYRP1 glycoprotein 75, UPK2, VAP-1, VEGF, VEGFA, VEGFR-1, VEGFR2, vimentin, VISTA, Vstm3, WT1, WUCAM, and XAGE1.

For example, in some aspects any one or more of the VL and/or VH comprised in antigen binding polypeptides or antigen binding polypeptide complexes (e.g., an antibodies or antigen binding fragments thereof) disclosed herein comprise a light chain complementarity determining region 1 (LCDR1), LCDR2, and/or LCDR3, and a heavy chain complementarity determining region 1 (HCDR1), HCDR2, and/or HCDR3 of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afascvikumab, afascvikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

For example, in some aspects any one or more of the VL and/or VH comprised in antigen binding polypeptides or antigen binding polypeptide complexes (e.g., an antibodies or antigen binding fragments thereof) disclosed herein comprise a VL and/or a VH of an antibody selected from the group consisting of losatuxizumab vedotin, utomilumab, urelumab, oleclumab, naptumomab estafenatox, ascrinvacumab, pintumomab, tacatuzumab tetraxetan, nesvacumab, vanucizumab, timolumab, vapaliximab, vepalimomab, enapotamab vedotin, ianalumab, belimumab, tabalumab, tibulizumab, belantamab mafodotin, teclistamab, teclistamab, detumomab, nacolomab tafenatox, eculizumab, lendalizumab, pexelizumab, pozelimab, ravulizumab, tesidolumab, vilobelimab, abagovomab, igovomab, oregovomab, sofituzumab vedotin, cantuzumab mertansine, cantuzuma bravtansine, girentuximab, altumomab pentetate, arcitumomab, besilesomab, bertilimumab, plozalizumab, mogamulizumab, leronlimab, PRO140, rovelizumab, rovelizumab, talacotuzumab, benralizumab, tavolimab, vonlerolizumab, gavilimomab, ziralimumab, fanolesomab, ipilimumab, ruplizumab, toralizumab, dapirolizumab pegol, toralizumab, dapirolizumab pegol, ruplizumab, blinatumomab, inebilizumab, loncastuximab tesirine, SGN-CD19A, tafasitamab, taplitumomab paptox, XMAB-5574, Budoprutug, Obexelimab, duvortuxizumab, englumafusp alfa, Emfizatamab, zeripatamig, siplizumab, blontuvetmab, FBTA05, ibritumomab tiuxetan, obinutuzumab, ocaratuzumab, ocrelizumab, ofatumumab, rituximab, tositumomab, ublituximab, veltuzumab, divozilimab, eramkafusp alfa, ripertamab, zuberitamab, glofitamab, nofetumomab merpentan, samalizumab, imvotamab, odronextamab, plamotamab, bectumomab, inotuzumab ozogamicin, moxetumomab pasudotox, pinatuzumab vedotin, gomiliximab, lumiliximab, inolimomab, inolimomab, daclizumab, camidanluma btesirine, basiliximab, varlilumab, omburtamab, enoblituzumab, vopratelimab, lulizumabpegol, TGN1412, muromonab-CD3, otelixizumab, teplizumab, visilizumab, epcoritamab, epcoritamab, brentuximab vedotin, iratumumab, azintuxizumab vedotin, gemtuzumab ozogamicin, lintuzumab, vadastuximab talirine, naratuximab emtansine, otlertuzumab, lilotomab satetraxetan, tetulomab, daratumumab, isatuximab, foralumab, mosunetuzumab, mosunetuzumab, cedelizumab, ibalizumab, keliximab, priliximab, tregalizumab, zanolimumab, bleselumab, iscalimab, lucatumumab, ravagalimab, selicrelumab, teneliximab, vanalimab, abciximab, bivatuzumab, iomab-B, telimomab aritox, zolimomab aritox, abituzumab, intetumumab, alemtuzumab, tamtuvetmab, lorvotuzumab mertansine, itolizumab, cusatuzumab, vorsetuzumab mafodotin, milatuzumab, iladatuzumab vedotin, polatuzumab vedotin, galiximab, cibisatamab, zolbetuximab, naxitamab, tisotumab vedotin, olendalizumab, pamrevlumab, lacnotuzumab, cabiralizumab, emactuzumab, axatilimab, gimsilumab, lenzilumab, namilumab, tremelimumab, ulocuplumab, tepoditamab, rovalpituzumab tesirine, tarlatamab, demcizumab, navicixizumab, drozitumab, parsatuzumab, depatuxizumab mafodotin, modotuximab, carotuximab, adecatumumab, adecatumumab, oportuzumab monatox, solitomab, tucotuzumab celmoleukin, edrecolomab, catumaxomab, ifabotuzumab, fibatuzumab, zalutumumab, panitumumab, cetuximab, futuximab, imgatuzumab, laprituximab emtansine, matuzumab, necitumumab, nimotuzumab, tomuzotuximab, amivantamab, amivantamab, epitumomab cituxetan, sontuzumab, duligotuzumab, elgemtumab, lumretuzumab, patritumab, seribantumab, zenocutuzumab, sibrotuzumab, burosumab, aprutumab ixadotin, radretumab, pasotuxizumab, farletuzumab, mirvetuximab soravtansine, vantictumab, TRBS07, 3F8, dinutuximab, dinutuximab beta, ecromeximab, mitumomab, andecaliximab, capromab, codrituzumab, glembatumumab vedotin, talquetamab, talquetamab, trevogrumab, indusatumab vedotin, ficlatuzumab, rilotumumab, zatuximab, pentuzimab, pentuzimab, margetuximab, timigutuzumab, trastuzumab, trastuzumab duocarmazine, trastuzumab emtansine, gancotamab, pertuzumab, ertumaxomab, emibetuzumab, telisotuzumab, telisotuzumab vedotin, derlotuximab biotin, apolizumab, onartuzumab, bersanlimab, enlimomabpegol, rontalizumab, sifalimumab, talizumab, ganitumab, istiratumab, robatumumab, teprotumumab, figitumumab, ganitumab, istiratumab, robatumumab, teprotumumab, cixutumumab, dalotuzumab, xentuzumab, dusigitumab, canakinumab, ustekinumab, ustekinumab, ustekinumab, briakinumab, briakinumab, abrezekimab, lebrikizumab, romilkimab, TNX-650, tralokinumab, anrukinzumab, brodalumab, ixekizumab, netakimab, perakizumab, secukinumab, vunakizumab, afasevikumab, afasevikumab, bimekizumab, bimekizumab, remtolumab, bermekimab, lutikizumab, lutikizumab, gevokizumab, cergutuzumab amunaleukin, fletikumab, fezakinumab, brazikumab, guselkumab, mirikizumab, tildrakizumab, risankizumab, flotetuzumab, nemolizumab, pascolizumab, dupilumab, mepolizumab, reslizumab, clazakizumab, elsilimomab, olokizumab, sarilumab, siltuximab, sirukumab, vobarilizumab, tocilizumab, SA237, satralizumab, enokizumab, prezalizumab, natalizumab, abrilumab, abrilumab, vedolizumab, volociximab, tadocizumab, etaracizumab, etrolizumab, spesolimab, vatelizumab, erlizumab, lirilumab, relatlimab, cBR96-doxorubicinimmunoconjugate, odulimomab, efalizumab, ladiratuzumab vedotin, simtuzumab, samrotamab vedotin, aselizumab, lupartumab amadotin, imalumab, carlumab, imaprelimab, amatuximab, amatuximab, anetumab ravtansine, narnatumab, clivatuzumab tetraxetan, pemtumomab, gatipotuzumab, ensituximab, lemalesomab, sulesomab, enfortumab vedotin, racotumomab, monalizumab, brontictuzumab, tarextumab, vesencumab, oxelumab, gilvetmab, tislelizumab, dostarlimab, BCD-100, nivolumab, PDR001, pembrolizumab, pidilizumab, retifanlimab, spartalizumab, sintilimab, cemiplimab, olaratumab, tovetumab, rinucumab, atezolizumab, avelumab, durvalumab, lifastuzumab vedotin, bavituximab, acapatamab, capromab, rosopatamab, nezastomig, pasotuxizumab, pelgifatamab, voxalatamab, cofetuzumab pelidotin, denosumab, rosmantuzumab, cirmtuzumab, atinumab, indatuximab ravtansine, elotuzumab, sirtratumab vedotin, sonepcizumab, vandortuzumab vedotin, minretumomab, satumomab pendetide, maslimomab, ontuxizumab, tenatumomab, fresolimumab, metelimumab, lerdelimumab, tezepelumab, etigilimab, tiragotumab, placulumab, prezalumab, infliximab, nerelimomab, ozoralizumab, adalimumab, afelimomab, certolizumab pegol, golimumab, mapatumumab, conatumumab, lexatumumab, tigatuzumab, sacituzumab govitecan, votumumab, pogalizumab, letolizumab, pankomab, anatumomab mafenatox, enavatuzumab, flanvotumab, brolucizumab, ranibizumab, varisacumab, bevacizumab, faricimab, icrucumab, alacizumab pegol, ramucirumab, pritumumab, and onvatilimab.

In some aspects, provided herein is a method of preventing or treating a pathology other than a cancer or a tumor or an infectious disease that is not a sarbecovirus infectious disease in a subject comprising administering to the subject an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, the antigen binding polypeptide or antigen binding polypeptide complex (e.g., an antibody or antigen binding fragment thereof) is to be understood to specifically bind to at least one epitope on at least one other-pathology antigen associated with the pathology other than a cancer or a tumor or an infectious disease that is not a sarbecovirus infectious disease. In some aspects, the other-pathology antigenes selected from the group consisting of Amyloid beta, β-amyloid, PCSK9, IFN-α/β receptor, Rhesus factor, nerve growth factor (NGF), DPP4, fibrin II beta chain, ACVR2B, serum amyloid A protein SOST, FGF 23, VWF, tissue factor pathway inhibitor (TFPI), 1-40 and 1-42, selectin P, glucagon receptor (GCGR), amyloid P component, GDF-8, CXCL10) IP-10, repulsive guidance molecule A RGMA, IFN-γ, activated F9/F10, calcitonin gene-related peptide (CGRP), angiopoietin 3, IFN receptor, IFN-γ, calcitonin, β-amyloid 1-40 and 1-42, tau protein, dabigatran, cardiac myosin, selectin P, Complement factor D (CFD), kallikrein, GDF-8, IGHE, tissue factor pathway inhibitor (TFPI), GMCSF receptor α-chain, amyloid, IgE Fc region, MASP-2, oxLDL, GMCSF, NOGO-A. Alpha-synuclein, NGF, myelin-associated glycoprotein, RHD (gene) (RHD), sclerostin, FCGRT, sclerostin SOST, mucosal addressin cell adhesion molecule, myostatin, RSVFR, complement component 1s C1s, C5, and IL31.

For example, in some aspects any one or more of the VL and/or VH comprised in antigen binding polypeptides or antigen binding polypeptide complexes (e.g., an antibodies or antigen binding fragments thereof) disclosed herein comprise a light chain complementarity determining region 1 (LCDR1), LCDR2, and/or LCDR3, and a heavy chain complementarity determining region 1 (HCDR1), HCDR2, and/or HCDR3 of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

For example, in some aspects any one or more of the VL and/or VH comprised in antigen binding polypeptides or antigen binding polypeptide complexes (e.g., an antibodies or antigen binding fragments thereof) disclosed herein comprise a VL and/or a VH of an antibody selected from the group consisting of aducanumab, alirocumab, frovocimab, lodelcizumab, bococizumab, evolocumab, ralpancizumab, anifrolumab, atorolimumab, bapineuzumab, bedinvetmab, begelomab, biciromab, bimagrumab, birtamimab, blosozumab, burosumab, caplacizumab, concizumab, crenezumab, crizanlizumab, crotedumab, dezamizumab, domagrozumab, donanemab, eldelumab, elezanumab, emapalumab, emicizumab, eptinezumab, erenumab, evinacumab, faralimomab, fasinumab, fontolizumab, fremanezumab, frunevetmab, fulranumab, galcanezumab, gantenerumab, gosuranemab, idarucizumab, imciromab, inclacumab, lampalizumab, lanadelumab, landogrozumab, lecanemab, ligelizumab, marstacimab, mavrilimumab, morolimumab, NEOD001, omalizumab, oMS721, orticumab, otilimab, ozanezumab, ponezumab, prasinezumab, quilizumab, ranevetmab, refanezumab, roledumab, romosozumab, rozanolixizumab, setrusumab, SHP647, solanezumab, stamulumab, suptavumab, sutimlimab, tanezumab, crovalimab, and lokivetmab.

Non-limiting examples of pathologies other than a cancer or a tumor or an infectious disease that is not a sarbecovirus infectious disease are Alzheimer's disease, cardiovascular disease, hypercholesterolemia, dyslipidemia, lupus erythematosus, hemolytic disease of the newborn, pain associated with osteoarthritis (e.g., in dogs), severe refractory idiopathic inflammatory myopathy, thromboembolism, pathological muscle loss and weakness, amyloidosis, osteoporosis, hypophosphatemia, thrombotic thrombocytopenia purpura, thrombosis, bleeding with hemophilia, sickle-cell disease, diabetes. Duchenne muscular dystrophy. Crohn's disease, ulcerative colitis, spinal cord injury, multiple sclerosis, hemophagocytic lymphohistiocytosis, haemophilia A, migraine, acute sciatic pain, pain associated with osteoarthritis (e.g., in cats), pain, progressive supranuclear palsy, reversal of anticoagulant effects of dabigatran, geographic atrophy secondary to age-related macular degeneration, angioedema), muscle wasting disorders, severe asthma, chronic spontaneous urticaria, rheumatoid arthritis, immune disease, primary systemic amyloidosis, allergic asthma, chronic spontaneous urticaria, atypical hemolytic uremic syndrome, coronary inflammation, osteoarthritis. Parkinson's disease, asthma, recovery of motor function after stroke. Rh disease, myasthenia gravis. Osteogenesis Imperfecta, muscular dystrophy, medically attended lower respiratory disease, cold agglutinin disease, paroxysmal nocturnal hemoglobinuria, and atopic dermatitis (e.g. in dogs).

Delivery of the antigen binding polypeptide, antigen binding polypeptide complex (e.g., antibody or antigen binding fragment thereof), polypeptide, or antibody or antigen binding fragment thereof provided herein can be by direct administration of the antigen binding polypeptide, antigen binding polypeptide complex (e.g., antibody or antigen binding fragment thereof), polypeptide, or antibody or antigen binding fragment thereof provided herein or by an indirect method that is inclusive of delivery of a modified mRNA composition that is administered to the patient in need of treatment thereof by an antigen binding polypeptide, an antigen binding polypeptide complex (e.g., antibody or antigen binding fragment thereof), a polypeptide, or an antibody or antigen binding fragment thereof provided herein translated from the modified mRNA that codes for the antigen binding polypeptide, antigen binding polypeptide complex (e.g., antibody or antigen binding fragment thereof), polypeptide, or antibody or antigen binding fragment thereof provided herein.

As used herein, the terms “prevent” or “preventing” refer to the prevention of the onset, recurrence or spread, in whole or in part, of a disease or condition provided herein, or a symptom thereof. As used herein, the terms “treat” or “treating” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treat” can also mean prolonging survival as compared to expected survival if not receiving treatment.

As used herein. “administering” is meant a method of giving a dosage of an antigen binding polypeptide or polypeptide complex (e.g., antibody or antigen binding fragment thereof) to a subject in need thereof (e.g., a patient). Administering can be by any suitable means, including parenteral, intrapulmonary or intranasal. Parenteral infusions include, for example, intramuscular, intravenous, intraarterial, intraperitoneal or subcutaneous administration. Dosing can be by any suitable route, e.g., by injection, such as intravenous or subcutaneous injection. Various dosing schedules including, but not limited to, single or multiple administrations over various time-points, bolus administration, and pulse infusion are contemplated herein.

As used herein, a “therapeutically effective amount” is an amount of an antigen binding polypeptide or antigen binding polypeptide complex (e.g., an antibody or antigen binding fragment thereof) that is sufficient to achieve the desired effect and can vary according to the nature and severity of the condition, and the potency of the polypeptide or polypeptide complex. In one aspect, an antigen binding polypeptide or polypeptide complex can be delivered by administering a polynucleotide, vector, or host cell that encodes the antigen binding polypeptide or polypeptide complex. In another aspect, an antigen binding polypeptide or polypeptide complex thereof can be delivered by administering a pharmaceutical composition containing the polypeptide or polypeptide complex. A therapeutic effect is the relief, to at least some extent, of one or more symptoms of the disease or disorder, and can include curing a disease or disorder. “Curing” means that the symptoms of active disease are eliminated. However, certain long-term or permanent effects of a disease or disorder can exist even after a cure is obtained.

As used herein, the term “subject” means a human or a non-human mammal, e.g., a dog, cat, mouse, rat, cow, sheep, pig, goat, non-human primate or bird, e.g., chicken, as well as any other vertebrate or invertebrate. In one aspect, the subject is a human. In another aspect, the subject is a veterinary animal. In one aspect, the subject is a mammal.

In some aspects, provided herein are methods of preventing or treating (i) a cancer or a tumor, (ii) an infectious disease that is not a sarbecovirus infectious disease, or (iii) a pathology other than a cancer or a tumor or an infectious disease that is not a sarbecovirus infectious disease, comprising administering to the subject a therapeutically effective amount of the antigen binding polypeptides or antigen binding polypeptide complexes, the antibodies or antigen binding fragments thereof, the polynucleotides, the vectors, the host cells, the mRNAs, the LNPs, the CARs, the immune cells, or the pharmaceutical compositions disclosed herein, or any combination thereof.

In some aspects, provided herein are methods of diagnosing a subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptides or antigen binding polypeptide complexes or the antibodies or antigen binding fragments thereof disclosed herein; (b) detecting the presence or absence of a complex which contains the polypeptide or a fragment thereof, polypeptide complex or a fragment thereof, or antibody or antigen binding fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is detected.

In some aspects, provided herein are methods of diagnosing a subject as not having or as not being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptides or antigen binding polypeptide complexes or the antibodies or antigen binding fragments thereof disclosed herein; (b) detecting the presence or absence of a complex which contains the polypeptide or a fragment thereof, polypeptide complex or a fragment thereof, or antibody or antigen binding a fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as not having or as not being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is not detected.

The term “sample.” as used herein, refers to a composition that is obtained or derived from a subject that contains a cellular and/or other molecular entity that is to be characterized and/or identified, for example, based on physical, biochemical, chemical, and/or physiological characteristics. A sample includes, but is not limited to, nasal fluid or discharge (e.g., from a nasal swab), tissue, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, tissue culture medium, tissue extracts such as homogenized tissue, cellular extracts, and combinations thereof.

Methods for collecting, processing and storing a sample are known, and described, for example, in Vaught et al., IARC Sci. Pub., Unit 2. Chapter 3, pp. 23-42, 2011.

Methods for detecting the binding of an antigen binding polypeptide or polypeptide complex (e.g., antibody or antigen binding fragment thereof), or fragments thereof, provided herein and an antigen or fragment thereof are also known. Such methods include, but are not limited to, immunosorbent assay (ELISA), sodium dodecyl sulfate-polyacrylamide gel electrophoresis (SDS-PAGE), immunospot assay, lateral flow assay, flow cytometry, immunohistochemistry or western blot. See, e.g., Kumar et al., VirusDisease, 31:97-105, 2020; Gong et al., Front. Mol. Biosci. Jul. 23, 2021; and Espejo et al., Am. J. Clin. Pathol., 154 (3) 293-304, 2020.

The following sequences are included as part of the present disclosure.

In any one of the aspects disclosed herein, when reference is made to SEQ ID NO: 795, it is to be understood that “X” in SEQ ID NO: 795 is a “T” when referring to the LCDR3 of antibody E5, and “X” in SEQ ID NO: 795 is a “P” when referring to the LCDR3 of antibody H9.

Lengthy table referenced here
US20250326823A1-20251023-T00001
Please refer to the end of the specification for access instructions.

Lengthy table referenced here
US20250326823A1-20251023-T00002
Please refer to the end of the specification for access instructions.

### EXAMPLES

The following examples are provided to further illustrate aspects of the disclosure, and are not meant to constrain the disclosure to any particular application or theory of operation.

### Example 1

### Pseudovirus Neutralization Assay of Anti-SARS-CoV-2 Monoclonal Antibodies

For selecting the candidates to be used for the design and production of the antigen binding polypeptides and antigen binding polypeptide complexes described herein, the pseudotyped neutralization potency (IC50 and IC80) of several anti-SARS-CoV-2 monoclonal antibodies was determined. Specifically. S-containing lentiviral pseudovirions were produced by co-transfection of packaging plasmid pCMVdR8.2, transducing plasmid pHR′ CMV-Luc, a TMPRSS2 plasmid and S plasmids from SARS-CoV-2 variants into 293T cells using Lipofectamine 3000 transfection reagent (L3000-001. ThermoFisher Scientific, Asheville, NC), 293T-ACE2 cells (provided by Dr. Michael Farzan) were plated into 96-well white/black Isoplates (PerkinElmer, Waltham, MA) at 7.500 cells per well the day before infection with SARS-CoV-2 pseudoviruses. Serial dilutions of the anti-SARS-CoV-2 monoclonal antibodies were mixed with titrated pseudoviruses, incubated for 45 minutes at 37° C., and added to cells in triplicates. Following 2 h of incubation, wells were replenished with 150 μl of fresh media. Cells were lysed 72 h later, and luciferase activity was measured with Microbeta (Perking Elmer). Percent neutralization and neutralization IC50 and IC80 values are shown in Tables E1-E3A-3B. Eight well performing anti-SARS-CoV-2 monoclonal antibodies (B8, E12, E8, A23-58, B1, G11, B2 and A18-448) were selected to be used in the design and production of the antigen binding polypeptides and antigen binding polypeptide complexes described herein. In addition, a ninth antibody previously shown to possess neutralization activity (A19-46) was also used in the design and production of the antigen binding polypeptides and antigen binding polypeptide complexes described herein.

TABLE E1
pseudotyped neutralization potency (IC50 and IC80) for the tested anti-SARS-CoV-2
monoclonal antibodies (IC50 and IC80 are expressed in μg/ml).

| | | BA.1 | | BA.4/5 | | BA.2.12.1 | |
|---|---|---|---|---|---|---|---|
| | mAbs | IC50 | IC80 | IC50 | IC80 | IC50 | IC80 |
| Class I | SARS2.E76_B8 | 0.009 | 0.037 | 0.003 | 0.010 | not tested | |
| (New) | SARS2.F768_pt2_G10 | 0.006 | 0.019 | 0.004 | 0.010 | 0.008 | 0.022 |
| | SARS2.F768_pt1_D11 | 0.035 | 0.151 | 0.002 | 0.008 | not tested | |
| | SARS2.F769_pt1_E12 | 0.004 | 0.011 | 0.002 | 0.011 | 0.002 | 0.008 |
| | SARS2.F768_pt1_A05 | 0.012 | 0.024 | 0.004 | 0.012 | 0.014 | 0.028 |
| | SARS2.F768_pt2_D12 | 0.002 | 0.009 | 0.006 | 0.021 | 0.020 | 0.053 |
| Class I | 58.1H/58.1LC_94GLTG | 0.004 | 0.012 | 0.005 | 0.030 | 0.002 | 0.009 |
| (Comparators) | 58.1H/58.1LC_93VGLTG | 0.003 | 0.011 | 0.003 | 0.014 | 0.003 | 0.011 |
| Class III | SARS2.F770_pt1_E8 | 0.003 | 0.022 | 0.0007 | 0.003 | 0.001 | 0.0014 |
| (New) | SARS2.F770_pt1_G11 | 0.030 | 0.114 | 0.014 | 0.069 | 0.046 | 0.138 |
| | SARS2.F769_pt1_B1 | 0.0013 | 0.003 | 0.0007 | 0.002 | 0.002 | 0.004 |
| | SARS2.F770_pt1_C1 | 0.008 | 0.036 | 0.0006 | 0.002 | 0.0007 | 0.002 |
| Class III | LY-CoV-1404 | 0.002 | 0.005 | 0.0014 | 0.003 | 0.0013 | 0.004 |
| (Comparators) | | | | | | | |
| | | BA.2.75 | | BA.4.6 | | BA.2.75.2 | |
| | mAbs | IC50 | IC80 | IC50 | IC80 | IC50 | IC80 |
| Class I | SARS2.E76_B8 | 0.006 | 0.031 | 0.0007 | 0.004 | 0.004 | 0.031 |
| (New) | SARS2.F768_pt2_G10 | 0.025 | 0.058 | 0.002 | 0.008 | 0.009 | 0.024 |
| | SARS2.F768_pt1_D11 | 0.012 | 0.030 | 0.003 | 0.009 | 0.009 | 0.028 |
| | SARS2.F769_pt1_E12 | 0.007 | 0.036 | 0.002 | 0.006 | 0.003 | 0.032 |
| | SARS2.F768_pt1_A05 | 0.018 | 0.032 | 0.002 | 0.008 | 0.010 | 0.032 |
| | SARS2.F768_pt2_D12 | 0.014 | 0.046 | 0.005 | 0.015 | 0.030 | 0.085 |
| Class I | 58.1H/58.1LC_94GLTG | 0.0010 | 0.003 | 0.003 | 0.011 | >10 | >10 |
| (Comparators) | 58.1H/58.1LC_93VGLTG | 0.0009 | 0.003 | 0.003 | 0.011 | >10 | >10 |
| Class III | SARS2.F770_pt1_E8 | 0.002 | 0.009 | 0.0007 | 0.0013 | 0.0012 | 0.011 |
| (New) | SARS2.F770_pt1_G11 | 0.044 | 0.094 | 0.017 | 0.072 | 0.072 | 0.224 |
| | SARS2.F769_pt1_B1 | 0.006 | 0.040 | 0.0007 | 0.002 | 0.007 | 0.028 |
| | SARS2.F770_pt1_C1 | 0.011 | 0.073 | 0.0006 | 0.002 | 0.002 | 0.008 |
| Class III | LY-CoV-1404 | 0.003 | 0.015 | 0.0004 | 0.0014 | 0.001 | 0.005 |
| (Comparators) | | | | | | | |
| | | BQ.1.1 | | BJ.1 | | XBB | |
| | mAbs | IC50 | IC80 | IC50 | IC80 | IC50 | IC80 |
| Class I | SARS2.E76_B8 | 0.021 | 0.059 | 0.009 | 0.034 | 0.008 | 0.045 |
| (New) | SARS2.F768_pt2_G10 | 0.014 | 0.036 | 0.006 | 0.029 | 0.007 | 0.025 |
| | SARS2.F768_pt1_D11 | 0.016 | 0.040 | 0.005 | 0.020 | 0.007 | 0.040 |
| | SARS2.F769_pt1_E12 | 0.011 | 0.038 | 0.002 | 0.023 | 0.013 | 0.042 |
| | SARS2.F768_pt1_A05 | 0.033 | 0.064 | 0.002 | 0.020 | 0.011 | 0.024 |
| | SARS2.F768_pt2_D12 | 0.029 | 0.080 | 0.002 | 0.010 | 0.048 | 0.175 |
| Class I | 58.1H/58.1LC_94GLTG | 0.015 | 0.046 | 0.003 | 0.009 | >10 | >10 |
| (Comparators) | 58.1H/58.1LC_93VGLTG | 0.013 | 0.032 | 0.002 | 0.007 | >10 | >10 |
| Class III | SARS2.F770_pt1_E8 | 0.072 | 0.448 | 0.033 | 0.264 | 0.044 | 0.094 |
| (New) | SARS2.F770_pt1_G11 | 0.874 | 2.5 | 0.032 | 0.165 | 0.058 | 0.171 |
| | SARS2.F769_pt1_B1 | 0.003 | 0.013 | 1.0 | 1.9 | 1.3 | 2.8 |
| | SARS2.F770_pt1_C1 | >10 | >10 | >10 | >10 | >10 | >10 |
| Class III | LY-CoV-1404 | >10 | >10 | >10 | >10 | >10 | >10 |
| (Comparators) | | | | | | | |

TABLE E2
pseudotyped neutralization potency (IC50 and IC80) for the tested anti-SARS-CoV-2
monoclonal antibodies (IC50 and IC80 are expressed in μg/ml; n.t.: not tested).

| | SARS-CoV-2 | | | | | | |
|---|---|---|---|---|---|---|---|
| | | D614G | | BA.4/5 | | BQ.1.1 | |
| mAbs | Class | IC50 | IC80 | IC50 | IC80 | IC50 | IC80 |
| F769-E12 | I | 0.012 | 0.028 | 0.0024 | 0.011 | 0.013 | 0.028 |
| F770-G11 | II | 0.014 | 0.028 | 0.014 | 0.069 | 0.874 | 2.5 |
| A80-102_G5 | I/IV | 0.169 | 0.395 | 4.8 | 9.6 | 3.1 | 9.1 |
| A18-448.1 | I/IV | 0.014 | 0.023 | 0.0038 | 0.0093 | 0.0095 | 0.023 |
| F768-104_B2 | III | 0.0054 | 0.015 | 0.0094 | 0.044 | 0.515 | 0.982 |
| F770-E8 | III | 0.0007 | 0.0028 | 0.0007 | 0.0026 | 0.139 | 0.563 |
| F768-D11 | I | 0.011 | 0.019 | 0.0018 | 0.0085 | 0.016 | 0.040 |
| E184-105_F3 | I | 0.0028 | 0.011 | 0.0025 | 0.0085 | 0.014 | 0.056 |
| A18-452.1 | IV | 0.697 | 1.97 | 1.81 | 2.09 | 1.81 | 5 |
| SARS2.A63-652-1.4 | I/IV | 0.011 | 0.019 | 0.0050 | 0.0090 | 0.015 | 0.028 |
| | SARS-CoV-2 | | | | | | |
| | | XBB.1.5 | | XBB.1.16 | | XBC.1 | |
| mAbs | Class | IC50 | IC80 | IC50 | IC80 | IC50 | IC80 |
| F769-E12 | I | 0.0068 | 0.032 | 0.0049 | 0.028 | 0.0022 | 0.0057 |
| F770-G11 | II | 0.086 | 0.315 | 0.103 | 0.308 | 0.017 | 0.047 |
| A80-102_G5 | I/IV | 3.5 | 8.6 | 2.6 | 5.1 | 2.2 | 5.1 |
| A18-448.1 | I/IV | 0.0039 | 0.011 | 0.0022 | 0.010 | 0.0021 | 0.0063 |
| F768-104_B2 | III | 0.244 | 0.510 | 0.175 | 0.410 | 0.0088 | 0.016 |
| F770-E8 | III | 0.035 | 0.171 | 0.094 | 0.348 | 0.0014 | 0.0066 |
| F768-D11 | I | 0.0086 | 0.021 | 0.0088 | 0.023 | 0.0026 | 0.0075 |
| E184-105_F3 | I | 0.047 | 0.076 | 0.017 | 0.064 | 0.0031 | 0.0037 |
| A18-452.1 | IV | 1.79 | 2.22 | n.t. | n.t. | n.t. | n.t. |
| SARS2.A63-652-1.4 | I/IV | 0.0080 | 0.015 | n.t. | n.t. | n.t. | n.t. |

| | | SARS-CoV-2 | | | |
|---|---|---|---|---|---|
| | | XBB.2.3.2 (SAVE) | | EG.5.1 | |
| mAbs | Class | IC50 | IC80 | IC50 | IC80 |
| F769-E12 | I | 0.063 | 0.110 | 0.138 | 0.562 |
| F770-G11 | II | 0.213 | 0.582 | 0.110 | 0.286 |
| A80-102_G5 | I/IV | 2.9 | 7.9 | 2.0 | 2.9 |
| A18-448.1 | I/IV | 0.0082 | 0.030 | 0.0026 | 0.0100 |
| F768-104_B2 | III | 0.225 | 0.723 | 0.066 | 0.165 |
| F770-E8 | III | 0.298 | 5.096 | 0.084 | 1.056 |
| F768-D11 | I | 0.010 | 0.047 | 0.154 | 0.424 |
| E184-105_F3 | I | 0.075 | 0.147 | 0.030 | 0.096 |
| A18-452.1 | IV | n.t. | n.t. | n.t. | n.t. |
| SARS2.A63-652-1.4 | I/IV | n.t. | n.t. | n.t. | n.t. |

TABLE E3A
pseudotyped neutralization potency (IC50 and IC80) for the tested anti-SARS-CoV-2
monoclonal antibodies (IC50 and IC80 are expressed in ug/ml; n.t.: not tested).

| | | Sarbecovirus Clade 1a | | | | | |
|---|---|---|---|---|---|---|---|
| | | XBB.1.5 | | EG.5.1 | | RaTG13-d21aa | |
| mAbs | Class | IC50 | IC80 | IC50 | IC80 | IC50 | IC80 |
| F769-E12 | I | 0.0068 | 0.032 | 0.138 | 0.562 | 0.048 | 0.070 |
| F770-G11 | II | 0.086 | 0.315 | 0.110 | 0.286 | 0.0063 | 0.034 |
| A80-102_G5 | I/IV | 3.5 | 8.6 | 2.0 | 2.9 | 0.083 | 0.103 |
| A18-448.1 | I/IV | 0.0039 | 0.011 | 0.0026 | 0.0100 | 0.100 | 0.922 |
| F768-104_B2 | III | 0.244 | 0.510 | 0.066 | 0.165 | 0.028 | 0.125 |
| F770-E8 | III | 0.035 | 0.171 | 0.084 | 1.056 | >10 | >10 |
| F768-D11 | I | 0.0086 | 0.021 | 0.154 | 0.424 | 0.025 | 0.072 |
| E184-105_F3 | I | 0.047 | 0.076 | 0.030 | 0.096 | 0.014 | 0.039 |
| A18-452.1 | IV | 1.79 | 2.22 | n.t. | n.t. | n.t. | n.t. |
| SARS2.A63-652-1.4 | I/IV | 0.0080 | 0.015 | n.t. | n.t. | n.t. | n.t. |
| | | Sarbecovirus Clade 1a | | | | | |
| | | Pangolin-GD | | Pangolin_GX-PSL | | Pangolin_GX-P2V | |
| mAbs | Class | IC50 | IC80 | IC50 | IC80 | IC50 | IC80 |
| F769-E12 | I | 0.0061 | 0.012 | 0.013 | 0.051 | 0.0082 | 0.024 |
| F770-G11 | II | 0.0091 | 0.017 | 2.0 | 7.0 | 2.6 | 7.2 |
| A80-102_G5 | I/IV | 0.0067 | 0.019 | 0.0075 | 0.083 | 0.061 | 0.091 |
| A18-448.1 | I/IV | 0.0010 | 0.0016 | >10 | >10 | >10 | >10 |
| F768-104_B2 | III | 0.031 | 0.076 | >10 | >10 | 9.6 | >10 |
| F770-E8 | III | 0.0032 | 0.0067 | >10 | >10 | >10 | >10 |
| F768-D11 | I | 0.0090 | 0.020 | 0.0051 | 0.011 | 0.0021 | 0.0049 |
| E184-105_F3 | I | 0.0043 | 0.0078 | 0.0056 | 0.014 | 0.0065 | 0.014 |
| A18-452.1 | IV | n.t. | n.t. | 0.026 | 0.381 | 0.248 | 1.2 |
| SARS2.A63-652-1.4 | I/IV | n.t. | n.t. | 0.0080 | 0.016 | 0.0050 | 0.0090 |
| | | Sarbecovirus Clade 1b | | | | | |
| | | SARS | | WIV1 | | SHC014 | |
| mAbs | Class | IC50 | IC80 | IC50 | IC80 | IC50 | IC80 |
| F769-E12 | I | >10 | >10 | >10 | >10 | >10 | >10 |
| F770-G11 | II | >10 | >10 | >10 | >10 | >10 | >10 |
| A80-102_G5 | I/IV | 0.027 | 0.085 | 0.0073 | 0.040 | 0.0068 | 0.036 |
| A18-448.1 | I/IV | 0.0049 | 0.016 | 0.0022 | 0.012 | 0.0051 | 0.013 |
| F768-104_B2 | III | 0.125 | 0.339 | 10.952 | >10 | >10 | >10 |
| F770-E8 | III | >10 | >10 | >10 | >10 | >10 | >10 |
| F768-D11 | I | >10 | >10 | >10 | >10 | >10 | >10 |
| E184-105_F3 | I | >10 | >10 | >10 | >10 | >10 | >10 |
| A18-452.1 | IV | 0.071 | 0.308 | 0.042 | >50 | 0.064 | 0.457 |
| SARS2.A63-652-1.4 | I/IV | >50 | >50 | >50 | >50 | >50 | >50 |

TABLE E3B
pseudotyped neutralization potency (IC50 and IC80) for the tested anti-SARS-CoV-2
monoclonal antibodies (IC50 and IC80 are expressed in nM; n.t. = not tested).

| | | BA.2.86 | | EG.5.1 | | XBB.1.16.6 | | FL.1.5.1 | | HV.1 | |
|---|---|---|---|---|---|---|---|---|---|---|---|
| | | IC50 | IC80 | IC50 | IC80 | IC50 | IC80 | IC50 | IC80 | IC50 | IC80 |
| mAbs | F729-E12 | 0.0816 | 0.3672 | 1.3623 | 2.7333 | 2.3194 | 6.5314 | 1.2147 | 2.3973 | 1.7517 | 5.0378 |
| | F770-G11 | >66.7 | >66.7 | 0.5393 | 1.6607 | 0.9549 | 2.4525 | 0.4259 | 1.7817 | 0.4669 | 1.9743 |
| | F770-E8 | >66.7 | >66.7 | 0.6078 | 2.2409 | 0.5781 | 5.657 | 0.7095 | 3.4474 | 0.5431 | 2.1784 |
| | F768-104_B2 | 0.5931 | 1.4237 | 0.5074 | 1.4069 | 0.9102 | 2.4031 | 0.7179 | 1.9477 | 0.7734 | 1.8961 |
| | A18-448.1 | 0.0355 | 0.1007 | 0.0215 | 0.0527 | 0.0232 | 0.0694 | 0.0196 | 0.0587 | 0.0199 | 0.0657 |

| | | HK.3 | | JN.1 | | JD.1.1 | | SARS | |
|---|---|---|---|---|---|---|---|---|---|
| | | IC50 | IC80 | IC50 | IC80 | IC50 | IC80 | IC50 | IC80 |
| mAbs | F729-E12 | 3.1117 | 7.5053 | 2.0514 | 5.5979 | 30.67 | >66.7 | >66.7 | >66.7 |
| | F770-G11 | 1.1365 | 2.1096 | >66.7 | >66.7 | 0.5067 | 1.7131 | >66.7 | >66.7 |
| | F770-E8 | 1.3306 | 9.8044 | n.t. | n.t. | n.t. | n.t. | >66.7 | >66.7 |
| | F768-104_B2 | 1.2756 | 2.4173 | 0.4655 | 0.8315 | 0.5041 | 1.1714 | 0.8382 | 2.2718 |
| | A18-448.1 | 0.0194 | 0.0724 | 0.0367 | 0.128 | 0.0194 | 0.0779 | 0.0328 | 0.11 |

### Example 2

### Design of Antigen Binding Polypeptides and Antigen Binding Polypeptide Complexes

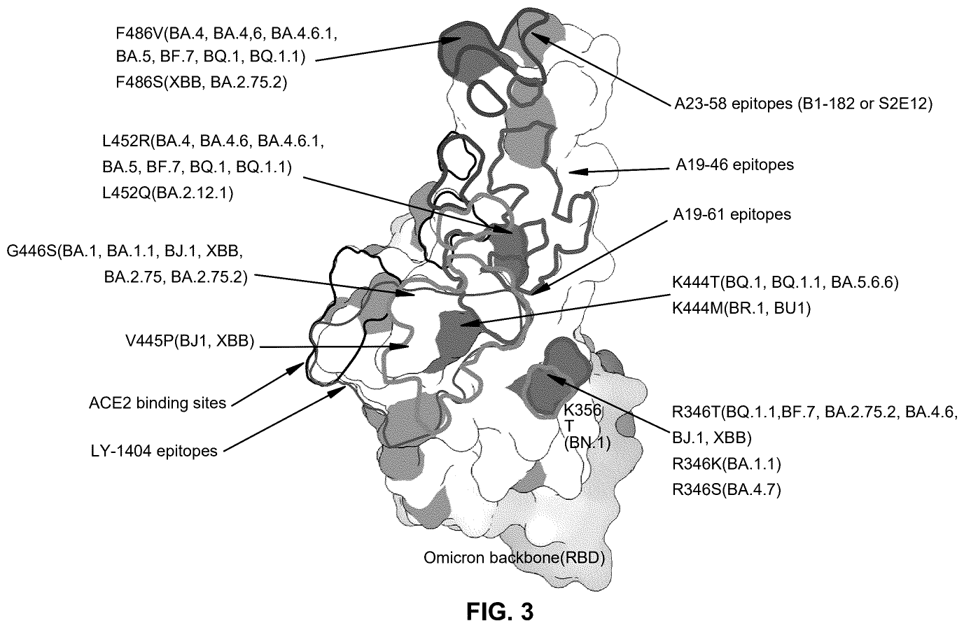

The antigen binding polypeptides described herein were designed to comprise different combinations of heavy chain variable regions (VH) and light chain variable regions (VL) derived from the nine different anti-SARS-CoV-2 neutralizing antibodies selected as described in Example 1. The nine anti-SARS-CoV-2 neutralizing antibodies (B8, E12, E8, A23-58, B1, G11, B2, A18-448, and A19-46)) used herein, can be described as Class I, II, III, or IV anti-SARS-CoV-2 neutralizing antibodies, and target multiple spike protein receptor binding domain (RBD) sites (see, e.g., FIG. 3). Additionally, an anti-SARS-CoV-2 neutralizing antibody disclosed herein can also display properties of more than one Classes of anti-SARS-CoV-2 antibodies. For example, an antibody (e.g., A18-448) can bind to an epitope to which antibodies belonging to one of the four anti-SARS-CoV-2 antibody Classes bind and can compete for binding with one or more antibodies belonging to one or more different Classes of anti-SARS-CoV-2 antibodies. For example, A18-448 binds to an epitope located in a region of RBD to which Class IV anti-SARS-CoV-2 antibodies bind, but can compete for binding to RBD with anti-SARS-CoV-2 antibodies belonging to Class I and Class III. Thus, A18-448 can be described, for example, as a Class I/IV anti-SARS-CoV-2 antibody.

The antigen binding polypeptides described herein can comprise one VL and one VH, or can comprise more than one VLs and more than VHs.

The antigen binding polypeptides described herein were designed to be assembled in antigen binding polypeptide complexes, wherein each antigen binding polypeptide complex comprises two antigen binding polypeptides. The two antigen binding polypeptides comprised in each antigen binding polypeptide complex can be the same or can be different. The antigen binding polypeptide complexes described herein were designed to comprise (a) VLs and VHs derived from one of the six anti-SARS-CoV-2 neutralizing antibodies (i.e., are mono-specific). (b) VLs and VHs derived from two of the six anti-SARS-CoV-2 neutralizing antibodies (i.e., are bi-specific). (c) VLs and VHs derived from three of the six anti-SARS-CoV-2 neutralizing antibodies (i.e., are tri-specific), or (d) VLs and VHs derived from four of the six anti-SARS-CoV-2 neutralizing antibodies (i.e., are tetra-specific).

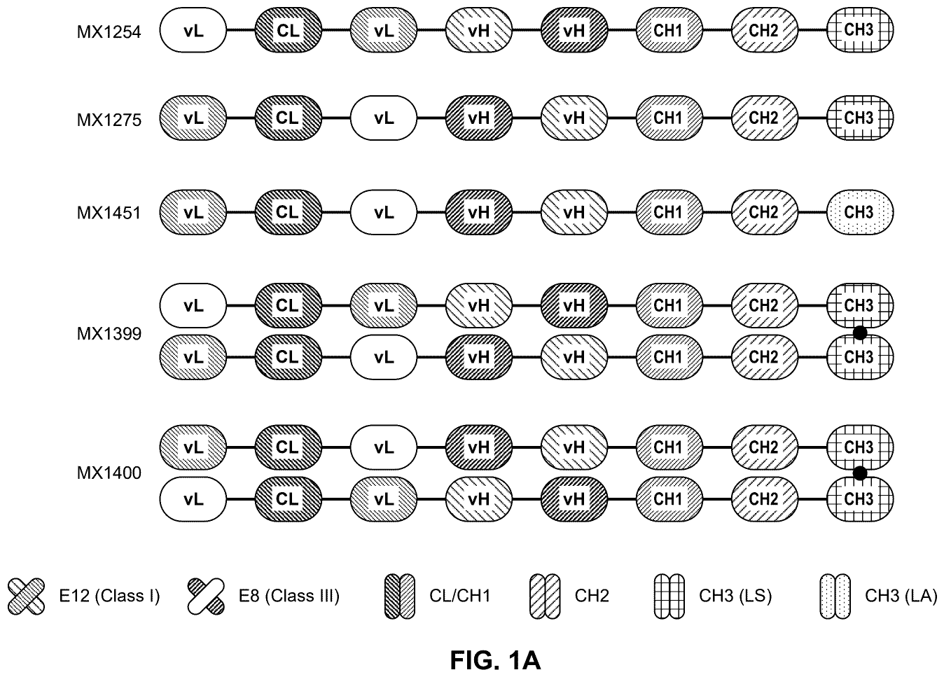

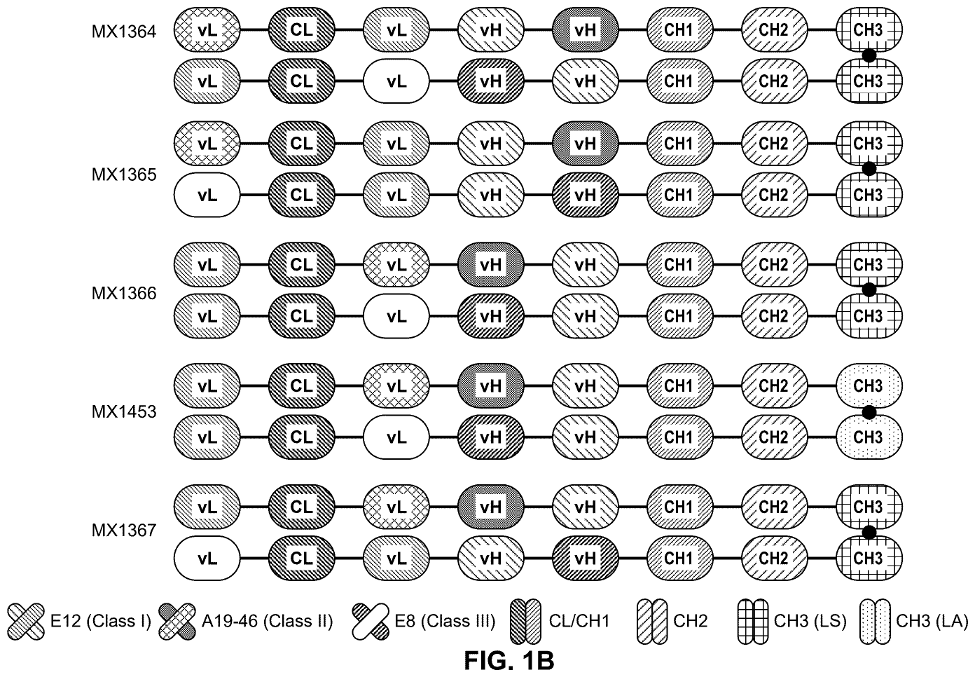

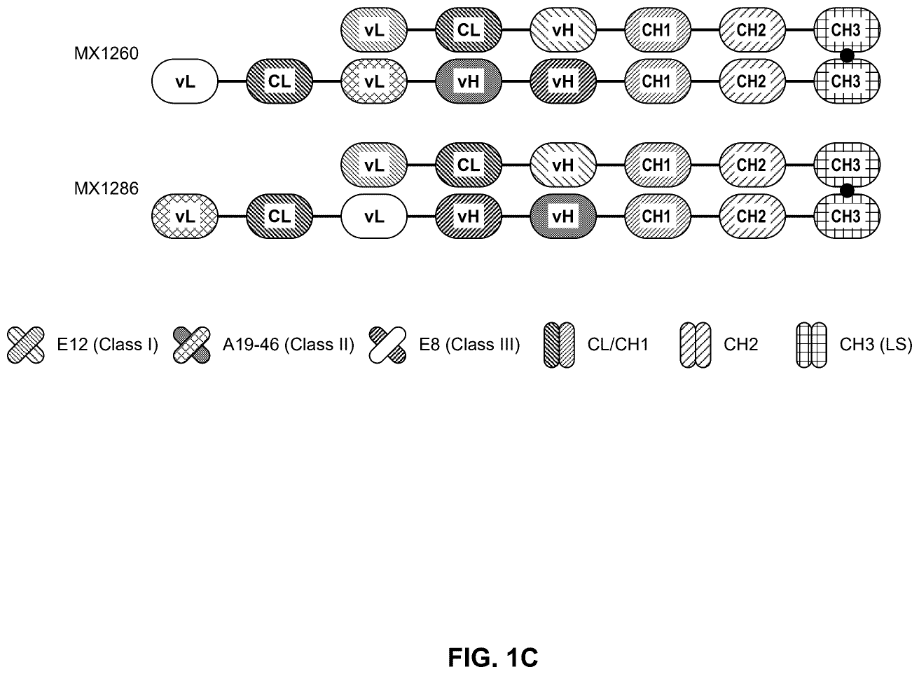

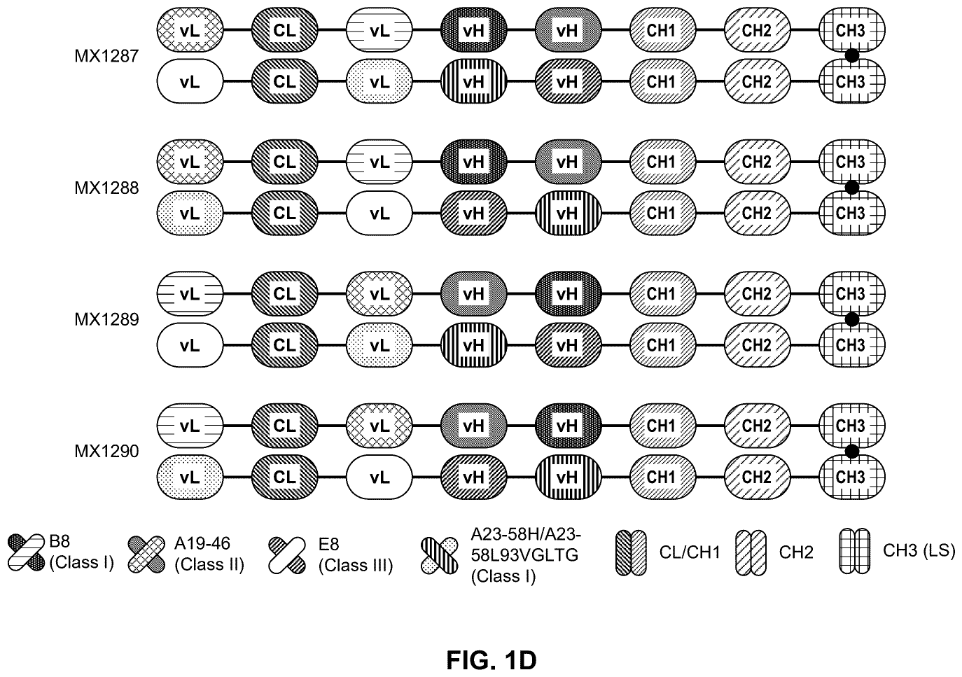

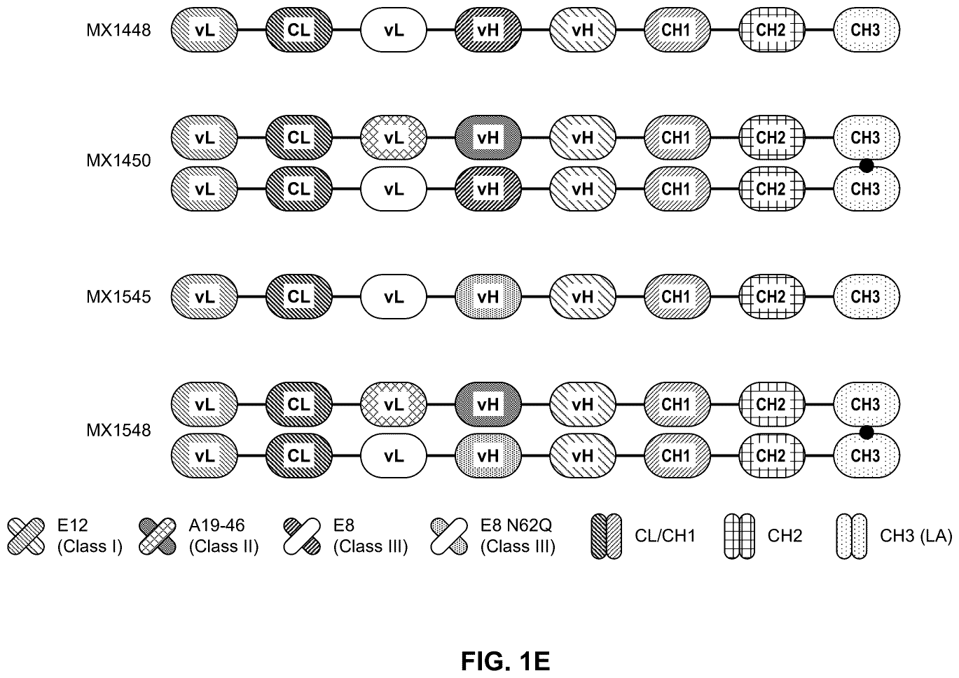

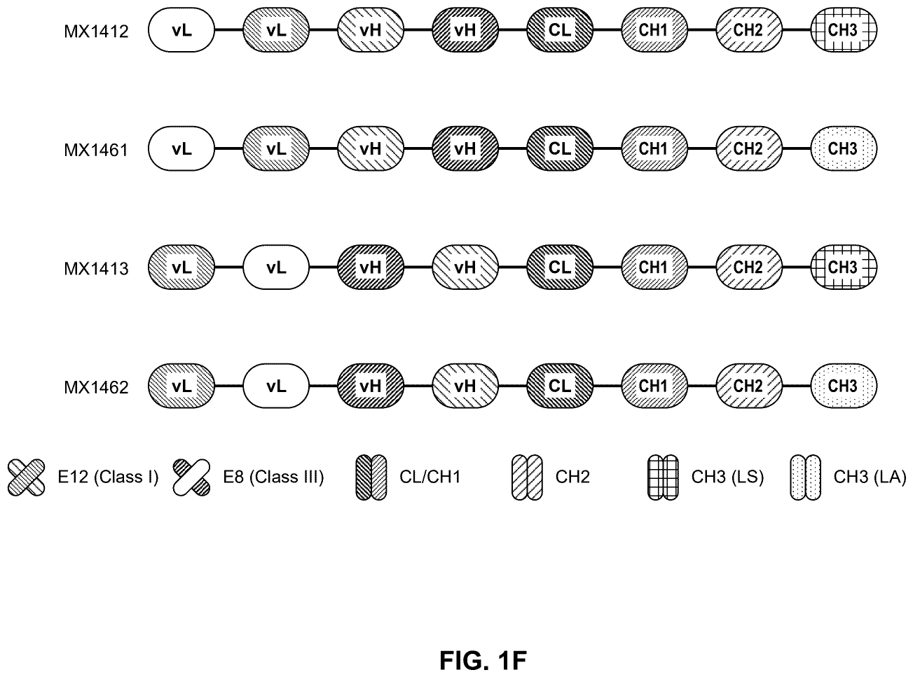

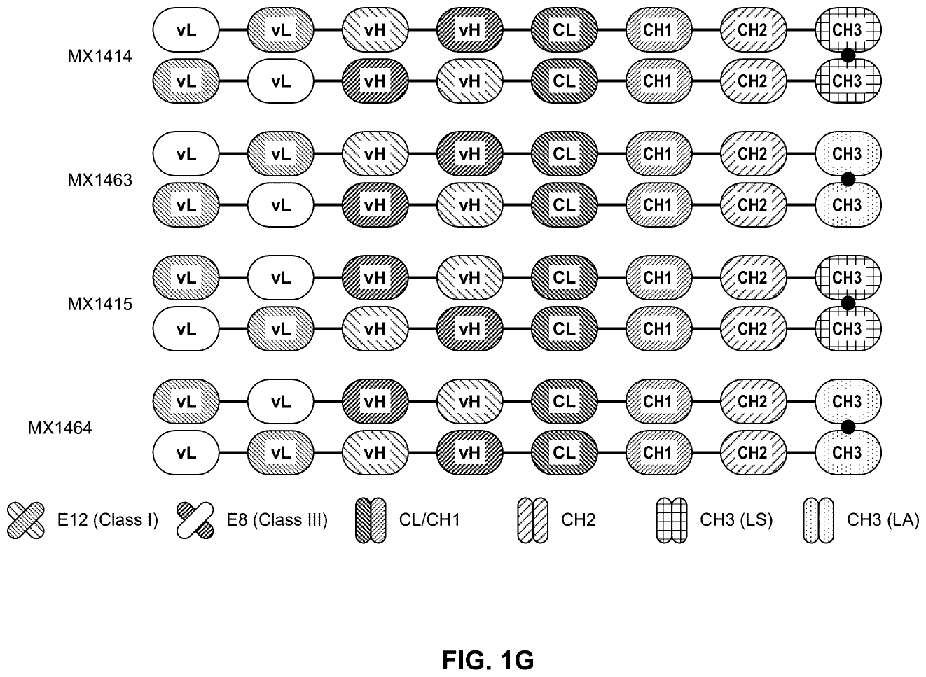

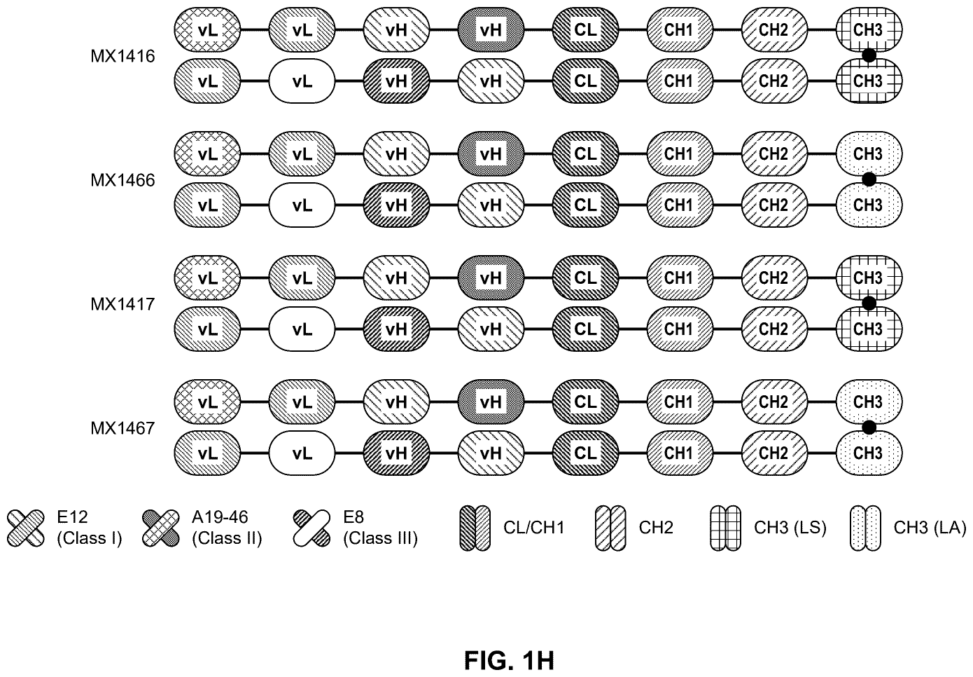

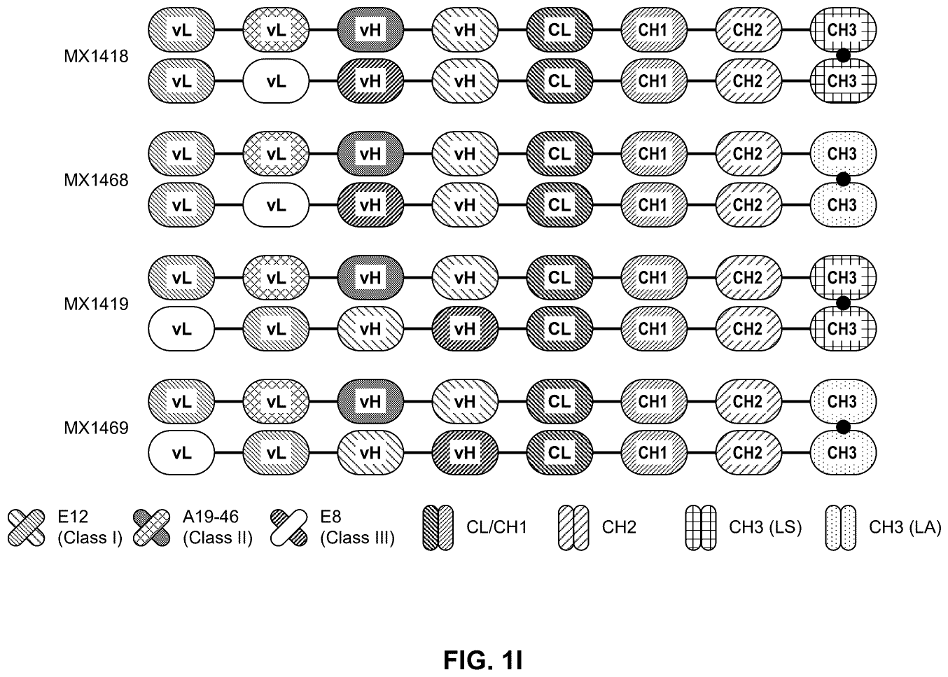

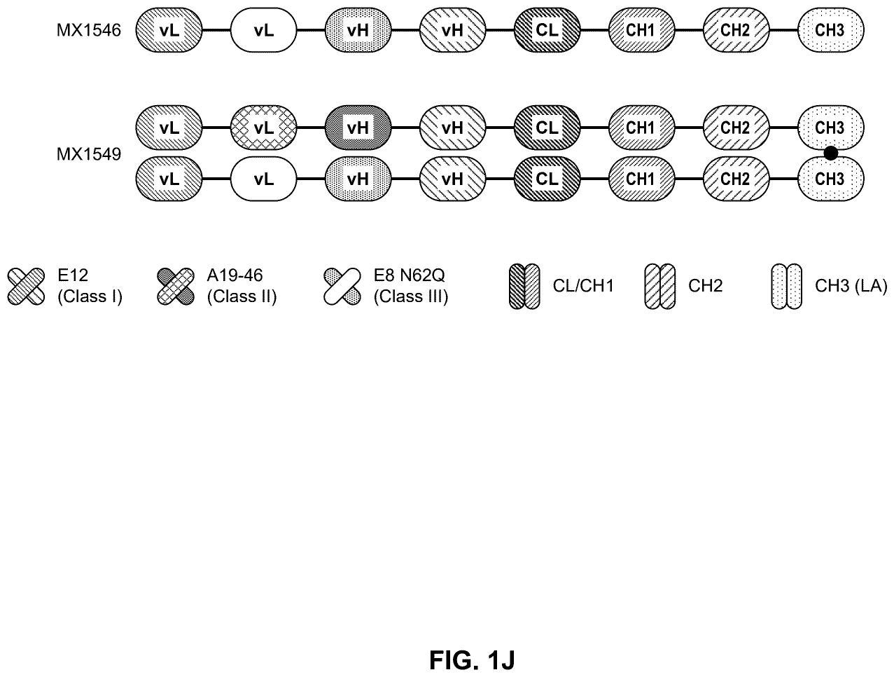

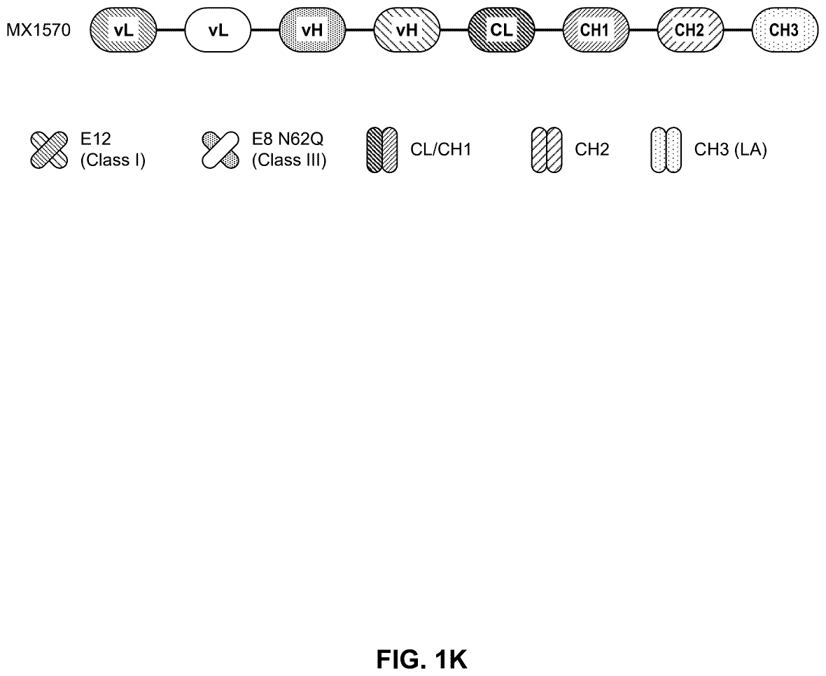

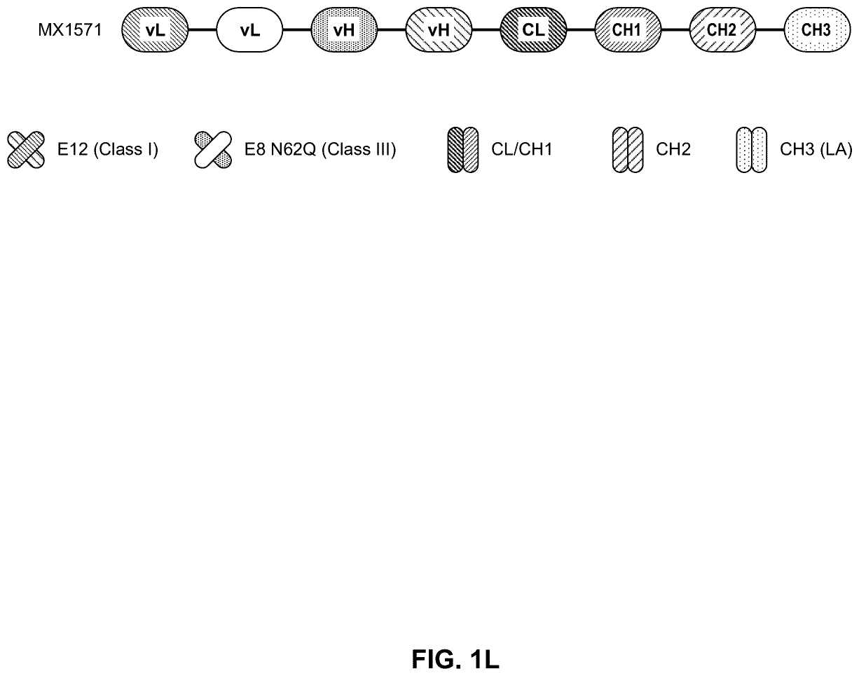

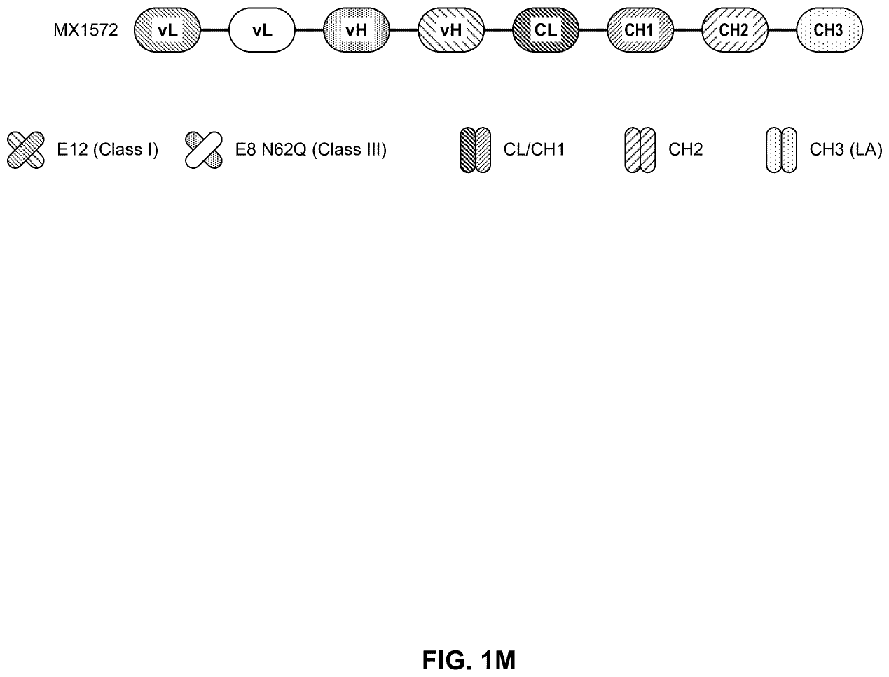

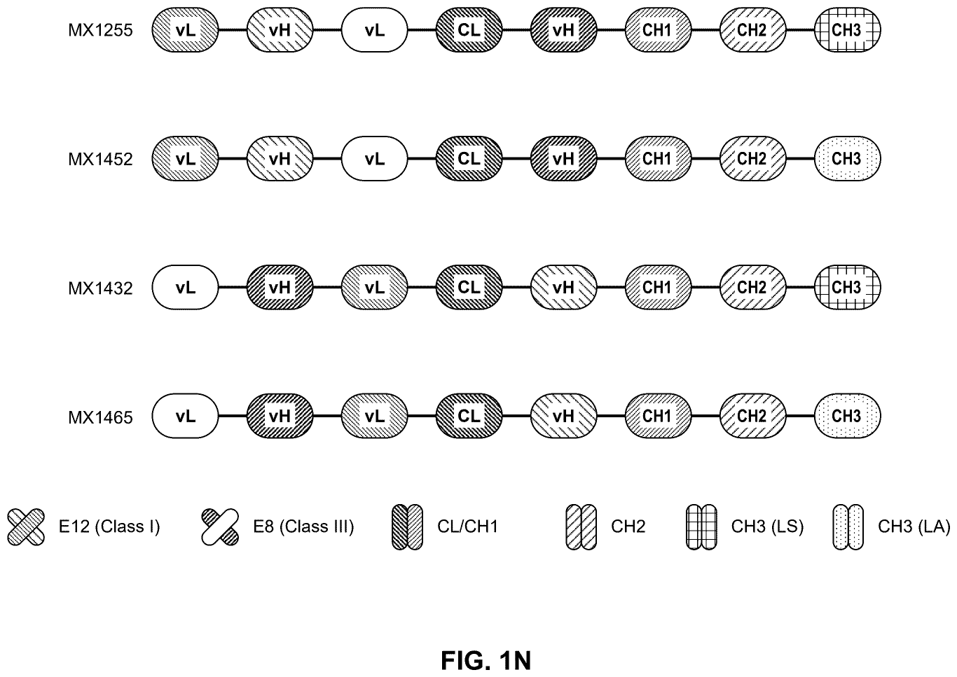

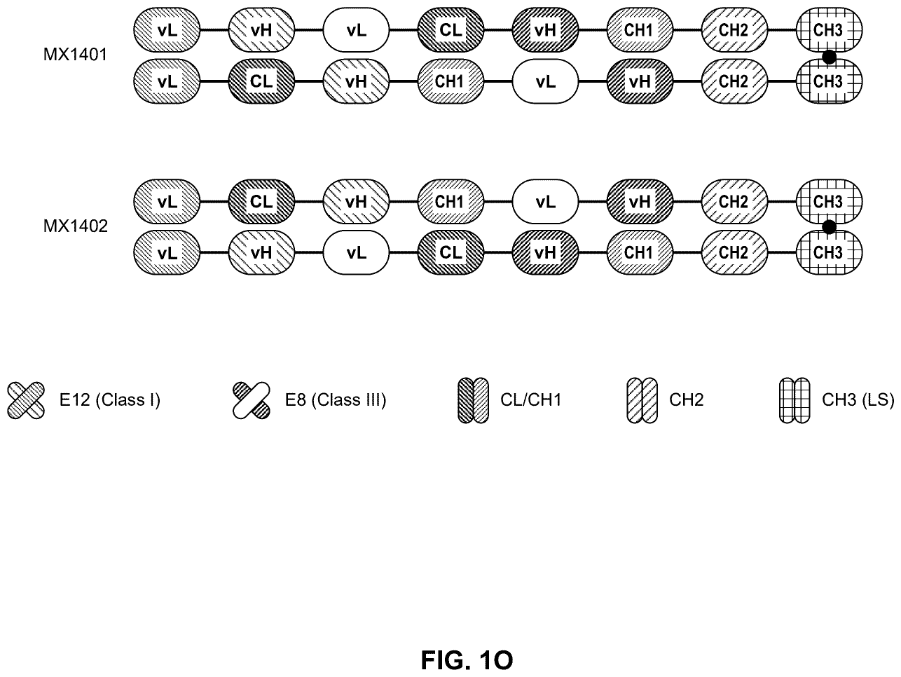

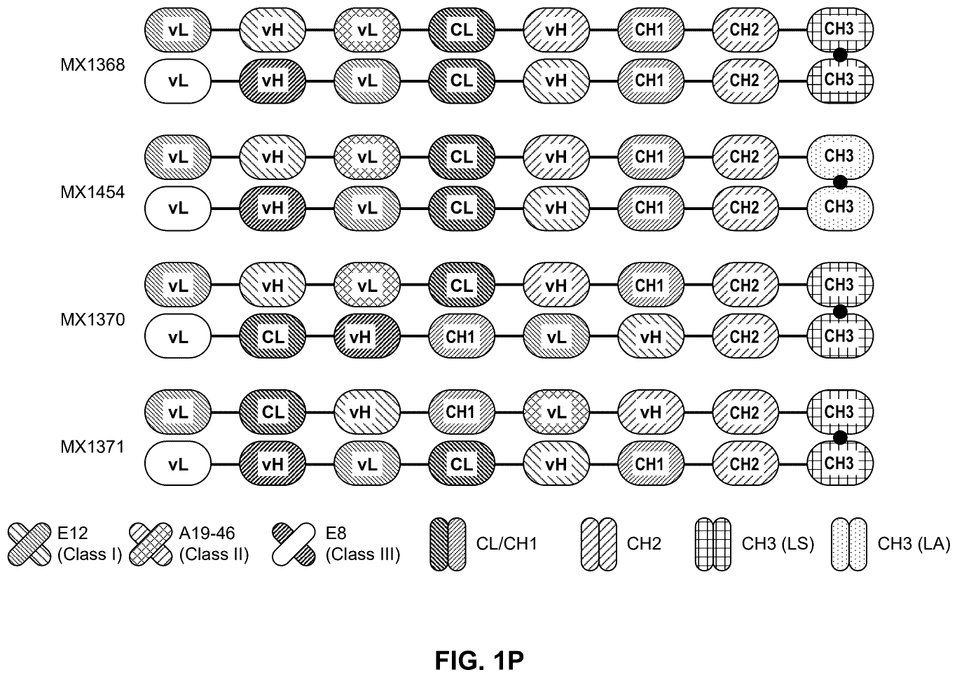

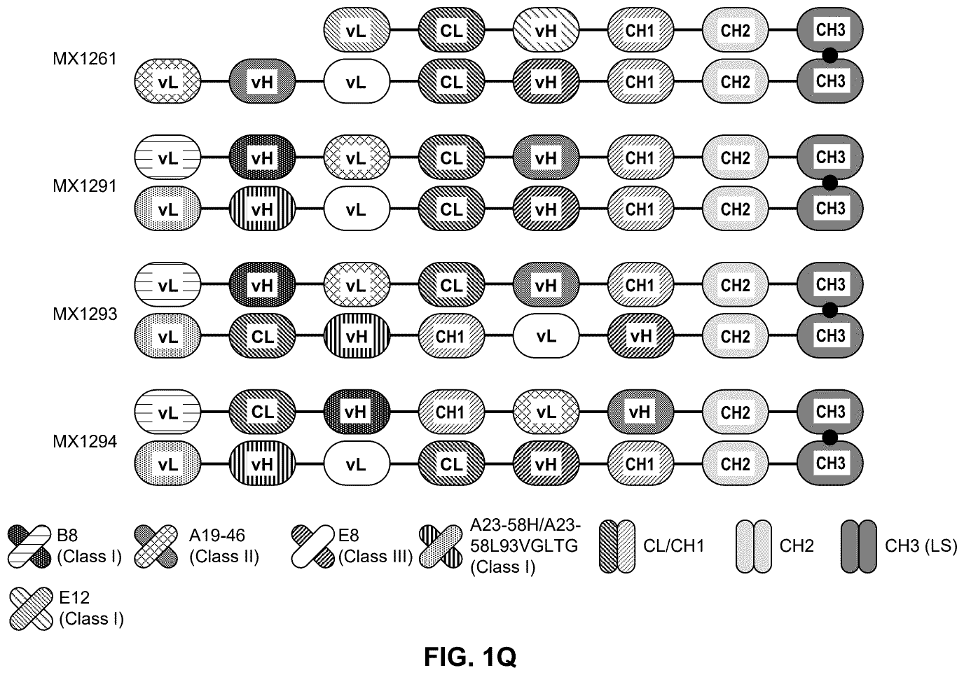

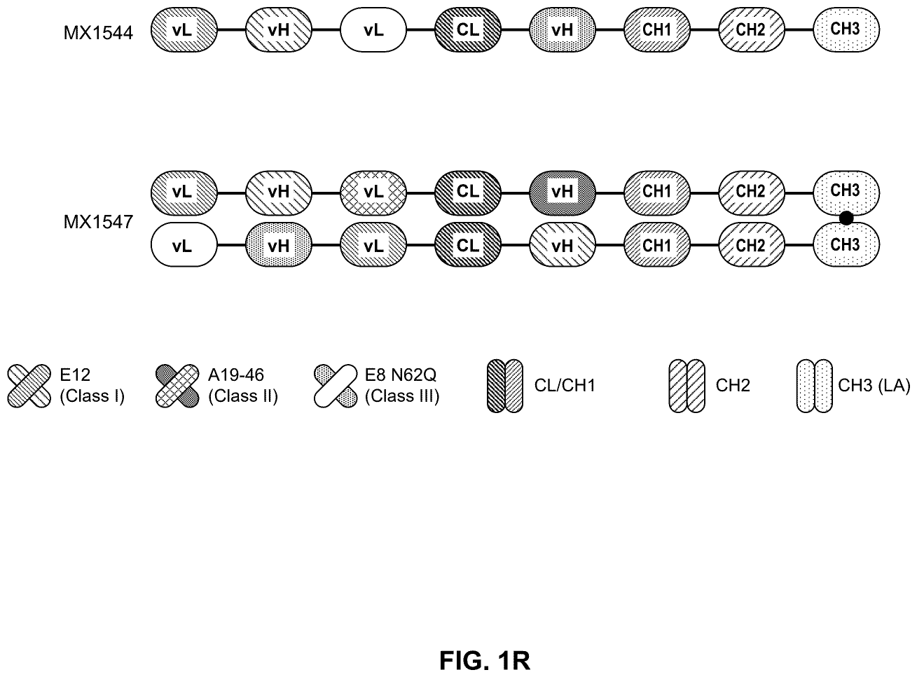

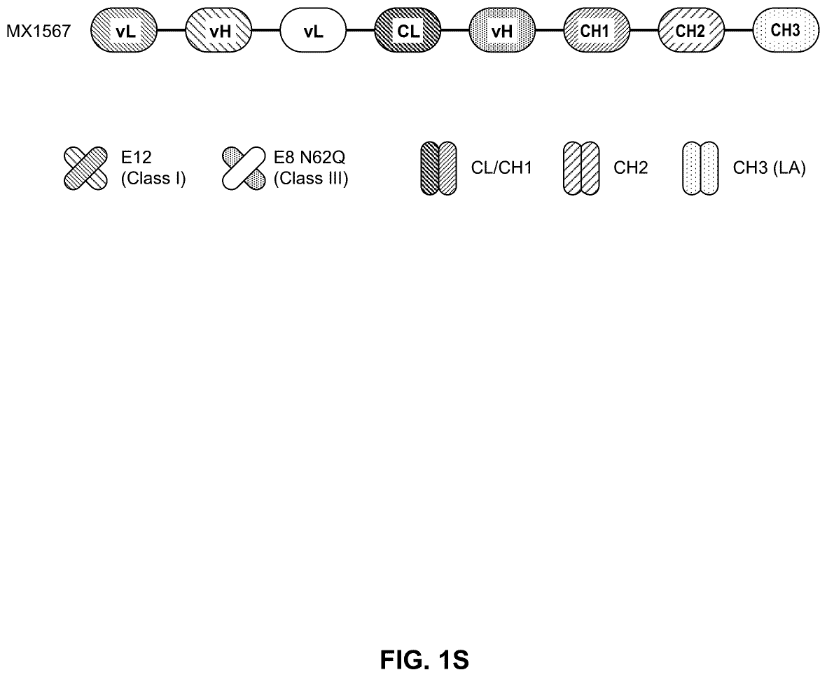

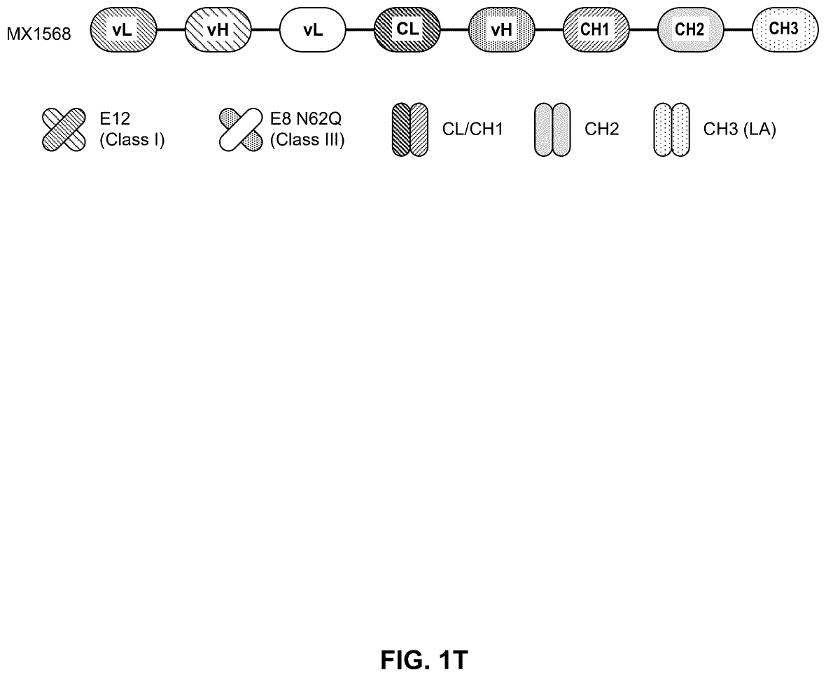

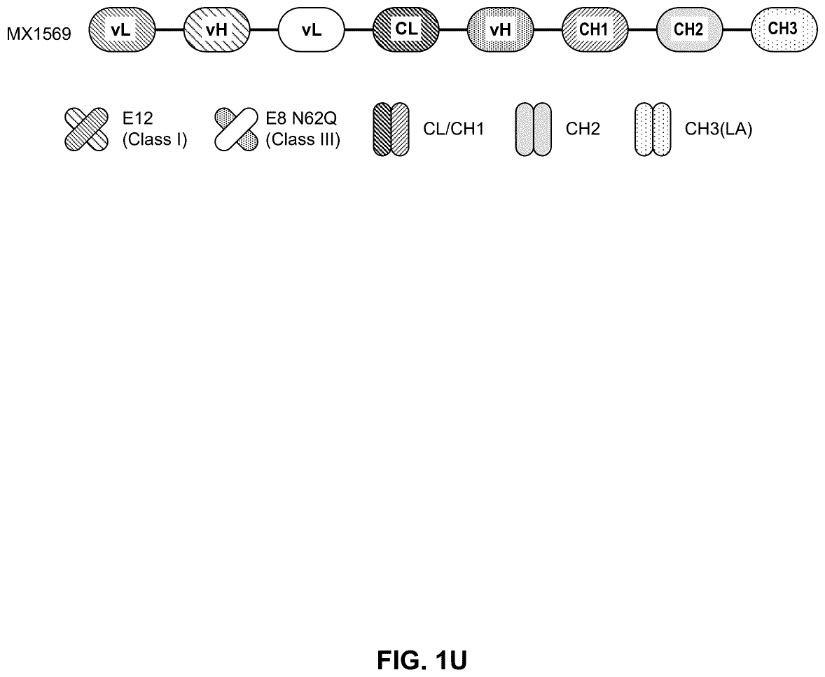

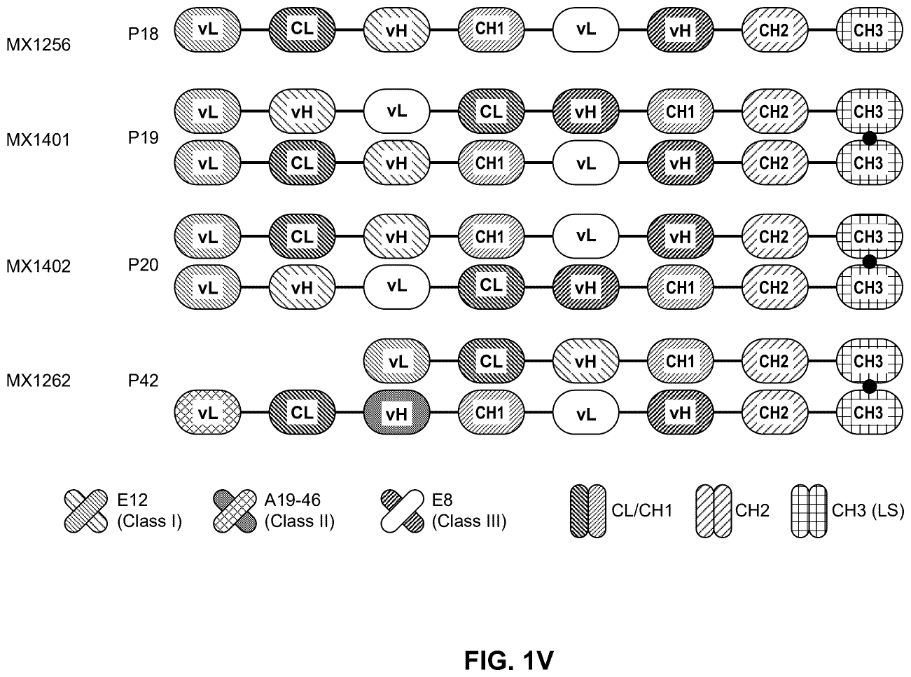

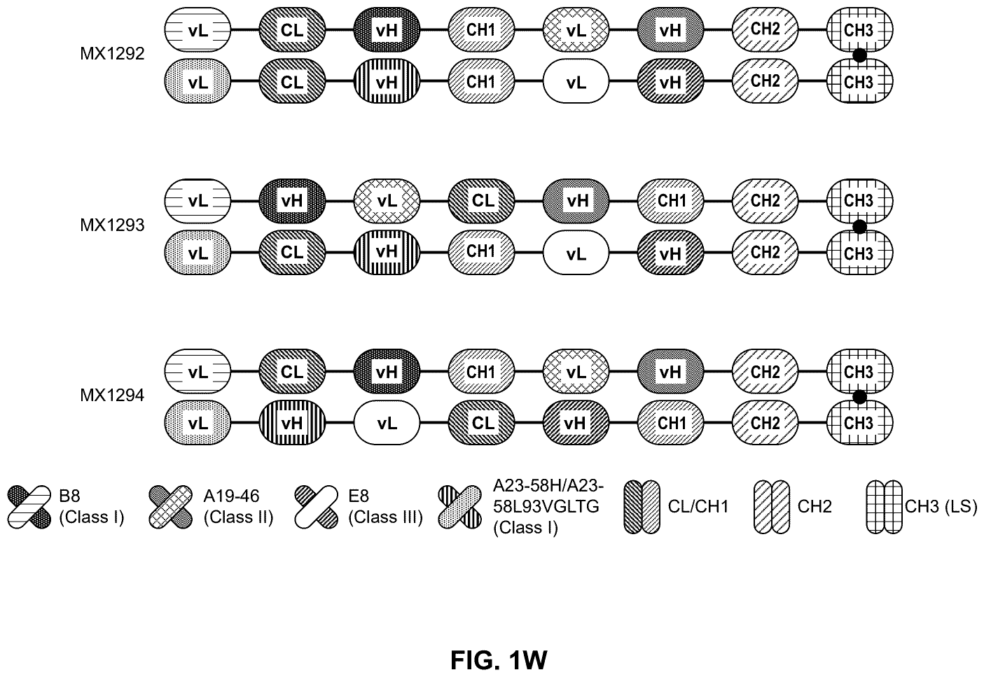

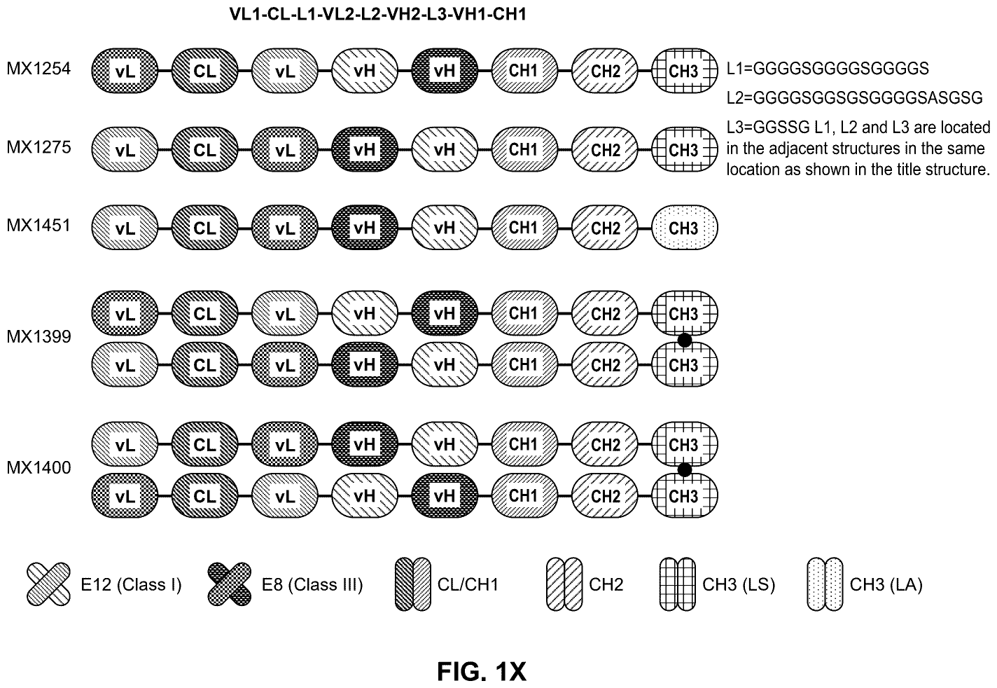

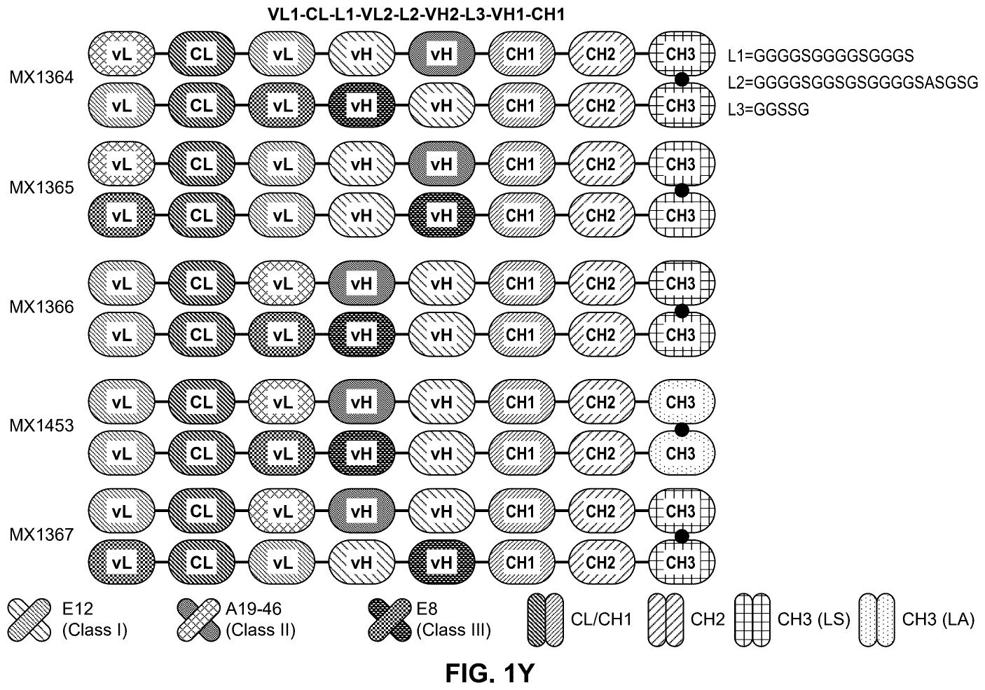

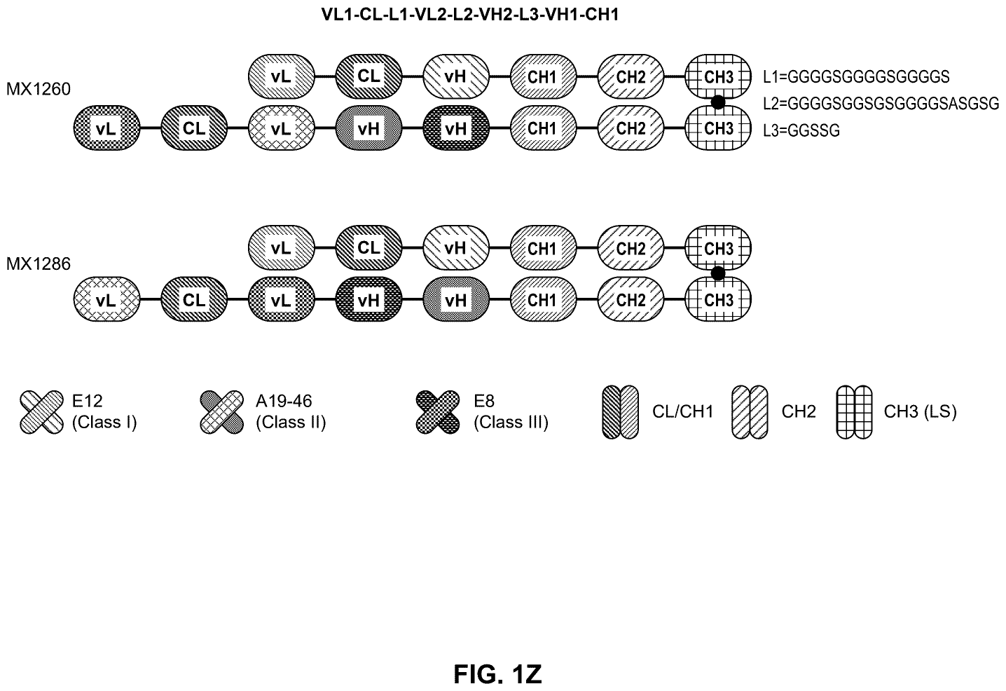

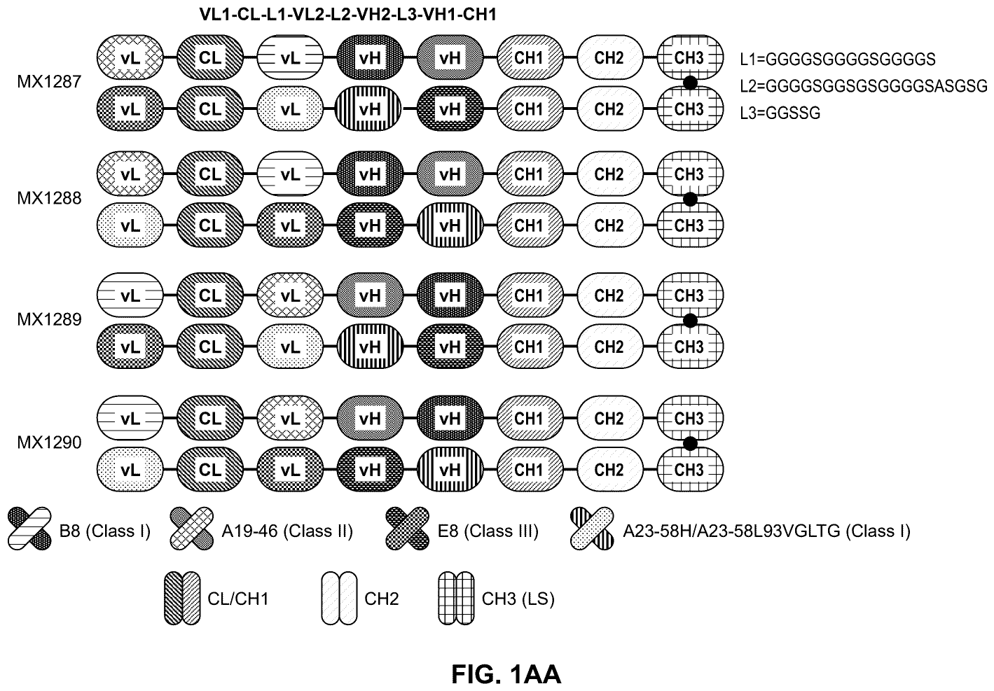

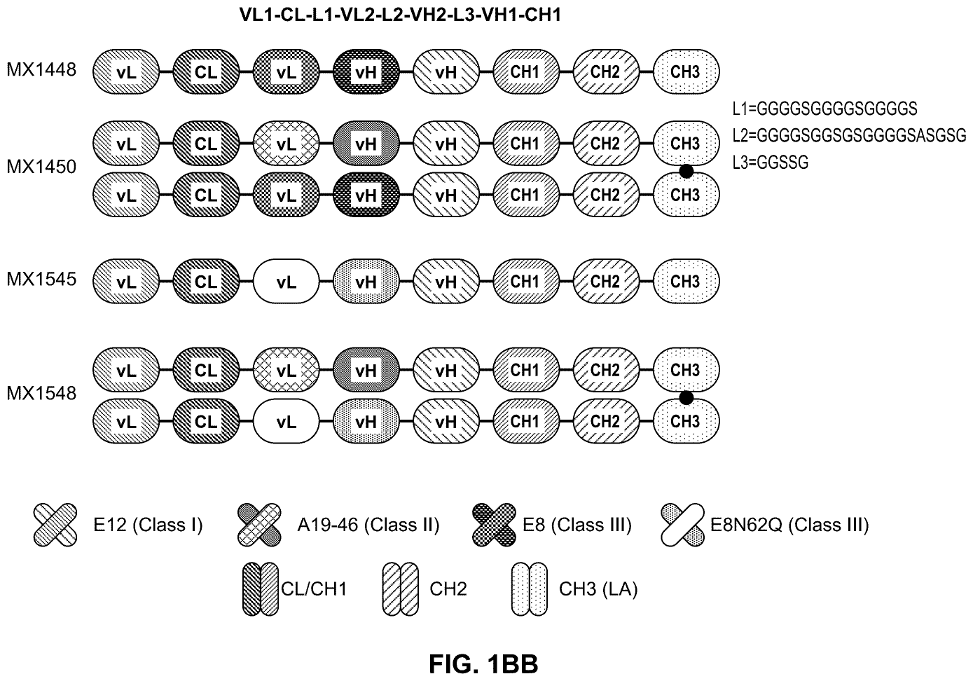

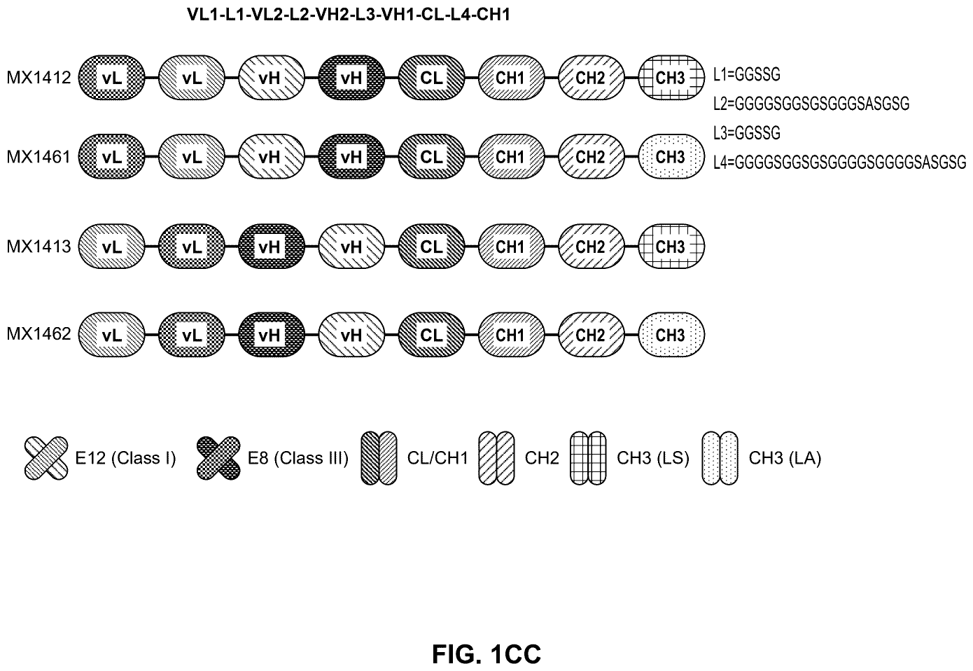

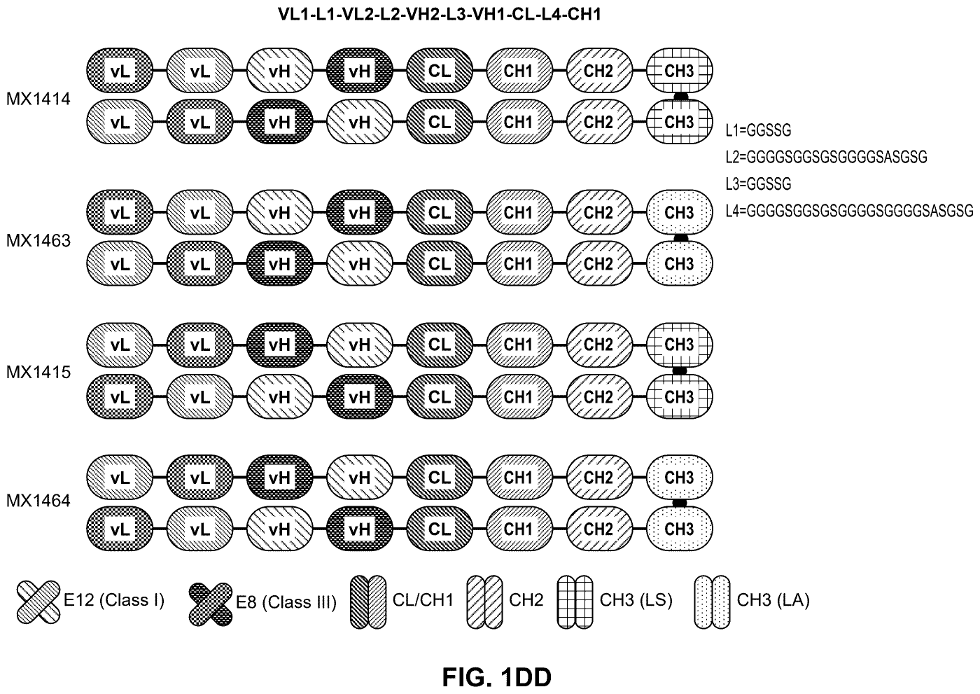

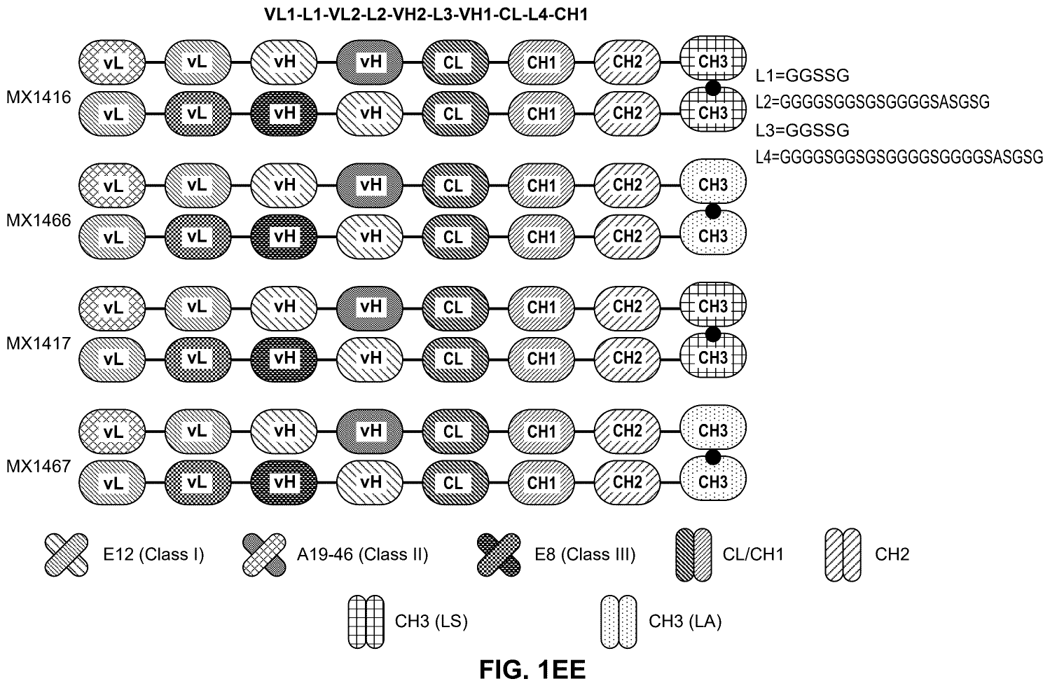

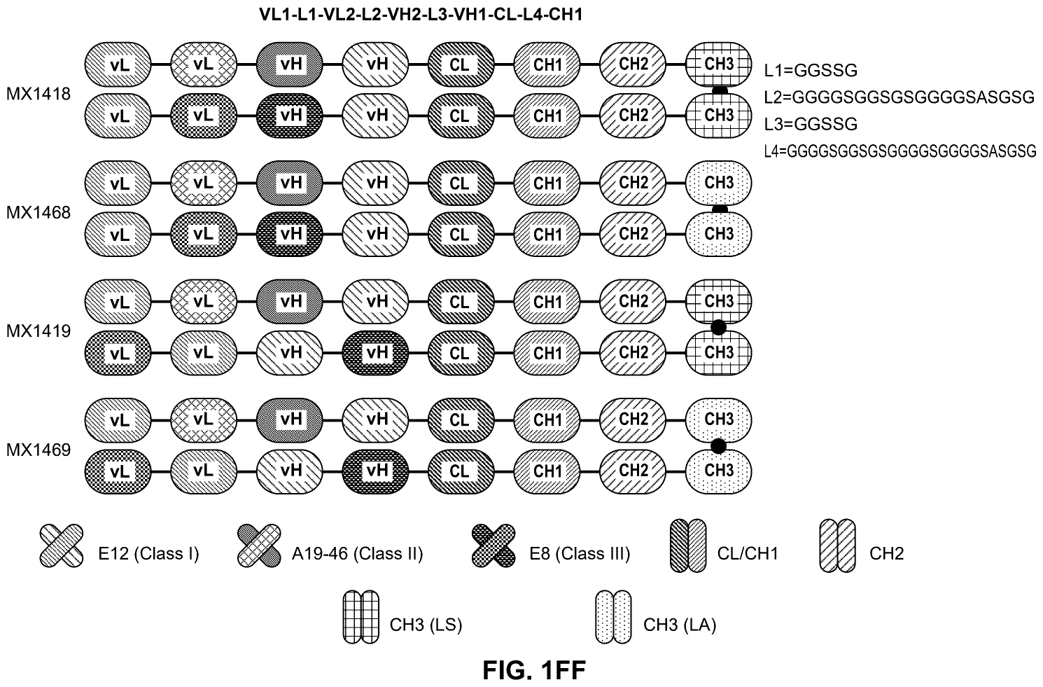

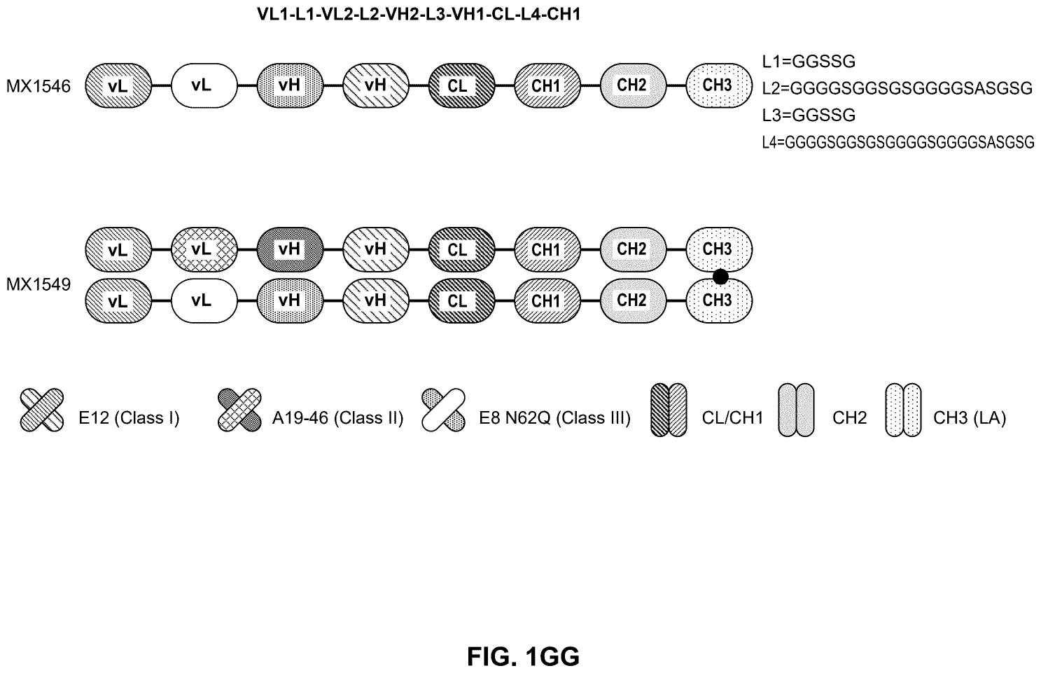

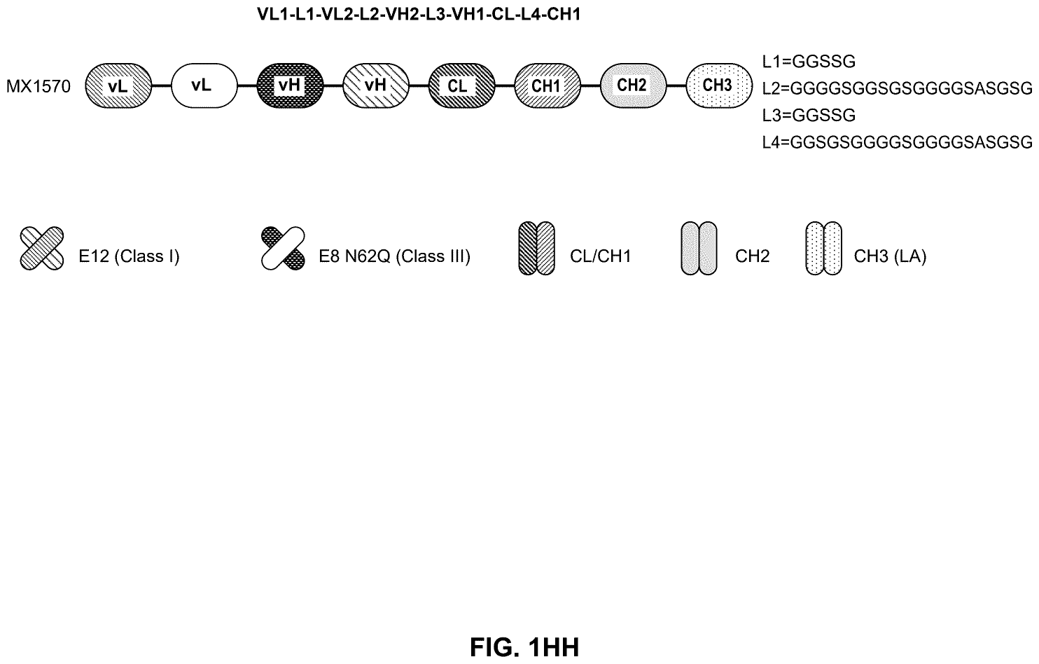

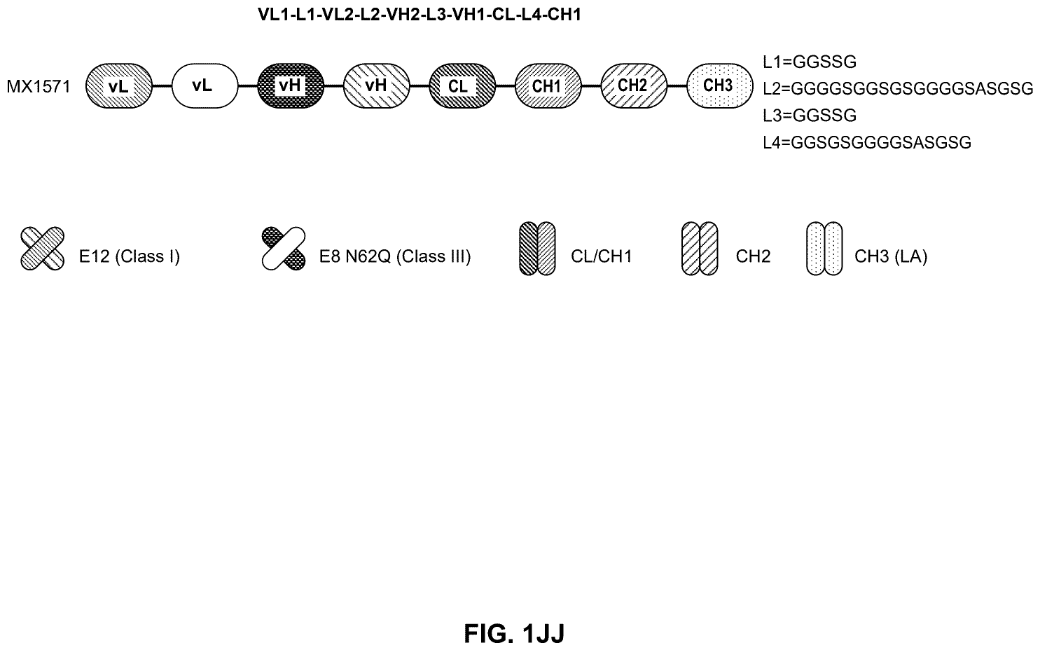

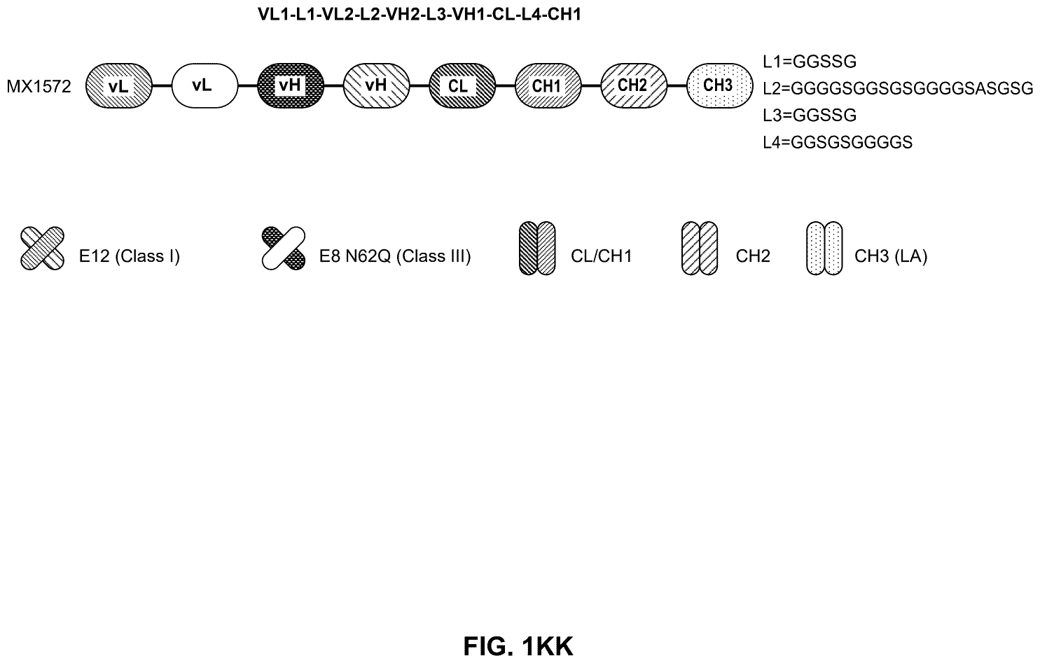

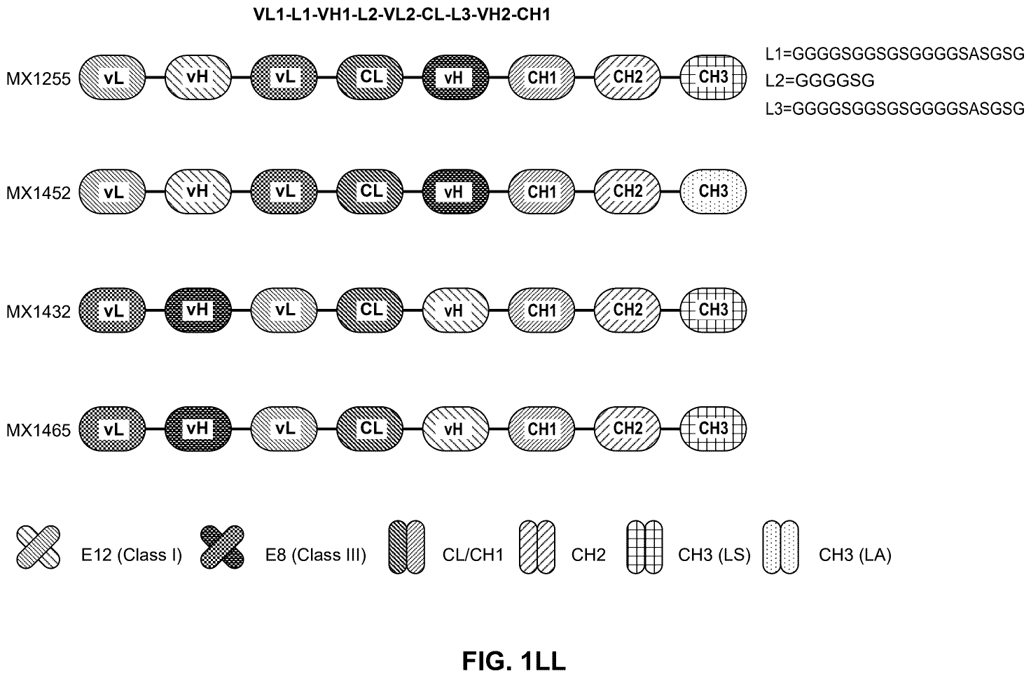

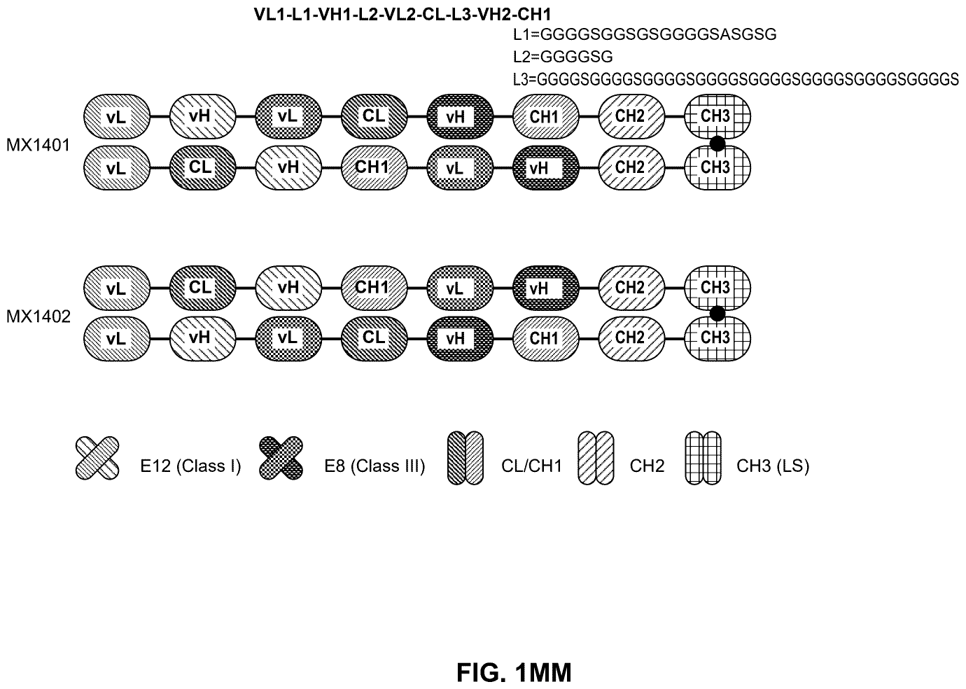

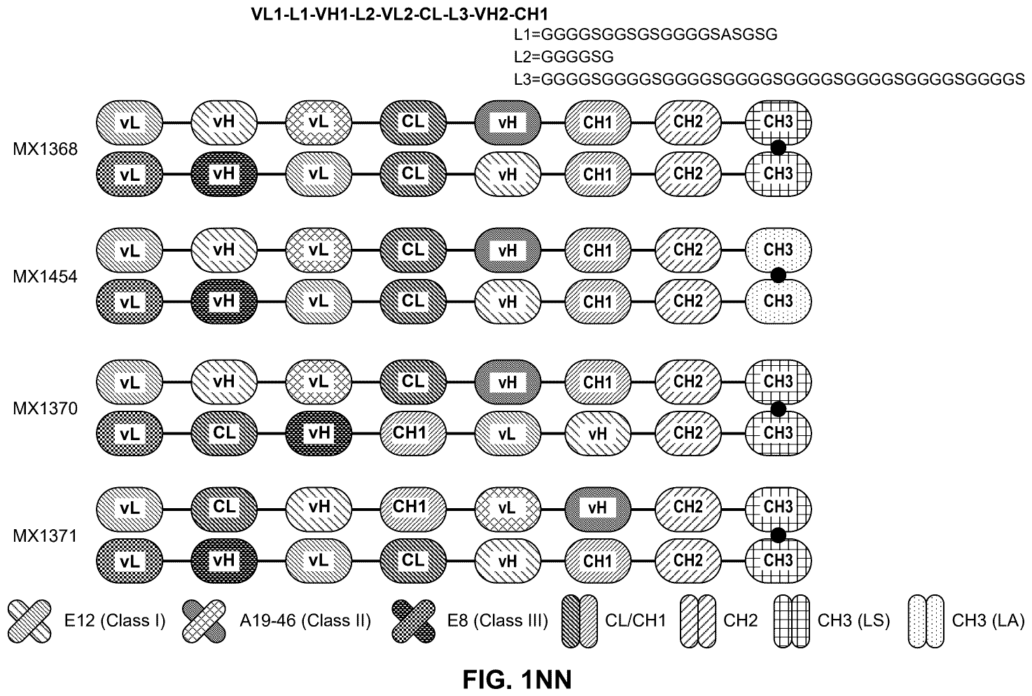

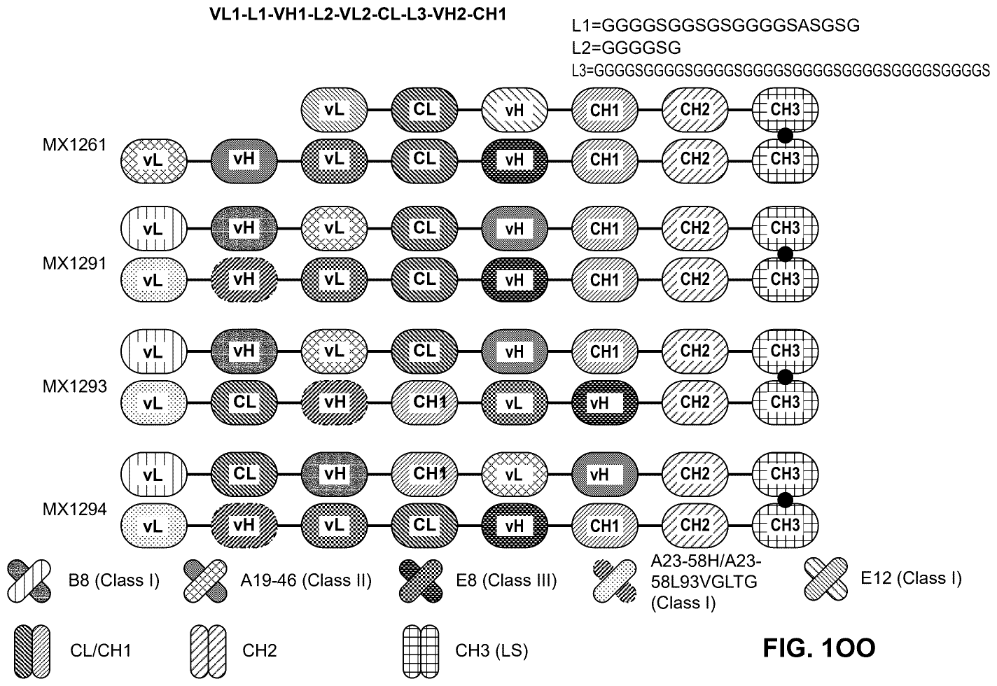

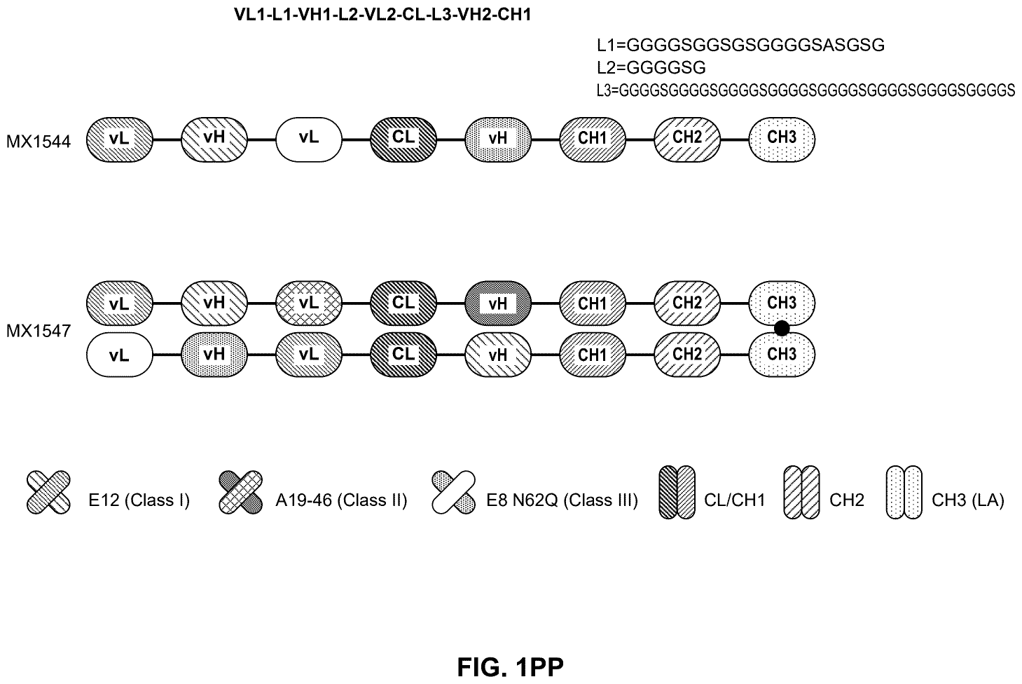

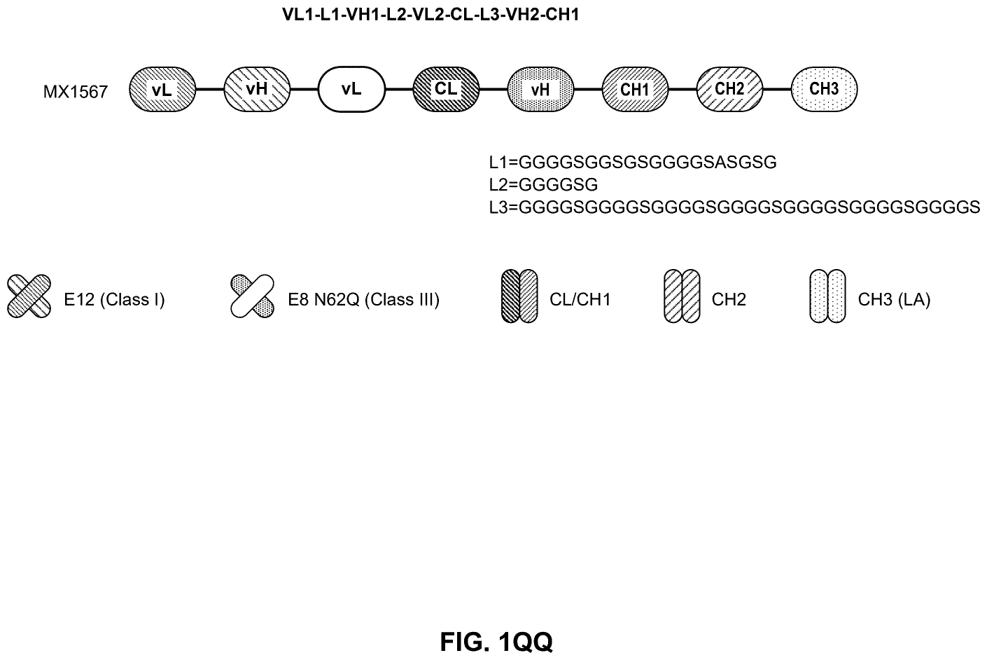

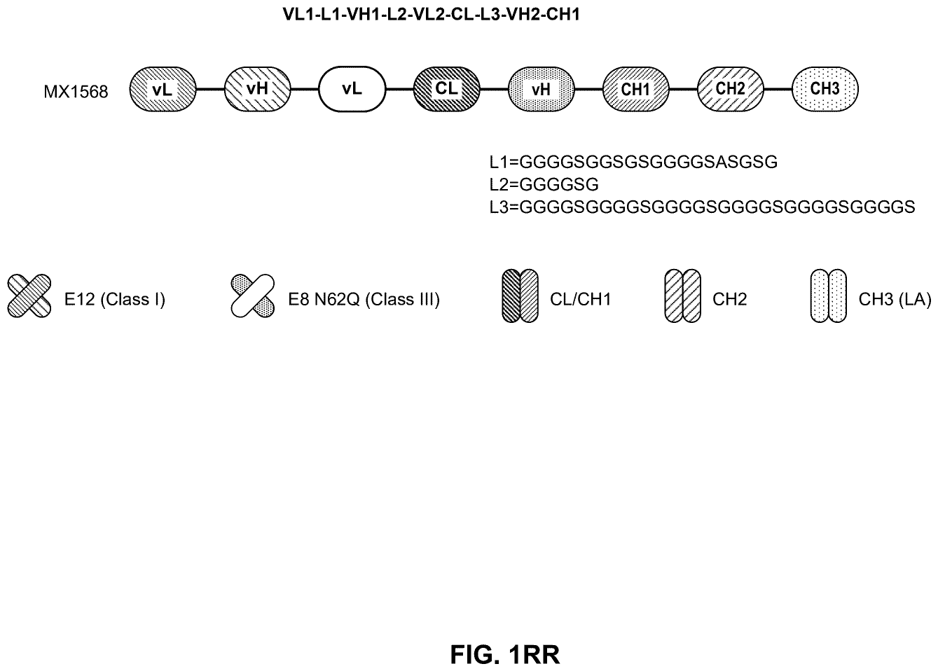

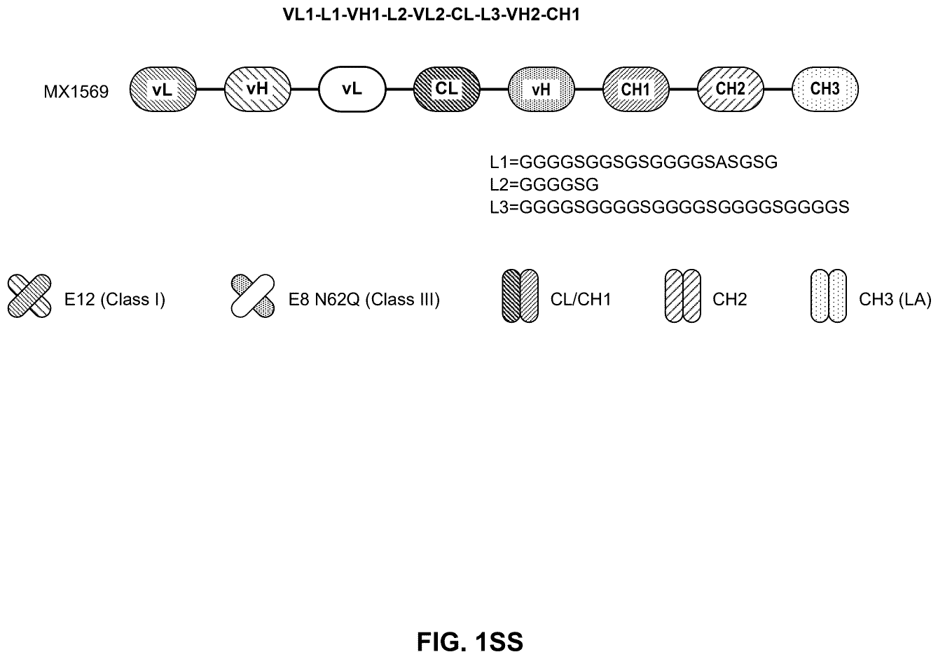

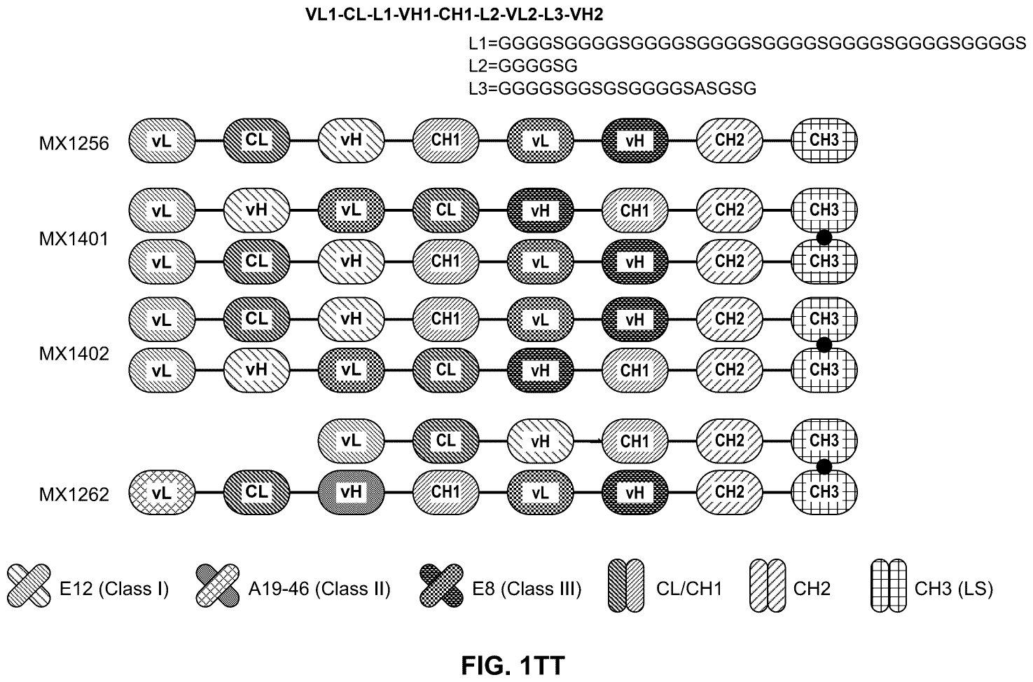

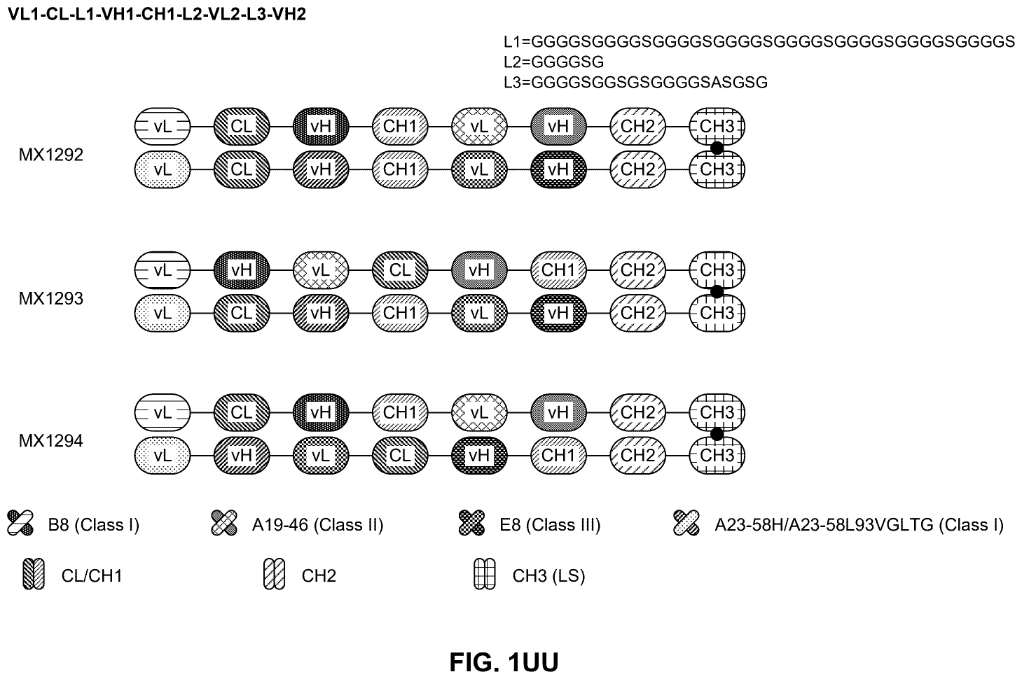

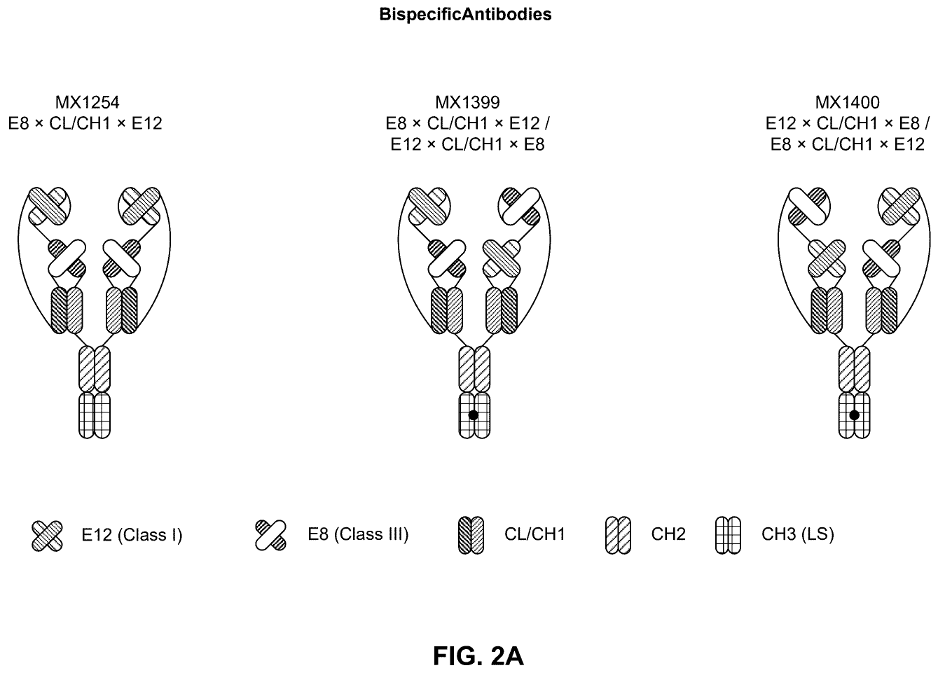

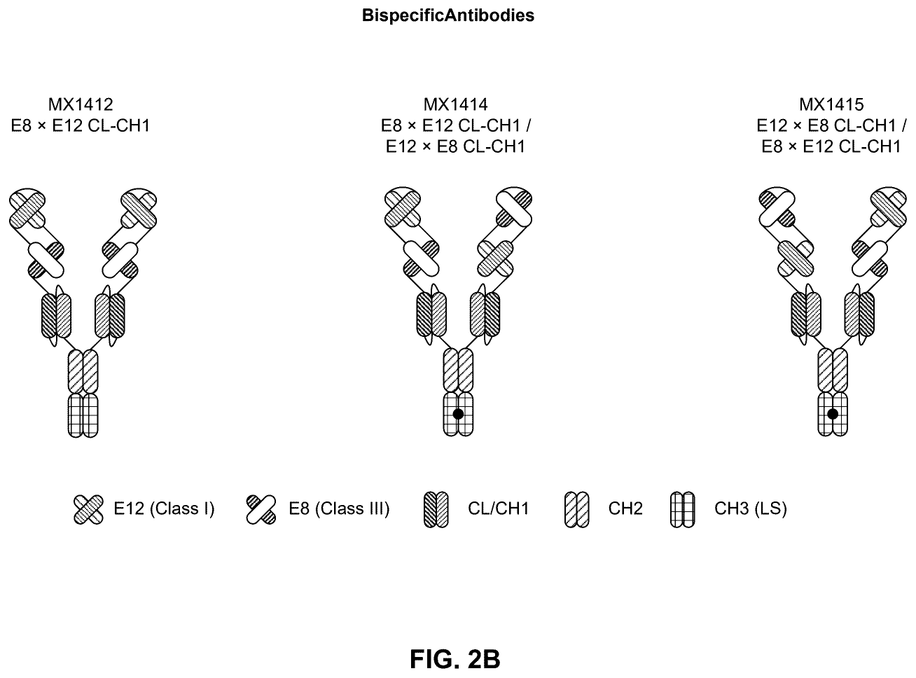

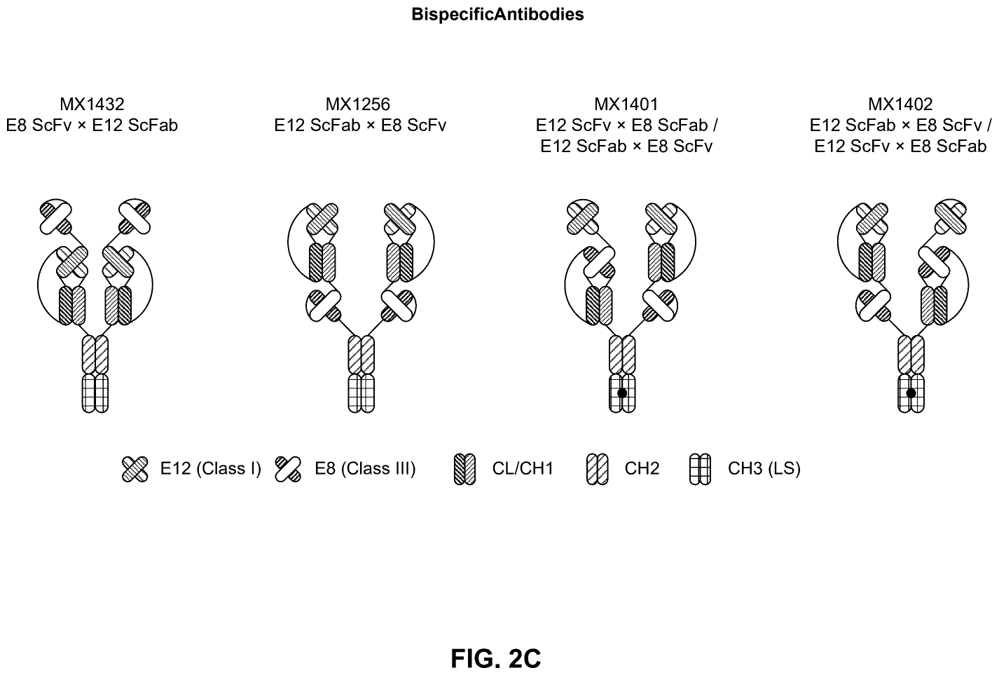

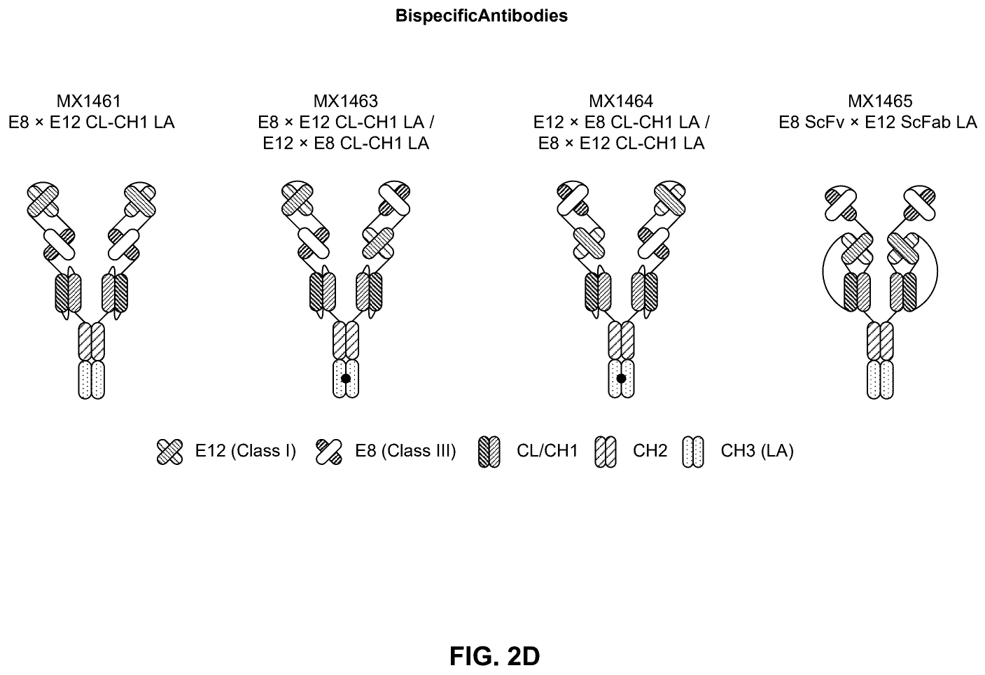

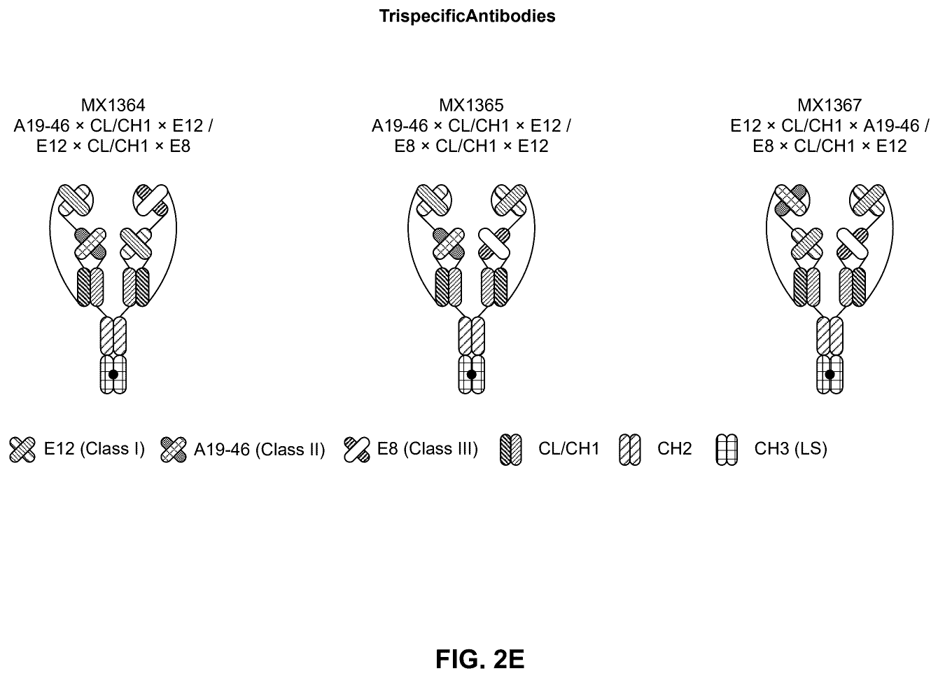

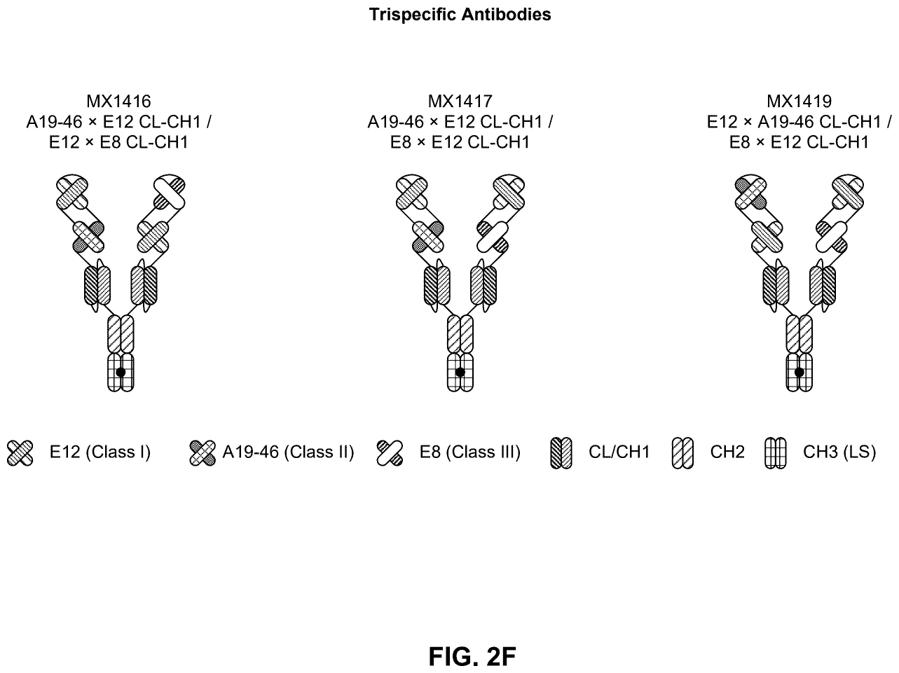

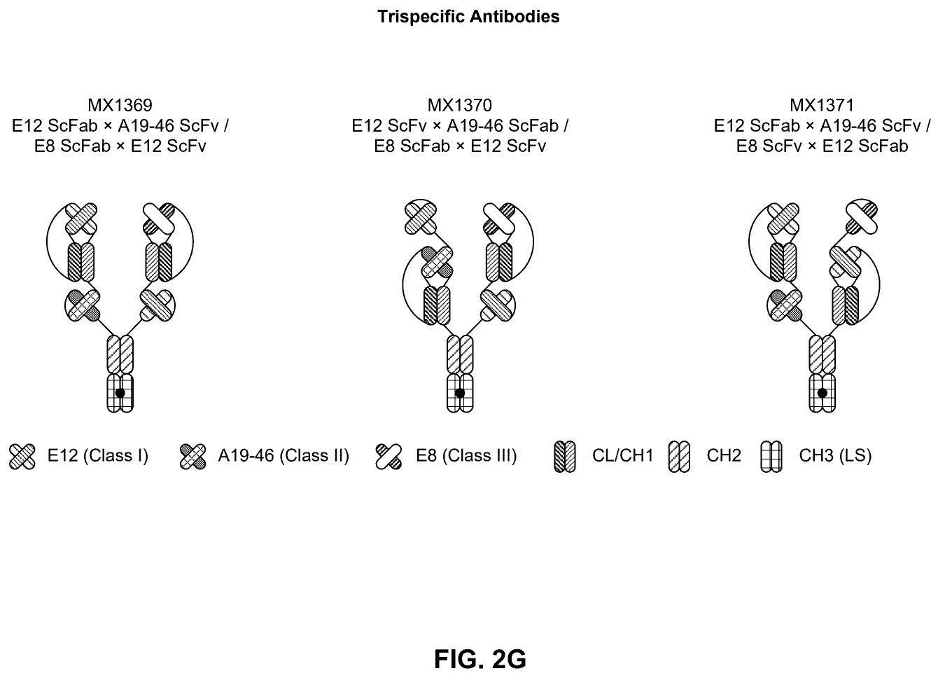

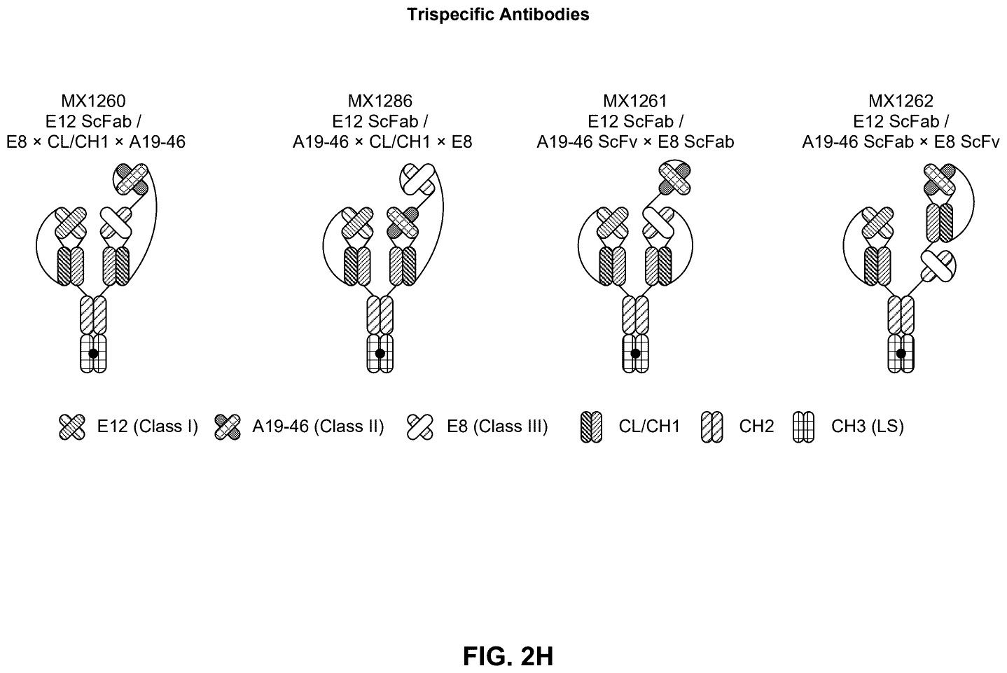

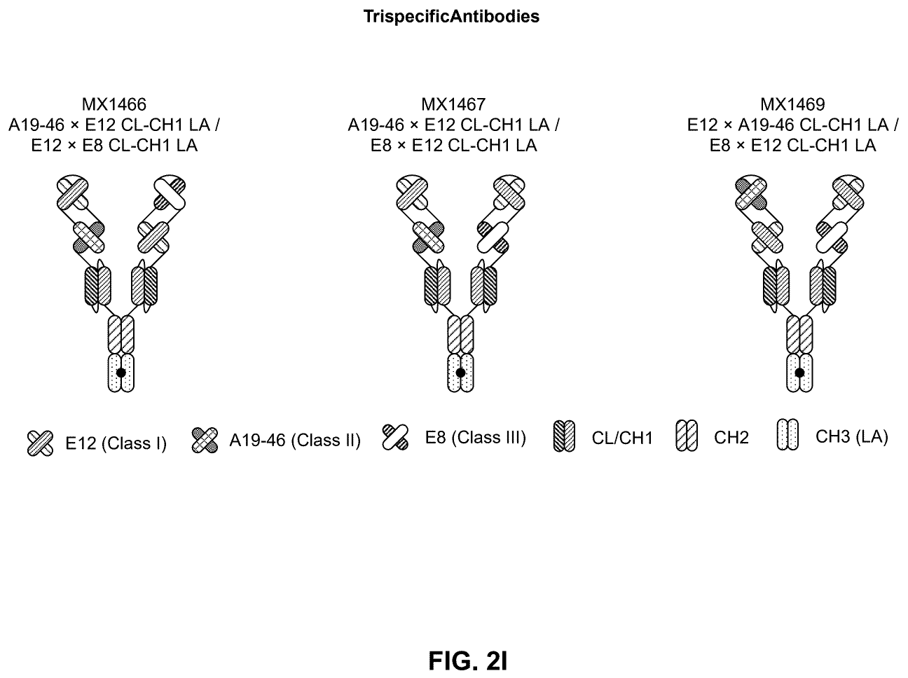

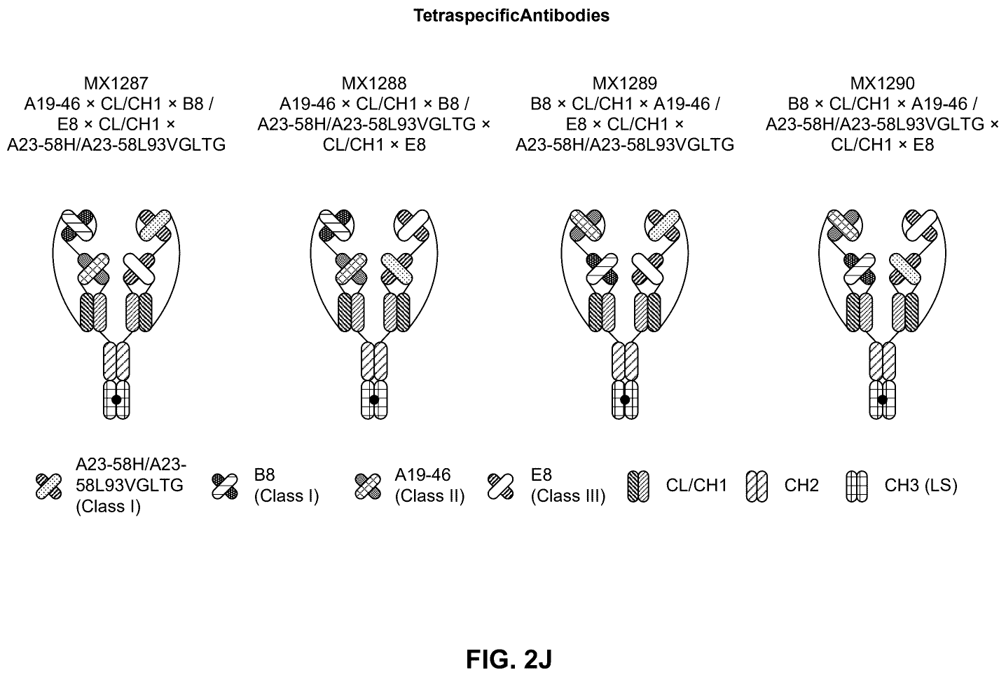

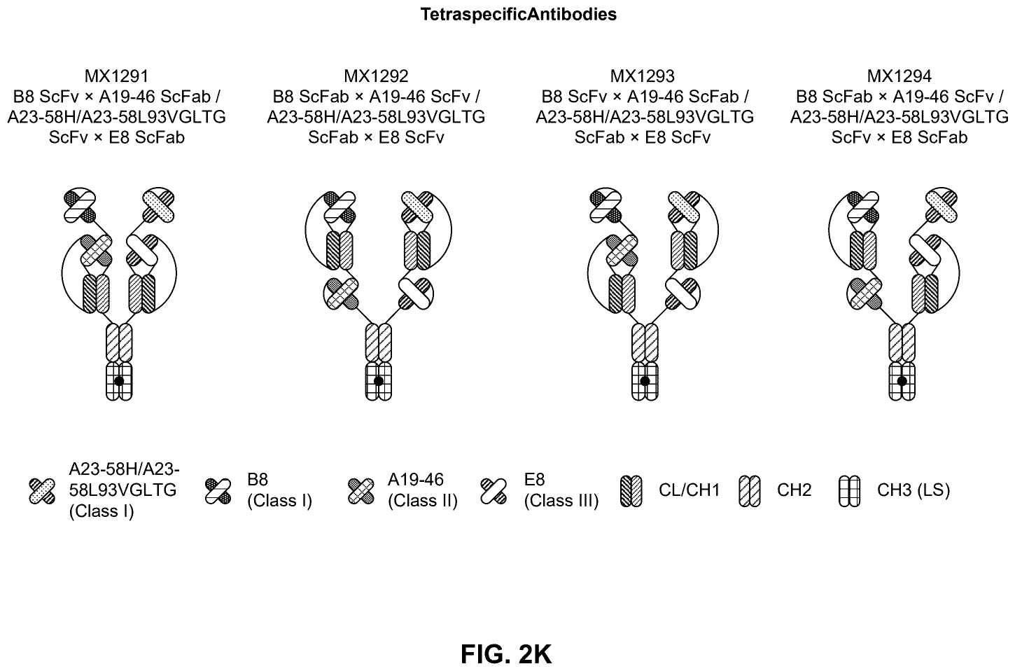

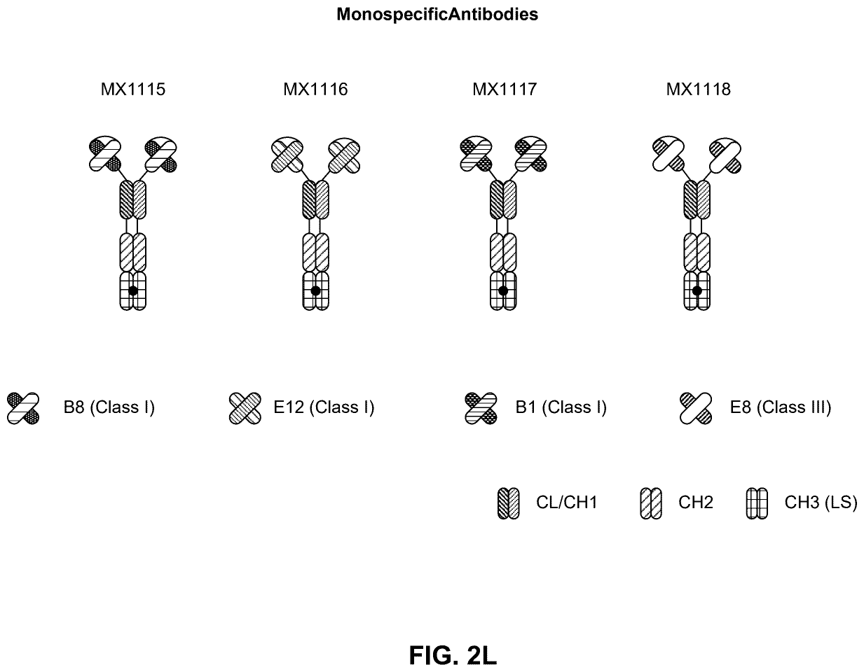

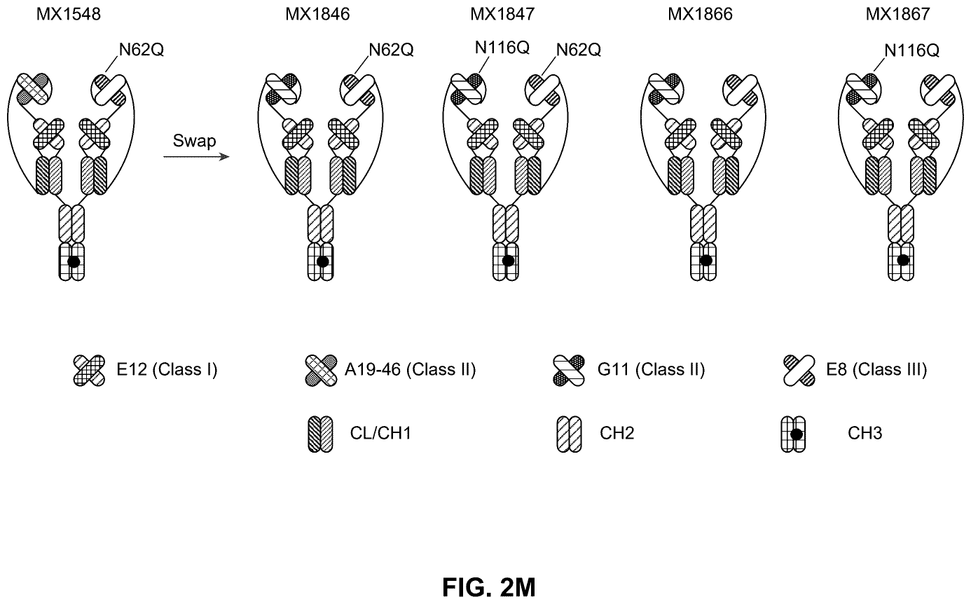

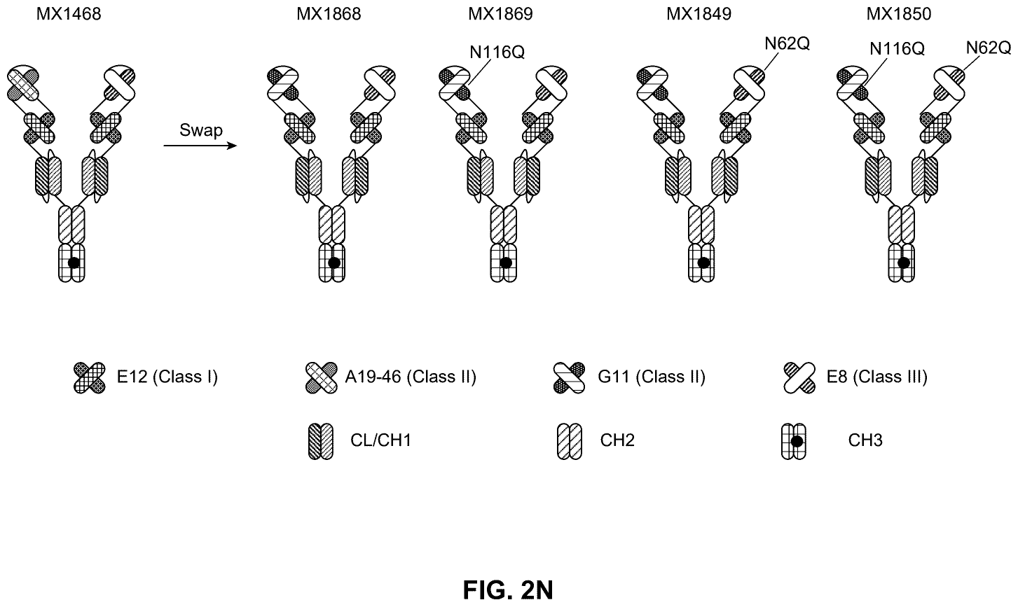

Schematics of the linear structure of the antigen binding polypeptides and of the antigen binding polypeptide complexes described herein are shown in FIGS. 1A-1UU. Schematics of the tridimensional structure of the antigen binding polypeptides and of the antigen binding polypeptide complexes described herein are shown in FIGS. 2A-2N.

Various combinations including each antibody VH and VL orientation and copy number were designed, produced, and tested for neutralizing breadth/potency and good manufacturability. In addition, the antigen binding polypeptides described herein (and therefore the antigen binding polypeptide complexes described herein) were designed to comprise an Fc (CH2-CH3) region, and one or more of the following modifications (e.g., half-life extension amino acid substitutions, knob-into-hole modifications, and N-glycosylation site modifications) were engineered into selected antigen binding polypeptides described herein (and therefore into selected antigen binding polypeptide complexes described herein):
(i) “knob” (S354C/T366W);
(ii) “hole” (Y349C/T366S/L368A/Y407V);
(iii) “LA” (L234A/L235A);
(iv) “LS” (M428L/N434S);
(v) “TM” (L234F, L235E, and P331S);
(vi) “YTE” (M252Y, S254T, and T256E); and
(vii) “N62S” or “N62Q”;
based on EU numbering scheme.

The antigen binding polypeptides described herein (and therefore the antigen binding polypeptide complexes described herein) comprise, for example, an Fc region comprising a CH2 and a CH3. In some aspects, the CH2 comprises an amino acid sequence that is at least 65%, at least 70%, at least 75%, at least 80%, at least 85%, at least 90%, at least 95%, at least 99%, or 100% identical to any one of SEQ ID NOs: 1075-1078. In some aspects, the CH3 comprises an amino acid sequence that is at least 65%, at least 70%, at least 75%, at least 80%, at least 85%, at least 90%, at least 95%, at least 99%, or 100% identical to any one of SEQ ID NOs: 1079-1085.

The antigen binding polypeptides described herein (and therefore the antigen binding polypeptide complexes described herein) comprise, for example, an Fc region comprising a sequence of SEQ ID NOs: 376-380.

Additionally, the antigen binding polypeptides described herein (and therefore the antigen binding polypeptide complexes described herein) were engineered to comprise an immunoglobulin light chain constant region (CL) and/or an immunoglobulin heavy chain constant region 1 (CH1).

The antigen binding polypeptides described herein (and therefore the antigen binding polypeptide complexes described herein) comprise, for example, a CL comprising a sequence of SEQ ID NO: 374, 637, or 1092-1095, and/or a CH1 comprising a sequence of SEQ ID NO: 375, 634, 1070, 1071, or 1086-1091. The antigen binding polypeptides described herein (and therefore the antigen binding polypeptide complexes described herein) can further comprise, one or more hinge regions of SEQ ID NO: 635, 636, 1072, 1073, or 1074, wherein a hinge region is interposed, for example, between a CH1 and a CH2 (i.e., a hinge region is localized between the carboxy terminal residue of a CH1 and the N-terminal residue of a CH2).

The different portions of the antigen binding polypeptides described herein were linked with one or more linkers.

The antigen binding polypeptides described herein (and therefore the antigen binding polypeptide complexes described herein) comprise, for example, one or more linker selected from SEQ ID NOs: 333-345 or 967-971.

### Example 3

### Synthesis, Cloning and Expression of Antigen Binding Polypeptides and Antigen Binding Polypeptide Complexes

After design of the amino acid sequences for each antigen binding polypeptide, the nucleotide sequences encoding each antigen binding polypeptide were synthesized using human preferred codons (GenScript) and cloned into eukaryotic expression vectors. For expressing each antigen binding polypeptide, the eukaryotic expression vectors were transfected into Expi293 cells (Life Technology) using Expi293 transfection reagent (Life Technology) as previously reported (DOI: 10.1126/science.aan8630). The transfected cells were cultured in a shaker incubator at 120 rpm. 37° C. 9% CO2 for 4˜5 days. For the expression of antigen binding polypeptide complexes comprising two same antigen binding polypeptides the single eukaryotic expression vector was transfected into Expi293 cells. For the expression of antigen binding polypeptide complexes comprising two different antigen binding polypeptides, equal amounts of the two eukaryotic expression vectors were transfected into Expi293 cells.

Culture supernatants were harvested and filtered, the antigen binding polypeptide complexes were purified over a Protein A (GE Health Science) column. Each antigen binding polypeptide complex was eluted with IgG elution buffer (Pierce), immediately buffer exchanged with phosphate buffered saline (PBS) and concentrated using Centricon Plus-70 (Millipore Sigma) membrane filter unit. After concentration, each antigen binding polypeptide complex was applied to a Superdex 200 16/600 size exclusion column (Cytiva) to remove aggregates and different species in the preparation. The fractions were then analyzed on reduced and non-reduced SDS-PAGE to identify the fractions that contained the antigen binding polypeptide complexes. The pooled fractions were then further concentrated, aliquoted and analyzed by SDS-PAGE as well as an analytical SEC column (Superdex 200 16/600) to verify purity by using standard procedures.

### Example 4

### Pseudovirus Neutralization Assay of Antigen Binding Polypeptide Complexes

The pseudotyped neutralization potency (IC50 and IC80) of several anti-SARS-CoV-2 antigen binding polypeptide complexes was determined, and compared to the pseudotyped neutralization potency (IC50 and IC80) of several monoclonal anti-SARS-CoV-2 antibodies or combinations thereof. Specifically. S-containing lentiviral pseudovirions were produced by co-transfection of packaging plasmid pCMVdR8.2, transducing plasmid pHR′ CMV-Luc, a TMPRSS2 plasmid and S plasmids from SARS-CoV-2 variants into 293T cells using Lipofectamine 3000 transfection reagent (L3000-001, ThermoFisher Scientific, Asheville, NC), 293T-ACE2 cells (provided by Dr. Michael Farzan) were plated into 96-well white/black Isoplates (PerkinElmer, Waltham, MA) at 7.500 cells per well the day before infection of SARS-CoV-2 pseudovirus. Serial dilutions of the anti-SARS-CoV-2 antigen binding polypeptide complexes and of the anti-SARS-CoV-2 monoclonal antibodies were mixed with titrated pseudoviruses, incubated for 45 minutes at 37° C., and added to cells in triplicate. Following 2 h of incubation, wells were replenished with 150 μl of fresh media. Cells were lysed 72 h later, and luciferase activity was measured with Microbeta (Perking Elmer). Percent neutralization and neutralization IC50 and IC80 values are shown in Tables E4 and E5. Schematics of the structures of exemplary anti-SARS-CoV-2 antigen-binding polypeptide complexes tested in this experiment are shown in FIG. 4 and FIGS. 2M-2N.

TABLE E4
pseudotyped neutralization potency (IC50 and IC80) for the tested anti-SARS-CoV-2 Antigen
Binding Polypeptide Complexes (IC50 and IC80 are expressed in nM; n.t.: not tested).

| | Omicron Sublineages | | | | | | |
|---|---|---|---|---|---|---|---|
| | Class | BA.4/5 | | BQ 1 | | BQ 1.1 | |
| MX1042 | I + III | <0.01 | 0.02 | 0.07 | 0.16 | <0.01 | 0.02 |
| MX1043 | I + II | 0.02 | 0.06 | 0.02 | 0.09 | 0.01 | 0.07 |
| MX1069 | I + II + III | <0.01 | 0.01 | <0.01 | 0.02 | 0.01 | 0.04 |
| MX1055 | I + II + III | 0.01 | 0.02 | 0.09 | 0.35 | <0.01 | 0.02 |
| MX1089 | I + II + III | 0.01 | 0.02 | 0.02 | 0.04 | 0.01 | 0.05 |
| MX1206 | I + II + III | 0.08 | 0.15 | 0.13 | 0.27 | 0.07 | 0.19 |
| MX1091 (E12 mAb) | I | 0.03 | 0.06 | 0.08 | 0.27 | 0.04 | 0.12 |
| MX1090 (B8 mAb) | I | 0.05 | 0.10 | 0.10 | 0.33 | 0.03 | 0.14 |
| MX825 | I | 0.03 | 0.09 | 0.16 | 0.58 | 0.20 | 8.78 |
| (A23-58H/L93VGLTG mAb) | | | | | | | |
| MX173 (A19-46 mAb) | II | >67 | >67 | n.t. | n.t. | 2.57 | 7.41 |
| MX1092 (E8 mAb) | III | <0.01 | <0.01 | 0.83 | 34.62 | 3.31 | 12.03 |
| LY1404 | III | 0.01 | 0.02 | n.t. | n.t. | >67 | >67 |
| A46 + E12 | I + II | 0.01 | 0.03 | 0.02 | 0.04 | 0.01 | 0.03 |
| E8 + E12 | I + III | <0.01 | <0.01 | <0.01 | 0.02 | 0.01 | 0.03 |
| A46 + E12 + B8 | I + II | n.t. | n.t. | n.t. | n.t. | n.t. | n.t. |
| A46 + E12 + B8 + E8 | I + II + III | <0.01 | 0.04 | 0.01 | 0.10 | <0.01 | 0.13 |
| A46 + E8 + B8 | I + II + III | n.t. | n.t. | 0.11 | 0.35 | 0.07 | 0.11 |
| A46 + E12 + E8 | I + II + III | n.t. | n.t. | n.t. | n.t. | n.t. | n.t. |
| A46 + E12 + B8 + A58VGLTG | I + II + III | n.t. | n.t. | n.t. | n.t. | n.t. | n.t. |
| A46 + E12 + E8 + A58VGLTG | I + II + III | n.t. | n.t. | n.t. | n.t. | n.t. | n.t |
| A46 + B8 + E8 + A58VGLTG | I + II + III | n.t. | n.t. | n.t. | n.t. | n.t. | n.t |
| | Omicron Sublineages | | | | | | |
| | Class | BA.2.75.2 | | XBB | | XBB1.5 | |
| MX1042 | I + III | <0.01 | <0.01 | 0.04 | 0.10 | 0.24 | 0.47 |
| MX1043 | I + II | 0.01 | 0.02 | 0.01 | 0.04 | 0.40 | 0.90 |
| MX1069 | I + II + III | 0.01 | 0.02 | 0.01 | 0.03 | 0.20 | 0.38 |
| MX1055 | I + II + III | 0.01 | 0.04 | 0.05 | 0.19 | 0.14 | 0.39 |
| MX1089 | I + II + III | 0.04 | 0.05 | 0.06 | 0.17 | 0.30 | 0.86 |
| MX1206 | I + II + III | 0.03 | 0.06 | 0.34 | 0.53 | 0.38 | 0.45 |
| MX1091 (E12 mAb) | I | 0.05 | 0.27 | 0.08 | 0.21 | 0.41 | 1.22 |
| MX1090 (B8 mAb) | I | 0.08 | 0.18 | 0.09 | 0.31 | 0.29 | 0.95 |
| MX825 | I | >67 | >67 | >67 | >67 | >67 | >67 |
| (A23-58H/L93VGLTG mAb) | | | | | | | |
| MX173 (A19-46 mAb) | II | >67 | >67 | n.t. | n.t. | >67 | >67 |
| MX1092 (E8 mAb) | III | 0.01 | 0.04 | 0.41 | 4.70 | 1.90 | 2.13 |
| LY1404 | III | 0.04 | 0.14 | >67 | >67 | >67 | >67 |
| A46 + E12 | I + II | 0.02 | 0.03 | 0.02 | 0.06 | 0.39 | 0.85 |
| E8 + E12 | I + III | <0.01 | <0.01 | 0.02 | 0.05 | 0.12 | 0.58 |
| A46 + E12 + B8 | I + II | n.t. | n.t. | n.t. | n.t. | 0.08 | 0.22 |
| A46 + E12 + B8 + E8 | I + II + III | <0.01 | <0.01 | 0.04 | 0.11 | n.t. | n.t. |
| A46 + E8 + B8 | I + II + III | n.t. | n.t. | 0.08 | 0.12 | n.t. | n.t. |
| A46 + E12 + E8 | I + II + III | n.t. | n.t. | n.t. | n.t. | 0.25 | 0.64 |
| A46 + E12 + B8 + A58VGLTG | I + II + III | n.t. | n.t. | n.t. | n.t. | 0.10 | 0.20 |
| A46 + E12 + E8 + A58VGLTG | I + II + III | n.t. | n.t. | n.t. | n.t. | 0.14 | 0.33 |
| A46 + B8 + E8 + A58VGLTG | I + II + III | n.t. | n.t. | n.t. | n.t. | n.t. | n.t. |

| | | Omicron Sublineages | | | |
|---|---|---|---|---|---|
| | Class | BF.7 | | CH.1.1 | |
| MX1042 | I + III | 0.11 | 0.18 | 0.27 | 0.93 |
| MX1043 | I + II | 0.33 | 0.77 | 0.26 | 1.36 |
| MX1069 | I + II + III | 0.07 | 0.17 | 0.16 | 0.61 |
| MX1055 | I + II + III | 0.09 | 0.16 | 0.74 | 5.07 |
| MX1089 | I + II + III | 0.13 | 0.37 | 1.03 | 3.60 |
| MX1206 | I + II + III | 0.12 | 0.21 | 1.28 | 3.67 |
| MX1091 (E12 mAb) | I | 0.19 | 0.59 | 0.28 | 1.04 |
| MX1090 (B8 mAb) | I | 0.13 | 0.42 | 0.27 | 0.66 |
| MX825 | I | 0.65 | 3.17 | >67 | >67 |
| (A23-58H/L93VGLTG mAb) | | | | | |
| MX173 (A19-46 mAb) | II | 35.32 | >67 | >67 | >67 |
| MX1092 (E8 mAb) | III | 0.10 | 0.21 | 33.02 | 35.92 |
| LY1404 | III | 0.02 | 0.18 | >67 | >67 |
| A46 + E12 | I + II | 0.26 | 0.45 | 0.57 | 1.38 |
| E8 + E12 | I + III | 0.08 | 0.32 | 0.74 | 1.65 |
| A46 + E12 + B8 | I + II | 0.09 | 0.23 | n.t. | n.t. |
| A46 + E12 + B8 + E8 | I + II + III | n.t. | n.t. | n.t. | n.t. |
| A46 + E8 + B8 | I + II + III | n.t. | n.t. | n.t. | n.t. |
| A46 + E12 + E8 | I + II + III | 0.08 | 0.20 | 0.47 | 0.91 |
| A46 + E12 + B8 + A58VGLTG | I + II + III | 0.12 | 0.13 | n.t. | n.t. |
| A46 + E12 + E8 + A58VGLTG | I + II + III | 0.02 | 0.10 | n.t. | n.t. |
| A46 + B8 + E8 + A58VGLTG | I + II + III | n.t. | n.t. | 0.11 | 0.52 |

TABLE E5
pseudotyped neutralization potency (IC50 and IC80) for the tested anti-SARS-CoV-2 Antigen
Binding Polypeptide Complexes (IC50 and IC80 are expressed in nM; n.t.: not tested).

| MSTAR | | | | WA.1 | | BA.2 | | BA.4/5 | |
|---|---|---|---|---|---|---|---|---|---|
| version | Class 3 | Class 2 | ID | IC50 | IC80 | IC50 | IC80 | IC50 | IC80 |
| V1 | E8 NQ | A19-46 | MX1548 | 0.091 | 0.142 | 0.020 | 0.058 | 0.050 | 0.162 |
| | E8 NQ | G11 WT | MX1846 | 0.207 | 0.685 | 0.023 | 0.081 | 0.034 | 0.102 |
| | E8 NQ | G11 NQ | MX1847 | 0.083 | 0.264 | 0.021 | 0.066 | 0.047 | 0.110 |
| | E8 WT | G11 WT | MX1866 | 0.040 | 0.227 | 0.022 | 0.106 | 0.015 | 0.065 |
| | E8 WT | G11 NQ | MX1867 | 0.079 | 0.221 | 0.038 | 0.096 | 0.015 | 0.058 |
| V2 | E8 WT | A19-46 | MX1468 | 0.031 | 0.168 | 0.031 | 0.098 | 0.059 | 0.164 |
| | E8 WT | G11 WT | MX1868 | 0.038 | 0.255 | 0.022 | 0.092 | 0.076 | 0.186 |
| | E8 WT | G11 NQ | MX1869 | 0.059 | 0.198 | 0.028 | 0.082 | 0.052 | 0.133 |
| | E8 NQ | G11 WT | MX1849 | 0.088 | 0.685 | 0.023 | 0.080 | 0.051 | 0.170 |
| | E8 NQ | G11 NQ | MX1850 | 0.112 | 0.170 | 0.020 | 0.048 | 0.030 | 0.080 |
| MSTAR | | | | BQ.1.1 | | XBB.1.5 | | EG.5 | |
| version | Class 3 | Class 2 | ID | IC50 | IC80 | IC50 | IC80 | IC50 | IC80 |
| V1 | E8 NQ | A19-46 | MX1548 | 0.011 | 0.091 | 0.10 | 0.30 | 0.33 | 1.20 |
| | E8 NQ | G11 WT | MX1846 | 0.037 | 0.093 | 0.10 | 0.22 | 0.07 | 0.24 |
| | E8 NQ | G11 NQ | MX1847 | 0.038 | 0.115 | 0.12 | 0.26 | 0.13 | 0.34 |
| | E8 WT | G11 WT | MX1866 | 0.016 | 0.070 | n.t. | n.t. | 0.34 | 0.52 |
| | E8 WT | G11 NQ | MX1867 | 0.011 | 0.049 | n.t. | n.t. | 0.19 | 0.27 |
| V2 | E8 WT | A19-46 | MX1468 | 0.025 | 0.099 | n.t. | n.t. | 0.02 | 0.04 |
| | E8 WT | G11 WT | MX1868 | 0.019 | 0.076 | n.t. | n.t. | 0.16 | 0.18 |
| | E8 WT | G11 NQ | MX1869 | 0.025 | 0.106 | n.t. | n.t. | 0.10 | 0.20 |
| | E8 NQ | G11 WT | MX1849 | 0.039 | 0.131 | 0.11 | 0.21 | 0.04 | 0.18 |
| | E8 NQ | G11 NQ | MX1850 | 0.021 | 0.116 | 0.09 | 0.14 | 0.03 | 0.14 |

| MSTAR | | | | XBB.2.3.2 (VRC) | | BA.2.86 (VRC) | |
|---|---|---|---|---|---|---|---|
| version | Class 3 | Class 2 | ID | IC50 | IC80 | IC50 | IC80 |
| V1 | E8 NQ | A19-46 | MX1548 | 0.051 | 0.200 | 0.100 | 0.370 |
| | E8 NQ | G11 WT | MX1846 | 0.016 | 0.072 | 0.086 | 0.320 |
| | E8 NQ | G11 NQ | MX1847 | 0.021 | 0.070 | 0.130 | 0.390 |
| | E8 WT | G11 WT | MX1866 | n.t. | n.t. | n.t. | n.t. |
| | E8 WT | G11 NQ | MX1867 | n.t. | n.t. | n.t. | n.t. |
| V2 | E8 WT | A19-46 | MX1468 | 0.014 | 0.045 | 0.037 | 0.140 |
| | E8 WT | G11 WT | MX1868 | n.t. | n.t. | n.t. | n.t. |
| | E8 WT | G11 NQ | MX1869 | n.t. | n.t. | n.t. | n.t. |
| | E8 NQ | G11 WT | MX1849 | 0.160 | 0.460 | 0.690 | 1.800 |
| | E8 NQ | G11 NQ | MX1850 | 0.037 | 0.160 | 0.410 | 1.300 |

### Example 5

### Pharmacokinetics (PK) of Antigen Binding Polypeptide Complexes in Humanized FcRn Mice

Human FcRn transgenic mice (C57BL/6, B6.mFcRn^{-/-} hFcRn Tg32 line from The Jackson Laboratory) were used to assess the pharmacokinetics of the antigen binding polypeptide complexes showing good pseudotyped neutralization potency (IC50 and IC80). Each animal was infused intravenously with 5 mg antibody/kg of body weight. Whole blood samples were collected at day 1, 2, 5, 7, 9, 14, 21, 28, 35, 42, 49, and 56. Serum was separated by centrifugation. Serum antibody levels were measured by ELISA. All mice were bred and maintained under pathogen-free conditions at an American Association for Assessment and Accreditation of Laboratory Animal Care International-accredited animal facility at the National Institute of Allergy and Infectious Diseases and housed in accordance with the procedures outlined in the Guide for the Care and Use of Laboratory Animals. All mice were between 6 and 13 weeks of age.

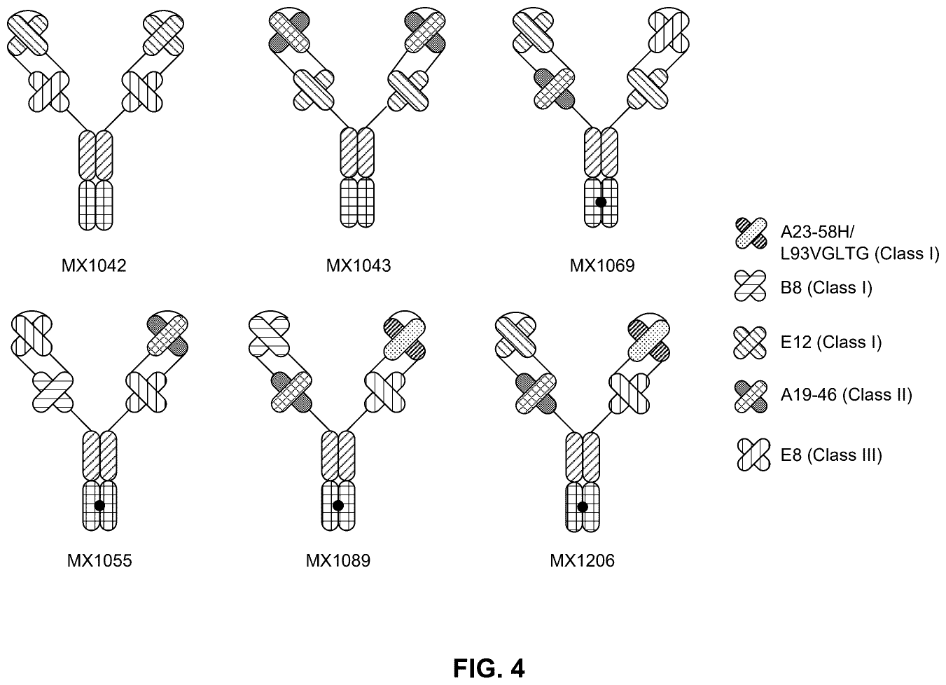

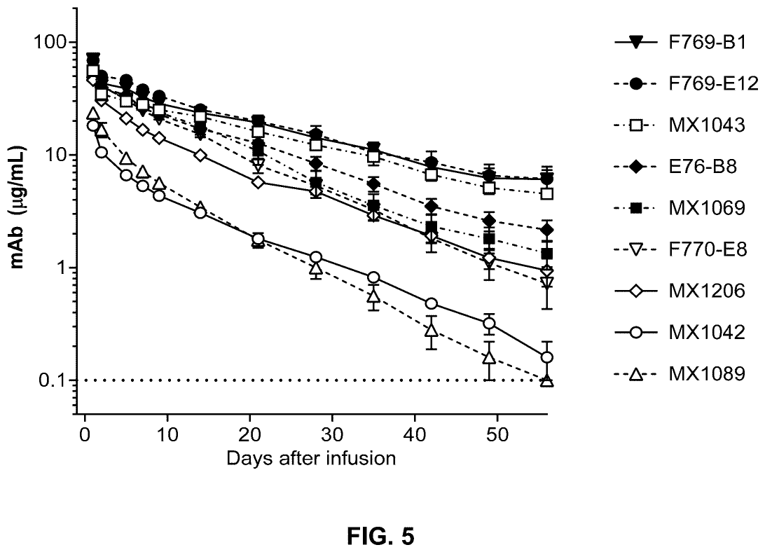

The PK was determined for each of the tested antigen binding polypeptide complexes and monoclonal antibodies, and plotted as shown in FIG. 5. The assay showed that MX1042 and MX1089 did not reach the same peak level as the other tested antigen binding polypeptide complexes and had faster clearance.

### Example 6

Passive Transfer into Syrian Hamsters Assay Using Antigen Binding Polypeptide Complexes in Humanized FcRn Mice

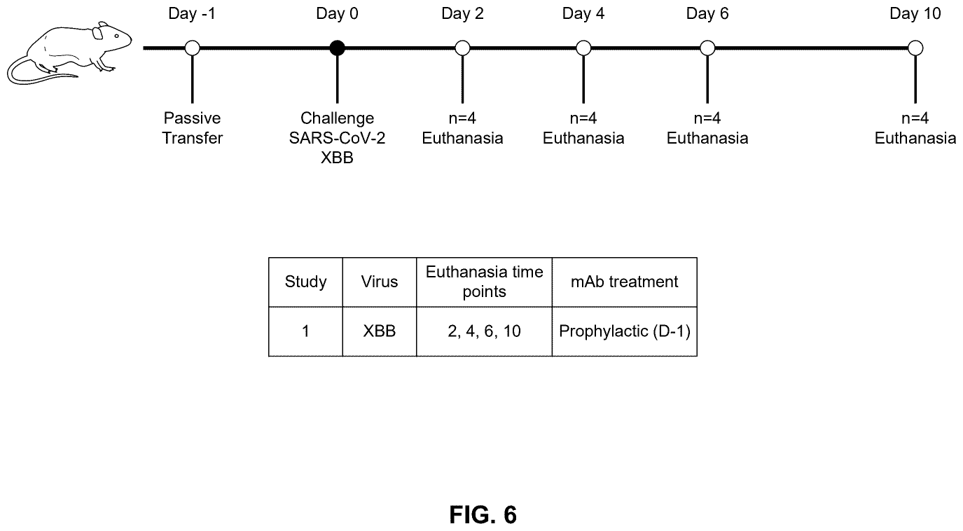

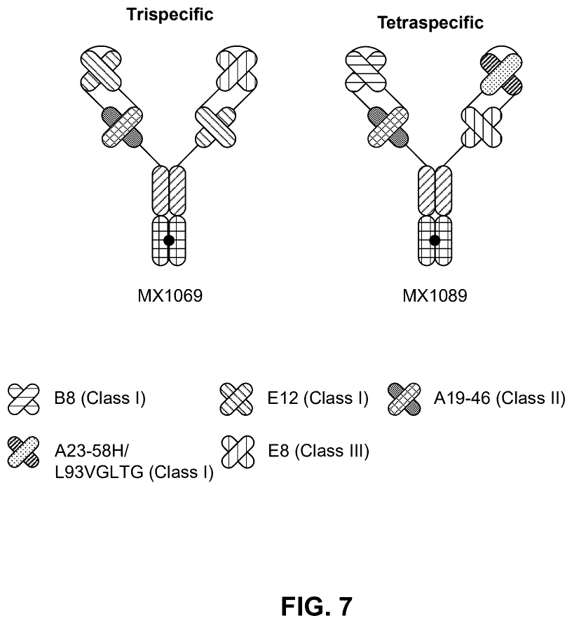

Well performing antigen binding polypeptide complexes as determined by the experiments described in the preceding examples, were tested in Syrian Hamsters as schematized in FIG. 6. Briefly, passive transferred (day-1) Syrian Hamsters were challenged with SARS-CoV-2 virus and euthanized at different time points (2, 4, 6, and 10 days). The animals were treated with 10 mg/kg of MX1069 (trispecific) or with 10 mg/kg of MX1089 (tetraspecific). Control animals were treated with PBS or with 10 mg/kg of E12 and E8 anti-SARS-CoV-2 monoclonal antibodies. Schematics of the structures of MX1069 and MX1089 is shown in FIG. 7.

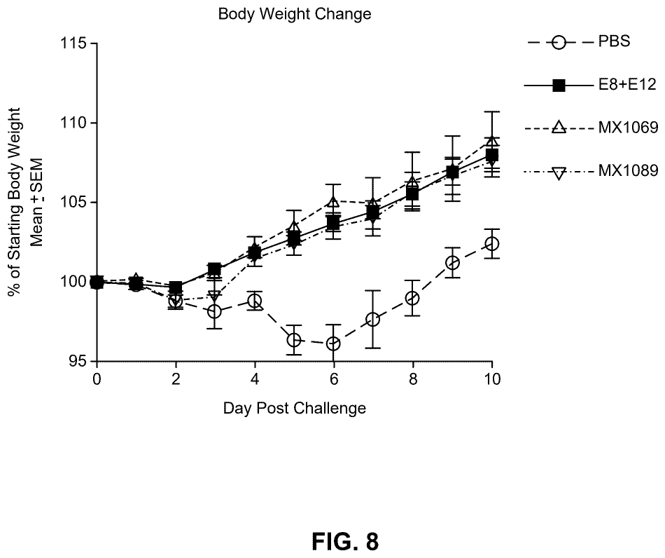

The body weight change was measured for treated and control animals, and showed that both MX1069 and MX1089 protected against weight loss (FIG. 8).

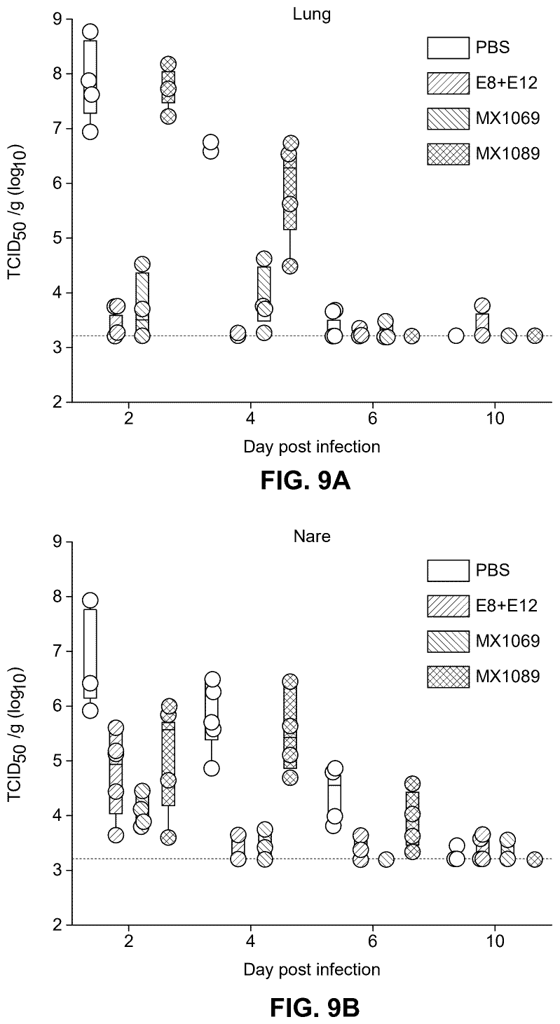

The Median Tissue Culture Infectious Dose (TCID50) was also tested in lungs and nares, MX1069 showed reduced lung viral titers (FIG. 9A-9B).

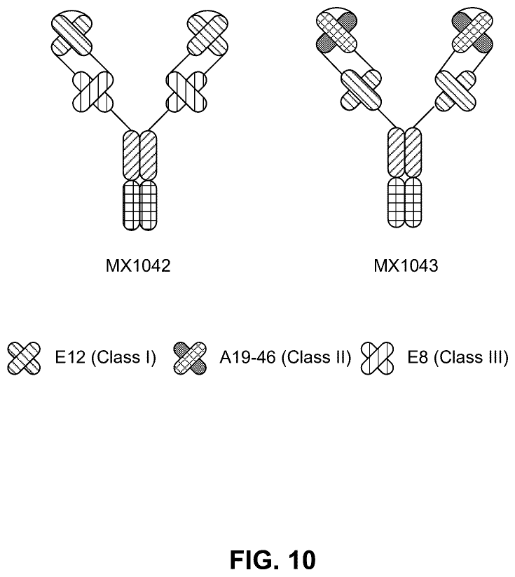

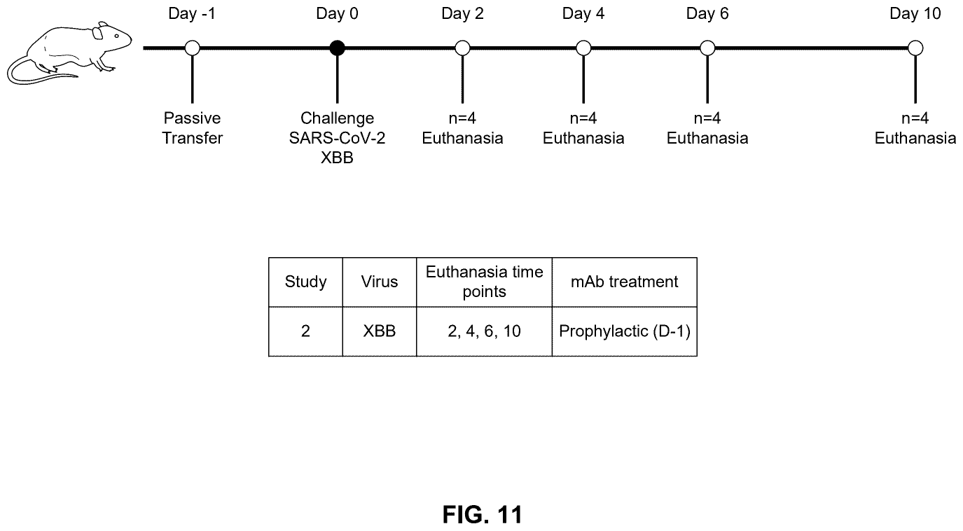

MX1042 and MX1043 (bispecific, schematics of the structures shown in FIG. 10) were also analogously tested in Syrian Hamsters. The experimental design is schematized in FIG. 11. The animals were treated with 10 mg/kg of MX1042, with 10 mg/kg of MX1043, or with 10 mg/kg of MX1042+MX1043. Control animal were treated with PBS.

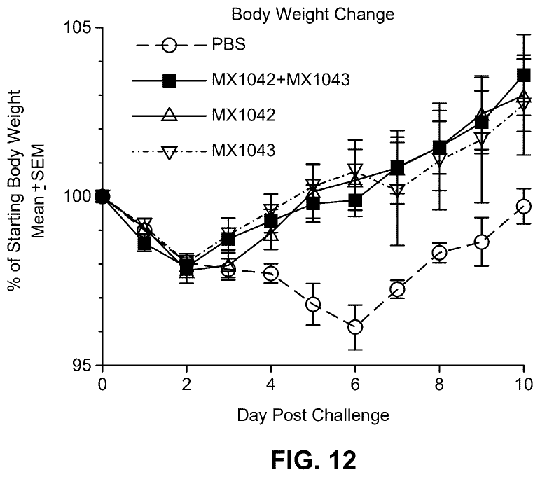

The body weight change was measured for treated and control animals, and showed that both MX1042 and MX1043 protected against weight loss (FIG. 12).

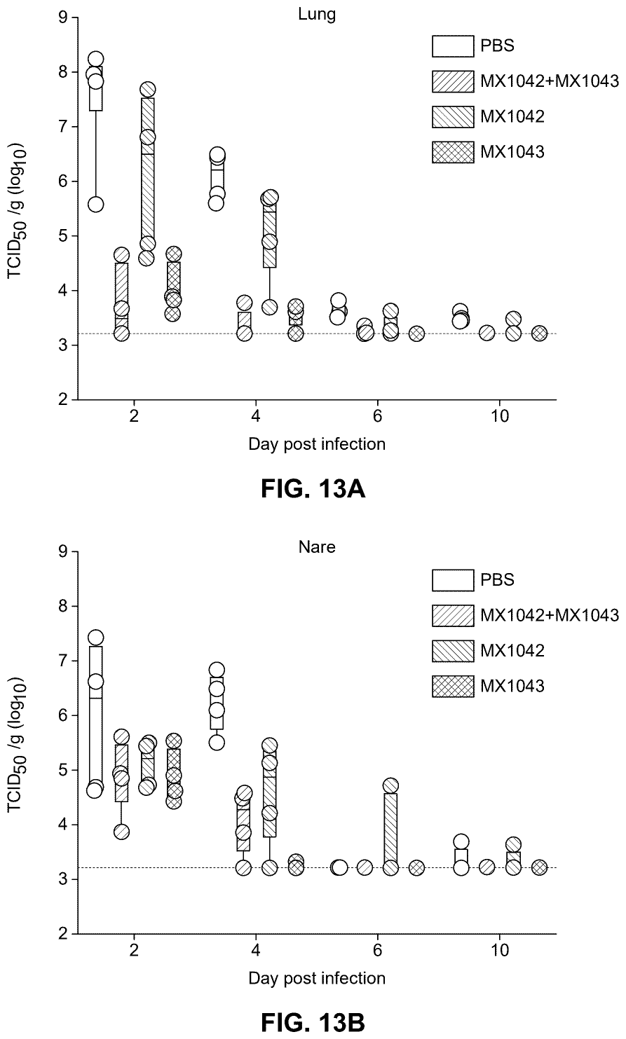

The Median Tissue Culture Infectious Dose (TCID50) was also tested in lungs and nares, MX1043 and the MX1042+MX1043 combination both reduced lung viral titers (FIG. 13A-13B).

### Example 7

### Determination of the Isoelectric Point (pI) of the Antigen Binding Polypeptide Complexes

The isoelectric point of MX1042, MX1043, MX1069, MX1089, MX1206, and MX1091 (E12 mAb) was determined. The samples were first diluted with water to a concentration of 1 mg/mL. The diluted samples were then mixed with a solution containing 1% methyl cellulose, two pharmalytes (pH 3-10 and pH 6-8), and two pI markers (5.85 and 9.99). The cIEF analysis was performed using a Maurice System equipped with a Maurice cIEF separation cartridge (ProteinSimple). The proteins were focused and separated by pre-focusing for 1 min at 1500 V, followed by focusing at 3000 V for 8 min. Native protein fluorescent signals were used for peak integration and apparent pI determination. Table E6 summarizes the pI, neutralization profile, binding profile, and additional chemical-physical properties of the well performing antigen binding polypeptide complexes.

TABLE E6
pI, neutralization profile, binding profile, and additional chemical-physical properties of the well
performing antigen-binding polypeptide complexes (Risk Level : Low ✓, Medium # , High X).

| | Candidate #1 | Candidate #2 | Candidate #3 | Candidate #4 | Candidate #5 | Candidate #6 |
|---|---|---|---|---|---|---|
| ID# | MX1042 | MX1043 | MX1069 | MX1089 | MX1206 | MX1091 |
| | (bispecific) | (bispecific) | (trispecific) | (tetraspecific) | (tetraspecific) | (E12 mAb) |
| Neutralization profile | BQ1, BQ.1.1 | BQ1, BQ.1.1 | BQ1, BQ.1.1 | BQ1, BQ.1.1 | BQ1, BQ.1.1 | BQ1, BQ.1.1 |
| | XBB, XBB.1.5, | XBB, XBB.1.5, | XBB, XBB.1.5, | XBB, XBB.1.5, | XBB, XBB.1.5, | XBB, XBB.1.5, |
| | CH.1.1 ✓ | CH.1.1 ✓ | CH.1.1 ✓ | CH.1.1 # | CH.1.1 # | CH.1.1 ✓ |
| Binding profile to | positive \|BLI\| | positive BLI | positive BLI | positive BLI | positive BLI | positive BLI |
| target antigens | [PM1 ]binding | binding to | binding to | binding to | binding to | binding to |
| | to COVID RBD | COVID RBD | COVID RBD | COVID RBD | COVID RBD | COVID RBD |
| In silico liability | ✓ | ✓ | ✓ | ✓ | ✓ | ✓ |
| analysis | | | | | | |
| STAR Expression, | ✓ | ✓ | ✓ | ✓ | ✓ | ✓ |
| yield, SEC | | | | | | |
| \|HIC\|_{[PM2]} | 2 main peaks | Low peak and | homogenous | homogenous | homogenous | homogenous |
| | | high retention | and relative | and relative | and relative | and relative |
| | | | short retention | short retention | short retention | short retention |
| | | | time | time | time | time |
| cIEF/PI | 6.92 | 6.99 | 6.73 | 8.36 | 7.8 | 7.92 |
| Solubility | | >100 mg/ml, | ✓ | >100 mg/ml | >100 mg/ml | >100 mg/ml |
| | | self-interaction | | | | |
| Freeze/thaw stability | Requires PS80 | Requires PS80 | ✓ | Requires PS80 | Requires PS80 | Requires PS80 |
| (5X) | ✓ | # | | ✓ | ✓ | ✓ |
| Thermostability | Tm <60° C. | <60° C. | <60° C. | <60° C. | <60° C. | ✓ |
| pH stability (pH3.5 | ✓ | ✓ | ✓ | ✓ | ✓ | ✓ |
| and pH 8) | | | | | | |
| pH 6, 40° C. stress | 7.6% HMW 3w | ✓ | 14% HMW 3w | ~23.2% HMW | 9.6% HMW 3w | ✓ |
| | | | | 3w | | |
| Serum stability | Stable in | Stable in | Stable in | Stable in | Stable in | Stable in |
| | serum-no | serum-no | serum-no | serum-no | serum-no | serum-no |
| | change to | change to | change to | change to | change to | change to |
| | aSEC or BLI | aSEC or BLI | aSEC or BLI | aSEC or BLI | aSEC or BLI | aSEC or BLI |
| | binding | binding | binding | binding | binding | binding |
| Platelet binding | No platelet | No platelet | No platelet | No platelet | No platelet | No platelet |
| | binding | binding | binding | binding | binding | binding |
| rVSV Viral escape | | | | | | |
| PK in humanized | X | ✓ | ✓ | X | # | ✓ |
| FcRn mice (VRC) | | | | | | |
| Hamster challenge | X | ✓ | ✓ | X | | |
| (VRC) | | | | | | |
| Just Evotec- | ✓ | ✓ | ✓ | ✓ | ✓ | ✓ |
| polyreactivity | | | | | | |
| Just Evotec-thermal | # | # | # | # | # | ✓ |
| hold | | | | | | |
| Just Evotec-chemical | # | # | # | # | # | ✓ |
| unfolding | | | | | | |
| Just Evotec-SINS | ✓ | X | ✓ | ✓ | ✓ | ✓ |
| Just Evotec-colloidal | ✓ | X | ✓ | ✓ | ✓ | ✓ |
| instability | | | | | | |
| Just Evotec-solubility | ✓ | # | ✓ | ✓ | ✓ | ✓ |

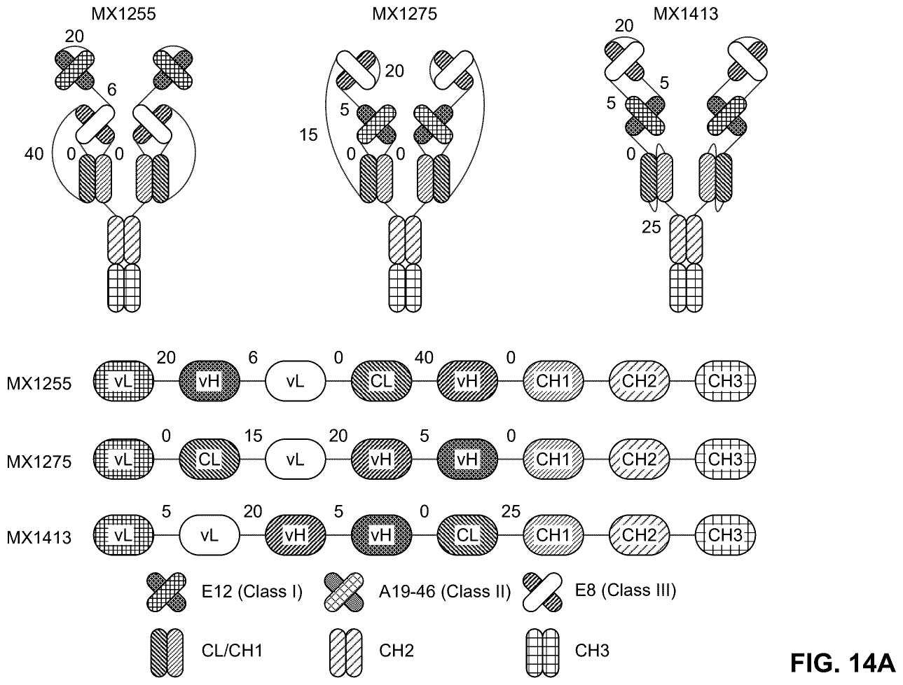

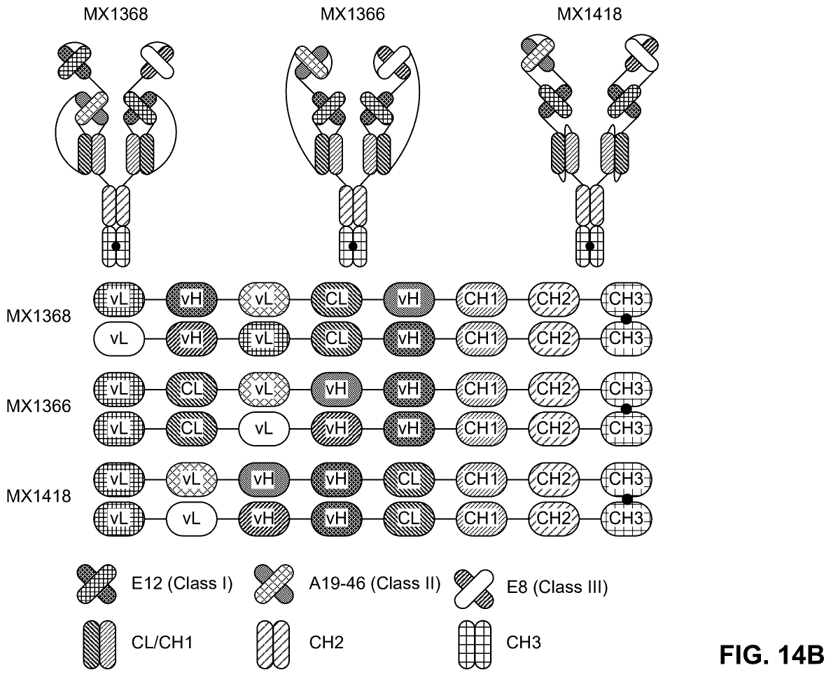

Additional candidates (MX1255, MX1275, MX1413, MX1368, MX1366, and MX1418) were similarly tested. The structures of the additional candidates are shown in FIGS. 14A-14B. Further, variants of MX1255, MX1275, MX1413, MX1368, MX1366, and MX1418 comprising a LA modification were also tested.

Tables E7 and E8 summarize the pI, neutralization profile, binding profile, and additional chemical-physical properties of the additional candidate antigen binding polypeptide complexes and of their LA variants.

TABLE E7
pI, neutralization profile, binding profile, and additional chemical-physical properties of the
additional candidate antigen-binding polypeptide complexes (Risk Level : Low ✓ , Medium #, High X).

| | Candidate #1 | Candidate #2 | Candidate #3 | Candidate #4 | Candidate #5 | Candidate #6 | Candidate #7 |
|---|---|---|---|---|---|---|---|
| ID# | MX1255 | MX1275 | MX1413 | MX1368 | MX1366 | MX1418 | MX1091 (E12 |
| | | | | | | | mAb) |
| Format | ScFv- | MSTAR- | MSTAR- | ScFv- | MSTAR- | MSTAR- | Regular Ig |
| | ScFab_LS | CL/CH1_v1_LS | CL/CH1_v2 | ScFab_LS | CL/CH1_v1_LS | CL/CH1_v2 | |
| Specificity | Bispecific | Bispecific | Bispecific | Trispecific | Trispecific | Trispecific | Monoclonal |
| Titer | 60 mg/L | 55 mg/L | 22.46 mg/L | 63 mg/L | 66 mg/L | 36.07 mg/L | ✓ |
| Neutralization | More potent | Comparable | More potent | Comparable | Comparable | More potent | ✓ |
| profile | than MX1042 | to MX1042 | than MX1042 | to MX1069 | to MX1069 | than MX1069 | |
| Binding to RBD | positive BLI | positive BLI | | | | | positive BLI |
| | binding to | binding to | | | | | binding to |
| | COVID RBD | COVID RBD | | | | | COVID RBD |
| aSEC | 99.5% | 98.3% | 99.02% | 98.7% | 99.5% | 97.65% | ✓ |
| HIC | 2 peaks | 2 peaks | | | | | |
| | homogenous | homogenous | | | | | |
| | and relative | and relative | | | | | |
| | short retention | short retention | | | | | |
| | time | time | | | | | |
| clEF/pl | 8.54 | 8.54 | | 8.49 | 8.48 | | 7.93 |
| Solubility (>100 | | 65 mg/ml | | | | | ✓ |
| mg/ml) | | | | | | | |
| Freeze/ | | | | | | | ✓ |
| thaw stability | | | | | | | |
| (5X) | | | | | | | |
| Thermostability | | | | | | | ✓ |
| pH stability | | | | | | | ✓ |
| (pH3.5 and pH | | | | | | | |
| 8) | | | | | | | |
| pH 6, 40° C. | | | | | | | ✓ |
| stress | | | | | | | |
| Serum stability | | | | | | | Stable in |
| | | | | | | | serum-no |
| | | | | | | | change to |
| | | | | | | | aSEC or BLI |
| | | | | | | | binding |
| Platelet binding | | | | | | | No platelet |
| | | | | | | | binding |
| rVSV Viral | | | | | | | |
| escape | | | | | | | |
| PK in | | | | | | | ✓ |
| humanized | | | | | | | |
| FcRn mice | | | | | | | |
| Hamster | | | | | | | |
| challenge (VRC) | | | | | | | |
| LS vs LA | LA available | LA available | LA available | LA available | LA available | LA available | |
| | as MX1452 | as MX1448/ | as MX1462 | as MX1454 | as MX1450/ | as MX1468 | |
| | | MX1451 | | | MX1453 | | |

TABLE E8
pI, neutralization profile, binding profile, and additional chemical-physical properties of the
additional candidate antigen binding polypeptide complexes (Risk Level: Low ✓ , Medium #, High X)

| | Candidate #1 | Candidate #2 | Candidate #3 | Candidate #4 | Candidate #5 | Candidate #6 | Candidate #7 |
|---|---|---|---|---|---|---|---|
| ID# | MX1452 | MX1448/ | MX1462 | MX1454 | MX1450/ | MX1468 | MX1091 (E12 |
| | | MX1451 | | | MX1453 | | mAb) |
| Format | ScFv- | MSTAR- | MSTAR- | ScFv- | MSTAR- | MSTAR- | Regular Ig |
| | ScFab_LA | CL/CH1_v1_LA | CL/CH1_v2_LA | ScFab_LA | CL/CH1_v1_LA | CL/CH1_v2_LA | |
| Specificity | Bispecific | Bispecific | Bispecific | Trispecific | Trispecific | Trispecific | Monoclonal |
| Titer | | 60 mg/L | | ✓ | 85 mg/L | | ✓ |
| Neutralization | | | | | | | ✓ |
| profile | | | | | | | |
| Binding to RBD | | | | | | | positive BLI |
| | | | | | | | binding to |
| | | | | | | | COVID RBD |
| aSEC | 99.85% | 99.9%/ | | 98.69% | 99.56% | | ✓ |
| | | 99.6% | | | | | |
| HIC | | | | | | | homogenous |
| | | | | | | | and relative |
| | | | | | | | short retention |
| | | | | | | | time |
| clEF/pl | | | | | | | 7.93 |
| Solubility (>100 | | | | | | | ✓ |
| mg/ml) | | | | | | | |
| Freeze/thaw | | | | | | | ✓ |
| stability (5X) | | | | | | | |
| Thermostability | | | | | | | ✓ |
| pH stability | | | | | | | ✓ |
| (pH3.5 and pH | | | | | | | |
| 8) | | | | | | | |
| pH 6, 40° C. | | | | | | | |
| stress | | | | | | | |
| Serum stability | | | | | | | Stable in |
| | | | | | | | serum-no |
| | | | | | | | change to |
| | | | | | | | aSEC or BLI |
| | | | | | | | binding |
| Platelet binding | | | | | | | No platelet |
| | | | | | | | binding |
| rVSV Viral | | | | | | | |
| escape | | | | | | | |
| PK in | | | | | | | ✓ |
| humanized | | | | | | | |
| FcRn mice | | | | | | | |

Hamster
challenge (VRC)

### Example 8

### Determination of the Isoelectric Point (pI) of the Antigen Binding Polypeptide Complexes

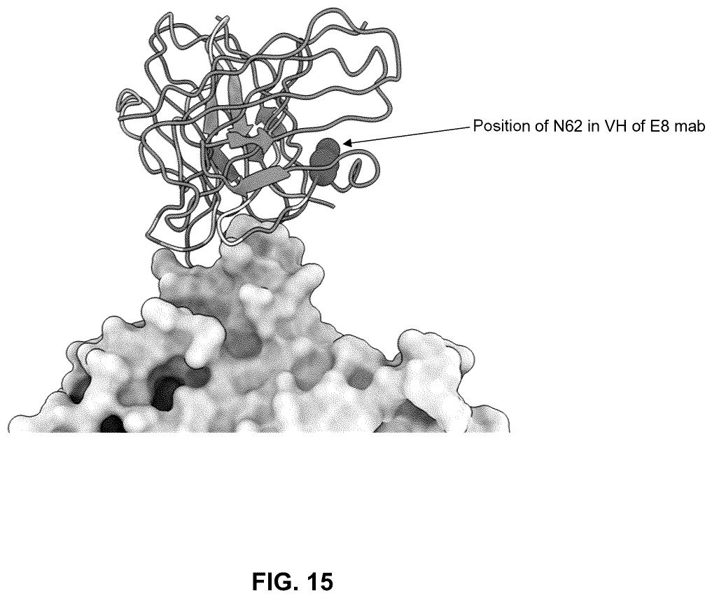

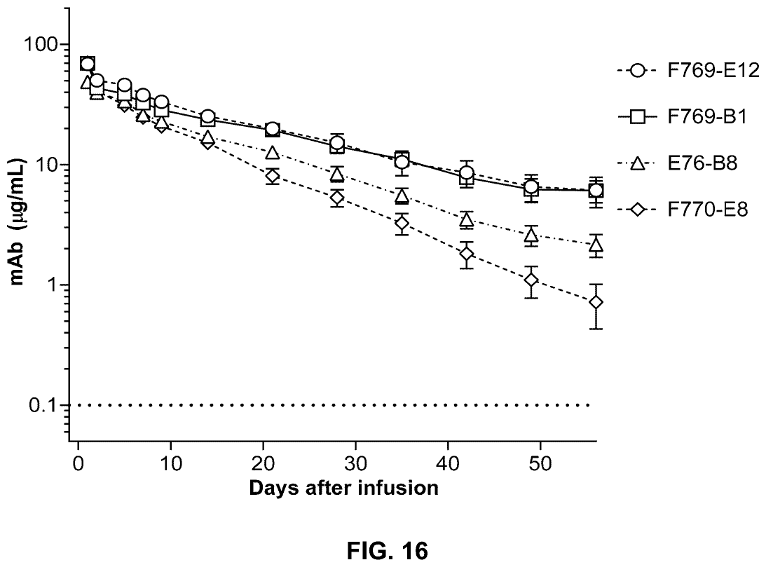

Residue 62 was shown to be glycosylated with mostly high mannose and to display the worst PK among all parental monoclonal antibodies. The position of the N62 residue in the VH of the E8 monoclonal antibody is shown in FIG. 15, FIG. 16 shows the PK of the indicated monoclonal antibodies. To improve their PK, the N62Q glycosylation mutation was incorporated into the antigen binding polypeptides described herein (and therefore into the antigen binding polypeptide complexes described herein). For example, the N62Q glycosylation mutation was incorporated into MX1448 to obtain MX1545, and into MX1450 to obtain MX1548. The antigen binding complexes comprising the N62Q glycosylation mutation were further tested as shown in Example 9.

### Example 9

### PK of Additional Candidate Antigen Binding Polypeptide Complexes in Humanized FcRn Mice

Human FcRn transgenic mice (C57BL/6, B6.mFcRn^{-/-} hFcRn Tg32 line from The Jackson Laboratory) were used to assess the pharmacokinetics of the antigen binding polypeptide complexes showing a good pseudotyped neutralization potency (IC50 and IC80). Each animal was infused intravenously with 5 mg antibody/kg of body weight. Whole blood samples were collected at day 1, 2, 5, 7, 9, 14, 21, 28, 35, 42, 49, and 56. Serum was separated by centrifugation. Serum antibody levels were measured by ELISA. All mice were bred and maintained under pathogen-free conditions at an American Association for Assessment and Accreditation of Laboratory Animal Care International-accredited animal facility at the National Institute of Allergy and Infectious Diseases and housed in accordance with the procedures outlined in the Guide for the Care and Use of Laboratory Animals. All mice were between 6 and 13 weeks of age.

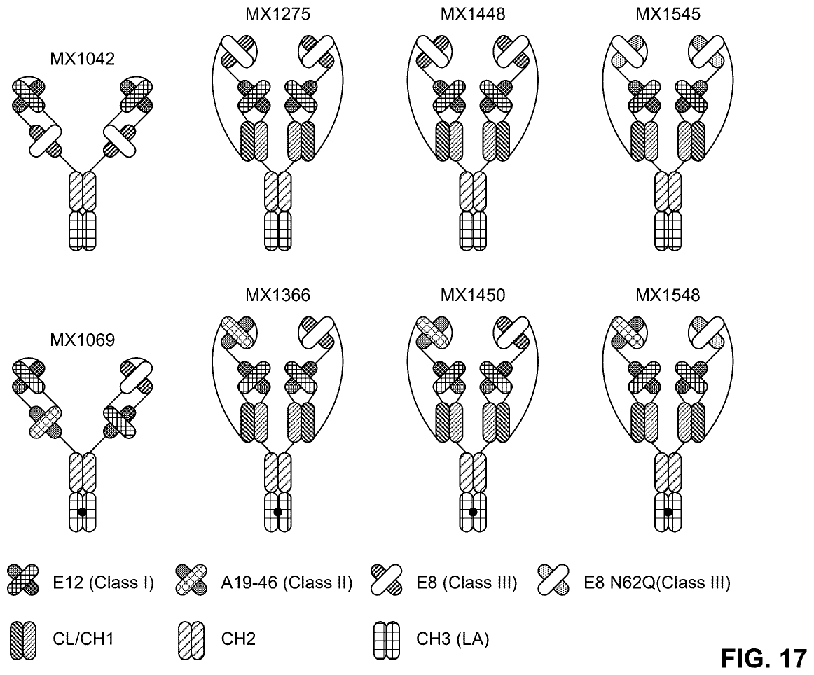

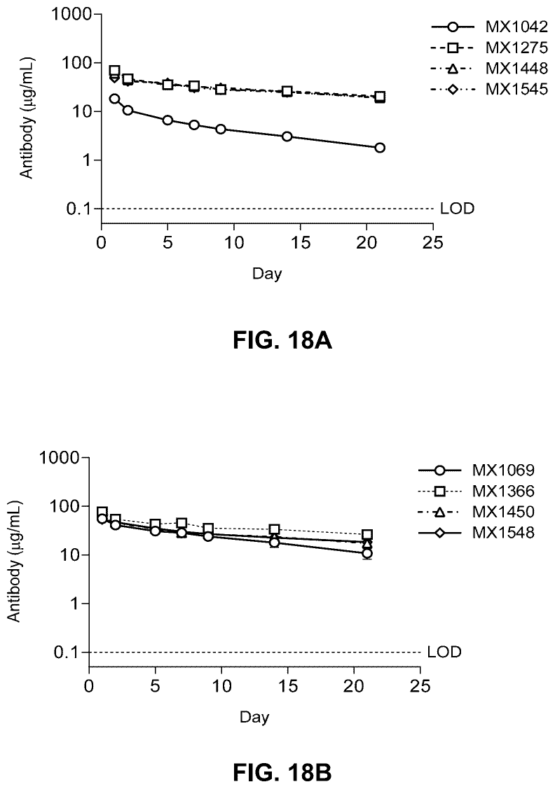

The PK was determined for each candidate antigen binding polypeptide complex, including antigen binding polypeptide complexes comprising the N62Q glycosylation mutation, and antigen binding polypeptide complexes comprising the LA and LS Fc modifications. Briefly: MX1042; MX1275 corresponding to MX1042, but further comprising a CL/CH1 region and a LS modification; MX1448 corresponding to MX1275, but further comprising a LA modification; MX1545 corresponding to MX1448, but further comprising a N62Q glycosylation mutation; MX1069; MX1366 corresponding to MX1069, but further comprising a CL/CH1 region and a LS modification; MX1450 corresponding to MX1366, but further comprising a LA modification; and MX1548 corresponding to MX1450, but further comprising a N62Q glycosylation mutation were tested. Schematics of the structures of the tested candidates (and controls: MX1042 and MX1069) are shown in FIG. 17. The results shown that the antigen binding polypeptide complexes comprising the CL/CH1 regions had improved PK compared to the control not comprising the CL/CH1 regions (FIG. 18A-18B).

### Example 10

In vitro Production of mRNAs Encoding the Antigen Binding Polypeptide Complexes

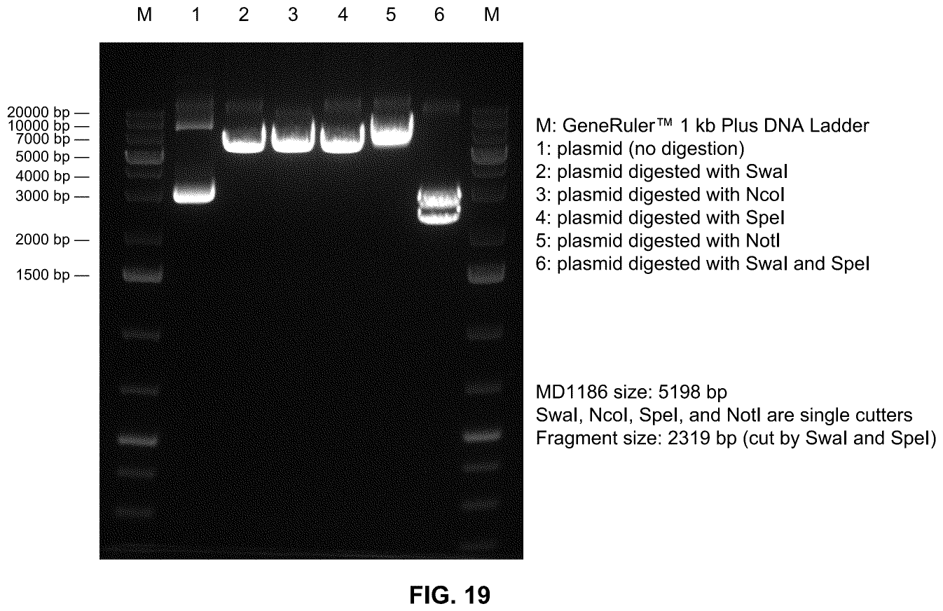

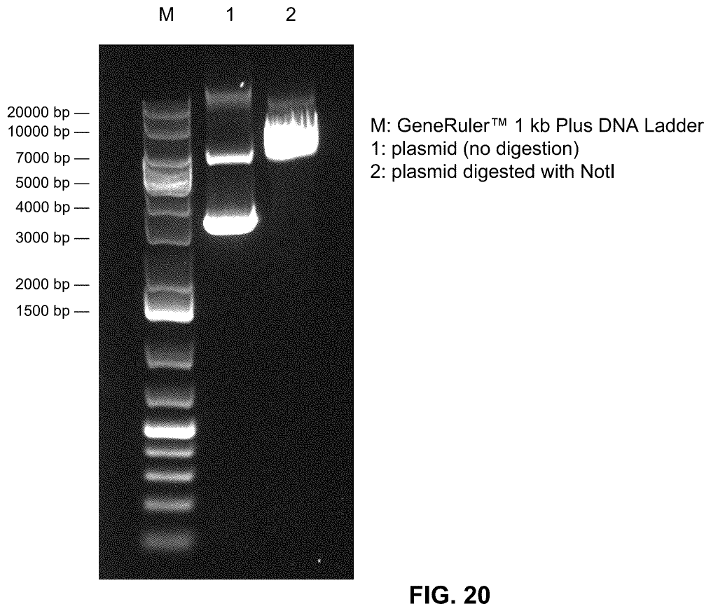

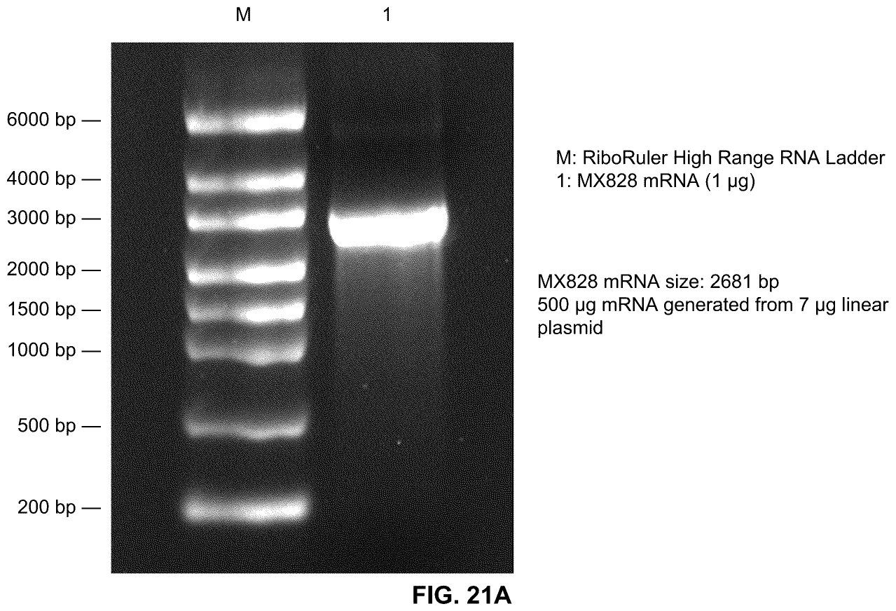

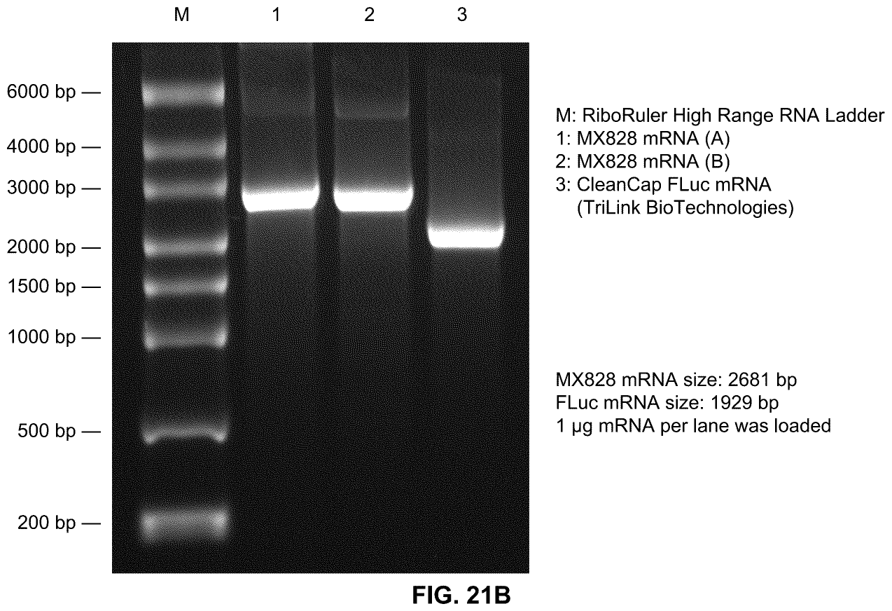

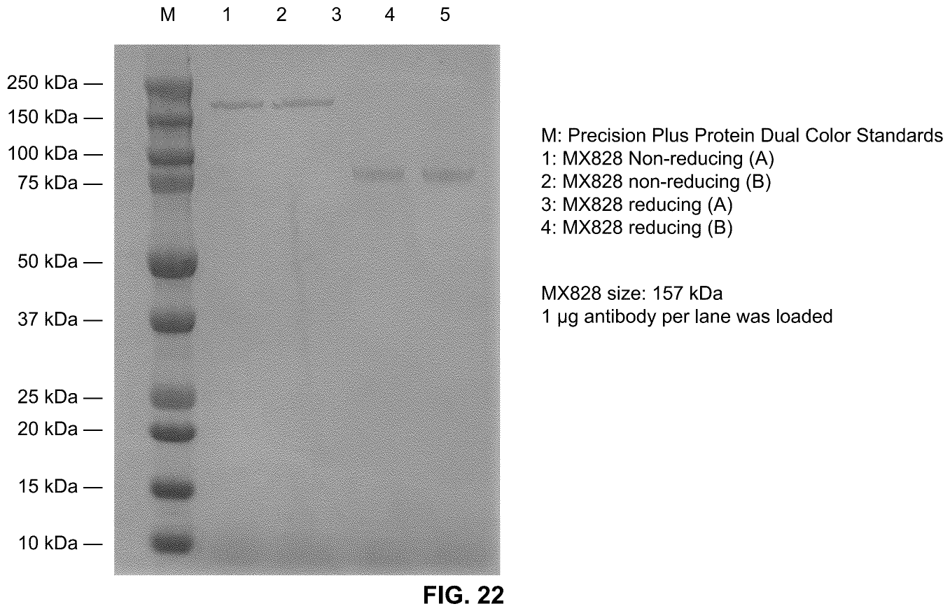

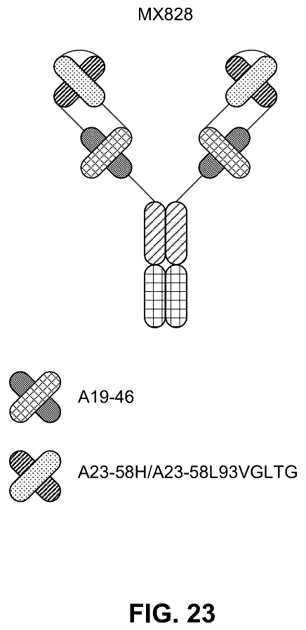

The in vitro production of the mRNAs encoding the antigen binding polypeptide complexes was tested. First, a representing candidate (MX828) was selected for the in vitro production of the mRNAs encoding the antigen binding polypeptide complexes. Specifically, the MD1186 plasmid encoding MX828 was digested as shown in FIG. 19 for restriction enzyme mapping. Then, the MD1186 plasmid was linearized with NotI restriction enzyme (FIG. 20), and used for mRNA in vitro transcription with standard methods. The resulting in vitro transcribed mRNA was run on an agarose gel for synthesis control (FIGS. 21A-21B). The in vitro transcribed mRNA was then transfected into Expi293 cells for in vitro production of the antibody, which resulted in a yield of 0.88-0.62 mg antibody after protein A purification (FIG. 22). A schematic of the structure of the produced test antigen binding polypeptide complex (MX828) is shown in FIG. 23.

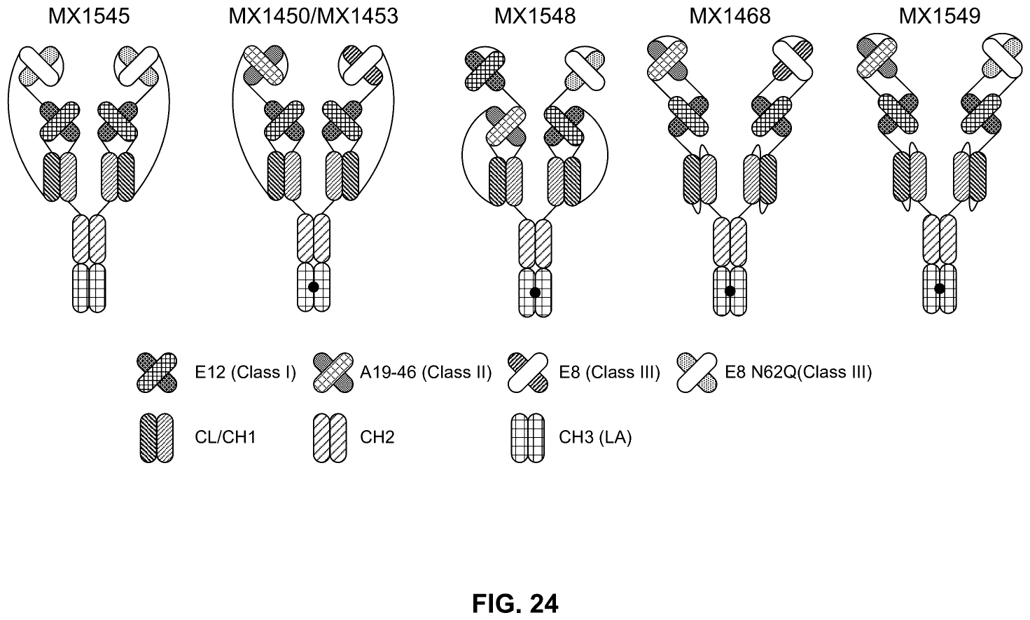

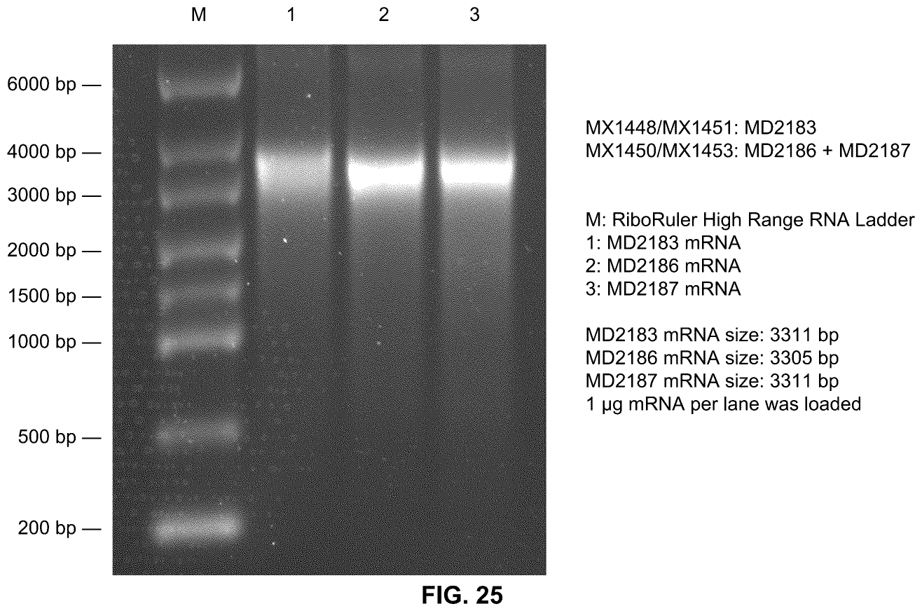

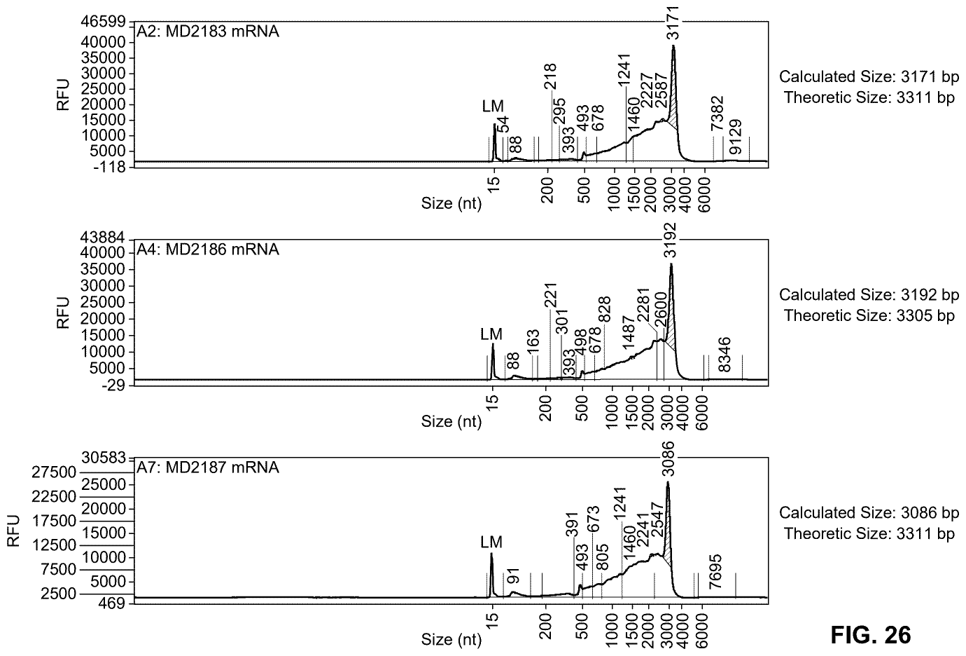

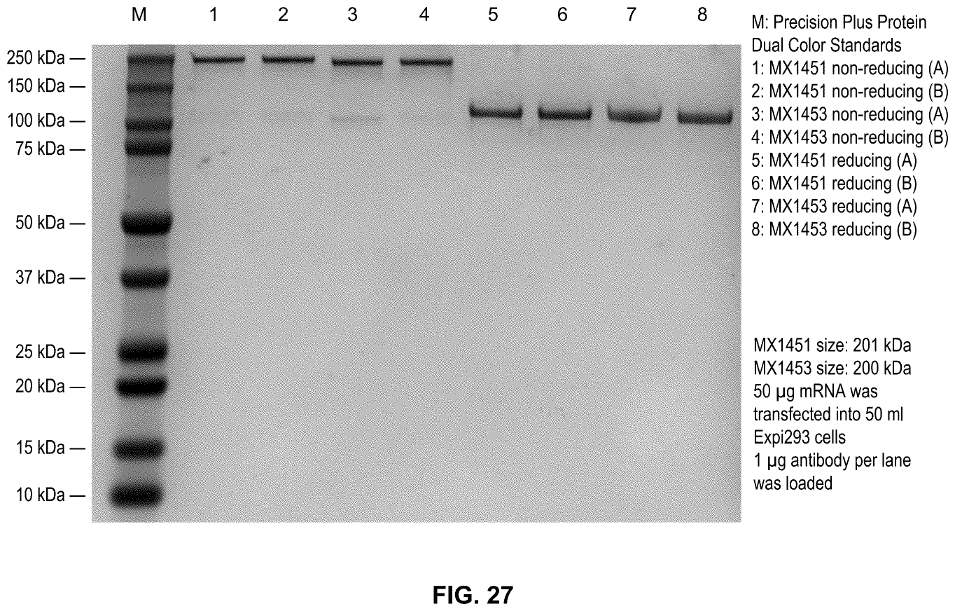

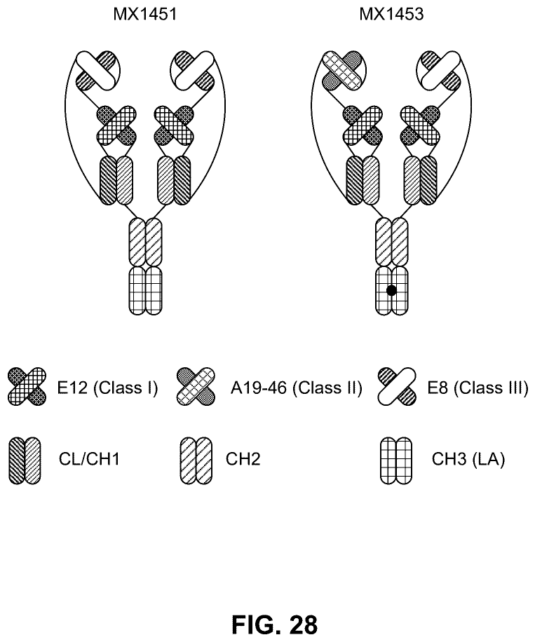

Next, selected additional candidates (MX1545, MX1450/MX1453, MX1548, MX1468, and MX1549) were also produced in vitro following the same procedure. The tested candidates comprise LA modifications and/or N62Q glycosylation modifications as indicated in the schematics of the structures of these candidates shown in FIG. 24. Among the candidates, MX1448/MX1451 and MX1450/MX1453 were selected for in vitro production. A gel electrophoresis of the in vitro synthesized mRNA is shown in FIG. 25, FIG. 26 shows a capillary gel electrophoresis by Fragment Analyzer of the produced mRNAs, and FIG. 27 shows a Western Blot analysis of the produced antigen binding polypeptide complexes, FIG. 28 shows the structures of the produced antigen binding polypeptide complexes.

### Example 11

### Optimization of Well-Performing Antigen Binding Polypeptide Complexes-1

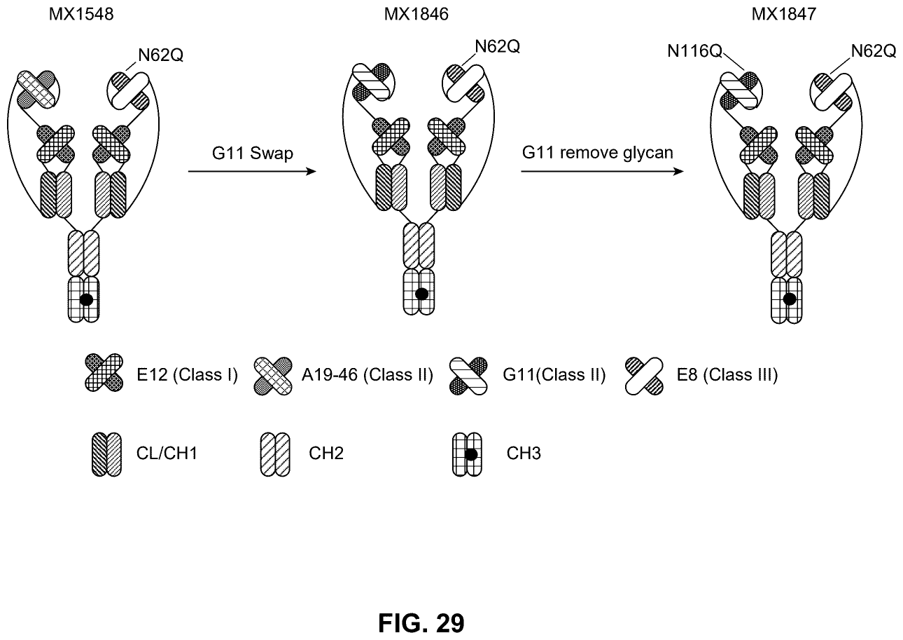

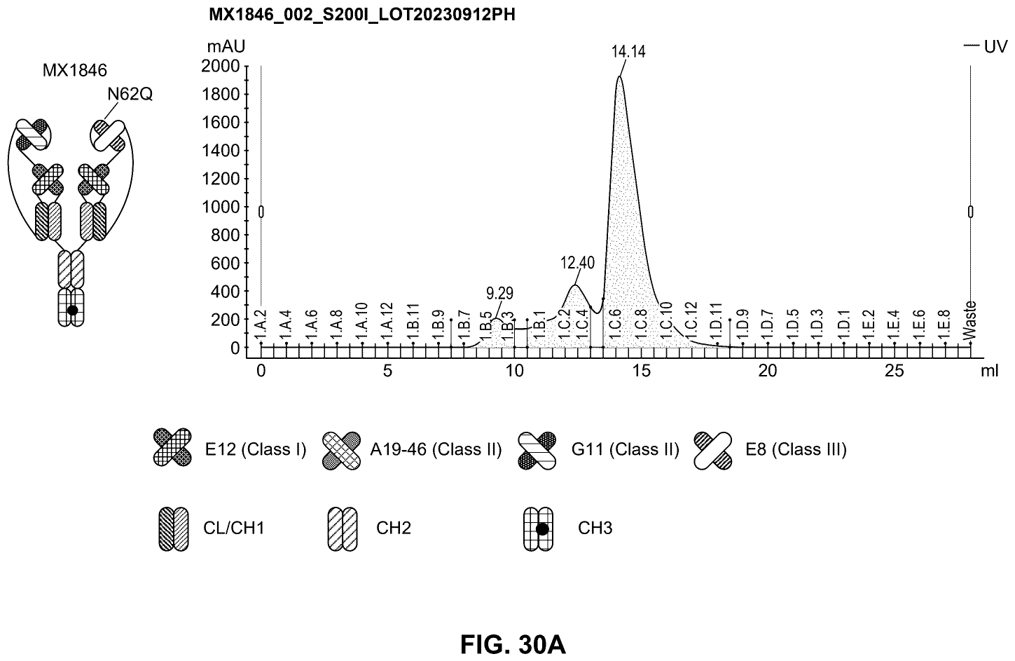

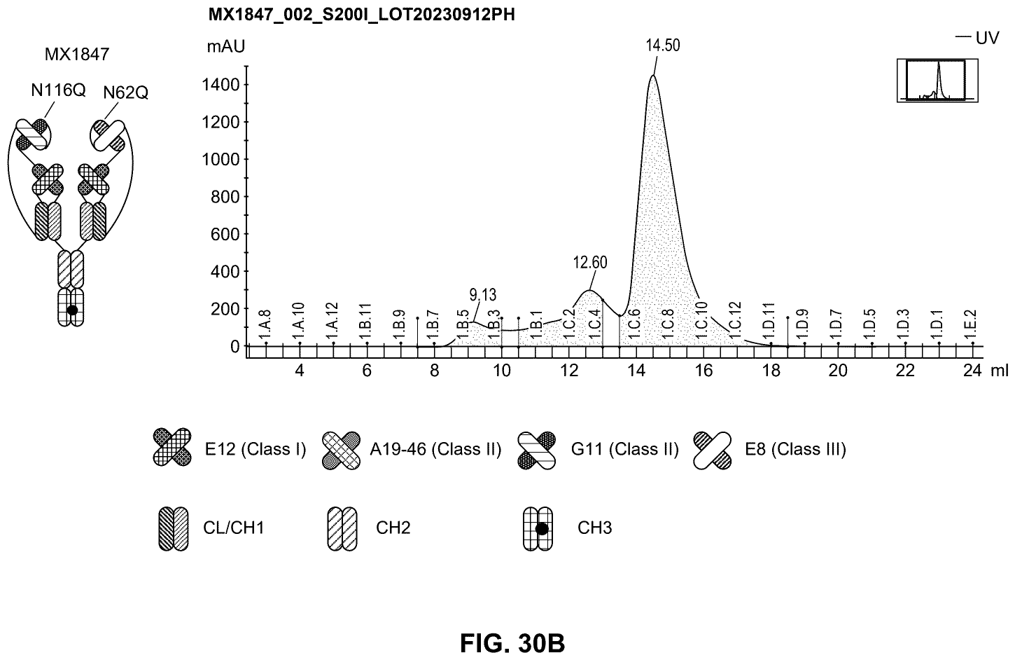

Well-performing antigen binding polypeptide complexes were further modified for optimization as follows. In MX1548, the VL and the VH of A19-46 were substituted with the VL and the VH of G11 to obtain MX1846. Further, MX1846 was modified to remove a glycosylation site on G11 (N116Q modification) to obtain MX1847. The above modifications to MX1548 (to obtain MX1846 and MX1847) are shown in FIG. 29. Both MX1846 and MX1847 (i.e., the de-glycosylated version of MX1846) were tested. Size exclusion chromatography (SEC) using superdex 200 increase column, and 10 mM Histidine pH 6.0 and 150 mM NaCl as running buffer, was performed for both MX1846 and MX1847. Both MX1846 and MX1847 were expressed in ExpiCHO-S culture and expression titers were measured by Octet BLI using protein A biosensor. Size exclusion chromatography profiles for both MX1846 and MX1847 are shown in FIGS. 30A-30B, and the expression titers for MX1548, MX1846 and MX1847 are shown in Table E9.

TABLE E9
MX1548, MX1846 and MX1847 expression titers.

| Name | Titer (mg/L) |
|---|---|
| MX1548 | 76.1 |
| MX1846 | 75.3 |
| MX1847 | 53.7 |

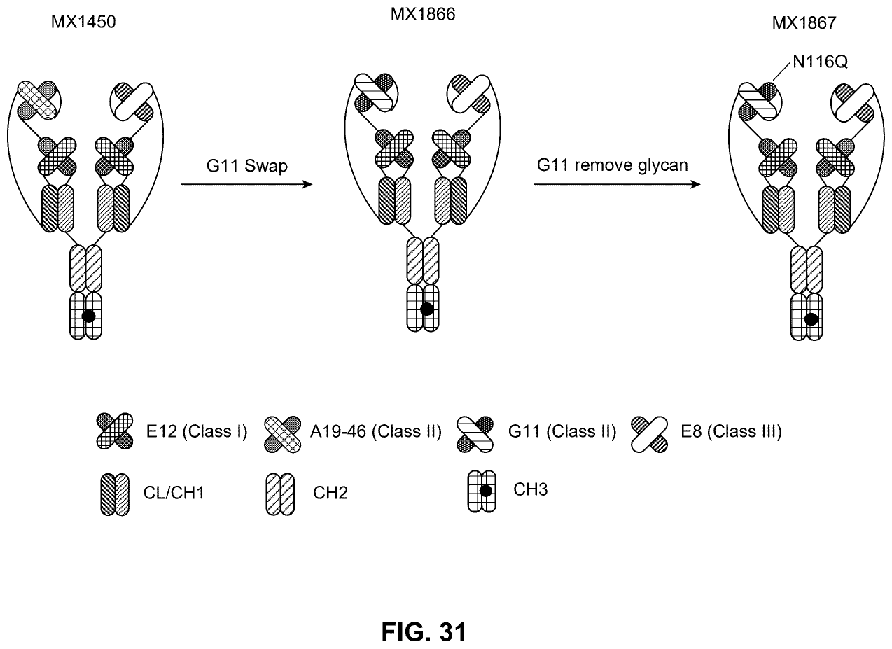

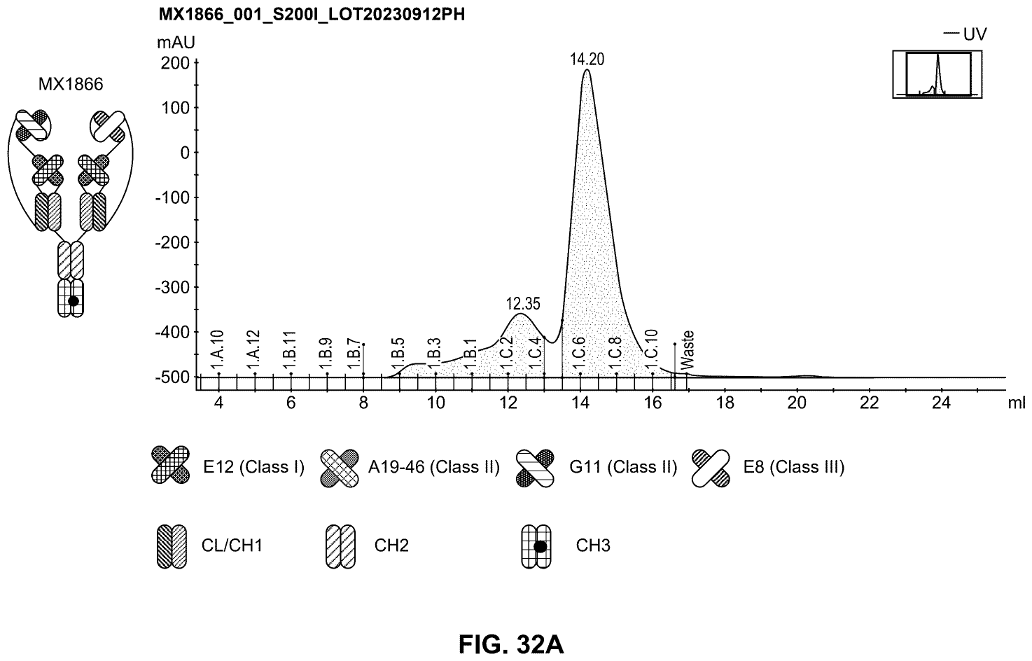

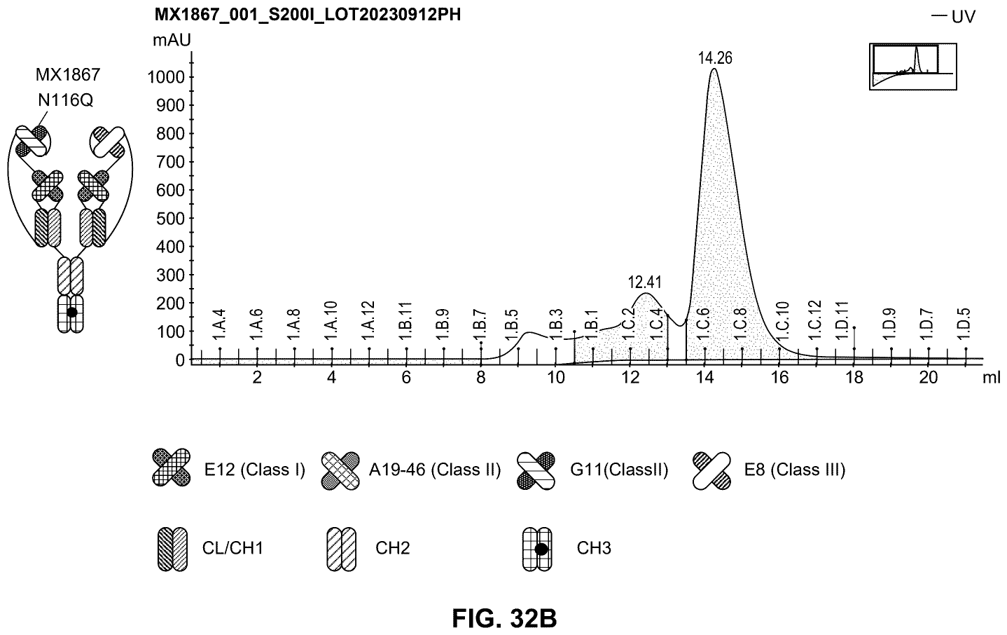

Similarly, in MX1450, the VL and the VH of A19-46 were substituted with the VL and the VH of G11 to obtain MX1866. Further, MX1866 was modified to remove a glycosylation site on G11 to obtain MX1867. The above modifications to MX1450 (to obtain MX1866 and MX1867) are shown in FIG. 31. Both MX1866 and MX1867 (i.e., the de-glycosylated version of MX1866) were tested. Size exclusion chromatography (SEC) using superdex 200 increase column, and 10 mM Histidine pH 6.0 and 150 mM NaCl as running buffer, was performed for both MX1866 and MX1867. Both MX1866 and MX1867 were expressed in ExpiCHO-S culture and expression titers were measured by Octet BLI using protein A biosensor. Size exclusion chromatography profiles for both MX1866 and MX1867 are shown in FIGS. 32A-32B, and the expression titers for MX1548, MX1866 and MX1867 are shown in Table E10.

TABLE E10
MX1548, MX1866 and MX1867 expression titers.

| Name | Titer (mg/L) |
|---|---|
| MX1548 | 76.1 |
| MX1866 | 42.3 |
| MX1867 | 53.8 |

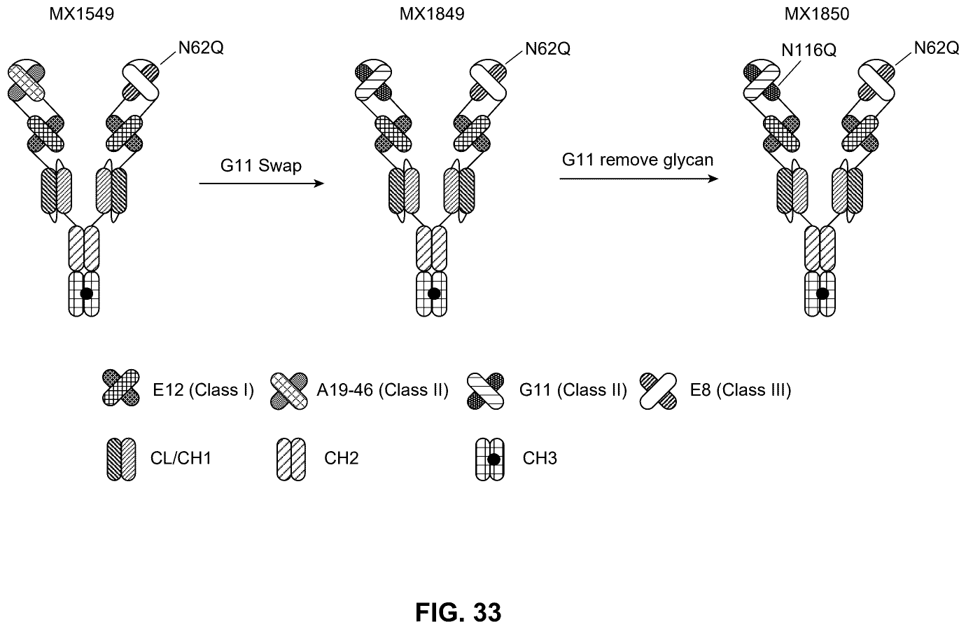

Further, in MX1549, the VL and the VH of A19-46 were substituted with the VL and the VH of G11 to obtain MX1849. Further, MX1849 was modified to remove a glycosylation site on G11 to obtain MX1850. The above modifications to MX1549 (to obtain MX1849 and MX1850) are shown in FIG. 33.

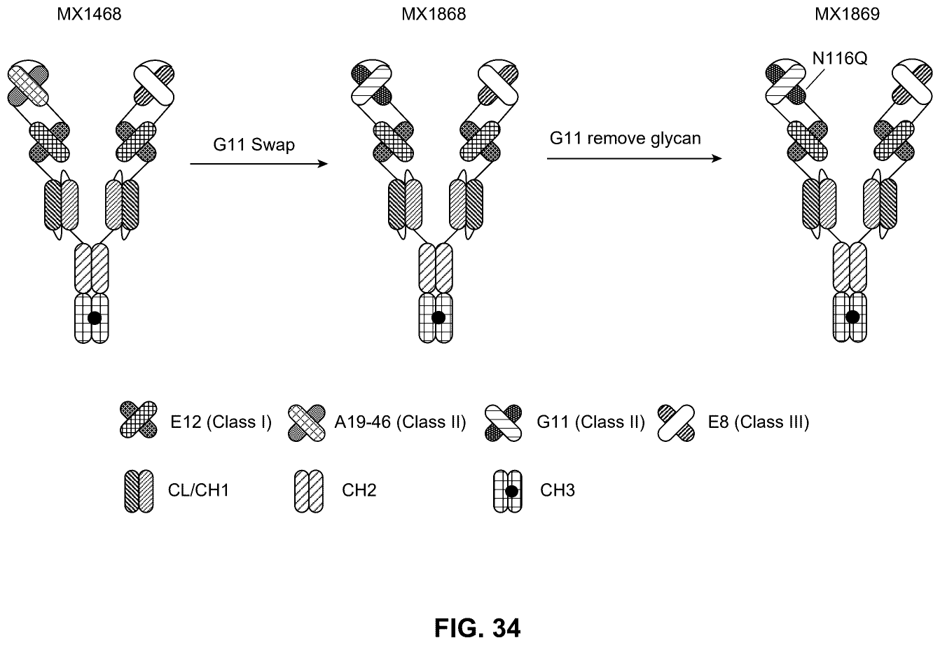

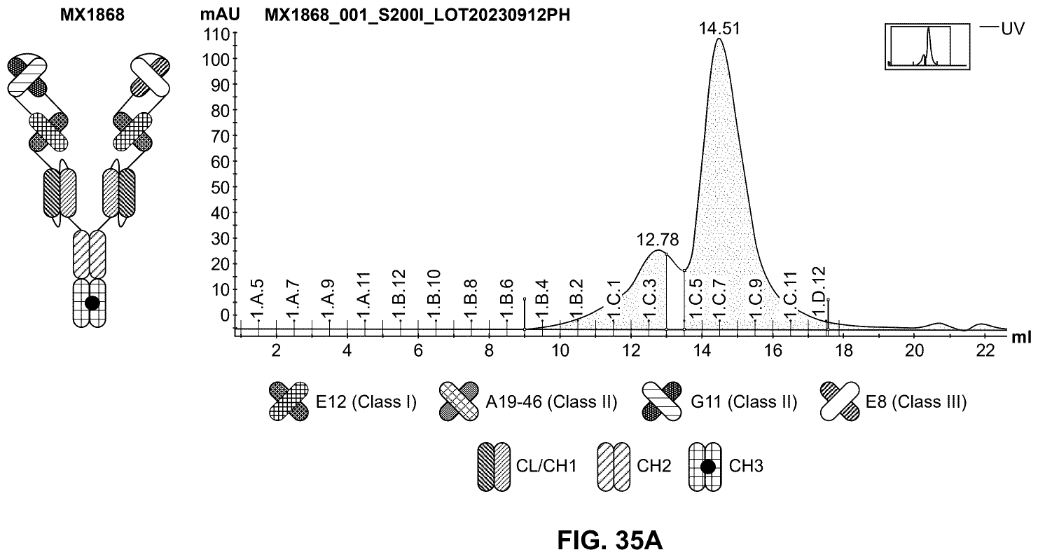

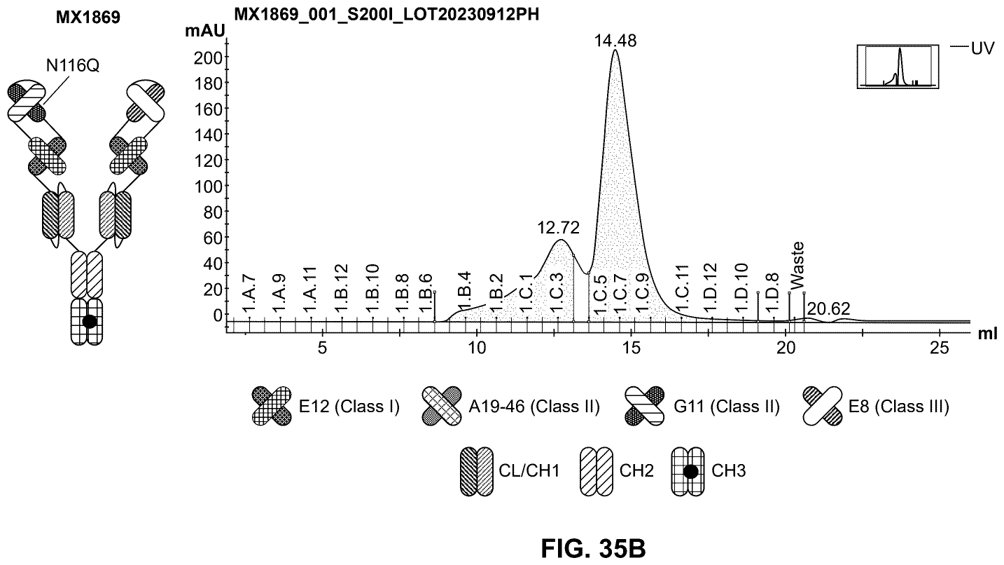

In addition, in MX1468, the VL and the VH of A19-46 were substituted with the VL and the VH of G11 to obtain MX1868. Further, MX1868 was modified to remove a glycosylation site on G11 to obtain MX1869. The above modifications to MX1468 (to obtain MX1868 and MX1869) are shown in FIG. 34. Both MX1868 and MX1869 (i.e., the de-glycosylated version of MX1868) were tested. Size exclusion chromatography (SEC) using superdex 200 increase column, and 10 mM Histidine pH 6.0 and 150 mM NaCl as running buffer, was performed for both MX1868 and MX1869. Both MX1868 and MX1869 were expressed in ExpiCHO-S culture and expression titers were measured by Octet BLI using protein A biosensor. Size exclusion chromatography profiles for both MX1868 and MX1869 are shown in FIGS. 35A-35B, and the expression titers for MX1549, MX1849, MX1850, MX1868, and MX1869 are shown in Table E11. * Of note, MX1869's titer was 12 mg/L, while MX1868's titer was 7.9 mg/L in another controlled experiment.

TABLE E11
MX1549, MX1850, MX1851, MX1868,
and MX1869 expression titers.

| Name | Titer (mg/L) |
|---|---|
| MX1549 | 85.3 |
| MX1849 | 106.7 |
| MX1850 | 112.1 |
| MX1868 | 76 |
| MX1869* | n/a |

The neutralizing activities of G11 swap molecules against SARS-CoV-2 variants described above was tested. Specifically, S-containing lentiviral pseudovirions were produced by co-transfection of packaging plasmid pCMVdR8.2, transducing plasmid pHR′ CMV-Luc, a TMPRSS2 plasmid and S plasmids from SARS-CoV-2 variants into 293T cells using Lipofectamine 3000 transfection reagent (L3000-001, ThermoFisher Scientific, Asheville, NC), 293T-ACE2 cells (provided by Dr. Michael Farzan) were plated into 96-well white/black Isoplates (PerkinElmer, Waltham, MA) at 7,500 cells per well the day before infection of SARS-CoV-2 pseudovirus. Serial dilutions of the anti-SARS-CoV-2 antigen binding polypeptide complexes and of the anti-SARS-CoV-2 monoclonal antibodies were mixed with titrated pseudoviruses, incubated for 45 minutes at 37° C., and added to cells in triplicate. Following 2 h of incubation, wells were replenished with 150 μl of fresh media. Cells were lysed 72 h later, and luciferase activity was measured with Microbeta (Perking Elmer). Percent neutralization and neutralization IC50 and IC80 values are shown in Table E12.

TABLE E12
pseudotyped neutralization potency (IC50 and IC80) for the tested anti-SARS-CoV-2 antigen
binding polypeptide complexes (WT : Wild type. G11 NQ : G11 HC N116Q. E8 NQ : E8 HC N62Q G11
or E8 NQ mutation made to remove the glycosylation site on the corresponding binder-AZD3152 is
AstraZeneca's investigational long-acting COVID-19 antibody, used here as benchmark) (IC50 and IC80
are expressed in nM, N.T.: not tested).

| MSTAR | | | Anti- | WA.1 | | BA.2 | | BA.4/5 | | BQ.1.1 | | |
|---|---|---|---|---|---|---|---|---|---|---|---|---|
| version | Class 2 | Class 3 | body | IC50 | IC80 | IC50 | IC80 | IC50 | IC80 | IC50 | IC80 | |
| V1 | A19-46 | E8 NQ | MX1548 | 0.0908 | 0.1417 | 0.0196 | 0.0581 | 0.0496 | 0.1616 | 0.0106 | 0.0906 | |
| | G11 WT | E8 NQ | MX1846 | 0.2073 | 0.6850 | 0.0228 | 0.0808 | 0.0337 | 0.1021 | 0.0369 | 0.0925 | |
| | G11 NQ | E8 NQ | MX1847 | 0.0834 | 0.2638 | 0.0208 | 0.0662 | 0.0467 | 0.1095 | 0.0375 | 0.1151 | |
| | G11 WT | E8 WT | MX1866 | 0.0402 | 0.2272 | 0.0225 | 0.1055 | 0.0150 | 0.0653 | 0.0157 | 0.0703 | |
| | G11 NQ | E8 WT | MX1867 | 0.0791 | 0.2205 | 0.0385 | 0.0961 | 0.0153 | 0.0578 | 0.0113 | 0.0491 | |
| V2 | A19-46 | E8 WT | MX1468 | 0.0309 | 0.1676 | 0.0306 | 0.0983 | 0.0591 | 0.1640 | 0.0254 | 0.0988 | |
| | G11 WT | E8 WT | MX1868 | 0.0378 | 0.2548 | 0.0221 | 0.0918 | 0.0759 | 0.1863 | 0.0188 | 0.0758 | |
| | G11 NQ | E8 WT | MX1869 | 0.0595 | 0.1976 | 0.0281 | 0.0817 | 0.0523 | 0.1325 | 0.0254 | 0.1058 | |
| | G11 WT | E8 NQ | MX1849 | 0.0875 | 0.6850 | 0.0225 | 0.0798 | 0.0514 | 0.1701 | 0.0389 | 0.1314 | |
| | G11 NQ | E8 NQ | MX1850 | 0.1119 | 0.1699 | 0.0204 | 0.0479 | 0.0303 | 0.0796 | 0.0211 | 0.1161 | |
| MSTAR | | | Anti- | XBB.1.5 | | EG.5 | | XBB.2.3.2 | | BA.2.86 | | |
| version | Class 2 | Class 3 | body | IC50 | IC80 | IC50 | IC80 | IC50 | IC80 | IC50 | IC80 | |
| V1 | A19-46 | E8 NQ | MX1548 | 0.1028 | 0.2993 | 0.3285 | 1.2018 | 0.0510 | 0.2000 | 0.1000 | 0.3700 | |
| | G11 WT | E8 NQ | MX1846 | 0.0995 | 0.2208 | 0.0696 | 0.2371 | 0.0160 | 0.0720 | 0.0860 | 0.3200 | |
| | G11 NQ | E8 NQ | MX1847 | 0.1217 | 0.2593 | 0.1295 | 0.3380 | 0.0210 | 0.0700 | 0.1300 | 0.3900 | |
| | G11 WT | E8 WT | MX1866 | N.T. | N.T. | 0.3400 | 0.5208 | N.T. | N.T. | N.T. | N.T. | |
| | G11 NQ | E8 WT | MX1867 | N.T. | N.T. | 0.1855 | 0.2688 | N.T. | N.T. | N.T. | N.T. | |
| V2 | A19-46 | E8 WT | MX1468 | N.T. | N.T. | 0.0195 | 0.0445 | 0.0140 | 0.0450 | 0.0370 | 0.1400 | |
| | G11 WT | E8 WT | MX1868 | N.T. | N.T. | 0.1609 | 0.1799 | N.T. | N.T. | N.T. | N.T. | |
| | G11 NQ | E8 WT | MX1869 | N.T. | N.T. | 0.1032 | 0.2042 | N.T. | N.T. | N.T. | N.T. | |
| | G11 WT | E8 NQ | MX1849 | 0.1067 | 0.2137 | 0.0404 | 0.1761 | 0.1600 | 0.4600 | 0.6900 | 1.8000 | |
| | G11 NQ | E8 NQ | MX1850 | 0.0943 | 0.1373 | 0.0339 | 0.1367 | 0.0370 | 0.1600 | 0.4100 | 1.3000 | |
| Speci- | | | D614G | | B.1.351 | | B.1.617.2 | | | | | |
| ficity | Antibody | Class | IC50 | IC80 | IC50 | IC80 | IC50 | | | IC80 | | |
| Tri- | MX1548 | I + II + | 0.0100 | 0.0220 | 0.0070 | 0.0150 | 0.1130 | | | 0.1240 | | |
| specific | | III | | | | | | | | | | |
| Tri- | MX1450 | I + II + | 0.0190 | 0.0450 | 0.0270 | 0.0710 | 0.1280 | | | 0.2110 | | |
| specific | | III | | | | | | | | | | |
| Tri- | MX1846 | I + II + | 0.2073 | 0.6850 | N.T. | N.T. | N.T. | | | N.T. | | |
| specific | | III | | | | | | | | | | |
| Tri- | MX1847 | I + II + | 0.0834 | 0.2638 | N.T. | N.T. | N.T. | | | N.T. | | |
| specific | | III | | | | | | | | | | |
| Mono- | E12 | I | 0.0020 | 0.0350 | 0.0180 | 0.0330 | 0.1130 | | | 0.2060 | | |
| clonal | | | | | | | | | | | | |
| Mono- | E8 | III | 0.0060 | 0.0150 | 0.0100 | 0.0180 | 0.0110 | | | 0.0210 | | |
| clonal | | | | | | | | | | | | |
| Mono- | A19-46 | II | 0.0710 | 0.2500 | 0.2580 | 0.8060 | >66.7 | | | >66.7 | | |
| clonal | | | | | | | | | | | | |
| Mono- | G11 | II | 0.0141 | 0.0279 | N.T. | N.T. | N.T. | | | N.T. | | |
| clonal | | | | | | | | | | | | |
| Mono- | AZD3152 | I | 0.0160 | 0.0320 | 0.0230 | 0.0410 | 0.2810 | | | 0.5180 | | |
| clonal | | | | | | | | | | | | |
| Speci- | | | BA.1 | | BA.2 | | BA.4/5 | | BQ.1.1 | | | |
| ficity | Antibody | Class | IC50 | IC80 | IC50 | IC80 | IC50 | IC80 | IC50 | IC80 | | |
| Tri- | MX1548 | I + II + | 0.0210 | 0.0440 | N.T. | N.T. | 0.0310 | 0.0710 | 0.1370 | 0.4060 | | |
| specific | | III | | | | | | | | | | |
| Tri- | MX1450 | I + II + | 0.0190 | 0.0410 | N.T. | N.T. | 0.0540 | 0.1150 | 0.1510 | 0.4370 | | |
| specific | | III | | | | | | | | | | |
| Tri- | MX1846 | I + II + | N.T. | N.T. | 0.0228 | 0.0808 | 0.0337 | 0.1021 | 0.0369 | 0.0925 | | |
| specific | | III | | | | | | | | | | |
| Tri- | MX1847 | I + II + | N.T. | N.T. | 0.0208 | 0.0662 | 0.0467 | 0.1095 | 0.0375 | 0.1151 | | |
| specific | | III | | | | | | | | | | |
| Mono- | E12 | I | 0.0430 | 0.0970 | N.T. | N.T. | 0.0210 | 0.0570 | 0.0740 | 0.2540 | | |
| clonal | | | | | | | | | | | | |
| Mono- | E8 | III | 0.2340 | 0.5290 | N.T. | N.T. | 0.0090 | 0.0180 | 2.8700 | 12.0600 | | |
| clonal | | | | | | | | | | | | |
| Mono- | A19-46 | II | 0.3120 | 0.8630 | N.T. | N.T. | >66.7 | >66.7 | >66.7 | >66.7 | | |
| clonal | | | | | | | | | | | | |
| Mono- | G11 | II | 0.0301 | 0.1136 | N.T. | N.T. | 0.0140 | 0.0686 | 0.8742 | 2.4886 | | |
| clonal | | | | | | | | | | | | |
| Mono- | AZD3152 | I | 0.1230 | 0.3130 | N.T. | N.T. | 0.1020 | 0.2660 | 0.3920 | 0.9440 | | |
| clonal | | | | | | | | | | | | |
| Speci- | | | XBB.1 | | XBB.1.5 | | XBB.1.16 | | CH.1.1 | | XBC.1 | |
| ficity | Antibody | Class | IC50 | IC80 | IC50 | IC80 | IC50 | IC80 | IC50 | IC80 | IC50 | IC80 |
| Tri- | MX1548 | I + II + | 0.1070 | 0.2270 | 0.0720 | 0.1740 | 0.0850 | 0.2120 | 0.1940 | 0.8050 | 0.0100 | 0.0250 |
| specific | | III | | | | | | | | | | |
| Tri- | MX1450 | I + II + | 0.1040 | 0.3130 | 0.1190 | 0.2420 | 0.0840 | 0.3310 | 0.3550 | 0.9200 | 0.0030 | 0.0140 |
| specific | | III | | | | | | | | | | |
| Tri- | MX1846 | I + II + | N.T. | N.T. | 0.0995 | 0.2208 | N.T. | N.T. | N.T. | N.T. | N.T. | N.T. |
| specific | | III | | | | | | | | | | |
| Tri- | MX1847 | I + II + | N.T. | N.T. | 0.1217 | 0.2593 | N.T. | N.T. | N.T. | N.T. | N.T. | N.T. |
| specific | | III | | | | | | | | | | |
| Mono- | E12 | I | 0.0990 | 0.3040 | 0.1130 | 0.2350 | 0.0160 | 0.1700 | 0.0450 | 0.4420 | 0.0075 | 0.0895 |
| clonal | | | | | | | | | | | | |
| Mono- | A19-46 | II | >66.7 | >66.7 | >66.7 | >66.7 | >66.7 | >66.7 | N.T. | N.T. | 15.6200 | >66.7 |
| clonal | | | | | | | | | | | | |
| Mono- | G11 | II | 0.0584 | 0.1710 | 0.0860 | 0.3151 | 0.1027 | 0.3083 | N.T. | N.T. | 0.0168 | 0.0469 |
| clonal | E8 | III | 1.4900 | 19.5000 | 1.6900 | 13.1200 | 3.4100 | 17.3300 | N.T. | N.T. | 0.0108 | 0.0527 |
| Mono- | | | | | | | | | | | | |
| clonal | B2 | III | N.T. | N.T. | N.T. | N.T. | N.T. | N.T. | N.T. | N.T. | N.T. | N.T. |
| Mono- | | | | | | | | | | | | |
| clonal | A18- | I/IV | N.T. | N.T. | N.T. | N.T. | N.T. | N.T. | N.T. | N.T. | N.T. | N.T. |
| Mono- | 448.1 | | | | | | | | | | | |
| clonal | AZD3152 | I | 0.0960 | 0.3020 | 0.1200 | 0.3080 | 0.0920 | 0.3810 | 0.5010 | 1.4710 | 0.0322 | 0.2095 |
| Mono- | | | | | | | | | | | | |
| Speci- | | | XBB.2.3.2 | | EG.5.1 | | BA.2.86 | | XBB.1.16.6 | | FL.1.5.1 | |
| ficity | Antibody | Class | IC50 | IC80 | IC50 | IC80 | IC50 | IC80 | IC50 | IC80 | IC50 | IC80 |
| Tri- | MX1548 | I + II + | 0.1200 | 0.2130 | 0.1600 | 0.5700 | 0.1000 | 0.3700 | 0.2874 | 0.8466 | 0.6478 | 1.5586 |
| specific | | III | | | | | | | | | | |
| Tri- | MX1450 | I + II + | 0.1840 | 0.5030 | 0.1600 | 0.5200 | 0.1400 | 0.4000 | 1.1683 | 3.3616 | 0.6925 | 4.7157 |
| specific | | III | | | | | | | | | | |
| Tri- | MX1846 | I + II + | 0.0160 | 0.0720 | 0.0700 | 0.2400 | 0.0860 | 0.3200 | 0.0472 | 0.1361 | 0.0873 | 0.2480 |
| specific | | III | | | | | | | | | | |
| Tri- | MX1847 | I + II + | 0.0210 | 0.0700 | 0.1300 | 0.3400 | 0.1300 | 0.3900 | 0.0380 | 0.1681 | 0.0787 | 0.2470 |
| specific | | III | | | | | | | | | | |
| Mono- | E12 | I | 0.2380 | 0.7666 | 1.3580 | 2.8970 | 0.4200 | 0.7100 | 2.3580 | 7.0980 | 2.3190 | 6.5310 |
| clonal | | | | | | | | | | | | |
| Mono- | A19-46 | II | >66.7 | >66.7 | >66.7 | >66.7 | >66.7 | >66.7 | >66.7 | >66.7 | >66.7 | >66.7 |
| clonal | | | | | | | | | | | | |
| Mono- | G11 | II | 0.2129 | 0.5816 | 0.7351 | 1.9083 | >66.7 | >66.7 | 0.3917 | 0.7912 | 0.9549 | 2.4525 |
| clonal | | | | | | | | | | | | |
| Mono- | E8 | III | 0.3423 | >66.7 | 0.5598 | 7.0722 | >66.7 | >66.7 | 0.9680 | 10.2100 | 0.5781 | 5.6570 |
| clonal | | | | | | | | | | | | |
| Mono- | B2 | III | N.T. | N.T. | 0.4452 | 1.1031 | 0.3630 | 0.9549 | 0.7516 | 2.1506 | 0.9102 | 2.4031 |
| clonal | | | | | | | | | | | | |
| Mono- | A18- | I/IV | N.T. | N.T. | 0.0176 | 0.0670 | 0.0150 | 0.0715 | 0.0233 | 0.0652 | 0.0232 | 0.0694 |
| clonal | 448.1 | | | | | | | | | | | |
| Mono- | AZD3152 | I | 0.0806 | 0.4880 | >66.7 | >66.7 | 1.8000 | 5.4000 | >66.7 | >66.7 | >66.7 | >66.7 |
| clonal | | | | | | | | | | | | |

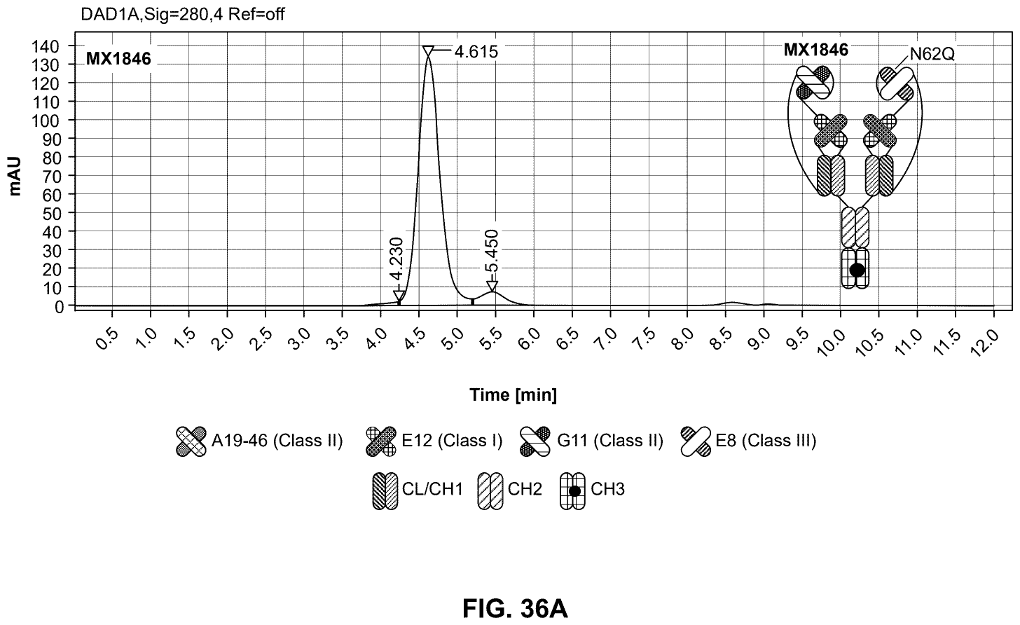

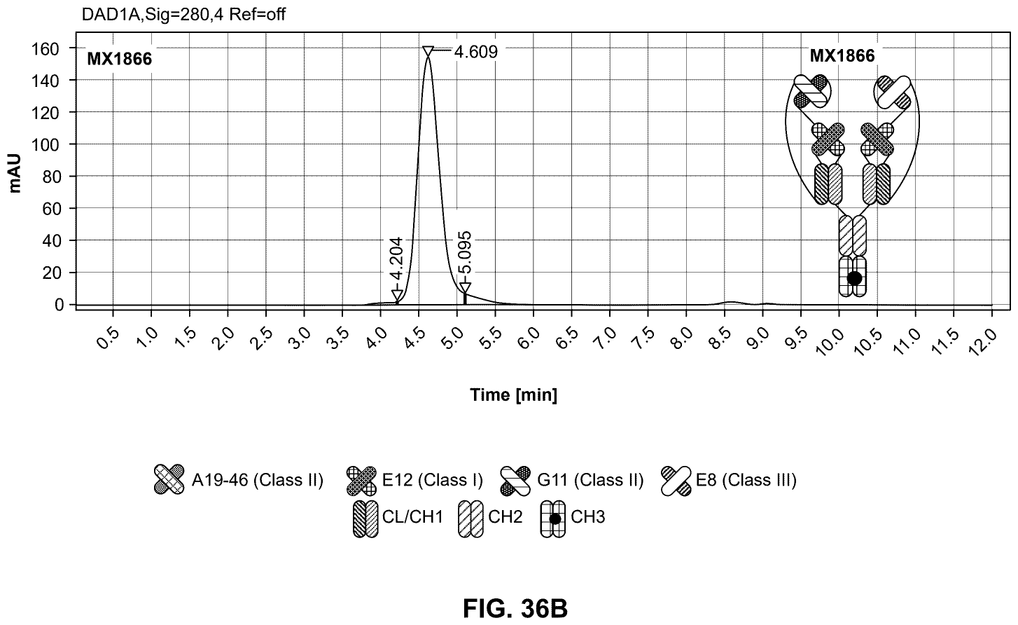

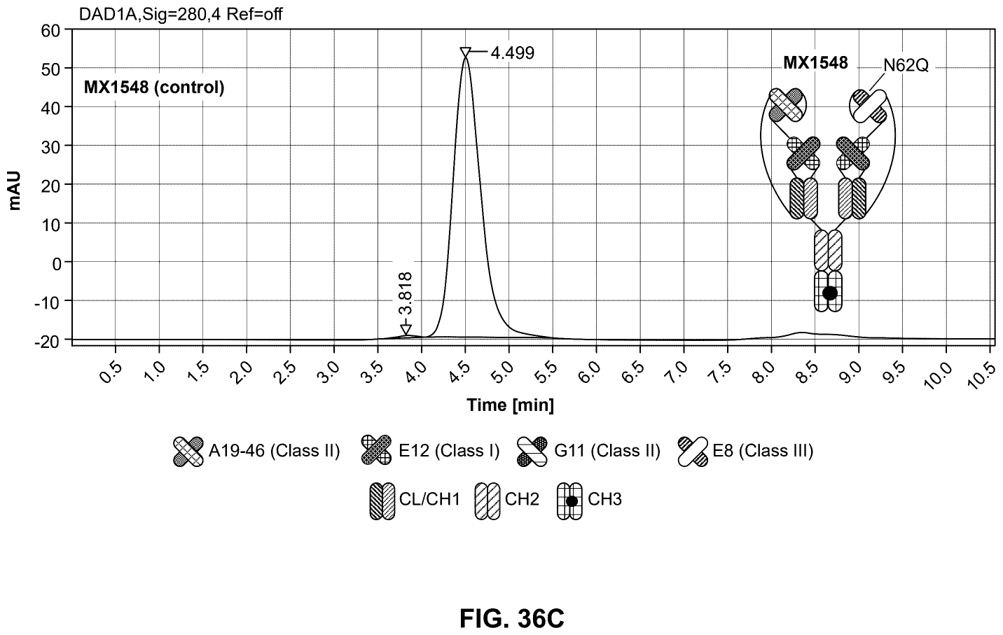

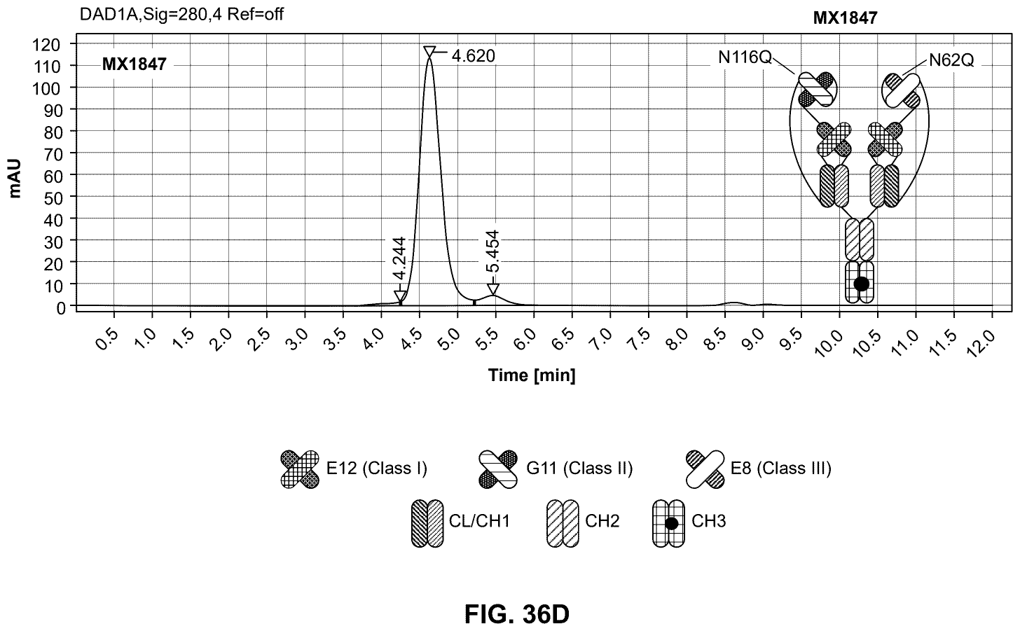

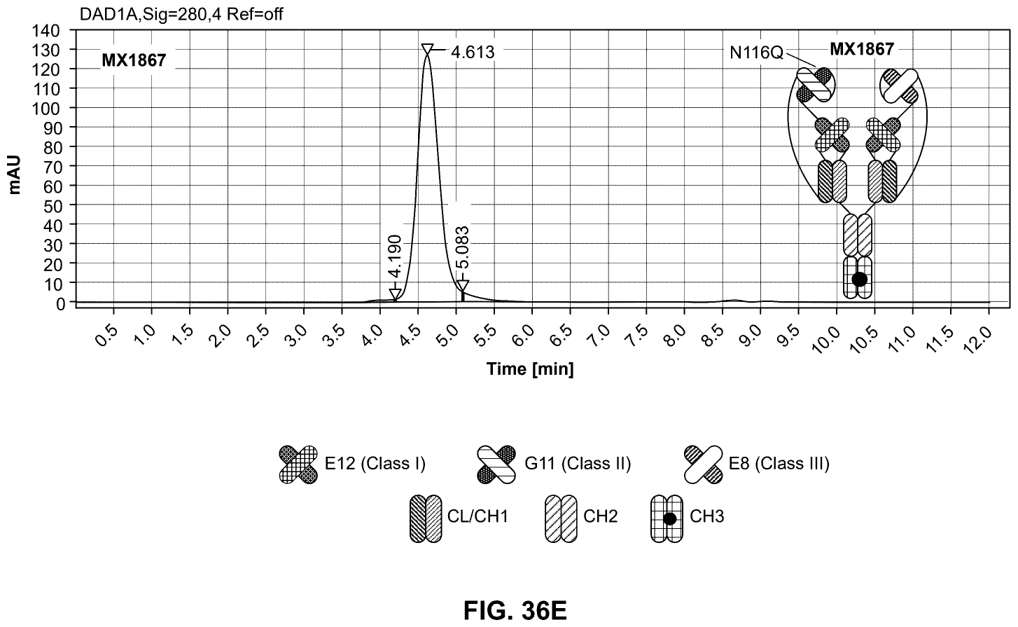

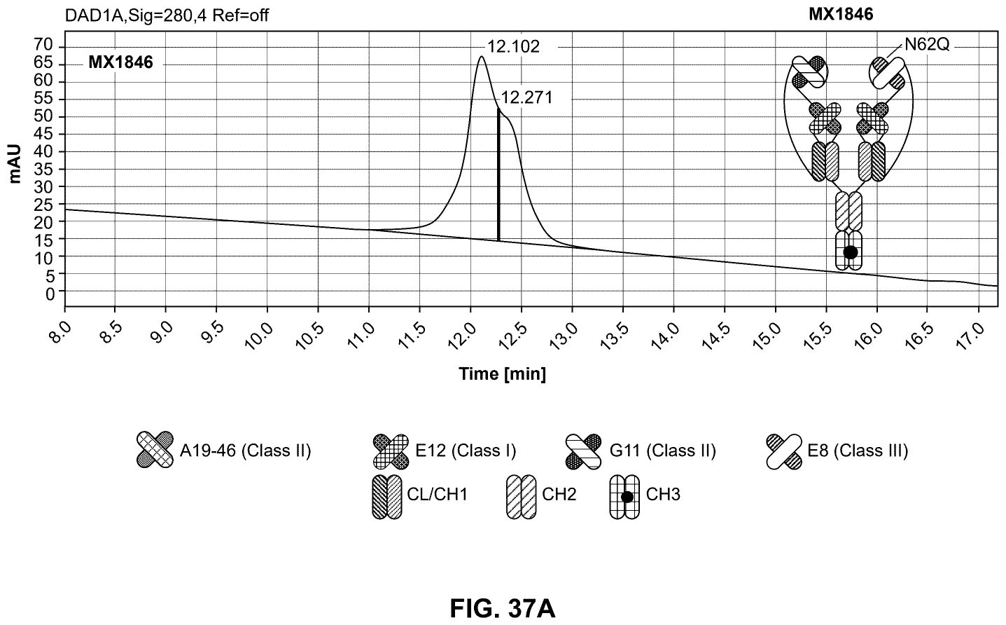

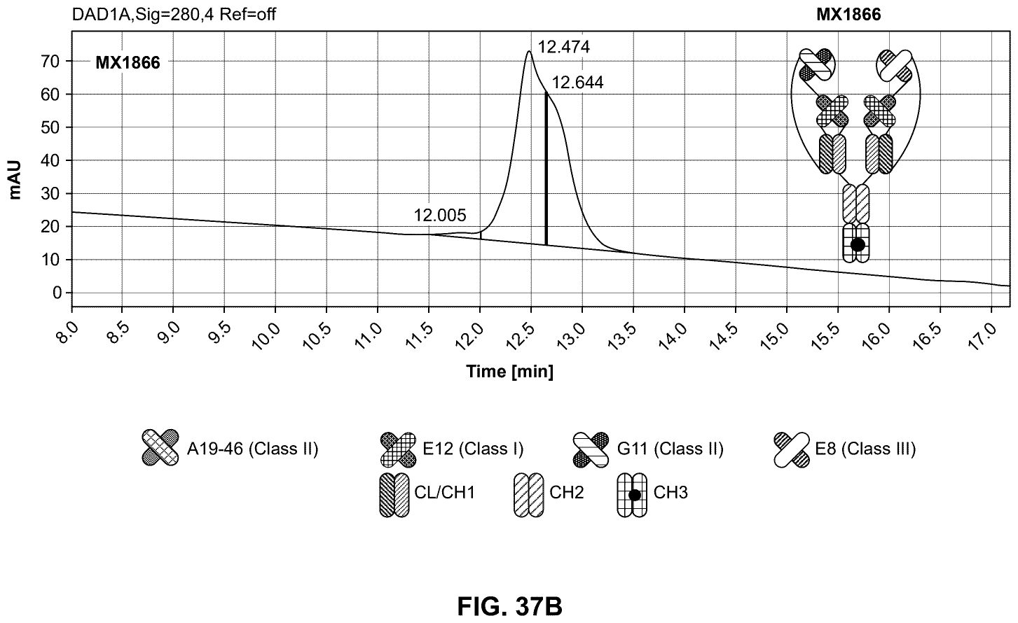

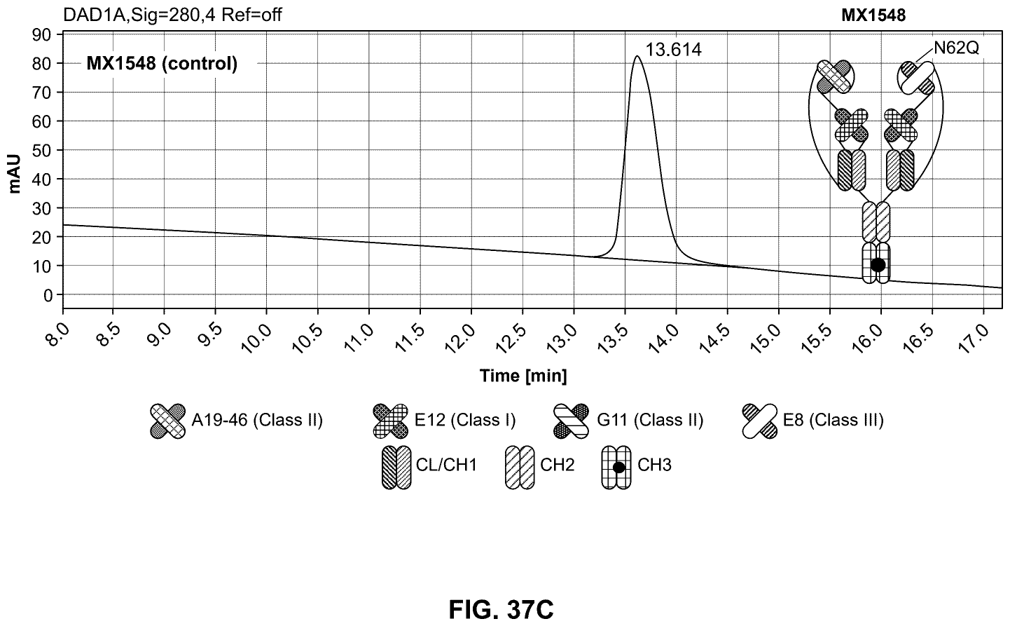

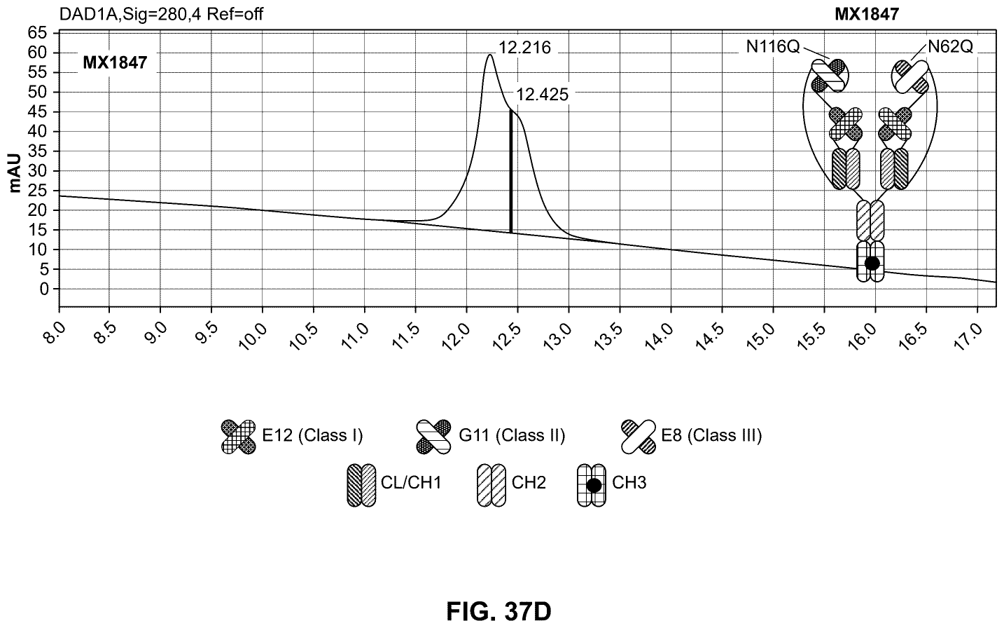

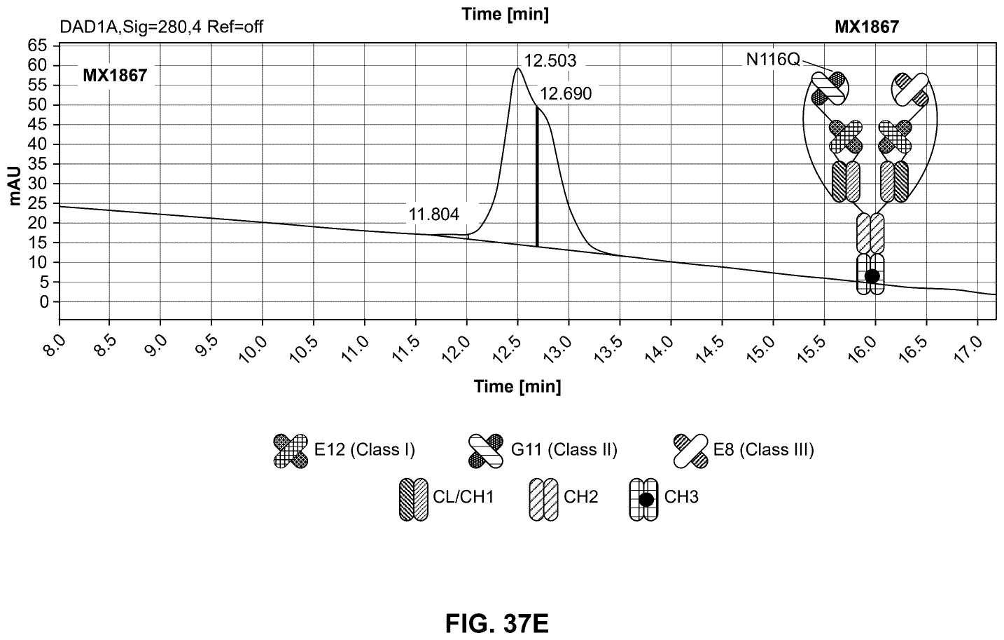

HPLC-analytical SEC profiles were obtained for MX1846, MX1847, MX1866, MX1867, and for MX1548 as control; and are shown in FIGS. 36A-36E. Further, HIC profiles were obtained for MX1846, MX1847, MX1866, MX1867, and for MX1548 as control; and are shown in FIGS. 37A-37E.

Table E13: shows comparative data for MX1846, MX1847, MX1866, MX1867, MX1868, and MX1849.

TABLE E13
Comparative data for MX1846, MX1847, MX1866, MX1867, MX1868, and MX1849.

| ID | MX1846 | MX1847 | MX1866 | MX1867 | MX1868 | MX1849 |
|---|---|---|---|---|---|---|
| Specificity | trispecific | trispecific | trispecific | trispecific | trispecific | trispecific |
| Format | MSTAR | MSTAR | MSTAR | MSTAR | MSTAR | MSTAR |
| | CL/CH1 v1 | CL/CH1 v1 | CL/CH1 v1 | CL/CH1 v1 | CL/CH1 | CL/CH1 |
| | | | | | v2 | v2 |
| Half-Life | LA | LA | LA | LA | LA | LA |
| Extension | | | | | | |
| Mutation (Fc) | | | | | | |
| E8 Heavy Chain | N62Q | N62Q | WT | WT | WT | N62Q |
| G11 Heavy Chain | WT | N116Q | WT | N116Q | WT | WT |
| Titer | 62 mg/L | 41 mg/L | 42 mg/L | 54 mg/L | 35 mg/L | 38 mg/L |
| Neutralization | highly | highly | highly | highly | highly | highly |
| Profile | potent (<1 | potent (<1 | potent (<1 | potent (<1 | potent (<1 | potent (<1 |
| | nM) | nM) | nM) | nM) | nM) | nM) |
| Binding to RBD | positive BLI | positive BLI | positive BLI | positive BLI | N.T | N.T |
| | binding to | binding to | binding to | binding to | | |
| | COVID | COVID | COVID | COVID | | |
| | RBD | RBD | RBD | RBD | | |
| Purity (aSEC) | 96.5% | 94.8% | 96.2% | 96.6% | 85.5% | 87.5% |
| Purity (aHIC) | homogenous | homogenous | homogenous | homogenous | N.T | N.T |
| | and relative | and relative | and relative | and relative | | |
| | short | short | short | short | | |
| | retention | retention | retention | retention | | |
| | time | time | time | time | | |
| Experimental pI | 8.642 | 8.633 | 8.643 | 8.633 | N.T | N.T |
| (cIEF) | | | | | | |
| Solubility | ~94 mg/ml | N.T | N.T | N.T | N.T | N.T |
| Freeze/Thaw | Minor | Minor | Minor | Minor | N.T | N.T |
| Stability (5 Cycles) | aggregation | aggregation | aggregation | aggregation | | |
| | after 5 | after 5 | after 5 | after 5 | | |
| | cycles, | cycles, | cycles, | cycles, | | |
| | improved | improved | improved | improved | | |
| | after adding | after adding | after adding | after adding | | |
| | PS80 | PS80 | PS80 | PS80 | | |
| Thermostability | 1 week | 1 week | 1 week | 1 week | N.T | N.T |
| | good, 2 | good, 2 | good, 2 | good, 2 | | |
| | weeks on | weeks on | weeks on | weeks on | | |
| | going | going | going | going | | |
| pH Stability (pH | good | good | good | good | N.T | N.T |
| 3.5 and pH 9.0) | | | | | | |
| Accelerated | Little to no | Little to no | Little to no | Little to no | N.T | N.T |
| Degradation (pH | degradation | degradation | degradation | degradation | | |
| 6.0, 40° C.) | seen in | seen in | seen in | seen in | | |
| | aSEC, CE- | aSEC, CE- | aSEC, CE- | aSEC, CE- | | |
| | SDS, or BLI | SDS, or BLI | SDS, or BLI | SDS, or BLI | | |
| | binding | binding | binding | binding | | |
| Serum Stability | Stable in | Stable in | Stable in | Stable in | N.T | N.T |
| | serum-no | serum-no | serum-no | serum-no | | |
| | change to | change to | change to | change to | | |
| | aSEC or | aSEC or | aSEC or | aSEC or | | |
| | BLI binding | BLI binding | BLI binding | BLI binding | | |
| Platelet Binding | No platelet | N.T | N.T | N.T | N.T | N.T |
| | binding | | | | | |

### Example 12

### Tetravalent Tetraspecific Pan-Sarbecovirus Antigen Binding Constructs

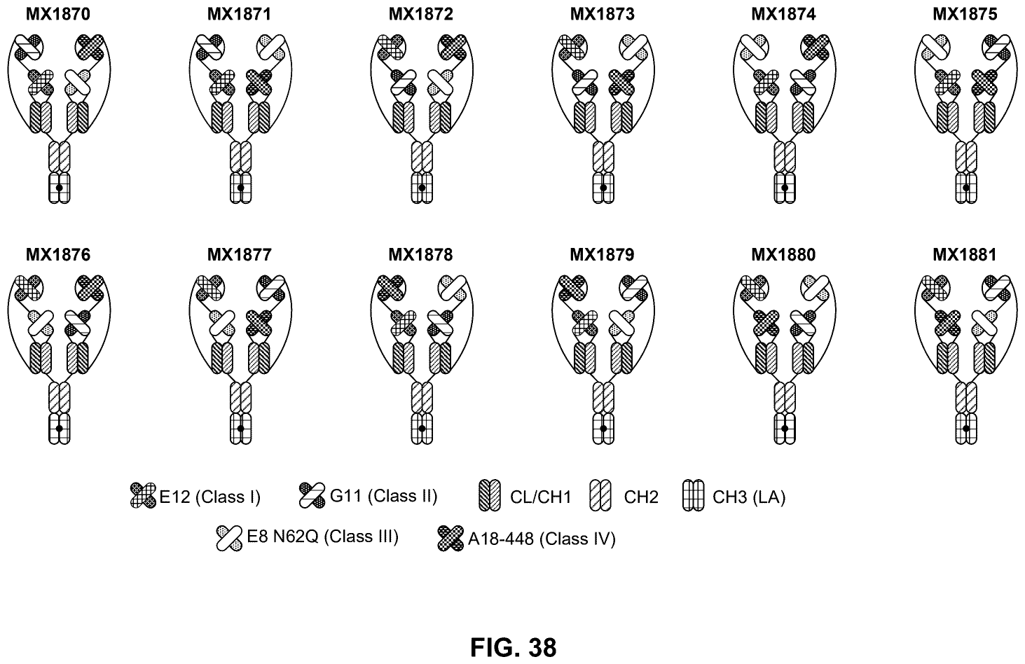

The tetravalent tetraspecific pan-sarbecovirus antibodies shown in FIG. 38 were produced. ExpiCHO cells were transfected using a standard protocol and were then harvested at day 5. The results of the production of the tetravalent tetraspecific pan-sarbecovirus antibodies shown in FIG. 38 are shown in Table E14.

TABLE E14
Production of tetravalent tetraspecific anti-SARS-CoV-2 antibodies.

| | | | | | | Yield | | Yield |
|---|---|---|---|---|---|---|---|---|
| | Molecular | | | | | after | Yield after | after |
| | Weight | Isoelectric | Extinction | | | protein A | concentration | SEC |
| ID | (kDa) | Point | Coefficient | Cells | Scale (L) | (mg/L) | (mg/L) | (mg/L) |
| MX1870 | 202 | 7.91 | 1.521 | ExpiCHO | 0.2 | 15.68 | 9.52 | 2.16 |
| MX1871 | 202 | 7.91 | 1.521 | ExpiCHO | 0.2 | 11.01 | 4.67 | 1.60 |
| MX1872 | 202 | 7.89 | 1.521 | ExpiCHO | 0.2 | 17.61 | 6.30 | 2.93 |
| MX1873 | 202 | 7.89 | 1.521 | ExpiCHO | 0.2 | 16.96 | 9.56 | 3.84 |
| MX1874 | 202 | 7.91 | 1.521 | ExpiCHO | 0.2 | 12.74 | 5.68 | 1.90 |
| MX1875 | 202 | 7.91 | 1.521 | ExpiCHO | 0.2 | 13.45 | 5.13 | 2.26 |
| MX1876 | 202 | 7.9 | 1.521 | ExpiCHO | 0.2 | 23.27 | 10.01 | 4.12 |
| MX1877 | 202 | 7.9 | 1.521 | ExpiCHO | 0.2 | 20.75 | 12.82 | 2.55 |
| MX1878 | 202 | 7.91 | 1.521 | ExpiCHO | 0.2 | 18.19 | 6.84 | 3.14 |
| MX1879 | 202 | 7.91 | 1.521 | ExpiCHO | 0.2 | 14.75 | 7.84 | 2.95 |
| MX1880 | 202 | 7.9 | 1.521 | ExpiCHO | 0.2 | 19.59 | 6.04 | 3.63 |
| MX1881 | 202 | 7.9 | 1.521 | ExpiCHO | 0.2 | 17.04 | 7.50 | 3.42 |

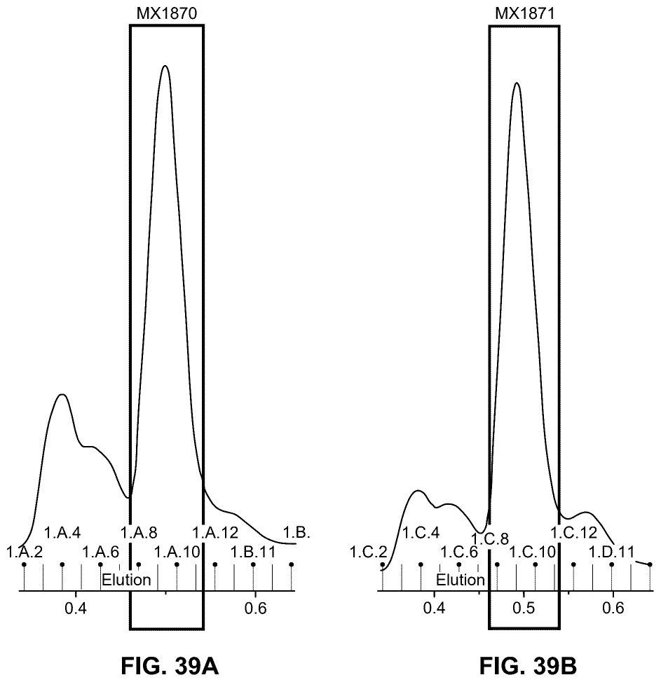

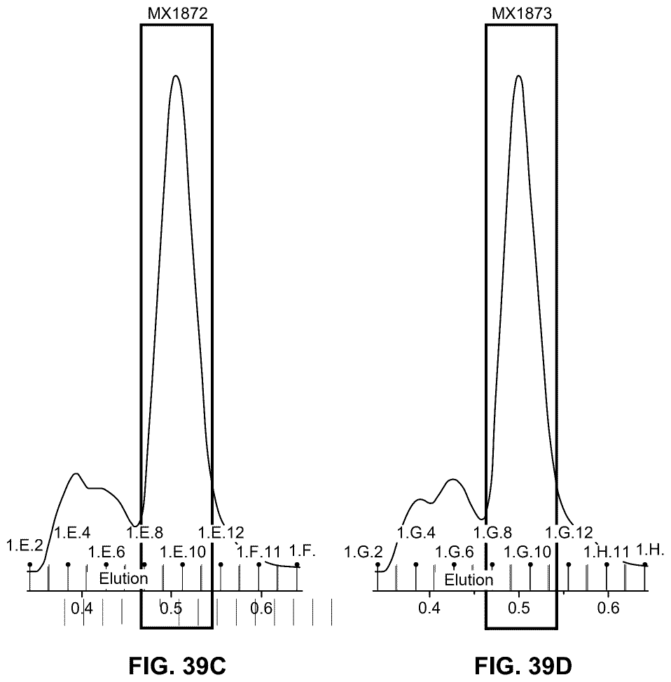

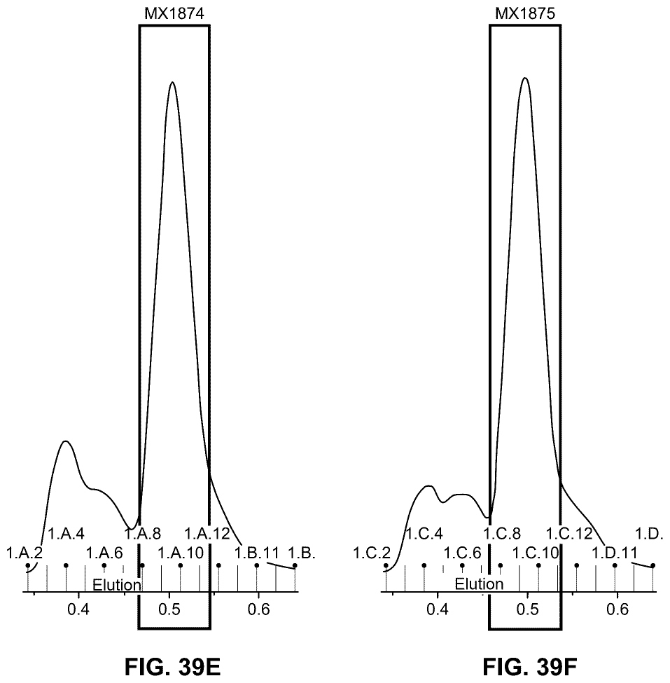

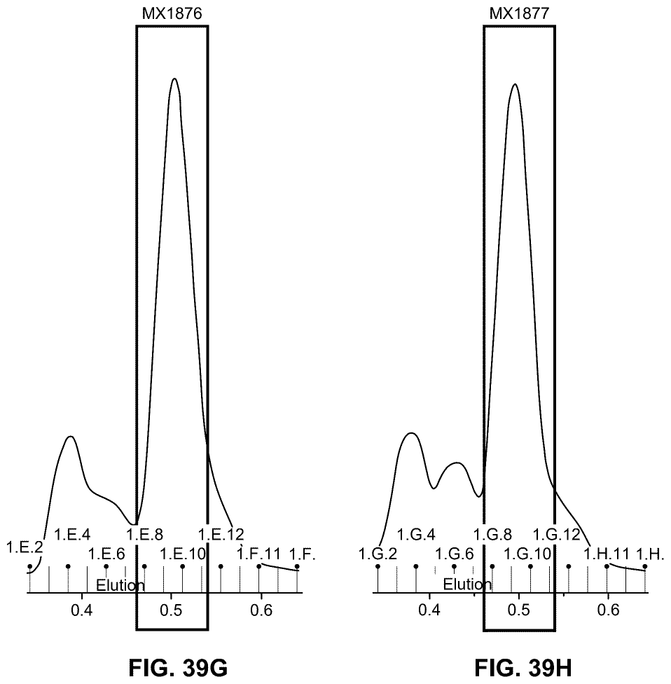

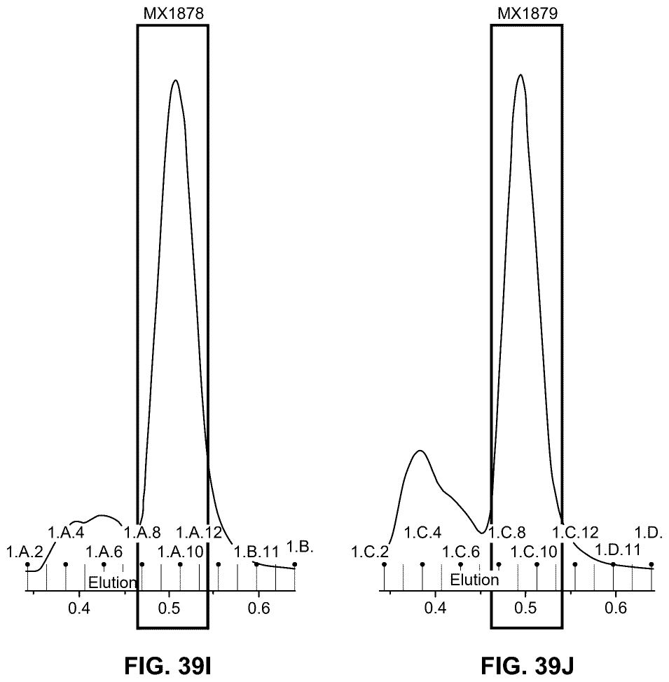

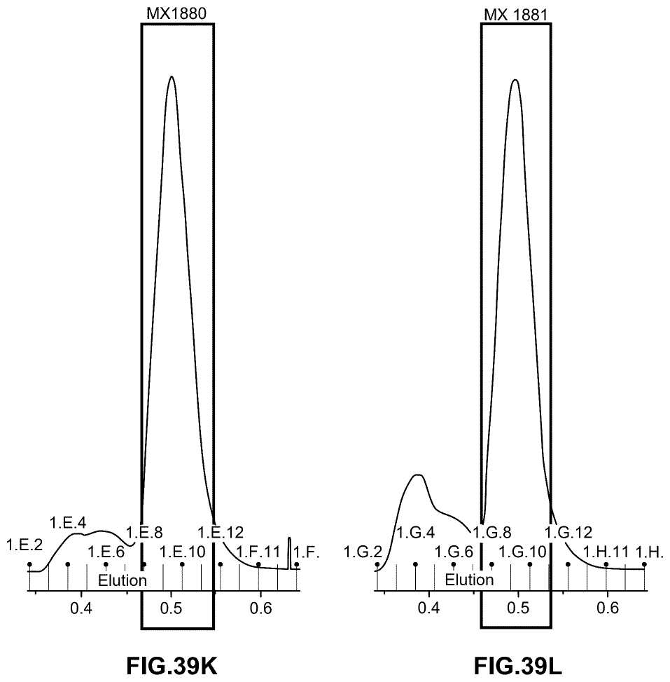

The SEC profiles of tetravalent tetraspecific anti-SARS-CoV-2 antibodies were obtained and are shown in FIGS. 39A-39L, SEC purification was performed with Superdex 200 Increase 10/300 GL (Cytiva). Fractions (shown in boxes in FIG. 39A-39L) were collected and pooled in 10 mM Histidine, pH 6.0/150 mM NaCl.

### Example 13

### Neutralizing Activity Against Recent SARS-CoV-2 Variants

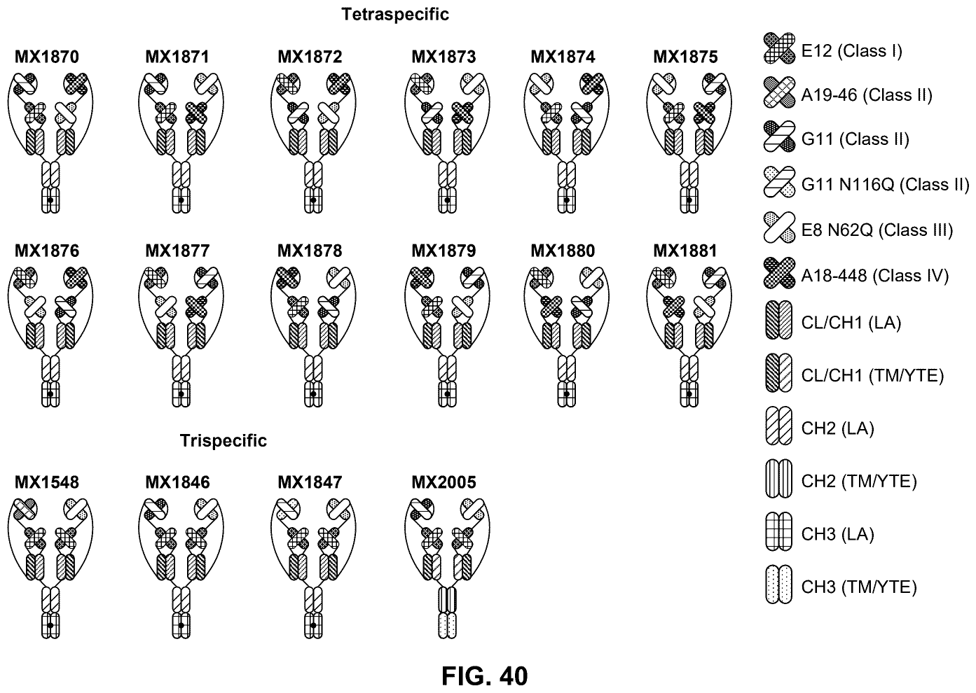

The neutralizing activity against recent SARS-CoV-2 variants was tested for the constructs shown in FIG. 40. The results of the analysis are shown in Table E15.

TABLE E15
Neutralizing activity against recent SARS-CoV-2 variants. IC50 and IC80 are expressed in nM.

| | | | | BA.2.86 | | EG.5.1 | | XBB.1.16.6 | |
|---|---|---|---|---|---|---|---|---|---|
| Specificity | Antibody | Class | CL/CH1 | IC50 | IC80 | IC50 | IC80 | IC50 | IC80 |
| Tetraspecific | MX1870 | I & II & III & IV | v1 | 0.1251 | 0.3859 | 0.0380 | 0.0957 | 0.0358 | 0.0909 |
| | MX1871 | I & II & III & IV | v1 | 0.0905 | 0.2475 | 0.0264 | 0.0561 | 0.0347 | 0.0772 |
| | MX1872 | I & II & III & IV | v1 | 0.5077 | 1.2613 | 0.0938 | 0.1884 | 0.0560 | 0.2144 |
| | MX1873 | I & II & III & IV | v1 | 0.1313 | 0.6043 | 0.0302 | 0.0579 | 0.0179 | 0.0476 |
| | MX1874 | I & II & III & IV | v1 | 0.1115 | 0.3572 | 0.0432 | 0.0832 | 0.0260 | 0.0750 |
| | MX1875 | I & II & III & IV | v1 | 0.1274 | 0.3890 | 0.0243 | 0.0680 | 0.0282 | 0.0845 |
| | MX1876 | I & II & III & IV | v1 | 0.6422 | 1.4514 | 0.0309 | 0.0959 | 0.0497 | 0.1221 |
| | MX1877 | I & II & III & IV | v1 | 0.5473 | 1.4453 | 0.0332 | 0.1079 | 0.0484 | 0.1852 |
| | MX1878 | I & II & III & IV | v1 | 0.2196 | 0.9204 | 0.0286 | 0.0687 | 0.0431 | 0.1044 |
| | MX1879 | I & II & III & IV | v1 | 0.3869 | 0.7861 | 0.0280 | 0.0690 | 0.0451 | 0.1015 |
| | MX1880 | I & II & III & IV | v1 | 0.2034 | 0.5733 | 0.0135 | 0.0332 | 0.0087 | 0.0341 |
| | MX1881 | I & II & III & IV | v1 | 0.2279 | 0.6670 | 0.0208 | 0.0624 | 0.0179 | 0.0489 |
| Trispecific | MX1548 | I & II & III | v1 | 0.1000 | 0.2408 | 0.3508 | 0.9208 | 0.2874 | 0.8466 |
| | MX1846 | I & II & III | v1 | 0.1557 | 0.5331 | 0.0477 | 0.1497 | 0.0472 | 0.1361 |
| | MX1847 | I & II & III | v1 | 0.1742 | 0.4701 | 0.0491 | 0.1354 | 0.0380 | 0.1681 |
| | MX2005 | I & II & III | v1 | N.T. | N.T. | N.T. | N.T. | N.T. | N.T. |
| Monoclonal | E12 | I | N/A | 0.0816 | 0.3672 | 1.3623 | 2.7333 | 2.3194 | 6.5314 |
| | G11 | II | N/A | >67 | >67 | 0.5393 | 1.6607 | 0.9549 | 2.4525 |
| | E8 | III | N/A | >67 | >67 | 0.6078 | 2.2409 | 0.5781 | 5.6570 |
| | B2 | III | N/A | 0.5931 | 1.4237 | 0.5074 | 1.4069 | 0.9102 | 2.4031 |
| | A18-448 | I/IV | N/A | 0.0355 | 0.1007 | 0.0215 | 0.0527 | 0.0232 | 0.0694 |
| | | | | FL.1.5.1 | | HV.1 | | HK.3 | |
| Specificity | Antibody | Class | CL/CH1 | IC50 | IC80 | IC50 | IC80 | IC50 | IC80 |
| Tetraspecific | MX1870 | I & II & III & IV | v1 | 0.0895 | 0.1784 | 0.0174 | 0.0506 | 0.0404 | 0.1329 |
| | MX1871 | I & II & III & IV | v1 | 0.0340 | 0.1065 | 0.0208 | 0.0674 | 0.0296 | 0.0885 |
| | MX1872 | I & II & III & IV | v1 | 0.1300 | 0.3327 | 0.0287 | 0.1150 | 0.0448 | 0.2028 |
| | MX1873 | I & II & III & IV | v1 | 0.0442 | 0.1405 | 0.0158 | 0.0451 | 0.0214 | 0.0683 |
| | MX1874 | I & II & III & IV | v1 | 0.0261 | 0.0719 | 0.0089 | 0.0289 | 0.0120 | 0.0565 |
| | MX1875 | I & II & III & IV | v1 | 0.0852 | 0.1400 | 0.0256 | 0.1124 | 0.0338 | 0.1021 |
| | MX1876 | I & II & III & IV | v1 | 0.0840 | 0.1748 | 0.0297 | 0.0928 | 0.0238 | 0.0867 |
| | MX1877 | I & II & III & IV | v1 | 0.1412 | 0.2908 | 0.0272 | 0.0979 | 0.0293 | 0.0872 |
| | MX1878 | I & II & III & IV | v1 | 0.0485 | 0.1093 | 0.0115 | 0.0566 | 0.0202 | 0.0810 |
| | MX1879 | I & II & III & IV | v1 | 0.0781 | 0.1573 | 0.0029 | 0.0394 | 0.0107 | 0.0628 |
| | MX1880 | I & II & III & IV | v1 | 0.0466 | 0.0993 | 0.0160 | 0.0464 | 0.0202 | 0.0606 |
| | MX1881 | I & II & III & IV | v1 | 0.0832 | 0.1765 | 0.0203 | 0.0608 | 0.0322 | 0.1091 |
| Trispecific | MX1548 | I & II & III | v1 | 0.6478 | 1.5586 | 0.3041 | 0.8764 | 0.2699 | 1.0756 |
| | MX1846 | I & II & III | v1 | 0.0873 | 0.2480 | 0.0451 | 0.1835 | 0.0291 | 0.1756 |
| | MX1847 | I & II & III | v1 | 0.0787 | 0.2470 | 0.0435 | 0.2312 | 0.0175 | 0.2677 |
| | MX2005 | I & II & III | v1 | N.T. | N.T. | N.T. | N.T. | N.T. | N.T. |
| Monoclonal | E12 | I | N/A | 1.2147 | 2.3973 | 1.7517 | 5.0378 | 3.1117 | 7.5053 |
| | G11 | II | N/A | 0.4259 | 1.7817 | 0.4669 | 1.9743 | 1.1365 | 2.1096 |
| | E8 | III | N/A | 0.7095 | 3.4473 | 0.5431 | 2.1784 | 1.3306 | 9.8044 |
| | B2 | III | N/A | 0.7179 | 1.9477 | 0.7734 | 1.8961 | 1.2756 | 2.4173 |
| | A18-448 | I/IV | N/A | 0.0196 | 0.0587 | 0.0199 | 0.0657 | 0.0194 | 0.0724 |
| | | | | JN.1 | | JD.1.1 | | SARS-CoV-1 | |
| Specificity | Antibody | Class | CL/CH1 | IC50 | IC80 | IC50 | IC80 | IC50 | IC80 |
| Tetraspecific | MX1870 | I & II & III & IV | v1 | 0.7357 | 2.2640 | 0.0776 | 0.2437 | 16.4100 | 30.0470 |
| | MX1871 | I & II & III & IV | v1 | 0.5872 | 2.1644 | 0.0248 | 0.0835 | 3.5560 | 9.5620 |
| | MX1872 | I & II & III & IV | v1 | 2.8202 | 9.1697 | 0.0956 | 0.3757 | 13.3450\| | 54.5850 |
| | MX1873 | I & II & III & IV | v1 | 0.6117 | 2.8244 | 0.0394 | 0.1371 | 4.9790 | 13.8530 |
| | MX1874 | I & II & III & IV | v1 | 0.1991 | 2.6788 | 0.0272 | 0.0802 | 17.3610 | 59.8230 |
| | MX1875 | I & II & III & IV | v1 | 1.3718 | 3.1806 | 0.0728 | 0.1847 | 4.3500 | 10.8360 |
| | MX1876 | I & II & III & IV | v1 | 4.9894 | 12.7500 | 0.0592 | 0.1769 | 24.8330 | >67 |
| | MX1877 | I & II & III & IV | v1 | 2.7166 | 5.4960 | 0.1135 | 0.3333 | 3.9560 | 13.3460 |
| | MX1878 | I & II & III & IV | v1 | 1.2255 | 3.2095 | 0.0156 | 0.0700 | 7.6690 | 15.2140 |
| | MX1879 | I & II & III & IV | v1 | 1.3982 | 2.3307 | 0.0456 | 0.1452 | 3.9230 | 21.9940 |
| | MX1880 | I & II & III & IV | v1 | 0.6469 | 1.6624 | 0.0346 | 0.1073 | 1.4030 | 5.3720 |
| | MX1881 | I & II & III & IV | v1 | 0.9819 | 2.6126 | 0.0829 | 0.2105 | 1.4980 | 3.0540 |
| Trispecific | MX1548 | I & II & III | v1 | N.T. | N.T. | N.T. | N.T. | >67 | >67 |
| | MX1846 | I & II & III | v1 | 6.3401 | 21.8400 | 0.1641 | 0.5747 | >67 | >67 |
| | MX1847 | I & II & III | v1 | 7.3137 | 26.7700 | 0.1858 | 0.6140 | >67 | >67 |
| | MX2005 | I & II & III | v1 | 3.8090 | 12.6100 | 0.2013 | 0.6057 | N.T. | N.T. |
| Monoclonal | E12 | I | N/A | 2.0514 | 5.5979 | 30.6700 | >67 | >67 | >67 |
| | G11 | II | N/A | >67 | >67 | 0.5067 | 1.7131 | >67 | >67 |
| | E8 | III | N/A | N.T. | N.T. | N.T. | N.T. | >67 | >67 |
| | B2 | III | N/A | 0.4655 | 0.8315 | 0.5041 | 1.1714 | 0.8382 | 2.2718 |
| | A18-448 | I/IV | N/A | 0.0367 | 0.1280 | 0.0194 | 0.0779 | 0.0328 | 0.1100 |

### Example 14

### Optimization of Well-Performing Antigen Binding Polypeptide Complexes-2

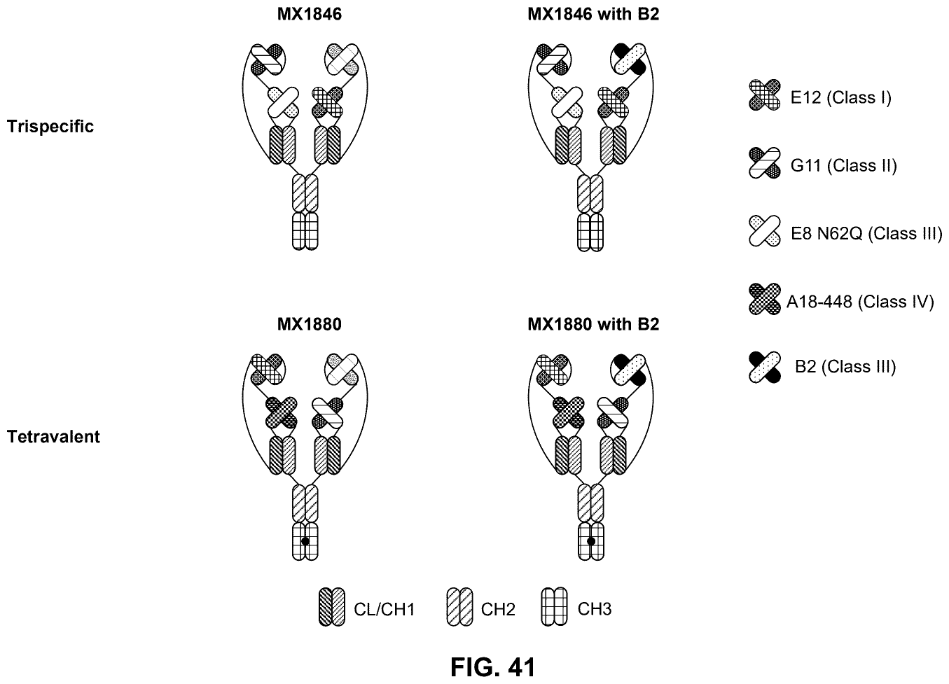

MX1846 is modified to include B2, obtaining MX1846 with B2 (FIG. 41). Similarly, MX1880 is also modified to include B2 to obtain MX1880 with B2 (FIG. 41).

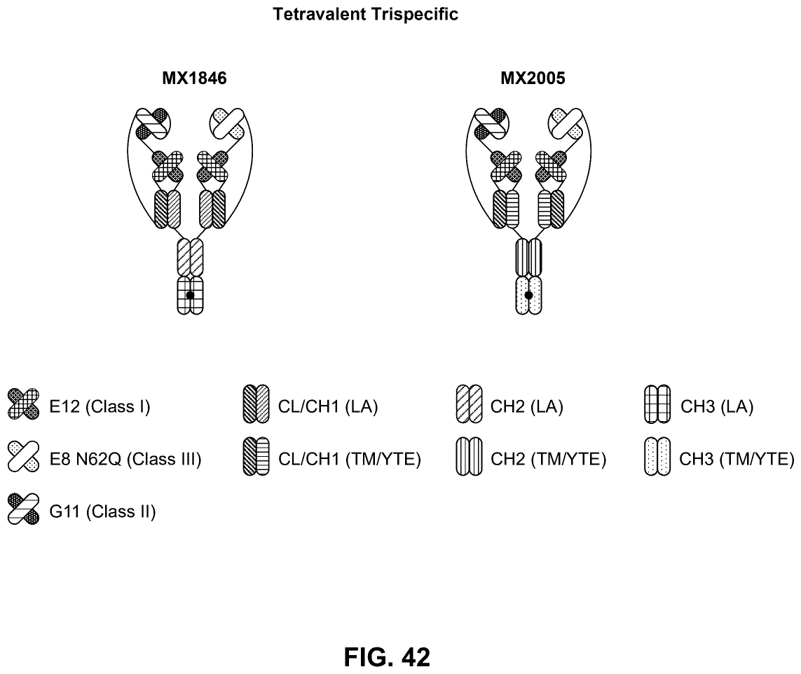

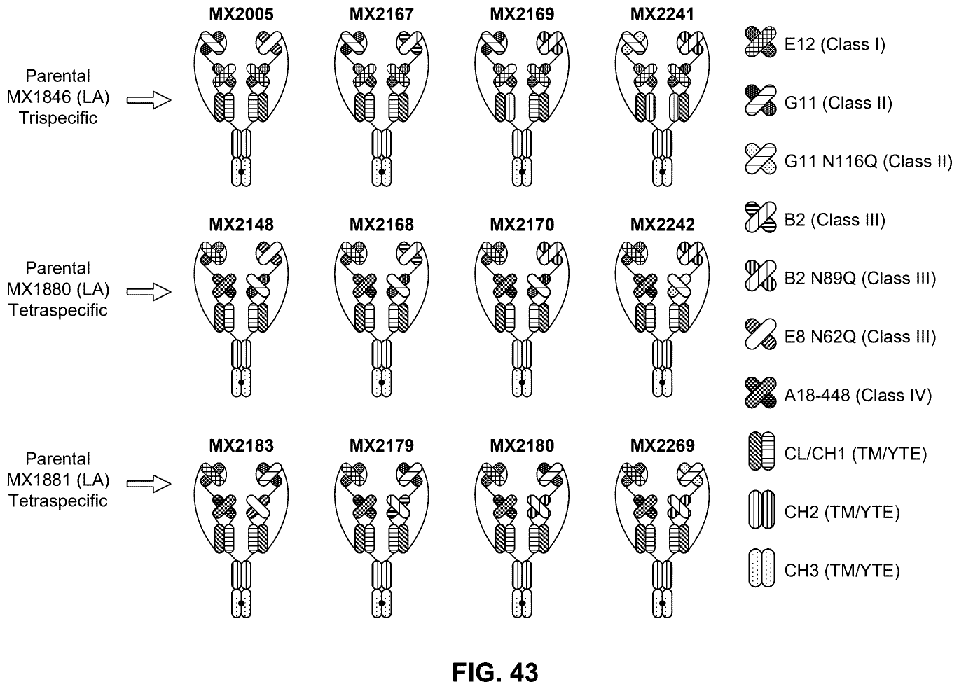

MX1846, comprising a LA (M428L/N434A) modification was modified to obtain MX2005 comprising a YTE (M252Y/S254T/T256E) and a TM (L234F/L235E/P331S) modification (FIG. 42 and FIG. 43). The VL and the VH of E8 in MX2005 were substituted with the VL and the VH of B2 as to obtain MX2167. Further, MX2167 was modified to introduce the N89Q modification into B2 as to obtain MX2169, MX2169 was further modified to introduce the N116Q modification into G11 as to obtain MX2241 (FIG. 43).

MX1880, comprising a LA (M428L/N434A) modification was modified to obtain MX2148 comprising a YTE (M252Y/S254T/T256E) and a TM (L234F/L235E/P331S) modification. The VL and the VH of E8 in MX2148 were substituted with the VL and the VH of B2 as to obtain MX2168. Further, MX2168 was modified to introduce the N89Q modification into B2 as to obtain MX2170, MX2170 was further modified to introduce the N116Q modification into G11 as to obtain MX2242 (FIG. 43).

MX1881, comprising a LA (M428L/N434A) modification was modified to obtain MX2183 comprising a YTE (M252Y/S254T/T256E) and a TM (L234F/L235E/P331S) modification. The VL and the VH of E8 in MX2183 were substituted with the VL and the VH of B2 as to obtain MX2179. Further, MX2179 was modified to introduce the N89Q modification into B2 as to obtain MX2180, MX2180 was further modified to introduce the N116Q modification into G11 as to obtain MX2269 (FIG. 43).

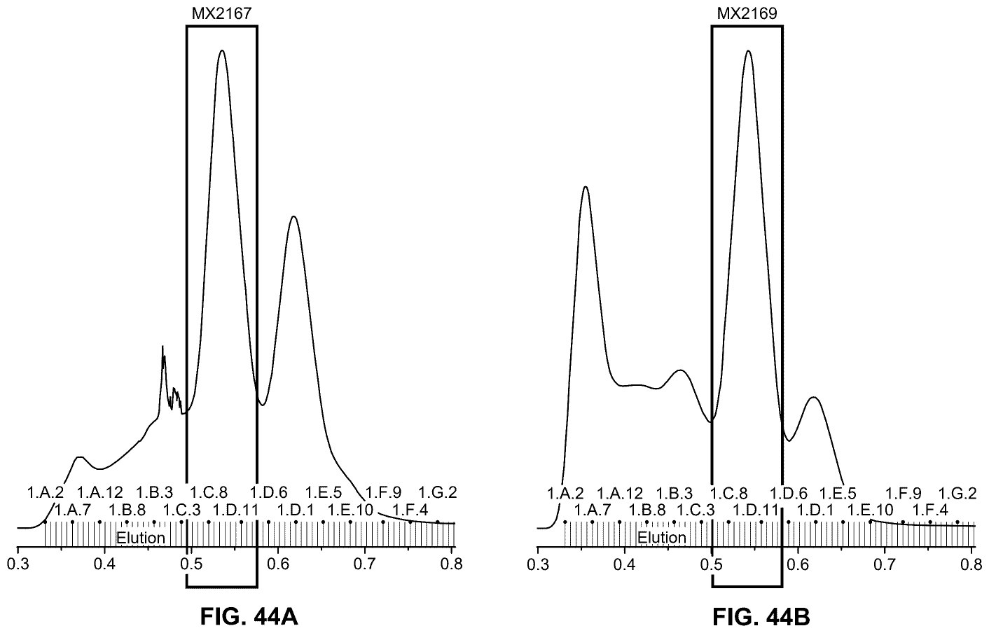

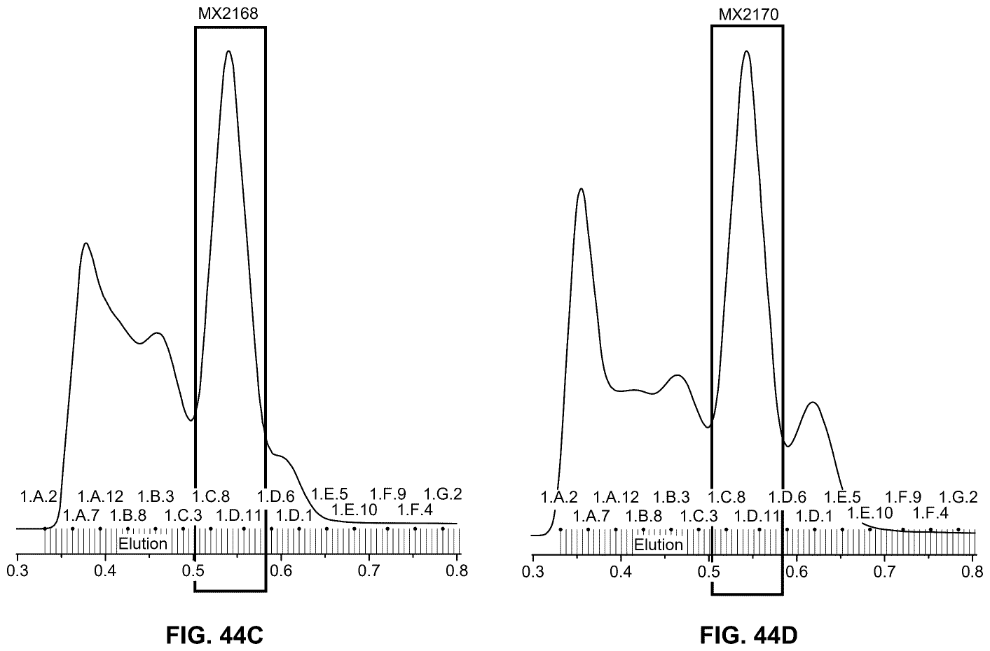

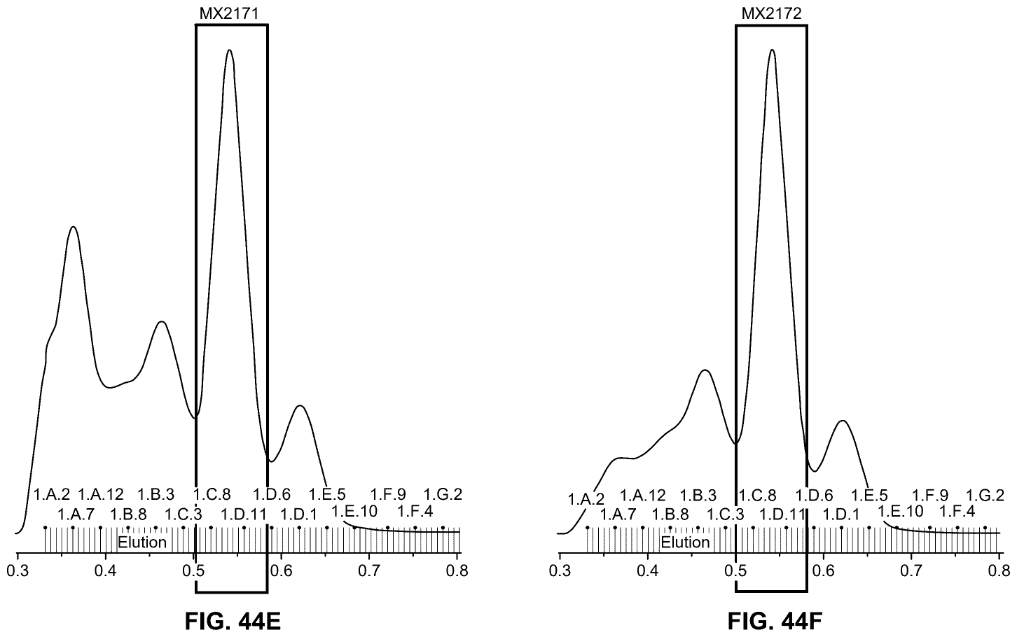

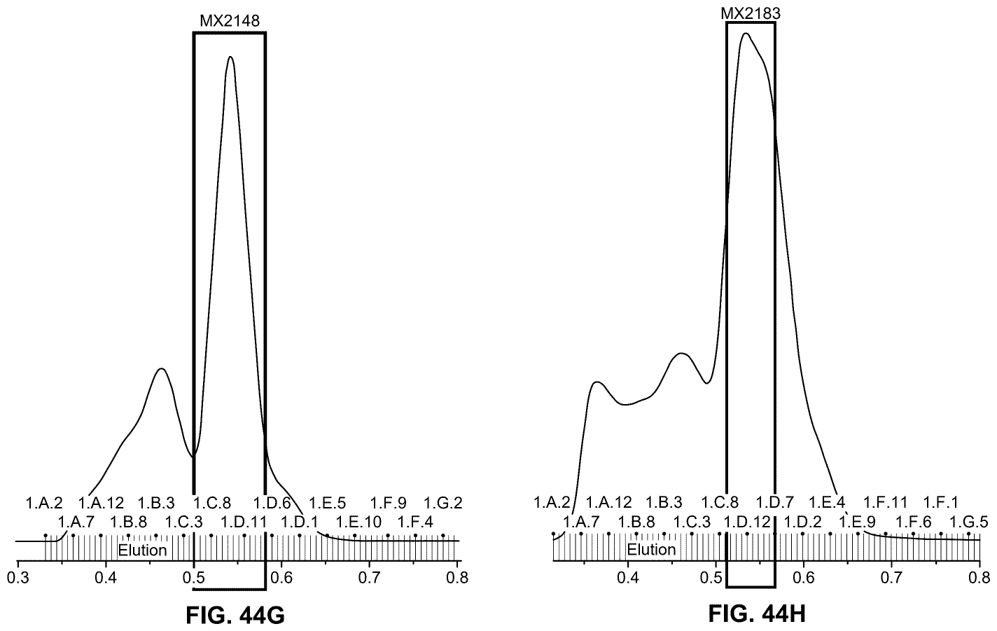

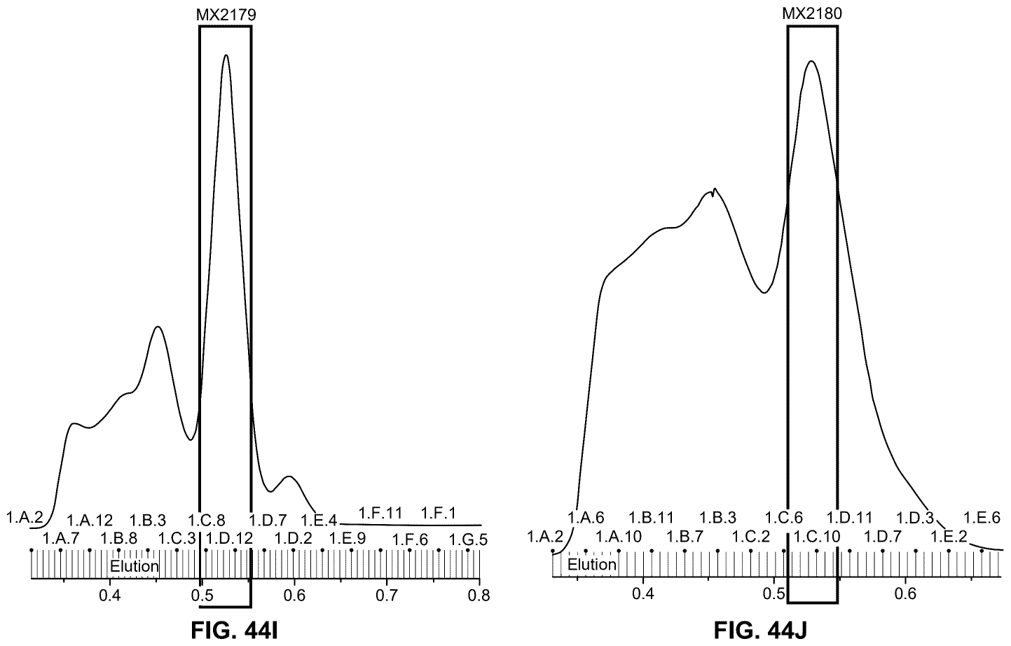

The full set of antigen polypeptide complexes tested in the experiments below are shown in FIG. 43, and comprise the shown modifications. The antigen polypeptide complexes shown in FIG. 43 were produced, and their SEC profiles are shown in FIGS. 44A-44J, SEC purification was performed with HiLoad 26/600 Superdex 200 pg (Cytiva), and fractions (shown in boxes in FIGS. 44A-44J) were collected and pooled in 10 mM Histidine, pH 6.0/150 mM NaCl.

The results of the analysis of the produced constructs is shown in Table E16.

TABLE E16
Production of tetravalent tetraspecific anti-SARS-CoV-2 antibodies.

| | | | | | | Yield | | Yield |
|---|---|---|---|---|---|---|---|---|
| | Molecular | | | | | after | Yield after | after |
| | Weight | Isoelectric | Extinction | | | protein A | concentration | SEC |
| ID | (kDa) | Point | Coefficient | Cells | Scale (L) | (mg/L) | (mg/L) | (mg/L) |
| MX2167 | 200 | 8.08 | 1.601 | CHO-K1 | 0.5 | 177.86 | 66.14 | 8.68 |
| MX2169 | 200 | 8.08 | 1.601 | CHO-K1 | 0.5 | 263.82 | 209.40 | 38.18 |
| MX2168 | 202 | 7.82 | 1.619 | CHO-K1 | 0.5 | 332.75 | 287.90 | 36.98 |
| MX2170 | 202 | 7.82 | 1.619 | CHO-K1 | 0.5 | 313.90 | 263.44 | 69.01 |
| MX2171 | 202 | 7.83 | 1.619 | CHO-K1 | 0.5 | 256.38 | 231.32 | 32.80 |
| MX2172 | 202 | 7.83 | 1.619 | CHO-K1 | 0.5 | 249.34 | 233.72 | 45.97 |
| MX2148 | 203 | 7.7 | 1.531 | CHO-K1 | 2 | 328.12 | 289.30 | 75.66 |
| MX2183 | 203 | 7.7 | 1.531 | CHO-K1 | 1 | 332.46 | 285.23 | 56.85 |
| MX2179 | 202 | 7.82 | 1.619 | CHO-K1 | 1 | 493.19 | 428.91 | 105.87 |
| MX2180 | 202 | 7.82 | 1.619 | CHO-K1 | 1 | 554.20 | 152.73 | 25.73 |

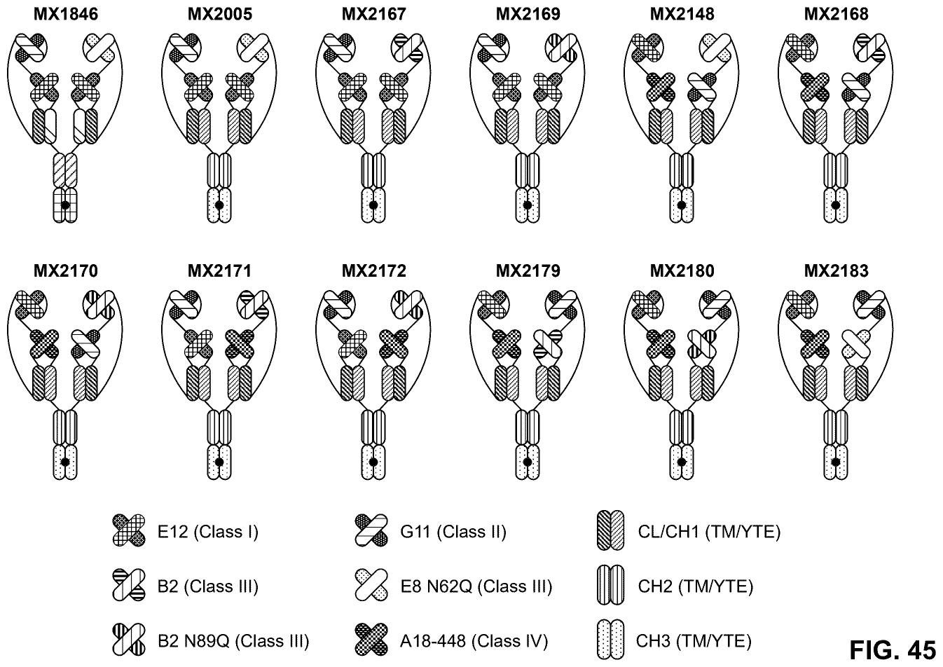

The neutralizing activities against 2 SARS-CoV-2 variants and SARS-CoV-1 were tested and are shown in Table E17. The tested antigen binding polypeptide complexes are shown in FIG. 45.

TABLE E17
Neutralizing activities against 2 SARS-CoV-2 variants and SARS-CoV-1. IC50 and IC80 are
expressed in nM.

| | | | HV.1 | | JN.1 | | SARS-CoV-1 | |
|---|---|---|---|---|---|---|---|---|
| Specificity | Antibody | RBD Class | IC50 | IC80 | IC50 | IC80 | IC50 | IC80 |
| Trispecific | MX1846 | I & II & III | 0.1306 | 0.4383 | 0.4593 | 2.5062 | N.T. | N.T. |
| | MX2005 | I & II & III | 0.134 | 0.5472 | 1.0912 | 11.9348 | >67 | >67 |
| | MX2167 | I & II & III | 0.4803 | 1.4334 | 0.6616 | 2.9355 | >67 | >67 |
| | MX2169 | I & II & III | 0.2068 | 1.2394 | 0.3848 | 4.8004 | >67 | >67 |
| Tetraspecific | MX2148 | I & II & III | 0.0291 | 0.1488 | 0.0734 | 0.3806 | 0.38 | 0.9232 |
| | | & IV | | | | | | |
| | MX2168 | I & II & III | 0.1103 | 0.3435 | 0.1032 | 0.4798 | 0.1373 | 0.6332 |
| | | & IV | | | | | | |
| | MX2170 | I & II & III | 0.1085 | 0.3416 | 0.2164 | 0.6219 | 0.1297 | 0.5432 |
| | | & IV | | | | | | |
| | MX2171 | I & II & III | 0.0833 | 0.432 | 0.079 | 0.4515 | 1.2056 | 3.9699 |
| | | & IV | | | | | | |
| | MX2172 | I & II & III | 0.0552 | 0.317 | 0.0495 | 0.4652 | 1.0844 | 2.5084 |
| | | & IV | | | | | | |
| | MX2179 | I & II & III | 0.0917 | 0.3102 | 0.0378 | 0.1773 | 0.1394 | 0.3804 |
| | | & IV | | | | | | |
| | MX2180 | I & II & III | 0.1000 | 0.3619 | 0.0352 | 0.1653 | 0.1905 | 0.4166 |
| | | & IV | | | | | | |
| | MX2183 | I & II & III | 0.1023 | 0.3522 | 0.0785 | 0.3908 | 0.3872 | 0.8191 |
| | | & IV | | | | | | |

MX2148 is a tetraspecific antigen binding complex comprising, the VL and the VH of: E12 (class I), A18-448 (class I/IV), E8 (class III), and G11 (class II), wherein E8 comprises a N62Q modification. Further, MX2148 comprises a CL/CH1 region and an Fc region (CH2-CH3) comprising a YTE (M252Y/S254T/T256E) and a TM (L234F/L235E/P331S) modification, MX2148 displayed t neutralization activity against all prior and current SARS-CoV-2 variants, including the dominant JN.1 strain as shown in the table below. The data shown herein suggest that the insertion of the VL and of the VH of A18-448 (class I/IV) into the antigen binding complex led to improved neutralization activity. Further analysis showed that A18-448 (class I/IV) is highly cross-reactive and capable of binding to both SARS-CoV-1 and SARS-CoV-2 variants.

A18-448 appears to bind to the RBD-up, to an epitope located in the same region to which Class IV antibodies can bind. Nevertheless, A18-448 appears to be capable of competing with Class I and Class III antibodies, as well as with ACE2. Based on the above, A18-448 can be considered as belonging to more than one of the anti-SARS-COV antibody classes as defined in the published literature. In some aspects, A18-448 can be considered a Class I/IV antibody as described in the paragraph above.

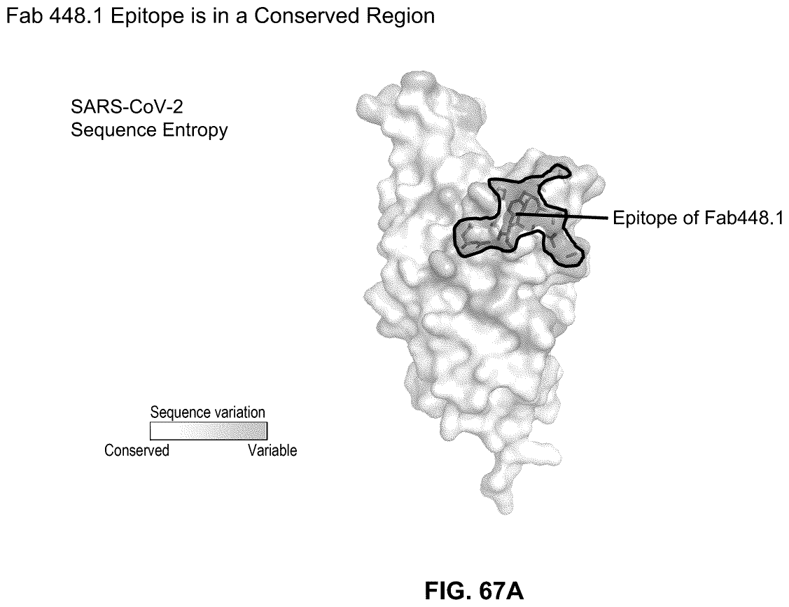

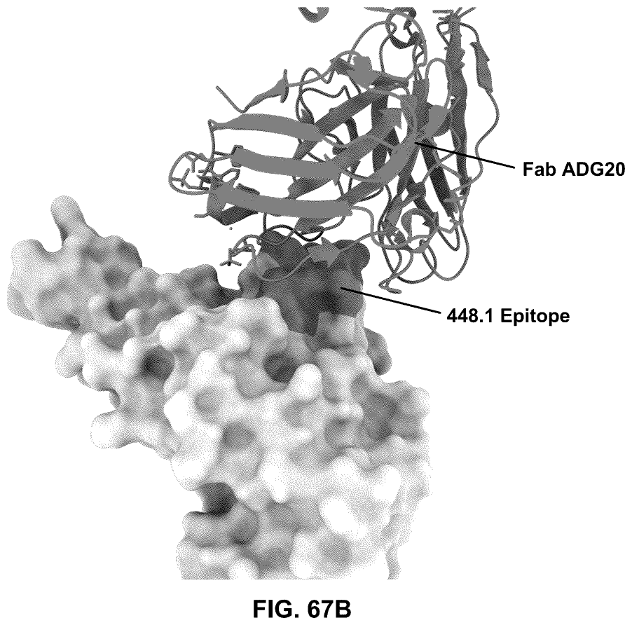

The epitope to which A18-448 binds (FIGS. 67A-67B) appeared to have a degree of conservation among different SARS-CoV-1 and SARS-CoV-2 variants, and amino acid variations in or near this epitope do not appear to affect the binding of A18-448 to the epitope, nor the potency of A18-448. The identified epitope comprises a contiguous loop of amino acids comprising residues 499-505 of SARS-COV and a short region of alpha helix comprising residues 404-406 of SARS-COV. These amino acid residues appeared to be quite conserved across SARS-COV variants. Additionally, A18-448 seemed to be capable of maintaining the binding capability to this epitope, despite the presence of amino acid variations, by binding to the main C-chain and not to the side chain of key residues comprised in the epitope.

The neutralizing activities of MX2148 against 28 SARS-CoV-2 variants are shown in Tables E18A and E18B.

Additionally, the tetravalent tetraspecific antigen binding complexes shown in FIGS. 38, 43, 45, 47, and 52 showed an anti-pan-sarbecovirus neutralizing activity.

TABLE E18A
Neutralizing activities of MX2148 against 28 SARS-CoV-2 variants. IC50 and IC80 are
expressed in nM.

| | | | WA1 | | D614G | | B.1.1.7 | | B.1.351 | |
|---|---|---|---|---|---|---|---|---|---|---|
| Specificity | Antibody | Class (RBD) | IC50 | IC80 | IC50 | IC80 | IC50 | IC80 | IC50 | IC80 |
| Tetraspecific | MX1880 | I & II & III | 0.3258 | 0.7995 | 0.1164 | 0.2356 | 0.1836 | 0.3956 | 0.1043 | 0.2393 |
| | (LA) | & IV | | | | | | | | |
| | MX2148 | I & II & III | 0.2601 | 0.7308 | 0.1132 | 0.2887 | 0.1299 | 0.3461 | 0.0934 | 0.2649 |
| | (TM/YTE) | & IV | | | | | | | | |
| Monoclonal | AZD3152 | I | 1.0767 | 2.2013 | 0.1022 | 0.6227 | 0.6216 | 1.5406 | 0.1633 | 0.4586 |
| | | | P.1 | | B.1.617.2 | | B.1.427 | | B.1.429 | |
| Specificity | Antibody | Class (RBD) | IC50 | IC80 | IC50 | IC80 | IC50 | IC80 | IC50 | IC80 |
| Tetraspecific | MX1880 | I & II & III | 0.0882 | 0.2721 | 0.4002 | 0.4523 | 0.3064 | 0.4772 | 0.1060 | 0.2243 |
| | (LA) | & IV | | | | | | | | |
| | MX2148 | I & II & III | 0.0559 | 0.2688 | 0.2397 | 0.6976 | 0.2329 | 0.6079 | 0.0724 | 0.2483 |
| | (TM/YTE) | & IV | | | | | | | | |
| Monoclonal | AZD3152 | I | 0.3151 | 0.5480 | 0.6015 | 2.2014 | 0.5982 | 2.0321 | 0.5494 | 1.3875 |
| | | | B.1.525 | | B.1.526 | | B.1.617.1 | | BA.1 | |
| Specificity | Antibody | Class (RBD) | IC50 | IC80 | IC50 | IC80 | IC50 | IC80 | IC50 | IC80 |
| Tetraspecific | MX1880 | I & II & III | 0.2118 | 0.3484 | 0.0900 | 0.1837 | 0.0878 | 0.1700 | 0.0373 | 0.1006 |
| | (LA) | & IV | | | | | | | | |
| | MX2148 | I & II & III | 0.2256 | 0.3635 | 0.0785 | 0.1625 | 0.0520 | 0.1398 | 0.0253 | 0.0744 |
| | (TM/YTE) | & IV | | | | | | | | |
| Monoclonal | AZD3152 | I | 0.6680 | 1.5300 | 0.3293 | 1.0399 | 0.2706 | 0.6866 | 0.0779 | 0.4457 |
| | | | BA.4/5 | | P.2 | | B.1.621 | | CH.1.1 | |
| Specificity | Antibody | Class (RBD) | IC50 | IC80 | IC50 | IC80 | IC50 | IC80 | IC50 | IC80 |
| Tetraspecific | MX1880 | I & II & III | 0.0402 | 0.1533 | 0.1576 | 0.2775 | 0.1642 | 0.3657 | 0.0946 | 0.3037 |
| | (LA) | & IV | | | | | | | | |
| | MX2148 | I & II & III | 0.0400 | 0.1613 | 0.0919 | 0.2688 | 0.0984 | 0.1746 | 0.0775 | 0.2703 |
| | (TM/YTE) | & IV | | | | | | | | |
| Monoclonal | AZD3152 | I | 0.1975 | 0.6565 | 0.1353 | 0.3592 | 0.2646 | 0.6246 | 0.3014 | 1.5935 |
| | | | XBB | | XBB.1.5 | | XBB.2.3.2 | | BA.2.86 | |
| Specificity | Antibody | Class (RBD) | IC50 | IC80 | IC50 | IC80 | IC50 | IC80 | IC50 | IC80 |
| Tetraspecific | MX1880 | I & II & III | 0.0607 | 0.1709 | 0.0831 | 0.2239 | 0.0702 | 0.2609 | 0.0742 | 0.3426 |
| | (LA) | & IV | | | | | | | | |
| | MX2148 | I & II & III | 0.0422 | 0.1539 | 0.0604 | 0.1872 | 0.0831 | 0.3555 | 0.0325 | 0.1962 |
| | (TM/YTE) | & IV | | | | | | | | |
| Monoclonal | AZD3152 | I | 0.0748 | 0.3487 | 0.1089 | 0.3554 | 0.1677 | 0.5133 | 0.3515 | 2.2279 |
| | | | EG.5.1 | | FL.1.5.1 | | XBB.1.16.6 | | HV.1 | |
| Specificity | Antibody | Class (RBD) | IC50 | IC80 | IC50 | IC80 | IC50 | IC80 | IC50 | IC80 |
| Tetraspecific | MX1880 | I & II & III | 0.0683 | 0.2227 | 0.0966 | 0.2616 | 0.0638 | 0.1954 | 0.0483 | 0.1613 |
| | (LA) | & IV | | | | | | | | |
| | MX2148 | I & II & III | 0.0405 | 0.1450 | 0.0682 | 0.2181 | 0.0443 | 0.1377 | 0.0291 | 0.1488 |
| | TM/YTE) | & IV | | | | | | | | |
| Monoclonal | AZD3152 | I | >67 | >67 | >67 | >67 | >67 | >67 | >67 | >67 |
| | | | HK.3 | | JD.1.1 | | JF.1 | | JN.1 | |
| Specificity | Antibody | Class (RBD) | IC50 | IC80 | IC50 | IC80 | IC50 | IC80 | IC50 | IC80 |
| Tetraspecific | MX1880 | I & II & III | 0.0655 | 0.2276 | 0.0829 | 0.2478 | 0.0557 | 0.8556 | 0.1437 | 0.4884 |
| | (LA) | & IV | | | | | | | | |
| | MX2148 | I & II & III | 0.0295 | 0.1776 | 0.0477 | 0.1973 | 0.0674 | 0.1497 | 0.0734 | 0.3806 |
| | (TM/YTE) | & IV | | | | | | | | |
| Monoclonal | AZD3152 | I | >67 | >67 | >67 | >67 | >67 | >67 | 5.1079 | 27.9546 |

TABLE E18B
Neutralizing activities of MX2148 against 28 SARS-CoV-2 variants. IC50 and IC80 are expressed in
ng/ml.

| Specificity | Antibody | Class (RBD) | WA1 | D614G | B.1.1.7 | B.1.351 | P.1 | B.1.617.2 | B.1.427 |
|---|---|---|---|---|---|---|---|---|---|
| Tetraspecific | MX2148 | I & II & | 52 | 23 | 26 | 19 | 11 | 48 | 47 |
| | | III & IV | | | | | | | |
| Monoclonal | AZD3152* | I | 161 | 15 | 93 | 24 | 47 | 90 | 90 |
| Specificity | Antibody | Class (RBD) | B.1.429 | B.1.525 | B.1.526 | B.1.617.1 | BA.1 | BA.4/5 | P.2 |
| Tetraspecific | MX2148 | I & II & | 14 | 45 | 16 | 10 | 5 | 8 | 18 |
| | | III & IV | | | | | | | |
| | AZD3152* | I | 82 | 100 | 49 | 41 | 12 | 30 | 20 |
| Specificity | Antibody | Class (RBD) | B.1.621 | CH.1.1 | XBB | XBB.1.5 | XBB.2.3.2 | BA.2.86 | EG.5.1 |
| Tetraspecific | MX2148 | I & II & | 20 | 16 | 8 | 12 | 17 | 6 | 8 |
| | | III & IV | | | | | | | |
| Monoclonal | AZD3152 | I | 40 | 45 | 11 | 16 | 25 | 53 | >10,000 |
| Specificity | Antibody | Class (RBD) | FL.1.5.1 | XBB.1.16.6 | HV.1 | HK.3 | JD.1.1 | JF.1 | JN.1 |
| Tetraspecific | MX2148 | I & II & | 14 | 9 | 6 | 6 | 10 | 13 | 15 |
| | | III & IV | | | | | | | |
| Monoclonal | AZD3152 | I | >10,000 | >10,000 | >10,000 | >10,000 | >10,000 | >10,000 | 766 |

### Example 15

### Challenge in Hamsters

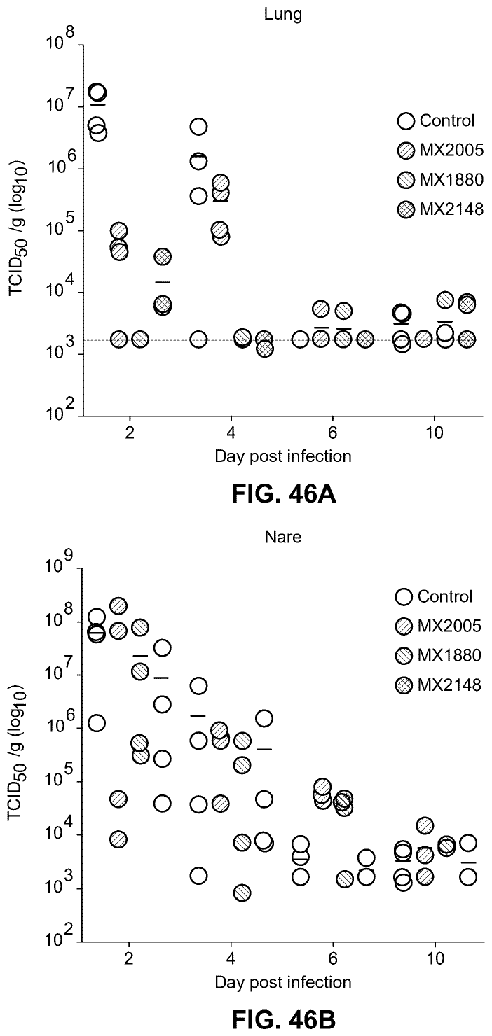

MX2005, MX1880 and MX2148 were tested in a hamster challenge experiment, performed as described in Example 6. The virus used in this hamster challenge was a SARS-Cov-2 omicron variant/JN.1 sub variant at a titer of 2.2×10⁶ TCID50/ml. The IN challenge was 4.4×10⁴ TCID50/dose and the antigen binding polypeptide complex was administered at a dose of 10 mg/kg via intraperitoneal administration route, MX2005, MX1880 and MX2148 showed protective efficacy against JN.1 variant (FIGS. 46A-46B).

### Example 16

Tetravalent Trispecific Anti-SARS-CoV-2 Antibodies with Fc Modifications

Different Fc modifications were tested to further extend antigen binding polypeptide complex half-life, PK data in humans indicated that AZD7442 (Evusheld) with triple mutations (TM) and YTE mutations has an extended half-life of ˜90 days and confers protection for 6 months.

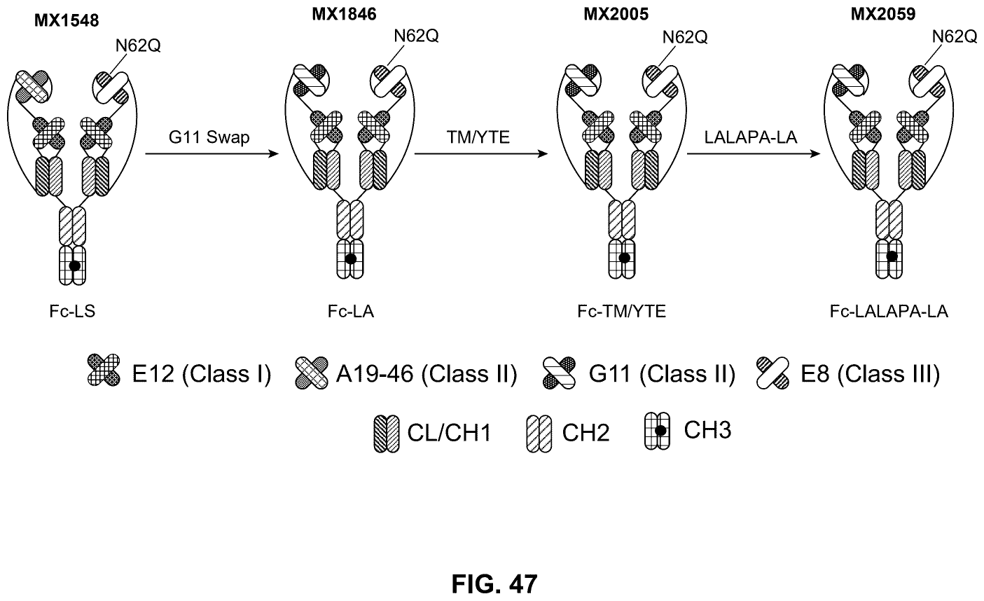

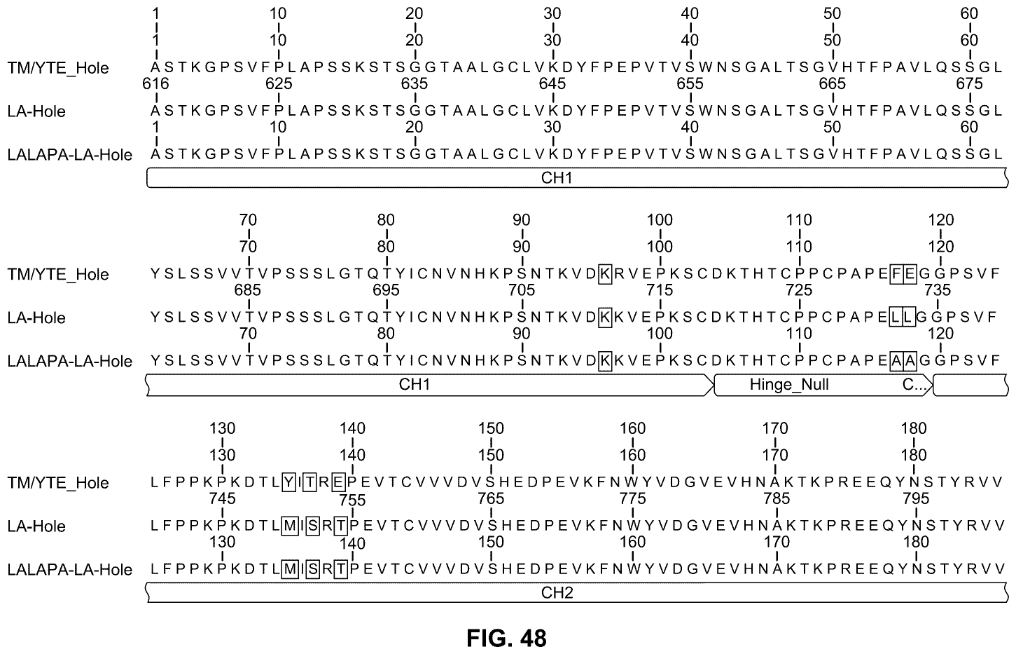

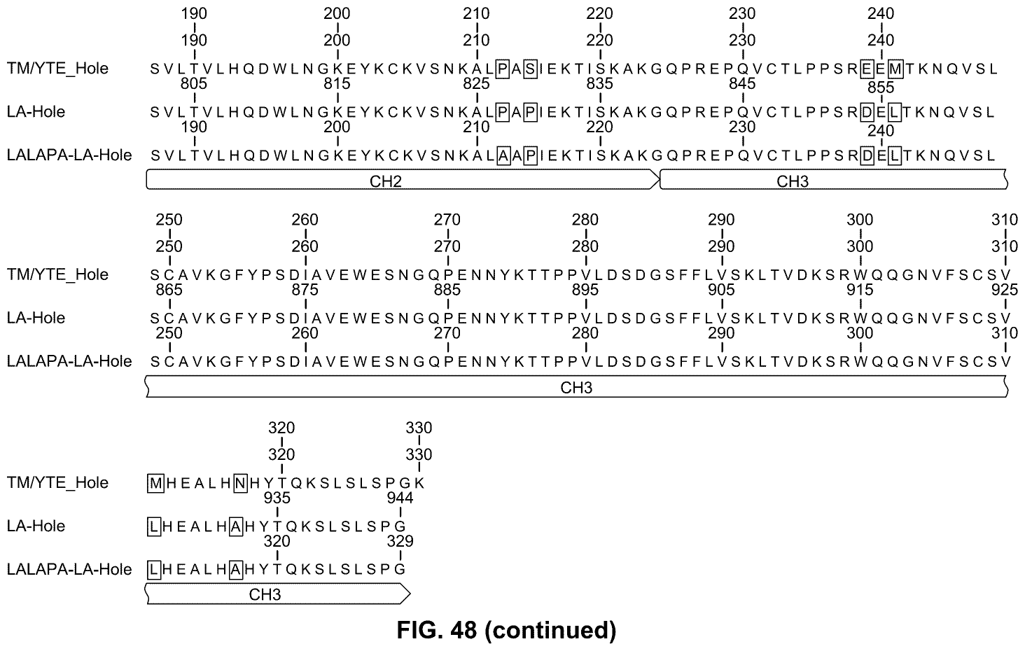

As described above, the VL and the VH of A19-46 in MX1548 were substituted with the VL and the VH of G11 as to obtain MX1846, which contains a LA modification. Two constructs based on MX1846 were produced to replace Fc-LA with Fc-TM/YTE (MX 2005) or Fc-LALAPA-LA (MX2059). A TM/YTE modification was inserted into MX1846 to obtain MX2005. Further, a LALAPA-LA modification was inserted into MX2055 to obtain MX2059, as shown in FIG. 47. The sequence alignments between Fc domains of TM/YTE, LALAPA-LA and LA are shown in FIG. 48 (with a hole modification as an example). The modification sites are as follows:
YTE half-life extension—Half-life extension
M252Y/S254T/T256E
Triple mutation (TM)—Attenuate Fcγ binding
L234F/L235E/P331S
Allotype (IGHG1.03)
K214R/D356E/L358M
LA—Half-life extension
M428L/N434A
LS—Half-life extension:
M428L/N433S. (can be present in both Knob-LS and Hole-LS).
LALAPA—Attenuate Fcg binding
L234A/L235A/P329A
Knob
S354C/T366W
Hole
Y349C/T366S/L368A/Y407V
With or without C-terminal Lys

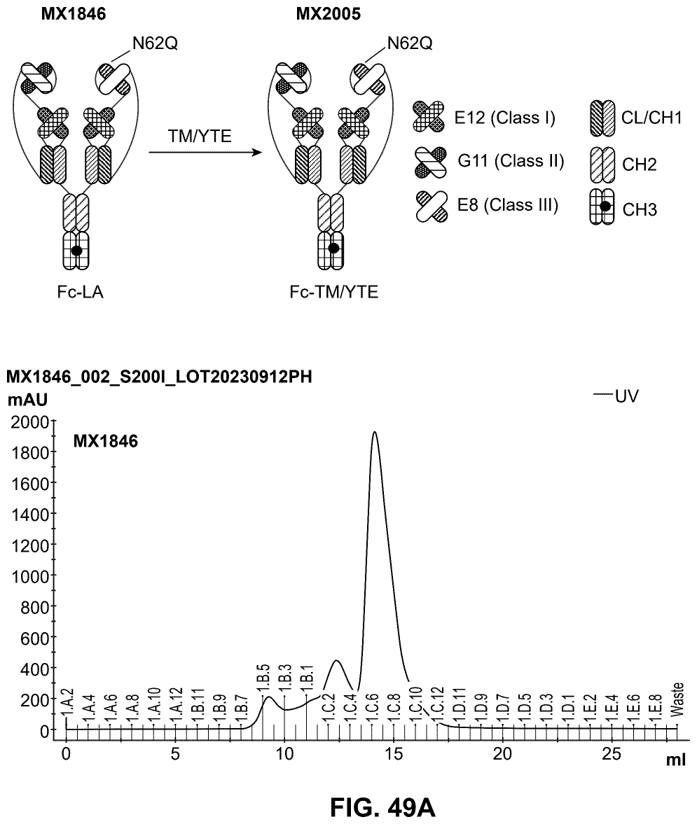

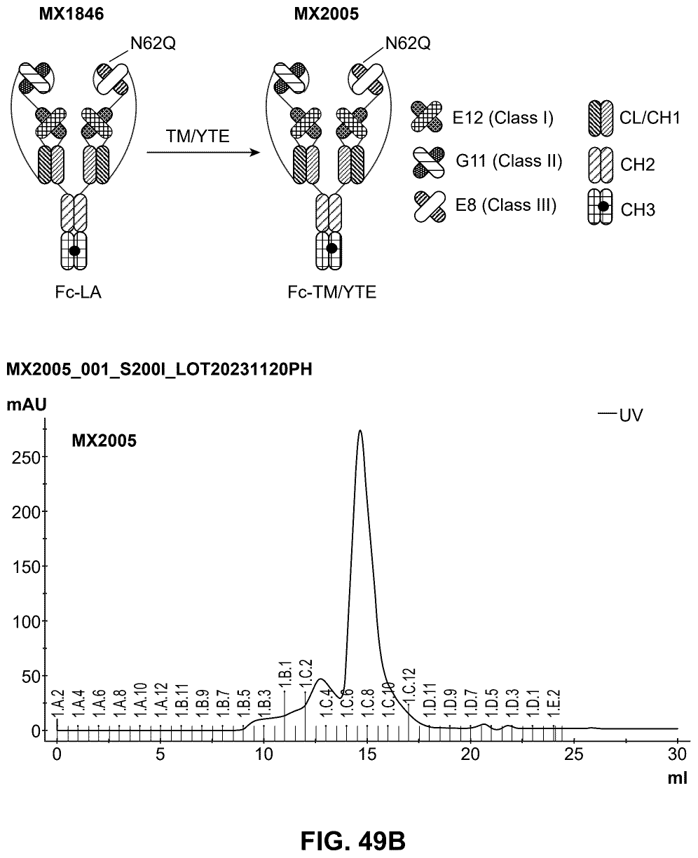

Size exclusion chromatography (SEC), using superdex 200 increase column, and 10 mM Histidine pH 6.0 and 150 mM NaCl as running buffer, was performed on MX1846 and MX2005. The results are shown in FIGS. 49A-49B.

MX1846 and MX2005 were expressed in ExpiCHO-S culture and titer measured by Octet BLI using protein A biosensor. The results are shown in Table E20.

TABLE E20
MX1846 and MX2005 expression.

| | | Day3 | | | Day7 | | | Day9 | | |
|---|---|---|---|---|---|---|---|---|---|---|
| | | Cell | | Titers | Cell | | Titers | Cell | | Titers |
| Scale (ml) | Name | conc | Viability | (mg/L) | conc | Viability | (mg/L) | conc | Viability | (mg/L) |
| 500 ml × 2 | MX1846 | | | | 9M | 89% | 73.1 | 10M | 72% | 72.2 |
| 400 ml × 1 | MX1846 | | | | | | | 9.7M | 78% | 97.6 |
| 10 ml × 1 | MX1846 | 12M | 98% | 26.5 | 7M | 86% | 61.9 | | | |
| 10 ml × 1 | MX1846 | 12M | 97% | 26.7 | | | | | | |
| 200 ml × 1 | MX2005 | 9.8M | 98% | 31.1 | | | | | | |
| 2.2 L X 1 | MX2005 | | | | | | | 11M | 65% | 81 |
| 10 ml × 1 | MX2005 | 9.8M | 98% | 28.3 | 12M | 93% | 69.9 | | | |
| 10 ml × 1 | MX2005 | 9.5M | 95% | 24.3 | | | | | | |

Titers and SEC profiles were comparable between MX1846 and MX2005.

The neutralizing activity of MX1846 and MX2005 was also measured, and the results are shown in Table E21.

TABLE E21
Neutralizing activities comparison between MX1846 and MX2005. IC50 and IC80 are
expressed in nM.

| | | | WA.1 | | Delta | | BA.2 | | BA.4/5 | | | |
|---|---|---|---|---|---|---|---|---|---|---|---|---|
| Specificity | Antibody | Fc | IC50 | IC80 | IC50 | IC80 | IC50 | IC80 | IC50 | | IC80 | |
| Trispecific | MX1846 | LA | 0.2513 | 0.4876 | 0.3129 | 0.4831 | 0.0522 | 0.0876 | 0.0429 | | 0.0949 | |
| Trispecific | MX2005 | TM/YTE | 0.1779 | 0.4439 | 0.2577 | 0.3010 | 0.0585 | 0.1012 | 0.0579 | | 0.1010 | |
| | | | BQ.1.1 | | BA.2.75.2 | | CH.1.1 | | BF.7 | | EG.5 | |
| Specificity | Antibody | Fc | IC50 | IC80 | IC50 | IC80 | IC50 | IC80 | IC50 | IC80 | IC50 | IC80 |
| Trispecific | MX1846 | LA | 0.0634 | 0.1955 | 0.0155 | 0.0539 | 0.0776 | 0.2392 | 0.0255 | 0.0740 | 0.0965 | 0.2017 |
| Trispecific | MX2005 | TM/YTE | 0.0558 | 0.1204 | 0.0345 | 0.0874 | 0.1705 | 0.4366 | 0.0372 | 0.0982 | 0.0271 | 0.0956 |

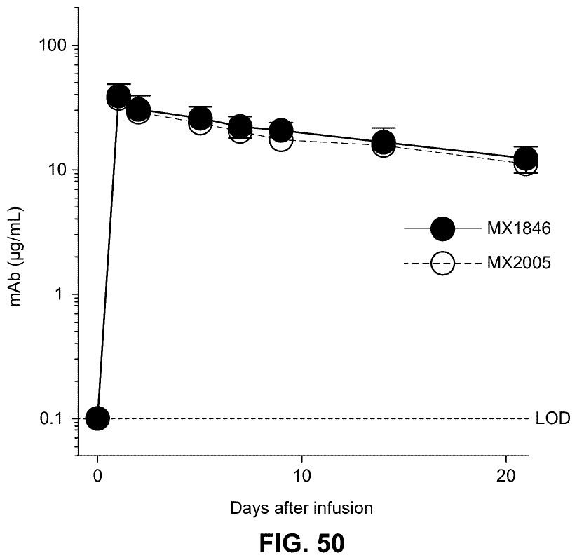

The activity of MX1846 and MX2005 was tested in FcRn-Fc mice as described in Example 9. The results are shown in FIG. 50.

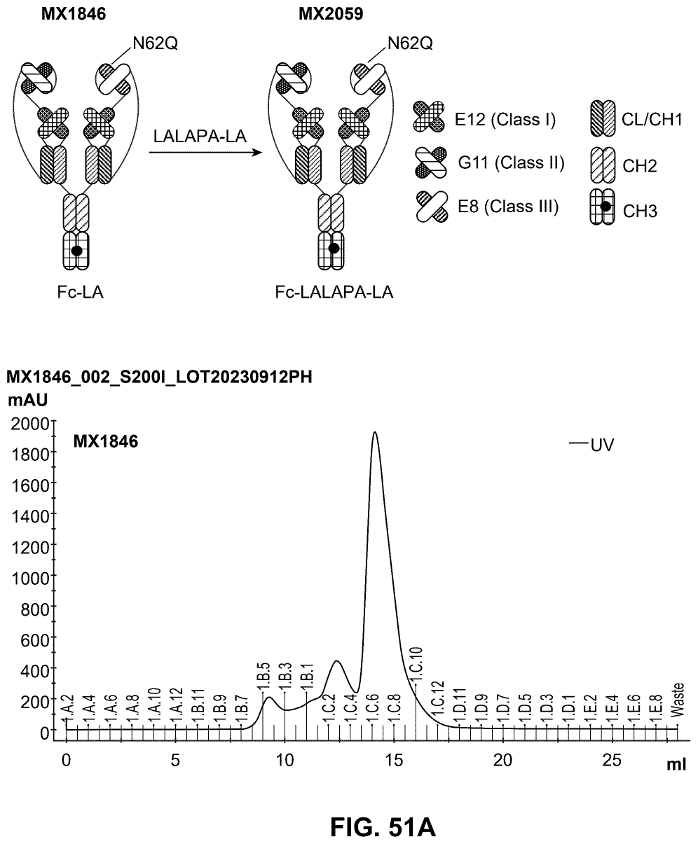

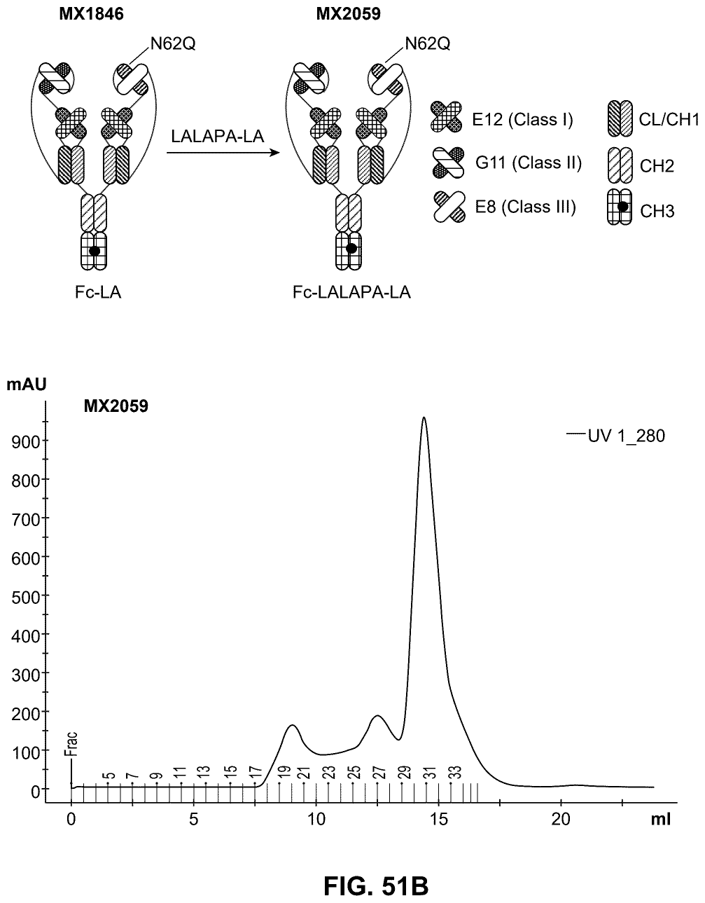

Size exclusion chromatography (SEC), using superdex 200 increase column, and 10 mM Histidine pH 6.0 and 150 mM NaCl as running buffer, was performed on MX1846 and MX2059. The results are shown in FIGS. 51A-51B.

The antigen binding polypeptide complexes were expressed in ExpiCHO-S culture and titer was measured by Octet BLI using protein A biosensor or yield from affinity chromatography (protein A). The results are shown in Table E22.

TABLE E22
MX1846, MX2005, and MX2059 expression.

| Name | Titer (mg/L) |
|---|---|
| MX1846 | 61.6 |
| MX2005 | 51.5 |
| MX2059 | 45 |

The neutralizing activity of MX1846 and MX2059 was also measured, and the results are shown in Table E23.

TABLE E23
Neutralizing activities comparison between MX1846 and
MX2059. IC50 and IC80 are expressed in nM.

| | XBB.1.5 | | EG.5 | | JN.1 | |
|---|---|---|---|---|---|---|
| Antibody | IC50 | IC80 | IC50 | IC80 | IC50 | IC80 |
| MX1846 | 0.0989 | 0.2262 | 0.2015 | 0.4408 | 11.5650 | 49.4500 |
| MX2059 | 0.2118 | 0.2427 | 0.0435 | 0.1416 | 9.8900 | 27.4300 |

### Example 17

### Linkers Manipulation and Optimization

The length of the linkers comprised in the antigen binding polypeptides and in the antigen binding polypeptide complexes disclosed herein was manipulated as described below.

Antigen binding polypeptide complex MX1545 comprises two identical antigen binding polypeptides of SEQ ID NO: 470. Each one of the two antigen binding polypeptides comprised in MX1545 has a structure of: VL1-CL-L1-VL2-L2-VH2-L3-VH1-CH1-CH2-CH3, wherein VL1 and VH1 comprise a VL and a VH of E12, respectively; and VL2 and VH2 comprise a VL and a VH of E8, respectively. In MX1545 a linker (L2) comprising 20 amino acid residues is interposed between the VL and the VH of E8. Specifically, the linker comprises an amino acid sequence of 20 amino acid residues comprising a sequence of GGGGSGGSGSGGGGSASGSG (SEQ ID NO: 340).

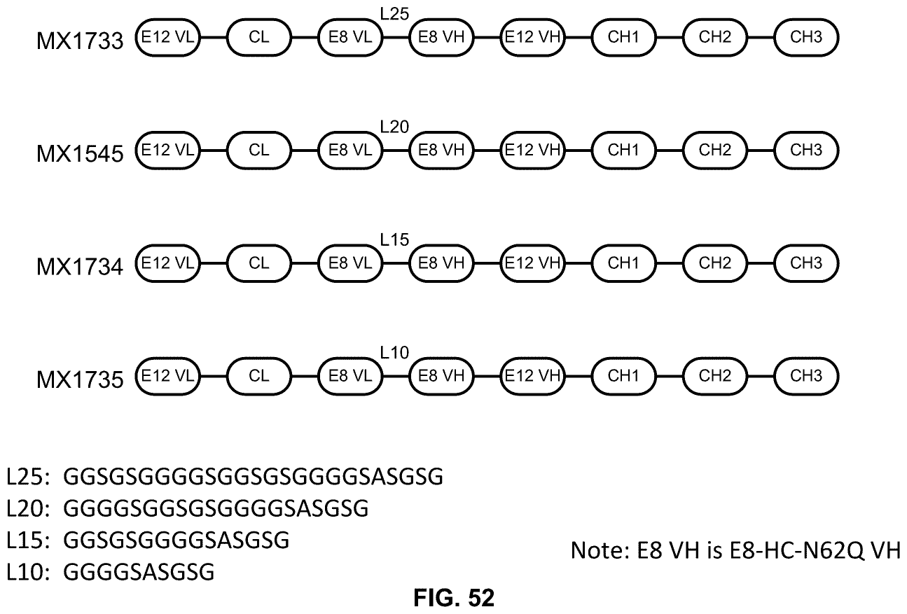

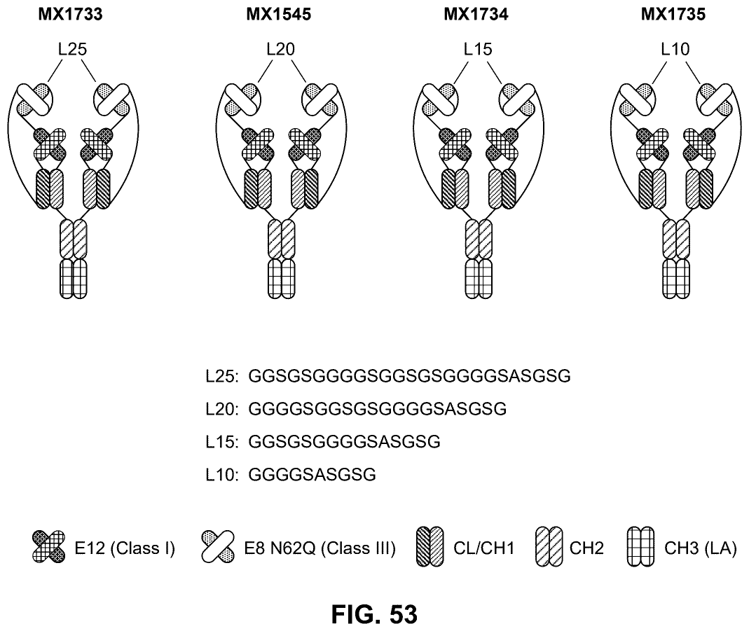

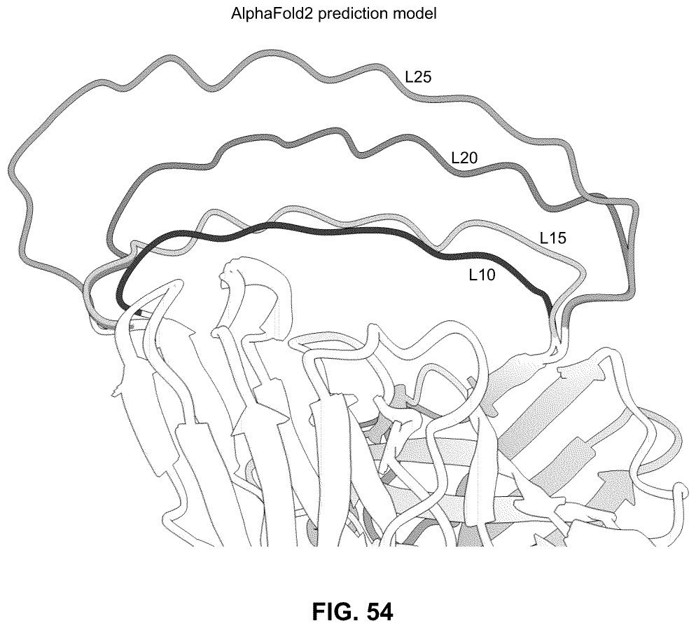

The linker interposed between the VL and the VH of E8 in MX1545 was substituted with a linker comprising 25 amino acid residues comprising a sequence of GGSGSGGGGSGGSGSGGGGSASGSG (SEQ ID NO: 967) as to obtain MX1733. Further, the linker interposed between the VL and the VH of E8 in MX1545 was substituted with a linker comprising 15 amino acid residues comprising a sequence of GGSGSGGGGSASGSG (SEQ ID NO: 337) as to obtain MX1734. Additionally, the linker interposed between the VL and the VH of E8 in MX1545 was substituted with a linker comprising 10 amino acid residues comprising a sequence of GGGGSASGSG (SEQ ID NO: 968) as to obtain MX1735, FIG. 52 shows the linear structure of the antigen binding polypeptides comprised in MX1545, MX1733, MX1734, and MX1735, FIG. 53 shows a schematic of the structures of MX1545, MX1733, MX1734, and MX1735, FIG. 54 shows a schematic of a AlphaFold2 prediction model of the 20, 25, 15, and 10 amino acid long linkers comprised in MX1545, MX1733, MX1734, and MX1735, respectively.

MX1545, MX1733, MX1734, and MX1735 were expressed in ExpiCHO-S culture and expression titers were measured. The expression titers of MX1545, MX1733, MX1734, and MX1735 are shown in Table E24.

TABLE E24
Expression titers of MX1545, MX1733, MX1734, and MX1735.

| ID | Linker | Titer (mg/L) |
|---|---|---|
| MX1735 | L10 | 3.8 |
| MX1734 | L15 | 13.9 |
| MX1545 | L20 | 9.9 |
| MX1733 | L25 | 9.0 |

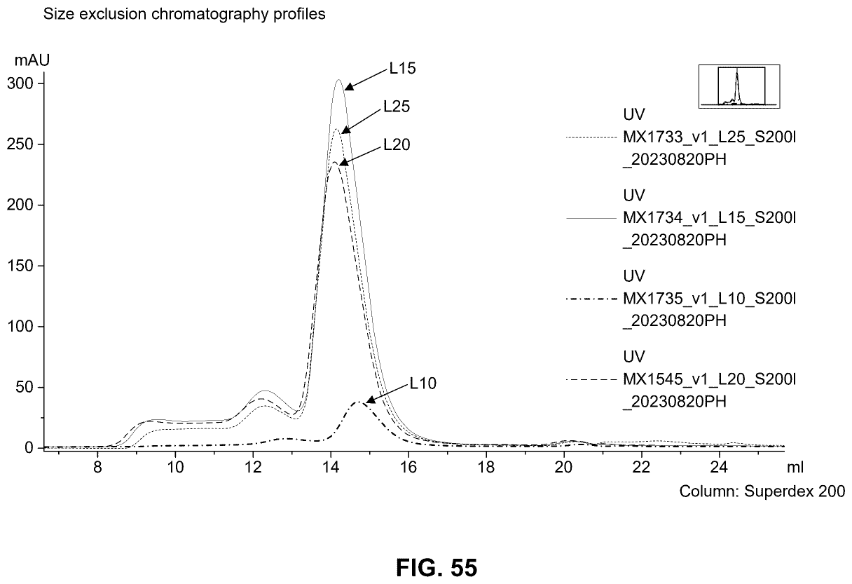

Size exclusion chromatography (SEC), using superdex 200 increase column, was performed on MX1545, MX1733, MX1734, and MX1735. The results are shown in FIG. 55.

The neutralization profile of MX1545, MX1733, MX1734, and MX1735 (IC50 and IC80) against SARS-CoV-2 (XBB.1.5) is shown in Table E25.

TABLE E25
neutralization profile of MX1545, MX1733, MX1734, and
MX1735 against SARS-CoV-2 (expressed in nM).

| | | XBB.1.5 | |
|---|---|---|---|
| ID | Linker | IC50 | IC80 |
| MX1735 | L10 | 0.54 | 1.36 |
| MX1734 | L15 | 0.20 | 0.51 |
| MX1545 | L20 | 0.17 | 0.51 |
| MX1733 | L25 | 0.15 | 0.39 |

In MX1545 a linker (L3) comprising 5 amino acid residues is interposed between the VH of E8 and the VH of E12. Specifically, the linker comprises an amino acid sequence of 5 amino acid residues comprising a sequence of GGSSG (SEQ ID NO: 333).

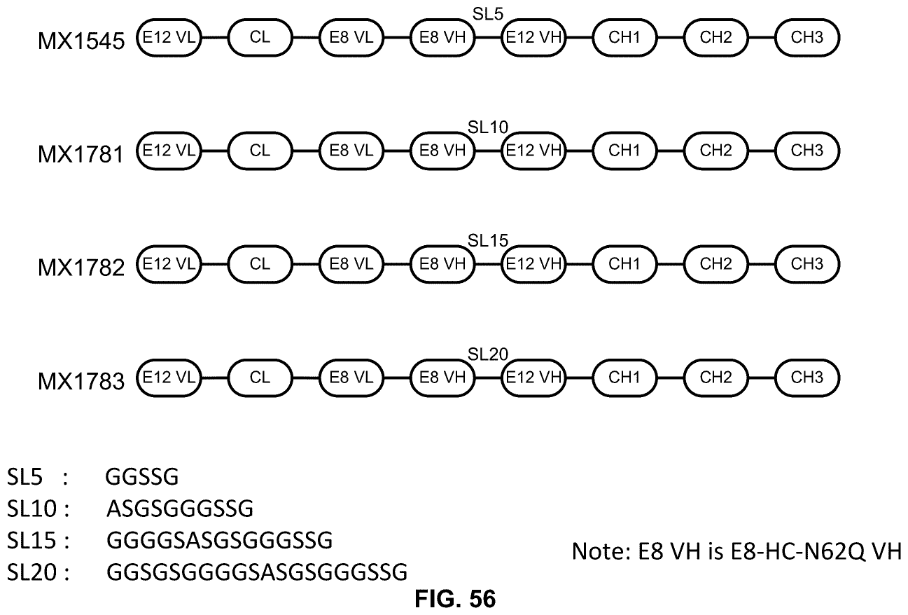

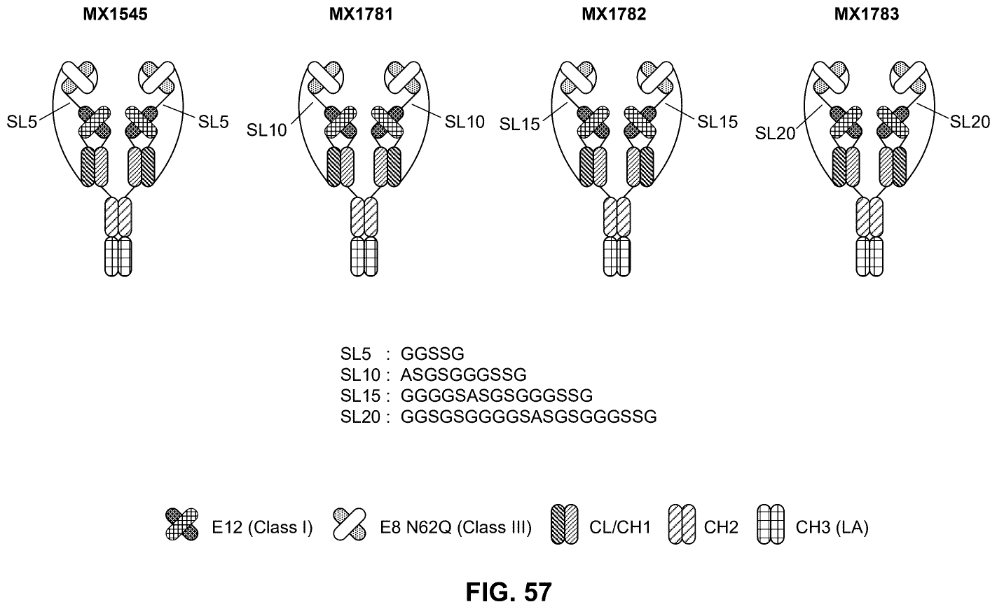

The linker interposed between the VH of E8 and the VH of E12 in MX1545 was substituted with a linker comprising 10 amino acid residues comprising a sequence of ASGSGGGSSG (SEQ ID NO: 969) as to obtain MX1781. Further, the linker interposed between the VH of E8 and the VH of E12 in MX1545 was substituted with a linker comprising 15 amino acid residues comprising a sequence of GGGGSASGSGGGSSG (SEQ ID NO: 970) as to obtain MX1782. Additionally, the linker interposed between the VH of E8 and the VH of E12 in MX1545 was substituted with a linker comprising 20 amino acid residues comprising a sequence of GGSGSGGGGSASGSGGGSSG (SEQ ID NO: 971) as to obtain MX1783, FIG. 56 shows the linear structure of the antigen binding polypeptides comprised in MX1545, MX1781, MX1782, and MX1783, FIG. 57 shows a schematic of the structures of MX1545, MX1781, MX1782, and MX1783.

MX1545, MX1781, MX1782, and MX1783 were expressed in ExpiCHO-S culture and expression titers were measured. The expression titers of MX1545, MX1781, MX1782, and MX1783 are shown in Table E26.

TABLE E26
Expression titers of MX1545, MX1781, MX1782, and MX1783.

| ID | Linker | Titer (mg/L) |
|---|---|---|
| MX1545 | SL5 | 7.05 |
| MX1781 | SL10 | 6.37 |
| MX1782 | SL15 | 8.08 |
| MX1783 | SL20 | 7.73 |

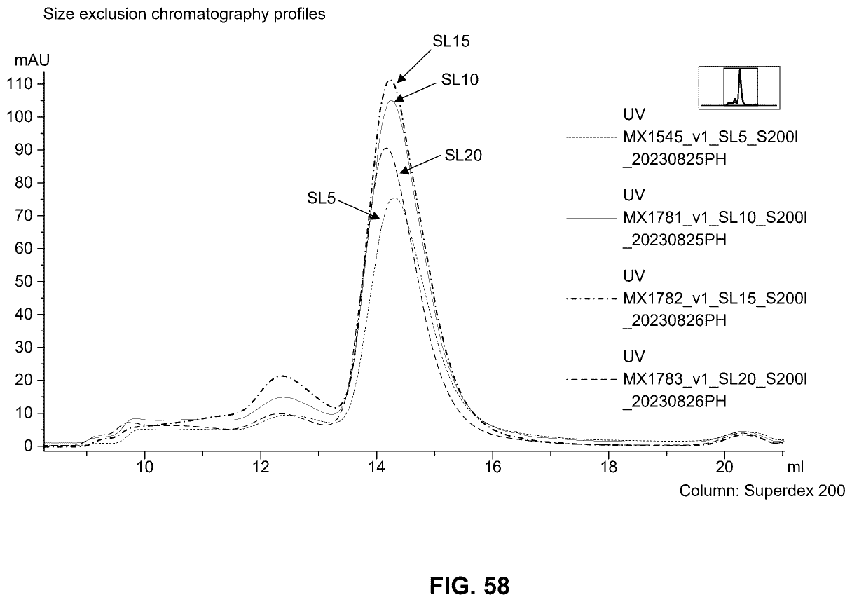

Size exclusion chromatography (SEC), using superdex 200 increase column, was performed on MX1545, MX1781, MX1782, and MX1783. The results are shown in FIG. 58.

The neutralization profile of MX1545, MX1781, MX1782, and MX1783 (IC50 and IC80) against SARS-CoV-2 (XBB.1.5) is shown in Table E27.

TABLE E27
neutralization profile of MX1545, MX1781, MX1782, and MX1783
against SARS-CoV-2 (expressed in nM).

| | | XBB.1.5 | |
|---|---|---|---|
| ID | Linker | IC50 | IC80 |
| MX1545 | SL5 | 0.19 | 0.22 |
| MX1781 | SL10 | 0.04 | 0.12 |
| MX1782 | SL15 | 0.02 | 0.14 |
| MX1783 | SL20 | 0.25 | 0.47 |

Antigen binding polypeptide complex MX1546 comprises two identical antigen binding polypeptides of SEQ ID NO: 471. Each one of the two antigen binding polypeptides comprised in MX1546 has a structure of: VL1-L1-VL2-L2-VH2-L3-VH1-CL-L4-CH1-CH2-CH3, wherein VL1 and VH1 comprise a VL and a VH of E12, respectively; and VL2 and VH2 comprise a VL and a VH of E8, respectively. In MX1546 a linker (L4) comprising 25 amino acid residues is interposed between the CL and the CH1. Specifically, the linker comprises an amino acid sequence of 25 amino acid residues comprising a sequence of GGGGSGGSGSGGGGSGGGGSASGSG (SEQ ID NO: 341).

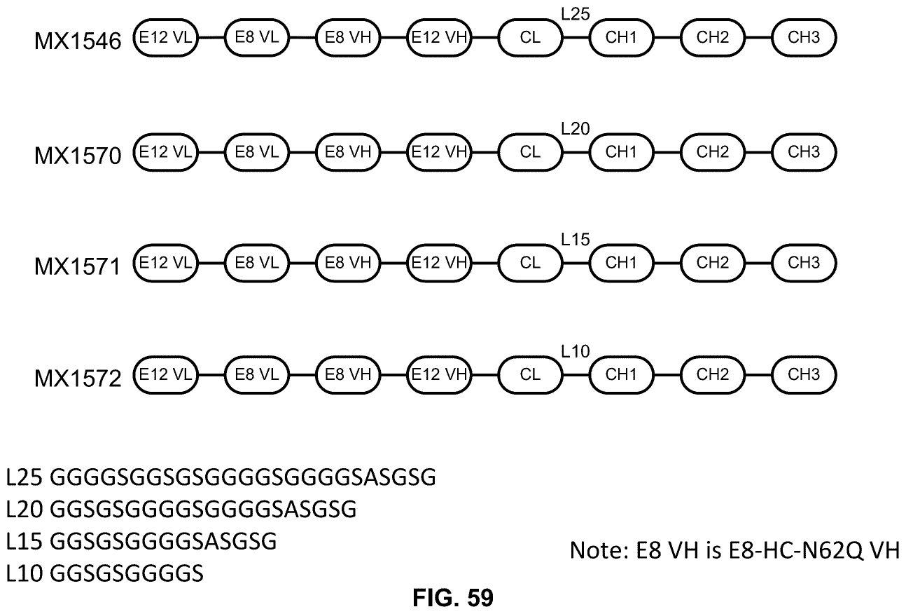

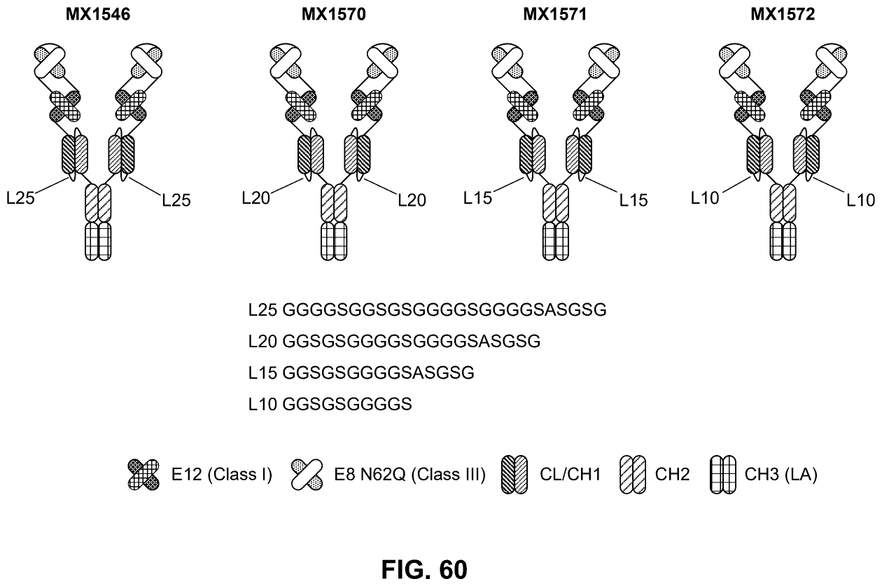

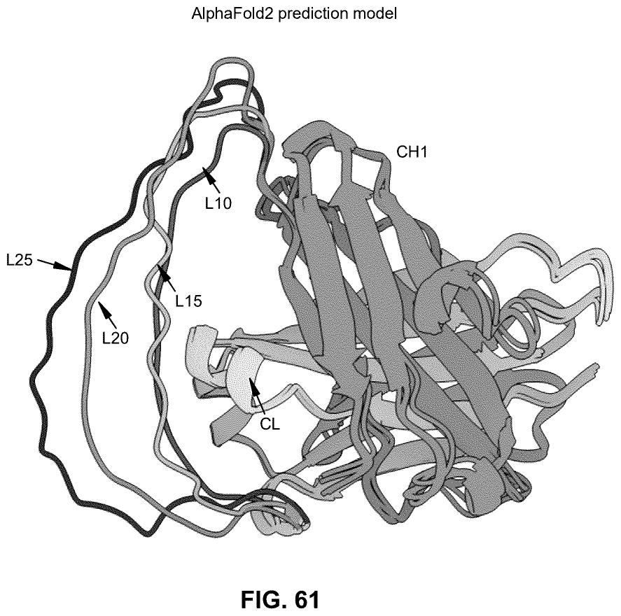

The linker interposed between the CL and the CH1 in MX1546 was substituted with a linker comprising 20 amino acid residues comprising a sequence of GGSGSGGGGSGGGGSASGSG (SEQ ID NO: 339) as to obtain MX1570. Further, the linker interposed between the CL and the CH1 in MX1546 was substituted with a linker comprising 15 amino acid residues comprising a sequence of GGSGSGGGGSASGSG (SEQ ID NO: 337) as to obtain MX1571. Additionally, the linker interposed between the CL and the CH1 in MX1546 was substituted with a linker comprising 10 amino acid residues comprising a sequence of GGSGSGGGGS (SEQ ID NO: 335) as to obtain MX1572, FIG. 59 shows the linear structure of the antigen binding polypeptides comprised in MX1546, MX1570, MX1571, and MX1572, FIG. 60 shows a schematic of the structures of MX1546, MX1570, MX1571, and MX1572, FIG. 61 shows a schematic of a AlphaFold2 prediction model of the 25, 20, 15, and 10 amino acid long linkers comprised in MX1546, MX1570, MX1571, and MX1572, respectively.

MX1546, MX1570, MX1571, and MX1572 were expressed in ExpiCHO-S culture and expression titers were measured. The expression titers of MX1546, MX1570, MX1571, and MX1572 are shown in Table E28.

TABLE E28
Expression titers of MX1546, MX1570, MX1571, and MX1572.

| ID | Linker | Titer (mg/L) |
|---|---|---|
| MX1546 | L25 | 52.5 |
| MX1570 | L20 | 35 |
| MX1571 | L15 | 18 |
| MX1572 | L10 | 19.1 |

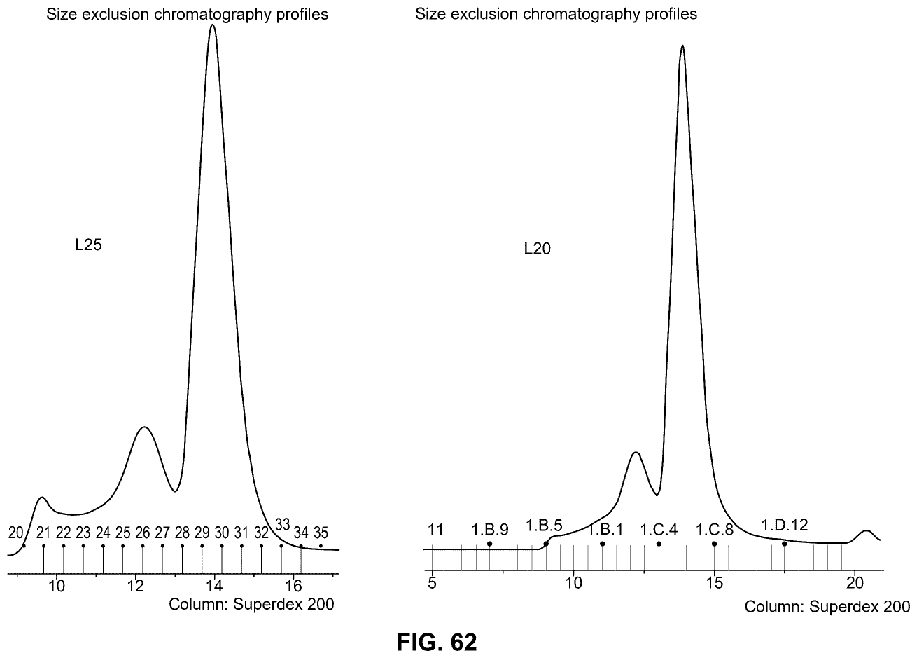

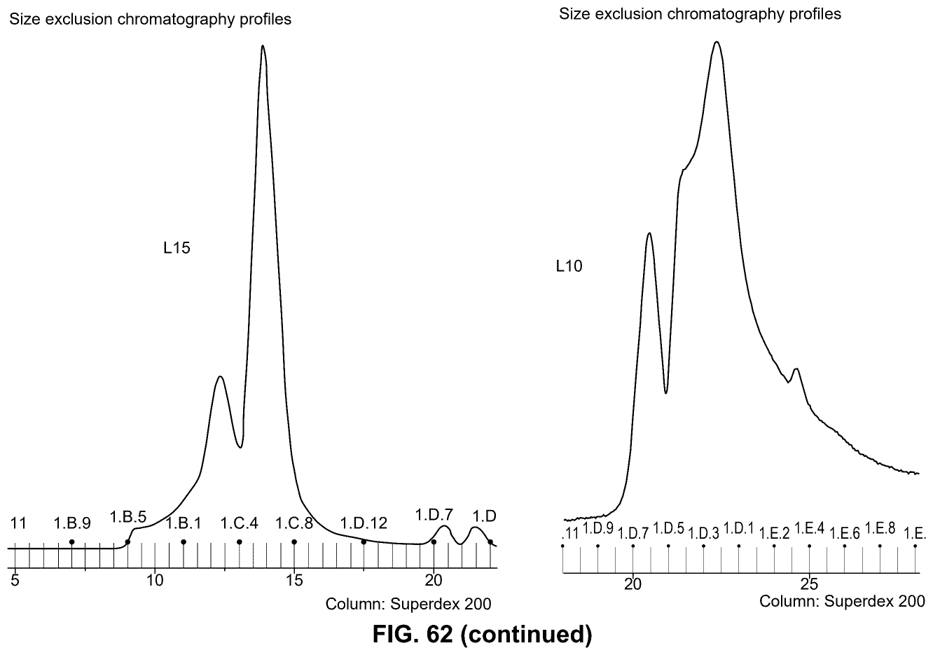

Size exclusion chromatography (SEC), using superdex 200 increase column, was performed on MX1546, MX1570, MX1571, and MX1572. The results are shown in FIG. 62.

The neutralization profile of MX1546, MX1570, MX1571, and MX1572 (IC50 and IC80) against SARS-CoV-2 (XBB.1.5) is shown in Table E29.

TABLE E29
neutralization profile of MX1546, MX1570, MX1571, and
MX1572 against SARS-CoV-2 (expressed in nM; n.t.: not tested).

| | | WA.1 | | XBB.1.5 | |
|---|---|---|---|---|---|
| ID | Linker | IC50 | IC80 | IC50 | IC80 |
| MX1546 | L25 | 0.338 | 0.376 | 0.064 | 0.176 |
| MX1570 | L20 | 0.361 | 0.405 | 0.061 | 0.150 |
| MX1571 | L15 | 0.242 | 0.450 | 0.046 | 0.083 |
| MX1572 | L10 | n.t | n.t | 0.493 | 2.725 |

Antigen binding polypeptide complex MX1544 comprises two identical antigen binding polypeptides of SEQ ID NO: 469. Each one of the two antigen binding polypeptides comprised in MX1544 has a structure of: VL1-L1-VH1-L2-VL2-CL-L3-VH2-CH1-CH2-CH3, wherein VL1 and VH1 comprise a VL and a VH of E12, respectively; and VL2 and VH2 comprise a VL and a VH of E8, respectively. In MX1544 a linker (L3) comprising 40 amino acid residues is interposed between the CL and the VH of E8. Specifically, the linker comprises an amino acid sequence of 40 amino acid residues comprising a sequence of GGGGSGGGGSGGGGSGGGGSGGGGSGGGGSGGGGSGGGGS (SEQ ID NO: 345).

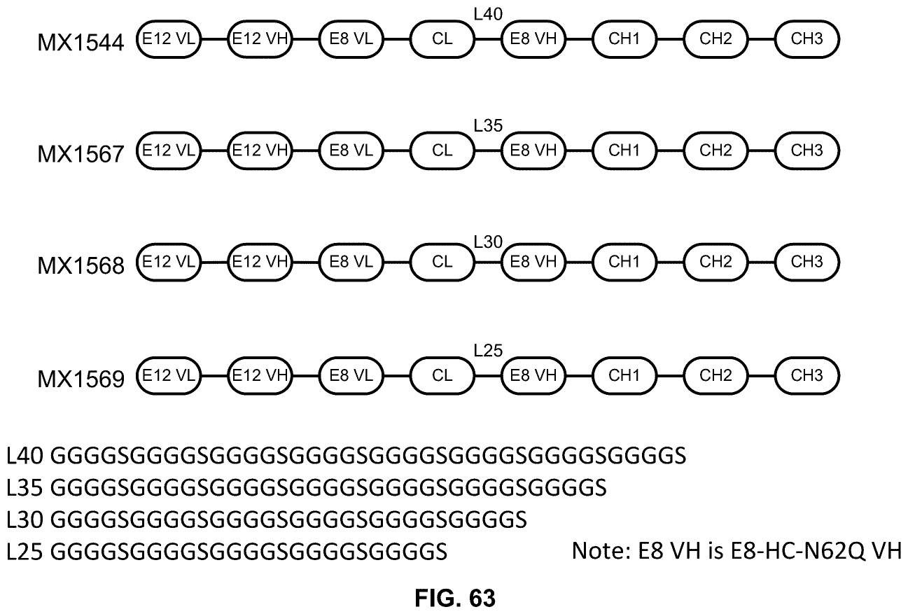

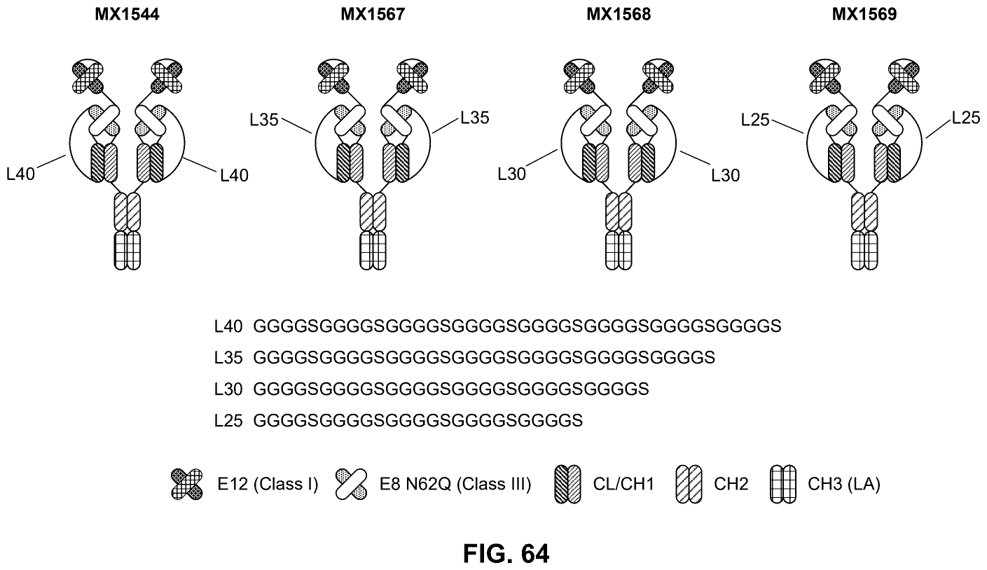

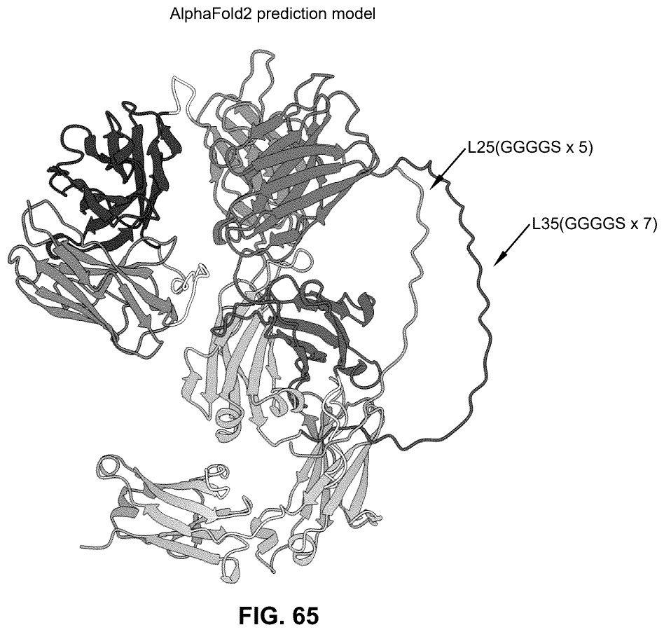

The linker interposed between the CL and the VH of E8 in MX1544 was substituted with a linker comprising 35 amino acid residues comprising a sequence of GGGGSGGGGSGGGGSGGGGSGGGGSGGGGSGGGGS (SEQ ID NO: 344) as to obtain MX1567. Further, the linker interposed between the CL and the VH of E8 in MX1544 was substituted with a linker comprising 30 amino acid residues comprising a sequence of GGGGSGGGGSGGGGSGGGGSGGGGSGGGGS (SEQ ID NO: 343) as to obtain MX1568. Additionally, the linker interposed between the CL and the VH of E8 in MX1544 was substituted with a linker comprising 25 amino acid residues comprising a sequence of GGGGSGGGGSGGGGSGGGGSGGGGS (SEQ ID NO: 342) as to obtain MX1569, FIG. 63 shows the linear structure of the antigen binding polypeptides comprised in MX1544, MX1567, MX1568, and MX1569, FIG. 64 shows a schematic of the structures of MX1544, MX1567, MX1568, and MX1569, FIG. 65 shows a schematic of a AlphaFold2 prediction model of the 35 and 25 amino acid long linkers comprised in MX1567 and MX1569, respectively.

MX1544, MX1567, MX1568, and MX1569 were expressed in ExpiCHO-S culture and expression titers were measured. The expression titers MX1544, MX1567, MX1568, and MX1569 are shown in Table E30.

TABLE E30
Expression titers of MX1544, MX1567, MX1568, and
MX1569 (n.t.: not tested).

| | | Titer (mg/L), | Titer (mg/L) |
|---|---|---|---|
| ID | Linker | experiment 1 | Experiment 2 |
| MX1544 | L40 | 9.3 | 13.8 |
| MX1567 | L35 | 7.5 | 10.5 |
| MX1568 | L30 | 7.7 | 10.4 |
| MX1569 | L25 | 5 | n.t |

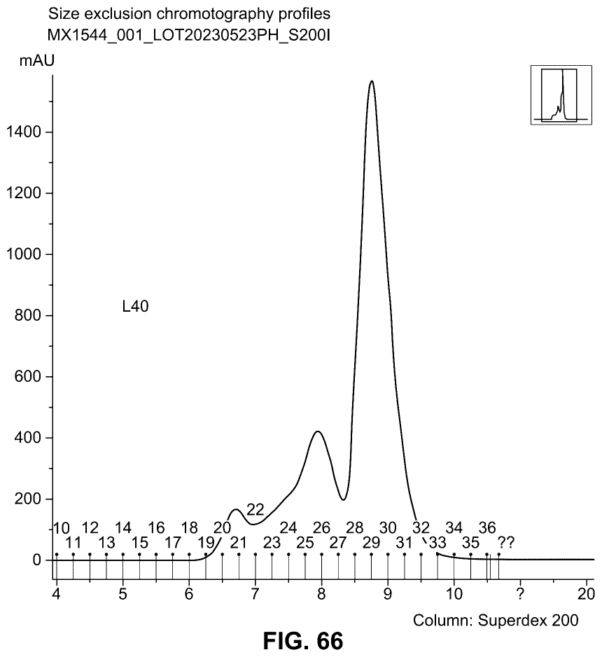

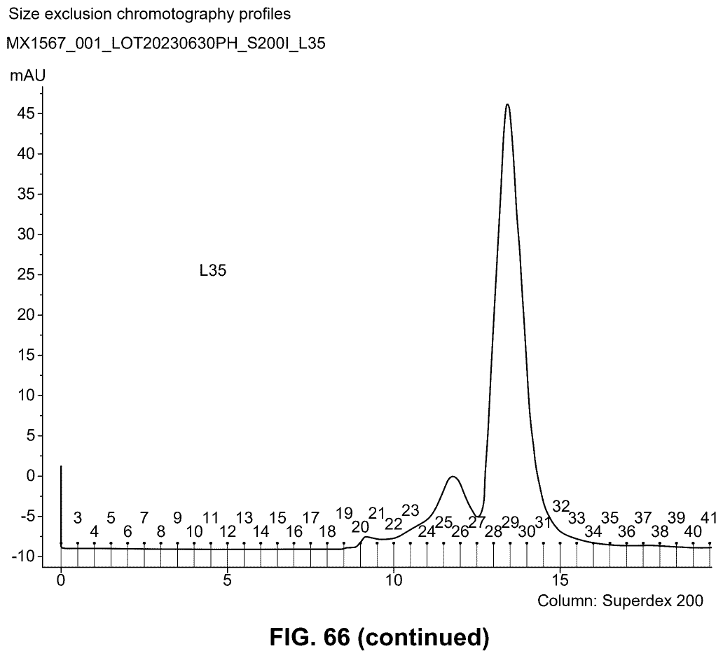

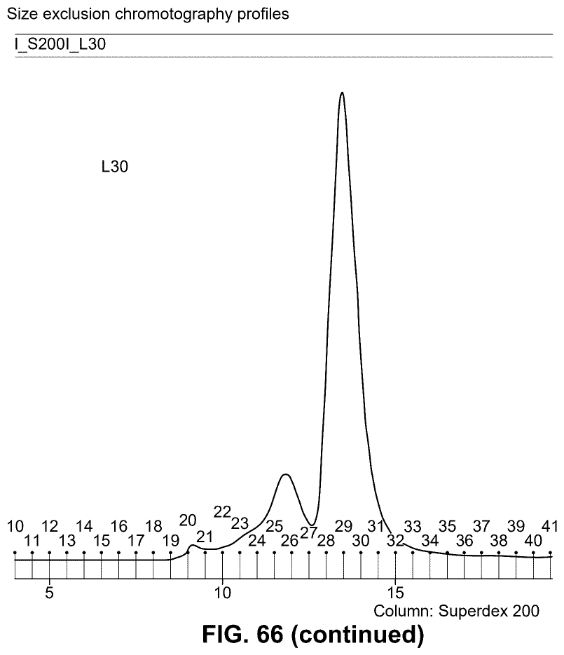

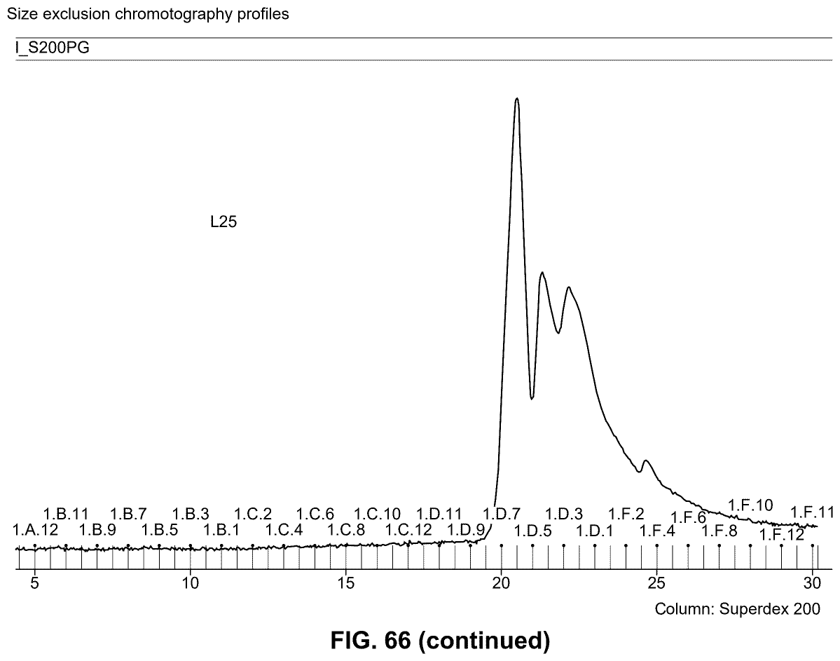

Size exclusion chromatography (SEC), using superdex 200 increase column, was performed on MX1544, MX1567, MX1568, and MX1569. The results are shown in FIG. 66.

The neutralization profile of MX1544, MX1567, MX1568, and MX1569 (IC50 and IC80) against SARS-CoV-2 (XBB.1.5) is shown in Table E31.

TABLE E31
neutralization profile of MX1544, MX1567, MX1568, and
MX1569 against SARS-CoV-2 (expressed in nM; n.t.: not tested).

| | | WA.1 | | XBB.1.5 | |
|---|---|---|---|---|---|
| ID | Linker | IC50 | IC80 | IC50 | IC80 |
| MX1544 | L40 | 0.056 | 0.377 | 0.008 | 0.022 |
| MX1567 | L35 | 0.199 | 0.313 | 0.019 | 0.046 |
| MX1568 | L30 | 0.169 | 0.232 | 0.012 | 0.032 |
| MX1569 | L25 | n.t | n.t | 0.493 | 2.555 |

### Example 18

### Neutralization Activity, Mechanism of Action, and Characterization of MX2148

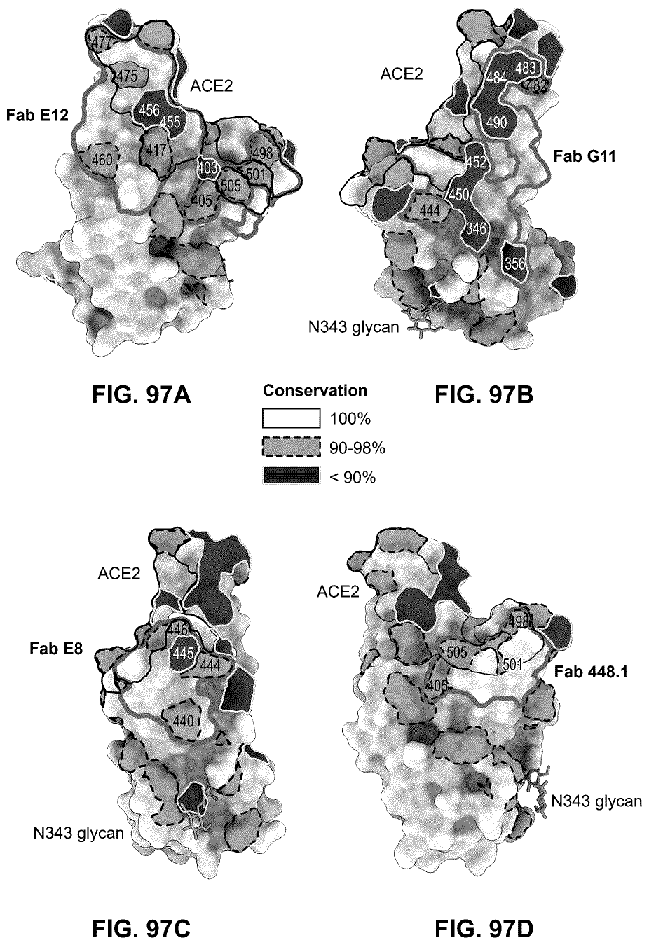

MX2148 (tetravalent tetraspecific) includes four classes of RBD targeting monoclonal antibodies, FIG. 97A-97D show cryo-EM structures of Fab binding sites of E12, E8, A18.448.1, and G11 monoclonal antibodies on the RBD. The data were generated from cryo-EM structures of spike proteins complexed with the different Fabs. Specifically, the E12 (FIG. 97A) and E8 (FIG. 97C) structures are derived from cryo-EM structures of XBB spike protein in complex with Fab E8 and Fab E12 (map at 3.4 Å resolution), the G11 structure (FIG. 97B) is derived from cryo-EM structure of XB.1.5 spike in complex with Fab G11 (map at 3.43 Å resolution), and the A18.448 structure (FIG. 97D) is derived from cryo-EM structure of EG.5 spike in complex with Fab A18.448 (Map at 3.59 Å resolution).

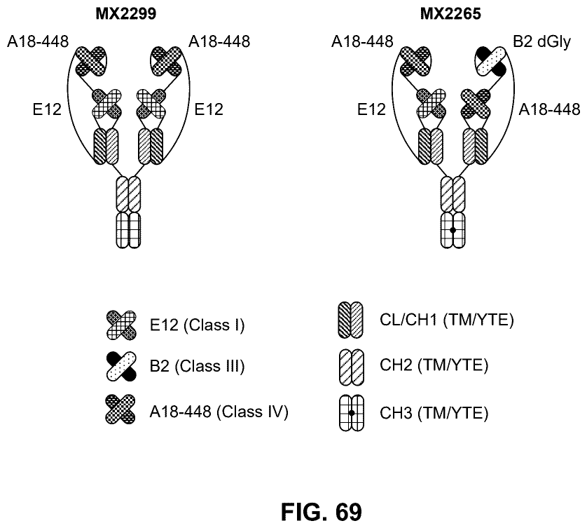

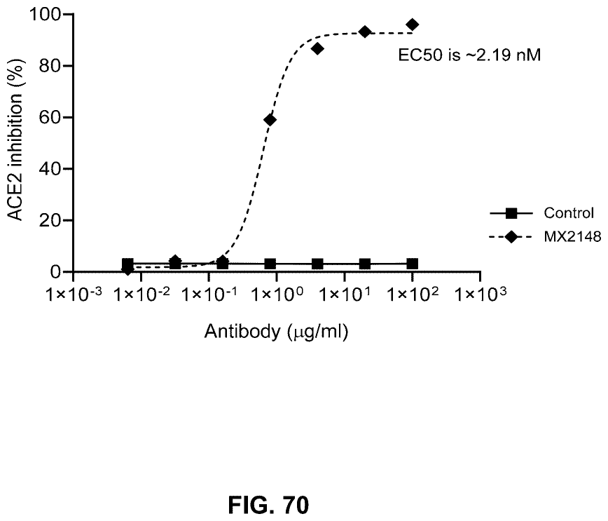

The mechanism of action and neutralization activity of MX2148 (tetravalent tetraspecific) were investigated. The data obtained showed that MX2148 blocks ACE2 binding on the SARS-CoV-2 spike protein to prevent viral entry (FIG. 70).

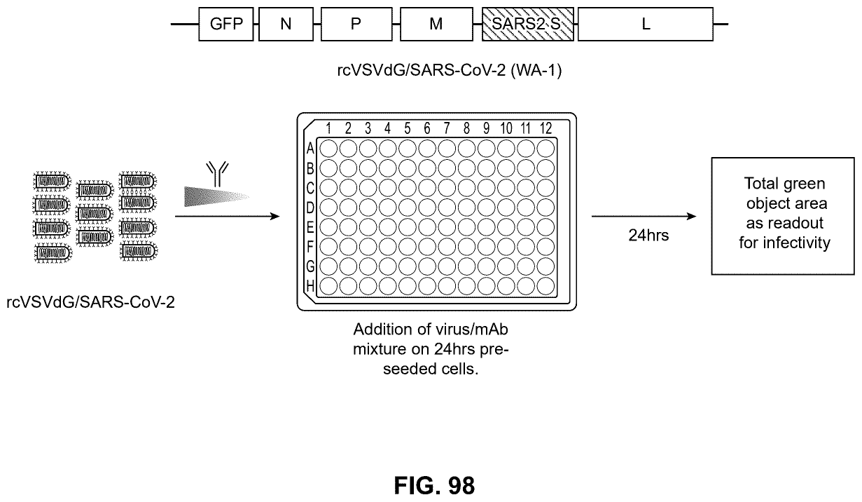

An rcVSV neutralization assay for MX2148, its constituent mAbs, and corresponding cocktail was performed (FIG. 98). The cocktail (E12+G11+E8+448.1) was made in 1:1:1:1 ratio. For a final concentration of 20 μg/ml, each individual mAb was at a concentration of 5 μg/ml.

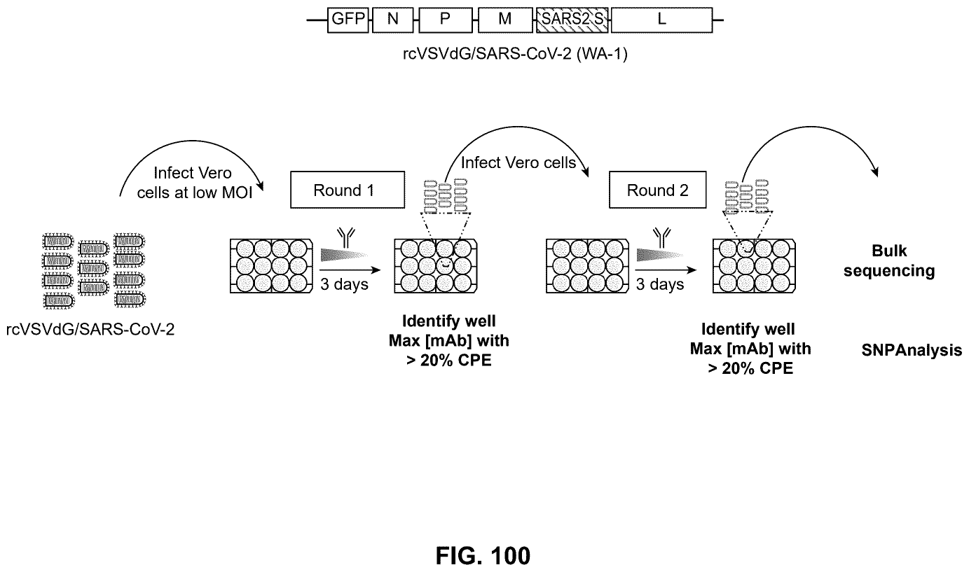

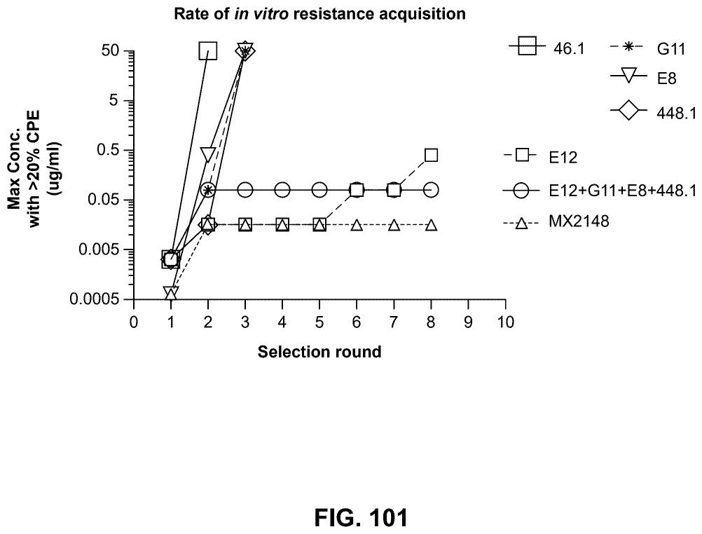

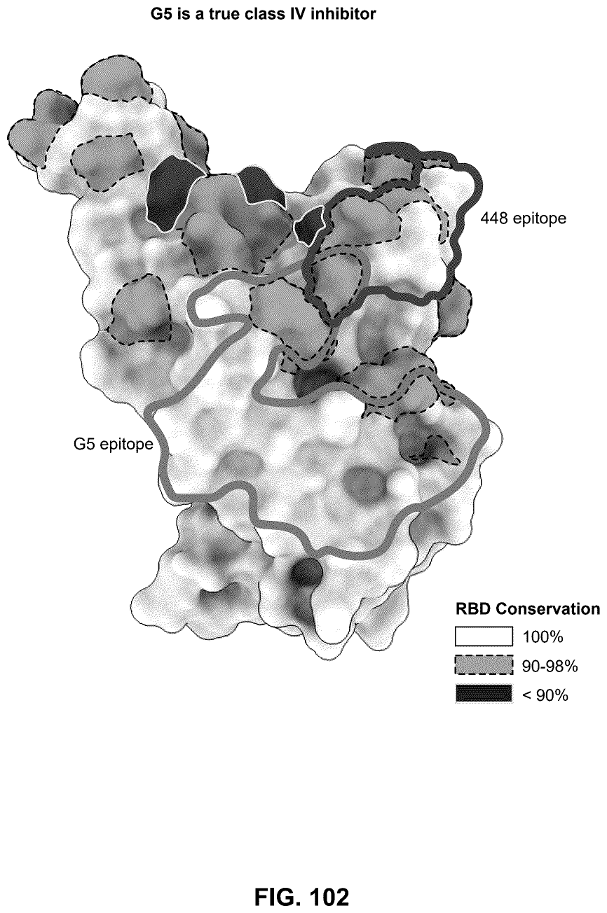

rcVSV selection of anti-SARS-Cov-2 antibodies escape mutants is shown in FIG. 100. Neutralization potency did not seem to have an impact on prevention of variant generation in individual mAbs. The ability of E12 to prevent development of resistance could be inherent in its epitope or random, MX2148 was better at prevention of variant generation than its constituent cocktails and individual monoclonal antibodies (FIG. 101. Table E32).

TABLE E32
IC50 of indicated antibodies.

| mAb | IC50 (ng/ml) |
|---|---|
| E8 | 1.0 |
| MDX2202 | 1.2 |
| 448.1 | 1.5 |
| G11 | 2.4 |
| E12 | 2.6 |
| 46.1 | 3.8 |
| E12 + G11 + E8 + 448.1 | 7.2 |

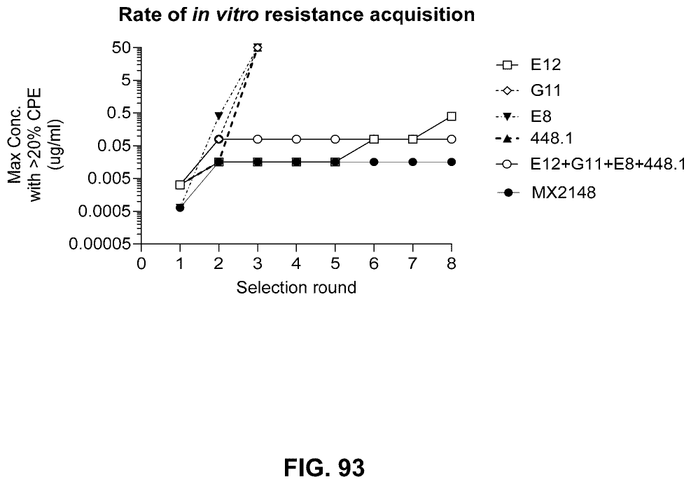

The rate of in vitro resistance acquisition of SARS-Cov-2 virus in presence of MX2148, of E12, G11, E8, A18-448, and of a mixture of E12, G11, E8, and A18-448 was measured. The virus fully escaped monoclonal antibodies E8, G11, and A18-448 (20% CPE at ˜50 mg/ml) within 2-3 rounds. Escape to E12 monoclonal antibody was delayed, with partial escape after 6 rounds, MX2148 prevented viral escape more effectively than the single monoclonal antibodies and also more effectively than the mixture of the single monoclonal antibodies (FIG. 93).

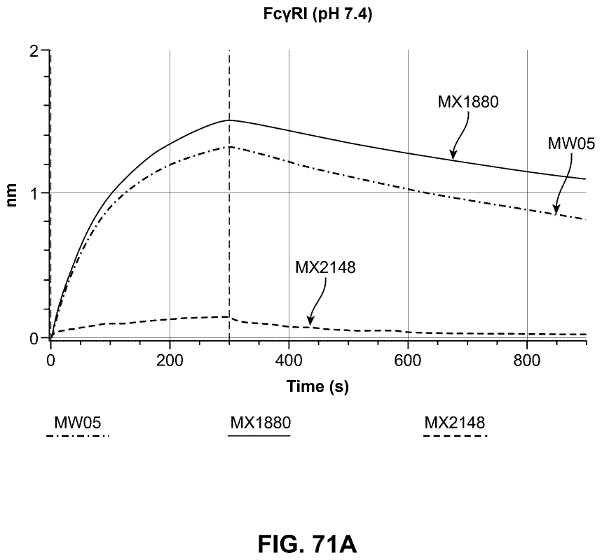

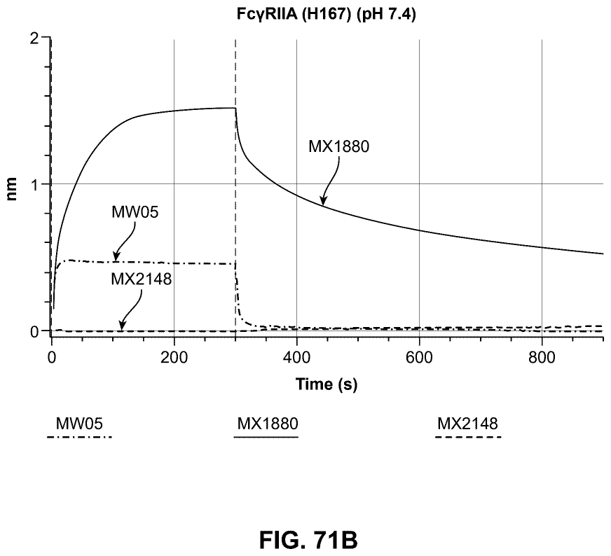

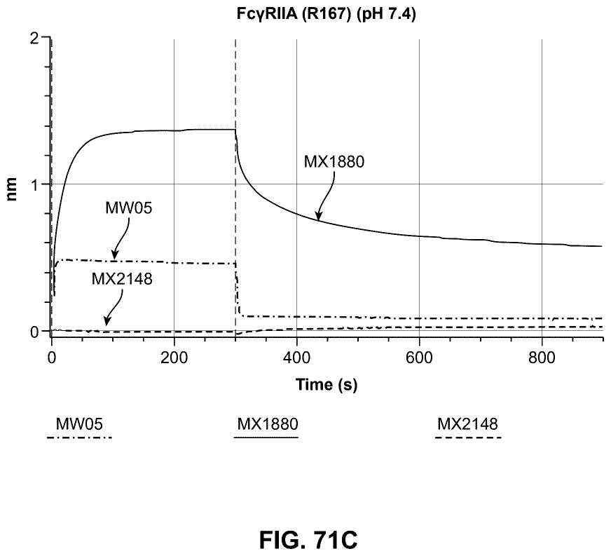

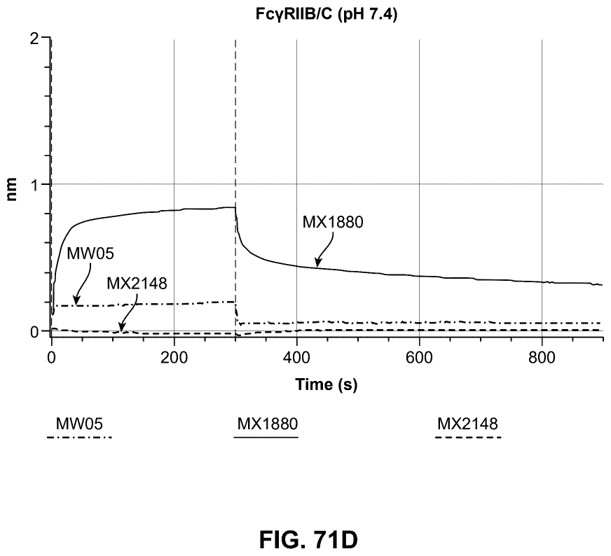

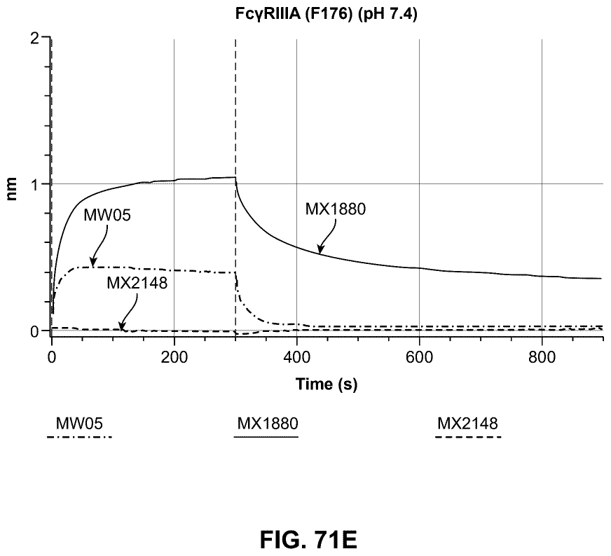

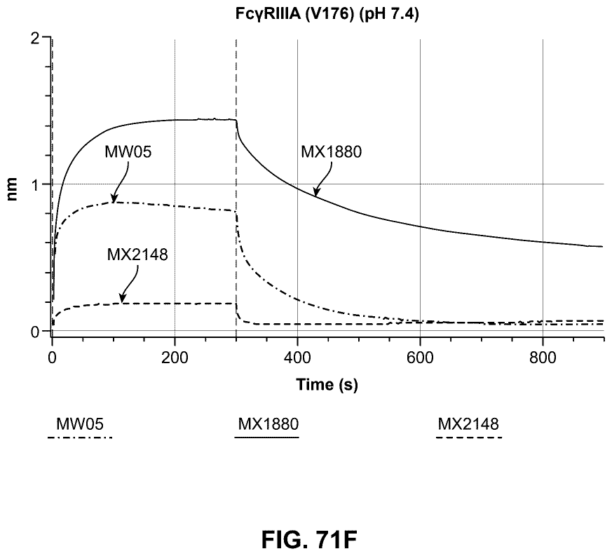

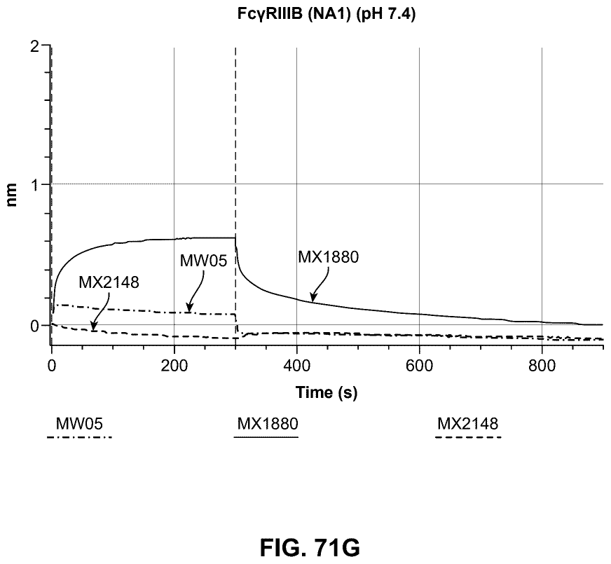

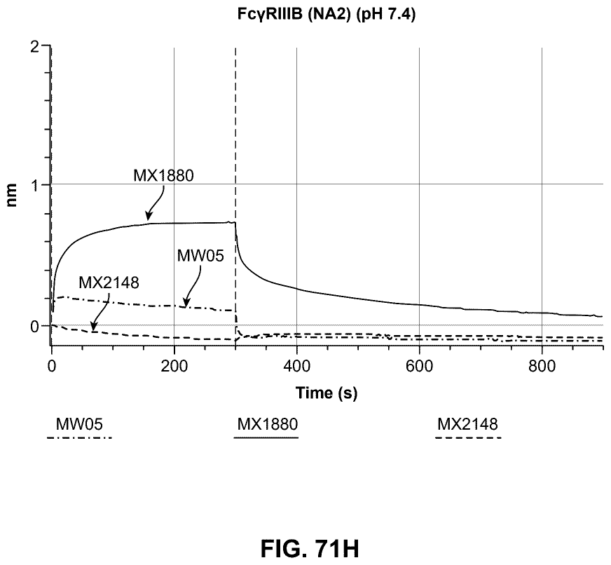

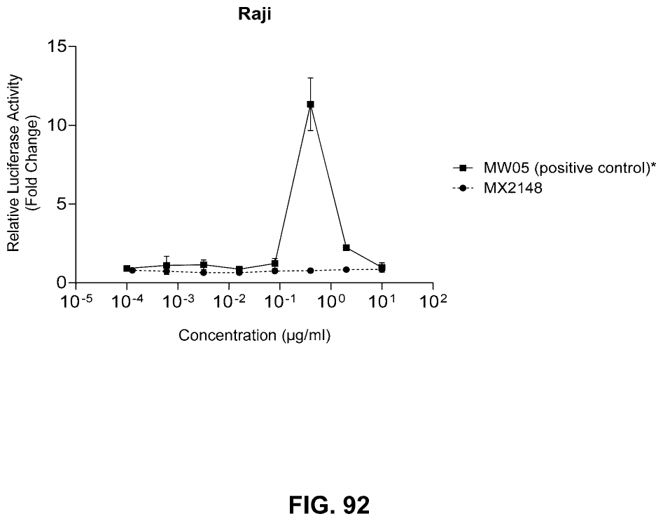

MX1880 and MX2148 are tetravalent and tetraspecific antigen binding complexes comprising, the VL and the VH of: E12 (class I), A18-448 (class I/IV), E8 (class III), and G11 (class II), wherein E8 comprises a N62Q modification, MX1880 comprises a CL/CH1 region and an Fc region (CH2-CH3) comprising a LA (M428L/N434A) modification, MX2148 comprises a CL/CH1 region and an Fc region (CH2-CH3) comprising a YTE (M252Y/S254T/T256E) and a TM (L234F/L235E/P331S) modification. The YTE (M252Y/S254T/T256E) modification is a half-life extension modification, and the TM (L234F/L235E/P331S) modification is a Fc inactivation modification. The Fc-γ function of MX1880 and MX2148 were measured and compared (FIG. 71A-71). The data obtained showed that the Fc mutations of MX2148 eliminated its Fc-γ function. Additionally, the Antibody-Dependent Enhancement (ADE) of MX2148 was tested, ADE is a phenomenon in which binding of a virus to suboptimal antibodies enhances its entry into host cells, followed by its replication. No ADE was observed with MX2148. There was no enhanced viral entry to Raji B cells at any concentration with MX2148 (FIG. 92).

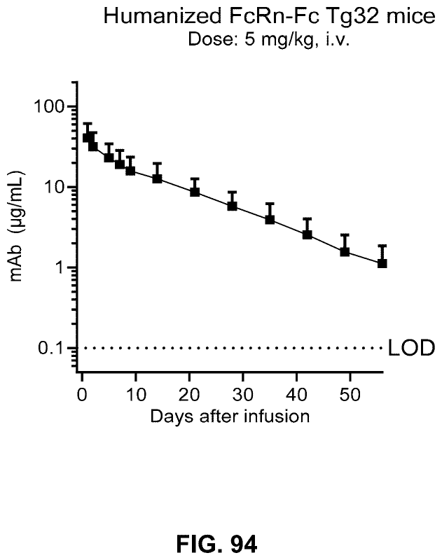

The half life of MX2148 was tested in Humanized FcRn-Fc Tg32 mice. The t½ was 12.1±1.5 days (FIG. 94). The estimated t½ in this model is comparable to standard IgG monoclonal antibodies that have been shown to have favorable pharmacokinetic profiles in humans. Other pharmacokinetic parameter estimates were as follows: Cₘₐₓ=42±22 μg/mL, AUC=544±269 day*μg/mL, and clearance=19.6±26.8 mL/day/kg.

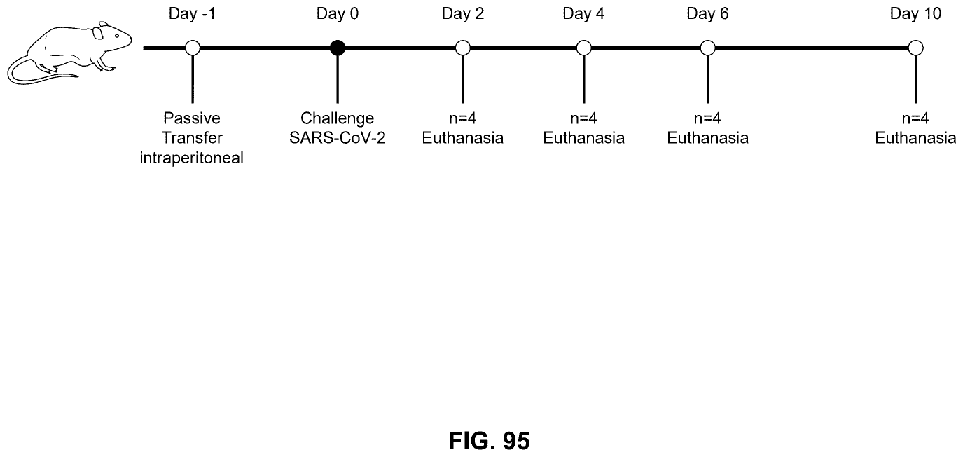

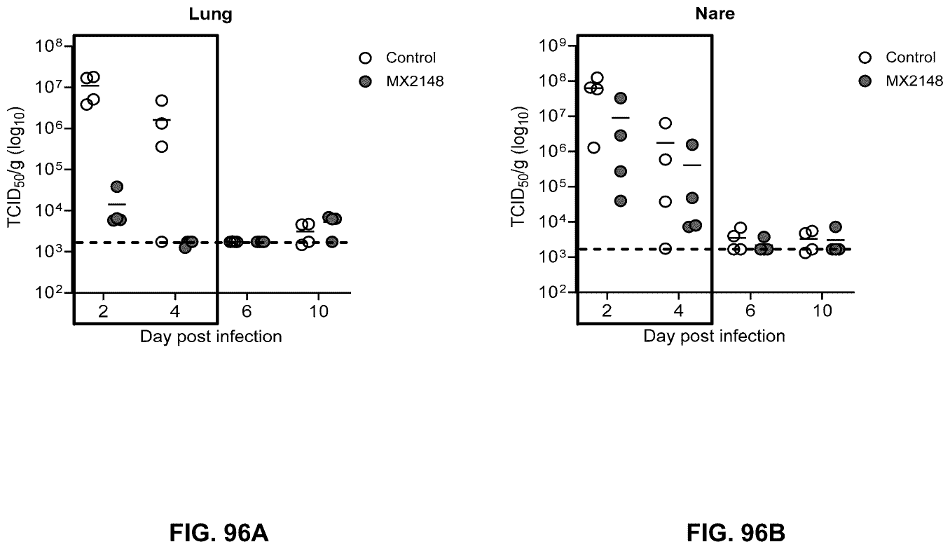

Additionally, MX2148 was shown to protect against JN.1 variant in Syrian Hamster Challenge, FIG. 95 shows the experimental design of the Syrian Hamster Challenge, and FIGS. 96A-96B show the results obtained in lung (FIG. 96A) and nare (FIG. 96B).

### Example 19

### Tetravalent Bispecific Antigen Binding Complexes

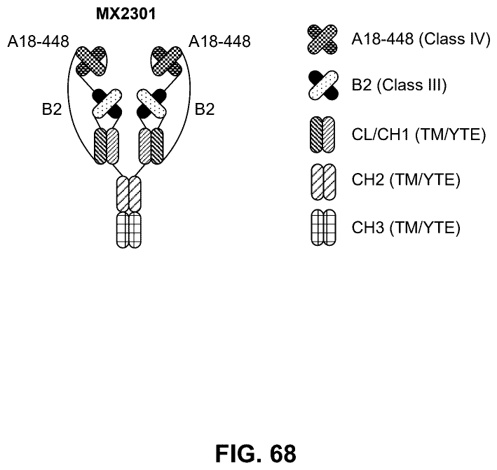

The neutralizing activity of antigen binding complex MX2301 (FIG. 68), and of monoclonal antibodies B2 and A18-448, was measured as described above, and the results are shown in Table E33. The results showed that tetravalent bispecific MX2301 potently neutralizes all variants, including KP.3.

TABLE E33
Neutralizing activities comparison between antigen binding complexes MX2301 and MX2148,
and monoclonal antibodies B2 and A18-448. IC50 and IC80 are expressed in nM.

| | | | D614G | B.1.427 | B.1.429 | P.2 | B.1.351 | B.1.1.7 | B.1.617.1 | B.1.617.2 | P.1 |
|---|---|---|---|---|---|---|---|---|---|---|---|
| Specificity | Antibody | Class (RBD) | IC50 (nM) | | | | | | | | |
| Bispecific | MX2301 | III + IV | 0.020 | 0.090 | 0.015 | 0.026 | 0.009 | 0.182 | 0.010 | 0.091 | 0.039 |
| Monoclonal | B2 | III | 0.025 | 0.309 | 0.022 | 0.041 | 0.014 | 0.387 | 0.040 | 0.124 | 0.062 |
| | A18-448 | IV | 0.039 | 0.108 | 0.077 | 0.120 | 0.038 | 0.108 | 0.070 | 0.238 | 0.078 |
| | | | B.1.526 | B.1.525 | B.1.621 | BA.1 | BA.4/5 | CH.1.1 | XBB | XBB.1.5 | |
| Specificity | Antibody | Class (RBD) | IC50 (nM) | | | | | | | | |
| Bispecific | MX2301 | III + IV | 0.014 | 0.014 | 0.032 | 0.010 | 0.011 | 0.091 | 0.022 | 0.022 | |
| Monoclonal | B2 | III | 0.025 | 0.017 | 0.120 | 0.018 | 0.039 | 3.776 | 0.256 | 0.114 | |
| | A18-448 | IV | 0.074 | 0.093 | 0.152 | 0.035 | 0.017 | 0.051 | 0.017 | 0.021 | |
| | | | XBB.2.3.2 | XBB.1.16.6 | EG.5.1 | BA.2.86 | JD.1.1 | JN.1 | JN. 1.13.1 | KP.2 | KP.3 |
| Specificity | Antibody | Class (RBD) | IC50 (nM) | | | | | | | | |
| Bispecific | MX2301 | III + IV | 0.034 | 0.025 | 0.021 | 0.016 | 0.031 | 0.023 | 0.047 | 0.033 | 0.023 |
| Monoclonal | B2 | III | 0.457 | 0.039 | 0.048 | 0.058 | 0.197 | 0.097 | 0.502 | 0.393 | 0.083 |
| | A18-448 | IV | 0.045 | 0.016 | 0.021 | 0.033 | 0.034 | 0.039 | 0.041 | 0.023 | 0.016 |

### Example 20

### Tetravalent Bispecific Antigen Binding Complexes

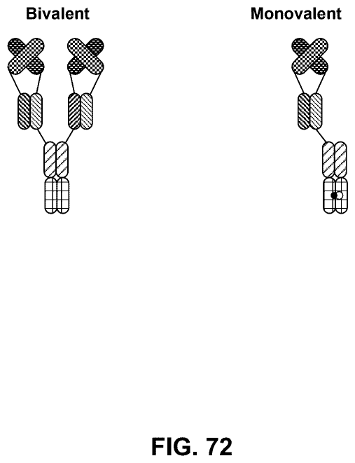

The neutralizing activity of monovalent and bivalent antigen binding complexes comprising A18-448 or B2 (FIG. 72) was measured as described above, and the results are shown in Table E34. The results showed that increased valency could improve A18-448 and B2 potency.

TABLE E34
Neutralizing activities comparison between monovalent and bivalent antigen binding
complexes comprising A18-448 or B2. IC50 and IC80 are expressed in nM.

| | Class | EG.5.1 | | JD.1.1 | | JN.1 | | KP.2 | |
|---|---|---|---|---|---|---|---|---|---|
| Antibody | (RBD) | IC50 | IC80 | IC50 | IC80 | IC50 | IC80 | IC50 | IC80 |
| 448 monovalent | IV | 0.637 | 4.406 | 1.390 | 7.198 | 0.548 | 5.165 | 0.518 | 5.005 |
| 448 bivalent | IV | 0.017 | 0.081 | 0.025 | 0.088 | 0.026 | 0.093 | 0.025 | 0.085 |
| B2 monovalent | III | 1.205 | 39.533 | 37.029 | >67 | 1.455 | 21.887 | 27.375 | >67 |
| B2 bivalent | III | 0.144 | 0.771 | 0.466 | 1.413 | 0.105 | 0.524 | 0.564 | 2.490 |

### Example 21

### Tetravalent Bispecific and Trispecific Antigen Binding Complexes

MX2299 and MX2265 were developed (FIG. 69), MX2299 is tetravalent and bispecific and comprises E12 and A18-448, MX2265 is tetravalent and trispecific and comprises E12, A18-448, and B2.

The neutralizing activity of MX2301, MX2299, MX2265, MX2242, and of monoclonal antibodies (E12, G11, E8, A18-448, B2, ADG20, and AZD3152) was measured as described above, and the results are shown in Table E35. The results showed that E12 lost activity against KP.3, while B2 and A18-448 remained active, MX2301, MX2299, MX2265, MX2242 (all multispecific) remained active against KP. 3 with sub-nanomolar IC50 potency, MX2301, MX2299, MX2265, MX2242 potently neutralized KP.3.

Bispecific MX2301 potently neutralizes all variants, including KP.3. Recent SARS-CoV-2 variants display substantial variation in class 1 binding region, MX2301 has two copies of new B2 binder (class III) and two copies of A18-448 (class IV). Both are highly potent against newer variants. The conserved nature of these class III and IV binders makes the molecules less likely to be impacted by escape mutations. Further, MX2301 showed a high titer when produced in cells.

TABLE E35
Neutralizing activities comparison between MX2301,
MX2299, MX2265, MX2242, E12, G11, E8, A18-448, B2,
ADG20, and AZD3152. IC50 and IC80 are expressed in nM.

| | | | KP.2 | KP.3 |
|---|---|---|---|---|
| Specificity | Antibody | Class (RBD) | IC50 (nM) | |
| Monoclonal | E12 | I | 4.624 | >67 |
| | G11 | II | >67 | >67 |
| | E8 | III | >67 | >67 |
| | A18-448 | IV | 0.036 | 0.017 |
| | B2 | III | 0.409 | 0.083 |
| | ADG20 | I/IV | >67 | >67 |
| | AZD3152 | I | >67 | >67 |
| Bispecific | MX2299 | I + IV | 0.048 | 0.054 |
| | MX2301 | III + IV | 0.033 | 0.023 |
| Trispecific | MX2265 | I + III + IV | 0.032 | 0.016 |
| Tetraspecific | MX2242 | I + II + III + | 0.047 | 0.041 |
| | | IV | | |

### Example 22

### Expression of MX2301 and MX2299

MX2301 (FIG. 68) and MX2299 (FIG. 69) were expressed in Expi293 cells (Life Technology). The eukaryotic expression vectors were transfected into Expi293 cells using Expi293 transfection reagent (Life Technology) as previously reported (DOI: 10.1126/science.aan8630). The transfected cells were cultured in a shaker incubator at 120 rpm. 37° C. 8% CO2 for 6 days.

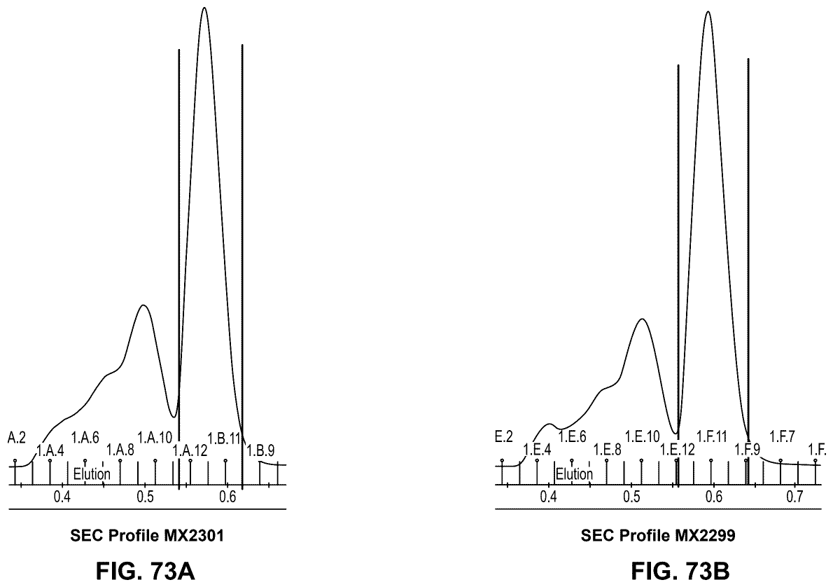

Culture supernatants were harvested and filtered, the antigen binding polypeptide complexes were purified over a Protein A (GE Health Science) column. Each antigen binding polypeptide complex was eluted with IgG elution buffer (Pierce), immediately buffer exchanged with phosphate buffered saline (PBS) and concentrated using Centricon Plus-70 (Millipore Sigma) membrane filter unit. After concentration, each antigen binding polypeptide complex was applied to a Superdex 200 16/600 size exclusion chromatography column (SEC) (Cytiva) to remove aggregates and different species in the preparation, SEC was performed using 10 mM Histidine pH 6.0 and 150 mM NaCl as running buffer, and fractions were collected and pooled in 10 mM Histidine, pH 6.0/150 mM NaCl. Expression titers were measured by Octet BLI using protein A biosensor. Size exclusion chromatography profiles for both MX2301 and MX2299 are shown in FIGS. 73A-73B, and expression titers for both MX2301 and MX2299 are shown in Table E36.

CHOK1 cell lines for stable expression of MX2301 and MX2299 were made.

TABLE E36
Production of MX2301 and MX2299 in Expi293 cells.

| | | | Yield after | Yield after |
|---|---|---|---|---|
| | Isoelectric | | protein A | SEC |
| ID | Point | Cells | (mg/L) | (mg/L) |
| MX2299 | 6.68 | Expi293 | 34.35 | 11.01 |
| MX2301 | 7.30 | Expi293 | 41.90 | 18.55 |

### Example 23

### Neutralization Activity of MX2301 and MX2299

The neutralizing activity of antigen binding complexes MX2301 (FIG. 68) and MX2299 (FIG. 69) was measured as described above, and the results are shown in Table E37.

TABLE E37
Neutralizing activities of MX2301 and MX2299. IC50 and IC80 are expressed in nM.

| nM | | | D614G | B.1.427 | B.1.429 | B.1.351 | B.1.1.7 | B.1.617.1 | B.1.617.2 | P.1 |
|---|---|---|---|---|---|---|---|---|---|---|
| Specificity | Antibody | Class (RBD) | IC50 | IC50 | IC50 | IC50 | IC50 | IC50 | IC50 | IC50 |
| Bispecific | MX2299 | I + IV | 0.027 | 0.137 | 0.023 | 0.009 | 0.215 | 0.021 | 0.128 | 0.03 |
| | MX2301 | III + IV | 0.02 | 0.09 | 0.015 | 0.009 | 0.182 | 0.01 | 0.091 | 0.039 |
| nM | | | B.1.526 | B.1.525 | B.1.621 | BA.1 | BA.4/5 | CH.1.1 | XBB | XBB.1.5 |
| Specificity | Antibody | Class (RBD) | IC50 | IC50 | IC50 | IC50 | IC50 | IC50 | IC50 | IC50 |
| Bispecific | MX2299 | I + IV | 0.022 | 0.033 | 0.062 | 0.006 | 0.007 | 0.025 | 0.009 | 0.008 |
| | MX2301 | III + IV | 0.014 | 0.014 | 0.032 | 0.01 | 0.011 | 0.091 | 0.022 | 0.022 |
| nM | | | XBB.2.3.2 | XBB.1.16.6 | EG.5.1 | BA.2.86 | JD.1.1 | JN.1 | JN.1.13.1 | KP.2 |
| Specificity | Antibody | Class (RBD) | IC50 | IC50 | IC50 | IC50 | IC50 | IC50 | IC50 | IC50 |
| Bispecific | MX2299 | I + IV | 0.014 | 0.021 | 0.016 | 0.009 | 0.035 | 0.025 | 0.027 | 0.048 |
| | MX2301 | III + IV | 0.034 | 0.025 | 0.021 | 0.016 | 0.031 | 0.023 | 0.047 | 0.033 |

### Example 24

### Expression of MX2265

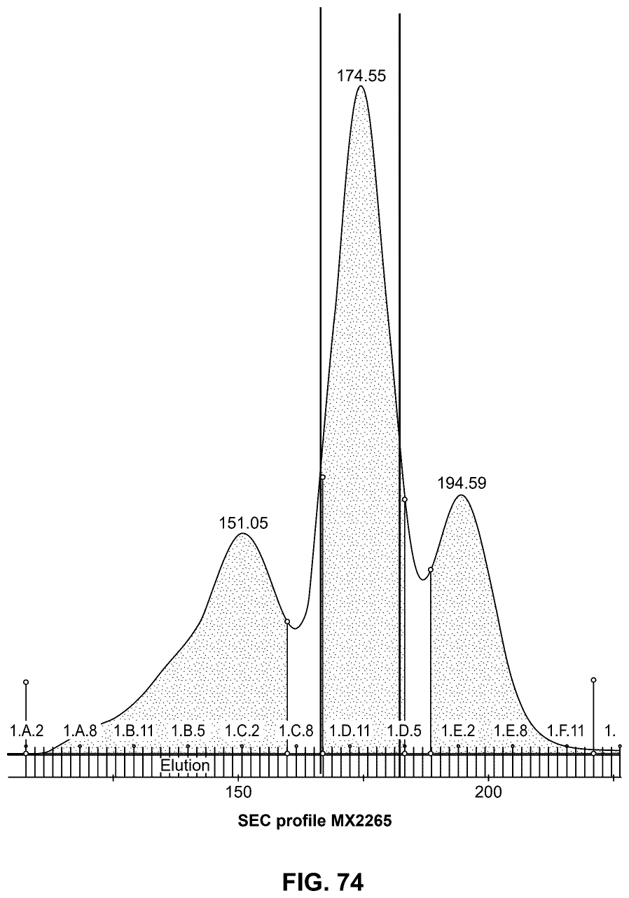

MX2265 (FIG. 69) was expressed in CHOK1 cells (stable cell line). Protein A (GE Health Science) column and Superdex 200 16/600 size exclusion chromatography column (SEC) (Cytiva), were performed as described in Example 22. Expression titers were measured by Octet BLI using protein A biosensor. Size exclusion chromatography profile for MX2265 is shown in FIG. 74, and expression titer for MX2265 is shown in Table E38.

TABLE E38
Production of MX2265 in CHOK1 cells.

| | PI | | mg/L | mg/L | |
|---|---|---|---|---|---|
| MX | Isoelectric | | Protein A | After SEC | % |
| number | point | Cell line | yield | yield | aSEC |
| MX2265 | 6.96 | CHOK1 | 181 | 72 | 93 |

### Example 25

### Neutralization Activity of MX2265

The neutralizing activity of antigen binding complex MX2265 (FIG. 69) was measured as described above, and the results are shown in Table E39.

TABLE E39
Neutralizing activities of MX2265. IC50 and IC80 are expressed in nM.

| | | nM | WA1 | | D614G | | B.1.427 | | B.1.429 | |
|---|---|---|---|---|---|---|---|---|---|---|
| Specificity | Antibody | Class (RBD) | IC50 | IC80 | IC50 | IC80 | IC50 | IC80 | IC50 | IC80 |
| Trispecific | MX2265 | I + III + IV | 0.099 | 0.304 | 0.038 | 0.102 | 0.096 | 0.244 | 0.057 | 0.105 |
| | | nM | P.2 | | B.1.351 | | B.1.1.7 | | B.1.617.1 | |
| Specificity | Antibody | Class (RBD) | IC50 | IC80 | IC50 | IC80 | IC50 | IC80 | IC50 | IC80 |
| Trispecific | MX2265 | I + III + IV | 0.059 | 0.116 | 0.02 | 0.06 | 0.075 | 0.132 | 0.071 | 0.174 |
| | | nM | B.1.617.2 | | P.1 | | B.1.526 | | B.1.525 | |
| Specificity | Antibody | Class (RBD) | IC50 | IC80 | IC50 | IC80 | IC50 | IC80 | IC50 | IC80 |
| Trispecific | MX2265 | I + III + IV | 0.244 | 0.454 | 0.066 | 0.112 | 0.055 | 0.102 | 0.07 | 0.117 |
| | | nM | B.1.621 | | BA.1 | | BA.4/5 | | CH.1.1 | |
| Specificity | Antibody | Class (RBD) | IC50 | IC80 | IC50 | IC80 | IC50 | IC80 | IC50 | IC80 |
| Trispecific | MX2265 | I + III + IV | 0.092 | 0.176 | 0.006 | 0.023 | 0.004 | 0.017 | 0.022 | 0.059 |
| | | nM | XBB | | XBB.1.5 | | XBB.2.3.2 | | XBB.1.16.6 | |
| Specificity | Antibody | Class (RBD) | IC50 | IC80 | IC50 | IC80 | IC50 | IC80 | IC50 | IC80 |
| Trispecific | MX2265 | I + III + IV | 0.018 | 0.055 | 0.008 | 0.031 | 0.037 | 0.079 | 0.031 | 0.078 |
| | | nM | EG.5.1 | | BA.2.86 | | JD.1.1 | | JN.1 | |
| Specificity | Antibody | Class (RBD) | IC50 | IC80 | IC50 | IC80 | IC50 | IC80 | IC50 | IC80 |
| Trispecific | MX2265 | I + III + IV | 0.007 | 0.04 | 0.011 | 0.045 | 0.017 | 0.08 | 0.014 | 0.051 |

| | | nM | JN.1.13.1 | | KP.2 | | KP.3 | |
|---|---|---|---|---|---|---|---|---|
| Specificity | Antibody | Class (RBD) | IC50 | IC80 | IC50 | IC80 | IC50 | IC80 |
| Trispecific | MX2265 | I + III + IV | 0.05 | 0.107 | 0.032 | 0.112 | 0.016 | 0.06 |

### Example 26

### Expression of MX2242

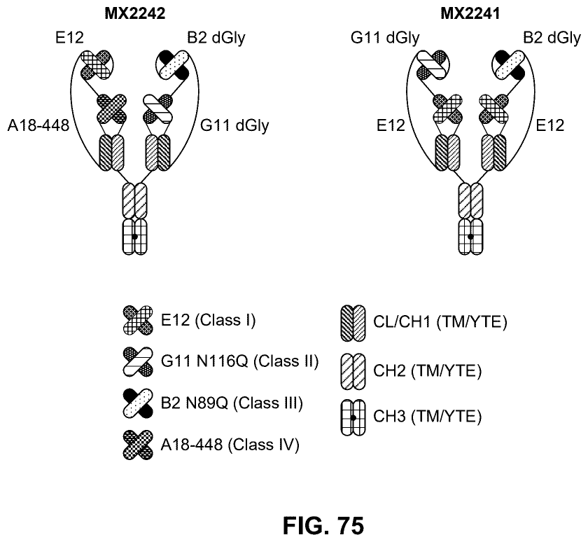

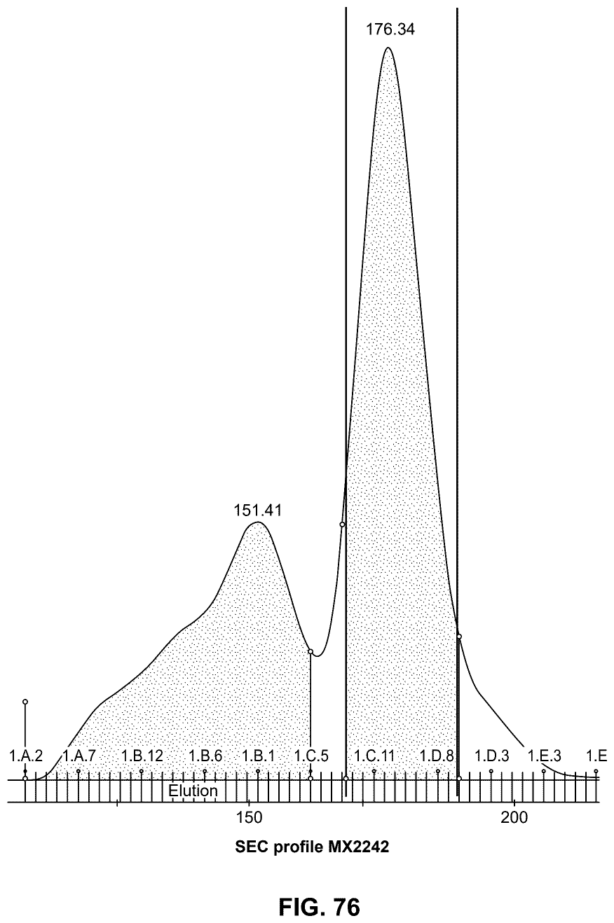

MX2242 (FIG. 43 and FIG. 75) was expressed in CHOK1 cells (stable cell line). Protein A (GE Health Science) column and Superdex 200 16/600 size exclusion chromatography column (SEC) (Cytiva), were performed as described in Example 22. Expression titers were measured by Octet BLI using protein A biosensor. Size exclusion chromatography profile for MX2242 is shown in FIG. 76, and expression titer for MX2242 is shown in Table E40.

TABLE E40
Production of MX2242 in CHOK1 cells.

| | | | mg/L | mg/L | % |
|---|---|---|---|---|---|
| MX | Isoelectric | | Protein A | After SEC | aSEC |
| number | Point | Cell line | yield | yield | results |
| MX2242 | 7.82 | CHOK1 | 252 | 106 | 98% |

### Example 27

### Neutralization Activity of MX2242

The neutralizing activity of antigen binding complex MX2242 (FIG. 43 and FIG. 75) was measured as described above, and the results are shown in Table E41.

TABLE E41
Neutralizing activities of MX2242. IC50 and IC80 are expressed in nM.

| | | nM | WA1 | | D614G | | B.1.427 | | B.1.429 | |
|---|---|---|---|---|---|---|---|---|---|---|
| Specificity | Antibody | Class (RBD) | IC50 | IC80 | IC50 | IC80 | IC50 | IC80 | IC50 | IC80 |
| Tetraspecific | MX2242 | I + II + | 0.172 | 0.614 | 0.078 | 0.13 | 0.098 | 0.423 | 0.079 | 0.129 |
| | | III + IV | | | | | | | | |
| | | nM | P.2 | | B.1.351 | | B.1.1.7 | | B.1.617.1 | |
| Specificity | Antibody | Class (RBD) | IC50 | IC80 | IC50 | IC80 | IC50 | IC80 | IC50 | IC80 |
| Tetraspecific | MX2242 | I + II + | 0.105 | 0.221 | 0.057 | 0.108 | 0.082 | 0.264 | 0.114 | 0.269 |
| | | III + IV | | | | | | | | |
| | | nM | B.1.617.2 | | P.1 | | B.1.526 | | B.1.525 | |
| Specificity | Antibody | Class (RBD) | IC50 | IC80 | IC50 | IC80 | IC50 | IC80 | IC50 | IC80 |
| Tetraspecific | MX2242 | I + II + | 0.331 | 0.509 | 0.11 | 0.208 | 0.075 | 0.126 | 0.09 | 0.141 |
| | | III + IV | | | | | | | | |
| | | nM | B.1.621 | | BA.1 | | BA.4/5 | | CH.1.1 | |
| Specificity | Antibody | Class (RBD) | IC50 | IC80 | IC50 | IC80 | IC50 | IC80 | IC50 | IC80 |
| Tetraspecific | MX2242 | I + II + | 0.159 | 0.241 | 0.009 | 0.043 | 0.003 | 0.022 | 0.048 | 0.1 |
| | | III + IV | | | | | | | | |
| | | nM | XBB | | XBB.1.5 | | XBB.2.3.2 | | XBB.1.16.6 | |
| Specificity | Antibody | Class (RBD) | IC50 | IC80 | IC50 | IC80 | IC50 | IC80 | IC50 | IC80 |
| Tetraspecific | MX2242 | I + II + | 0.024 | 0.052 | 0.02 | 0.058 | 0.051 | 0.088 | 0.059 | 0.146 |
| | | III + IV | | | | | | | | |
| | | nM | EG.5.1 | | BA.2.86 | | JD.1.1 | | JN.1 | |
| Specificity | Antibody | Class (RBD) | IC50 | IC80 | IC50 | IC80 | IC50 | IC80 | IC50 | IC80 |
| Tetraspecific | MX2242 | I + II + | 0.011 | 0.045 | 0.039 | 0.159 | 0.027 | 0.062 | 0.1 | 0.226 |
| | | III + IV | | | | | | | | |

| | | nM | JN.1.13.1 | | KP.2 | | KP.3 | |
|---|---|---|---|---|---|---|---|---|
| Specificity | Antibody | Class (RBD) | IC50 | IC80 | IC50 | IC80 | IC50 | IC80 |
| Tetraspecific | MX2242 | I + II + | 0.082 | 0.192 | 0.047 | 0.181 | 0.041 | 0.126 |
| | | III + IV | | | | | | |

### Example 28

### Expression of MX2242 and MX2241

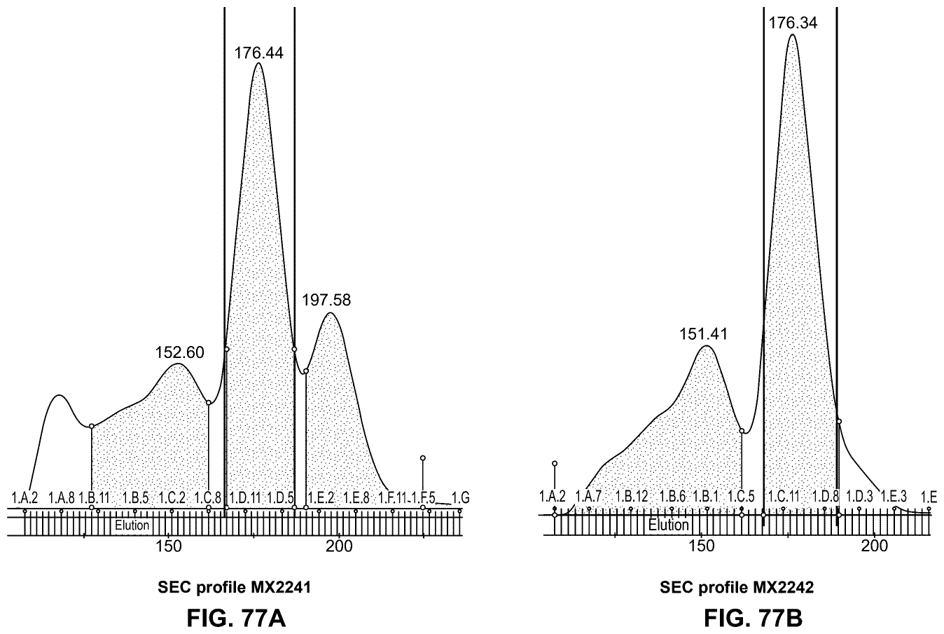

MX2242 and MX2241 (FIG. 43 and FIG. 75) were expressed in CHOK1 cells (stable cell line). Protein A (GE Health Science) column and Superdex 200 16/600 size exclusion chromatography column (SEC) (Cytiva), were performed as described in Example 22. Further MES buffer (pH6) was added after elution buffer to neutralize the pH before SEC and after Protein A purification. Expression titers were measured by Octet BLI using protein A biosensor. Size exclusion chromatography profiles for MX2242 and MX2241 are shown in FIGS. 77A-77B, and expression titers for MX2242 and MX2241 are shown in Table E42.

TABLE E42
Production of MX2242 and MX2241 in CHOK1 cells.

| | | Protein A | After MES | After SEC | Total |
|---|---|---|---|---|---|
| MX number | Cell line | yield | yield | yield | purified |
| MX2241 | CHOK1 | 252 mg/L | 255 mg/L | 84 mg/L | 21 mg |
| MX2242 | CHOK1 | 256 mg/L | 231 mg/L | 106 mg/L | 26.6 mg |

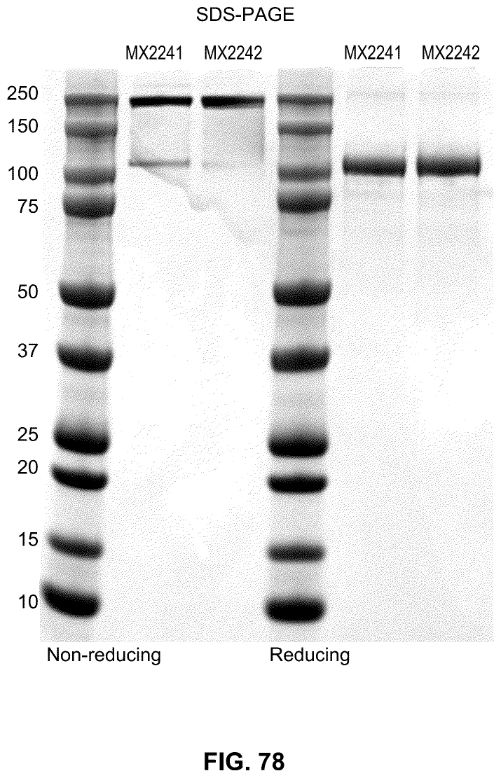

The fractions were then analyzed on reduced and non-reduced SDS-PAGE to identify the fractions that contained the antigen binding polypeptide complexes by using standard procedures, FIG. 78 shows a Western Blot analysis of the produced antigen binding polypeptide complexes.

### Example 29

### Expression of MX2263, MX2264, MX2265, MX2266, and MX2269

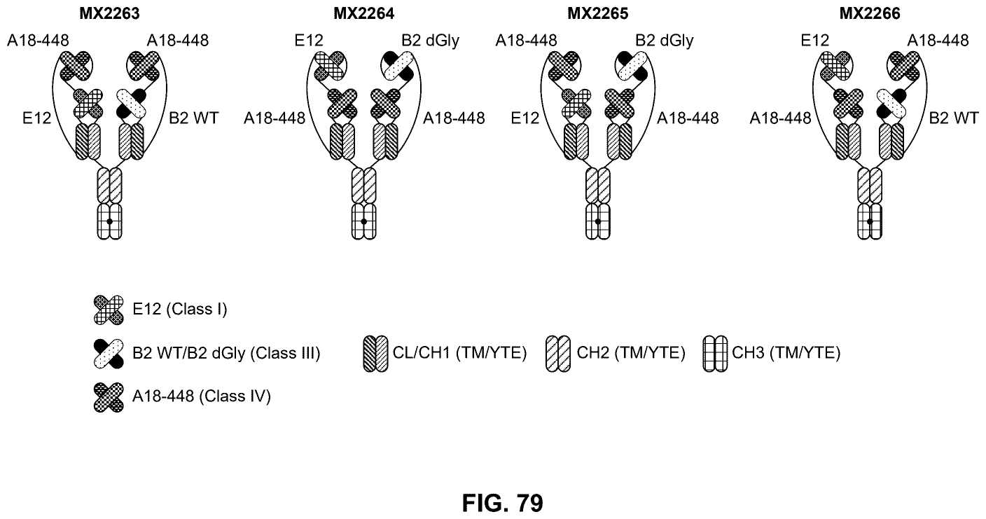

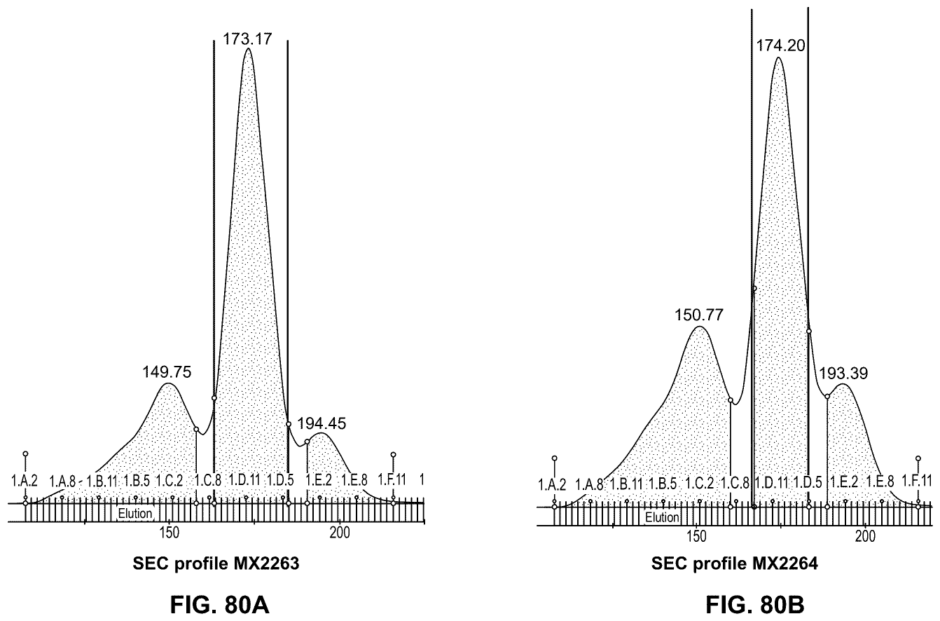

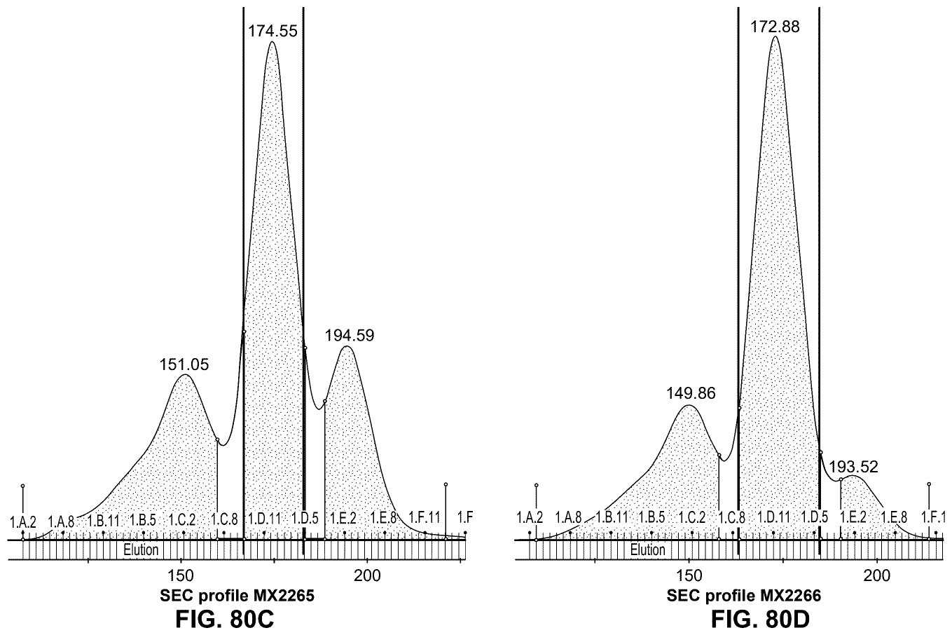

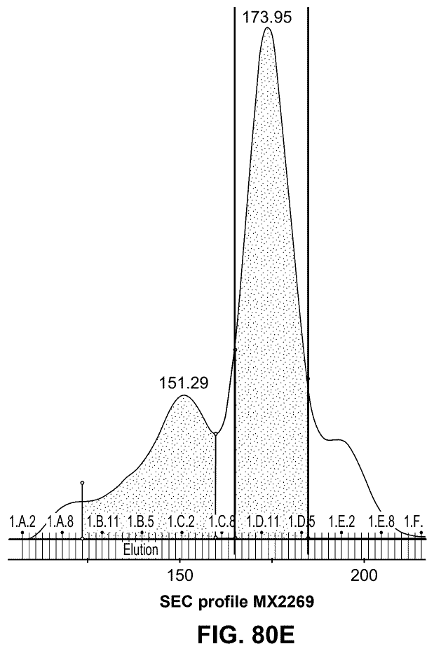

MX2263, MX2264, MX2265, MX2266, and MX2269 (FIG. 43, FIG. 69, and FIG. 79) were expressed in CHOK1 cells (stable cell line). Protein A (GE Health Science) column and Superdex 200 16/600 size exclusion chromatography column (SEC) (Cytiva), were performed as described in Example 22. Further MES buffer (pH6) was added after elution buffer to neutralize the pH before SEC and after Protein A purification. Expression titers were measured by Octet BLI using protein A biosensor. Size exclusion chromatography profiles for MX2263, MX2264, MX2265, MX2266, and MX2269 are shown in FIGS. 80A-80E, and expression titers for MX2263, MX2264, MX2265, MX2266, and MX2269 are shown in Table E43.

TABLE E43
Production of MX2263, MX2264, MX2265, MX2266, and
MX2269 in CHOK1 cells.

| | | mg/L | mg/L | mg/L | mg |
|---|---|---|---|---|---|
| | | Protein A | After MES | After SEC | Total |
| MX number | Cell line | yield | yield | yield | purified |
| MX2263 | CHOK1 | 222 | 219 | 112 | 28 |
| MX2264 | CHOK1 | 160 | 161 | 52 | 13 |
| MX2265 | CHOK1 | 181 | 184 | 72 | 18 |
| MX2266 | CHOK1 | 198 | 205 | 112 | 28 |
| MX2269 | CHOK1 | 178 | 167 | 80 | 20 |

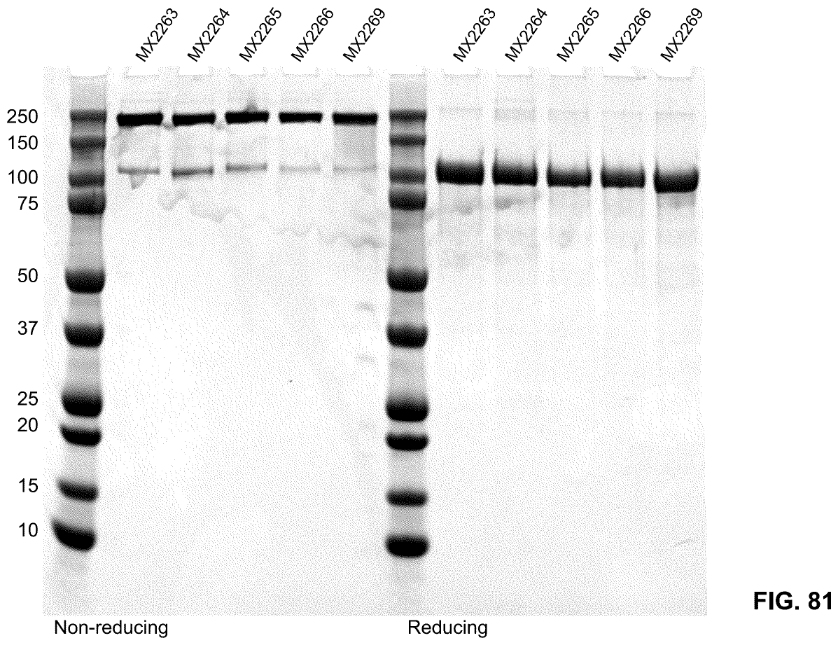

The fractions were then analyzed on reduced and non-reduced SDS-PAGE to identify the fractions that contained the antigen binding polypeptide complexes by using standard procedures, FIG. 81 shows a Western Blot analysis of the produced antigen binding polypeptide complexes.

### Example 30

### Neutralization Activity of MX2148, MX2168, MX2170, MX2242, MX2183, MX2179, MX2180, and MX2269

The neutralizing activity of antigen binding complexes MX2148, MX2168, MX2170, MX2242, MX2183, MX2179, MX2180, and MX2269 (FIG. 43) was measured as described above, and compared to the neutralizing activity of five monoclonal antigen binding proteins: MX2382 (A18-448), MX1852 (G11), MX1091 (E12), MX2270 (B2) and MX2271 (B2_N89Q). The results are shown in Table E44.

TABLE E44
Neutralizing activities of MX2148, MX2168, MX2170, MX2242, MX2183, MX2179,
MX2180, and MX2269, compared to neutralizing activity of MX2382 (A18-448), MX1852 (G11),
MX1091 (E12), MX2270 (B2) and MX2271 (B2_N89Q). IC50 and IC80 are expressed in nM.

| | | | JN.1 | | HV.1 | | Sars-Cov1 | |
|---|---|---|---|---|---|---|---|---|
| Specificity | RBD Class | Antibody | IC50 | IC80 | IC50 | IC80 | IC50 | IC80 |
| Tetraspecific | I + II + III + | MX2148 | 0.0957 | 0.3431 | 0.0341 | 0.0763 | 0.3206 | 0.865 |
| | IV | | | | | | | |
| | I + II + III + | MX2168 | 0.1756 | 0.3907 | 0.0777 | 0.1681 | 0.237 | 0.7252 |
| | IV | | | | | | | |
| | I + II + III + | MX2170 | 0.1375 | 0.3305 | 0.0458 | 0.1187 | 0.1864 | 0.525 |
| | IV | | | | | | | |
| | I + II + III + | MX2242 | 0.0997 | 0.226 | 0.0482 | 0.0895 | 0.1582 | 0.5055 |
| | IV | | | | | | | |
| | I + II + III + | MX2183 | 0.22 | 0.4965 | 0.0761 | 0.1807 | 0.5756 | 1.1888 |
| | IV | | | | | | | |
| | I + II + III + | MX2179 | 0.106 | 0.2344 | 0.1054 | 0.1901 | 0.5293 | 1.589 |
| | IV | | | | | | | |
| | I + II + III + | MX2180 | 0.1134 | 0.2478 | 0.1199 | 0.2317 | 0.4588 | 1.8313 |
| | IV | | | | | | | |
| | I + II + III + | MX2269 | 0.1079 | 0.2283 | 0.0913 | 0.1785 | 0.3419 | 1.1822 |
| | IV | | | | | | | |
| Monoclonal | IV | A18-448 | 0.0461 | 0.1162 | 0.0321 | 0.0941 | 0.0607 | 0.157 |
| | | MX2382 | | | | | | |
| | II | G11 | >67 | >67 | 4.4424 | 12.5837 | >67 | >67 |
| | | MX1852 | | | | | | |
| | I | E12 | 8.311 | 32.0835 | 5.5419 | 20.5626 | >67 | >67 |
| | | MX1091 | | | | | | |
| | III | B2 | 0.1268 | 0.7354 | 0.2674 | 1.7245 | 4.3119 | 26.086 |
| | | MX2270 | | | | | | |
| | III | B2_N89Q | 0.7621 | 2.2904 | 1.4345 | 4.703 | 12.9188 | 75.5519 |
| | | MX2271 | | | | | | |

These results showed that B2 swap for E8 does not change the neutralization profile of the antigen binding complexes; glycosylation mutations do not affect the neutralization profile of the antigen binding complexes; and no synergy was observed with tetraspecific antigen binding complexes compared to A18-448 monoclonal antigen binding protein.

### Example 31

### Neutralization Activity of MX2263, MX2264, MX2265, MX2266, and MX2148

The pan-sarbecovirus neutralizing activity of antigen binding complexes MX2263, MX2264, MX2265, MX2266, and MX2148 (FIG. 43, FIG. 69, and FIG. 79) was measured as described above. The results are shown in Tables E45 and E46.

TABLE E45
Pan-sarbecovirus neutralizing activities of MX2263, MX2264, MX2265, MX2266, and
MX2148. IC50 and IC80 are expressed in nM.

| | | | clade 1a | | | | | | | | | |
|---|---|---|---|---|---|---|---|---|---|---|---|---|
| | | | | | Bat CoV | | Bat CoV | | Bat CoV | | Bat CoV | |
| MX | | | SARS-CoV-1 | | WIV1 | | LYRall | | Rs4231 | | RsSHC014 | |
| Specificity | Antibody | Class (RBD) | IC50 | IC80 | IC50 | IC80 | IC50 | IC80 | IC50 | IC80 | IC50 | IC80 |
| Tetraspecific | MX2148 | I + II + | 0.38 | 0.923 | 0.231 | 1.347 | 0.17 | 0.889 | 0.159 | 1.424 | 0.169 | 0.843 |
| | | III + IV | | | | | | | | | | |
| Trispecific | MX2263 | I + III + | 0.047 | 0.198 | 0.088 | 0.601 | 0.07 | 0.266 | 0.078 | 0.786 | 0.204 | 0.472 |
| | | IV | | | | | | | | | | |
| | MX2264 | I + III + | 0.019 | 0.075 | 0.088 | 0.568 | 0.045 | 0.122 | 0.067 | 0.444 | 0.087 | 0.481 |
| | | IV | | | | | | | | | | |
| | MX2265 | I + III + | 0.024 | 0.146 | 0.161 | 0.444 | 0.063 | 0.25 | 0.066 | 0.429 | 0.147 | 0.853 |
| | | IV | | | | | | | | | | |
| | MX2266 | I + III + | 0.025 | 0.104 | 0.048 | 0.423 | 0.034 | 0.228 | 0.07 | 0.558 | 0.244 | 0.675 |
| | | IV | | | | | | | | | | |
| Bispecific | MX2299 | I + IV | 0.18 | 0.598 | 0.089 | 0.445 | 0.081 | 0.246 | 0.07 | 0.675 | 0.065 | 0.476 |
| | MX2300 | I + IV | 0.045 | 0.192 | 0.036 | 0.165 | 0.034 | 0.112 | 0.025 | 0.425 | 0.022 | 0.118 |
| | MX2301 | III + IV | 0.031 | 0.113 | 0.044 | 0.237 | 0.045 | 0.333 | 0.058 | 0.689 | 0.055 | 0.378 |
| | MX2302 | III + IV | 0.057 | 0.152 | 0.047 | 0.273 | 0.039 | 0.083 | 0.026 | 0.352 | 0.052 | 0.17 |
| Monospecific | A18-448 | IV | 0.033 | 0.11 | 0.08 | 0.469 | 0.039 | 0.124 | 0.055 | 0.354 | 0.036 | 0.145 |
| | G11 | II | >67 | >67 | >67 | >67 | N.T. | N.T. | N.T. | N.T. | >67 | >67 |
| | E12 | I | >67 | >67 | >67 | >67 | >67 | >67 | >67 | >67 | >67 | >67 |
| | B2 | III | 0.838 | 2.272 | >67 | >67 | >67 | >67 | >67 | >67 | >67 | >67 |
| | E8 | III | >67 | >67 | >67 | >67 | N.T. | N.T. | N.T. | N.T. | >67 | >67 |

| | | | clade 1b | | | | | |
|---|---|---|---|---|---|---|---|---|
| | | | Pangolin | | Pangolin CoV | | Pangolin CoV | |
| MX | | | CoV GD | | GX-P2V | | GX-P5L | |
| Specificity | Antibody | Class (RBD) | IC50 | IC80 | IC50 | IC80 | IC50 | IC80 |
| Tetraspecific | MX2148 | I + II + | 0.106 | 0.263 | 0.192 | 3.01 | 0.535 | 2.878 |
| | | III + IV | | | | | | |
| Trispecific | MX2263 | I + III + IV | 0.047 | 0.233 | 0.452 | 2.811 | 0.462 | 4.15 |
| | MX2264 | I + III + IV | 0.046 | 0.208 | 3.195 | 39.676 | 1.373 | 27.572 |
| | MX2265 | I + III + IV | 0.064 | 0.216 | 0.284 | 1.783 | 0.132 | 1.658 |
| | MX2266 | I + III + IV | 0.08 | 0.188 | 3.592 | 56.288 | 5.765 | 53.601 |
| Bispecific | MX2299 | I + IV | 0.04 | 0.189 | 0.253 | 1.135 | 0.069 | 0.557 |
| | MX2300 | I + IV | 0.032 | 0.157 | >67 | >67 | >67 | >67 |
| | MX2301 | III + IV | 0.007 | 0.079 | >67 | >67 | >67 | >67 |
| | MX2302 | III + IV | 0.016 | 0.191 | >67 | >67 | >67 | >67 |
| Monospecific | A18-448 | IV | 0.047 | 0.236 | >67 | >67 | >67 | >67 |
| | G11 | II | 0.062 | 0.116 | 13.6 | 47.6 | 17.7 | 49 |
| | E12 | I | 0.134 | 0.763 | 0.05 | 0.354 | 0.036 | 0.353 |
| | B2 | III | 0.079 | 0.466 | >67 | >67 | >67 | >67 |
| | E8 | III | 0.022 | 0.046 | >67 | >67 | >67 | >67 |

TABLE E46
Pan-sarbecovirus neutralizing activities of MX2263, MX2264, MX2265, MX2266, and
MX2148. IC50 and IC80 are expressed in nM.

| | | | SARS-Cov2 | | | | Sars Cov1 clade 1a | | | | | |
|---|---|---|---|---|---|---|---|---|---|---|---|---|
| | | | JN.1 | | HV.1 | | Sars-Cov1 | | Bat CoV WIV1 | | Bat CoV LYR all | |
| Specificity | RBD Class | Antibody | IC50 | IC80 | IC50 | IC80 | IC50 | IC80 | IC50 | IC80 | IC50 | IC80 |
| Trispecific | I + III + IV | MX2263 | 0.013 | 0.057 | 0.024 | 0.082 | 0.047 | 0.198 | 0.088 | 0.601 | 0.070 | 0.266 |
| | I + III + IV | MX2264 | 0.016 | 0.055 | 0.025 | 0.075 | 0.019 | 0.075 | 0.088 | 0.568 | 0.045 | 0.122 |
| | I + III + IV | MX2265 | 0.014 | 0.051 | 0.021 | 0.067 | 0.024 | 0.146 | 0.161 | 0.444 | 0.063 | 0.250 |
| | I + III + IV | MX2266 | 0.014 | 0.055 | 0.011 | 0.055 | 0.025 | 0.104 | 0.048 | 0.423 | 0.034 | 0.228 |
| | I + II + III + | MX2148 | 0.066 | 0.313 | 0.012 | 0.183 | 0.732 | NT | NT | NT | NT | NT |
| | IV | | | | | | | | | | | |

| | Sars Cov1 clade 1a | | | | Sars Cov1 clade 1b | | | | | |
|---|---|---|---|---|---|---|---|---|---|---|
| | Bat CoV | | Bat CoV | | Pangolin CoV | | Pangolin CoV | | Pangolin CoV | |
| | Rs-4231 | | RsSHC014 | | GD | | GX-P2V | | GX-P5L | |
| Specificity | IC50 | IC80 | IC50 | IC80 | IC50 | IC80 | IC50 | IC80 | IC50 | IC80 |
| Trispecific | 0.078 | 0.786 | 0.204 | 0.472 | 0.047 | 0.233 | 0.452 | 2.811 | 0.462 | 4.150 |
| | 0.067 | 0.444 | 0.087 | 0.481 | 0.046 | 0.208 | 3.195 | 39.676 | 1.373 | 27.572 |
| | 0.066 | 0.429 | 0.147 | 0.853 | 0.064 | 0.216 | 0.284 | 1.783 | 0.132 | 1.658 |
| | 0.070 | 0.558 | 0.244 | 0.675 | 0.080 | 0.188 | 3.592 | 56.288 | 5.765 | 53.601 |
| Tetraspecific | NT | NT | NT | NT | 0.106 | 0.263 | 0.192 | 3.010 | 0.535 | 2.878 |

These results showed strong neutralization with tri-specifics antigen binding complexes across all clade 1a and 1b Cov1 strains. Further, these results showed that position of E12 affects neutralization in clade 1b strains.

### Example 32

### Full Panel Neutralization Activity of MX2299, MX2301, MX2265, MX2242 and MX2148

The full panel neutralizing activity of monoclonal antigen binding proteins comprising E12, G11, E8, A18-448, B2, and ADG20; bispecific antigen binding complexes MX2299 and MX2301; trispecific antigen binding complex MX2265; and tetraspecific antigen binding complex MX2242 was measured as described above. The results are shown in Table E47.

TABLE E47
Full panel neutralizing activities of E12, G11, E8, A18-448, B2, ADG20, MX2299, MX2301,
MX2265, and MX2242. IC50 and IC80 are expressed in nM.

| nM | | | WA1 | | D614G | | B.1.427 | | B.1.429 | |
|---|---|---|---|---|---|---|---|---|---|---|
| Specificity | Antibody | Class (RBD) | IC50 | IC80 | IC50 | IC80 | IC50 | IC80 | IC50 | IC80 |
| Monoclonal | E12 | I | 0.218 | 0.387 | 0.048 | 0.220 | 0.129 | 0.463 | 0.104 | 0.329 |
| | G11 | II | 0.823 | 2.048 | 0.189 | 0.442 | 0.406 | 0.819 | 0.136 | 0.285 |
| | E8 | III | 0.037 | 0.102 | 0.009 | 0.022 | 0.022 | 0.091 | 0.011 | 0.029 |
| | A18-448 | IV | 0.277 | 0.482 | 0.039 | 0.141 | 0.108 | 0.334 | 0.077 | 0.172 |
| | B2 | III | 0.124 | 0.291 | 0.025 | 0.068 | 0.309 | 0.461 | 0.022 | 0.079 |
| | ADG20 | I/IV | 0.047 | 0.197 | 0.019 | 0.065 | 0.072 | 0.162 | 0.014 | 0.061 |
| Bispecific | MX2299 | I + IV | 0.079 | 0.266 | 0.027 | 0.056 | 0.137 | 0.252 | 0.023 | 0.070 |
| | MX2301 | III + IV | 0.075 | 0.234 | 0.020 | 0.040 | 0.090 | 0.250 | 0.015 | 0.030 |
| Trispecific | MX2265 | I + III + IV | 0.0992 | 0.3042 | 0.038 | 0.102 | 0.096 | 0.244 | 0.057 | 0.105 |
| Tetraspecific | MX2242 | I + II + III + | 0.1720 | 0.6139 | 0.078 | 0.130 | 0.098 | 0.423 | 0.079 | 0.129 |
| | | IV | | | | | | | | |
| nM | | | P.2 | | B.1.351 | | B.1.1.7 | | B.1.617.1 | |
| Specificity | Antibody | Class (RBD) | IC50 | IC80 | IC50 | IC80 | IC50 | IC80 | IC50 | IC80 |
| Monoclonal | E12 | I | 0.124 | 0.392 | 0.021 | 0.102 | 0.071 | 0.285 | 0.093 | 0.347 |
| | G11 | II | 0.658 | 1.998 | 0.160 | 0.711 | 0.252 | 0.644 | 0.092 | 0.423 |
| | E8 | III | 0.009 | 0.036 | 0.009 | 0.024 | 0.016 | 0.055 | 0.009 | 0.044 |
| | A18-448 | IV | 0.120 | 0.346 | 0.038 | 0.145 | 0.108 | 0.351 | 0.070 | 0.302 |
| | B2 | III | 0.041 | 0.113 | 0.014 | 0.037 | 0.387 | 1.188 | 0.040 | 0.111 |
| | ADG20 | I/IV | 0.034 | 0.121 | 0.011 | 0.050 | 0.106 | 0.424 | 0.015 | 0.071 |
| Bispecific | MX2299 | I + IV | 0.028 | 0.134 | 0.009 | 0.019 | 0.215 | 0.895 | 0.021 | 0.122 |
| | MX2301 | III + IV | 0.026 | 0.064 | 0.009 | 0.020 | 0.182 | 0.699 | 0.010 | 0.044 |
| Trispecific | MX2265 | I + III + IV | 0.059 | 0.116 | 0.020 | 0.060 | 0.075 | 0.132 | 0.071 | 0.174 |
| Tetraspecific | MX2242 | I + II + III + | 0.105 | 0.221 | 0.057 | 0.108 | 0.082 | 0.264 | 0.114 | 0.269 |
| | | IV | | | | | | | | |
| nM | | | B.1.617.2 | | P.1 | | B.1.526 | | B.1.525 | |
| Specificity | Antibody | Class (RBD) | IC50 | IC80 | IC50 | IC80 | IC50 | IC80 | IC50 | IC80 |
| Monoclonal | E12 | I | 0.169 | 0.874 | 0.096 | 0.329 | 0.101 | 0.317 | 0.122 | 0.360 |
| | G11 | II | 0.160 | 1.027 | 0.475 | 1.222 | 0.612 | 2.082 | 0.443 | 1.320 |
| | E8 | III | 0.113 | 0.245 | 0.018 | 0.055 | 0.009 | 0.023 | 0.015 | 0.034 |
| | A18-448 | IV | 0.238 | 0.888 | 0.078 | 0.296 | 0.074 | 0.237 | 0.093 | 0.323 |
| | B2 | III | 0.124 | 0.316 | 0.062 | 0.304 | 0.025 | 0.067 | 0.017 | 0.079 |
| | ADG20 | I/IV | 0.110 | 0.118 | 0.021 | 0.117 | 0.019 | 0.081 | 0.019 | 0.101 |
| Bispecific | MX2299 | I + IV | 0.128 | 0.308 | 0.030 | 0.141 | 0.022 | 0.058 | 0.033 | 0.088 |
| | MX2301 | III + IV | 0.091 | 0.274 | 0.039 | 0.115 | 0.014 | 0.030 | 0.014 | 0.043 |
| Trispecific | MX2265 | I + III + IV | 0.244 | 0.454 | 0.066 | 0.112 | 0.055 | 0.102 | 0.070 | 0.117 |
| Tetraspecific | MX2242 | I + II + III + | 0.331 | 0.509 | 0.110 | 0.208 | 0.075 | 0.126 | 0.090 | 0.141 |
| | | IV | | | | | | | | |
| nM | | | B.1.621 | | BA.1 | | BA.4/5 | | CH.1.1 | |
| Specificity | Antibody | Class (RBD) | IC50 | IC80 | IC50 | IC80 | IC50 | IC80 | IC50 | IC80 |
| Monoclonal | E12 | I | 0.106 | 0.348 | 0.013 | 0.046 | 0.022 | 0.059 | 0.064 | 0.276 |
| | G11 | II | 0.578 | 2.113 | 0.372 | 2.269 | 0.197 | 0.758 | 43.132 | >67 |
| | E8 | III | 0.016 | 0.055 | 0.074 | 0.836 | 0.005 | 0.014 | 40.527 | >67 |
| | A18-448 | IV | 0.152 | 0.390 | 0.035 | 0.128 | 0.017 | 0.050 | 0.051 | 0.185 |
| | B2 | III | 0.120 | 0.363 | 0.018 | 0.087 | 0.039 | 0.115 | 3.776 | 10.493 |
| | ADG20 | I/IV | 0.026 | 0.131 | 13.904 | >67 | >67 | >67 | 13.726 | >67 |
| Bispecific | MX2299 | I + IV | 0.062 | 0.238 | 0.006 | 0.018 | 0.007 | 0.019 | 0.025 | 0.082 |
| | MX2301 | III + IV | 0.032 | 0.119 | 0.010 | 0.024 | 0.011 | 0.026 | 0.091 | 0.320 |
| Trispecific | MX2265 | I + III + IV | 0.092 | 0.176 | 0.006 | 0.023 | 0.004 | 0.017 | 0.022 | 0.059 |
| Tetraspecific | MX2242 | I + II + III + | 0.159 | 0.241 | 0.009 | 0.043 | 0.003 | 0.022 | 0.048 | 0.100 |
| | | IV | | | | | | | | |
| nM | | | XBB | | XBB.1.5 | | XBB.2.3.2 | | XBB.1.16.6 | |
| Specificity | Antibody | Class (RBD) | IC50 | IC80 | IC50 | IC80 | IC50 | IC80 | IC50 | IC80 |
| Monoclonal | E12 | I | 0.048 | 0.196 | 0.037 | 0.126 | 0.062 | 0.431 | 0.972 | 7.134 |
| | G11 | II | 0.273 | 1.826 | 0.282 | 2.266 | 0.853 | 5.064 | 0.808 | 4.174 |
| | E8 | III | 4.775 | 65.598 | 23.740 | >67 | 25.637 | >67 | >67 | >67 |
| | A18-448 | IV | 0.017 | 0.065 | 0.021 | 0.084 | 0.045 | 0.138 | 0.016 | 0.082 |
| | B2 | III | 0.256 | 0.787 | 0.114 | 0.823 | 0.457 | 2.898 | 0.039 | 0.682 |
| | ADG20 | I/IV | 10.623 | >67 | >67 | >67 | >67 | >67 | >67 | >67 |
| Bispecific | MX2299 | I + IV | 0.009 | 0.034 | 0.008 | 0.030 | 0.014 | 0.051 | 0.021 | 0.077 |
| | MX2301 | III + IV | 0.022 | 0.092 | 0.022 | 0.093 | 0.034 | 0.179 | 0.025 | 0.107 |
| Trispecific | MX2265 | I + III + IV | 0.018 | 0.055 | 0.008 | 0.031 | 0.037 | 0.079 | 0.031 | 0.078 |
| Tetraspecific | MX2242 | I + II + III + | 0.024 | 0.052 | 0.020 | 0.058 | 0.051 | 0.088 | 0.059 | 0.146 |
| | | IV | | | | | | | | |
| nM | | | EG.5.1 | | BA.2.86 | | JD.1.1 | | JN.1 | |
| Specificity | Antibody | Class (RBD) | IC50 | IC80 | IC50 | IC80 | IC50 | IC80 | IC50 | IC80 |
| Monoclonal | E12 | I | 0.859 | 5.554 | 0.028 | 0.107 | 20.268 | >67 | 2.640 | 14.089 |
| | G11 | II | 0.394 | 2.259 | >67 | >67 | 1.040 | 5.364 | >67 | >67 |
| | E8 | III | 35.215 | >67 | >67 | >67 | 23.098 | >67 | >67 | >67 |
| | A18-448 | IV | 0.021 | 0.074 | 0.033 | 0.109 | 0.034 | 0.093 | 0.039 | 0.117 |
| | B2 | III | 0.048 | 0.769 | 0.058 | 0.389 | 0.197 | 0.984 | 0.097 | 0.451 |
| | ADG20 | I/IV | >67 | >67 | >67 | >67 | >67 | >67 | >67 | >67 |
| Bispecific | MX2299 | I + IV | 0.016 | 0.060 | 0.009 | 0.028 | 0.035 | 0.140 | 0.025 | 0.112 |
| | MX2301 | III + IV | 0.021 | 0.088 | 0.016 | 0.059 | 0.031 | 0.113 | 0.023 | 0.086 |
| Trispecific | MX2265 | I + III + IV | 0.007 | 0.040 | 0.011 | 0.045 | 0.017 | 0.080 | 0.014 | 0.051 |
| TetraspecifiC | MX2242 | I + II + III + | 0.011 | 0.045 | 0.039 | 0.159 | 0.027 | 0.062 | 0.100 | 0.226 |
| | | IV | | | | | | | | |
| nM | | | JN.1.13.1 | | KP.2 | | KP.2.3 | | LB.1 | |
| Specificity | Antibody | Class (RBD) | IC50 | IC80 | IC50 | IC80 | IC50 | IC80 | IC50 | IC80 |
| Monoclonal | E12 | I | 4.990 | 39.815 | 31.271 | >67 | >67 | >67 | 59.314 | >67 |
| | G11 | II | >67 | >67 | >67 | >67 | >67 | >67 | >67 | >67 |
| | E8 | III | >67 | >67 | >67 | >67 | >67 | >67 | >67 | >67 |
| | A18-448 | IV | 0.041 | 0.114 | 0.023 | 0.084 | 0.076 | 0.167 | 0.038 | 0.116 |
| | B2 | III | 0.502 | 2.798 | 0.393 | 1.516 | 1.517 | 5.755 | 0.684 | 4.515 |
| | ADG20 | I/IV | >67 | >67 | >67 | >67 | >67 | >67 | >67 | >67 |
| Bispecific | MX2299 | I + IV | 0.027 | 0.116 | 0.048 | 0.187 | | | | |
| | MX2301 | III + IV | 0.047 | 0.185 | 0.033 | 0.106 | 0.072 | 0.205 | 0.064 | 0.196 |
| Trispecific | MX2265 | I + III + IV | 0.050 | 0.107 | 0.032 | 0.112 | 0.043 | 0.119 | 0.049 | 0.113 |
| Tetraspecific | MX2242 | I + II + III + | 0.082 | 0.192 | 0.047 | 0.181 | 0.136 | 0.410 | 0.059 | 0.255 |
| | | IV | | | | | | | | |

| nM | | | KP.3 | | KP.3.1.1 | |
|---|---|---|---|---|---|---|
| Specificity | Antibody | Class (RBD) | IC50 | IC80 | IC50 | IC80 |
| Monoclonal | E12 | I | >67 | >67 | >67 | >67 |
| | G11 | II | >67 | >67 | >67 | >67 |
| | E8 | III | >67 | >67 | >67 | >67 |
| | A18-448 | IV | 0.016 | 0.055 | 0.046 | 0.140 |
| | B2 | III | 0.083 | 0.399 | 0.207 | 0.820 |
| | ADG20 | I/IV | >67 | >67 | >67 | >67 |
| Bispecific | MX2299 | I + IV | 0.054 | 0.177 | | |
| | MX2301 | III + IV | 0.023 | 0.070 | 0.045 | 0.118 |
| Trispecific | MX2265 | I + III + IV | 0.016 | 0.060 | 0.049 | 0.115 |
| Tetraspecific | MX2242 | I + II + III + | 0.041 | 0.126 | 0.086 | 0.254 |
| | | IV | | | | |

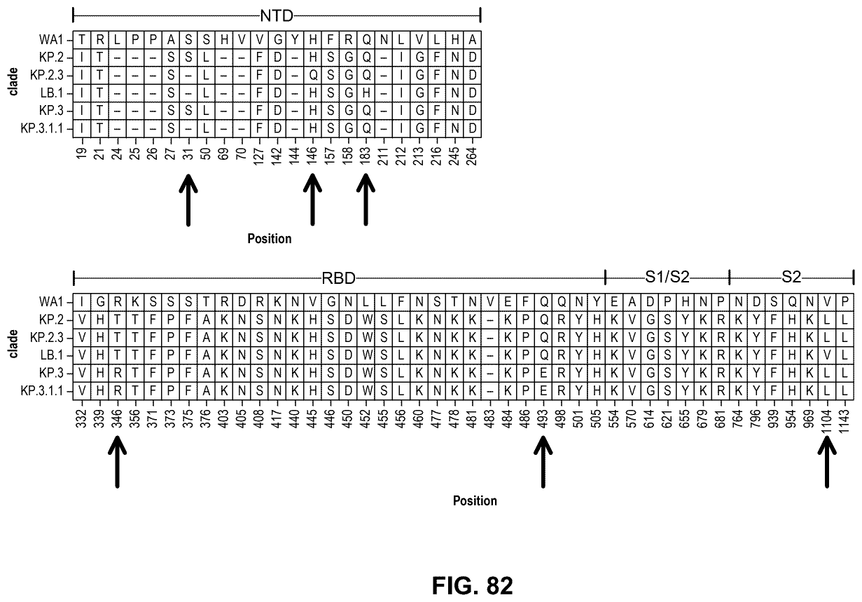

Further, as shown in Table E48, the multispecific antibodies remain potent against current variants. The spike amino acid differences in KP.2, KP.2.3, LB.1, KP.3, and KP.3.1.1, with respect to WA1, are shown in FIG. 82.

TABLE E48
Neutralizing activities of E12, G11, E8, A18-448, B2, ADG20, MX2301, MX2265, and
MX2242. IC50 and IC80 are expressed in nM.

| nM | | | KP.2 | | KP.2.3 | | LB.1 | | KP.3 | | KP.3.1.1 | |
|---|---|---|---|---|---|---|---|---|---|---|---|---|
| Specificity | Antibody | Class (RBD) | IC50 | IC80 | IC50 | IC80 | IC50 | IC80 | IC50 | IC80 | IC50 | IC80 |
| Monoclonal | E12 | I | 31.271 | >67 | >67 | >67 | 59.314 | >67 | >67 | >67 | >67 | >67 |
| | G11 | II | >67 | >67 | >67 | >67 | >67 | >67 | >67 | >67 | >67 | >67 |
| | E8 | III | >67 | >67 | >67 | >67 | >67 | >67 | >67 | >67 | >67 | >67 |
| | A18-448 | IV | 0.023 | 0.084 | 0.076 | 0.167 | 0.038 | 0.116 | 0.016 | 0.055 | 0.046 | 0.140 |
| | B2 | III | 0.393 | 1.516 | 1.517 | 5.755 | 0.684 | 4.515 | 0.083 | 0.399 | 0.207 | 0.820 |
| | ADG20 | I/IV | >67 | >67 | >67 | >67 | >67 | >67 | >67 | >67 | >67 | >67 |
| Bispecific | MX2301 | III + IV | 0.033 | 0.106 | 0.072 | 0.205 | 0.064 | 0.196 | 0.023 | 0.070 | 0.045 | 0.118 |
| Trispecific | MX2265 | I + III + IV | 0.032 | 0.112 | 0.043 | 0.119 | 0.049 | 0.113 | 0.016 | 0.060 | 0.049 | 0.115 |
| Tetraspecific | MX2242 | I + II + III + | 0.047 | 0.181 | 0.136 | 0.410 | 0.059 | 0.255 | 0.041 | 0.126 | 0.086 | 0.254 |
| | | IV | | | | | | | | | | |

### Example 33

### Live Virus Neutralization of MX2148

A Plaque Reduction Neutralization Test (PRNT) was perform to assay the live virus neutralization activity of MX2148, VERO E6 cells were used to test the live virus neutralization activity of MX2148 against WA-1, and Vero-TMPRSS2-ACE2 cells were used to test the live virus neutralization activity of MX2128 against B.1.1.7, B.1.351, B.1.617.2, BA.1.1.529, BA.2.86, BA.5, and JN.1. The live virus neutralization activity of MX2148 was compared with the live virus neutralization activity of E12, G11, E8, and A18-448 (Table E49). Antibody dilution series and viruses were added to target cells, and the IC50 was calculated from the PFU/ml and reported at PRNT/ml (μg/ml), MX2148_showed a PRNT50 of <0.206 against WA-1, B.1.1.7, B.1.351, B.1.617.2, BA.1.1.529, BA.2.86, and BA.5; and of 0.281 against JN.1. Each of the comparative monoclonals E12, G11, E8 and 448 had the results shown in Table E49 against the respective variants.

TABLE E49
Live virus neutralization activity of MX2148. (PRNT50 is
expressed in mg/ml; MX2148: 1 mg/ml = 5 nM).

| | | PRNT50 (mg/ml) | | | | |
|---|---|---|---|---|---|---|
| WHO | Variant | MX2148 | E12 | G11 | E8 | 448 |
| | WA-1 | <0.206 | <0.206 | <0.206 | <0.206 | <0.206 |
| Alpha | B.1.1.7 | <0.206 | <0.206 | <0.206 | <0.206 | <0.206 |
| Beta | B.1.351 | <0.206 | <0.206 | 0.480 | <0.206 | <0.206 |
| Delta | B.1.617.2 | <0.206 | <0.206 | <0.206 | <0.206 | <0.206 |
| Omicron | BA.1.1.529 | <0.206 | <0.206 | 1.944 | <0.206 | <0.206 |
| Omicron | BA.2.86 | <0.206 | <0.206 | >50 | >50 | <0.206 |
| Omicron | BA.5 | <0.206 | <0.206 | 0.446 | <0.206 | <0.206 |
| Omicron | JN.1 | 0.281 | 3.854 | >50 | >50 | <0.206 |

### Example 34

### Pharmacokinetics (PK) of MX2567, MX2301, MX2242, and MX2265 in Humanized FcRn Mice

Human FcRn transgenic mice (C57BL/6, B6.mFcRn^{-/-} hFcRn Tg32 line from The Jackson Laboratory) were used to assess the pharmacokinetics of MX2567, MX2301, MX2242, and MX2265. Each animal was infused intravenously with 5 mg antibody/kg of body weight. Whole blood samples were collected at day 1, 2, 5, 7, 9, 14, 21. Serum was separated by centrifugation. Serum antibody levels were measured by ELISA. The antigen binding polypeptide complexes used in this experiment were produced in CHOK1 cells. All the tested antigen binding polypeptide complexes comprise a TM/YTE Fc modification, except MX2567, which comprises an LS modification.

All mice were bred and maintained under pathogen-free conditions at an American Association for Assessment and Accreditation of Laboratory Animal Care International-accredited animal facility at the National Institute of Allergy and Infectious Diseases and housed in accordance with the procedures outlined in the Guide for the Care and Use of Laboratory Animals. All mice were between 6 and 13 weeks of age.

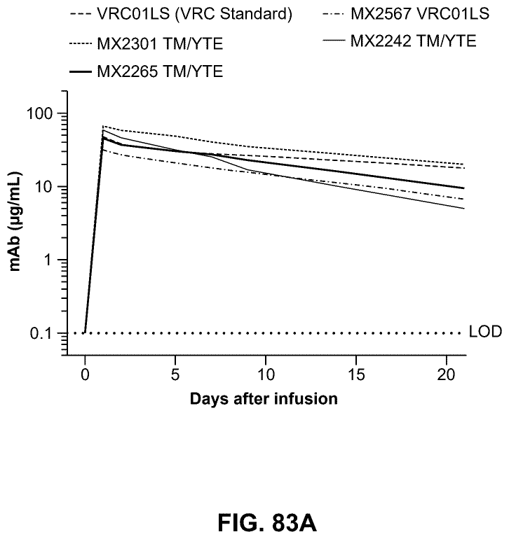

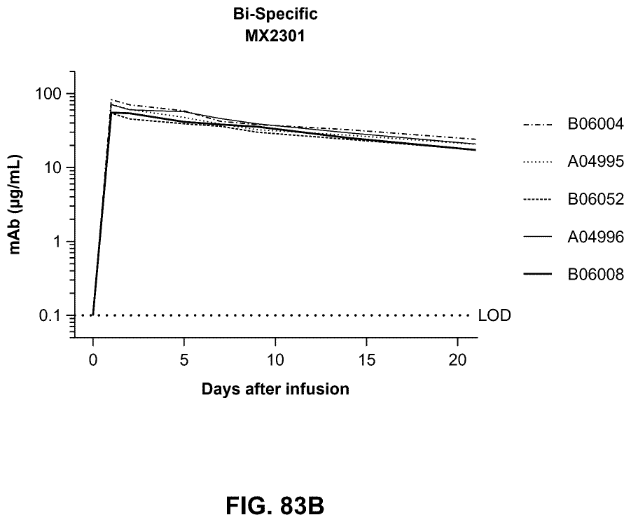

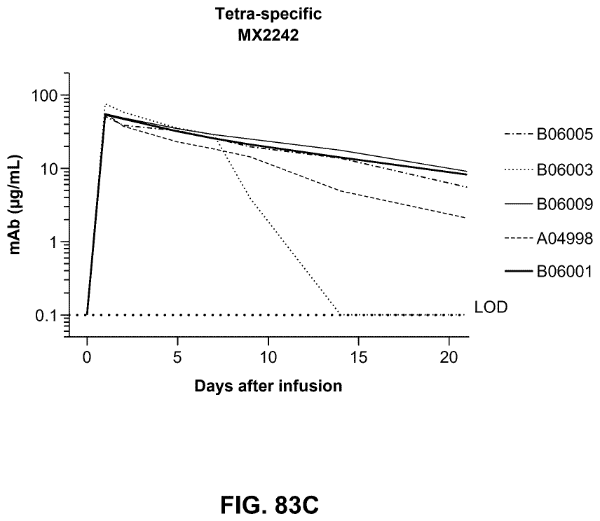

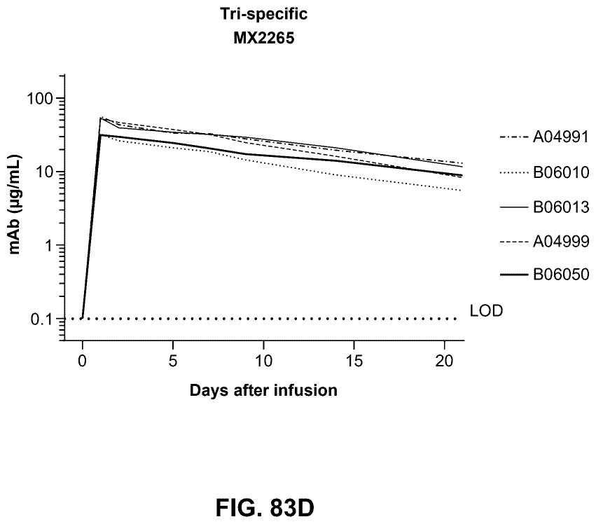

The PK (day 0-20) was determined for each of the tested antigen binding polypeptide complexes, and plotted as shown in FIG. 83A-83D.

### Example 35

### Generation of MX2676, MX2675, MX2674, MX2673, MX2672, MX2671, and MX2670

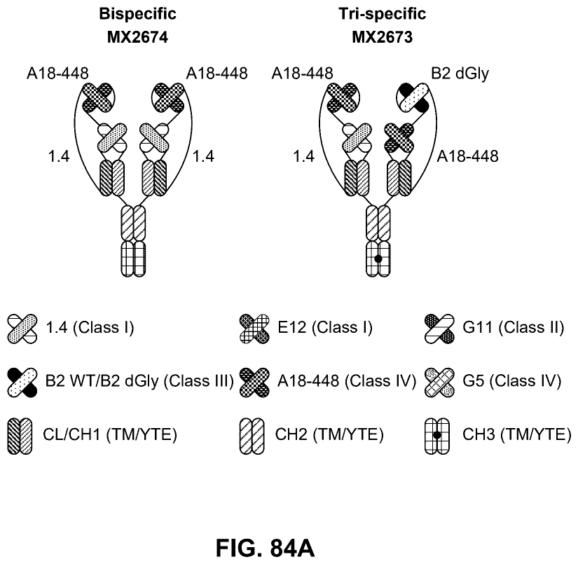

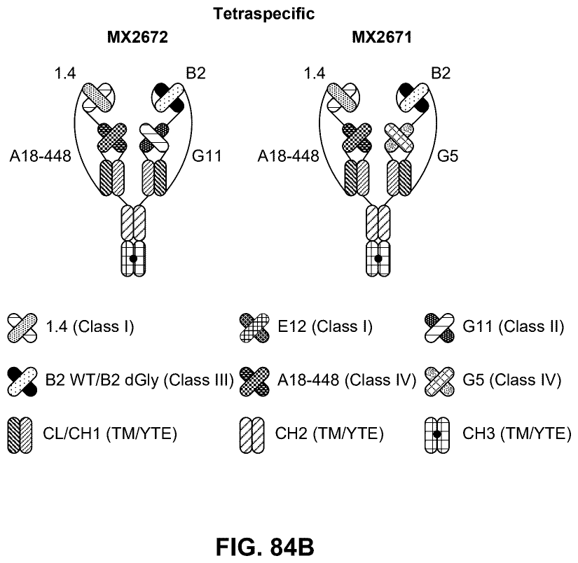

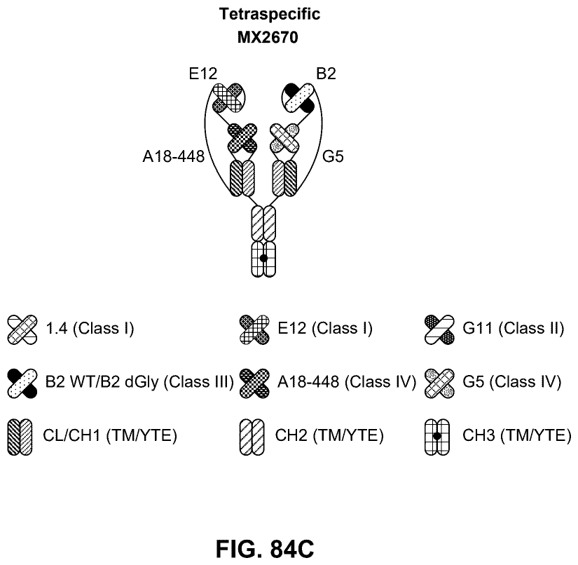

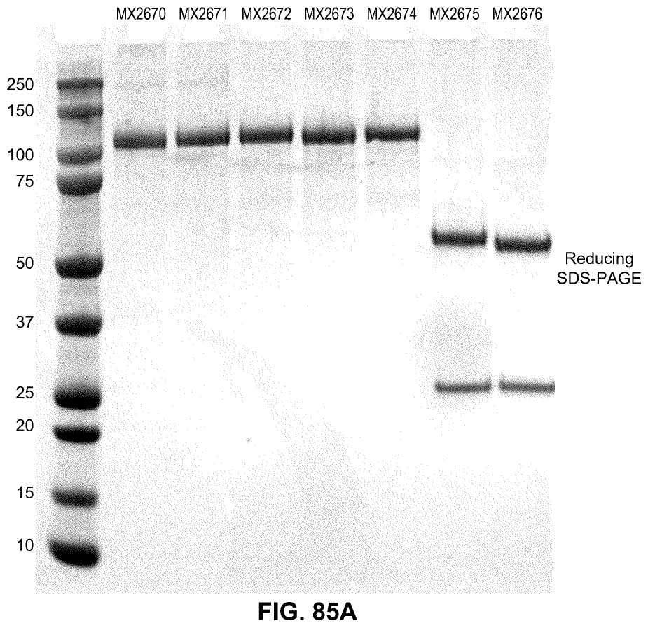

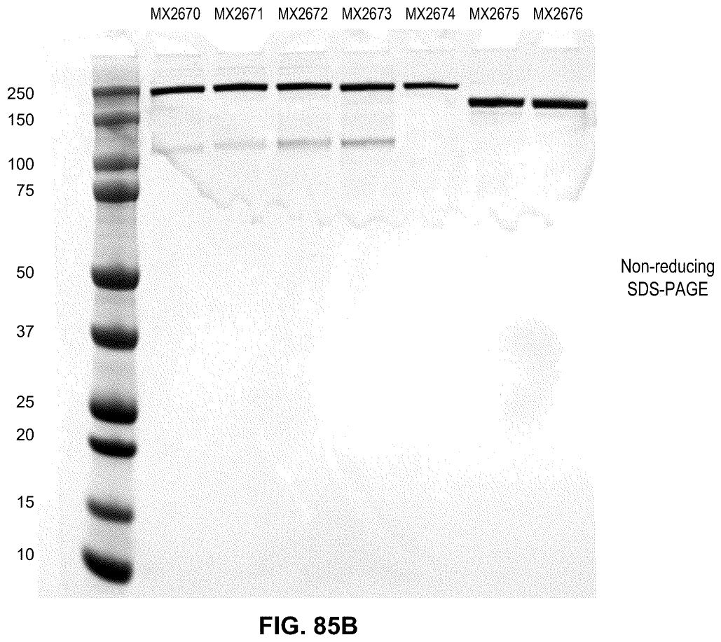

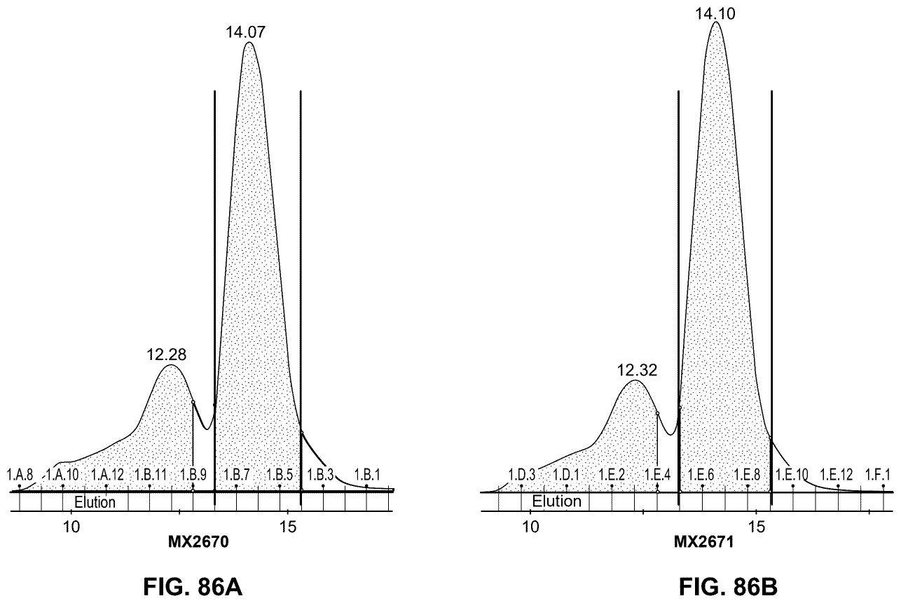

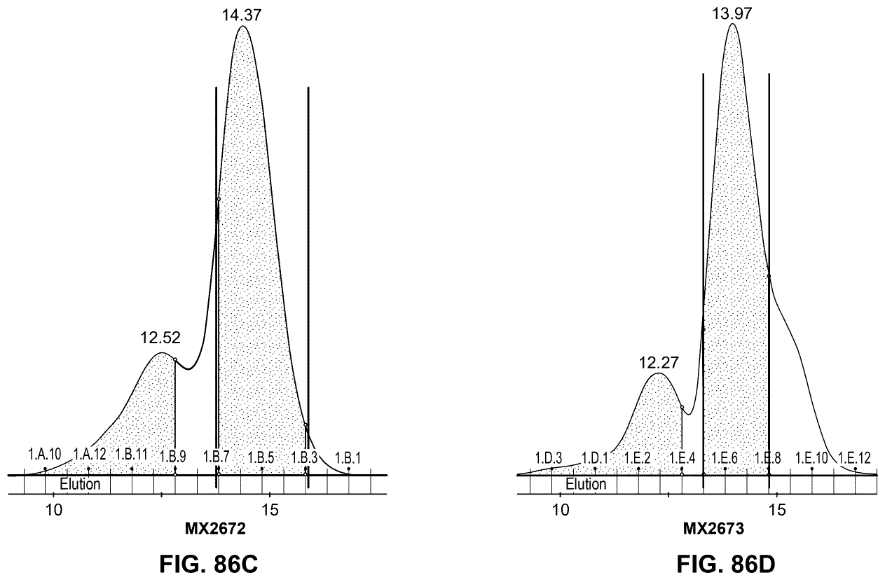

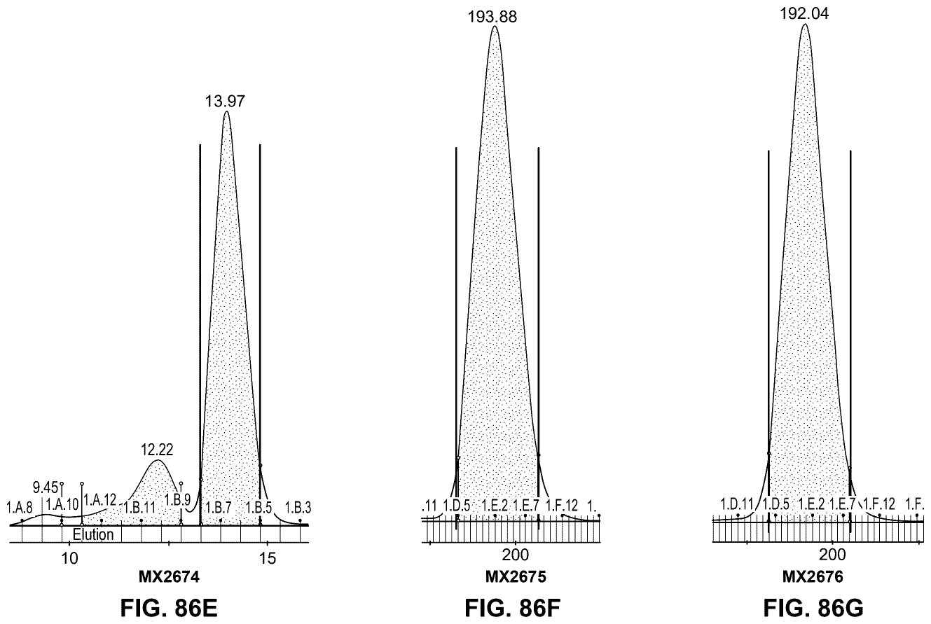

MX2674 (bispecific), MX2673 (trispecific), MX2672, MX2671, and MX2670 (tetraspecific) (FIGS. 84A-84C), antigen binding complexes and MX2676 and MX2675 (monoclonal antibodies of A63-1.4 and G5) were produced and analyzed on reduced (FIG. 85A) and non-reduced (FIG. 85B) SDS-PAGE, SEC purification was performed and the results are shown in FIGS. 86A-86G.

### Example 36

### Comparison of Monospecific Monovalent, Bivalent and Tetravalent Antigen Binding Peptides/Complexes

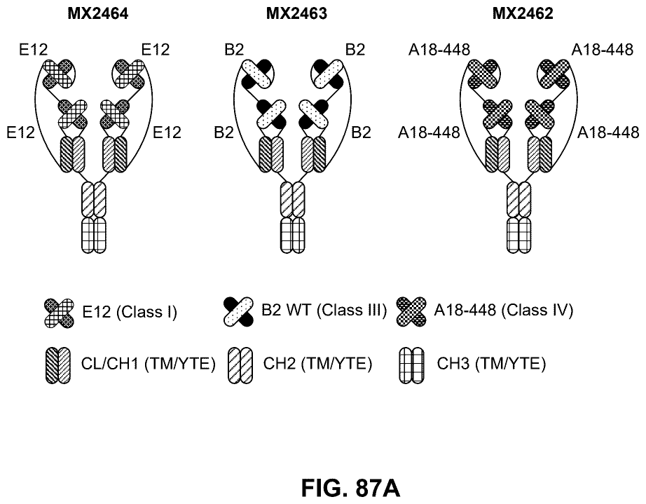

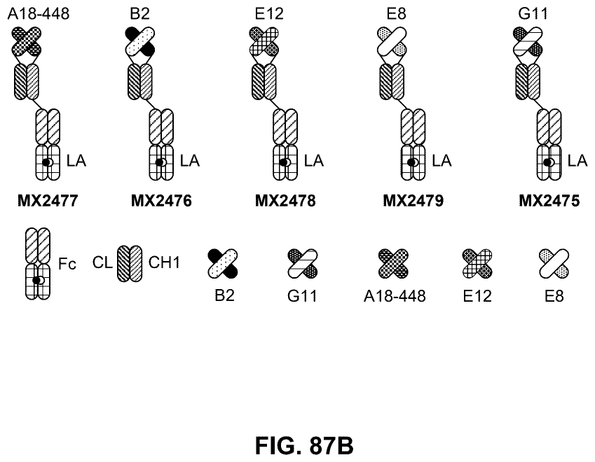

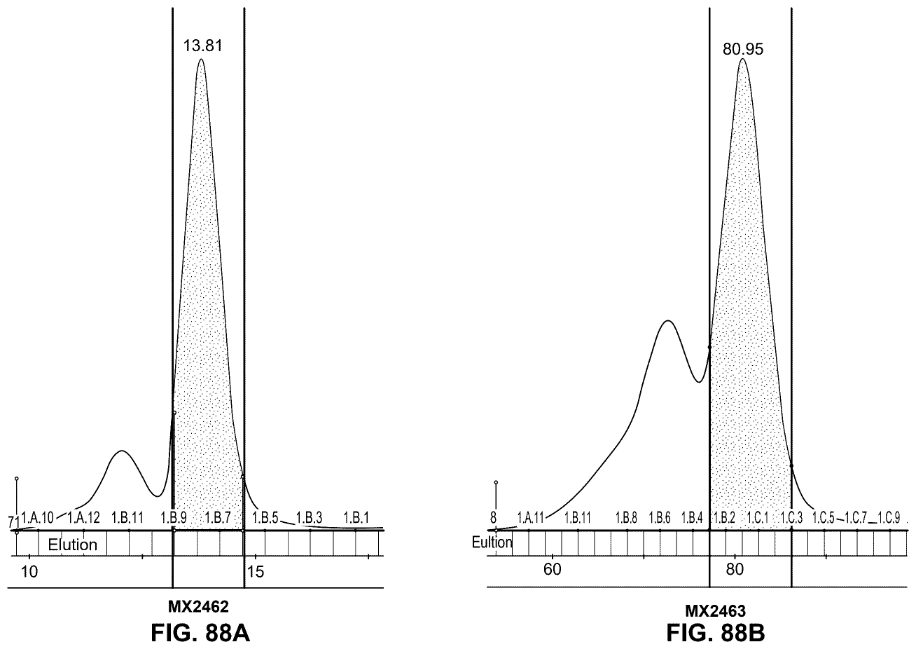

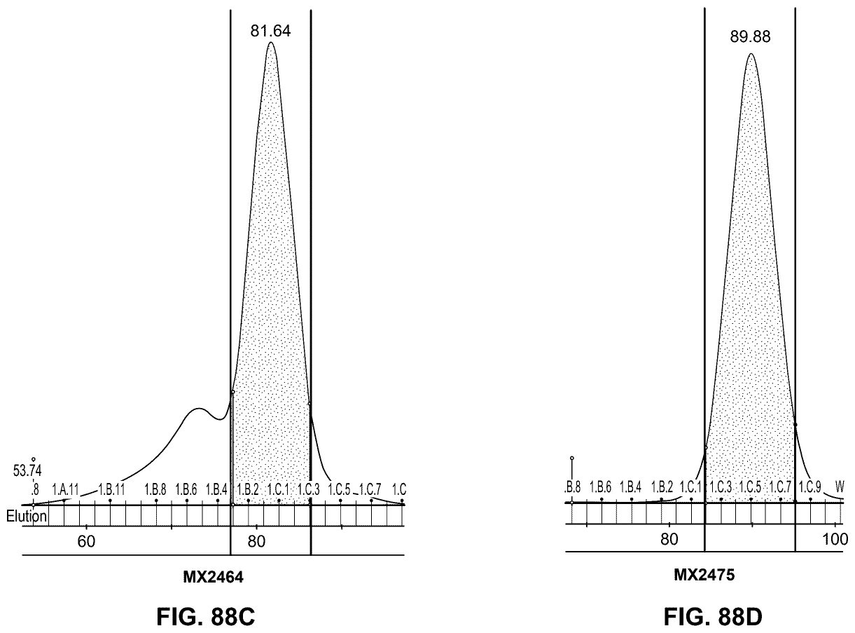

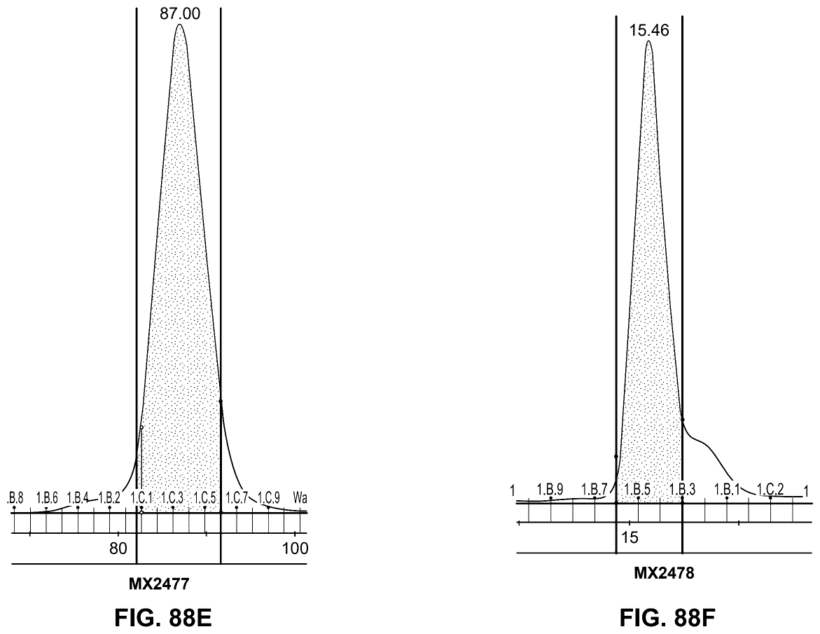

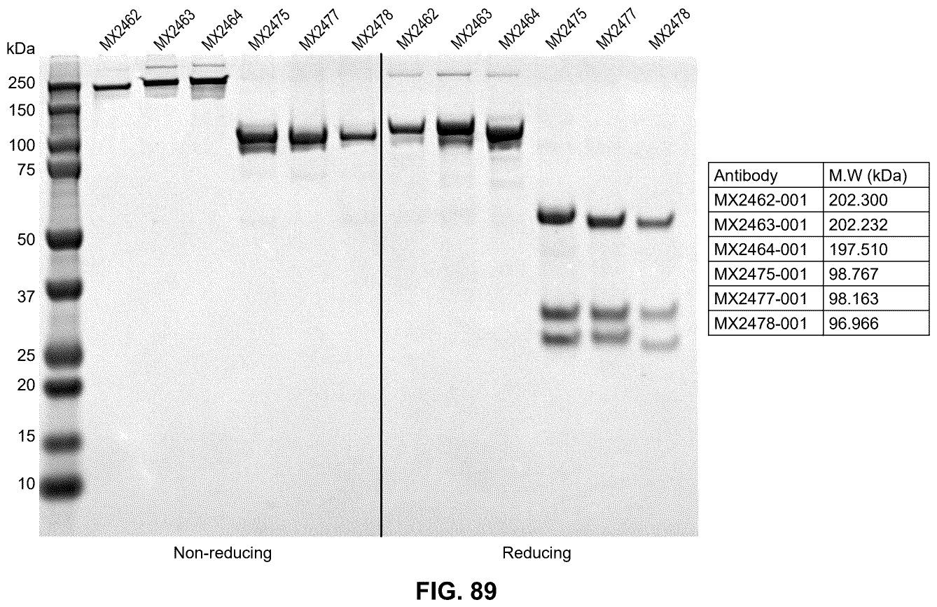

MX2464, MX2463, and MX2462 (tetravalent monospecific) (FIG. 87A); and MX2477, MX2476, MX2478, MX2479, and MX2475 (monovalent monospecific) (FIG. 87B) antigen binding complexes were produced. Protein A, and SEC purification (MES (2-(N-morpholino) ethanesulfonic acid) buffering agent) of MX2464, MX2463, MX2462, MX2475, MX2477 and MX2478 was performed and the results are shown in FIGS. 88A-88F, and in Table 50, MX2464, MX2463, MX2462, MX2475, MX2477 and MX2478 were analyzed on reduced and non-reduced SDS-PAGE (FIG. 89).

TABLE E50
Production of MX2464, MX2463, MX2462, MX2475, MX2477
and MX2478 in ExpiCHO cells.

| | | | mg/L | mg/L | mg/L | mg |
|---|---|---|---|---|---|---|
| MX | | | Protein A | After MES | After SEC | Total |
| number | Note | Cell line | yield | yield | yield | purified |
| MX2462 | 448 tetravalent | ExpiCHO | 1.9 | 1.9 | 0.71 | 0.32 |
| | monospecific | | | | | |
| MX2463 | B2 tetravalent | ExpiCHO | 6.6 | 6.6 | 2.89 | 1.3 |
| | monospecific | | | | | |
| MX2464 | E12 tetravalent | ExpiCHO | 11.4 | 11.4 | 6 | 2.7 |
| | monospecific | | | | | |
| MX2475 | G11 monovalent | ExpiCHO | 42.4 | 26.0 | 19.0 | 4.752 |
| MX2477 | 448 monovalent | ExpiCHO | 28.3 | 22.4 | 20.2 | 10.08 |
| MX2478 | E12 monovalent | ExpiCHO | 2.6 | N.D | 0.51 | 0.255 |

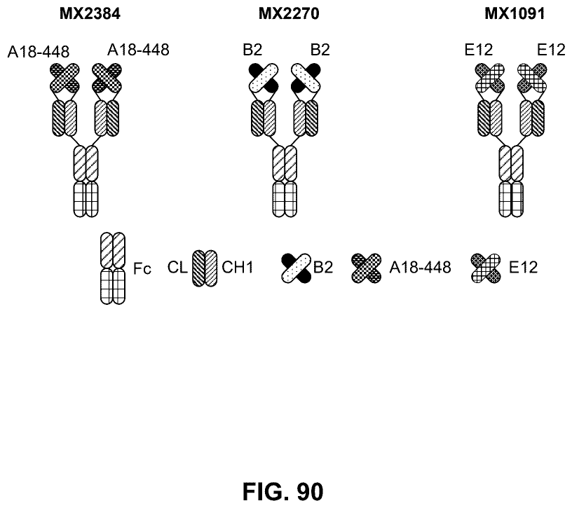

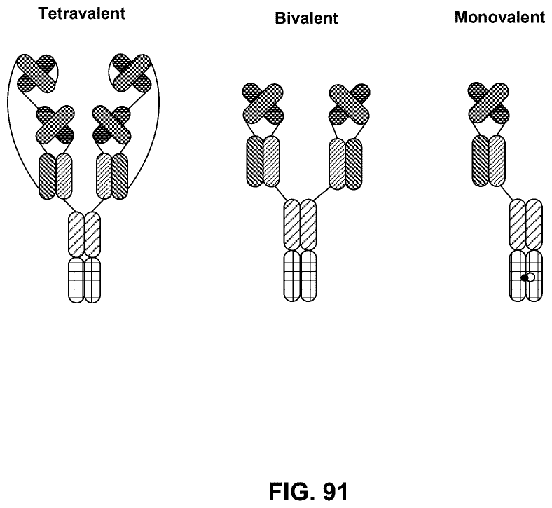

MX2384, MX2270, and MX1091 (FIG. 90) were also produced, to analyze the neutralization power of monospecific monovalent, bivalent and tetravalent. The neutralization power of monospecific monovalent, bivalent and tetravalent antigen binding peptides/complexes (FIG. 91) was measured and compared to the neutralization power of tetravalent and tetraspecific MX2148. The results are shown in Table 51.

TABLE E51
Neutralizing activities monospecific monovalent, bivalent and tetravalent antigen binding
peptides/complexes and of MX2148. IC50 and IC80 are expressed in nM.

| | | EG.5.1 | | JD.1.1 | | JN.1 | | KP.2 | | |
|---|---|---|---|---|---|---|---|---|---|---|
| Antibody | Class (RBD) | IC50 | IC80 | IC50 | IC80 | IC50 | IC80 | IC50 | IC80 | |
| 448 monovalent | IV | 0.637 | 4.406 | 1.390 | 7.198 | 0.548 | 5.165 | 0.518 | 5.005 | |
| 448 bivalent | IV | 0.017 | 0.081 | 0.025 | 0.088 | 0.026 | 0.093 | 0.025 | 0.085 | |
| 448 tetravalent | IV | 0.006 | 0.023 | 0.007 | 0.022 | 0.008 | 0.026 | 0.007 | 0.032 | |
| | | EG.5.1 | | JD.1.1 | | JN.1 | | KP.2 | | |
| Antibody | Class (RBD) | IC50 | IC80 | IC50 | IC80 | IC50 | IC80 | IC50 | IC80 | |
| B2 monovalent | III | 1.205 | 39.533 | 37.029 | >67 | 1.455 | 21.887 | 27.375 | >67 | |
| B2 bivalent | III | 0.144 | 0.771 | 0.466 | 1.413 | 0.105 | 0.524 | 0.564 | 2.490 | |
| B2 tetravalent | III | 0.043 | 0.273 | 0.083 | 0.380 | 0.030 | 0.178 | 0.103 | 0.697 | |
| | | EG.5.1 | | JD.1.1 | | JN.1 | | KP.2 | | |
| Antibody | Class (RBD) | IC50 | IC80 | IC50 | IC80 | IC50 | IC80 | IC50 | IC80 | |
| E12 monovalent | I | 2.677 | 37.186 | >67 | >67 | 2.338 | >67 | 55.792 | >67 | |
| E12 bivalent | I | 1.264 | 5.042 | 31.091 | >67 | 1.195 | 13.160 | 25.817 | >67 | |
| E12 tetravalent | I | 0.070 | 0.561 | 6.501 | 24.202 | 0.083 | 0.914 | 4.797 | 12.504 | |
| | | | EG.5.1 | | JD.1.1 | | JN.1 | | KP.2 | |
| Specificity | Antibody | Class (RBD) | IC50 | IC80 | IC50 | IC80 | IC50 | IC80 | IC50 | IC80 |
| Tetraspecific | MX2148 | I + II + III + | 0.018 | 0.064 | 0.035 | 0.092 | 0.045 | 0.258 | 0.103 | 0.377 |
| | | IV | | | | | | | | |

### Example 37

### Comparison of Antigen Binding Peptides/Complexes

Neutralizing activities of MX2148, MX2301, and MX2673 were measured and compared to the neutralizing activity of AZD3152; (AstraZeneca) Phase III efficacy, ADG20; (Adagio) Phase III efficacy, and VYD222; (Invivyd); U.S. EUA. The neutralization activity IC50 (μg/ml) is shown in Table E52.

TABLE E52
Neutralizing activities of MX2148, MX2301, MX2673, AZD3152, ADG20, and VYD222 (IC50 (μg/ml)).

| Virus | AZD3152 | ADG20 | VYD222 | MX2148 | MX2301 | MX2673 |
|---|---|---|---|---|---|---|
| D614G | 0.015 | 0.003 | 0.010 | 0.013 | 0.004 | 0.007 |
| CH.1.1 | 0.045 | 2.058 | 1.489 | 0.014 | 0.018 | 0.005 |
| EG.5.1 | >10 | >10 | 0.899 | 0.011 | 0.004 | 0.001 |
| BA.2.86 | 0.053 | >10 | 2.127 | 0.024 | 0.003 | 0.002 |
| JN.1 | 0.766 | >10 | 0.706 | 0.102 | 0.005 | 0.001 |
| KP.2 | >10 | >10 | 0.684 | 0.215 | 0.007 | 0.003 |
| KP.2.3 | >10 | >10 | 4.250 | 1.074 | 0.014 | 0.006 |
| LB.1 | >10 | >10 | 4.615 | 0.765 | 0.013 | 0.005 |
| KP3 | >10 | >10 | 1.324 | 0.609 | 0.005 | 0.001 |
| KP.3.1.1 | >10 | >10 | 4.054 | 0.741 | 0.009 | 0.003 |
| XEC | >10 | >10 | >10 | 0.695 | 0.006 | 0.002 |

The neutralizing activities of MX2301 (bispecific), MX2148 (tetraspecific), MX2673 (trispecific), and MX2674 (bispecific) were further evaluated and compared to the neutralizing activity of A63-1.4, E12, E8, G11, A18-448, B2, AZD3152, ADG20, BD55-1205, and VYD222 (monoclonal antibodies). The neutralization activity IC50 and IC80 (μg/ml) is shown in Table E53.

TABLE E53
Neutralizing activities of MX230, MX2148, MX2673, and MX2674, A63-1.4, E12, E8, G11,
A18-448, B2, AZD3152, ADG20, BD55-1205, and VYD2222. (IC50 and IC80 (μg/ml)).

| | Anti- | Speci- | Class | D614G | | CH.1.1 | | EG.5.1 | | BA.2.86 | | JN.1 | | KP.2 | |
|---|---|---|---|---|---|---|---|---|---|---|---|---|---|---|---|
| | body | ficity | (RBD) | IC50 | IC80 | IC50 | IC80 | IC50 | IC80 | IC50 | IC80 | IC50 | IC80 | IC50 | IC80 |
| Our | A63-1.4 | Mono- | I | | | | | | | | | | | | |
| bind- | | clonal | | | | | | | | | | | | | |
| ers | E12 | Mono- | I | | | | | | 5.554 | | | 2.640 | 14.089 | 31.271 | >67 |
| | | clonal | | | | | | | | | | | | | |
| | E8 | Mono- | II | | | 40.527 | >67 | 35.215 | >67 | >67 | >67 | >67 | >67 | >67 | >67 |
| | | clonal | | | | | | | | | | | | | |
| | G1 | Mono- | III | | | 43.132 | >67 | | 2.259 | >67 | >67 | >67 | >67 | >67 | >67 |
| | | clonal | | | | | | | | | | | | | |
| | A18-448 | Mono- | IV | | | | | | | | | | | | |
| | | clonal | | | | | | | | | | | | | |
| | B2 | Mono- | III | | | 3.776 | 10.493 | | | | | | | | 1.516 |
| | | clonal | | | | | | | | | | | | | |
| Com- | AZD3 | Mono- | I | | | | 1.594 | >67 | >67 | | 2.228 | 5.108 | 27.955 | >67 | >67 |
| peti- | 152 | clonal | | | | | | | | | | | | | |
| tion | ADG20 | Mono- | I/IV | | | 13.726 | >67 | >67 | >67 | >67 | >67 | >67 | >67 | >67 | >67 |
| | | clonal | | | | | | | | | | | | | |
| | BD55- | Mono- | I | | | | | | | | | | | | |
| | 1205 | clonal | | | | | | | | | | | | | |
| | VYD222 | Mono- | I/IV | | | 9.932 | 30.414 | 5.999 | 20.875 | 14.185 | 36.733 | 4.709 | 15.557 | 4.564 | 16.546 |
| | | clonal | | | | | | | | | | | | | |
| Our | MX2301 | Bi- | III + | | | | | | | | | | | | |
| mole- | | speci- | IV | | | | | | | | | | | | |
| cules | | fic | | | | | | | | | | | | | |
| | MX2148- | Tetra- | I + | | | | | | | | | | 1.525 | 1.076 | 3.723 |
| | 006 | speci- | II + | | | | | | | | | | | | |
| | | fic | III + | | | | | | | | | | | | |
| | | | IV | | | | | | | | | | | | |
| | MX2673 | Tri- | I + | | | | | | | | | | | | |
| | | speci- | III + | | | | | | | | | | | | |
| | | fic | IV | | | | | | | | | | | | |
| | MX2674 | Bi- | I + | | | | | | | | | | | | |
| | | speci- | IV | | | | | | | | | | | | |
| | | fic | | | | | | | | | | | | | |

Neutralizing activities of MX230, MX2148, MX2673, and MX2674, A63-1.4, E12, E8, G11,
A18-448, B2, AZD3152, ADG20, BD55-1205, and VYD2222. (IC50 and IC80 (μg/ml)).

| | Anti- | Speci- | Class | KP.2.3 | | LB.1 | | KP.3 | | KP.3.1.1 | | XEC | |
|---|---|---|---|---|---|---|---|---|---|---|---|---|---|
| | body | ficity | (RBD) | IC50 | IC80 | IC50 | IC80 | IC50 | IC80 | IC50 | IC80 | IC50 | IC80 |
| Our | A63-1.4 | Mono- | I | 1.380 | 7.280 | | 4.580 | 1.057 | 11.373 | 3.455 | 31.564 | 3.912 | 20.459 |
| bind- | | clonal | | | | | | | | | | | |
| ers | E12 | Mono- | I | >67 | >67 | 59.314 | >67 | >67 | >67 | >67 | >67 | >67 | >67 |
| | | clonal | | | | | | | | | | | |
| | E8 | Mono- | II | >67 | >67 | >67 | >67 | >67 | >67 | >67 | >67 | >67 | >67 |
| | | clonal | | | | | | | | | | | |
| | G1 | Mono- | III | >67 | >67 | >67 | >67 | >67 | >67 | >67 | >67 | >67 | >67 |
| | | clonal | | | | | | | | | | | |
| | A18-448 | Mono- | IV | | | | | | | | | | |
| | | clonal | | | | | | | | | | | |
| | B2 | Mono- | III | 1.517 | 5.755 | | 4.515 | | | | | | |
| | | clonal | | | | | | | | | | | |
| Com- | AZD31 | Mono- | I | >67 | >67 | >67 | >67 | >67 | >67 | >67 | >67 | >67 | >67 |
| peti- | 52 | clonal | | | | | | | | | | | |
| tion | ADG20 | Mono- | I/IV | >67 | >67 | >67 | >67 | >67 | >67 | >67 | >67 | >67 | >67 |
| | | clonal | | | | | | | | | | | |
| | BD55- | Mono- | I | | | | | | | | | | |
| | 1205 | clonal | | | | | | | | | | | |
| | VYD222 | Mono- | I/IV | 28.350 | 66.284 | 30.783 | >67 | 8.832 | 39.460 | 27.242 | >67 | >67 | >67 |
| | | clonal | | | | | | | | | | | |
| Our | MX2301 | Bi- | III + | | | | | | | | | | |
| mole- | | speci- | IV | | | | | | | | | | |
| cules | | fic | | | | | | | | | | | |
| | MX2148- | Tetra- | I + | 5.371 | 10.980 | 3.827 | 8.588 | 3.045 | 10.675 | 3.704 | 13.972 | 3.477 | 11.246 |
| | 006 | speci- | II + | | | | | | | | | | |
| | | fic | III + | | | | | | | | | | |
| | | | IV | | | | | | | | | | |
| | MX2673 | Tri- | I + | | | | | | | | | | |
| | | speci- | III + | | | | | | | | | | |
| | | fic | IV | | | | | | | | | | |
| | MX2674 | Bi- | I + | | | | | | | | | | |
| | | speci- | IV | | | | | | | | | | |
| | | fic | | | | | | | | | | | |

### Example 38

### Developability Assessment for MX2242, MX2265, and MX2301

The following biochemical and biophysical properties of the recited multispecific antibodies were assessed with the analytical assay and sample number or conditions indicated in parentheses:
overall purity (aSEC, DLS, DSC: initial samples without stress);
binding characterization (BLI (Octet) initial samples without stress);
LC MS and glycan profiling (LC-MS (intact and deglycosylated); unstressed samples);
surface hydrophobicity (aHIC (purity and hydrophobicity); initial samples without stress);
surface charge (pI profile) (iCIEF (charge heterogeneity and pI); initial samples without stress);
solubility at high concentration (target to approximately 100 mg/mL) (DLS, sSEC; high concentration samples);
thermal stability (Tₘ, Tₒₙₛₑₜ) (DSC: initial samples heated from 25° C., to 95° C.);
Thermal stress at 40° C. (aSEC, CD-SDS, binding potency; T0 time points (including T0 as control+T1 and T2 weeks));
Freeze thaw 5× (DLS, aSEC, CE-SDS; freeze and thaw 5 cycles);
pH 3.5 stress (RT for 4 hours) (aSEC, CE-SDS, binding potency; T0 as control plus 4 hours stress);
pH 9.0 stress (RT for 24 hours) (aSEC, CE-SDS, binding potency; T0 as control plus 24 hours stress;
serum/PBS 37° C., stability (aSEC and relative potency analysis (Octeta); T0, d1, d2, d5 and d7;
polyreactivity (ELISA based polyreactivity, compared to benchmark mAbs); and
Self association (AC-SINS to evaluate Ab self-interaction: characterizing antibody self-association).

The data generated for any particular multispecific was utilized to assess developability for clinical development purposes.

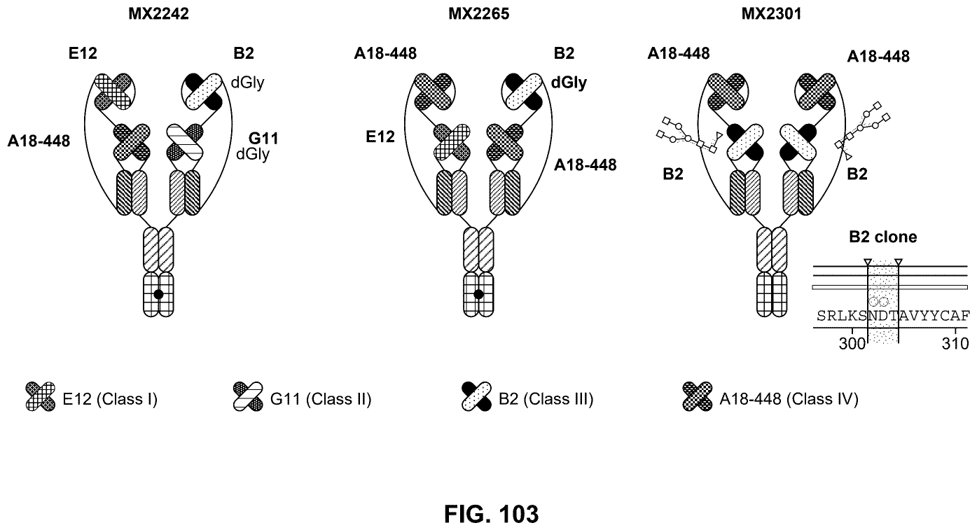

Buffer Exchange Recovery: MX2242, MX2265, and MX2301 (FIG. 103) were formulated in formulation buffer, 20 mM Histidine (pH 6.0), 8% Sucrose, MX2242, MX2265, and MX2301 were measured pre- and post-dialysis. Recovery and aSEC purity were assessed and are shown in Table E54. White precipitate was observed pre- and post-dialysis for MX2242.

TABLE E54
Buffer Exchange Recovery of MX2242, MX2265, and MX2301.

| | (mg) pre- | (mg) post- | % | aSEC |
|---|---|---|---|---|
| Molecule | dialysis | dialysis | Recovery | purity % |
| MX2242* | 12.35 | 9 | 72.8 | 99.1 |
| MX2265 | 11.8 | 11.6 | 98.5 | 92.8 |
| MX2301 | 14.5 | 12.6 | 86.9 | 98 |

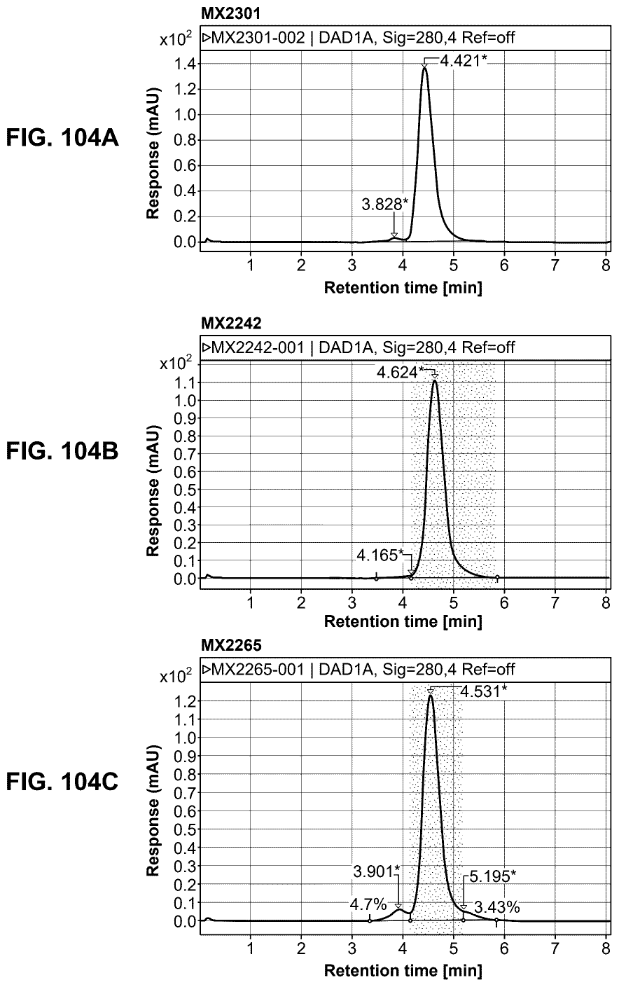

Purity Assessment by HPLC aSEC: All MX2242, MX2265, and MX2301 samples were formulated at 1 mg/ml in 20 mM Histidine pH 6.0, 8% sucrose, HPLC aSEC profiles are shown in FIGS. 104A-104C. Purity % is shown in Table E55. All tested molecules showed good aSEC purity, MX2265 showed a lower aSEC purity (about 93%).

TABLE E55
Purity Assessment by HPLC aSEC
of MX2242, MX2265, and MX2301.

| ID | Purity % |
|---|---|
| MX2242 | 99.2% |
| MX2265 | 92.9% |
| MX2301 | 98% |

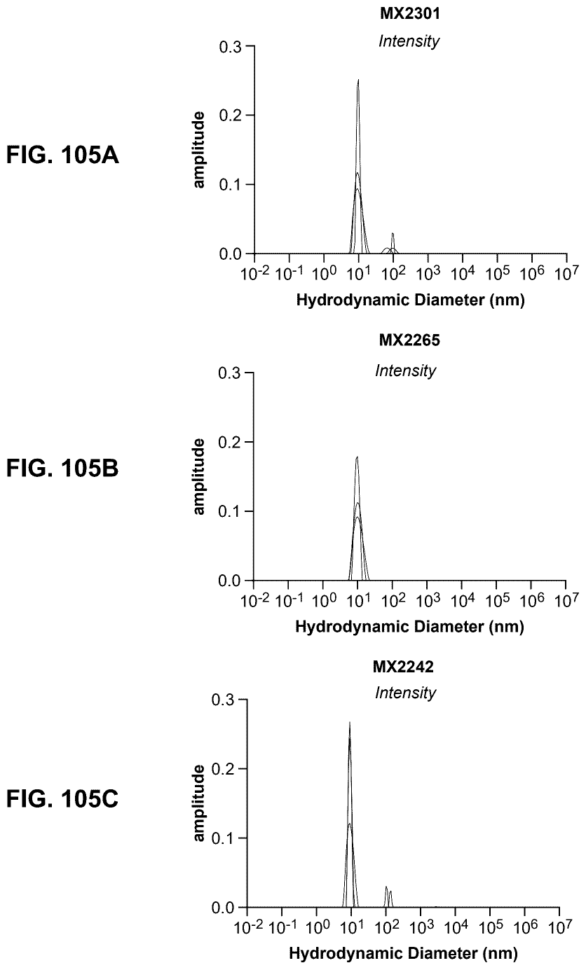

Purity Assessment by DLS: Sizing and polydispersity of MX2242, MX2265, and MX2301 were analyzed by DLS (UNCLE, Acute BioScience). Each molecule was tested in 3 experimental replicates (all shown in FIGS. 105A-105C). Sizing was adjusted by buffer viscosity. All molecules showed the expected hydrodynamic diameter, PDI values were relatively high (expected≤0.10), indicating the presence of sample heterogeneity (Table E56).

TABLE E56
Purity Assessment by DLS of MX2242, MX2265, and MX2301.

| | Z-Ave. | | Pk 1 Peak | Pk 1 Mass |
|---|---|---|---|---|
| Sample | Dia. (nm) | PDI | Dia. (nm) | (%) |
| 1 mg/ml ref MX2242 | 8.28 ± 0.14 | 0.28 ± 0.00 | 9.54 ± 0.00 | 99.99 ± 0.01 |
| 1 mg/ml ref MX2265 | 8.70 ± 0.65 | 0.29 ± 0.01 | 10.34 ± 0.00 | 100.00 ± 0.00 |
| 1 mg/ml ref MX2301 | 8.96 ± 0.17 | 0.29 ± 0.00 | 9.81 ± 0.38 | 99.97 ± 0.01 |

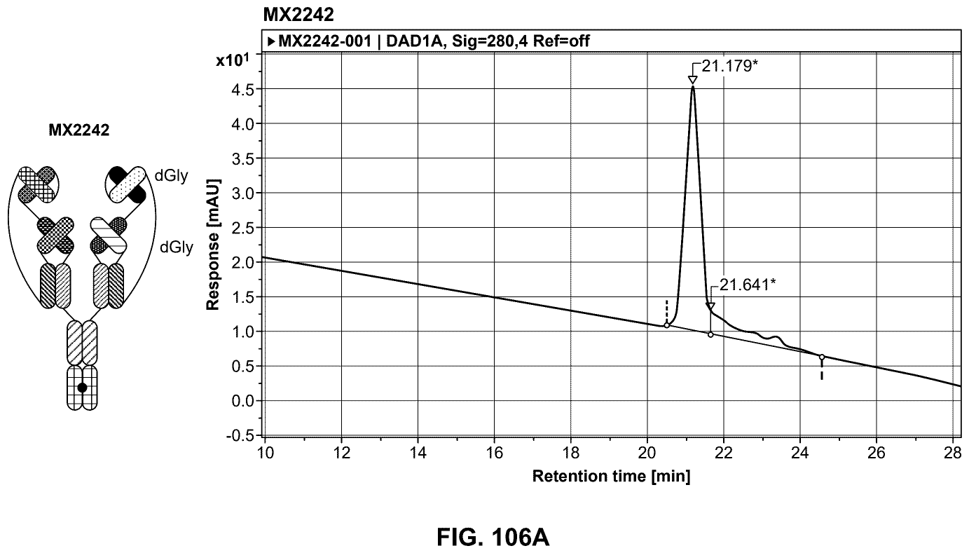

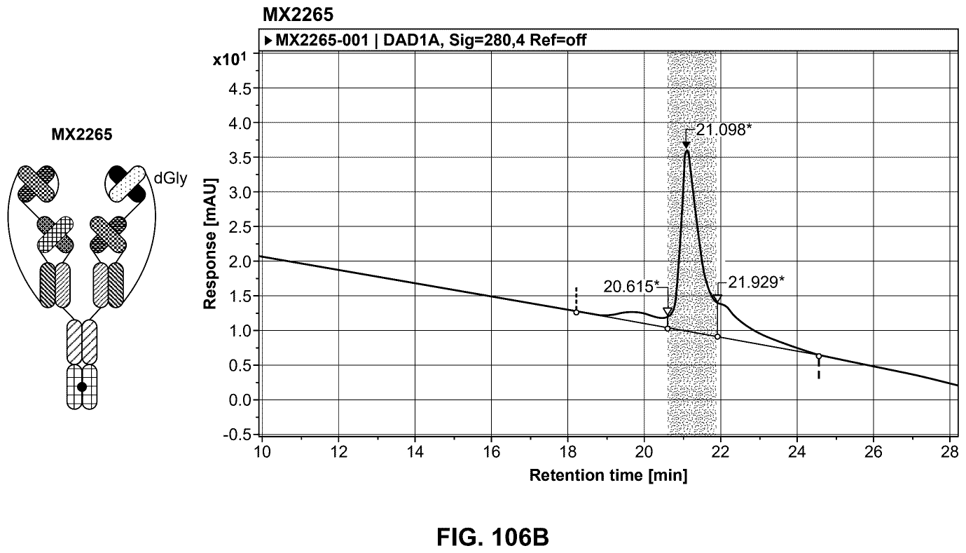

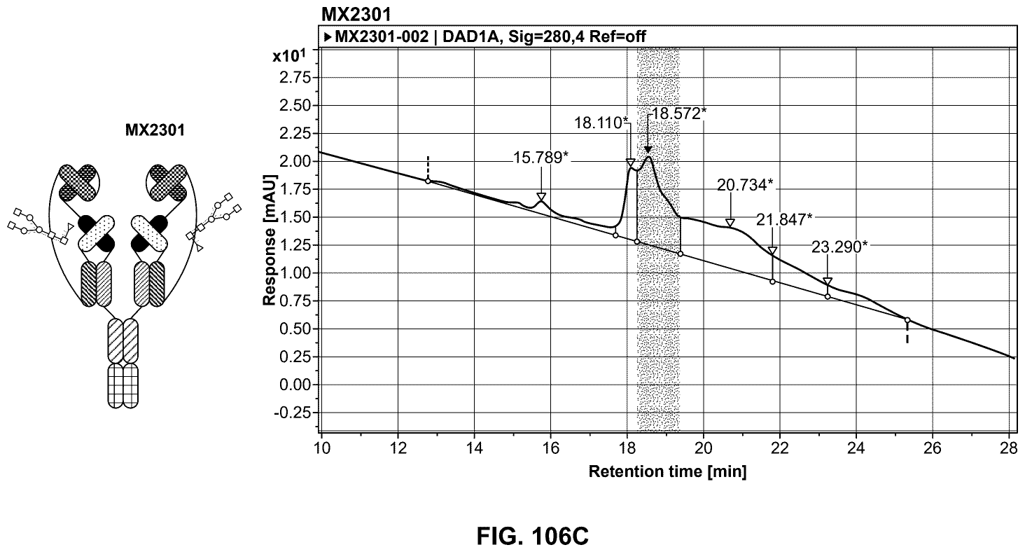

Surface Hydrophobicity Analysis by aHIC: Surface Hydrophobicity of MX2242, MX2265, and MX2301 was analyzed by HPLC aHIC, HPLC aHIC analysis of MX2242 (tetra-specific) and MX2265 (tri-specific) showed a major peak (71-81%) that eluted at about 21 minutes towards the end of salt gradient, indicating relatively high surface hydrophobicity (FIGS. 106A-106B and Table E57), HPLC aHIC analysis of MX2301 (bi-specific) showed a polydispersed aHIC profile, suggesting the presence of surface hydrophobicity heterogeneity (FIG. 106C and Table E58).

TABLE E57
Surface Hydrophobicity Analysis
by aHIC of MX2242 and MX2265.

HPLC_HIC

| ID | Retention time | Purity (major peak) |
|---|---|---|
| MX2242 | 21.18 min | 81.2% |
| MX2265 | 21.09 min | 71.2% |

TABLE E58
Surface Hydrophobicity Analysis by aHIC of MX2301.

| RT (min) | Area % |
|---|---|
| 15.789 | 6.852 |
| 18.110 | 11.022 |
| 18.572 | 29.242 |
| 20.734 | 36.251 |
| 21.847 | 10.720 |
| 23.290 | 5.912 |

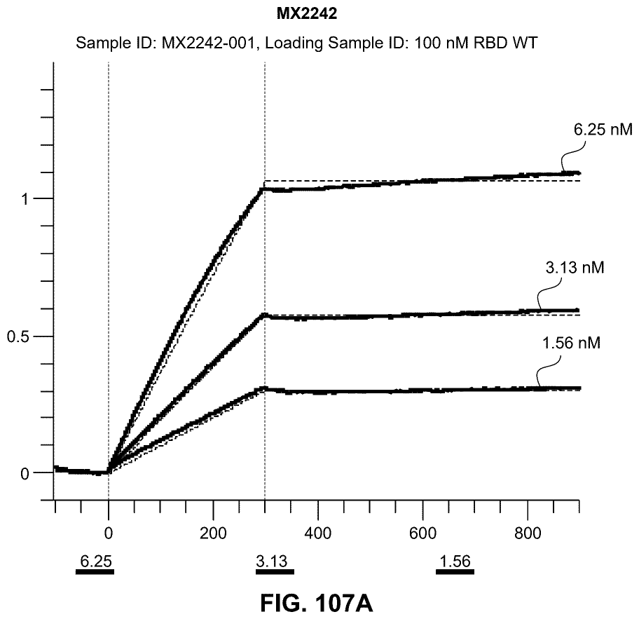

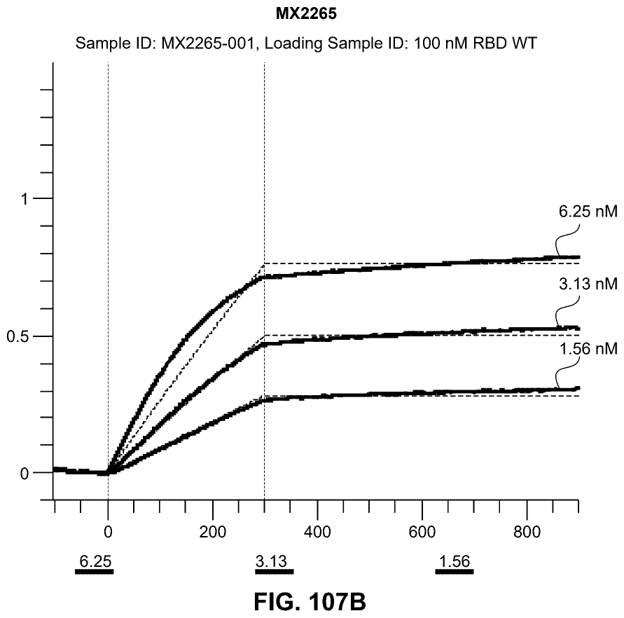

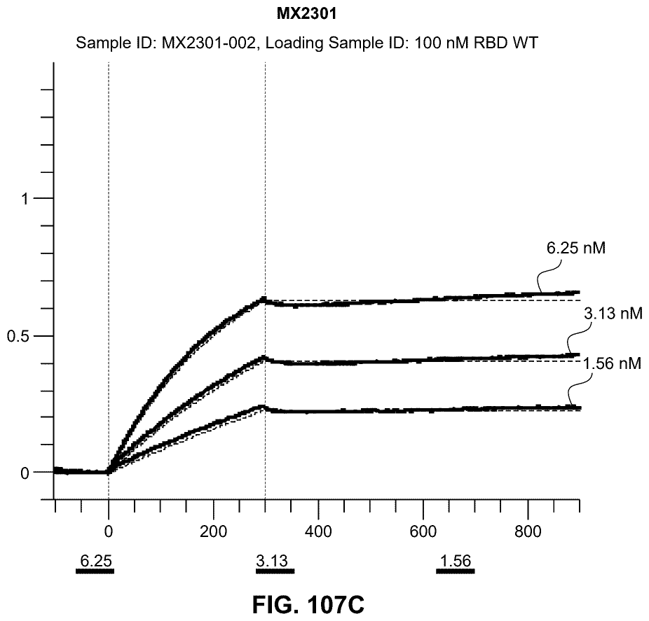

Binding Characterization by BLI: Binding properties of MX2242, MX2265, and MX2301 were analyzed by BLI (Octet R8, Sartorius). Specifically, binding to COVID wild-type RBD was analyzed. The apparent binding affinity was in the sub-nanomolar range (FIGS. 107A-107C, and Table E59).

TABLE E59
Binding Characterization by BLI
of MX2242, MX2265, and MX2301.

| Solution Analyte | Loaded Ligand | KD (nM) |
|---|---|---|
| MX2242 | COVID RBD WT | ≤0.01 |
| MX2265 | COVID RBD WT | ≤0.01 |
| MX2301 | COVID RBD WT | ≤0.01 |

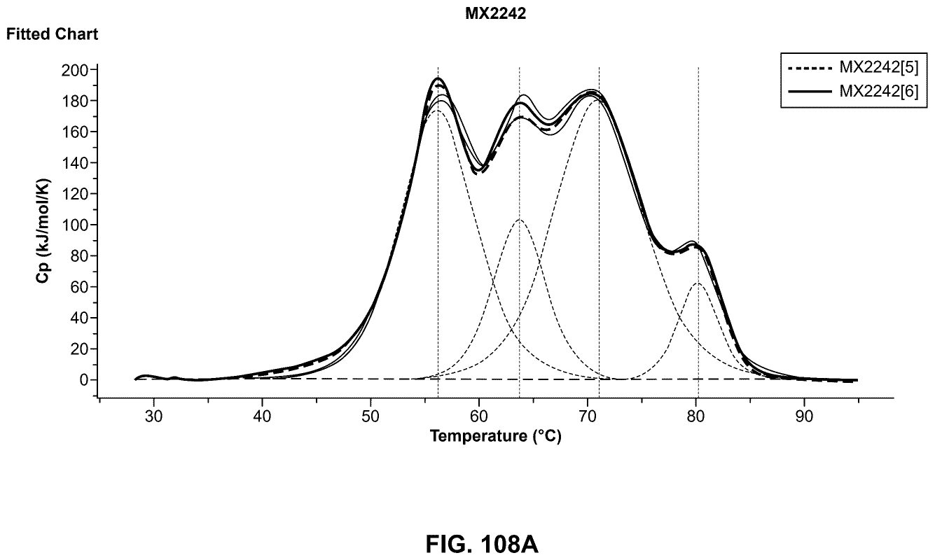

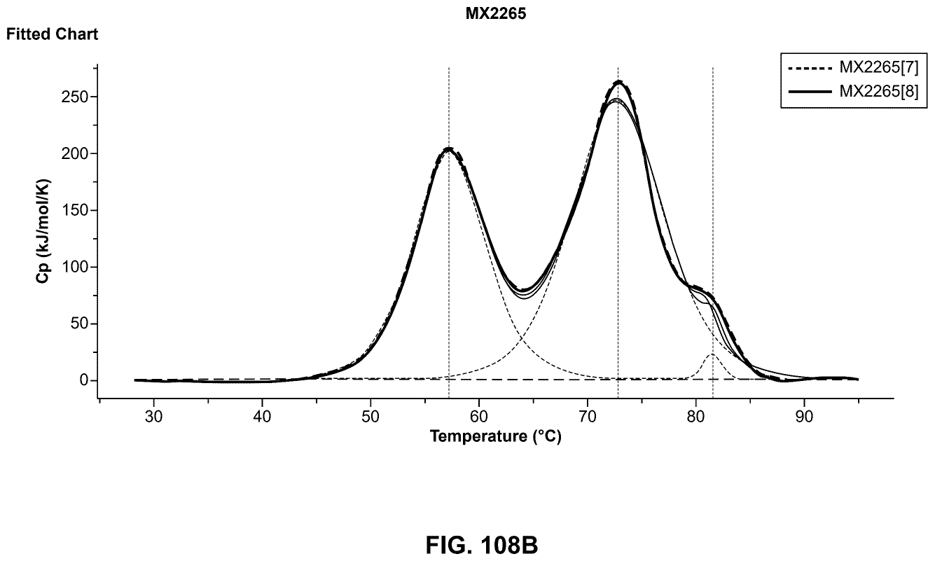

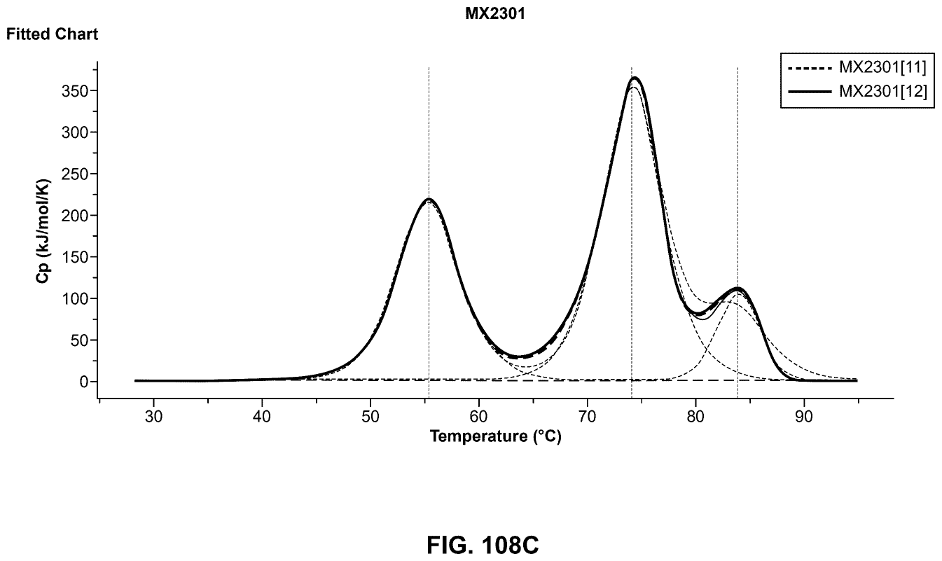

Thermostability Analysis by DSC: Thermostability of MX2242, MX2265, and MX2301 was analyzed by DSC, MX2242, MX2265, and MX2301 were prepared at 0.5 mg/ml in 20 mM Histidine pH 6.0, 8% sucrose. Results indicated a non-two-state unfolding process. Each molecule was analyzed in two experimental replicates (both shown in FIGS. 108A-108C). Thermostability is shown in Table E60. The tested molecules showed lower thermal stability.

TABLE E60
Thermostability Analysis by DSC of MX2242, MX2265, and MX2301.

| | T_{Onset} (° C.) | Tₘ_{¹} (° C.) | Tₘ_{²} (° C.) | Tₘ_{³} (° C.) | Tₘ_{⁴} (° C.) |
|---|---|---|---|---|---|
| MX2242 | 39.79 ± 0.65 | 56.28 ± 0.10 | 63.72 ± 0.04 | 70.62 ± 0.40 | 80.22 ± 0.1 |
| MX2265 | 46.23 ± 0.54 | 57.14 ± 0.01 | 72.74 ± 0.00 | 81.20 ± 0.28 | |
| MX2301 | 44.78 ± 0.10 | 55.31 ± 0.02 | 74.13 ± 0.05 | 83.76 ± 0.09 | |

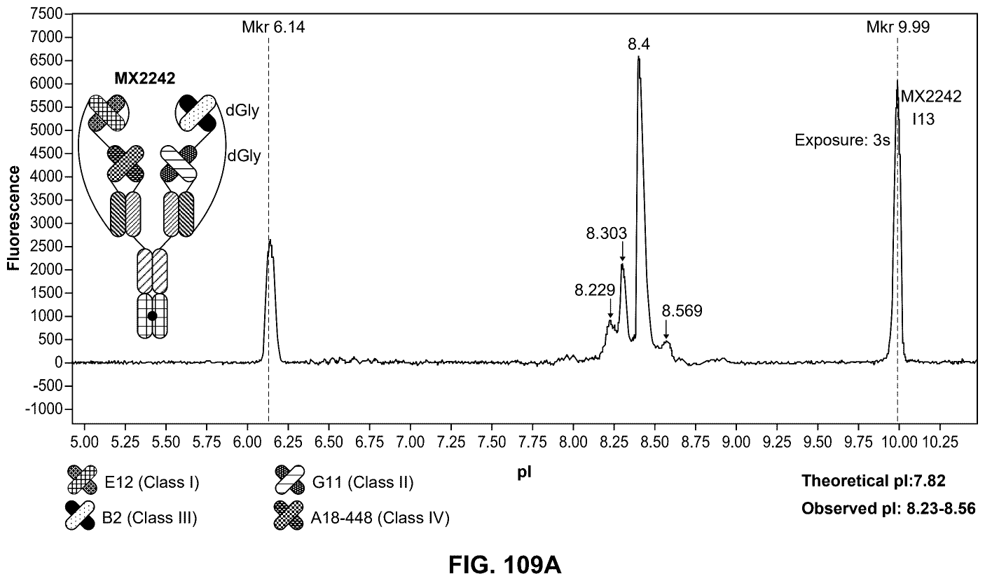

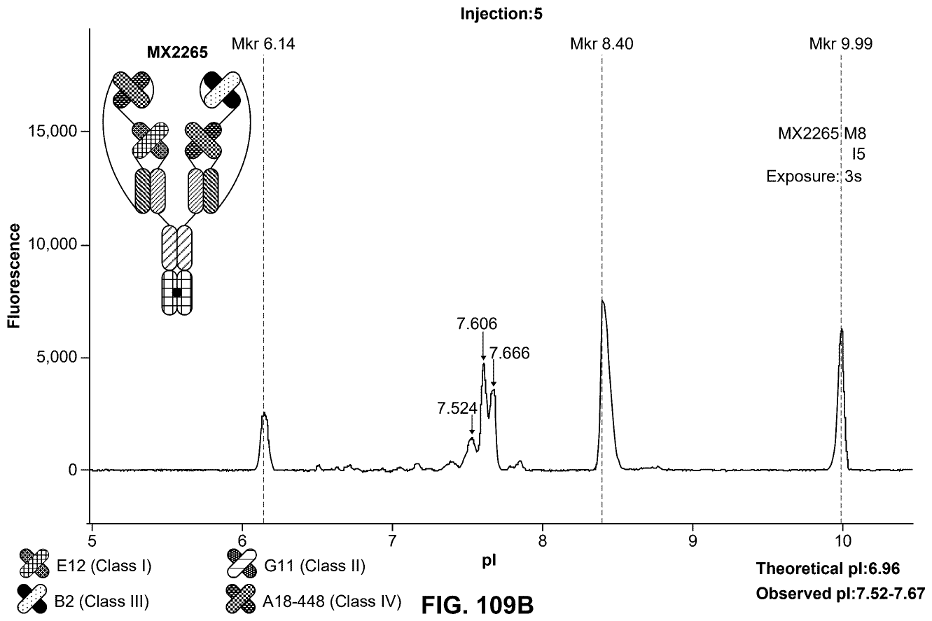

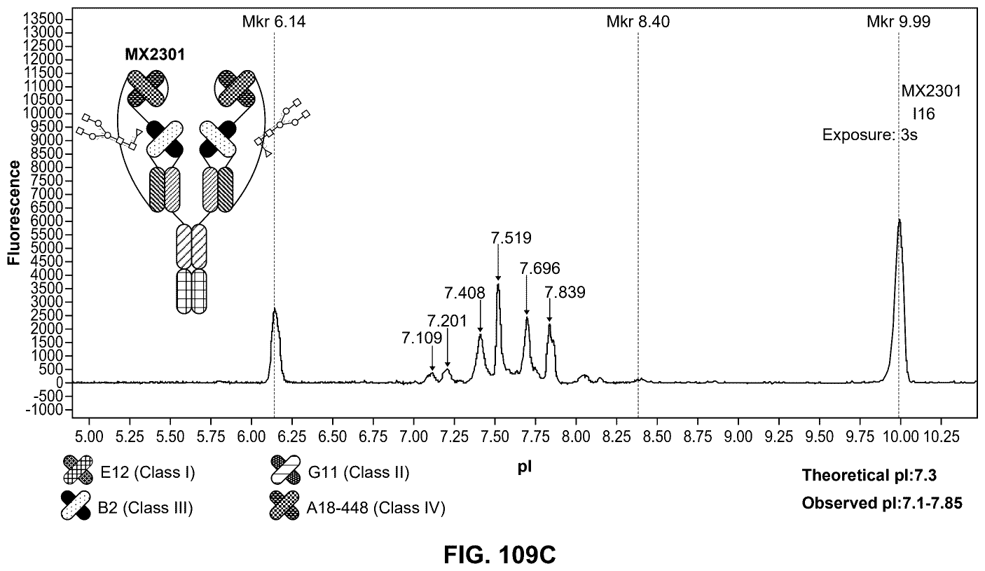

Surface Charge (pI profile) cIEF Analysis: The isoelectric point (pI) of MX2242, MX2265, and MX2301 was measured by cIEF (FIGS. 109A-109C). The measured pI of MX2242 was is in the desired range of 8.23-8.56 (Table E61), and MX2242 exhibited an appreciable amount of surface charge heterogeneity. The measured pI of MX2265 was in a range of from 7.52 to 7.67 (Table E62), and showed surface charge heterogeneity. The measured pI of MX2301 was in a range of from 7.1 to 7.85 (Table E63), and showed the surface charge heterogeneity.

TABLE E61
cIEF Analysis (pI) of MX2242.

| Peak # | Calculated pI | Abundance (%) |
|---|---|---|
| 1 | 8.229 | 14.53 |
| 2 | 8.303 | 31.16 |
| 3 | 8.400 | 49.17 |
| 4 | 8.559 | 5.13 |

TABLE E62
cIEF Analysis (pI) of MX2265.

| Peak # | Calculated pI | Abundance (%) |
|---|---|---|
| 1 | 7.524 | 18.27 |
| 2 | 7.606 | 47.33 |
| 3 | 7.666 | 34.40 |

TABLE E63
cIEF Analysis (pI) of MX2301.

| Peak # | Calculated pI | Abundance (%) |
|---|---|---|
| 1 | 7.109 | 3.06 |
| 2 | 7.201 | 5.04 |
| 3 | 7.408 | 19.88 |
| 4 | 7.519 | 27.23 |
| 5 | 7.696 | 23.91 |
| 6 | 7.839 | 20.89 |

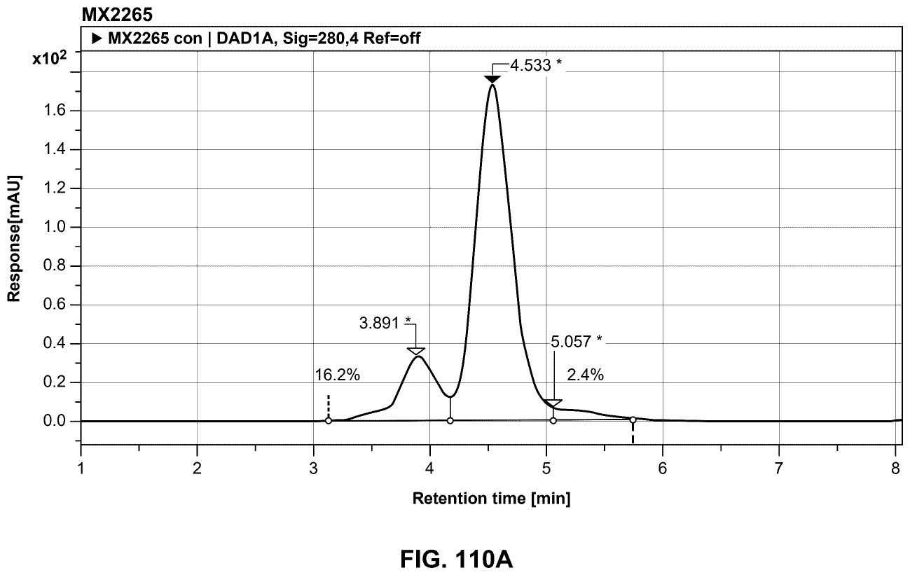

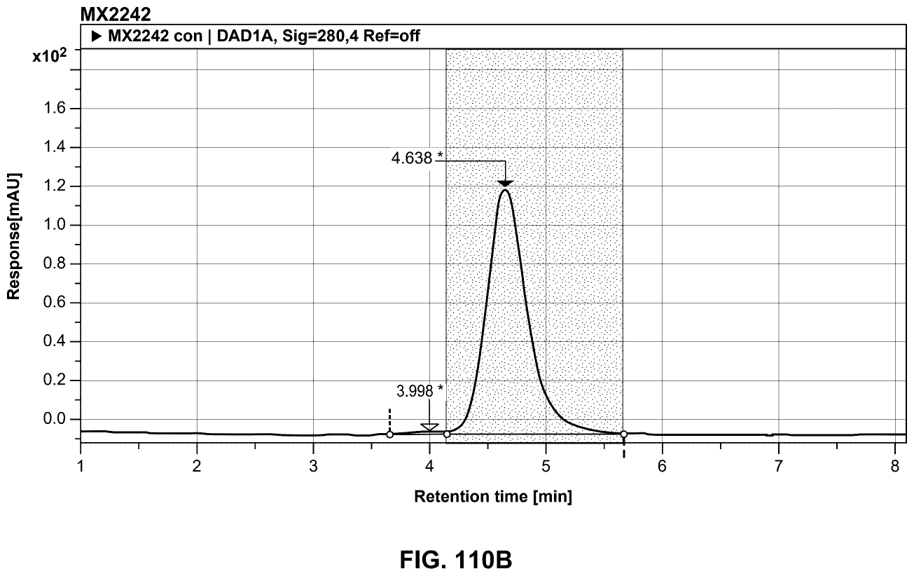

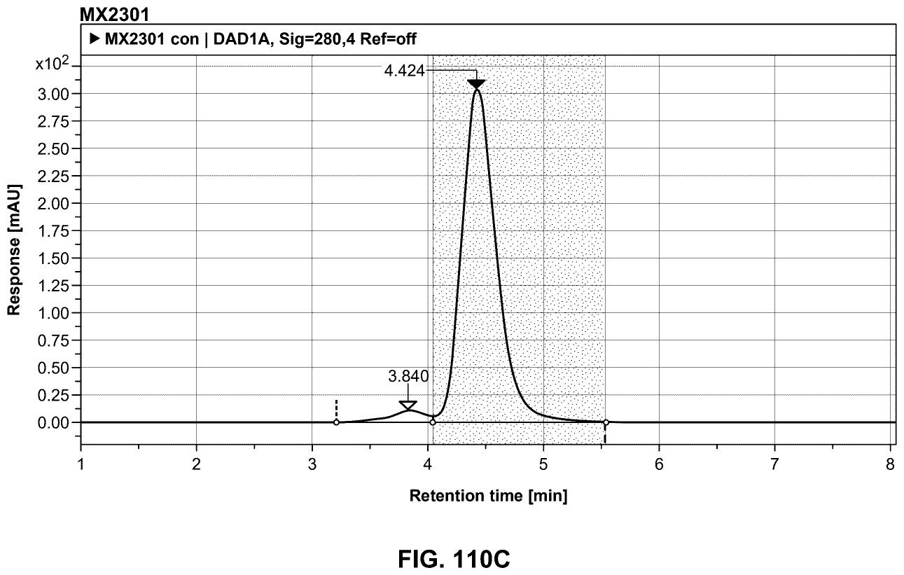

Solubility at High Concentration (aSEC): Solubility at high concentration of MX2242, MX2265, and MX2301 was measured by aSEC. For all molecules tested, good recovery was obtained during the process of concentration adjustment, MX2242 and MX2301 showed good overall aSEC purity, MX2265 increased high molecular weight species (HMW) by 11.5% after concentration (FIGS. 110A-110C and Table E64).

TABLE E64
Solubility at high concentration (aSEC)
of MX2242, MX2265, and MX2301.

| ID | Concentration mg/ml | Recovery % | Purity % |
|---|---|---|---|
| MX2242 | 74.6 | 86% | 99.2% |
| MX2265 | 119 | 91% | 81.2% |
| MX2301 | 99 | 96% | 96.2% |

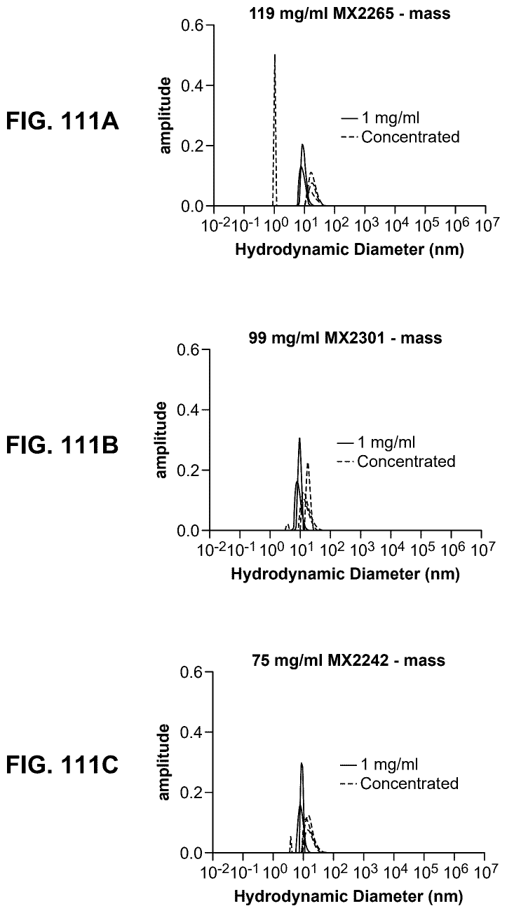

Solubility at High Concentration (DLS): Solubility at high concentration of MX2242, MX2265, and MX2301 was also measured by DLS. All samples tested showed significantly increased hydrodynamic diameter at high concentrations (FIGS. 111A-111C and Table E65), MX2301 demonstrated a slightly better DLS profile at high concentration than MX2242 and MX2265.

TABLE E65
Solubility at high concentration (DLS) of MX2242, MX2265, and MX2301.

| | Z-Ave. | | Pk 1 Peak | Pk 1 Mass |
|---|---|---|---|---|
| Sample | Dia. (nm) | PDI | Dia. (nm) | (%) |
| 1 mg/ml ref MX2242 | 8.28 ± 0.14 | 0.28 ± 0.00 | 9.54 ± 0.00 | 99.99 ± 0.01 |
| 75 mg/ml MX2242 | 67.70 ± 0.70 | 0.36 ± 0.00 | 21.71 ± 2.34 | 99.47 ± 0.16 |
| 1 mg/ml ref MX2265 | 8.70 ± 0.65 | 0.29 ± 0.01 | 10.34 ± 0.00 | 100.00 ± 0.00 |
| 119 mg/ml MX2265 | 75.34 ± 6.23 | 0.28 ± 0.02 | 24.40 ± 1.89 | 99.96 ± 0.25 |
| 1 mg/ml ref MX2301 | 8.96 ± 0.17 | 0.29 ± 0.00 | 9.81 ± 0.38 | 99.97 ± 0.01 |
| 99 mg/ml MX2301 | 41.71 ± 5.16 | 0.26 ± 0.03 | 20.84 ± 2.75 | 99.55 ± 0.37 |

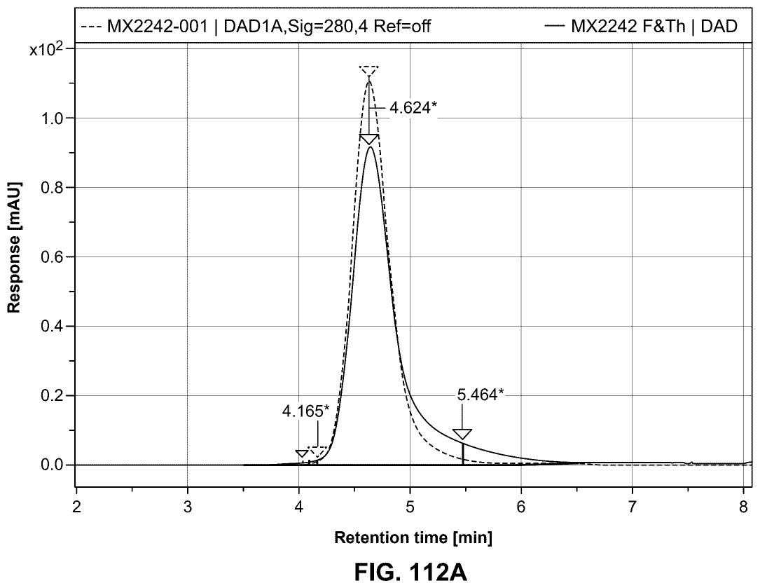

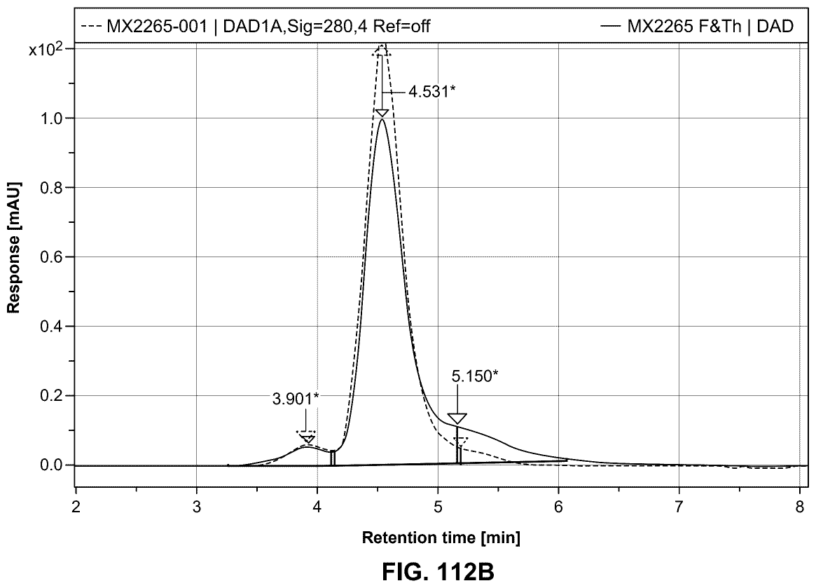

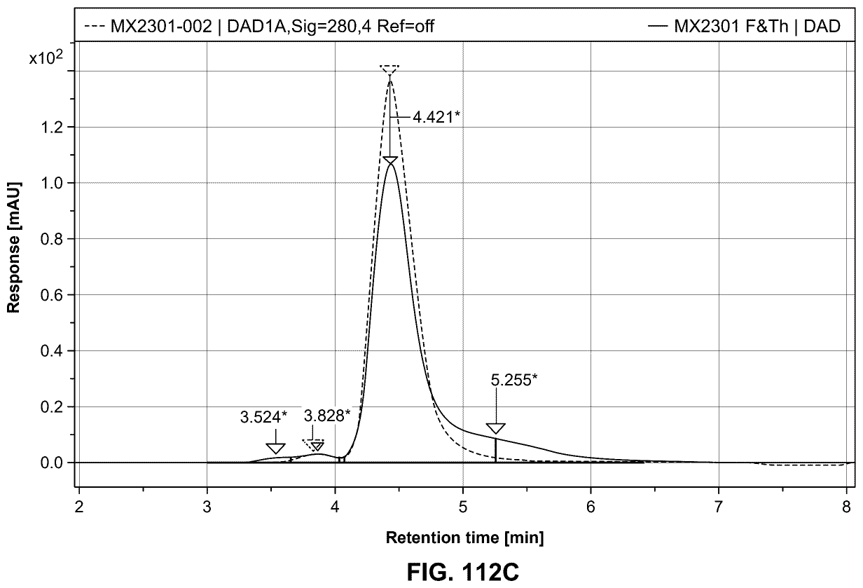

Analysis under Freeze and Thaw (HPLC-aSEC): HPLC-aSEC analysis under 5 freeze and thaw cycles was performed for MX2242, MX2265, and MX2301. The tested samples showed an increase in the LMW (low molecular weight) species by 4-8% (FIGS. 112A-112C, Table E66). Similar aSEC profiles were observed with additional 0.02% PS80.

TABLE E66
HPLC-aSEC analysis under 5 freeze and thaw
cycles of MX2242, MX2265, and MX2301.

| ID | LMW % | Purity % |
|---|---|---|
| MX2242 | 4.1% | 95.4% |
| MX2265 | 9.4% | 86.3% |
| MX2301 | 6.9% | 90% |

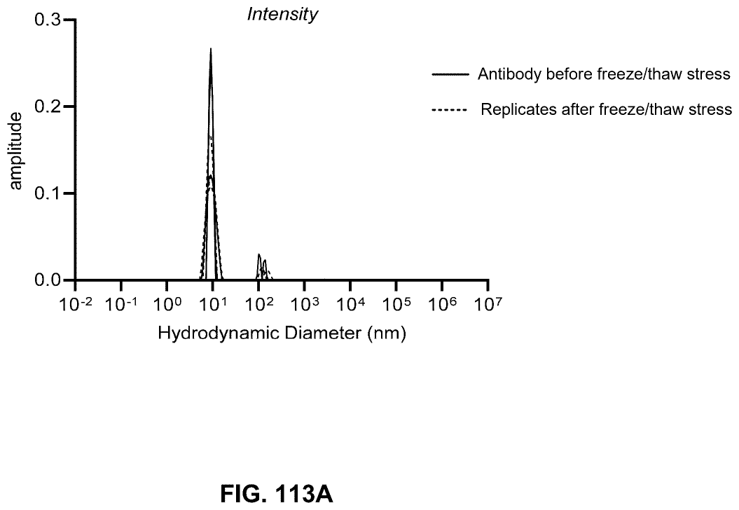

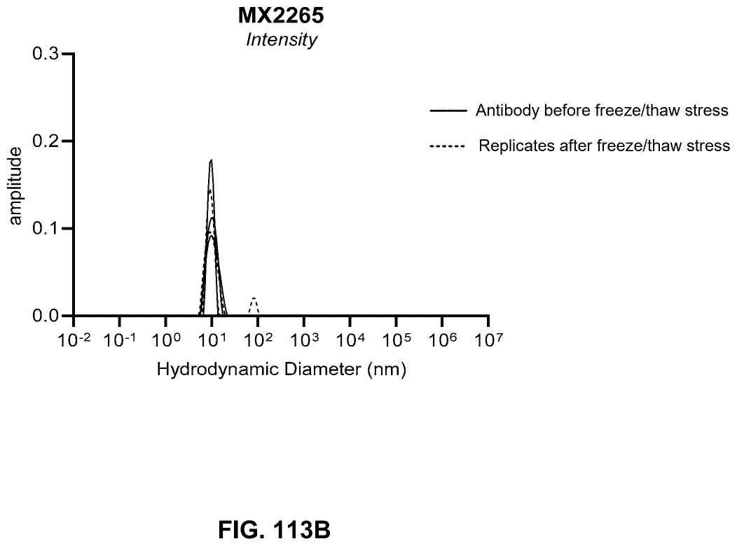

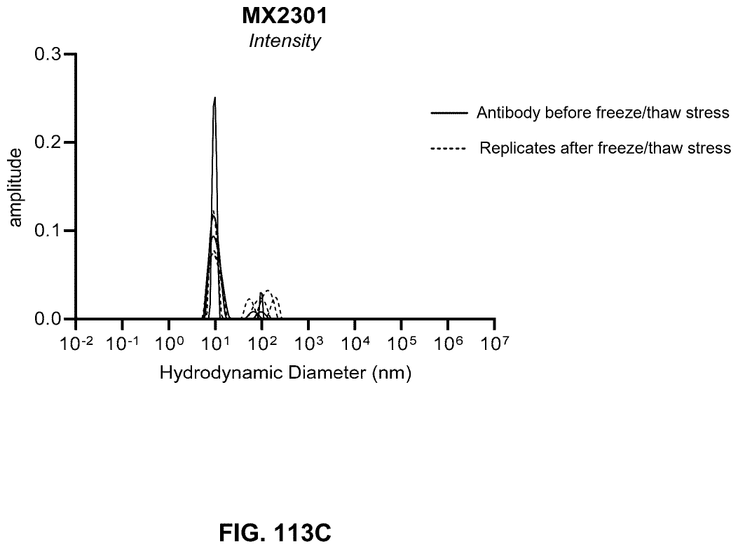

Analysis under Freeze and Thaw (DLS): Sizing and polydispersity under freeze and thaw cycles were analyzed for MX2242, MX2265, and MX2301 by DLS (UNCLE, Acute BioScience) (FIGS. 113A-113C). Five freeze/thaw cycles were performed with addition of 0.02% PS80, MX2301 showed a slight increase in hydrodynamic diameter after the freeze/thaw stress, while MX2242 and MX2265 showed essentially no change (FIGS. 113A-113C, Table E67).

TABLE E67
DLS analysis under 5 freeze and thaw
cycles of MX2242, MX2265, and MX2301.

| | Z-Ave. | | Pk 1 Peak | Pk 1 Mass |
|---|---|---|---|---|
| Sample | Dia. (nm) | PDI | Dia. (nm) | (%) |
| 1 mg/ml ref MX2242 | 8.28 ± 0.14 | 0.28 ± 0.00 | 9.54 ± 0.00 | 99.99 ± 0.01 |
| Freeze/thaw MX2242 | 8.45 ± 0.24 | 0.28 ± 0.00 | 9.29 ± 0.35 | 100.00 ± 0.00 |
| 1 mg/ml ref MX2265 | 8.70 ± 0.65 | 0.29 ± 0.01 | 10.34 ± 0.00 | 100.00 ± 0.00 |
| Freeze/thaw MX2265 | 8.96 ± 0.23 | 0.28 ± 0.00 | 9.29 ± 0.35 | 99.96 ± 0.03 |
| 1 mg/ml ref MX2301 | 8.96 ± 0.17 | 0.29 ± 0.00 | 9.81 ± 0.38 | 99.97 ± 0.01 |
| Freeze/thaw MX2301 | 12.31 ± 0.86 | 0.25 ± 0.01 | 9.66 ± 0.17 | 99.86 ± 0.09 |

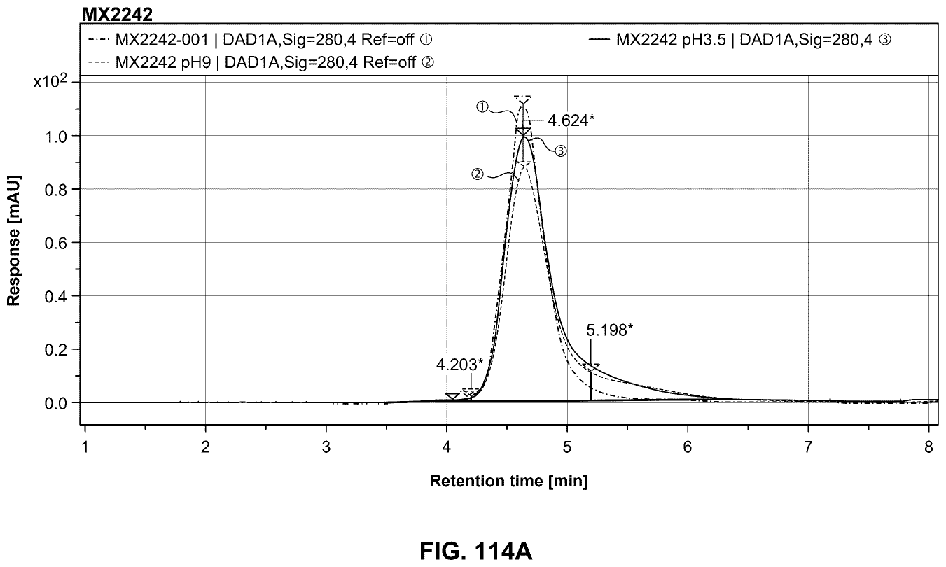

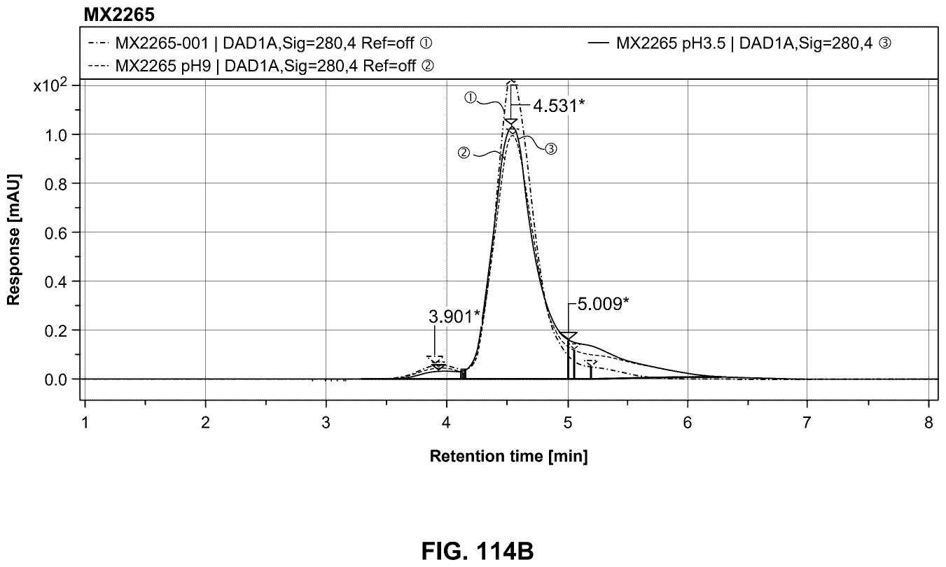

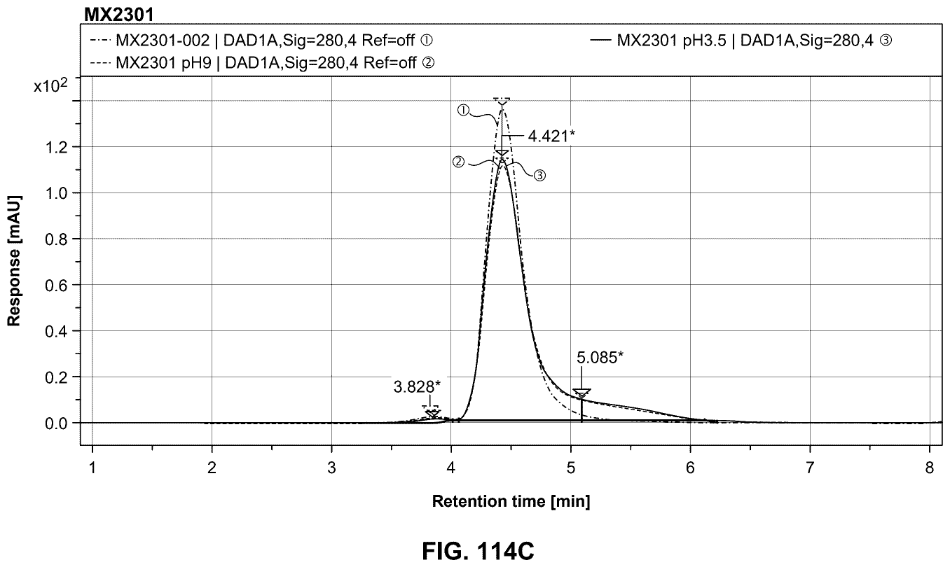

Analysis under pH stress (aSEC): MX2242, MX2265, and MX2301 were analyzed under pH stress by aSEC at pH 3.5 and pH 9. All tested molecules showed increased peak tailing under pH stress by aSEC (FIGS. 114A-114C, Tables E68-E70).

TABLE E68
aSEC analysis under pH stress of MX2242.

| pH stress | Increased LMW % |
|---|---|
| pH 3.5 | 14% |
| pH 9 | 7.1% |

TABLE E69
aSEC analysis under pH stress of MX2301.

| pH stress | Increased LMW % |
|---|---|
| pH3.5 | 9.7% |
| pH9 | 9.3% |

TABLE E70
aSEC analysis under pH stress of MX2265.

| pH stress | Increased LMW % |
|---|---|
| pH3.5 | 15% |
| pH9 | 12.4% |

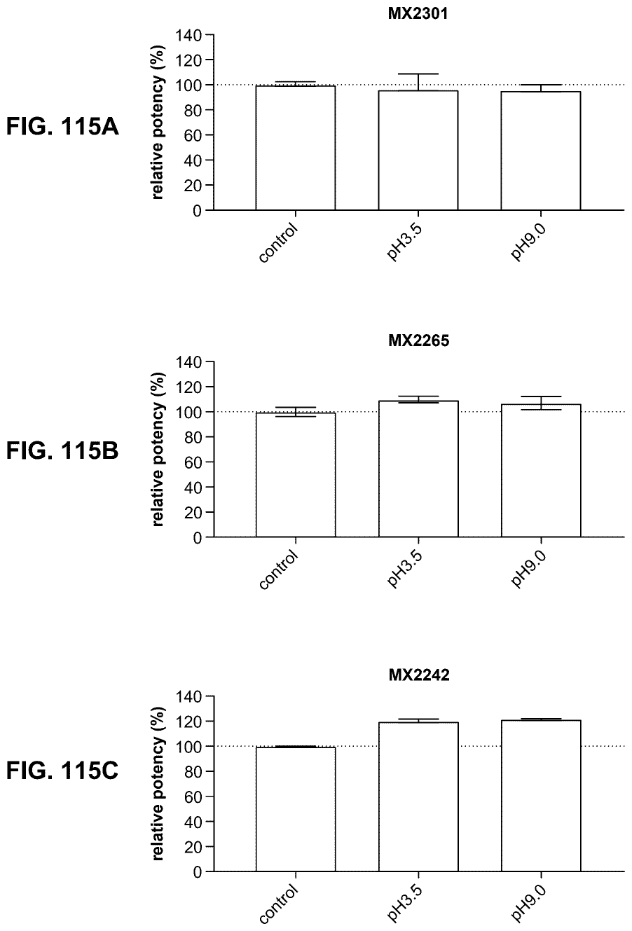

Potency Analysis after pH Stress: Potency after pH stress at pH 3.5 and pH 9.0 was analyzed for MX2242, MX2265, and MX2301 by BLI, pH 9.0 stress was for 24 hours and pH 3.5 stress was for 4 hours. Relative potency was determined by binding response by BLI to COVID WT (wild type) RBD. No changes in kinetics were observed. All molecules preserve their activities after pH 3.5 or pH 9.0 stress (FIGS. 115A-115C).

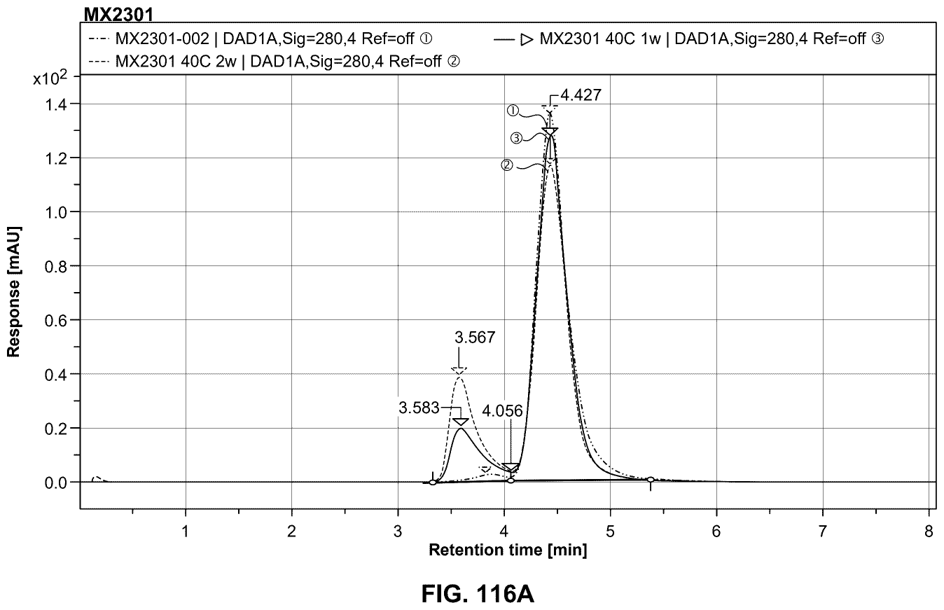

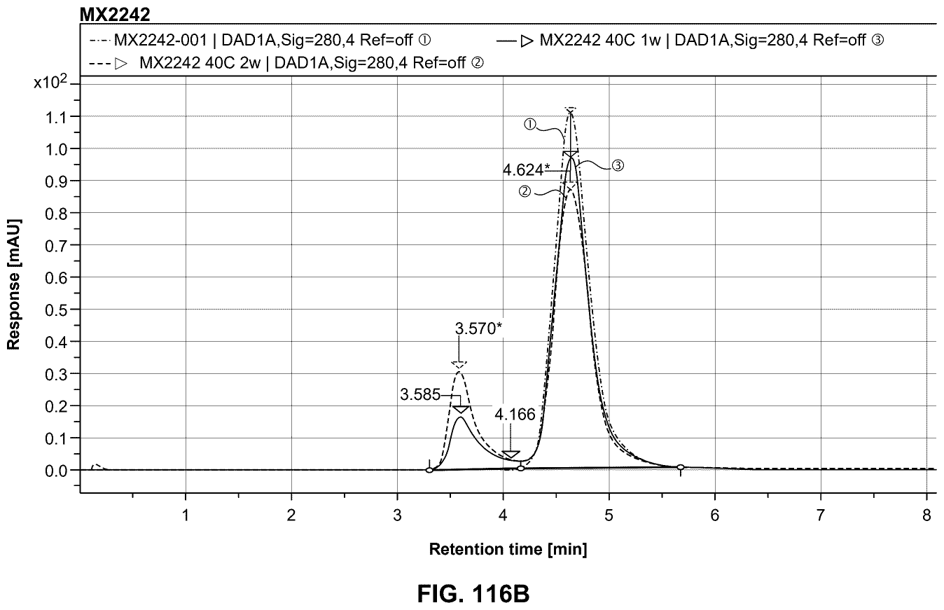

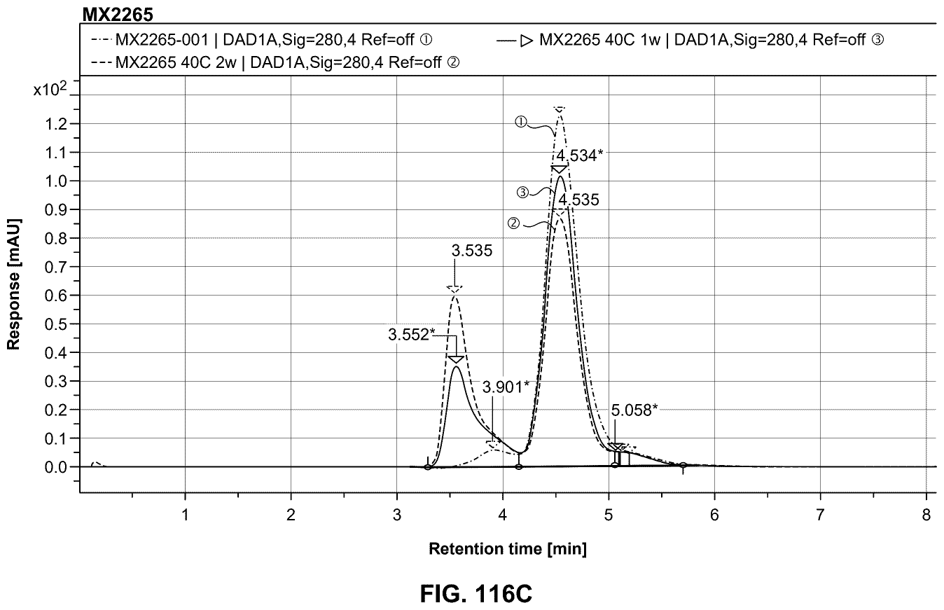

Thermal Stress Analysis (40° C.): Thermostability of MX2242, MX2265, and MX2301 was tested by aSEC after incubation at 40° C., for up to 2 weeks. Incubation at 40° C., for up to 2 weeks resulted in significantly increased HMW % (high molecular weight %) in all 3 tested molecules (FIGS. 116A-116C, Tables E71-E73).

TABLE E71
aSEC analysis under thermal stress of MX2242.

| 40° C. stress | Increased HMW % |
|---|---|
| 1 week | 12.8% |
| 2 weeks | 21.5% |

TABLE E72
aSEC analysis under thermal stress of MX2301.

| 40° C. stress | Increased HMW % |
|---|---|
| 1 week | 13.3% |
| 2 weeks | 22.7% |

TABLE E73
aSEC analysis under thermal stress of MX2265.

| 40° C. stress | Increased HMW % |
|---|---|
| 1 week | 25.2% |
| 2 weeks | 36.6% |

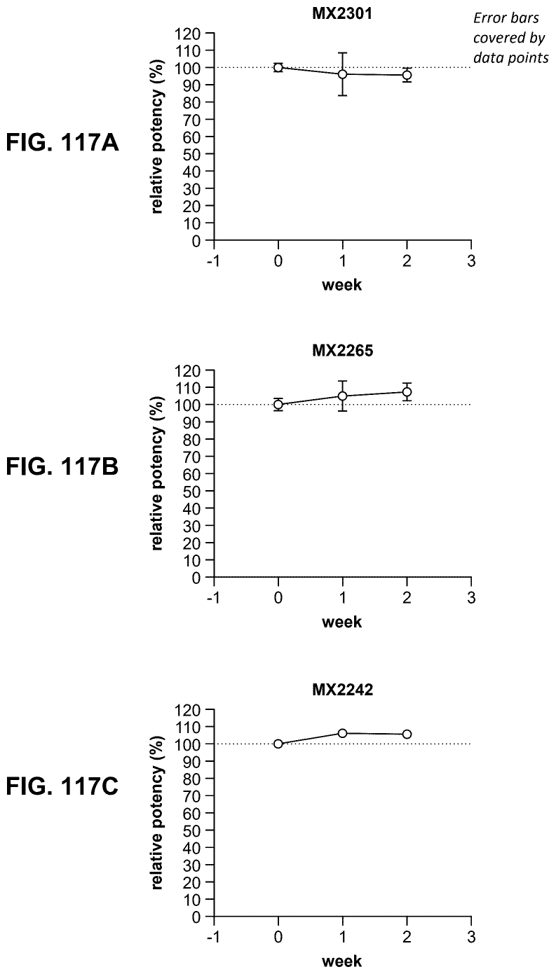

Analysis of Potency after Thermal Stress: Potency of MX2242, MX2265, and MX2301 after thermal stress was analyzed after incubation at 40° C., in formulation buffer for 2 weeks. Relative potency was determined from binding response by BLI to COVID WT (wild type) RBD. No changes in kinetics were observed. All tested molecules preserve their binding activities throughout the 2 weeks at 40° C., stress (FIGS. 117A-117C).

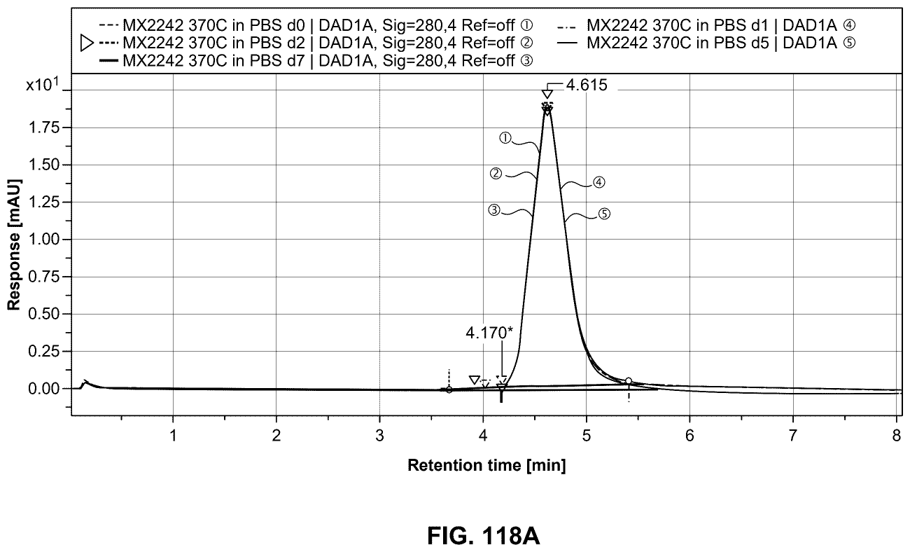

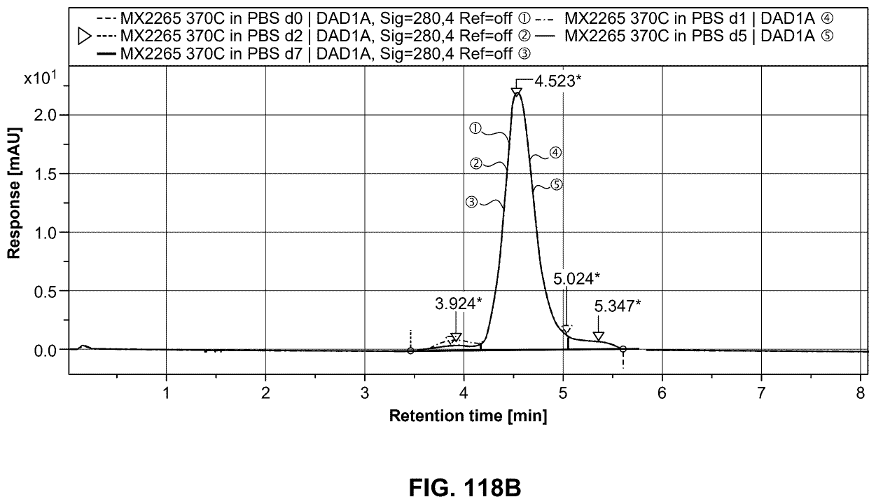

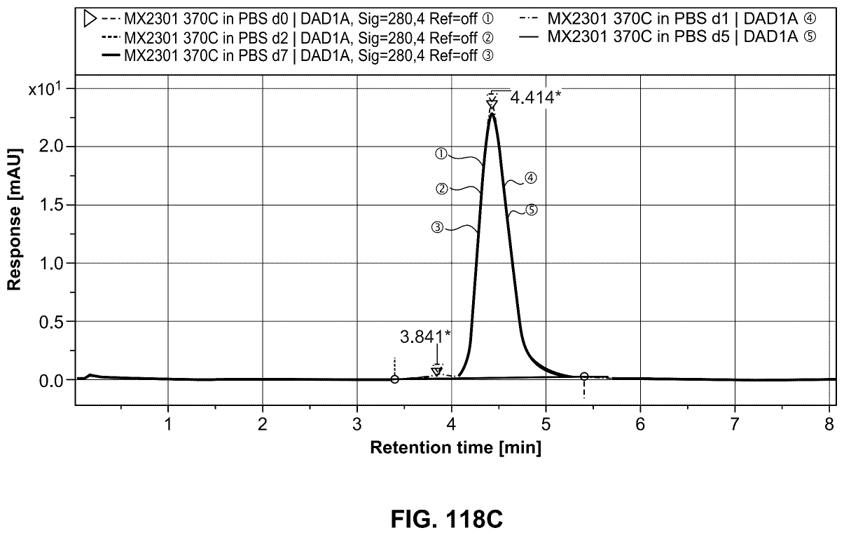

Thermal Stress Analysis (37° C.): Thermostability of MX2242, MX2265, and MX2301 was tested by aSEC after incubation at 37° C. All samples were diluted in PBS to 0.2 mg/ml and incubated at 37° C., for up to 1 week. All tested samples showed stable aSEC profiles under PBS incubation at 37° C., for up to 7 days (FIGS. 118A-118C).

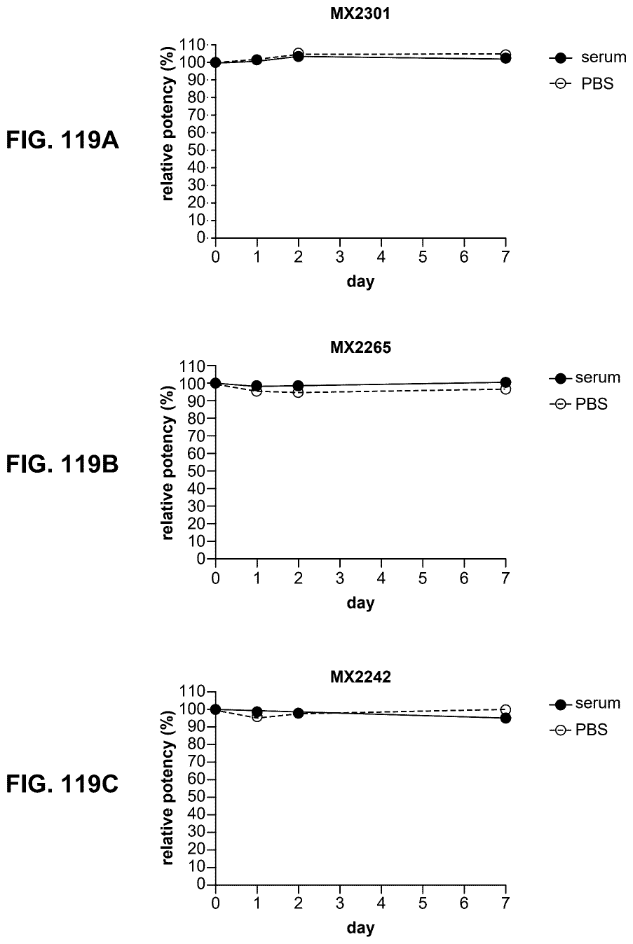

Potency Analysis after Serum Incubation: Potency after serum incubation was analyzed for MX2242, MX2265, and MX2301. All samples were incubated in PBS or final human serum 0.8× at 37° C., for 0-7 days. Relative potency was determined by BLI from binding to COVID RBD WT. All molecules maintained anti-RBD WT binding activity throughout the 7-day serum or PBS incubation (FIGS. 119A-119C).

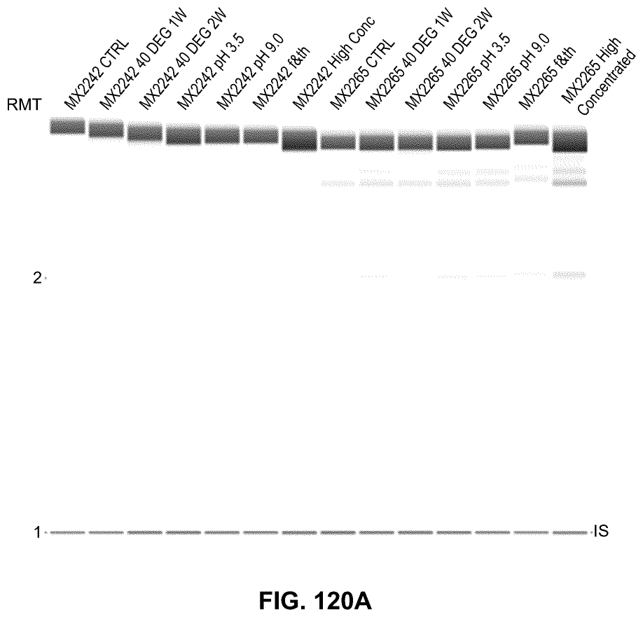

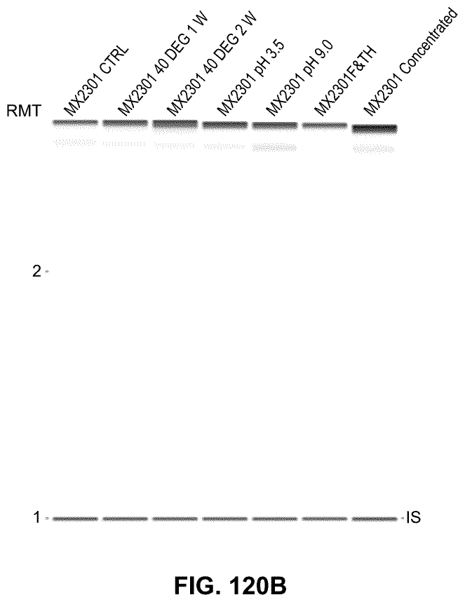

CE-SDS Analysis Under Stress Conditions: CE-SDS Analysis of MX2242, MX2265, and MX2301 was performed under different stress conditions: 40° C., for 1 week, 40° C., for 2 weeks, pH 3.5, pH 9.0, freeze and thaw, and high concentration. Half antibody contaminants were detected in all control and stressed samples of MX2242 and MX2265 (FIG. 120A). Migration of MX2301 was irregular, likely due to the presence of glycans (FIG. 120B).

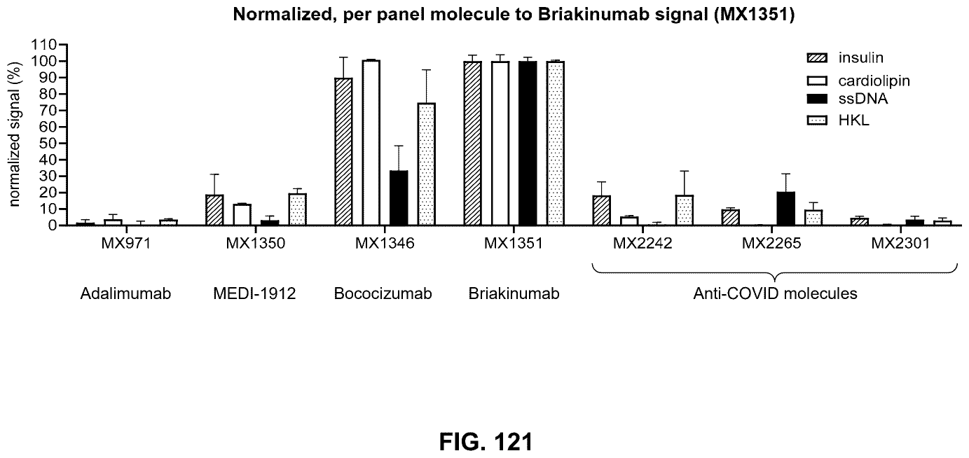

Poly-reactivity Analysis (ELISA panel): Poly-reactivity by ELISA panel was analyzed for MX2242, MX2265, and MX2301, and compared to control molecules. The tested anti-COVID molecules demonstrated low non-specific poly-reactivity, similar to MX2148 (FIG. 121. Table E74).

TABLE E74
Poly-reactivity by ELISA panel of MX2242, MX2265, and MX2301.

| | | Poly-reactivity score |
|---|---|---|
| | | (out of 100) |
| Adalimumab | MX971 | 2 |
| MEDI-1912 | MX1350 | 14 |
| Bococizumab | MX1346 | 75 |
| Briakinumab | MX1351 | 100 |
| Anti-COVID | MX2242 | 11 |
| molecules | MX2265 | 10 |
| | MX2301 | 3 |

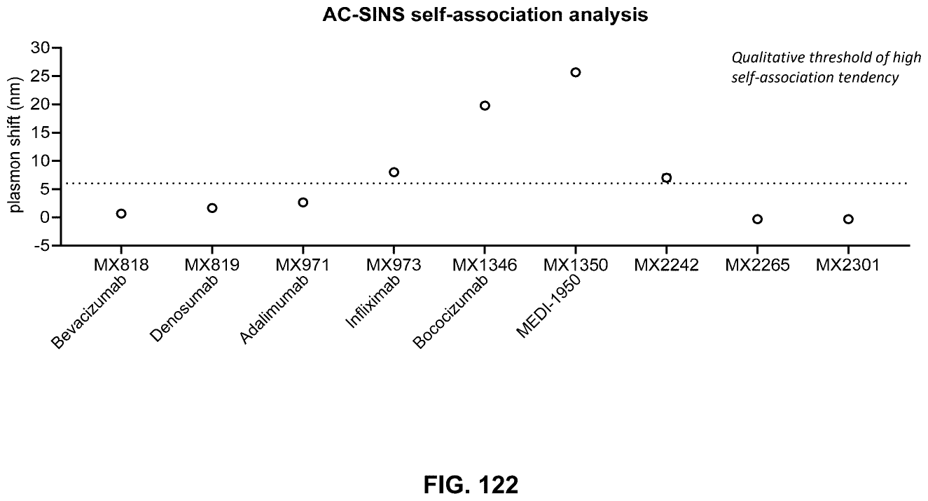

Self-interaction Assessment: Self-interaction was assessed for MX2242, MX2265, and MX2301 by AC-SINS (Affinity-Capture Self-Interacting Nanoparticle Spectroscopy), AC-SINS was performed in histidine with salt. Gold particles were coated with antibodies as indicated in FIG. 122, MX2265 and MX2301 did not display self-association tendency (FIG. 122). The de-glycosylation mutant MX2242 displayed moderate association tendency (FIG. 122).

Mass Analysis: Mass analysis of MX2242, MX2265, and MX2301 was performed. A C-terminal lysine residue was not observed in any of three tested molecules: MX2242, MX2265, and MX2301, although a lysine residue was included in their respective expression constructs. Upon PNGase F treatment, the intact mass of MX2242 matched to its theoretical mass (16 ppm). As for MX2265, both subunits of Knob_chain and Hole_chain matched to their calculated masses (less than 20 ppm). The B2 WT clone within MX2301 carried fully matured complex type N-glycans with two of them having terminal sialic acid residue (Neu5Ac), as desired.

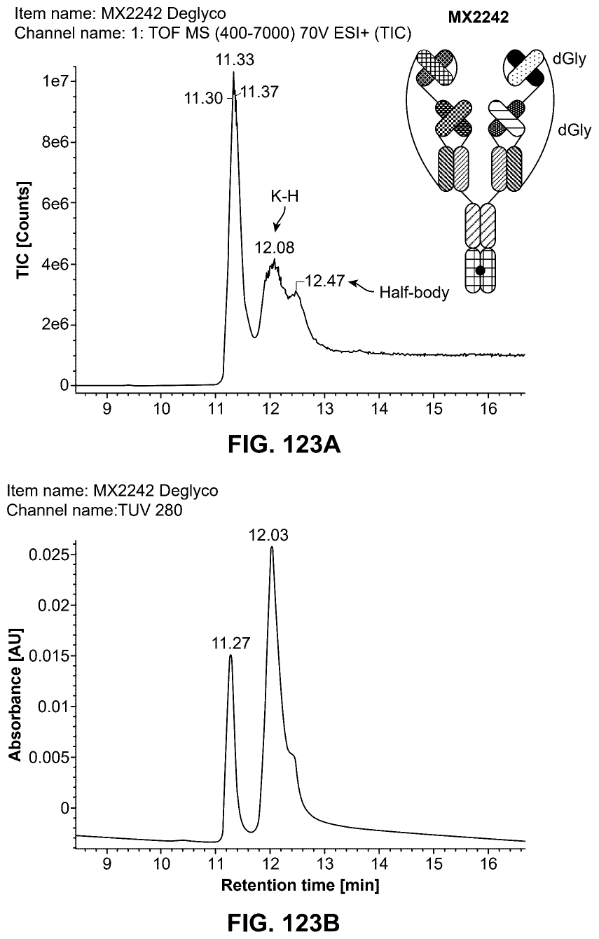

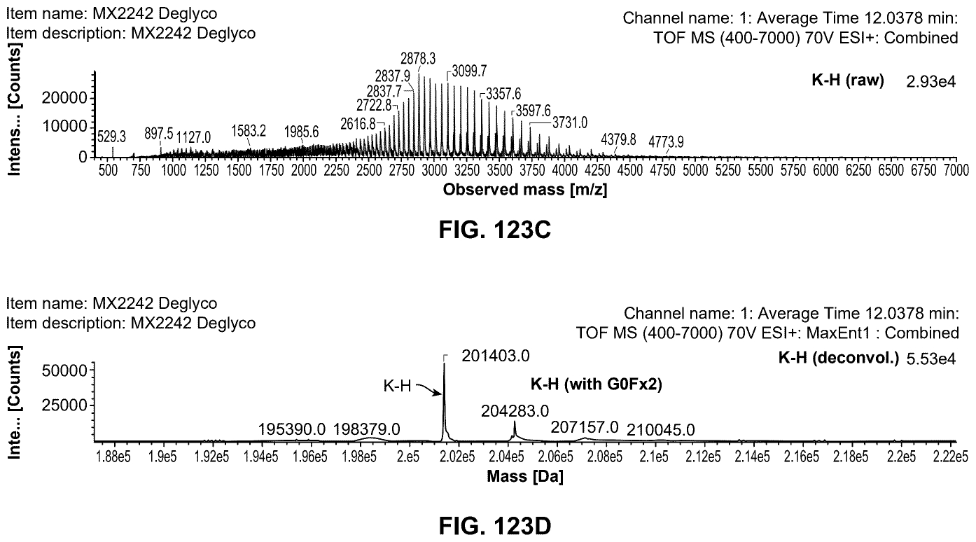

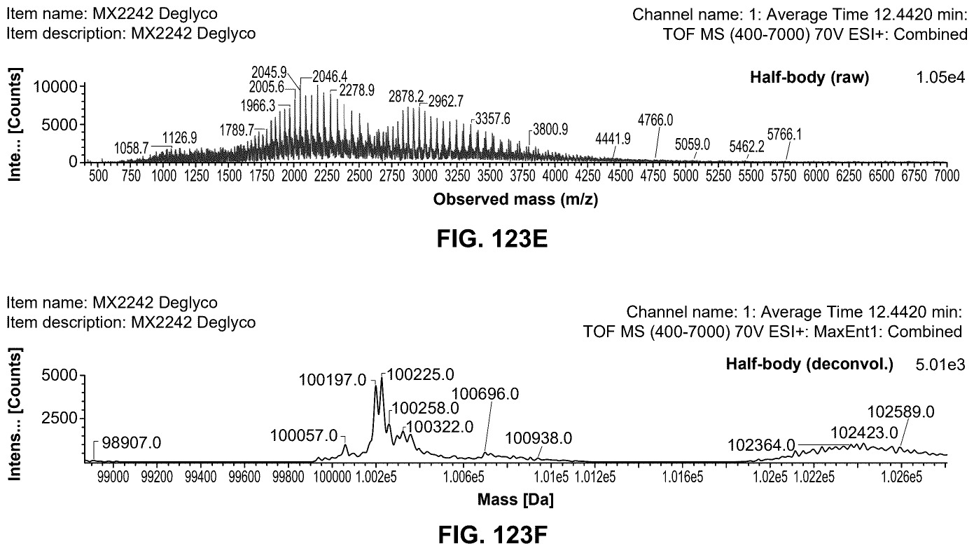

MX2242 (FIGS. 123A-123F. Table E75): By LC/MS analysis of MX2242 the desired Knob-Hole dimer was observed without a c-terminal Lys. Samples were treated with PNGase F. A major peak showed an intact mass matching to the correct MX2242 dimer (K-H). The intact mass didn't indicate the presence of a c-terminal Lysine residue. Fc N-glycans were not completely removed, mass at 204283 Da corresponded to K-H with G0Fx2. Some level of half-bodies were observed.

TABLE E75
LC/MS Analysis of MX2242.

| | | Calc. Ave. | Deglyco | Obs. | Diff. |
|---|---|---|---|---|---|
| MX2242 | | Mass (Da) | Mass | Mass | (ppm) |
| Without c-Terminal | K | 99934.77 | 99935.75 | | |
| Lys | H | 101465.14 | 101466.12 | | |
| | K-H | 201397.89 | 201399.85 | 201403 | 16 |
| | K-K | 199867.52 | 199869.48 | | |
| | H-H | 202928.26 | 202930.22 | | |
| With c-Terminal Lys | K | 100062.94 | 100063.92 | | |
| | H | 101593.31 | 101594.29 | | |
| | K-H | 201654.24 | 201656.2 | | |
| | K-K | 200123.87 | 200125.83 | | |
| | H-H | 203184.61 | 203186.57 | | |

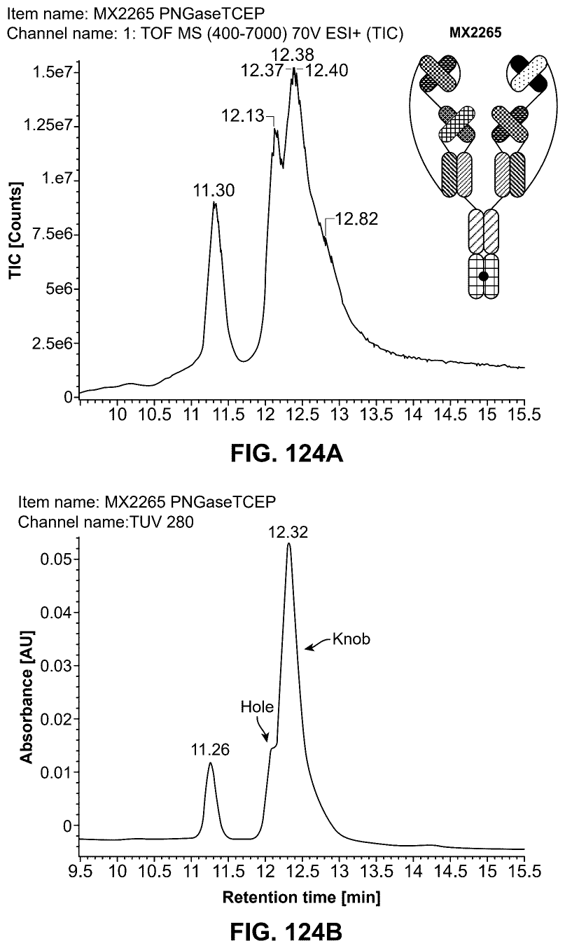

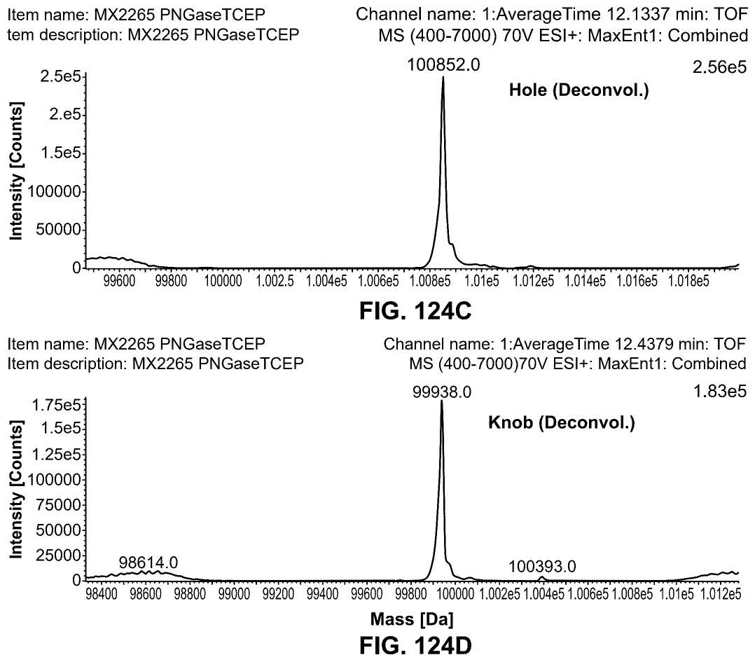

MX2265 (FIGS. 124A-124D. Table E76): LC/MS analysis of MX2265 showed that both Knob and Hole chains were matched. Samples were treated with PNGase F, and then with TCEP to yield Knob and Hole chains separately. Major peaks showed intact mass matching to the correct MX2265 Knob and Hole chains without any c-terminal lysine.

TABLE E76
LC/MS Analysis of MX2265.

| | | Calc. Ave. | Deglyco | Obs. | Diff. |
|---|---|---|---|---|---|
| MX2265 | | Mass (Da) | Mass | Mass | (ppm) |
| Without c-Terminal | K | 99938.81 | 99939.79 | 99938.0 | −18 |
| Lys | H | 100851.77 | 100852.75 | 100852.0 | −7 |
| | K-H | 200780.48 | 200782.44 | | |
| | K-K | 199867.52 | 199869.48 | | |
| | H-H | 201693.44 | 201695.4 | | |

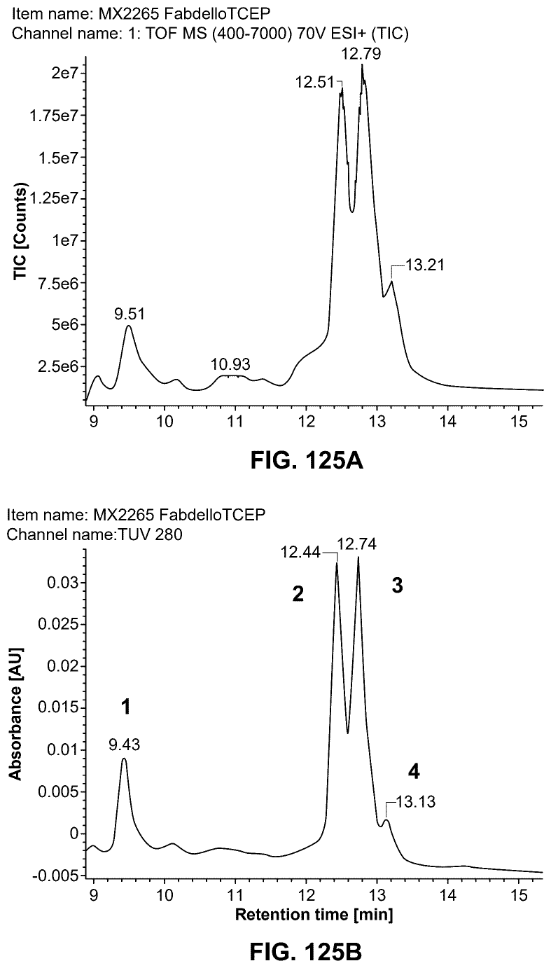

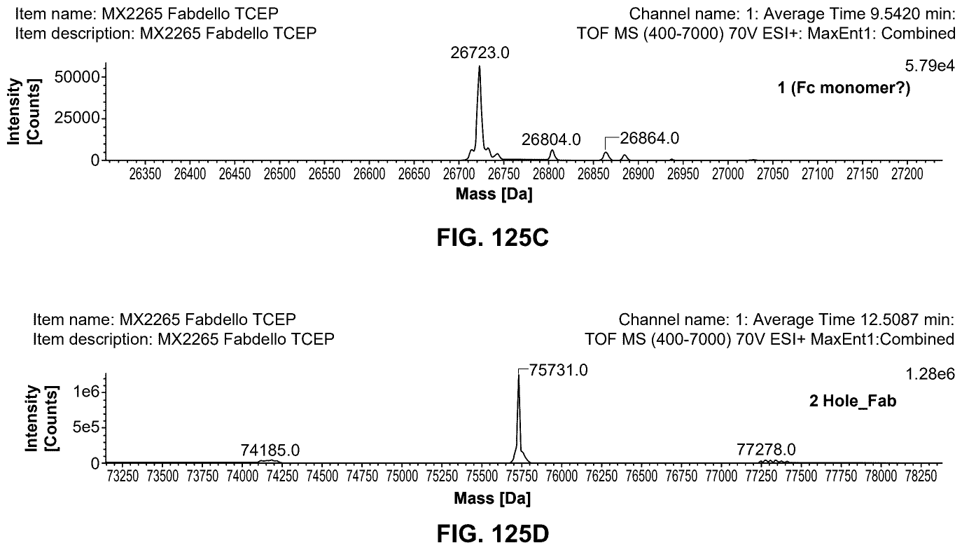

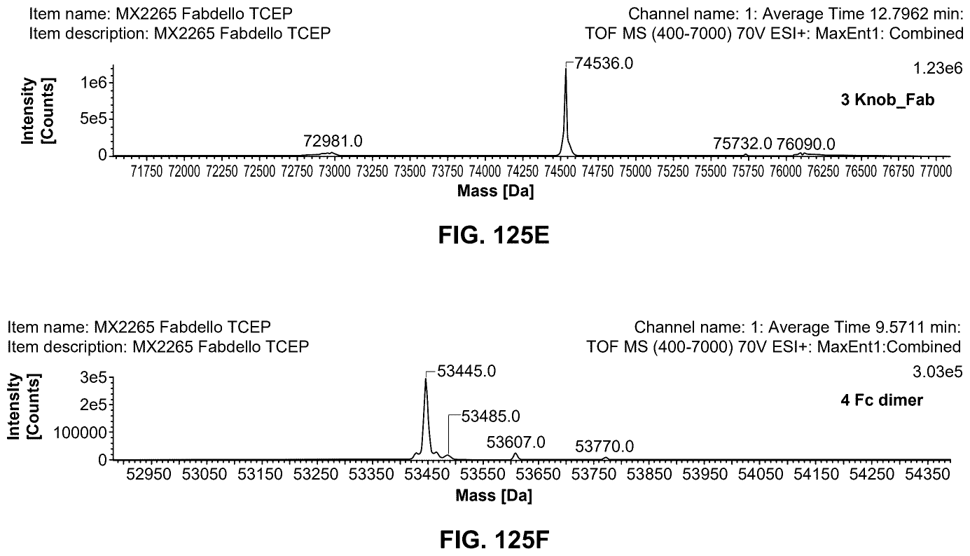

MX2265 (FIGS. 125A-125F. Table E77): LC/MS analysis of MX2265 showed that both Knob_Fab and Hole_Fab were matched, and that the C-terminal Lysine seemed to cause an unknown modification. Samples were treated with Fabdello enzyme, then with TCEP to yield Knob_Fab, hole_Fab. Fc_Knob, and Fc_Hole fragments, separately. Peaks #2 and #3, shown in FIG. 125B, corresponded to Hole_Fab and Knob_Fab, respectively. The small peak #4, shown in FIG. 125B, matched to un-reduced (pair) Fc_knob_hole_dimer without c-terminal lysine. Peak #1, shown in FIG. 125B, was related to Fc fragment, but didn't match to Knob-Fc and Hole_Fc. These data suggested not to include the c-terminal lysine, because it appeared to cause unknown modification(s), clipping, etc.

TABLE E77
LC/MS Analysis of MX2265.

| | | Calc. Ave. | Reduced | | | |
|---|---|---|---|---|---|---|
| MX2265 | | Mass (Da) | Mass | (GOF) | Obs. Mass | Diff. (ppm) |
| Without c- | Knob_Fab | 74535.29 | 74537.31 | | 74536.0 | −18 |
| Terminal Lys | Hole_Fab | 75729.6 | 75731.62 | | 75731 | −8 |
| | Knob_Fc | 25417.5 | 25420.53 | 26865.86 | 26723 | NOT Match |
| | Hole_Fc | 25136.14 | 25139.17 | 26584.5 | 26723 | NOT Match |
| | Fc_dimer | 50551.62 | 50551.62 | 53442.28 | 53445 | 51 |

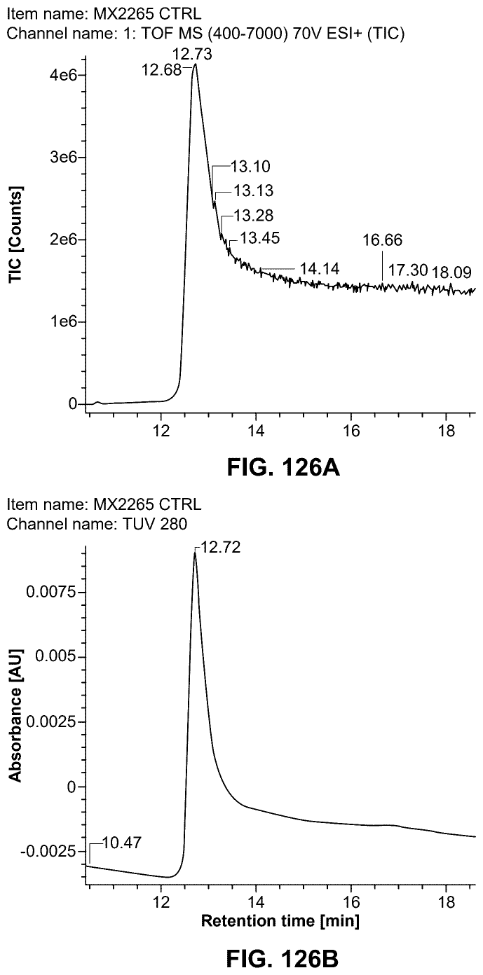

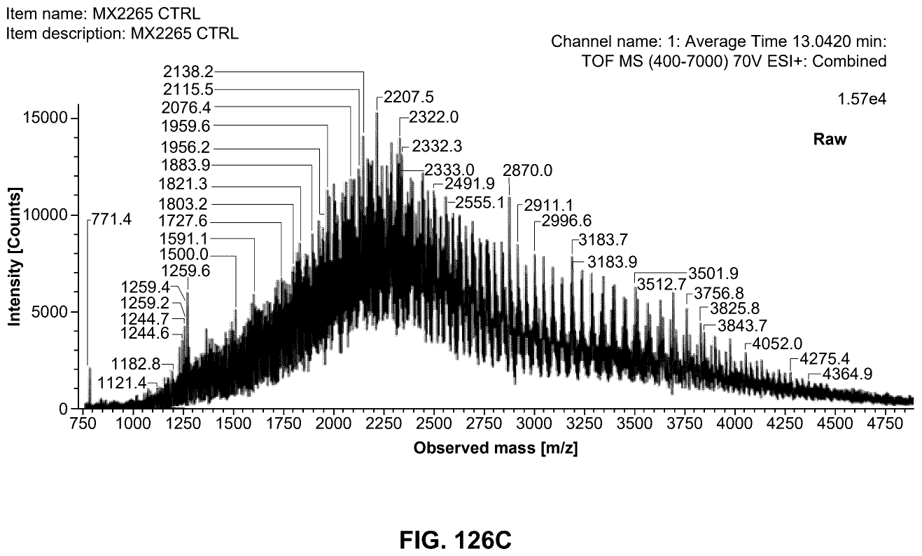

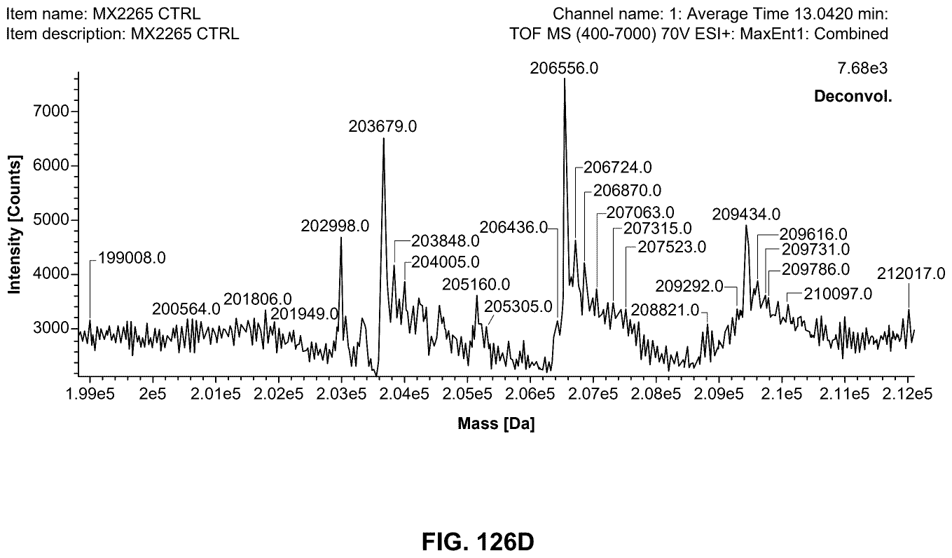

MX2265 (FIGS. 126A-126D. Table E78): By LC/MS analysis of non-treated MX2265 intact mass of Knob_Hole dimer was observed. In the absence of treatment for MX2265, a raw spectrum showed raised noise background due to Fc N-glycosylations. Upon one of deconvolution, masses were matching to the calculated Knob-Hole dimer. Some other masses were also observed. Improvements were observed without c-terminal lysine residue.

TABLE E78
LC/MS Analysis of MX2265.

| | | Calc. Ave. | | Obs. | Diff. |
|---|---|---|---|---|---|
| MX2265 | | Mass (Da) | G0F + G0F | Mass | (ppm) |
| Without c-Terminal | K | 99938.81 | | | |
| Lys | H | 100851.77 | | | |
| | K-H | 200780.48 | 203671.146 | 203679 | 39 |
| | K-K | 199867.52 | 202758.186 | | |
| | H-H | 201693.44 | 204584.106 | | |

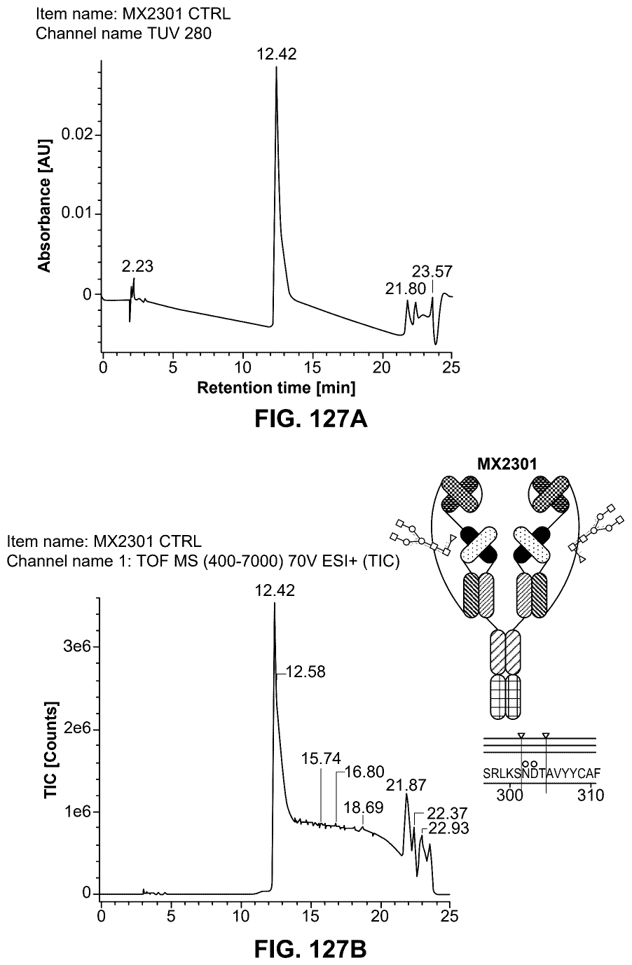

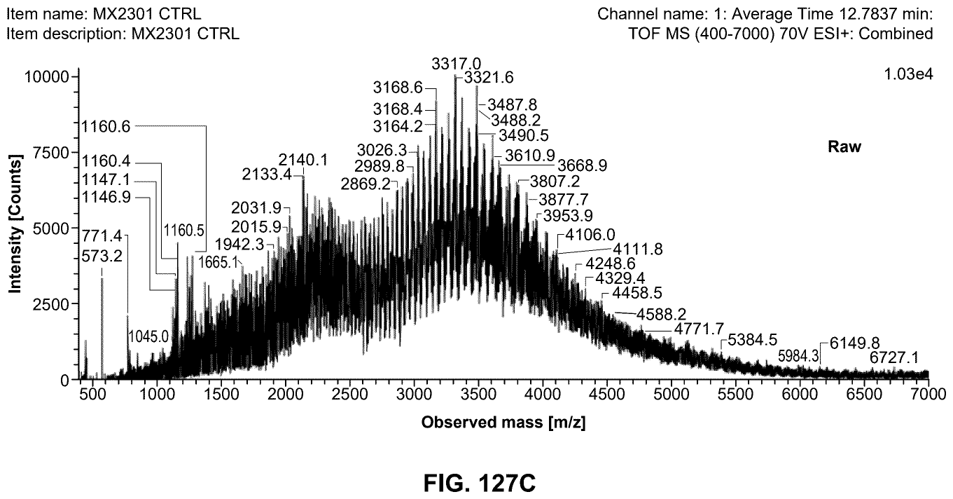

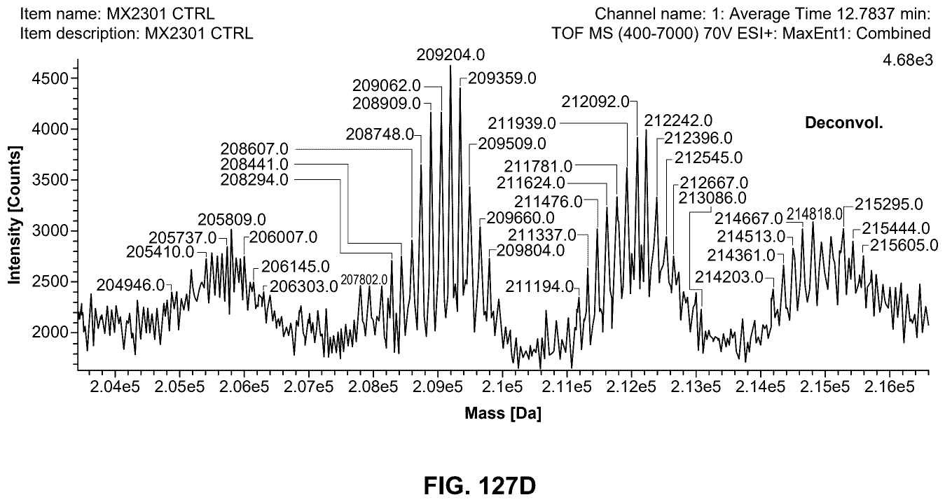

MX2301 (FIGS. 127A-127D): LC/MS Analysis of non-treated MX2301 showed heterogeneous mass profiles due to four N-glycosylation sites. In the absence of treatment for MX2301, raw spectrum showed raised noise background due to N-glycosylation (Fab “NDT” of B2 clone and Fc N-297). Upon deconvolution, a series of masses was observed due to glycosylation pattern (160 Da, 204 Da differences).

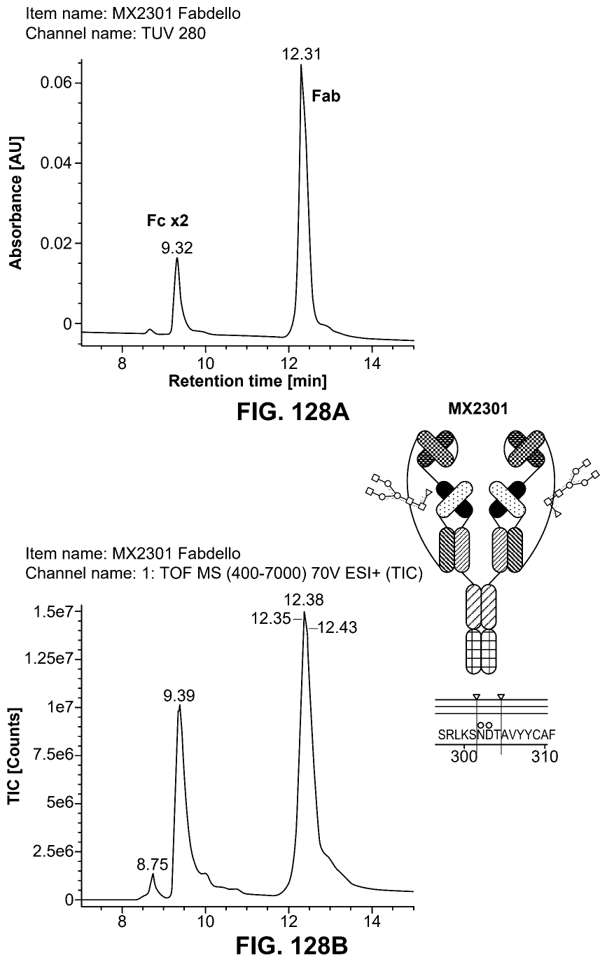

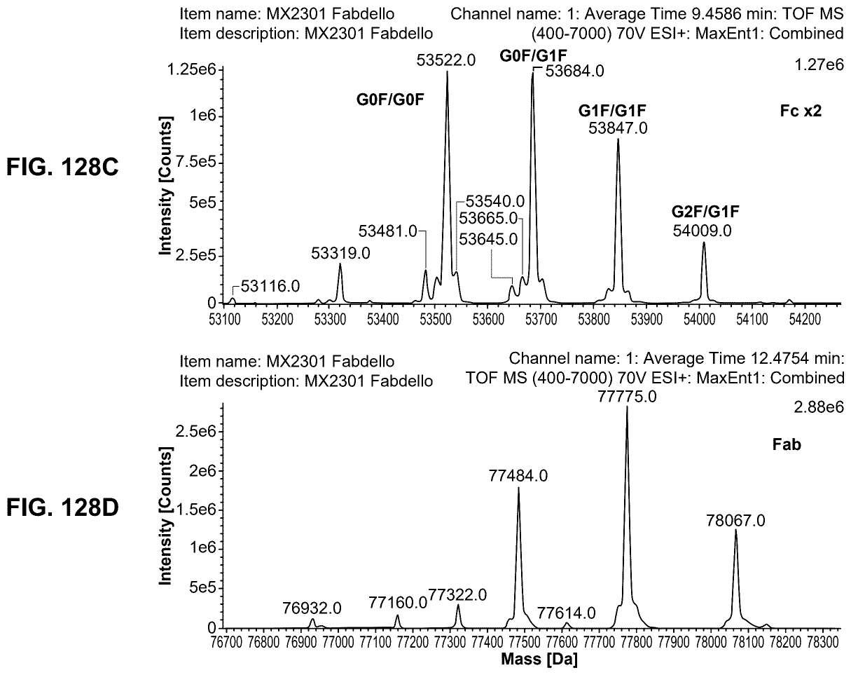

MX2301 (FIGS. 128A-128D, Table E79): Upon LC/MS analysis of Fc Subunit of MX2301 no c-terminal lysine was observed. Upon treatment with Fabdello, the subunit of Fcx2 dimer were matching to the masses without c-terminal lysine residues. The N-glycans observed are common to Fc N-glycosylation, e.g. G0F/G0F, G1F/G0F, G1F/G1F, etc.

TABLE E79
LC/MS Analysis of MX2301 (Without c-terminal Lys).

| | Calc. Ave. | Obs. | Diff. |
|---|---|---|---|
| MX2301 | Mass (Da) | Mass | (ppm) |
| Fc_dimer | 50632.65 | | |
| Fc_dimer + G0F/ G0F | 53523.31 | 53522 | −24 |
| Fc_dimer + G0F/GIF | 53685.45 | 53684 | −27 |
| Fc_dimer + G1F/GIF | 53847.59 | 53847 | −11 |
| Fc_dimer + G2F/GIF | 54009.73 | 54009 | −14 |

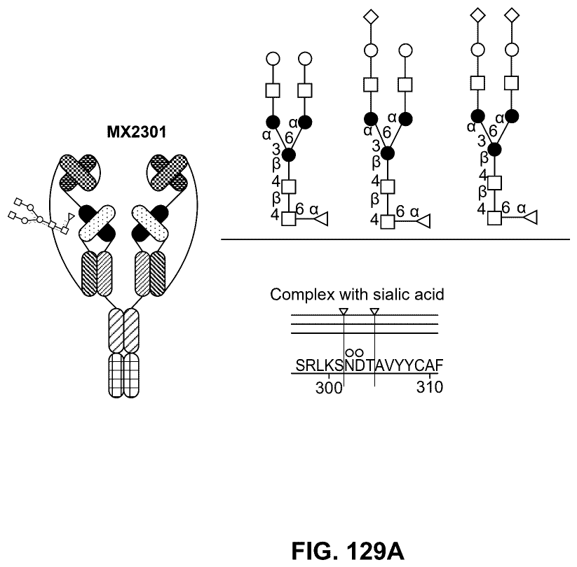

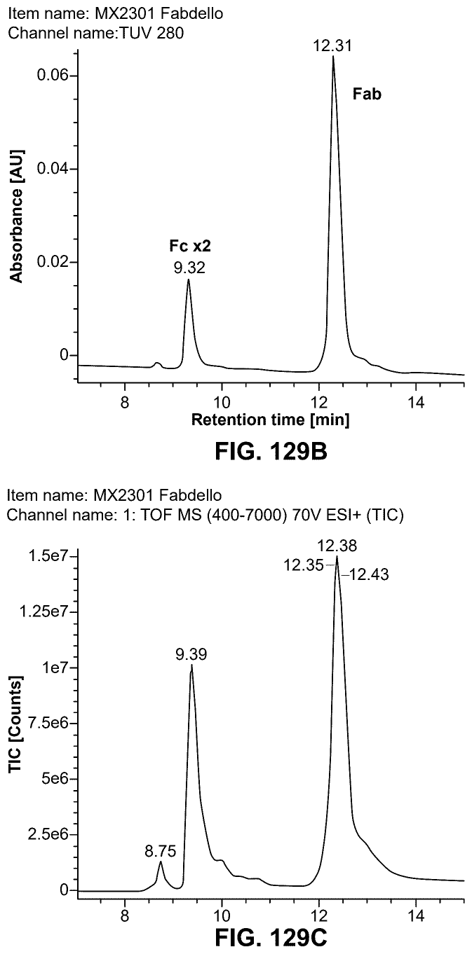

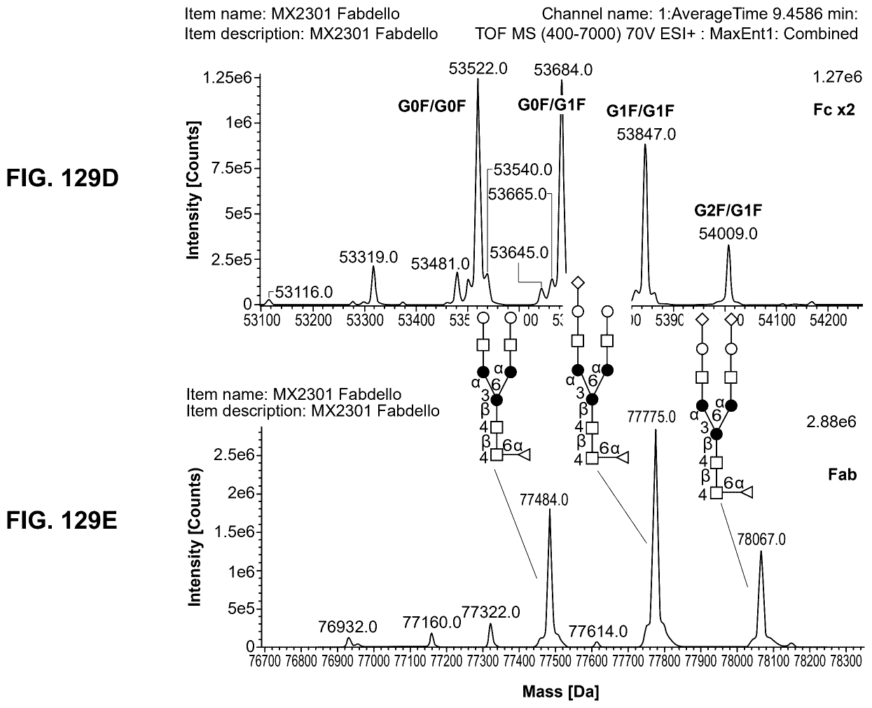

MX2301 (FIGS. 129A-129E, Table E80): LC/MS analysis of Fab Subunit of MX2301 showed that the B2 WT clone carries complex-type N-glycans with abundant sialic acid residues. Three most abundant N-glycans that were observed within Fab subunit (B2 clone) were complex type with two of them having terminal sialic acid residues (Neu5Ac), showing no concern from glycosylation perspective.

TABLE E80
LC/MS Analysis of MX2301.

| | Calc. | Obs. | Diff. |
|---|---|---|---|
| MX2301 | Mass (Da) | Mass | (ppm) |
| Fab | 75715.58 | | |
| Fab-Hex5HexNAc4Fuc1 | 77485.19 | 77484 | −15 |
| Fab-Hex5HexNAc4SA1Fuc1 | 77776.45 | 77775 | −19 |
| Fab-Hex5HexNAc4SA2Fuc1 | 78067.7 | 78067 | −9 |

Additional pH stress recovery data are shown in Tables E81 and E82.

TABLE E81
pH (3.5) stress recovery of MX2242, MX2265, and MX2301.

| pH 3.5 Stressed Sample | Recovery (%) |
|---|---|
| MX2242 | 99 |
| MX2265 | 100 |
| MX2301 | 100 |

TABLE E82
pH (9.0) stress recovery of MX2242, MX2265, and MX2301.

| pH 9.0 Stressed Sample | Recovery (%) |
|---|---|
| MX2242 | 92 |
| MX2265 | 100 |
| MX2301 | 100 |

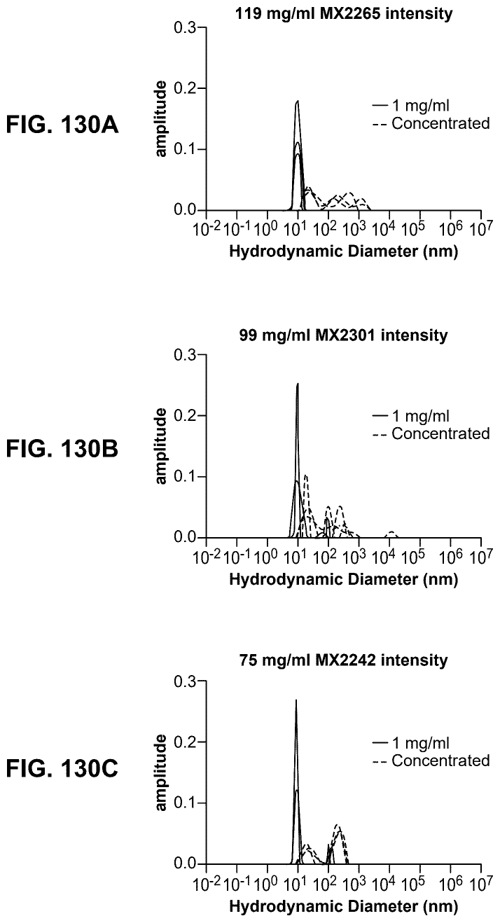

Additional data (DLS (intensity) about solubility at high concentration of MX2242, MX2265, and MX2301, are shown in FIGS. 130A-130C and in Table E83. Samples were formulated in 20 mM histidine, PH 6.0, 8% sucrose. All samples tested formed significant higher molecular weight assemblies at high concentration.

TABLE E83
Solubility at high concentration of MX2242, MX2265,
and MX2301 (DLS (intensity)).

| | Z-Ave. | | Pk 1 Peak | Pk 1 |
|---|---|---|---|---|
| Sample | Dia. (nm) | PDI | Dia. (nm) | Mass (%) |
| 1 mg/ml ref | 8.28 ± 0.14 | 0.28 ± 0.00 | 9.54 ± 0.00 | 99.99 ± 0.01 |
| MX2242 | | | | |
| 75 mg/ml | 67.70 ± 0.70 | 0.36 ± 0.00 | 21.71 ± 2.34 | 99.47 ± 0.16 |
| MX2242 | | | | |
| 1 mg/ml ref | 8.70 ± 0.65 | 0.29 ± 0.01 | 10.34 ± 0.00 | 100.00 ± 0.00 |
| MX2265 | | | | |
| 119 mg/ml | 75.34 ± 6.23 | 0.28 ± 0.02 | 24.40 ± 1.89 | 99.96 ± 0.25 |
| MX2265 | | | | |
| 1 mg/ml ref | 8.96 ± 0.17 | 0.29 ± 0.00 | 9.81 ± 0.38 | 99.97 ± 0.01 |
| MX2301 | | | | |
| 99 mg/ml | 41.71 ± 5.16 | 0.26 ± 0.03 | 20.84 ± 2.75 | 99.55 ± 0.37 |
| MX2301 | | | | |

Table E84 shows a summary of the developability analysis of MX2242, MX2265, and MX2301.

TABLE E84
Developability analysis of MX2242, MX2265, and MX2301
MX2242, MX2265, and MX2301 (Risk Level: Low ✓).

| | Biochemical and biophysical | | | |
|---|---|---|---|---|
| | properties | MX2242 | MX2265 | MX2301 |
| 1 | Overall purity assessment | ✓ | Below 95% by | ✓ |
| | | | aSEC ✓ | |
| 2 | Binding characterization* | ✓ | ✓ | ✓ |
| 4 | Surface hydrophobicity | ✓ | ✓ | Heterogeneous aHIC profile |
| | | | | ✓ |
| 5 | Surface charge (pI profile) | ✓ | pI 7.52-7.67 | pI 7.1-7.85 |
| 6 | Solubility at high concentration | Increasing hydrodynamic diameter | | |
| | (target to 100 mg/ml) | | | |
| 7 | Thermal stability (Tm, Tonset) | ✓ | ✓ | ✓ |
| 8 | Thermal Stress at 40° C. | Increasing HMW % | | |
| 9 | Freeze Thaw 5X | ✓ | ✓ | Increasing hydrodynamic |
| | | | | diameter ✓ |
| 10 | pH 3.5 Stress (RT for 4 hrs)* | increasing the peak tailing by aSEC | | |
| 11 | pH 9.0 Stress (RT for 24 hrs)* | increasing the peak tailing by aSEC | | |
| 12 | Serum/PBS 37° C. Stability* | Stable in | Stable in | Stable in serum/PBS-no |
| | | serum/PBS- | serum/PBS-no | change to aSEC or BLI |
| | | no change to | change to aSEC | binding |
| | | aSEC or BLI | or BLI binding | |
| | | binding | | |
| 13 | Poly-reactivity | ✓ | ✓ | ✓ |
| 14 | Self-association | ✓ | ✓ | ✓ |
| | LC/MS | ✓ | ✓ | Complex glycan on B2 FV ✓ |

LENGTHY TABLES
The patent application contains a lengthy table section. A copy of the table is available in electronic form from the USPTO web site (https://seqdata.uspto.gov/docdetail?docId=US20250326823A1). An electronic copy of the table will also be available from the USPTO upon request and payment of the fee set forth in 37 CFR 1.19(b)(3).

1-572. (canceled)

573. An antigen binding polypeptide complex comprising a first polypeptide and a second polypeptide,
wherein the first polypeptide has a structure, from amino-terminus to carboxy-terminus, represented by:
VL1-L1-CL-L2-VL2-L3-VH2-L4-VH1-L5-CH1-CH2-CH3;
VL1-L1-CL-L2-VH2-L3-VL2-L4-VH1-L5-CH1-CH2-CH3;
VL1-L1-CH1-L2-VL2-L3-VH2-L4-VH1-L5-CL-CH2-CH3;
VL1-L1-CH1-L2-VH2-L3-VL2-L4-VH1-L5-CL-CH2-CH3;
VH1-L1-CL-L2-VL2-L3-VH2-L4-VL1-L5-CH1-CH2-CH3;
VH1-L1-CL-L2-VH2-L3-VL2-L4-VL1-L5-CH1-CH2-CH3;
VH1-L1-CH1-L2-VL2-L3-VH2-L4-VL1-L5-CL-CH2-CH3;
VH1-L1-CH1-L2-VH2-L3-VL2-L4-VL1-L5-CL-CH2-CH3;
VL2-L1-CL-L2-VL1-L3-VH1-L4-VH2-L5-CH1-CH2-CH3;
VL2-L1-CL-L2-VH1-L3-VL1-L4-VH2-L5-CH1-CH2-CH3;
VL2-L1-CH1-L2-VL1-L3-VH1-L4-VH2-L5-CL-CH2-CH3;
VL2-L1-CH1-L2-VH1-L3-VL1-L4-VH2-L5-CL-CH2-CH3;
VH2-L1-CL-L2-VL1-L3-VH1-L4-VL2-L5-CH1-CH2-CH3;
VH2-L1-CL-L2-VH1-L3-VL1-L4-VL2-L5-CH1-CH2-CH3;
VH2-L1-CH1-L2-VL1-L3-VH1-L4-VL2-L5-CL-CH2-CH3; or
VH2-L1-CH1-L2-VH1-L3-VL1-L4-VL2-L5-CL-CH2-CH3;
wherein the second polypeptide has a structure, from amino-terminus to carboxy-terminus, represented by:
VL3-L6-CL-L7-VL4-L8-VH4-L9-VH3-L10-CH1-CH2-CH3;
VL3-L6-CL-L7-VH4-L8-VL4-L9-VH3-L10-CH1-CH2-CH3;
VL3-L6-CH1-L7-VL4-L8-VH4-L9-VH3-L10-CL-CH2-CH3;
VL3-L6-CH1-L7-VH4-L8-VL4-L9-VH3-L10-CL-CH2-CH3;
VH3-L6-CL-L7-VL4-L8-VH4-L9-VL3-L10-CH1-CH2-CH3;
VH3-L6-CL-L7-VH4-L8-VL4-L9-VL3-L10-CH1-CH2-CH3;
VH3-L6-CH1-L7-VL4-L8-VH4-L9-VL3-L10-CL-CH2-CH3;
VH3-L6-CH1-L7-VH4-L8-VL4-L9-VL3-L10-CL-CH2-CH3;
VL4-L6-CL-L7-VL3-L8-VH3-L9-VH4-L10-CH1-CH2-CH3;
VL4-L6-CL-L7-VH3-L8-VL3-L9-VH4-L10-CH1-CH2-CH3;
VL4-L6-CH1-L7-VL3-L8-VH3-L9-VH4-L10-CL-CH2-CH3;
VL4-L6-CH1-L7-VH3-L8-VL3-L9-VH4-L10-CL-CH2-CH3;
VH4-L6-CL-L7-VL3-L8-VH3-L9-VL4-L10-CH1-CH2-CH3;
VH4-L6-CL-L7-VH3-L8-VL3-L9-VL4-L10-CH1-CH2-CH3;
VH4-L6-CH1-L7-VL3-L8-VH3-L9-VL4-L10-CL-CH2-CH3; or
VH4-L6-CH1-L7-VH3-L8-VL3-L9-VL4-L10-CL-CH2-CH3; and
wherein:
VL1 is a first immunoglobulin light chain variable region that specifically binds to a severe acute respiratory syndrome coronavirus 2 (SARS-CoV-2) protein;
VL2 is a second immunoglobulin light chain variable region that specifically binds to a SARS-CoV-2 protein;
VL3 is a third immunoglobulin light chain variable region that specifically binds to a SARS-CoV-2 protein;
VL4 is a fourth immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
VH4 is a fourth immunoglobulin heavy chain variable region that specifically binds to a SARS-CoV-2 protein;
CL is an immunoglobulin light chain constant region;
CH1 is an immunoglobulin heavy chain constant region 1;
CH2 is an immunoglobulin heavy chain constant region 2;
CH3 is an immunoglobulin heavy chain constant region 3;
L1-L10 are optional amino acid linkers; and
wherein the CH2 and the CH3 are comprised in an Fc region and wherein the Fc region further comprises an immunoglobulin hinge region.

574. The antigen binding polypeptide complex of claim 573, wherein:
VL1 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766;
VL2 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764;
VL3 comprises a CDR1 having an amino acid sequence at least 65%, at least 70%, at least 75%, at least 80%, at least 85%, at least 90%, at least 95%, at least 99%, or 100% identical to SEQ ID NO: 152 or 1045, a CDR2 having an amino acid sequence at least 65%, at least 70%, at least 75%, at least 80%, at least 85%, at least 90%, at least 95%, at least 99%, or 100% identical to SEQ ID NO: 1046 or an amino acid sequence at least 65%, at least 70%, at least 75%, at least 80%, at least 85%, at least 90%, at least 95%, at least 99%, or 100% identical to amino acid sequence GAS, and a CDR3 having an amino acid sequence at least 65%, at least 70%, at least 75%, at least 80%, at least 85%, at least 90%, at least 95%, at least 99%, or 100% identical to SEQ ID NO: 153 or 766;
VL4 comprises a CDR1 having an amino acid sequence at least 65%, at least 70%, at least 75%, at least 80%, at least 85%, at least 90%, at least 95%, at least 99%, or 100% identical to SEQ ID NO: 145 or 1050, a CDR2 having an amino acid sequence at least 65%, at least 70%, at least 75%, at least 80%, at least 85%, at least 90%, at least 95%, at least 99%, or 100% identical to SEQ ID NO: 1051 or an amino acid sequence at least 65%, at least 70%, at least 75%, at least 80%, at least 85%, at least 90%, at least 95%, at least 99%, or 100% identical to amino acid sequence AAS, and a CDR3 having an amino acid sequence at least 65%, at least 70%, at least 75%, at least 80%, at least 85%, at least 90%, at least 95%, at least 99%, or 100% identical to SEQ ID NO: 146 or 764;
VH1 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049;
VH2 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054;
VH3 comprises a CDR1 having an amino acid sequence at least 65%, at least 70%, at least 75%, at least 80%, at least 85%, at least 90%, at least 95%, at least 99%, or 100% identical to SEQ ID NO: 155 or 1047, a CDR2 having an amino acid sequence at least 65%, at least 70%, at least 75%, at least 80%, at least 85%, at least 90%, at least 95%, at least 99%, or 100% identical to SEQ ID NO: 156 or 1048, and a CDR3 having an amino acid sequence at least 65%, at least 70%, at least 75%, at least 80%, at least 85%, at least 90%, at least 95%, at least 99%, or 100% identical to SEQ ID NO: 157, 767, or 1049; and
VH4 comprises a CDR1 having an amino acid sequence at least 65%, at least 70%, at least 75%, at least 80%, at least 85%, at least 90%, at least 95%, at least 99%, or 100% identical to SEQ ID NO: 148 or 1052, a CDR2 having an amino acid sequence at least 65%, at least 70%, at least 75%, at least 80%, at least 85%, at least 90%, at least 95%, at least 99%, or 100% identical to SEQ ID NO: 149 or 1053, and a CDR3 having an amino acid sequence at least 65%, at least 70%, at least 75%, at least 80%, at least 85%, at least 90%, at least 95%, at least 99%, or 100% identical to SEQ ID NO: 150, 765, or 1054.

575. The antigen binding polypeptide complex of claim 574, wherein:
VL1 comprises an amino acid sequence that is at least 65%, at least 70%, at least 75%, at least 80%, at least 85%, at least 90%, at least 95%, at least 99%, or 100% identical to SEQ ID NO: 154;
VL2 comprises an amino acid sequence that is at least 65%, at least 70%, at least 75%, at least 80%, at least 85%, at least 90%, at least 95%, at least 99%, or 100% identical to SEQ ID NO: 147;
VL3 comprises an amino acid sequence that is at least 65%, at least 70%, at least 75%, at least 80%, at least 85%, at least 90%, at least 95%, at least 99%, or 100% identical to SEQ ID NO: 154;
VL4 comprises an amino acid sequence that is at least 65%, at least 70%, at least 75%, at least 80%, at least 85%, at least 90%, at least 95%, at least 99%, or 100% identical to SEQ ID NO: 147;
VH1 comprises an amino acid sequence that is at least 65%, at least 70%, at least 75%, at least 80%, at least 85%, at least 90%, at least 95%, at least 99%, or 100% identical to SEQ ID NO: 158;
VH2 comprises an amino acid sequence that is at least 65%, at least 70%, at least 75%, at least 80%, at least 85%, at least 90%, at least 95%, at least 99%, or 100% identical to SEQ ID NO: 151;
VH3 comprises an amino acid sequence that is at least 65%, at least 70%, at least 75%, at least 80%, at least 85%, at least 90%, at least 95%, at least 99%, or 100% identical to SEQ ID NO: 158; and
VH4 comprises an amino acid sequence that is at least 65%, at least 70%, at least 75%, at least 80%, at least 85%, at least 90%, at least 95%, at least 99%, or 100% identical to SEQ ID NO: 151.

576. The antigen binding polypeptide complex of claim 575, wherein the first polypeptide comprises an amino acid sequence at least 65%, at least 70%, at least 75%, at least 80%, at least 85%, at least 90%, at least 95%, at least 99%, or 100% identical to SEQ ID NO: 989 and the second polypeptide comprises an amino acid sequence at least 65%, at least 70%, at least 75%, at least 80%, at least 85%, at least 90%, at least 95%, at least 99%, or 100% identical to SEQ ID NO: 990.

577. The antigen binding polypeptide complex of claim 573, wherein the antigen binding polypeptide complex is an antibody or antigen binding fragment thereof.

578. A chimeric antigen receptor (CAR) comprising the antigen binding polypeptide complex of claim 573.

579. An immune cell comprising the CAR of claim 578.

580. A polypeptide having at least 65%, at least 70%, at least 75%, at least 80%, at least 85%, at least 90%, at least 95%, at least 99%, or 100% identity to any one of SEQ ID NOs: 381-502, 632-633, 720-721, 724-725, 728-729, 732-733, 736-737, 740-741, 744-745, 748-749, 752-753, 760-761, 887-926, 972-977, 984-985, 989-990, 993-994, 997-998, 1001-1002, 1005-1006, 1009-1010, 1013-1014, 1017-1018, 1021-1022, 1025-1026, 1029-1030, 1033-1034, 1037-1038, and 1041-1042.

581. A polynucleotide having at least 65%, at least 70%, at least 75%, at least 80%, at least 85%, at least 90%, at least 95%, at least 99%, or 100% identity to any one of SEQ ID NOs: 503-625, 718-719, 722-723, 726-727, 730-731, 734-735, 738-739, 742-743, 746-747, 750-751, 754-755, 762-763, 927-966, 978-983, 986-987, 991-992, 995-996, 999-1000, 1003-1004, 1007-1008, 1011-1012, 1015-1016, 1019-1020, 1023-1024, 1027-1028, 1031-1032, 1035-1036, 1039-1040, and 1043-1044.

582. A vector comprising the polynucleotide of claim 581.

583. A host cell comprising the vector of claim 582.

584. An mRNA encoding the polypeptide of claim 580.

585. A lipid nanoparticle (LNP) comprising the mRNA of claim 584.

586. A pharmaceutical composition comprising the antigen binding polypeptide complex of claim 573 and a pharmaceutically acceptable carrier.

587. A method of preventing or treating a SARS-CoV-2 viral infection in a subject in need thereof, comprising administering to the subject a therapeutically effective amount of the antigen binding polypeptide complex of claim 573.

588. A method of preventing or treating coronavirus disease 2019 (COVID-19) in a subject in need thereof, comprising administering to the subject a therapeutically effective amount of the antigen binding polypeptide complex of claim 573.

589. A method of diagnosing a subject as being infected with a SARS-CoV-2 virus or suspected of being infected with a SARS-CoV-2 virus, comprising (i) contacting a sample obtained from the subject with the antigen binding polypeptide complex of claim 573; (ii) detecting the presence or absence of a virus complex which contains the antigen binding polypeptide complex or a fragment thereof and a SARS-CoV-2 virus, virion, or fragment thereof; and (iii) diagnosing the subject as being infected with a SARS-CoV-2 virus or suspected of being infected with a SARS-CoV-2 virus when the presence of the virus complex is detected.

590. A method of diagnosing a subject as not being infected with a SARS-CoV-2 virus or not suspected of being infected with a SARS-CoV-2 virus, comprising (i) contacting a sample obtained from the subject with antigen binding polypeptide complex of claim 573; (ii) detecting the presence or absence of a virus complex which contains the antigen binding polypeptide complex or a fragment thereof and a SARS-CoV-2 virus, virion, or fragment thereof; and (iii) diagnosing the subject as not being infected with a SARS-CoV-2 virus or not suspected of being infected with a SARS-CoV-2 virus when the presence of the virus complex is not detected.

591. A method of diagnosing a subject as having COVID-19 or suspected of having COVID-19, comprising (i) contacting a sample obtained from the subject with the antigen binding polypeptide complex of claim 573; (ii) detecting the presence or absence of a virus complex which contains the antigen binding polypeptide complex or a fragment thereof and a SARS-CoV-2 virus, virion, or fragment thereof; and (iii) diagnosing the subject as having COVID-19 or suspected of having COVID-19 when the presence of the virus complex is detected.

592. A method of diagnosing a subject as not having COVID-19 or not suspected of having COVID-19, comprising (i) contacting a sample obtained from the subject with the antigen binding polypeptide complex of claim 573; (ii) detecting the presence or absence of a virus complex which contains the antigen binding polypeptide complex or a fragment thereof and a SARS-CoV-2 virus, virion, or fragment thereof; and (iii) diagnosing the subject as not having COVID-19 or not suspected of having COVID-19 when the presence of the virus complex is not detected.

593. An antigen binding polypeptide complex comprising a first polypeptide and a second polypeptide,
wherein the first polypeptide has a structure, from amino-terminus to carboxy-terminus, represented by:
VL1-L1-CL-L2-VL2-L3-VH2-L4-VH1-L5-CH1-CH2-CH3;
VL1-L1-CL-L2-VH2-L3-VL2-L4-VH1-L5-CH1-CH2-CH3;
VL1-L1-CH1-L2-VL2-L3-VH2-L4-VH1-L5-CL-CH2-CH3;
VL1-L1-CH1-L2-VH2-L3-VL2-L4-VH1-L5-CL-CH2-CH3;
VH1-L1-CL-L2-VL2-L3-VH2-L4-VL1-L5-CH1-CH2-CH3;
VH1-L1-CL-L2-VH2-L3-VL2-L4-VL1-L5-CH1-CH2-CH3;
VH1-L1-CH1-L2-VL2-L3-VH2-L4-VL1-L5-CL-CH2-CH3;
VH1-L1-CH1-L2-VH2-L3-VL2-L4-VL1-L5-CL-CH2-CH3;
VL2-L1-CL-L2-VL1-L3-VH1-L4-VH2-L5-CH1-CH2-CH3;
VL2-L1-CL-L2-VH1-L3-VL1-L4-VH2-L5-CH1-CH2-CH3;
VL2-L1-CH1-L2-VL1-L3-VH1-L4-VH2-L5-CL-CH2-CH3;
VL2-L1-CH1-L2-VH1-L3-VL1-L4-VH2-L5-CL-CH2-CH3;
VH2-L1-CL-L2-VL1-L3-VH1-L4-VL2-L5-CH1-CH2-CH3;
VH2-L1-CL-L2-VH1-L3-VL1-L4-VL2-L5-CH1-CH2-CH3;
VH2-L1-CH1-L2-VL1-L3-VH1-L4-VL2-L5-CL-CH2-CH3; or
VH2-L1-CH1-L2-VH1-L3-VL1-L4-VL2-L5-CL-CH2-CH3;
wherein the second polypeptide has a structure, from amino-terminus to carboxy-terminus, represented by:
VL3-L6-CL-L7-VL4-L8-VH4-L9-VH3-L10-CH1-CH2-CH3;
VL3-L6-CL-L7-VH4-L8-VL4-L9-VH3-L10-CH1-CH2-CH3;
VL3-L6-CH1-L7-VL4-L8-VH4-L9-VH3-L10-CL-CH2-CH3;
VL3-L6-CH1-L7-VH4-L8-VL4-L9-VH3-L10-CL-CH2-CH3;
VH3-L6-CL-L7-VL4-L8-VH4-L9-VL3-L10-CH1-CH2-CH3;
VH3-L6-CL-L7-VH4-L8-VL4-L9-VL3-L10-CH1-CH2-CH3;
VH3-L6-CH1-L7-VL4-L8-VH4-L9-VL3-L10-CL-CH2-CH3;
VH3-L6-CH1-L7-VH4-L8-VL4-L9-VL3-L10-CL-CH2-CH3;
VL4-L6-CL-L7-VL3-L8-VH3-L9-VH4-L10-CH1-CH2-CH3;
VL4-L6-CL-L7-VH3-L8-VL3-L9-VH4-L10-CH1-CH2-CH3;
VL4-L6-CH1-L7-VL3-L8-VH3-L9-VH4-L10-CL-CH2-CH3;
VL4-L6-CH1-L7-VH3-L8-VL3-L9-VH4-L10-CL-CH2-CH3;
VH4-L6-CL-L7-VL3-L8-VH3-L9-VL4-L10-CH1-CH2-CH3;
VH4-L6-CL-L7-VH3-L8-VL3-L9-VL4-L10-CH1-CH2-CH3;
VH4-L6-CH1-L7-VL3-L8-VH3-L9-VL4-L10-CL-CH2-CH3; or
VH4-L6-CH1-L7-VH3-L8-VL3-L9-VL4-L10-CL-CH2-CH3; and
wherein:
VL1 is a first immunoglobulin light chain variable region that specifically binds to (i) a tumor-associated antigen (TAA), (ii) an infectious disease antigen that is not a sarbecovirus antigen, or (iii) an other-pathology antigen;
VL2 is a second immunoglobulin light chain variable region that specifically binds to (i) a tumor-associated antigen (TAA), (ii) an infectious disease antigen that is not a sarbecovirus antigen, or (iii) an other-pathology antigen;
VL3 is a third immunoglobulin light chain variable region that specifically binds to (i) a tumor-associated antigen (TAA), (ii) an infectious disease antigen that is not a sarbecovirus antigen, or (iii) an other-pathology antigen;
VL4 is a fourth immunoglobulin light chain variable region that specifically binds to (i) a tumor-associated antigen (TAA), (ii) an infectious disease antigen that is not a sarbecovirus antigen, or (iii) an other-pathology antigen;
VH1 is a first immunoglobulin heavy chain variable region that specifically binds to (i) a tumor-associated antigen (TAA), (ii) an infectious disease antigen that is not a sarbecovirus antigen, or (iii) an other-pathology antigen;
VH2 is a second immunoglobulin heavy chain variable region that specifically binds to (i) a tumor-associated antigen (TAA), (ii) an infectious disease antigen that is not a sarbecovirus antigen, or (iii) an other-pathology antigen;
VH3 is a third immunoglobulin heavy chain variable region that specifically binds to (i) a tumor-associated antigen (TAA), (ii) an infectious disease antigen that is not a sarbecovirus antigen, or (iii) an other-pathology antigen;
VH4 is a fourth immunoglobulin heavy chain variable region that specifically binds to (i) a tumor-associated antigen (TAA), (ii) an infectious disease antigen that is not a sarbecovirus antigen, or (iii) an other-pathology antigen;
CL is an immunoglobulin light chain constant region;
CH1 is an immunoglobulin heavy chain constant region 1;
CH2 is an immunoglobulin heavy chain constant region 2;
CH3 is an immunoglobulin heavy chain constant region 3;
L1-L10 are optional amino acid linkers; and
wherein the CH2 and the CH3 are comprised in an Fc region and wherein the Fc region further comprises an immunoglobulin hinge region.

594. A method of preventing or treating (i) a cancer or a tumor, (ii) an infectious disease that is not a sarbecovirus infectious disease, or (iii) a pathology other than a cancer or a tumor or an infectious disease that is not a sarbecovirus infectious disease, comprising administering to the subject a therapeutically effective amount of the antigen binding polypeptide complex of claim 593.

595. A method of diagnosing a subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptide complex of claim 593; (b) detecting the presence or absence of a complex which contains the antigen binding polypeptide complex or a fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as having or as being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is detected.

596. A method of diagnosing a subject as not having or as not being suspected of having (i) a cancer or a tumor, (ii) an infectious disease that is not a sarbecovirus infectious disease, or (iii) a pathology other than a cancer or a tumor or an infectious disease that is not a sarbecovirus infectious disease, comprising (a) contacting a sample obtained from the subject with the antigen binding polypeptide complex of claim 593; (b) detecting the presence or absence of a complex which contains the antigen binding polypeptide complex or a fragment thereof and (i) a tumor-associated antigen (TAA) or fragment thereof, (ii) an infectious disease antigen that is not a sarbecovirus antigen or fragment thereof, or (iii) an other-pathology antigen or fragment thereof; and (c) diagnosing the subject as not having or as not being suspected of having (i) a cancer or a tumor, (ii) an infectious disease that is not a sarbecovirus infectious disease, or (iii) a pathology other than a cancer or a tumor or an infectious disease that is not a sarbecovirus infectious disease when the presence of the complex is not detected.
